(19) United States
(12) Patent Application Publication
(10) Pub. No.: US 2024/0368135 A1
(43) Pub. Date: Nov. 7, 2024

(54) SUBSTITUTED TETRAHYDROFURAN-2-CARBOXAMIDES AS MODULATORS OF SODIUM CHANNELS

(71) Applicant: VERTEX PHARMACEUTICALS INCORPORATED, Boston, MA (US)

(72) Inventors: Elizabeth Mary BECK, Abingdon, Oxfordshire (GB); Robert PULLIN, Oxford, Oxfordshire (GB); Gorka ETXEBARRIA JARDI, Badalona (ES); Dean STAMOS, Lexington, MA (US); Yvonne SCHMIDT, San Diego, CA (US); Joseph PONTILLO, San Diego, CA (US); Stephen Andrew THOMSON, Durham, NC (US); David Matthew SHAW, Oxford, Oxfordshire (GB); Nadia M. AHMAD, Hayes, Middlesex (GB); Lidio Marx CARVALHO MEIRELES, San Marcos, CA (US); Sarah SKERRATT, Cambridge, Cambridgeshire (GB); Sara S. HADIDA RUAH, La Jolla, CA (US); Mark Thomas Miller, Rancho Santa Fe, CA (US); Timothy Donald NEUBERT, San Diego, CA (US); Dennis James HURLEY, San Marcos, CA (US); Jinglan ZHOU, San Diego, CA (US); Alexander KINTZER, Carlsbad, CA (US); Steven John DURRANT, Headington, Oxford (GB); Christopher WRAY, Berkshire (GB); Anisa Nizarali VIRANI, Thatcham, Berkshire (GB); Kiri NORTH, Oxford, Oxfordshire (GB); James DODD, Wallingford, Oxfordshire (GB); Reece JACQUES, Oxford, Oxfordshire (GB); Stephen Michael GEDDIS, Oxford, Oxfordshire (GB); Bhairavi GALAN, Abingdon, Oxfordshire (GB); Ronald Marcellus KNEGTEL, Abingdon, Oxfordshire (GB); Ewa Iwona CHUDYK, Wantage, Oxfordshire (GB); Joanne Louise PINDER, Didcot, Oxfordshire (GB); Bruno Artur SOUSA, Reading, Berkshire (GB); Rebecca E. STEVENS, Gloucester, Gloucestershire (GB); Miranda Adele WRIGHT, Oxford, Oxfordshire (GB); Heather Clare TWIN, Wantage, Oxfordshire (GB); James Jun Bon MUI, Hertfordshire (GB)

(73) Assignee: VERTEX PHARMACEUTICALS INCORPORATED, Boston, MA (US)

(21) Appl. No.: 18/566,297

(22) PCT Filed: Jun. 3, 2022
(86) PCT No.: PCT/US2022/032238

§ 371 Date: Dec. 1, 2023

Related U.S. Application Data
(60) Provisional application No. 63/197,253, filed on Jun. 4, 2021.

(51) Int. Cl. C07D 405/12; A61K 31/341; A61K 31/351; A61K 31/38; A61K 31/397; A61K 31/4025; A61K 31/407; A61K 31/4155; A61K 31/416; A61K 31/4178; A61K 31/422; A61K 31/4245; A61K 31/427; A61K 31/437; A61K 31/443; A61K 31/4433; A61K 31/4439; A61K 31/4525; A61K 31/4545; A61K 31/4709; A61K 31/496; A61K 31/4995; A61K 31/501; A61K 31/506; A61K 31/5377; A61K 31/5383; A61K 31/55; A61K 31/551; A61K 31/665; C07D 307/24; C07D 405/14; C07D 407/12; C07D 409/12; C07D 413/12; C07D 413/14; C07D 417/12; C07D 471/04; C07D 487/04; C07D 487/08; C07D 491/048; C07D 491/107; C07D 498/04; C07D 498/08; C07F 9/655

(52) U.S. Cl. CPC C07D 405/12; A61K 31/341; A61K 31/351; A61K 31/38; A61K 31/397; A61K 31/4025; A61K 31/407; A61K 31/4155; A61K 31/416; A61K 31/4178; A61K 31/422; A61K 31/4245; A61K 31/427; A61K 31/437; A61K 31/443; A61K 31/4433; A61K 31/4439; A61K 31/4525; A61K 31/4545; A61K 31/4709; A61K 31/496; A61K 31/4995; A61K 31/501; A61K 31/506; A61K 31/5377; A61K 31/5383; A61K 31/55; A61K 31/551; A61K 31/665; C07D 307/24; C07D 405/14; C07D 407/12; C07D 409/12; C07D 413/12; C07D 413/14; C07D 417/12; C07D 471/04; C07D 487/04; C07D 487/08; C07D 491/048; C07D 491/107; C07D 498/04; C07D 498/08; C07F 9/65515

(57) ABSTRACT

Compounds of formula I and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

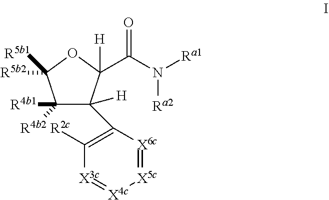

### CROSS-REFERENCE TO RELATED APPLICATIONS

This application claims the benefit of U.S. Provisional Application No. 63/197,253, filed Jun. 4, 2021, which is incorporated by reference herein in its entirety.

### BACKGROUND

Pain is a protective mechanism that allows healthy animals to avoid tissue damage and to prevent further damage to injured tissue. Nonetheless there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain. Clin. Ther., 2018 40(6): p. 828-49.

Voltage-gated sodium channels (Na_{V}s) are involved in pain signaling. Na_{V}s mediate the rapid upstroke of the action potential of many excitable cell types (e.g. neurons, skeletal myocytes, cardiac myocytes), and thus are involved in the initiation of electrical signaling in those cells (Hille, Bertil, Ion Channels of Excitable Membranes, Third ed. (Sinauer Associates, Inc., Sunderland, MA, 2001)). Support for the assertion that Na_{V}s play a critical and central role in pain signaling arises from (1) evaluation of the role Na_{V}s plays in normal physiology, (2) pathological states arising from mutations in the Na_{V}1.8 gene (SCN10A). (3) preclinical work in animal models, and (4) pharmacology of known Na_{V}1.8-modulating agents. In addition, because Na_{V}1.8 expression is restricted to peripheral neurons, particularly those that sense pain (e.g., the dorsal root ganglia), Na_{V}1.8 inhibitors are less likely to be associated with the side effects commonly observed with other sodium channel modulators and the abuse liability associated with opioid therapies. Therefore, targeting the underlying biology of pain through selective Na_{V}1.8 inhibition represents a novel approach to analgesic drug development that has the potential to address an urgent unmet need for safe and effective acute and chronic pain therapies (Rush, A. M. and T. R. Cummins, Painful Research: Identification of a Small-Molecule Inhibitor that Selectively Targets Na_{V}1.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Because of the role Na_{V}s play in the initiation and propagation of neuronal signals, antagonists that reduce Na_{V} currents can prevent or reduce neural signaling and Na_{V} channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Na_{V} channels. The local anesthetic drugs such as lidocaine block pain by inhibiting Na_{V} channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is the primary sodium channel of cardiac myocytes. Na_{V}s 1.7, 1.8, and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na+ current, TTX-resistant Na⁺ current, and Ca²⁺ current in the action potentials of nociceptive sensory neurons. J. Neurosci., 2002. 22(23): p. 10277-90). Na_{V}1.8 is involved in spontaneous firing in damaged neurons, like those that drive neuropathic pain (Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Na_{V}1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Na_{V}1.8. Pain, 2002. 95(1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286(46): p. 39836-47). The small DRG neurons where Na_{V}1.8 is expressed include the nociceptors involved in pain signaling. Na_{V}1.8 mediates large amplitude action potentials in small neurons of the dorsal root ganglia (Blair, N. T. and B. P. Bean, Roles of tetrodotoxin (TTX)-sensitive Na+ current, TTX-resistant Na+ current, and Ca²⁺ current in the action potentials of nociceptive sensory neurons. J. Neurosci., 2002. 22(23): p. 10277-90). Na_{V}1.8 is necessary for rapid repetitive action potentials in nociceptors and for spontaneous activity of damaged neurons. (Choi, J. S. and S. G. Waxman, Physiological interactions between Na_{V}1.7 and Na_{V}1.8 sodium channels: a computer simulation study. J. Neurophysiol. 106(6): p. 3173-84; Renganathan, M., T. R. Cummins, and S. G. Waxman, Contribution of Na(v)1.8 sodium channels to action potential electrogenesis in DRG neurons J. Neurophysiol., 2001. 86(2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6). In depolarized or damaged DRG neurons, Na_{V}1.8 appears to be a driver of hyper-excitablility (Rush, A. M., et al., A single sodium channel mutation produces hyper- or hypoexcitability in different types of neurons. Proc. Natl. Acad. Sci. USA, 2006. 103(21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J. Pain, 2008. 12(5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512(2): p. 61-6).

The inventors have discovered that some voltage-gated sodium channel inhibitors have limitations as therapeutic agents due to, for example, a poor therapeutic window (e.g., due to a lack of Na_{V} isoform selectivity, low potency, and/or other reasons). Accordingly, there remains a need to develop selective voltage-gated sodium channel inhibitors, such as selective Na_{V}1.8 inhibitors.

### SUMMARY

In one aspect, the invention relates to a compound described herein, or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### BRIEF DESCRIPTION OF THE DRAWINGS

FIG. 1 depicts an XRPD pattern characteristic of amorphous Compound 105.

FIG. 2 depicts an XRPD pattern characteristic of amorphous Compound 145.

FIG. 3 depicts an XRPD pattern characteristic of amorphous Compound 183.

FIG. 4 depicts an XRPD pattern characteristic of Compound 213 in partially crystalline form.

FIG. 5 depicts an XRPD pattern characteristic of amorphous Compound 215.

FIG. 6 depicts an XRPD pattern characteristic of amorphous Compound 263.

FIG. 7 depicts an XRPD pattern characteristic of amorphous Compound 334.

FIG. 8 depicts an XRPD pattern characteristic of amorphous Compound 360.

FIG. 9 depicts an XRPD pattern characteristic of amorphous Compound 525.

### DETAILED DESCRIPTION

In one aspect, the invention relates to a compound of formula (I)

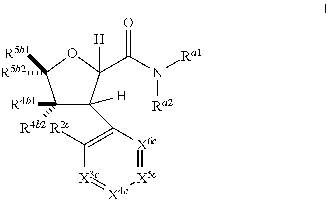

or a pharmaceutically acceptable salt thereof, wherein:
R^{a1} is —(C(R^{a′})₂)ₚ—R^{a″},

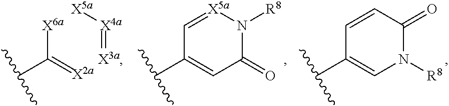

5-membered heteroaryl, 3-7 membered heterocycloalkyl, 9-10 membered aryl, or 9-10 membered heteroaryl, wherein said 5-membered heteroaryl, 3-7 membered heterocycloalkyl, 9-10 membered aryl, or 9-10 membered heteroaryl is optionally substituted by one or more R^{a3};
R^{a2} is H;
or R^{a1} and R^{a2} together with the nitrogen to which they are attached form a 3-10 membered heterocycloalkyl, wherein said 3-10 membered heterocycloalkyl is optionally substituted by one or more R^{a3};
each R^{a′} is independently H or methyl optionally substituted by —OH, or two R^{a′} together with the atom or atoms to which they are attached form C₃-C₆ cycloalkyl, 3-7 membered heterocycloalkyl, or oxo;
R^{a″} is C₃-C₆ cycloalkyl, 3-7 membered heterocycloalkyl, 5-10 membered heteroaryl, phenyl, —NR⁹R¹⁰, —OR¹¹, or —CN, wherein said 5-10 membered heteroaryl, 3-7 membered heterocycloalkyl, or phenyl is optionally substituted by one or more R¹³;
each R^{a3} is independently halo, C₁-C₆ alkyl, C₁-C₆ haloalkyl, 3-7 membered heterocycloalkyl, —C(O)C₁-C₆ alkyl, —OR¹¹, —C(O)NR⁹R¹⁰, or —S(O)₂R⁷, wherein said C₁-C₆ alkyl, C₁-C₆ haloalkyl, 3-7 membered heterocycloalkyl or —C(O)C₁-C₆ alkyl is optionally substituted by one or more halo, —OR¹¹, —CN, or —NR⁹R¹⁰, or two R^{a3} attached to the same atom combine to form oxo, or two R^{a3} attached to adjacent atoms together with the atoms to which they are attached combine to form a fused 3-7 membered ring containing up to two heteroatoms selected from the group consisting of N, O, and S;
X^{2a} is N, N⁺—O—, or C—R^{2a};
X^{3a} is N, N⁺—O—, or C—R^{3a};
X^{4a} is N, N⁺—O—, or C—R^{4a};
X^{5a} is N, N⁺—O—, C—R^{5a}, or N⁺—(C₁-C₆ alkyl)Y⁻, wherein Y⁻ is a monovalent anion;
X^{6a} is N, N⁺—O—, or C—R^{6a};
R^{2a} is H, halo, C₁-C₆ alkyl, or C₁-C₆ haloalkyl;
R^{3a} is H, halo, C₁-C₆ alkyl, C₁-C₆ haloalkyl, C₁-C₆ alkoxy, 3-9 membered heterocycloalkyl, 5-membered heteroaryl, —CN, —OR¹¹, —COOH, —NR⁹C(O)C₁-C₆ alkyl, —S(O)₂R⁷, —S(O)(NR⁹)R⁷, —S(O)NR⁹R¹⁰, —S(O)R⁷, or —P(O)(C₁-C₆ alkyl)₂, wherein said C₁-C₆ alkyl, C₁-C₆ alkoxy, 3-9 membered heterocycloalkyl, 5-membered heteroaryl, or —NR⁹C(O)C₁-C₆ alkyl is optionally substituted by one or more R¹², C₃-C₆ cycloalkyl, —NR⁹R¹⁰, —OR¹¹, —CN, or 3-7 membered heterocycloalkyl optionally substituted by one or more R²;
R^{4a} is H, halo, C₁-C₆ alkyl, C₁-C₆ haloalkyl, C₂-C₆ alkynyl, C₁-C₆ alkoxy, 3-7 membered heterocycloalkyl, 5-6 membered heteroaryl, —CN, —C(O)NR⁹R¹⁰, —C(O)OH, —OR¹¹, —NR⁹R¹⁰, —NR⁹C(O)C₁-C₆ alkyl, —S—C₁-C₆ alkyl, —S(O)(NR⁹)R⁷, —S(O)NR⁹R¹⁰, or —P(O)(C₁-C₆ alkyl)₂, wherein said C₁-C₆ alkyl, C₁-C₆ alkoxy, 3-7 membered heterocycloalkyl, 5-6 membered heteroaryl, or C₂-C₆ alkynyl is optionally substituted by one or more halo, —OR¹¹, 3-7 membered heterocycloalkyl, —NR⁹R¹⁰, C₁-C₆ alkyl, or —S(O)₂R⁷;
R^{5a} is H, halo, C₁-C₆ alkyl, C₁-C₆ haloalkyl, or —S(O)₂R⁷;
R^{6a} is H, halo, C₁-C₆ alkyl, or C₁-C₆ haloalkyl;
or R^{3a} and R^{4a} together with the atoms to which they are attached form a ring of formula:

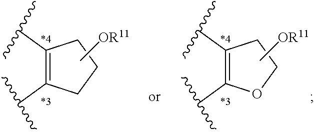

R⁷ is C₁-C₆ alkyl or 3-7 membered heterocycloalkyl, wherein said C₁-C₆ alkyl or 3-7 membered heterocycloalkyl is optionally substituted by one or more —OR¹¹ or C₁-C₆ alkyl;
R⁸ is H or C₁-C₆ alkyl;
R⁹ and R¹⁰ are each independently H, C₁-C₆ alkyl, 3-7 membered heterocycloalkyl, C₃-C₆ cycloalkyl, —OH, —CN, or —S(O)₂R⁷, wherein said C₁-C₆ alkyl is optionally substituted by one or more —OR¹¹, or R⁹ and R¹⁰ together with the atom to which they are attached form a 3-7 membered heterocycloalkyl;
each R¹¹ is independently H, C₁-C₆ alkyl, C₁-C₆ haloalkyl, a 3-7 membered heterocycloalkyl optionally substituted with —OH, or a 3-7 membered cycloalkyl optionally substituted with —OH; each R¹² is independently halo, C₁-C₆ alkyl, or —OR¹¹, or two R¹² together with the atom they are attached combine to form oxo;
each R¹³ is independently halo, C₁-C₆ alkyl, or —CONH₂, wherein said C₁-C₆ alkyl is optionally substituted by one or more —OR¹¹, or two R¹³ together with the atom they are attached combine to form oxo;
R^{4b1} and R^{4b2} are each independently H, C₁-C₆ alkyl, C₃-C₆ cycloalkyl, or C₁-C₆ haloalkyl; R^{5b1} and R^{5b2} are each independently H, C₁-C₆ alkyl, C₃-C₆ cycloalkyl, or C₁-C₆ haloalkyl;
X^{3c} is N or C—R^{3c};
X^{4c} is N or C—R^{4c};
X^{5c} is N or C—R^{5c};
X^{6c} is N or C—R^{6c};
R^{2c} is H, —OH, halo, C₁-C₆ alkyl, C₂-C₆ alkenyl, C₁-C₆ haloalkyl, C₁-C₆ alkoxy, C₁-C₆ haloalkoxy, -L¹-(C₁-C₆ alkylene)-OR¹⁵, -L¹-(C₁-C₆ alkenylene)-OR¹⁵, -L¹-(C₁-C₆ alkylene)-NR¹⁶R¹⁷, -L¹-(C₁-C₆ alkylene)-N═S(O)(C₁-C₃ alkyl)₂, or -L¹-L²-R¹⁴;
R¹⁴ is C₃-C₆ cycloalkyl, 3-8 membered heterocycloalkyl, 5- or 6-membered heteroaryl, —C(O)O(C₁-C₆ alkyl), —COOH, or —C(O)NR¹⁶R¹⁷, wherein said C₃-C₆ cycloalkyl, 3-8 membered heterocycloalkyl or 5- or 6-membered heteroaryl is optionally substituted by one or more halo, —OH, C₁-C₆ alkyl, C₁-C₆ haloalkyl, C₁-C₆ alkoxy, or C₁-C₆ haloalkoxy;
R¹⁵ is H, C₁-C₆ alkyl, or C₁-C₆ haloalkyl:
R¹⁶ and R¹⁷ are each independently H, —OH, C₁-C₆ alkyl, or 3-7 membered heterocycloalkyl;
R^{3c} is H, halo, C₁-C₆ alkyl, C₁-C₆ haloalkyl, or —(C₁-C₆ alkylene)-(C₁-C₆ alkoxy);
R^{4c} is H, halo, C₁-C₆ alkyl, or C₁-C₆ haloalkyl;
R^{5c} is H, halo, C₁-C₆ alkyl, or C₁-C₆ haloalkyl; and
R^{6c} is H, halo, C₁-C₆ alkyl, C₁-C₆ haloalkyl, or C₁-C₆ alkoxy;
L¹ is a bond or O;
L² is a bond or C₁-C₆ alkylene; and
p is 1, 2, or 3;
provided that no more than two of X^{2a}, X^{3a}, X^{4a}, X^{5a}, and X^{6a} are N or N⁺—O;
provided that no more than one of X^{3c}, X^{4c}, X^{5c}, and X^{6c} is N; and
provided that R^{4a} is not CH(OH)—R^{4a′}, wherein when R^{4a} is H or C₁-C₅ alkyl optionally substituted by one or more halo, —OR¹¹, 3-7 membered heterocycloalkyl, —NR⁹R¹⁰, C₁-C₆ alkyl, or —S(O)₂R⁷.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compounds of the invention” refers to the compounds of formula (I), and all of the embodiments thereof (e.g., formulas (I-A), etc.), as described herein, and to the compounds identified in Table A.

As described herein, the compounds of the invention comprise multiple variable groups (e.g., R^{aa}, X^{3a}, R^{5b1}, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and optionally their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The chemical structures depicted herein are intended to be understood as they would be understood by one of ordinary skill in the art. For example, with respect to the formula

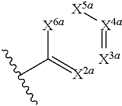

in the definition for R^{a1} associated with formula (I), X^{2a} and X^{3a} are connected by a single bond, and X^{5a} and X^{6a} are connected by a double bond, even though the bonds between these groups may be obscured by the atom labels in the chemical structures. Further, with respect to formulas (I), (I-A), (I-B), and (I-C), X^{4c} and X^{5c} are connected by a single bond, even though the bond between these groups may be obscured by the atom labels in the chemical structures. Moreover, a substituent depicted as “CF₃” or “F₃C” in a chemical structure refers to a trifluoromethyl substituent, regardless of which depiction appears in the chemical structure.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “alkenyl” refers to a straight or branched hydrocarbon chain radical group consisting solely of carbon and hydrogen atoms, containing one or more carbon-carbon double bonds, and having the specified number of carbon atoms, which is attached to the rest of the molecule by a single bond. For example, a “C₂-C₆ alkenyl” group is an alkenyl group having between two and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₅ cycloalkyl” group is a cycloalkyl group having between three and eight carbon atoms.

As used herein, the term “heterocycloalkyl” refers to a stable, non-aromatic, mono- or bicyclic (fused, bridged, or spiro) saturated hydrocarbon radical consisting carbon, hydrogen, and one or more hetero atoms such as nitrogen, oxygen, and sulfur, having the specified number of ring atoms, and which is attached to the rest of the molecule by a single bond. For example, a “3-7 membered heterocycloalkyl” group is a cycloalkyl group having between three and 7 atoms and having at least one heteroatom such as nitrogen, oxygen, and sulfur.

As used herein, the term “fused 3-7 membered ring containing up to two heteroatoms selected from the group consisting of N, O, and S,” when used in relation to a ring formed by two R³ groups attached to adjacent atoms together with the atoms to which they are attached, refers to a saturated, unsaturated, or aromatic ring fused to a heteroaryl, heterocycloalkyl, or aryl ring and containing up to two heteroatoms selected from the group consisting of N, O, and S.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “alkoxy” refers to a radical of the formula —ORₐ where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkoxy” refers to an alkoxy group having the specified number of carbon atoms, wherein one or more of the hydrogen atoms of the of the alkyl group are replaced by halo groups.

As used herein, the term “alkylene” refers to a divalent, straight or branched hydrocarbon chain radical group consisting solely of carbon and hydrogen atoms, containing no unsaturation, and having the specified number of carbon atoms, which is attached to the rest of the molecule by two single bonds. For example, a “C₁-C₆ alkylene” group is an alkylene group having between one and six carbon atoms.

As used herein the term “alkenylene” refers to a divalent, straight or branched hydrocarbon chain radical group consisting solely of carbon and hydrogen atoms, containing at least one carbon-carbon double bond, and having the specified number of carbon atoms, which is attached to the rest of the molecule by two single bonds. For example, a “C₁-C₆ alkenylene” group is an alkenylene group having between one and six carbon atoms.

As used herein the term “alkynyl” refers to a straight or branched hydrocarbon chain radical group consisting solely of carbon and hydrogen atoms, containing no unsaturation, and having the specified number of carbon atoms, which is attached to the rest of the molecule by a single bond and wherein the bond between any two of the other carbon atoms is a triple bond. For example, a “C₁-C₆ alkyl” group is an alkyl group having between one and six carbon atoms. For example, a “C₂-C₆ alkynyl” group is an alkynyl group having between 2 and 6 carbon atoms, wherein the bond between any two of the carbon atoms is a triple bond.

As used herein, the term “aryl” refers to a stable, aromatic, mono- or bicyclic ring radical having the specified number of ring atoms. For example, a “9-10 membered aryl” group is an aryl group having between nine and ten carbons.

As used herein, the term “heteroaryl” refers to a stable, aromatic, mono- or bicyclic ring radical having the specified number of ring atoms and comprising one or more heteroatoms individually selected from nitrogen, oxygen and sulfur.

As used herein, the term “monovalent anion” refers to an anion bearing a single unit of negative charge. In some embodiments, the monovalent anion is pharmaceutically acceptable. As used herein, the term “pharmaceutically acceptable monovalent anion” refers to those monovalent anions which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable monovalent anions include any of the monovalent anions that are components of the pharmaceutically acceptable salts described herein. Illustratively, the monovalent anion can be a halide, such as chloride or bromide, a hydroxide, a carboxylate, a sulfate, a phosphate, a nitrate, a lower alkyl sulfonate, and an aryl sulfonate. Illustrative carboxylates include halogenated carboxylates such as acetate and trifluoroacetate.

As used herein, the term “optionally substituted” refers to a group that is either unsubstituted or substituted with the subsequently identified substituents. For example, a group that is “optionally substituted with 1-2 halo” is either unsubstituted, substituted with 1 halo group, or substituted with 2 halo groups.

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, labels such as “*4” and “*3”, such as those shown in the following structure, designate the atoms to which the corresponding R groups (in this case, the R^{4a} and R^{3a} groups, respectively) are attached.

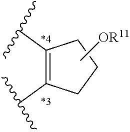

As used herein, in any chemical structure or formula, a non-bold, straight bond attached to a stereocenter of a compound, such as in

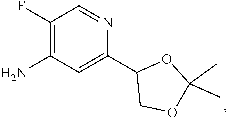

denotes that the configuration of the stereocenter is unspecified. The compound may have any configuration, or a mixture of configurations, at the stereocenter.

As used herein, in any chemical structure or formula, a bold or hashed straight bond attached to a stereocenter of a compound, such as in

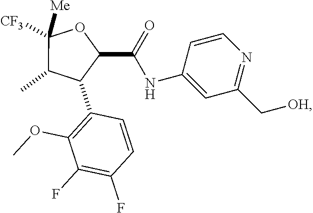 [SMILES: COc1c([C@H]2[C@H](C(=O)Nc3ccnc(CO)c3)O[C@@](C)(C(F)(F)F)[C@H]2C)ccc(F)c1F]

denotes the relative stereochemistry of the stereocenter, relative to other stereocenter(s) to which bold or hashed straight bonds are attached.

As used herein, in any chemical structure or formula, a bold or hashed wedge bond attached to a stereocenter of a compound, such as in

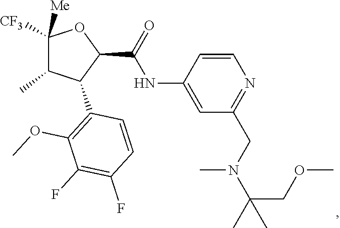

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. Where the relative stereochemistry of a given stereocenter is unknown, no stereochemical designator is provided. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

As used herein, the term “amorphous” refers to a solid material having no long-range order in the position of its molecules. Amorphous solids are generally glasses or supercooled liquids in which the molecules are arranged in a random manner so that there is no well-defined arrangement, e.g., molecular packing, and no long-range order. Amorphous solids are generally rather isotropic, i.e., exhibit similar properties in all directions and do not have definite melting points. Instead, they typically exhibit a glass transition temperature which marks a transition from glassy amorphous state to supercooled liquid amorphous state upon heating. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See US 2004/0006237 for a comparison of XRPDs of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. See US 2004/0006237 for a representative comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

As used herein, the term “crystalline” refers to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, and solid state nuclear magnetic resonance (e.g., ¹³C, ¹⁹F, ¹⁵N, and ³¹P SSNMR).

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position at at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (I-A)

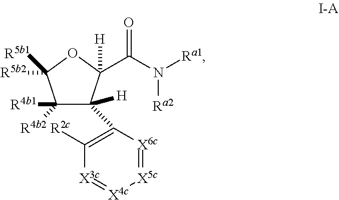

or a pharmaceutically acceptable salt thereof, wherein R^{a1}, R^{a2}, R^{4b1}, R^{4b2}, R^{5b1}, R^{5b2}, X^{3c}, X^{4c}, X^{5c}, X^{6c}, and R^{2c} are defined as set forth above in connection with formula (I).

In some embodiments, the invention relates to a compound of formula (I-A-1)

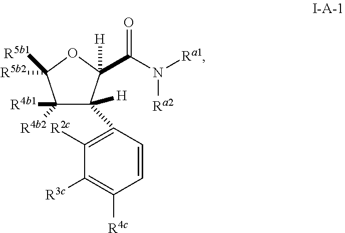

or a pharmaceutically acceptable salt thereof, wherein R^{a1}, R^{a2}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-A-2)

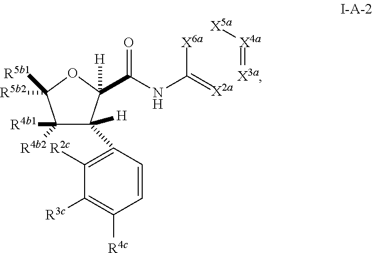

or a pharmaceutically acceptable salt thereof, wherein X^{2a}, X^{3a}, X^{4a}, X^{5a}, X^{6a}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-A-3)

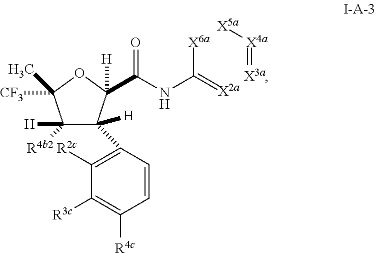

or a pharmaceutically acceptable salt thereof, wherein X^{2a}, X^{3a}, X^{4a}, X^{5a}, X^{6a}, R^{4b2}, R^{2c}, R^{3c}, and R^{4c} are defined as set forth above in connection with formula (I).

In some embodiments, the invention relates to a compound of formula (I-B)

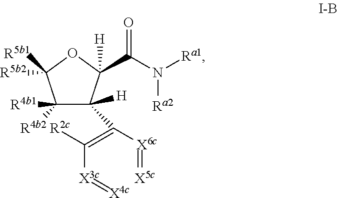

or a pharmaceutically acceptable salt thereof, wherein R^{a1}, R^{a2}, R^{4b1}, R^{4b2}, R^{5b1}, R^{5b2}, X^{3c}, X^{4c}, X^{5c}, X^{6c}, and R^{2c} are defined as set forth above in connection with formula (I).

In some embodiments, the invention relates to a compound of formula (I-B-1)

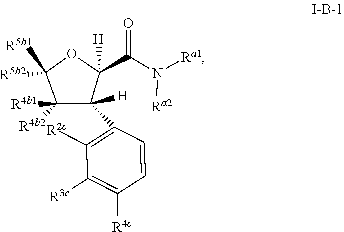

or a pharmaceutically acceptable salt thereof, wherein R^{a1}, R^{a2}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-B-2)

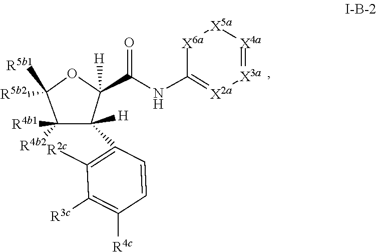

or a pharmaceutically acceptable salt thereof, wherein X^{2a}, X^{3a}, X^{4a}, X^{5a}, X^{6a}, R^{4b2}, R^{4b2}, R^{5b1}, R^{5b2}, R^{2c}, R^{3c}, and R^{4c} are defined as set forth above in connection with formula (I).

In some embodiments, the invention relates to a compound of formula (I-B-3)

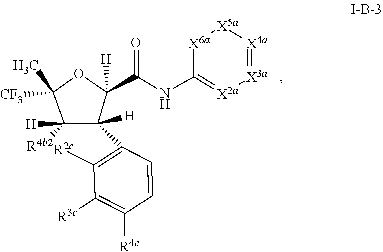

or a pharmaceutically acceptable salt thereof, wherein X^{2a}, X^{3a}, X^{4a}, X^{5a}, X^{6a}, R^{4b2}, R^{2c}, R^{3c}, and R^{4c} are defined as set forth above in connection with formula (I).

In some embodiments, the invention relates to a compound of formula (I-C)

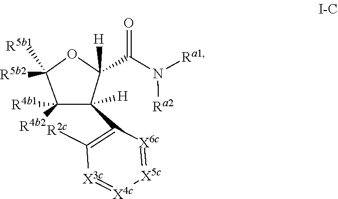

or a pharmaceutically acceptable salt thereof, wherein R^{a1}, R^{a2}, R^{4b1}, R^{4b2}, R^{5b1}, R^{5b2}, X^{3c}, X^{4c}, X^{5c}, X^{6c}, and R^{2c} are defined as set forth above in connection with formula (I).

In some embodiments, the invention relates to a compound of formula (I-C-1)

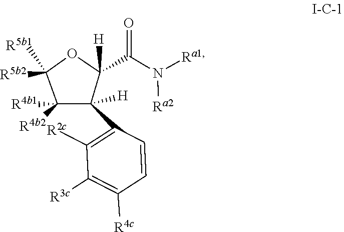

or a pharmaceutically acceptable salt thereof, wherein R^{a1}, R^{a2}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-C-2)

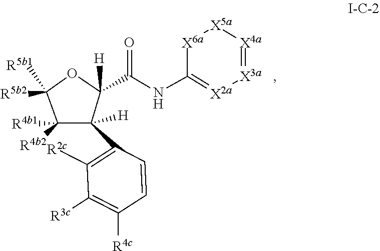

or a pharmaceutically acceptable salt thereof, wherein X^{2a}, X^{3a}, X^{4a}, X^{5a}, X^{6a}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-C-3)

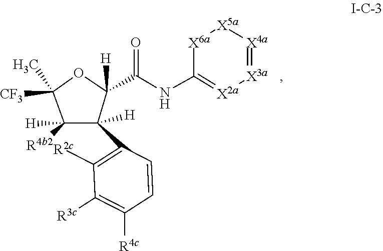

or a pharmaceutically acceptable salt thereof, wherein X^{2a}, X^{3a}, X^{4a}, X^{5a}, X^{6a}, R^{4b2}, R^{2c}, R^{3c}, and R^{4c} are defined as set forth above in connection with formula (I).

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein X^{2a} is C—R^{2a}. In other embodiments, X^{2a} is C—R^{2a}; and R^{2a} is H.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein X^{3a} is N. In other embodiments, X^{3a} is N⁺—O—. In other embodiments, X^{3a} is C—R^{3a}. In other embodiments, R^{3a} is —S(O)₂R⁷, —S(O)(NR⁹)R⁷, —S(O)NR⁹R¹⁰, —S(O)R⁷. In other embodiments, R^{3a} is —S(O)₂R⁷. In other embodiments, R^{3a} is —S(O)(NR⁹)R⁷. In other embodiments, R^{3a} is —S(O)NR⁹R¹⁰. In other embodiments, R^{3a} is —S(O)R⁷. In other embodiments, R⁷ is methyl. In other embodiments, R⁹ and R¹⁰ are methyl. In other embodiments, R^{3a} is C₁-C₆ alkyl, optionally substituted with —NR⁹R¹⁰, or —OR¹¹.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein X^{4a} is N.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein X^{5a} is N or C—R^{5a}; and R^{5a} is H, halo, or —CH₂OH. In other embodiments, X^{5a} is N. In other embodiments, X^{5a} is C—R^{5′}. In other embodiments, X^{5a} is C—R^{5a}; and R^{5a} is H, halo, or C₁-C₆ alkyl. In other embodiments, X^{5a} is C—R^{5a}, and R^{5a} is H, F, or C₁-C₆ alkyl. In other embodiments, X^{5a} is C—R^{5a}, and R^{5a} is H. In other embodiments, X^{5a} is C—R^{5a}, and R^{5a} is halo. In other embodiments, X^{5a} is C—R^{5a}, and R^{5a} is F. In other embodiments, X^{5a} is C—R^{5a}, and R^{5a} is CH₃.

In some embodiments, the invention relates to a compound of any one of formulas (I), (I-A), (I-B), and (I-C), or a pharmaceutically acceptable salt thereof, wherein X^{6a} is N or C—R^{6a}, and R^{6a} is H. In other embodiments, X^{6a} is N. In other embodiments, X^{6a} is C—R^{6a}. In other embodiments, X^{6a} is C—R^{6a} and R^{6a} is H.

In some embodiments, the invention relates to a compound of any one of formulas (I), (I-A), (I-A-1), (I-B), (I-B-1), (I-C), and (I-C-1), or a pharmaceutically acceptable salt thereof, wherein R^{a1} is

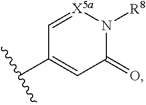

and R^{a2} is H. In other embodiments, R^{a1} is

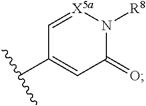

R^{a2} is H; X^{5a} is H; and R⁸ is H or CH₃. In other embodiments, R^{a1} is

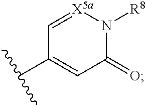

R^{a2} is H; X^{5a} is H; and R⁸ is CH₃.

In some embodiments, the invention relates to a compound of any one of (I), (I-A), (I-A-1), (I-B), (I-B-1), (I-C), and (I-C-1), or a pharmaceutically acceptable salt thereof, wherein R^{a1} is

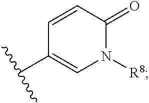

and R^{a2} is H. In other embodiments, R^{a1} is

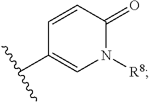

R^{a2} is H; and R⁸ is H or CH₃. In other embodiments, R^{a1} is

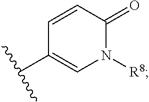

R^{a2} is H; and R⁸ is CH₃.

In some embodiments, the invention relates to a compound of any one of (I), (I-A), (I-A-1), (I-B), (I-B-1), (I-C), and (I-C-1), or a pharmaceutically acceptable salt thereof, wherein R^{a1} is 5-membered heteroaryl optionally substituted by one or more R^{a3}, and R^{a2} is H. In other embodiments, R^{a1} is 9-10 membered aryl optionally substituted by one or more R^{a3}, and R^{a2} is H. In other embodiments, R^{a1} is 9-10 membered heteroaryl optionally substituted by one or more R^{a3}, and R^{a2} is H. In other embodiments, R^{a3} is C₁-C₆ alkyl, C₁-C₆ haloalkyl, —OR¹¹, —C(O)NR⁹R¹⁰, or —S(O)₂R⁷. In other embodiments, R^{a3} is —S(O)₂—CH₃, —CH(OH)—CH(OH)—CH₃, —CH(OH)—CH₂—OH. In other embodiments, R^{a3} is —S(O)₂—CH₃,

In some embodiments, the invention relates to a compound of any one of formulas (I), (I-A), (I-A-1), (I-A-2), (I-B), (I-B-1), (I-B-2), (I-C), (I-C-1), and (I-C-2), or a pharmaceutically acceptable salt thereof, wherein R^{4b1} is H or C₁-C₆ alkyl. In other embodiments, R^{4b1} is H. In other embodiments, R^{4b1} is C₁-C₆ alkyl. In other embodiments, R^{4b1} is H or CH₃. In other embodiments, R^{4b1} is CH₃.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein R^{4b2} is H or C₁-C₆ alkyl. In other embodiments, R^{4b2} is H. In other embodiments, R^{4b2} is C₁-C₆ alkyl. In other embodiments, R^{4b2} is H or CH₃. In other embodiments, R^{4b2} is CH₃.

In some embodiments, the invention relates to a compound of any one of formulas (I), (I-A), (I-A-1), (I-A-2), (I-B), (I-B-1), (I-B-2), (I-C), (I-C-1), and (I-C-2), or a pharmaceutically acceptable salt thereof, wherein R^{5b1} is C₁-C₆ alkyl or C₁-C₆ haloalkyl. In other embodiments, R^{5b1} is C₁-C₆ alkyl. In other embodiments, R^{5b1} is C₁-C₆ haloalkyl. In other embodiments, R^{5b1} is CH₃ or CF₃. In other embodiments, R^{5b1} is CH₃. In other embodiments, R^{5b1} is CF₃.

In some embodiments, the invention relates to a compound of any one of formulas (I), (I-A), (I-A-1), (I-A-2), (I-B), (I-B-1), (I-B-2), (I-C), (I-C-1), and (I-C-2), or a pharmaceutically acceptable salt thereof, wherein R^{5b2} is C₁-C₆ alkyl or C₁-C₆ haloalkyl. In other embodiments, R^{5b2} is C₁-C₆ alkyl. In other embodiments, R^{5b2} is C₁-C₆ haloalkyl. In other embodiments, R^{5b2} is CH₃ or CF₃. In other embodiments, R^{5b2} is CH₃. In other embodiments, R^{5b2} is CF₃.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein R^{2c} is OH, halo, C₁-C₆ alkyl, C₁-C₆ alkoxy, or C₁-C₆ haloalkoxy. In other embodiments, R^{2c} is OH. In other embodiments, R^{2c} is halo. In other embodiments, R^{2c} is C₁-C₆ alkyl. In other embodiments, R^{2c} is C₁-C₆ alkoxy. In other embodiments, R^{2c} is C₁-C₆ haloalkoxy. In other embodiments, R^{2c} is OH, C₁, CH₃, OCH₃, OCD₃, OCH₂CH₃, OCH(CH₃)₂, OCHF₂, OCH₂CH₂F, or OCH₂CHF₂. In other embodiments, R^{2c} is C₁. In other embodiments, R^{2c} is CH₃. In other embodiments, R^{2c} is OCH₃. In other embodiments, R^{2c} is OCD₃. In other embodiments, R^{2c} is OCH₂CH₃. In other embodiments, R^{2c} is OCH(CH₃)₂. In other embodiments, R^{2c} is OCHF₂. In other embodiments, R^{2c} is OCH₂CH₂F. In other embodiments, R^{2c} is OCH₂CHF₂.

In some embodiments, the invention relates to a compound of any one of formulas (I), (I-A), (I-B), and (I-C), or a pharmaceutically acceptable salt thereof, wherein X^{3c} is N or C—R^{3c}; and R^{3c} is H, halo, C₁-C₆ alkyl, or C₁-C₆ haloalkyl. In other embodiments, X^{3c} is N. In other embodiments, X^{3c} is C—R^{3c}. In other embodiments, X^{3c} is C—R^{3c}; and R^{3c} is H. In other embodiments, X^{3c} is C—R^{3c}, and R^{3c} is halo. In other embodiments, X^{3c} is C—R^{3c}, and R^{3c} is C₁-C₆ alkyl. In other embodiments, X^{3c} is C—R^{3c}, and R^{3c} is C₁-C₆ haloalkyl. In other embodiments, X^{3c} is C—R^{3c}, and R^{3c} is H, F, CH₃, CHF₂, or CF₃. In other embodiments, X^{3c} is C—R^{3c}, and R^{3c} is F. In other embodiments, X^{3c} is C—R^{3c}, and R^{3c} is CH₃. In other embodiments, X^{3c} is C—R^{3c}, and R^{3c} is CHF₂. In other embodiments, X^{3c} is C—R^{3c}, and R^{3c} is CF₃.

In some embodiments, the invention relates to a compound of any one of formulas (I-A-1), (I-A-2), (I-A-3), (I-B-1), (I-B-2), (I-B-3), (I-C-1), (I-C-2), and (I-C-3), or a pharmaceutically acceptable salt thereof, wherein R^{3c} is H, halo, C₁-C₆ alkyl, or C₁-C₆ haloalkyl. In other embodiments, R^{3c} is H. In other embodiments, R^{3c} is halo. In other embodiments, R^{3c} is C₁-C₆ alkyl. In other embodiments, R^{3c} is C₁-C₆ haloalkyl. In other embodiments, R^{3c} is —(C₁-C₆ alkylene)-(C₁-C₆ alkoxy). In other embodiments, R^{3c} is H, F, CH₃, CHF₂, or CF₃. In other embodiments, R^{3c} is F. In other embodiments, R^{3c} is CH₃. In other embodiments, R^{3c} is CHF₂. In other embodiments, R^{3c} is CF₃.

In some embodiments, the invention relates to a compound of any one of formulas (I), (I-A), (I-B), and (I-C), or a pharmaceutically acceptable salt thereof, wherein X^{4c} is C—R^{4c}, and R^{4c} is H, halo, C₁-C₆ alkyl, or C₁-C₆ haloalkyl. In other embodiments, X^{4c} is C—R^{4c}. In other embodiments, X^{4c} is C—R^{4c}, and R^{4c} is H. In other embodiments, X^{4c} is C-R^{4c}, and R^{4c} is halo. In other embodiments, X^{4c} is C-R^{4c}; and R^{4c} is C₁-C₆ alkyl. In other embodiments, X^{4c} is C—R^{4c}, and R^{4c} is C₁-C₆ haloalkyl. In other embodiments, X^{4c} is C—R^{4c}, and R^{4c} is H, F, or CHF₂. In other embodiments, X^{4c} is C—R^{4c}, and R^{4c} is F. In other embodiments, X^{4c} is C—R^{4c}, and R^{4c} is CHF₂. In other embodiments, X^{4c} is C—R^{4c}, and R^{4c} is CH₂CH₃. In other embodiments, X^{4c} is C—R^{4c}, and R^{4c} is CHF₂. In other embodiments, X^{4c} is C—R^{4c}; and R^{4c} is CF₃.

In some embodiments, the invention relates to a compound of any one of formulas (I-A-1), (I-A-2), (I-A-3), (I-B-1), (I-B-2), (I-B-3), (I-C-1), (I-C-2), and (I-C-3), or a pharmaceutically acceptable salt thereof, wherein R^{4c} is H, halo, C₁-C₆ alkyl, or C₁-C₆ haloalkyl. In other embodiments, R^{4c} is H. In other embodiments, R^{4c} is halo. In other embodiments, R^{4c} is C₁-C₆ alkyl. In other embodiments, R^{4c} is C₁-C₆ haloalkyl. In other embodiments, R^{4c} is H, F, CHF₂, CH₂CH₃, CHF₂, CF₃. In other embodiments, R^{4c} is F. In other embodiments, R^{4c} is CHF₂. In other embodiments, R^{4c} is CH₂CH₃. In other embodiments, R^{4c} is CHF₂. In other embodiments, R^{4c} is CF₃.

In some embodiments, the invention relates to a compound of any one of formulas (I), (I-A), (I-B), and (I-C), or a pharmaceutically acceptable salt thereof, wherein X^{5c} is C-R^{5c}; and R^{5c} is H.

In some embodiments, the invention relates to a compound of any one of formulas (I), (I-A), (I-B), and (I-C), or a pharmaceutically acceptable salt thereof, wherein X^{6c} is C—R^{6c}; and R^{6c} is H.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or any embodiment thereof, i.e., the compound in non-salt form.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names.

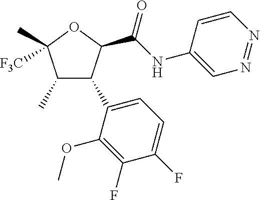 [SMILES: COc1c([C@H]2[C@H](C(=O)Nc3ccnnc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(pyridazin-4-
yl)-5-(trifluoromethyl)tetrahydrofuran-2-
carboxamide

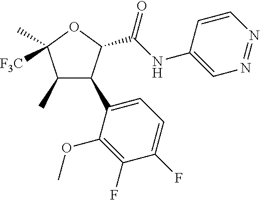 [SMILES: COc1c([C@@H]2[C@@H](C(=O)Nc3ccnnc3)O[C@](C)(C(F)(F)F)[C@@H]2C)ccc(F)c1F]

(2S,3R,4R,5S)-3-(3,4-difluoro-2-
methoxyphenyl)-4,5-dimethyl-N-(pyridazin-4-
yl)-5-(trifluoromethyl)tetrahydrofuran-2-
carboxamide

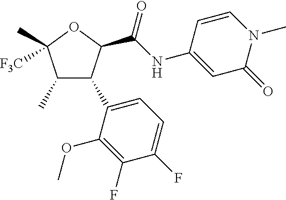

(2R,3S,4S,5R)-3-(3,4-difluoro-2-methoxyphenyl)-
4,5-dimethyl-N-(1-methyl-2-oxo-1,2-
dihydropyridin-4-yl)-5-
(trifluoromethyl)tetrahydrofuran-2-carboxamide

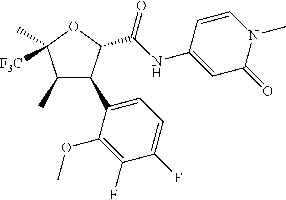

(2S,3R,4R,5S)-3-(3,4-difluoro-2-
methoxyphenyl)-4,5-dimethyl-N-(1-methyl-2-
oxo-1,2-dihydropyridin-4-yl)-5-
(trifluoromethyl)tetrahydrofuran-2-carboxamide

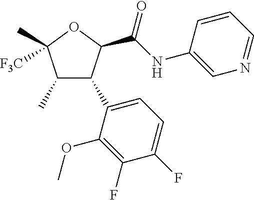 [SMILES: COc1c([C@H]2[C@H](C(=O)Nc3cccnc3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(pyridin-3-yl)-5-
(trifluoromethyl)tetrahydrofuran-2-carboxamide

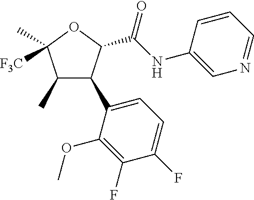 [SMILES: COc1c([C@@H]2[C@@H](C(=O)Nc3cccnc3)O[C@](C)(C(F)(F)F)[C@@H]2C)ccc(F)c1F]

(2S,3R,4R,5S)-3-(3,4-difluoro-2-
methoxyphenyl)-4,5-dimethyl-N-(pyridin-3-yl)-5-
(trifluoromethyl)tetrahydrofuran-2-carboxamide

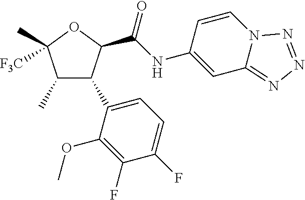 [SMILES: COc1c([C@H]2[C@H](C(=O)Nc3ccn4nnnc4c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(tetrazolo[1,5-a]pyridin-7-yl)-5-
(trifluoromethyl)tetrahydrofuran-2-carboxamide

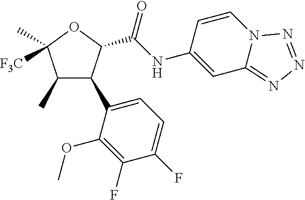 [SMILES: COc1c([C@@H]2[C@@H](C(=O)Nc3ccn4nnnc4c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(tetrazolo[1,5-a]pyridin-7-yl)-5-
(trifluoromethyl)tetrahydrofuran-2-carboxamide

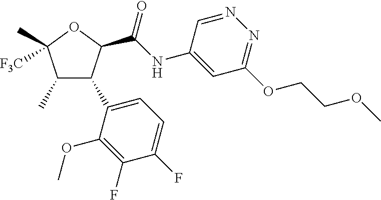

(2R,3S,4S,5R)-3-(3,4-difluoro-2-methoxyphenyl)-N-
(6-(2-methoxyethoxy)pyridazin-4-yl)-4,5-dimethyl-5-
(trifluoromethyl)tetrahydrofuran-2-carboxamide

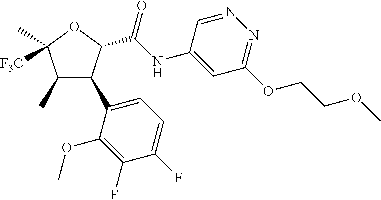

(2S,3R,4R,5S)-3-(3,4-difluoro-2-methoxyphenyl)-N-
(6-(2-methoxyethoxy)pyridazin-4-yl)-4,5-dimethyl-5-
(trifluoromethyl)tetrahydrofuran-2-carboxamide

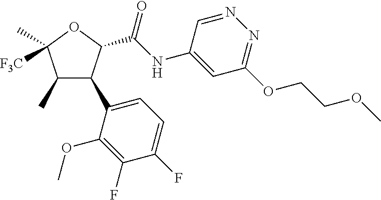

(2S,3R,4R,5S)-3-(3,4-difluoro-2-methoxyphenyl)-N-
(6-(2-methoxyethoxy)pyridazin-4-yl)-4,5-dimethyl-5-
(trifluoromethyl)tetrahydrofuran-2-carboxamide

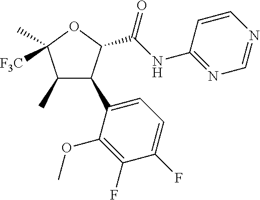 [SMILES: COc1c([C@@H]2[C@@H](C(=O)Nc3ccncn3)O[C@](C)(C(F)(F)F)[C@@H]2C)ccc(F)c1F]

(2S,3R,4R,5S)-3-(3,4-difluoro-2-methoxyphenyl)-
4,5-dimethyl-N-(pyrimidin-4-yl)-5-
(trifluoromethyl)tetrahydrofuran-2-carboxamide

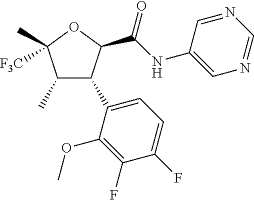 [SMILES: COc1c([C@H]2[C@H](C(=O)Nc3cncnc3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(pyrimidin-5-yl)-5-
(trifluoromethyl)tetrahydrofuran-2-carboxamide

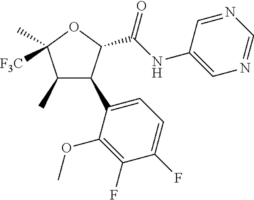 [SMILES: COc1c([C@@H]2[C@@H](C(=O)Nc3cncnc3)O[C@](C)(C(F)(F)F)[C@@H]2C)ccc(F)c1F]

(2S,3R,4R,5S)-3-(3,4-difluoro-2-methoxyphenyl)-
4,5-dimethyl-N-(pyrimidin-5-yl)-5-
(trifluoromethyl)tetrahydrofuran-2-carboxamide

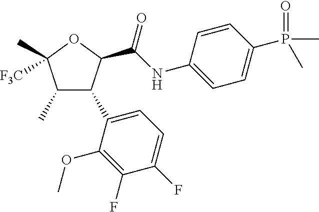

(2R,3S,4S,5R)-3-(3,4-difluoro-2-methoxyphenyl)-N-(4-
(dimethylphosphoryl)phenyl)-4,5-dimethyl-5-
(trifluoromethyl)tetrahydrofuran-2-carboxamide

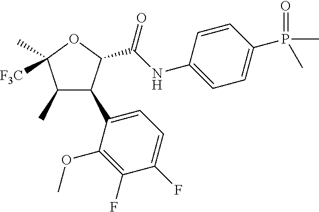

(2S,3R,4R,5S)-3-(3,4-difluoro-2-methoxyphenyl)-N-(4-
(dimethylphosphoryl)phenyl)-4,5-dimethyl-5-
(trifluoromethyl)tetrahydrofuran-2-carboxamide

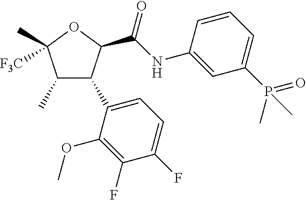 [SMILES: COc1c([C@H]2[C@H](C(=O)Nc3cccc(P(C)(C)=O)c3)O[C@@](C)(C(F)(F)F)[C@H]2C)ccc(F)c1F]

(2R,3S,4S,5R)-3-(3,4-difluoro-2-methoxyphenyl)-N-(3-
(dimethylphosphoryl)phenyl)-4,5-dimethyl-5-
(trifluoromethyl)tetrahydrofuran-2-carboxamide

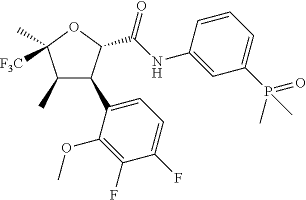 [SMILES: COc1c([C@@H]2[C@@H](C(=O)Nc3cccc(P(C)(C)=O)c3)O[C@](C)(C(F)(F)F)[C@@H]2C)ccc(F)c1F]

(2S,3R,4R,5S)-3-(3,4-difluoro-2-methoxyphenyl)-N-(3-
(dimethylphosphoryl)phenyl)-4,5-dimethyl-5-
(trifluoromethyl)tetrahydrofuran-2-carboxamide

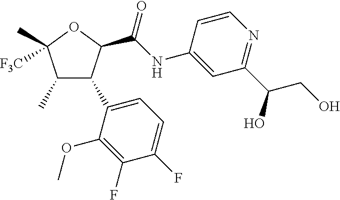 [SMILES: COc1c([C@H]2[C@H](C(=O)Nc3ccnc([C@H](O)CO)c3)O[C@@](C)(C(F)(F)F)[C@H]2C)ccc(F)c1F]

(2R,3S,4S,5R)-3-(3,4-difluoro-2-
methoxyphenyl)-N-(2-((S)-1,2-
dihydroxyethyl)pyridin-4-yl)-4,5-dimethyl-5-
(trifluoromethyl)tetrahydrofuran-2-carboxamide

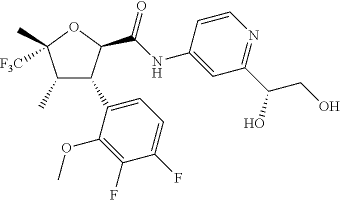 [SMILES: COc1c([C@H]2[C@H](C(=O)Nc3ccnc([C@@H](O)CO)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1,2-dihydroxyethyl)pyridin-4-yl)-4,5-
dimethyl-5-(trifluoromethyl)tetrahydrofuran-2-
carboxamide

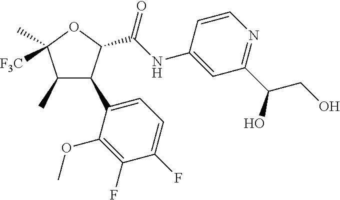 [SMILES: COc1c([C@@H]2[C@@H](C(=O)Nc3ccnc([C@H](O)CO)c3)O[C@](C)(C(F)(F)F)[C@@H]2C)ccc(F)c1F]

(2S,3R,4R,5S)-3-(3,4-difluoro-2-
methoxyphenyl)-N-(2-((S)-1,2-
dihydroxyethyl)pyridin-4-yl)-4,5-dimethyl-5-
(trifluoromethyl)tetrahydrofuran-2-carboxamide

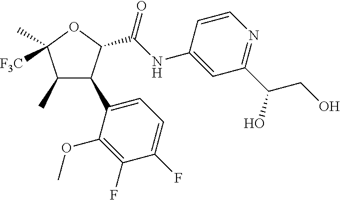 [SMILES: COc1c([C@@H]2[C@@H](C(=O)Nc3ccnc([C@@H](O)CO)c3)O[C@](C)(C(F)(F)F)[C@@H]2C)ccc(F)c1F]

(2S,3R,4R,5S)-3-(3,4-difluoro-2-
methoxyphenyl)-N-(2-((R)-1,2-
dihydroxyethyl)pyridin-4-yl)-4,5-dimethyl-5-
(trifluoromethyl)tetrahydrofuran-2-carboxamide

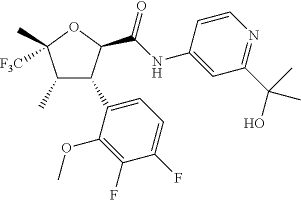 [SMILES: COc1c([C@H]2[C@H](C(=O)Nc3ccnc(C(C)(C)O)c3)O[C@@](C)(C(F)(F)F)[C@H]2C)ccc(F)c1F]

(2R,3S,4S,5R)-3-(3,4-difluoro-2-methoxyphenyl)-N-
(2-(2-hydroxypropan-2-yl)pyridin-4-yl)-4,5-dimethyl-5-
(trifluoromethyl)tetrahydrofuran-2-carboxamide

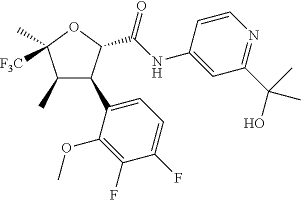 [SMILES: COc1c([C@@H]2[C@@H](C(=O)Nc3ccnc(C(C)(C)O)c3)O[C@](C)(C(F)(F)F)[C@@H]2C)ccc(F)c1F]

(2S,3R,4R,5S)-3-(3,4-difluoro-2-methoxyphenyl)-N-
(2-(2-hydroxypropan-2-yl)pyridin-4-yl)-4,5-dimethyl-5-
(trifluoromethyl)tetrahydrofuran-2-carboxamide

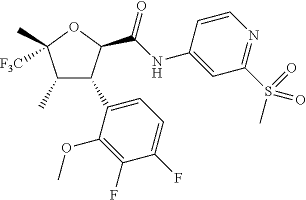 [SMILES: COc1c([C@H]2[C@H](C(=O)Nc3ccnc(S(C)(=O)=O)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(methylsulfonyl)pyridin-4-yl)-5-
(trifluoromethyl)tetrahydrofuran-2-carboxamide

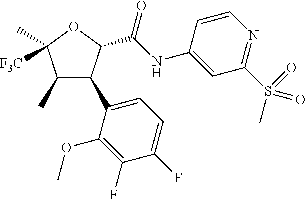 [SMILES: COc1c([C@@H]2[C@@H](C(=O)Nc3ccnc(S(C)(=O)=O)c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(2-(methylsulfonyl)pyridin-4-yl)-5-
(trifluoromethyl)tetrahydrofuran-2-carboxamide

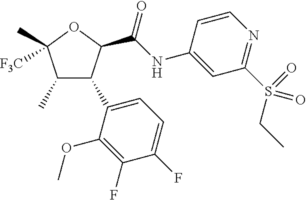 [SMILES: CCS(=O)(=O)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-N-
(2-(ethylsulfonyl)pyridin-4-yl)-4,5-dimethyl-5-
(trifluoromethyl)tetrahydrofuran-2-carboxamide

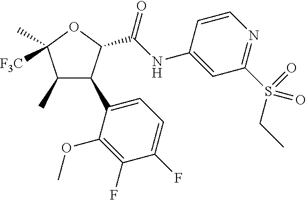 [SMILES: CCS(=O)(=O)c1cc(NC(=O)[C@H]2O[C@](C)(C(F)(F)F)[C@H](C)[C@@H]2c2ccc(F)c(F)c2OC)ccn1]

(2S,3R,4R,5S)-3-(3,4-difluoro-2-methoxyphenyl)-N-
(2-(ethylsulfonyl)pyridin-4-yl)-4,5-dimethyl-5-
(trifluoromethyl)tetrahydrofuran-2-carboxamide

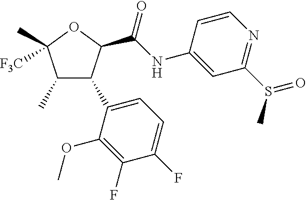 [SMILES: COc1c([C@H]2[C@H](C(=O)Nc3ccnc([S@](C)=O)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-((S)-methylsulfinyl)pyridin-4-yl)-5-
(trifluoromethyl)tetrahydrofuran-2-carboxamide

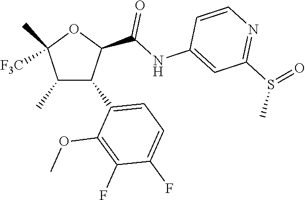 [SMILES: COc1c([C@H]2[C@H](C(=O)Nc3ccnc([S@@](C)=O)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-((R)-methylsulfinyl)pyridin-4-yl)-5-
(trifluoromethyl)tetrahydrofuran-2-carboxamide

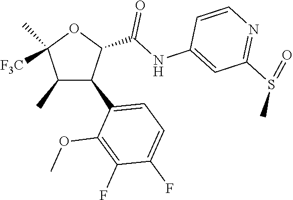 [SMILES: COc1c([C@@H]2[C@@H](C(=O)Nc3ccnc([S@](C)=O)c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(2-((S)-methylsulfinyl)pyridin-4-yl)-5-
(trifluoromethyl)tetrahydrofuran-2-carboxamide

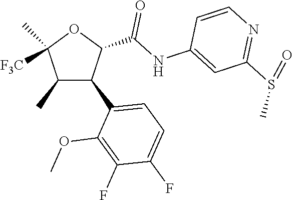 [SMILES: COc1c([C@@H]2[C@@H](C(=O)Nc3ccnc([S@@](C)=O)c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(2-((R)-methylsulfinyl)pyridin-4-yl)-5-
(trifluoromethyl)tetrahydrofuran-2-carboxamide

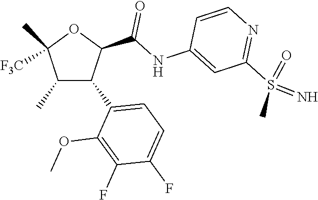 [SMILES: COc1c([C@H]2[C@H](C(=O)Nc3ccnc([S@@](C)(=N)=O)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(S-methylsulfonimidoyl)pyridin-4-yl)-5-
(trifluoromethyl)tetrahydrofuran-2-carboxamide

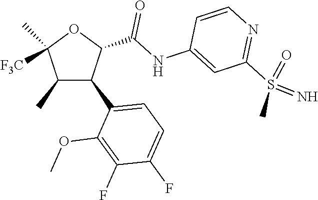 [SMILES: COc1c([C@@H]2[C@@H](C(=O)Nc3ccnc([S@@](C)(=N)=O)c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(2-(S-methylsulfonimidoyl)pyridin-4-yl)-5-
(trifluoromethyl)tetrahydrofuran-2-carboxamide

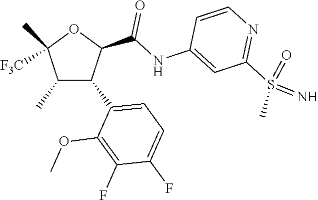 [SMILES: COc1c([C@H]2[C@H](C(=O)Nc3ccnc([S@](C)(=N)=O)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(S-methylsulfonimidoyl)pyridin-4-yl)-5-
(trifluoromethyl)tetrahydrofuran-2-carboxamide

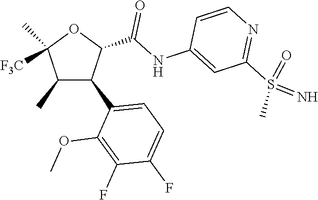 [SMILES: COc1c([C@@H]2[C@@H](C(=O)Nc3ccnc([S@](C)(=N)=O)c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(2-(S-methylsulfonimidoyl)pyridin-4-yl)-5-
(trifluoromethyl)tetrahydrofuran-2-carboxamide

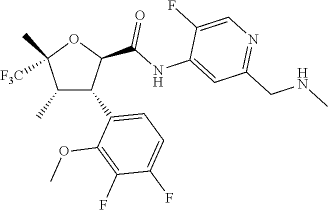 [SMILES: CNCc1cc(NC(=O)[C@@H]2O[C@@](C)(C(F)(F)F)[C@@H](C)[C@H]2c2ccc(F)c(F)c2OC)c(F)cn1]

(2R,3S,4S,5R)-3-(3,4-difluoro-2-methoxyphenyl)-
N-(5-fluoro-2-((methylamino)methyl)pyridin-4-yl)-
4,5-dimethyl-5-(trifluoromethyl)tetrahydrofuran-2-
carboxamide

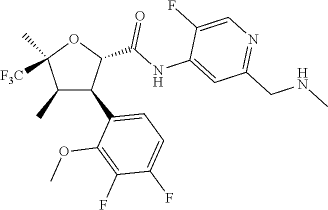 [SMILES: CNCc1cc(NC(=O)[C@H]2O[C@](C)(C(F)(F)F)[C@H](C)[C@@H]2c2ccc(F)c(F)c2OC)c(F)cn1]

(2S,3R,4R,5S)-3-(3,4-difluoro-2-methoxyphenyl)-N-
(5-fluoro-2-((methylamino)methyl)pyridin-4-yl)-4,5-
dimethyl-5-(trifluoromethyl)tetrahydrofuran-2-
carboxamide

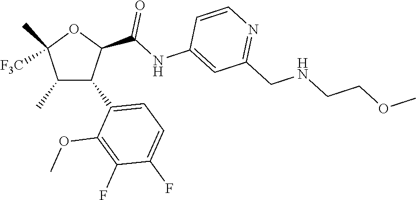 [SMILES: COCCNCc1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-(((2-methoxyethyl)amino)methyl)pyridin-4-
yl)-4,5-dimethyl-5-(trifluoromethyl)tetrahydrofuran-
2-carboxamide

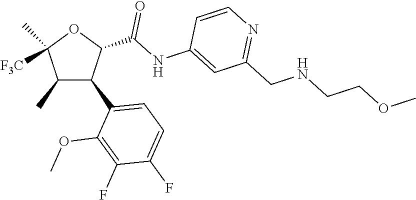 [SMILES: COCCNCc1cc(NC(=O)[C@H]2O[C@](C)(C(F)(F)F)[C@H](C)[C@@H]2c2ccc(F)c(F)c2OC)ccn1]

(2S,3R,4R,5S)-3-(3,4-difluoro-2-methoxyphenyl)-N-
(2-(((2-methoxyethyl)amino)methyl)pyridin-4-yl)-
4,5-dimethyl-5-(trifluoromethyl)tetrahydrofuran-2-
carboxamide

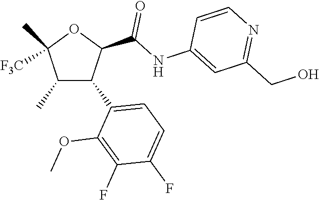 [SMILES: COc1c([C@H]2[C@H](C(=O)Nc3ccnc(CO)c3)O[C@@](C)(C(F)(F)F)[C@H]2C)ccc(F)c1F]

(2R,3S,4S,5R)-3-(3,4-difluoro-2-methoxyphenyl)-
N-(2-(hydroxymethyl)pyridin-4-yl)-4,5-dimethyl-5-
(trifluoromethyl)tetrahydrofuran-2-carboxamide

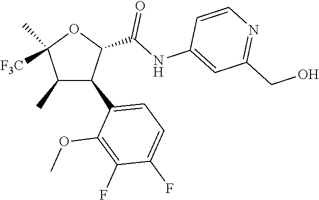 [SMILES: COc1c([C@@H]2[C@@H](C(=O)Nc3ccnc(CO)c3)O[C@](C)(C(F)(F)F)[C@@H]2C)ccc(F)c1F]

(2S,3R,4R,5S)-3-(3,4-difluoro-2-methoxyphenyl)-N-
(2-(hydroxymethyl)pyridin-4-yl)-4,5-dimethyl-5-
(trifluoromethyl)tetrahydrofuran-2-carboxamide

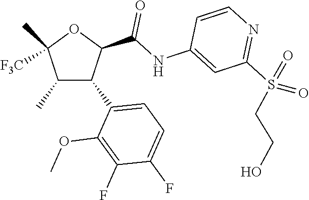 [SMILES: COc1c([C@H]2[C@H](C(=O)Nc3ccnc(S(=O)(=O)CCO)c3)O[C@@](C)(C(F)(F)F)[C@H]2C)ccc(F)c1F]

(2R,3S,4S,5R)-3-(3,4-difluoro-2-methoxyphenyl)-N-
(2-((2-hydroxyethyl)sulfonyl)pyridin-4-yl)-4,5-
dimethyl-5-(trifluoromethyl)tetrahydrofuran-2-
carboxamide

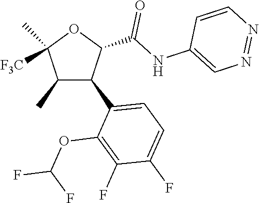 [SMILES: C[C@@H]1[C@H](c2ccc(F)c(F)c2OC(F)F)[C@@H](C(=O)Nc2ccnnc2)O[C@]1(C)C(F)(F)F]

(2S,3R,4R,5S)-3-(2-(difluoromethoxy)-3,4-
difluorophenyl)-4,5-dimethyl-N-(pyridazin-4-yl)-
5-(trifluoromethyl)tetrahydrofuran-2-
carboxamide

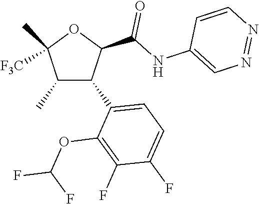 [SMILES: C[C@H]1[C@@H](c2ccc(F)c(F)c2OC(F)F)[C@H](C(=O)Nc2ccnnc2)O[C@@]1(C)C(F)(F)F]

(2R,3S,4S,5R)-3-(2-(difluoromethoxy)-3,4-
difluorophenyl)-4,5-dimethyl-N-(pyridazin-4-yl)-
5-(trifluoromethyl)tetrahydrofuran-2-
carboxamide

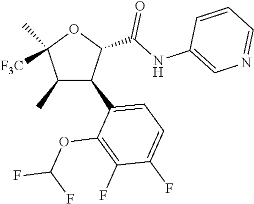 [SMILES: C[C@@H]1[C@H](c2ccc(F)c(F)c2OC(F)F)[C@@H](C(=O)Nc2cccnc2)O[C@]1(C)C(F)(F)F]

(2S,3R,4R,5S)-3-(2-(difluoromethoxy)-3,4-
difluorophenyl)-4,5-dimethyl-N-(pyridin-3-yl)-5-
(trifluoromethyl)tetrahydrofuran-2-carboxamide

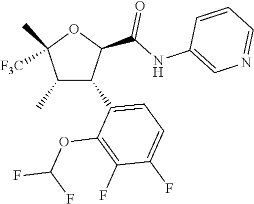 [SMILES: C[C@H]1[C@@H](c2ccc(F)c(F)c2OC(F)F)[C@H](C(=O)Nc2cccnc2)O[C@@]1(C)C(F)(F)F]

(2R,3S,4S,5R)-3-(2-(difluoromethoxy)-3,4-
difluorophenyl)-4,5-dimethyl-N-(pyridin-3-yl)-5-
(trifluoromethyl)tetrahydrofuran-2-carboxamide

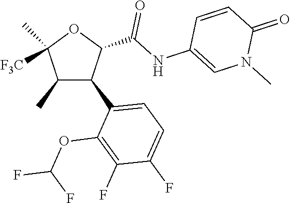 [SMILES: C[C@@H]1[C@H](c2ccc(F)c(F)c2OC(F)F)[C@@H](C(=O)Nc2ccc(=O)n(C)c2)O[C@]1(C)C(F)(F)F]

(2S,3R,4R,5S)-3-(2-(difluoromethoxy)-3,4-
difluorophenyl)-4,5-dimethyl-N-(1-methyl-6-oxo-
1,6-dihydropyridin-3-yl)-5-
(trifluoromethyl)tetrahydrofuran-2-carboxamide

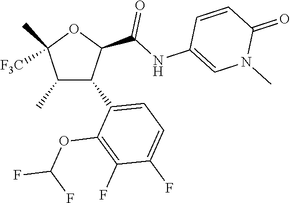 [SMILES: C[C@H]1[C@@H](c2ccc(F)c(F)c2OC(F)F)[C@H](C(=O)Nc2ccc(=O)n(C)c2)O[C@@]1(C)C(F)(F)F]

(2R,3S,4S,5R)-3-(2-(difluoromethoxy)-3,4-
difluorophenyl)-4,5-dimethyl-N-(1-methyl-6-oxo-
1,6-dihydropyridin-3-yl)-5-
(trifluoromethyl)tetrahydrofuran-2-carboxamide

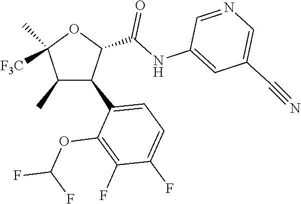 [SMILES: C[C@@H]1[C@H](c2ccc(F)c(F)c2OC(F)F)[C@@H](C(=O)Nc2cncc(C#N)c2)O[C@]1(C)C(F)(F)F]

(2S,3R,4R,5S)-N-(5-cyanopyridin-3-yl)-3-(2-
(difluoromethoxy)-3,4-difluorophenyl)-4,5-
dimethyl-5-(trifluoromethyl)tetrahydrofuran-2-
carboxamide

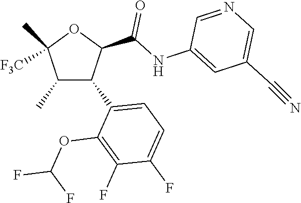 [SMILES: C[C@H]1[C@@H](c2ccc(F)c(F)c2OC(F)F)[C@H](C(=O)Nc2cncc(C#N)c2)O[C@@]1(C)C(F)(F)F]

(2R,3S,4S,5R)-N-(5-cyanopyridin-3-yl)-3-(2-
(difluoromethoxy)-3,4-difluorophenyl)-4,5-
dimethyl-5-(trifluoromethyl)tetrahydrofuran-2-
carboxamide

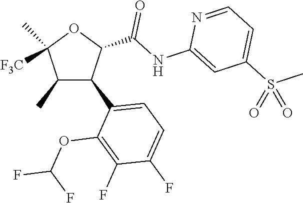 [SMILES: C[C@@H]1[C@H](c2ccc(F)c(F)c2OC(F)F)[C@@H](C(=O)Nc2cc(S(C)(=O)=O)ccn2)O[C@]1(C)C(F)(F)F]

(2S,3R,4R,5S)-3-(2-(difluoromethoxy)-3,4-
difluorophenyl)-4,5-dimethyl-N-(4-
(methylsulfonyl)pyridin-2-yl)-5-
(trifluoromethyl)tetrahydrofuran-2-carboxamide

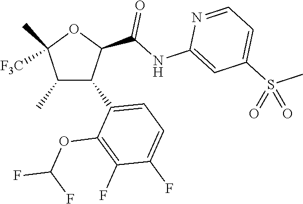 [SMILES: C[C@H]1[C@@H](c2ccc(F)c(F)c2OC(F)F)[C@H](C(=O)Nc2cc(S(C)(=O)=O)ccn2)O[C@@]1(C)C(F)(F)F]

(2R,3S,4S,5R)-3-(2-(difluoromethoxy)-3,4-
difluorophenyl)-4,5-dimethyl-N-(4-
(methylsulfonyl)pyridin-2-yl)-5-
(trifluoromethyl)tetrahydrofuran-2-carboxamide

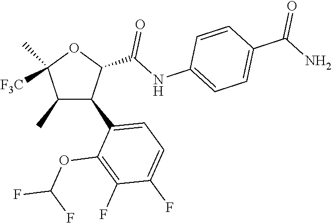 [SMILES: C[C@@H]1[C@H](c2ccc(F)c(F)c2OC(F)F)[C@@H](C(=O)Nc2ccc(C(N)=O)cc2)O[C@]1(C)C(F)(F)F]

(2S,3R,4R,5S)-N-(4-carbamoylphenyl)-3-(2-
(difluoromethoxy)-3,4-difluorophenyl)-4,5-
dimethyl-5-(trifluoromethyl)tetrahydrofuran-2-
carboxamide

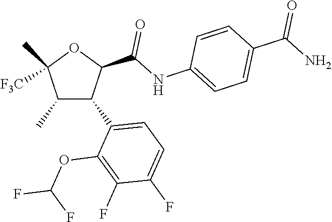 [SMILES: C[C@H]1[C@@H](c2ccc(F)c(F)c2OC(F)F)[C@H](C(=O)Nc2ccc(C(N)=O)cc2)O[C@@]1(C)C(F)(F)F]

(2R,3S,4S,5R)-N-(4-carbamoylphenyl)-3-(2-
(difluoromethoxy)-3,4-difluorophenyl)-4,5-
dimethyl-5-(trifluoromethyl)tetrahydrofuran-2-
carboxamide

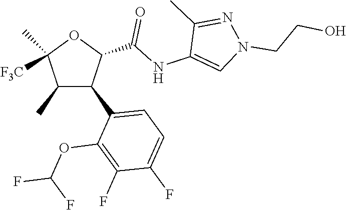 [SMILES: Cc1nn(CCO)cc1NC(=O)[C@H]1O[C@](C)(C(F)(F)F)[C@H](C)[C@@H]1c1ccc(F)c(F)c1OC(F)F]

(2S,3R,4R,5S)-3-(2-(difluoromethoxy)-3,4-
difluorophenyl)-N-(1-(2-hydroxyethyl)-3-methyl-
1H-pyrazol-4-yl)-4,5-dimethyl-5-
(trifluoromethyl)tetrahydrofuran-2-carboxamide

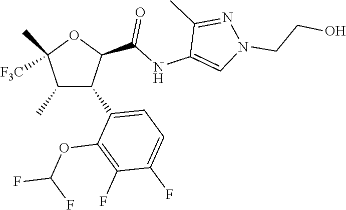 [SMILES: Cc1nn(CCO)cc1NC(=O)[C@@H]1O[C@@](C)(C(F)(F)F)[C@@H](C)[C@H]1c1ccc(F)c(F)c1OC(F)F]

(2R,3S,4S,5R)-3-(2-(difluoromethoxy)-3,4-
difluorophenyl)-N-(1-(2-hydroxyethyl)-3-methyl-
1H-pyrazol-4-yl)-4,5-dimethyl-5-
(trifluoromethyl)tetrahydrofuran-2-carboxamide

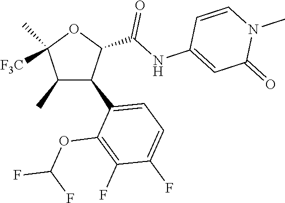 [SMILES: C[C@@H]1[C@H](c2ccc(F)c(F)c2OC(F)F)[C@@H](C(=O)Nc2ccn(C)c(=O)c2)O[C@]1(C)C(F)(F)F]

(2S,3R,4R,5S)-3-(2-(difluoromethoxy)-3,4-
difluorophenyl)-4,5-dimethyl-N-(1-methyl-2-oxo-
1,2-dihydropyridin-4-yl)-5-
(trifluoromethyl)tetrahydrofuran-2-carboxamide

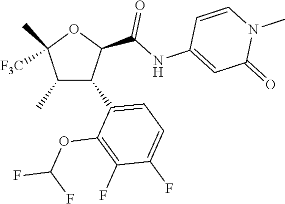 [SMILES: C[C@H]1[C@@H](c2ccc(F)c(F)c2OC(F)F)[C@H](C(=O)Nc2ccn(C)c(=O)c2)O[C@@]1(C)C(F)(F)F]

(2R,3S,4S,5R)-3-(2-(difluoromethoxy)-3,4-
difluorophenyl)-4,5-dimethyl-N-(1-methyl-2-oxo-
1,2-dihydropyridin-4-yl)-5-
(trifluoromethyl)tetrahydrofuran-2-carboxamide

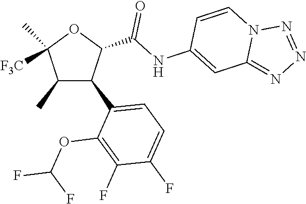 [SMILES: C[C@@H]1[C@H](c2ccc(F)c(F)c2OC(F)F)[C@@H](C(=O)Nc2ccn3nnnc3c2)O[C@]1(C)C(F)(F)F]

(2S,3R,4R,5S)-3-(2-(difluoromethoxy)-3,4-
difluorophenyl)-4,5-dimethyl-N-(tetrazolo[1,5-
a]pyridin-7-yl)-5-
(trifluoromethyl)tetrahydrofuran-2-carboxamide

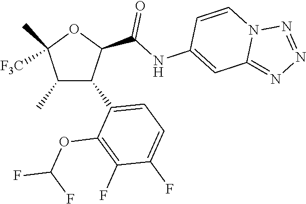 [SMILES: C[C@H]1[C@@H](c2ccc(F)c(F)c2OC(F)F)[C@H](C(=O)Nc2ccn3nnnc3c2)O[C@@]1(C)C(F)(F)F]

(2R,3S,4S,5R)-3-(2-(difluoromethoxy)-3,4-
difluorophenyl)-4,5-dimethyl-N-(tetrazolo[1,5-
a]pyridin-7-yl)-5-
(trifluoromethyl)tetrahydrofuran-2-carboxamide

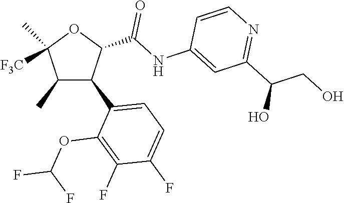 [SMILES: C[C@@H]1[C@H](c2ccc(F)c(F)c2OC(F)F)[C@@H](C(=O)Nc2ccnc([C@H](O)CO)c2)O[C@]1(C)C(F)(F)F]

(2S,3R,4R,5S)-3-(2-(difluoromethoxy)-3,4-
difluorophenyl)-N-(2-((S)-1,2-
dihydroxyethyl)pyridin-4-yl)-4,5-dimethyl-5-
(trifluoromethyl)tetrahydrofuran-2-carboxamide

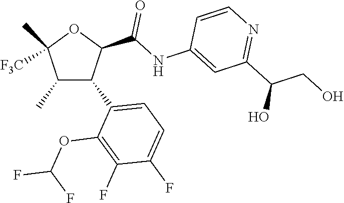 [SMILES: C[C@H]1[C@@H](c2ccc(F)c(F)c2OC(F)F)[C@H](C(=O)Nc2ccnc([C@H](O)CO)c2)O[C@@]1(C)C(F)(F)F]

(2R,3S,4S,5R)-3-(2-(difluoromethoxy)-3,4-
difluorophenyl)-N-(2-((S)-1,2-
dihydroxyethyl)pyridin-4-yl)-4,5-dimethyl-5-
(trifluoromethyl)tetrahydrofuran-2-carboxamide

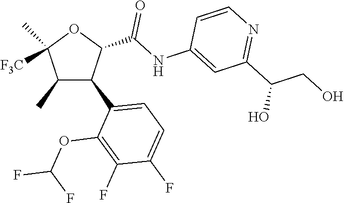 [SMILES: C[C@@H]1[C@H](c2ccc(F)c(F)c2OC(F)F)[C@@H](C(=O)Nc2ccnc([C@@H](O)CO)c2)O[C@]1(C)C(F)(F)F]

(2S,3R,4R,5S)-3-(2-(difluoromethoxy)-3,4-
difluorophenyl)-N-(2-((R)-1,2-
dihydroxyethyl)pyridin-4-yl)-4,5-dimethyl-5-
(trifluoromethyl)tetrahydrofuran-2-carboxamide

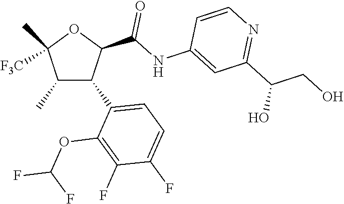 [SMILES: C[C@H]1[C@@H](c2ccc(F)c(F)c2OC(F)F)[C@H](C(=O)Nc2ccnc([C@@H](O)CO)c2)O[C@@]1(C)C(F)(F)F]

(2R,3S,4S,5R)-3-(2-(difluoromethoxy)-3,4-
difluorophenyl)-N-(2-((R)-1,2-
dihydroxyethyl)pyridin-4-yl)-4,5-dimethyl-5-
(trifluoromethyl)tetrahydrofuran-2-carboxamide

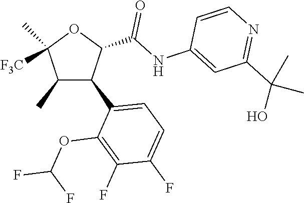 [SMILES: C[C@@H]1[C@H](c2ccc(F)c(F)c2OC(F)F)[C@@H](C(=O)Nc2ccnc(C(C)(C)O)c2)O[C@]1(C)C(F)(F)F]

(2S,3R,4R,5S)-3-(2-(difluoromethoxy)-3,4-
difluorophenyl)-N-(2-(2-hydroxypropan-2-
yl)pyridin-4-yl)-4,5-dimethyl-5-
(trifluoromethyl)tetrahydrofuran-2-carboxamide

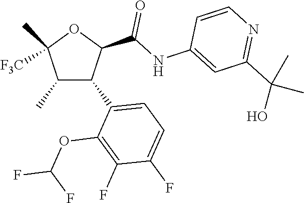 [SMILES: C[C@H]1[C@@H](c2ccc(F)c(F)c2OC(F)F)[C@H](C(=O)Nc2ccnc(C(C)(C)O)c2)O[C@@]1(C)C(F)(F)F]

(2R,3S,4S,5R)-3-(2-(difluoromethoxy)-3,4-
difluorophenyl)-N-(2-(2-hydroxypropan-2-
yl)pyridin-4-yl)-4,5-dimethyl-5-
(trifluoromethyl)tetrahydrofuran-2-carboxamide

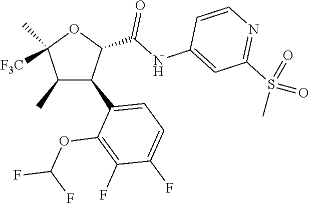 [SMILES: C[C@@H]1[C@H](c2ccc(F)c(F)c2OC(F)F)[C@@H](C(=O)Nc2ccnc(S(C)(=O)=O)c2)O[C@]1(C)C(F)(F)F]

(2S,3R,4R,5S)-3-(2-(difluoromethoxy)-3,4-
difluorophenyl)-4,5-dimethyl-N-(2-
(methylsulfonyl)pyridin-4-yl)-5-
(trifluoromethyl)tetrahydrofuran-2-carboxamide

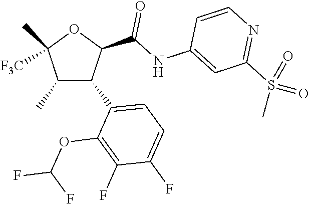 [SMILES: C[C@H]1[C@@H](c2ccc(F)c(F)c2OC(F)F)[C@H](C(=O)Nc2ccnc(S(C)(=O)=O)c2)O[C@@]1(C)C(F)(F)F]

(2R,3S,4S,5R)-3-(2-(difluoromethoxy)-3,4-
difluorophenyl)-4,5-dimethyl-N-(2-
(methylsulfonyl)pyridin-4-yl)-5-
(trifluoromethyl)tetrahydrofuran-2-carboxamide

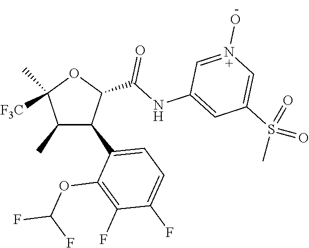

3-((2S,3R,4R,5S)-3-(2-(difluoromethoxy)-3,4-
difluorophenyl)-4,5-dimethyl-5-
(trifluoromethyl)tetrahydrofuran-2-carboxamido)-
5-(methylsulfonyl)pyridine 1-oxide

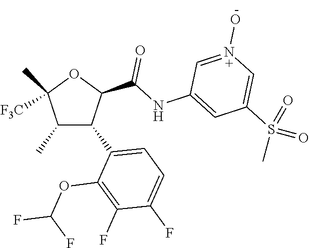

3-((2R,3S,4S,5R)-3-(2-(difluoromethoxy)-3,4-
difluorophenyl)-4,5-dimethyl-5-
(trifluoromethyl)tetrahydrofuran-2-carboxamido)-5-
(methylsulfonyl)pyridine 1-oxide

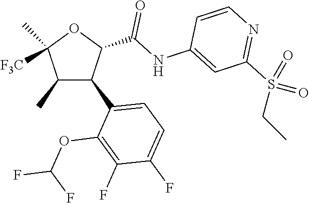 [SMILES: CCS(=O)(=O)c1cc(NC(=O)[C@H]2O[C@](C)(C(F)(F)F)[C@H](C)[C@@H]2c2ccc(F)c(F)c2OC(F)F)ccn1]

(2S,3R,4R,5S)-3-(2-(difluoromethoxy)-3,4-
difluorophenyl)-N-(2-(ethylsulfonyl)pyridin-4-yl)-
4,5-dimethyl-5-(trifluoromethyl)tetrahydrofuran-
2-carboxamide

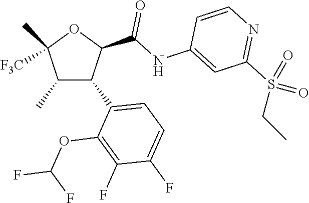 [SMILES: CCS(=O)(=O)c1cc(NC(=O)[C@@H]2O[C@@](C)(C(F)(F)F)[C@@H](C)[C@H]2c2ccc(F)c(F)c2OC(F)F)ccn1]

(2R,3S,4S,5R)-3-(2-(difluoromethoxy)-3,4-
difluorophenyl)-N-(2-(ethylsulfonyl)pyridin-4-yl)-
4,5-dimethyl-5-(trifluoromethyl)tetrahydrofuran-
2-carboxamide

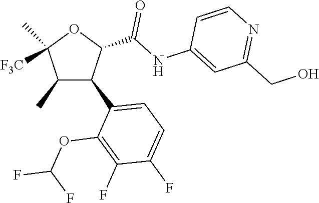 [SMILES: C[C@@H]1[C@H](c2ccc(F)c(F)c2OC(F)F)[C@@H](C(=O)Nc2ccnc(CO)c2)O[C@]1(C)C(F)(F)F]

(2S,3R,4R,5S)-3-(2-(difluoromethoxy)-3,4-
difluorophenyl)-N-(2-(hydroxymethyl)pyridin-4-
yl)-4,5-dimethyl-5-
(trifluoromethyl)tetrahydrofuran-2-carboxamide

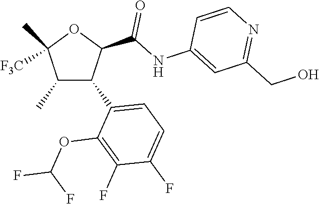 [SMILES: C[C@H]1[C@@H](c2ccc(F)c(F)c2OC(F)F)[C@H](C(=O)Nc2ccnc(CO)c2)O[C@@]1(C)C(F)(F)F]

(2R,3S,4S,5R)-3-(2-(difluoromethoxy)-3,4-
difluorophenyl)-N-(2-(hydroxymethyl)pyridin-4-
yl)-4,5-dimethyl-5-
(trifluoromethyl)tetrahydrofuran-2-carboxamide

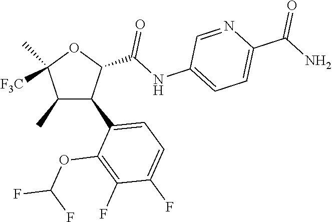 [SMILES: C[C@@H]1[C@H](c2ccc(F)c(F)c2OC(F)F)[C@@H](C(=O)Nc2ccc(C(N)=O)nc2)O[C@]1(C)C(F)(F)F]

5-((2S,3R,4R,5S)-3-(2-(difluoromethoxy)-3,4-
difluorophenyl)-4,5-dimethyl-5-
(trifluoromethyl)tetrahydrofuran-2-
carboxamido)picolinamide

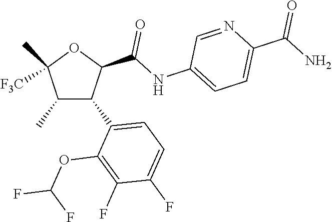 [SMILES: C[C@H]1[C@@H](c2ccc(F)c(F)c2OC(F)F)[C@H](C(=O)Nc2ccc(C(N)=O)nc2)O[C@@]1(C)C(F)(F)F]

5-((2R,3S,4S,5R)-3-(2-(difluoromethoxy)-3,4-
difluorophenyl)-4,5-dimethyl-5-
(trifluoromethyl)tetrahydrofuran-2-
carboxamido)picolinamide

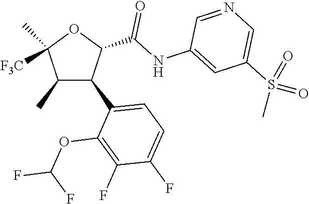 [SMILES: C[C@@H]1[C@H](c2ccc(F)c(F)c2OC(F)F)[C@@H](C(=O)Nc2cncc(S(C)(=O)=O)c2)O[C@]1(C)C(F)(F)F]

(2S,3R,4R,5S)-3-(2-(difluoromethoxy)-3,4-
difluorophenyl)-4,5-dimethyl-N-(5-
(methylsulfonyl)pyridin-3-yl)-5-
(trifluoromethyl)tetrahydrofuran-2-carboxamide

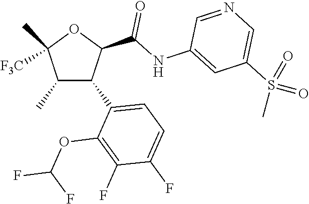 [SMILES: C[C@H]1[C@@H](c2ccc(F)c(F)c2OC(F)F)[C@H](C(=O)Nc2cncc(S(C)(=O)=O)c2)O[C@@]1(C)C(F)(F)F]

(2R,3S,4S,5R)-3-(2-(difluoromethoxy)-3,4-
difluorophenyl)-4,5-dimethyl-N-(5-
(methylsulfonyl)pyridin-3-yl)-5-
(trifluoromethyl)tetrahydrofuran-2-carboxamide

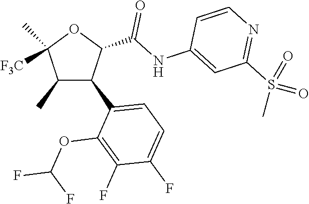 [SMILES: C[C@@H]1[C@H](c2ccc(F)c(F)c2OC(F)F)[C@@H](C(=O)Nc2ccnc(S(C)(=O)=O)c2)O[C@]1(C)C(F)(F)F]

(2S,3R,4R,5S)-3-(2-(difluoromethoxy)-4-fluoro-3-
methylphenyl)-4,5-dimethyl-N-(2-
(methylsulfonyl)pyridin-4-yl)-5-
(trifluoromethyl)tetrahydrofuran-2-carboxamide

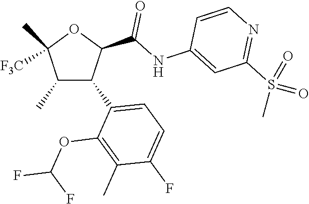 [SMILES: Cc1c(F)ccc([C@H]2[C@H](C(=O)Nc3ccnc(S(C)(=O)=O)c3)O[C@@](C)(C(F)(F)F)[C@H]2C)c1OC(F)F]

(2R,3S,4S,5R)-3-(2-(difluoromethoxy)-4-fluoro-3-
methylphenyl)-4,5-dimethyl-N-(2-
(methylsulfonyl)pyridin-4-yl)-5-
(trifluoromethyl)tetrahydrofuran-2-carboxamide

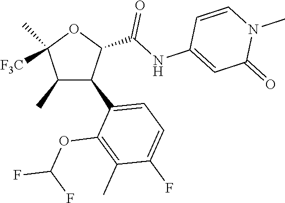 [SMILES: Cc1c(F)ccc([C@@H]2[C@@H](C(=O)Nc3ccn(C)c(=O)c3)O[C@](C)(C(F)(F)F)[C@@H]2C)c1OC(F)F]

(2S,3R,4R,5S)-3-(2-(difluoromethoxy)-4-fluoro-
3-methylphenyl)-4,5-dimethyl-N-(1-methyl-2-
oxo-1,2-dihydropyridin-4-yl)-5-
(trifluoromethyl)tetrahydrofuran-2-carboxamide

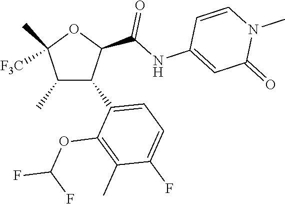 [SMILES: Cc1c(F)ccc([C@H]2[C@H](C(=O)Nc3ccn(C)c(=O)c3)O[C@@](C)(C(F)(F)F)[C@H]2C)c1OC(F)F]

(2R,3S,4S,5R)-3-(2-(difluoromethoxy)-4-fluoro-
3-methylphenyl)-4,5-dimethyl-N-(1-methyl-2-
oxo-1,2-dihydropyridin-4-yl)-5-
(trifluoromethyl)tetrahydrofuran-2-carboxamide

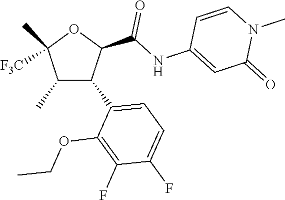 [SMILES: CCOc1c([C@H]2[C@H](C(=O)Nc3ccn(C)c(=O)c3)O[C@@](C)(C(F)(F)F)[C@H]2C)ccc(F)c1F]

(2R,3S,4S,5R)-3-(2-ethoxy-3,4-difluorophenyl)-
4,5-dimethyl-N-(1-methyl-2-oxo-1,2-
dihydropyridin-4-yl)-5-
(trifluoromethyl)tetrahydrofuran-2-carboxamide

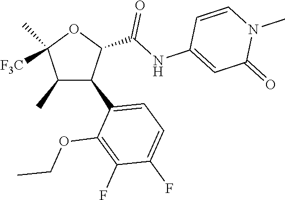 [SMILES: CCOc1c([C@@H]2[C@@H](C(=O)Nc3ccn(C)c(=O)c3)O[C@](C)(C(F)(F)F)[C@@H]2C)ccc(F)c1F]

(2S,3R,4R,5S)-3-(2-ethoxy-3,4-difluorophenyl)-
4,5-dimethyl-N-(1-methyl-2-oxo-1,2-
dihydropyridin-4-yl)-5-
(trifluoromethyl)tetrahydrofuran-2-carboxamide

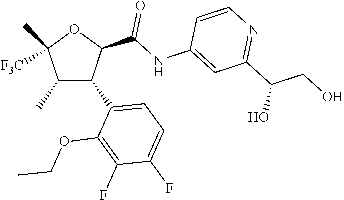 [SMILES: CCOc1c([C@H]2[C@H](C(=O)Nc3ccnc([C@@H](O)CO)c3)O[C@@](C)(C(F)(F)F)[C@H]2C)ccc(F)c1F]

(2R,3S,4S,5R)-N-(2-((R)-1,2-
dihydroxyethyl)pyridin-4-yl)-3-(2-ethoxy-3,4-
difluorophenyl)-4,5-dimethyl-5-
(trifluoromethyl)tetrahydrofuran-2-carboxamide

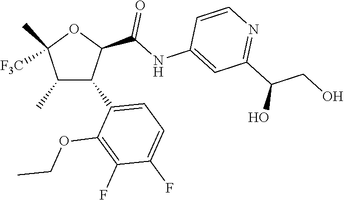 [SMILES: CCOc1c([C@H]2[C@H](C(=O)Nc3ccnc([C@H](O)CO)c3)O[C@@](C)(C(F)(F)F)[C@H]2C)ccc(F)c1F]

(2R,3S,4S,5R)-N-(2-((S)-1,2-
dihydroxyethyl)pyridin-4-yl)-3-(2-ethoxy-3,4-
difluorophenyl)-4,5-dimethyl-5-
(trifluoromethyl)tetrahydrofuran-2-carboxamide

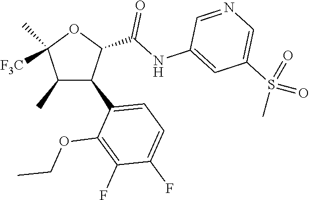 [SMILES: CCOc1c([C@@H]2[C@@H](C(=O)Nc3cncc(S(C)(=O)=O)c3)O[C@](C)(C(F)(F)F)[C@@H]2C)ccc(F)c1F]

(2S,3R,4R,5S)-3-(2-ethoxy-3,4-difluorophenyl)-
4,5-dimethyl-N-(5-(methylsulfonyl)pyridin-3-yl)-
5-(trifluoromethyl)tetrahydrofuran-2-
carboxamide

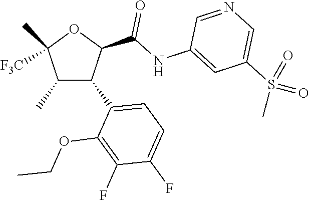 [SMILES: CCOc1c([C@H]2[C@H](C(=O)Nc3cncc(S(C)(=O)=O)c3)O[C@@](C)(C(F)(F)F)[C@H]2C)ccc(F)c1F]

(2R,3S,4S,5R)-3-(2-ethoxy-3,4-difluorophenyl)-
4,5-dimethyl-N-(5-(methylsulfonyl)pyridin-3-yl)-
5-(trifluoromethyl)tetrahydrofuran-2-
carboxamide

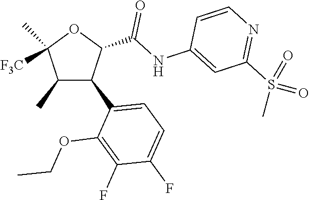 [SMILES: CCOc1c([C@@H]2[C@@H](C(=O)Nc3ccnc(S(C)(=O)=O)c3)O[C@](C)(C(F)(F)F)[C@@H]2C)ccc(F)c1F]

(2S,3R,4R,5S)-3-(2-ethoxy-3,4-difluorophenyl)-
4,5-dimethyl-N-(2-(methylsulfonyl)pyridin-4-yl)-
5-(trifluoromethyl)tetrahydrofuran-2-
carboxamide

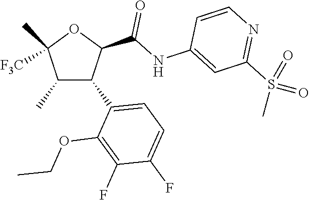 [SMILES: CCOc1c([C@H]2[C@H](C(=O)Nc3ccnc(S(C)(=O)=O)c3)O[C@@](C)(C(F)(F)F)[C@H]2C)ccc(F)c1F]

(2R,3S,4S,5R)-3-(2-ethoxy-3,4-difluorophenyl)-
4,5-dimethyl-N-(2-(methylsulfonyl)pyridin-4-yl)-
5-(trifluoromethyl)tetrahydrofuran-2-
carboxamide

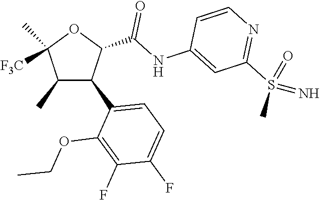 [SMILES: CCOc1c([C@@H]2[C@@H](C(=O)Nc3ccnc([S@@](C)(=N)=O)c3)O[C@](C)(C(F)(F)F)[C@@H]2C)ccc(F)c1F]

(2S,3R,4R,5S)-3-(2-ethoxy-3,4-difluorophenyl)-
4,5-dimethyl-N-(2-(S-
methylsulfonimidoyl)pyridin-4-yl)-5-
(trifluoromethyl)tetrahydrofuran-2-carboxamide

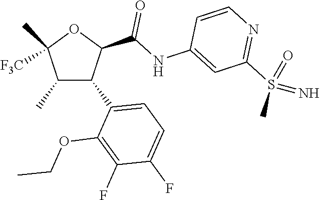 [SMILES: CCOc1c([C@H]2[C@H](C(=O)Nc3ccnc([S@@](C)(=N)=O)c3)O[C@@](C)(C(F)(F)F)[C@H]2C)ccc(F)c1F]

(2R,3S,4S,5R)-3-(2-ethoxy-3,4-difluorophenyl)-
4,5-dimethyl-N-(2-(S-
methylsulfonimidoyl)pyridin-4-yl)-5-
(trifluoromethyl)tetrahydrofuran-2-carboxamide

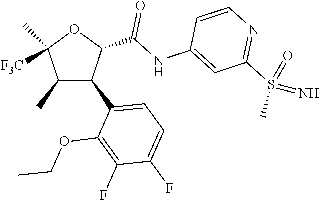 [SMILES: CCOc1c([C@@H]2[C@@H](C(=O)Nc3ccnc([S@](C)(=N)=O)c3)O[C@](C)(C(F)(F)F)[C@@H]2C)ccc(F)c1F]

(2S,3R,4R,5S)-3-(2-ethoxy-3,4-difluorophenyl)-
4,5-dimethyl-N-(2-(S-
methylsulfonimidoyl)pyridin-4-yl)-5-
(trifluoromethyl)tetrahydrofuran-2-carboxamide

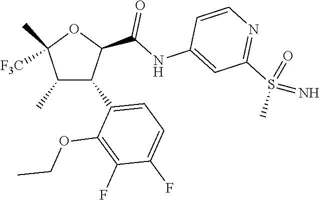 [SMILES: CCOc1c([C@H]2[C@H](C(=O)Nc3ccnc([S@](C)(=N)=O)c3)O[C@@](C)(C(F)(F)F)[C@H]2C)ccc(F)c1F]

(2R,3S,4S,5R)-3-(2-ethoxy-3,4-difluorophenyl)-
4,5-dimethyl-N-(2-(S-
methylsulfonimidoyl)pyridin-4-yl)-5-
(trifluoromethyl)tetrahydrofuran-2-carboxamide

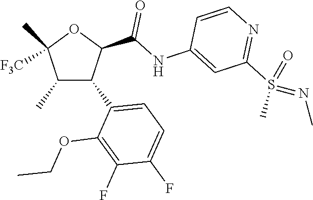 [SMILES: CCOc1c([C@H]2[C@H](C(=O)Nc3ccnc([S@@](C)(=O)=NC)c3)O[C@@](C)(C(F)(F)F)[C@H]2C)ccc(F)c1F]

(2R,3S,4S,5R)-N-(2-(N,S-
dimethylsulfonimidoyl)pyridin-4-yl)-3-(2-ethoxy-
3,4-difluorophenyl)-4,5-dimethyl-5-
(trifluoromethyl)tetrahydrofuran-2-carboxamide

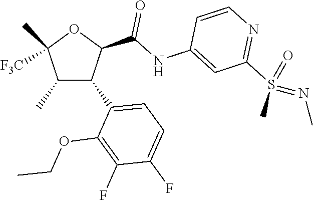 [SMILES: CCOc1c([C@H]2[C@H](C(=O)Nc3ccnc([S@](C)(=O)=NC)c3)O[C@@](C)(C(F)(F)F)[C@H]2C)ccc(F)c1F]

(2R,3S,4S,5R)-N-(2-(N,S-
dimethylsulfonimidoyl)pyridin-4-yl)-3-(2-ethoxy-
3,4-difluorophenyl)-4,5-dimethyl-5-
(trifluoromethyl)tetrahydrofuran-2-carboxamide

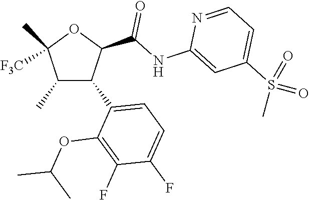 [SMILES: CC(C)Oc1c([C@H]2[C@H](C(=O)Nc3cc(S(C)(=O)=O)ccn3)O[C@@](C)(C(F)(F)F)[C@H]2C)ccc(F)c1F]

(2R,3S,4S,5R)-3-(3,4-difluoro-2-
isopropoxyphenyl)-4,5-dimethyl-N-(4-
(methylsulfonyl)pyridin-2-yl)-5-
(trifluoromethyl)tetrahydrofuran-2-carboxamide

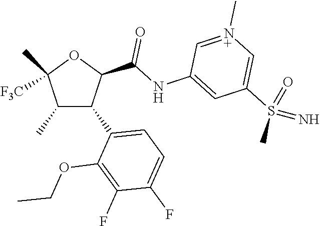 [SMILES: CCOc1c([C@H]2[C@H](C(=O)Nc3cc([S@@](C)(=N)=O)c[n+](C)c3)O[C@@](C)(C(F)(F)F)[C@H]2C)ccc(F)c1F]

3-((2R,3S,4S,5R)-3-(2-ethoxy-3,4-difluorophenyl)-
4,5-dimethyl-5-(trifluoromethyl)tetrahydrofuran-2-
carboxamido)-1-methyl-5-(S-
methylsulfonimidoyl)pyridin-1-ium

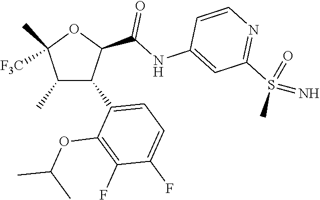 [SMILES: CC(C)Oc1c([C@H]2[C@H](C(=O)Nc3ccnc([S@@](C)(=N)=O)c3)O[C@@](C)(C(F)(F)F)[C@H]2C)ccc(F)c1F]

(2R,3S,4S,5R)-3-(3,4-difluoro-2-
isopropoxyphenyl)-4,5-dimethyl-N-(2-(S-
methylsulfonimidoyl)pyridin-4-yl)-5-
(trifluoromethyl)tetrahydrofuran-2-carboxamide

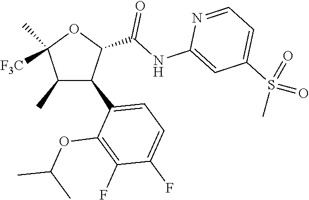 [SMILES: CC(C)Oc1c([C@@H]2[C@@H](C(=O)Nc3cc(S(C)(=O)=O)ccn3)O[C@](C)(C(F)(F)F)[C@@H]2C)ccc(F)c1F]

(2S,3R,4R,5S)-3-(3,4-difluoro-2-
isopropoxyphenyl)-4,5-dimethyl-N-(4-
(methylsulfonyl)pyridin-2-yl)-5-
(trifluoromethyl)tetrahydrofuran-2-carboxamide

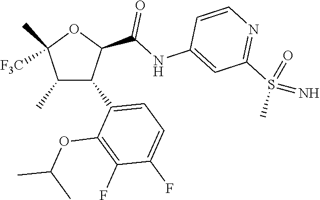 [SMILES: CC(C)Oc1c([C@H]2[C@H](C(=O)Nc3ccnc([S@](C)(=N)=O)c3)O[C@@](C)(C(F)(F)F)[C@H]2C)ccc(F)c1F]

(2R,3S,4S,5R)-3-(3,4-difluoro-2-
isopropoxyphenyl)-4,5-dimethyl-N-(2-(S-
methylsulfonimidoyl)pyridin-4-yl)-5-
(trifluoromethyl)tetrahydrofuran-2-carboxamide

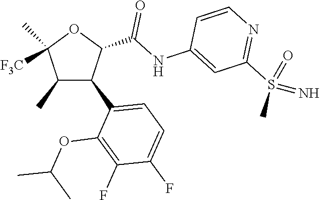 [SMILES: CC(C)Oc1c([C@@H]2[C@@H](C(=O)Nc3ccnc([S@@](C)(=N)=O)c3)O[C@](C)(C(F)(F)F)[C@@H]2C)ccc(F)c1F]

(2S,3R,4R,5S)-3-(3,4-difluoro-2-
isopropoxyphenyl)-4,5-dimethyl-N-(2-(S-
methylsulfonimidoyl)pyridin-4-yl)-5-
(trifluoromethyl)tetrahydrofuran-2-carboxamide

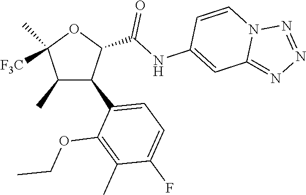 [SMILES: CCOc1c([C@@H]2[C@@H](C(=O)Nc3ccn4nnnc4c3)O[C@](C)(C(F)(F)F)[C@@H]2C)ccc(F)c1C]

(2S,3R,4R,5S)-3-(2-ethoxy-4-fluoro-3-
methylphenyl)-4,5-dimethyl-N-(tetrazolo[1,5-
a]pyridin-7-yl)-5-
(trifluoromethyl)tetrahydrofuran-2-carboxamide

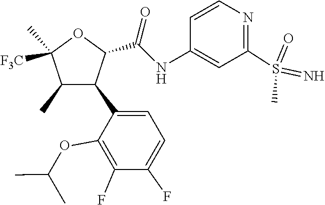 [SMILES: CC(C)Oc1c([C@@H]2[C@@H](C(=O)Nc3ccnc([S@](C)(=N)=O)c3)O[C@](C)(C(F)(F)F)[C@@H]2C)ccc(F)c1F]

(2S,3R,4R,5S)-3-(3,4-difluoro-2-
isopropoxyphenyl)-4,5-dimethyl-N-(2-(S-
methylsulfonimidoyl)pyridin-4-yl)-5-
(trifluoromethyl)tetrahydrofuran-2-carboxamide

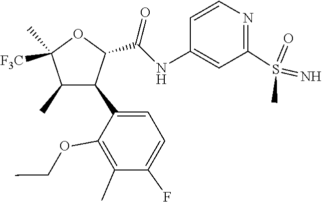 [SMILES: CCOc1c([C@@H]2[C@@H](C(=O)Nc3ccnc([S@@](C)(=N)=O)c3)O[C@](C)(C(F)(F)F)[C@@H]2C)ccc(F)c1C]

(2S,3R,4R,5S)-3-(2-ethoxy-4-fluoro-3-
methylphenyl)-4,5-dimethyl-N-(2-(S-
methylsulfonimidoyl)pyridin-4-yl)-5-
(trifluoromethyl)tetrahydrofuran-2-carboxamide

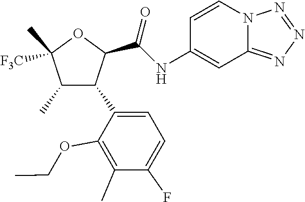 [SMILES: CCOc1c([C@H]2[C@H](C(=O)Nc3ccn4nnnc4c3)O[C@@](C)(C(F)(F)F)[C@H]2C)ccc(F)c1C]

(2R,3S,4S,5R)-3-(2-ethoxy-4-fluoro-3-
methylphenyl)-4,5-dimethyl-N-(tetrazolo[1,5-
a]pyridin-7-yl)-5-
(trifluoromethyl)tetrahydrofuran-2-carboxamide

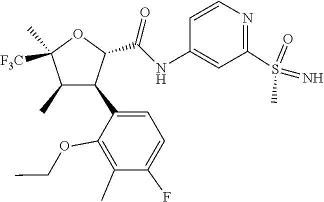 [SMILES: CCOc1c([C@@H]2[C@@H](C(=O)Nc3ccnc([S@](C)(=N)=O)c3)O[C@](C)(C(F)(F)F)[C@@H]2C)ccc(F)c1C]

(2S,3R,4R,5S)-3-(2-ethoxy-4-fluoro-3-
methylphenyl)-4,5-dimethyl-N-(2-(S-
methylsulfonimidoyl)pyridin-4-yl)-5-
(trifluoromethyl)tetrahydrofuran-2-carboxamide

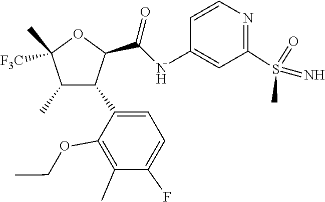 [SMILES: CCOc1c([C@H]2[C@H](C(=O)Nc3ccnc([S@@](C)(=N)=O)c3)O[C@@](C)(C(F)(F)F)[C@H]2C)ccc(F)c1C]

(2R,3S,4S,5R)-3-(2-ethoxy-4-fluoro-3-
methylphenyl)-4,5-dimethyl-N-(2-(S-
methylsulfonimidoyl)pyridin-4-yl)-5-
(trifluoromethyl)tetrahydrofuran-2-carboxamide

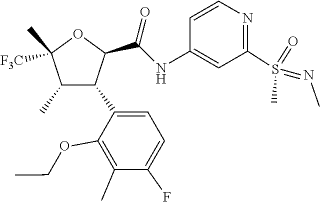 [SMILES: CCOc1c([C@H]2[C@H](C(=O)Nc3ccnc([S@@](C)(=O)=NC)c3)O[C@@](C)(C(F)(F)F)[C@H]2C)ccc(F)c1C]

(2R,3S,4S,5R)-N-(2-(N,S-
dimethylsulfonimidoyl)pyridin-4-yl)-3-(2-ethoxy-
4-fluoro-3-methylphenyl)-4,5-dimethyl-5-
(trifluoromethyl)tetrahydrofuran-2-carboxamide

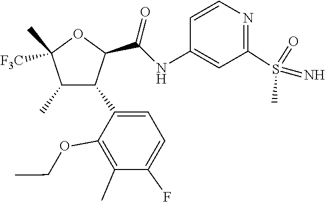 [SMILES: CCOc1c([C@H]2[C@H](C(=O)Nc3ccnc([S@](C)(=N)=O)c3)O[C@@](C)(C(F)(F)F)[C@H]2C)ccc(F)c1C]

(2R,3S,4S,5R)-3-(2-ethoxy-4-fluoro-3-
methylphenyl)-4,5-dimethyl-N-(2-(S-
methylsulfonimidoyl)pyridin-4-yl)-5-
(trifluoromethyl)tetrahydrofuran-2-carboxamide

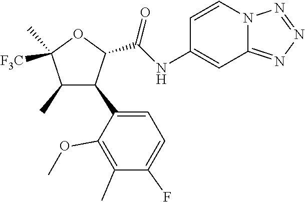 [SMILES: COc1c([C@@H]2[C@@H](C(=O)Nc3ccn4nnnc4c3)O[C@](C)(C(F)(F)F)[C@@H]2C)ccc(F)c1C]

(2S,3R,4R,5S)-3-(4-fluoro-2-methoxy-3-
methylphenyl)-4,5-dimethyl-N-(tetrazolo[1,5-
a]pyridin-7-yl)-5-
(trifluoromethyl)tetrahydrofuran-2-carboxamide

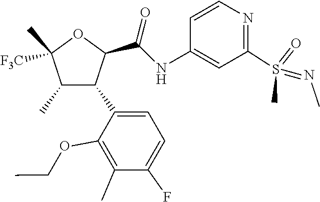 [SMILES: CCOc1c([C@H]2[C@H](C(=O)Nc3ccnc([S@](C)(=O)=NC)c3)O[C@@](C)(C(F)(F)F)[C@H]2C)ccc(F)c1C]

(2R,3S,4S,5R)-N-(2-(N,S-
dimethylsulfonimidoyl)pyridin-4-yl)-3-(2-ethoxy-
4-fluoro-3-methylphenyl)-4,5-dimethyl-5-
(trifluoromethyl)tetrahydrofuran-2-carboxamide

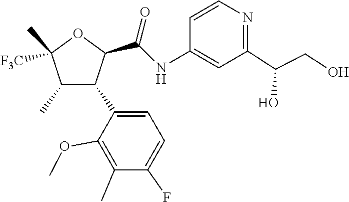 [SMILES: COc1c([C@H]2[C@H](C(=O)Nc3ccnc([C@@H](O)CO)c3)O[C@@](C)(C(F)(F)F)[C@H]2C)ccc(F)c1C]

(2R,3S,4S,5R)-N-(2-((R)-1,2-
dihydroxyethyl)pyridin-4-yl)-3-(4-fluoro-2-
methoxy-3-methylphenyl)-4,5-dimethyl-5-
(trifluoromethyl)tetrahydrofuran-2-carboxamide

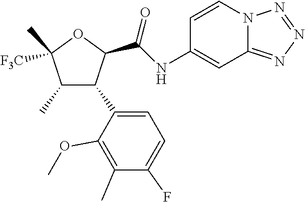 [SMILES: COc1c([C@H]2[C@H](C(=O)Nc3ccn4nnnc4c3)O[C@@](C)(C(F)(F)F)[C@H]2C)ccc(F)c1C]

(2R,3S,4S,5R)-3-(4-fluoro-2-methoxy-3-
methylphenyl)-4,5-dimethyl-N-(tetrazolo[1,5-
a]pyridin-7-yl)-5-
(trifluoromethyl)tetrahydrofuran-2-carboxamide

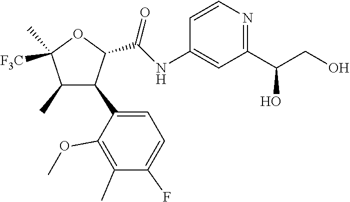 [SMILES: COc1c([C@@H]2[C@@H](C(=O)Nc3ccnc([C@H](O)CO)c3)O[C@](C)(C(F)(F)F)[C@@H]2C)ccc(F)c1C]

(2S,3R,4R,5S)-N-(2-((S)-1,2-
dihydroxyethyl)pyridin-4-yl)-3-(4-fluoro-2-
methoxy-3-methylphenyl)-4,5-dimethyl-5-
(trifluoromethyl)tetrahydrofuran-2-carboxamide

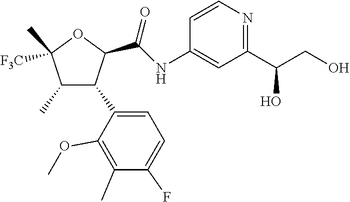 [SMILES: COc1c([C@H]2[C@H](C(=O)Nc3ccnc([C@H](O)CO)c3)O[C@@](C)(C(F)(F)F)[C@H]2C)ccc(F)c1C]

(2R,3S,4S,5R)-N-(2-((S)-1,2-
dihydroxyethyl)pyridin-4-yl)-3-(4-fluoro-2-
methoxy-3-methylphenyl)-4,5-dimethyl-5-
(trifluoromethyl)tetrahydrofuran-2-carboxamide

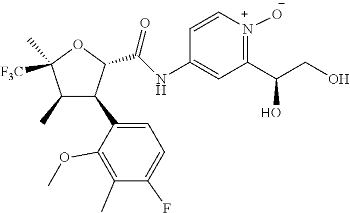 [SMILES: COc1c([C@@H]2[C@@H](C(=O)Nc3cc[n+]([O-])c([C@H](O)CO)c3)O[C@](C)(C(F)(F)F)[C@@H]2C)ccc(F)c1C]

2-((S)-1,2-dihydroxyethyl)-4-((2S,3R,4R,5S)-3-
(4-fluoro-2-methoxy-3-methylphenyl)-4,5-
dimethyl-5-(trifluoromethyl)tetrahydrofuran-2-
carboxamido)pyridine 1-oxide

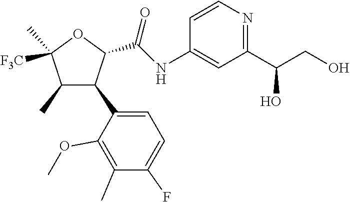 [SMILES: COc1c([C@@H]2[C@@H](C(=O)Nc3ccnc([C@H](O)CO)c3)O[C@](C)(C(F)(F)F)[C@@H]2C)ccc(F)c1C]

(2S,3R,4R,5S)-N-(2-((S)-1,2-
dihydroxyethyl)pyridin-4-yl)-3-(4-fluoro-2-
methoxy-3-methylphenyl)-4,5-dimethyl-5-
(trifluoromethyl)tetrahydrofuran-2-carboxamide

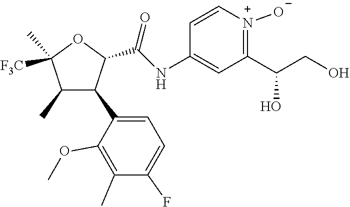 [SMILES: COc1c([C@@H]2[C@@H](C(=O)Nc3cc[n+]([O-])c([C@@H](O)CO)c3)O[C@](C)(C(F)(F)F)[C@@H]2C)ccc(F)c1C]

2-((R)-1,2-dihydroxyethyl)-4-((2S,3R,4R,5S)-3-
(4-fluoro-2-methoxy-3-methylphenyl)-4,5-
dimethyl-5-(trifluoromethyl)tetrahydrofuran-2-
carboxamido)pyridine 1-oxide

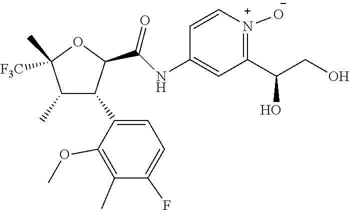 [SMILES: COc1c([C@H]2[C@H](C(=O)Nc3cc[n+]([O-])c([C@H](O)CO)c3)O[C@@](C)(C(F)(F)F)[C@H]2C)ccc(F)c1C]

2-((S)-1,2-dihydroxyethyl)-4-((2R,3S,4S,5R)-3-
(4-fluoro-2-methoxy-3-methylphenyl)-4,5-
dimethyl-5-(trifluoromethyl)tetrahydrofuran-2-
carboxamido)pyridine 1-oxide

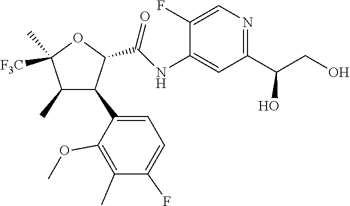 [SMILES: COc1c([C@@H]2[C@@H](C(=O)Nc3cc([C@H](O)CO)ncc3F)O[C@](C)(C(F)(F)F)[C@@H]2C)ccc(F)c1C]

(2S,3R,4R,5S)-N-(2-((S)-1,2-dihydroxyethyl)-5-
fluoropyridin-4-yl)-3-(4-fluoro-2-methoxy-3-
methylphenyl)-4,5-dimethyl-5-
(trifluoromethyl)tetrahydrofuran-2-carboxamide

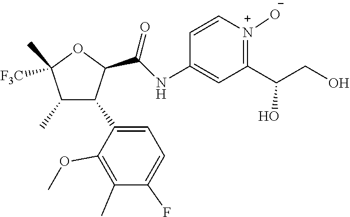 [SMILES: COc1c([C@H]2[C@H](C(=O)Nc3cc[n+]([O-])c([C@@H](O)CO)c3)O[C@@](C)(C(F)(F)F)[C@H]2C)ccc(F)c1C]

2-((R)-1,2-dihydroxyethyl)-4-((2R,3S,4S,5R)-3-
(4-fluoro-2-methoxy-3-methylphenyl)-4,5-
dimethyl-5-(trifluoromethyl)tetrahydrofuran-2-
carboxamido)pyridine 1-oxide

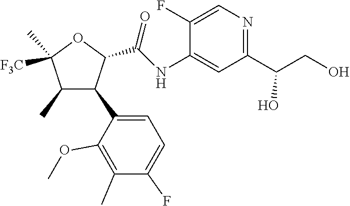 [SMILES: COc1c([C@@H]2[C@@H](C(=O)Nc3cc([C@@H](O)CO)ncc3F)O[C@](C)(C(F)(F)F)[C@@H]2C)ccc(F)c1C]

(2S,3R,4R,5S)-N-(2-((R)-1,2-dihydroxyethyl)-5-
fluoropyridin-4-yl)-3-(4-fluoro-2-methoxy-3-
methylphenyl)-4,5-dimethyl-5-
(trifluoromethyl)tetrahydrofuran-2-carboxamide

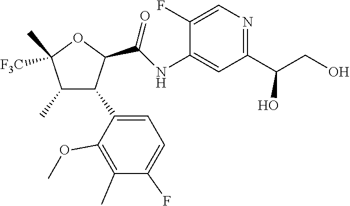 [SMILES: COc1c([C@H]2[C@H](C(=O)Nc3cc([C@H](O)CO)ncc3F)O[C@@](C)(C(F)(F)F)[C@H]2C)ccc(F)c1C]

(2R,3S,4S,5R)-N-(2-((S)-1,2-dihydroxyethyl)-5-
fluoropyridin-4-yl)-3-(4-fluoro-2-methoxy-3-
methylphenyl)-4,5-dimethyl-5-
(trifluoromethyl)tetrahydrofuran-2-carboxamide

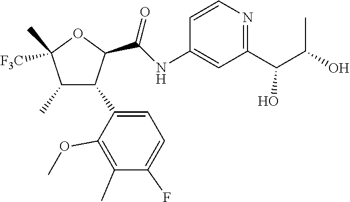 [SMILES: COc1c([C@H]2[C@H](C(=O)Nc3ccnc([C@@H](O)[C@H](C)O)c3)O[C@@](C)(C(F)(F)F)[C@H]2C)ccc(F)c1C]

(2R,3S,4S,5R)-N-(2-((1R,2S)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

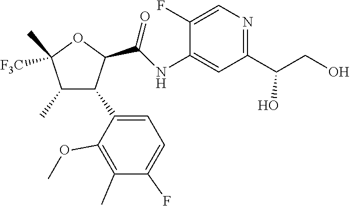 [SMILES: COc1c([C@H]2[C@H](C(=O)Nc3cc([C@@H](O)CO)ncc3F)O[C@@](C)(C(F)(F)F)[C@H]2C)ccc(F)c1C]

(2R,3S,4S,5R)-N-(2-((R)-1,2-dihydroxyethyl)-5-
fluoropyridin-4-yl)-3-(4-fluoro-2-methoxy-3-
methylphenyl)-4,5-dimethyl-5-
(trifluoromethyl)tetrahydrofuran-2-carboxamide

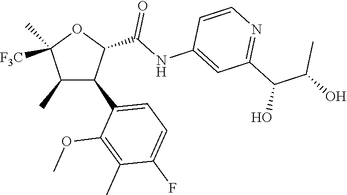 [SMILES: COc1c([C@@H]2[C@@H](C(=O)Nc3ccnc([C@@H](O)[C@H](C)O)c3)O[C@](C)(C(F)(F)F)[C@@H]2C)ccc(F)c1C]

(2S,3R,4R,5S)-N-(2-((1R,2S)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

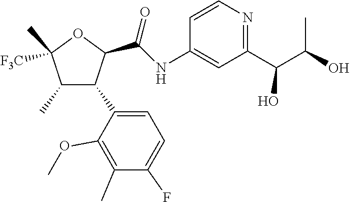 [SMILES: COc1c([C@H]2[C@H](C(=O)Nc3ccnc([C@H](O)[C@@H](C)O)c3)O[C@@](C)(C(F)(F)F)[C@H]2C)ccc(F)c1C]

(2R,3S,4S,5R)-N-(2-((1S,2R)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

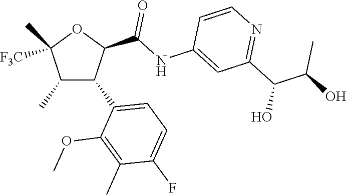 [SMILES: COc1c([C@H]2[C@H](C(=O)Nc3ccnc([C@@H](O)[C@@H](C)O)c3)O[C@@](C)(C(F)(F)F)[C@H]2C)ccc(F)c1C]

(2R,3S,4S,5R)-N-(2-((1R,2R)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

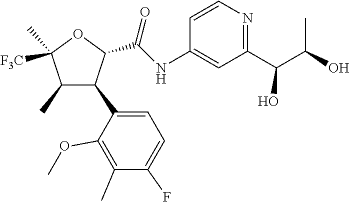 [SMILES: COc1c([C@@H]2[C@@H](C(=O)Nc3ccnc([C@H](O)[C@@H](C)O)c3)O[C@](C)(C(F)(F)F)[C@@H]2C)ccc(F)c1C]

(2S,3R,4R,5S)-N-(2-((1S,2R)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

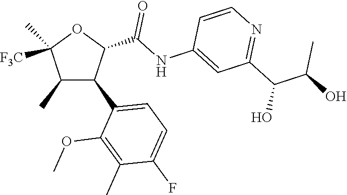 [SMILES: COc1c([C@@H]2[C@@H](C(=O)Nc3ccnc([C@@H](O)[C@@H](C)O)c3)O[C@](C)(C(F)(F)F)[C@@H]2C)ccc(F)c1C]

(2S,3R,4R,5S)-N-(2-((1R,2R)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

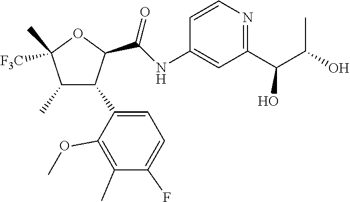 [SMILES: COc1c([C@H]2[C@H](C(=O)Nc3ccnc([C@H](O)[C@H](C)O)c3)O[C@@](C)(C(F)(F)F)[C@H]2C)ccc(F)c1C]

(2R,3S,4S,5R)-N-(2-((1S,2S)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

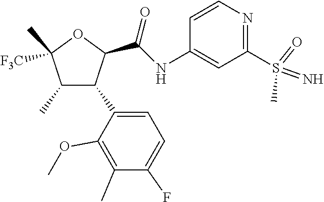 [SMILES: COc1c([C@H]2[C@H](C(=O)Nc3ccnc([S@](C)(=N)=O)c3)O[C@@](C)(C(F)(F)F)[C@H]2C)ccc(F)c1C]

(2R,3S,4S,5R)-3-(4-fluoro-2-methoxy-3-
methylphenyl)-4,5-dimethyl-N-(2-(S-
methylsulfonimidoyl)pyridin-4-yl)-5-
(trifluoromethyl)tetrahydrofuran-2-carboxamide

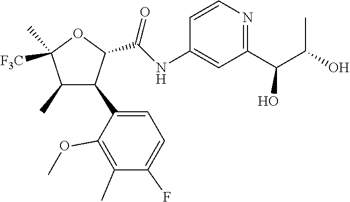 [SMILES: COc1c([C@@H]2[C@@H](C(=O)Nc3ccnc([C@H](O)[C@H](C)O)c3)O[C@](C)(C(F)(F)F)[C@@H]2C)ccc(F)c1C]

(2S,3R,4R,5S)-N-(2-((1S,2S)-1,2-
dihydroxypropyl)pyridin-4-yl)-3-(4-fluoro-2-
methoxy-3-methylphenyl)-4,5-dimethyl-5-
(trifluoromethyl)tetrahydrofuran-2-carboxamide

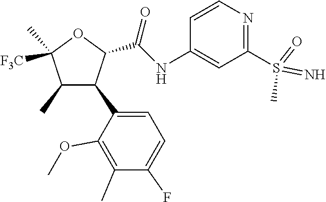 [SMILES: COc1c([C@@H]2[C@@H](C(=O)Nc3ccnc([S@](C)(=N)=O)c3)O[C@](C)(C(F)(F)F)[C@@H]2C)ccc(F)c1C]

(2S,3R,4R,5S)-3-(4-fluoro-2-methoxy-3-
methylphenyl)-4,5-dimethyl-N-(2-(S-
methylsulfonimidoyl)pyridin-4-yl)-5-
(trifluoromethyl)tetrahydrofuran-2-carboxamide

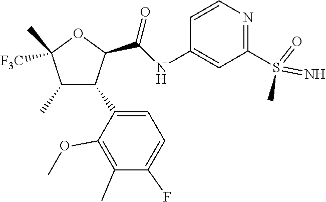 [SMILES: COc1c([C@H]2[C@H](C(=O)Nc3ccnc([S@@](C)(=N)=O)c3)O[C@@](C)(C(F)(F)F)[C@H]2C)ccc(F)c1C]

(2R,3S,4S,5R)-3-(4-fluoro-2-methoxy-3-
methylphenyl)-4,5-dimethyl-N-(2-(S-
methylsulfonimidoyl)pyridin-4-yl)-5-
(trifluoromethyl)tetrahydrofuran-2-carboxamide

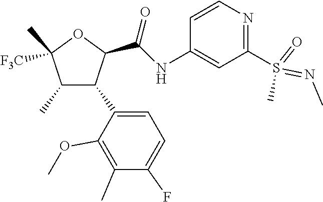 [SMILES: CN=[S@](C)(=O)c1cc(NC(=O)[C@@H]2O[C@@](C)(C(F)(F)F)[C@@H](C)[C@H]2c2ccc(F)c(C)c2OC)ccn1]

(2R,3S,4S,5R)-N-(2-(N,S-
dimethylsulfonimidoyl)pyridin-4-yl)-3-(4-fluoro-
2-methoxy-3-methylphenyl)-4,5-dimethyl-5-
(trifluoromethyl)tetrahydrofuran-2-carboxamide

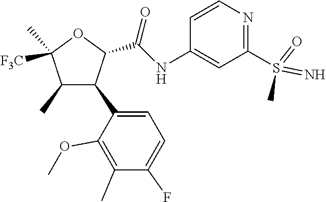 [SMILES: COc1c([C@@H]2[C@@H](C(=O)Nc3ccnc([S@@](C)(=N)=O)c3)O[C@](C)(C(F)(F)F)[C@@H]2C)ccc(F)c1C]

(2S,3R,4R,5S)-3-(4-fluoro-2-methoxy-3-
methylphenyl)-4,5-dimethyl-N-(2-(S-
methylsulfonimidoyl)pyridin-4-yl)-5-
(trifluoromethyl)tetrahydrofuran-2-carboxamide

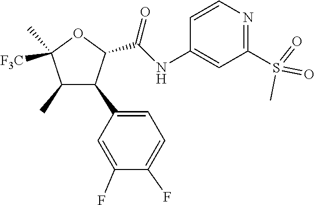 [SMILES: C[C@@H]1[C@H](c2ccc(F)c(F)c2)[C@@H](C(=O)Nc2ccnc(S(C)(=O)=O)c2)O[C@]1(C)C(F)(F)F]

(2S,3R,4R,5S)-3-(3,4-difluorophenyl)-4,5-
dimethyl-N-(2-(methylsulfonyl)pyridin-4-yl)-5-
(trifluoromethyl)tetrahydrofuran-2-carboxamide

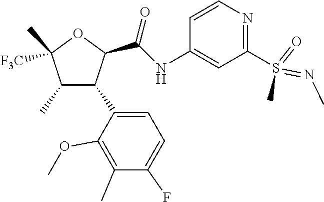 [SMILES: CN=[S@@](C)(=O)c1cc(NC(=O)[C@@H]2O[C@@](C)(C(F)(F)F)[C@@H](C)[C@H]2c2ccc(F)c(C)c2OC)ccn1]

(2R,3S,4S,5R)-N-(2-(N,S-
dimethylsulfonimidoyl)pyridin-4-yl)-3-(4-fluoro-
2-methoxy-3-methylphenyl)-4,5-dimethyl-5-
(trifluoromethyl)tetrahydrofuran-2-carboxamide

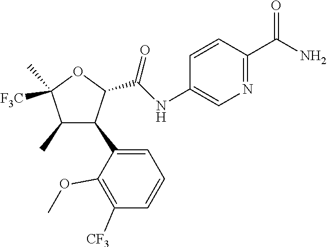 [SMILES: COc1c([C@@H]2[C@@H](C(=O)Nc3ccc(C(N)=O)nc3)O[C@](C)(C(F)(F)F)[C@@H]2C)cccc1C(F)(F)F]

5-((2S,3R,4R,5S)-3-(2-methoxy-3-
(trifluoromethyl)phenyl)-4,5-dimethyl-5-
(trifluoromethyl)tetrahydrofuran-2-
carboxamido)picolinamide

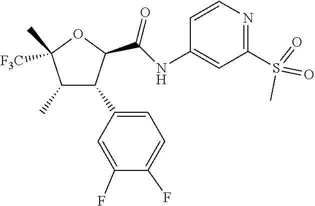 [SMILES: C[C@H]1[C@@H](c2ccc(F)c(F)c2)[C@H](C(=O)Nc2ccnc(S(C)(=O)=O)c2)O[C@@]1(C)C(F)(F)F]

(2R,3S,4S,5R)-3-(3,4-difluorophenyl)-4,5-
dimethyl-N-(2-(methylsulfonyl)pyridin-4-yl)-5-
(trifluoromethyl)tetrahydrofuran-2-carboxamide

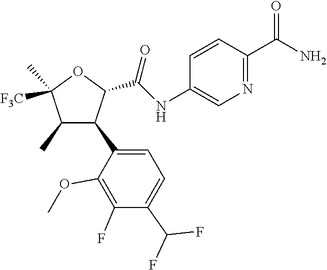 [SMILES: COc1c([C@@H]2[C@@H](C(=O)Nc3ccc(C(N)=O)nc3)O[C@](C)(C(F)(F)F)[C@@H]2C)ccc(C(F)F)c1F]

5-((2S,3R,4R,5S)-3-(4-(difluoromethyl)-3-fluoro-
2-methoxyphenyl)-4,5-dimethyl-5-
(trifluoromethyl)tetrahydrofuran-2-
carboxamido)picolinamide

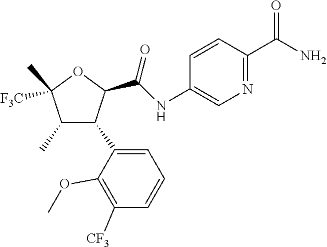 [SMILES: COc1c([C@H]2[C@H](C(=O)Nc3ccc(C(N)=O)nc3)O[C@@](C)(C(F)(F)F)[C@H]2C)cccc1C(F)(F)F]

5-((2R,3S,4S,5R)-3-(2-methoxy-3-
(trifluoromethyl)phenyl)-4,5-dimethyl-5-
(trifluoromethyl)tetrahydrofuran-2-
carboxamido)picolinamide

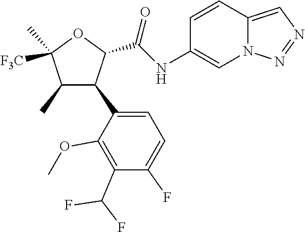 [SMILES: COc1c([C@@H]2[C@@H](C(=O)Nc3ccc4cnnn4c3)O[C@](C)(C(F)(F)F)[C@@H]2C)ccc(F)c1C(F)F]

(2S,3R,4R,5S)-N-([1,2,3]triazolo[1,5-a]pyridin-6-
yl)-3-(3-(difluoromethyl)-4-fluoro-2-
methoxyphenyl)-4,5-dimethyl-5-
(trifluoromethyl)tetrahydrofuran-2-carboxamide

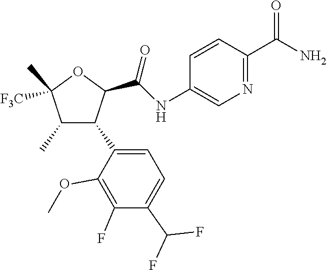 [SMILES: COc1c([C@H]2[C@H](C(=O)Nc3ccc(C(N)=O)nc3)O[C@@](C)(C(F)(F)F)[C@H]2C)ccc(C(F)F)c1F]

5-((2R,3S,4S,5R)-3-(4-(difluoromethyl)-3-fluoro-
2-methoxyphenyl)-4,5-dimethyl-5-
(trifluoromethyl)tetrahydrofuran-2-
carboxamido)picolinamide

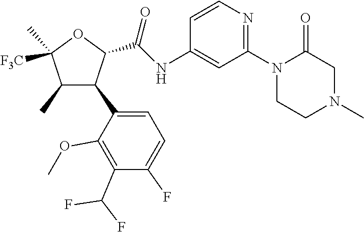 [SMILES: COc1c([C@@H]2[C@@H](C(=O)Nc3ccnc(N4CCN(C)CC4=O)c3)O[C@](C)(C(F)(F)F)[C@@H]2C)ccc(F)c1C(F)F]

(2S,3R,4R,5S)-3-(3-(difluoromethyl)-4-fluoro-2-
methoxyphenyl)-4,5-dimethyl-N-(2-(4-methyl-2-
oxopiperazin-1-yl)pyridin-4-yl)-5-
(trifluoromethyl)tetrahydrofuran-2-carboxamide

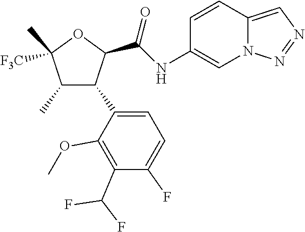 [SMILES: COc1c([C@H]2[C@H](C(=O)Nc3ccc4cnnn4c3)O[C@@](C)(C(F)(F)F)[C@H]2C)ccc(F)c1C(F)F]

(2R,3S,4S,5R)-N-([1,2,3]triazolo[1,5-a]pyridin-
6-yl)-3-(3-(difluoromethyl)-4-fluoro-2-
methoxyphenyl)-4,5-dimethyl-5-
(trifluoromethyl)tetrahydrofuran-2-carboxamide

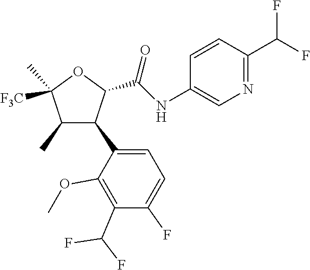 [SMILES: COc1c([C@@H]2[C@@H](C(=O)Nc3ccc(C(F)F)nc3)O[C@](C)(C(F)(F)F)[C@@H]2C)ccc(F)c1C(F)F]

(2S,3R,4R,5S)-3-(3-(difluoromethyl)-4-fluoro-2-
methoxyphenyl)-N-(6-(difluoromethyl)pyridin-3-
yl)-4,5-dimethyl-5-
(trifluoromethyl)tetrahydrofuran-2-carboxamide

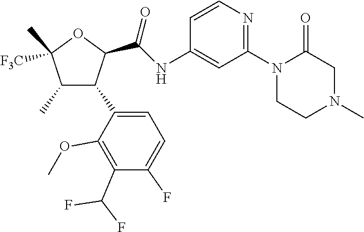 [SMILES: COc1c([C@H]2[C@H](C(=O)Nc3ccnc(N4CCN(C)CC4=O)c3)O[C@@](C)(C(F)(F)F)[C@H]2C)ccc(F)c1C(F)F]

(2R,3S,4S,5R)-3-(3-(difluoromethyl)-4-fluoro-2-
methoxyphenyl)-4,5-dimethyl-N-(2-(4-methyl-2-
oxopiperazin-1-yl)pyridin-4-yl)-5-
(trifluoromethyl)tetrahydrofuran-2-carboxamide

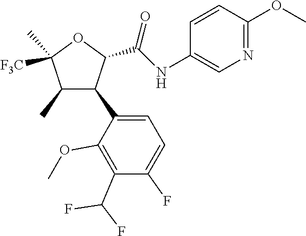 [SMILES: COc1ccc(NC(=O)[C@H]2O[C@](C)(C(F)(F)F)[C@H](C)[C@@H]2c2ccc(F)c(C(F)F)c2OC)cn1]

(2S,3R,4R,5S)-3-(3-(difluoromethyl)-4-fluoro-2-
methoxyphenyl)-N-(6-methoxypyridin-3-yl)-4,5-
dimethyl-5-(trifluoromethyl)tetrahydrofuran-2-
carboxamide

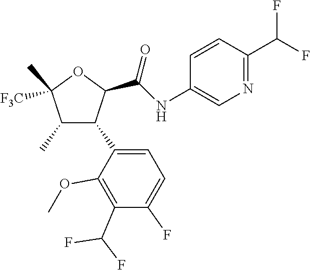 [SMILES: COc1c([C@H]2[C@H](C(=O)Nc3ccc(C(F)F)nc3)O[C@@](C)(C(F)(F)F)[C@H]2C)ccc(F)c1C(F)F]

(2R,3S,4S,5R)-3-(3-(difluoromethyl)-4-fluoro-2-
methoxyphenyl)-N-(6-(difluoromethyl)pyridin-3-
yl)-4,5-dimethyl-5-
(trifluoromethyl)tetrahydrofuran-2-carboxamide

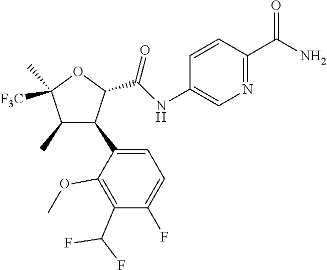 [SMILES: COc1c([C@@H]2[C@@H](C(=O)Nc3ccc(C(N)=O)nc3)O[C@](C)(C(F)(F)F)[C@@H]2C)ccc(F)c1C(F)F]

5-((2S,3R,4R,5S)-3-(3-(difluoromethyl)-4-fluoro-
2-methoxyphenyl)-4,5-dimethyl-5-
(trifluoromethyl)tetrahydrofuran-2-
carboxamido)picolinamide

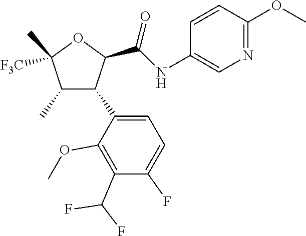 [SMILES: COc1ccc(NC(=O)[C@@H]2O[C@@](C)(C(F)(F)F)[C@@H](C)[C@H]2c2ccc(F)c(C(F)F)c2OC)cn1]

(2R,3S,4S,5R)-3-(3-(difluoromethyl)-4-fluoro-2-
methoxyphenyl)-N-(6-methoxypyridin-3-yl)-4,5-
dimethyl-5-(trifluoromethyl)tetrahydrofuran-2-
carboxamide

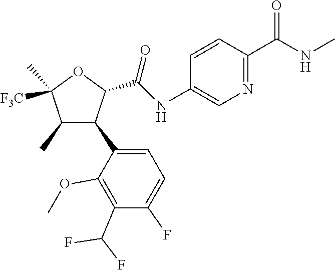 [SMILES: CNC(=O)c1ccc(NC(=O)[C@H]2O[C@](C)(C(F)(F)F)[C@H](C)[C@@H]2c2ccc(F)c(C(F)F)c2OC)cn1]

5-((2S,3R,4R,5S)-3-(3-(difluoromethyl)-4-fluoro-
2-methoxyphenyl)-4,5-dimethyl-5-
(trifluoromethyl)tetrahydrofuran-2-
carboxamido)-N-methylpicolinamide

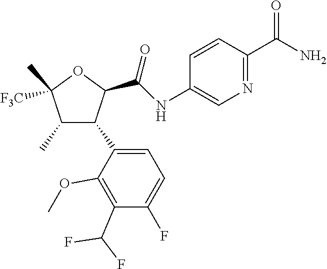 [SMILES: COc1c([C@H]2[C@H](C(=O)Nc3ccc(C(N)=O)nc3)O[C@@](C)(C(F)(F)F)[C@H]2C)ccc(F)c1C(F)F]

5-((2R,3S,4S,5R)-3-(3-(difluoromethyl)-4-fluoro-
2-methoxyphenyl)-4,5-dimethyl-5-
(trifluoromethyl)tetrahydrofuran-2-
carboxamido)picolinamide

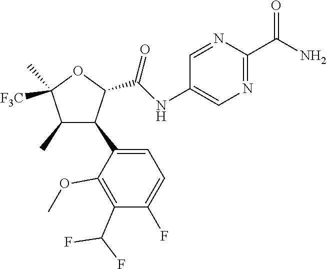 [SMILES: COc1c([C@@H]2[C@@H](C(=O)Nc3cnc(C(N)=O)nc3)O[C@](C)(C(F)(F)F)[C@@H]2C)ccc(F)c1C(F)F]

5-((2S,3R,4R,5S)-3-(3-(difluoromethyl)-4-fluoro-
2-methoxyphenyl)-4,5-dimethyl-5-
(trifluoromethyl)tetrahydrofuran-2-
carboxamido)pyrimidine-2-carboxamide

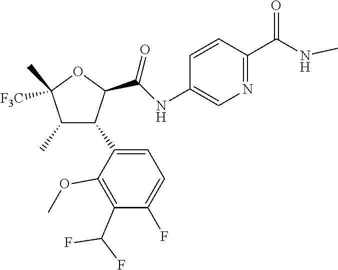 [SMILES: CNC(=O)c1ccc(NC(=O)[C@@H]2O[C@@](C)(C(F)(F)F)[C@@H](C)[C@H]2c2ccc(F)c(C(F)F)c2OC)cn1]

5-((2R,3S,4S,5R)-3-(3-(difluoromethyl)-4-fluoro-
2-methoxyphenyl)-4,5-dimethyl-5-
(trifluoromethyl)tetrahydrofuran-2-
carboxamido)-N-methylpicolinamide

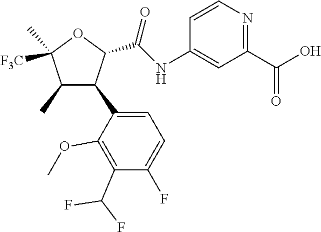 [SMILES: COc1c([C@@H]2[C@@H](C(=O)Nc3ccnc(C(=O)O)c3)O[C@](C)(C(F)(F)F)[C@@H]2C)ccc(F)c1C(F)F]

4-((2S,3R,4R,5S)-3-(3-(difluoromethyl)-4-fluoro-
2-methoxyphenyl)-4,5-dimethyl-5-
(trifluoromethyl)tetrahydrofuran-2-
carboxamido)picolinic acid

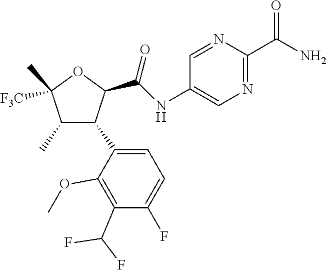 [SMILES: COc1c([C@H]2[C@H](C(=O)Nc3cnc(C(N)=O)nc3)O[C@@](C)(C(F)(F)F)[C@H]2C)ccc(F)c1C(F)F]

5-((2R,3S,4S,5R)-3-(3-(difluoromethyl)-4-fluoro-
2-methoxyphenyl)-4,5-dimethyl-5-
(trifluoromethyl)tetrahydrofuran-2-
carboxamido)pyrimidine-2-carboxamide

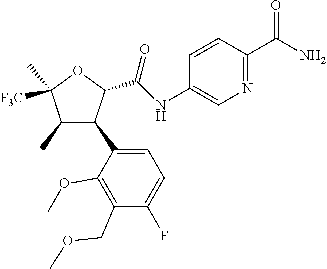 [SMILES: COCc1c(F)ccc([C@@H]2[C@@H](C(=O)Nc3ccc(C(N)=O)nc3)O[C@](C)(C(F)(F)F)[C@@H]2C)c1OC]

5-((2S,3R,4R,5S)-3-(4-fluoro-2-methoxy-3-
(methoxymethyl)phenyl)-4,5-dimethyl-5-
(trifluoromethyl)tetrahydrofuran-2-
carboxamido)picolinamide

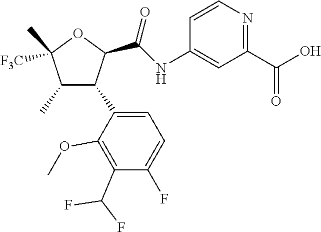 [SMILES: COc1c([C@H]2[C@H](C(=O)Nc3ccnc(C(=O)O)c3)O[C@@](C)(C(F)(F)F)[C@H]2C)ccc(F)c1C(F)F]

4-((2R,3S,4S,5R)-3-(3-(difluoromethyl)-4-fluoro-
2-methoxyphenyl)-4,5-dimethyl-5-
(trifluoromethyl)tetrahydrofuran-2-
carboxamido)picolinic acid

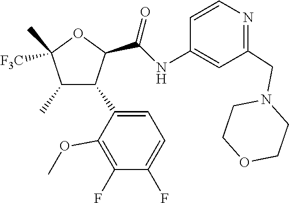 [SMILES: COc1c([C@H]2[C@H](C(=O)Nc3ccnc(CN4CCOCC4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(morpholinomethyl)pyridin-4-yl)-
5-(trifluoromethyl)tetrahydrofuran-2-carboxamide

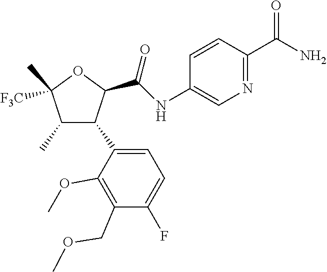 [SMILES: COCc1c(F)ccc([C@H]2[C@H](C(=O)Nc3ccc(C(N)=O)nc3)O[C@@](C)(C(F)(F)F)[C@H]2C)c1OC]

5-((2R,3S,4S,5R)-3-(4-fluoro-2-methoxy-3-
(methoxymethyl)phenyl)-4,5-dimethyl-5-
(trifluoromethyl)tetrahydrofuran-2-
carboxamido)picolinamide

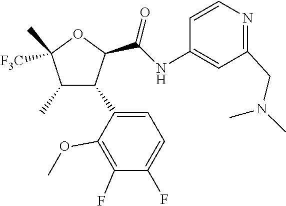 [SMILES: COc1c([C@H]2[C@H](C(=O)Nc3ccnc(CN(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((dimethylamino)methyl)pyridin-4-yl)-4,5-
dimethyl-5-(trifluoromethyl)tetrahydrofuran-2-
carboxamide

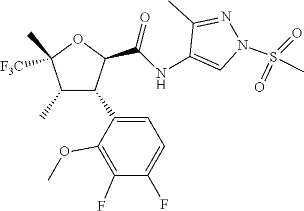 [SMILES: COc1c([C@H]2[C@H](C(=O)Nc3cn(S(C)(=O)=O)nc3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(3-methyl-1-
(methylsulfonyl)-1H-pyrazol-4-yl)-5-
(trifluoromethyl)tetrahydrofuran-2-carboxamide

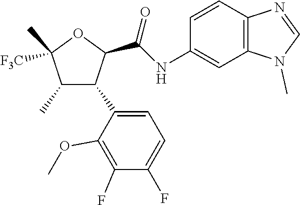 [SMILES: COc1c([C@H]2[C@H](C(=O)Nc3ccc4ncn(C)c4c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(1-methyl-1H-
benzo[d]imidazol-6-yl)-5-
(trifluoromethyl)tetrahydrofuran-2-carboxamide

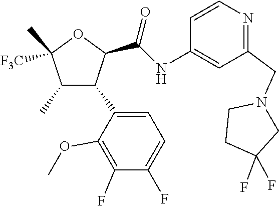 [SMILES: COc1c([C@H]2[C@H](C(=O)Nc3ccnc(CN4CCC(F)(F)C4)c3)O[C@@](C)(C(F)(F)F)[C@H]2C)ccc(F)c1F]

(2R,3S,4S,5R)-3-(3,4-difluoro-2-
methoxyphenyl)-N-(2-((3,3-difluoropyrrolidin-1-
yl)methyl)pyridin-4-yl)-4,5-dimethyl-5-
(trifluoromethyl)tetrahydrofuran-2-carboxamide

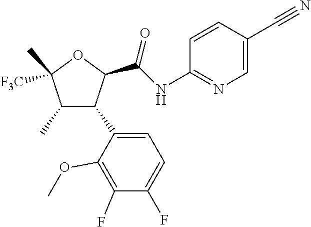 [SMILES: COc1c([C@H]2[C@H](C(=O)Nc3ccc(C#N)cn3)O[C@@](C)(C(F)(F)F)[C@H]2C)ccc(F)c1F]

(2R,3S,4S,5R)-N-(5-cyanopyridin-2-yl)-3-(3,4-
difluoro-2-methoxyphenyl)-4,5-dimethyl-5-
(trifluoromethyl)tetrahydrofuran-2-carboxamide

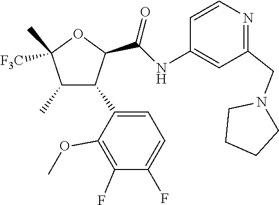 [SMILES: COc1c([C@H]2[C@H](C(=O)Nc3ccnc(CN4CCCC4)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(pyrrolidin-
1-ylmethyl)pyridin-4-yl)-5-
(trifluoromethyl)tetrahydrofuran-2-carboxamide

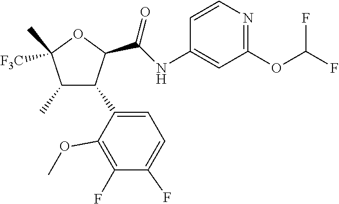 [SMILES: COc1c([C@H]2[C@H](C(=O)Nc3ccnc(OC(F)F)c3)O[C@@](C)(C(F)(F)F)[C@H]2C)ccc(F)c1F]

(2R,3S,4S,5R)-3-(3,4-difluoro-2-methoxyphenyl)-
N-(2-(difluoromethoxy)pyridin-4-yl)-4,5-dimethyl-
5-(trifluoromethyl)tetrahydrofuran-2-carboxamide

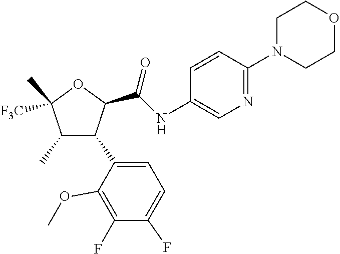 [SMILES: COc1c([C@H]2[C@H](C(=O)Nc3ccc(N4CCOCC4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(1-methyl-1H-
benzo[d]imidazol-6-yl)-5-
(trifluoromethyl)tetrahydrofuran-2-carboxamide

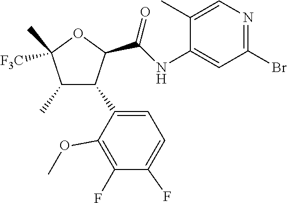 [SMILES: COc1c([C@H]2[C@H](C(=O)Nc3cc(Br)ncc3C)O[C@@](C)(C(F)(F)F)[C@H]2C)ccc(F)c1F]

(2R,3S,4S,5R)-N-(2-bromo-5-methylpyridin-4-yl)-
3-(3,4-difluoro-2-methoxyphenyl)-4,5-dimethyl-5-
(trifluoromethyl)tetrahydrofuran-2-carboxamide

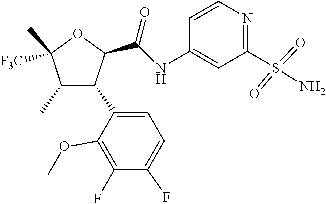 [SMILES: COc1c([C@H]2[C@H](C(=O)Nc3ccnc(S(N)(=O)=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-sulfamoylpyridin-4-yl)-5-
(trifluoromethyl)tetrahydrofuran-2-carboxamide

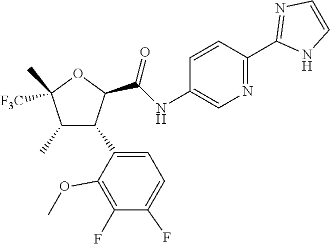 [SMILES: COc1c([C@H]2[C@H](C(=O)Nc3ccc(-c4ncc[nH]4)nc3)O[C@@](C)(C(F)(F)F)[C@H]2C)ccc(F)c1F]

(2R,3S,4S,5R)-N-(6-(1H-imidazol-2-yl)pyridin-
3-yl)-3-(3,4-difluoro-2-methoxyphenyl)-4,5-
dimethyl-5-(trifluoromethyl)tetrahydrofuran-2-
carboxamide

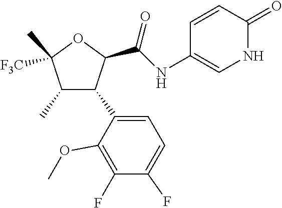 [SMILES: COc1c([C@H]2[C@H](C(=O)Nc3ccc(=O)[nH]c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(6-oxo-1,6-dihydropyridin-3-yl)-5-
(trifluoromethyl)tetrahydrofuran-2-carboxamide

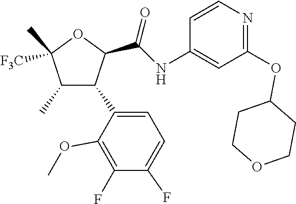 [SMILES: COc1c([C@H]2[C@H](C(=O)Nc3ccnc(OC4CCOCC4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((tetrahydro-2H-pyran-4-
yl)oxy)pyridin-4-yl)-5-
(trifluoromethyl)tetrahydrofuran-2-carboxamide

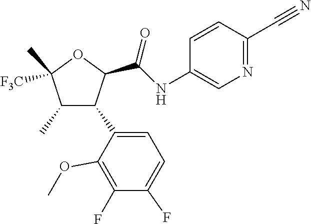 [SMILES: COc1c([C@H]2[C@H](C(=O)Nc3ccc(C#N)nc3)O[C@@](C)(C(F)(F)F)[C@H]2C)ccc(F)c1F]

(2R,3S,4S,5R)-N-(6-cyanopyridin-3-yl)-3-(3,4-
difluoro-2-methoxyphenyl)-4,5-dimethyl-5-
(trifluoromethyl)tetrahydrofuran-2-carboxamide

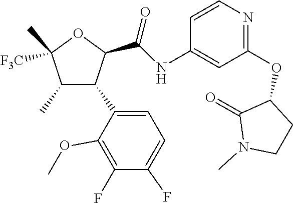 [SMILES: COc1c([C@H]2[C@H](C(=O)Nc3ccnc(O[C@@H]4CCN(C)C4=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(((R)-1-methyl-2-oxopyrrolidin-
3-yl)oxy)pyridin-4-yl)-5-
(trifluoromethyl)tetrahydrofuran-2-carboxamide

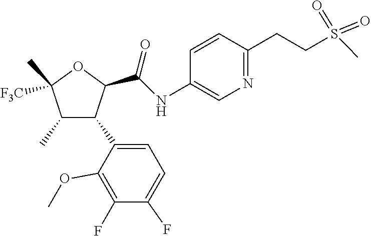 [SMILES: COc1c([C@H]2[C@H](C(=O)Nc3ccc(CCS(C)(=O)=O)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(2-
(methylsulfonyl)ethyl)pyridin-3-yl)-5-
(trifluoromethyl)tetrahydrofuran-2-carboxamide

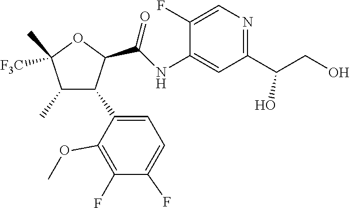 [SMILES: COc1c([C@H]2[C@H](C(=O)Nc3cc([C@@H](O)CO)ncc3F)O[C@@](C)(C(F)(F)F)[C@H]2C)ccc(F)c1F]

(2R,3S,4S,5R)-3-(3,4-difluoro-2-methoxyphenyl)-
N-(2-((R)-1,2-dihydroxyethyl)-5-fluoropyridin-4-
yl)-4,5-dimethyl-5-
(trifluoromethyl)tetrahydrofuran-2-carboxamide

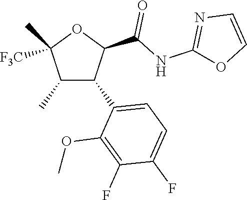 [SMILES: COc1c([C@H]2[C@H](C(=O)Nc3ncco3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(oxazol-2-yl)-5-
(trifluoromethyl)tetrahydrofuran-2-carboxamide

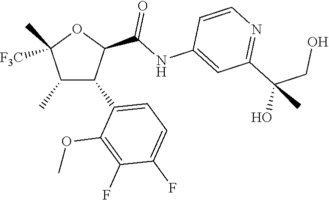 [SMILES: COc1c([C@H]2[C@H](C(=O)Nc3ccnc([C@@](C)(O)CO)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1,2-dihydroxypropan-2-yl)pyridin-4-yl)-
4,5-dimethyl-5-(trifluoromethyl)tetrahydrofuran-2-
carboxamide

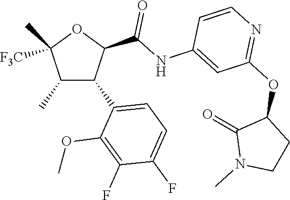 [SMILES: COc1c([C@H]2[C@H](C(=O)Nc3ccnc(O[C@H]4CCN(C)C4=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(((S)-1-methyl-2-oxopyrrolidin-
3-yl)oxy)pyridin-4-yl)-5-
(trifluoromethyl)tetrahydrofuran-2-carboxamide

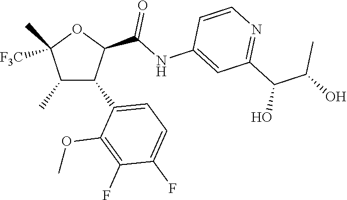 [SMILES: COc1c([C@H]2[C@H](C(=O)Nc3ccnc([C@@H](O)[C@H](C)O)c3)O[C@@](C)(C(F)(F)F)[C@H]2C)ccc(F)c1F]

(2R,3S,4S,5R)-3-(3,4-difluoro-2-methoxyphenyl)-
N-(2-((1R,2S)-1,2-dihydroxypropyl)pyridin-4-yl)-
4,5-dimethyl-5-(trifluoromethyl)tetrahydrofuran-2-
carboxamide

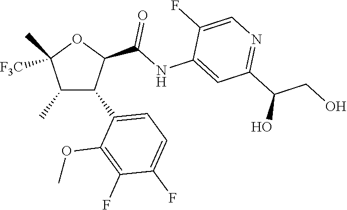 [SMILES: COc1c([C@H]2[C@H](C(=O)Nc3cc([C@H](O)CO)ncc3F)O[C@@](C)(C(F)(F)F)[C@H]2C)ccc(F)c1F]

(2R,3S,4S,5R)-3-(3,4-difluoro-2-methoxyphenyl)-
N-(2-((S)-1,2-dihydroxyethyl)-5-fluoropyridin-4-
yl)-4,5-dimethyl-5-
(trifluoromethyl)tetrahydrofuran-2-carboxamide

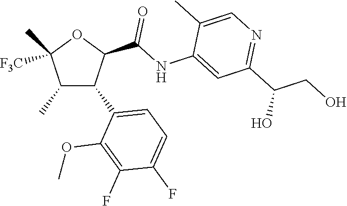 [SMILES: COc1c([C@H]2[C@H](C(=O)Nc3cc([C@@H](O)CO)ncc3C)O[C@@](C)(C(F)(F)F)[C@H]2C)ccc(F)c1F]

(2R,3S,4S,5R)-3-(3,4-difluoro-2-methoxyphenyl)-
N-(2-((R)-1,2-dihydroxyethyl)-5-methylpyridin-4-
yl)-4,5-dimethyl-5-(trifluoromethyl)tetrahydrofuran-
2-carboxamide

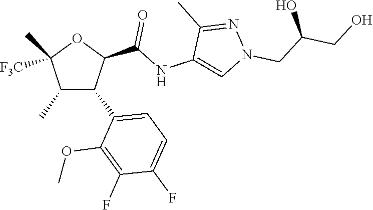 [SMILES: COc1c([C@H]2[C@H](C(=O)Nc3cn(C[C@@H](O)CO)nc3C)O[C@@](C)(C(F)(F)F)[C@H]2C)ccc(F)c1F]

(2R,3S,4S,5R)-3-(3,4-difluoro-2-methoxyphenyl)-
N-(1-((R)-2,3-dihydroxypropyl)-3-methyl-1H-
pyrazol-4-yl)-4,5-dimethyl-5-
(trifluoromethyl)tetrahydrofuran-2-carboxamide

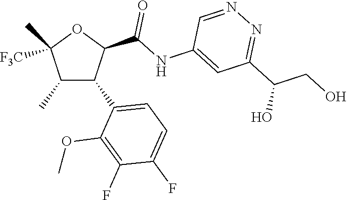 [SMILES: COc1c([C@H]2[C@H](C(=O)Nc3cnnc([C@@H](O)CO)c3)O[C@@](C)(C(F)(F)F)[C@H]2C)ccc(F)c1F]

(2R,3S,4S,5R)-3-(3,4-difluoro-2-methoxyphenyl)-
N-(6-((R)-1,2-dihydroxyethyl)pyridazin-4-yl)-4,5-
dimethyl-5-(trifluoromethyl)tetrahydrofuran-2-
carboxamide

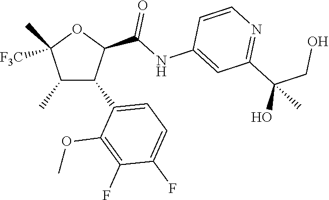 [SMILES: COc1c([C@H]2[C@H](C(=O)Nc3ccnc([C@](C)(O)CO)c3)O[C@@](C)(C(F)(F)F)[C@H]2C)ccc(F)c1F]

(2R,3S,4S,5R)-3-(3,4-difluoro-2-methoxyphenyl)-
N-(2-((S)-1,2-dihydroxypropan-2-yl)pyridin-4-yl)-
4,5-dimethyl-5-(trifluoromethyl)tetrahydrofuran-2-
carboxamide

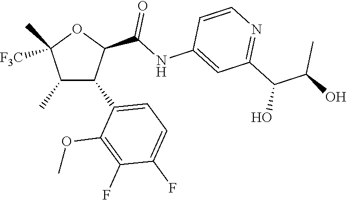 [SMILES: COc1c([C@H]2[C@H](C(=O)Nc3ccnc([C@@H](O)[C@@H](C)O)c3)O[C@@](C)(C(F)(F)F)[C@H]2C)ccc(F)c1F]

(2R,3S,4S,5R)-3-(3,4-difluoro-2-methoxyphenyl)-
N-(2-((1R,2R)-1,2-dihydroxypropyl)pyridin-4-yl)-
4,5-dimethyl-5-(trifluoromethyl)tetrahydrofuran-2-
carboxamide

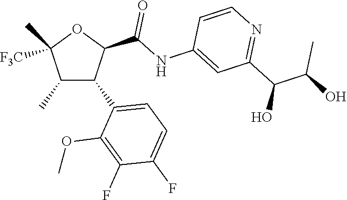 [SMILES: COc1c([C@H]2[C@H](C(=O)Nc3ccnc([C@H](O)[C@@H](C)O)c3)O[C@@](C)(C(F)(F)F)[C@H]2C)ccc(F)c1F]

(2R,3S,4S,5R)-3-(3,4-difluoro-2-methoxyphenyl)-
N-(2-((1S,2R)-1,2-dihydroxypropyl)pyridin-4-yl)-
4,5-dimethyl-5-(trifluoromethyl)tetrahydrofuran-2-
carboxamide

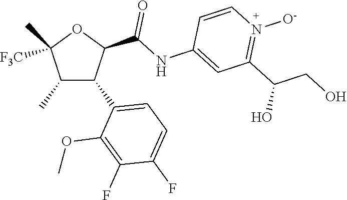

4-((2R,3S,4S,5R)-3-(3,4-difluoro-2-
methoxyphenyl)-4,5-dimethyl-5-
(trifluoromethyl)tetrahydrofuran-2-carboxamido)-2-
((R)-1,2-dihydroxyethyl)pyridine 1-oxide

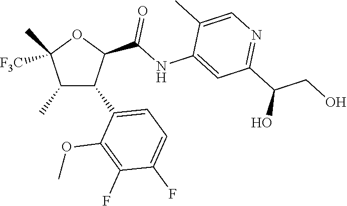 [SMILES: COc1c([C@H]2[C@H](C(=O)Nc3cc([C@H](O)CO)ncc3C)O[C@@](C)(C(F)(F)F)[C@H]2C)ccc(F)c1F]

(2R,3S,4S,5R)-3-(3,4-difluoro-2-methoxyphenyl)-
N-(2-((S)-1,2-dihydroxyethyl)-5-methylpyridin-4-
yl)-4,5-dimethyl-5-
(trifluoromethyl)tetrahydrofuran-2-carboxamide

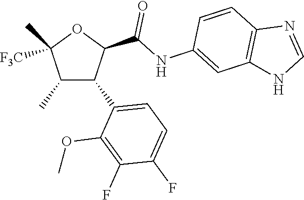 [SMILES: COc1c([C@H]2[C@H](C(=O)Nc3ccc4nc[nH]c4c3)O[C@@](C)(C(F)(F)F)[C@H]2C)ccc(F)c1F]

(2R,3S,4S,5R)-N-(1H-benzo[d]imidazol-6-yl)-3-
(3,4-difluoro-2-methoxyphenyl)-4,5-dimethyl-5-
(trifluoromethyl)tetrahydrofuran-2-carboxamide

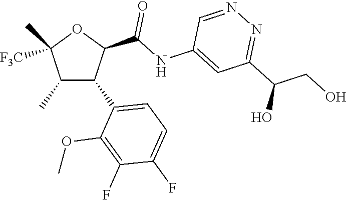 [SMILES: COc1c([C@H]2[C@H](C(=O)Nc3cnnc([C@H](O)CO)c3)O[C@@](C)(C(F)(F)F)[C@H]2C)ccc(F)c1F]

(2R,3S,4S,5R)-3-(3,4-difluoro-2-methoxyphenyl)-
N-(6-((S)-1,2-dihydroxyethyl)pyridazin-4-yl)-4,5-
dimethyl-5-(trifluoromethyl)tetrahydrofuran-2-
carboxamide

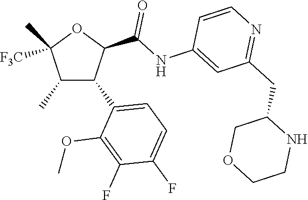 [SMILES: COc1c([C@H]2[C@H](C(=O)Nc3ccnc(C[C@H]4COCCN4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(((S)-morpholin-3-
yl)methyl)pyridin-4-yl)-5-
(trifluoromethyl)tetrahydrofuran-2-carboxamide

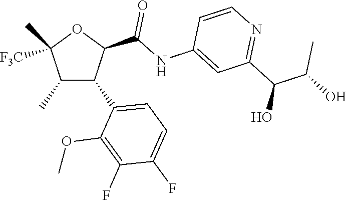 [SMILES: COc1c([C@H]2[C@H](C(=O)Nc3ccnc([C@H](O)[C@H](C)O)c3)O[C@@](C)(C(F)(F)F)[C@H]2C)ccc(F)c1F]

(2R,3S,4S,5R)-3-(3,4-difluoro-2-methoxyphenyl)-
N-(2-((1S,2S)-1,2-dihydroxypropyl)pyridin-4-yl)-
4,5-dimethyl-5-(trifluoromethyl)tetrahydrofuran-2-
carboxamide

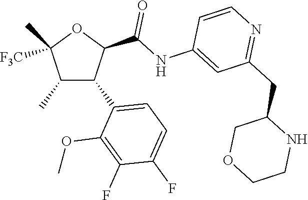 [SMILES: COc1c([C@H]2[C@H](C(=O)Nc3ccnc(C[C@@H]4COCCN4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(((R)-morpholin-3-
yl)methyl)pyridin-4-yl)-5-
(trifluoromethyl)tetrahydrofuran-2-carboxamide

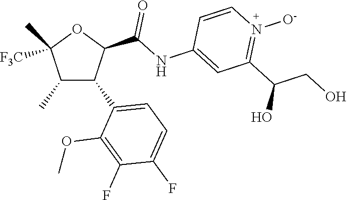

4-((2R,3S,4S,5R)-3-(3,4-difluoro-2-methoxyphenyl)-
4,5-dimethyl-5-(trifluoromethyl)tetrahydrofuran-2-
carboxamido)-2-((S)-1,2-dihydroxyethyl)pyridine 1-
oxide

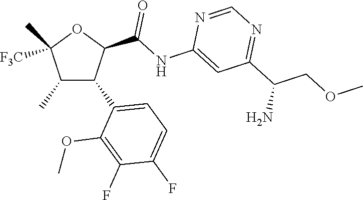 [SMILES: COC[C@H](N)c1cc(NC(=O)[C@@H]2O[C@@](C)(C(F)(F)F)[C@@H](C)[C@H]2c2ccc(F)c(F)c2OC)ncn1]

(2R,3S,4S,5R)-N-(6-((R)-1-amino-2-
methoxyethyl)pyrimidin-4-yl)-3-(3,4-difluoro-2-
methoxyphenyl)-4,5-dimethyl-5-
(trifluoromethyl)tetrahydrofuran-2-carboxamide

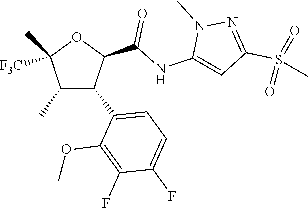 [SMILES: COc1c([C@H]2[C@H](C(=O)Nc3cc(S(C)(=O)=O)nn3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(1-methyl-3-
(methylsulfonyl)-1H-pyrazol-5-yl)-5-
(trifluoromethyl)tetrahydrofuran-2-carboxamide

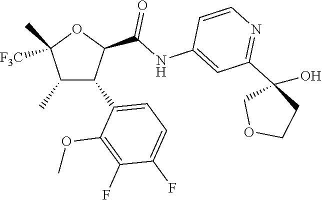 [SMILES: COc1c([C@H]2[C@H](C(=O)Nc3ccnc([C@@]4(O)CCOC4)c3)O[C@@](C)(C(F)(F)F)[C@H]2C)ccc(F)c1F]

(2R,3S,4S,5R)-3-(3,4-difluoro-2-methoxyphenyl)-
N-(2-((S)-3-hydroxytetrahydrofuran-3-yl)pyridin-
4-yl)-4,5-dimethyl-5-
(trifluoromethyl)tetrahydrofuran-2-carboxamide

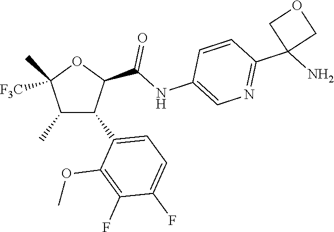 [SMILES: COc1c([C@H]2[C@H](C(=O)Nc3ccc(C4(N)COC4)nc3)O[C@@](C)(C(F)(F)F)[C@H]2C)ccc(F)c1F]

(2R,3S,4S,5R)-N-(6-(3-aminooxetan-3-yl)pyridin-3-
yl)-3-(3,4-difluoro-2-methoxyphenyl)-4,5-dimethyl-5-
(trifluoromethyl)tetrahydrofuran-2-carboxamide

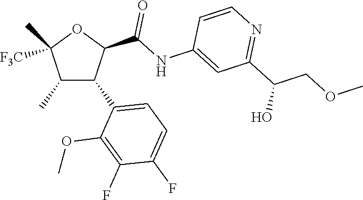 [SMILES: COC[C@H](O)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-((R)-1-hydroxy-2-methoxyethyl)pyridin-4-yl)-
4,5-dimethyl-5-(trifluoromethyl)tetrahydrofuran-2-
carboxamide

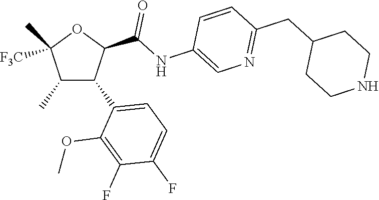 [SMILES: COc1c([C@H]2[C@H](C(=O)Nc3ccc(CC4CCNCC4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(piperidin-4-
ylmethyl)pyridin-3-yl)-5-
(trifluoromethyl)tetrahydrofuran-2-carboxamide

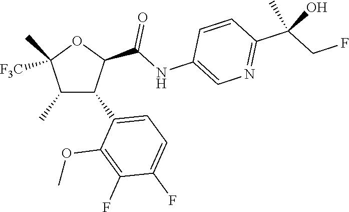 [SMILES: COc1c([C@H]2[C@H](C(=O)Nc3ccc([C@@](C)(O)CF)nc3)O[C@@](C)(C(F)(F)F)[C@H]2C)ccc(F)c1F]

(2R,3S,4S,5R)-3-(3,4-difluoro-2-methoxyphenyl)-
N-(6-((R)-1-fluoro-2-hydroxypropan-2-yl)pyridin-
3-yl)-4,5-dimethyl-5-
(trifluoromethyl)tetrahydrofuran-2-carboxamide

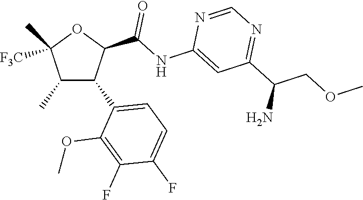 [SMILES: COC[C@@H](N)c1cc(NC(=O)[C@@H]2O[C@@](C)(C(F)(F)F)[C@@H](C)[C@H]2c2ccc(F)c(F)c2OC)ncn1]

(2R,3S,4S,5R)-N-(6-((S)-1-amino-2-
methoxyethyl)pyrimidin-4-yl)-3-(3,4-difluoro-2-
methoxyphenyl)-4,5-dimethyl-5-
(trifluoromethyl)tetrahydrofuran-2-carboxamide

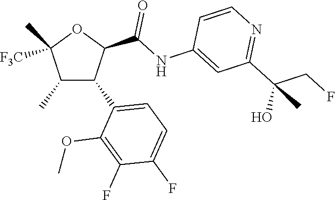 [SMILES: COc1c([C@H]2[C@H](C(=O)Nc3ccnc([C@@](C)(O)CF)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1-fluoro-2-hydroxypropan-2-yl)pyridin-4-
yl)-4,5-dimethyl-5-(trifluoromethyl)tetrahydrofuran-
2-carboxamide

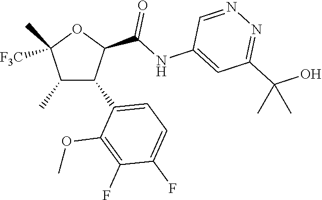 [SMILES: COc1c([C@H]2[C@H](C(=O)Nc3cnnc(C(C)(C)O)c3)O[C@@](C)(C(F)(F)F)[C@H]2C)ccc(F)c1F]

(2R,3S,4S,5R)-3-(3,4-difluoro-2-methoxyphenyl)-
N-(6-(2-hydroxypropan-2-yl)pyridazin-4-yl)-4,5-
dimethyl-5-(trifluoromethyl)tetrahydrofuran-2-
carboxamide

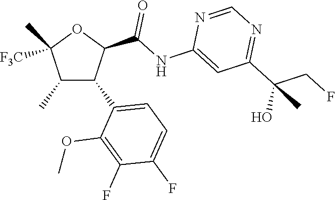 [SMILES: COc1c([C@H]2[C@H](C(=O)Nc3cc([C@@](C)(O)CF)ncn3)O[C@@](C)(C(F)(F)F)[C@H]2C)ccc(F)c1F]

(2R,3S,4S,5R)-3-(3,4-difluoro-2-methoxyphenyl)-
N-(6-((R)-1-fluoro-2-hydroxypropan-2-yl)pyrimidin-
4-yl)-4,5-dimethyl-5-
(trifluoromethyl)tetrahydrofuran-2-carboxamide

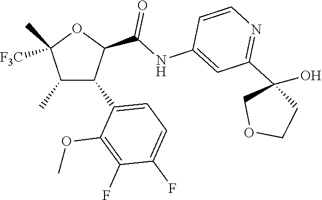 [SMILES: COc1c([C@H]2[C@H](C(=O)Nc3ccnc([C@]4(O)CCOC4)c3)O[C@@](C)(C(F)(F)F)[C@H]2C)ccc(F)c1F]

(2R,3S,4S,5R)-3-(3,4-difluoro-2-methoxyphenyl)-
N-(2-((R)-3-hydroxytetrahydrofuran-3-yl)pyridin-
4-yl)-4,5-dimethyl-5-
(trifluoromethyl)tetrahydrofuran-2-carboxamide

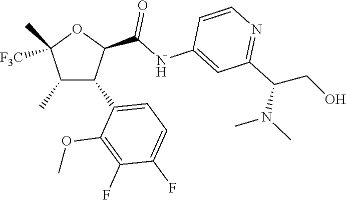 [SMILES: COc1c([C@H]2[C@H](C(=O)Nc3ccnc([C@H](CO)N(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1-(dimethylamino)-2-
hydroxyethyl)pyridin-4-yl)-4,5-dimethyl-5-
(trifluoromethyl)tetrahydrofuran-2-carboxamide

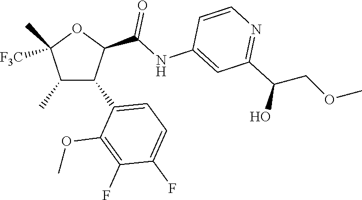 [SMILES: COC[C@@H](O)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-((S)-1-hydroxy-2-methoxyethyl)pyridin-4-yl)-
4,5-dimethyl-5-(trifluoromethyl)tetrahydrofuran-2-
carboxamide

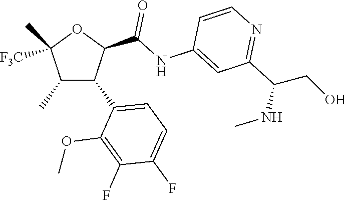 [SMILES: CN[C@@H](CO)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-((R)-2-hydroxy-1-(methylamino)ethyl)pyridin-
4-yl)-4,5-dimethyl-5-
(trifluoromethyl)tetrahydrofuran-2-carboxamide

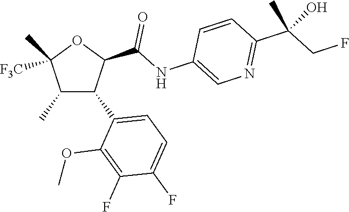 [SMILES: COc1c([C@H]2[C@H](C(=O)Nc3ccc([C@](C)(O)CF)nc3)O[C@@](C)(C(F)(F)F)[C@H]2C)ccc(F)c1F]

(2R,3S,4S,5R)-3-(3,4-difluoro-2-methoxyphenyl)-
N-(6-((S)-1-fluoro-2-hydroxypropan-2-yl)pyridin-
3-yl)-4,5-dimethyl-5-
(trifluoromethyl)tetrahydrofuran-2-carboxamide

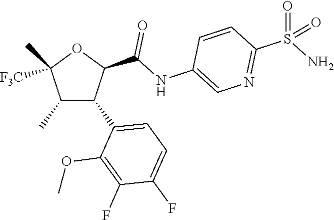 [SMILES: COc1c([C@H]2[C@H](C(=O)Nc3ccc(S(N)(=O)=O)nc3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(6-sulfamoylpyridin-3-yl)-5-
(trifluoromethyl)tetrahydrofuran-2-carboxamide

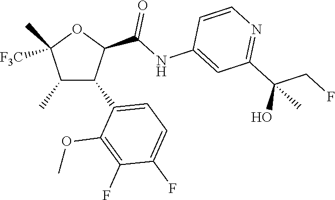 [SMILES: COc1c([C@H]2[C@H](C(=O)Nc3ccnc([C@](C)(O)CF)c3)O[C@@](C)(C(F)(F)F)[C@H]2C)ccc(F)c1F]

(2R,3S,4S,5R)-3-(3,4-difluoro-2-methoxyphenyl)-
N-(2-((S)-1-fluoro-2-hydroxypropan-2-yl)pyridin-
4-yl)-4,5-dimethyl-5-
(trifluoromethyl)tetrahydrofuran-2-carboxamide

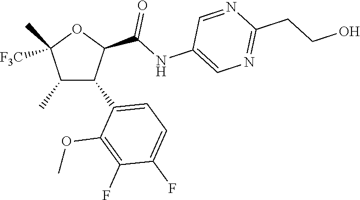 [SMILES: COc1c([C@H]2[C@H](C(=O)Nc3cnc(CCO)nc3)O[C@@](C)(C(F)(F)F)[C@H]2C)ccc(F)c1F]

(2R,3S,4S,5R)-3-(3,4-difluoro-2-methoxyphenyl)-N-(2-
(2-hydroxyethyl)pyrimidin-5-yl)-4,5-dimethyl-5-
(trifluoromethyl)tetrahydrofuran-2-carboxamide

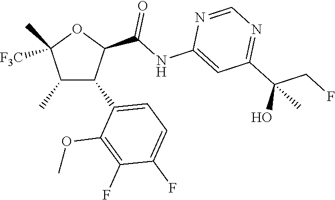 [SMILES: COc1c([C@H]2[C@H](C(=O)Nc3cc([C@](C)(O)CF)ncn3)O[C@@](C)(C(F)(F)F)[C@H]2C)ccc(F)c1F]

(2R,3S,4S,5R)-3-(3,4-difluoro-2-methoxyphenyl)-
N-(6-((S)-1-fluoro-2-hydroxypropan-2-
yl)pyrimidin-4-yl)-4,5-dimethyl-5-
(trifluoromethyl)tetrahydrofuran-2-carboxamide

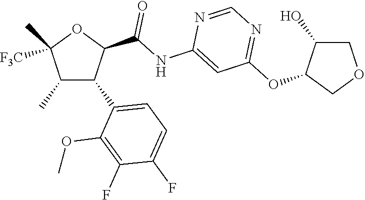 [SMILES: COc1c([C@H]2[C@H](C(=O)Nc3cc(O[C@H]4COC[C@H]4O)ncn3)O[C@@](C)(C(F)(F)F)[C@H]2C)ccc(F)c1F]

(2R,3S,4S,5R)-3-(3,4-difluoro-2-methoxyphenyl)-N-(6-
(((3S,4R)-4-hydroxytetrahydrofuran-3-yl)oxy)pyrimidin-
4-yl)-4,5-dimethyl-5-(trifluoromethyl)tetrahydrofuran-2-
carboxamide

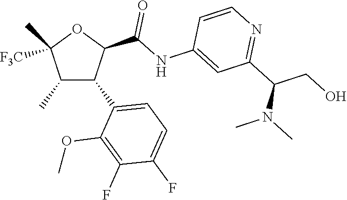 [SMILES: COc1c([C@H]2[C@H](C(=O)Nc3ccnc([C@@H](CO)N(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((S)-1-(dimethylamino)-2-
hydroxyethyl)pyridin-4-yl)-4,5-dimethyl-5-
(trifluoromethyl)tetrahydrofuran-2-carboxamide

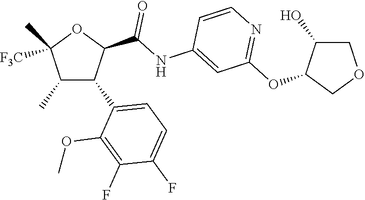 [SMILES: COc1c([C@H]2[C@H](C(=O)Nc3ccnc(O[C@H]4COC[C@H]4O)c3)O[C@@](C)(C(F)(F)F)[C@H]2C)ccc(F)c1F]

(2R,3S,4S,5R)-3-(3,4-difluoro-2-methoxyphenyl)-N-(2-
(((3S,4R)-4-hydroxytetrahydrofuran-3-yl)oxy)pyridin-4-
yl)-4,5-dimethyl-5-(trifluoromethyl)tetrahydrofuran-2-
carboxamide

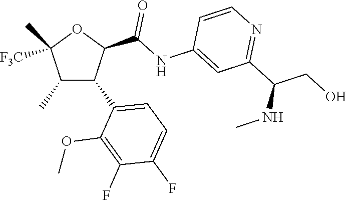 [SMILES: CN[C@H](CO)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-((S)-2-hydroxy-1-
(methylamino)ethyl)pyridin-4-yl)-4,5-dimethyl-5-
(trifluoromethyl)tetrahydrofuran-2-carboxamide

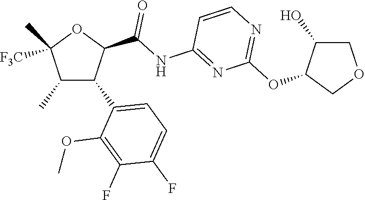 [SMILES: COc1c([C@H]2[C@H](C(=O)Nc3ccnc(O[C@H]4COC[C@H]4O)n3)O[C@@](C)(C(F)(F)F)[C@H]2C)ccc(F)c1F]

(2R,3S,4S,5R)-3-(3,4-difluoro-2-methoxyphenyl)-N-(2-
(((3S,4R)-4-hydroxytetrahydrofuran-3-yl)oxy)pyrimidin-
4-yl)-4,5-dimethyl-5-(trifluoromethyl)tetrahydrofuran-2-
carboxamide

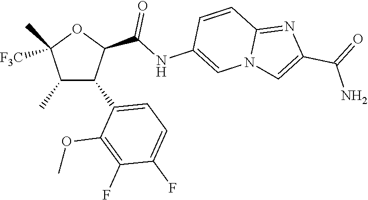 [SMILES: COc1c([C@H]2[C@H](C(=O)Nc3ccc4nc(C(N)=O)cn4c3)O[C@@](C)(C(F)(F)F)[C@H]2C)ccc(F)c1F]

6-((2R,3S,4S,5R)-3-(3,4-difluoro-2-methoxyphenyl)-4,5-
dimethyl-5-(trifluoromethyl)tetrahydrofuran-2-
carboxamido)imidazo[1,2-a]pyridine-2-carboxamide

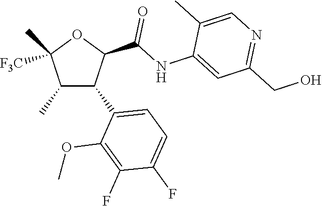 [SMILES: COc1c([C@H]2[C@H](C(=O)Nc3cc(CO)ncc3C)O[C@@](C)(C(F)(F)F)[C@H]2C)ccc(F)c1F]

(2R,3S,4S,5R)-3-(3,4-difluoro-2-methoxyphenyl)-N-(2-
(hydroxymethyl)-5-methylpyridin-4-yl)-4,5-dimethyl-5-
(trifluoromethyl)tetrahydrofuran-2-carboxamide

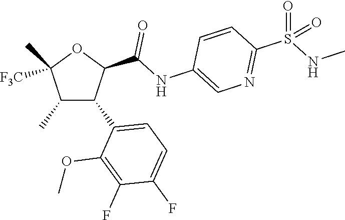 [SMILES: CNS(=O)(=O)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-4,5-
dimethyl-N-(6-(N-methylsulfamoyl)pyridin-3-yl)-5-
(trifluoromethyl)tetrahydrofuran-2-carboxamide

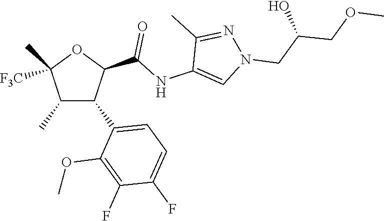 [SMILES: COC[C@@H](O)Cn1cc(NC(=O)[C@@H]2O[C@@](C)(C(F)(F)F)[C@@H](C)[C@H]2c2ccc(F)c(F)c2OC)c(C)n1]

(2R,3S,4S,5R)-3-(3,4-difluoro-2-methoxyphenyl)-
N-(1-((S)-2-hydroxy-3-methoxypropyl)-3-methyl-
1H-pyrazol-4-yl)-4,5-dimethyl-5-
(trifluoromethyl)tetrahydrofuran-2-carboxamide

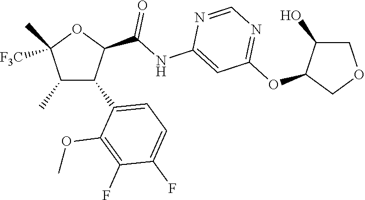 [SMILES: COc1c([C@H]2[C@H](C(=O)Nc3cc(O[C@@H]4COC[C@@H]4O)ncn3)O[C@@](C)(C(F)(F)F)[C@H]2C)ccc(F)c1F]

(2R,3S,4S,5R)-3-(3,4-difluoro-2-methoxyphenyl)-N-(6-
(((3R,4S)-4-hydroxytetrahydrofuran-3-yl)oxy)pyrimidin-
4-yl)-4,5-dimethyl-5-(trifluoromethyl)tetrahydrofuran-2-
carboxamide

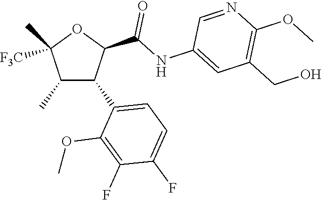 [SMILES: COc1ncc(NC(=O)[C@@H]2O[C@@](C)(C(F)(F)F)[C@@H](C)[C@H]2c2ccc(F)c(F)c2OC)cc1CO]

(2R,3S,4S,5R)-3-(3,4-difluoro-2-methoxyphenyl)-N-(5-
(hydroxymethyl)-6-methoxypyridin-3-yl)-4,5-dimethyl-5-
(trifluoromethyl)tetrahydrofuran-2-carboxamide

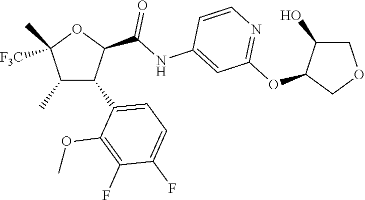 [SMILES: COc1c([C@H]2[C@H](C(=O)Nc3ccnc(O[C@@H]4COC[C@@H]4O)c3)O[C@@](C)(C(F)(F)F)[C@H]2C)ccc(F)c1F]

(2R,3S,4S,5R)-3-(3,4-difluoro-2-methoxyphenyl)-N-(2-
(((3R,4S)-4-hydroxytetrahydrofuran-3-yl)oxy)pyridin-4-
yl)-4,5-dimethyl-5-(trifluoromethyl)tetrahydrofuran-2-
carboxamide

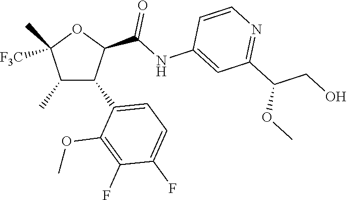 [SMILES: COc1c([C@H]2[C@H](C(=O)Nc3ccnc([C@H](CO)OC)c3)O[C@@](C)(C(F)(F)F)[C@H]2C)ccc(F)c1F]

(2R,3S,4S,5R)-3-(3,4-difluoro-2-methoxyphenyl)-
N-(2-((R)-2-hydroxy-1-methoxyethyl)pyridin-4-yl)-
4,5-dimethyl-5-(trifluoromethyl)tetrahydrofuran-2-
carboxamide

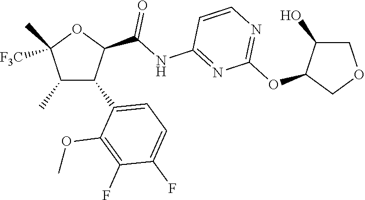 [SMILES: COc1c([C@H]2[C@H](C(=O)Nc3ccnc(O[C@@H]4COC[C@@H]4O)n3)O[C@@](C)(C(F)(F)F)[C@H]2C)ccc(F)c1F]

(2R,3S,4S,5R)-3-(3,4-difluoro-2-methoxyphenyl)-N-(2-
(((3R,4S)-4-hydroxytetrahydrofuran-3-yl)oxy)pyrimidin-
4-yl)-4,5-dimethyl-5-(trifluoromethyl)tetrahydrofuran-2-
carboxamide

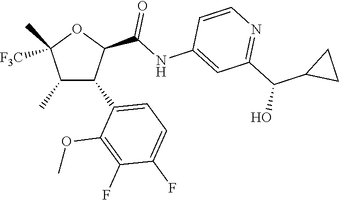 [SMILES: COc1c([C@H]2[C@H](C(=O)Nc3ccnc([C@@H](O)C4CC4)c3)O[C@@](C)(C(F)(F)F)[C@H]2C)ccc(F)c1F]

(2R,3S,4S,5R)-N-(2-((S)-
cyclopropyl(hydroxy)methyl)pyridin-4-yl)-3-(3,4-
difluoro-2-methoxyphenyl)-4,5-dimethyl-5-
(trifluoromethyl)tetrahydrofuran-2-carboxamide

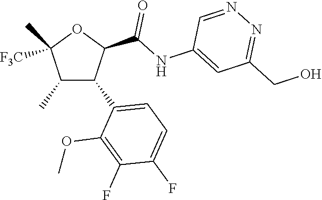 [SMILES: COc1c([C@H]2[C@H](C(=O)Nc3cnnc(CO)c3)O[C@@](C)(C(F)(F)F)[C@H]2C)ccc(F)c1F]

(2R,3S,4S,5R)-3-(3,4-difluoro-2-methoxyphenyl)-N-(6-
(hydroxymethyl)pyridazin-4-yl)-4,5-dimethyl-5-
(trifluoromethyl)tetrahydrofuran-2-carboxamide

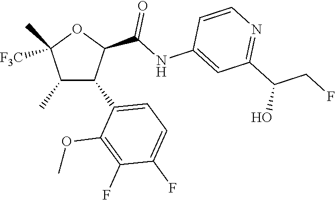 [SMILES: COc1c([C@H]2[C@H](C(=O)Nc3ccnc([C@@H](O)CF)c3)O[C@@](C)(C(F)(F)F)[C@H]2C)ccc(F)c1F]

(2R,3S,4S,5R)-3-(3,4-difluoro-2-methoxyphenyl)-
N-(2-((R)-2-fluoro-1-hydroxyethyl)pyridin-4-yl)-4,5-
dimethyl-5-(trifluoromethyl)tetrahydrofuran-2-
carboxamide

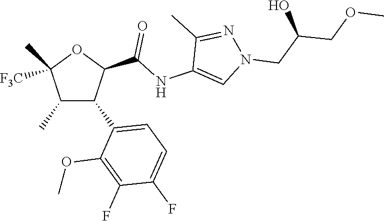 [SMILES: COC[C@H](O)Cn1cc(NC(=O)[C@@H]2O[C@@](C)(C(F)(F)F)[C@@H](C)[C@H]2c2ccc(F)c(F)c2OC)c(C)n1]

(2R,3S,4S,5R)-3-(3,4-difluoro-2-methoxyphenyl)-
N-(1-((R)-2-hydroxy-3-methoxypropyl)-3-methyl-
1H-pyrazol-4-yl)-4,5-dimethyl-5-
(trifluoromethyl)tetrahydrofuran-2-carboxamide

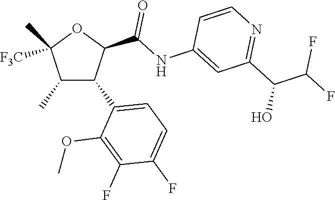 [SMILES: COc1c([C@H]2[C@H](C(=O)Nc3ccnc([C@@H](O)C(F)F)c3)O[C@@](C)(C(F)(F)F)[C@H]2C)ccc(F)c1F]

(2R,3S,4S,5R)-N-(2-((R)-2,2-difluoro-1-
hydroxyethyl)pyridin-4-yl)-3-(3,4-difluoro-2-
methoxyphenyl)-4,5-dimethyl-5-
(trifluoromethyl)tetrahydrofuran-2-carboxamide

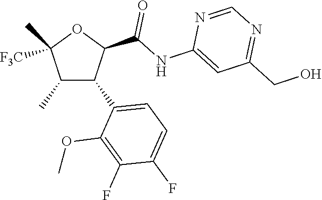 [SMILES: COc1c([C@H]2[C@H](C(=O)Nc3cc(CO)ncn3)O[C@@](C)(C(F)(F)F)[C@H]2C)ccc(F)c1F]

(2R,3S,4S,5R)-3-(3,4-difluoro-2-methoxyphenyl)-N-(6-
(hydroxymethyl)pyrimidin-4-yl)-4,5-dimethyl-5-
(trifluoromethyl)tetrahydrofuran-2-carboxamide

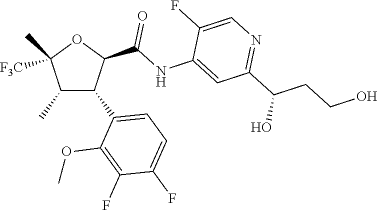 [SMILES: COc1c([C@H]2[C@H](C(=O)Nc3cc([C@@H](O)CCO)ncc3F)O[C@@](C)(C(F)(F)F)[C@H]2C)ccc(F)c1F]

(2R,3S,4S,5R)-3-(3,4-difluoro-2-methoxyphenyl)-
N-(2-((S)-1,3-dihydroxypropyl)-5-fluoropyridin-4-
yl)-4,5-dimethyl-5-(trifluoromethyl)tetrahydrofuran-
2-carboxamide

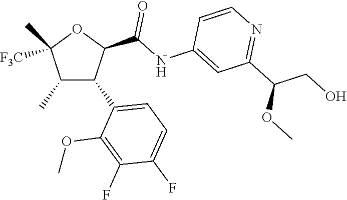 [SMILES: COc1c([C@H]2[C@H](C(=O)Nc3ccnc([C@@H](CO)OC)c3)O[C@@](C)(C(F)(F)F)[C@H]2C)ccc(F)c1F]

(2R,3S,4S,5R)-3-(3,4-difluoro-2-methoxyphenyl)-
N-(2-((S)-2-hydroxy-1-methoxyethyl)pyridin-4-yl)-
4,5-dimethyl-5-(trifluoromethyl)tetrahydrofuran-2-
carboxamide

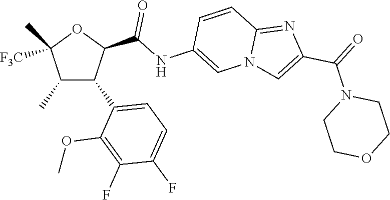 [SMILES: COc1c([C@H]2[C@H](C(=O)Nc3ccc4nc(C(=O)N5CCOCC5)cn4c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(morpholine-4-carbonyl)imidazo[1,2-
a]pyridin-6-yl)-5-(trifluoromethyl)tetrahydrofuran-2-
carboxamide

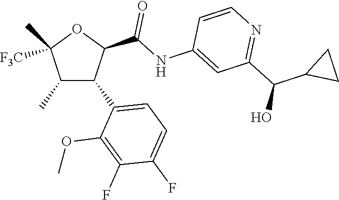 [SMILES: COc1c([C@H]2[C@H](C(=O)Nc3ccnc([C@H](O)C4CC4)c3)O[C@@](C)(C(F)(F)F)[C@H]2C)ccc(F)c1F]

(2R,3S,4S,5R)-N-(2-((R)-
cyclopropyl(hydroxy)methyl)pyridin-4-yl)-3-(3,4-
difluoro-2-methoxyphenyl)-4,5-dimethyl-5-
(trifluoromethyl)tetrahydrofuran-2-carboxamide

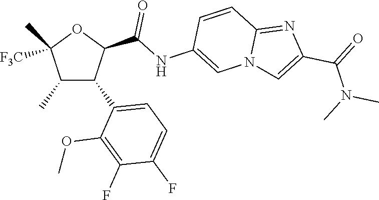 [SMILES: COc1c([C@H]2[C@H](C(=O)Nc3ccc4nc(C(=O)N(C)C)cn4c3)O[C@@](C)(C(F)(F)F)[C@H]2C)ccc(F)c1F]

6-((2R,3S,4S,5R)-3-(3,4-difluoro-2-methoxyphenyl)-
4,5-dimethyl-5-(trifluoromethyl)tetrahydrofuran-2-
carboxamido)-N,N-dimethylimidazo[1,2-a]pyridine-2-
carboxamide

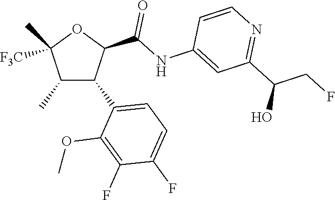 [SMILES: COc1c([C@H]2[C@H](C(=O)Nc3ccnc([C@H](O)CF)c3)O[C@@](C)(C(F)(F)F)[C@H]2C)ccc(F)c1F]

(2R,3S,4S,5R)-3-(3,4-difluoro-2-methoxyphenyl)-
N-(2-((S)-2-fluoro-1-hydroxyethyl)pyridin-4-yl)-
4,5-dimethyl-5-(trifluoromethyl)tetrahydrofuran-2-
carboxamide

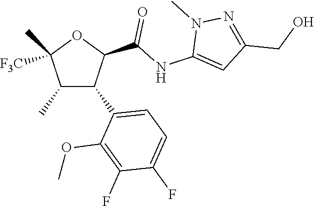 [SMILES: COc1c([C@H]2[C@H](C(=O)Nc3cc(CO)nn3C)O[C@@](C)(C(F)(F)F)[C@H]2C)ccc(F)c1F]

(2R,3S,4S,5R)-3-(3,4-difluoro-2-methoxyphenyl)-
N-(3-(hydroxymethyl)-1-methyl-1H-pyrazol-5-yl)-
4,5-dimethyl-5-(trifluoromethyl)tetrahydrofuran-2-
carboxamide

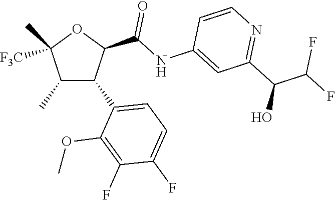 [SMILES: COc1c([C@H]2[C@H](C(=O)Nc3ccnc([C@H](O)C(F)F)c3)O[C@@](C)(C(F)(F)F)[C@H]2C)ccc(F)c1F]

(2R,3S,4S,5R)-N-(2-((S)-2,2-difluoro-1-
hydroxyethyl)pyridin-4-yl)-3-(3,4-difluoro-2-
methoxyphenyl)-4,5-dimethyl-5-
(trifluoromethyl)tetrahydrofuran-2-carboxamide

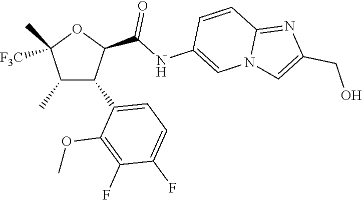 [SMILES: COc1c([C@H]2[C@H](C(=O)Nc3ccc4nc(CO)cn4c3)O[C@@](C)(C(F)(F)F)[C@H]2C)ccc(F)c1F]

(2R,3S,4S,5R)-3-(3,4-difluoro-2-methoxyphenyl)-N-
(2-(hydroxymethyl)imidazo[1,2-a]pyridin-6-yl)-4,5-
dimethyl-5-(trifluoromethyl)tetrahydrofuran-2-
carboxamide

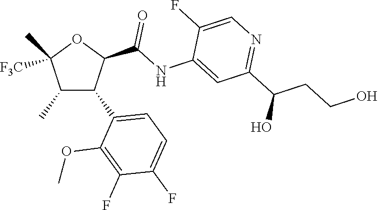 [SMILES: COc1c([C@H]2[C@H](C(=O)Nc3cc([C@H](O)CCO)ncc3F)O[C@@](C)(C(F)(F)F)[C@H]2C)ccc(F)c1F]

(2R,3S,4S,5R)-3-(3,4-difluoro-2-methoxyphenyl)-
N-(2-((R)-1,3-dihydroxypropyl)-5-fluoropyridin-4-
yl)-4,5-dimethyl-5-
(trifluoromethyl)tetrahydrofuran-2-carboxamide

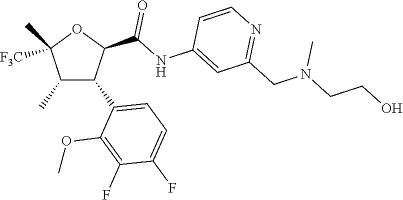 [SMILES: COc1c([C@H]2[C@H](C(=O)Nc3ccnc(CN(C)CCO)c3)O[C@@](C)(C(F)(F)F)[C@H]2C)ccc(F)c1F]

(2R,3S,4S,5R)-3-(3,4-difluoro-2-methoxyphenyl)-N-
(2-(((2-hydroxyethyl)(methyl)amino)methyl)pyridin-4-
yl)-4,5-dimethyl-5-(trifluoromethyl)tetrahydrofuran-2-
carboxamide

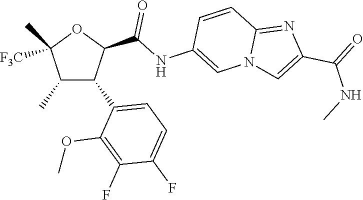 [SMILES: CNC(=O)c1cn2cc(NC(=O)[C@@H]3O[C@@](C)(C(F)(F)F)[C@@H](C)[C@H]3c3ccc(F)c(F)c3OC)ccc2n1]

6-((2R,3S,4S,5R)-3-(3,4-difluoro-2-methoxyphenyl)-4,5-
dimethyl-5-(trifluoromethyl)tetrahydrofuran-2-
carboxamido)-N-methylimidazo[1,2-a]pyridine-2-
carboxamide

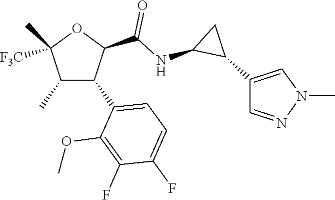 [SMILES: COc1c([C@H]2[C@H](C(=O)N[C@H]3C[C@@H]3c3cnn(C)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-((1S,2R)-2-(1-methyl-1H-pyrazol-4-
yl)cyclopropyl)-5-(trifluoromethyl)tetrahydrofuran-2-
carboxamide

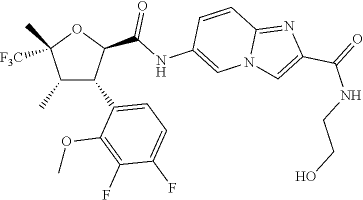 [SMILES: COc1c([C@H]2[C@H](C(=O)Nc3ccc4nc(C(=O)NCCO)cn4c3)O[C@@](C)(C(F)(F)F)[C@H]2C)ccc(F)c1F]

6-((2R,3S,4S,5R)-3-(3,4-difluoro-2-methoxyphenyl)-
4,5-dimethyl-5-(trifluoromethyl)tetrahydrofuran-2-
carboxamido)-N-(2-hydroxyethyl)imidazo[1,2-
a]pyridine-2-carboxamide

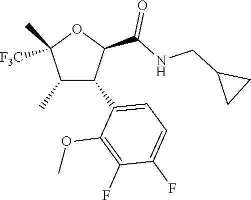 [SMILES: COc1c([C@H]2[C@H](C(=O)NCC3CC3)O[C@@](C)(C(F)(F)F)[C@H]2C)ccc(F)c1F]

(2R,3S,4S,5R)-N-(cyclopropylmethyl)-3-(3,4-
difluoro-2-methoxyphenyl)-4,5-dimethyl-5-
(trifluoromethyl)tetrahydrofuran-2-carboxamide

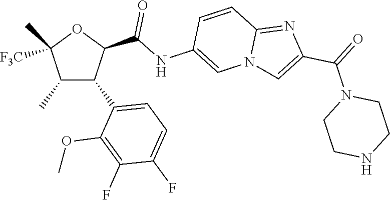 [SMILES: COc1c([C@H]2[C@H](C(=O)Nc3ccc4nc(C(=O)N5CCNCC5)cn4c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(piperazine-1-carbonyl)imidazo[1,2-
a]pyridin-6-yl)-5-(trifluoromethyl)tetrahydrofuran-2-
carboxamide

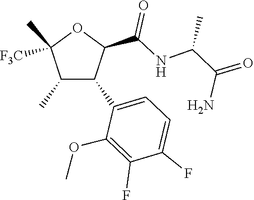 [SMILES: COc1c([C@H]2[C@H](C(=O)N[C@H](C)C(N)=O)O[C@@](C)(C(F)(F)F)[C@H]2C)ccc(F)c1F]

(2R,3S,4S,5R)-N-((R)-1-amino-1-oxopropan-2-
yl)-3-(3,4-difluoro-2-methoxyphenyl)-4,5-
dimethyl-5-(trifluoromethyl)tetrahydrofuran-2-
carboxamide

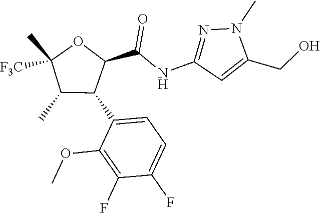 [SMILES: COc1c([C@H]2[C@H](C(=O)Nc3cc(CO)n(C)n3)O[C@@](C)(C(F)(F)F)[C@H]2C)ccc(F)c1F]

(2R,3S,4S,5R)-3-(3,4-difluoro-2-methoxyphenyl)-
N-(5-(hydroxymethyl)-1-methyl-1H-pyrazol-3-yl)-
4,5-dimethyl-5-(trifluoromethyl)tetrahydrofuran-2-
carboxamide

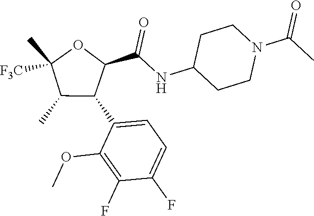 [SMILES: COc1c([C@H]2[C@H](C(=O)NC3CCN(C(C)=O)CC3)O[C@@](C)(C(F)(F)F)[C@H]2C)ccc(F)c1F]

(2R,3S,4S,5R)-N-(1-acetylpiperidin-4-yl)-3-(3,4-
difluoro-2-methoxyphenyl)-4,5-dimethyl-5-
(trifluoromethyl)tetrahydrofuran-2-carboxamide

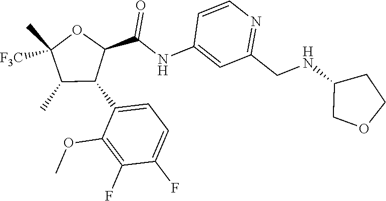 [SMILES: COc1c([C@H]2[C@H](C(=O)Nc3ccnc(CN[C@@H]4CCOC4)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-((((R)-tetrahydrofuran-3-
yl)amino)methyl)pyridin-4-yl)-5-
(trifluoromethyl)tetrahydrofuran-2-carboxamide

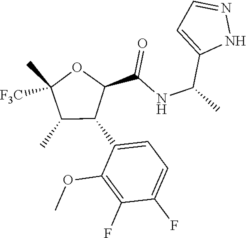 [SMILES: COc1c([C@H]2[C@H](C(=O)N[C@@H](C)c3ccn[nH]3)O[C@@](C)(C(F)(F)F)[C@H]2C)ccc(F)c1F]

(2R,3S,4S,5R)-N-((S)-1-(1H-pyrazol-5-yl)ethyl)-
3-(3,4-difluoro-2-methoxyphenyl)-4,5-dimethyl-5-
(trifluoromethyl)tetrahydrofuran-2-carboxamide

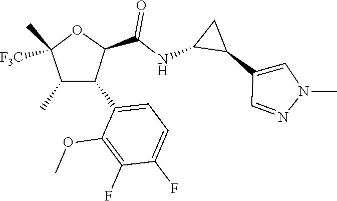 [SMILES: COc1c([C@H]2[C@H](C(=O)N[C@@H]3C[C@H]3c3cnn(C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-((1R,2S)-2-(1-methyl-1H-pyrazol-
4-yl)cyclopropyl)-5-
(trifluoromethyl)tetrahydrofuran-2-carboxamide

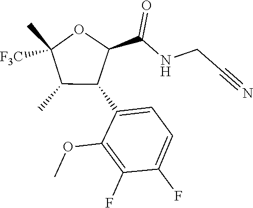 [SMILES: COc1c([C@H]2[C@H](C(=O)NCC#N)O[C@@](C)(C(F)(F)F)[C@H]2C)ccc(F)c1F]

(2R,3S,4S,5R)-N-(cyanomethyl)-3-(3,4-difluoro-2-
methoxyphenyl)-4,5-dimethyl-5-
(trifluoromethyl)tetrahydrofuran-2-carboxamide

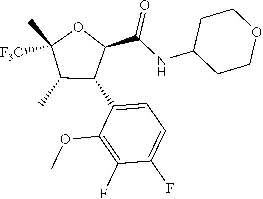 [SMILES: COc1c([C@H]2[C@H](C(=O)NC3CCOCC3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(tetrahydro-2H-
pyran-4-yl)-5-(trifluoromethyl)tetrahydrofuran-2-
carboxamide

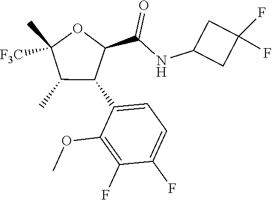 [SMILES: COc1c([C@H]2[C@H](C(=O)NC3CC(F)(F)C3)O[C@@](C)(C(F)(F)F)[C@H]2C)ccc(F)c1F]

(2R,3S,4S,5R)-3-(3,4-difluoro-2-methoxyphenyl)-N-
(3,3-difluorocyclobutyl)-4,5-dimethyl-5-
(trifluoromethyl)tetrahydrofuran-2-carboxamide

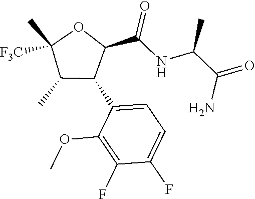 [SMILES: COc1c([C@H]2[C@H](C(=O)N[C@@H](C)C(N)=O)O[C@@](C)(C(F)(F)F)[C@H]2C)ccc(F)c1F]

(2R,3S,4S,5R)-N-((S)-1-amino-1-oxopropan-2-
yl)-3-(3,4-difluoro-2-methoxyphenyl)-4,5-
dimethyl-5-(trifluoromethyl)tetrahydrofuran-2-
carboxamide

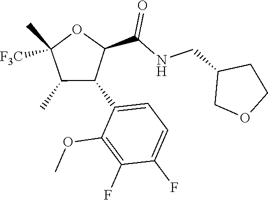 [SMILES: COc1c([C@H]2[C@H](C(=O)NC[C@@H]3CCOC3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(((S)-tetrahydrofuran-3-yl)methyl)-5-
(trifluoromethyl)tetrahydrofuran-2-carboxamide

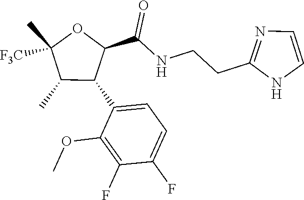 [SMILES: COc1c([C@H]2[C@H](C(=O)NCCc3ncc[nH]3)O[C@@](C)(C(F)(F)F)[C@H]2C)ccc(F)c1F]

(2R,3S,4S,5R)-3-(3,4-difluoro-2-methoxy-phenyl)-
N-[2-(1H-imidazol-2-yl)ethyl]-4,5-dimethyl-5-
(trifluoromethyl)tetrahydrofuran-2-carboxamide

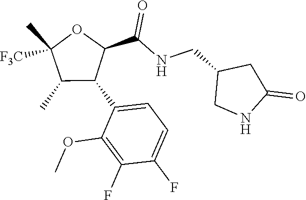 [SMILES: COc1c([C@H]2[C@H](C(=O)NC[C@H]3CNC(=O)C3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(((R)-5-oxopyrrolidin-3-yl)methyl)-5-
(trifluoromethyl)tetrahydrofuran-2-carboxamide

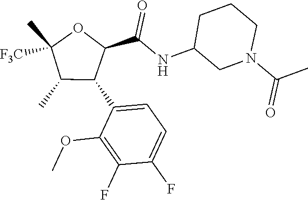 [SMILES: COc1c([C@H]2[C@H](C(=O)NC3CCCN(C(C)=O)C3)O[C@@](C)(C(F)(F)F)[C@H]2C)ccc(F)c1F]

(2R,3S,4S,5R)-N-(1-acetylpiperidin-3-yl)-3-(3,4-
difluoro-2-methoxyphenyl)-4,5-dimethyl-5-
(trifluoromethyl)tetrahydrofuran-2-carboxamide

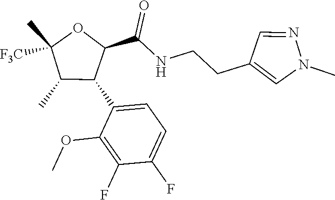 [SMILES: COc1c([C@H]2[C@H](C(=O)NCCc3cnn(C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(1-methyl-1H-pyrazol-4-yl)ethyl)-
5-(trifluoromethyl)tetrahydrofuran-2-carboxamide

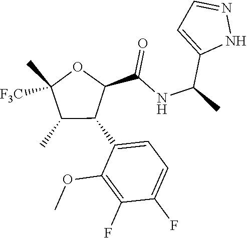 [SMILES: COc1c([C@H]2[C@H](C(=O)N[C@H](C)c3ccn[nH]3)O[C@@](C)(C(F)(F)F)[C@H]2C)ccc(F)c1F]

(2R,3S,4S,5R)-N-((R)-1-(1H-pyrazol-5-yl)ethyl)-3-
(3,4-difluoro-2-methoxyphenyl)-4,5-dimethyl-5-
(trifluoromethyl)tetrahydrofuran-2-carboxamide

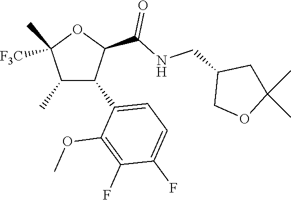 [SMILES: COc1c([C@H]2[C@H](C(=O)NC[C@H]3COC(C)(C)C3)O[C@@](C)(C(F)(F)F)[C@H]2C)ccc(F)c1F]

(2R,3S,4S,5R)-3-(3,4-difluoro-2-methoxyphenyl)-N-(((S)-
5,5-dimethyltetrahydrofuran-3-yl)methyl)-4,5-dimethyl-5-
(trifluoromethyl)tetrahydrofuran-2-carboxamide

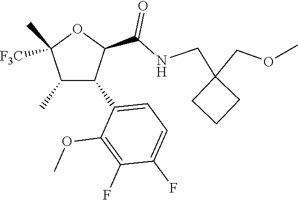 [SMILES: COCC1(CNC(=O)[C@@H]2O[C@@](C)(C(F)(F)F)[C@@H](C)[C@H]2c2ccc(F)c(F)c2OC)CCC1]

(2R,3S,4S,5R)-3-(3,4-difluoro-2-methoxyphenyl)-N-((1-
(methoxymethyl)cyclobutyl)methyl)-4,5-dimethyl-5-
(trifluoromethyl)tetrahydrofuran-2-carboxamide

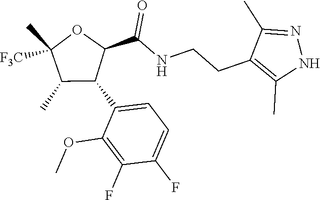 [SMILES: COc1c([C@H]2[C@H](C(=O)NCCc3c(C)n[nH]c3C)O[C@@](C)(C(F)(F)F)[C@H]2C)ccc(F)c1F]

(2R,3S,4S,5R)-3-(3,4-difluoro-2-methoxyphenyl)-N-
(2-(3,5-dimethyl-1H-pyrazol-4-yl)ethyl)-4,5-
dimethyl-5-(trifluoromethyl)tetrahydrofuran-2-
carboxamide

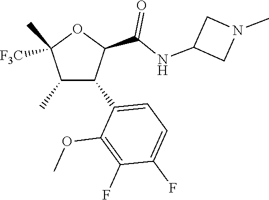 [SMILES: COc1c([C@H]2[C@H](C(=O)NC3CN(C)C3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(1-methylazetidin-3-yl)-5-
(trifluoromethyl)tetrahydrofuran-2-carboxamide

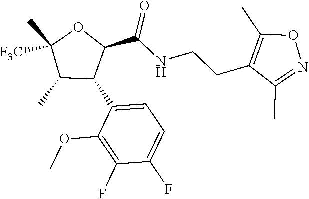

(2R,3S,4S,5R)-3-(3,4-difluoro-2-methoxyphenyl)-N-
(2-(3,5-dimethylisoxazol-4-yl)ethyl)-4,5-dimethyl-5-
(trifluoromethyl)tetrahydrofuran-2-carboxamide

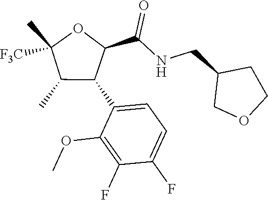 [SMILES: COc1c([C@H]2[C@H](C(=O)NC[C@H]3CCOC3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(((R)-tetrahydrofuran-3-yl)methyl)-5-
(trifluoromethyl)tetrahydrofuran-2-carboxamide

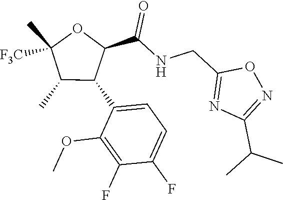 [SMILES: COc1c([C@H]2[C@H](C(=O)NCc3nc(C(C)C)no3)O[C@@](C)(C(F)(F)F)[C@H]2C)ccc(F)c1F]

(2R,3S,4S,5R)-3-(3,4-difluoro-2-methoxyphenyl)-N-((3-isopropyl-
1,2,4-oxadiazol-5-yl)methyl)-4,5-dimethyl-5-
(trifluoromethyl)tetrahydrofuran-2-carboxamide

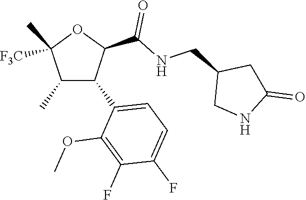 [SMILES: COc1c([C@H]2[C@H](C(=O)NC[C@@H]3CNC(=O)C3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(((S)-5-oxopyrrolidin-3-yl)methyl)-5-
(trifluoromethyl)tetrahydrofuran-2-carboxamide

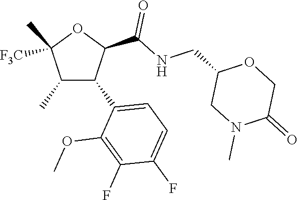 [SMILES: COc1c([C@H]2[C@H](C(=O)NC[C@H]3CN(C)C(=O)CO3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(((S)-4-methyl-5-oxomorpholin-2-yl)methyl)-5-
(trifluoromethyl)tetrahydrofuran-2-carboxamide

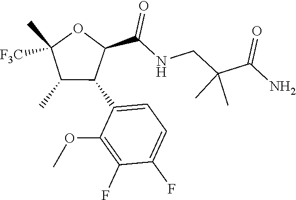 [SMILES: COc1c([C@H]2[C@H](C(=O)NCC(C)(C)C(N)=O)O[C@@](C)(C(F)(F)F)[C@H]2C)ccc(F)c1F]

(2R,3S,4S,5R)-N-(3-amino-2,2-dimethyl-3-oxopropyl)-3-
(3,4-difluoro-2-methoxyphenyl)-4,5-dimethyl-5-
(trifluoromethyl)tetrahydrofuran-2-carboxamide

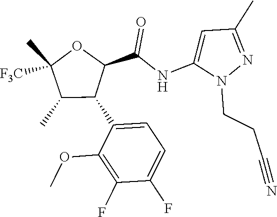 [SMILES: COc1c([C@H]2[C@H](C(=O)Nc3cc(C)nn3CCC#N)O[C@@](C)(C(F)(F)F)[C@H]2C)ccc(F)c1F]

(2R,3S,4S,5R)-N-(1-(2-cyanoethyl)-3-
methyl-1H-pyrazol-5-yl)-3-(3,4-
difluoro-2-methoxyphenyl)-4,5-dimethyl-5-
(trifluoromethyl)tetrahydrofuran-2-carboxamide

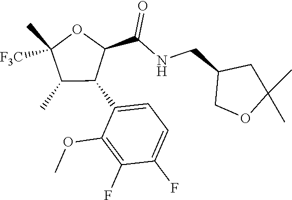 [SMILES: COc1c([C@H]2[C@H](C(=O)NC[C@@H]3COC(C)(C)C3)O[C@@](C)(C(F)(F)F)[C@H]2C)ccc(F)c1F]

(2R,3S,4S,5R)-3-(3,4-difluoro-2-methoxyphenyl)-N-(((R)-
5,5-dimethyltetrahydrofuran-3-yl)methyl)-4,5-dimethyl-5-
(trifluoromethyl)tetrahydrofuran-2-carboxamide

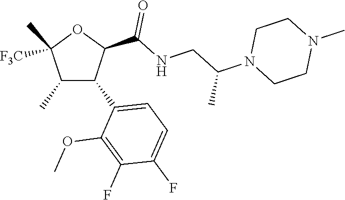 [SMILES: COc1c([C@H]2[C@H](C(=O)NC[C@@H](C)N3CCN(C)CC3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-((R)-2-(4-methylpiperazin-1-
yl)propyl)-5-(trifluoromethyl)tetrahydrofuran-2-
carboxamide

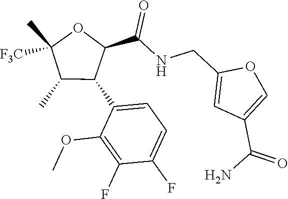 [SMILES: COc1c([C@H]2[C@H](C(=O)NCc3cc(C(N)=O)co3)O[C@@](C)(C(F)(F)F)[C@H]2C)ccc(F)c1F]

5-(((2R,3S,4S,5R)-3-(3,4-difluoro-2-methoxyphenyl)-
4,5-dimethyl-5-(trifluoromethyl)tetrahydrofuran-2-
carboxamido)methyl)furan-3-carboxamide

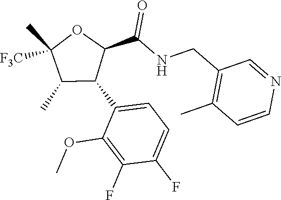 [SMILES: COc1c([C@H]2[C@H](C(=O)NCc3cnccc3C)O[C@@](C)(C(F)(F)F)[C@H]2C)ccc(F)c1F]

(2R,3S,4S,5R)-3-(3,4-difluoro-2-methoxyphenyl)-4,5-
dimethyl-N-((4-methylpyridin-3-yl)methyl)-5-
(trifluoromethyl)tetrahydrofuran-2-carboxamide

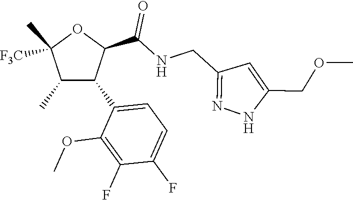 [SMILES: COCc1cc(CNC(=O)[C@@H]2O[C@@](C)(C(F)(F)F)[C@@H](C)[C@H]2c2ccc(F)c(F)c2OC)n[nH]1]

(2R,3S,4S,5R)-3-(3,4-difluoro-2-
methoxyphenyl)-N-((5-(methoxymethyl)-
1H-pyrazol-3-yl)methyl)-4,5-dimethyl-
5-(trifluoromethyl)tetrahydrofuran-2-
carboxamide

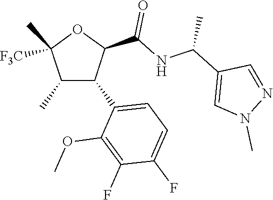 [SMILES: COc1c([C@H]2[C@H](C(=O)N[C@H](C)c3cnn(C)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-((R)-1-(1-methyl-1H-pyrazol-4-yl)ethyl)-5-
(trifluoromethyl)tetrahydrofuran-2-carboxamide

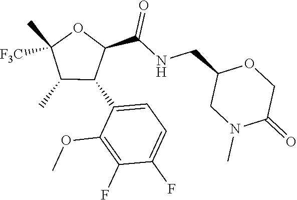 [SMILES: COc1c([C@H]2[C@H](C(=O)NC[C@@H]3CN(C)C(=O)CO3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(((R)-4-methyl-5-oxomorpholin-2-yl)methyl)-5-
(trifluoromethyl)tetrahydrofuran-2-carboxamide

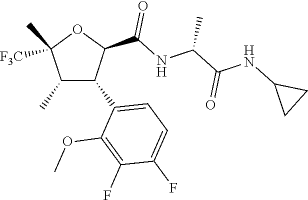

(2R,3S,4S,5R)-N-((R)-1-(cyclopropylamino)-1-oxopropan-2-
yl)-3-(3,4-difluoro-2-methoxyphenyl)-4,5-dimethyl-5-
(trifluoromethyl)tetrahydrofuran-2-carboxamide

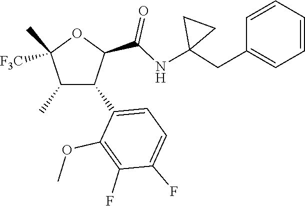 [SMILES: COc1c([C@H]2[C@H](C(=O)NC3(Cc4ccccc4)CC3)O[C@@](C)(C(F)(F)F)[C@H]2C)ccc(F)c1F]

(2R,3S,4S,5R)-N-(1-benzylcyclopropyl)-3-(3,4-
difluoro-2-methoxyphenyl)-4,5-dimethyl-5-
(trifluoromethyl)tetrahydrofuran-2-carboxamide

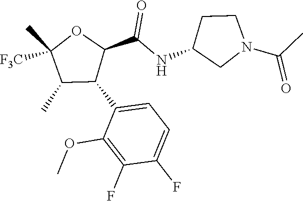 [SMILES: COc1c([C@H]2[C@H](C(=O)N[C@@H]3CCN(C(C)=O)C3)O[C@@](C)(C(F)(F)F)[C@H]2C)ccc(F)c1F]

(2R,3S,4S,5R)-N-((R)-1-acetylpyrrolidin-3-yl)-3-
(3,4-difluoro-2-methoxyphenyl)-4,5-dimethyl-5-
(trifluoromethyl)tetrahydrofuran-2-carboxamide

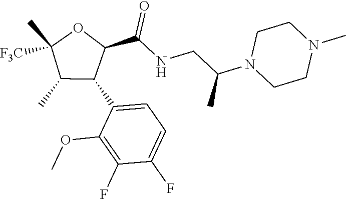 [SMILES: COc1c([C@H]2[C@H](C(=O)NC[C@H](C)N3CCN(C)CC3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-((S)-2-(4-methylpiperazin-1-
yl)propyl)-5-(trifluoromethyl)tetrahydrofuran-2-
carboxamide

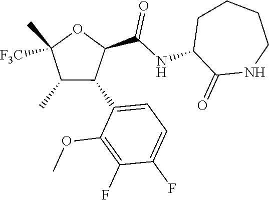 [SMILES: COc1c([C@H]2[C@H](C(=O)N[C@@H]3CCCCNC3=O)O[C@@](C)(C(F)(F)F)[C@H]2C)ccc(F)c1F]

(2R,3S,4S,5R)-3-(3,4-difluoro-2-methoxyphenyl)-4,5-
dimethyl-N-((R)-2-oxoazepan-3-yl)-5-
(trifluoromethyl)tetrahydrofuran-2-carboxamide

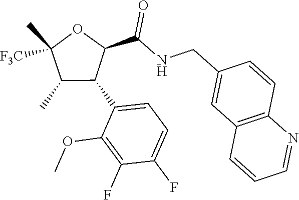 [SMILES: COc1c([C@H]2[C@H](C(=O)NCc3ccc4ncccc4c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(quinolin-6-ylmethyl)-5-
(trifluoromethyl)tetrahydrofuran-2-carboxamide

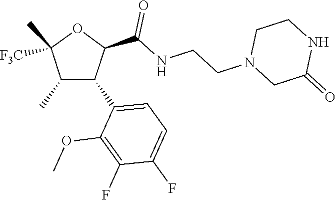 [SMILES: COc1c([C@H]2[C@H](C(=O)NCCN3CCNC(=O)C3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(3-oxopiperazin-1-yl)ethyl)-5-
(trifluoromethyl)tetrahydrofuran-2-carboxamide

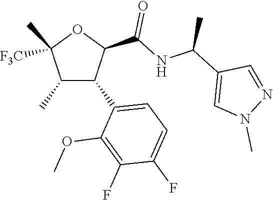 [SMILES: COc1c([C@H]2[C@H](C(=O)N[C@@H](C)c3cnn(C)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-((S)-1-(1-methyl-1H-pyrazol-4-yl)ethyl)-5-
(trifluoromethyl)tetrahydrofuran-2-carboxamide

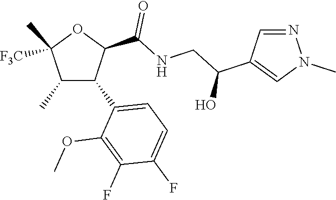 [SMILES: COc1c([C@H]2[C@H](C(=O)NC[C@H](O)c3cnn(C)c3)O[C@@](C)(C(F)(F)F)[C@H]2C)ccc(F)c1F]

(2R,3S,4S,5R)-3-(3,4-difluoro-2-methoxyphenyl)-N-((R)-
2-hydroxy-2-(1-methyl-1H-pyrazol-4-yl)ethyl)-4,5-
dimethyl-5-(trifluoromethyl)tetrahydrofuran-2-carboxamide

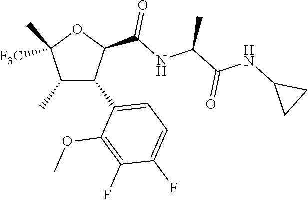 [SMILES: COc1c([C@H]2[C@H](C(=O)N[C@@H](C)C(=O)NC3CC3)O[C@@](C)(C(F)(F)F)[C@H]2C)ccc(F)c1F]

(2R,3S,4S,5R)-N-((S)-1-(cyclopropylamino)-1-oxopropan-2-
yl)-3-(3,4-difluoro-2-methoxyphenyl)-4,5-dimethyl-5-
(trifluoromethyl)tetrahydrofuran-2-carboxamide

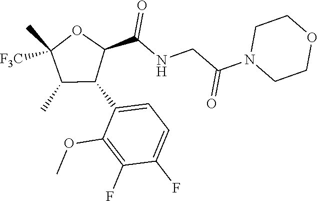 [SMILES: COc1c([C@H]2[C@H](C(=O)NCC(=O)N3CCOCC3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-morpholino-2-oxoethyl)-5-
(trifluoromethyl)tetrahydrofuran-2-carboxamide

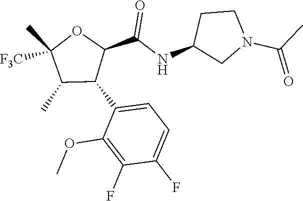 [SMILES: COc1c([C@H]2[C@H](C(=O)N[C@H]3CCN(C(C)=O)C3)O[C@@](C)(C(F)(F)F)[C@H]2C)ccc(F)c1F]

(2R,3S,4S,5R)-N-((S)-1-acetylpyrrolidin-3-yl)-3-(3,4-difluoro-2-
methoxyphenyl)-4,5-dimethyl-5-(trifluoromethyl)tetrahydrofuran-2-
carboxamide

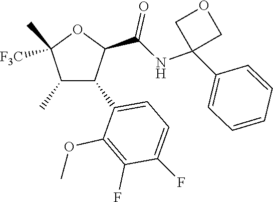 [SMILES: COc1c([C@H]2[C@H](C(=O)NC3(c4ccccc4)COC3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(3-phenyloxetan-3-yl)-5-
(trifluoromethyl)tetrahydrofuran-2-carboxamide

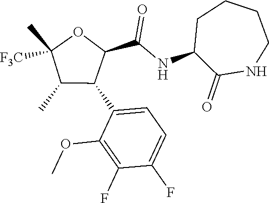 [SMILES: COc1c([C@H]2[C@H](C(=O)N[C@H]3CCCCNC3=O)O[C@@](C)(C(F)(F)F)[C@H]2C)ccc(F)c1F]

(2R,3S,4S,5R)-3-(3,4-difluoro-2-methoxyphenyl)-4,5-
dimethyl-N-((S)-2-oxoazepan-3-yl)-5-
(trifluoromethyl)tetrahydrofuran-2-carboxamide

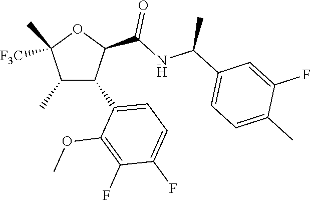 [SMILES: COc1c([C@H]2[C@H](C(=O)N[C@@H](C)c3ccc(C)c(F)c3)O[C@@](C)(C(F)(F)F)[C@H]2C)ccc(F)c1F]

(2R,3S,4S,5R)-3-(3,4-difluoro-2-methoxyphenyl)-N-((S)-
1-(3-fluoro-4-methylphenyl)ethyl)-4,5-dimethyl-5-
(trifluoromethyl)tetrahydrofuran-2-carboxamide

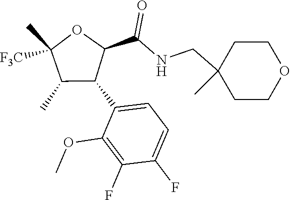 [SMILES: COc1c([C@H]2[C@H](C(=O)NCC3(C)CCOCC3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
((4-methyltetrahydro-2H-pyran-4-yl)methyl)-5-
(trifluoromethyl)tetrahydrofuran-2-carboxamide

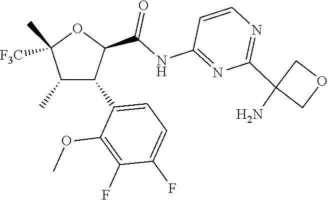 [SMILES: COc1c([C@H]2[C@H](C(=O)Nc3ccnc(C4(N)COC4)n3)O[C@@](C)(C(F)(F)F)[C@H]2C)ccc(F)c1F]

(2R,3S,4S,5R)-N-(2-(3-aminooxetan-3-yl)pyrimidin-4-yl)-
3-(3,4-difluoro-2-methoxyphenyl)-4,5-dimethyl-5-
(trifluoromethyl)tetrahydrofuran-2-carboxamide

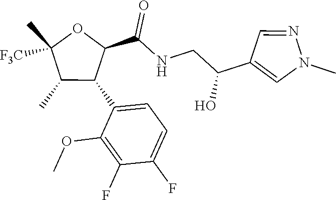 [SMILES: COc1c([C@H]2[C@H](C(=O)NC[C@@H](O)c3cnn(C)c3)O[C@@](C)(C(F)(F)F)[C@H]2C)ccc(F)c1F]

(2R,3S,4S,5R)-3-(3,4-difluoro-2-methoxyphenyl)-N-((S)-2-
hydroxy-2-(1-methyl-1H-pyrazol-4-yl)ethyl)-4,5-dimethyl-
5-(trifluoromethyl)tetrahydrofuran-2-carboxamide

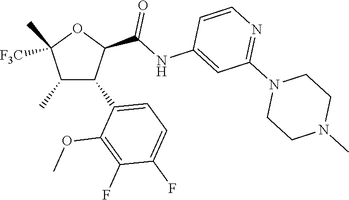 [SMILES: COc1c([C@H]2[C@H](C(=O)Nc3ccnc(N4CCN(C)CC4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(4-methylpiperazin-1-yl)pyridin-
4-yl)-5-(trifluoromethyl)tetrahydrofuran-2-
carboxamide

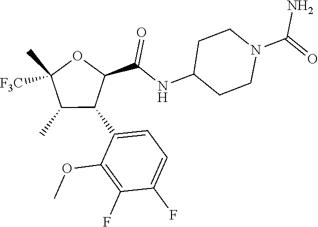 [SMILES: COc1c([C@H]2[C@H](C(=O)NC3CCN(C(N)=O)CC3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)piperidine-1-
carboxamide

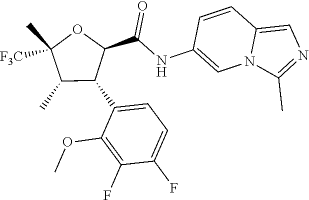 [SMILES: COc1c([C@H]2[C@H](C(=O)Nc3ccc4cnc(C)n4c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(3-methylimidazo[1,5-a]pyridin-6-
yl)-5-(trifluoromethyl)tetrahydrofuran-2-
carboxamide

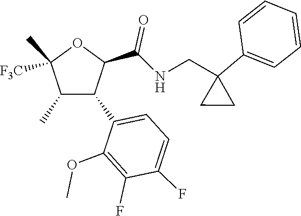 [SMILES: COc1c([C@H]2[C@H](C(=O)NCC3(c4ccccc4)CC3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-((1-phenylcyclopropyl)methyl)-5-
(trifluoromethyl)tetrahydrofuran-2-carboxamide

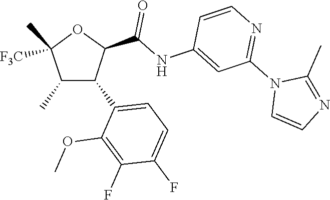 [SMILES: COc1c([C@H]2[C@H](C(=O)Nc3ccnc(-n4ccnc4C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-(2-methyl-1H-imidazol-1-
yl)pyridin-4-yl)-5-(trifluoromethyl)tetrahydrofuran-
2-carboxamide

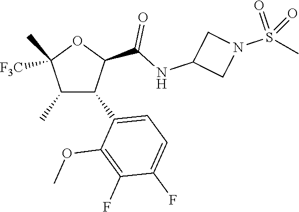 [SMILES: COc1c([C@H]2[C@H](C(=O)NC3CN(S(C)(=O)=O)C3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(1-(methylsulfonyl)azetidin-3-yl)-5-
(trifluoromethyl)tetrahydrofuran-2-carboxamide

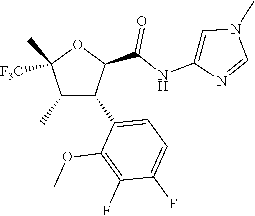 [SMILES: COc1c([C@H]2[C@H](C(=O)Nc3cn(C)cn3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(1-methyl-1H-imidazol-4-yl)-5-
(trifluoromethyl)tetrahydrofuran-2-carboxamide

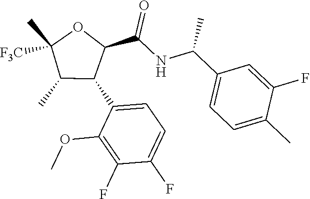 [SMILES: COc1c([C@H]2[C@H](C(=O)N[C@H](C)c3ccc(C)c(F)c3)O[C@@](C)(C(F)(F)F)[C@H]2C)ccc(F)c1F]

(2R,3S,4S,5R)-3-(3,4-difluoro-2-methoxyphenyl)-N-
((R)-1-(3-fluoro-4-methylphenyl)ethyl)-4,5-dimethyl-5-
(trifluoromethyl)tetrahydrofuran-2-carboxamide

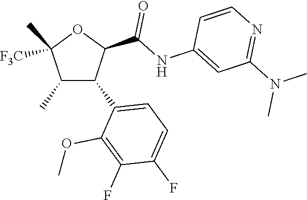 [SMILES: COc1c([C@H]2[C@H](C(=O)Nc3ccnc(N(C)C)c3)O[C@@](C)(C(F)(F)F)[C@H]2C)ccc(F)c1F]

(2R,3S,4S,5R)-3-(3,4-difluoro-2-methoxyphenyl)-
N-(2-(dimethylamino)pyridin-4-yl)-4,5-dimethyl-5-
(trifluoromethyl)tetrahydrofuran-2-carboxamide

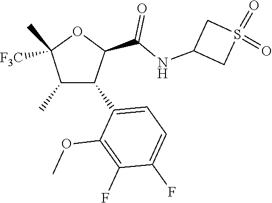 [SMILES: COc1c([C@H]2[C@H](C(=O)NC3CS(=O)(=O)C3)O[C@@](C)(C(F)(F)F)[C@H]2C)ccc(F)c1F]

(2R,3S,4S,5R)-3-(3,4-difluoro-2-methoxyphenyl)-N-(1,1-
dioxidothietan-3-yl)-4,5-dimethyl-5-
(trifluoromethyl)tetrahydrofuran-2-carboxamide

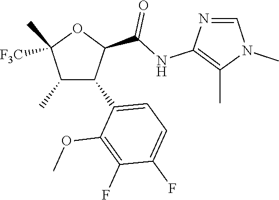 [SMILES: COc1c([C@H]2[C@H](C(=O)Nc3ncn(C)c3C)O[C@@](C)(C(F)(F)F)[C@H]2C)ccc(F)c1F]

(2R,3S,4S,5R)-3-(3,4-difluoro-2-methoxyphenyl)-
N-(1,5-dimethyl-1H-imidazol-4-yl)-4,5-dimethyl-5-
(trifluoromethyl)tetrahydrofuran-2-carboxamide

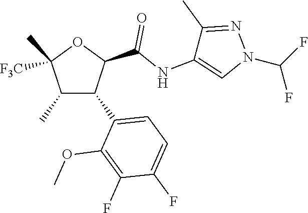 [SMILES: COc1c([C@H]2[C@H](C(=O)Nc3cn(C(F)F)nc3C)O[C@@](C)(C(F)(F)F)[C@H]2C)ccc(F)c1F]

(2R,3S,4S,5R)-3-(3,4-difluoro-2-
methoxyphenyl)-N-(1-(difluoromethyl)-3-methyl-
1H-pyrazol-4-yl)-4,5-dimethyl-5-
(trifluoromethyl)tetrahydrofuran-2-carboxamide

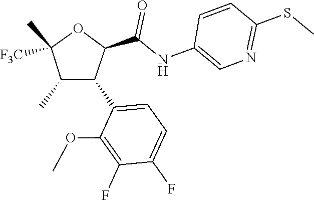 [SMILES: COc1c([C@H]2[C@H](C(=O)Nc3ccc(SC)nc3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(6-(methylthio)pyridin-3-yl)-5-
(trifluoromethyl)tetrahydrofuran-2-carboxamide

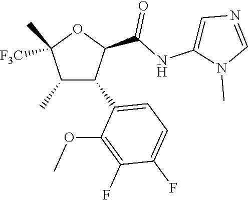 [SMILES: COc1c([C@H]2[C@H](C(=O)Nc3cncn3C)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(1-methyl-1H-imidazol-5-yl)-5-
(trifluoromethyl)tetrahydrofuran-2-carboxamide

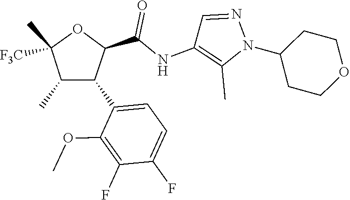 [SMILES: COc1c([C@H]2[C@H](C(=O)Nc3cnn(C4CCOCC4)c3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(5-methyl-1-
(tetrahydro-2H-pyran-4-yl)-1H-pyrazol-4-yl)-5-
(trifluoromethyl)tetrahydrofuran-2-carboxamide

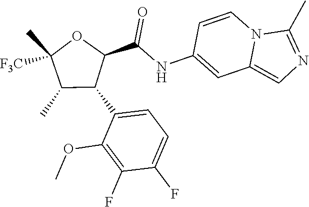 [SMILES: COc1c([C@H]2[C@H](C(=O)Nc3ccn4c(C)ncc4c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(3-methylimidazo[1,5-a]pyridin-7-
yl)-5-(trifluoromethyl)tetrahydrofuran-2-
carboxamide

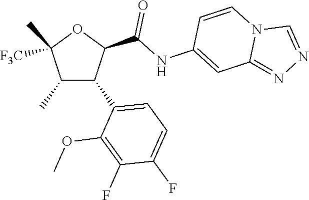 [SMILES: COc1c([C@H]2[C@H](C(=O)Nc3ccn4cnnc4c3)O[C@@](C)(C(F)(F)F)[C@H]2C)ccc(F)c1F]

(2R,3S,4S,5R)-N-([1,2,4]triazolo[4,3-a]pyridin-7-
yl)-3-(3,4-difluoro-2-methoxyphenyl)-4,5-dimethyl-
5-(trifluoromethyl)tetrahydrofuran-2-carboxamide

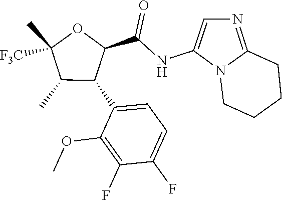 [SMILES: COc1c([C@H]2[C@H](C(=O)Nc3cnc4n3CCCC4)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(5,6,7,8-tetrahydroimidazo[1,2-
a]pyridin-3-yl)-5-(trifluoromethyl)tetrahydrofuran-
2-carboxamide

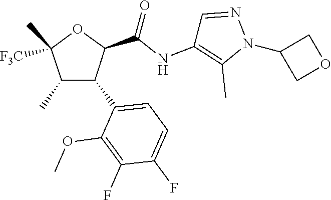 [SMILES: COc1c([C@H]2[C@H](C(=O)Nc3cnn(C4COC4)c3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(5-methyl-1-
(oxetan-3-yl)-1H-pyrazol-4-yl)-5-
(trifluoromethyl)tetrahydrofuran-2-carboxamide

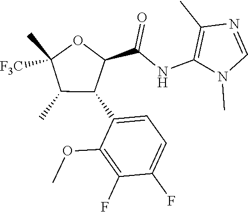 [SMILES: COc1c([C@H]2[C@H](C(=O)Nc3c(C)ncn3C)O[C@@](C)(C(F)(F)F)[C@H]2C)ccc(F)c1F]

(2R,3S,4S,5R)-3-(3,4-difluoro-2-methoxyphenyl)-
N-(1,4-dimethyl-1H-imidazol-5-yl)-4,5-dimethyl-5-
(trifluoromethyl)tetrahydrofuran-2-carboxamide

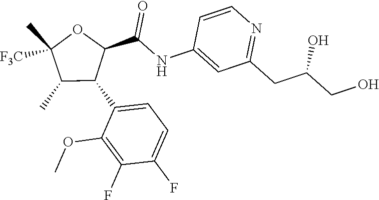 [SMILES: COc1c([C@H]2[C@H](C(=O)Nc3ccnc(C[C@H](O)CO)c3)O[C@@](C)(C(F)(F)F)[C@H]2C)ccc(F)c1F]

(2R,3S,4S,5R)-3-(3,4-difluoro-2-methoxyphenyl)-
N-(2-((S)-2,3-dihydroxypropyl)pyridin-4-yl)-4,5-
dimethyl-5-(trifluoromethyl)tetrahydrofuran-2-
carboxamide

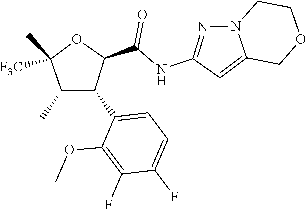 [SMILES: COc1c([C@H]2[C@H](C(=O)Nc3cc4n(n3)CCOC4)O[C@@](C)(C(F)(F)F)[C@H]2C)ccc(F)c1F]

(2R,3S,4S,5R)-3-(3,4-difluoro-2-methoxyphenyl)-
N-(6,7-dihydro-4H-pyrazolo[5,1-c][1,4]oxazin-2-
yl)-4,5-dimethyl-5-
(trifluoromethyl)tetrahydrofuran-2-carboxamide

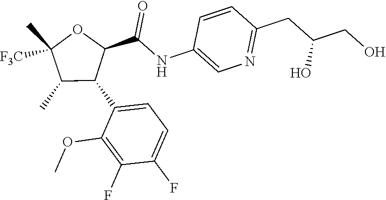 [SMILES: COc1c([C@H]2[C@H](C(=O)Nc3ccc(C[C@@H](O)CO)nc3)O[C@@](C)(C(F)(F)F)[C@H]2C)ccc(F)c1F]

(2R,3S,4S,5R)-3-(3,4-difluoro-2-methoxyphenyl)-
N-(6-((R)-2,3-dihydroxypropyl)pyridin-3-yl)-4,5-
dimethyl-5-(trifluoromethyl)tetrahydrofuran-2-
carboxamide

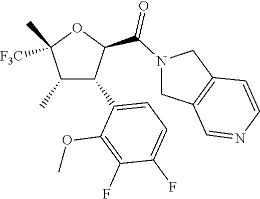 [SMILES: COc1c([C@H]2[C@H](C(=O)N3Cc4ccncc4C3)O[C@@](C)(C(F)(F)F)[C@H]2C)ccc(F)c1F]

((2R,3S,4S,5R)-3-(3,4-difluoro-2-
methoxyphenyl)-4,5-dimethyl-5-
(trifluoromethyl)tetrahydrofuran-2-yl)(1,3-
dihydro-2H-pyrrolo[3,4-c]pyridin-2-yl)methanone

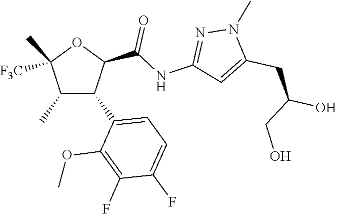 [SMILES: COc1c([C@H]2[C@H](C(=O)Nc3cc(C[C@@H](O)CO)n(C)n3)O[C@@](C)(C(F)(F)F)[C@H]2C)ccc(F)c1F]

(2R,3S,4S,5R)-3-(3,4-difluoro-2-methoxyphenyl)-
N-(5-((R)-2,3-dihydroxypropyl)-1-methyl-1H-
pyrazol-3-yl)-4,5-dimethyl-5-
(trifluoromethyl)tetrahydrofuran-2-carboxamide

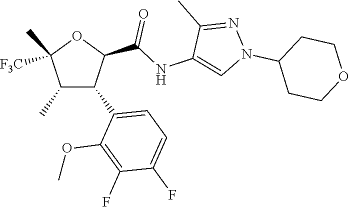 [SMILES: COc1c([C@H]2[C@H](C(=O)Nc3cn(C4CCOCC4)nc3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(3-methyl-1-
(tetrahydro-2H-pyran-4-yl)-1H-pyrazol-4-yl)-5-
(trifluoromethyl)tetrahydrofuran-2-carboxamide

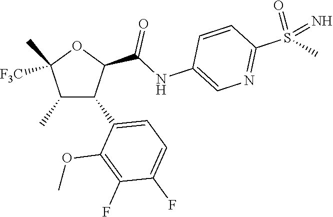 [SMILES: COc1c([C@H]2[C@H](C(=O)Nc3ccc([S@](C)(=N)=O)nc3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(6-(S-methylsulfonimidoyl)pyridin-
3-yl)-5-(trifluoromethyl)tetrahydrofuran-2-
carboxamide

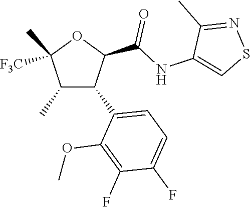 [SMILES: COc1c([C@H]2[C@H](C(=O)Nc3csnc3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(3-
methylisothiazol-4-yl)-5-
(trifluoromethyl)tetrahydrofuran-2-carboxamide

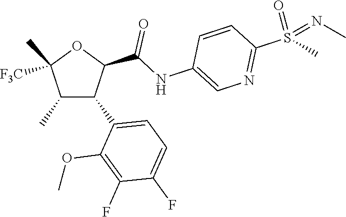 [SMILES: CN=[S@](C)(=O)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-
N-(6-(N,S-dimethylsulfonimidoyl)pyridin-3-yl)-4,5-
dimethyl-5-(trifluoromethyl)tetrahydrofuran-2-
carboxamide

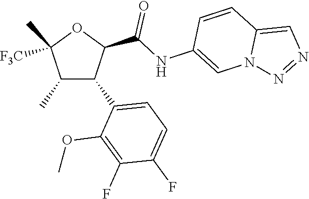 [SMILES: COc1c([C@H]2[C@H](C(=O)Nc3ccc4cnnn4c3)O[C@@](C)(C(F)(F)F)[C@H]2C)ccc(F)c1F]

(2R,3S,4S,5R)-N-([1,2,3]triazolo[1,5-a]pyridin-6-
yl)-3-(3,4-difluoro-2-methoxyphenyl)-4,5-dimethyl-
5-(trifluoromethyl)tetrahydrofuran-2-carboxamide

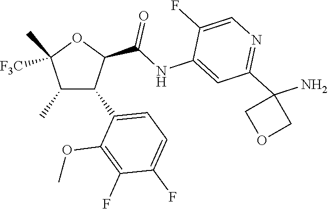 [SMILES: COc1c([C@H]2[C@H](C(=O)Nc3cc(C4(N)COC4)ncc3F)O[C@@](C)(C(F)(F)F)[C@H]2C)ccc(F)c1F]

(2R,3S,4S,5R)-N-(2-(3-aminooxetan-3-yl)-5-
fluoropyridin-4-yl)-3-(3,4-difluoro-2-
methoxyphenyl)-4,5-dimethyl-5-
(trifluoromethyl)tetrahydrofuran-2-carboxamide

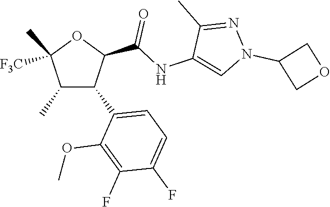 [SMILES: COc1c([C@H]2[C@H](C(=O)Nc3cn(C4COC4)nc3C)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(3-methyl-1-
(oxetan-3-yl)-1H-pyrazol-4-yl)-5-
(trifluoromethyl)tetrahydrofuran-2-carboxamide

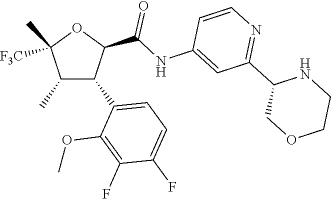 [SMILES: COc1c([C@H]2[C@H](C(=O)Nc3ccnc([C@H]4COCCN4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((S)-morpholin-3-yl)pyridin-4-
yl)-5-(trifluoromethyl)tetrahydrofuran-2-
carboxamide

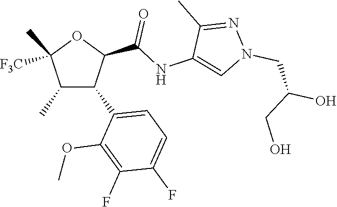 [SMILES: COc1c([C@H]2[C@H](C(=O)Nc3cn(C[C@H](O)CO)nc3C)O[C@@](C)(C(F)(F)F)[C@H]2C)ccc(F)c1F]

(2R,3S,4S,5R)-3-(3,4-difluoro-2-methoxyphenyl)-
N-(1-((S)-2,3-dihydroxypropyl)-3-methyl-1H-
pyrazol-4-yl)-4,5-dimethyl-5-
(trifluoromethyl)tetrahydrofuran-2-carboxamide

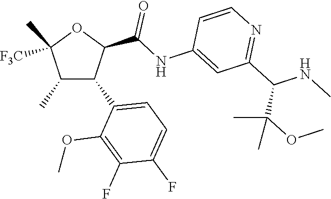 [SMILES: CN[C@@H](c1cc(NC(=O)[C@@H]2O[C@@](C)(C(F)(F)F)[C@@H](C)[C@H]2c2ccc(F)c(F)c2OC)ccn1)C(C)(C)OC]

(2R,3S,4S,5R)-3-(3,4-difluoro-2-methoxyphenyl)-
N-(2-((S)-2-methoxy-2-methyl-1-
(methylamino)propyl)pyridin-4-yl)-4,5-dimethyl-5-
(trifluoromethyl)tetrahydrofuran-2-carboxamide

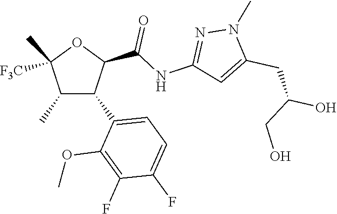 [SMILES: COc1c([C@H]2[C@H](C(=O)Nc3cc(C[C@H](O)CO)n(C)n3)O[C@@](C)(C(F)(F)F)[C@H]2C)ccc(F)c1F]

(2R,3S,4S,5R)-3-(3,4-difluoro-2-methoxyphenyl)-
N-(5-((S)-2,3-dihydroxypropyl)-1-methyl-1H-
pyrazol-3-yl)-4,5-dimethyl-5-
(trifluoromethyl)tetrahydrofuran-2-carboxamide

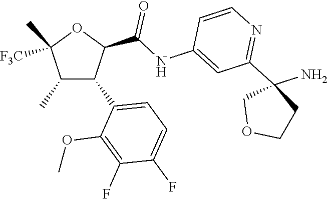 [SMILES: COc1c([C@H]2[C@H](C(=O)Nc3ccnc([C@@]4(N)CCOC4)c3)O[C@@](C)(C(F)(F)F)[C@H]2C)ccc(F)c1F]

(2R,3S,4S,5R)-N-(2-((S)-3-aminotetrahydrofuran-
3-yl)pyridin-4-yl)-3-(3,4-difluoro-2-
methoxyphenyl)-4,5-dimethyl-5-
(trifluoromethyl)tetrahydrofuran-2-carboxamide

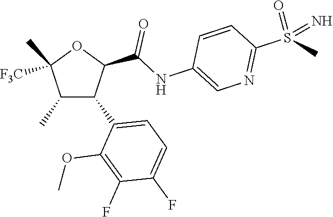 [SMILES: COc1c([C@H]2[C@H](C(=O)Nc3ccc([S@@](C)(=N)=O)nc3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(6-(S-methylsulfonimidoyl)pyridin-
3-yl)-5-(trifluoromethyl)tetrahydrofuran-2-
carboxamide

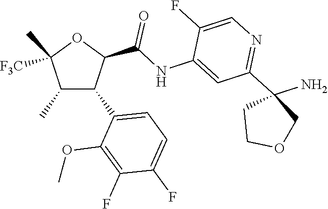 [SMILES: COc1c([C@H]2[C@H](C(=O)Nc3cc([C@]4(N)CCOC4)ncc3F)O[C@@](C)(C(F)(F)F)[C@H]2C)ccc(F)c1F]

(2R,3S,4S,5R)-N-(2-((R)-3-aminotetrahydrofuran-
3-yl)-5-fluoropyridin-4-yl)-3-(3,4-difluoro-2-
methoxyphenyl)-4,5-dimethyl-5-
(trifluoromethyl)tetrahydrofuran-2-carboxamide

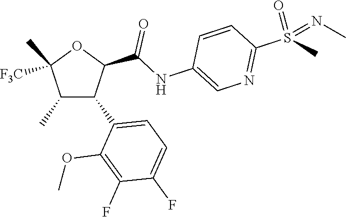 [SMILES: CN=[S@@](C)(=O)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-
N-(6-(N,S-dimethylsulfonimidoyl)pyridin-3-yl)-4,5-
dimethyl-5-(trifluoromethyl)tetrahydrofuran-2-
carboxamide

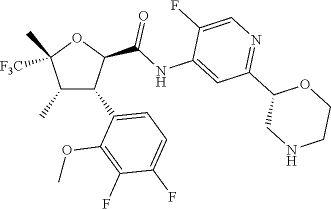 [SMILES: COc1c([C@H]2[C@H](C(=O)Nc3cc([C@H]4CNCCO4)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((R)-morpholin-2-yl)pyridin-4-yl)-4,5-
dimethyl-5-(trifluoromethyl)tetrahydrofuran-2-
carboxamide

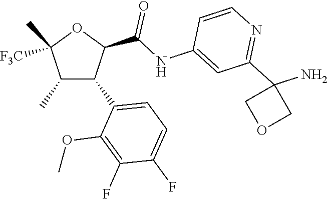 [SMILES: COc1c([C@H]2[C@H](C(=O)Nc3ccnc(C4(N)COC4)c3)O[C@@](C)(C(F)(F)F)[C@H]2C)ccc(F)c1F]

(2R,3S,4S,5R)-N-(2-(3-aminooxetan-3-yl)pyridin-
4-yl)-3-(3,4-difluoro-2-methoxyphenyl)-4,5-
dimethyl-5-(trifluoromethyl)tetrahydrofuran-2-
carboxamide

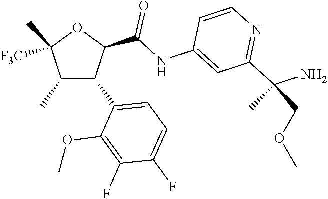 [SMILES: COC[C@](C)(N)c1cc(NC(=O)[C@@H]2O[C@@](C)(C(F)(F)F)[C@@H](C)[C@H]2c2ccc(F)c(F)c2OC)ccn1]

(2R,3S,4S,5R)-N-(2-((R)-2-amino-1-
methoxypropan-2-yl)pyridin-4-yl)-3-(3,4-difluoro-
2-methoxyphenyl)-4,5-dimethyl-5-
(trifluoromethyl)tetrahydrofuran-2-carboxamide

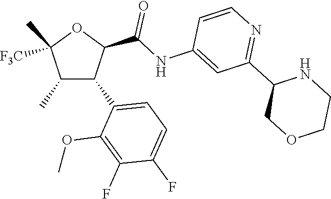 [SMILES: COc1c([C@H]2[C@H](C(=O)Nc3ccnc([C@@H]4COCCN4)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((R)-morpholin-3-yl)pyridin-4-
yl)-5-(trifluoromethyl)tetrahydrofuran-2-
carboxamide

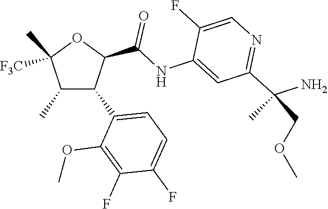 [SMILES: COC[C@](C)(N)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-N-(2-((R)-2-amino-1-
methoxypropan-2-yl)-5-fluoropyridin-4-yl)-3-(3,4-
difluoro-2-methoxyphenyl)-4,5-dimethyl-5-
(trifluoromethyl)tetrahydrofuran-2-carboxamide

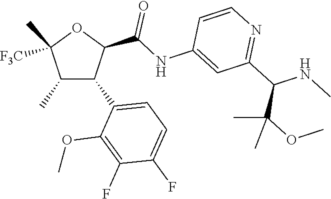 [SMILES: CN[C@H](c1cc(NC(=O)[C@@H]2O[C@@](C)(C(F)(F)F)[C@@H](C)[C@H]2c2ccc(F)c(F)c2OC)ccn1)C(C)(C)OC]

(2R,3S,4S,5R)-3-(3,4-difluoro-2-methoxyphenyl)-
N-(2-((R)-2-methoxy-2-methyl-1-
(methylamino)propyl)pyridin-4-yl)-4,5-dimethyl-5-
(trifluoromethyl)tetrahydrofuran-2-carboxamide

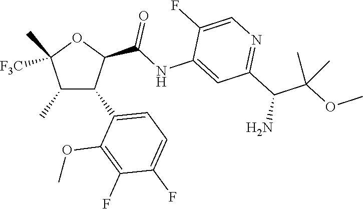 [SMILES: COc1c([C@H]2[C@H](C(=O)Nc3cc([C@@H](N)C(C)(C)OC)ncc3F)O[C@@](C)(C(F)(F)F)[C@H]2C)ccc(F)c1F]

(2R,3S,4S,5R)-N-(2-((R)-1-amino-2-methoxy-2-
methylpropyl)-5-fluoropyridin-4-yl)-3-(3,4-difluoro-
2-methoxyphenyl)-4,5-dimethyl-5-
(trifluoromethyl)tetrahydrofuran-2-carboxamide

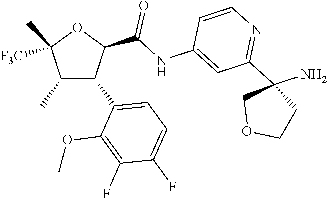 [SMILES: COc1c([C@H]2[C@H](C(=O)Nc3ccnc([C@]4(N)CCOC4)c3)O[C@@](C)(C(F)(F)F)[C@H]2C)ccc(F)c1F]

(2R,3S,4S,5R)-N-(2-((R)-3-aminotetrahydrofuran-
3-yl)pyridin-4-yl)-3-(3,4-difluoro-2-
methoxyphenyl)-4,5-dimethyl-5-
(trifluoromethyl)tetrahydrofuran-2-carboxamide

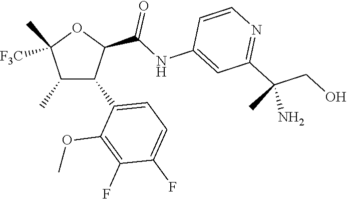 [SMILES: COc1c([C@H]2[C@H](C(=O)Nc3ccnc([C@@](C)(N)CO)c3)O[C@@](C)(C(F)(F)F)[C@H]2C)ccc(F)c1F]

(2R,3S,4S,5R)-N-(2-((R)-2-amino-1-
hydroxypropan-2-yl)pyridin-4-yl)-3-(3,4-difluoro-2-
methoxyphenyl)-4,5-dimethyl-5-
(trifluoromethyl)tetrahydrofuran-2-carboxamide

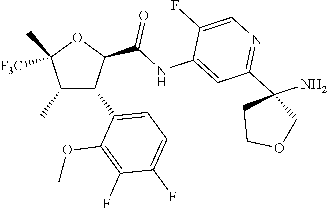 [SMILES: COc1c([C@H]2[C@H](C(=O)Nc3cc([C@@]4(N)CCOC4)ncc3F)O[C@@](C)(C(F)(F)F)[C@H]2C)ccc(F)c1F]

(2R,3S,4S,5R)-N-(2-((S)-3-aminotetrahydrofuran-
3-yl)-5-fluoropyridin-4-yl)-3-(3,4-difluoro-2-
methoxyphenyl)-4,5-dimethyl-5-
(trifluoromethyl)tetrahydrofuran-2-carboxamide

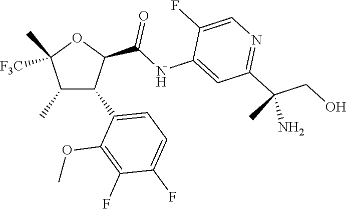 [SMILES: COc1c([C@H]2[C@H](C(=O)Nc3cc([C@@](C)(N)CO)ncc3F)O[C@@](C)(C(F)(F)F)[C@H]2C)ccc(F)c1F]

(2R,3S,4S,5R)-N-(2-((R)-2-amino-1-
hydroxypropan-2-yl)-5-fluoropyridin-4-yl)-3-(3,4-
difluoro-2-methoxyphenyl)-4,5-dimethyl-5-
(trifluoromethyl)tetrahydrofuran-2-carboxamide

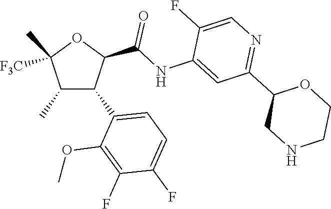 [SMILES: COc1c([C@H]2[C@H](C(=O)Nc3cc([C@@H]4CNCCO4)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((S)-morpholin-2-yl)pyridin-4-yl)-4,5-
dimethyl-5-(trifluoromethyl)tetrahydrofuran-2-
carboxamide

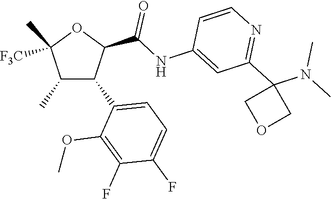 [SMILES: COc1c([C@H]2[C@H](C(=O)Nc3ccnc(C4(N(C)C)COC4)c3)O[C@@](C)(C(F)(F)F)[C@H]2C)ccc(F)c1F]

(2R,3S,4S,5R)-3-(3,4-difluoro-2-methoxyphenyl)-
N-(2-(3-(dimethylamino)oxetan-3-yl)pyridin-4-yl)-
4,5-dimethyl-5-(trifluoromethyl)tetrahydrofuran-2-
carboxamide

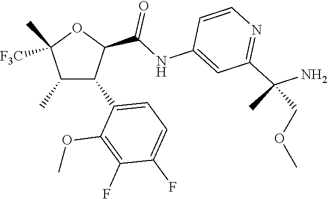 [SMILES: COC[C@@](C)(N)c1cc(NC(=O)[C@@H]2O[C@@](C)(C(F)(F)F)[C@@H](C)[C@H]2c2ccc(F)c(F)c2OC)ccn1]

(2R,3S,4S,5R)-N-(2-((S)-2-amino-1-
methoxypropan-2-yl)pyridin-4-yl)-3-(3,4-difluoro-
2-methoxyphenyl)-4,5-dimethyl-5-
(trifluoromethyl)tetrahydrofuran-2-carboxamide

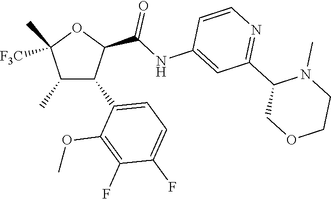 [SMILES: COc1c([C@H]2[C@H](C(=O)Nc3ccnc([C@H]4COCCN4C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((S)-4-methylmorpholin-3-
yl)pyridin-4-yl)-5-(trifluoromethyl)tetrahydrofuran-
2-carboxamide

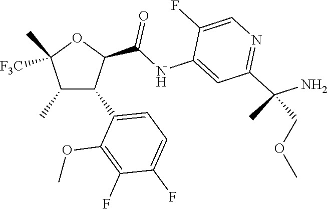 [SMILES: COC[C@@](C)(N)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-N-(2-((S)-2-amino-1-
methoxypropan-2-yl)-5-fluoropyridin-4-yl)-3-(3,4-
difluoro-2-methoxyphenyl)-4,5-dimethyl-5-
(trifluoromethyl)tetrahydrofuran-2-carboxamide

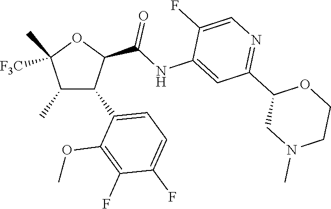 [SMILES: COc1c([C@H]2[C@H](C(=O)Nc3cc([C@H]4CN(C)CCO4)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((R)-4-methylmorpholin-2-yl)pyridin-
4-yl)-4,5-dimethyl-5-
(trifluoromethyl)tetrahydrofuran-2-carboxamide

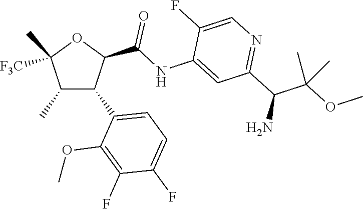 [SMILES: COc1c([C@H]2[C@H](C(=O)Nc3cc([C@H](N)C(C)(C)OC)ncc3F)O[C@@](C)(C(F)(F)F)[C@H]2C)ccc(F)c1F]

(2R,3S,4S,5R)-N-(2-((S)-1-amino-2-methoxy-2-
methylpropyl)-5-fluoropyridin-4-yl)-3-(3,4-difluoro-
2-methoxyphenyl)-4,5-dimethyl-5-
(trifluoromethyl)tetrahydrofuran-2-carboxamide

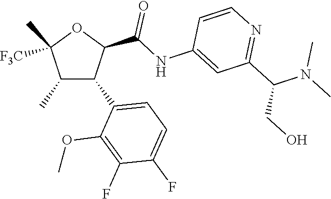 [SMILES: COc1c([C@H]2[C@H](C(=O)Nc3ccnc([C@@H](CO)N(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((S)-1-(dimethylamino)-2-
hydroxyethyl)pyridin-4-yl)-4,5-dimethyl-5-
(trifluoromethyl)tetrahydrofuran-2-carboxamide

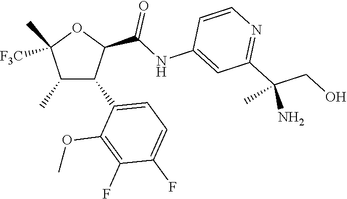 [SMILES: COc1c([C@H]2[C@H](C(=O)Nc3ccnc([C@](C)(N)CO)c3)O[C@@](C)(C(F)(F)F)[C@H]2C)ccc(F)c1F]

(2R,3S,4S,5R)-N-(2-((S)-2-amino-1-
hydroxypropan-2-yl)pyridin-4-yl)-3-(3,4-difluoro-2-
methoxyphenyl)-4,5-dimethyl-5-
(trifluoromethyl)tetrahydrofuran-2-carboxamide

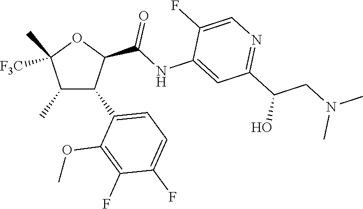 [SMILES: COc1c([C@H]2[C@H](C(=O)Nc3cc([C@@H](O)CN(C)C)ncc3F)O[C@@](C)(C(F)(F)F)[C@H]2C)ccc(F)c1F]

(2R,3S,4S,5R)-3-(3,4-difluoro-2-methoxyphenyl)-
N-(2-((S)-2-(dimethylamino)-1-hydroxyethyl)-5-
fluoropyridin-4-yl)-4,5-dimethyl-5-
(trifluoromethyl)tetrahydrofuran-2-carboxamide

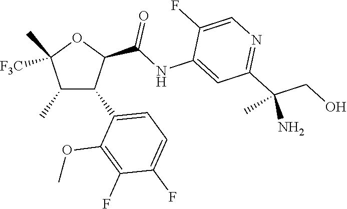 [SMILES: COc1c([C@H]2[C@H](C(=O)Nc3cc([C@](C)(N)CO)ncc3F)O[C@@](C)(C(F)(F)F)[C@H]2C)ccc(F)c1F]

(2R,3S,4S,5R)-N-(2-((S)-2-amino-1-
hydroxypropan-2-yl)-5-fluoropyridin-4-yl)-3-(3,4-
difluoro-2-methoxyphenyl)-4,5-dimethyl-5-
(trifluoromethyl)tetrahydrofuran-2-carboxamide

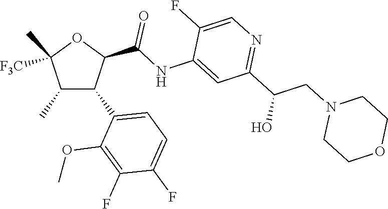 [SMILES: COc1c([C@H]2[C@H](C(=O)Nc3cc([C@@H](O)CN4CCOCC4)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((S)-1-hydroxy-2-
morpholinoethyl)pyridin-4-yl)-4,5-dimethyl-5-
(trifluoromethyl)tetrahydrofuran-2-carboxamide

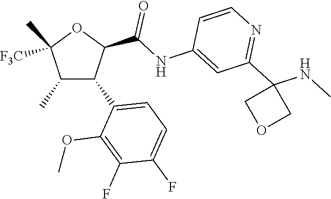 [SMILES: CNC1(c2cc(NC(=O)[C@@H]3O[C@@](C)(C(F)(F)F)[C@@H](C)[C@H]3c3ccc(F)c(F)c3OC)ccn2)COC1]

(2R,3S,4S,5R)-3-(3,4-difluoro-2-methoxyphenyl)-
4,5-dimethyl-N-(2-(3-(methylamino)oxetan-3-
yl)pyridin-4-yl)-5-(trifluoromethyl)tetrahydrofuran-
2-carboxamide

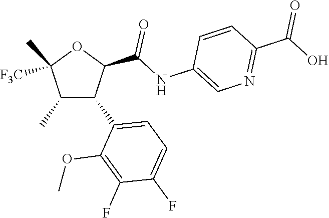 [SMILES: COc1c([C@H]2[C@H](C(=O)Nc3ccc(C(=O)O)nc3)O[C@@](C)(C(F)(F)F)[C@H]2C)ccc(F)c1F]

5-((2R,3S,4S,5R)-3-(3,4-difluoro-2-
methoxyphenyl)-4,5-dimethyl-5-
(trifluoromethyl)tetrahydrofuran-2-
carboxamido)picolinic acid

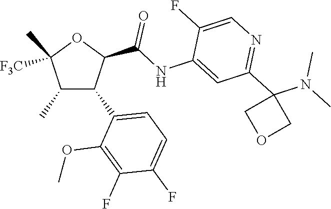 [SMILES: COc1c([C@H]2[C@H](C(=O)Nc3cc(C4(N(C)C)COC4)ncc3F)O[C@@](C)(C(F)(F)F)[C@H]2C)ccc(F)c1F]

(2R,3S,4S,5R)-3-(3,4-difluoro-2-methoxyphenyl)-
N-(2-(3-(dimethylamino)oxetan-3-yl)-5-
fluoropyridin-4-yl)-4,5-dimethyl-5-
(trifluoromethyl)tetrahydrofuran-2-carboxamide

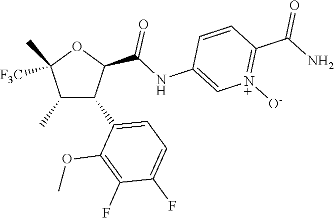 [SMILES: COc1c([C@H]2[C@H](C(=O)Nc3ccc(C(N)=O)[n+]([O-])c3)O[C@@](C)(C(F)(F)F)[C@H]2C)ccc(F)c1F]

2-carbamoyl-5-((2R,3S,4S,5R)-3-(3,4-difluoro-2-
methoxyphenyl)-4,5-dimethyl-5-
(trifluoromethyl)tetrahydrofuran-2-
carboxamido)pyridine 1-oxide

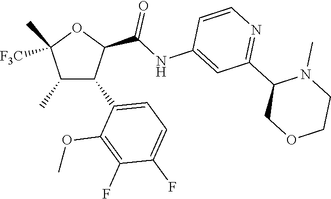 [SMILES: COc1c([C@H]2[C@H](C(=O)Nc3ccnc([C@@H]4COCCN4C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((R)-4-methylmorpholin-3-
yl)pyridin-4-yl)-5-(trifluoromethyl)tetrahydrofuran-
2-carboxamide

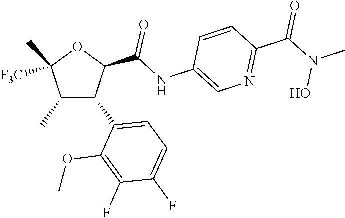 [SMILES: COc1c([C@H]2[C@H](C(=O)Nc3ccc(C(=O)N(C)O)nc3)O[C@@](C)(C(F)(F)F)[C@H]2C)ccc(F)c1F]

5-((2R,3S,4S,5R)-3-(3,4-difluoro-2-
methoxyphenyl)-4,5-dimethyl-5-
(trifluoromethyl)tetrahydrofuran-2-carboxamido)-
N-hydroxy-N-methylpicolinamide

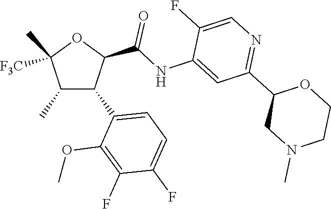 [SMILES: COc1c([C@H]2[C@H](C(=O)Nc3cc([C@@H]4CN(C)CCO4)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((S)-4-methylmorpholin-2-yl)pyridin-
4-yl)-4,5-dimethyl-5-
(trifluoromethyl)tetrahydrofuran-2-carboxamide

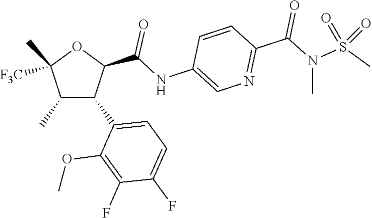 [SMILES: COc1c([C@H]2[C@H](C(=O)Nc3ccc(C(=O)N(C)S(C)(=O)=O)nc3)O[C@@](C)(C(F)(F)F)[C@H]2C)ccc(F)c1F]

5-((2R,3S,4S,5R)-3-(3,4-difluoro-2-
methoxyphenyl)-4,5-dimethyl-5-
(trifluoromethyl)tetrahydrofuran-2-carboxamido)-
N-methyl-N-(methylsulfonyl)picolinamide

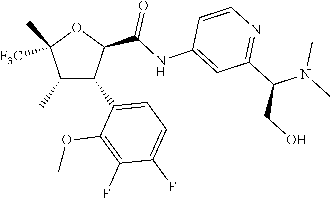 [SMILES: COc1c([C@H]2[C@H](C(=O)Nc3ccnc([C@H](CO)N(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1-(dimethylamino)-2-
hydroxyethyl)pyridin-4-yl)-4,5-dimethyl-5-
(trifluoromethyl)tetrahydrofuran-2-carboxamide

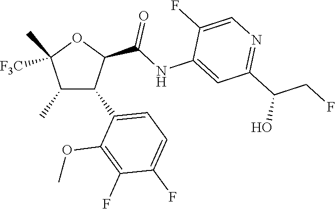 [SMILES: COc1c([C@H]2[C@H](C(=O)Nc3cc([C@@H](O)CF)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((R)-2-fluoro-1-hydroxyethyl)pyridin-
4-yl)-4,5-dimethyl-5-
(trifluoromethyl)tetrahydrofuran-2-carboxamide

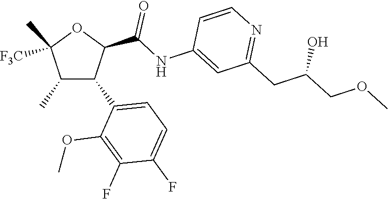 [SMILES: COC[C@@H](O)Cc1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-((S)-2-hydroxy-3-methoxypropyl)pyridin-4-
yl)-4,5-dimethyl-5-(trifluoromethyl)tetrahydrofuran-
2-carboxamide

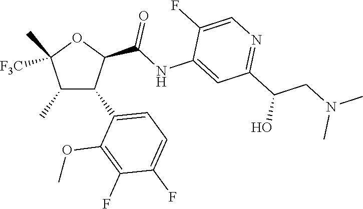 [SMILES: COc1c([C@H]2[C@H](C(=O)Nc3cc([C@@H](O)CN(C)C)ncc3F)O[C@@](C)(C(F)(F)F)[C@H]2C)ccc(F)c1F]

(2R,3S,4S,5R)-3-(3,4-difluoro-2-methoxyphenyl)-
N-(2-((S)-2-(dimethylamino)-1-hydroxyethyl)-5-
fluoropyridin-4-yl)-4,5-dimethyl-5-
(trifluoromethyl)tetrahydrofuran-2-carboxamide

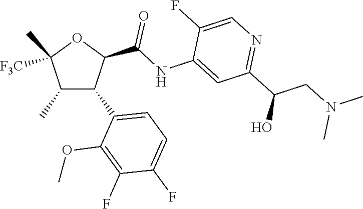 [SMILES: COc1c([C@H]2[C@H](C(=O)Nc3cc([C@H](O)CN(C)C)ncc3F)O[C@@](C)(C(F)(F)F)[C@H]2C)ccc(F)c1F]

(2R,3S,4S,5R)-3-(3,4-difluoro-2-methoxyphenyl)-
N-(2-((R)-2-(dimethylamino)-1-hydroxyethyl)-5-
fluoropyridin-4-yl)-4,5-dimethyl-5-
(trifluoromethyl)tetrahydrofuran-2-carboxamide

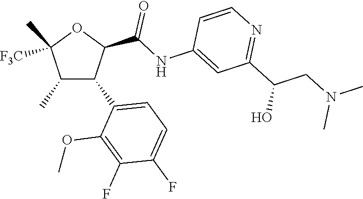 [SMILES: COc1c([C@H]2[C@H](C(=O)Nc3ccnc([C@@H](O)CN(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((S)-2-(dimethylamino)-1-
hydroxyethyl)pyridin-4-yl)-4,5-dimethyl-5-
(trifluoromethyl)tetrahydrofuran-2-carboxamide

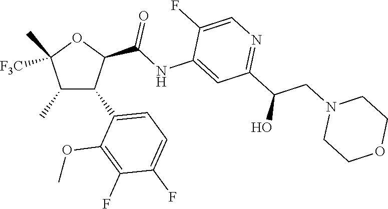 [SMILES: COc1c([C@H]2[C@H](C(=O)Nc3cc([C@H](O)CN4CCOCC4)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((R)-1-hydroxy-2-
morpholinoethyl)pyridin-4-yl)-4,5-dimethyl-5-
(trifluoromethyl)tetrahydrofuran-2-carboxamide

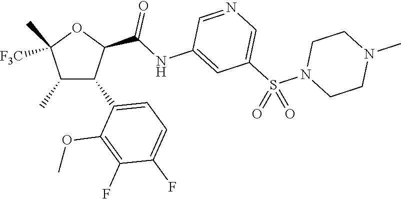 [SMILES: COc1c([C@H]2[C@H](C(=O)Nc3cncc(S(=O)(=O)N4CCN(C)CC4)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(5-((4-
methylpiperazin-1-yl)sulfonyl)pyridin-3-yl)-5-
(trifluoromethyl)tetrahydrofuran-2-carboxamide

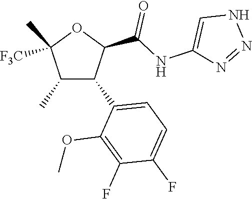 [SMILES: COc1c([C@H]2[C@H](C(=O)Nc3c[nH]nn3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(1H-1,2,3-triazol-4-yl)-5-
(trifluoromethyl)tetrahydrofuran-2-carboxamide

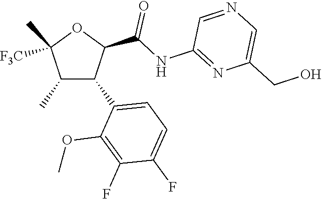 [SMILES: COc1c([C@H]2[C@H](C(=O)Nc3cncc(CO)n3)O[C@@](C)(C(F)(F)F)[C@H]2C)ccc(F)c1F]

(2R,3S,4S,5R)-3-(3,4-difluoro-2-
methoxyphenyl)-N-(6-(hydroxymethyl)pyrazin-
2-yl)-4,5-dimethyl-5-
(trifluoromethyl)tetrahydrofuran-2-carboxamide

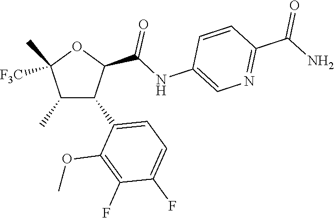 [SMILES: COc1c([C@H]2[C@H](C(=O)Nc3ccc(C(N)=O)nc3)O[C@@](C)(C(F)(F)F)[C@H]2C)ccc(F)c1F]

5-((2R,3S,4S,5R)-3-(3,4-difluoro-2-
methoxyphenyl)-4,5-dimethyl-5-
(trifluoromethyl)tetrahydrofuran-2-
carboxamido)picolinamide

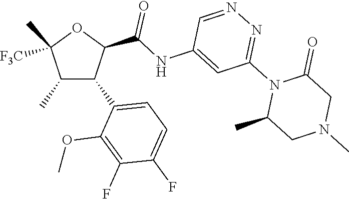 [SMILES: COc1c([C@H]2[C@H](C(=O)Nc3cnnc(N4C(=O)CN(C)C[C@H]4C)c3)O[C@@](C)(C(F)(F)F)[C@H]2C)ccc(F)c1F]

(2R,3S,4S,5R)-3-(3,4-difluoro-2-methoxyphenyl)-
N-(6-((R)-2,4-dimethyl-6-oxopiperazin-1-
yl)pyridazin-4-yl)-4,5-dimethyl-5-
(trifluoromethyl)tetrahydrofuran-2-carboxamide

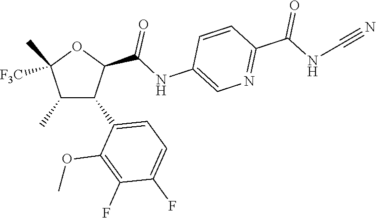 [SMILES: COc1c([C@H]2[C@H](C(=O)Nc3ccc(C(=O)NC#N)nc3)O[C@@](C)(C(F)(F)F)[C@H]2C)ccc(F)c1F]

N-cyano-5-((2R,3S,4S,5R)-3-(3,4-difluoro-2-
methoxyphenyl)-4,5-dimethyl-5-
(trifluoromethyl)tetrahydrofuran-2-
carboxamido)picolinamide

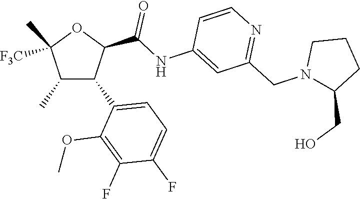 [SMILES: COc1c([C@H]2[C@H](C(=O)Nc3ccnc(CN4CCC[C@H]4CO)c3)O[C@@](C)(C(F)(F)F)[C@H]2C)ccc(F)c1F]

(2R,3S,4S,5R)-3-(3,4-difluoro-2-methoxyphenyl)-
N-(2-(((S)-2-(hydroxymethyl)pyrrolidin-1-
yl)methyl)pyridin-4-yl)-4,5-dimethyl-5-
(trifluoromethyl)tetrahydrofuran-2-carboxamide

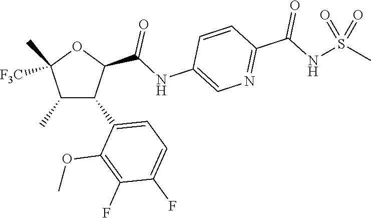 [SMILES: COc1c([C@H]2[C@H](C(=O)Nc3ccc(C(=O)NS(C)(=O)=O)nc3)O[C@@](C)(C(F)(F)F)[C@H]2C)ccc(F)c1F]

5-((2R,3S,4S,5R)-3-(3,4-difluoro-2-
methoxyphenyl)-4,5-dimethyl-5-
(trifluoromethyl)tetrahydrofuran-2-carboxamido)-
N-(methylsulfonyl)picolinamide

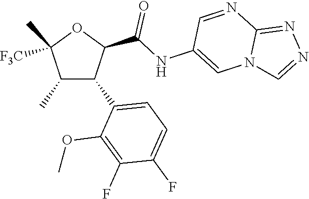 [SMILES: COc1c([C@H]2[C@H](C(=O)Nc3cnc4nncn4c3)O[C@@](C)(C(F)(F)F)[C@H]2C)ccc(F)c1F]

(2R,3S,4S,5R)-N-([1,2,4]triazolo[4,3-
a]pyrimidin-6-yl)-3-(3,4-difluoro-2-
methoxyphenyl)-4,5-dimethyl-5-
(trifluoromethyl)tetrahydrofuran-2-carboxamide

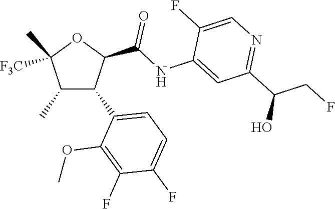 [SMILES: COc1c([C@H]2[C@H](C(=O)Nc3cc([C@H](O)CF)ncc3F)O[C@@](C)(C(F)(F)F)[C@H]2C)ccc(F)c1F]

(2R,3S,4S,5R)-3-(3,4-difluoro-2-methoxyphenyl)-
N-(5-fluoro-2-((S)-2-fluoro-1-hydroxyethyl)pyridin-
4-yl)-4,5-dimethyl-5-
(trifluoromethyl)tetrahydrofuran-2-carboxamide

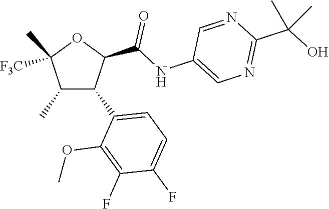 [SMILES: COc1c([C@H]2[C@H](C(=O)Nc3cnc(C(C)(C)O)nc3)O[C@@](C)(C(F)(F)F)[C@H]2C)ccc(F)c1F]

(2R,3S,4S,5R)-3-(3,4-difluoro-2-
methoxyphenyl)-N-(2-(2-hydroxypropan-2-
yl)pyrimidin-5-yl)-4,5-dimethyl-5-
(trifluoromethyl)tetrahydrofuran-2-carboxamide

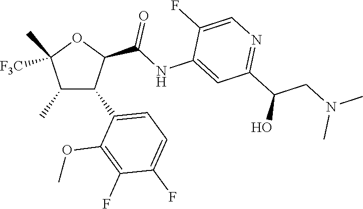 [SMILES: COc1c([C@H]2[C@H](C(=O)Nc3cc([C@H](O)CN(C)C)ncc3F)O[C@@](C)(C(F)(F)F)[C@H]2C)ccc(F)c1F]

(2R,3S,4S,5R)-3-(3,4-difluoro-2-methoxyphenyl)-
N-(2-((R)-2-(dimethylamino)-1-hydroxyethyl)-5-
fluoropyridin-4-yl)-4,5-dimethyl-5-
(trifluoromethyl)tetrahydrofuran-2-carboxamide

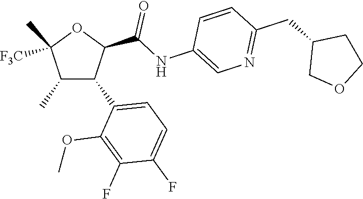 [SMILES: COc1c([C@H]2[C@H](C(=O)Nc3ccc(C[C@@H]4CCOC4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(((R)-
tetrahydrofuran-3-yl)methyl)pyridin-3-yl)-5-
(trifluoromethyl)tetrahydrofuran-2-carboxamide

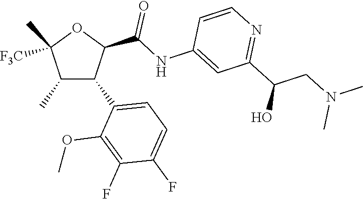 [SMILES: COc1c([C@H]2[C@H](C(=O)Nc3ccnc([C@H](O)CN(C)C)c3)O[C@@](C)(C(F)(F)F)[C@H]2C)ccc(F)c1F]

(2R,3S,4S,5R)-3-(3,4-difluoro-2-methoxyphenyl)-
N-(2-((R)-2-(dimethylamino)-1-
hydroxyethyl)pyridin-4-yl)-4,5-dimethyl-5-
(trifluoromethyl)tetrahydrofuran-2-carboxamide

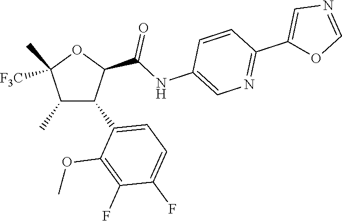 [SMILES: COc1c([C@H]2[C@H](C(=O)Nc3ccc(-c4cnco4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(oxazol-5-
yl)pyridin-3-yl)-5-
(trifluoromethyl)tetrahydrofuran-2-carboxamide

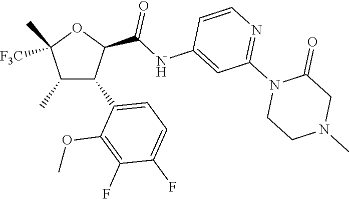 [SMILES: COc1c([C@H]2[C@H](C(=O)Nc3ccnc(N4CCN(C)CC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(4-methyl-2-
oxopiperazin-1-yl)pyridin-4-yl)-5-
(trifluoromethyl)tetrahydrofuran-2-carboxamide

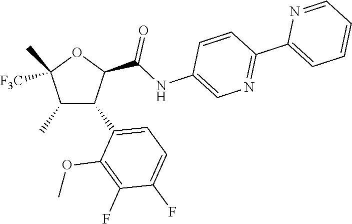 [SMILES: COc1c([C@H]2[C@H](C(=O)Nc3ccc(-c4ccccn4)nc3)O[C@@](C)(C(F)(F)F)[C@H]2C)ccc(F)c1F]

(2R,3S,4S,5R)-N-([2,2′-bipyridin]-5-yl)-3-(3,4-
difluoro-2-methoxyphenyl)-4,5-dimethyl-5-
(trifluoromethyl)tetrahydrofuran-2-carboxamide

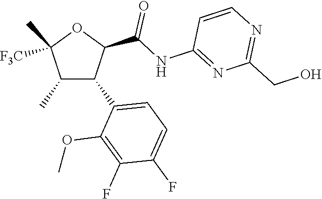 [SMILES: COc1c([C@H]2[C@H](C(=O)Nc3ccnc(CO)n3)O[C@@](C)(C(F)(F)F)[C@H]2C)ccc(F)c1F]

(2R,3S,4S,5R)-3-(3,4-difluoro-2-
methoxyphenyl)-N-(2-(hydroxymethyl)pyrimidin-
4-yl)-4,5-dimethyl-5-
(trifluoromethyl)tetrahydrofuran-2-carboxamide

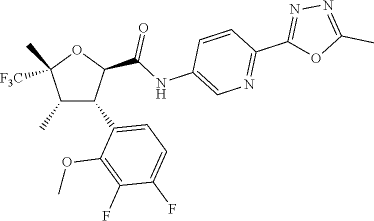 [SMILES: COc1c([C@H]2[C@H](C(=O)Nc3ccc(-c4nnc(C)o4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(5-methyl-
1,3,4-oxadiazol-2-yl)pyridin-3-yl)-5-
(trifluoromethyl)tetrahydrofuran-2-carboxamide

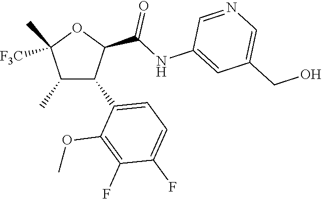 [SMILES: COc1c([C@H]2[C@H](C(=O)Nc3cncc(CO)c3)O[C@@](C)(C(F)(F)F)[C@H]2C)ccc(F)c1F]

(2R,3S,4S,5R)-3-(3,4-difluoro-2-
methoxyphenyl)-N-(5-(hydroxymethyl)pyridin-3-
yl)-4,5-dimethyl-5-
(trifluoromethyl)tetrahydrofuran-2-carboxamide

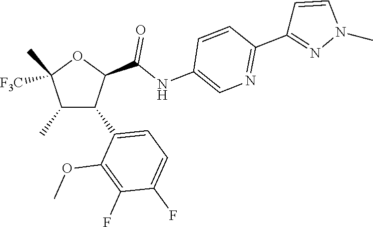 [SMILES: COc1c([C@H]2[C@H](C(=O)Nc3ccc(-c4ccn(C)n4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(1-methyl-
1H-pyrazol-3-yl)pyridin-3-yl)-5-
(trifluoromethyl)tetrahydrofuran-2-carboxamide

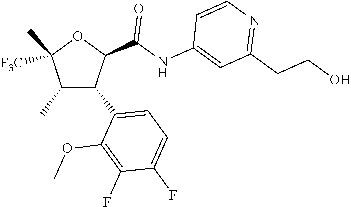 [SMILES: COc1c([C@H]2[C@H](C(=O)Nc3ccnc(CCO)c3)O[C@@](C)(C(F)(F)F)[C@H]2C)ccc(F)c1F]

(2R,3S,4S,5R)-3-(3,4-difluoro-2-
methoxyphenyl)-N-(2-(2-hydroxyethyl)pyridin-4-
yl)-4,5-dimethyl-5-
(trifluoromethyl)tetrahydrofuran-2-carboxamide

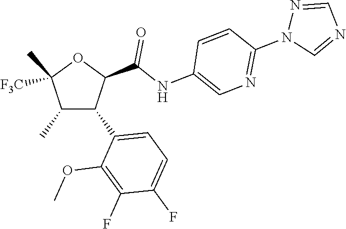 [SMILES: COc1c([C@H]2[C@H](C(=O)Nc3ccc(-n4cncn4)nc3)O[C@@](C)(C(F)(F)F)[C@H]2C)ccc(F)c1F]

(2R,3S,4S,5R)-N-(6-(1H-1,2,4-triazol-1-
yl)pyridin-3-yl)-3-(3,4-difluoro-2-
methoxyphenyl)-4,5-dimethyl-5-
(trifluoromethyl)tetrahydrofuran-2-carboxamide

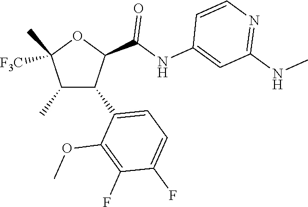 [SMILES: CNc1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-
methoxyphenyl)-4,5-dimethyl-N-(2-
(methylamino)pyridin-4-yl)-5-
(trifluoromethyl)tetrahydrofuran-2-carboxamide

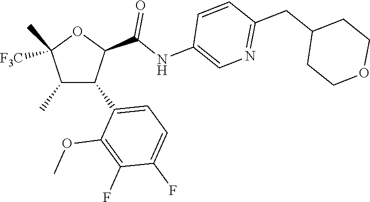 [SMILES: COc1c([C@H]2[C@H](C(=O)Nc3ccc(CC4CCOCC4)nc3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(6-((tetrahydro-2H-pyran-4-
yl)methyl)pyridin-3-yl)-5-
(trifluoromethyl)tetrahydrofuran-2-carboxamide

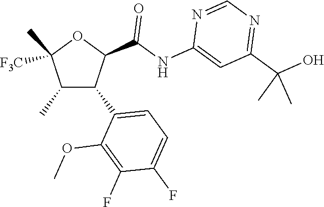 [SMILES: COc1c([C@H]2[C@H](C(=O)Nc3cc(C(C)(C)O)ncn3)O[C@@](C)(C(F)(F)F)[C@H]2C)ccc(F)c1F]

(2R,3S,4S,5R)-3-(3,4-difluoro-2-
methoxyphenyl)-N-(6-(2-hydroxypropan-2-
yl)pyrimidin-4-yl)-4,5-dimethyl-5-
(trifluoromethyl)tetrahydrofuran-2-carboxamide

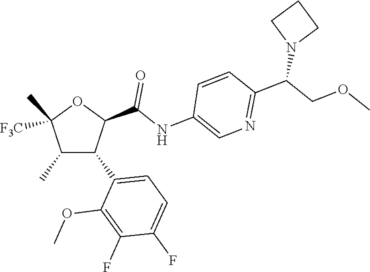 [SMILES: COC[C@H](c1ccc(NC(=O)[C@@H]2O[C@@](C)(C(F)(F)F)[C@@H](C)[C@H]2c2ccc(F)c(F)c2OC)cn1)N1CCC1]

(2R,3S,4S,5R)-N-(6-((S)-1-(azetidin-1-yl)-2-
methoxyethyl)pyridin-3-yl)-3-(3,4-difluoro-2-
methoxyphenyl)-4,5-dimethyl-5-
(trifluoromethyl)tetrahydrofuran-2-carboxamide

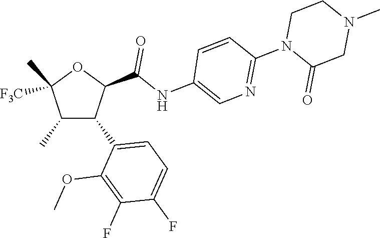 [SMILES: COc1c([C@H]2[C@H](C(=O)Nc3ccc(N4CCN(C)CC4=O)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(4-methyl-2-
oxopiperazin-1-yl)pyridin-3-yl)-5-
(trifluoromethyl)tetrahydrofuran-2-carboxamide

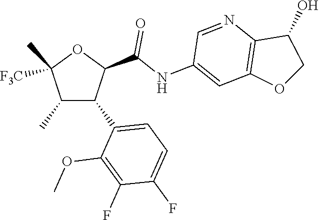 [SMILES: COc1c([C@H]2[C@H](C(=O)Nc3cnc4c(c3)OC[C@H]4O)O[C@@](C)(C(F)(F)F)[C@H]2C)ccc(F)c1F]

(2R,3S,4S,5R)-3-(3,4-difluoro-2-
methoxyphenyl)-N-((S)-3-hydroxy-2,3-
dihydrofuro[3,2-b]pyridin-6-yl)-4,5-dimethyl-5-
(trifluoromethyl)tetrahydrofuran-2-carboxamide

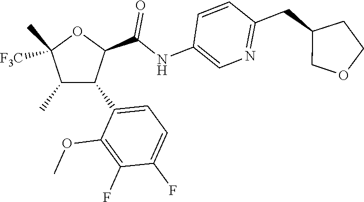 [SMILES: COc1c([C@H]2[C@H](C(=O)Nc3ccc(C[C@H]4CCOC4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(((S)-
tetrahydrofuran-3-yl)methyl)pyridin-3-yl)-5-
(trifluoromethyl)tetrahydrofuran-2-carboxamide

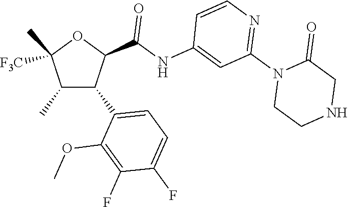 [SMILES: COc1c([C@H]2[C@H](C(=O)Nc3ccnc(N4CCNCC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(2-
oxopiperazin-1-yl)pyridin-4-yl)-5-
(trifluoromethyl)tetrahydrofuran-2-carboxamide

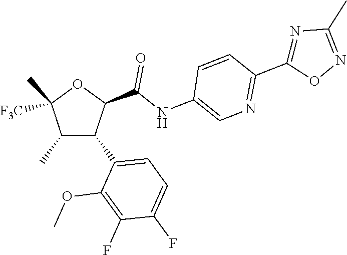 [SMILES: COc1c([C@H]2[C@H](C(=O)Nc3ccc(-c4nc(C)no4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(3-methyl-
1,2,4-oxadiazol-5-yl)pyridin-3-yl)-5-
(trifluoromethyl)tetrahydrofuran-2-carboxamide

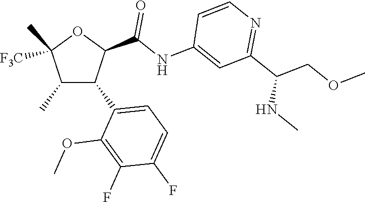 [SMILES: CN[C@@H](COC)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-N-
(2-((R)-2-methoxy-1-(methylamino)ethyl)pyridin-4-
yl)-4,5-dimethyl-5-(trifluoromethyl)tetrahydrofuran-2-
carboxamide

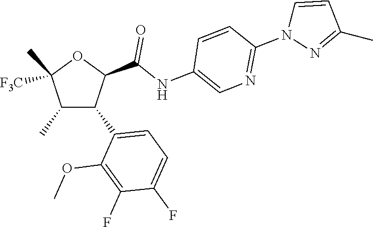 [SMILES: COc1c([C@H]2[C@H](C(=O)Nc3ccc(-n4ccc(C)n4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(3-methyl-
1H-pyrazol-1-yl)pyridin-3-yl)-5-
(trifluoromethyl)tetrahydrofuran-2-carboxamide

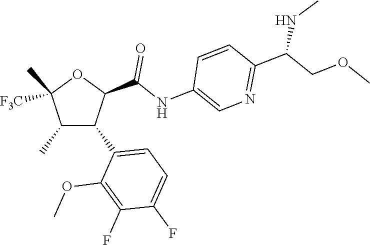 [SMILES: CN[C@H](COC)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-
methoxyphenyl)-N-(6-((S)-2-methoxy-1-
(methylamino)ethyl)pyridin-3-yl)-4,5-dimethyl-5-
(trifluoromethyl)tetrahydrofuran-2-carboxamide

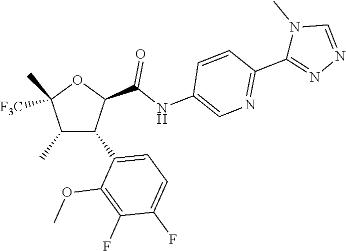 [SMILES: COc1c([C@H]2[C@H](C(=O)Nc3ccc(-c4nncn4C)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(4-methyl-
4H-1,2,4-triazol-3-yl)pyridin-3-yl)-5-
(trifluoromethyl)tetrahydrofuran-2-carboxamide

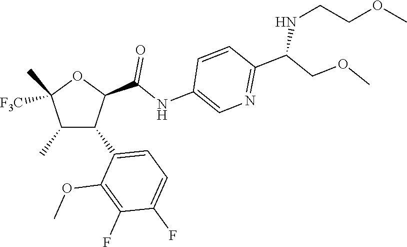 [SMILES: COCCN[C@H](COC)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-N-(6-
((S)-2-methoxy-1-((2-
methoxyethyl)amino)ethyl)pyridin-3-yl)-4,5-dimethyl-5-
(trifluoromethyl)tetrahydrofuran-2-carboxamide

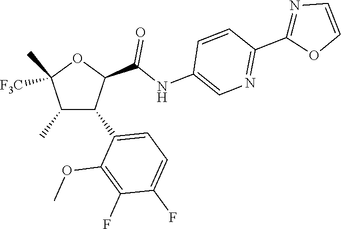 [SMILES: COc1c([C@H]2[C@H](C(=O)Nc3ccc(-c4ncco4)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-(oxazol-2-
yl)pyridin-3-yl)-5-
(trifluoromethyl)tetrahydrofuran-2-carboxamide

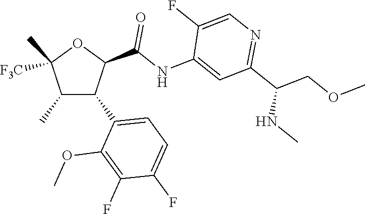 [SMILES: CN[C@@H](COC)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-3-(3,4-difluoro-2-methoxyphenyl)-N-
(5-fluoro-2-((R)-2-methoxy-1-
(methylamino)ethyl)pyridin-4-yl)-4,5-dimethyl-5-
(trifluoromethyl)tetrahydrofuran-2-carboxamide

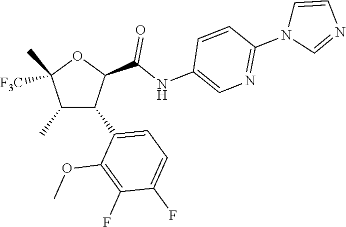 [SMILES: COc1c([C@H]2[C@H](C(=O)Nc3ccc(-n4ccnc4)nc3)O[C@@](C)(C(F)(F)F)[C@H]2C)ccc(F)c1F]

(2R,3S,4S,5R)-N-(6-(1H-imidazol-1-yl)pyridin-
3-yl)-3-(3,4-difluoro-2-methoxyphenyl)-4,5-
dimethyl-5-(trifluoromethyl)tetrahydrofuran-2-
carboxamide

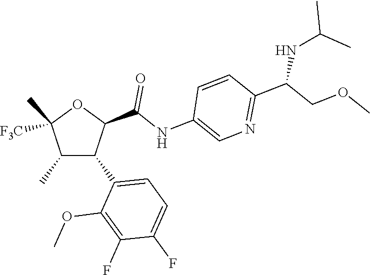 [SMILES: COC[C@@H](NC(C)C)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-N-(6-
((S)-1-(isopropylamino)-2-methoxyethyl)pyridin-3-yl)-
4,5-dimethyl-5-(trifluoromethyl)tetrahydrofuran-2-
carboxamide

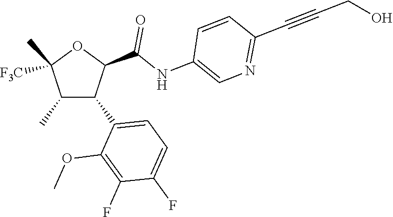 [SMILES: COc1c([C@H]2[C@H](C(=O)Nc3ccc(C#CCO)nc3)O[C@@](C)(C(F)(F)F)[C@H]2C)ccc(F)c1F]

(2R,3S,4S,5R)-3-(3,4-difluoro-2-
methoxyphenyl)-N-(6-(3-hydroxyprop-1-yn-1-
yl)pyridin-3-yl)-4,5-dimethyl-5-
(trifluoromethyl)tetrahydrofuran-2-carboxamide

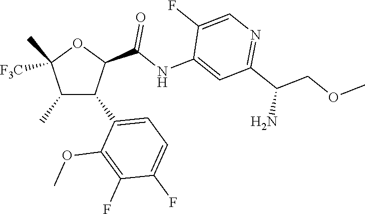 [SMILES: COC[C@H](N)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-N-(2-((R)-1-amino-2-methoxyethyl)-
5-fluoropyridin-4-yl)-3-(3,4-difluoro-2-
methoxyphenyl)-4,5-dimethyl-5-
(trifluoromethyl)tetrahydrofuran-2-carboxamide

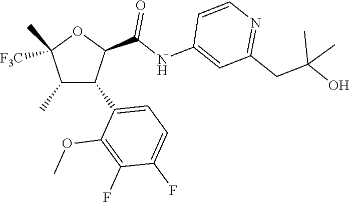 [SMILES: COc1c([C@H]2[C@H](C(=O)Nc3ccnc(CC(C)(C)O)c3)O[C@@](C)(C(F)(F)F)[C@H]2C)ccc(F)c1F]

(2R,3S,4S,5R)-3-(3,4-difluoro-2-
methoxyphenyl)-N-(2-(2-hydroxy-2-
methylpropyl)pyridin-4-yl)-4,5-dimethyl-5-
(trifluoromethyl)tetrahydrofuran-2-carboxamide

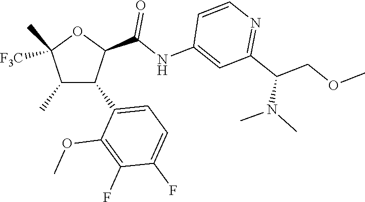 [SMILES: COC[C@@H](c1cc(NC(=O)[C@@H]2O[C@@](C)(C(F)(F)F)[C@@H](C)[C@H]2c2ccc(F)c(F)c2OC)ccn1)N(C)C]

(2R,3S,4S,5R)-3-(3,4-difluoro-2-methoxyphenyl)-N-
(2-((R)-1-(dimethylamino)-2-methoxyethyl)pyridin-4-
yl)-4,5-dimethyl-5-(trifluoromethyl)tetrahydrofuran-2-
carboxamide

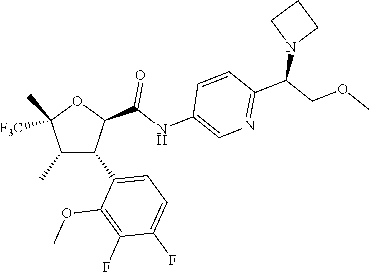 [SMILES: COC[C@@H](c1ccc(NC(=O)[C@@H]2O[C@@](C)(C(F)(F)F)[C@@H](C)[C@H]2c2ccc(F)c(F)c2OC)cn1)N1CCC1]

(2R,3S,4S,5R)-N-(6-((R)-1-(azetidin-1-yl)-2-
methoxyethyl)pyridin-3-yl)-3-(3,4-difluoro-2-
methoxyphenyl)-4,5-dimethyl-5-
(trifluoromethyl)tetrahydrofuran-2-carboxamide

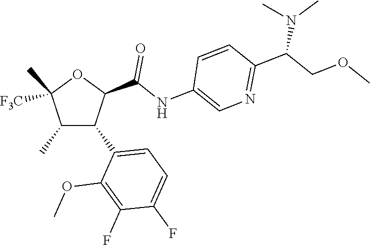 [SMILES: COC[C@H](c1ccc(NC(=O)[C@@H]2O[C@@](C)(C(F)(F)F)[C@@H](C)[C@H]2c2ccc(F)c(F)c2OC)cn1)N(C)C]

(2R,3S,4S,5R)-3-(3,4-difluoro-2-methoxyphenyl)-N-(6-
((S)-1-(dimethylamino)-2-methoxyethyl)pyridin-3-yl)-
4,5-dimethyl-5-(trifluoromethyl)tetrahydrofuran-2-
carboxamide

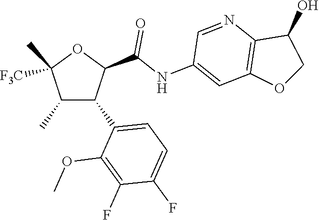 [SMILES: COc1c([C@H]2[C@H](C(=O)Nc3cnc4c(c3)OC[C@@H]4O)O[C@@](C)(C(F)(F)F)[C@H]2C)ccc(F)c1F]

(2R,3S,4S,5R)-3-(3,4-difluoro-2-
methoxyphenyl)-N-((R)-3-hydroxy-2,3-
dihydrofuro[3,2-b]pyridin-6-yl)-4,5-dimethyl-5-
(trifluoromethyl)tetrahydrofuran-2-carboxamide

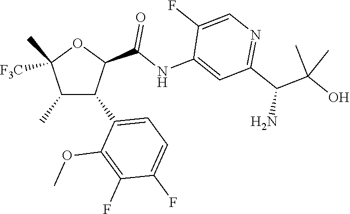 [SMILES: COc1c([C@H]2[C@H](C(=O)Nc3cc([C@@H](N)C(C)(C)O)ncc3F)O[C@@](C)(C(F)(F)F)[C@H]2C)ccc(F)c1F]

(2R,3S,4S,5R)-N-(2-((R)-1-amino-2-hydroxy-2-
methylpropyl)-5-fluoropyridin-4-yl)-3-(3,4-difluoro-2-
methoxyphenyl)-4,5-dimethyl-5-
(trifluoromethyl)tetrahydrofuran-2-carboxamide

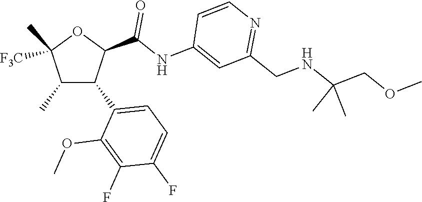 [SMILES: COCC(C)(C)NCc1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-N-
(2-(((1-methoxy-2-methylpropan-2-
yl)amino)methyl)pyridin-4-yl)-4,5-dimethyl-5-
(trifluoromethyl)tetrahydrofuran-2-carboxamide

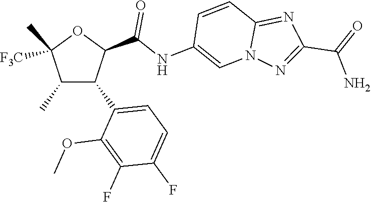 [SMILES: COc1c([C@H]2[C@H](C(=O)Nc3ccc4nc(C(N)=O)nn4c3)O[C@@](C)(C(F)(F)F)[C@H]2C)ccc(F)c1F]

6-((2R,3S,4S,5R)-3-(3,4-difluoro-2-methoxyphenyl)-4,5-
dimethyl-5-(trifluoromethyl)tetrahydrofuran-2-
carboxamido)-[1,2,4]triazolo[1,5-a]pyridine-2-
carboxamide

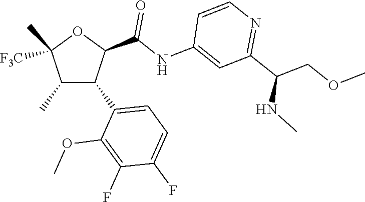 [SMILES: CN[C@H](COC)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-N-
(2-((S)-2-methoxy-1-(methylamino)ethyl)pyridin-4-
yl)-4,5-dimethyl-5-(trifluoromethyl)tetrahydrofuran-2-
carboxamide

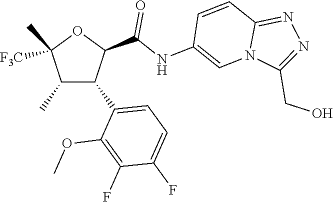 [SMILES: COc1c([C@H]2[C@H](C(=O)Nc3ccc4nnc(CO)n4c3)O[C@@](C)(C(F)(F)F)[C@H]2C)ccc(F)c1F]

(2R,3S,4S,5R)-3-(3,4-difluoro-2-methoxyphenyl)-N-(3-
(hydroxymethyl)-[1,2,4]triazolo[4,3-a]pyridin-6-yl)-4,5-
dimethyl-5-(trifluoromethyl)tetrahydrofuran-2-
carboxamide

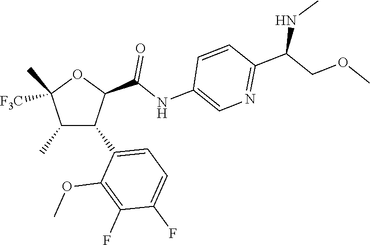 [SMILES: CN[C@@H](COC)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-
methoxyphenyl)-N-(6-((R)-2-methoxy-1-
(methylamino)ethyl)pyridin-3-yl)-4,5-dimethyl-5-
(trifluoromethyl)tetrahydrofuran-2-carboxamide

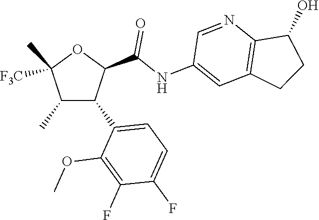 [SMILES: COc1c([C@H]2[C@H](C(=O)Nc3cnc4c(c3)CC[C@H]4O)O[C@@](C)(C(F)(F)F)[C@H]2C)ccc(F)c1F]

(2R,3S,4S,5R)-3-(3,4-difluoro-2-methoxyphenyl)-N-
((R)-7-hydroxy-6,7-dihydro-5H-cyclopenta[b]pyridin-
3-yl)-4,5-dimethyl-5-(trifluoromethyl)tetrahydrofuran-
2-carboxamide

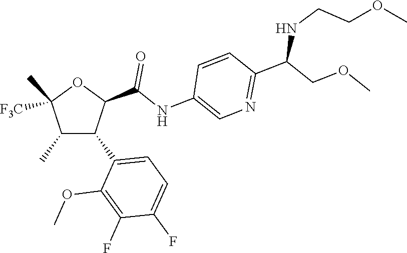 [SMILES: COCCN[C@@H](COC)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-N-(6-
((R)-2-methoxy-1-((2-methoxyethyl)amino)ethyl)pyridin-
3-yl)-4,5-dimethyl-5-(trifluoromethyl)tetrahydrofuran-2-
carboxamide

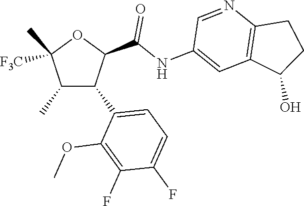 [SMILES: COc1c([C@H]2[C@H](C(=O)Nc3cnc4c(c3)[C@@H](O)CC4)O[C@@](C)(C(F)(F)F)[C@H]2C)ccc(F)c1F]

(2R,3S,4S,5R)-3-(3,4-difluoro-2-methoxyphenyl)-
N-((S)-5-hydroxy-6,7-dihydro-5H-
cyclopenta[b]pyridin-3-yl)-4,5-dimethyl-5-
(trifluoromethyl)tetrahydrofuran-2-carboxamide

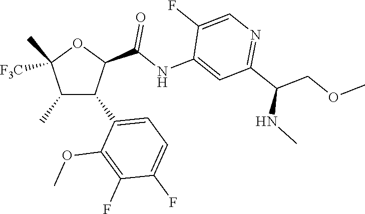 [SMILES: CN[C@H](COC)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-3-(3,4-difluoro-2-methoxyphenyl)-N-
(5-fluoro-2-((S)-2-methoxy-1-
(methylamino)ethyl)pyridin-4-yl)-4,5-dimethyl-5-
(trifluoromethyl)tetrahydrofuran-2-carboxamide

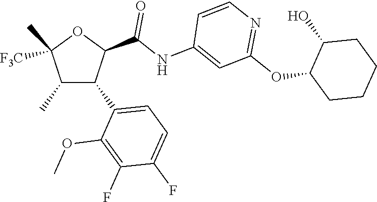 [SMILES: COc1c([C@H]2[C@H](C(=O)Nc3ccnc(O[C@H]4CCCC[C@H]4O)c3)O[C@@](C)(C(F)(F)F)[C@H]2C)ccc(F)c1F]

(2R,3S,4S,5R)-3-(3,4-difluoro-2-methoxyphenyl)-
N-(2-(((1S,2R)-2-hydroxycyclohexyl)oxy)pyridin-4-
yl)-4,5-dimethyl-5-(trifluoromethyl)tetrahydrofuran-
2-carboxamide

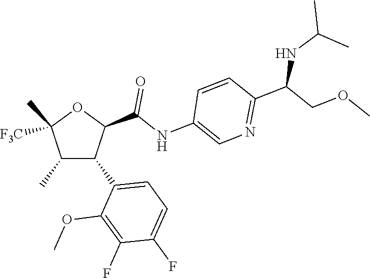 [SMILES: COC[C@H](NC(C)C)c1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-methoxyphenyl)-N-(6-
((R)-1-(isopropylamino)-2-methoxyethyl)pyridin-3-yl)-
4,5-dimethyl-5-(trifluoromethyl)tetrahydrofuran-2-
carboxamide

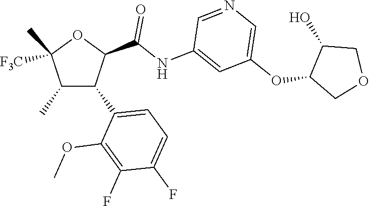 [SMILES: COc1c([C@H]2[C@H](C(=O)Nc3cncc(O[C@H]4COC[C@H]4O)c3)O[C@@](C)(C(F)(F)F)[C@H]2C)ccc(F)c1F]

(2R,3S,4S,5R)-3-(3,4-difluoro-2-methoxyphenyl)-
N-(5-(((3S,4R)-4-hydroxytetrahydrofuran-3-
yl)oxy)pyridin-3-yl)-4,5-dimethyl-5-
(trifluoromethyl)tetrahydrofuran-2-carboxamide

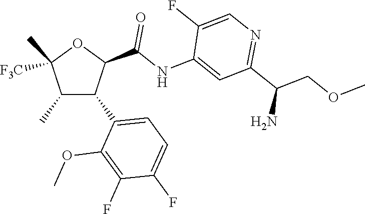 [SMILES: COC[C@@H](N)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-N-(2-((S)-1-amino-2-methoxyethyl)-
5-fluoropyridin-4-yl)-3-(3,4-difluoro-2-
methoxyphenyl)-4,5-dimethyl-5-
(trifluoromethyl)tetrahydrofuran-2-carboxamide

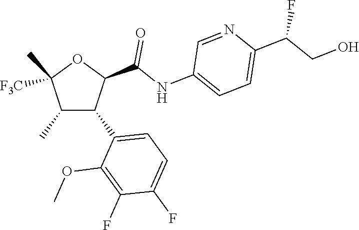 [SMILES: COc1c([C@H]2[C@H](C(=O)Nc3ccc([C@H](F)CO)nc3)O[C@@](C)(C(F)(F)F)[C@H]2C)ccc(F)c1F]

(2R,3S,4S,5R)-3-(3,4-difluoro-2-methoxyphenyl)-
N-(6-((S)-1-fluoro-2-hydroxyethyl)pyridin-3-yl)-4,5-
dimethyl-5-(trifluoromethyl)tetrahydrofuran-2-
carboxamide

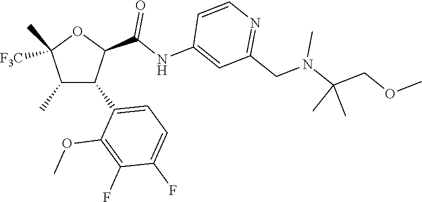 [SMILES: COCC(C)(C)N(C)Cc1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-N-
(2-(((1-methoxy-2-methylpropan-2-
yl)(methyl)amino)methyl)pyridin-4-yl)-4,5-dimethyl-
5-(trifluoromethyl)tetrahydrofuran-2-carboxamide

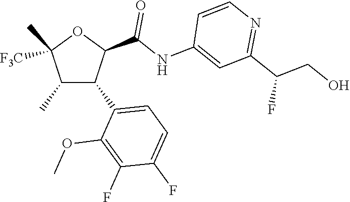 [SMILES: COc1c([C@H]2[C@H](C(=O)Nc3ccnc([C@@H](F)CO)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1-fluoro-2-hydroxyethyl)pyridin-4-yl)-
4,5-dimethyl-5-(trifluoromethyl)tetrahydrofuran-2-
carboxamide

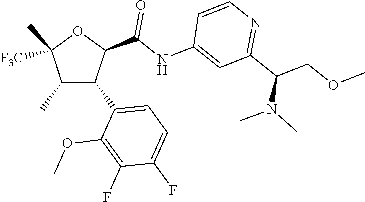 [SMILES: COC[C@H](c1cc(NC(=O)[C@@H]2O[C@@](C)(C(F)(F)F)[C@@H](C)[C@H]2c2ccc(F)c(F)c2OC)ccn1)N(C)C]

(2R,3S,4S,5R)-3-(3,4-difluoro-2-methoxyphenyl)-N-
(2-((S)-1-(dimethylamino)-2-methoxyethyl)pyridin-4-
yl)-4,5-dimethyl-5-(trifluoromethyl)tetrahydrofuran-2-
carboxamide

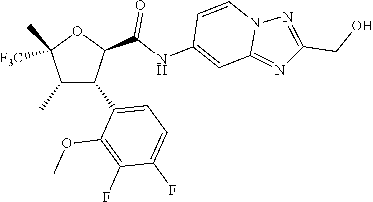 [SMILES: COc1c([C@H]2[C@H](C(=O)Nc3ccn4nc(CO)nc4c3)O[C@@](C)(C(F)(F)F)[C@H]2C)ccc(F)c1F]

(2R,3S,4S,5R)-3-(3,4-difluoro-2-methoxyphenyl)-N-(2-
(hydroxymethyl)-[1,2,4]triazolo[1,5-a]pyridin-7-yl)-4,5-
dimethyl-5-(trifluoromethyl)tetrahydrofuran-2-
carboxamide

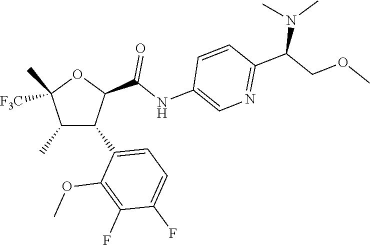 [SMILES: COC[C@@H](c1ccc(NC(=O)[C@@H]2O[C@@](C)(C(F)(F)F)[C@@H](C)[C@H]2c2ccc(F)c(F)c2OC)cn1)N(C)C]

(2R,3S,4S,5R)-3-(3,4-difluoro-2-methoxyphenyl)-N-(6-
((R)-1-(dimethylamino)-2-methoxyethyl)pyridin-3-yl)-
4,5-dimethyl-5-(trifluoromethyl)tetrahydrofuran-2-
carboxamide

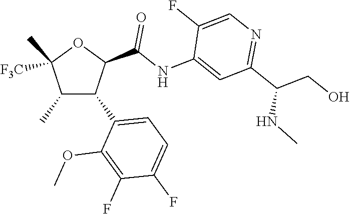 [SMILES: CN[C@@H](CO)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-3-(3,4-difluoro-2-methoxyphenyl)-N-
(5-fluoro-2-((R)-2-hydroxy-1-
(methylamino)ethyl)pyridin-4-yl)-4,5-dimethyl-5-
(trifluoromethyl)tetrahydrofuran-2-carboxamide

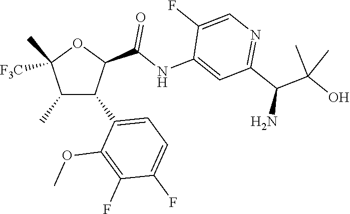 [SMILES: COc1c([C@H]2[C@H](C(=O)Nc3cc([C@H](N)C(C)(C)O)ncc3F)O[C@@](C)(C(F)(F)F)[C@H]2C)ccc(F)c1F]

(2R,3S,4S,5R)-N-(2-((S)-1-amino-2-hydroxy-2-
methylpropyl)-5-fluoropyridin-4-yl)-3-(3,4-difluoro-2-
methoxyphenyl)-4,5-dimethyl-5-
(trifluoromethyl)tetrahydrofuran-2-carboxamide

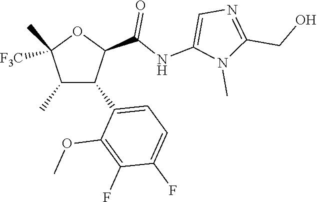 [SMILES: COc1c([C@H]2[C@H](C(=O)Nc3cnc(CO)n3C)O[C@@](C)(C(F)(F)F)[C@H]2C)ccc(F)c1F]

(2R,3S,4S,5R)-3-(3,4-difluoro-2-methoxyphenyl)-N-
(2-(hydroxymethyl)-1-methyl-1H-imidazol-5-yl)-4,5-
dimethyl-5-(trifluoromethyl)tetrahydrofuran-2-
carboxamide

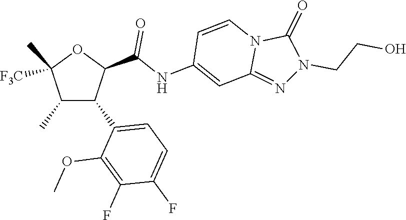 [SMILES: COc1c([C@H]2[C@H](C(=O)Nc3ccn4c(=O)n(CCO)nc4c3)O[C@@](C)(C(F)(F)F)[C@H]2C)ccc(F)c1F]

(2R,3S,4S,5R)-3-(3,4-difluoro-2-methoxyphenyl)-N-(2-
(2-hydroxyethyl)-3-oxo-2,3-dihydro-[1,2,4]triazolo[4,3-
a]pyridin-7-yl)-4,5-dimethyl-5-
(trifluoromethyl)tetrahydrofuran-2-carboxamide

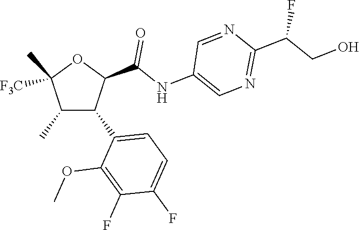 [SMILES: COc1c([C@H]2[C@H](C(=O)Nc3cnc([C@H](F)CO)nc3)O[C@@](C)(C(F)(F)F)[C@H]2C)ccc(F)c1F]

(2R,3S,4S,5R)-3-(3,4-difluoro-2-methoxyphenyl)-N-(2-
((S)-1-fluoro-2-hydroxyethyl)pyrimidin-5-yl)-4,5-
dimethyl-5-(trifluoromethyl)tetrahydrofuran-2-
carboxamide

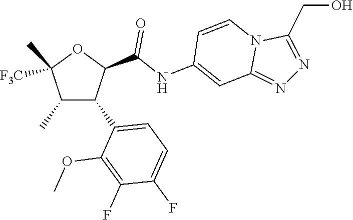 [SMILES: COc1c([C@H]2[C@H](C(=O)Nc3ccn4c(CO)nnc4c3)O[C@@](C)(C(F)(F)F)[C@H]2C)ccc(F)c1F]

(2R,3S,4S,5R)-3-(3,4-difluoro-2-methoxyphenyl)-N-(3-
(hydroxymethyl)-[1,2,4]triazolo[4,3-a]pyridin-7-yl)-4,5-
dimethyl-5-(trifluoromethyl)tetrahydrofuran-2-
carboxamide

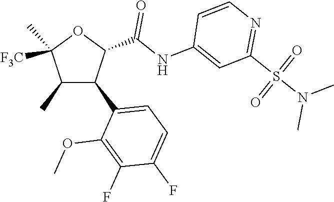 [SMILES: COc1c([C@@H]2[C@@H](C(=O)Nc3ccnc(S(=O)(=O)N(C)C)c3)O[C@](C)(C(F)(F)F)[C@@H]2C)ccc(F)c1F]

(2S,3R,4R,5S)-3-(3,4-difluoro-2-methoxyphenyl)-N-(2-
(N,N-dimethylsulfamoyl)pyridin-4-yl)-4,5-dimethyl-5-
(trifluoromethyl)tetrahydrofuran-2-carboxamide

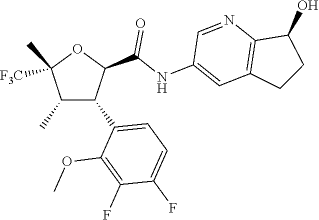 [SMILES: COc1c([C@H]2[C@H](C(=O)Nc3cnc4c(c3)CC[C@@H]4O)O[C@@](C)(C(F)(F)F)[C@H]2C)ccc(F)c1F]

(2R,3S,4S,5R)-3-(3,4-difluoro-2-methoxyphenyl)-
N-((S)-7-hydroxy-6,7-dihydro-5H-
cyclopenta[b]pyridin-3-yl)-4,5-dimethyl-5-
(trifluoromethyl)tetrahydrofuran-2-carboxamide

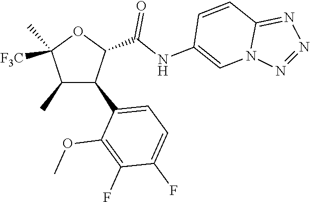 [SMILES: COc1c([C@@H]2[C@@H](C(=O)Nc3ccc4nnnn4c3)O[C@](C)(C(F)(F)F)[C@@H]2C)ccc(F)c1F]

(2S,3R,4R,5S)-3-(3,4-difluoro-2-methoxyphenyl)-4,5-
dimethyl-N-(tetrazolo[1,5-a]pyridin-6-yl)-5-
(trifluoromethyl)tetrahydrofuran-2-carboxamide

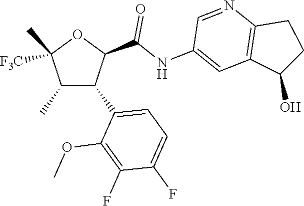 [SMILES: COc1c([C@H]2[C@H](C(=O)Nc3cnc4c(c3)[C@H](O)CC4)O[C@@](C)(C(F)(F)F)[C@H]2C)ccc(F)c1F]

(2R,3S,4S,5R)-3-(3,4-difluoro-2-methoxyphenyl)-
N-((R)-5-hydroxy-6,7-dihydro-5H-
cyclopenta[b]pyridin-3-yl)-4,5-dimethyl-5-
(trifluoromethyl)tetrahydrofuran-2-carboxamide

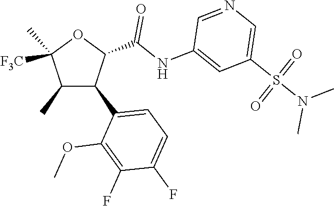 [SMILES: COc1c([C@@H]2[C@@H](C(=O)Nc3cncc(S(=O)(=O)N(C)C)c3)O[C@](C)(C(F)(F)F)[C@@H]2C)ccc(F)c1F]

(2S,3R,4R,5S)-3-(3,4-difluoro-2-methoxyphenyl)-N-(5-
(N,N-dimethylsulfamoyl)pyridin-3-yl)-4,5-dimethyl-5-
(trifluoromethyl)tetrahydrofuran-2-carboxamide

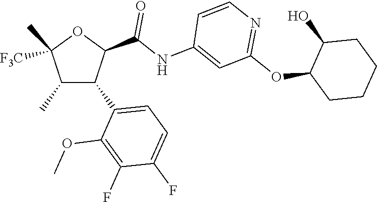 [SMILES: COc1c([C@H]2[C@H](C(=O)Nc3ccnc(O[C@@H]4CCCC[C@@H]4O)c3)O[C@@](C)(C(F)(F)F)[C@H]2C)ccc(F)c1F]

(2R,3S,4S,5R)-3-(3,4-difluoro-2-methoxyphenyl)-
N-(2-(((1R,2S)-2-hydroxycyclohexyl)oxy)pyridin-4-
yl)-4,5-dimethyl-5-(trifluoromethyl)tetrahydrofuran-
2-carboxamide

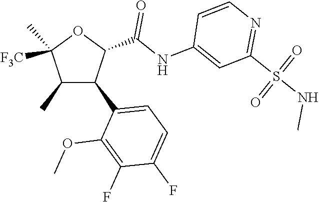 [SMILES: CNS(=O)(=O)c1cc(NC(=O)[C@H]2O[C@](C)(C(F)(F)F)[C@H](C)[C@@H]2c2ccc(F)c(F)c2OC)ccn1]

(2S,3R,4R,5S)-3-(3,4-difluoro-2-
methoxyphenyl)-4,5-dimethyl-N-(2-(N-
methylsulfamoyl)pyridin-4-yl)-5-
(trifluoromethyl)tetrahydrofuran-2-carboxamide

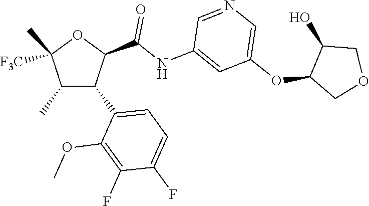 [SMILES: COc1c([C@H]2[C@H](C(=O)Nc3cncc(O[C@@H]4COC[C@@H]4O)c3)O[C@@](C)(C(F)(F)F)[C@H]2C)ccc(F)c1F]

(2R,3S,4S,5R)-3-(3,4-difluoro-2-methoxyphenyl)-
N-(5-(((3R,4S)-4-hydroxytetrahydrofuran-3-
yl)oxy)pyridin-3-yl)-4,5-dimethyl-5-
(trifluoromethyl)tetrahydrofuran-2-carboxamide

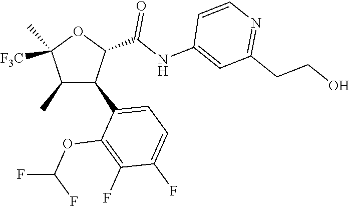 [SMILES: C[C@@H]1[C@H](c2ccc(F)c(F)c2OC(F)F)[C@@H](C(=O)Nc2ccnc(CCO)c2)O[C@]1(C)C(F)(F)F]

(2S,3R,4R,5S)-3-(2-(difluoromethoxy)-3,4-
difluorophenyl)-N-(2-(2-hydroxyethyl)pyridin-4-yl)-4,5-
dimethyl-5-(trifluoromethyl)tetrahydrofuran-2-
carboxamide

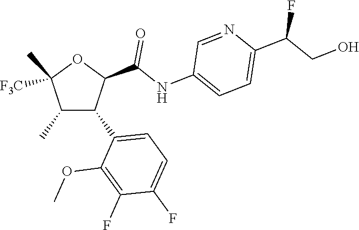 [SMILES: COc1c([C@H]2[C@H](C(=O)Nc3ccc([C@@H](F)CO)nc3)O[C@@](C)(C(F)(F)F)[C@H]2C)ccc(F)c1F]

(2R,3S,4S,5R)-3-(3,4-difluoro-2-methoxyphenyl)-
N-(6-((R)-1-fluoro-2-hydroxyethyl)pyridin-3-yl)-
4,5-dimethyl-5-(trifluoromethyl)tetrahydrofuran-2-
carboxamide

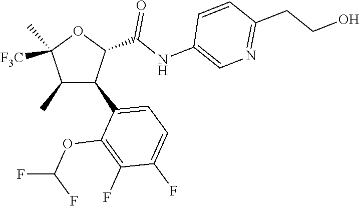 [SMILES: C[C@@H]1[C@H](c2ccc(F)c(F)c2OC(F)F)[C@@H](C(=O)Nc2ccc(CCO)nc2)O[C@]1(C)C(F)(F)F]

(2S,3R,4R,5S)-3-(2-(difluoromethoxy)-3,4-
difluorophenyl)-N-(6-(2-hydroxyethyl)pyridin-3-yl)-4,5-
dimethyl-5-(trifluoromethyl)tetrahydrofuran-2-
carboxamide

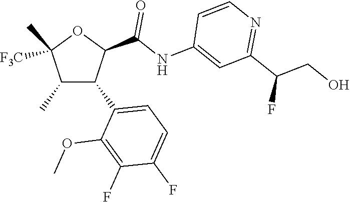 [SMILES: COc1c([C@H]2[C@H](C(=O)Nc3ccnc([C@H](F)CO)c3)O[C@@](C)(C(F)(F)F)[C@H]2C)ccc(F)c1F]

(2R,3S,4S,5R)-3-(3,4-difluoro-2-methoxyphenyl)-
N-(2-((S)-1-fluoro-2-hydroxyethyl)pyridin-4-yl)-4,5-
dimethyl-5-(trifluoromethyl)tetrahydrofuran-2-
carboxamide

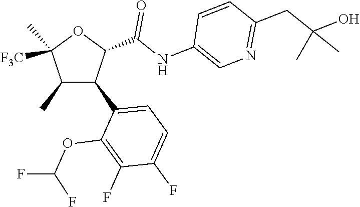 [SMILES: C[C@@H]1[C@H](c2ccc(F)c(F)c2OC(F)F)[C@@H](C(=O)Nc2ccc(CC(C)(C)O)nc2)O[C@]1(C)C(F)(F)F]

(2S,3R,4R,5S)-3-(2-(difluoromethoxy)-3,4-
difluorophenyl)-N-(6-(2-hydroxy-2-methylpropyl)pyridin-
3-yl)-4,5-dimethyl-5-(trifluoromethyl)tetrahydrofuran-2-
carboxamide

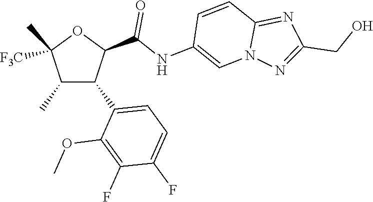 [SMILES: COc1c([C@H]2[C@H](C(=O)Nc3ccc4nc(CO)nn4c3)O[C@@](C)(C(F)(F)F)[C@H]2C)ccc(F)c1F]

(2R,3S,4S,5R)-3-(3,4-difluoro-2-methoxyphenyl)-N-(2-
(hydroxymethyl)-[1,2,4]triazolo[1,5-a]pyridin-6-yl)-4,5-
dimethyl-5-(trifluoromethyl)tetrahydrofuran-2-
carboxamide

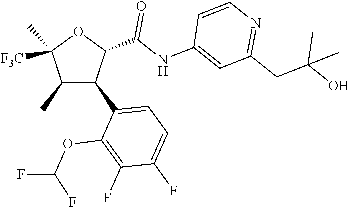 [SMILES: C[C@@H]1[C@H](c2ccc(F)c(F)c2OC(F)F)[C@@H](C(=O)Nc2ccnc(CC(C)(C)O)c2)O[C@]1(C)C(F)(F)F]

(2S,3R,4R,5S)-3-(2-(difluoromethoxy)-3,4-
difluorophenyl)-N-(2-(2-hydroxy-2-
methylpropyl)pyridin-4-yl)-4,5-dimethyl-5-
(trifluoromethyl)tetrahydrofuran-2-carboxamide

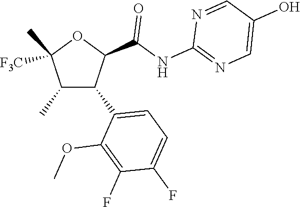 [SMILES: COc1c([C@H]2[C@H](C(=O)Nc3ncc(O)cn3)O[C@@](C)(C(F)(F)F)[C@H]2C)ccc(F)c1F]

(2R,3S,4S,5R)-3-(3,4-difluoro-2-methoxyphenyl)-N-(5-
hydroxypyrimidin-2-yl)-4,5-dimethyl-5-
(trifluoromethyl)tetrahydrofuran-2-carboxamide

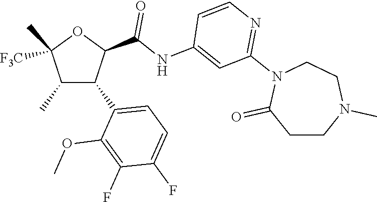 [SMILES: COc1c([C@H]2[C@H](C(=O)Nc3ccnc(N4CCN(C)CCC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(4-methyl-7-
oxo-1,4-diazepan-1-yl)pyridin-4-yl)-5-
(trifluoromethyl)tetrahydrofuran-2-carboxamide

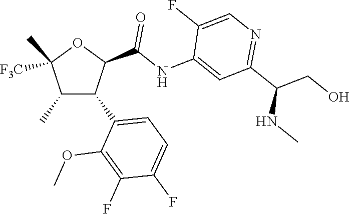 [SMILES: CN[C@H](CO)c1cc(NC(=O)[C@@H]2O[C@@](C)(C(F)(F)F)[C@@H](C)[C@H]2c2ccc(F)c(F)c2OC)c(F)cn1]

(2R,3S,4S,5R)-3-(3,4-difluoro-2-methoxyphenyl)-N-
(5-fluoro-2-((S)-2-hydroxy-1-
(methylamino)ethyl)pyridin-4-yl)-4,5-dimethyl-5-
(trifluoromethyl)tetrahydrofuran-2-carboxamide

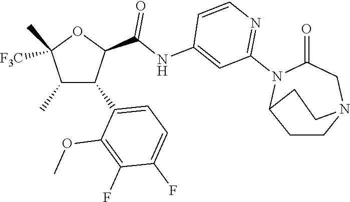 [SMILES: COc1c([C@H]2[C@H](C(=O)Nc3ccnc(N4C(=O)CN5CCC4CC5)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(3-oxo-1,4-
diazabicyclo[3.2.2]nonan-4-yl)pyridin-4-yl)-5-
(trifluoromethyl)tetrahydrofuran-2-carboxamide

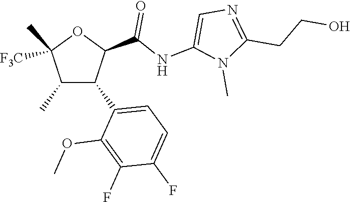 [SMILES: COc1c([C@H]2[C@H](C(=O)Nc3cnc(CCO)n3C)O[C@@](C)(C(F)(F)F)[C@H]2C)ccc(F)c1F]

(2R,3S,4S,5R)-3-(3,4-difluoro-2-methoxyphenyl)-N-
(2-(2-hydroxyethyl)-1-methyl-1H-imidazol-5-yl)-4,5-
dimethyl-5-(trifluoromethyl)tetrahydrofuran-2-
carboxamide

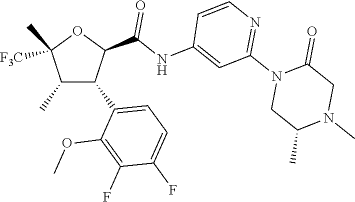 [SMILES: COc1c([C@H]2[C@H](C(=O)Nc3ccnc(N4C[C@@H](C)N(C)CC4=O)c3)O[C@@](C)(C(F)(F)F)[C@H]2C)ccc(F)c1F]

(2R,3S,4S,5R)-3-(3,4-difluoro-2-
methoxyphenyl)-N-(2-((R)-4,5-dimethyl-2-
oxopiperazin-1-yl)pyridin-4-yl)-4,5-dimethyl-5-
(trifluoromethyl)tetrahydrofuran-2-carboxamide

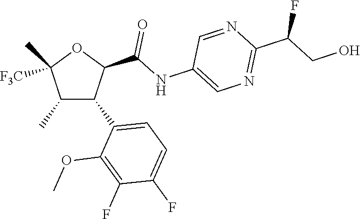 [SMILES: COc1c([C@H]2[C@H](C(=O)Nc3cnc([C@@H](F)CO)nc3)O[C@@](C)(C(F)(F)F)[C@H]2C)ccc(F)c1F]

(2R,3S,4S,5R)-3-(3,4-difluoro-2-methoxyphenyl)-N-(2-
((R)-1-fluoro-2-hydroxyethyl)pyrimidin-5-yl)-4,5-
dimethyl-5-(trifluoromethyl)tetrahydrofuran-2-
carboxamide

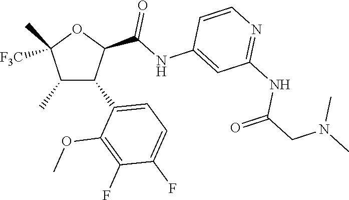 [SMILES: COc1c([C@H]2[C@H](C(=O)Nc3ccnc(NC(=O)CN(C)C)c3)O[C@@](C)(C(F)(F)F)[C@H]2C)ccc(F)c1F]

(2R,3S,4S,5R)-3-(3,4-difluoro-2-methoxyphenyl)-N-
(2-(2-(dimethylamino)acetamido)pyridin-4-yl)-4,5-
dimethyl-5-(trifluoromethyl)tetrahydrofuran-2-
carboxamide

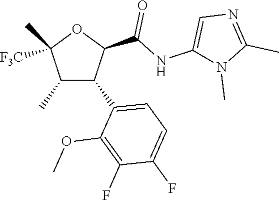 [SMILES: COc1c([C@H]2[C@H](C(=O)Nc3cnc(C)n3C)O[C@@](C)(C(F)(F)F)[C@H]2C)ccc(F)c1F]

(2R,3S,4S,5R)-3-(3,4-difluoro-2-methoxyphenyl)-N-
(1,2-dimethyl-1H-imidazol-5-yl)-4,5-dimethyl-5-
(trifluoromethyl)tetrahydrofuran-2-carboxamide

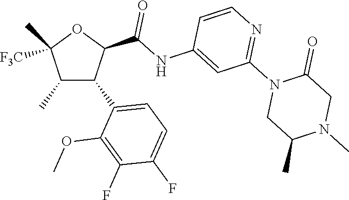 [SMILES: COc1c([C@H]2[C@H](C(=O)Nc3ccnc(N4C[C@H](C)N(C)CC4=O)c3)O[C@@](C)(C(F)(F)F)[C@H]2C)ccc(F)c1F]

(2R,3S,4S,5R)-3-(3,4-difluoro-2-
methoxyphenyl)-N-(2-((S)-4,5-dimethyl-2-
oxopiperazin-1-yl)pyridin-4-yl)-4,5-dimethyl-5-
(trifluoromethyl)tetrahydrofuran-2-carboxamide

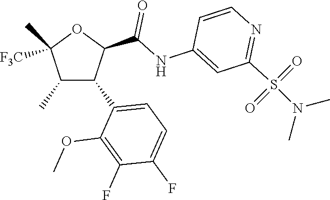 [SMILES: COc1c([C@H]2[C@H](C(=O)Nc3ccnc(S(=O)(=O)N(C)C)c3)O[C@@](C)(C(F)(F)F)[C@H]2C)ccc(F)c1F]

(2R,3S,4S,5R)-3-(3,4-difluoro-2-methoxyphenyl)-N-(2-
(N,N-dimethylsulfamoyl)pyridin-4-yl)-4,5-dimethyl-5-
(trifluoromethyl)tetrahydrofuran-2-carboxamide

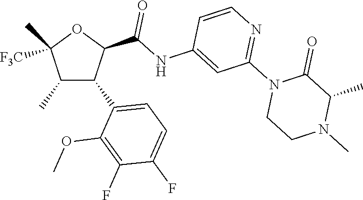 [SMILES: COc1c([C@H]2[C@H](C(=O)Nc3ccnc(N4CCN(C)[C@@H](C)C4=O)c3)O[C@@](C)(C(F)(F)F)[C@H]2C)ccc(F)c1F]

(2R,3S,4S,5R)-3-(3,4-difluoro-2-
methoxyphenyl)-N-(2-((S)-3,4-dimethyl-2-
oxopiperazin-1-yl)pyridin-4-yl)-4,5-dimethyl-5-
(trifluoromethyl)tetrahydrofuran-2-carboxamide

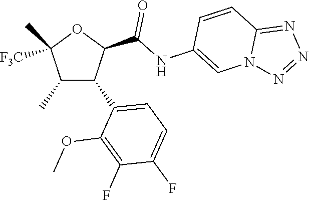 [SMILES: COc1c([C@H]2[C@H](C(=O)Nc3ccc4nnnn4c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(tetrazolo[1,5-a]pyridin-6-yl)-5-
(trifluoromethyl)tetrahydrofuran-2-carboxamide

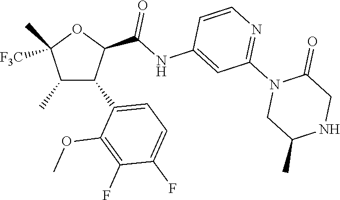 [SMILES: COc1c([C@H]2[C@H](C(=O)Nc3ccnc(N4C[C@H](C)NCC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-((S)-5-
methyl-2-oxopiperazin-1-yl)pyridin-4-yl)-5-
(trifluoromethyl)tetrahydrofuran-2-carboxamide

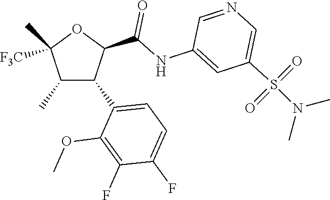 [SMILES: COc1c([C@H]2[C@H](C(=O)Nc3cncc(S(=O)(=O)N(C)C)c3)O[C@@](C)(C(F)(F)F)[C@H]2C)ccc(F)c1F]

(2R,3S,4S,5R)-3-(3,4-difluoro-2-methoxyphenyl)-N-(5-
(N,N-dimethylsulfamoyl)pyridin-3-yl)-4,5-dimethyl-5-
(trifluoromethyl)tetrahydrofuran-2-carboxamide

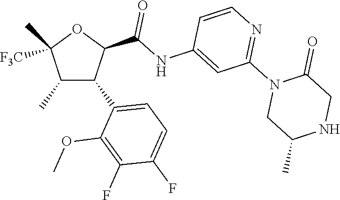 [SMILES: COc1c([C@H]2[C@H](C(=O)Nc3ccnc(N4C[C@@H](C)NCC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-((R)-5-
methyl-2-oxopiperazin-1-yl)pyridin-4-yl)-5-
(trifluoromethyl)tetrahydrofuran-2-carboxamide

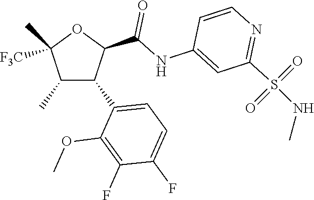 [SMILES: CNS(=O)(=O)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-
methoxyphenyl)-4,5-dimethyl-N-(2-(N-
methylsulfamoyl)pyridin-4-yl)-5-
(trifluoromethyl)tetrahydrofuran-2-carboxamide

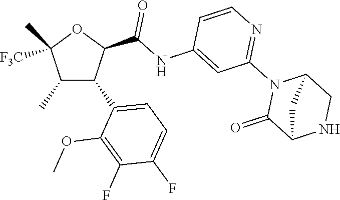 [SMILES: COc1c([C@H]2[C@H](C(=O)Nc3ccnc(N4C(=O)[C@H]5C[C@@H]4CN5)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1R,4R)-3-oxo-2,5-
diazabicyclo[2.2.1]heptan-2-yl)pyridin-4-yl)-5-
(trifluoromethyl)tetrahydrofuran-2-carboxamide

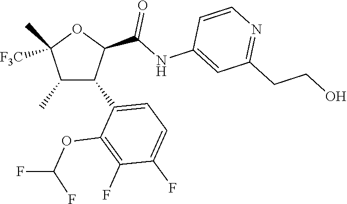 [SMILES: C[C@H]1[C@@H](c2ccc(F)c(F)c2OC(F)F)[C@H](C(=O)Nc2ccnc(CCO)c2)O[C@@]1(C)C(F)(F)F]

(2R,3S,4S,5R)-3-(2-(difluoromethoxy)-3,4-
difluorophenyl)-N-(2-(2-hydroxyethyl)pyridin-4-yl)-4,5-
dimethyl-5-(trifluoromethyl)tetrahydrofuran-2-
carboxamide

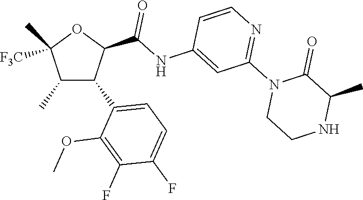 [SMILES: COc1c([C@H]2[C@H](C(=O)Nc3ccnc(N4CCN[C@H](C)C4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-((R)-3-
methyl-2-oxopiperazin-1-yl)pyridin-4-yl)-5-
(trifluoromethyl)tetrahydrofuran-2-carboxamide

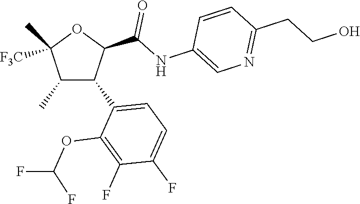 [SMILES: C[C@H]1[C@@H](c2ccc(F)c(F)c2OC(F)F)[C@H](C(=O)Nc2ccc(CCO)nc2)O[C@@]1(C)C(F)(F)F]

(2R,3S,4S,5R)-3-(2-(difluoromethoxy)-3,4-
difluorophenyl)-N-(6-(2-hydroxyethyl)pyridin-3-yl)-4,5-
dimethyl-5-(trifluoromethyl)tetrahydrofuran-2-
carboxamide

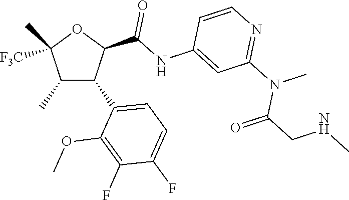 [SMILES: CNCC(=O)N(C)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
4,5-dimethyl-N-(2-(N-methyl-2-
(methylamino)acetamido)pyridin-4-yl)-5-
(trifluoromethyl)tetrahydrofuran-2-carboxamide

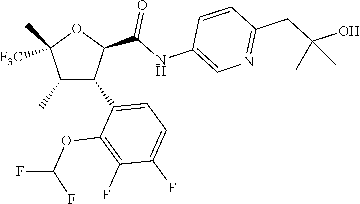 [SMILES: C[C@H]1[C@@H](c2ccc(F)c(F)c2OC(F)F)[C@H](C(=O)Nc2ccc(CC(C)(C)O)nc2)O[C@@]1(C)C(F)(F)F]

(2R,3S,4S,5R)-3-(2-(difluoromethoxy)-3,4-
difluorophenyl)-N-(6-(2-hydroxy-2-methylpropyl)pyridin-
3-yl)-4,5-dimethyl-5-(trifluoromethyl)tetrahydrofuran-2-
carboxamide

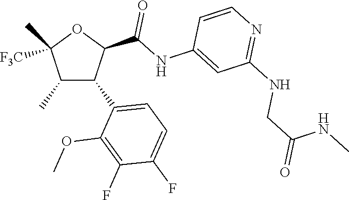 [SMILES: CNC(=O)CNc1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
4,5-dimethyl-N-(2-((2-(methylamino)-2-
oxoethyl)amino)pyridin-4-yl)-5-
(trifluoromethyl)tetrahydrofuran-2-carboxamide

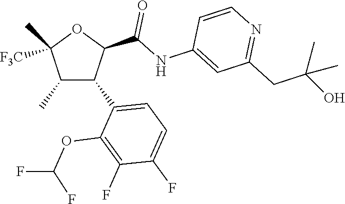 [SMILES: C[C@H]1[C@@H](c2ccc(F)c(F)c2OC(F)F)[C@H](C(=O)Nc2ccnc(CC(C)(C)O)c2)O[C@@]1(C)C(F)(F)F]

(2R,3S,4S,5R)-3-(2-(difluoromethoxy)-3,4-
difluorophenyl)-N-(2-(2-hydroxy-2-
methylpropyl)pyridin-4-yl)-4,5-dimethyl-5-
(trifluoromethyl)tetrahydrofuran-2-carboxamide

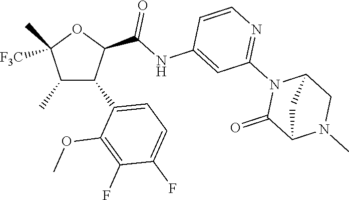 [SMILES: COc1c([C@H]2[C@H](C(=O)Nc3ccnc(N4C(=O)[C@H]5C[C@@H]4CN5C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1R,4R)-5-methyl-3-oxo-2,5-
diazabicyclo[2.2.1]heptan-2-yl)pyridin-4-yl)-5-
(trifluoromethyl)tetrahydrofuran-2-carboxamide

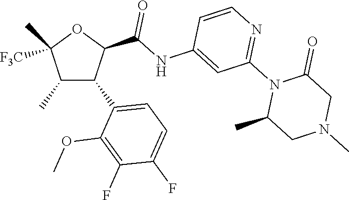 [SMILES: COc1c([C@H]2[C@H](C(=O)Nc3ccnc(N4C(=O)CN(C)C[C@H]4C)c3)O[C@@](C)(C(F)(F)F)[C@H]2C)ccc(F)c1F]

(2R,3S,4S,5R)-3-(3,4-difluoro-2-
methoxyphenyl)-N-(2-((R)-2,4-dimethyl-6-
oxopiperazin-1-yl)pyridin-4-yl)-4,5-dimethyl-5-
(trifluoromethyl)tetrahydrofuran-2-carboxamide

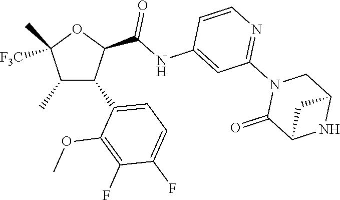 [SMILES: COc1c([C@H]2[C@H](C(=O)Nc3ccnc(N4C[C@@H]5C[C@@H](N5)C4=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1R,5S)-2-oxo-3,6-
diazabicyclo[3.1.1]heptan-3-yl)pyridin-4-yl)-5-
(trifluoromethyl)tetrahydrofuran-2-carboxamide

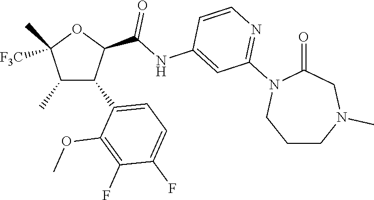 [SMILES: COc1c([C@H]2[C@H](C(=O)Nc3ccnc(N4CCCN(C)CC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(4-methyl-2-
oxo-1,4-diazepan-1-yl)pyridin-4-yl)-5-
(trifluoromethyl)tetrahydrofuran-2-carboxamide

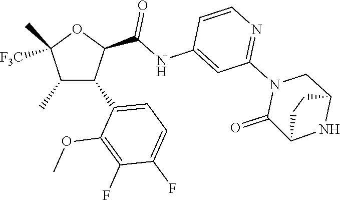 [SMILES: COc1c([C@H]2[C@H](C(=O)Nc3ccnc(N4C[C@@H]5CC[C@@H](N5)C4=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1R,5S)-2-oxo-3,8-
diazabicyclo[3.2.1]octan-3-yl)pyridin-4-yl)-5-
(trifluoromethyl)tetrahydrofuran-2-carboxamide

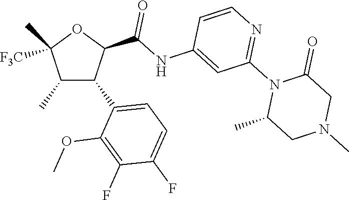 [SMILES: COc1c([C@H]2[C@H](C(=O)Nc3ccnc(N4C(=O)CN(C)C[C@@H]4C)c3)O[C@@](C)(C(F)(F)F)[C@H]2C)ccc(F)c1F]

(2R,3S,4S,5R)-3-(3,4-difluoro-2-
methoxyphenyl)-N-(2-((S)-2,4-dimethyl-6-
oxopiperazin-1-yl)pyridin-4-yl)-4,5-dimethyl-5-
(trifluoromethyl)tetrahydrofuran-2-carboxamide

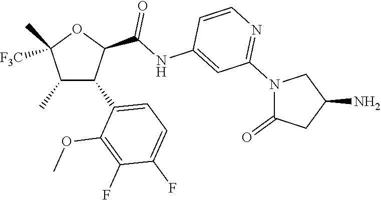 [SMILES: COc1c([C@H]2[C@H](C(=O)Nc3ccnc(N4C[C@@H](N)CC4=O)c3)O[C@@](C)(C(F)(F)F)[C@H]2C)ccc(F)c1F]

(2R,3S,4S,5R)-N-(2-((S)-4-amino-2-
oxopyrrolidin-1-yl)pyridin-4-yl)-3-(3,4-difluoro-2-
methoxyphenyl)-4,5-dimethyl-5-
(trifluoromethyl)tetrahydrofuran-2-carboxamide

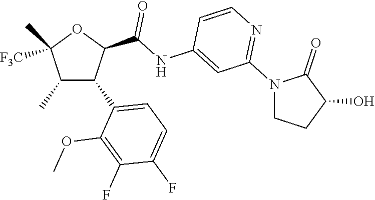 [SMILES: COc1c([C@H]2[C@H](C(=O)Nc3ccnc(N4CC[C@@H](O)C4=O)c3)O[C@@](C)(C(F)(F)F)[C@H]2C)ccc(F)c1F]

(2R,3S,4S,5R)-3-(3,4-difluoro-2-
methoxyphenyl)-N-(2-((R)-3-hydroxy-2-
oxopyrrolidin-1-yl)pyridin-4-yl)-4,5-dimethyl-5-
(trifluoromethyl)tetrahydrofuran-2-carboxamide

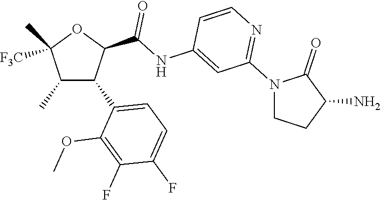 [SMILES: COc1c([C@H]2[C@H](C(=O)Nc3ccnc(N4CC[C@@H](N)C4=O)c3)O[C@@](C)(C(F)(F)F)[C@H]2C)ccc(F)c1F]

(2R,3S,4S,5R)-N-(2-((R)-3-amino-2-oxopyrrolidin-
1-yl)pyridin-4-yl)-3-(3,4-difluoro-2-methoxyphenyl)-
4,5-dimethyl-5-(trifluoromethyl)tetrahydrofuran-2-
carboxamide

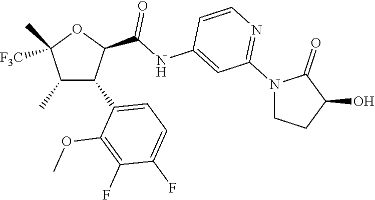 [SMILES: COc1c([C@H]2[C@H](C(=O)Nc3ccnc(N4CC[C@H](O)C4=O)c3)O[C@@](C)(C(F)(F)F)[C@H]2C)ccc(F)c1F]

(2R,3S,4S,5R)-3-(3,4-difluoro-2-
methoxyphenyl)-N-(2-((S)-3-hydroxy-2-
oxopyrrolidin-1-yl)pyridin-4-yl)-4,5-dimethyl-5-
(trifluoromethyl)tetrahydrofuran-2-carboxamide

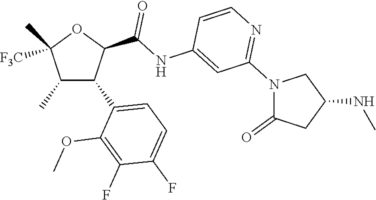 [SMILES: CN[C@@H]1CC(=O)N(c2cc(NC(=O)[C@@H]3O[C@@](C)(C(F)(F)F)[C@@H](C)[C@H]3c3ccc(F)c(F)c3OC)ccn2)C1]

(2R,3S,4S,5R)-3-(3,4-difluoro-2-methoxyphenyl)-
4,5-dimethyl-N-(2-((R)-4-(methylamino)-2-
oxopyrrolidin-1-yl)pyridin-4-yl)-5-
(trifluoromethyl)tetrahydrofuran-2-carboxamide

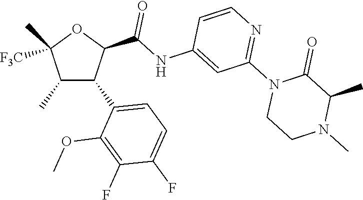 [SMILES: COc1c([C@H]2[C@H](C(=O)Nc3ccnc(N4CCN(C)[C@H](C)C4=O)c3)O[C@@](C)(C(F)(F)F)[C@H]2C)ccc(F)c1F]

(2R,3S,4S,5R)-3-(3,4-difluoro-2-
methoxyphenyl)-N-(2-((R)-3,4-dimethyl-2-
oxopiperazin-1-yl)pyridin-4-yl)-4,5-dimethyl-5-
(trifluoromethyl)tetrahydrofuran-2-carboxamide

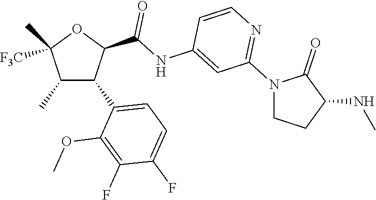 [SMILES: CN[C@@H]1CCN(c2cc(NC(=O)[C@@H]3O[C@@](C)(C(F)(F)F)[C@@H](C)[C@H]3c3ccc(F)c(F)c3OC)ccn2)C1=O]

(2R,3S,4S,5R)-3-(3,4-difluoro-2-methoxyphenyl)-
4,5-dimethyl-N-(2-((R)-3-(methylamino)-2-
oxopyrrolidin-1-yl)pyridin-4-yl)-5-
(trifluoromethyl)tetrahydrofuran-2-carboxamide

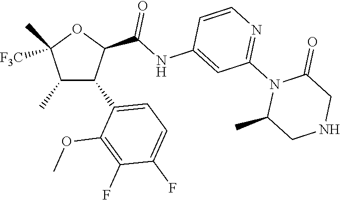 [SMILES: COc1c([C@H]2[C@H](C(=O)Nc3ccnc(N4C(=O)CNC[C@H]4C)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-((R)-2-
methyl-6-oxopiperazin-1-yl)pyridin-4-yl)-5-
(trifluoromethyl)tetrahydrofuran-2-carboxamide

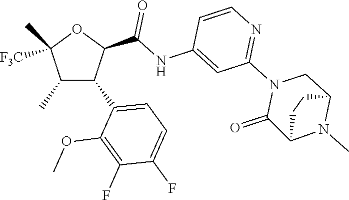 [SMILES: COc1c([C@H]2[C@H](C(=O)Nc3ccnc(N4C[C@@H]5CC[C@H](C4=O)N5C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1R,5S)-8-methyl-2-oxo-3,8-
diazabicyclo[3.2.1]octan-3-yl)pyridin-4-yl)-5-
(trifluoromethyl)tetrahydrofuran-2-carboxamide

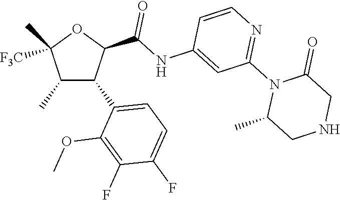 [SMILES: COc1c([C@H]2[C@H](C(=O)Nc3ccnc(N4C(=O)CNC[C@@H]4C)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-((S)-2-
methyl-6-oxopiperazin-1-yl)pyridin-4-yl)-5-
(trifluoromethyl)tetrahydrofuran-2-carboxamide

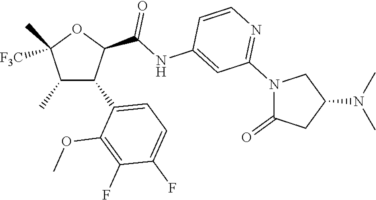 [SMILES: COc1c([C@H]2[C@H](C(=O)Nc3ccnc(N4C[C@H](N(C)C)CC4=O)c3)O[C@@](C)(C(F)(F)F)[C@H]2C)ccc(F)c1F]

(2R,3S,4S,5R)-3-(3,4-difluoro-2-methoxyphenyl)-N-
(2-((R)-4-(dimethylamino)-2-oxopyrrolidin-1-
yl)pyridin-4-yl)-4,5-dimethyl-5-
(trifluoromethyl)tetrahydrofuran-2-carboxamide

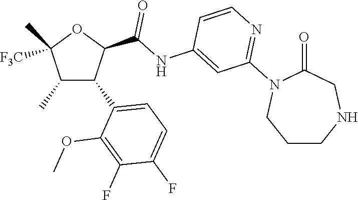 [SMILES: COc1c([C@H]2[C@H](C(=O)Nc3ccnc(N4CCCNCC4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-(2-oxo-1,4-
diazepan-1-yl)pyridin-4-yl)-5-
(trifluoromethyl)tetrahydrofuran-2-carboxamide

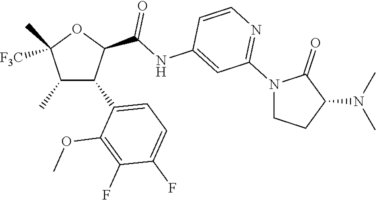 [SMILES: COc1c([C@H]2[C@H](C(=O)Nc3ccnc(N4CC[C@@H](N(C)C)C4=O)c3)O[C@@](C)(C(F)(F)F)[C@H]2C)ccc(F)c1F]

(2R,3S,4S,5R)-3-(3,4-difluoro-2-methoxyphenyl)-N-
(2-((R)-3-(dimethylamino)-2-oxopyrrolidin-1-
yl)pyridin-4-yl)-4,5-dimethyl-5-
(trifluoromethyl)tetrahydrofuran-2-carboxamide

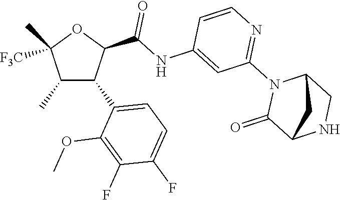 [SMILES: COc1c([C@H]2[C@H](C(=O)Nc3ccnc(N4C(=O)[C@@H]5C[C@H]4CN5)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1S,4S)-3-oxo-2,5-
diazabicyclo[2.2.1]heptan-2-yl)pyridin-4-yl)-5-
(trifluoromethyl)tetrahydrofuran-2-carboxamide

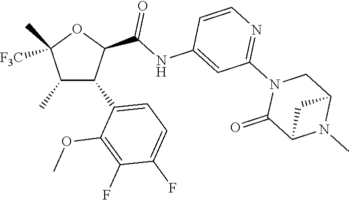 [SMILES: COc1c([C@H]2[C@H](C(=O)Nc3ccnc(N4C[C@@H]5C[C@H](C4=O)N5C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1R,5S)-6-methyl-2-oxo-3,6-
diazabicyclo[3.1.1]heptan-3-yl)pyridin-4-yl)-5-
(trifluoromethyl)tetrahydrofuran-2-carboxamide

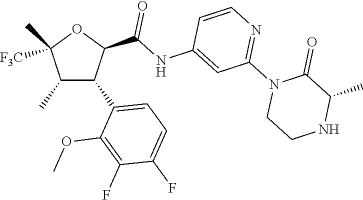 [SMILES: COc1c([C@H]2[C@H](C(=O)Nc3ccnc(N4CCN[C@@H](C)C4=O)c3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(2-((S)-3-
methyl-2-oxopiperazin-1-yl)pyridin-4-yl)-5-
(trifluoromethyl)tetrahydrofuran-2-carboxamide

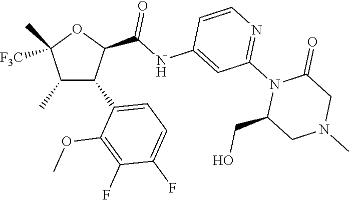 [SMILES: COc1c([C@H]2[C@H](C(=O)Nc3ccnc(N4C(=O)CN(C)C[C@H]4CO)c3)O[C@@](C)(C(F)(F)F)[C@H]2C)ccc(F)c1F]

(2R,3S,4S,5R)-3-(3,4-difluoro-2-methoxyphenyl)-
N-(2-((S)-2-(hydroxymethyl)-4-methyl-6-
oxopiperazin-1-yl)pyridin-4-yl)-4,5-dimethyl-5-
(trifluoromethyl)tetrahydrofuran-2-carboxamide

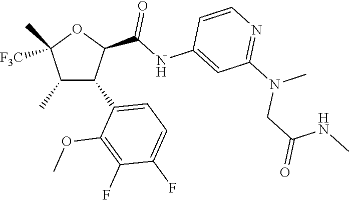 [SMILES: CNC(=O)CN(C)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
4,5-dimethyl-N-(2-(methyl(2-(methylamino)-2-
oxoethyl)amino)pyridin-4-yl)-5-
(trifluoromethyl)tetrahydrofuran-2-carboxamide

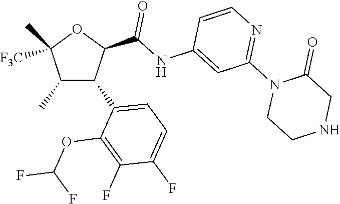 [SMILES: C[C@H]1[C@@H](c2ccc(F)c(F)c2OC(F)F)[C@H](C(=O)Nc2ccnc(N3CCNCC3=O)c2)O[C@@]1(C)C(F)(F)F]

(2R,3S,4S,5R)-3-(2-(difluoromethoxy)-3,4-
difluorophenyl)-4,5-dimethyl-N-(2-(2-
oxopiperazin-1-yl)pyridin-4-yl)-5-
(trifluoromethyl)tetrahydrofuran-2-carboxamide

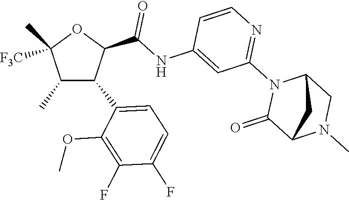 [SMILES: COc1c([C@H]2[C@H](C(=O)Nc3ccnc(N4C(=O)[C@@H]5C[C@H]4CN5C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1S,4S)-5-methyl-3-oxo-2,5-
diazabicyclo[2.2.1]heptan-2-yl)pyridin-4-yl)-5-
(trifluoromethyl)tetrahydrofuran-2-carboxamide

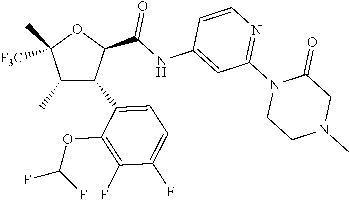 [SMILES: C[C@H]1[C@@H](c2ccc(F)c(F)c2OC(F)F)[C@H](C(=O)Nc2ccnc(N3CCN(C)CC3=O)c2)O[C@@]1(C)C(F)(F)F]

(2R,3S,4S,5R)-3-(2-(difluoromethoxy)-3,4-
difluorophenyl)-4,5-dimethyl-N-(2-(4-methyl-2-
oxopiperazin-1-yl)pyridin-4-yl)-5-
(trifluoromethyl)tetrahydrofuran-2-carboxamide

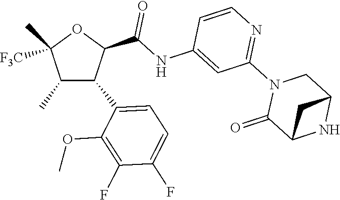 [SMILES: COc1c([C@H]2[C@H](C(=O)Nc3ccnc(N4C[C@H]5C[C@H](N5)C4=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1S,5R)-2-oxo-3,6-
diazabicyclo[3.1.1]heptan-3-yl)pyridin-4-yl)-5-
(trifluoromethyl)tetrahydrofuran-2-carboxamide

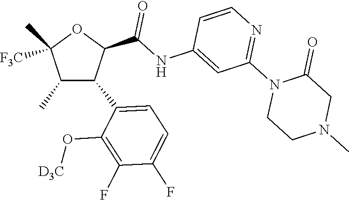 [SMILES: [2H]C([2H])([2H])Oc1c([C@H]2[C@H](C(=O)Nc3ccnc(N4CCN(C)CC4=O)c3)O[C@@](C)(C(F)(F)F)[C@H]2C)ccc(F)c1F]

(2R,3S,4S,5R)-3-(3,4-difluoro-2-(methoxy-
d₃)phenyl)-4,5-dimethyl-N-(2-(4-methyl-2-
oxopiperazin-1-yl)pyridin-4-yl)-5-
(trifluoromethyl)tetrahydrofuran-2-carboxamide

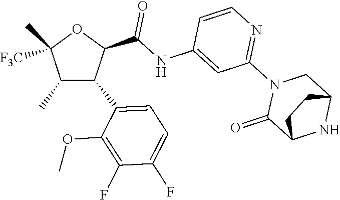 [SMILES: COc1c([C@H]2[C@H](C(=O)Nc3ccnc(N4C[C@H]5CC[C@H](N5)C4=O)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1S,5R)-2-oxo-3,8-
diazabicyclo[3.2.1]octan-3-yl)pyridin-4-yl)-5-
(trifluoromethyl)tetrahydrofuran-2-carboxamide

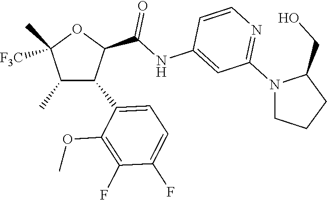 [SMILES: COc1c([C@H]2[C@H](C(=O)Nc3ccnc(N4CCC[C@@H]4CO)c3)O[C@@](C)(C(F)(F)F)[C@H]2C)ccc(F)c1F]

(2R,3S,4S,5R)-3-(3,4-difluoro-2-methoxyphenyl)-
N-(2-((R)-2-(hydroxymethyl)pyrrolidin-1-yl)pyridin-
4-yl)-4,5-dimethyl-5-
(trifluoromethyl)tetrahydrofuran-2-carboxamide

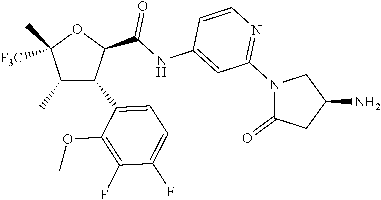 [SMILES: COc1c([C@H]2[C@H](C(=O)Nc3ccnc(N4C[C@@H](N)CC4=O)c3)O[C@@](C)(C(F)(F)F)[C@H]2C)ccc(F)c1F]

(2R,3S,4S,5R)-N-(2-((S)-4-amino-2-
oxopyrrolidin-1-yl)pyridin-4-yl)-3-(3,4-difluoro-2-
methoxyphenyl)-4,5-dimethyl-5-
(trifluoromethyl)tetrahydrofuran-2-carboxamide

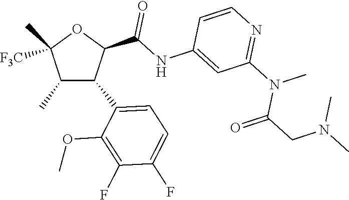 [SMILES: COc1c([C@H]2[C@H](C(=O)Nc3ccnc(N(C)C(=O)CN(C)C)c3)O[C@@](C)(C(F)(F)F)[C@H]2C)ccc(F)c1F]

(2R,3S,4S,5R)-3-(3,4-difluoro-2-
methoxyphenyl)-N-(2-(2-(dimethylamino)-N-
methylacetamido)pyridin-4-yl)-4,5-dimethyl-5-
(trifluoromethyl)tetrahydrofuran-2-carboxamide

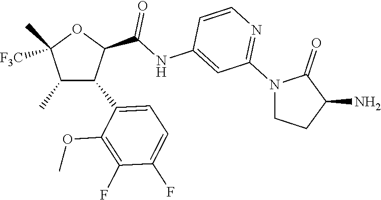 [SMILES: COc1c([C@H]2[C@H](C(=O)Nc3ccnc(N4CC[C@H](N)C4=O)c3)O[C@@](C)(C(F)(F)F)[C@H]2C)ccc(F)c1F]

(2R,3S,4S,5R)-N-(2-((S)-3-amino-2-
oxopyrrolidin-1-yl)pyridin-4-yl)-3-(3,4-difluoro-2-
methoxyphenyl)-4,5-dimethyl-5-
(trifluoromethyl)tetrahydrofuran-2-carboxamide

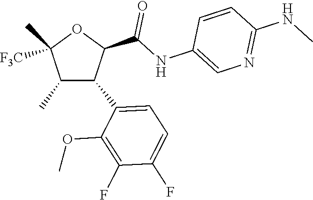 [SMILES: CNc1ccc(NC(=O)[C@@H]2O[C@@](C)(C(F)(F)F)[C@@H](C)[C@H]2c2ccc(F)c(F)c2OC)cn1]

(2R,3S,4S,5R)-3-(3,4-difluoro-2-
methoxyphenyl)-4,5-dimethyl-N-(6-
(methylamino)pyridin-3-yl)-5-
(trifluoromethyl)tetrahydrofuran-2-carboxamide

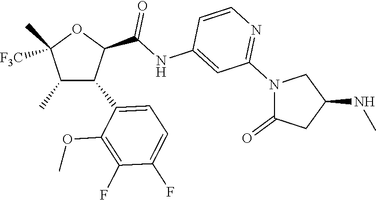 [SMILES: CN[C@H]1CC(=O)N(c2cc(NC(=O)[C@@H]3O[C@@](C)(C(F)(F)F)[C@@H](C)[C@H]3c3ccc(F)c(F)c3OC)ccn2)C1]

(2R,3S,4S,5R)-3-(3,4-difluoro-2-methoxyphenyl)-
4,5-dimethyl-N-(2-((S)-4-(methylamino)-2-
oxopyrrolidin-1-yl)pyridin-4-yl)-5-
(trifluoromethyl)tetrahydrofuran-2-carboxamide

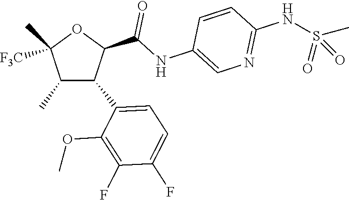 [SMILES: COc1c([C@H]2[C@H](C(=O)Nc3ccc(NS(C)(=O)=O)nc3)O[C@@](C)(C(F)(F)F)[C@H]2C)ccc(F)c1F]

(2R,3S,4S,5R)-3-(3,4-difluoro-2-
methoxyphenyl)-4,5-dimethyl-N-(6-
(methylsulfonamido)pyridin-3-yl)-5-
(trifluoromethyl)tetrahydrofuran-2-carboxamide

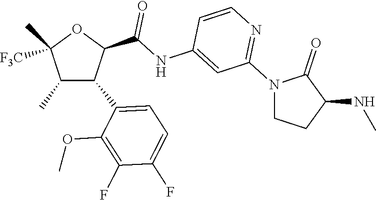 [SMILES: CN[C@H]1CCN(c2cc(NC(=O)[C@@H]3O[C@@](C)(C(F)(F)F)[C@@H](C)[C@H]3c3ccc(F)c(F)c3OC)ccn2)C1=O]

(2R,3S,4S,5R)-3-(3,4-difluoro-2-methoxyphenyl)-
4,5-dimethyl-N-(2-((S)-3-(methylamino)-2-
oxopyrrolidin-1-yl)pyridin-4-yl)-5-
(trifluoromethyl)tetrahydrofuran-2-carboxamide

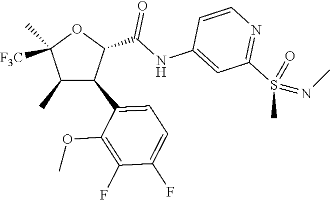 [SMILES: CN=[S@@](C)(=O)c1cc(NC(=O)[C@H]2O[C@](C)(C(F)(F)F)[C@H](C)[C@@H]2c2ccc(F)c(F)c2OC)ccn1]

(2S,3R,4R,5S)-3-(3,4-difluoro-2-methoxyphenyl)-
N-(2-(N,S-dimethylsulfonimidoyl)pyridin-4-yl)-4,5-
dimethyl-5-(trifluoromethyl)tetrahydrofuran-2-
carboxamide

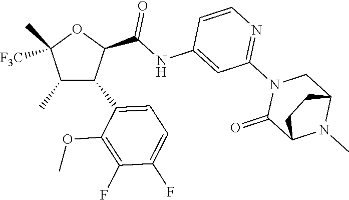 [SMILES: COc1c([C@H]2[C@H](C(=O)Nc3ccnc(N4C[C@H]5CC[C@@H](C4=O)N5C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1S,5R)-8-methyl-2-oxo-3,8-
diazabicyclo[3.2.1]octan-3-yl)pyridin-4-yl)-5-
(trifluoromethyl)tetrahydrofuran-2-carboxamide

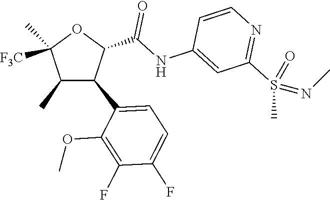 [SMILES: CN=[S@](C)(=O)c1cc(NC(=O)[C@H]2O[C@](C)(C(F)(F)F)[C@H](C)[C@@H]2c2ccc(F)c(F)c2OC)ccn1]

(2S,3R,4R,5S)-3-(3,4-difluoro-2-methoxyphenyl)-
N-(2-(N,S-dimethylsulfonimidoyl)pyridin-4-yl)-4,5-
dimethyl-5-(trifluoromethyl)tetrahydrofuran-2-
carboxamide

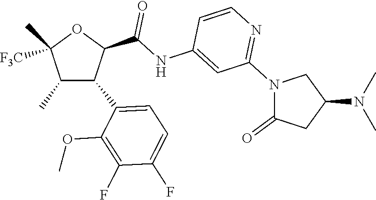 [SMILES: COc1c([C@H]2[C@H](C(=O)Nc3ccnc(N4C[C@@H](N(C)C)CC4=O)c3)O[C@@](C)(C(F)(F)F)[C@H]2C)ccc(F)c1F]

(2R,3S,4S,5R)-3-(3,4-difluoro-2-methoxyphenyl)-N-
(2-((S)-4-(dimethylamino)-2-oxopyrrolidin-1-
yl)pyridin-4-yl)-4,5-dimethyl-5-
(trifluoromethyl)tetrahydrofuran-2-carboxamide

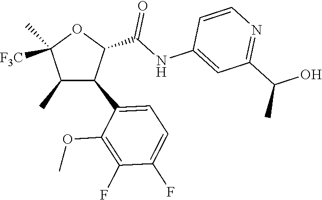 [SMILES: COc1c([C@@H]2[C@@H](C(=O)Nc3ccnc([C@H](C)O)c3)O[C@](C)(C(F)(F)F)[C@@H]2C)ccc(F)c1F]

(2S,3R,4R,5S)-3-(3,4-difluoro-2-methoxyphenyl)-
N-(2-((S)-1-hydroxyethyl)pyridin-4-yl)-4,5-
dimethyl-5-(trifluoromethyl)tetrahydrofuran-2-
carboxamide

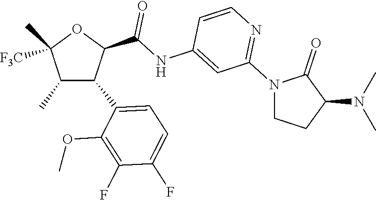 [SMILES: COc1c([C@H]2[C@H](C(=O)Nc3ccnc(N4CC[C@H](N(C)C)C4=O)c3)O[C@@](C)(C(F)(F)F)[C@H]2C)ccc(F)c1F]

(2R,3S,4S,5R)-3-(3,4-difluoro-2-methoxyphenyl)-N-
(2-((S)-3-(dimethylamino)-2-oxopyrrolidin-1-
yl)pyridin-4-yl)-4,5-dimethyl-5-
(trifluoromethyl)tetrahydrofuran-2-carboxamide

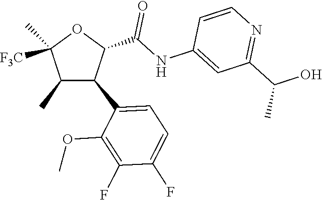 [SMILES: COc1c([C@@H]2[C@@H](C(=O)Nc3ccnc([C@@H](C)O)c3)O[C@](C)(C(F)(F)F)[C@@H]2C)ccc(F)c1F]

(2S,3R,4R,5S)-3-(3,4-difluoro-2-methoxyphenyl)-
N-(2-((R)-1-hydroxyethyl)pyridin-4-yl)-4,5-
dimethyl-5-(trifluoromethyl)tetrahydrofuran-2-
carboxamide

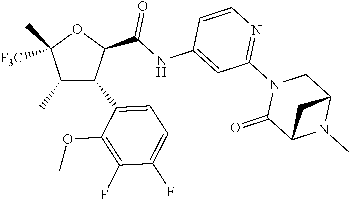 [SMILES: COc1c([C@H]2[C@H](C(=O)Nc3ccnc(N4C[C@H]5C[C@@H](C4=O)N5C)c3)O[C@@](C)(C(F)(F)F)[C@H]2C)ccc(F)c1F]

(2R,3S,4S,5R)-3-(3,4-difluoro-2-methoxyphenyl)-
4,5-dimethyl-N-(2-((1S,5R)-6-methyl-2-oxo-3,6-
diazabicyclo[3.1.1]heptan-3-yl)pyridin-4-yl)-5-
(trifluoromethyl)tetrahydrofuran-2-carboxamide

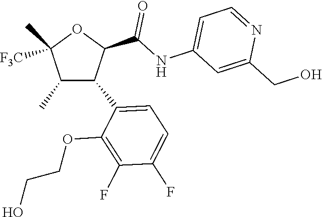 [SMILES: C[C@H]1[C@@H](c2ccc(F)c(F)c2OCCO)[C@H](C(=O)Nc2ccnc(CO)c2)O[C@@]1(C)C(F)(F)F]

(2R,3S,4S,5R)-3-(3,4-difluoro-2-(2-
hydroxyethoxy)phenyl)-N-(2-
(hydroxymethyl)pyridin-4-yl)-4,5-dimethyl-5-
(trifluoromethyl)tetrahydrofuran-2-carboxamide

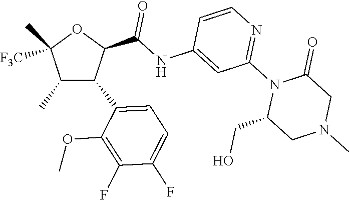 [SMILES: COc1c([C@H]2[C@H](C(=O)Nc3ccnc(N4C(=O)CN(C)C[C@@H]4CO)c3)O[C@@](C)(C(F)(F)F)[C@H]2C)ccc(F)c1F]

(2R,3S,4S,5R)-3-(3,4-difluoro-2-methoxyphenyl)-
N-(2-((R)-2-(hydroxymethyl)-4-methyl-6-
oxopiperazin-1-yl)pyridin-4-yl)-4,5-dimethyl-5-
(trifluoromethyl)tetrahydrofuran-2-carboxamide

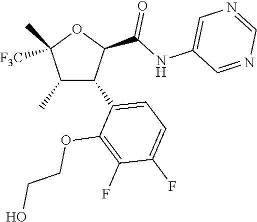 [SMILES: C[C@H]1[C@@H](c2ccc(F)c(F)c2OCCO)[C@H](C(=O)Nc2cncnc2)O[C@@]1(C)C(F)(F)F]

(2R,3S,4S,5R)-3-(3,4-difluoro-2-(2-
hydroxyethoxy)phenyl)-4,5-dimethyl-N-(pyrimidin-
5-yl)-5-(trifluoromethyl)tetrahydrofuran-2-
carboxamide

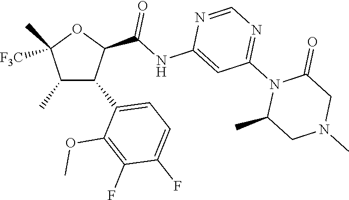 [SMILES: COc1c([C@H]2[C@H](C(=O)Nc3cc(N4C(=O)CN(C)C[C@H]4C)ncn3)O[C@@](C)(C(F)(F)F)[C@H]2C)ccc(F)c1F]

(2R,3S,4S,5R)-3-(3,4-difluoro-2-methoxyphenyl)-
N-(6-((R)-2,4-dimethyl-6-oxopiperazin-1-
yl)pyrimidin-4-yl)-4,5-dimethyl-5-
(trifluoromethyl)tetrahydrofuran-2-carboxamide

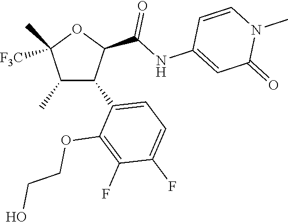 [SMILES: C[C@H]1[C@@H](c2ccc(F)c(F)c2OCCO)[C@H](C(=O)Nc2ccn(C)c(=O)c2)O[C@@]1(C)C(F)(F)F]

(2R,3S,4S,5R)-3-(3,4-difluoro-2-(2-
hydroxyethoxy)phenyl)-4,5-dimethyl-N-(1-methyl-
2-oxo-1,2-dihydropyridin-4-yl)-5-
(trifluoromethyl)tetrahydrofuran-2-carboxamide

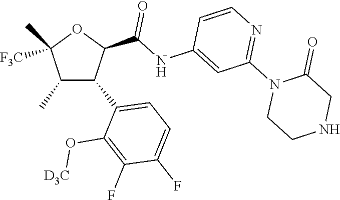 [SMILES: [2H]C([2H])([2H])Oc1c([C@H]2[C@H](C(=O)Nc3ccnc(N4CCNCC4=O)c3)O[C@@](C)(C(F)(F)F)[C@H]2C)ccc(F)c1F]

(2R,3S,4S,5R)-3-(3,4-difluoro-2-(methoxy-
d₃)phenyl)-4,5-dimethyl-N-(2-(2-oxopiperazin-1-
yl)pyridin-4-yl)-5-(trifluoromethyl)tetrahydrofuran-
2-carboxamide

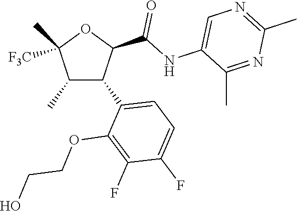 [SMILES: Cc1ncc(NC(=O)[C@@H]2O[C@@](C)(C(F)(F)F)[C@@H](C)[C@H]2c2ccc(F)c(F)c2OCCO)c(C)n1]

(2R,3S,4S,5R)-3-(3,4-difluoro-2-(2-
hydroxyethoxy)phenyl)-N-(2,4-
dimethylpyrimidin-5-yl)-4,5-dimethyl-5-
(trifluoromethyl)tetrahydrofuran-2-carboxamide

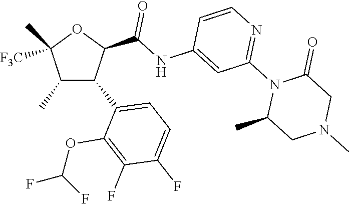 [SMILES: C[C@@H]1CN(C)CC(=O)N1c1cc(NC(=O)[C@@H]2O[C@@](C)(C(F)(F)F)[C@@H](C)[C@H]2c2ccc(F)c(F)c2OC(F)F)ccn1]

(2R,3S,4S,5R)-3-(2-(difluoromethoxy)-3,4-
difluorophenyl)-N-(2-((R)-2,4-dimethyl-6-
oxopiperazin-1-yl)pyridin-4-yl)-4,5-dimethyl-5-
(trifluoromethyl)tetrahydrofuran-2-carboxamide

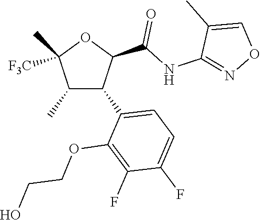 [SMILES: Cc1conc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-4,5-dimethyl-N-(4-
methylisoxazol-3-yl)-5-
(trifluoromethyl)tetrahydrofuran-2-carboxamide

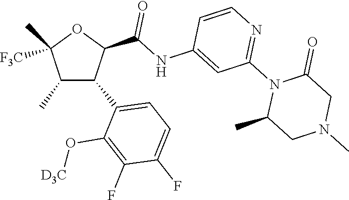 [SMILES: [2H]C([2H])([2H])Oc1c([C@H]2[C@H](C(=O)Nc3ccnc(N4C(=O)CN(C)C[C@H]4C)c3)O[C@@](C)(C(F)(F)F)[C@H]2C)ccc(F)c1F]

(2R,3S,4S,5R)-3-(3,4-difluoro-2-(methoxy-
d₃)phenyl)-N-(2-((R)-2,4-dimethyl-6-oxopiperazin-
1-yl)pyridin-4-yl)-4,5-dimethyl-5-
(trifluoromethyl)tetrahydrofuran-2-carboxamide

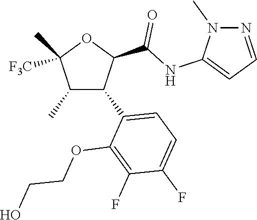 [SMILES: C[C@H]1[C@@H](c2ccc(F)c(F)c2OCCO)[C@H](C(=O)Nc2ccnn2C)O[C@@]1(C)C(F)(F)F]

(2R,3S,4S,5R)-3-(3,4-difluoro-2-(2-
hydroxyethoxy)phenyl)-4,5-dimethyl-N-(1-methyl-
1H-pyrazol-5-yl)-5-
(trifluoromethyl)tetrahydrofuran-2-carboxamide

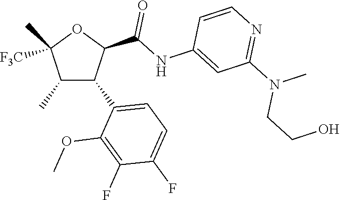 [SMILES: COc1c([C@H]2[C@H](C(=O)Nc3ccnc(N(C)CCO)c3)O[C@@](C)(C(F)(F)F)[C@H]2C)ccc(F)c1F]

(2R,3S,4S,5R)-3-(3,4-difluoro-2-
methoxyphenyl)-N-(2-((2-
hydroxyethyl)(methyl)amino)pyridin-4-yl)-4,5-
dimethyl-5-(trifluoromethyl)tetrahydrofuran-2-
carboxamide

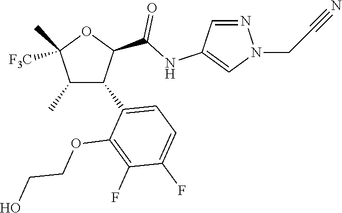 [SMILES: C[C@H]1[C@@H](c2ccc(F)c(F)c2OCCO)[C@H](C(=O)Nc2cnn(CC#N)c2)O[C@@]1(C)C(F)(F)F]

(2R,3S,4S,5R)-N-(1-(cyanomethyl)-1H-pyrazol-
4-yl)-3-(3,4-difluoro-2-(2-hydroxyethoxy)phenyl)-
4,5-dimethyl-5-(trifluoromethyl)tetrahydrofuran-
2-carboxamide

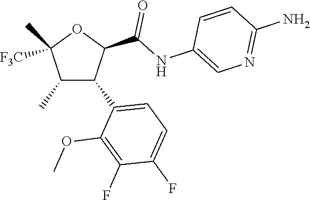 [SMILES: COc1c([C@H]2[C@H](C(=O)Nc3ccc(N)nc3)O[C@@](C)(C(F)(F)F)[C@H]2C)ccc(F)c1F]

(2R,3S,4S,5R)-N-(6-aminopyridin-3-yl)-3-(3,4-
difluoro-2-methoxyphenyl)-4,5-dimethyl-5-
(trifluoromethyl)tetrahydrofuran-2-carboxamide

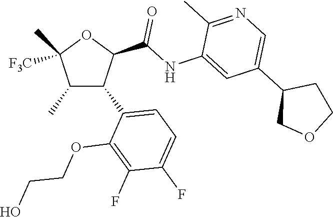 [SMILES: Cc1ncc([C@H]2CCOC2)cc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-4,5-dimethyl-N-(2-methyl-
5-((S)-tetrahydrofuran-3-yl)pyridin-3-yl)-5-
(trifluoromethyl)tetrahydrofuran-2-carboxamide

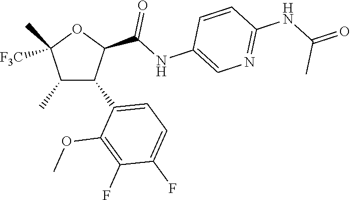 [SMILES: COc1c([C@H]2[C@H](C(=O)Nc3ccc(NC(C)=O)nc3)O[C@@](C)(C(F)(F)F)[C@H]2C)ccc(F)c1F]

(2R,3S,4S,5R)-N-(6-acetamidopyridin-3-yl)-3-
(3,4-difluoro-2-methoxyphenyl)-4,5-dimethyl-5-
(trifluoromethyl)tetrahydrofuran-2-carboxamide

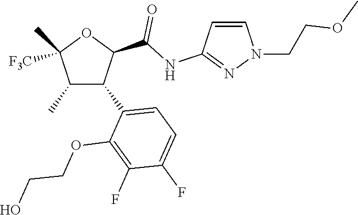 [SMILES: COCCn1ccc(NC(=O)[C@@H]2O[C@@](C)(C(F)(F)F)[C@@H](C)[C@H]2c2ccc(F)c(F)c2OCCO)n1]

(2R,3S,4S,5R)-3-(3,4-difluoro-2-(2-
hydroxyethoxy)phenyl)-N-(1-(2-methoxyethyl)-
1H-pyrazol-3-yl)-4,5-dimethyl-5-
(trifluoromethyl)tetrahydrofuran-2-carboxamide

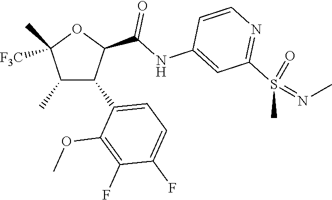 [SMILES: CN=[S@@](C)(=O)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-(N,S-dimethylsulfonimidoyl)pyridin-4-yl)-4,5-
dimethyl-5-(trifluoromethyl)tetrahydrofuran-2-
carboxamide

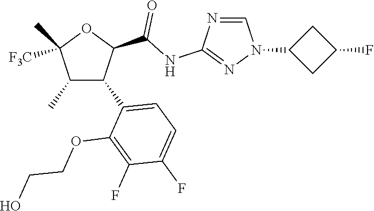

(2R,3S,4S,5R)-3-(3,4-difluoro-2-(2-
hydroxyethoxy)phenyl)-N-(1-((1s,3S)-3-
fluorocyclobutyl)-1H-1,2,4-triazol-3-yl)-4,5-dimethyl-5-
(trifluoromethyl)tetrahydrofuran-2-carboxamide

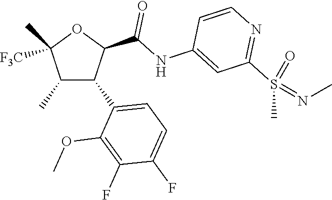 [SMILES: CN=[S@](C)(=O)c1cc(NC(=O)[C@@H]2O[C@@](C)(C(F)(F)F)[C@@H](C)[C@H]2c2ccc(F)c(F)c2OC)ccn1]

(2R,3S,4S,5R)-3-(3,4-difluoro-2-methoxyphenyl)-
N-(2-(N,S-dimethylsulfonimidoyl)pyridin-4-yl)-4,5-
dimethyl-5-(trifluoromethyl)tetrahydrofuran-2-
carboxamide

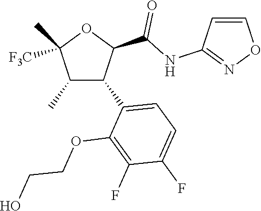 [SMILES: C[C@H]1[C@@H](c2ccc(F)c(F)c2OCCO)[C@H](C(=O)Nc2ccon2)O[C@@]1(C)C(F)(F)F]

(2R,3S,4S,5R)-3-(3,4-difluoro-2-(2-
hydroxyethoxy)phenyl)-N-(isoxazol-3-yl)-4,5-
dimethyl-5-(trifluoromethyl)tetrahydrofuran-2-
carboxamide

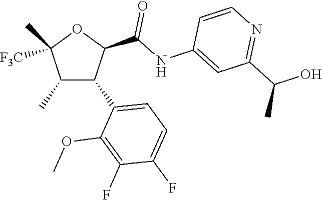 [SMILES: COc1c([C@H]2[C@H](C(=O)Nc3ccnc([C@H](C)O)c3)O[C@@](C)(C(F)(F)F)[C@H]2C)ccc(F)c1F]

(2R,3S,4S,5R)-3-(3,4-difluoro-2-methoxyphenyl)-
N-(2-((S)-1-hydroxyethyl)pyridin-4-yl)-4,5-
dimethyl-5-(trifluoromethyl)tetrahydrofuran-2-
carboxamide

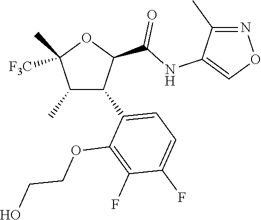 [SMILES: Cc1nocc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-4,5-dimethyl-N-(3-
methylisoxazol-4-yl)-5-(trifluoromethyl)tetrahydrofuran-
2-carboxamide

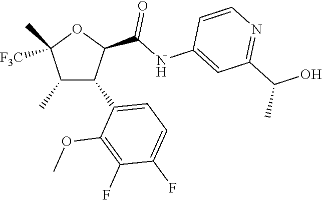 [SMILES: COc1c([C@H]2[C@H](C(=O)Nc3ccnc([C@@H](C)O)c3)O[C@@](C)(C(F)(F)F)[C@H]2C)ccc(F)c1F]

(2R,3S,4S,5R)-3-(3,4-difluoro-2-methoxyphenyl)-
N-(2-((R)-1-hydroxyethyl)pyridin-4-yl)-4,5-
dimethyl-5-(trifluoromethyl)tetrahydrofuran-2-
carboxamide

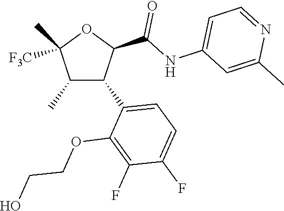 [SMILES: Cc1cc(NC(=O)[C@@H]2O[C@@](C)(C(F)(F)F)[C@@H](C)[C@H]2c2ccc(F)c(F)c2OCCO)ccn1]

(2R,3S,4S,5R)-3-(3,4-difluoro-2-(2-
hydroxyethoxy)phenyl)-4,5-dimethyl-N-(2-
methylpyridin-4-yl)-5-
(trifluoromethyl)tetrahydrofuran-2-carboxamide

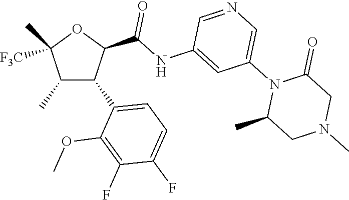 [SMILES: COc1c([C@H]2[C@H](C(=O)Nc3cncc(N4C(=O)CN(C)C[C@H]4C)c3)O[C@@](C)(C(F)(F)F)[C@H]2C)ccc(F)c1F]

(2R,3S,4S,5R)-3-(3,4-difluoro-2-
methoxyphenyl)-N-(5-((R)-2,4-dimethyl-6-
oxopiperazin-1-yl)pyridin-3-yl)-4,5-dimethyl-5-
(trifluoromethyl)tetrahydrofuran-2-carboxamide

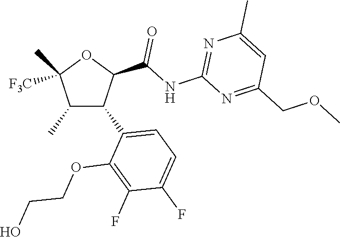 [SMILES: COCc1cc(C)nc(NC(=O)[C@@H]2O[C@@](C)(C(F)(F)F)[C@@H](C)[C@H]2c2ccc(F)c(F)c2OCCO)n1]

(2R,3S,4S,5R)-3-(3,4-difluoro-2-(2-
hydroxyethoxy)phenyl)-N-(4-(methoxymethyl)-6-
methylpyrimidin-2-yl)-4,5-dimethyl-5-
(trifluoromethyl)tetrahydrofuran-2-carboxamide

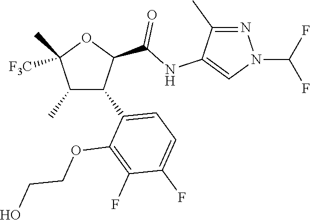 [SMILES: Cc1nn(C(F)F)cc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-N-(1-(difluoromethyl)-3-
methyl-1H-pyrazol-4-yl)-4,5-dimethyl-5-
(trifluoromethyl)tetrahydrofuran-2-carboxamide

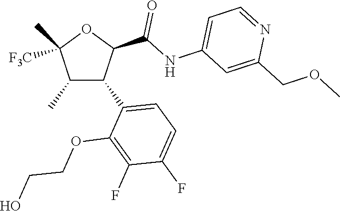 [SMILES: COCc1cc(NC(=O)[C@@H]2O[C@@](C)(C(F)(F)F)[C@@H](C)[C@H]2c2ccc(F)c(F)c2OCCO)ccn1]

(2R,3S,4S,5R)-3-(3,4-difluoro-2-(2-
hydroxyethoxy)phenyl)-N-(2-
(methoxymethyl)pyridin-4-yl)-4,5-dimethyl-5-
(trifluoromethyl)tetrahydrofuran-2-carboxamide

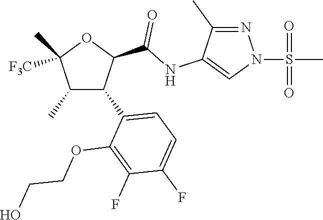 [SMILES: Cc1nn(S(C)(=O)=O)cc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-4,5-dimethyl-N-(3-
methyl-1-(methylsulfonyl)-1H-pyrazol-4-yl)-5-
(trifluoromethyl)tetrahydrofuran-2-carboxamide

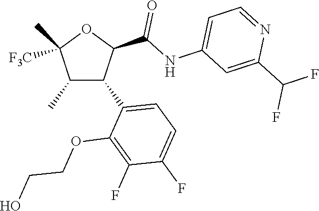 [SMILES: C[C@H]1[C@@H](c2ccc(F)c(F)c2OCCO)[C@H](C(=O)Nc2ccnc(C(F)F)c2)O[C@@]1(C)C(F)(F)F]

(2R,3S,4S,5R)-3-(3,4-difluoro-2-(2-
hydroxyethoxy)phenyl)-N-(2-
(difluoromethyl)pyridin-4-yl)-4,5-dimethyl-5-
(trifluoromethyl)tetrahydrofuran-2-carboxamide

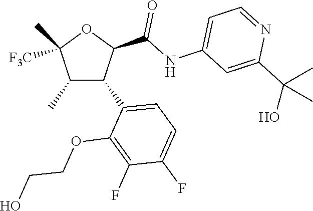 [SMILES: C[C@H]1[C@@H](c2ccc(F)c(F)c2OCCO)[C@H](C(=O)Nc2ccnc(C(C)(C)O)c2)O[C@@]1(C)C(F)(F)F]

(2R,3S,4S,5R)-3-(3,4-difluoro-2-(2-
hydroxyethoxy)phenyl)-N-(2-(2-hydroxypropan-
2-yl)pyridin-4-yl)-4,5-dimethyl-5-
(trifluoromethyl)tetrahydrofuran-2-carboxamide

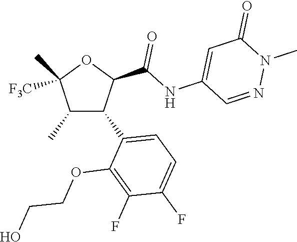 [SMILES: C[C@H]1[C@@H](c2ccc(F)c(F)c2OCCO)[C@H](C(=O)Nc2cnn(C)c(=O)c2)O[C@@]1(C)C(F)(F)F]

(2R,3S,4S,5R)-3-(3,4-difluoro-2-(2-
hydroxyethoxy)phenyl)-4,5-dimethyl-N-(1-
methyl-6-oxo-1,6-dihydropyridazin-4-yl)-5-
(trifluoromethyl)tetrahydrofuran-2-carboxamide

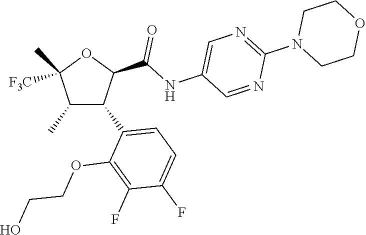 [SMILES: C[C@H]1[C@@H](c2ccc(F)c(F)c2OCCO)[C@H](C(=O)Nc2cnc(N3CCOCC3)nc2)O[C@@]1(C)C(F)(F)F]

(2R,3S,4S,5R)-3-(3,4-difluoro-2-(2-
hydroxyethoxy)phenyl)-4,5-dimethyl-N-(2-
morpholinopyrimidin-5-yl)-5-
(trifluoromethyl)tetrahydrofuran-2-carboxamide

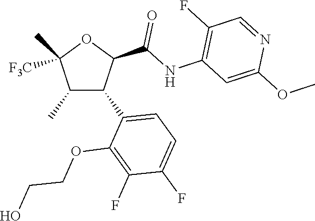 [SMILES: COc1cc(NC(=O)[C@@H]2O[C@@](C)(C(F)(F)F)[C@@H](C)[C@H]2c2ccc(F)c(F)c2OCCO)c(F)cn1]

(2R,3S,4S,5R)-3-(3,4-difluoro-2-(2-
hydroxyethoxy)phenyl)-N-(5-fluoro-2-
methoxypyridin-4-yl)-4,5-dimethyl-5-
(trifluoromethyl)tetrahydrofuran-2-carboxamide

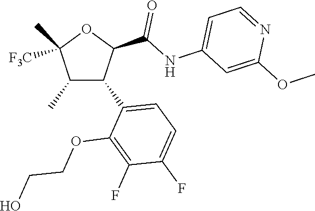 [SMILES: COc1cc(NC(=O)[C@@H]2O[C@@](C)(C(F)(F)F)[C@@H](C)[C@H]2c2ccc(F)c(F)c2OCCO)ccn1]

(2R,3S,4S,5R)-3-(3,4-difluoro-2-(2-
hydroxyethoxy)phenyl)-N-(2-methoxypyridin-4-
yl)-4,5-dimethyl-5-
(trifluoromethyl)tetrahydrofuran-2-carboxamide

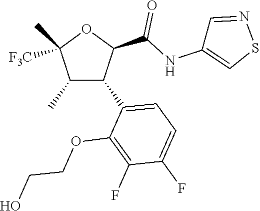 [SMILES: C[C@H]1[C@@H](c2ccc(F)c(F)c2OCCO)[C@H](C(=O)Nc2cnsc2)O[C@@]1(C)C(F)(F)F]

(2R,3S,4S,5R)-3-(3,4-difluoro-2-(2-
hydroxyethoxy)phenyl)-N-(isothiazol-4-yl)-4,5-
dimethyl-5-(trifluoromethyl)tetrahydrofuran-2-
carboxamide

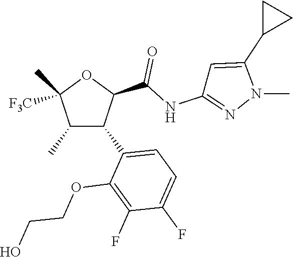 [SMILES: C[C@H]1[C@@H](c2ccc(F)c(F)c2OCCO)[C@H](C(=O)Nc2cc(C3CC3)n(C)n2)O[C@@]1(C)C(F)(F)F]

(2R,3S,4S,5R)-N-(5-cyclopropyl-1-methyl-1H-
pyrazol-3-yl)-3-(3,4-difluoro-2-(2-
hydroxyethoxy)phenyl)-4,5-dimethyl-5-
(trifluoromethyl)tetrahydrofuran-2-carboxamide

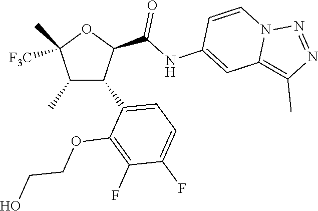 [SMILES: Cc1nnn2ccc(NC(=O)[C@@H]3O[C@@](C)(C(F)(F)F)[C@@H](C)[C@H]3c3ccc(F)c(F)c3OCCO)cc12]

(2R,3S,4S,5R)-3-(3,4-difluoro-2-(2-
hydroxyethoxy)phenyl)-4,5-dimethyl-N-(3-methyl-
[1,2,3]triazolo[1,5-a]pyridin-5-yl)-5-
(trifluoromethyl)tetrahydrofuran-2-carboxamide

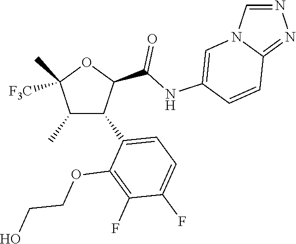 [SMILES: C[C@H]1[C@@H](c2ccc(F)c(F)c2OCCO)[C@H](C(=O)Nc2ccc3nncn3c2)O[C@@]1(C)C(F)(F)F]

(2R,3S,4S,5R)-N-([1,2,4]triazolo[4,3-a]pyridin-6-
yl)-3-(3,4-difluoro-2-(2-hydroxyethoxy)phenyl)-
4,5-dimethyl-5-(trifluoromethyl)tetrahydrofuran-
2-carboxamide

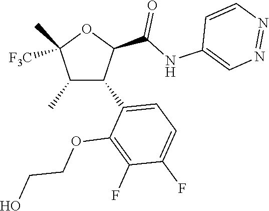 [SMILES: C[C@H]1[C@@H](c2ccc(F)c(F)c2OCCO)[C@H](C(=O)Nc2ccnnc2)O[C@@]1(C)C(F)(F)F]

(2R,3S,4S,5R)-3-(3,4-difluoro-2-(2-
hydroxyethoxy)phenyl)-4,5-dimethyl-N-(pyridazin-
4-yl)-5-(trifluoromethyl)tetrahydrofuran-2-
carboxamide

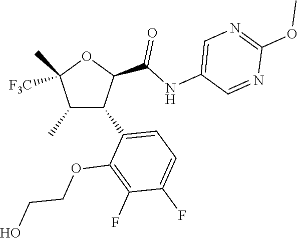 [SMILES: COc1ncc(NC(=O)[C@@H]2O[C@@](C)(C(F)(F)F)[C@@H](C)[C@H]2c2ccc(F)c(F)c2OCCO)cn1]

(2R,3S,4S,5R)-3-(3,4-difluoro-2-(2-
hydroxyethoxy)phenyl)-N-(2-methoxypyrimidin-
5-yl)-4,5-dimethyl-5-
(trifluoromethyl)tetrahydrofuran-2-carboxamide

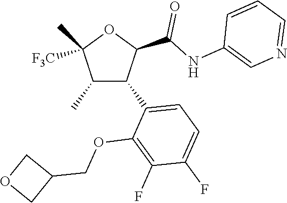 [SMILES: C[C@H]1[C@@H](c2ccc(F)c(F)c2OCC2COC2)[C@H](C(=O)Nc2cccnc2)O[C@@]1(C)C(F)(F)F]

(2R,3S,4S,5R)-3-(3,4-difluoro-2-(oxetan-3-
ylmethoxy)phenyl)-4,5-dimethyl-N-(pyridin-3-
yl)-5-(trifluoromethyl)tetrahydrofuran-2-
carboxamide

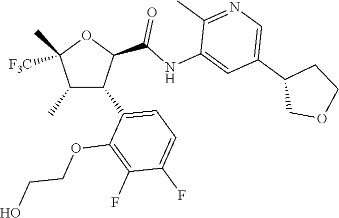 [SMILES: Cc1ncc([C@@H]2CCOC2)cc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-4,5-dimethyl-N-(2-methyl-
5-((R)-tetrahydrofuran-3-yl)pyridin-3-yl)-5-
(trifluoromethyl)tetrahydrofuran-2-carboxamide

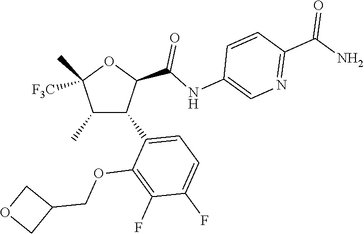 [SMILES: C[C@H]1[C@@H](c2ccc(F)c(F)c2OCC2COC2)[C@H](C(=O)Nc2ccc(C(N)=O)nc2)O[C@@]1(C)C(F)(F)F]

5-((2R,3S,4S,5R)-3-(3,4-difluoro-2-(oxetan-3-
ylmethoxy)phenyl)-4,5-dimethyl-5-
(trifluoromethyl)tetrahydrofuran-2-
carboxamido)picolinamide

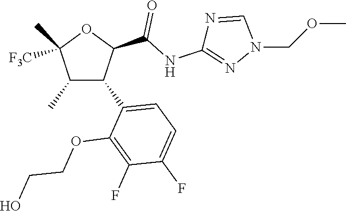 [SMILES: COCn1cnc(NC(=O)[C@@H]2O[C@@](C)(C(F)(F)F)[C@@H](C)[C@H]2c2ccc(F)c(F)c2OCCO)n1]

(2R,3S,4S,5R)-3-(3,4-difluoro-2-(2-
hydroxyethoxy)phenyl)-N-(1-(methoxymethyl)-1H-
1,2,4-triazol-3-yl)-4,5-dimethyl-5-
(trifluoromethyl)tetrahydrofuran-2-carboxamide

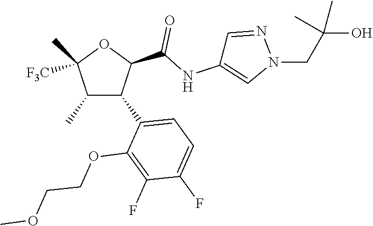 [SMILES: COCCOc1c([C@H]2[C@H](C(=O)Nc3cnn(CC(C)(C)O)c3)O[C@@](C)(C(F)(F)F)[C@H]2C)ccc(F)c1F]

(2R,3S,4S,5R)-3-(3,4-difluoro-2-(2-
methoxyethoxy)phenyl)-N-(1-(2-hydroxy-2-
methylpropyl)-1H-pyrazol-4-yl)-4,5-dimethyl-5-
(trifluoromethyl)tetrahydrofuran-2-carboxamide

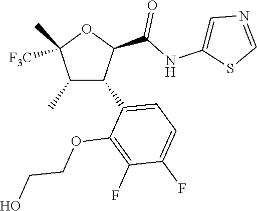 [SMILES: C[C@H]1[C@@H](c2ccc(F)c(F)c2OCCO)[C@H](C(=O)Nc2cncs2)O[C@@]1(C)C(F)(F)F]

(2R,3S,4S,5R)-3-(3,4-difluoro-2-(2-
hydroxyethoxy)phenyl)-4,5-dimethyl-N-(thiazol-
5-yl)-5-(trifluoromethyl)tetrahydrofuran-2-
carboxamide

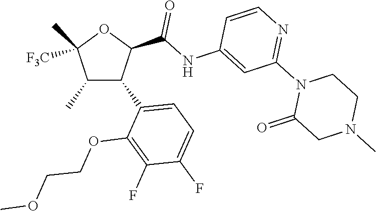

(2R,3S,4S,5R)-3-(3,4-difluoro-2-(2-
methoxyethoxy)phenyl)-4,5-dimethyl-N-(2-(4-
methyl-2-oxopiperazin-1-yl)pyridin-4-yl)-5-
(trifluoromethyl)tetrahydrofuran-2-carboxamide

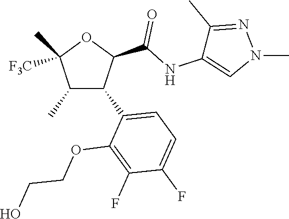 [SMILES: Cc1nn(C)cc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-N-(1,3-dimethyl-1H-pyrazol-
4-yl)-4,5-dimethyl-5-(trifluoromethyl)tetrahydrofuran-
2-carboxamide

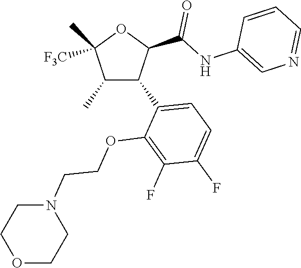 [SMILES: C[C@H]1[C@@H](c2ccc(F)c(F)c2OCCN2CCOCC2)[C@H](C(=O)Nc2cccnc2)O[C@@]1(C)C(F)(F)F]

(2R,3S,4S,5R)-3-(3,4-difluoro-2-(2-
morpholinoethoxy)phenyl)-4,5-dimethyl-N-
(pyridin-3-yl)-5-(trifluoromethyl)tetrahydrofuran-
2-carboxamide

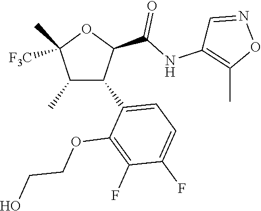 [SMILES: Cc1oncc1NC(=O)[C@@H]1O[C@@](C)(C(F)(F)F)[C@@H](C)[C@H]1c1ccc(F)c(F)c1OCCO]

(2R,3S,4S,5R)-3-(3,4-difluoro-2-(2-
hydroxyethoxy)phenyl)-4,5-dimethyl-N-(5-
methylisoxazol-4-yl)-5-
(trifluoromethyl)tetrahydrofuran-2-carboxamide

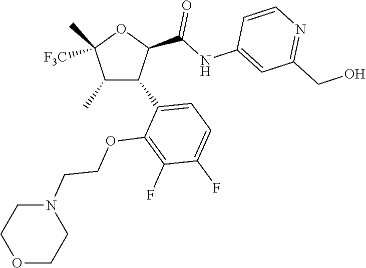 [SMILES: C[C@H]1[C@@H](c2ccc(F)c(F)c2OCCN2CCOCC2)[C@H](C(=O)Nc2ccnc(CO)c2)O[C@@]1(C)C(F)(F)F]

(2R,3S,4S,5R)-3-(3,4-difluoro-2-(2-
morpholinoethoxy)phenyl)-N-(2-
(hydroxymethyl)pyridin-4-yl)-4,5-dimethyl-5-
(trifluoromethyl)tetrahydrofuran-2-carboxamide

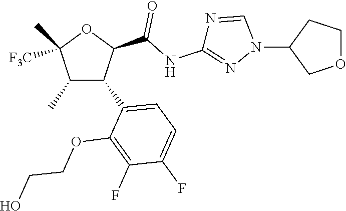 [SMILES: C[C@H]1[C@@H](c2ccc(F)c(F)c2OCCO)[C@H](C(=O)Nc2ncn(C3CCOC3)n2)O[C@@]1(C)C(F)(F)F]

(2R,3S,4S,5R)-3-(3,4-difluoro-2-(2-
hydroxyethoxy)phenyl)-4,5-dimethyl-N-(1-
(tetrahydrofuran-3-yl)-1H-1,2,4-triazol-3-yl)-5-
(trifluoromethyl)tetrahydrofuran-2-carboxamide

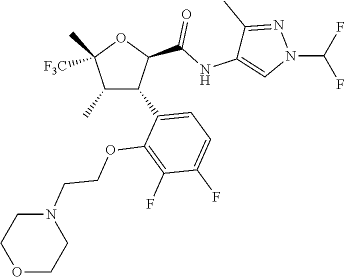 [SMILES: Cc1nn(C(F)F)cc1NC(=O)[C@@H]1O[C@@](C)(C(F)(F)F)[C@@H](C)[C@H]1c1ccc(F)c(F)c1OCCN1CCOCC1]

(2R,3S,4S,5R)-3-(3,4-difluoro-2-(2-
morpholinoethoxy)phenyl)-N-(1-(difluoromethyl)-
3-methyl-1H-pyrazol-4-yl)-4,5-dimethyl-5-
(trifluoromethyl)tetrahydrofuran-2-carboxamide

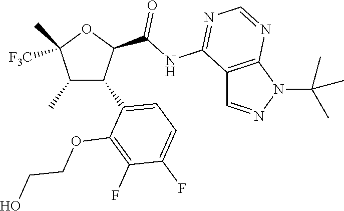 [SMILES: C[C@H]1[C@@H](c2ccc(F)c(F)c2OCCO)[C@H](C(=O)Nc2ncnc3c2cnn3C(C)(C)C)O[C@@]1(C)C(F)(F)F]

(2R,3S,4S,5R)-N-(1-(tert-butyl)-1H-
pyrazolo[3,4-d]pyrimidin-4-yl)-3-(3,4-difluoro-2-
(2-hydroxyethoxy)phenyl)-4,5-dimethyl-5-
(trifluoromethyl)tetrahydrofuran-2-carboxamide

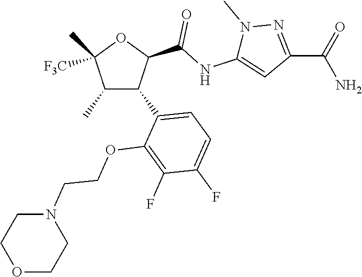 [SMILES: C[C@H]1[C@@H](c2ccc(F)c(F)c2OCCN2CCOCC2)[C@H](C(=O)Nc2cc(C(N)=O)nn2C)O[C@@]1(C)C(F)(F)F]

5-((2R,3S,4S,5R)-3-(3,4-difluoro-2-(2-
morpholinoethoxy)phenyl)-4,5-dimethyl-5-
(trifluoromethyl)tetrahydrofuran-2-
carboxamido)-1-methyl-1H-pyrazole-3-
carboxamide

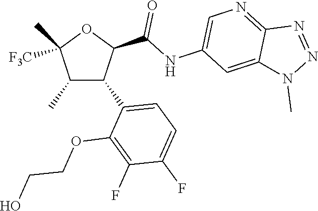 [SMILES: C[C@H]1[C@@H](c2ccc(F)c(F)c2OCCO)[C@H](C(=O)Nc2cnc3nnn(C)c3c2)O[C@@]1(C)C(F)(F)F]

(2R,3S,4S,5R)-3-(3,4-difluoro-2-(2-
hydroxyethoxy)phenyl)-4,5-dimethyl-N-(1-methyl-
1H-[1,2,3]triazolo[4,5-b]pyridin-6-yl)-5-
(trifluoromethyl)tetrahydrofuran-2-carboxamide

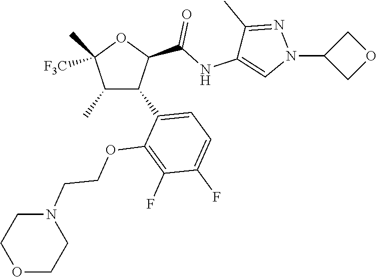 [SMILES: Cc1nn(C2COC2)cc1NC(=O)[C@@H]1O[C@@](C)(C(F)(F)F)[C@@H](C)[C@H]1c1ccc(F)c(F)c1OCCN1CCOCC1]

(2R,3S,4S,5R)-3-(3,4-difluoro-2-(2-
morpholinoethoxy)phenyl)-4,5-dimethyl-N-(3-
methyl-1-(oxetan-3-yl)-1H-pyrazol-4-yl)-5-
(trifluoromethyl)tetrahydrofuran-2-carboxamide

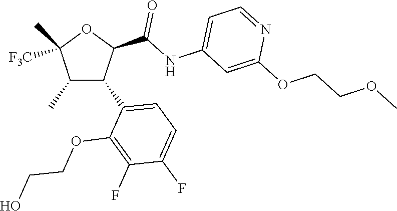 [SMILES: COCCOc1cc(NC(=O)[C@@H]2O[C@@](C)(C(F)(F)F)[C@@H](C)[C@H]2c2ccc(F)c(F)c2OCCO)ccn1]

(2R,3S,4S,5R)-3-(3,4-difluoro-2-(2-
hydroxyethoxy)phenyl)-N-(2-(2-
methoxyethoxy)pyridin-4-yl)-4,5-dimethyl-5-
(trifluoromethyl)tetrahydrofuran-2-carboxamide

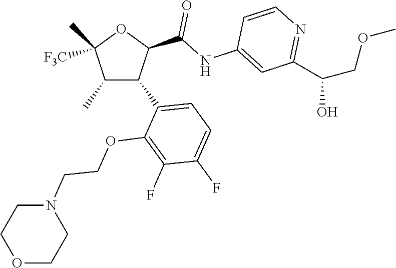 [SMILES: COC[C@H](O)c1cc(NC(=O)[C@@H]2O[C@@](C)(C(F)(F)F)[C@@H](C)[C@H]2c2ccc(F)c(F)c2OCCN2CCOCC2)ccn1]

(2R,3S,4S,5R)-3-(3,4-difluoro-2-(2-
morpholinoethoxy)phenyl)-N-(2-((S)-1-hydroxy-2-
methoxyethyl)pyridin-4-yl)-4,5-dimethyl-5-
(trifluoromethyl)tetrahydrofuran-2-carboxamide

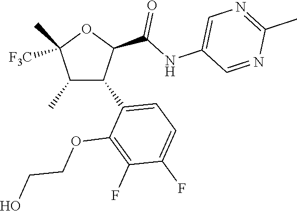 [SMILES: Cc1ncc(NC(=O)[C@@H]2O[C@@](C)(C(F)(F)F)[C@@H](C)[C@H]2c2ccc(F)c(F)c2OCCO)cn1]

(2R,3S,4S,5R)-3-(3,4-difluoro-2-(2-
hydroxyethoxy)phenyl)-4,5-dimethyl-N-(2-
methylpyrimidin-5-yl)-5-
(trifluoromethyl)tetrahydrofuran-2-carboxamide

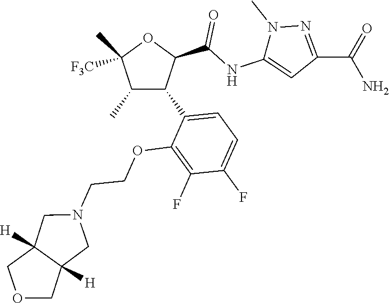 [SMILES: C[C@H]1[C@@H](c2ccc(F)c(F)c2OCCN2C[C@H]3COC[C@H]3C2)[C@H](C(=O)Nc2cc(C(N)=O)nn2C)O[C@@]1(C)C(F)(F)F]

5-((2R,3S,4S,5R)-3-(3,4-difluoro-2-(2-
((3aR,6aS)-tetrahydro-1H-furo[3,4-c]pyrrol-
5(3H)-yl)ethoxy)phenyl)-4,5-dimethyl-5-
(trifluoromethyl)tetrahydrofuran-2-
carboxamido)-1-methyl-1H-pyrazole-3-
carboxamide

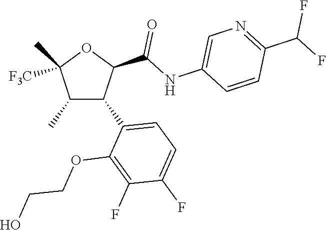 [SMILES: C[C@H]1[C@@H](c2ccc(F)c(F)c2OCCO)[C@H](C(=O)Nc2ccc(C(F)F)nc2)O[C@@]1(C)C(F)(F)F]

(2R,3S,4S,5R)-3-(3,4-difluoro-2-(2-
hydroxyethoxy)phenyl)-N-(6-
(difluoromethyl)pyridin-3-yl)-4,5-dimethyl-5-
(trifluoromethyl)tetrahydrofuran-2-carboxamide

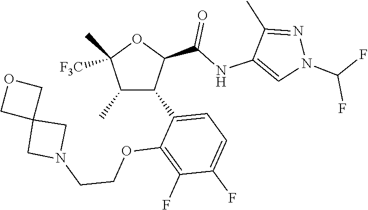 [SMILES: Cc1nn(C(F)F)cc1NC(=O)[C@@H]1O[C@@](C)(C(F)(F)F)[C@@H](C)[C@H]1c1ccc(F)c(F)c1OCCN1CC2(COC2)C1]

(2R,3S,4S,5R)-3-(2-(2-(2-oxa-6-
azaspiro[3.3]heptan-6-yl)ethoxy)-3,4-
difluorophenyl)-N-(1-(difluoromethyl)-3-methyl-
1H-pyrazol-4-yl)-4,5-dimethyl-5-
(trifluoromethyl)tetrahydrofuran-2-carboxamide

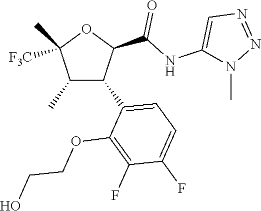 [SMILES: C[C@H]1[C@@H](c2ccc(F)c(F)c2OCCO)[C@H](C(=O)Nc2cnnn2C)O[C@@]1(C)C(F)(F)F]

(2R,3S,4S,5R)-3-(3,4-difluoro-2-(2-
hydroxyethoxy)phenyl)-4,5-dimethyl-N-(1-
methyl-1H-1,2,3-triazol-5-yl)-5-
(trifluoromethyl)tetrahydrofuran-2-carboxamide

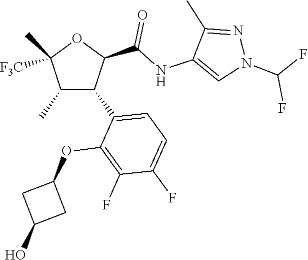 [SMILES: Cc1nn(C(F)F)cc1NC(=O)[C@@H]1O[C@@](C)(C(F)(F)F)[C@@H](C)[C@H]1c1ccc(F)c(F)c1O[C@H]1C[C@@H](O)C1]

(2R,3S,4S,5R)-3-(3,4-difluoro-2-((1s,3R)-3-
hydroxycyclobutoxy)phenyl)-N-(1-
(difluoromethyl)-3-methyl-1H-pyrazol-4-yl)-4,5-
dimethyl-5-(trifluoromethyl)tetrahydrofuran-2-
carboxamide

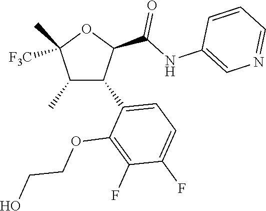 [SMILES: C[C@H]1[C@@H](c2ccc(F)c(F)c2OCCO)[C@H](C(=O)Nc2cccnc2)O[C@@]1(C)C(F)(F)F]

(2R,3S,4S,5R)-3-(3,4-difluoro-2-(2-
hydroxyethoxy)phenyl)-4,5-dimethyl-N-(pyridin-3-
yl)-5-(trifluoromethyl)tetrahydrofuran-2-
carboxamide

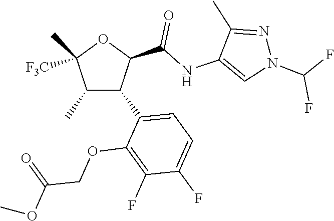 [SMILES: COC(=O)COc1c([C@H]2[C@H](C(=O)Nc3cn(C(F)F)nc3C)O[C@@](C)(C(F)(F)F)[C@H]2C)ccc(F)c1F]

methyl 2-(6-((2R,3S,4S,5R)-2-((1-(difluoromethyl)-
3-methyl-1H-pyrazol-4-yl)carbamoyl)-4,5-dimethyl-
5-(trifluoromethyl)tetrahydrofuran-3-yl)-2,3-
difluorophenoxy)acetate

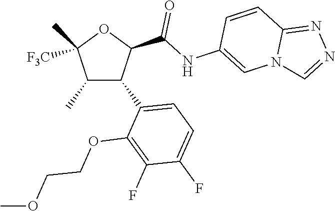 [SMILES: COCCOc1c([C@H]2[C@H](C(=O)Nc3ccc4nncn4c3)O[C@@](C)(C(F)(F)F)[C@H]2C)ccc(F)c1F]

(2R,3S,4S,5R)-N-([1,2,4]triazolo[4,3-a]pyridin-6-
yl)-3-(3,4-difluoro-2-(2-methoxyethoxy)phenyl)-
4,5-dimethyl-5-(trifluoromethyl)tetrahydrofuran-
2-carboxamide

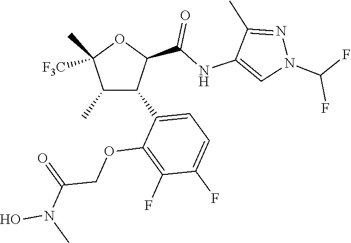 [SMILES: Cc1nn(C(F)F)cc1NC(=O)[C@@H]1O[C@@](C)(C(F)(F)F)[C@@H](C)[C@H]1c1ccc(F)c(F)c1OCC(=O)N(C)O]

(2R,3S,4S,5R)-3-(3,4-difluoro-2-(2-
(hydroxy(methyl)amino)-2-oxoethoxy)phenyl)-
N-(1-(difluoromethyl)-3-methyl-1H-pyrazol-4-
yl)-4,5-dimethyl-5-
(trifluoromethyl)tetrahydrofuran-2-carboxamide

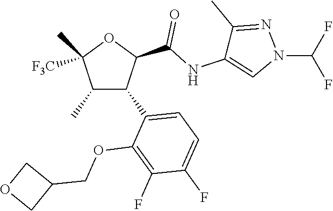 [SMILES: Cc1nn(C(F)F)cc1NC(=O)[C@@H]1O[C@@](C)(C(F)(F)F)[C@@H](C)[C@H]1c1ccc(F)c(F)c1OCC1COC1]

(2R,3S,4S,5R)-3-(3,4-difluoro-2-(oxetan-3-
ylmethoxy)phenyl)-N-(1-(difluoromethyl)-3-
methyl-1H-pyrazol-4-yl)-4,5-dimethyl-5-
(trifluoromethyl)tetrahydrofuran-2-carboxamide

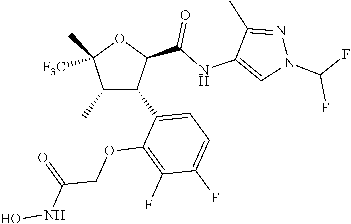 [SMILES: Cc1nn(C(F)F)cc1NC(=O)[C@@H]1O[C@@](C)(C(F)(F)F)[C@@H](C)[C@H]1c1ccc(F)c(F)c1OCC(=O)NO]

(2R,3S,4S,5R)-3-(3,4-difluoro-2-(2-
(hydroxy(methyl)amino)-2-oxoethoxy)phenyl)-N-
(1-(difluoromethyl)-3-methyl-1H-pyrazol-4-yl)-
4,5-dimethyl-5-(trifluoromethyl)tetrahydrofuran-2-
carboxamide

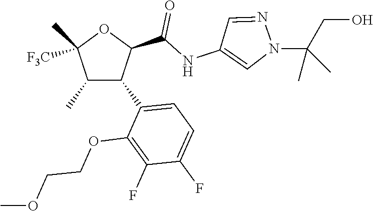 [SMILES: COCCOc1c([C@H]2[C@H](C(=O)Nc3cnn(C(C)(C)CO)c3)O[C@@](C)(C(F)(F)F)[C@H]2C)ccc(F)c1F]

(2R,3S,4S,5R)-3-(3,4-difluoro-2-(2-
methoxyethoxy)phenyl)-N-(1-(1-hydroxy-2-
methylpropan-2-yl)-1H-pyrazol-4-yl)-4,5-
dimethyl-5-(trifluoromethyl)tetrahydrofuran-2-
carboxamide

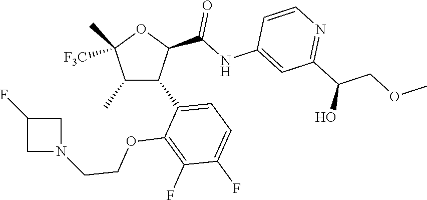 [SMILES: COC[C@@H](O)c1cc(NC(=O)[C@@H]2O[C@@](C)(C(F)(F)F)[C@@H](C)[C@H]2c2ccc(F)c(F)c2OCCN2CC(F)C2)ccn1]

(2R,3S,4S,5R)-3-(3,4-difluoro-2-(2-(3-
fluoroazetidin-1-yl)ethoxy)phenyl)-N-(2-((S)-1-
hydroxy-2-methoxyethyl)pyridin-4-yl)-4,5-dimethyl-
5-(trifluoromethyl)tetrahydrofuran-2-carboxamide

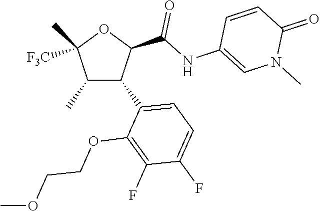 [SMILES: COCCOc1c([C@H]2[C@H](C(=O)Nc3ccc(=O)n(C)c3)O[C@@](C)(C(F)(F)F)[C@H]2C)ccc(F)c1F]

(2R,3S,4S,5R)-3-(3,4-difluoro-2-(2-
methoxyethoxy)phenyl)-4,5-dimethyl-N-(1-
methyl-6-oxo-1,6-dihydropyridin-3-yl)-5-
(trifluoromethyl)tetrahydrofuran-2-carboxamide

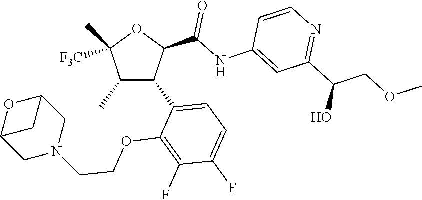 [SMILES: COC[C@@H](O)c1cc(NC(=O)[C@@H]2O[C@@](C)(C(F)(F)F)[C@@H](C)[C@H]2c2ccc(F)c(F)c2OCCN2CC3CC(C2)O3)ccn1]

(2R,3S,4S,5R)-3-(2-(2-(6-oxa-3-
azabicyclo[3.1.1]heptan-3-yl)ethoxy)-3,4-
difluorophenyl)-N-(2-((S)-1-hydroxy-2-
methoxyethyl)pyridin-4-yl)-4,5-dimethyl-5-
(trifluoromethyl)tetrahydrofuran-2-carboxamide

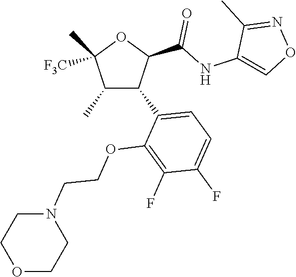 [SMILES: Cc1nocc1NC(=O)[C@@H]1O[C@@](C)(C(F)(F)F)[C@@H](C)[C@H]1c1ccc(F)c(F)c1OCCN1CCOCC1]

(2R,3S,4S,5R)-3-(3,4-difluoro-2-(2-
morpholinoethoxy)phenyl)-4,5-dimethyl-N-(3-
methylisoxazol-4-yl)-5-
(trifluoromethyl)tetrahydrofuran-2-carboxamide

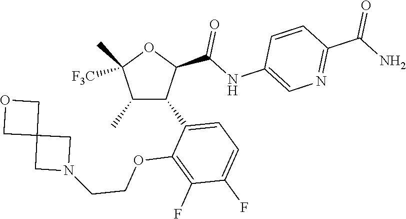 [SMILES: C[C@H]1[C@@H](c2ccc(F)c(F)c2OCCN2CC3(COC3)C2)[C@H](C(=O)Nc2ccc(C(N)=O)nc2)O[C@@]1(C)C(F)(F)F]

5-((2R,3S,4S,5R)-3-(2-(2-(2-oxa-6-
azaspiro[3.3]heptan-6-yl)ethoxy)-3,4-difluorophenyl)-
4,5-dimethyl-5-(trifluoromethyl)tetrahydrofuran-2-
carboxamido)picolinamide

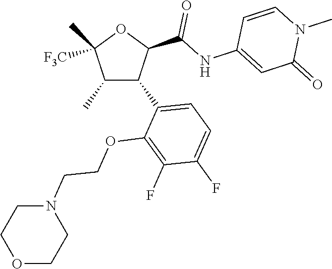 [SMILES: C[C@H]1[C@@H](c2ccc(F)c(F)c2OCCN2CCOCC2)[C@H](C(=O)Nc2ccn(C)c(=O)c2)O[C@@]1(C)C(F)(F)F]

(2R,3S,4S,5R)-3-(3,4-difluoro-2-(2-
morpholinoethoxy)phenyl)-4,5-dimethyl-N-(1-
methyl-2-oxo-1,2-dihydropyridin-4-yl)-5-
(trifluoromethyl)tetrahydrofuran-2-carboxamide

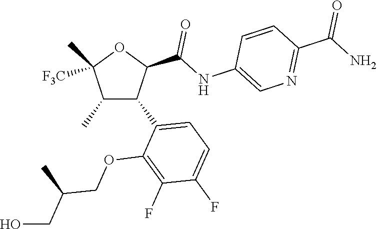 [SMILES: C[C@H](CO)COc1c([C@H]2[C@H](C(=O)Nc3ccc(C(N)=O)nc3)O[C@@](C)(C(F)(F)F)[C@H]2C)ccc(F)c1F]

5-((2R,3S,4S,5R)-3-(3,4-difluoro-2-((R)-3-
hydroxy-2-methylpropoxy)phenyl)-4,5-dimethyl-
5-(trifluoromethyl)tetrahydrofuran-2-
carboxamido)picolinamide

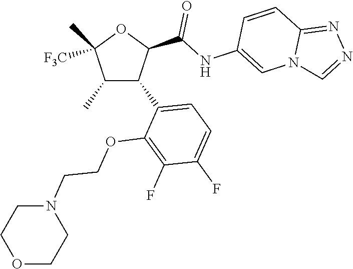 [SMILES: C[C@H]1[C@@H](c2ccc(F)c(F)c2OCCN2CCOCC2)[C@H](C(=O)Nc2ccc3nncn3c2)O[C@@]1(C)C(F)(F)F]

(2R,3S,4S,5R)-N-([1,2,4]triazolo[4,3-a]pyridin-6-
yl)-3-(3,4-difluoro-2-(2-
morpholinoethoxy)phenyl)-4,5-dimethyl-5-
(trifluoromethyl)tetrahydrofuran-2-carboxamide

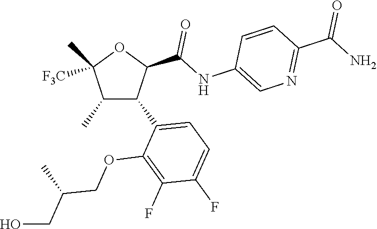 [SMILES: C[C@@H](CO)COc1c([C@H]2[C@H](C(=O)Nc3ccc(C(N)=O)nc3)O[C@@](C)(C(F)(F)F)[C@H]2C)ccc(F)c1F]

5-((2R,3S,4S,5R)-3-(3,4-difluoro-2-((S)-3-
hydroxy-2-methylpropoxy)phenyl)-4,5-dimethyl-
5-(trifluoromethyl)tetrahydrofuran-2-
carboxamido)picolinamide

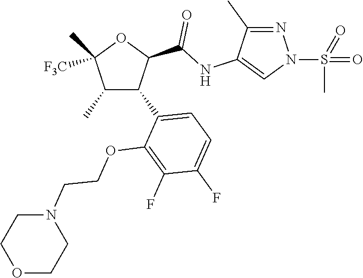 [SMILES: Cc1nn(S(C)(=O)=O)cc1NC(=O)[C@@H]1O[C@@](C)(C(F)(F)F)[C@@H](C)[C@H]1c1ccc(F)c(F)c1OCCN1CCOCC1]

(2R,3S,4S,5R)-3-(3,4-difluoro-2-(2-
morpholinoethoxy)phenyl)-4,5-dimethyl-N-(3-
methyl-1-(methylsulfonyl)-1H-pyrazol-4-yl)-5-
(trifluoromethyl)tetrahydrofuran-2-carboxamide

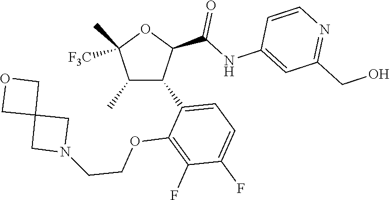 [SMILES: C[C@H]1[C@@H](c2ccc(F)c(F)c2OCCN2CC3(COC3)C2)[C@H](C(=O)Nc2ccnc(CO)c2)O[C@@]1(C)C(F)(F)F]

(2R,3S,4S,5R)-3-(2-(2-(2-oxa-6-
azaspiro[3.3]heptan-6-yl)ethoxy)-3,4-
difluorophenyl)-N-(2-(hydroxymethyl)pyridin-4-yl)-
4,5-dimethyl-5-(trifluoromethyl)tetrahydrofuran-2-
carboxamide

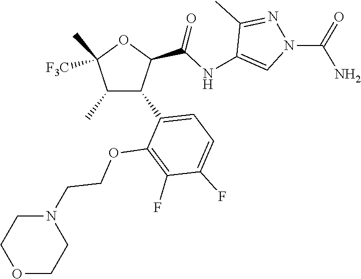 [SMILES: Cc1nn(C(N)=O)cc1NC(=O)[C@@H]1O[C@@](C)(C(F)(F)F)[C@@H](C)[C@H]1c1ccc(F)c(F)c1OCCN1CCOCC1]

4-((2R,3S,4S,5R)-3-(3,4-difluoro-2-(2-
morpholinoethoxy)phenyl)-4,5-dimethyl-5-
(trifluoromethyl)tetrahydrofuran-2-carboxamido)-
3-methyl-1H-pyrazole-1-carboxamide

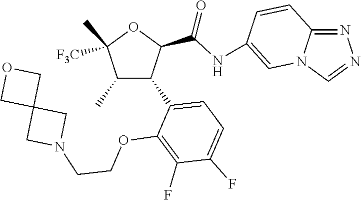 [SMILES: C[C@H]1[C@@H](c2ccc(F)c(F)c2OCCN2CC3(COC3)C2)[C@H](C(=O)Nc2ccc3nncn3c2)O[C@@]1(C)C(F)(F)F]

(2R,3S,4S,5R)-3-(2-(2-(2-oxa-6-
azaspiro[3.3]heptan-6-yl)ethoxy)-3,4-
difluorophenyl)-N-([1,2,4]triazolo[4,3-a]pyridin-
6-yl)-4,5-dimethyl-5-
(trifluoromethyl)tetrahydrofuran-2-carboxamide

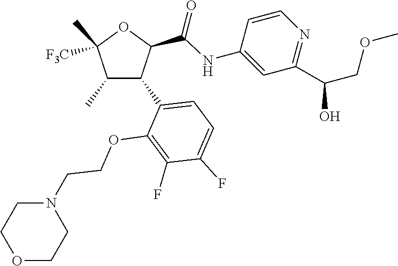 [SMILES: COC[C@@H](O)c1cc(NC(=O)[C@@H]2O[C@@](C)(C(F)(F)F)[C@@H](C)[C@H]2c2ccc(F)c(F)c2OCCN2CCOCC2)ccn1]

(2R,3S,4S,5R)-3-(3,4-difluoro-2-(2-
morpholinoethoxy)phenyl)-N-(2-((S)-1-hydroxy-2-
methoxyethyl)pyridin-4-yl)-4,5-dimethyl-5-
(trifluoromethyl)tetrahydrofuran-2-carboxamide

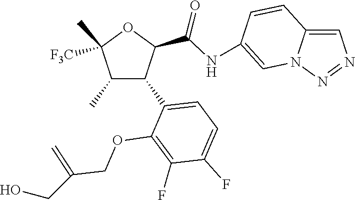 [SMILES: C=C(CO)COc1c([C@H]2[C@H](C(=O)Nc3ccc4cnnn4c3)O[C@@](C)(C(F)(F)F)[C@H]2C)ccc(F)c1F]

(2R,3S,4S,5R)-N-([1,2,3]triazolo[1,5-a]pyridin-
6-yl)-3-(3,4-difluoro-2-((2-
(hydroxymethyl)allyl)oxy)phenyl)-4,5-dimethyl-
5-(trifluoromethyl)tetrahydrofuran-2-
carboxamide

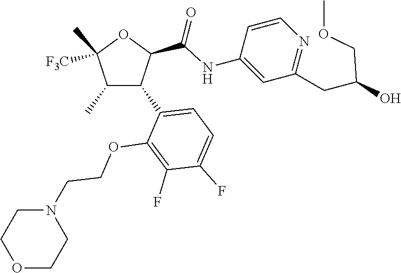 [SMILES: COC[C@@H](O)Cc1cc(NC(=O)[C@@H]2O[C@@](C)(C(F)(F)F)[C@@H](C)[C@H]2c2ccc(F)c(F)c2OCCN2CCOCC2)ccn1]

(2R,3S,4S,5R)-3-(3,4-difluoro-2-(2-
morpholinoethoxy)phenyl)-N-(2-((S)-2-hydroxy-3-
methoxypropyl)pyridin-4-yl)-4,5-dimethyl-5-
(trifluoromethyl)tetrahydrofuran-2-carboxamide

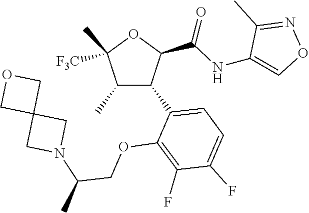 [SMILES: Cc1nocc1NC(=O)[C@@H]1O[C@@](C)(C(F)(F)F)[C@@H](C)[C@H]1c1ccc(F)c(F)c1OC[C@@H](C)N1CC2(COC2)C1]

(2R,3S,4S,5R)-3-(2-((R)-2-(2-oxa-6-
azaspiro[3.3]heptan-6-yl)propoxy)-3,4-
difluorophenyl)-4,5-dimethyl-N-(3-methylisoxazol-
4-yl)-5-(trifluoromethyl)tetrahydrofuran-2-
carboxamide

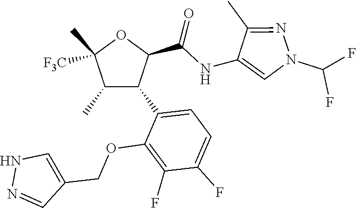 [SMILES: Cc1nn(C(F)F)cc1NC(=O)[C@@H]1O[C@@](C)(C(F)(F)F)[C@@H](C)[C@H]1c1ccc(F)c(F)c1OCc1cn[nH]c1]

(2R,3S,4S,5R)-3-(2-((1H-pyrazol-4-
yl)methoxy)-3,4-difluorophenyl)-N-(1-
(difluoromethyl)-3-methyl-1H-pyrazol-4-yl)-4,5-
dimethyl-5-(trifluoromethyl)tetrahydrofuran-2-
carboxamide

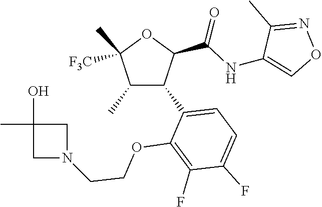 [SMILES: Cc1nocc1NC(=O)[C@@H]1O[C@@](C)(C(F)(F)F)[C@@H](C)[C@H]1c1ccc(F)c(F)c1OCCN1CC(C)(O)C1]

(2R,3S,4S,5R)-3-(3,4-difluoro-2-(2-(3-hydroxy-
3-methylazetidin-1-yl)ethoxy)phenyl)-4,5-
dimethyl-N-(3-methylisoxazol-4-yl)-5-
(trifluoromethyl)tetrahydrofuran-2-carboxamide

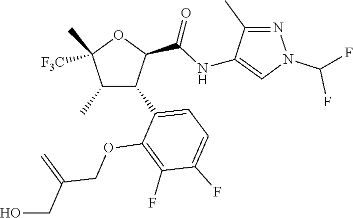 [SMILES: C=C(CO)COc1c([C@H]2[C@H](C(=O)Nc3cn(C(F)F)nc3C)O[C@@](C)(C(F)(F)F)[C@H]2C)ccc(F)c1F]

(2R,3S,4S,5R)-3-(3,4-difluoro-2-((2-
(hydroxymethyl)allyl)oxy)phenyl)-N-(1-
(difluoromethyl)-3-methyl-1H-pyrazol-4-yl)-4,5-
dimethyl-5-(trifluoromethyl)tetrahydrofuran-2-
carboxamide

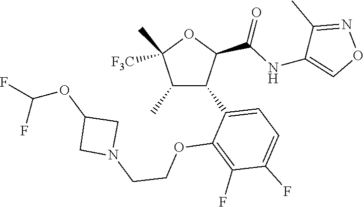 [SMILES: Cc1nocc1NC(=O)[C@@H]1O[C@@](C)(C(F)(F)F)[C@@H](C)[C@H]1c1ccc(F)c(F)c1OCCN1CC(OC(F)F)C1]

(2R,3S,4S,5R)-3-(2-(2-(3-
(difluoromethoxy)azetidin-1-yl)ethoxy)-3,4-
difluorophenyl)-4,5-dimethyl-N-(3-methylisoxazol-
4-yl)-5-(trifluoromethyl)tetrahydrofuran-2-
carboxamide

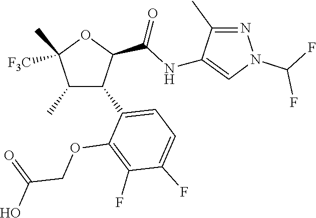 [SMILES: Cc1nn(C(F)F)cc1NC(=O)[C@@H]1O[C@@](C)(C(F)(F)F)[C@@H](C)[C@H]1c1ccc(F)c(F)c1OCC(=O)O]

2-(6-((2R,3S,4S,5R)-2-((1-(difluoromethyl)-3-
methyl-1H-pyrazol-4-yl)carbamoyl)-4,5-
dimethyl-5-(trifluoromethyl)tetrahydrofuran-3-
yl)-2,3-difluorophenoxy)acetic acid

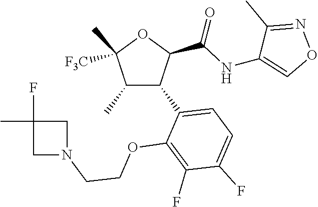 [SMILES: Cc1nocc1NC(=O)[C@@H]1O[C@@](C)(C(F)(F)F)[C@@H](C)[C@H]1c1ccc(F)c(F)c1OCCN1CC(C)(F)C1]

(2R,3S,4S,5R)-3-(3,4-difluoro-2-(2-(3-fluoro-3-
methylazetidin-1-yl)ethoxy)phenyl)-4,5-
dimethyl-N-(3-methylisoxazol-4-yl)-5-
(trifluoromethyl)tetrahydrofuran-2-carboxamide

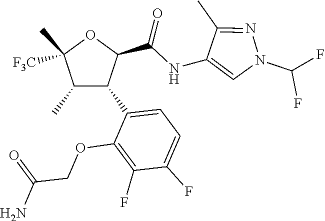 [SMILES: Cc1nn(C(F)F)cc1NC(=O)[C@@H]1O[C@@](C)(C(F)(F)F)[C@@H](C)[C@H]1c1ccc(F)c(F)c1OCC(N)=O]

(2R,3S,4S,5R)-3-(2-(2-amino-2-oxoethoxy)-3,4-
difluorophenyl)-N-(1-(difluoromethyl)-3-methyl-
1H-pyrazol-4-yl)-4,5-dimethyl-5-
(trifluoromethyl)tetrahydrofuran-2-carboxamide

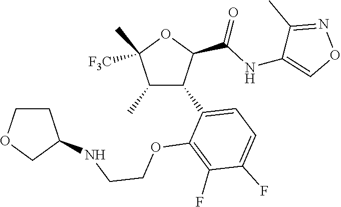 [SMILES: Cc1nocc1NC(=O)[C@@H]1O[C@@](C)(C(F)(F)F)[C@@H](C)[C@H]1c1ccc(F)c(F)c1OCCN[C@@H]1CCOC1]

(2R,3S,4S,5R)-3-(3,4-difluoro-2-(2-(((R)-
tetrahydrofuran-3-yl)amino)ethoxy)phenyl)-4,5-
dimethyl-N-(3-methylisoxazol-4-yl)-5-
(trifluoromethyl)tetrahydrofuran-2-carboxamide

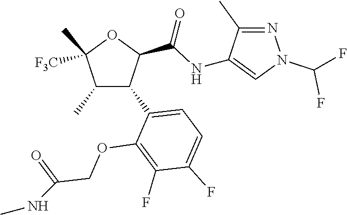 [SMILES: CNC(=O)COc1c([C@H]2[C@H](C(=O)Nc3cn(C(F)F)nc3C)O[C@@](C)(C(F)(F)F)[C@H]2C)ccc(F)c1F]

(2R,3S,4S,5R)-3-(3,4-difluoro-2-(2-(methylamino)-2-
oxoethoxy)phenyl)-N-(1-(difluoromethyl)-3-methyl-
1H-pyrazol-4-yl)-4,5-dimethyl-5-
(trifluoromethyl)tetrahydrofuran-2-carboxamide

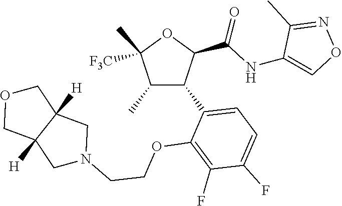 [SMILES: Cc1nocc1NC(=O)[C@@H]1O[C@@](C)(C(F)(F)F)[C@@H](C)[C@H]1c1ccc(F)c(F)c1OCCN1C[C@H]2COC[C@H]2C1]

(2R,3S,4S,5R)-3-(3,4-difluoro-2-(2-((3aR,6aS)-
tetrahydro-1H-furo[3,4-c]pyrrol-5(3H)-
yl)ethoxy)phenyl)-4,5-dimethyl-N-(3-
methylisoxazol-4-yl)-5-
(trifluoromethyl)tetrahydrofuran-2-carboxamide

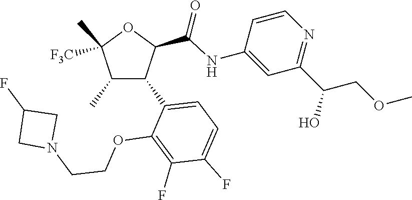 [SMILES: COC[C@H](O)c1cc(NC(=O)[C@@H]2O[C@@](C)(C(F)(F)F)[C@@H](C)[C@H]2c2ccc(F)c(F)c2OCCN2CC(F)C2)ccn1]

(2R,3S,4S,5R)-3-(3,4-difluoro-2-(2-(3-fluoroazetidin-
1-yl)ethoxy)phenyl)-N-(2-((R)-1-hydroxy-2-
methoxyethyl)pyridin-4-yl)-4,5-dimethyl-5-
(trifluoromethyl)tetrahydrofuran-2-carboxamide

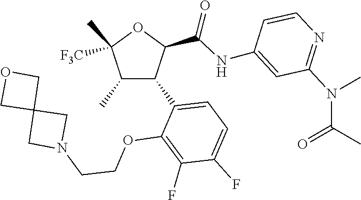 [SMILES: CC(=O)N(C)c1cc(NC(=O)[C@@H]2O[C@@](C)(C(F)(F)F)[C@@H](C)[C@H]2c2ccc(F)c(F)c2OCCN2CC3(COC3)C2)ccn1]

(2R,3S,4S,5R)-3-(2-(2-(2-oxa-6-
azaspiro[3.3]heptan-6-yl)ethoxy)-3,4-
difluorophenyl)-4,5-dimethyl-N-(2-(N-
methylacetamido)pyridin-4-yl)-5-
(trifluoromethyl)tetrahydrofuran-2-carboxamide

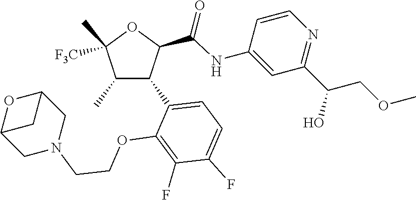 [SMILES: COC[C@H](O)c1cc(NC(=O)[C@@H]2O[C@@](C)(C(F)(F)F)[C@@H](C)[C@H]2c2ccc(F)c(F)c2OCCN2CC3CC(C2)O3)ccn1]

(2R,3S,4S,5R)-3-(2-(2-(6-oxa-3-
azabicyclo[3.1.1]heptan-3-yl)ethoxy)-3,4-
difluorophenyl)-N-(2-((R)-1-hydroxy-2-
methoxyethyl)pyridin-4-yl)-4,5-dimethyl-5-
(trifluoromethyl)tetrahydrofuran-2-carboxamide

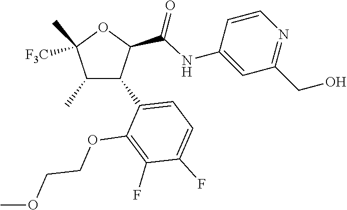 [SMILES: COCCOc1c([C@H]2[C@H](C(=O)Nc3ccnc(CO)c3)O[C@@](C)(C(F)(F)F)[C@H]2C)ccc(F)c1F]

(2R,3S,4S,5R)-3-(3,4-difluoro-2-(2-
methoxyethoxy)phenyl)-N-(2-
(hydroxymethyl)pyridin-4-yl)-4,5-dimethyl-5-
(trifluoromethyl)tetrahydrofuran-2-carboxamide

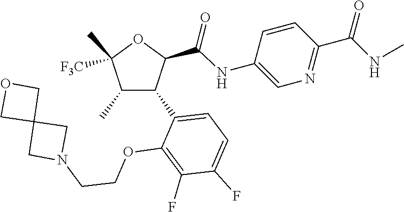 [SMILES: CNC(=O)c1ccc(NC(=O)[C@@H]2O[C@@](C)(C(F)(F)F)[C@@H](C)[C@H]2c2ccc(F)c(F)c2OCCN2CC3(COC3)C2)cn1]

5-((2R,3S,4S,5R)-3-(2-(2-(2-oxa-6-
azaspiro[3.3]heptan-6-yl)ethoxy)-3,4-
difluorophenyl)-4,5-dimethyl-5-
(trifluoromethyl)tetrahydrofuran-2-
carboxamido)-N-methylpicolinamide

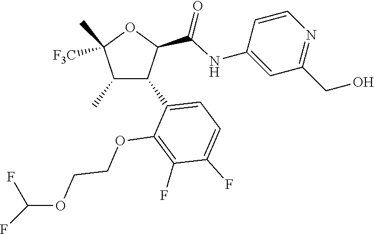 [SMILES: C[C@H]1[C@@H](c2ccc(F)c(F)c2OCCOC(F)F)[C@H](C(=O)Nc2ccnc(CO)c2)O[C@@]1(C)C(F)(F)F]

(2R,3S,4S,5R)-3-(2-(2-(difluoromethoxy)ethoxy)-
3,4-difluorophenyl)-N-(2-(hydroxymethyl)pyridin-
4-yl)-4,5-dimethyl-5-
(trifluoromethyl)tetrahydrofuran-2-carboxamide

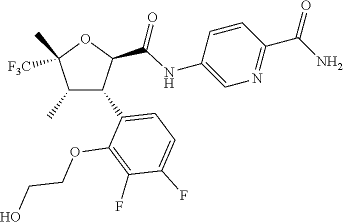 [SMILES: C[C@H]1[C@@H](c2ccc(F)c(F)c2OCCO)[C@H](C(=O)Nc2ccc(C(N)=O)nc2)O[C@@]1(C)C(F)(F)F]

5-((2R,3S,4S,5R)-3-(3,4-difluoro-2-(2-
hydroxyethoxy)phenyl)-4,5-dimethyl-5-
(trifluoromethyl)tetrahydrofuran-2-
carboxamido)picolinamide

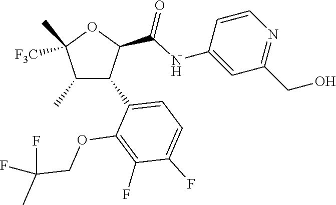 [SMILES: C[C@H]1[C@@H](c2ccc(F)c(F)c2OCC(C)(F)F)[C@H](C(=O)Nc2ccnc(CO)c2)O[C@@]1(C)C(F)(F)F]

(2R,3S,4S,5R)-3-(2-(2,2-difluoropropoxy)-3,4-
difluorophenyl)-N-(2-(hydroxymethyl)pyridin-4-
yl)-4,5-dimethyl-5-
(trifluoromethyl)tetrahydrofuran-2-carboxamide

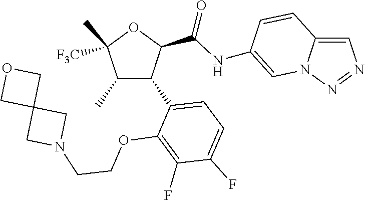 [SMILES: C[C@H]1[C@@H](c2ccc(F)c(F)c2OCCN2CC3(COC3)C2)[C@H](C(=O)Nc2ccc3cnnn3c2)O[C@@]1(C)C(F)(F)F]

(2R,3S,4S,5R)-3-(2-(2-(2-oxa-6-
azaspiro[3.3]heptan-6-yl)ethoxy)-3,4-
difluorophenyl)-N-([1,2,3]triazolo[1,5-a]pyridin-6-
yl)-4,5-dimethyl-5-(trifluoromethyl)tetrahydrofuran-
2-carboxamide

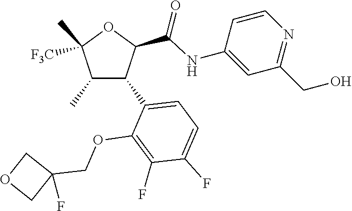 [SMILES: C[C@H]1[C@@H](c2ccc(F)c(F)c2OCC2(F)COC2)[C@H](C(=O)Nc2ccnc(CO)c2)O[C@@]1(C)C(F)(F)F]

(2R,3S,4S,5R)-3-(3,4-difluoro-2-((3-
fluorooxetan-3-yl)methoxy)phenyl)-N-(2-
(hydroxymethyl)pyridin-4-yl)-4,5-dimethyl-5-
(trifluoromethyl)tetrahydrofuran-2-carboxamide

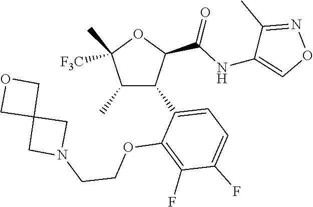 [SMILES: Cc1nocc1NC(=O)[C@@H]1O[C@@](C)(C(F)(F)F)[C@@H](C)[C@H]1c1ccc(F)c(F)c1OCCN1CC2(COC2)C1]

(2R,3S,4S,5R)-3-(2-(2-(2-oxa-6-
azaspiro[3.3]heptan-6-yl)ethoxy)-3,4-
difluorophenyl)-4,5-dimethyl-N-(3-
methylisoxazol-4-yl)-5-
(trifluoromethyl)tetrahydrofuran-2-carboxamide

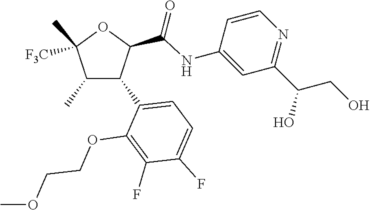 [SMILES: COCCOc1c([C@H]2[C@H](C(=O)Nc3ccnc([C@@H](O)CO)c3)O[C@@](C)(C(F)(F)F)[C@H]2C)ccc(F)c1F]

(2R,3S,4S,5R)-3-(3,4-difluoro-2-(2-
methoxyethoxy)phenyl)-N-(2-((R)-1,2-
dihydroxyethyl)pyridin-4-yl)-4,5-dimethyl-5-
(trifluoromethyl)tetrahydrofuran-2-carboxamide

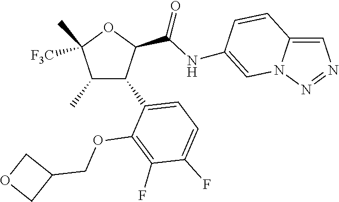 [SMILES: C[C@H]1[C@@H](c2ccc(F)c(F)c2OCC2COC2)[C@H](C(=O)Nc2ccc3cnnn3c2)O[C@@]1(C)C(F)(F)F]

(2R,3S,4S,5R)-N-([1,2,3]triazolo[1,5-
a]pyridin-6-yl)-3-(3,4-difluoro-2-(oxetan-3-
ylmethoxy)phenyl)-4,5-dimethyl-5-
(trifluoromethyl)tetrahydrofuran-2-
carboxamide

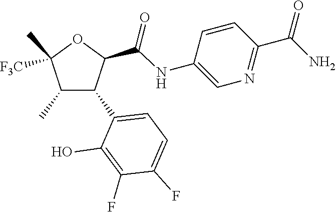 [SMILES: C[C@H]1[C@@H](c2ccc(F)c(F)c2O)[C@H](C(=O)Nc2ccc(C(N)=O)nc2)O[C@@]1(C)C(F)(F)F]

5-((2R,3S,4S,5R)-3-(3,4-difluoro-2-
hydroxyphenyl)-4,5-dimethyl-5-
(trifluoromethyl)tetrahydrofuran-2-
carboxamido)picolinamide

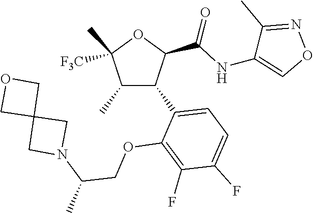 [SMILES: Cc1nocc1NC(=O)[C@@H]1O[C@@](C)(C(F)(F)F)[C@@H](C)[C@H]1c1ccc(F)c(F)c1OC[C@H](C)N1CC2(COC2)C1]

(2R,3S,4S,5R)-3-(2-((S)-2-(2-oxa-6-
azaspiro[3.3]heptan-6-yl)propoxy)-3,4-
difluorophenyl)-4,5-dimethyl-N-(3-methylisoxazol-
4-yl)-5-(trifluoromethyl)tetrahydrofuran-2-
carboxamide

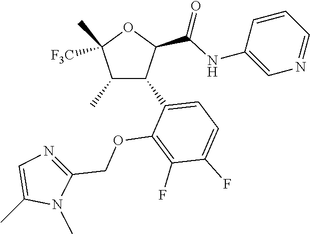 [SMILES: Cc1cnc(COc2c([C@H]3[C@H](C(=O)Nc4cccnc4)O[C@@](C)(C(F)(F)F)[C@H]3C)ccc(F)c2F)n1C]

(2R,3S,4S,5R)-3-(2-((1,5-dimethyl-1H-imidazol-
2-yl)methoxy)-3,4-difluorophenyl)-4,5-dimethyl-
N-(pyridin-3-yl)-5-
(trifluoromethyl)tetrahydrofuran-2-carboxamide

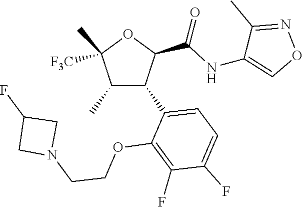 [SMILES: Cc1nocc1NC(=O)[C@@H]1O[C@@](C)(C(F)(F)F)[C@@H](C)[C@H]1c1ccc(F)c(F)c1OCCN1CC(F)C1]

(2R,3S,4S,5R)-3-(3,4-difluoro-2-(2-(3-
fluoroazetidin-1-yl)ethoxy)phenyl)-4,5-dimethyl-
N-(3-methylisoxazol-4-yl)-5-
(trifluoromethyl)tetrahydrofuran-2-carboxamide

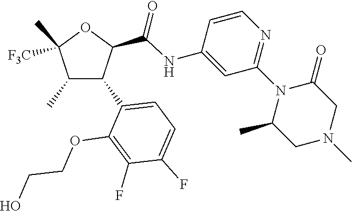 [SMILES: C[C@@H]1CN(C)CC(=O)N1c1cc(NC(=O)[C@@H]2O[C@@](C)(C(F)(F)F)[C@@H](C)[C@H]2c2ccc(F)c(F)c2OCCO)ccn1]

(2R,3S,4S,5R)-3-(3,4-difluoro-2-(2-
hydroxyethoxy)phenyl)-N-(2-((R)-2,4-dimethyl-6-
oxopiperazin-1-yl)pyridin-4-yl)-4,5-dimethyl-5-
(trifluoromethyl)tetrahydrofuran-2-carboxamide

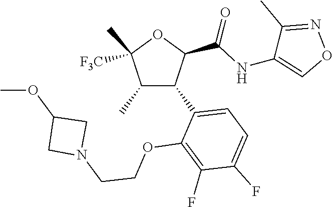 [SMILES: COC1CN(CCOc2c([C@H]3[C@H](C(=O)Nc4conc4C)O[C@@](C)(C(F)(F)F)[C@H]3C)ccc(F)c2F)C1]

(2R,3S,4S,5R)-3-(3,4-difluoro-2-(2-(3-
methoxyazetidin-1-yl)ethoxy)phenyl)-4,5-
dimethyl-N-(3-methylisoxazol-4-yl)-5-
(trifluoromethyl)tetrahydrofuran-2-carboxamide

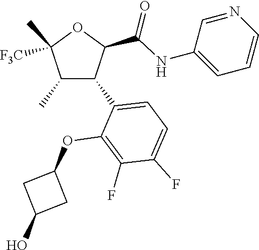 [SMILES: C[C@H]1[C@@H](c2ccc(F)c(F)c2O[C@H]2C[C@@H](O)C2)[C@H](C(=O)Nc2cccnc2)O[C@@]1(C)C(F)(F)F]

(2R,3S,4S,5R)-3-(3,4-difluoro-2-((1s,3R)-3-
hydroxycyclobutoxy)phenyl)-4,5-dimethyl-N-
(pyridin-3-yl)-5-(trifluoromethyl)tetrahydrofuran-
2-carboxamide

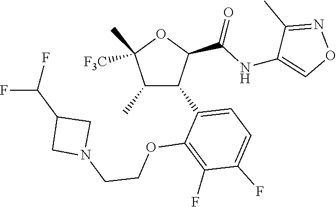 [SMILES: Cc1nocc1NC(=O)[C@@H]1O[C@@](C)(C(F)(F)F)[C@@H](C)[C@H]1c1ccc(F)c(F)c1OCCN1CC(C(F)F)C1]

(2R,3S,4S,5R)-3-(2-(2-(3-
(difluoromethyl)azetidin-1-yl)ethoxy)-3,4-
difluorophenyl)-4,5-dimethyl-N-(3-methylisoxazol-
4-yl)-5-(trifluoromethyl)tetrahydrofuran-2-
carboxamide

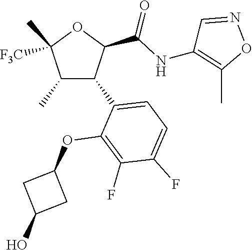 [SMILES: Cc1oncc1NC(=O)[C@@H]1O[C@@](C)(C(F)(F)F)[C@@H](C)[C@H]1c1ccc(F)c(F)c1O[C@H]1C[C@@H](O)C1]

(2R,3S,4S,5R)-3-(3,4-difluoro-2-((1s,3R)-3-
hydroxycyclobutoxy)phenyl)-4,5-dimethyl-N-(5-
methylisoxazol-4-yl)-5-
(trifluoromethyl)tetrahydrofuran-2-carboxamide

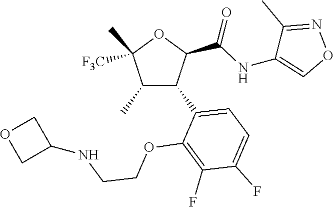 [SMILES: Cc1nocc1NC(=O)[C@@H]1O[C@@](C)(C(F)(F)F)[C@@H](C)[C@H]1c1ccc(F)c(F)c1OCCNC1COC1]

(2R,3S,4S,5R)-3-(3,4-difluoro-2-(2-(oxetan-3-
ylamino)ethoxy)phenyl)-4,5-dimethyl-N-(3-
methylisoxazol-4-yl)-5-
(trifluoromethyl)tetrahydrofuran-2-carboxamide

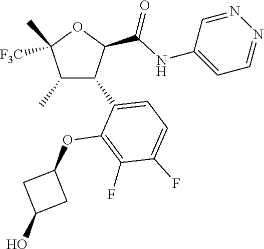 [SMILES: C[C@H]1[C@@H](c2ccc(F)c(F)c2O[C@H]2C[C@@H](O)C2)[C@H](C(=O)Nc2ccnnc2)O[C@@]1(C)C(F)(F)F]

(2R,3S,4S,5R)-3-(3,4-difluoro-2-((1s,3R)-3-
hydroxycyclobutoxy)phenyl)-4,5-dimethyl-N-
(pyridazin-4-yl)-5-
(trifluoromethyl)tetrahydrofuran-2-carboxamide

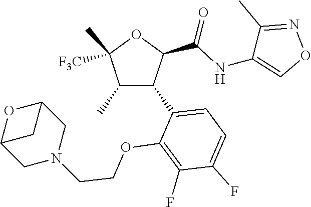 [SMILES: Cc1nocc1NC(=O)[C@@H]1O[C@@](C)(C(F)(F)F)[C@@H](C)[C@H]1c1ccc(F)c(F)c1OCCN1CC2CC(C1)O2]

(2R,3S,4S,5R)-3-(2-(2-(6-oxa-3-
azabicyclo[3.1.1]heptan-3-yl)ethoxy)-3,4-
difluorophenyl)-4,5-dimethyl-N-(3-
methylisoxazol-4-yl)-5-
(trifluoromethyl)tetrahydrofuran-2-carboxamide

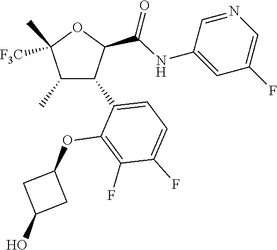 [SMILES: C[C@H]1[C@@H](c2ccc(F)c(F)c2O[C@H]2C[C@@H](O)C2)[C@H](C(=O)Nc2cncc(F)c2)O[C@@]1(C)C(F)(F)F]

(2R,3S,4S,5R)-3-(3,4-difluoro-2-((1s,3R)-3-
hydroxycyclobutoxy)phenyl)-N-(5-fluoropyridin-
3-yl)-4,5-dimethyl-5-
(trifluoromethyl)tetrahydrofuran-2-carboxamide

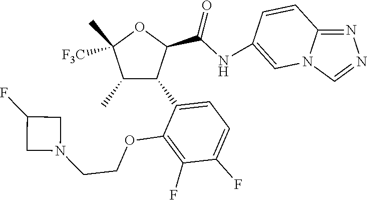 [SMILES: C[C@H]1[C@@H](c2ccc(F)c(F)c2OCCN2CC(F)C2)[C@H](C(=O)Nc2ccc3nncn3c2)O[C@@]1(C)C(F)(F)F]

(2R,3S,4S,5R)-N-([1,2,4]triazolo[4,3-a]pyridin-6-yl)-
3-(3,4-difluoro-2-(2-(3-fluoroazetidin-1-
yl)ethoxy)phenyl)-4,5-dimethyl-5-
(trifluoromethyl)tetrahydrofuran-2-carboxamide

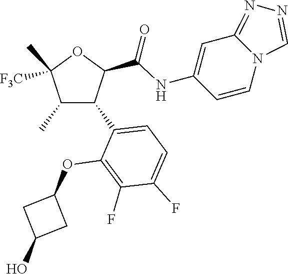 [SMILES: C[C@H]1[C@@H](c2ccc(F)c(F)c2O[C@H]2C[C@@H](O)C2)[C@H](C(=O)Nc2ccn3cnnc3c2)O[C@@]1(C)C(F)(F)F]

(2R,3S,4S,5R)-N-([1,2,4]triazolo[4,3-a]pyridin-7-
yl)-3-(3,4-difluoro-2-((1s,3R)-3-
hydroxycyclobutoxy)phenyl)-4,5-dimethyl-5-
(trifluoromethyl)tetrahydrofuran-2-carboxamide

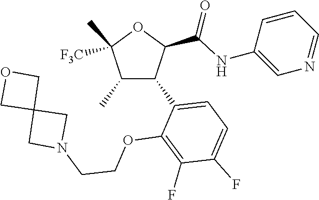 [SMILES: C[C@H]1[C@@H](c2ccc(F)c(F)c2OCCN2CC3(COC3)C2)[C@H](C(=O)Nc2cccnc2)O[C@@]1(C)C(F)(F)F]

(2R,3S,4S,5R)-3-(2-(2-(2-oxa-6-
azaspiro[3.3]heptan-6-yl)ethoxy)-3,4-
difluorophenyl)-4,5-dimethyl-N-(pyridin-3-yl)-5-
(trifluoromethyl)tetrahydrofuran-2-carboxamide

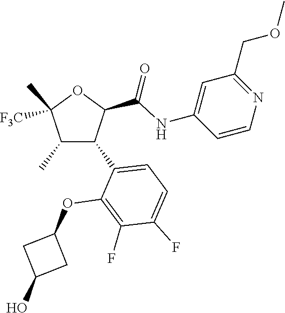 [SMILES: COCc1cc(NC(=O)[C@@H]2O[C@@](C)(C(F)(F)F)[C@@H](C)[C@H]2c2ccc(F)c(F)c2O[C@H]2C[C@@H](O)C2)ccn1]

(2R,3S,4S,5R)-3-(3,4-difluoro-2-((1s,3R)-3-
hydroxycyclobutoxy)phenyl)-N-(2-
(methoxymethyl)pyridin-4-yl)-4,5-dimethyl-5-
(trifluoromethyl)tetrahydrofuran-2-carboxamide

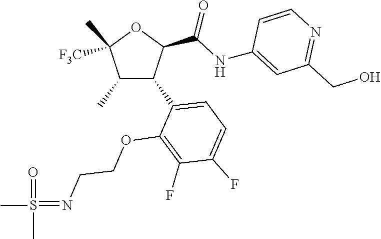 [SMILES: C[C@H]1[C@@H](c2ccc(F)c(F)c2OCCN=S(C)(C)=O)[C@H](C(=O)Nc2ccnc(CO)c2)O[C@@]1(C)C(F)(F)F]

(2R,3S,4S,5R)-3-(2-(2-((dimethyl(oxo)-λ⁶-
sulfaneylidene)amino)ethoxy)-3,4-
difluorophenyl)-N-(2-(hydroxymethyl)pyridin-4-
yl)-4,5-dimethyl-5-
(trifluoromethyl)tetrahydrofuran-2-carboxamide

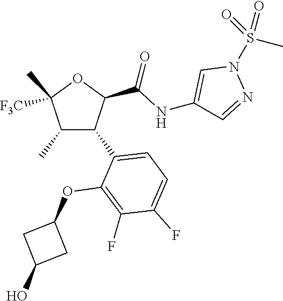 [SMILES: C[C@H]1[C@@H](c2ccc(F)c(F)c2O[C@H]2C[C@@H](O)C2)[C@H](C(=O)Nc2cnn(S(C)(=O)=O)c2)O[C@@]1(C)C(F)(F)F]

(2R,3S,4S,5R)-3-(3,4-difluoro-2-((1s,3R)-3-
hydroxycyclobutoxy)phenyl)-4,5-dimethyl-N-(1-
(methylsulfonyl)-1H-pyrazol-4-yl)-5-
(trifluoromethyl)tetrahydrofuran-2-carboxamide

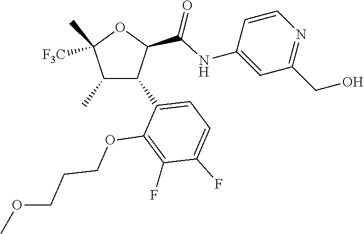 [SMILES: COCCCOc1c([C@H]2[C@H](C(=O)Nc3ccnc(CO)c3)O[C@@](C)(C(F)(F)F)[C@H]2C)ccc(F)c1F]

(2R,3S,4S,5R)-3-(3,4-difluoro-2-(3-
methoxypropoxy)phenyl)-N-(2-
(hydroxymethyl)pyridin-4-yl)-4,5-dimethyl-5-
(trifluoromethyl)tetrahydrofuran-2-carboxamide

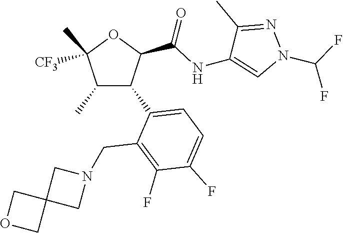 [SMILES: Cc1nn(C(F)F)cc1NC(=O)[C@@H]1O[C@@](C)(C(F)(F)F)[C@@H](C)[C@H]1c1ccc(F)c(F)c1CN1CC2(COC2)C1]

(2R,3S,4S,5R)-3-(2-((2-oxa-6-
azaspiro[3.3]heptan-6-yl)methyl)-3,4-
difluorophenyl)-N-(1-(difluoromethyl)-3-methyl-
1H-pyrazol-4-yl)-4,5-dimethyl-5-
(trifluoromethyl)tetrahydrofuran-2-carboxamide

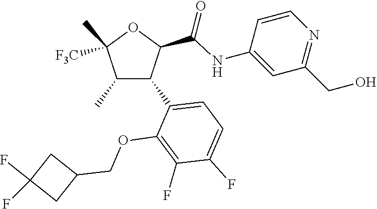 [SMILES: C[C@H]1[C@@H](c2ccc(F)c(F)c2OCC2CC(F)(F)C2)[C@H](C(=O)Nc2ccnc(CO)c2)O[C@@]1(C)C(F)(F)F]

(2R,3S,4S,5R)-3-(2-((3,3-
difluorocyclobutyl)methoxy)-3,4-difluorophenyl)-N-(2-
(hydroxymethyl)pyridin-4-yl)-4,5-dimethyl-5-
(trifluoromethyl)tetrahydrofuran-2-carboxamide

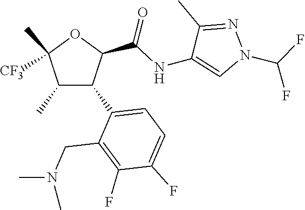 [SMILES: Cc1nn(C(F)F)cc1NC(=O)[C@@H]1O[C@@](C)(C(F)(F)F)[C@@H](C)[C@H]1c1ccc(F)c(F)c1CN(C)C]

(2R,3S,4S,5R)-N-(1-(difluoromethyl)-3-methyl-
1H-pyrazol-4-yl)-3-(2-((dimethylamino)methyl)-
3,4-difluorophenyl)-4,5-dimethyl-5-
(trifluoromethyl)tetrahydrofuran-2-carboxamide

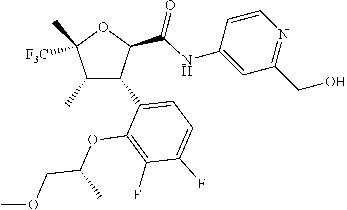 [SMILES: COC[C@@H](C)Oc1c([C@H]2[C@H](C(=O)Nc3ccnc(CO)c3)O[C@@](C)(C(F)(F)F)[C@H]2C)ccc(F)c1F]

(2R,3S,4S,5R)-3-(3,4-difluoro-2-(((R)-1-
methoxypropan-2-yl)oxy)phenyl)-N-(2-
(hydroxymethyl)pyridin-4-yl)-4,5-dimethyl-5-
(trifluoromethyl)tetrahydrofuran-2-carboxamide

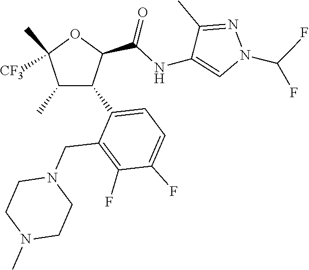

(2R,3S,4S,5R)-3-(3,4-difluoro-2-((4-
methylpiperazin-1-yl)methyl)phenyl)-N-(1-
(difluoromethyl)-3-methyl-1H-pyrazol-4-yl)-4,5-
dimethyl-5-(trifluoromethyl)tetrahydrofuran-2-
carboxamide

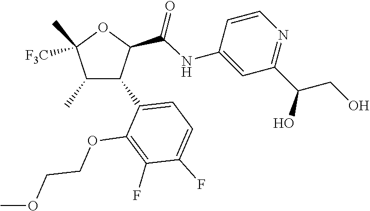 [SMILES: COCCOc1c([C@H]2[C@H](C(=O)Nc3ccnc([C@H](O)CO)c3)O[C@@](C)(C(F)(F)F)[C@H]2C)ccc(F)c1F]

(2R,3S,4S,5R)-3-(3,4-difluoro-2-(2-
methoxyethoxy)phenyl)-N-(2-((R)-1,2-
dihydroxyethyl)pyridin-4-yl)-4,5-dimethyl-5-
(trifluoromethyl)tetrahydrofuran-2-carboxamide

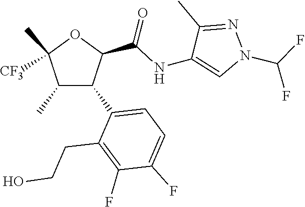 [SMILES: Cc1nn(C(F)F)cc1NC(=O)[C@@H]1O[C@@](C)(C(F)(F)F)[C@@H](C)[C@H]1c1ccc(F)c(F)c1CCO]

(2R,3S,4S,5R)-3-(3,4-difluoro-2-(2-
hydroxyethyl)phenyl)-N-(1-(difluoromethyl)-3-
methyl-1H-pyrazol-4-yl)-4,5-dimethyl-5-
(trifluoromethyl)tetrahydrofuran-2-carboxamide

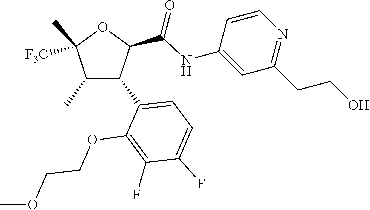 [SMILES: COCCOc1c([C@H]2[C@H](C(=O)Nc3ccnc(CCO)c3)O[C@@](C)(C(F)(F)F)[C@H]2C)ccc(F)c1F]

(2R,3S,4S,5R)-3-(3,4-difluoro-2-(2-
methoxyethoxy)phenyl)-N-(2-(2-
hydroxyethyl)pyridin-4-yl)-4,5-dimethyl-5-
(trifluoromethyl)tetrahydrofuran-2-carboxamide

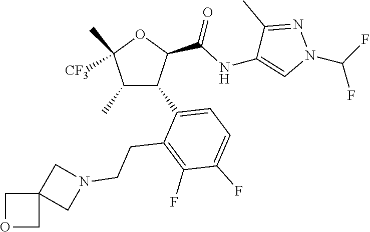 [SMILES: Cc1nn(C(F)F)cc1NC(=O)[C@@H]1O[C@@](C)(C(F)(F)F)[C@@H](C)[C@H]1c1ccc(F)c(F)c1CCN1CC2(COC2)C1]

(2R,3S,4S,5R)-3-(2-(2-(2-oxa-6-azaspiro[3.3]heptan-6-
yl)ethyl)-3,4-difluorophenyl)-N-(1-(difluoromethyl)-3-
methyl-1H-pyrazol-4-yl)-4,5-dimethyl-5-
(trifluoromethyl)tetrahydrofuran-2-carboxamide

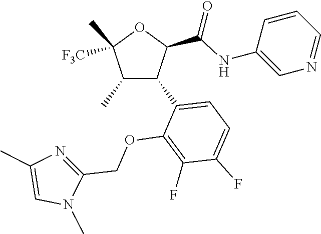 [SMILES: Cc1cn(C)c(COc2c([C@H]3[C@H](C(=O)Nc4cccnc4)O[C@@](C)(C(F)(F)F)[C@H]3C)ccc(F)c2F)n1]

(2R,3S,4S,5R)-3-(2-((1,4-dimethyl-1H-imidazol-
2-yl)methoxy)-3,4-difluorophenyl)-4,5-dimethyl-
N-(pyridin-3-yl)-5-
(trifluoromethyl)tetrahydrofuran-2-carboxamide

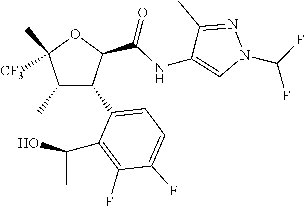 [SMILES: Cc1nn(C(F)F)cc1NC(=O)[C@@H]1O[C@@](C)(C(F)(F)F)[C@@H](C)[C@H]1c1ccc(F)c(F)c1[C@@H](C)O]

(2R,3S,4S,5R)-3-
(3,4-difluoro-2-(1-
hydroxyethyl)phenyl)-
N-(1-
(difluoromethyl)-3-
methyl-1H-pyrazol-
4-yl)-4,5-dimethyl-5-
(trifluoromethyl)tetra-
hydrofuran-2-
carboxamide

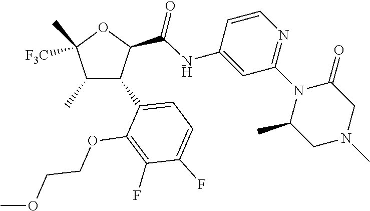

(2R,3S,4S,5R)-3-(3,4-difluoro-2-(2-
methoxyethoxy)phenyl)-N-(2-((R)-2,4-dimethyl-6-
oxopiperazin-1-yl)pyridin-4-yl)-4,5-dimethyl-5-
(trifluoromethyl)tetrahydrofuran-2-carboxamide

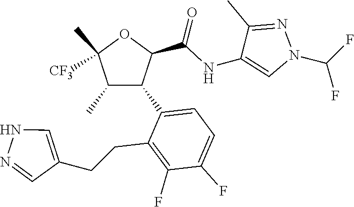 [SMILES: Cc1nn(C(F)F)cc1NC(=O)[C@@H]1O[C@@](C)(C(F)(F)F)[C@@H](C)[C@H]1c1ccc(F)c(F)c1CCc1cn[nH]c1]

(2R,3S,4S,5R)-3-(2-(2-(1H-pyrazol-4-yl)ethyl)-3,4-
difluorophenyl)-N-(1-(difluoromethyl)-3-methyl-1H-
pyrazol-4-yl)-4,5-dimethyl-5-
(trifluoromethyl)tetrahydrofuran-2-carboxamide

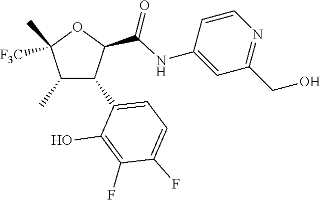 [SMILES: C[C@H]1[C@@H](c2ccc(F)c(F)c2O)[C@H](C(=O)Nc2ccnc(CO)c2)O[C@@]1(C)C(F)(F)F]

(2R,3S,4S,5R)-3-(3,4-difluoro-2-
hydroxyphenyl)-N-(2-(hydroxymethyl)pyridin-
4-yl)-4,5-dimethyl-5-
(trifluoromethyl)tetrahydrofuran-2-
carboxamide

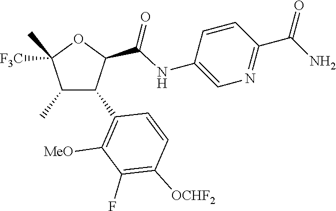 [SMILES: COc1c([C@H]2[C@H](C(=O)Nc3ccc(C(N)=O)nc3)O[C@@](C)(C(F)(F)F)[C@H]2C)ccc(OC(F)F)c1F]

5-((2R,3S,4S,5R)-3-(4-(difluoromethoxy)-3-
fluoro-2-methoxyphenyl)-4,5-dimethyl-5-
(trifluoromethyl)tetrahydrofuran-2-
carboxamido)picolinamide

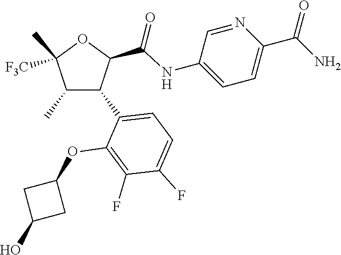 [SMILES: C[C@H]1[C@@H](c2ccc(F)c(F)c2O[C@H]2C[C@@H](O)C2)[C@H](C(=O)Nc2ccc(C(N)=O)nc2)O[C@@]1(C)C(F)(F)F]

5-((2R,3S,4S,5R)-3-(3,4-difluoro-2-((1s,3R)-3-
hydroxycyclobutoxy)phenyl)-4,5-dimethyl-5-
(trifluoromethyl)tetrahydrofuran-2-
carboxamido)picolinamide

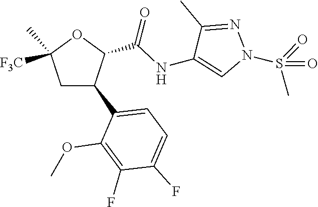 [SMILES: COc1c([C@H]2C[C@@](C)(C(F)(F)F)O[C@@H]2C(=O)Nc2cn(S(C)(=O)=O)nc2C)ccc(F)c1F]

(2S,3R,5S)-3-(3,4-difluoro-2-methoxyphenyl)-5-
methyl-N-(3-methyl-1-(methylsulfonyl)-1H-
pyrazol-4-yl)-5-(trifluoromethyl)tetrahydrofuran-
2-carboxamide

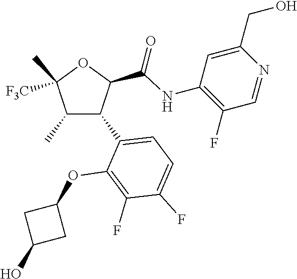 [SMILES: C[C@H]1[C@@H](c2ccc(F)c(F)c2O[C@H]2C[C@@H](O)C2)[C@H](C(=O)Nc2cc(CO)ncc2F)O[C@@]1(C)C(F)(F)F]

(2R,3S,4S,5R)-3-(3,4-difluoro-2-((1s,3R)-3-
hydroxycyclobutoxy)phenyl)-N-(5-fluoro-2-
(hydroxymethyl)pyridin-4-yl)-4,5-dimethyl-5-
(trifluoromethyl)tetrahydrofuran-2-carboxamide

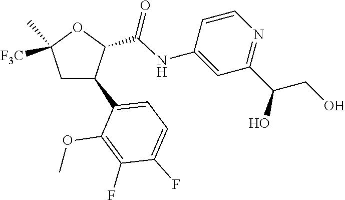 [SMILES: COc1c([C@H]2C[C@@](C)(C(F)(F)F)O[C@@H]2C(=O)Nc2ccnc([C@H](O)CO)c2)ccc(F)c1F]

(2S,3R,5S)-3-(3,4-difluoro-2-methoxyphenyl)-N-
(2-((S)-1,2-dihydroxyethyl)pyridin-4-yl)-5-methyl-
5-(trifluoromethyl)tetrahydrofuran-2-carboxamide

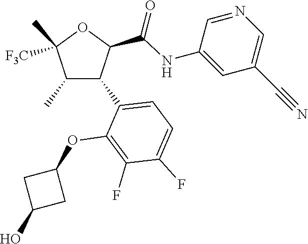 [SMILES: C[C@H]1[C@@H](c2ccc(F)c(F)c2O[C@H]2C[C@@H](O)C2)[C@H](C(=O)Nc2cncc(C#N)c2)O[C@@]1(C)C(F)(F)F]

(2R,3S,4S,5R)-N-(5-cyanopyridin-3-yl)-3-(3,4-
difluoro-2-((1s,3R)-3-
hydroxycyclobutoxy)phenyl)-4,5-dimethyl-5-
(trifluoromethyl)tetrahydrofuran-2-carboxamide

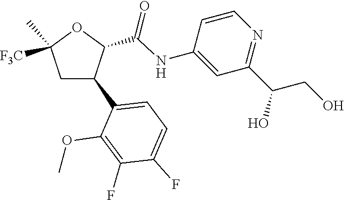 [SMILES: COc1c([C@H]2C[C@@](C)(C(F)(F)F)O[C@@H]2C(=O)Nc2ccnc([C@@H](O)CO)c2)ccc(F)c1F]

(2S,3R,5S)-3-(3,4-difluoro-2-methoxyphenyl)-N-
(2-((R)-1,2-dihydroxyethyl)pyridin-4-yl)-5-
methyl-5-(trifluoromethyl)tetrahydrofuran-2-
carboxamide

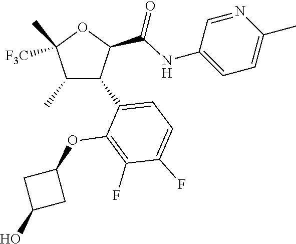 [SMILES: Cc1ccc(NC(=O)[C@@H]2O[C@@](C)(C(F)(F)F)[C@@H](C)[C@H]2c2ccc(F)c(F)c2O[C@H]2C[C@@H](O)C2)cn1]

(2R,3S,4S,5R)-3-(3,4-difluoro-2-((1s,3R)-3-
hydroxycyclobutoxy)phenyl)-4,5-dimethyl-N-(6-
methylpyridin-3-yl)-5-
(trifluoromethyl)tetrahydrofuran-2-carboxamide

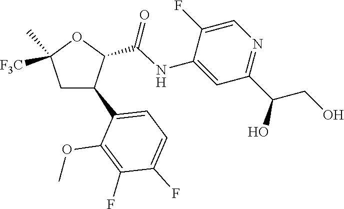 [SMILES: COc1c([C@H]2C[C@@](C)(C(F)(F)F)O[C@@H]2C(=O)Nc2cc([C@H](O)CO)ncc2F)ccc(F)c1F]

(2S,3R,5S)-3-(3,4-difluoro-2-methoxyphenyl)-N-
(2-((S)-1,2-dihydroxyethyl)-5-fluoropyridin-4-yl)-
5-methyl-5-(trifluoromethyl)tetrahydrofuran-2-
carboxamide

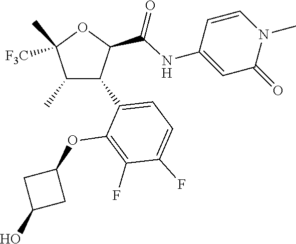 [SMILES: C[C@H]1[C@@H](c2ccc(F)c(F)c2O[C@H]2C[C@@H](O)C2)[C@H](C(=O)Nc2ccn(C)c(=O)c2)O[C@@]1(C)C(F)(F)F]

(2R,3S,4S,5R)-3-(3,4-difluoro-2-((1s,3R)-3-
hydroxycyclobutoxy)phenyl)-4,5-dimethyl-N-(1-
methyl-2-oxo-1,2-dihydropyridin-4-yl)-5-
(trifluoromethyl)tetrahydrofuran-2-carboxamide

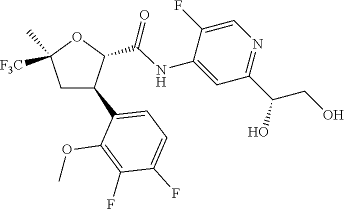 [SMILES: COc1c([C@H]2C[C@@](C)(C(F)(F)F)O[C@@H]2C(=O)Nc2cc([C@@H](O)CO)ncc2F)ccc(F)c1F]

(2S,3R,5S)-3-(3,4-difluoro-2-methoxyphenyl)-N-
(2-((R)-1,2-dihydroxyethyl)-5-fluoropyridin-4-yl)-
5-methyl-5-(trifluoromethyl)tetrahydrofuran-2-
carboxamide

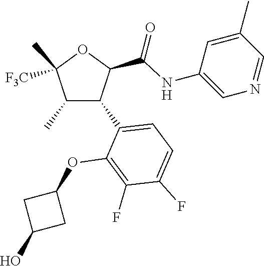 [SMILES: Cc1cncc(NC(=O)[C@@H]2O[C@@](C)(C(F)(F)F)[C@@H](C)[C@H]2c2ccc(F)c(F)c2O[C@H]2C[C@@H](O)C2)c1]

(2R,3S,4S,5R)-3-(3,4-difluoro-2-((1s,3R)-3-
hydroxycyclobutoxy)phenyl)-4,5-dimethyl-N-(5-
methylpyridin-3-yl)-5-
(trifluoromethyl)tetrahydrofuran-2-carboxamide

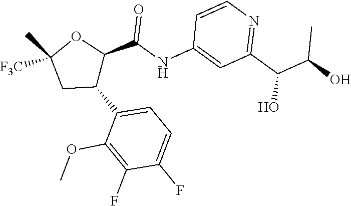 [SMILES: COc1c([C@@H]2C[C@](C)(C(F)(F)F)O[C@H]2C(=O)Nc2ccnc([C@@H](O)[C@@H](C)O)c2)ccc(F)c1F]

(2R,3S,5R)-3-(3,4-difluoro-2-methoxyphenyl)-N-
(2-((1R,2R)-1,2-dihydroxypropyl)pyridin-4-yl)-5-
methyl-5-(trifluoromethyl)tetrahydrofuran-2-
carboxamide

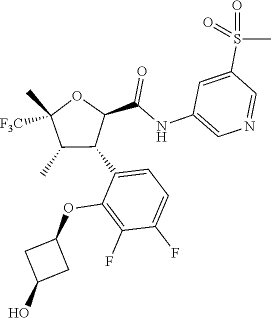 [SMILES: C[C@H]1[C@@H](c2ccc(F)c(F)c2O[C@H]2C[C@@H](O)C2)[C@H](C(=O)Nc2cncc(S(C)(=O)=O)c2)O[C@@]1(C)C(F)(F)F]

(2R,3S,4S,5R)-3-(3,4-difluoro-2-((1s,3R)-3-
hydroxycyclobutoxy)phenyl)-4,5-dimethyl-N-(5-
(methylsulfonyl)pyridin-3-yl)-5-
(trifluoromethyl)tetrahydrofuran-2-carboxamide

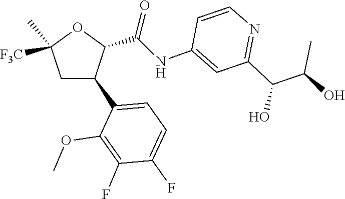 [SMILES: COc1c([C@H]2C[C@@](C)(C(F)(F)F)O[C@@H]2C(=O)Nc2ccnc([C@@H](O)[C@@H](C)O)c2)ccc(F)c1F]

(2S,3R,5S)-3-(3,4-difluoro-2-methoxyphenyl)-N-
(2-((1R,2R)-1,2-dihydroxypropyl)pyridin-4-yl)-5-
methyl-5-(trifluoromethyl)tetrahydrofuran-2-
carboxamide

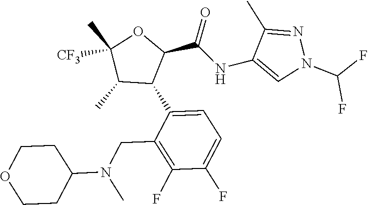 [SMILES: Cc1nn(C(F)F)cc1NC(=O)[C@@H]1O[C@@](C)(C(F)(F)F)[C@@H](C)[C@H]1c1ccc(F)c(F)c1CN(C)C1CCOCC1]

(2R,3S,4S,5R)-3-(3,4-difluoro-2-
((methyl(tetrahydro-2H-pyran-4-
yl)amino)methyl)phenyl)-N-(1-(difluoromethyl)-3-
methyl-1H-pyrazol-4-yl)-4,5-dimethyl-5-
(trifluoromethyl)tetrahydrofuran-2-carboxamide

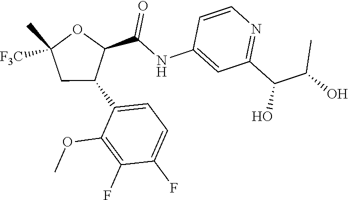 [SMILES: COc1c([C@@H]2C[C@](C)(C(F)(F)F)O[C@H]2C(=O)Nc2ccnc([C@@H](O)[C@H](C)O)c2)ccc(F)c1F]

(2R,3S,5R)-3-(3,4-difluoro-2-methoxyphenyl)-N-
(2-((1R,2S)-1,2-dihydroxypropyl)pyridin-4-yl)-5-
methyl-5-(trifluoromethyl)tetrahydrofuran-2-
carboxamide

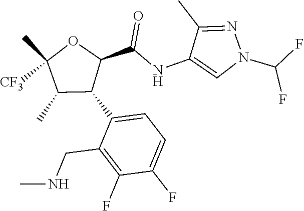 [SMILES: CNCc1c([C@H]2[C@H](C(=O)Nc3cn(C(F)F)nc3C)O[C@@](C)(C(F)(F)F)[C@H]2C)ccc(F)c1F]

(2R,3S,4S,5R)-3-(3,4-difluoro-2-
((methylamino)methyl)phenyl)-N-(1-
(difluoromethyl)-3-methyl-1H-pyrazol-4-yl)-4,5-
dimethyl-5-(trifluoromethyl)tetrahydrofuran-2-
carboxamide

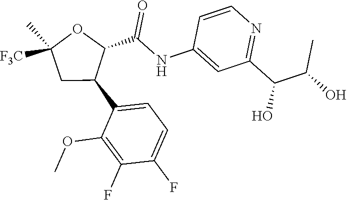 [SMILES: COc1c([C@H]2C[C@@](C)(C(F)(F)F)O[C@@H]2C(=O)Nc2ccnc([C@@H](O)[C@H](C)O)c2)ccc(F)c1F]

(2S,3R,5S)-3-(3,4-difluoro-2-methoxyphenyl)-N-
(2-((1R,2S)-1,2-dihydroxypropyl)pyridin-4-yl)-5-
methyl-5-(trifluoromethyl)tetrahydrofuran-2-
carboxamide

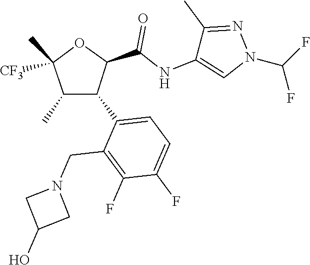

(2R,3S,4S,5R)-3-(3,4-difluoro-2-((3-
hydroxyazetidin-1-yl)methyl)phenyl)-N-(1-
(difluoromethyl)-3-methyl-1H-pyrazol-4-yl)-4,5-
dimethyl-5-(trifluoromethyl)tetrahydrofuran-2-
carboxamide

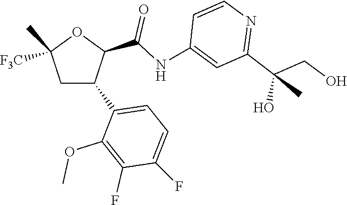 [SMILES: COc1c([C@@H]2C[C@](C)(C(F)(F)F)O[C@H]2C(=O)Nc2ccnc([C@@](C)(O)CO)c2)ccc(F)c1F]

(2R,3S,5R)-3-(3,4-difluoro-2-methoxyphenyl)-N-
(2-((R)-1,2-dihydroxypropan-2-yl)pyridin-4-yl)-5-
methyl-5-(trifluoromethyl)tetrahydrofuran-2-
carboxamide

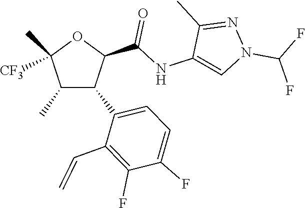 [SMILES: C=Cc1c([C@H]2[C@H](C(=O)Nc3cn(C(F)F)nc3C)O[C@@](C)(C(F)(F)F)[C@H]2C)ccc(F)c1F]

(2R,3S,4S,5R)-3-(3,4-difluoro-2-vinylphenyl)-N-
(1-(difluoromethyl)-3-methyl-1H-pyrazol-4-yl)-
4,5-dimethyl-5-(trifluoromethyl)tetrahydrofuran-
2-carboxamide

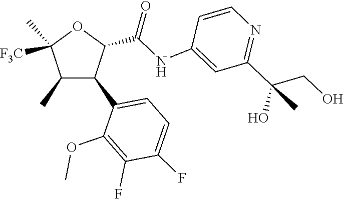 [SMILES: COc1c([C@@H]2[C@@H](C(=O)Nc3ccnc([C@@](C)(O)CO)c3)O[C@](C)(C(F)(F)F)[C@@H]2C)ccc(F)c1F]

(2S,3R,4R,5S)-3-(3,4-difluoro-2-methoxyphenyl)-
N-(2-((R)-1,2-dihydroxypropan-2-yl)pyridin-4-yl)-
4,5-dimethyl-5-(trifluoromethyl)tetrahydrofuran-2-
carboxamide

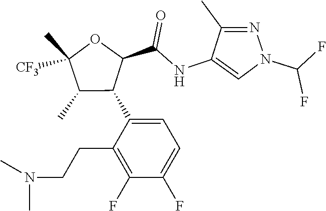 [SMILES: Cc1nn(C(F)F)cc1NC(=O)[C@@H]1O[C@@](C)(C(F)(F)F)[C@@H](C)[C@H]1c1ccc(F)c(F)c1CCN(C)C]

(2R,3S,4S,5R)-N-(1-(difluoromethyl)-3-methyl-1H-
pyrazol-4-yl)-3-(2-(2-(dimethylamino)ethyl)-3,4-
difluorophenyl)-4,5-dimethyl-5-
(trifluoromethyl)tetrahydrofuran-2-carboxamide

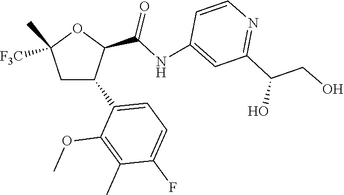 [SMILES: COc1c([C@@H]2C[C@](C)(C(F)(F)F)O[C@H]2C(=O)Nc2ccnc([C@@H](O)CO)c2)ccc(F)c1C]

(2R,3S,5R)-N-(2-((R)-1,2-dihydroxyethyl)pyridin-
4-yl)-3-(4-fluoro-2-methoxy-3-methylphenyl)-5-
methyl-5-(trifluoromethyl)tetrahydrofuran-2-
carboxamide

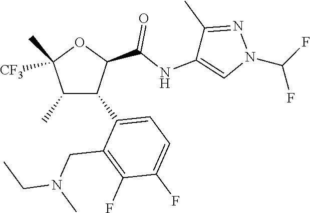 [SMILES: CCN(C)Cc1c([C@H]2[C@H](C(=O)Nc3cn(C(F)F)nc3C)O[C@@](C)(C(F)(F)F)[C@H]2C)ccc(F)c1F]

(2R,3S,4S,5R)-N-(1-(difluoromethyl)-3-methyl-1H-
pyrazol-4-yl)-3-(2-((ethyl(methyl)amino)methyl)-3,4-
difluorophenyl)-4,5-dimethyl-5-
(trifluoromethyl)tetrahydrofuran-2-carboxamide

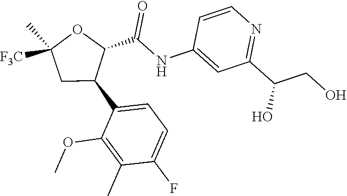 [SMILES: COc1c([C@H]2C[C@@](C)(C(F)(F)F)O[C@@H]2C(=O)Nc2ccnc([C@@H](O)CO)c2)ccc(F)c1C]

(2S,3R,5S)-N-(2-((R)-1,2-
dihydroxyethyl)pyridin-4-yl)-3-(4-fluoro-2-
methoxy-3-methylphenyl)-5-methyl-5-
(trifluoromethyl)tetrahydrofuran-2-carboxamide

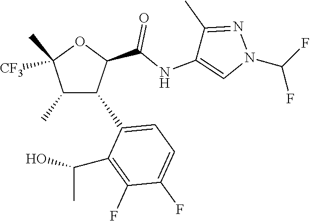 [SMILES: Cc1nn(C(F)F)cc1NC(=O)[C@@H]1O[C@@](C)(C(F)(F)F)[C@@H](C)[C@H]1c1ccc(F)c(F)c1[C@H](C)O]

(2R,3S,4S,5R)-3-
(3,4-difluoro-2-(1-
hydroxyethyl)phenyl)-
N-(1-
(difluoromethyl)-3-
methyl-1H-pyrazol-
4-yl)-4,5-dimethyl-5-
(trifluoromethyl)tetra-
hydrofuran-2-
carboxamide

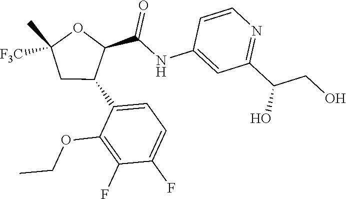 [SMILES: CCOc1c([C@@H]2C[C@](C)(C(F)(F)F)O[C@H]2C(=O)Nc2ccnc([C@@H](O)CO)c2)ccc(F)c1F]

(2R,3S,5R)-N-(2-((R)-1,2-dihydroxyethyl)pyridin-
4-yl)-3-(2-ethoxy-3,4-difluorophenyl)-5-methyl-5-
(trifluoromethyl)tetrahydrofuran-2-carboxamide

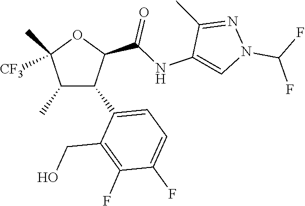 [SMILES: Cc1nn(C(F)F)cc1NC(=O)[C@@H]1O[C@@](C)(C(F)(F)F)[C@@H](C)[C@H]1c1ccc(F)c(F)c1CO]

(2R,3S,4S,5R)-3-(3,4-difluoro-2-
(hydroxymethyl)phenyl)-N-(1-(difluoromethyl)-3-
methyl-1H-pyrazol-4-yl)-4,5-dimethyl-5-
(trifluoromethyl)tetrahydrofuran-2-carboxamide

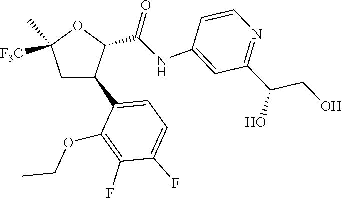 [SMILES: CCOc1c([C@H]2C[C@@](C)(C(F)(F)F)O[C@@H]2C(=O)Nc2ccnc([C@@H](O)CO)c2)ccc(F)c1F]

(2S,3R,5S)-N-(2-((R)-1,2-
dihydroxyethyl)pyridin-4-yl)-3-(2-ethoxy-3,4-
difluorophenyl)-5-methyl-5-
(trifluoromethyl)tetrahydrofuran-2-carboxamide

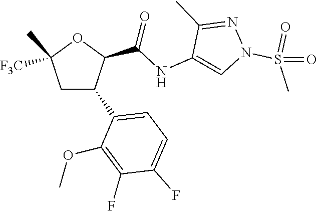 [SMILES: COc1c([C@@H]2C[C@](C)(C(F)(F)F)O[C@H]2C(=O)Nc2cn(S(C)(=O)=O)nc2C)ccc(F)c1F]

(2R,3S,5R)-3-(3,4-difluoro-2-methoxyphenyl)-5-
methyl-N-(3-methyl-1-(methylsulfonyl)-1H-
pyrazol-4-yl)-5-(trifluoromethyl)tetrahydrofuran-
2-carboxamide

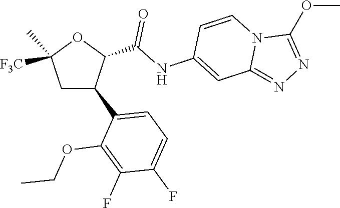 [SMILES: CCOc1c([C@H]2C[C@@](C)(C(F)(F)F)O[C@@H]2C(=O)Nc2ccn3c(OC)nnc3c2)ccc(F)c1F]

(2S,3R,5S)-3-(2-ethoxy-3,4-difluorophenyl)-N-
(3-methoxy-[1,2,4]triazolo[4,3-a]pyridin-7-yl)-5-
methyl-5-(trifluoromethyl)tetrahydrofuran-2-
carboxamide

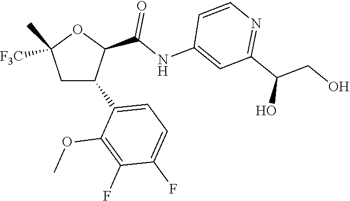 [SMILES: COc1c([C@@H]2C[C@](C)(C(F)(F)F)O[C@H]2C(=O)Nc2ccnc([C@H](O)CO)c2)ccc(F)c1F]

(2R,3S,5R)-3-(3,4-difluoro-2-methoxyphenyl)-N-
(2-((S)-1,2-dihydroxyethyl)pyridin-4-yl)-5-
methyl-5-(trifluoromethyl)tetrahydrofuran-2-
carboxamide

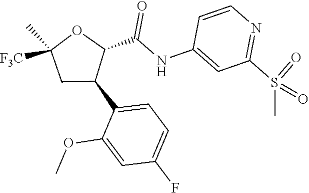 [SMILES: COc1cc(F)ccc1[C@H]1C[C@@](C)(C(F)(F)F)O[C@@H]1C(=O)Nc1ccnc(S(C)(=O)=O)c1]

(2S,3R,5S)-3-(4-fluoro-2-methoxyphenyl)-5-
methyl-N-(2-(methylsulfonyl)pyridin-4-yl)-5-
(trifluoromethyl)tetrahydrofuran-2-carboxamide

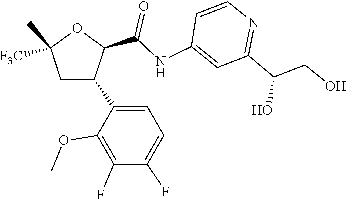 [SMILES: COc1c([C@@H]2C[C@](C)(C(F)(F)F)O[C@H]2C(=O)Nc2ccnc([C@@H](O)CO)c2)ccc(F)c1F]

(2R,3S,5R)-3-(3,4-difluoro-2-methoxyphenyl)-N-
(2-((R)-1,2-dihydroxyethyl)pyridin-4-yl)-5-methyl-
5-(trifluoromethyl)tetrahydrofuran-2-carboxamide

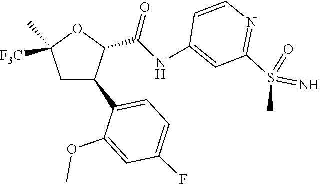 [SMILES: COc1cc(F)ccc1[C@H]1C[C@@](C)(C(F)(F)F)O[C@@H]1C(=O)Nc1ccnc([S@@](C)(=N)=O)c1]

(2S,3R,5S)-3-(4-fluoro-2-methoxyphenyl)-5-
methyl-N-(2-(S-methylsulfonimidoyl)pyridin-4-yl)-
5-(trifluoromethyl)tetrahydrofuran-2-carboxamide

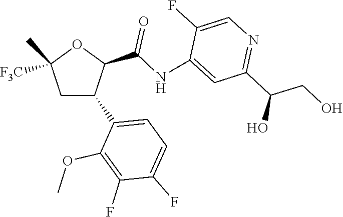 [SMILES: COc1c([C@@H]2C[C@](C)(C(F)(F)F)O[C@H]2C(=O)Nc2cc([C@H](O)CO)ncc2F)ccc(F)c1F]

(2R,3S,5R)-3-(3,4-difluoro-2-methoxyphenyl)-N-
(2-((S)-1,2-dihydroxyethyl)-5-fluoropyridin-4-yl)-
5-methyl-5-(trifluoromethyl)tetrahydrofuran-2-
carboxamide

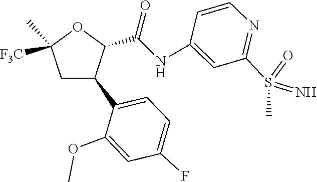 [SMILES: COc1cc(F)ccc1[C@H]1C[C@@](C)(C(F)(F)F)O[C@@H]1C(=O)Nc1ccnc([S@](C)(=N)=O)c1]

(2S,3R,5S)-3-(4-fluoro-2-methoxyphenyl)-5-
methyl-N-(2-(S-methylsulfonimidoyl)pyridin-4-yl)-
5-(trifluoromethyl)tetrahydrofuran-2-carboxamide

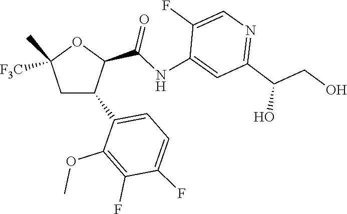 [SMILES: COc1c([C@@H]2C[C@](C)(C(F)(F)F)O[C@H]2C(=O)Nc2cc([C@@H](O)CO)ncc2F)ccc(F)c1F]

(2R,3S,5R)-3-(3,4-difluoro-2-methoxyphenyl)-
N-(2-((R)-1,2-dihydroxyethyl)-5-fluoropyridin-4-
yl)-5-methyl-5-(trifluoromethyl)tetrahydrofuran-
2-carboxamide

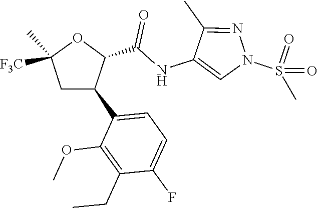 [SMILES: CCc1c(F)ccc([C@H]2C[C@@](C)(C(F)(F)F)O[C@@H]2C(=O)Nc2cn(S(C)(=O)=O)nc2C)c1OC]

(2S,3R,5S)-3-(3-ethyl-4-fluoro-2-
methoxyphenyl)-5-methyl-N-(3-methyl-1-
(methylsulfonyl)-1H-pyrazol-4-yl)-5-
(trifluoromethyl)tetrahydrofuran-2-carboxamide

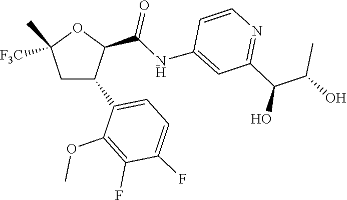 [SMILES: COc1c([C@@H]2C[C@](C)(C(F)(F)F)O[C@H]2C(=O)Nc2ccnc([C@H](O)[C@H](C)O)c2)ccc(F)c1F]

(2R,3S,5R)-3-(3,4-difluoro-2-methoxyphenyl)-N-
(2-((1S,2S)-1,2-dihydroxypropyl)pyridin-4-yl)-5-
methyl-5-(trifluoromethyl)tetrahydrofuran-2-
carboxamide

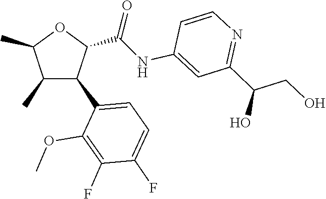 [SMILES: COc1c([C@H]2[C@@H](C)[C@@H](C)O[C@@H]2C(=O)Nc2ccnc([C@H](O)CO)c2)ccc(F)c1F]

(2S,3R,4R,5R)-3-(3,4-difluoro-2-
methoxyphenyl)-N-(2-((S)-1,2-
dihydroxyethyl)pyridin-4-yl)-4,5-
dimethyltetrahydrofuran-2-carboxamide

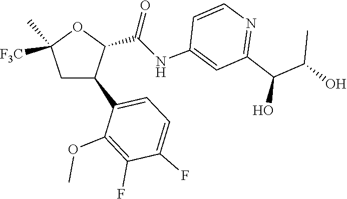 [SMILES: COc1c([C@H]2C[C@@](C)(C(F)(F)F)O[C@@H]2C(=O)Nc2ccnc([C@H](O)[C@H](C)O)c2)ccc(F)c1F]

(2S,3R,5S)-3-(3,4-difluoro-2-methoxyphenyl)-N-
(2-((1S,2S)-1,2-dihydroxypropyl)pyridin-4-yl)-5-
methyl-5-(trifluoromethyl)tetrahydrofuran-2-
carboxamide

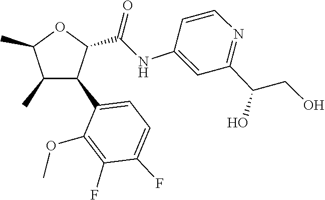 [SMILES: COc1c([C@H]2[C@@H](C)[C@@H](C)O[C@@H]2C(=O)Nc2ccnc([C@@H](O)CO)c2)ccc(F)c1F]

(2S,3R,4R,5R)-3-(3,4-difluoro-2-
methoxyphenyl)-N-(2-((R)-1,2-
dihydroxyethyl)pyridin-4-yl)-4,5-
dimethyltetrahydrofuran-2-carboxamide

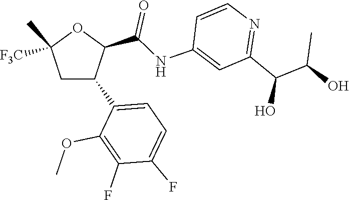 [SMILES: COc1c([C@@H]2C[C@](C)(C(F)(F)F)O[C@H]2C(=O)Nc2ccnc([C@H](O)[C@@H](C)O)c2)ccc(F)c1F]

(2R,3S,5R)-3-(3,4-difluoro-2-methoxyphenyl)-N-
(2-((1S,2R)-1,2-dihydroxypropyl)pyridin-4-yl)-5-
methyl-5-(trifluoromethyl)tetrahydrofuran-2-
carboxamide

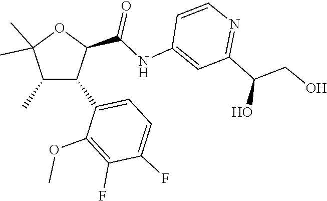 [SMILES: COc1c([C@H]2[C@H](C(=O)Nc3ccnc([C@H](O)CO)c3)OC(C)(C)[C@H]2C)ccc(F)c1F]

(2R,3S,4S)-3-(3,4-difluoro-2-methoxyphenyl)-N-
(2-((S)-1,2-dihydroxyethyl)pyridin-4-yl)-4,5,5-
trimethyltetrahydrofuran-2-carboxamide

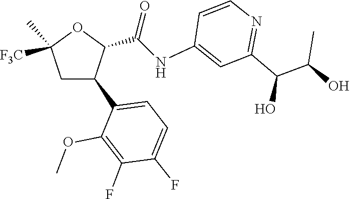 [SMILES: COc1c([C@H]2C[C@@](C)(C(F)(F)F)O[C@@H]2C(=O)Nc2ccnc([C@H](O)[C@@H](C)O)c2)ccc(F)c1F]

(2S,3R,5S)-3-(3,4-difluoro-2-methoxyphenyl)-N-
(2-((1S,2R)-1,2-dihydroxypropyl)pyridin-4-yl)-5-
methyl-5-(trifluoromethyl)tetrahydrofuran-2-
carboxamide

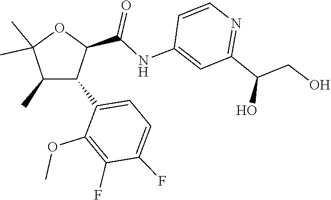 [SMILES: COc1c([C@@H]2[C@@H](C)C(C)(C)O[C@H]2C(=O)Nc2ccnc([C@H](O)CO)c2)ccc(F)c1F]

(2R,3S,4R)-3-(3,4-difluoro-2-methoxyphenyl)-N-
(2-((S)-1,2-dihydroxyethyl)pyridin-4-yl)-4,5,5-
trimethyltetrahydrofuran-2-carboxamide

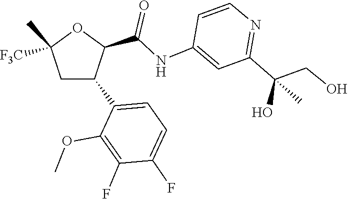 [SMILES: COc1c([C@@H]2C[C@](C)(C(F)(F)F)O[C@H]2C(=O)Nc2ccnc([C@](C)(O)CO)c2)ccc(F)c1F]

(2R,3S,5R)-3-(3,4-difluoro-2-methoxyphenyl)-N-
(2-((S)-1,2-dihydroxypropan-2-yl)pyridin-4-yl)-5-
methyl-5-(trifluoromethyl)tetrahydrofuran-2-
carboxamide

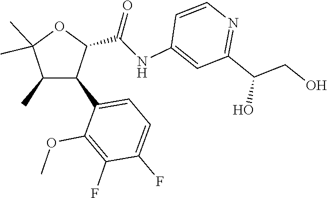 [SMILES: COc1c([C@@H]2[C@@H](C(=O)Nc3ccnc([C@@H](O)CO)c3)OC(C)(C)[C@@H]2C)ccc(F)c1F]

(2S,3R,4R)-3-(3,4-difluoro-2-methoxyphenyl)-N-
(2-((R)-1,2-dihydroxyethyl)pyridin-4-yl)-4,5,5-
trimethyltetrahydrofuran-2-carboxamide

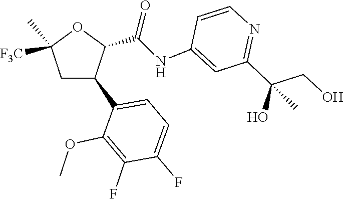 [SMILES: COc1c([C@H]2C[C@@](C)(C(F)(F)F)O[C@@H]2C(=O)Nc2ccnc([C@](C)(O)CO)c2)ccc(F)c1F]

(2S,3R,5S)-3-(3,4-difluoro-2-methoxyphenyl)-N-
(2-((S)-1,2-dihydroxypropan-2-yl)pyridin-4-yl)-5-
methyl-5-(trifluoromethyl)tetrahydrofuran-2-
carboxamide

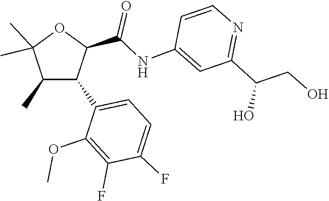 [SMILES: COc1c([C@@H]2[C@@H](C)C(C)(C)O[C@H]2C(=O)Nc2ccnc([C@@H](O)CO)c2)ccc(F)c1F]

(2R,3S,4R)-3-(3,4-difluoro-2-methoxyphenyl)-N-
(2-((R)-1,2-dihydroxyethyl)pyridin-4-yl)-4,5,5-
trimethyltetrahydrofuran-2-carboxamide

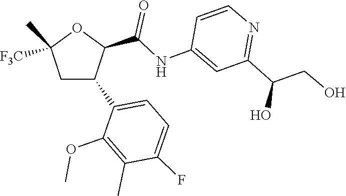 [SMILES: COc1c([C@@H]2C[C@](C)(C(F)(F)F)O[C@H]2C(=O)Nc2ccnc([C@H](O)CO)c2)ccc(F)c1C]

(2R,3S,5R)-N-(2-((S)-1,2-
dihydroxyethyl)pyridin-4-yl)-3-(4-fluoro-2-
methoxy-3-methylphenyl)-5-methyl-5-
(trifluoromethyl)tetrahydrofuran-2-carboxamide

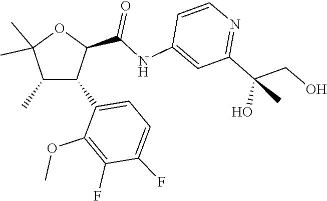 [SMILES: COc1c([C@H]2[C@H](C(=O)Nc3ccnc([C@@](C)(O)CO)c3)OC(C)(C)[C@H]2C)ccc(F)c1F]

(2R,3S,4S)-3-(3,4-difluoro-2-methoxyphenyl)-N-
(2-((R)-1,2-dihydroxypropan-2-yl)pyridin-4-yl)-
4,5,5-trimethyltetrahydrofuran-2-carboxamide

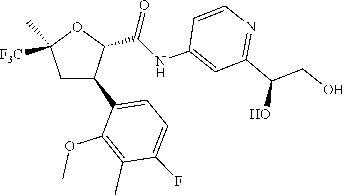 [SMILES: COc1c([C@H]2C[C@@](C)(C(F)(F)F)O[C@@H]2C(=O)Nc2ccnc([C@H](O)CO)c2)ccc(F)c1C]

(2S,3R,5S)-N-(2-((S)-1,2-dihydroxyethyl)pyridin-
4-yl)-3-(4-fluoro-2-methoxy-3-methylphenyl)-5-
methyl-5-(trifluoromethyl)tetrahydrofuran-2-
carboxamide

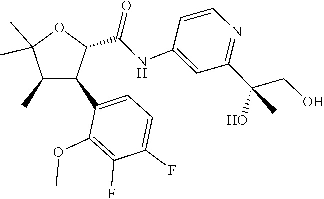 [SMILES: COc1c([C@@H]2[C@@H](C(=O)Nc3ccnc([C@@](C)(O)CO)c3)OC(C)(C)[C@@H]2C)ccc(F)c1F]

(2S,3R,4R)-3-(3,4-difluoro-2-methoxyphenyl)-N-
(2-((R)-1,2-dihydroxypropan-2-yl)pyridin-4-yl)-
4,5,5-trimethyltetrahydrofuran-2-carboxamide

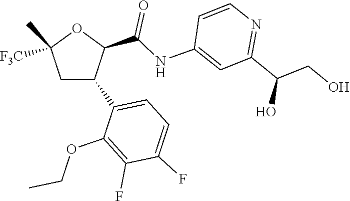 [SMILES: CCOc1c([C@@H]2C[C@](C)(C(F)(F)F)O[C@H]2C(=O)Nc2ccnc([C@H](O)CO)c2)ccc(F)c1F]

(2R,3S,5R)-N-(2-((S)-1,2-
dihydroxyethyl)pyridin-4-yl)-3-(2-ethoxy-3,4-
difluorophenyl)-5-methyl-5-
(trifluoromethyl)tetrahydrofuran-2-carboxamide

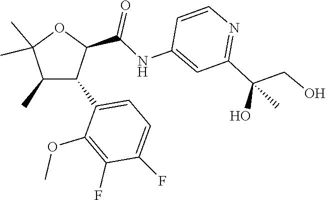 [SMILES: COc1c([C@@H]2[C@@H](C)C(C)(C)O[C@H]2C(=O)Nc2ccnc([C@](C)(O)CO)c2)ccc(F)c1F]

(2R,3S,4R)-3-(3,4-difluoro-2-methoxyphenyl)-N-
(2-((S)-1,2-dihydroxypropan-2-yl)pyridin-4-yl)-
4,5,5-trimethyltetrahydrofuran-2-carboxamide

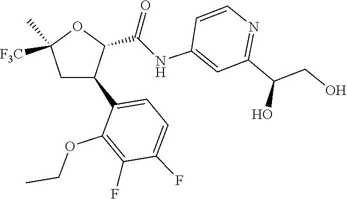 [SMILES: CCOc1c([C@H]2C[C@@](C)(C(F)(F)F)O[C@@H]2C(=O)Nc2ccnc([C@H](O)CO)c2)ccc(F)c1F]

(2S,3R,5S)-N-(2-((S)-1,2-dihydroxyethyl)pyridin-
4-yl)-3-(2-ethoxy-3,4-difluorophenyl)-5-methyl-5-
(trifluoromethyl)tetrahydrofuran-2-carboxamide

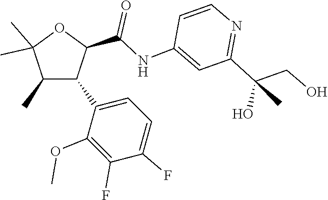 [SMILES: COc1c([C@@H]2[C@@H](C)C(C)(C)O[C@H]2C(=O)Nc2ccnc([C@@](C)(O)CO)c2)ccc(F)c1F]

(2R,3S,4R)-3-(3,4-difluoro-2-methoxyphenyl)-N-
(2-((R)-1,2-dihydroxypropan-2-yl)pyridin-4-yl)-
4,5,5-trimethyltetrahydrofuran-2-carboxamide

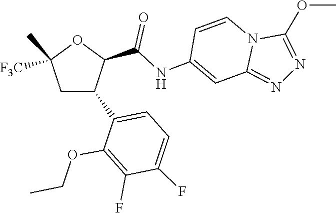 [SMILES: CCOc1c([C@@H]2C[C@](C)(C(F)(F)F)O[C@H]2C(=O)Nc2ccn3c(OC)nnc3c2)ccc(F)c1F]

(2R,3S,5R)-3-(2-ethoxy-3,4-difluorophenyl)-N-(3-
methoxy-[1,2,4]triazolo[4,3-a]pyridin-7-yl)-5-
methyl-5-(trifluoromethyl)tetrahydrofuran-2-
carboxamide

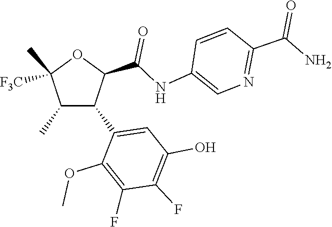 [SMILES: COc1c([C@H]2[C@H](C(=O)Nc3ccc(C(N)=O)nc3)O[C@@](C)(C(F)(F)F)[C@H]2C)cc(O)c(F)c1F]

5-((2R,3S,4S,5R)-3-(3,4-difluoro-5-hydroxy-2-
methoxyphenyl)-4,5-dimethyl-5-
(trifluoromethyl)tetrahydrofuran-2-
carboxamido)picolinamide

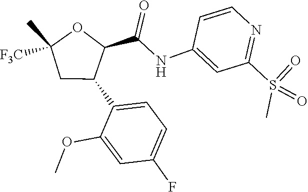 [SMILES: COc1cc(F)ccc1[C@@H]1C[C@](C)(C(F)(F)F)O[C@H]1C(=O)Nc1ccnc(S(C)(=O)=O)c1]

(2R,3S,5R)-3-(4-fluoro-2-methoxyphenyl)-5-
methyl-N-(2-(methylsulfonyl)pyridin-4-yl)-5-
(trifluoromethyl)tetrahydrofuran-2-carboxamide

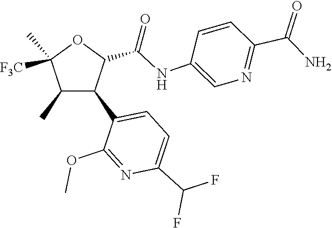

5-((2S,3R,4R,5S)-3-(6-(difluoromethyl)-2-
methoxypyridin-3-yl)-4,5-dimethyl-5-
(trifluoromethyl)tetrahydrofuran-2-
carboxamido)picolinamide

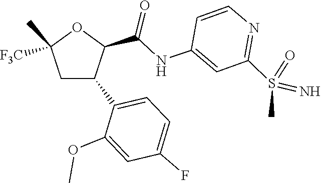 [SMILES: COc1cc(F)ccc1[C@@H]1C[C@](C)(C(F)(F)F)O[C@H]1C(=O)Nc1ccnc([S@@](C)(=N)=O)c1]

(2R,3S,5R)-3-(4-fluoro-2-methoxyphenyl)-5-
methyl-N-(2-(S-methylsulfonimidoyl)pyridin-4-
yl)-5-(trifluoromethyl)tetrahydrofuran-2-
carboxamide

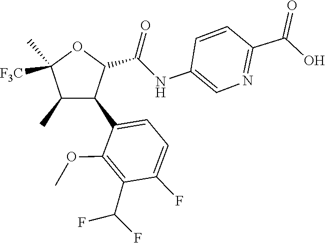

5-((2S,3R,4R,5S)-3-(3-(difluoromethyl)-4-fluoro-
2-methoxyphenyl)-4,5-dimethyl-5-
(trifluoromethyl)tetrahydrofuran-2-
carboxamido)picolinic acid

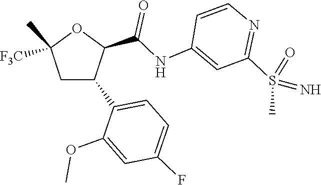 [SMILES: COc1cc(F)ccc1[C@@H]1C[C@](C)(C(F)(F)F)O[C@H]1C(=O)Nc1ccnc([S@](C)(=N)=O)c1]

(2R,3S,5R)-3-(4-fluoro-2-methoxyphenyl)-5-
methyl-N-(2-(S-methylsulfonimidoyl)pyridin-4-
yl)-5-(trifluoromethyl)tetrahydrofuran-2-
carboxamide

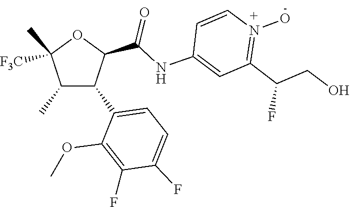

4-((2R,3S,4S,5R)-3-(3,4-difluoro-2-
methoxyphenyl)-4,5-dimethyl-5-
(trifluoromethyl)tetrahydrofuran-2-
carboxamido)-2-((R)-1-fluoro-2-
hydroxyethyl)pyridine 1-oxide

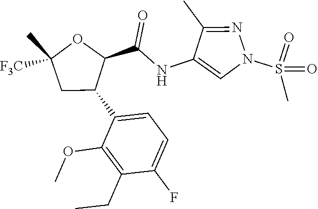

(2R,3S,5R)-3-(3-ethyl-4-fluoro-2-
methoxyphenyl)-5-methyl-N-(3-methyl-1-
(methylsulfonyl)-1H-pyrazol-4-yl)-5-
(trifluoromethyl)tetrahydrofuran-2-carboxamide

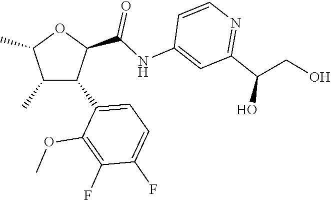 [SMILES: COc1c([C@@H]2[C@H](C)[C@H](C)O[C@H]2C(=O)Nc2ccnc([C@H](O)CO)c2)ccc(F)c1F]

(2R,3S,4S,5S)-3-(3,4-difluoro-2-
methoxyphenyl)-N-(2-((S)-1,2-
dihydroxyethyl)pyridin-4-yl)-4,5-
dimethyltetrahydrofuran-2-carboxamide

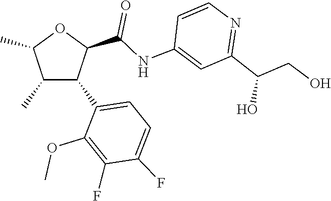 [SMILES: COc1c([C@@H]2[C@H](C)[C@H](C)O[C@H]2C(=O)Nc2ccnc([C@@H](O)CO)c2)ccc(F)c1F]

(2R,3S,4S,5S)-3-(3,4-difluoro-2-
methoxyphenyl)-N-(2-((R)-1,2-
dihydroxyethyl)pyridin-4-yl)-4,5-
dimethyltetrahydrofuran-2-carboxamide

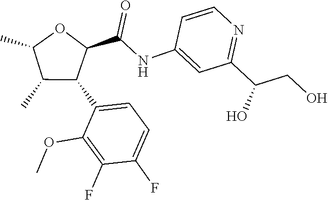 [SMILES: COc1c([C@@H]2[C@H](C)[C@H](C)O[C@H]2C(=O)Nc2ccnc([C@@H](O)CO)c2)ccc(F)c1F]

(2R,3S,4S,5S)-3-(3,4-difluoro-2-
methoxyphenyl)-N-(2-((R)-1,2-
dihydroxyethyl)pyridin-4-yl)-4,5-
dimethyltetrahydrofuran-2-carboxamide

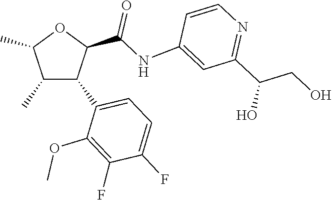 [SMILES: COc1c([C@@H]2[C@H](C)[C@H](C)O[C@H]2C(=O)Nc2ccnc([C@@H](O)CO)c2)ccc(F)c1F]

(2R,3S,4S,5S)-3-(3,4-difluoro-2-
methoxyphenyl)-N-(2-((R)-1,2-
dihydroxyethyl)pyridin-4-yl)-4,5-
dimethyltetrahydrofuran-2-carboxamide

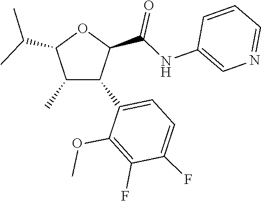 [SMILES: COc1c([C@@H]2[C@H](C)[C@H](C(C)C)O[C@H]2C(=O)Nc2cccnc2)ccc(F)c1F]

(2R,3S,4S,5S)-3-(3,4-difluoro-2-
methoxyphenyl)-5-isopropyl-4-methyl-N-
(pyridin-3-yl)tetrahydrofuran-2-carboxamide

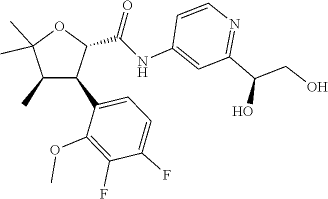 [SMILES: COc1c([C@@H]2[C@@H](C(=O)Nc3ccnc([C@H](O)CO)c3)OC(C)(C)[C@@H]2C)ccc(F)c1F]

(2S,3R,4R)-3-(3,4-difluoro-2-methoxyphenyl)-N-
(2-((S)-1,2-dihydroxyethyl)pyridin-4-yl)-4,5,5-
trimethyltetrahydrofuran-2-carboxamide

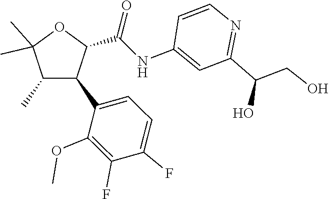 [SMILES: COc1c([C@@H]2[C@@H](C(=O)Nc3ccnc([C@H](O)CO)c3)OC(C)(C)[C@H]2C)ccc(F)c1F]

(2S,3R,4S)-3-(3,4-difluoro-2-methoxyphenyl)-N-
(2-((S)-1,2-dihydroxyethyl)pyridin-4-yl)-4,5,5-
trimethyltetrahydrofuran-2-carboxamide

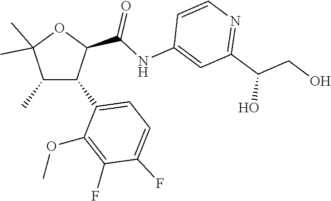 [SMILES: COc1c([C@H]2[C@H](C(=O)Nc3ccnc([C@@H](O)CO)c3)OC(C)(C)[C@H]2C)ccc(F)c1F]

(2R,3S,4S)-3-(3,4-difluoro-2-methoxyphenyl)-N-
(2-((R)-1,2-dihydroxyethyl)pyridin-4-yl)-4,5,5-
trimethyltetrahydrofuran-2-carboxamide

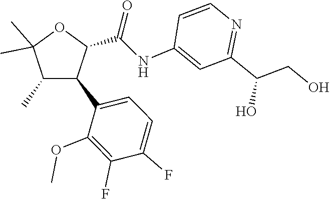 [SMILES: COc1c([C@@H]2[C@@H](C(=O)Nc3ccnc([C@@H](O)CO)c3)OC(C)(C)[C@H]2C)ccc(F)c1F]

(2S,3R,4S)-3-(3,4-difluoro-2-methoxyphenyl)-N-
(2-((R)-1,2-dihydroxyethyl)pyridin-4-yl)-4,5,5-
trimethyltetrahydrofuran-2-carboxamide

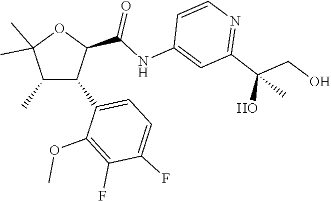 [SMILES: COc1c([C@H]2[C@H](C(=O)Nc3ccnc([C@](C)(O)CO)c3)OC(C)(C)[C@H]2C)ccc(F)c1F]

(2R,3S,4S)-3-(3,4-difluoro-2-methoxyphenyl)-N-
(2-((S)-1,2-dihydroxypropan-2-yl)pyridin-4-yl)-
4,5,5-trimethyltetrahydrofuran-2-carboxamide

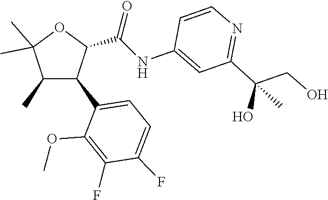 [SMILES: COc1c([C@@H]2[C@@H](C(=O)Nc3ccnc([C@](C)(O)CO)c3)OC(C)(C)[C@@H]2C)ccc(F)c1F]

(2S,3R,4R)-3-(3,4-difluoro-2-methoxyphenyl)-N-
(2-((S)-1,2-dihydroxypropan-2-yl)pyridin-4-yl)-
4,5,5-trimethyltetrahydrofuran-2-carboxamide

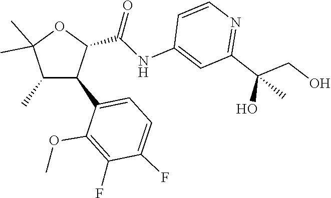 [SMILES: COc1c([C@@H]2[C@@H](C(=O)Nc3ccnc([C@](C)(O)CO)c3)OC(C)(C)[C@H]2C)ccc(F)c1F]

(2S,3R,4S)-3-(3,4-difluoro-2-methoxyphenyl)-N-
(2-((S)-1,2-dihydroxypropan-2-yl)pyridin-4-yl)-
4,5,5-trimethyltetrahydrofuran-2-carboxamide

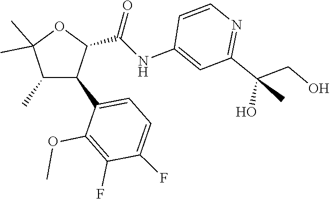 [SMILES: COc1c([C@@H]2[C@@H](C(=O)Nc3ccnc([C@@](C)(O)CO)c3)OC(C)(C)[C@H]2C)ccc(F)c1F]

(2S,3R,4S)-3-(3,4-difluoro-2-methoxyphenyl)-N-
(2-((R)-1,2-dihydroxypropan-2-yl)pyridin-4-yl)-
4,5,5-trimethyltetrahydrofuran-2-carboxamide

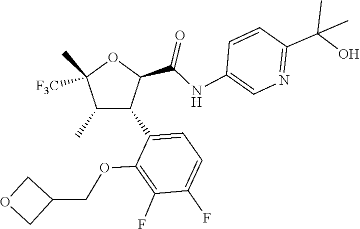 [SMILES: C[C@H]1[C@@H](c2ccc(F)c(F)c2OCC2COC2)[C@H](C(=O)Nc2ccc(C(C)(C)O)nc2)O[C@@]1(C)C(F)(F)F]

(2R,3S,4S,5R)-3-(3,4-difluoro-2-(oxetan-3-
ylmethoxy)phenyl)-N-(6-(2-hydroxypropan-2-
yl)pyridin-3-yl)-4,5-dimethyl-5-
(trifluoromethyl)tetrahydrofuran-2-carboxamide

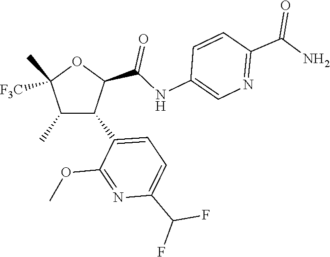 [SMILES: COc1nc(C(F)F)ccc1[C@H]1[C@H](C(=O)Nc2ccc(C(N)=O)nc2)O[C@@](C)(C(F)(F)F)[C@H]1C]

5-((2R,3S,4S,5R)-3-(6-(difluoromethyl)-2-
methoxypyridin-3-yl)-4,5-dimethyl-5-
(trifluoromethyl)tetrahydrofuran-2-
carboxamido)picolinamide

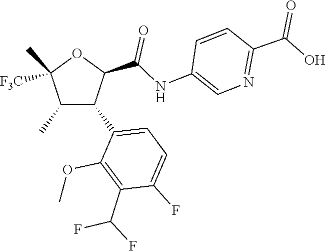 [SMILES: COc1c([C@H]2[C@H](C(=O)Nc3ccc(C(=O)O)nc3)O[C@@](C)(C(F)(F)F)[C@H]2C)ccc(F)c1C(F)F]

5-((2R,3S,4S,5R)-3-(3-(difluoromethyl)-4-fluoro-
2-methoxyphenyl)-4,5-dimethyl-5-
(trifluoromethyl)tetrahydrofuran-2-
carboxamido)picolinic acid

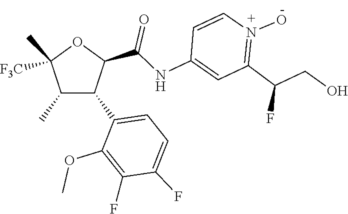

4-((2R,3S,4S,5R)-3-(3,4-difluoro-2-
methoxyphenyl)-4,5-dimethyl-5-
(trifluoromethyl)tetrahydrofuran-2-
carboxamido)-2-((S)-1-fluoro-2-
hydroxyethyl)pyridine 1-oxide

In some embodiments, the invention relates to a compound of formula

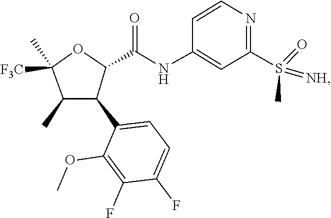 [SMILES: COc1c([C@@H]2[C@@H](C(=O)Nc3ccnc([S@@](C)(=N)=O)c3)O[C@](C)(C(F)(F)F)[C@@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

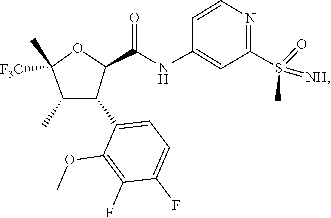 [SMILES: COc1c([C@H]2[C@H](C(=O)Nc3ccnc([S@@](C)(=N)=O)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

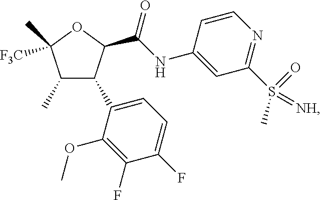 [SMILES: COc1c([C@H]2[C@H](C(=O)Nc3ccnc([S@](C)(=N)=O)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

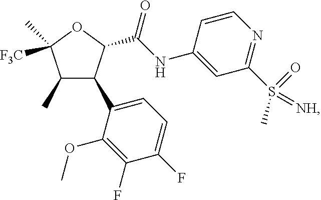 [SMILES: COc1c([C@@H]2[C@@H](C(=O)Nc3ccnc([S@](C)(=N)=O)c3)O[C@](C)(C(F)(F)F)[C@@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

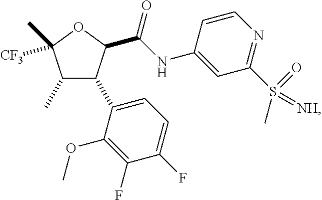 [SMILES: COc1c([C@H]2[C@H](C(=O)Nc3ccnc(S(C)(=N)=O)c3)O[C@@](C)(C(F)(F)F)[C@H]2C)ccc(F)c1F]

or a pharmaceutically acceptable salt thereof, wherein the compound has the absolute and relative stereochemistry of the second eluting isomer when the four stereoisomers of the foregoing formula are separated by SFC as described in Example 1. In other embodiments, the invention relates to the foregoing compound in non-salt form. Such compound is considered to be a “compound of the invention,” as that term is used herein.

In some embodiments, the invention relates to a compound of formula

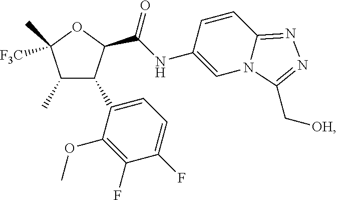 [SMILES: COc1c([C@H]2[C@H](C(=O)Nc3ccc4nnc(CO)n4c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

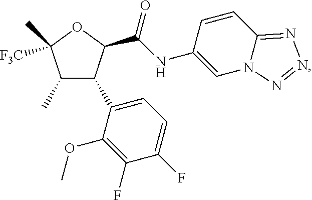 [SMILES: COc1c([C@H]2[C@H](C(=O)Nc3ccc4nnnn4c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

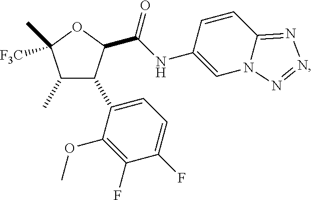 [SMILES: COc1c([C@H]2[C@H](C(=O)Nc3ccc4nnnn4c3)O[C@@](C)(C(F)(F)F)[C@H]2C)ccc(F)c1F]

or a pharmaceutically acceptable salt thereof, wherein the compound has the absolute stereochemistry of the second eluting isomer when rac-(2R,3S,4S,5R)-3-(3,4-difluoro-2-methoxyphenyl)-4,5-dimethyl-N-(tetrazolo[1,5-a]pyridin-6-yl)-5-(trifluoromethyl)tetrahydrofuran-2-carboxamideis separated by SFC as described in Example 10. In other embodiments, the invention relates to the foregoing compound in non-salt form. Such compound is considered to be a “compound of the invention,” as that term is used herein.

In some embodiments, the invention relates to a compound of formula

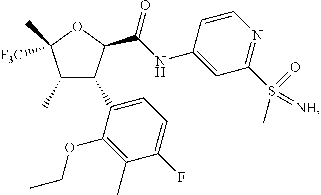 [SMILES: CCOc1c([C@H]2[C@H](C(=O)Nc3ccnc(S(C)(=N)=O)c3)O[C@@](C)(C(F)(F)F)[C@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

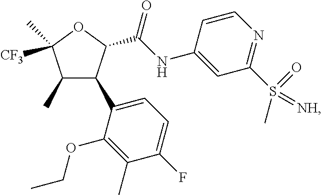 [SMILES: CCOc1c([C@@H]2[C@@H](C(=O)Nc3ccnc(S(C)(=N)=O)c3)O[C@](C)(C(F)(F)F)[C@@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

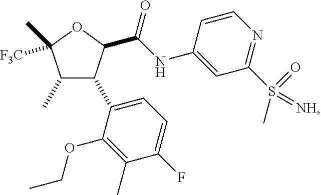 [SMILES: CCOc1c([C@H]2[C@H](C(=O)Nc3ccnc(S(C)(=N)=O)c3)O[C@@](C)(C(F)(F)F)[C@H]2C)ccc(F)c1C]

or a pharmaceutically acceptable salt thereof, wherein the compound has the absolute stereochemistry of the second eluting isomers when the four stereoisomers of the foregoing formula are separated by SFC as described in Example 4. In other embodiments, the invention relates to the foregoing compound in non-salt form. Such compound is considered to be a “compound of the invention,” as that term is used herein.

In some embodiments, the invention relates to a compound of formula

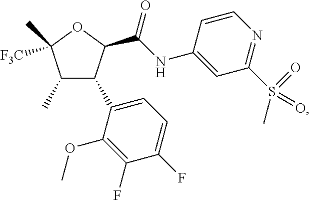 [SMILES: COc1c([C@H]2[C@H](C(=O)Nc3ccnc(S(C)(=O)=O)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

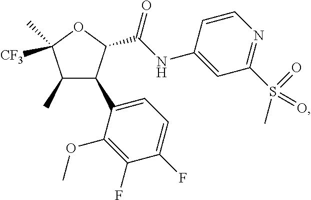 [SMILES: COc1c([C@@H]2[C@@H](C(=O)Nc3ccnc(S(C)(=O)=O)c3)O[C@](C)(C(F)(F)F)[C@@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

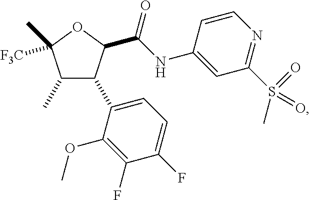 [SMILES: COc1c([C@H]2[C@H](C(=O)Nc3ccnc(S(C)(=O)=O)c3)O[C@@](C)(C(F)(F)F)[C@H]2C)ccc(F)c1F]

or a pharmaceutically acceptable salt thereof, wherein the compound has the absolute stereochemistry of the second eluting isomer when rac-(2R,3S,4S,5R)-3-(3,4-difluoro-2-methoxyphenyl)-4,5-dimethyl-N-(2-(methylsulfonyl)pyridin-4-yl)-5-(trifluoromethyl)tetrahydrofuran-2-carboxamide is separated by SFC as described in Example 1, Step 12. In other embodiments, the invention relates to the foregoing compound in non-salt form. Such compound is considered to be a “compound of the invention,” as that term is used herein.

In some embodiments, the invention relates to a compound of formula

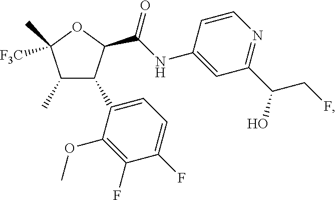 [SMILES: COc1c([C@H]2[C@H](C(=O)Nc3ccnc([C@@H](O)CF)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

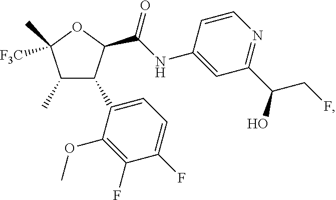 [SMILES: COc1c([C@H]2[C@H](C(=O)Nc3ccnc([C@H](O)CF)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

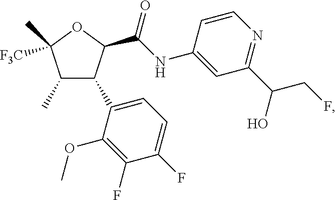 [SMILES: COc1c([C@H]2[C@H](C(=O)Nc3ccnc(C(O)CF)c3)O[C@@](C)(C(F)(F)F)[C@H]2C)ccc(F)c1F]

or a pharmaceutically acceptable salt thereof, wherein the compound has the absolute stereochemistry corresponding to the second eluting isomer when the two stereoisomers of (2R,3S,4S,5R)—N-(2-(1-((tert-butyldimethylsilyl)oxy)-2-fluoroethyl)pyridin-4-yl)-3-(3,4-difluoro-2-methoxyphenyl)-4,5-dimethyl-5-(trifluoromethyl)tetrahydrofuran-2-carboxamide are separated by SFC as described for Example 7. In other embodiments, the invention relates to the foregoing compound in non-salt form. Such compound is considered to be a “compound of the invention,” as that term is used herein.

In some embodiments, the invention relates to a compound of formula

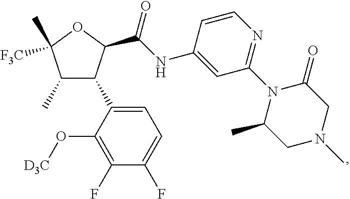 [SMILES: [2H]C([2H])([2H])Oc1c([C@H]2[C@H](C(=O)Nc3ccnc(N4C(=O)CN(C)C[C@H]4C)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

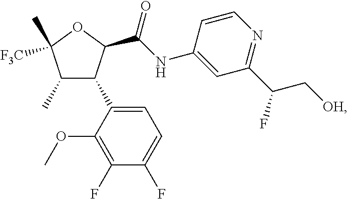 [SMILES: COc1c([C@H]2[C@H](C(=O)Nc3ccnc([C@@H](F)CO)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

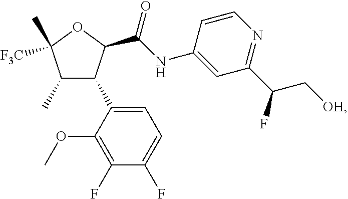 [SMILES: COc1c([C@H]2[C@H](C(=O)Nc3ccnc([C@H](F)CO)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

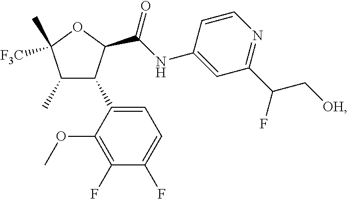 [SMILES: COc1c([C@H]2[C@H](C(=O)Nc3ccnc(C(F)CO)c3)O[C@@](C)(C(F)(F)F)[C@H]2C)ccc(F)c1F]

or a pharmaceutically acceptable salt thereof, wherein the compound has the absolute stereochemistry corresponding to the second eluting isomer when the two stereoisomers of (2R,3S,4S,5R)—N-(2-(2-(tert-butoxy)-1-fluoroethyl)pyridin-4-yl)-3-(3,4-difluoro-2-methoxyphenyl)-4,5-dimethyl-5-(trifluoromethyl)tetrahydrofuran-2-carboxamide are separated by SFC as described for Example 10. In other embodiments, the invention relates to the foregoing compound in non-salt form. Such compound is considered to be a “compound of the invention,” as that term is used herein.

In some embodiments, the invention relates to a compound of formula

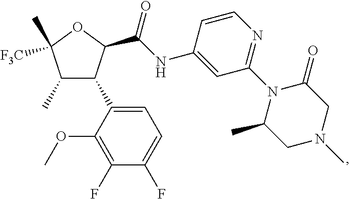 [SMILES: COc1c([C@H]2[C@H](C(=O)Nc3ccnc(N4C(=O)CN(C)C[C@H]4C)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

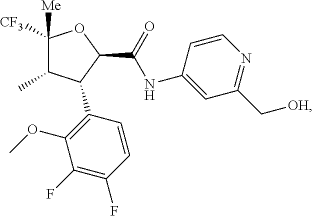 [SMILES: COc1c([C@H]2[C@H](C(=O)Nc3ccnc(CO)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

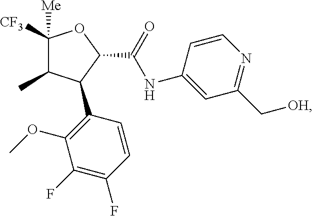 [SMILES: COc1c([C@@H]2[C@@H](C(=O)Nc3ccnc(CO)c3)O[C@](C)(C(F)(F)F)[C@@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

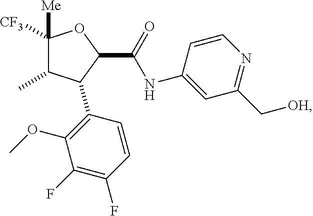 [SMILES: COc1c([C@H]2[C@H](C(=O)Nc3ccnc(CO)c3)O[C@@](C)(C(F)(F)F)[C@H]2C)ccc(F)c1F]

or a pharmaceutically acceptable salt thereof, wherein the compound has the absolute stereochemistry corresponding to the second eluting isomer when the two enantiomers of rac-(2R,3S,4S,5R)—N-(2-(((tert-butyldimethylsilyl)oxy)methyl)pyridin-4-yl)-3-(3,4-difluoro-2-methoxyphenyl)-4,5-dimethyl-5-(trifluoromethyl)tetrahydrofuran-2-carboxamide are separated by SFC as described in Example 1. In other embodiments, the invention relates to the foregoing compound in non-salt form. Such compound is considered to be a “compound of the invention,” as that term is used herein.

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose, starches such as corn starch and potato starch, cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols, such a propylene glycol or polyethylene glycol, esters such as ethyl oleate and ethyl laurate, agar, buffering agents such as magnesium hydroxide and aluminum hydroxide, alginic acid, pyrogen-free water, isotonic saline, Ringer's solution, ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/hemiation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of herniorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/hemiation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are optionally formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder, the activity of the specific compound or salt employed, the specific composition employed, the age, body weight, general health, sex and diet of the subject, the time of administration, route of administration, and rate of excretion of the specific compound or salt employed, the duration of the treatment, drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin, naphthylalkanones such as Nabumetone, oxicams such as Piroxicam, para-aminophenol derivatives, such as Acetaminophen, propionic acids such as Fenoprofen, Flurbiprofen, Ibuprofen, Ketoprofen, Naproxen, Naproxen sodium, Oxaprozin, salicylates such as Aspirin, Choline magnesium trisalicylate, Diflunisal, fenamates such as meclofenamic acid, Mefenamic acid, and pyrazoles such as Phenylbutazone), or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile, 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504),
(37) a sodium channel blocker, such as lidocaine, lidocaine plus tetracaine cream (ZRS-201) or eslicarbazepine acetate;
(38) a Na_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607), WO2012/106499 (US2012196869), WO2012/112743 (US2012245136), WO2012/125613 (US2012264749), WO2012/116440 (US2014187533), WO2011026240 (US2012220605), U.S. Pat. Nos. 8,883,840, 8,466,188, WO2013/109521 (US2015005304), WO2020/117626, and CN111217776,the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/261114, WO2020/140959, WO2020/151728, WO2021/032074, CN112390745, CN111808019, CN112225695, CN112457294, CN112300051, CN112300069, CN112441969, and CN112479996 (WO2021/047622), the entire contents of each application hereby incorporated by reference;
(39a) a Na_{V}1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, 4-(2-fluoro-6-(2-methoxy-4-(trifluoromethoxy)phenoxy)-3-(trifluoromethyl)benzamido)picolinamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof,
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide, N-(6-amino-5-(2-chloro-5-methoxyphenyl)pyridin-2-yl)-1-methyl-1H-pyrazole-5-carboxamide, or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NE06860, FTABS, CNTX4975, MCP101, MDR16523, or MDR652, a EGRI inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NEO1940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427 or ST-2578 and those disclosed in WO2010129864, WO2015157559, WO2017059385, WO2018183781, WO2018183782, WO2020072835, and WO2022036297 the entire contents of each application hereby incorporated by reference. In some embodiments, the additional therapeutic agent is a Na_{V}1.7 blocker disclosed in WO2020072835. In some embodiments, the additional therapeutic agent is a Na_{V}1.7 blocker disclosed in WO2022036297.

In another embodiment, the additional therapeutic agent is ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, or XT-150.

In another embodiment, the additional therapeutic agent is Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623, ETX-801, or ACD440.

In another embodiment, the additional therapeutic agent is a compound disclosed in WO2021257490, WO2021257420, WO2021257418, WO2020014246, WO2020092187, WO2020092667, WO2020261114, CN112457294, CN112225695, CN111808019, WO2021032074, WO2020151728, WO2020140959, WO2022037641, WO2022037647, CN112300051, CN112300069, WO2014120808, WO2015089361, WO2019014352, WO2021113627, WO2013086229, WO2013134518, WO2014211173, WO2014201206, WO2016141035, WO2021252818, WO2021252822, and WO2021252820.

In some embodiments, the additional therapeutic agent is a compound disclosed in WO2013086229. In some embodiments, the additional therapeutic agent is a compound disclosed in WO2013134518. In some embodiments, the additional therapeutic agent is a compound disclosed in WO2014211173. In some embodiments, the additional therapeutic agent is a compound disclosed in WO2014201206. In some embodiments, the additional therapeutic agent is a compound disclosed in WO2016141035. In some embodiments, the additional therapeutic agent is a compound disclosed in WO2021252818. In some embodiments, the additional therapeutic agent is a compound disclosed in WO2021252822. In some embodiments, the additional therapeutic agent is a compound disclosed in WO2021252820. In some embodiments, the additional therapeutic agent is a compound disclosed in WO2020072835. In some embodiments, the additional therapeutic agent is a compound disclosed in WO2022036297.

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562, 5,886,026, and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena, and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### Enumerated Embodiments

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.

1. A compound of formula (I)

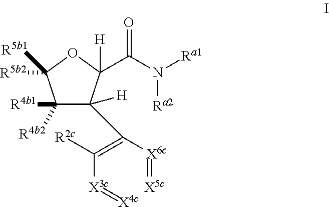

or a pharmaceutically acceptable salt thereof, wherein:

R^{a1} is —(C(R^{a′})₂)ₚ—R^{a″},

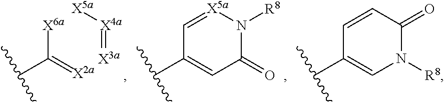

5-membered heteroaryl, 3-7 membered heterocycloalkyl, 9-10 membered aryl, or 9-10 membered heteroaryl, wherein said 5-membered heteroaryl, 3-7 membered heterocycloalkyl, 9-10 membered aryl, or 9-10 membered heteroaryl is optionally substituted by one or more R^{a3};
R^{a2} is H;
or R^{a1} and R^{a2} together with the nitrogen to which they are attached form a 3-10 membered heterocycloalkyl, wherein said 3-10 membered heterocycloalkyl is optionally substituted by one or more R^{a3};
each R^{a′} is independently H or methyl optionally substituted by OH, or two R^{a′} together with the atom or atoms to which they are attached form C₃-C₆ cycloalkyl, 3-7 membered heterocycloalkyl, or oxo; R^{a″} is C₃-C₆ cycloalkyl, 3-7 membered heterocycloalkyl, 5-10 membered heteroaryl, phenyl, —NR⁹R¹⁰, —OR¹¹, or —CN, wherein said 5-10 membered heteroaryl, 3-7 membered heterocycloalkyl, or phenyl is optionally substituted by one or more R¹³;
each R^{a3} is independently halo, C₁-C₆ alkyl, C₁-C₆ haloalkyl, 3-7 membered heterocycloalkyl, —C(O)C₁-C₆ alkyl, —OR¹¹, —C(O)NR⁹R¹⁰, or —S(O)₂R⁷, wherein said C₁-C₆ alkyl, C₁-C₆ haloalkyl, 3-7 membered heterocycloalkyl or —C(O)C₁-C₆ alkyl is optionally substituted by one or more halo, —OR¹¹, —CN, or —NR⁹R¹⁰, or two R^{a3} attached to the same atom combine to form oxo, or two R^{a3} attached to adjacent atoms together with the atoms to which they are attached combine to form a fused 3-7 membered ring containing up to two heteroatoms selected from the group consisting of N, O, and S;
X^{2a} is N, N⁺—O—, or C—R^{2a};
X^{3a} is N, N⁺—O—, or C—R^{3a};
X^{4a} is N, N⁺—O—, or C—R^{4a};
X^{5a} is N, N⁺—O—, C—R^{5a}, or N⁺—(C₁-C₆ alkyl)Y⁻, wherein Y⁻ is a monovalent anion;
X^{6a} is N, N⁺—O—, or C—R^{6a};
R^{2a} is H, halo, C₁-C₆ alkyl, or C₁-C₆ haloalkyl;
R^{3a} is H, halo, C₁-C₆ alkyl, C₁-C₆ haloalkyl, C₁-C₆ alkoxy, 3-9 membered heterocycloalkyl, 5-membered heteroaryl, —CN, —OR¹¹, —COOH, —NR⁹C(O)C₁-C₆ alkyl, —S(O)₂R⁷, —S(O)(NR⁹)R⁷, —S(O)NR⁹R¹⁰, —S(O)R⁷, or —P(O)(C₁-C₆ alkyl)₂, wherein said C₁-C₆ alkyl, C₁-C₆ alkoxy, 3-9 membered heterocycloalkyl, 5-membered heteroaryl, or —NR⁹C(O)C₁-C₆ alkyl is optionally substituted by one or more R¹², C₃-C₆ cycloalkyl, —NR⁹R¹⁰, —OR¹¹, —CN, or 3-7 membered heterocycloalkyl optionally substituted by one or more R²;
R^{4a} is H, halo, C₁-C₆ alkyl, C₁-C₆ haloalkyl, C₂-C₆ alkynyl, C₁-C₆ alkoxy, 3-7 membered heterocycloalkyl, 5-6 membered heteroaryl, —CN, —C(O)NR⁹R¹⁰, —C(O)OH, —OR¹¹, —NR⁹R¹⁰, —NR⁹C(O)C₁-C₆ alkyl, —S—C₁-C₆ alkyl, —S(O)(NR⁹)R⁷, —S(O)NR⁹R¹⁰, or —P(O)(C₁-C₆ alkyl)₂, wherein said C₁-C₆ alkyl, C₁-C₆ alkoxy, 3-7 membered heterocycloalkyl, 5-6 membered heteroaryl, or C₂-C₆ alkynyl is optionally substituted by one or more halo, —OR¹¹, 3-7 membered heterocycloalkyl, —NR⁹R¹⁰, C₁-C₆ alkyl, or —S(O)₂R⁷; R^{5a} is H, halo, C₁-C₆ alkyl, C₁-C₆ haloalkyl, or —S(O)₂R⁷; R^{6a} is H, halo, C₁-C₆ alkyl, or C₁-C₆ haloalkyl;
or R^{3a} and R⁴ together with the atoms to which they are attached form a ring of formula:

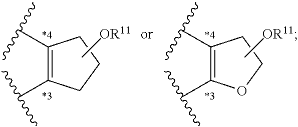

R⁷ is C₁-C₆ alkyl or 3-7 membered heterocycloalkyl, wherein said C₁-C₆ alkyl or 3-7 membered heterocycloalkyl is optionally substituted by one or more —OR¹¹ or C₁-C₆ alkyl;
R⁸ is H or C₁-C₆ alkyl;
R⁹ and R¹⁰ are each independently H, C₁-C₆ alkyl, 3-7 membered heterocycloalkyl, C₃-C₆ cycloalkyl, —OH, —CN, or —S(O)₂R⁷, wherein said C₁-C₆ alkyl is optionally substituted by one or more —OR¹¹, or R⁹ and R¹⁰ together with the atom to which they are attached form a 37 membered heterocycloalkyl;
each R¹¹ is independently H, C₁-C₆ alkyl, C₁-C₆ haloalkyl, a 3-7 membered heterocycloalkyl optionally substituted with —OH, or a 3-7 membered cycloalkyl optionally substituted with —OH; each R¹² is independently halo, C₁-C₆ alkyl, or —OR¹¹, or two R¹² together with the atom they are attached combine to form oxo;
each R¹³ is independently halo, C₁-C₆ alkyl, or —CONH₂, wherein said C₁-C₆ alkyl is optionally substituted by one or more —OR¹¹, or two R¹³ together with the atom they are attached combine to form oxo;
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, or C₁-C₆ haloalkyl;
X^{3c} is N or C—R^{3c};
X^{4c} is N or C—R^{4c};
X^{5c} is N or C—R^{5c};
X^{6c} is N or C—R^{6c};
R^{2c} is H, —OH, halo, C₁-C₆ alkyl, C₂-C₆ alkenyl, C₁-C₆ haloalkyl, C₁-C₆ alkoxy, C₁-C₆ haloalkoxy, -L¹-(C₁-C₆ alkylene)-OR⁵, -L¹-(C₁-C₆ alkenylene)-OR¹⁵, -L¹-(C₁-C₆ alkylene)-NR¹⁶R¹⁷, -L¹-(C₁-C₆ alkylene)-N═S(O)(C₁-C₃ alkyl)₂, or L1-L2-R¹⁴;
R¹⁴ is C₃-C₆ cycloalkyl, 3-8 membered heterocycloalkyl, 5- or 6-membered heteroaryl, —C(O)O(C₁-C₆ alkyl), —COOH, or —C(O)NR¹⁶R¹⁷, wherein said C₃-C₆ cycloalkyl, 3-8 membered heterocycloalkyl or 5- or 6-membered heteroaryl is optionally substituted by one or more halo, —OH, C₁-C₆ alkyl, C₁-C₆ haloalkyl, C₁-C₆ alkoxy, or C₁-C₆ haloalkoxy;
R¹⁵ is H, C₁-C₆ alkyl, or C₁-C₆ haloalkyl:
R¹⁶ and R¹⁷ are each independently H, —OH, C₁-C₆ alkyl, or 3-7 membered heterocycloalkyl;
R^{3c} is H, halo, C₁-C₆ alkyl, C₁-C₆ haloalkyl, or —(C₁-C₆ alkylene)-(C₁-C₆ alkoxy);
R^{4c} is H, halo, C₁-C₆ alkyl, or C₁-C₆ haloalkyl;
R^{5c} is H, halo, C₁-C₆ alkyl, or C₁-C₆ haloalkyl; and
R^{6c} is H, halo, C₁-C₆ alkyl, C₁-C₆ haloalkyl, or C₁-C₆ alkoxy;
L¹ is a bond or O;
L² is a bond or C₁-C₆ alkylene; and
p is 1, 2, or 3;
provided that no more than two of X^{2a}, X³a, X^{4a}, X^{5a}, and X^{6a} are N or N⁺—O;
provided that no more than one of X^{3c}, X^{4c}, X^{5c}, and X^{6c} is N; and
provided that R^{4a} is not CH(OH)—R^{4a}, wherein when R^{4a′} is H or C₁-C₅ alkyl optionally substituted by one or more halo, —OR¹¹, 3-7 membered heterocycloalkyl, —NR⁹R¹⁰, C₁-C₆ alkyl, or —S(O)₂R⁷.

2. The compound of clause 1, wherein the compound has formula (I-A)

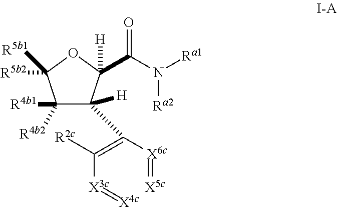

or a pharmaceutically acceptable salt thereof.

3. The compound of clause 1, wherein the compound has formula (I-A-1)

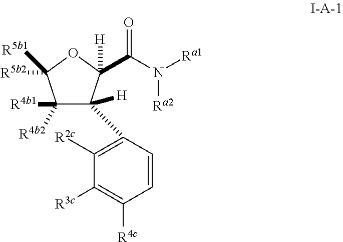

or a pharmaceutically acceptable salt thereof.

4. The compound of clause 1, wherein the compound has formula (I-B)

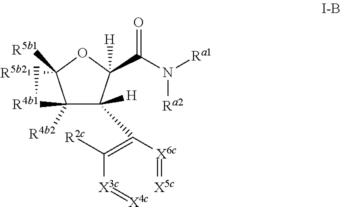

or a pharmaceutically acceptable salt thereof.

5. The compound of clause 1, wherein the compound has formula (I-B-1)

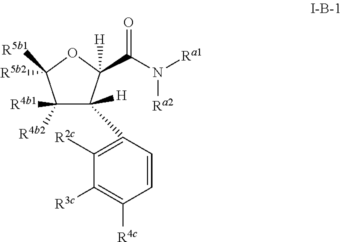

or a pharmaceutically acceptable salt thereof.

6. The compound of any one of clauses 1-5, or the pharmaceutically acceptable salt thereof, wherein R^{a1} is

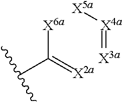

### and R^{a2} is H.

7. The compound of any one of clauses 1-5, or the pharmaceutically acceptable salt thereof, wherein R^{a1} is

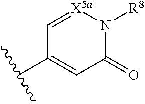

### and R^{a2} is H.

8. The compound of any one of clauses 1-5, or the pharmaceutically acceptable salt thereof, wherein R^{a1} is

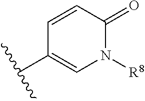

### and R^{a2} is H.

9. The compound of any one of clauses 1-5, or the pharmaceutically acceptable salt thereof, wherein R^{a1} is a 5-membered heteroaryl, a 9-10 membered aryl, or a 9-10 membered heteroaryl, wherein the 5-membered heteroaryl, 9-10 membered aryl, or 9-10 membered heteroaryl is optionally substituted by one or more R^{a3}; and R^{a2} is H.

10. The compound of clause 6, or the pharmaceutically acceptable salt thereof, wherein X^{2a} is C—R^{2a} and R^{2a} is H, X^{5a} C—R^{5a} and R^{5a} is H, and X^{6a} is C—R^{6a} and R^{6a} is H.

11. The compound of any one of clauses 1-6 or 10, or the pharmaceutically acceptable salt thereof, wherein X^{3a} is N or C—R^{3a}, wherein R^{3a} is —OR¹¹, —COOH, —S(O)₂R⁷, —S(O)(NR⁹)R⁷, —S(O)NR⁹R¹⁰, or —S(O)R⁷.

12. The compound of any one of clauses 1-6, 10, or 11, or the pharmaceutically acceptable salt thereof, wherein X^{4a} is N.

13. The compound of any one of clauses 1-5, 7, or 8, or the pharmaceutically acceptable salt thereof, wherein X^{5a} is C—R^{5a} and R^{5a} is H.

14. The compound of any one of clauses 1-5 or 9, or the pharmaceutically acceptable salt thereof, wherein R^{a1} is a 5-membered heteroaryl or a 9-10 membered heteroaryl, wherein the 5-membered heteroaryl or 9-10 membered heteroaryl is optionally substituted by one or more R^{a3}, and R^{a2} is H.

15. The compound of any one of clauses 1-6, 9-12, or 14, or the pharmaceutically acceptable salt thereof, wherein R⁷ is methyl, and R⁸ is H or methyl.

16. The compound of any one of clauses 1-15, or the pharmaceutically acceptable salt thereof, wherein R^{2c} is CH₃ or OCH₃.

17. The compound of any one of clauses 1-16, or a pharmaceutically acceptable salt thereof, wherein R^{3c} is halo or C₁-C₆ alkyl.

18. The compound of clause 17, or a pharmaceutically acceptable salt thereof, wherein R^{3c} is F.

19. The compound of clause 17, or a pharmaceutically acceptable salt thereof, wherein R^{3c} is CH₃.

20. The compound of any one of clauses 1-19, or a pharmaceutically acceptable salt thereof, wherein R^{4c} is halo.

21. The compound of clause 20, or a pharmaceutically acceptable salt thereof, wherein R^{4c} is F.

22. The compound of any one of clauses 1-21, or a pharmaceutically acceptable salt thereof, wherein R^{5c} is H.

23. The compound of any one of clauses 1-22, or a pharmaceutically acceptable salt thereof, wherein R^{6c} is H.

24. The compounds of any one of clauses 1-23, or a pharmaceutically acceptable salt thereof, wherein one of R^{4b}i and R^{4b2} is H and one is methyl.

25. The compounds of any one of clauses 1-23, or a pharmaceutically acceptable salt thereof, wherein one of R^{5b1} and R^{5b2} is methyl and one is trifluoromethyl.

26. A compound selected from Table A, or a pharmaceutically acceptable salt thereof.

27. The compound of any one of clauses 1-26 in non-salt form.

28. A pharmaceutical composition comprising a therapeutically effective amount of the compound of any one of clauses 1-26, or a pharmaceutically acceptable salt thereof, or the compound of clause 27 and one or more pharmaceutically acceptable carriers or vehicles.

29. A pharmaceutical composition comprising the compound of any one of clauses 1-26, or a pharmaceutically acceptable salt thereof, or the compound of clause 27 and one or more pharmaceutically acceptable carriers or vehicles.

30. A method of inhibiting a voltage-gated sodium channel in a subject comprising administering to the subject the compound of any one of clauses 1-26, or a pharmaceutically acceptable salt thereof, the compound of clause 27, or the pharmaceutical composition of clause 28 or 29.

31. The method of clause 30, wherein the voltage-gated sodium channel is Na_{V}1.8.

32. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of any one of clauses 1-26, or a pharmaceutically acceptable salt thereof, the compound of clause 27, or the pharmaceutical composition of clause 28 or 29.

33. The method of clause 32, where the method comprises treating or lessening the severity in the subject of neuropathic pain.

34. The method of clause 33, wherein the neuropathic pain comprises post-herpetic neuralgia.

35. The method of clause 33, wherein the neuropathic pain comprises small-fiber neuropathy.

36. The method of clause 33, wherein the neuropathic pain comprises idiopathic small-fiber neuropathy.

37. The method of clause 33, wherein the neuropathic pain comprises diabetic neuropathy.

38. The method of clause 32, wherein the diabetic neuropathy comprises diabetic peripheral neuropathy.

39. The method of clause 32, wherein the method comprises treating or lessening the severity in the subject of musculoskeletal pain.

40. The method of clause 39, wherein the musculoskeletal pain comprises osteoarthritis pain.

41. The method of clause 32, wherein the method comprises treating or lessening the severity in the subject of acute pain.

42. The method of clause 41, wherein the acute pain comprises acute post-operative pain.

43. The method of clause 32, wherein the method comprises treating or lessening the severity in the subject of postsurgical pain.

44. The method of clause 43, wherein the postsurgical pain comprises bunionectomy pain.

45. The method of clause 43, wherein the postsurgical pain comprises abdominoplasty pain.

46. The method of clause 43, wherein the postsurgical pain comprises herniorrhaphy pain.

47. The method of clause 32, wherein the method comprises treating or lessening the severity in the subject of visceral pain.

48. The method of any one of clauses 30-47, wherein said subject is treated with one or more additional therapeutic agents administered concurrently with, prior to, or subsequent to treatment with the compound, pharmaceutically acceptable salt, or pharmaceutical composition.

49. Use of the compound of any one of clauses 1-26, or a pharmaceutically acceptable salt thereof, the compound of clause 27, or the pharmaceutical composition of clause 28 or 29, as a medicament.

### EXAMPLES

General methods. ¹H NMR spectra were obtained as solutions in an appropriate deuterated solvent such as dimethyl sulfoxide-d₆ (DMSO-d6).

Compound purity, retention time, and electrospray mass spectrometry (ESI-MS) data were determined by LC/MS analysis. LC/MS analysis was conducted using an Acquity UPLC BEH C₈ column (50×2.1 mm, 1.7 m particle) made by Waters (pn: 186002877) with a (2.1×5 mm, 1.7 μm particle) guard column (pn: 186003978), and a dual gradient run from 2-98% mobile phase B over 4.45 minutes. Mobile phase A=H₂O (10 mM ammonium formate with 0.05% ammonium hydroxide). Mobile phase B=acetonitrile. Flow rate=0.6 mL/min, injection volume=2 μL, and column temperature=45° C.

X-ray powder diffraction analysis: X-ray powder diffraction (XRPD) analysis was performed at room temperature in transmission mode using a PANalytical Empyrean system equipped with a sealed tube source and a PIXcel 3D Medipix-3 detector (Malvern PANalytical Inc, Westborough, Massachusetts). The X-Ray generator operated at a voltage of 45 kV and a current of 40 mA with copper radiation (1.54060 A). The powder sample was placed on a 96 well sample holder with mylar film and loaded into the instrument. The sample was scanned over the range of about 3° to about 40°2θ with a step size of 0.0131303° and 49s per step.

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| UPLC | Ultra performance liquid chromatography |
| HPLC/MS/MS | High performance liquid chromatography/tandem |
| | mass spectrometry |
| IS | Internal standard |
| HPLC | High performance liquid chromatography |
| SCX | Strong Cation Exchange |
| SFC | Supercritical fluid chromatography |
| ESI | Electrospray ionization |
| g | Grams |
| mg | Milligrams |
| L | Liter(s) |
| mL | Milliliters |
| μL | Microliters |
| nL | Nanoliters |
| mmol | Millimoles |
| hr, h | Hours |
| min | Minutes |
| ms | Millisecond |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/v | Weight-volume concentration |
| AcOH | Acetic acid |
| K^{t}OBu | Potassium tert-butoxide |
| t-BuOH | tert-butyl alcohol |
| Boc₂O | Di-tert-butyl dicarbonate |
| Cbz | Carboxybenzyl |
| CDI | 1,1′-Carbonyldiimidazole |
| DAST | Diethylaminosulfur trifluoride |
| DCM | Dichloromethane |
| DCE | Dichloroethane |
| DIPEA | N,N-Diisopropyl ethyl amine |
| DIAD | Diisopropyl azodicarboxylate |
| DMA | N,N-Dimethylacetamide |
| DMAP | 4-(Dimethylamino)pyridine |
| DMF | N,N-Dimethylformamide |
| DMSO | Dimethyl sulfoxide |
| DRG | Dorsal root ganglia |
| EDCI | 1-Ethyl-3-(3′-dimethylaminopropyl)carbodiimide |
| EtOH | Ethanol |
| EtOAc | Ethyl acetate |
| HATU | 1-[Bis(dimethylamino)methylene]-1H-1,2,3-triazolo[4,5-b] |
| | pyridinium 3-oxide hexafluorophosphate |
| IPA | Isopropylamine |
| T3P | Propylphosphonic anhydride, i.e., 2,4,6-tripropyl-1,3,5,2,4,6- |
| | trioxatriphosphinane 2,4,6-trioxide, 1-Propanephosphonic |
| | anhydride |
| LDA | Lithium diisopropylamide |
| m-CPBA | 3-Chloroperbenzoic acid |
| MeOH | Methanol |
| MeCN | Acetonitrile |
| MsCl | Methanesulfonyl chloride |
| MTBE | Methyl tert-butyl ether |
| NBS | N-Bromosuccinimide |
| NMP | N-Methylpyrrolidone |
| PTSA | p-Toluenesulfonic acid |
| THF | Tetrahydrofuran |
| TBAB | Tetrabutylammonium bromide |
| TBAF | Tetrabutylammonium fluoride |
| TBDPS | tert-butyldiphenylsilyl |
| TCHF | N,N,N′,N′-tetramethylchloroformamidinium |
| | hexafluorophosphate |
| TEA | triethylamine |
| TIPS | Triisopropylsilyl |
| TFA | Trifluoroacetic acid |
| TFAA | Trifluoracetic anhydride |
| RT | Room temperature |
| r.t. | Retention time |
| E-VIPR | Electrical stimulation voltage ion probe reader |
| HEK | Human embryonic kidney |
| KIR2.1 | Inward-rectifier potassium ion channel 2.1 |
| DMEM | Dulbecco′s Modified Eagle′s Medium |
| FBS | Fetal bovine serum |
| NEAA | Non-essential amino acids |
| HEPES | 2-[4-(2-hydroxyethyl)piperazin-1-yl]ethanesulfonic acid |
| DiSBAC₆(3) | Bis-(1,3-dihexyl-thiobarbituric acid) trimethine oxonol |
| CC2-DMPE | Chlorocoumarin-2-dimyristoyl phosphatidylethanolamine |
| VABSC-1 | Voltage Assay Background Suppression Compound |
| HS | Human serum |
| BSA | Bovine Serum Albumin |
| SEMCI | 2-(trimethylsilyl)ethoxymethyl choride |
| STAB | Sodium triacetoxyborohydride |
| TMS | Trimethyl silyl |
| TBS/TBDMS | Tert-butyl dimethylsilyl |
| Ph | Phenyl |
| Ts | Tosyl |
| tBuBrettPhos-Pd-G3 | [(2-Di-tert-butylphosphino-3,6-dimethoxy-2′,4′,6′-triisopropyl- |
| | 1,1′-biphenyl)-2-(2′-amino-1,1′-biphenyl)]palladium(II) |
| | methanesulfonate |

General Method A: m-CPBA N-oxide formation (e.g., 2)

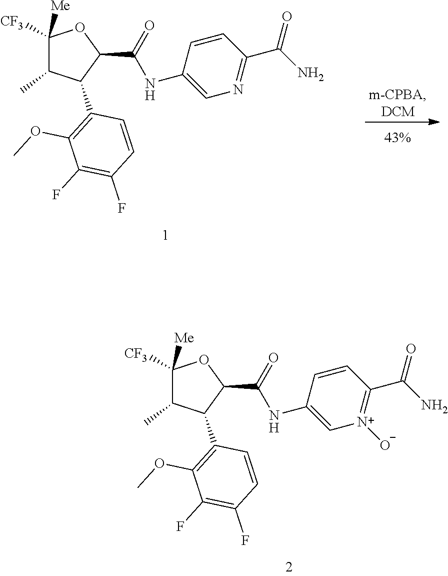 [SMILES: COc1c([C@H]2[C@H](C(=O)Nc3ccc(C(N)=O)nc3)O[C@@](C)(C(F)(F)F)[C@H]2C)ccc(F)c1F ; COc1c([C@H]2[C@H](C(=O)Nc3ccc(C(N)=O)[n+]([O-])c3)O[C@@](C)(C(F)(F)F)[C@H]2C)ccc(F)c1F]

To a solution of 5-((2R,3S,4S,5R)-3-(3,4-difluoro-2-methoxyphenyl)-4,5-dimethyl-5-(trifluoromethyl)tetrahydrofuran-2-carboxamido)picolinamide (1, 33 mg, 0.06971 mmol) in DCM (2 mL) was added m-CPBA (55 mg, 0.2390 mmol). The reaction was stirred at ambient temperature overnight. Further m-CPBA (55 mg, 0.2390 mmol) was added and the reaction stirred at ambient temperature overnight. The reaction was diluted with EtOAc and quenched with saturated aqueous NaHCO₃ solution. The aqueous layer was washed EtOAc. The combined organics were washed with brine and dried with MgSO₄. The crude product was purified by flash chromatography (0-100% EtOAc in heptane) and then further purified by preparative reverse phase HPLC (basic eluent) to afford 2-carbamoyl-5-((2R,3S,4S,5R)-3-(3,4-difluoro-2-methoxyphenyl)-4,5-dimethyl-5-(trifluoromethyl)tetra hydrofuran-2-carboxamido)pyridine 1-oxide (2, 14.9 mg, 43%) as a white solid. ¹H NMR (500 MHz, DMSO-d₆) δ 10.70 (s, 1H), 10.11 (d, J=4.5 Hz, 1H), 8.86 (d, J=2.0 Hz, 1H), 8.16 (d, J=9.0 Hz, 1H), 8.13 (d, J=4.3 Hz, 1H), 7.73 (dd, J=9.0, 2.0 Hz, 1H), 7.21-7.12 (m, 2H), 5.12 (d, J=10.1 Hz, 1H), 4.25 (dd, J=10.1, 7.7 Hz, 1H), 3.94 (d, J=2.0 Hz, 3H), 2.77 (p, J=7.5 Hz, 1H), 1.60 (s, 3H), 0.78-0.66 (m, 3H) ppm. ESI-MS m/z calc. 489.13232, found 490.2 (M+1)⁺; 488.1 (M−1)⁻; Retention time: 3.17 minutes.

### General Method B: TFA Deprotection of Ketals to Give Diols (e.g., 3)

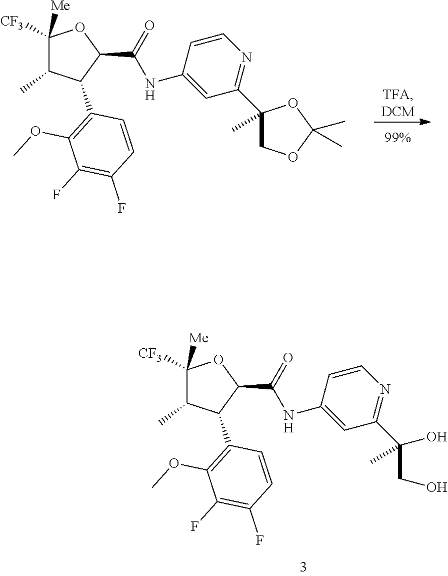

A solution of rel-(2R*,3S*,4S*,5R*)-3-(3,4-difluoro-2-methoxyphenyl)-4,5-dimethyl-5-(trifluoromethyl)-N-(2-(2,2,4-trimethyl-1,3-dioxolan-4-yl)pyridin-4-yl)tetrahydrofuran-2-carboxamide (1.190 g, 2.185 mmol) in DCM (20 mL) and TFA (5 mL, 64.90 mmol) was stirred at ambient temperature. Upon completion the mixture was washed with 1M NaOH (2×50 mL), dried (MgSO₄) and concentrated in vacuo. The residue was dissolved in MeCN and H₂O, (3:1) and freeze-dried to give rel-(2R*,3S*,4S*,5R*)-3-(3,4-difluoro-2-methoxyphenyl)-N-(2-(1,2-dihydroxypropan-2-yl)pyridin-4-yl)-4,5-dimethyl-5-(trifluoromethyl)tetrahydrofuran-2-carboxamide (3, 1.0886 g, 99%) as an amorphous white solid. ¹H NMR (400 MHz, DMSO-d₆) δ 10.57 (s, 1H), 8.35 (d, J=5.3 Hz, 1H), 7.84 (s, 1H), 7.52 (s, 1H), 7.24-7.06 (m, 2H), 5.09 (d, J=10.0 Hz, 1H), 5.05 (s, 1H), 4.59 (s, 1H), 4.25 (dd, J=10.4, 7.8 Hz, 1H), 3.95 (d, J=2.3 Hz, 3H), 3.50 (d, J=5.1 Hz, 2H), 2.85-2.70 (m, 1H), 1.60 (s, 3H), 1.34 (s, 3H), 0.72 (d, J=5.6 Hz, 3H) ppm; ESI-MS m/z calc. 504.16837, found 505.3 (M+1)⁺; 503.5 (M−1)⁻.

### General Method D: Oxidation of Thioether to Sulfone (e.g., 4)

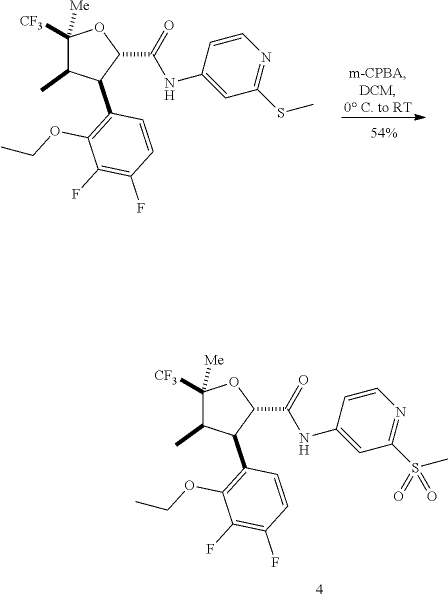

To a solution of rel-(2S,3R,4R,5S)-3-(2-ethoxy-3,4-difluoro-phenyl)-4,5-dimethyl-N-(2-methylsulfanyl-4-pyridyl)-5-(trifluoromethyl)tetrahydrofuran-2-carboxamide (46 mg, 0.094 mmol) in dichloromethane (3 mL), stirring at 0° C., was added m-CPBA (53 mg, 0.24 mmol) in one portion. The reaction was stirred at 0° C. for 1 hour then raised to ambient temperature and stirred for a further 2 hours. The reaction was quenched with saturated sodium bicarbonate solution and extracted with DCM (×3). The combined organic extracts were passed through a phase separator cartridge and the filtrate was concentrated in vacuo. The residue was purified by flash column chromatography (4 g SiO₂, 0 to 100% EtOAc/Heptane, loaded in DCM onto Telos nm) to give a yellow oil. The oil was repurified by preparative reverse phase HPLC (basic eluent) to give rel-(2S,3R,4R,5S)-3-(2-ethoxy-3,4-difluorophenyl)-4,5-dimethyl-N-(2-(methylsulfonyl)pyridin-4-yl)-5-(trifluoromethyl)tetrahydrofuran-2-carboxamide (4, 26.8 mg, 54%) as an off-white solid. ¹H NMR (500 MHz, DMSO-d₆) δ 10.92 (s, 1H), 8.65 (d, J=5.5 Hz, 1H), 8.38 (d, J=2.0 Hz, 1H), 7.91 (dd, J=5.5, 2.1 Hz, 1H), 7.22-7.14 (m, 2H), 5.14 (d, J=10.4 Hz, 1H), 4.29 (dd, J=10.4, 7.5 Hz, 1H), 4.25-4.12 (m, 2H), 3.25 (s, 3H), 2.77 (p, J=7.5 Hz, 1H), 1.62 (s, 3H), 1.35 (t, J=7.0 Hz, 3H), 0.78-0.70 (m, 3H) ppm. ESI-MS m/z calc. 522.12476, found 523.5 (M+1)⁺; 521.5 (M−1)⁻.

### General Method E: Formic Acid Deprotection of Ketals to Give Diols (e.g., 5)

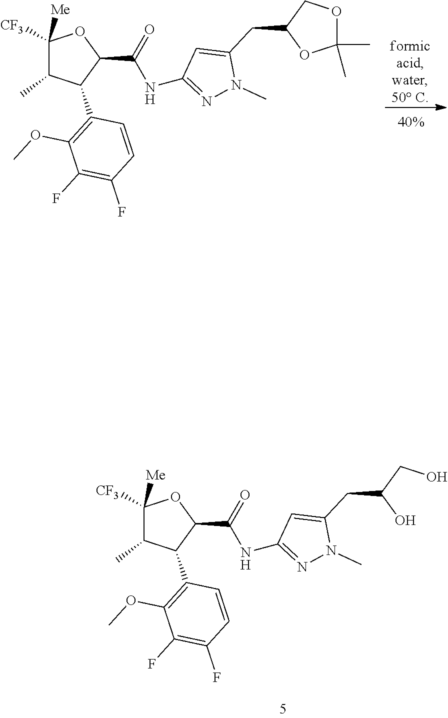

To a solution of (2R,3S,4S,5R)-3-(3,4-difluoro-2-methoxyphenyl)-N-(5-(((S)-2,2-dimethyl-1,3-dioxolan-4-yl)methyl)-1-methyl-1H-pyrazol-3-yl)-4,5-dimethyl-5-(trifluoromethyl)tetrahydrofuran-2-carboxamide (80 mg, 0.1461 mmol) in formic acid (551 μL, 14.61 mmol) was added water (1 mL) and the mixture was heated to 50° C. for 1 hour. The reaction mixture was cooled down and concentrated to dryness. The product was purified directly by preparative reverse phase HPLC (basic eluent) and freeze-dried to give (2R,3S,4S,5R)-3-(3,4-difluoro-2-methoxyphenyl)-N-(5-((S)-2,3-dihydroxypropyl)-1-methyl-1H-pyrazol-3-yl)-4,5-dimethyl-5-(trifluoromethyl)tetrahydrofuran-2-carboxamide (5, 30 mg, 40%) as a white solid. ¹H NMR (500 MHz, DMSO-d₆) δ 10.58 (s, 1H), 7.26-7.00 (m, 2H), 6.32 (s, 1H), 5.06 (d, J=10.7 Hz, 1H), 4.74 (d, J=5.2 Hz, 1H), 4.62 (t, J=5.6 Hz, 1H), 4.21 (dd, J=10.7, 7.5 Hz, 1H), 3.95 (d, J=2.0 Hz, 3H), 3.64 (s, 3H), 3.26 (td, J=11.7, 10.8, 6.3 Hz, 1H), 2.78-2.66 (m, 2H), 2.58-2.52 (m, 1H), 1.58 (s, 3H), 0.72-0.63 (m, 3H) ppm. ESI-MS m/z calc. 507.17926, found 508.4 (M+1)⁺; Retention time: 2.96 minutes.

### General Method F: Oxidation of Thioether to Sulfoxide (e.g., 6, 7, 8 and 9)

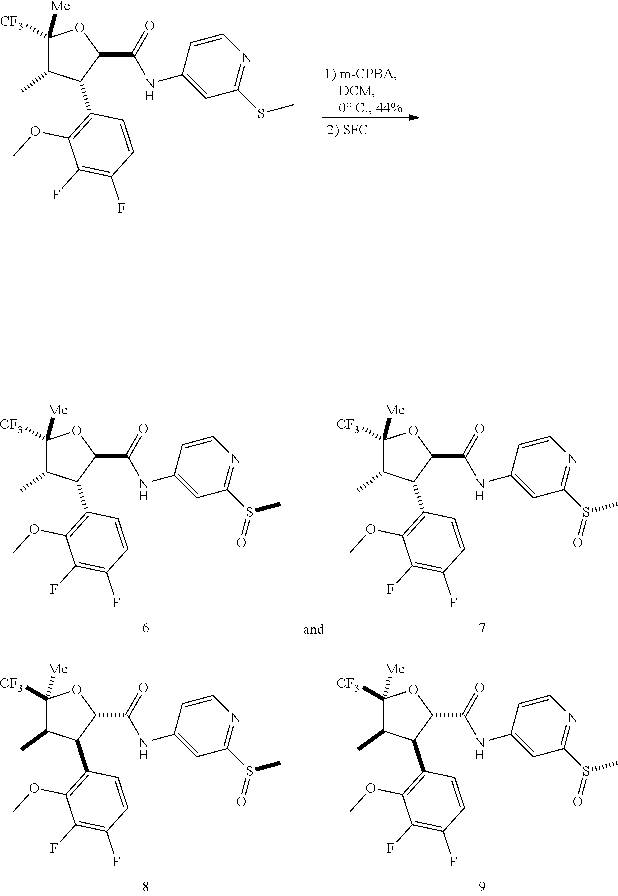

### Step 1:

To a solution of rac-(2S,3R,4R,5S)-3-(3,4-difluoro-2-methoxy-phenyl)-4,5-dimethyl-N-(2-methylsulfanyl-4-pyridyl)-5-(trifluoromethyl)tetrahydrofuran-2-carboxamide (86 mg, 0.18 mmol) in DCM (2 mL), stirring at 0° C., was added m-CPBA (40 mg, 0.16 mmol). The reaction was stirred for 10 minutes before a further portion of m-CPBA (10 mg) was added. After a further 10 minutes the reaction was quenched with saturated aqueous NaHCO₃ solution and diluted with DCM. The mixture was passed through a phase separator cartridge, washing the aqueous layer with DCM. The filtrate was concentrated in vacuo. Purification by reverse phase preparative HPLC (basic eluent) gave rac-(2S,3R,4R,5S)-3-(3,4-difluoro-2-methoxyphenyl)-4,5-dimethyl-N-(2-(methylsulfinyl)pyridin-4-yl)-5-(trifluoromethyl)tetrahydrofuran-2-carboxamide (39.2 mg, 44%). ESI-MS m/z calc. 492.11423, found 493.1 (M+1)⁺; 491.3 (M−1)⁻.

### Step 2:

rac-(2S,3R,4R,5S)-3-(3,4-difluoro-2-methoxyphenyl)-4,5-dimethyl-N-(2-(methylsulfinyl)pyridin-4-yl)-5-(trifluoromethyl)tetrahydrofuran-2-carboxamide (39.2 mg, 0.080 mmol) was purified by chiral SFC. First using a Chiralpak AS-H column, 5 μm particle size, 25 cm×10 mm from Daicel on a Minigram SFC instrument from Berger Instruments to separate the two diastereomers and a second time using an (R,R)-Whelk-01 columns, 5 μm particle size, 25 cm×21.2 mm from Regis Technologies to isolate the individual enantiomers, to give:

First eluting isomer from separation 1, first eluting isomer from separation 2: rel-(2S,3R,4R,5S)-3-(3,4-difluoro-2-methoxyphenyl)-4,5-dimethyl-N-(2-(methylsulfinyl)pyridin-4-yl)-5-(trifluoromethyl)tetrahydrofuran-2-carboxamide (6, 8 mg). ¹H NMR (500 MHz, Chloroform-d) δ 8.91 (s, 1H), 8.49 (d, J=5.5 Hz, 1H), 8.09 (dd, J=5.5, 2.1 Hz, 1H), 7.82 (d, J=2.0 Hz, 1H), 7.07 (ddd, J=8.0, 5.5, 2.0 Hz, 1H), 6.90 (td, J=9.2, 7.4 Hz, 1H), 5.03 (dd, J=11.0, 3.0 Hz, 1H), 4.10 (dd, J=11.0, 8.1 Hz, 1H), 4.01 (d, J=2.8 Hz, 3H), 2.85 (s, 3H), 2.76 (p, J=7.7 Hz, 1H), 1.69 (d, J=1.5 Hz, 3H), 0.80 (dq, J=7.3, 2.3 Hz, 3H); ESI-MS m/z calc. 492.11423, found 493.2 (M+1)⁺; 491.2 (M−1)⁻.

First eluting isomer from separation 1, Second eluting isomer from separation 2: rel-(2S,3R,4R,5S)-3-(3,4-difluoro-2-methoxyphenyl)-4,5-dimethyl-N-(2-(methylsulfinyl)pyridin-4-yl)-5-(trifluoromethyl)tetrahydrofuran-2-carboxamide (7, 6 mg). ¹H NMR (500 MHz, Chloroform-d) δ 8.49 (d, J=5.5 Hz, 1H), 8.06 (dd, J=5.5, 2.2 Hz, 1H), 7.80 (d, J=2.1 Hz, 1H), 7.06 (ddd, J=8.0, 5.4, 2.0 Hz, 1H), 6.90 (td, J=9.2, 7.4 Hz, 1H), 5.03 (d, J=11.0 Hz, 1H), 4.10 (dd, J=11.0, 8.1 Hz, 1H), 4.01 (d, J=2.8 Hz, 3H), 2.85 (s, 3H), 2.75 (p, J=7.7 Hz, 1H), 1.69 (d, J=1.3 Hz, 3H), 0.87-0.76 (m, 3H) ppm; ESI-MS m/z calc. 492.11423, found 493.2 (M+1)⁺; 491.3 (M−1)⁻.

Second eluting isomer from separation 1, first eluting isomer from separation 2: rel-(2R,3S,4S,5R)-3-(3,4-difluoro-2-methoxyphenyl)-4,5-dimethyl-N-(2-(methylsulfinyl)pyridin-4-yl)-5-(trifluoromethyl)tetrahydrofuran-2-carboxamide (8, 8 mg). ¹H NMR (500 MHz, Chloroform-d) δ 8.88 (d, J=8.2 Hz, 1H), 8.49 (d, J=5.5 Hz, 1H), 8.07 (ddd, J=9.3, 5.5, 2.2 Hz, 1H), 7.80 (d, J=2.1 Hz, 1H), 7.12-7.03 (m, 1H), 6.90 (td, J=9.2, 7.4 Hz, 1H), 5.03 (dd, J=11.0, 3.0 Hz, 1H), 4.10 (ddd, J=10.4, 8.2, 1.7 Hz, 1H), 4.01 (d, J=2.7 Hz, 3H), 2.85 (d, J=2.5 Hz, 3H), 2.79-2.69 (m, 1H), 1.72-1.66 (m, 3H), 0.80 (dq, J=7.4, 2.4 Hz, 3H) ppm; ESI-MS m/z calc. 492.11423, found 493.1 (M+1)⁺; 491.2 (M−1)⁻.

Second eluting isomer from separation 1, second eluting isomer from separation 2: rel-(2R,3S,4S,5R)-3-(3,4-difluoro-2-methoxyphenyl)-4,5-dimethyl-N-(2-(methylsulfinyl)pyridin-4-yl)-5-(trifluoromethyl)tetrahydrofuran-2-carboxamide (9, 8 mg). ¹H NMR (500 MHz, Chloroform-d) δ 8.85 (s, 1H), 8.49 (d, J=5.5 Hz, 1H), 8.06 (dd, J=5.5, 2.2 Hz, 1H), 7.79 (d, J=2.1 Hz, 1H), 7.07 (ddd, J=8.2, 5.4, 2.0 Hz, 1H), 6.90 (td, J=9.2, 7.4 Hz, 1H), 5.03 (d, J=11.0 Hz, 1H), 4.10 (dd, J=11.1, 8.1 Hz, 1H), 4.01 (d, J=2.8 Hz, 3H), 2.85 (d, J=3.1 Hz, 3H), 2.76 (p, J=7.6 Hz, 1H), 1.69 (d, J=1.4 Hz, 3H), 0.79 (dt, J=7.5, 2.3 Hz, 3H) ppm; ESI-MS m/z calc. 492.11423, found 493.1 (M+1)⁺; 491.2 (M−1)⁻.

### General Method G: Sulfoximine Formation by Thioether Oxidation (e.g., 10 and 11)

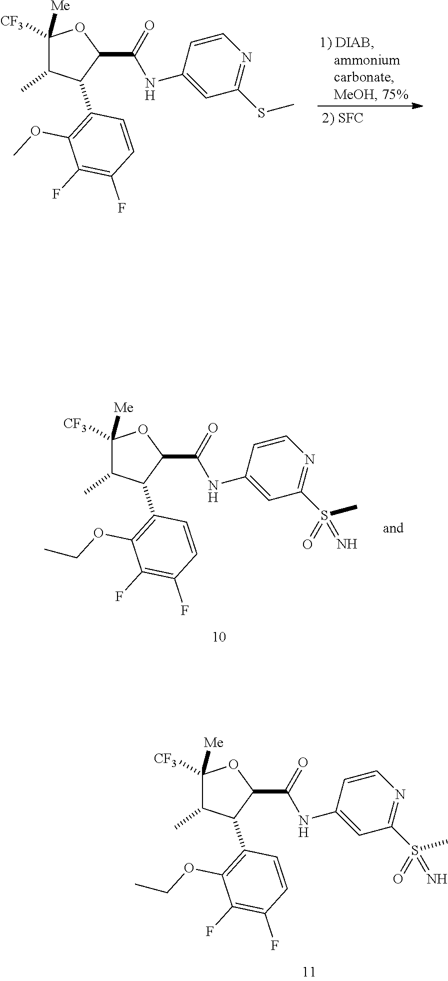

### Step 1:

To a solution of rel-(2S,3R,4R,5S)-3-(2-ethoxy-3,4-difluoro-phenyl)-4,5-dimethyl-N-(2-methylsulfanyl-4-pyridyl)-5-(trifluoromethyl)tetrahydrofuran-2-carboxamide (85 mg, 0.16 mmol) in MeOH (5 mL) was added (diacetoxyiodo)benzene (145 mg, 0.45 mmol) and ammonium carbamate (28 mg, 0.36 mmol). The reaction was stirred at ambient temperature for 5 hours before being concentrated in vacuo. The residue was partitioned between DCM and saturated aqueous Na₂CO₃, the layers were separated and the aqueous layer was extracted with DCM (×3). The combined organic layers were passed through a phase separator cartridge and concentrated in vacuo. The residue purified by flash column chromatography (4 g SiO₂, 0 to 100% EtOAc in heptane, loaded in DCM on Telos nM) to give rel-(2S,3R,4R,5S)-3-(2-ethoxy-3,4-difluoro-phenyl)-4,5-dimethyl-N-[2-(methylsulfonimidoyl)-4-pyridyl]-5-(trifluoromethyl)tetrahydrofuran-2-carboxamide (65.8 mg, 75%). ¹H NMR (500 MHz, DMSO-d₆) δ 10.89 (s, 1H), 8.58 (d, J=5.5 Hz, 1H), 8.36 (dd, J=4.3, 2.0 Hz, 1H), 7.82 (ddd, J=5.7, 3.8, 2.1 Hz, 1H), 7.22-7.14 (m, 2H), 5.12 (d, J=10.4 Hz, 1H), 4.37-4.27 (m, 2H), 4.26-4.12 (m, 2H), 3.12 (d, J=1.0 Hz, 3H), 2.77 (p, J=7.4 Hz, 1H), 1.62 (s, 3H), 1.36 (t, J=7.0 Hz, 3H), 0.79-0.69 (m, 3H) ppm; ESI-MS m/z calc. 521.14075, found 522.6 (M+1)⁺; 520.6 (M−1)⁻.

### Step 2:

rel-(2S,3R,4R,5S)-3-(2-ethoxy-3,4-difluoro-phenyl)-4,5-dimethyl-N-[2-(methylsulfonimidoyl)-4-pyridyl]-5-(trifluoromethyl)tetrahydrofuran-2-carboxamide (63.8 mg, 0.1165 mmol) was purified by chiral SFC [System: (R,R)-Whelk-01 column, 5 μm particle size, 25 cm×21.2 mm from Regis Technologies, MeOH, 20 mM NH₃] to give:

First eluting isomer (rt=5.04 minutes): rel-(2S,3R,4R,5S)-3-(2-ethoxy-3,4-difluorophenyl)-4,5-dimethyl-N-(2-(S-methylsulfonimidoyl)pyridin-4-yl)-5-(trifluoromethyl)tetrahydrofuran-2-carboxamide (10, 25 mg) as a white solid. ¹H NMR (500 MHz, DMSO-d₆) δ 10.89 (s, 1H), 8.57 (d, J=5.5 Hz, 1H), 8.36 (d, J=2.0 Hz, 1H), 7.81 (dd, J=5.5, 2.0 Hz, 1H), 7.22-7.11 (m, 2H), 5.12 (d, J=10.4 Hz, 1H), 4.36-4.24 (m, 2H), 4.24-4.10 (m, 2H), 3.12 (d, J=1.0 Hz, 3H), 2.76 (p, J=7.5 Hz, 1H), 1.62 (s, 3H), 1.36 (t, J=7.0 Hz, 3H), 0.78-0.67 (m, 3H) ppm; ESI-MS m/z calc. 521.14075, found 522.6 (M+1)⁺; 520.6 (M−1)

Second eluting isomer (rt=5.75 minutes): rel-(2S,3R,4R,5S)-3-(2-ethoxy-3,4-difluorophenyl)-4,5-dimethyl-N-(2-(S-methylsulfonimidoyl)pyridin-4-yl)-5-(trifluoromethyl)tetrahydrofuran-2-carboxamide (11, 25 mg) as a white solid. ¹H NMR (500 MHz, DMSO-d₆) δ 10.90 (s, 1H), 8.57 (d, J=5.5 Hz, 1H), 8.35 (d, J=2.0 Hz, 1H), 7.82 (dd, J=5.5, 2.1 Hz, 1H), 7.23-7.07 (m, 2H), 5.12 (d, J=10.4 Hz, 1H), 4.38-4.27 (m, 2H), 4.27-4.07 (m, 2H), 3.12 (d, J=1.1 Hz, 3H), 2.76 (p, J=7.5 Hz, 1H), 1.62 (s, 3H), 1.36 (t, J=7.0 Hz, 3H), 0.79-0.65 (m, 3H) ppm; ESI-MS m/z calc. 521.14075, found 522.5 (M+1)⁺; 520.6 (M−1)⁻.

### General Method H: Methylation of Sulfoximines (e.g., 12)

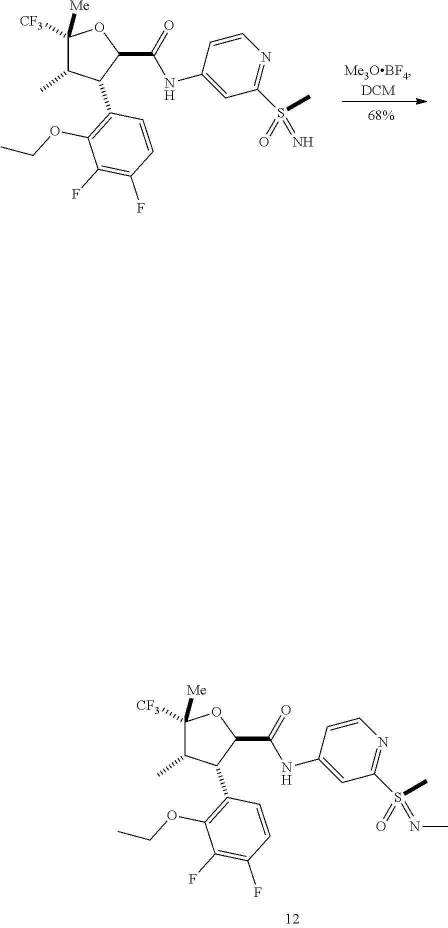

To a solution of rel-(2R,3S,4S,5R)-3-(2-ethoxy-3,4-difluoro-phenyl)-4,5-dimethyl-N-[2-(methylsulfonimidoyl)-4-pyridyl]-5-(trifluoromethyl)tetrahydrofuran-2-carboxamide (14.4 mg, 0.027 mmol) in DCM (2 mL), stirring at ambient temperature under a nitrogen atmosphere, was added trimethyloxonium tetrafluoroborate (5 mg, 0.034 mmol) portion-wise. The reaction mixture was stirred at this temperature overnight, before being quenched by addition of saturated aqueous sodium bicarbonate (5 mL). The layers were separated, the aqueous layer extracted with DCM (3×5 mL) and the combined organic layers passed through a phase separator cartridge. The filtrate was concentrated in vacuo. Purification by reverse phase preparative HPLC (basic eluent) gave rel-(2R,3S,4S,5R)—N-(2-(N,S-dimethylsulfonimidoyl)pyridin-4-yl)-3-(2-ethoxy-3,4-difluorophenyl)-4,5-dimethyl-5-(trifluoromethyl)tetrahydrofuran-2-carboxamide (12, 10.2 mg, 68%). ¹H NMR (500 MHz, DMSO-d₆) δ 10.87 (s, 1H), 8.60 (d, J=5.5 Hz, 1H), 8.31 (d, J=2.1 Hz, 1H), 7.83 (dd, J=5.5, 2.1 Hz, 1H), 7.23-7.08 (m, 2H), 5.10 (d, J=10.3 Hz, 1H), 4.28 (dd, J=10.5, 7.6 Hz, 1H), 4.16 (ddd, J=16.4, 8.1, 6.7 Hz, 2H), 3.14 (s, 3H), 2.75 (p, J=7.5 Hz, 1H), 2.44 (s, 3H), 1.60 (s, 3H), 1.34 (t, J=7.0 Hz, 3H), 0.72 (d, J=7.0 Hz, 3H) ppm; ¹⁹F NMR (471 MHz, DMSO-d₆) δ−73.37, −13 8.18 (d, J=22.5 Hz), −154.54 (d, J=22.2 Hz) ppm; ESI-MS m/z calc. 535.15643, found 537.5 (M+1)⁺; 534.5 (M−1)⁻.

### General Method I: Boc Deprotection Using TFA (e.g., 13)

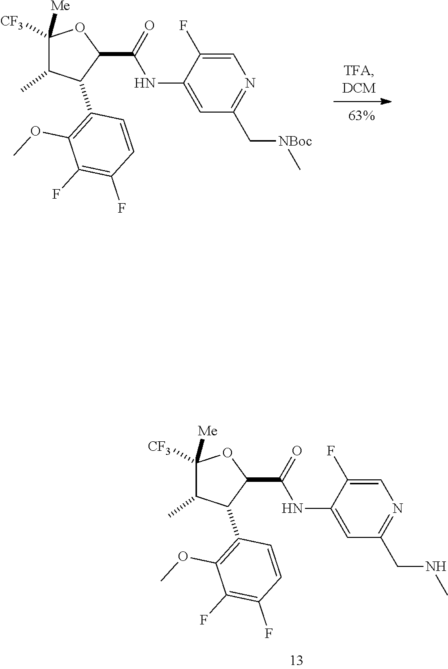

To a solution of rel-tert-butyl ((4-((2R,3S,4S,5R)-3-(3,4-difluoro-2-methoxyphenyl)-4,5-dimethyl-5-(trifluoromethyl)tetrahydrofuran-2-carboxamido)-5-fluoropyridin-2-yl)methyl)(methyl)carbamate (10.3 mg, 0.017 mmol) in DCM (1 mL), stirring at ambient temperature, was added TFA (50 μL, 0.65 mmol). The reaction was stirred for 72 hours then additional DCM (1 mL) and TFA (15 μL) was added. After 5 hours the reaction was concentrated in vacuo and passed through an SCX-2 cartridge, washing with MeOH and eluting the product with 2M methanolic ammonia. Purification by reverse phase preparative HPLC (basic elutent) gave rel-(2R,3S,4S,5R)-3-(3,4-difluoro-2-methoxyphenyl)-N-(5-fluoro-2-((methylamino)methyl)pyridin-4-yl)-4,5-dimethyl-5-(trifluoromethyl)tetrahydrofuran-2-carboxamide (13, 5.9 mg, 63%). ¹H NMR (500 MHz, Chloroform-d) δ 8.83 (s, 1H), 8.39 (s, 1H), 8.28 (d, J=6.2 Hz, 1H), 7.09 (t, J=7.1 Hz, 1H), 6.91 (q, J=8.7 Hz, 1H), 5.04 (d, J=11.0 Hz, 1H), 4.11-4.05 (m, 1H), 4.01 (d, J=2.7 Hz, 3H), 3.77 (s, 2H), 2.75 (q, J=7.6 Hz, 1H), 2.42 (s, 3H), 1.68 (s, 3H), 0.90-0.67 (m, 3H) ppm; ESI-MS m/z calc. 491.16437, found 492.9 (M+1)⁺.

General Method J: Deprotection of Silyl Groups with TBAF (e.g., 14)

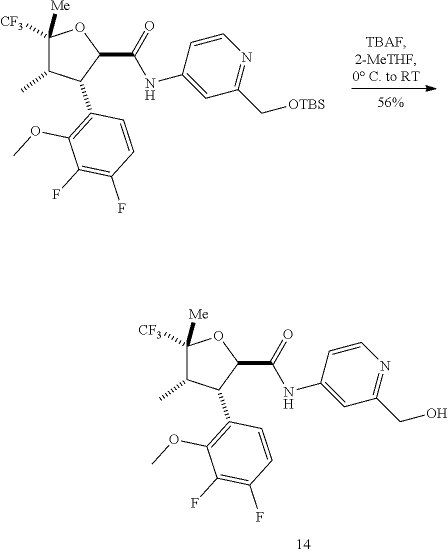 [SMILES: COc1c([C@H]2[C@H](C(=O)Nc3ccnc(CO[Si](C)(C)C(C)(C)C)c3)O[C@@](C)(C(F)(F)F)[C@H]2C)ccc(F)c1F ; COc1c([C@H]2[C@H](C(=O)Nc3ccnc(CO)c3)O[C@@](C)(C(F)(F)F)[C@H]2C)ccc(F)c1F]

A solution of TBAF in THF (300 μL of 1 M, 0.3000 mmol) was added to a stirred solution of rel-(2R,3S,4S,5R)—N-[2-[[tert-butyl(dimethyl)silyl]oxymethyl]-4-pyridyl]-3-(3,4-difluoro-2-methoxy-phenyl)-4,5-dimethyl-5-(trifluoromethyl)tetrahydrofuran-2-carboxamide (52 mg, 0.09049 mmol) in 2-methyltetrahydrofuran (5 mL) at 0° C. and the reaction stirred at ambient temperature for 2 hours. The reaction mixture was quenched with water (1 mL) and extracted with ethyl acetate (3×10 mL). The combined organic extracts were dried (MgSO₄) and concentrated in vacuo. The material was purified by preparative reverse phase HPLC (basic eluent). The fractions were collected and freeze-dried to give rel-(2R,3S,4S,5R)-3-(3,4-difluoro-2-methoxyphenyl)-N-(2-(hydroxymethyl)pyridin-4-yl)-4,5-dimethyl-5-(trifluoromethyl)tetrahydrofuran-2-carboxamide (14, 23.5 mg, 56%) as a white solid. ¹H NMR (500 MHz, DMSO-d₆) δ 10.58 (s, 1H), 8.33 (d, J=5.5 Hz, 1H), 7.71 (dd, J=2.1, 0.8 Hz, 1H), 7.50 (dd, J=5.5, 2.2 Hz, 1H), 7.20-7.12 (m, 2H), 5.39 (s, 1H), 5.09 (d, J=10.3 Hz, 1H), 4.50 (s, 2H), 4.25 (dd, J=10.3, 7.6 Hz, 1H), 3.96 (d, J=2.1 Hz, 3H), 2.78 (p, J=7.5 Hz, 1H), 1.60 (s, 3H), 0.74 (dd, J=7.5, 2.4 Hz, 3H) ppm; ¹⁹F NMR (471 MHz, DMSO-d₆) δ−73.38, −138.09 (d, J=21.1 Hz), −154.91 (d, J=21.3 Hz) ppm; ESI-MS m/z calc. 460.14215, found 461.7 (M+1); 459.7 (M−1)⁻; Retention time: 3.13 minutes.

### General Method K: N-Methylation Via Reductive Amination (e.g., 15)

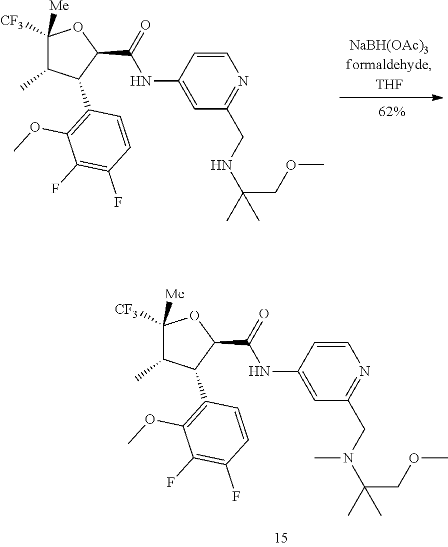

To a solution of (2R,3S,4S,5R)-3-(3,4-difluoro-2-methoxyphenyl)-N-(2-(((1-methoxy-2-methylpropan-2-yl)amino)methyl)pyridin-4-yl)-4,5-dimethyl-5-(trifluoromethyl)tetrahydrofuran-2-carboxamide (17.7 mg, 0.03244 mmol) in THF (1.0 mL) was added formaldehyde, 37% aqueous (104 μL, 3.775 mmol) then sodium triacetoxyborohydride (16 mg, 0.07585 mmol), followed by stirring at ambient temperature. The reaction mixture was diluted with EtOAc (2 mL), and washed with saturated aqueous NaHCO₃ (2 mL) and brine (2 mL), then loaded onto an SCX cartridge and washed with MeOH (10 mL) then 2M ammonia in MeOH (10 mL). The ammonia wash was concentrated under reduced pressure, then lyophilised to give (2R,3S,4S,5R)-3-(3,4-difluoro-2-methoxyphenyl)-N-(2-(((1-methoxy-2-methylpropan-2-yl)(methyl)amino)methyl)pyridin-4-yl)-4,5-dimethyl-5-(trifluoromethyl)tetrahydrofuran-2-carboxamide (15, 12.0 mg, 62%) as white solid. ¹H NMR (500 MHz, DMSO-d₆) δ 10.59 (s, 1H), 8.30 (s, 1H), 7.61 (s, 1H), 7.55 (dd, J=5.6, 2.1 Hz, 1H), 7.17 (td, J=9.5, 7.5 Hz, 1H), 7.13-7.07 (m, 1H), 5.08 (d, J=10.3 Hz, 1H), 4.25 (dd, J=10.3, 7.7 Hz, 1H), 3.95 (d, J=2.1 Hz, 3H), 3.64 (s, 2H), 3.29-3.23 (m, 6H), 2.77 (p, J=7.5 Hz, 1H), 2.10 (s, 2H), 1.59 (s, 3H), 1.07 (s, 6H), 0.76-0.68 (m, 3H) ppm. ESI-MS m/z calc. 559.24695, found 560.1 (M+1)⁺; Retention time: 2.97 minutes.

### General Method L: Amination of Esters (e.g., 1)

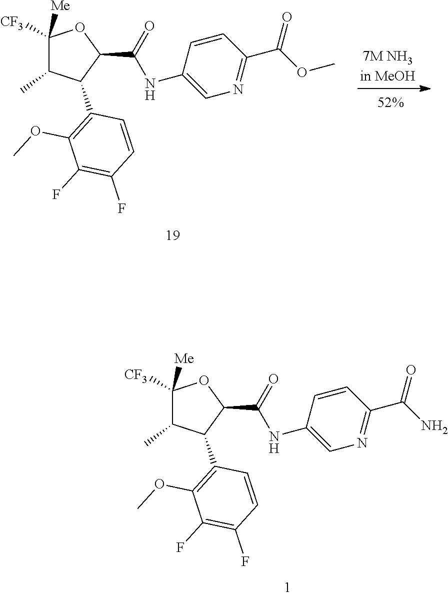

A solution of methyl 5-((2R,3S,4S,5R)-3-(3,4-difluoro-2-methoxyphenyl)-4,5-dimethyl-5-(trifluoromethyl)tetrahydrofuran-2-carboxamido)picolinic acid (19, 70 mg, 0.1276 mmol) in ammonia (7 M in methanol) (1 mL of 7 M, 7.0000 mmol) was stirred at room temperature overnight and then concentrated in vacuo to give a colorless oil. The crude material was purified by reverse phase chromatography (12 g C18, 30 to 80% acetonitrile containing 0.10% ammonium hydroxide in water containing 0.1% ammonium hydroxide) to give 5-((2R,3S,4S,5R)-3-(3,4-difluoro-2-methoxyphenyl)-4,5-dimethyl-5-(trifluoromethyl)tetrahydrofuran-2-carboxamido)picolinamide (1, 32 mg, 52%) as a white solid. ¹H NMR (400 MHz, Chloroform-d) δ 8.68 (q, J=1.0 Hz, 1H), 8.52 (s, 1H), 8.19-8.15 (m, 2H), 7.71 (d, J=14.0 Hz, 1H), 7.10-7.06 (m, 1H), 6.93-6.87 (m, 1H), 5.53 (s, 1H), 5.04 (d, J=11.0 Hz, 1H), 4.10 (dd, J=10.9, 7.9 Hz, 1H), 4.00 (d, J=2.7 Hz, 3H), 2.79-2.72 (m, 1H), 1.69 (s, 3H), 0.80-0.78 (m, 3H) ppm. ESI-MS m/z calc. 473.1374, found 474.1 (M+1)⁺; Retention time: 2.42 minutes.

General Method M: Deprotection of Silyl Groups with TFA (e.g., 17)

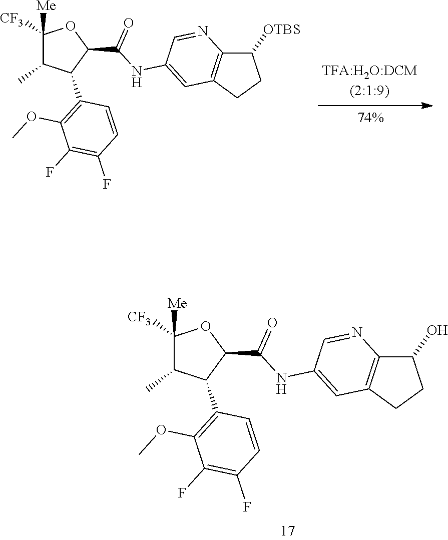 [SMILES: COc1c([C@H]2[C@H](C(=O)Nc3cnc4c(c3)CC[C@H]4O[Si](C)(C)C(C)(C)C)O[C@@](C)(C(F)(F)F)[C@H]2C)ccc(F)c1F ; COc1c([C@H]2[C@H](C(=O)Nc3cnc4c(c3)CC[C@H]4O)O[C@@](C)(C(F)(F)F)[C@H]2C)ccc(F)c1F]

rel-(2R*,3S*,4S*,5R*)-N-(7-((tert-butyldimethylsilyl)oxy)-6,7-dihydro-5H-cyclopenta[b]pyridin-3-yl)-3-(3,4-difluoro-2-methoxyphenyl)-4,5-dimethyl-5-(trifluoromethyl)tetrahydrofuran-2-carboxamide (13.5 mg, 0.02247 mmol) was dissolved in DCM (1.0 mL) and water (100 μL) before addition of TFA (150 μL, 1.947 mmol). The resulting mixture was left to stir at ambient temperature overnight. The reaction was heated to 35° C. and left to stir for a further 3 hours then left to stir at room temperature for a further 72 hours. The reaction mixture was concentrated in vacuo and azeotroped with MeOH to remove excess TFA. The resulting residue was purified by preparative reverse phase HPLC (basic eluent) to give rel-(2R*,3S*,4S*,5R*)-3-(3,4-difluoro-2-methoxyphenyl)-N-(7-hydroxy-6,7-dihydro-5H-cyclopenta[b]pyridin-3-yl)-4,5-dimethyl-5-(trifluoromethyl)tetrahydrofuran-2-carboxamide (17, 8.1 mg, 74%) as a white solid. ¹H NMR (500 MHz, DMSO-d₆) δ 10.32 (s, 1H), 8.54 (d, J=2.2 Hz, 1H), 7.89 (d, J=2.2 Hz, 1H), 7.20-7.11 (m, 2H), 5.25 (d, J=5.5 Hz, 1H), 5.08 (d, J=10.3 Hz, 1H), 4.89-4.84 (m, 1H), 4.23 (dd, J=10.3, 7.7 Hz, 1H), 3.94 (d, J=2.0 Hz, 3H), 2.94-2.86 (m, 1H), 2.79-2.66 (m, 2H), 2.37-2.28 (m, 1H), 1.85-1.77 (m, 1H), 1.60 (s, 3H), 0.73 (d, J=6.3 Hz, 3H) ppm. ESI-MS m/z calc. 486.1578, found 487.6 (M+1)⁺; 485.5 (M−1)⁻; Retention time: 3.25 minutes.

General Method N: Deprotection of Silyl Groups with HCl (e.g., 18)

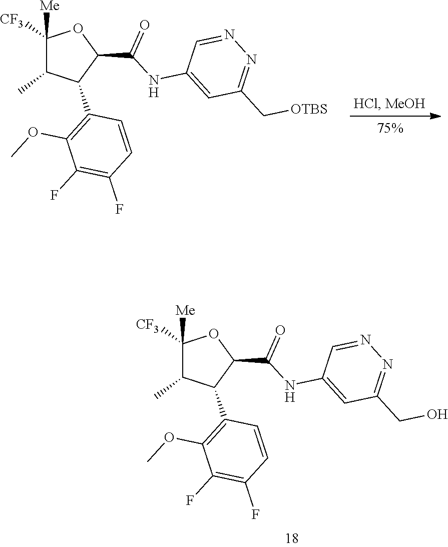 [SMILES: COc1c([C@H]2[C@H](C(=O)Nc3cnnc(CO[Si](C)(C)C(C)(C)C)c3)O[C@@](C)(C(F)(F)F)[C@H]2C)ccc(F)c1F ; COc1c([C@H]2[C@H](C(=O)Nc3cnnc(CO)c3)O[C@@](C)(C(F)(F)F)[C@H]2C)ccc(F)c1F]

HCl (60 μL of 37% w/v, 0.6089 mmol) was added to a solution of (2R,3S,4S,5R)—N-(6-(((tert-butyldimethylsilyl)oxy)methyl)pyridazin-4-yl)-3-(3,4-difluoro-2-methoxyphenyl)-4,5-dimethyl-5-(trifluoromethyl)tetrahydrofuran-2-carboxamide (40 mg, 0.06949 mmol) in MeOH (1 mL) and the reaction mixture was stirred at ambient temperature for 90 minutes. The mixture was concentrated in vacuo, and filtered through a sodium bicarbonate cartridge, washing with methanol. The filtrate was concentrated in vacuo to give (2R,3S,4S,5R)-3-(3,4-difluoro-2-methoxyphenyl)-N-(6-(hydroxymethyl)pyridazin-4-yl)-4,5-dimethyl-5-(trifluoromethyl)tetrahydrofuran-2-carboxamide (18, 24.6 mg, 75%) as a white solid. ¹H NMR (400 MHz, Chloroform-d) δ 10.68 (s, 1H), 9.53 (s, 1H), 8.57 (s, 1H), 7.12 (s, 1H), 6.95-6.83 (m, 1H), 5.20 (d, J=10.4 Hz, 1H), 5.00 (d, J=6.7 Hz, 2H), 4.25 (s, 1H), 4.03-3.96 (m, 3H), 2.75 (dt, J=13.6, 6.7 Hz, 1H), 1.71 (s, 3H), 0.79 (d, J=7.3 Hz, 3H) ppm. ESI-MS m/z calc. 461.1374, found 462.6 (M+1); 460.5 (M−1)⁻; Retention time: 3.0 minutes.

### General Method O: Ester Hydrolysis to Acid (e.g., 19)

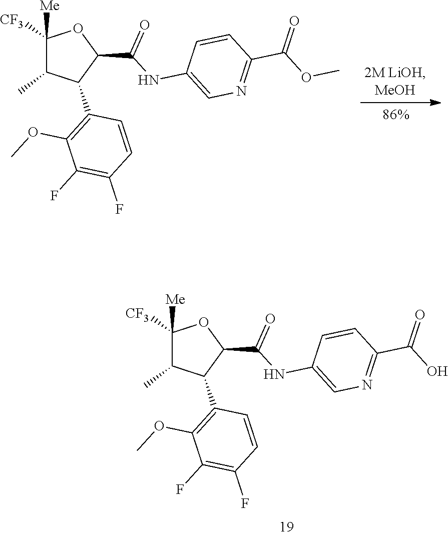

To a suspension of methyl 5-((2R,3S,4S,5R)-3-(3,4-difluoro-2-methoxyphenyl)-4,5-dimethyl-5-(trifluoromethyl)tetrahydrofuran-2-carboxamido)picolinate (1.54 g, 2.680 mmol) in MeOH (10 mL) was added LiOH aqueous solution (5.4 mL of 2 M, 10.80 mmol), followed by stirring at ambient temperature for 1.5 hours. The reaction was then acidified to pH 1 with 1 M aqueous HCl (20 mL) and water (10 mL) was added followed by extraction with EtOAc (3×30 mL). The combined organics were washed with brine (30 mL), dried (MgSO₄) and concentrated under reduced pressure to give 5-((2R,3S,4S,5R)-3-(3,4-difluoro-2-methoxyphenyl)-4,5-dimethyl-5-(trifluoromethyl)tetrahydrofuran-2-carboxamido)picolinic acid (1.12 g, 86%) as a pale yellow glass. For characterisation, a 50 mg sample of this material was repurified preparative reverse phase HPLC (basic eluent) to give 5-((2R,3S,4S,5R)-3-(3,4-difluoro-2-methoxyphenyl)-4,5-dimethyl-5-(trifluoromethyl)tetrahydrofuran-2-carboxamido)picolinic acid (19, 31 mg) as a white solid. ¹H NMR (500 MHz, DMSO-d₆) δ 10.69 (s, 1H), 8.82 (d, J=2.4 Hz, 1H), 8.15 (dd, J=8.6, 2.5 Hz, 1H), 7.95 (d, J=8.6 Hz, 1H), 7.16 (dd, J=8.6, 4.5 Hz, 2H), 5.14 (d, J=10.3 Hz, 1H), 4.26 (dd, J=10.3, 7.6 Hz, 1H), 3.95 (d, J=2.0 Hz, 3H), 2.77 (p, J=7.6 Hz, 1H), 1.61 (s, 3H), 0.73 (d, J=4.7 Hz, 3H) ppm. ESI-MS m/z calc. 474.1214, found 475.3 (M+1); 473.2 (M−1); Retention time: 2.5 minutes.

General Method P: Ester Reduction to Alcohol with LiAlH₄ (e.g., 20)

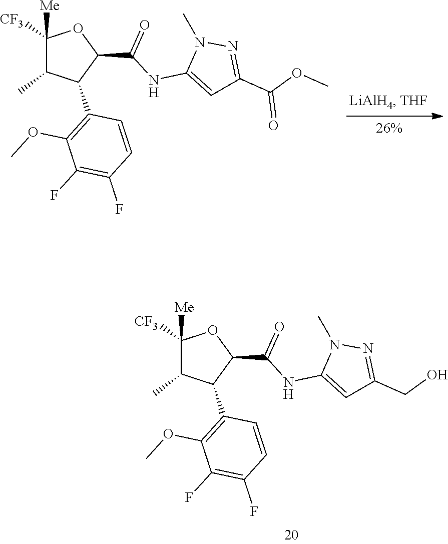

Methyl 5-((2R,3S,4S,5R)-3-(3,4-difluoro-2-methoxyphenyl)-4,5-dimethyl-5-(trifluoromethyl)tetrahydrofuran-2-carboxamido)-1-methyl-1H-pyrazole-3-carboxylate was dissolved in THF (3 mL) and treated with LiAlH₄ (in THF) (375 μL of 1 M, 0.3750 mmol). The mixture was stirred at room temperature under nitrogen. The reaction was quenched with MeOH and concentrated. The residue was purified by preparative reverse phase HPLC (basic eluent) to give (2R,3S,4S,5R)-3-(3,4-difluoro-2-methoxyphenyl)-N-(3-(hydroxymethyl)-1-methyl-1H-pyrazol-5-yl)-4,5-dimethyl-5-(trifluoromethyl)tetrahydrofuran-2-carboxamide (20, 37.2 mg, 26%) as a white solid. ¹H NMR (500 MHz, DMSO-d₆) δ 10.15 (s, 1H), 7.19 (dd, J=8.5, 5.2 Hz, 2H), 6.11 (s, 1H), 5.13 (d, J=10.4 Hz, 1H), 4.92 (t, J=5.8 Hz, 1H), 4.29 (d, J=5.7 Hz, 2H), 4.20 (dd, J=10.4, 7.6 Hz, 1H), 3.95 (d, J=2.0 Hz, 3H), 3.50 (s, 3H), 2.75 (t, J=7.5 Hz, 1H), 1.61 (s, 3H), 0.80-0.68 (m, 3H) ppm. ESI-MS m/z calc. 463.15305, found 464.3 (M+1)⁺; Retention time: 3.04 minutes.

### General Method Q: Cu Catalysed C—N Coupling (e.g., 21)

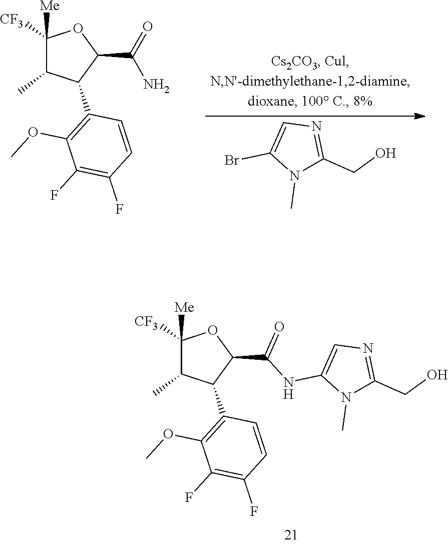

(2R,3S,4S,5R)-3-(3,4-difluoro-2-methoxy-phenyl)-4,5-dimethyl-5-(trifluoromethyl)tetrahydrofuran-2-carboxamide (60 mg, 0.1613 mmol), N,N-dimethylethane-1,2-diamine (10 μL, 0.09393 mmol), cesium carbonate (105 mg, 0.3223 mmol) and (5-bromo-1-methyl-imidazol-2-yl)methanol (42 mg, 0.2199 mmol) were suspended in dioxane (1 mL). The reaction mixture was degassed and purged with nitrogen before addition of CuI (9 mg, 0.04726 mmol). The vial was sealed and heated thermally at 100° C. for 18 hours and then at ambient temperature for 2 days. The mixture was filtered through a pad of celite, washing with EtOAc and concentrated in-vacuo. The material was purified by preparative reverse phase HPLC (basic eluent) to afford a yellow oil. The oil was taken up in MeOH and loaded on to SCX-2 (2 g) cartridge. The cartridge was flushed with MeOH (25 ml) and the product was then eluted with 2M NH₃ in MeOH (30 ml). The basic eluent was concentrated in vacuo and purified further by achiral SFC using a Chiralpak ID column, 5 μm particle size, 25 cm×20 mm from Daicel, to give (2R,3S,4S,5R)-3-(3,4-difluoro-2-methoxyphenyl)-N-(2-(hydroxymethyl)-1-methyl-1H-imidazol-5-yl)-4,5-dimethyl-5-(trifluoromethyl)tetrahydrofuran-2-carboxamide (21, 6 mg, 8%). ¹H NMR (400 MHz, DMSO-d₆) δ 9.90 (s, 1H), 7.30-7.11 (m, 2H), 6.66 (s, 1H), 5.21 (s, 1H), 5.10 (d, J=10.6 Hz, 1H), 4.40 (s, 2H), 4.18 (dd, J=10.5, 7.6 Hz, 1H), 3.94 (d, J=2.1 Hz, 3H), 3.28 (s, 3H), 2.74 (p, J=7.6 Hz, 1H), 1.61 (s, 3H), 0.74 (d, J=7.0 Hz, 3H) ppm. ESI-MS m/z calc. 463.15305, found 464.0 (M+1)⁺; 462.0 (M−1)⁻; Retention time: 2.91 minutes.

### General Method R: Benzyl Deprotection Via Hydrogenation (e.g., 22)

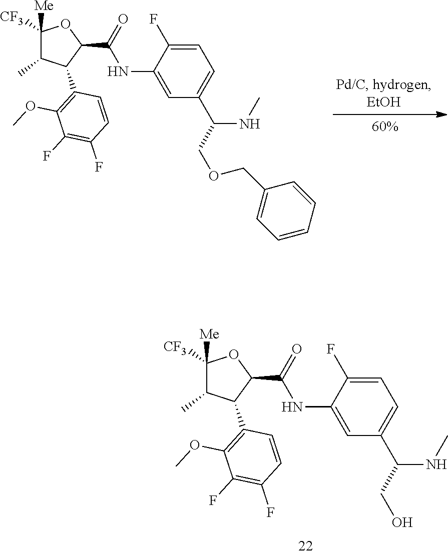

A solution of rel-(2R*,3S*,4S*,5R*)-N-(5-(2-(benzyloxy)-1-(methylamino)ethyl)-2-fluorophenyl)-3-(3,4-difluoro-2-methoxyphenyl)-4,5-dimethyl-5-(trifluoromethyl)tetrahydrofuran-2-carboxamide (20 mg, 0.0327 mmol) in EtOH (20 mL) was flushed with nitrogen three times using vacuum/nitrogen cycles. Pd/C (100 mg, 0.94 mmol) was added and the solution was placed under nitrogen again. The mixture was placed under a hydrogen balloon and stirred overnight. The mixture was then filtered through celite and concentrated. The crude product was purified by flash column chromatography (12 g SiO₂, eluting with 0 to 100% EtOAc in heptanes). Product fractions were combined and concentrated in vacuo to give rel-(2R*,3S*,4S*,5R*)-3-(3,4-difluoro-2-methoxyphenyl)-N-(5-fluoro-2-(2-hydroxy-1-(methylamino)ethyl)pyridin-4-yl)-4,5-dimethyl-5-(trifluoromethyl)tetrahydrofuran-2-carboxamide (22, 11 mg, 60%) as a white solid. ¹H NMR (500 MHz, DMSO-d₆) δ 10.46 (s, 1H), 8.64 (s, 1H), 8.28 (s, 1H), 7.19 (s, 2H), 5.38 (d, J=10.4 Hz, 1H), 5.35 (s, 1H), 4.31 (d, J=5.7 Hz, 1H), 4.27 (dd, J=10.4, 7.5 Hz, 1H), 3.96 (s, 3H), 3.72 (dq, J=28.6, 6.0, 5.5 Hz, 2H), 3.28 (s, 1H), 2.79 (p, J=7.2 Hz, 1H), 2.44 (s, 3H), 1.61 (s, 3H), 0.74 (d, J=7.4 Hz, 3H) ppm. ESI-MS m/z calc. 521.1749, found 523.4 (M+1)⁺; Retention time: 3.26 minutes.

General Method S: Alcohol Mesylation and Displacement with Amines (e.g., 23)

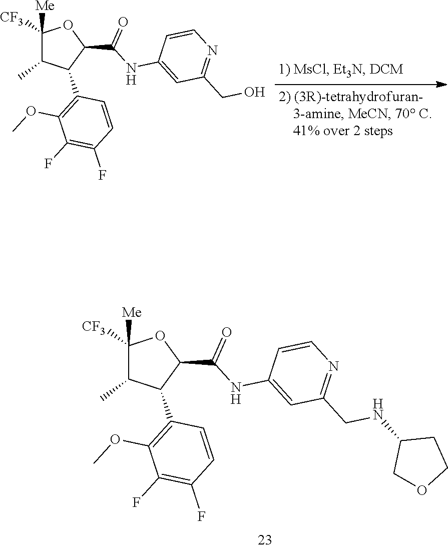 [SMILES: COc1c([C@H]2[C@H](C(=O)Nc3ccnc(CO)c3)O[C@@](C)(C(F)(F)F)[C@H]2C)ccc(F)c1F ; COc1c([C@H]2[C@H](C(=O)Nc3ccnc(CN[C@@H]4CCOC4)c3)O[C@@](C)(C(F)(F)F)[C@H]2C)ccc(F)c1F]

### Step 1:

(2R,3S,4S,5R)-3-(3,4-difluoro-2-methoxyphenyl)-N-(2-(hydroxymethyl)pyridin-4-yl)-4,5-dimethyl-5-(trifluoromethyl)tetrahydrofuran-2-carboxamide (14, 200 mg, 0.4344 mmol) in DCM (2 mL) was cooled in an ice bath under nitrogen before the addition of triethylamine (150 μL, 1.076 mmol) followed by methanesulfonyl chloride (50 μL, 0.6460 mmol). The reaction was concentrated to give [4-[[(2R,3S,4S,5R)-3-(3,4-difluoro-2-methoxy-phenyl)-4,5-dimethyl-5-(trifluoromethyl)tetrahydrofuran-2-carbonyl]amino]-2-pyridyl]methyl methanesulfonate (triethylamine salt) (277.9 mg, 100%) which was used without purification in the following step.

### Step 2:

To a solution of 4-[[(2R,3S,4S,5R)-3-(3,4-difluoro-2-methoxy-phenyl)-4,5-dimethyl-5-(trifluoromethyl)tetrahydrofuran-2-carbonyl]amino]-2-pyridyl]methyl methanesulfonate (triethylamine salt) (100 mg, 0.1563 mmol) in acetonitrile (0.5 mL) was added (3R)-tetrahydrofuran-3-amine (45 mg, 0.5165 mmol). The reaction mixture was sealed and heated at 70° C. for 5 hours. The reaction was then filtered and purified by preparative reverse phase HPLC (basic eluent) to give (2R,3S,4S,5R)-3-(3,4-difluoro-2-methoxyphenyl)-4,5-dimethyl-N-(2-((((R)-tetrahydrofuran-3-yl)amino)methyl)pyridin-4-yl)-5-(trifluoromethyl)tetrahydrofuran-2-carboxamide (23, 34.2 mg, 41%). ¹H NMR (500 MHz, DMSO-d₆) δ 10.55 (s, 1H), 8.35 (d, J=5.5 Hz, 1H), 7.64 (d, J=2.0 Hz, 1H), 7.51 (dd, J=5.6, 2.1 Hz, 1H), 7.30-7.06 (m, 2H), 5.09 (d, J=10.3 Hz, 1H), 4.25 (dd, J=10.3, 7.6 Hz, 1H), 3.96 (d, J=2.0 Hz, 3H), 3.82-3.58 (m, 5H), 3.44 (dd, J=8.6, 4.2 Hz, 1H), 3.29 (dd, J=5.2, 1.7 Hz, 1H), 2.78 (p, J=7.5 Hz, 1H), 2.39 (d, J=22.7 Hz, 1H), 1.93 (dq, J=12.5, 7.2 Hz, 1H), 1.74-1.63 (m, 1H), 1.60 (s, 3H), 0.73 (dd, J=7.3, 2.4 Hz, 3H) ppm. ESI-MS m/z calc. 529.2, found 530.3 (M+1)⁺; Retention time: 3.2 minutes.

General Method T: Vinyl Epoxidation and Ring Opening with Nucleophiles (e.g., 24)

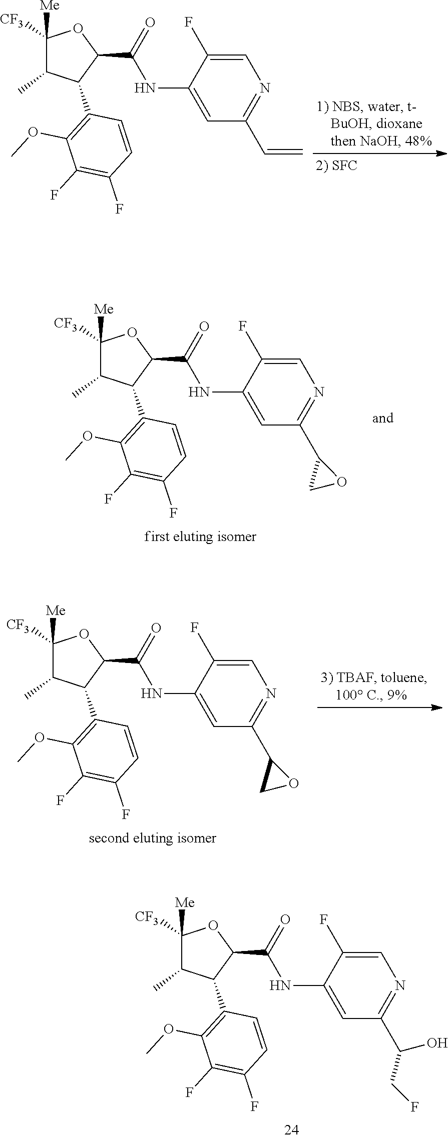

### Step 1:

NBS (860 mg, 4.832 mmol) was added to a stirred suspension of (2R,3S,4S,5R)-3-(3,4-difluoro-2-methoxy-phenyl)-N-(5-fluoro-2-vinyl-4-pyridyl)-4,5-dimethyl-5-(trifluoromethyl)tetrahydrofuran-2-carboxamide (2.21 g, 4.659 mmol) in water (30 mL) and t-BuOH (15 mL). After the addition was complete, the reaction was heated to 45° C. for 1 hour. Dioxane (10 mL) was added to the mixture (to aid solubility). The reaction mixture was then left to stir at 45° C. for a further 1 hour. The reaction mixture was cooled to 0° C., NaOH (560 mg, 14.00 mmol) in water (9 mL) was added dropwise and the mixture stirred for a further 10 minutes at 0° C. The reaction was then diluted with EtOAc (50 mL) and poured over water (50 mL). The organic layer was separated, and the aqueous layer was extracted with EtOAc (2×50 mL). The organic layers were combined, washed with brine (10 mL), dried (Na₂SO₄) and concentrated in vacuo. The crude material was purified by flash column chromatography (80 g SiO₂, eluting with 0 to 60% EtOAc in heptane) to give (2R,3S,4S,5R)-3-(3,4-difluoro-2-methoxyphenyl)-N-(5-fluoro-2-(oxiran-2-yl)pyridin-4-yl)-4,5-dimethyl-5-(trifluoromethyl)tetrahydrofuran-2-carboxamide (1.086 g, 48%) as off-white solid as a mixture of epimers at the oxiranyl position. ¹H NMR (500 MHz, Chloroform-d) δ 8.84 (s, 1H), 8.40-8.39 (m, 1H), 8.24 (dd, J=8.1, 6.1 Hz, 1H), 7.11-7.06 (m, 1H), 6.95-6.88 (m, 1H), 5.04 (dd, J=11.1, 6.4 Hz, 1H), 4.11-4.06 (m, 1H), 4.01 (d, J=2.9 Hz, 3H), 3.92-3.89 (m, 1H), 3.10 (ddd, J=5.8, 4.1, 3.0 Hz, 1H), 2.93 (ddd, J=9.9, 5.8, 2.5 Hz, 1H), 2.80-2.72 (m, 1H), 1.68 (s, 3H), 0.81-0.77 (m, 3H) ppm. ESI-MS m/z calc. 490.13272, found 490.7 (M+1)⁺; 489.0 (M−1)⁻; Retention time: 4.24 minutes.

### Step 2:

SFC separation of (2R*,3S*,4S*,5R*)-3-(3,4-difluoro-2-methoxyphenyl)-N-(5-fluoro-2-(oxiran-2-yl)pyridin-4-yl)-4,5-dimethyl-5-(trifluoromethyl)tetrahydrofuran-2-carboxamide (1.00 g, 2.039 mmol) using a Chiralpak IG column, column, 5 μm particle size, 25 cm×20 mm from Daicel gave:

First eluting isomer (rt=4.06 minutes): rel-(2R*,3S*,4S*,5R*)-3-(3,4-difluoro-2-methoxyphenyl)-N-(5-fluoro-2-(oxiran-2-yl)pyridin-4-yl)-4,5-dimethyl-5-(trifluoromethyl)tetrahydrofuran-2-carboxamide (348 mg, 33%). ESI-MS m/z calc. 490.13272, found 490.6 (M+1)⁺; 488.9 (M−1)⁻; Retention time: 3.54 minutes.

Second eluting isomer (rt=5.04 minutes): rel-(2R*,3S*,4S*,5R*)-3-(3,4-difluoro-2-methoxyphenyl)-N-(5-fluoro-2-(oxiran-2-yl)pyridin-4-yl)-4,5-dimethyl-5-(trifluoromethyl)tetrahydrofuran-2-carboxamide (377 mg, 35%). ESI-MS m/z calc. 490.13272, found 490.7 (M+1)⁺; 488.9 (M−1)⁻; Retention time: 3.54 minutes.

### Step 3:

rel-(2R*,3S*,4S*,5R*)-3-(3,4-difluoro-2-methoxyphenyl)-N-(5-fluoro-2-(oxiran-2-yl)pyridin-4-yl)-4,5-dimethyl-5-(trifluoromethyl)tetrahydrofuran-2-carboxamide (first eluting isomer by SFC, 50 mg, 0.09992 mmol) was dissolved in toluene (2.0 mL) and TBAF (in THF) (1.0 mL of 1 M, 1.000 mmol) was added. The resulting mixture was left to stir at 80° C. for 1 hour and then 100° C. overnight. The reaction mixture was concentrated in vacuo and purified by flash column chromatography (12 g SiO₂, eluting with 0 to 100% EtOAc in heptane). The mixture was further purified by preparative reverse phase HPLC (basic eluent) to give rel-(2R*,3S*,4S*,5R*)-3-(3,4-difluoro-2-methoxyphenyl)-N-(5-fluoro-2-(2-fluoro-1-hydroxyethyl)pyridin-4-yl)-4,5-dimethyl-5-(trifluoromethyl)tetrahydrofuran-2-carboxamide (24, 4.9 mg, 9%) as white solid. ¹H NMR (500 MHz, DMSO-d₆) δ 10.27 (s, 1H), 8.49 (d, J=2.3 Hz, 1H), 8.27 (d, J=6.4 Hz, 1H), 7.22-7.15 (m, 2H), 5.95 (d, J=5.0 Hz, 1H), 5.34 (d, J=10.4 Hz, 1H), 4.82-4.74 (m, 1H), 4.62 (ddd, J=47.7, 9.5, 3.1 Hz, 1H), 4.48 (ddd, J=47.7, 9.5, 6.0 Hz, 1H), 4.25 (dd, J=10.4, 7.6 Hz, 1H), 3.95 (s, 3H), 2.81-2.73 (m, 1H), 1.61 (s, 3H), 0.73 (d, J=5.2 Hz, 3H) ppm. ESI-MS m/z calc. 510.13895, found 510.9 (M+1)⁺; 509.0 (M−1)⁻; Retention time: 3.42 minutes.

### Example 1

rel-(2R,3S,4S,5R)-3-(3,4-difluoro-2-methoxyphenyl)-4,5-dimethyl-N-(pyridazin-4-yl)-5-(trifluoromethyl)tetrahydrofuran-2-carboxamide (25) and rel-(2S,3R,4R,5S)-3-(3,4-difluoro-2-methoxyphenyl)-4,5-dimethyl-N-(pyridazin-4-yl)-5-(trifluoromethyl)tetrahydrofuran-2-carboxamide (26)

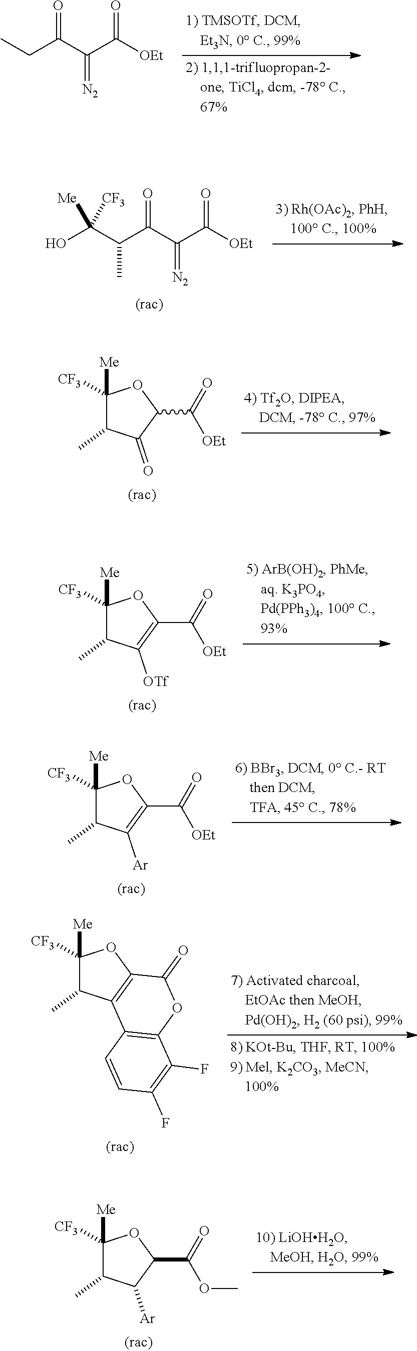

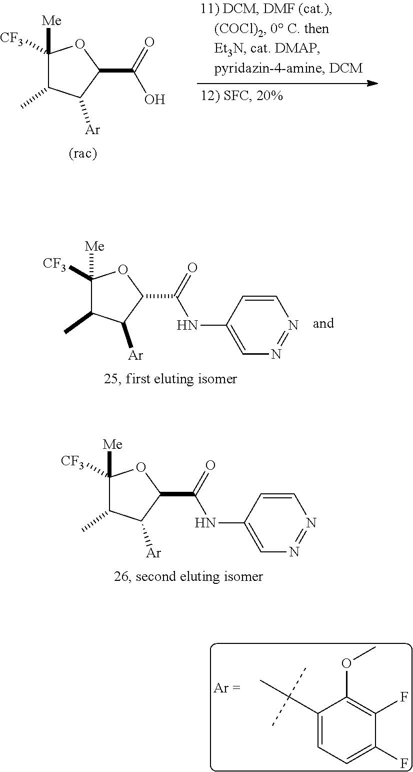

### Step 1:

NEt₃ (7.7 mL, 55.2 mmol) was added to a solution of ethyl 2-diazo-3-oxo-pentanoate (6.69 g, 39.3 mmol) in DCM (80 mL) with stirring at 0° C. under nitrogen. Trimethylsilyl trifluoromethanesulfonate (8.5 mL, 47.0 mmol) was added dropwise over 5 mins and the mixture was stirred for a further 30 mins at 0° C. The reaction mixture was diluted with pentane (100 mL), the layers separated and the organic phase washed with dilute aqueous sodium bicarbonate (100 mL) and brine (100 mL). The organic layer was dried (MgSO₄), and concentrated in vacuo to give ethyl (Z)-2-diazo-3-trimethylsilyloxy-pent-3-enoate (9.4 g, 99%) as a red oil. ¹H NMR (500 MHz, Chloroform-d) δ 5.33 (q, J=7.0 Hz, 1H), 4.25 (q, J=7.1 Hz, 2H), 1.67 (d, J=7.0 Hz, 3H), 1.29 (t, J=7.1 Hz, 3H), 0.22 (s, 9H) ppm.

### Step 2:

To a solution of 1,1,1-trifluoropropan-2-one (8 mL, 89.4 mmol) in DCM (80 mL) stirring at -78° C. was added TiCl₄ (70 mL of 1 M in DCM, 70.00 mmol) via cannula. To the resulting solution, a solution of ethyl (Z)-2-diazo-3-trimethylsilyloxy-pent-3-enoate (36.1 g of 31.3% w/w, 46.6 mmol) in 40 mL of DCM was added dropwise over 15 mins. After 100 mins the reaction was carefully quenched with water, allowing the temperature to rise slowly, and then extracted with DCM. The combined organic layers were dried (MgSO₄), filtered, and concentrated in vacuo. Purification by flash chromatography (330 g SiO₂, 0 to 20% EtOAc in heptane) gave ethyl 2-diazo-6,6,6-trifluoro-5-hydroxy-4,5-dimethyl-3-oxo-hexanoate (8.82 g, 67%), which was stored as a solution in toluene. ¹H NMR (500 MHz, Chloroform-d) δ 4.33 (q, J=7.1 Hz, 2H), 4.14 (q, J=7.0 Hz, 1H), 3.98 (s, 1H), 1.43 (q, J=1.2 Hz, 3H), 1.35 (t, J=7.1 Hz, 3H), 1.31 (dq, J=7.0, 1.4 Hz, 3H) ppm. ESI-MS m/z calc. 282.08273, found 283.1 (M+1)⁺; 281.0 (M−1)⁻.

### Step 3:

A solution of rhodium tetraacetate (245 mg, 0.55 mmol) in benzene (32 mL) was heated at reflux for 10 min before a solution of ethyl 2-diazo-6,6,6-trifluoro-5-hydroxy-4,5-dimethyl-3-oxo-hexanoate (10 g, 35.4 mmol) in benzene (13 mL) was added slowly via addition funnel while refluxing for 60 mins. The mixture was then concentrated in vacuo to give ethyl rac-(4R,5R)-4,5-dimethyl-3-oxo-5-(trifluoromethyl)tetrahydrofuran-2-carboxylate (9.0 g, 100%) as a green coloured residue containing residual catalyst, and as a mixture of epimers at the position next to the ester. This material was used without further purification. ¹H NMR (500 MHz, Chloroform-d) δ 4.83-4.57 (m, 1H), 4.38-4.16 (m, 2H), 2.60 (dddd, J=9.3, 8.2, 5.6, 1.4 Hz, 1H), 1.73-1.63 (m, 3H), 1.30 (t, J=7.1 Hz, 3H), 1.24 (ddq, J=6.4, 4.1, 1.9 Hz, 3H) ppm.

### Step 4:

To a stirred solution of ethyl rac-(4R,5R)-4,5-dimethyl-3-oxo-5-(trifluoromethyl)tetrahydrofuran-2-carboxylate (48 g, 188.83 mmol) in DCM (400 mL) stirring at −78° C. was added DIPEA (29.680 g, 40 mL, 229.64 mmol). A solution of trifluoromethylsulfonyl trifluoromethanesulfonate (53.440 g, 32 mL, 189.41 mmol) in DCM (200 mL) was added to the reaction mixture at the same temperature over 1 h. The reaction mixture was stirred for 30 mins at 0° C. before being quenched with 100 mL saturated aqueous NaHCO₃ solution. The organic layer was separated and aqueous layer extracted with DCM (160 mL). The combined organic layers were dried (MgSO₄) and concentrated in vacuo to give ethyl rac-(4R,5R)-2,3-dimethyl-2-(trifluoromethyl)-4-(trifluoromethylsulfonyloxy)-3H-furan-5-carboxylate (71 g, 97%). ¹H NMR (400 MHz, Chloroform-d) δ 4.38-4.32 (m, 2H), 3.29-3.23 (m, 1H), 1.64 (s, 3H), 1.37-1.33 (m, 6H) ppm.

### Step 5:

To stirred a solution of ethyl rac-(4R,5R)-2,3-dimethyl-2-(trifluoromethyl)-4-(trifluoromethylsulfonyloxy)-3H-furan-5-carboxylate (26 g, 67.311 mmol) in toluene (130.00 mL) was added (3,4-difluoro-2-methoxy-phenyl)boronic acid (14 g, 74.5 mmol) followed by K₃PO₄ (100 mL of 2 M, 200.00 mmol) under an argon atmosphere. The reaction was degassed before tetrakis(triphenylphosphine)palladium(0) (4 g, 3.46 mmol) was added. After further degassing, the reaction was heated at 100° C. for 2 hours. The reaction was diluted in water and the aqueous layer extracted with EtOAc (2×100 mL). The combined organic layers were concentrated in vacuo. Purification by flash chromatography (SiO₂, 0 to 10% EtOAc in heptane) gave ethyl 4-(3,4-difluoro-2-methoxy-phenyl)-2,3-dimethyl-2-(trifluoromethyl)-3H-furan-5-carboxylate (24.4 g, 93%) as a 6:1 diastereomeric mixture, with the major isomer believed to be ethyl rac-(4R,5R)-4-(3,4-difluoro-2-methoxy-phenyl)-2,3-dimethyl-2-(trifluoromethyl)-3H-furan-5-carboxylate. Major isomer: ¹H NMR (400 MHz, Chloroform-d) δ 6.88-6.79 (m, 2H), 4.17-4.09 (m, 2H), 3.90 (s, 3H), 3.46 (q, J=7.4 Hz, 1H), 1.67 (s, 3H), 1.12 (t, J=7.4 Hz, 3H), 1.06 (dd, J=5.4, 2.7 Hz, 3H) ppm. Minor isomer ¹H NMR (400 MHz, Chloroform-d) δ 6.88-6.79 (m, 2H), 4.17-4.09 (m, 2H), 3.88 (s, 3H), 3.76-3.71 (m, 1H), 1.51 (s, 3H), 1.12 (t, J=7.4 Hz, 3H), 0.99 (dd, J=5.4, 2.7 Hz, 3H) ppm. ESI-MS m/z calc. 380.1047, found 381.02 (M+1).

### Step 6:

To an ice-cooled solution of ethyl 4-(3,4-difluoro-2-methoxy-phenyl)-2,3-dimethyl-2-(trifluoromethyl)-3H-furan-5-carboxylate (110 g, 243.0 mmol) in DCM (360 mL) was added BBr₃ (370 mL of 1 M, 370.0 mmol) dropwise. Upon completion the mixture was quenched by addition of water and aqueous sodium bicarbonate solution, the aqueous layer extracted with DCM and the combined organic layers dried (MgSO₄) and concentrated in vacuo. The residue was dissolved in DCM (430 mL) at ambient temperature and TFA (40 mL, 519.2 mmol) was added, then the reaction was heated to 45° C. Upon completion, the mixture was quenched by addition of aqueous sodium bicarbonate solution and the aqueous layer extracted with DCM, dried (MgSO₄) and concentrated in vacuo to give the desired product in a 5:1 mixture of diastereomers. Recrystallization was carried out by solubilizing the crude in the smallest possible amount of DCM and adding a layer of heptane on top of this solution (liquid-liquid diffusion). After approx. 1 hour, 56.5 g (d.r. 97:3 syn:anti) from the first and second crystallization was obtained, and a further 4.6 g (d.r. 96:4 syn:anti) from the third crystallization was obtained. The first to third batches were combined to give 6,7-difluoro-1,2-dimethyl-2-(trifluoromethyl)-1H-furo[2,3-c]chromen-4-one (61 g, 78%), with the major isomer believed to be rac-(1S,2R)-6,7-difluoro-1,2-dimethyl-2-(trifluoromethyl)-1H-furo[2,3-c]chromen-4-one. ESI-MS m/z calc. 320.04718, found 321.5 (M+1)⁺; 319.6 (M−1)⁻.

### Step 7:

rac-(1S,2R)-6,7-Difluoro-1,2-dimethyl-2-(trifluoromethyl)-1H-furo[2,3-c]chromen-4-one (30 g, 93.69 mmol) was dissolved in EtOAc (400 mL) and stirred with activated charcoal (6 g, 499.6 mmol) (0.2 g/g of substrate) at ambient temperature for 4 hours and 30 minutes. The mixture was filtered through a pad of celite, washing with EtOAc. The filtrate was concentrated in vacuo to give a white solid. The white solid was suspended in MeOH (600 mL) and added to a suspension of Pd(OH)₂ (13.62 g of 20% w/w, 19.40 mmol) in MeOH (150 mL) in a 2.25 L Parr bottle. The resulting mixture was shaken in the Parr hydrogenator under a hydrogen pressure of 60 psi overnight. The suspension was filtered through celite under a nitrogen atmosphere, rinsed with MeOH and then with EtOAc, and the resulting filtrate was concentrated in vacuo to give methyl rac-(2R,3S,4S,5R)-3-(3,4-difluoro-2-hydroxy-phenyl)-4,5-dimethyl-5-(trifluoromethyl)tetrahydrofuran-2-carboxylate (32.75 g, 99%). ¹H NMR (400 MHz, Methanol-d4) δ 7.05 (ddq, J=9.4, 5.9, 1.9 Hz, 1H), 6.57 (ddd, J=10.0, 9.0, 7.6 Hz, 1H), 5.01 (d, J=6.0 Hz, 1H), 4.34 (dd, J=8.4, 6.0 Hz, 1H), 3.49 (s, 3H), 3.01-2.86 (m, 1H), 1.50 (q, J=1.2 Hz, 3H), 0.89 (dq, J=7.6, 1.9 Hz, 3H) ppm. ESI-MS m/z calc. 354.08905, found 353.3 (M−1)⁻.

### Step 8:

A solution of methyl rac-(2R,3S,4S,5R)-3-(3,4-difluoro-2-hydroxy-phenyl)-4,5-dimethyl-5-(trifluoromethyl)tetrahydrofuran-2-carboxylate (60.8 g, 171.6 mmol) in THF (620 mL) was cooled to 1° C., and potassium tert-butoxide (65.0472 g, 579.7 mmol) was added over 10 mins, keeping the internal temperature below 10° C. The mixture was stirred at 0° C. for a further 5 min, and then the mixture was warmed slightly. When the temperature had reached 13° C., the reaction was cooled down again with an ice bath before adding 2 M HCl (365 mL, to pH 1), keeping the internal temperature below 15° C. Water (300 mL) was added, the layers were separated, and the aqueous layer was extracted with EtOAc (110 mL). The combined organic extracts were washed with brine (300 mL), dried (MgSO₄), filtered and concentrated in vacuo to give rac-(2R,3S,4S,5R)-3-(3,4-difluoro-2-hydroxy-phenyl)-4,5-dimethyl-5-(trifluoromethyl)tetrahydrofuran-2-carboxylic acid (58.22 g, 100%). ¹H NMR (400 MHz, Methanol-d₄) δ 7.00 (ddd, J=8.4, 5.6, 2.3 Hz, 1H), 6.69 (ddd, J=10.1, 8.8, 7.5 Hz, 1H), 4.98 (d, J=10.5 Hz, 1H), 4.18 (dd, J=10.5, 7.6 Hz, 1H), 2.83 (p, J=7.5 Hz, 1H), 1.59 (q, J=1.2 Hz, 3H), 0.76 (dq, J=7.2, 2.2 Hz, 3H) ppm. ESI-MS m/z calc. 340.0734, found 339.0 (M−1)⁻.

### Step 9:

To a solution of rac-(2R,3S,4S,5R)-3-(3,4-difluoro-2-hydroxy-phenyl)-4,5-dimethyl-5-(trifluoromethyl)tetrahydrofuran-2-carboxylic acid (58.39 g, 171.6 mmol) in acetonitrile (300 mL) was added K₂CO₃ (82.6 g, 597.7 mmol) and MeI (37 mL, 594.3 mmol). The reaction was heated to 80° C. (internally temperature reached 61° C.) for 5 hours before being cooled to ambient temperature and diluted with DCM (350 mL). The mixture was filtered, washing the filter cake with more DCM (350 mL) and the filtrate was concentrated in vacuo to give methyl rac-(2R,3S,4S,5R)-3-(3,4-difluoro-2-methoxy-phenyl)-4,5-dimethyl-5-(trifluoromethyl)tetrahydrofuran-2-carboxylate (64.7 g, 100%) as an orange oil containing some residual K₂CO₃. This material was used in the next step without further purification. ¹H NMR (400 MHz, Chloroform-d) δ 6.91 (ddd, J=7.6, 5.7, 1.9 Hz, 1H), 6.85 (td, J=9.1, 7.2 Hz, 1H), 4.91 (d, J=10.2 Hz, 1H), 4.13 (dd, J=10.2, 8.0 Hz, 1H), 4.00 (d, J=2.7 Hz, 3H), 3.71 (s, 3H), 2.72 (p, J=7.7 Hz, 1H), 1.62 (q, J=1.2 Hz, 3H), 0.76 (dq, J=7.5, 2.4 Hz, 3H) ppm.

### Step 10:

Methyl rac-(2R,3S,4S,5R)-3-(3,4-difluoro-2-methoxy-phenyl)-4,5-dimethyl-5-(trifluoromethyl)tetrahydrofuran-2-carboxylate (63.2 g, 171.6 mmol) was dissolved in MeOH (500 mL) and water (300 mL). LiOH—H₂O (14.8882 g, 354.8 mmol) was added and the resultant mixture stirred at ambient temperature for 2 hours. The MeOH was removed in vacuo and the mixture was diluted in MTBE (320 mL). 2 M HCl (440 mL) was added to reach pH 1, the layers were separated and the aqueous layer extracted twice with MTBE (100 mL). The combined organic layers were dried (MgSO₄), filtered and concentrated in vacuo to give rac-(2R,3S,4S,5R)-3-(3,4-difluoro-2-methoxy-phenyl)-4,5-dimethyl-5-(trifluoromethyl)tetrahydrofuran-2-carboxylic acid (60.3 g, 99%) as an orange oil. ¹H NMR (400 MHz, DMSO-d₆) δ 12.96 (s, 1H), 7.40-6.82 (m, 2H), 4.96 (dd, J=15.5, 10.5 Hz, 1H), 4.08 (dd, J=10.4, 7.6 Hz, 1H), 3.93 (d, J=2.2 Hz, 3H), 2.67 (p, J=7.7 Hz, 1H), 1.59-1.49 (m, 3H), 0.77-0.63 (m, 3H) ppm. ESI-MS m/z calc. 354.08905, found 353.1 (M−1)⁻.

### Step 11:

To a solution of rac-(2R,3S,4S,5R)-3-(3,4-difluoro-2-methoxy-phenyl)-4,5-dimethyl-5-(trifluoromethyl)tetrahydrofuran-2-carboxylic acid (100 mg, 0.2823 mmol) in DCM (5 mL), stirring at 0° C. under nitrogen, was added DMF (2.2 μL, 0.02841 mmol) and carefully oxalyl chloride (75 μL, 0.8598 mmol). Gas evolution was observed. The reaction was warmed to ambient temperature and stirred for 15 minutes before being evaporated in vacuo. The residue was dissolved in DCM (3 mL) and added dropwise over 5 mins to a solution of pyridazin-4-amine (40 mg, 0.4206 mmol), DMAP (1.75 mg, 0.01432 mmol) and NEt₃ (120 μL, 0.8610 mmol) in DCM (5 mL) at 0° C. The reaction was allowed to warm to ambient and stirred overnight. The reaction mixture was diluted with DCM (50 mL) and washed with 2 M HCl solution (50 mL), dried using a phase separation cartridge and concentrated in vacuo. The material was then purified by preparative reverse phase HPLC (basic eluent) to afford of rac-(2R,3S,4S,5R)-3-(3,4-difluoro-2-methoxyphenyl)-4,5-dimethyl-N-(pyridazin-4-yl)-5-(trifluoromethyl)tetrahydrofuran-2-carboxamide. ESI-MS m/z calc. 431.12683, found 432.7 (M+1)⁺; 430.8 (M−1)⁻; Retention time: 3.15 minutes.

### Step 12:

The enantiomers of rac-(2R,3S,4S,5R)-3-(3,4-difluoro-2-methoxyphenyl)-4,5-dimethyl-N-(pyridazin-4-yl)-5-(trifluoromethyl)tetrahydrofuran-2-carboxamide were separated by chiral SFC (using a (R′R) Whelk 0-1 column, 3-5 μm particle size, 5.0 cm×3.0 mm from Regis Technologies with Solvent A: liquid CO₂ [58-60 bar/40° C.; Solvent B: methanol HPLC grade with 20 mM NH₃ on a UPC2-SFC instrument from Waters Corp.) to give:

First Eluting Isomer (r.t.=3.25 minutes): rel-(2S,3R,4R,5S)-3-(3,4-difluoro-2-methoxyphenyl)-4,5-dimethyl-N-(pyridazin-4-yl)-5-(trifluoromethyl)tetrahydrofuran-2-carboxamide (25, 6.1 mg, 10%) ESI-MS m/z calc. 431.12683, found 432.7 (M+1)⁺; 430.8 (M−1)⁻; Retention time: 3.15 minutes.

Second Eluting Isomer (r.t.=7.15 minutes): rel-(2R,3S,4S,5R)-3-(3,4-difluoro-2-methoxyphenyl)-4,5-dimethyl-N-(pyridazin-4-yl)-5-(trifluoromethyl)tetrahydrofuran-2-carboxamide (26, 6.5 mg, 10%). ¹H NMR (500 MHz, Chloroform-d) δ 9.08 (s, 1H), 8.90 (s, 1H), 8.59 (s, 1H), 7.92 (s, 1H), 6.90 (d, J=7.9 Hz, 1H), 6.79-6.70 (m, 1H), 4.90 (d, J=10.4 Hz, 1H), 3.96 (t, J=9.2 Hz, 1H), 3.85 (d, J=2.7 Hz, 3H), 2.64-2.57 (m, 1H), 1.82 (s, 3H), 0.67-0.61 (m, 3H) ppm. ESI-MS m/z calc. 431.12683, found 432.7 (M+1)⁺; 430.8 (M−1)⁻; Retention time: 3.15 minutes

The following compounds were made using a similar method to that described in Example 1, except that different coupling partners were used in the amide coupling step 11. In the Table below, “MS r.t.” stands for Mass Spec retention time.

| Cmpd | | LC/MS | Found | MS | |
|---|---|---|---|---|---|
| No | Compound Name | (m/z calc.) | M + 1 | r.t. | NMR (shifts in ppm) |
| 27 | rel-(2S,3R,4R,5S)-3- | 460.394 | 461.2 | 3.09 | ¹H NMR (500 MHZ, |
| | (3,4-difluoro-2- | | | | Chloroform-d) δ 8.31 (s, |
| | methoxyphenyl)-4,5- | | | | 1H), 7.21 (d, J = 7.4 Hz, |
| | dimethyl-N-(1-methyl- | | | | 1H), 7.05 (ddd, J = 8.1, 5.5, |
| | 2-oxo-1,2- | | | | 2.1 Hz, 1H), 6.89 (td, J = |
| | dihydropyridin-4-yl)- | | | | 9.2, 7.5 Hz, 1H), 6.74 (dd, |
| | 5-(trifluoromethyl) | | | | J = 7.5, 2.4 Hz, 1H), 6.51 (d, |
| | tetrahydrofuran-2- | | | | J = 2.4 Hz, 1H), 4.96 (d, J = |
| | carboxamide | | | | 10.8 Hz, 1H), 4.07 (dd, J = |
| | (first eluting isomer by | | | | 10.8, 8.1 Hz, 1H), 4.00 (d, |
| | SFC using Whelk-O1 | | | | J = 2.7 Hz, 3H), 3.49 (s, 3H), |
| | column) | | | | 2.73 (p, J = 7.7 Hz, 1H), |
| | | | | | 1.65 (s, 3H), 0.77 (dd, J = |
| | | | | | 7.6, 2.3 Hz, 3H) ppm. |
| 28 | rel-(2R,3S,4S,5R)-3- | 460.394 | 461.1 | 3.09 | ¹H NMR (500 MHZ, |
| | (3,4-difluoro-2- | | | | Chloroform-d) δ 8.32 (s, |
| | methoxyphenyl)-4,5- | | | | 1H), 7.21 (d, J = 7.5 Hz, |
| | dimethyl-N-(1-methyl- | | | | 1H), 7.04 (ddd, J = 8.1, 5.5, |
| | 2-oxo-1,2- | | | | 2.0 Hz, 1H), 6.88 (td, J = |
| | dihydropyridin-4-yl)- | | | | 9.2, 7.4 Hz, 1H), 6.74 (dd, |
| | 5- | | | | J = 7.5, 2.4 Hz, 1H), 6.51 (d, |
| | (trifluoromethyl) | | | | J = 2.3 Hz, 1H), 4.95 (d, J = |
| | tetrahydrofuran-2- | | | | 10.8 Hz, 1H), 4.07 (dd, J = |
| | carboxamide | | | | 10.8, 8.1 Hz, 1H), 3.99 (d, |
| | (second eluting isomer | | | | J = 2.6 Hz, 3H), 3.49 (s, 3H), |
| | by SFC using Whelk- | | | | 2.73 (p, J = 7.7 Hz, 1H), |
| | O1 column) | | | | 1.65 (s, 3H), 0.81-0.69 (m, |
| | | | | | 3H) ppm. |
| 29 | rel-(2S,3R,4R,5S)-3- | 430.368 | 431.2 | 3.24 | ¹H NMR (500 MHZ, |
| | (3,4-difluoro-2- | | | | DMSO-d₆) δ 10.40 (s, 1H), |
| | methoxyphenyl)-4,5- | | | | 8.76 (d, J = 2.3 Hz, 1H), |
| | dimethyl-N-(pyridin- | | | | 8.30 (dd, J = 4.7, 1.5 Hz, |
| | 3-yl)-5- | | | | 1H), 8.04 (ddd, J = 8.3, 2.6, |
| | (trifluoromethyl) | | | | 1.5 Hz, 1H), 7.35 (ddd, J = |
| | tetrahydrofuran-2- | | | | 8.3, 4.7, 0.8 Hz, 1H), 7.20- |
| | carboxamide | | | | 7.14 (m, 2H), 5.10 (d, J = |
| | (first eluting isomer by | | | | 10.3 Hz, 1H), 4.26 (dd, J = |
| | SFC using Whelk-O1 | | | | 10.3, 7.6 Hz, 1H), 3.96 (d, |
| | column) | | | | J = 2.1 Hz, 3H), 2.77 (p, J = |
| | | | | | 7.5 Hz, 1H), 1.61 (s, 3H), |
| | | | | | 0.75-0.73 (m, 3H) ppm. |
| 30 | rel-(2R,3S,4S,5R)-3- | 430.368 | 431.2 | 3.24 | ¹H NMR (500 MHZ, |
| | (3,4-difluoro-2- | | | | DMSO-d₆) δ 10.40 (s, 1H), |
| | methoxyphenyl)-4,5- | | | | 8.76 (dd, J = 2.5, 0.7 Hz, |
| | dimethyl-N-(pyridin- | | | | 1H), 8.30 (dd, J = 4.7, 1.5 |
| | 3-yl)-5- | | | | Hz, 1H), 8.04 (ddd, J = 8.3, |
| | (trifluoromethyl) | | | | 2.6, 1.5 Hz, 1H), 7.35 (ddd, |
| | tetrahydrofuran-2- | | | | J = 8.3, 4.7, 0.7 Hz, 1H), |
| | carboxamide | | | | 7.20-7.14 (m, 2H), 5.10 |
| | (second eluting isomer | | | | (d, J = 10.3 Hz, 1H), 4.26 |
| | by SFC using Whelk- | | | | (dd, J = 10.3, 7.7 Hz, 1H), |
| | O1 column) | | | | 3.96 (d, J = 2.1 Hz, 3H), |
| | | | | | 2.77 (p, J = 7.5 Hz, 1H), |
| | | | | | 1.61 (s, 3H), 0.75-0.72 (m, |
| | | | | | 3H) ppm. |
| 31 | rel-(2S,3R,4R,5S)-3- | 471.381 | 472.1 | 3.72 | |
| | (3,4-difluoro-2- | | | | |
| | methoxyphenyl)-4,5- | | | | |
| | dimethyl-N- | | | | |
| | (tetrazolo[1,5- | | | | |
| | a]pyridin-7-yl)-5- | | | | |
| | (trifluoromethyl) | | | | |
| | tetrahydrofuran-2- | | | | |
| | carboxamide | | | | |
| | (first eluting isomer by | | | | |
| | SFC using Whelk-O1 | | | | |
| | column) | | | | |
| 32 | rel-(2R,3S,4S,5R)-3- | 471.381 | 472.1 | 3.29 | ¹H NMR (500 MHZ, |
| | (3,4-difluoro-2- | | | | Chloroform-d) δ 8.77-8.71 |
| | methoxyphenyl)-4,5- | | | | (m, 2H), 8.35 (dd, J = 2.1, |
| | dimethyl-N- | | | | 0.8 Hz, 1H), 7.45 (dd, J = |
| | (tetrazolo[1,5- | | | | 7.5, 2.1 Hz, 1H), 7.14- |
| | a]pyridin-7-yl)-5- | | | | 7.06 (m, 1H), 6.95 (td, J = |
| | (trifluoromethyl) | | | | 9.1, 7.4 Hz, 1H), 5.09 (d, |
| | tetrahydrofuran-2- | | | | J = 11.0 Hz, 1H), 4.16 (dd, |
| | carboxamide | | | | J = 11.1, 8.1 Hz, 1H), 4.04 |
| | (second eluting isomer | | | | (d, J = 2.8 Hz, 3H), 2.80 (p, |
| | by SFC using Whelk- | | | | J = 7.7 Hz, 1H), 1.73 (d, J = |
| | O1 column) | | | | 1.1 Hz, 3H), 0.83 (dq, J = |
| | | | | | 7.4, 2.4 Hz, 3H) ppm. |
| 33 | rel-(2S,3R,4R,5S)-3- | 505.435 | 506 | 3.35 | ¹H NMR (500 MHZ, |
| | (3,4-difluoro-2- | | | | Chloroform-d) δ 8.80 (d, |
| | methoxyphenyl)-N- | | | | J = 2.1 Hz, 1H), 8.45 (s, 1H), |
| | (6-(2- | | | | 7.57 (d, J = 2.1 Hz, 1H), |
| | methoxyethoxy) | | | | 7.05 (ddd, J = 8.2, 5.4, 2.1 |
| | pyridazin-4-yl)-4,5- | | | | Hz, 1H), 6.90 (td, J = 9.3, |
| | dimethyl-5- | | | | 7.4 Hz, 1H), 5.01 (d, J = |
| | (trifluoromethyl) | | | | 10.9 Hz, 1H), 4.70-4.61 |
| | tetrahydrofuran-2- | | | | (m, 2H), 4.08 (dd, J = 11.0, |
| | carboxamide | | | | 8.2 Hz, 1H), 4.00 (d, J = 2.8 |
| | (first eluting isomer by | | | | Hz, 3H), 3.78-3.72 (m, |
| | SFC using AS-H | | | | 2H), 3.41 (s, 3H), 2.80- |
| | column) | | | | 2.70 (m, 1H), 1.70-1.65 |
| | | | | | (m, 3H), 0.83-0.77 (m, |
| | | | | | 3H) ppm. |
| 34 | rel-(2R,3S,4S,5R)-3- | 505.435 | 506 | 3.35 | ¹H NMR (500 MHz, |
| | (3,4-difluoro-2- | | | | Chloroform-d) δ 8.79 (d, |
| | methoxyphenyl)-N- | | | | J = 2.2 Hz, 1H), 8.43 (s, 1H), |
| | (6-(2-methoxyethoxy) | | | | 7.56 (d, J = 2.2 Hz, 1H), |
| | pyridazin-4-yl)-4,5- | | | | 7.05 (ddd, J = 8.6, 5.5, 2.0 |
| | dimethyl-5- | | | | Hz, 1H), 6.90 (td, J = 9.3, |
| | (trifluoromethyl) | | | | 7.4 Hz, 1H), 5.01 (d, J = |
| | tetrahydrofuran-2- | | | | 10.9 Hz, 1H), 4.71-4.59 |
| | carboxamide | | | | (m, 2H), 4.08 (dd, J = 11.0, |
| | (second eluting isomer | | | | 8.2 Hz, 1H), 4.00 (d, J = 2.7 |
| | by SFC using AS-H | | | | Hz, 3H), 3.81-3.72 (m, |
| | column) | | | | 2H), 3.41 (s, 3H), 2.81- |
| | | | | | 2.69 (m, 1H), 1.68 (d, J = |
| | | | | | 1.1 Hz, 3H), 0.79 (dq, J = |
| | | | | | 7.3, 2.3 Hz, 3H) ppm. |
| 35 | rel-(2S,3R,4R,5S)-3- | 431.357 | 432.5 | 3.34 | ¹H NMR (400 MHZ, |
| | (3,4-difluoro-2- | | | | Chloroform-d) δ 9.01-8.91 |
| | methoxyphenyl)-4,5- | | | | (m, 2H), 8.63 (d, J = 5.8 |
| | dimethyl-N- | | | | Hz, 1H), 8.12 (dd, J = 5.7, |
| | (pyrimidin-4-yl)-5- | | | | 1.4 Hz, 1H), 7.10 (ddd, J = |
| | (trifluoromethyl) | | | | 8.2, 5.5, 2.2 Hz, 1H), 6.99- |
| | tetrahydrofuran-2- | | | | 6.84 (m, 1H), 5.04 (d, J = |
| | carboxamide | | | | 11.1 Hz, 1H), 4.20-4.07 |
| | (first eluting isomer by | | | | (m, 1H), 4.02 (d, J = 2.8 |
| | SFC using Whelk-O1 | | | | Hz, 3H), 2.84-2.70 (m, |
| | column) | | | | 1H), 1.79-1.60 (m, 3H), |
| | | | | | 0.80 (ddq, J = 12.5, 7.4, 2.3 |
| | | | | | Hz, 3H) ppm. |
| 36 | rel-(2R,3S,4S,5R)-3- | 431.357 | 432.6 | 3.34 | ¹H NMR (400 MHZ, |
| | (3,4-difluoro-2- | | | | Chloroform-d) δ 9.00-8.91 |
| | methoxyphenyl)-4,5- | | | | (m, 2H), 8.63 (d, J = 5.8 |
| | dimethyl-N- | | | | Hz, 1H), 8.12 (dd, J = 5.7, |
| | (pyrimidin-4-yl)-5- | | | | 1.3 Hz, 1H), 7.10 (ddd, J = |
| | (trifluoromethyl) | | | | 8.2, 5.5, 2.2 Hz, 1H), 6.93 |
| | tetrahydrofuran-2- | | | | (td, J = 9.2, 7.4 Hz, 1H), |
| | carboxamide | | | | 5.04 (d, J = 11.0 Hz, 1H), |
| | (second eluting isomer | | | | 4.12 (dd, J = 11.0, 8.1 Hz, |
| | by SFC using Whelk- | | | | 1H), 4.02 (d, J = 2.8 Hz, |
| | O1 column) | | | | 3H), 2.77 (p, J = 7.7 Hz, |
| | | | | | 1H), 1.72 (d, J = 1.2 Hz, |
| | | | | | 3H), 0.81 (dq, J = 7.5, 2.4 |
| | | | | | Hz, 3H) ppm. |
| 37 | rel-(2S,3R,4R,5S)-3- | 431.357 | 432.2 | 3.17 | ¹H NMR (400 MHZ, |
| | (3,4-difluoro-2- | | | | Chloroform-d) δ 9.02 (d, |
| | methoxyphenyl)-4,5- | | | | J = 4.3 Hz, 3H), 8.41 (s, 1H), |
| | dimethyl-N- | | | | 7.09 (ddd, J = 8.2, 5.5, 2.2 |
| | (pyrimidin-5-yl)-5- | | | | Hz, 1H), 6.93 (td, J = 9.2, |
| | (trifluoromethyl) | | | | 7.4 Hz, 1H), 5.07 (d, J = |
| | tetrahydrofuran-2- | | | | 11.0 Hz, 1H), 4.13 (dd, J = |
| | carboxamide | | | | 11.0, 8.1 Hz, 1H), 4.03 (d, |
| | (first eluting isomer by | | | | J = 2.8 Hz, 3H), 2.79 (p, J = |
| | SFC using Whelk-O1 | | | | 7.6 Hz, 1H), 1.71 (d, J = 1.1 |
| | column) | | | | Hz, 3H), 0.82 (dq, J = 7.4, |
| | | | | | 2.4 Hz, 3H) ppm. |
| 38 | rel-(2R,3S,4S,5R)-3- | 431.357 | 432.2 | 3.17 | ¹H NMR (400 MHZ, |
| | (3,4-difluoro-2- | | | | Chloroform-d) δ 9.02 (d, |
| | methoxyphenyl)-4,5- | | | | J = 2.9 Hz, 3H), 8.39 (s, 1H), |
| | dimethyl-N- | | | | 7.10 (ddd, J = 8.2, 5.6, 2.2 |
| | (pyrimidin-5-yl)-5- | | | | Hz, 1H), 6.93 (td, J = 9.2, |
| | (trifluoromethyl) | | | | 7.4 Hz, 1H), 5.07 (d, J = |
| | tetrahydrofuran-2- | | | | 11.0 Hz, 1H), 4.13 (dd, J = |
| | carboxamide | | | | 11.0, 8.1 Hz, 1H), 4.04 (d, |
| | (second eluting isomer | | | | J = 2.8 Hz, 3H), 2.79 (p, J = |
| | by SFC using Whelk- | | | | 7.7 Hz, 1H), 1.71 (s, 3H), |
| | O1 column) | | | | 0.82 (dq, J = 7.4, 2.4 Hz, |
| | | | | | 3H) ppm. |
| 39 | rel-(2S,3R,4R,5S)-3- | 505.415 | 506.1 | 3.11 | ¹H NMR (500 MHZ, |
| | (3,4-difluoro-2- | | | | Chloroform-d) δ 8.48 (s, |
| | methoxyphenyl)-N-(4- | | | | 1H), 7.72-7.63 (m, 4H), |
| | (dimethylphosphoryl) | | | | 7.10 (ddd, J = 8.1, 5.5, 2.1 |
| | phenyl)-4,5-dimethyl-5- | | | | Hz, 1H), 6.90 (td, J = 9.2, |
| | (trifluoromethyl) | | | | 7.4 Hz, 1H), 5.02 (d, J = |
| | tetrahydrofuran-2- | | | | 10.9 Hz, 1H), 4.10 (dd, J = |
| | carboxamide | | | | 10.9, 8.0 Hz, 1H), 4.00 (d, |
| | (first eluting isomer by | | | | J = 2.7 Hz, 3H), 2.75 (p, J = |
| | SFC using Whelk-O1 | | | | 7.7 Hz, 1H), 1.72 (d, J = 1.5 |
| | column) | | | | Hz, 3H), 1.69 (dd, J = 8.2, |
| | | | | | 1.3 Hz, 6H), 0.79 (dq, J = |
| | | | | | 7.4, 2.3 Hz, 3H) ppm. |
| 40 | rel-(2R,3S,4S,5R)-3- | 505.415 | 506.1 | 3.11 | ¹H NMR (500 MHZ, |
| | (3,4-difluoro-2- | | | | Chloroform-d) δ 8.49 (s, |
| | methoxyphenyl)-N-(4- | | | | 1H), 7.73-7.63 (m, 4H), |
| | (dimethylphosphoryl) | | | | 7.09 (ddd, J = 8.2, 5.5, 2.1 |
| | phenyl)-4,5-dimethyl-5- | | | | Hz, 1H), 6.90 (td, J = 9.2, |
| | (trifluoromethyl) | | | | 7.4 Hz, 1H), 5.02 (d, J = |
| | tetrahydrofuran-2- | | | | 10.9 Hz, 1H), 4.10 (dd, J = |
| | carboxamide | | | | 10.9, 8.0 Hz, 1H), 3.99 (d, |
| | (second eluting isomer | | | | J = 2.7 Hz, 3H), 2.74 (p, J = |
| | by SFC using Whelk- | | | | 7.6 Hz, 1H), 1.72 (d, J = 1.5 |
| | O1 column) | | | | Hz, 3H), 1.69 (dd, J = 8.2, |
| | | | | | 1.3 Hz, 6H), 0.79 (dq, J = |
| | | | | | 7.4, 2.3 Hz, 3H) ppm. |
| 41 | rel-(2S,3R,4R,5S)-3- | 505.415 | 506.1 | 3.1 | ¹H NMR (500 MHZ, |
| | (3,4-difluoro-2- | | | | Chloroform-d) δ 8.52 (s, |
| | methoxyphenyl)-N-(3- | | | | 1H), 7.91 (ddt, J = 8.0, 2.5, |
| | (dimethylphosphoryl) | | | | 1.3 Hz, 1H), 7.84 (dt, J = |
| | phenyl)-4,5-dimethyl-5- | | | | 12.8, 1.8 Hz, 1H), 7.49- |
| | (trifluoromethyl) | | | | 7.35 (m, 2H), 7.10 (ddd, J = |
| | tetrahydrofuran-2- | | | | 8.4, 5.5, 2.1 Hz, 1H), 6.90 |
| | carboxamide | | | | (td, J = 9.3, 7.5 Hz, 1H), |
| | (first eluting isomer by | | | | 5.02 (d, J = 10.9 Hz, 1H), |
| | SFC using Whelk-O1 | | | | 4.09 (dd, J = 10.9, 8.0 Hz, |
| | column) | | | | 1H), 4.00 (d, J = 2.7 Hz, |
| | | | | | 3H), 2.75 (p, J = 7.6 Hz, |
| | | | | | 1H), 1.73 (d, J = 13.1 Hz, |
| | | | | | 6H), 1.68 (d, J = 1.1 Hz, |
| | | | | | 3H), 0.78 (dt, J = 7.4, 2.4 |
| | | | | | Hz, 3H) ppm. |
| 42 | rel-(2R,3S,4S,5R)-3- | 505.415 | 506.1 | 3.1 | ¹H NMR (500 MHZ, |
| | (3,4-difluoro-2- | | | | Chloroform-d) δ 8.56 (s, |
| | methoxyphenyl)-N-(3- | | | | 1H), 7.92 (ddt, J = 8.1, 2.4, |
| | (dimethylphosphoryl) | | | | 1.2 Hz, 1H), 7.85 (dt, J = |
| | phenyl)-4,5-dimethyl-5- | | | | 12.6, 1.7 Hz, 1H), 7.48- |
| | (trifluoromethyl) | | | | 7.36 (m, 2H), 7.10 (ddd, J = |
| | tetrahydrofuran-2- | | | | 8.3, 5.5, 2.1 Hz, 1H), 6.90 |
| | carboxamide | | | | (td, J = 9.3, 7.5 Hz, 1H), |
| | (second eluting isomer | | | | 5.02 (d, J = 10.9 Hz, 1H), |
| | by SFC using Whelk- | | | | 4.10 (dd, J = 10.9, 8.0 Hz, |
| | O1 column) | | | | 1H), 4.00 (d, J = 2.7 Hz, |
| | | | | | 3H), 2.75 (p, J = 7.7 Hz, |
| | | | | | 1H), 1.73 (d, J = 13.0 Hz, |
| | | | | | 6H), 1.68 (d, J = 1.1 Hz, |
| | | | | | 3H), 0.79 (dq, J = 7.4, 2.3 |
| | | | | | Hz, 3H) ppm. |

The following compounds were made using the method described in Example 1, except that different coupling partners were used in the amide coupling step 11 and General Method B was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 43 | rel-(2S,3R,4R,5S)-3- | 490.42 | 491.7 | 3.00 | ¹H NMR (500 MHZ, |
| | (3,4-difluoro-2- | | | | DMSO-d6) δ 10.57 (s, 1H), |
| | methoxyphenyl)-N-(2- | | | | 8.35 (d, J = 5.5 Hz, 1H), |
| | (−1,2- | | | | 7.72 (d, J = 2.1 Hz, 1H), |
| | dihydroxyethyl)pyridin- | | | | 7.52 (dd, J = 5.5, 2.1 Hz, |
| | 4-yl)-4,5-dimethyl-5- | | | | 1H), 7.21-7.11 (m, 2H), |
| | (trifluoromethyl)tetrahy | | | | 5.38 (d, J = 4.7 Hz, 1H), |
| | drofuran-2-carboxamide | | | | 5.09 (d, J = 10.3 Hz, 1H), |
| | (precursor was first | | | | 4.66 (t, J = 5.9 Hz, 1H), |
| | eluting isomer by SFC | | | | 4.53 (dt, J = 6.7, 4.3 Hz, |
| | using Lux-Cellulose-2 | | | | 1H), 4.25 (dd, J = 10.3, 7.7 |
| | column; 2-(2,2- | | | | Hz, 1H), 3.96 (d, J = 2.1 |
| | dimethyl-1,3-dioxolan- | | | | Hz, 3H), 3.68-3.64 (m, |
| | 4-yl)pyridin-4-amine | | | | 1H), 3.47-3.42 (m, 1H), |
| | (first eluting isomer by | | | | 2.78 (p, J = 7.6 Hz, 1H), |
| | SFC using a Chiralpak | | | | 1.61 (s, 3H), 0.73 (d, J = |
| | ID column used in step | | | | 7.4 Hz, 3H) ppm. |
| | 11) | | | | |
| 44 | rel-(2R,3S,4S,5R)-3- | 490.42 | 491.6 | 3.01 | ¹H NMR (400 MHZ, |
| | (3,4-difluoro-2- | | | | DMSO-d6) δ 10.56 (s, 1H), |
| | methoxyphenyl)-N-(2- | | | | 8.35 (d, J = 5.5 Hz, 1H), |
| | (−1,2- | | | | 7.72 (d, J = 2.1 Hz, 1H), |
| | dihydroxyethyl)pyridin- | | | | 7.52 (dd, J = 5.6, 2.1 Hz, |
| | 4-yl)-4,5-dimethyl-5- | | | | 1H), 7.21-7.11 (m, 2H), |
| | (trifluoromethyl)tetrahy | | | | 5.39 (d, J = 4.7 Hz, 1H), |
| | drofuran-2-carboxamide | | | | 5.09 (d, J = 10.3 Hz, 1H), |
| | (precursor was second | | | | 4.66 (t, J = 5.9 Hz, 1H), |
| | eluting isomer by SFC | | | | 4.53 (dt, J = 6.8, 4.3 Hz, |
| | using Lux-Cellulose-2 | | | | 1H), 4.25 (dd, J = 10.3, 7.6 |
| | column; 2-(2,2- | | | | Hz, 1H), 3.96 (d, J = 2.2 |
| | dimethyl-1,3-dioxolan- | | | | Hz, 3H), 3.66 (ddd, J = |
| | 4-yl)pyridin-4-amine | | | | 10.4, 6.0, 4.1 Hz, 1H), 3.44 |
| | (first eluting isomer by | | | | (dt, J = 11.0, 6.4 Hz, 1H), |
| | SFC using a Chiralpak | | | | 2.78 (p, J = 7.5 Hz, 1H), |
| | ID column) used in step | | | | 1.60 (s, 3H), 0.73 (d, J = |
| | 11)) | | | | 7.3 Hz, 3H) ppm. |
| 45 | rel-(2S,3R,4R,5S)-3- | 490.42 | 491.65 | 2.30 | ¹H NMR (500 MHZ, |
| | (3,4-difluoro-2- | | | | DMSO-d6) δ 10.56 (s, 1H), |
| | methoxyphenyl)-N-(2- | | | | 8.35 (d, J = 5.4 Hz, 1H), |
| | (−1,2- | | | | 7.72 (d, J = 2.1 Hz, 1H), |
| | dihydroxyethyl)pyridin- | | | | 7.52 (dd, J = 5.6, 2.1 Hz, |
| | 4-yl)-4,5-dimethyl-5- | | | | 1H), 7.21-7.11 (m, 2H), |
| | (trifluoromethyl) | | | | 5.38 (d, J = 4.7 Hz, 1H), |
| | tetrahydrofuran-2- | | | | 5.09 (d, J = 10.3 Hz, 1H), |
| | carboxamide | | | | 4.66 (t, J = 5.9 Hz, 1H), |
| | (precursor was first | | | | 4.53 (dt, J = 6.7, 4.3 Hz, |
| | eluting isomer by SFC | | | | 1H), 4.25 (dd, J = 10.3, 7.7 |
| | using AS-H column; 2- | | | | Hz, 1H), 3.96 (d, J = 2.1 |
| | (2,2-dimethyl-1,3- | | | | Hz, 3H), 3.66 (ddd, J = |
| | dioxolan-4-yl)pyridin-4- | | | | 10.9, 5.9, 4.0 Hz, 1H), 3.44 |
| | amine (second eluting | | | | (ddd, J = 11.0, 6.9, 5.9 Hz, |
| | isomer by SFC using a | | | | 1H), 2.78 (p, J = 7.5 Hz, |
| | Chiralpak ID column) | | | | 1H), 1.60 (s, 3H), 0.73 (d, |
| | used in step 11) | | | | J = 5.4 Hz, 3H) ppm. |
| 46 | rel-(2R,3S,4S,5R)-3- | 490.42 | 491.633 | 3.01 | ¹H NMR (500 MHZ, |
| | (3,4-difluoro-2- | | | | DMSO-d6) δ 10.56 (s, 1H), |
| | methoxyphenyl)-N-(2- | | | | 8.35 (d, J = 5.5 Hz, 1H), |
| | (−1,2- | | | | 7.72 (d, J = 2.1 Hz, 1H), |
| | dihydroxyethyl)pyridin- | | | | 7.52 (dd, J = 5.5, 2.2 Hz, |
| | 4-yl)-4,5-dimethyl-5- | | | | 1H), 7.21-7.11 (m, 2H), |
| | (trifluoromethyl)tetrahy | | | | 5.38 (d, J = 4.7 Hz, 1H), |
| | drofuran-2-carboxamide | | | | 5.09 (d, J = 10.3 Hz, 1H), |
| | (precursor was second | | | | 4.66 (t, J = 5.9 Hz, 1H), |
| | eluting isomer by SFC | | | | 4.53 (dt, J = 6.8, 4.3 Hz, |
| | using AS-H column; 2- | | | | 1H), 4.25 (dd, J = 10.3, 7.7 |
| | (2,2-dimethyl-1,3- | | | | Hz, 1H), 3.96 (d, J = 2.1 |
| | dioxolan-4-yl)pyridin-4- | | | | Hz, 3H), 3.66 (ddd, J = |
| | amine (second eluting | | | | 11.0, 6.0, 4.0 Hz, 1H), 3.47- |
| | isomer by SFC using a | | | | 3.42 (m, 1H), 2.78 (p, J = |
| | Chiralpak ID column | | | | 7.6 Hz, 1H), 1.61 (s, 3H), |
| | used in step 11) | | | | 0.73 (d, J = 6.9 Hz, 3H) |
| | | | | | ppm. |

The following compounds were made using the method described in Example 1, except that a different coupling partner was used in the amide coupling step 11 and conditions similar to General Method N were used for silyl deprotection as the final step: In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 47 | rel-(2S,3R,4R,5S)-3- | 488.448 | | 3.35 | ¹H NMR (500 MHZ, DMSO- |
| | (3,4-difluoro-2- | | | | d6) δ 10.55 (s, 1H), 8.35 (d, |
| | methoxyphenyl)-N-(2- | | | | J = 5.5 Hz, 1H), 7.87 (d, J = |
| | (2-hydroxypropan-2- | | | | 2.1 Hz, 1H), 7.50 (dd, J = |
| | yl)pyridin-4-yl)-4,5- | | | | 5.6, 2.1 Hz, 1H), 7.22-7.11 |
| | dimethyl-5- | | | | (m, 2H), 5.17 (s, 1H), 5.09 |
| | (trifluoromethyl) | | | | (d, J = 10.2 Hz, 1H), 4.26 |
| | tetrahydrofuran-2- | | | | (dd, J = 10.4, 7.6 Hz, 1H), |
| | carboxamide | | | | 3.96 (d, J = 2.2 Hz, 3H), 2.78 |
| | (precursor was first | | | | (p, J = 7.5 Hz, 1H), 1.61 (s, |
| | eluting isomer by SFC | | | | 3H), 1.40 (s, 6H), 0.83-0.65 |
| | using Whelk-O1 | | | | (m, 3H) ppm. |
| | column) | | | | |
| 48 | rel-(2R,3S,4S,5R)-3- | 488.448 | 489.7 | 3.35 | ¹H NMR (500 MHZ, DMSO- |
| | (3,4-difluoro-2- | | | | d6) δ 8.52-8.36 (m, 1H), |
| | methoxyphenyl)-N-(2- | | | | 8.12-7.98 (m, 1H), 7.87- |
| | (2-hydroxypropan-2- | | | | 7.71 (m, 1H), 7.19-7.12 (m, |
| | yl)pyridin-4-yl)-4,5- | | | | 2H), 5.16 (d, J = 10.2 Hz, |
| | dimethyl-5- | | | | 1H), 4.26 (dd, J = 10.2, 7.7 |
| | (trifluoromethyl) | | | | Hz, 1H), 3.94 (d, J = 2.1 Hz, |
| | tetrahydrofuran-2- | | | | 3H), 2.77 (p, J = 7.7 Hz, 1H), |
| | carboxamide | | | | 1.59 (s, 3H), 1.46 (s, 6H), |
| | (precursor was second | | | | 0.77-0.65 (m, 3H) ppm. |
| | eluting isomer by SFC | | | | |
| | using Whelk-O1 | | | | |
| | column) | | | | |

The following compounds were made using the method described in Example 1, except that different coupling partners were used in the amide coupling step 11 and General Method D was used as the penultimate step before SFC. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 49 | rel-(2S,3R,4R,5S)-3- | 508.459 | 509.1 | 3.38 | ¹H NMR (500 MHZ, |
| | (3,4-difluoro-2- | | | | Chloroform-d) δ 8.70 (s, |
| | methoxyphenyl)-4,5- | | | | 1H), 8.59 (d, J = 5.5 Hz, |
| | dimethyl-N-(2- | | | | 1H), 8.03 (dd, J = 5.5, 2.1 |
| | (methylsulfonyl)pyridin- | | | | Hz, 1H), 8.00 (d, J = 2.0 |
| | 4-yl)-5- | | | | Hz, 1H), 7.06 (ddd, J = 8.1, |
| | (trifluoromethyl) | | | | 5.5, 2.1 Hz, 1H), 6.91 (td, |
| | tetrahydrofuran-2- | | | | J = 9.2, 7.4 Hz, 1H), 5.03 (d, |
| | carboxamide | | | | J = 11.1 Hz, 1H), 4.08 (dd, |
| | (first eluting isomer by | | | | J = 11.1, 8.1 Hz, 1H), 4.01 |
| | SFC using As-H | | | | (d, J = 2.8 Hz, 3H), 3.22 (s, |
| | column) | | | | 3H), 2.76 (p, J = 7.7 Hz, |
| | | | | | 1H), 1.69 (d, J = 1.2 Hz, |
| | | | | | 3H), 0.79 (dq, J = 7.4, 2.3 |
| | | | | | Hz, 3H) ppm. |
| 50 | rel-(2R,3S,4S,5R)-3- | 508.459 | 509.1 | 3.38 | ¹H NMR (500 MHz, |
| | (3,4-difluoro-2- | | | | Chloroform-d) δ 8.78-8.68 |
| | methoxyphenyl)-4,5- | | | | (m, 1H), 8.59 (d, J = 5.4 |
| | dimethyl-N-(2- | | | | Hz, 1H), 8.03 (dd, J = 5.5, |
| | (methylsulfonyl)pyridin- | | | | 2.1 Hz, 1H), 8.00 (d, J = 2.0 |
| | 4-yl)-5- | | | | Hz, 1H), 7.06 (ddd, J = 8.0, |
| | (trifluoromethyl) | | | | 5.4, 2.1 Hz, 1H), 6.91 (td, |
| | tetrahydrofuran-2- | | | | J = 9.2, 7.4 Hz, 1H), 5.03 (d, |
| | carboxamide | | | | J = 11.1 Hz, 1H), 4.08 (dd, |
| | (second eluting isomer | | | | J = 11.1, 8.1 Hz, 1H), 4.01 |
| | by SFC using As-H | | | | (d, J = 2.8 Hz, 3H), 3.22 (s, |
| | column) | | | | 3H), 2.76 (p, J = 7.7 Hz, |
| | | | | | 1H), 1.69 (d, J = 1.3 Hz, |
| | | | | | 3H), 0.79 (dq, J = 7.4, 2.3 |
| | | | | | Hz, 3H) ppm. |
| 51 | rel-(2R,3S,4S,5R)-3- | 522.485 | 523 | 3.44 | ¹H NMR (500 MHZ, |
| | (3,4-difluoro-2- | | | | Chloroform-d) 8.67 (s, 1H), |
| | methoxyphenyl)-N-(2- | | | | 8.61 (d, J = 5.4 Hz, 1H), 8.05 |
| | (ethylsulfonyl)pyridin- | | | | (dd, J = 5.5, 2.1 Hz, 1H), |
| | 4-yl)-4,5-dimethyl-5- | | | | 7.97 (dd, J = 2.2, 0.5 Hz, |
| | (trifluoromethyl) | | | | 1H), 7.06 (ddd, J = 7.8, 5.3, |
| | tetrahydrofuran-2- | | | | 2.0 Hz, 1H), 6.95-6.87 (m, |
| | carboxamide | | | | 1H), 5.03 (d, J = 11.1 Hz, |
| | (first eluting isomer by | | | | 1H), 4.08 (dd, J = 11.1, 8.1 |
| | SFC using Lux | | | | Hz, 1H), 4.01 (d, J = 2.9 Hz, |
| | Cellulose-2 column) | | | | 3H), 3.42 (qd, J = 7.4, 1.0 |
| | | | | | Hz, 2H), 2.76 (t, J = 7.8 Hz, |
| | | | | | 1H), 1.70 (d, J = 1.1 Hz, |
| | | | | | 3H), 1.30 (t, J = 7.4 Hz, 3H), |
| | | | | | 0.79 (dt, J = 7.5, 2.4 Hz, 3H) |
| | | | | | ppm. |
| 52 | rel-(2S,3R,4R,5S)-3- | 522.485 | 523 | 3.44 | ¹H NMR (500 MHZ, |
| | (3,4-difluoro-2- | | | | Chloroform-d) 8.69 (s, 1H), |
| | methoxyphenyl)-N-(2- | | | | 8.61 (d, J = 5.5 Hz, 1H), 8.05 |
| | (ethylsulfonyl)pyridin- | | | | (dd, J = 5.5, 2.2 Hz, 1H), |
| | 4-yl)-4,5-dimethyl-5- | | | | 7.97 (dd, J = 2.2, 0.6 Hz, |
| | (trifluoromethyl) | | | | 1H), 7.06 (td, J = 5.8, 2.7 |
| | tetrahydrofuran-2- | | | | Hz, 1H), 6.97-6.85 (m, 1H), |
| | carboxamide | | | | 5.03 (d, J = 11.1 Hz, 1H), |
| | (second eluting isomer | | | | 4.08 (dd, J = 11.1, 8.1 Hz, |
| | by SFC using Lux | | | | 1H), 4.01 (d, J = 2.8 Hz, |
| | Cellulose-2 column) | | | | 3H), 3.41 (td, J = 7.5, 1.0 |
| | | | | | Hz, 2H), 2.83-2.70 (m, 1H), |
| | | | | | 1.70 (d, J = 1.1 Hz, 3H), 1.30 |
| | | | | | (t, J = 7.4 Hz, 3H), 0.86-0.73 |
| | | | | | (m, 3H) ppm. |

The following compounds were made using the method described in Example 1, except that 2-(methylthio)pyridin-4-amine was used in the amide coupling step 11 and General Method F was used in place of step 12. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 6 | rel-(2S,3R,4R,5S)-3- | 492.459 | 493.2 | 3.21 | ¹H NMR (500 MHZ, |
| | (3,4-difluoro-2- | | | | Chloroform-d) δ 8.91 (s, 1H), |
| | methoxyphenyl)-4,5- | | | | 8.49 (d, J = 5.5 Hz, 1H), 8.09 |
| | dimethyl-N-(2- | | | | (dd, J = 5.5, 2.1 Hz, 1H), 7.82 |
| | (methylsulfinyl) | | | | (d, J = 2.0 Hz, 1H), 7.07 (ddd, |
| | pyridin-4-yl)-5- | | | | J = 8.0, 5.5, 2.0 Hz, 1H), 6.90 |
| | (trifluoromethyl) | | | | (td, J = 9.2, 7.4 Hz, 1H), 5.03 |
| | tetrahydrofuran-2- | | | | (dd, J = 11.0, 3.0 Hz, 1H), 4.10 |
| | carboxamide | | | | (dd, J = 11.0, 8.1 Hz, 1H), 4.01 |
| | (first eluting isomer by | | | | (d, J = 2.8 Hz, 3H), 2.85 (s, |
| | SFC using AS-H | | | | 3H), 2.76 (p, J = 7.7 Hz, 1H), |
| | column then first | | | | 1.69 (d, J = 1.5 Hz, 3H), 0.80 |
| | eluting isomer by SFC | | | | (dq, J = 7.3, 2.3 Hz, 3H) ppm. |
| | on Whelk-O1 column) | | | | |
| 7 | rel-(2S,3R,4R,5S)-3- | 492.459 | 493.2 | 3.2 | ¹H NMR (500 MHZ, |
| | (3,4-difluoro-2- | | | | Chloroform-d) δ 8.49 (d, J = |
| | methoxyphenyl)-4,5- | | | | 5.5 Hz, 1H), 8.06 (dd, J = 5.5, |
| | dimethyl-N-(2- | | | | 2.2 Hz, 1H), 7.80 (d, J = 2.1 |
| | (methylsulfinyl) | | | | Hz, 1H), 7.06 (ddd, J = 8.0, |
| | pyridin-4-yl)-5- | | | | 5.4, 2.0 Hz, 1H), 6.90 (td, J = |
| | (trifluoromethyl) | | | | 9.2, 7.4 Hz, 1H), 5.03 (d, J = |
| | tetrahydrofuran-2- | | | | 11.0 Hz, 1H), 4.10 (dd, J = |
| | carboxamide | | | | 11.0, 8.1 Hz, 1H), 4.01 (d, J = |
| | (first eluting isomer by | | | | 2.8 Hz, 3H), 2.85 (s, 3H), 2.75 |
| | SFC using AS-H | | | | (p, J = 7.7 Hz, 1H), 1.69 (d, J = |
| | column then second | | | | 1.3 Hz, 3H), 0.87-0.76 (m, |
| | eluting isomer by SFC | | | | 3H) ppm. |
| | on Whelk-O1 column) | | | | |
| 8 | rel-(2R,3S,4S,5R)-3- | 492.459 | 493.1 | 3.21 | ¹H NMR (500 MHZ, |
| | (3,4-difluoro-2- | | | | Chloroform-d) δ 8.88 (d, J = |
| | methoxyphenyl)-4,5- | | | | 8.2 Hz, 1H), 8.49 (d, J = 5.5 |
| | dimethyl-N-(2- | | | | Hz, 1H), 8.07 (ddd, J = 9.3, |
| | (methylsulfinyl) | | | | 5.5, 2.2 Hz, 1H), 7.80 (d, J = |
| | pyridin-4-yl)-5- | | | | 2.1 Hz, 1H), 7.12-7.03 (m, |
| | (trifluoromethyl) | | | | 1H), 6.90 (td, J = 9.2, 7.4 Hz, |
| | tetrahydrofuran-2- | | | | 1H), 5.03 (dd, J = 11.0, 3.0 Hz, |
| | carboxamide | | | | 1H), 4.10 (ddd, J = 10.4, 8.2, |
| | (second eluting isomer | | | | 1.7 Hz, 1H), 4.01 (d, J = 2.7 |
| | by SFC using AS-H | | | | Hz, 3H), 2.85 (d, J = 2.5 Hz, |
| | column then first | | | | 3H), 2.79-2.69 (m, 1H), 1.72- |
| | eluting isomer by SFC | | | | 1.66 (m, 3H), 0.80 (dq, J = |
| | on Whelk-O1 column) | | | | 7.4, 2.4 Hz, 3H) ppm. |
| 9 | rel-(2R,3S,4S,5R)-3- | 492.459 | 493.1 | 3.2 | ¹H NMR (500 MHZ, |
| | (3,4-difluoro-2- | | | | Chloroform-d) δ 8.85 (s, 1H), |
| | methoxyphenyl)-4,5- | | | | 8.49 (d, J = 5.5 Hz, 1H), 8.06 |
| | dimethyl-N-(2- | | | | (dd, J = 5.5, 2.2 Hz, 1H), 7.79 |
| | (methylsulfinyl) | | | | (d, J = 2.1 Hz, 1H), 7.07 (ddd, |
| | pyridin-4-yl)-5- | | | | J = 8.2, 5.4, 2.0 Hz, 1H), 6.90 |
| | (trifluoromethyl) | | | | (td, J = 9.2, 7.4 Hz, 1H), 5.03 |
| | tetrahydrofuran-2- | | | | (d, J = 11.0 Hz, 1H), 4.10 (dd, |
| | carboxamide | | | | J = 11.1, 8.1 Hz, 1H), 4.01 (d, |
| | (second eluting isomer | | | | J = 2.8 Hz, 3H), 2.85 (d, J = |
| | by SFC using AS-H | | | | 3.1 Hz, 3H), 2.76 (p, J = 7.6 |
| | column then second | | | | Hz, 1H), 1.69 (d, J = 1.4 Hz, |
| | eluting isomer by SFC | | | | 3H), 0.79 (dt, J = 7.5, 2.3 Hz, |
| | on Whelk-O1 column) | | | | 3H) ppm. |

The following compounds were made using the method described in Example 1, except that 2-(methylthio)pyridin-4-amine was used in the amide coupling step 11. This was followed by General Method G (using 1:1 MeOH and DCM as solvent for step 1) in place of step 12. Enantiomers were separated by chiral SFC as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 53 | rel-(2S,3R,4R,5S)-3- | 507.474 | 508.6 | 3.07 | 'H NMR (500 MHZ, |
| | (3,4-difluoro-2- | | | | Methanol-d₄) δ 8.59 (dd, J = |
| | methoxyphenyl)-4,5- | | | | 5.5, 0.6 Hz, 1H), 8.46 (dd, |
| | dimethyl-N-(2-(S- | | | | J = 2.0, 0.6 Hz, 1H), 7.90 (dd, |
| | methylsulfonimidoyl) | | | | J = 5.5, 2.1 Hz, 1H), 7.14 |
| | pyridin-4-yl)-5- | | | | (ddd, J = 8.2, 5.5, 2.1 Hz, |
| | (trifluoromethyl) | | | | 1H), 7.04-6.95 (m, 1H), |
| | tetrahydrofuran-2- | | | | 5.11 (d, J = 10.3 Hz, 1H), |
| | carboxamide | | | | 4.35 (dd, J = 10.3, 8.0 Hz, |
| | (first eluting isomer by | | | | 1H), 4.02 (d, J = 2.3 Hz, 3H), |
| | SFC using AD-H | | | | 3.24 (s, 3H), 2.82 (p, J = 7.6 |
| | column then achiral | | | | Hz, 1H), 1.68 (d, J = 1.2 Hz, |
| | SFC purification using | | | | 3H), 0.84 (dq, J = 7.4, 2.4 |
| | Whelk-O1 column) | | | | Hz, 3H) ppm. |
| 54 | rel-(2R,3S,4S,5R)-3- | 507.474 | 508.6 | 3.07 | |
| | (3,4-difluoro-2- | | | | |
| | methoxyphenyl)-4,5- | | | | |
| | dimethyl-N-(2-(S- | | | | |
| | methylsulfonimidoyl) | | | | |
| | pyridin-4-yl)-5- | | | | |
| | (trifluoromethyl) | | | | |
| | tetrahydrofuran-2- | | | | |
| | carboxamide | | | | |
| | (second eluting isomer | | | | |
| | by SFC using AD-H | | | | |
| | column then achiral | | | | |
| | SFC purification using | | | | |
| | Lux Cellulose-2 | | | | |
| | column) | | | | |
| 55 | rel-(2R,3S,4S,5R)-3- | 507.474 | 508.6 | 3.07 | ¹H NMR (500 MHZ, |
| | (3,4-difluoro-2- | | | | Methanol-d₄) δ 8.47 (d, J = |
| | methoxyphenyl)-4,5- | | | | 5.5 Hz, 1H), 8.34 (d, J = 2.0 |
| | dimethyl-N-(2-(S- | | | | Hz, 1H), 7.78 (dd, J = 5.5, |
| | methylsulfonimidoyl) | | | | 2.1 Hz, 1H), 7.02 (ddd, J = |
| | pyridin-4-yl)-5- | | | | 8.2, 5.6, 2.1 Hz, 1H), 6.88 |
| | (trifluoromethyl) | | | | (td, J = 9.3, 7.6 Hz, 1H), 4.99 |
| | tetrahydrofuran-2- | | | | (d, J = 10.4 Hz, 1H), 4.23 |
| | carboxamide | | | | (dd, J = 10.3, 8.0 Hz, 1H), |
| | (third eluting isomer | | | | 3.90 (d, J = 2.3 Hz, 3H), 3.12 |
| | by SFC using AD-H | | | | (s, 3H), 2.70 (p, J = 7.7 Hz, |
| | column then achiral | | | | 1H), 1.56 (d, J = 1.4 Hz, 3H), |
| | SFC purification using | | | | 0.72 (dq, J = 7.4, 2.3 Hz, 3H) |
| | AS-H column) | | | | ppm. |
| 56 | rel-(2S,3R,4R,5S)-3- | 507.474 | 508.6 | 3.07 | ¹H NMR (500 MHZ, |
| | (3,4-difluoro-2- | | | | Methanol-d₄) δ 8.47 (d, J = |
| | methoxyphenyl)-4,5- | | | | 5.5 Hz, 1H), 8.34 (d, J = 2.0 |
| | dimethyl-N-(2-(S- | | | | Hz, 1H), 7.78 (dd, J = 5.5, |
| | methylsulfonimidoyl) | | | | 2.0 Hz, 1H), 7.02 (ddd, J = |
| | pyridin-4-yl)-5- | | | | 8.2, 5.6, 2.2 Hz, 1H), 6.88 |
| | (trifluoromethyl) | | | | (ddd, J = 10.0, 8.9, 7.5 Hz, |
| | tetrahydrofuran-2- | | | | 1H), 4.99 (d, J = 10.4 Hz, |
| | carboxamide | | | | 1H), 4.23 (dd, J = 10.3, 8.0 |
| | (forth eluting isomer | | | | Hz, 1H), 3.90 (d, J = 2.2 Hz, |
| | by SFC using AD-H | | | | 3H), 3.12 (s, 3H), 2.70 (p, J = |
| | column then achiral | | | | 7.6 Hz, 1H), 1.58-1.50 (m, |
| | SFC purification using | | | | 3H), 0.72 (dq, J = 7.4, 2.3 |
| | Lux Cellulose-2 | | | | Hz, 3H) ppm. |
| | column) | | | | |

The following compounds were made using the method described in Example 1, except that different coupling partners were used in the amide coupling step 11 and General Method I was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 57 | rel-(2R,3S,4S,5R)-3- | 491.427 | 492.9 | 2.81 | ¹H NMR (500 MHZ, |
| | (3,4-difluoro-2- | | | | Chloroform-d) δ 8.83 (s, 1H), |
| | methoxyphenyl)-N-(5- | | | | 8.39 (s, 1H), 8.28 (d, J = 6.2 |
| | fluoro-2- | | | | Hz, 1H), 7.09 (t, J = 7.1 Hz, |
| | ((methylamino)methyl) | | | | 1H), 6.91 (q, J = 8.7 Hz, 1H), |
| | pyridin-4-yl)-4,5- | | | | 5.04 (d, J = 11.0 Hz, 1H), |
| | dimethyl-5- | | | | 4.11-4.05 (m, 1H), 4.01 (d, |
| | (trifluoromethyl) | | | | J = 2.7 Hz, 3H), 3.77 (s, 2H), |
| | tetrahydrofuran-2- | | | | 2.75 (q, J = 7.6 Hz, 1H), 2.42 |
| | carboxamide | | | | (s, 3H), 1.68 (s, 3H), 0.90- |
| | (precursor was first | | | | 0.67 (m, 3H) ppm. |
| | eluting isomer by SFC | | | | |
| | using a Whelk-O1 | | | | |
| | column) | | | | |
| 58 | rel-(2S,3R,4R,5S)-3- | 491.427 | 492.2 | 2.83 | ¹H NMR (500 MHZ, |
| | (3,4-difluoro-2- | | | | Chloroform-d) δ 8.94 (s, 1H), |
| | methoxyphenyl)-N-(5- | | | | 8.41 (d, J = 1.7 Hz, 1H), 8.38 |
| | fluoro-2- | | | | (d, J = 5.7 Hz, 1H), 7.13- |
| | ((methylamino)methyl) | | | | 7.00 (m, 1H), 6.91 (q, J = 8.7 |
| | pyridin-4-yl)-4,5- | | | | Hz, 1H), 5.05 (d, J = 11.1 |
| | dimethyl-5- | | | | Hz, 1H), 4.29-4.15 (m, 2H), |
| | (trifluoromethyl) | | | | 4.08 (dd, J = 11.0, 7.9 Hz, |
| | tetrahydrofuran-2- | | | | 1H), 4.01 (d, J = 2.8 Hz, 3H), |
| | carboxamide | | | | 2.80 (s, 3H), 2.79-2.75 (m, |
| | (precursor was second | | | | 1H), 1.69 (s, 3H), 0.80 (dd, |
| | eluting isomer by SFC | | | | J = 7.7, 2.2 Hz, 3H) ppm. |
| | using a Whelk-O1 | | | | |
| | column) | | | | |
| 59 | rel-(2S,3R,4R,5S)-3- | 517.489 | | 3.26 | ¹H NMR (500 MHz, DMSO- |
| | (3,4-difluoro-2- | | | | d₆) δ 10.54 (s, 1H), 8.35 (d, |
| | methoxyphenyl)-N-(2- | | | | J = 5.5 Hz, 1H), 7.74-7.57 |
| | (((2- | | | | (m, 1H), 7.50 (dd, J = 5.6, |
| | methoxyethyl)amino) | | | | 2.1 Hz, 1H), 7.26-7.05 (m, |
| | methyl)pyridin-4-yl)- | | | | 2H), 5.09 (d, J = 10.2 Hz, |
| | 4,5-dimethyl-5- | | | | 1H), 4.25 (dd, J = 10.3, 7.7 |
| | (trifluoromethyl) | | | | Hz, 1H), 3.96 (d, J = 2.1 Hz, |
| | tetrahydrofuran-2- | | | | 3H), 3.74 (s, 2H), 3.40 (t, J = |
| | carboxamide | | | | 5.6 Hz, 2H), 3.23 (s, 3H), |
| | (precursor was first | | | | 2.78 (p, J = 7.5 Hz, 1H), 2.67 |
| | eluting isomer by SFC | | | | (t, J = 5.6 Hz, 2H), 1.60 (s, |
| | using a Whelk-O1 | | | | 3H), 0.78-0.69 (m, 3H) |
| | column) | | | | ppm. |
| 60 | rel-(2R,3S,4S,5R)-3- | 517.489 | 518.6 | 3.28 | ¹H NMR (500 MHZ, DMSO- |
| | (3,4-difluoro-2- | | | | d₆) δ 10.54 (s, 1H), 8.36 (d, |
| | methoxyphenyl)-N-(2- | | | | J = 5.5 Hz, 1H), 7.64 (d, J = |
| | (((2- | | | | 2.0 Hz, 1H), 7.50 (dd, J = |
| | methoxyethyl)amino) | | | | 5.6, 2.1 Hz, 1H), 7.21-7.11 |
| | methyl)pyridin-4-yl)- | | | | (m, 2H), 5.09 (d, J = 10.3 Hz, |
| | 4,5-dimethyl-5- | | | | 1H), 4.25 (dd, J = 10.3, 7.6 |
| | (trifluoromethyl) | | | | Hz, 1H), 3.96 (d, J = 2.1 Hz, |
| | tetrahydrofuran-2- | | | | 3H), 3.76 (s, 2H), 3.41 (t, J = |
| | carboxamide | | | | 5.6 Hz, 2H), 3.24 (s, 3H), |
| | (precursor was second | | | | 2.78 (p, J = 7.5 Hz, 1H), 2.69 |
| | eluting isomer by SFC | | | | (t, J = 5.6 Hz, 2H), 1.60 (s, |
| | using a Whelk-O1 | | | | 3H), 0.77-0.66 (m, 3H) |
| | column) | | | | ppm. |

The following compounds were made using the method described in Example 1, except that 2-[[tert-butyl(dimethyl)silyl]oxymethyl]pyridin-4-amine was used in the amide coupling step 11, described below, and General Method J was used as the final step:

To an ice-cooled solution of rac-(2R,3S,4S,5R)-3-(3,4-difluoro-2-methoxyphenyl)-4,5-dimethyl-5-(trifluoromethyl)tetrahydrofuran-2-carboxylic acid (150 mg, 0.3895 mmol) in 2-methyltetrahydrofuran (5 mL) was added DMF (45 μL of 0.86 M, 0.03870 mmol) as a solution in THF and carefully oxalyl chloride (70 μL, 0.8024 mmol). The mixture was stirred and warmed to ambient temperature over 30 minutes. The reaction mixture was concentrated in vacuo and the residue dissolved in 2-methyltetrahydrofuran (3 mL). This solution was added to an ice-cooled solution of 2-[[tert-butyl(dimethyl)silyl]oxymethyl]pyridin-4-amine (100 mg, 0.4195 mmol) and TEA (265 μL, 1.901 mmol) in 2-methyltetrahydrofuran (3 mL). The resulting mixture was stirred and warmed to ambient temperature over 2 hours. The reaction mixture was then quenched with water (10 mL) and the layers separated. The aqueous layer was extracted with EtOAc (2×10 mL) and the combined organics extracts were dried over MgSO₄, filtered and concentrated in vacuo. The residue was purified by column chromatography (12 g SiO₂, eluting with 0 to 30% EtOAc in heptane, loaded in DCM) to give rac-(2R,3S,4S,5R)—N-(2-(((tert-butyldimethylsilyl)oxy)methyl)pyridin-4-yl)-3-(3,4-difluoro-2-methoxyphenyl)-4,5-dimethyl-5-(trifluoromethyl)tetrahydrofuran-2-carboxamide (142.3 mg, 64%) as a colourless oil. ¹H NMR (500 MHz, Chloroform-d) δ 8.45 (s, 1H), 8.40 (d, J=5.6 Hz, 1H), 7.63 (d, J=5.5 Hz, 1H), 7.41 (s, 1H), 7.10-7.07 (m, 1H), 6.91 (td, J=9.3, 7.5 Hz, 1H), 5.00 (d, J=10.8 Hz, 1H), 4.81 (s, 2H), 4.11 (dd, J=10.7, 8.1 Hz, 1H), 4.00 (d, J=2.7 Hz, 3H), 2.75 (p, J=7.7 Hz, 1H), 1.67 (s, 3H), 0.96 (s, 9H), 0.81-0.79 (dd, J=7.5, 2.3 Hz, 3H), 0.13 (d, J=1.9 Hz, 6H) ppm; ¹⁹F NMR (471 MHz, Chloroform-d) δ−74.59, −137.08 (d, J=23.1 Hz), −154.52 (d, J=21.5 Hz) ppm; ESI-MS m/z calc. 574.22864, found 575.7 (M+1)⁺; 573.8 (M−1)⁻; Retention time: 1.23 minutes.

The enantiomers of rac-(2R,3S,4S,5R)—N-(2-(((tert-butyldimethylsilyl)oxy)methyl)pyridin-4-yl)-3-(3,4-difluoro-2-methoxyphenyl)-4,5-dimethyl-5-(trifluoromethyl)tetrahydrofuran-2-carboxamide (142 mg, 0.2471 mmol) were separated by chiral SFC using an (R,R)-Whelk-Olcolumn, 5 μm particle size, 25 cm×21.2 mm from Regis Technologies (Mobile phase: 30% acetonitrile:methanol (in a 1:1 ratio, supplemented with 0.2% DMIPA); System pressure: 100 bar) on a Minigram SFC instrument from Berger Instruments to give:

First eluting isomer (r.t.=2.19 minutes): rel-(2S,3R,4R,5S)-N-(2-(((tert-butyldimethylsilyl)oxy)methyl)pyridin-4-yl)-3-(3,4-difluoro-2-methoxyphenyl)-4,5-dimethyl-5-(trifluoromethyl)tetrahydrofuran-2-carboxamide (56.2 mg, 79%) as white solid. ¹H NMR (500 MHz, Chloroform-d) δ 8.46 (s, 1H), 8.40 (d, J=5.6 Hz, 1H), 7.63 (s, 1H), 7.42 (s, 1H), 7.10-7.07 (m, 1H), 6.93-6.88 (m, 1H), 5.01 (d, J=10.8 Hz, 1H), 4.82 (s, 2H), 4.11 (dd, J=10.8, 8.0 Hz, 1H), 4.00 (d, J=2.7 Hz, 3H), 2.75 (p, J=7.7 Hz, 1H), 1.67 (s, 3H), 0.96 (s, 9H), 0.81-0.79 (m, 3H), 0.13 (d, J=2.0 Hz, 6H) ppm. ¹⁹F NMR (471 MHz, Chloroform-d) δ−74.59, −137.27 (d, J=19.0 Hz), −154.51 (d, J=18.9 Hz) ppm. ESI-MS m/z calc. 574.22864, found 575.2 (M+1)⁺; 573.3 (M−1)⁻; Retention time: 4.27 minutes.

Second eluting isomer (r.t.=3.90 minutes): rel-(2R,3S,4S,5R)—N-(2-(((tert-butyldimethylsilyl)oxy)methyl)pyridin-4-yl)-3-(3,4-difluoro-2-methoxyphenyl)-4,5-dimethyl-5-(trifluoromethyl)tetrahydrofuran-2-carboxamide (52.5 mg, 74%) as white solid. ¹H NMR (500 MHz, Chloroform-d) δ 8.46 (s, 1H), 8.40 (d, J=5.7 Hz, 1H), 7.64 (s, 1H), 7.43 (s, 1H), 7.10-7.07 (m, 1H), 6.93-6.88 (m, 1H), 5.01 (d, J=10.7 Hz, 1H), 4.83 (s, 2H), 4.11 (dd, J=10.8, 8.0 Hz, 1H), 4.00 (d, J=2.7 Hz, 3H), 2.75 (p, J=7.8 Hz, 1H), 1.67 (s, 3H), 0.96 (s, 9H), 0.81-0.79 (m, 3H), 0.13 (d, J=1.8 Hz, 6H) ppm. ¹⁹F NMR (471 MHz, Chloroform-d) δ−74.59, −137.07, −154.50 ppm. ESI-MS m/z calc. 574.22864, found 575.2 (M+1)⁺; 573.3 (M−1)⁻; Retention time: 4.26 minutes.

In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 61 | rel-(2S,3R,4R,5S)-3- | 460.394 | 461.7 | 3.13 | ¹H NMR (500 MHZ, DMSO- |
| | (3,4-difluoro-2- | | | | d6) δ 10.58 (s, 1H), 8.33 (d, |
| | methoxyphenyl)-N-(2- | | | | J = 5.5 Hz, 1H), 7.72 (d, J = 2.0 |
| | (hydroxymethyl) | | | | Hz, 1H), 7.50 (dd, J = 5.5, 2.2 |
| | pyridin-4-yl)-4,5- | | | | Hz, 1H), 7.20-7.12 (m, 2H), |
| | dimethyl-5- | | | | 5.39 (s, 1H), 5.09 (d, J = 10.2 |
| | (trifluoromethyl) | | | | Hz, 1H), 4.50 (s, 2H), 4.25 |
| | tetrahydrofuran-2- | | | | (dd, J = 10.3, 7.7 Hz, 1H), 3.96 |
| | carboxamide | | | | (d, J = 2.0 Hz, 3H), 2.78 (p, J = |
| | (precursor was first | | | | 7.5 Hz, 1H), 1.60 (s, 3H), 0.74 |
| | eluting isomer by SFC | | | | (dd, J = 7.2, 2.5 Hz, 3H) ppm. |
| | using Whelk-O1 | | | | |
| | column) | | | | |
| 14 | rel-(2R,3S,4S,5R)-3- | 460.394 | 461.7 | 3.13 | ¹H NMR (500 MHZ, DMSO- |
| | (3,4-difluoro-2- | | | | d6) δ 10.58 (s, 1H), 8.33 (d, |
| | methoxyphenyl)-N-(2- | | | | J = 5.5 Hz, 1H), 7.71 (dd, J = |
| | (hydroxymethyl) | | | | 2.1, 0.8 Hz, 1H), 7.50 (dd, J = |
| | pyridin-4-yl)-4,5- | | | | 5.5, 2.2 Hz, 1H), 7.20-7.12 |
| | dimethyl-5- | | | | (m, 2H), 5.39 (s, 1H), 5.09 (d, |
| | (trifluoromethyl) | | | | J = 10.3 Hz, 1H), 4.50 (s, 2H), |
| | tetrahydrofuran-2- | | | | 4.25 (dd, J = 10.3, 7.6 Hz, 1H), |
| | carboxamide | | | | 3.96 (d, J = 2.1 Hz, 3H), 2.78 |
| | (precursor was second | | | | (p, J = 7.5 Hz, 1H), 1.60 (s, |
| | eluting isomer by SFC | | | | 3H), 0.74 (dd, J = 7.5, 2.4 Hz, |
| | using Whelk-O1 | | | | 3H) ppm. |
| | column) | | | | |

The following compound was made using the method described in Example 1, except that a different coupling partner was used in the amide coupling step 11. SFC step 12 was omitted and General Method D and then General Method J were used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 62 | rac-(2R,3S,4S,5R)-3- | 538.485 | 539.1 | 3.19 | ¹H NMR (400 MHZ, |
| | (3,4-difluoro-2- | | | | Chloroform-d) δ 8.93 (s, 1H), |
| | methoxyphenyl)-N- | | | | 8.60 (d, J = 5.5 Hz, 1H), 8.15- |
| | (2-((2- | | | | 8.04 (m, 2H), 7.11 (ddd, J = 8.2, |
| | hydroxyethyl) | | | | 5.6, 2.2 Hz, 1H), 6.93 (td, J = |
| | sulfonyl)pyridin-4-yl)- | | | | 9.2, 7.4 Hz, 1H), 5.09 (d, J = |
| | 4,5-dimethyl-5- | | | | 11.1 Hz, 1H), 4.13-4.10 (m, |
| | (trifluoromethyl) | | | | 2H), 4.03 (d, J = 2.9 Hz, 3H), |
| | tetrahydrofuran-2- | | | | 3.66-3.58 (m, 2H), 3.39-3.30 |
| | carboxamide | | | | (m, 1H), 2.79 (p, J = 7.7 Hz, |
| | | | | | 1H), 1.74-1.69 (m, 3H), 1.45 |
| | | | | | (p, J = 7.4 Hz, 1H), 0.82 (dq, |
| | | | | | J = 7.3, 2.4 Hz, 3H) ppm. |

### Example 2

rel-(2R,3S,4S,5R)-3-(2-(difluoromethoxy)-3,4-difluorophenyl)-4,5-dimethyl-N-(pyridazin-4-yl)-5-(trifluoromethyl)tetrahydrofuran-2-carboxamide (63) and rel-(2R,3S,4S,5R)-3-(2-(difluoromethoxy)-3,4-difluorophenyl)-4,5-dimethyl-N-(pyridazin-4-yl)-5-(trifluoromethyl)tetrahydrofuran-2-carboxamide (64)

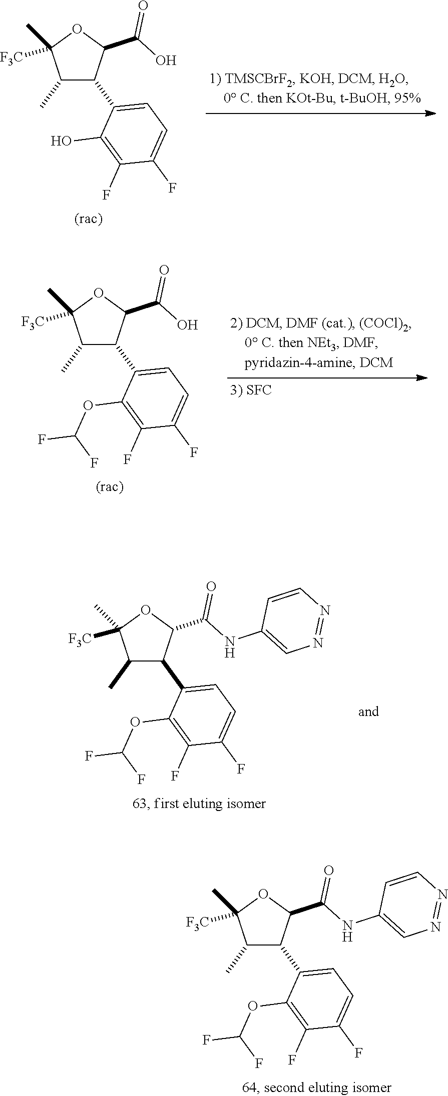 [SMILES: C[C@H]1[C@@H](c2ccc(F)c(F)c2O)[C@H](C(=O)O)O[C@@]1(C)C(F)(F)F ; C[C@H]1[C@@H](c2ccc(F)c(F)c2OC(F)F)[C@H](C(=O)O)O[C@@]1(C)C(F)(F)F ; C[C@@H]1[C@H](c2ccc(F)c(F)c2OC(F)F)[C@@H](C(=O)Nc2ccnnc2)O[C@]1(C)C(F)(F)F ; C[C@H]1[C@@H](c2ccc(F)c(F)c2OC(F)F)[C@H](C(=O)Nc2ccnnc2)O[C@@]1(C)C(F)(F)F]

### Step 1:

To a solution of rac-(2R,3S,4S,5R)-3-(3,4-difluoro-2-hydroxyphenyl)-4,5-dimethyl-5-(trifluoromethyl)tetrahydrofuran-2-carboxylic acid (9.30 g, 27.33 mmol) in DCM (50 mL) stirring at 0° C. was added a solution of KOH (18.4 g, 328.0 mmol) in H₂O (50 mL) and the solution was stirred vigorously. [Bromo(difluoro)methyl]-trimethyl-silane (22.5 g, 110.8 mmol) was added and stirring continued at this temperature. Upon complete consumption of the starting material, the mixture was acidified by addition of HCl 1N, extracted with DCM, and concentrated in vacuo. The resultant oil was dissolved in tert-butanol (50 mL) at ambient temperature and KOt-Bu (7.5 g, 66.84 mmol) was added. After complete conversion the mixture was acidified with 1N HCl, diluted with DCM, the layers separated, and the aqueous layer extracted. The organic phase was washed with water and concentrated in vacuo to give rac-(2R,3S,4S,5R)-3-(2-(difluoromethoxy)-3,4-difluorophenyl)-4,5-dimethyl-5-(trifluoromethyl)tetrahydrofuran-2-carboxylic acid (10.10 g, 95%) which was used without further purification.

### Step 2:

To an ice-cooled solution of rac-(2R,3S,4S,5R)-3-(2-(difluoromethoxy)-3,4-difluorophenyl)-4,5-dimethyl-5-(trifluoromethyl)tetrahydrofuran-2-carboxylic acid (10.10 g, 25.88 mmol) in DCM (100 mL) stirring at 0° C., was added DMF (400 μL, 5.17 mmol) and oxalyl chloride (4.85 mL, 55.60 mmol). The mixture was warmed to ambient temperature over 30 min before being concentrated in vacuo. The residue was dissolved in DCM (2 mL), cooled in an ice bath and TEA (49 μL, 0.3516 mmol) and pyridazin-4-amine (35.9 mg, 0.3775 mmol) were added sequentially. The reaction was stirred for 90 minutes, allowing to warm to ambient temperature, quenched with MeOH and concentrated in vacuo. Crude products were purified by flash chromatography (4 g SiO₂, 0 to 100% EtOAc in heptane) to give rac-(2R,3S,4S,5R)-3-(2-(difluoromethoxy)-3,4-difluorophenyl)-4,5-dimethyl-N-(pyridazin-4-yl)-5-(trifluoromethyl)tetrahydrofuran-2-carboxamide (79.5 mg, 66%). ESI-MS m/z calc. 467.108, found 468.6 (M+1)⁺; 466.7 (M−1)⁻; Retention time: 0.89 minutes.

### Step 3:

Purification of rac-(2R,3S,4S,5R)-3-(2-(difluoromethoxy)-3,4-difluorophenyl)-4,5-dimethyl-N-(pyridazin-4-yl)-5-(trifluoromethyl)tetrahydrofuran-2-carboxamide (50 mg, 0.1070 mmol) by chiral SFC [System: (R′R) Whelk 0-1 column, 3-5 μm particle size, 5.0 cm×3.0 mm from Regis Technologies with Solvent A: liquid CO₂; Solvent B: methanol with 20 mM NH₃ on a UPC2-SFC instrument from Waters Corp] gave:

First eluting isomer (retention time=4.28 mins): rel-(2S,3R,4R,5S)-3-(2-(difluoromethoxy)-3,4-difluorophenyl)-4,5-dimethyl-N-(pyridazin-4-yl)-5-(trifluoromethyl)tetrahydrofuran-2-carboxamide (63, 23.0 mg, 80%). ¹H NMR (500 MHz, Chloroform-d) δ 9.19 (dd, J=2.7, 1.0 Hz, 1H), 9.04 (dd, J=5.9, 0.9 Hz, 1H), 8.61 (s, 1H), 7.98 (dd, J=5.9, 2.8 Hz, 1H), 7.24-7.09 (m, 2H), 6.65 (d, J=73.7 Hz, 1H), 5.00 (d, J=11.1 Hz, 1H), 4.19 (dd, J=11.1, 8.2 Hz, 1H), 2.82 (p, J=7.8 Hz, 1H), 1.69 (d, J=1.2 Hz, 3H), 0.84 (dq, J=7.3, 2.3 Hz, 3H). ESI-MS m/z calc. 467.108, found 468.2 (M+1)⁺; 466.1 (M−1)⁻; Retention time: 3.17 minutes.

Second eluting isomer (retention time=7.52 mins): rel-(2R,3S,4S,5R)-3-(2-(difluoromethoxy)-3,4-difluorophenyl)-4,5-dimethyl-N-(pyridazin-4-yl)-5-(trifluoromethyl)tetrahydrofuran-2-carboxamide (64, 21.4 mg, 84%). ¹H NMR (500 MHz, Chloroform-d) δ 9.17 (dd, J=2.8, 1.0 Hz, 1H), 9.04 (dd, J=5.8, 0.9 Hz, 1H), 8.48 (s, 1H), 7.97 (dd, J=5.9, 2.8 Hz, 1H), 7.23-7.12 (m, 2H), 6.65 (ddd, J=74.6, 73.7, 0.9 Hz, 1H), 5.00 (d, J=11.1 Hz, 1H), 4.18 (dd, J=11.1, 8.3 Hz, 1H), 2.82 (p, J=7.7 Hz, 1H), 1.70 (d, J=1.1 Hz, 3H), 0.85 (dd, J=7.6, 2.3 Hz, 3H). ESI-MS m/z calc. 467.108, found 468.2 (M+1)⁺; 466.1 (M−1)⁻; Retention time: 3.17 minutes.

The following compounds were made using a method similar to that described in Example 2 except different amines were used as coupling partners in Step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 65 | rel-(2R,3S,4S,5R)-3- | 466.349 | 467.4 | 3.29 | ¹H NMR (500 MHZ, |
| | (2-(difluoromethoxy)- | | | | Chloroform-d) δ 8.60 (d, J = |
| | 3,4-difluorophenyl)- | | | | 2.6 Hz, 1H), 8.38 (dd, J = 4.7, |
| | 4,5-dimethyl-N- | | | | 1.4 Hz, 1H), 8.33 (s, 1H), 8.09 |
| | (pyridin-3-yl)-5- | | | | (ddd, J = 8.4, 2.7, 1.5 Hz, 1H), |
| | (trifluoromethyl) | | | | 7.29-7.22 (m, 2H), 7.14 (td, J |
| | tetrahydrofuran-2- | | | | J = 9.1, 7.5 Hz, 1H), 6.64 (t, J = |
| | carboxamide | | | | 74.5 Hz, 1H), 4.99 (d, J = 11.0 |
| | (first eluting isomer by | | | | Hz, 1H), 4.18 (dd, J = 11.0, 8.1 |
| | SFC using ChiralPak | | | | Hz, 1H), 2.81 (p, J = 7.7 Hz, |
| | IB column) | | | | 1H), 1.70 (d, J = 1.2 Hz, 3H), |
| | | | | | 0.88-0.80 (m, 3H) ppm. |
| 66 | rel-(2S,3R,4R,5S)-3- | 466.349 | 467.4 | 3.29 | ¹H NMR (500 MHZ, |
| | (2-(difluoromethoxy)- | | | | Chloroform-d) δ 8.60 (d, J = |
| | 3,4-difluorophenyl)- | | | | 2.6 Hz, 1H), 8.38 (dd, J = 4.8, |
| | 4,5-dimethyl-N- | | | | 1.5 Hz, 1H), 8.34 (s, 1H), 8.09 |
| | (pyridin-3-yl)-5- | | | | (ddd, J = 8.4, 2.7, 1.5 Hz, 1H), |
| | (trifluoromethyl) | | | | 7.29-7.21 (m, 2H), 7.14 (td, |
| | tetrahydrofuran-2- | | | | J = 9.1, 7.4 Hz, 1H), 6.64 (ddd, |
| | carboxamide | | | | J = 74.5, 73.7, 0.9 Hz, 1H), 4.99 |
| | (second eluting isomer | | | | (d, J = 11.0 Hz, 1H), 4.18 (dd, |
| | by SFC using | | | | J = 11.0, 8.1 Hz, 1H), 2.81 (p, |
| | ChiralPak IB column) | | | | J = 7.7 Hz, 1H), 1.70 (d, J = |
| | | | | | 1.2 Hz, 3H), 0.87-0.81 (m, |
| | | | | | 3H) ppm. |
| 67 | rel-(2S,3R,4R,5S)-3- | 496.375 | 497.1 | 3.08 | ¹H NMR (500 MHZ, DMSO- |
| | (2-(difluoromethoxy)- | | | | d₆) δ 9.93 (s, 1H), 8.10 (d, J = |
| | 3,4-difluorophenyl)- | | | | 2.8 Hz, 1H), 7.49-7.44 (m, |
| | 4,5-dimethyl-N-(1- | | | | 1H), |
| | methyl-6-oxo-1,6- | | | | 7.43-7.11 (m, 1H), 7.42 (dd, |
| | dihydropyridin-3-yl)- | | | | J = 9.7, 2.8 Hz, 1H), 7.33-7.29 |
| | 5-(trifluoromethyl) | | | | (m, 1H), 6.38 (d, J = 9.7 Hz, |
| | tetrahydrofuran-2- | | | | 1H), 5.06 (d, J = 10.4 Hz, 1H), |
| | carboxamide | | | | 4.23 (dd, J = 10.4, 7.5 Hz, 1H), |
| | (first eluting isomer by | | | | 3.39 (s, 3H), 2.73 (p, J = 7.5 |
| | SFC using Lux | | | | Hz, 1H), 1.58 (s, 3H), 0.75 (d, |
| | Cellulose-2 column) | | | | J = 6.3 Hz, 3H) ppm. |
| 68 | rel-(2R,3S,4S,5R)-3- | 496.375 | 497.1 | 3.08 | ¹H NMR (500 MHz, DMSO- |
| | (2-(difluoromethoxy)- | | | | d₆) δ 9.93 (s, 1H), 8.09 (d, J = |
| | 3,4-difluorophenyl)- | | | | 2.8 Hz, 1H), 7.49-7.44 (m, |
| | 4,5-dimethyl-N-(1- | | | | 1H), 7.43-7.11 (m, 1H), 7.42 |
| | methyl-6-oxo-1,6- | | | | (dd, J = 9.7, 2.9 Hz, 1H), 7.33- |
| | dihydropyridin-3-yl)- | | | | 7.29 (m, 1H), 6.38 (d, J = 9.7 |
| | 5-(trifluoromethyl) | | | | Hz, 1H), 5.06 (d, J = 10.4 Hz, |
| | tetrahydrofuran-2- | | | | 1H), 4.23 (dd, J = 10.4, 7.6 Hz, |
| | carboxamide | | | | 1H), 3.39 (s, 3H), 2.73 (p, J = |
| | (second eluting isomer | | | | 7.5 Hz, 1H), 1.58 (s, 3H), 0.75 |
| | by SFC using Lux | | | | (d, J = 6.7 Hz, 3H) ppm. |
| | Cellulose-2 column) | | | | |
| 69 | rel-(2S,3R,4R,5S)-N- | 491.359 | 492.1 | 3.44 | ¹H NMR (500 MHZ, |
| | (5-cyanopyridin-3-yl)- | | | | Methanol-d₄) δ 9.01 (d, J = 2.5 |
| | 3-(2- | | | | Hz, 1H), 8.66 (d, J = 1.7 Hz, |
| | (difluoromethoxy)-3,4- | | | | 1H), 8.53 (dd, J = 2.4, 1.8 Hz, |
| | difluorophenyl)-4,5- | | | | 1H), 7.36 (ddd, J = 9.1, 5.4, |
| | dimethyl-5- | | | | 2.0 Hz, 1H), 7.30 (td, J = 9.4, |
| | (trifluoromethyl) | | | | 7.4 Hz, 1H), 6.98 (td, J = 73.1, |
| | tetrahydrofuran-2- | | | | 1.2 Hz, 1H), 5.16 (d, J = 10.6 |
| | carboxamide | | | | Hz, 1H), 4.40 (dd, J = 10.5, 8.1 |
| | (first eluting isomer by | | | | Hz, 1H), 2.87 (p, J = 7.7 Hz, |
| | SFC using Whelk-O1 | | | | 1H), 1.72 (d, J = 1.1 Hz, 3H), |
| | column) | | | | 0.90 (dq, J = 7.4, 2.4 Hz, 3H) |
| | | | | | ppm. |
| 70 | rel-(2R,3S,4S,5R)-N- | 491.359 | 492.1 | 3.44 | ¹H NMR (500 MHZ, |
| | (5-cyanopyridin-3-yl)- | | | | Methanol-d₄) δ 9.01 (d, J = 2.5 |
| | 3-(2- | | | | Hz, 1H), 8.66 (d, J = 1.7 Hz, |
| | (difluoromethoxy)-3,4- | | | | 1H), 8.53 (dd, J = 2.5, 1.7 Hz, |
| | difluorophenyl)-4,5- | | | | 1H), 7.36 (ddd, J = 9.3, 5.4, |
| | dimethyl-5- | | | | 2.2 Hz, 1H), 7.30 (td, J = 9.4, |
| | (trifluoromethyl) | | | | 7.5 Hz, 1H), 6.97 (td, J = 73.1, |
| | tetrahydrofuran-2- | | | | 1.2 Hz, 1H), 5.16 (d, J = 10.6 |
| | carboxamide | | | | Hz, 1H), 4.39 (dd, J = 10.5, 8.1 |
| | (second eluting isomer | | | | Hz, 1H), 2.87 (p, J = 7.6 Hz, |
| | by SFC using Whelk- | | | | 1H), 1.72 (d, J = 1.1 Hz, 3H), |
| | O1 column) | | | | 0.90 (dq, J = 7.4, 2.4 Hz, 3H) |
| | | | | | ppm. |
| 71 | rel-(2S,3R,4R,5S)-3- | 544.44 | 545.1 | 3.41 | ¹H NMR (500 MHZ, |
| | (2-(difluoromethoxy)- | | | | Chloroform-d) δ 9.13 (s, 1H), |
| | 3,4-difluorophenyl)- | | | | 8.64 (dd, J = 1.6, 0.8 Hz, 1H), |
| | 4,5-dimethyl-N-(4- | | | | 8.60 (dd, J = 5.1, 0.8 Hz, 1H), |
| | (methylsulfonyl) | | | | 7.61 (dd, J = 5.1, 1.6 Hz, 1H), |
| | pyridin-2-yl)-5- | | | | 7.28-7.23 (m, 1H), 7.19 (td, |
| | (trifluoromethyl) | | | | J = 9.1, 7.4 Hz, 1H), 6.68 (ddd, |
| | tetrahydrofuran-2- | | | | J = 74.5, 73.8, 0.9 Hz, 1H), 5.04 |
| | carboxamide | | | | (d, J = 11.1 Hz, 1H), 4.21 (dd, |
| | (first eluting isomer by | | | | J = 11.1, 8.1 Hz, 1H), 3.08 (s, |
| | SFC using Whelk-O1 | | | | 3H), 2.90-2.74 (m, 1H), 1.73 |
| | column) | | | | (d, J = 1.2 Hz, 3H), 0.87 (dq, |
| | | | | | J = 7.4, 2.3 Hz, 3H) ppm. |
| 72 | rel-(2R,3S,4S,5R)-3- | 544.44 | 545.1 | 3.42 | ¹H NMR (500 MHZ, |
| | (2-(difluoromethoxy)- | | | | Chloroform-d) δ 8.99 (s, 1H), |
| | 3,4-difluorophenyl)- | | | | 8.50 (dd, J = 1.6, 0.8 Hz, 1H), |
| | 4,5-dimethyl-N-(4- | | | | 8.46 (dd, J = 5.1, 0.7 Hz, 1H), |
| | (methylsulfonyl) | | | | 7.47 (dd, J = 5.1, 1.6 Hz, 1H), |
| | pyridin-2-yl)-5- | | | | 7.12 (td, J = 5.2, 4.5, 2.1 Hz, |
| | (trifluoromethyl) | | | | 1H), 7.04 (td, J = 9.1, 7.4 Hz, |
| | tetrahydrofuran-2- | | | | 1H), 6.73-6.34 (m, 1H), 4.90 |
| | carboxamide | | | | (d, J = 11.1 Hz, 1H), 4.07 (dd, |
| | (second eluting isomer | | | | J = 11.1, 8.1 Hz, 1H), 2.94 (s, |
| | by SFC using Whelk- | | | | 3H), 2.70 (t, J = 7.7 Hz, 1H), |
| | O1 column) | | | | 1.59 (d, J = 1.2 Hz, 3H), 0.72 |
| | | | | | (dq, J = 7.4, 2.3 Hz, 3H) ppm. |
| 73 | rel-(2S,3R,4R,5S)-N- | 508.386 | 509.1 | 3.21 | ¹H NMR (400 MHZ, |
| | (4-carbamoylphenyl)- | | | | Chloroform-d) δ 8.48 (s, 1H), |
| | 3-(2- | | | | 7.86-7.77 (m, 2H), 7.70- |
| | (difluoromethoxy)-3,4- | | | | 7.58 (m, 2H), 7.27 (ddd, J = |
| | difluorophenyl)-4,5- | | | | 7.5, 6.3, 2.1 Hz, 1H), 7.16 (td, |
| | dimethyl-5- | | | | J = 9.2, 7.5 Hz, 1H), 6.67 (td, |
| | (trifluoromethyl) | | | | J = 74.2, 1.0 Hz, 1H), 6.17 (s, |
| | tetrahydrofuran-2- | | | | 1H), 5.89 (s, 1H), 5.01 (d, J = |
| | carboxamide | | | | 11.0 Hz, 1H), 4.20 (dd, J = |
| | (first eluting isomer by | | | | 11.0, 8.1 Hz, 1H), 2.83 (p, J = |
| | SFC using AS-H | | | | 7.6 Hz, 1H), 1.71 (d, J = 1.3 |
| | column) | | | | Hz, 3H), 0.86 (dt, J = 7.6, 2.3 |
| | | | | | Hz, 3H) ppm. |
| 74 | rel-(2R,3S,4S,5R)-N- | 508.386 | 509.1 | 3.22 | ¹H NMR (400 MHZ, |
| | (4-carbamoylphenyl)- | | | | Chloroform-d) δ 8.48 (s, 1H), |
| | 3-(2- | | | | 7.86-7.77 (m, 2H), 7.70- |
| | (difluoromethoxy)-3,4- | | | | 7.58 (m, 2H), 7.27 (ddd, J = |
| | difluorophenyl)-4,5- | | | | 7.5, 6.3, 2.1 Hz, 1H), 7.16 (td, |
| | dimethyl-5- | | | | J = 9.2, 7.5 Hz, 1H), 6.67 (td, |
| | (trifluoromethyl) | | | | J = 74.2, 1.0 Hz, 1H), 6.17 (s, |
| | tetrahydrofuran-2- | | | | 1H), 5.89 (s, 1H), 5.01 (d, J = |
| | carboxamide | | | | 11.0 Hz, 1H), 4.20 (dd, J = |
| | (second eluting isomer | | | | 11.0, 8.1 Hz, 1H), 2.83 (p, J = |
| | by SFC using AS-H | | | | 7.6 Hz, 1H), 1.71 (d, J = 1.3 |
| | column) | | | | Hz, 3H), 0.86 (dt, J = 7.6, 2.3 |
| | | | | | Hz, 3H) ppm. |
| 75 | rel-(2S,3R,4R,5S)-3- | 513.406 | 514.1 | 3.09 | ¹H NMR (400 MHZ, DMSO- |
| | (2-(difluoromethoxy)- | | | | d₆) δ 9.68 (s, 1H), 7.86 (s, 1H), |
| | 3,4-difluorophenyl)-N- | | | | 7.56-7.43 (m, 1H), 7.46- |
| | (1-(2-hydroxyethyl)-3- | | | | 7.07 (m, 2H), 5.17 (d, J = 10.4 |
| | methyl-1H-pyrazol-4- | | | | Hz, 1H), 4.79 (t, J = 5.3 Hz, |
| | yl)-4,5-dimethyl-5- | | | | 1H), 4.25 (dd, J = 10.5, 7.4 Hz, |
| | (trifluoromethyl) | | | | 1H), 3.97 (t, J = 5.6 Hz, 2H), |
| | tetrahydrofuran-2- | | | | 3.64 (q, J = 5.6 Hz, 2H), 2.74 |
| | carboxamide | | | | (p, J = 7.4 Hz, 1H), 2.06 (s, |
| | (first eluting isomer by | | | | 3H), 1.57 (s, 3H), 0.90-0.68 |
| | SFC using Lux | | | | (m, 3H) ppm. |
| | Cellulose-2 column) | | | | |
| 76 | rel-(2R,3S,4S,5R)-3- | 513.406 | 514.1 | 3.09 | ¹H NMR (400 MHZ, DMSO- |
| | (2-(difluoromethoxy)- | | | | d₆) δ 9.68 (s, 1H), 7.86 (s, 1H), |
| | 3,4-difluorophenyl)-N- | | | | 7.55-7.45 (m, 1H), 7.46- |
| | (1-(2-hydroxyethyl)-3- | | | | 7.08 (m, 2H), 5.17 (d, J = 10.4 |
| | methyl-1H-pyrazol-4- | | | | Hz, 1H), 4.79 (t, J = 5.3 Hz, |
| | yl)-4,5-dimethyl-5- | | | | 1H), 4.25 (dd, J = 10.5, 7.4 Hz, |
| | (trifluoromethyl) | | | | 1H), 3.97 (t, J = 5.6 Hz, 2H), |
| | tetrahydrofuran-2- | | | | 3.64 (q, J = 5.5 Hz, 2H), 2.74 |
| | carboxamide | | | | (t, J = 7.4 Hz, 1H), 2.06 (s, |
| | (second eluting isomer | | | | 3H), 1.57 (s, 3H), 0.81-0.68 |
| | by SFC using Lux | | | | (m, 3H) ppm. |
| | Cellulose-2 column) | | | | |
| 77 | rel-(2S,3R,4R,5S)-3- | 496.375 | 497.1 | 3.1 | ¹H NMR (500 MHz, |
| | (2-(difluoromethoxy)- | | | | Methanol-d₄) δ 7.58 (d, J = 7.5 |
| | 3,4-difluorophenyl)- | | | | Hz, 1H), 7.29-7.20 (m, 2H), |
| | 4,5-dimethyl-N-(1- | | | | 7.14-6.76 (m, 2H), 6.71 (dd, |
| | methyl-2-oxo-1,2- | | | | J = 7.4, 2.4 Hz, 1H), 5.06 (d, |
| | dihydropyridin-4-yl)- | | | | J = 10.2 Hz, 1H), 4.36 (dd, J = |
| | 5-(trifluoromethyl) | | | | 10.3, 8.1 Hz, 1H), 3.52 (s, 3H), |
| | tetrahydrofuran-2- | | | | 2.83 (p, J = 7.7 Hz, 1H), 1.66 |
| | carboxamide | | | | (d, J = 1.2 Hz, 3H), 0.86 (dt, |
| | (first eluting isomer by | | | | J = 7.3, 2.4 Hz, 3H) ppm. |
| | SFC using Whelk-O1 | | | | |
| | column) | | | | |
| 78 | rel-(2R,3S,4S,5R)-3- | 496.375 | 497.1 | 3.1 | ¹H NMR (500 MHZ, |
| | (2-(difluoromethoxy)- | | | | Methanol-d₄) δ 7.58 (d, J = 7.5 |
| | 3,4-difluorophenyl)- | | | | Hz, 1H), 7.30-7.22 (m, 2H), |
| | 4,5-dimethyl-N-(1- | | | | 7.13-6.76 (m, 2H), 6.71 (dd, |
| | methyl-2-oxo-1,2- | | | | J = 7.5, 2.4 Hz, 1H), 5.06 (d, J = |
| | dihydropyridin-4-yl)- | | | | 10.3 Hz, 1H), 4.36 (dd, J = |
| | 5-(trifluoromethyl) | | | | 10.3, 8.1 Hz, 1H), 3.52 (s, 3H), |
| | tetrahydrofuran-2- | | | | 2.88-2.77 (m, 1H), 1.66 (d, |
| | carboxamide | | | | J = 1.1 Hz, 3H), 0.86 (dt, J = |
| | (second eluting isomer | | | | 7.3, 2.4 Hz, 3H) ppm. |
| | by SFC using Whelk- | | | | |
| | O1 column) | | | | |
| 79 | rel-(2S,3R,4R,5S)-3- | 507.362 | 508 | 3.32 | ¹H NMR (500 MHZ, |
| | (2-(difluoromethoxy)- | | | | Methanol-d₄) δ 9.01 (dd, J = |
| | 3,4-difluorophenyl)- | | | | 7.5, 0.9 Hz, 1H), 8.51 (dd, J = |
| | 4,5-dimethyl-N- | | | | 2.1, 0.9 Hz, 1H), 7.56 (dd, J = |
| | (tetrazolo[1,5- | | | | 7.4, 2.1 Hz, 1H), 7.43-7.19 |
| | a]pyridin-7-yl)-5- | | | | (m, 2H), 6.96 (td, J = 73.1, 1.1 |
| | (trifluoromethyl) | | | | Hz, 1H), 5.16 (d, J = 10.4 Hz, |
| | tetrahydrofuran-2- | | | | 1H), 4.42 (dd, J = 10.4, 8.1 Hz, |
| | carboxamide | | | | 1H), 2.86 (p, J = 7.6 Hz, 1H), |
| | (first eluting isomer by | | | | 1.70 (d, J = 1.2 Hz, 3H), 0.89 |
| | SFC using Whelk-O1 | | | | (dq, J = 7.5, 2.3 Hz, 3H) ppm. |
| | column) | | | | |
| 80 | rel-(2R,3S,4S,5R)-3- | 507.362 | 508 | 3.31 | ¹H NMR (500 MHZ, |
| | (2-(difluoromethoxy)- | | | | Methanol-d₄) δ 9.01 (dd, J = |
| | 3,4-difluorophenyl)- | | | | 7.4, 0.9 Hz, 1H), 8.52 (dd, J = |
| | 4,5-dimethyl-N- | | | | 2.1, 0.9 Hz, 1H), 7.57 (dd, J = |
| | (tetrazolo[1,5- | | | | 7.5, 2.1 Hz, 1H), 7.42-7.18 |
| | a]pyridin-7-yl)-5- | | | | (m, 2H), 6.96 (td, J = 73.1, 1.1 |
| | (trifluoromethyl) | | | | Hz, 1H), 5.16 (d, J = 10.3 Hz, |
| | tetrahydrofuran-2- | | | | 1H), 4.42 (dd, J = 10.4, 8.1 Hz, |
| | carboxamide | | | | 1H), 2.86 (p, J = 7.6 Hz, 1H), |
| | (second eluting isomer | | | | 1.70 (d, J = 1.1 Hz, 3H), 0.89 |
| | by SFC using Whelk- | | | | (dq, J = 7.4, 2.3 Hz, 3H) ppm. |
| | O1 column) | | | | |

The following compounds were made using a method similar to that described in Example 2, except 2-(2,2-dimethyl-1,3-dioxolan-4-yl)pyridin-4-amine (second eluting isomer by SFC) was used as coupling partner in Step 2 and General Method B was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 81 | rel-(2S,3R,4R,5S)-3- | 526.401 | 527.5 | 3.08 | ¹H NMR (500 MHZ, |
| | (2- | | | | Chloroform-d) δ 8.56 (s, 1H), |
| | (difluoromethoxy)- | | | | 8.44 (d, J = 5.6 Hz, 1H), 7.58 (d, |
| | 3,4-difluorophenyl)- | | | | J = 2.1 Hz, 1H), 7.48 (dd, J = |
| | N-(2-(1,2- | | | | 5.8, 2.1 Hz, 1H), 7.24 (td, J = |
| | dihydroxyethyl) | | | | 6.1, 5.3, 2.8 Hz, 1H), 7.17 (dd, |
| | pyridin-4-yl)-4,5- | | | | J = 9.1, 7.4 Hz, 1H), 6.87-6.48 |
| | dimethyl-5- | | | | (m, 1H), 5.00 (d, J = 11.1 Hz, |
| | (trifluoromethyl) | | | | 1H), 4.79 (t, J = 4.7 Hz, 1H), |
| | tetrahydrofuran-2- | | | | 4.18 (dd, J = 11.1, 8.1 Hz, 1H), |
| | carboxamide | | | | 3.94 (dd, J = 11.3, 3.9 Hz, 1H), |
| | (first eluting isomer | | | | 3.78 (dd, J = 11.3, 5.4 Hz, 1H), |
| | by SFC using | | | | 2.83 (p, J = 7.6 Hz, 1H), 1.70 (d, |
| | Whelk-O1 column) | | | | J = 1.2 Hz, 3H), 0.85 (dq, J = |
| | | | | | 7.4, 2.3 Hz, 3H) ppm. |
| 82 | rel-(2R,3S,4S,5R)-3- | 526.401 | 527.5 | 3.08 | ¹H NMR (500 MHZ, |
| | (2- | | | | Chloroform-d) δ 8.70 (s, 1H), |
| | (difluoromethoxy)- | | | | 8.46 (d, J = 5.9 Hz, 1H), 7.63 (s, |
| | 3,4-difluorophenyl)- | | | | 1H), 7.54 (d, J = 5.7 Hz, 1H), |
| | N-(2-(1,2- | | | | 7.27-7.21 (m, 1H), 7.17 (td, J = |
| | dihydroxyethyl) | | | | 9.1, 7.4 Hz, 1H), 6.84-6.49 (m, |
| | pyridin-4-yl)-4,5- | | | | 1H), 5.02 (d, J = 11.0 Hz, 1H), |
| | dimethyl-5- | | | | 4.83 (t, J = 4.6 Hz, 1H), 4.20 |
| | (trifluoromethyl) | | | | (dd, J = 11.1, 8.1 Hz, 1H), 3.96 |
| | tetrahydrofuran-2- | | | | (dd, J = 11.4, 3.9 Hz, 1H), 3.80 |
| | carboxamide | | | | (dd, J = 11.4, 5.3 Hz, 1H), 2.83 |
| | (second eluting | | | | (p, J = 7.7 Hz, 1H), 1.71 (d, J = |
| | isomer by SFC using | | | | 1.2 Hz, 3H), 0.86 (dt, J = 7.5, |
| | Whelk-O1 column) | | | | 2.3 Hz, 3H) ppm. |

The following compounds were made using a method similar to that described in Example 2, except 2-(2-((trimethylsilyl)oxy)propan-2-yl)pyridin-4-amine was used as the coupling partner in Step 2 and TMS deprotection using conditions similar to General Method N, with 1 M HCl in THF, was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 83 | rel-(2S,3R,4R,5S)-3- | 524.429 | 525.7 | 3.40 | ¹H NMR (500 MHZ, Methanol- |
| | (2- | | | | d4) δ 8.40 (d, J = 5.7 Hz, 1H), |
| | (difluoromethoxy)- | | | | 7.98 (d, J = 2.2 Hz, 1H), 7.69 |
| | 3,4-difluorophenyl)- | | | | (dd, J = 5.8, 2.1 Hz, 1H), 7.38- |
| | N-(2-(2- | | | | 7.25 (m, 2H), 6.97 (td, J = 73.1, |
| | hydroxypropan-2- | | | | 1.2 Hz, 1H), 5.14 (d, J = 10.4 |
| | yl)pyridin-4-yl)-4,5- | | | | Hz, 1H), 4.41 (dd, J = 10.4, 8.0 |
| | dimethyl-5- | | | | Hz, 1H), 2.87 (p, J = 7.6 Hz, |
| | (trifluoromethyl) | | | | 1H), 1.70 (d, J = 1.1 Hz, 3H), |
| | tetrahydrofuran-2- | | | | 1.56 (s, 6H), 0.90 (dq, J = 7.6, |
| | carboxamide | | | | 2.4 Hz, 3H) ppm. |
| | (precursor was first | | | | |
| | eluting isomer by | | | | |
| | SFC using a Whelk- | | | | |
| | O1 column) | | | | |
| 84 | rel-(2R,3S,4S,5R)-3- | 524.429 | 525.7 | 3.40 | ¹H NMR (500 MHZ, Methanol- |
| | (2- | | | | d4) δ 8.40 (d, J = 5.7 Hz, 1H), |
| | (difluoromethoxy)- | | | | 7.98 (d, J = 2.2 Hz, 1H), 7.69 |
| | 3,4-difluorophenyl)- | | | | (dd, J = 5.8, 2.1 Hz, 1H), 7.38- |
| | N-(2-(2- | | | | 7.25 (m, 2H), 6.97 (td, J = 73.1, |
| | hydroxypropan-2- | | | | 1.2 Hz, 1H), 5.14 (d, J = 10.4 |
| | yl)pyridin-4-yl)-4,5- | | | | Hz, 1H), 4.41 (dd, J = 10.4, 8.0 |
| | dimethyl-5- | | | | Hz, 1H), 2.87 (p, J = 7.6 Hz, |
| | (trifluoromethyl) | | | | 1H), 1.70 (d, J = 1.1 Hz, 3H), |
| | tetrahydrofuran-2- | | | | 1.56 (s, 6H), 0.90 (dq, J = 7.6, |
| | carboxamide | | | | 2.4 Hz, 3H) ppm. |
| | (precursor was | | | | |
| | second eluting | | | | |
| | isomer by SFC using | | | | |
| | a Whelk-O1 column) | | | | |

The following compounds were made using a method similar to that described in Example 2, except different amines were used as coupling partners in Step 2 and using General Method D as the penultimate step prior to SFC separation. In the Table below, “MS r.t.” stands for Mass Spec retention time.

| | | LC/MS | Found | MS | |
|---|---|---|---|---|---|
| | Compound Name | (m/z calc.) | M + 1 | r.t. | NMR (shifts in ppm) |
| 85 | rel-(2R,3S,4S,5R)-3- | 544.44 | 545.05 | 3.36 | ¹H NMR (500 MHz, Methanol- |
| | (2- | | | | d₄) δ 8.59 (dd, J = 5.5, 0.6 Hz, |
| | (difluoromethoxy)- | | | | 1H), 7.95 (dd, J = 5.5, 2.1 Hz, |
| | 3,4-difluorophenyl)- | | | | 1H), 7.33-7.23 (m, 2H), 6.94 |
| | 4,5-dimethyl-N-(2- | | | | (td, J = 73.1, 1.0 Hz, 1H), 5.12 |
| | (methylsulfonyl) | | | | (d, J = 10.3 Hz, 1H), 4.38 (dd, |
| | pyridin-4-yl)-5- | | | | J = 10.4, 8.1 Hz, 1H), 3.21 (s, |
| | (trifluoromethyl) | | | | 3H), 2.93-2.77 (m, 1H), 1.76- |
| | tetrahydrofuran-2- | | | | 1.61 (m, 3H), 0.87 (dq, J = 7.4, |
| | carboxamide | | | | 2.3 Hz, 3H) ppm. |
| | (first eluting isomer | | | | |
| | by SFC using a | | | | |
| | Whelk-O1 column) | | | | |
| 86 | rel-(2S,3R,4R,5S)-3- | 544.44 | 545.3 | 3.38 | ¹H NMR (500 MHz, Methanol- |
| | (2- | | | | d₄) δ 8.60 (dd, J = 5.5, 0.6 Hz, |
| | (difluoromethoxy)- | | | | 1H), 8.41 (dd, J = 2.1, 0.6 Hz, |
| | 3,4-difluorophenyl)- | | | | 1H), 7.96 (dd, J = 5.5, 2.1 Hz, |
| | 4,5-dimethyl-N-(2- | | | | 1H), 7.37-7.22 (m, 2H), 6.95 |
| | (methylsulfonyl) | | | | (td, J = 73.1, 1.1 Hz, 1H), 5.13 |
| | pyridin-4-yl)-5- | | | | (d, J = 10.3 Hz, 1H), 4.39 (dd, |
| | (trifluoromethyl) | | | | J = 10.3, 8.1 Hz, 1H), 3.22 (s, |
| | tetrahydrofuran-2- | | | | 3H), 2.93-2.78 (m, 1H), 1.69 |
| | carboxamide | | | | (d, J = 1.1 Hz, 3H), 0.88 (dt, J = |
| | (second eluting | | | | 7.6, 2.3 Hz, 3H) ppm. |
| | isomer by SFC using | | | | |
| | a Whelk-O1 column) | | | | |
| 87 | rel-3-((2S,3R,4R,5S)- | 560.439 | 561.1 | 3.08 | ¹H NMR (500 MHz, Methanol- |
| | 3-(2- | | | | d₄) δ 9.12 (t, J = 1.7 Hz, 1H), |
| | (difluoromethoxy)- | | | | 8.58 (t, J = 1.5 Hz, 1H), 8.30 (t, |
| | 3,4-difluorophenyl)- | | | | J = 1.6 Hz, 1H), 7.40-7.24 (m, |
| | 4,5-dimethyl-5- | | | | 2H), 6.98 (td, J = 73.1, 1.2 Hz, |
| | (trifluoromethyl) | | | | 1H), 5.17 (d, J = 10.4 Hz, 1H), |
| | tetrahydrofuran-2- | | | | 4.38 (dd, J = 10.5, 8.1 Hz, 1H), |
| | carboxamido)-5- | | | | 3.28 (s, 3H), 2.87 (p, J = 7.6 Hz, |
| | (methylsulfonyl) | | | | 1H), 1.71 (d, J = 1.1 Hz, 3H), |
| | pyridine 1-oxide | | | | 0.90 (dq, J = 7.4, 2.4 Hz, 3H) |
| | (first eluting isomer | | | | ppm. |
| | by SFC using a | | | | |
| | Whelk-O1 column) | | | | |
| 88 | rel-3-((2R,3S,4S,5R)- | 560.439 | 561 | 3.07 | ¹H NMR (500 MHZ, Methanol- |
| | 3-(2- | | | | d₄) δ 9.12 (t, J = 1.7 Hz, 1H), |
| | (difluoromethoxy)- | | | | 8.58 (t, J = 1.6 Hz, 1H), 8.31 (t, |
| | 3,4-difluorophenyl)- | | | | J = 1.6 Hz, 1H), 7.39-7.25 (m, |
| | 4,5-dimethyl-5- | | | | 2H), 6.98 (td, J = 73.1, 1.2 Hz, |
| | (trifluoromethyl) | | | | 1H), 5.17 (d, J = 10.4 Hz, 1H), |
| | tetrahydrofuran-2- | | | | 4.38 (dd, J = 10.6, 8.0 Hz, 1H), |
| | carboxamido)-5- | | | | 3.28 (s, 3H), 2.87 (p, J = 7.7 Hz, |
| | (methylsulfonyl) | | | | 1H), 1.77-1.66 (m, 3H), 0.90 |
| | pyridine 1-oxide | | | | (dq, J = 7.4, 2.4 Hz, 3H) ppm. |
| | (second eluting | | | | |
| | isomer by SFC using | | | | |
| | a Whelk-O1 column) | | | | |
| 89 | rel-(2S,3R,4R,5S)-3- | 558.466 | 559 | 3.45 | ¹H NMR (500 MHZ, |
| | (2- | | | | Chloroform-d) δ 8.65 (s, 1H), |
| | (difluoromethoxy)- | | | | 8.60 (d, J = 5.4 Hz, 1H), 8.01 |
| | 3,4-difluorophenyl)- | | | | (dd, J = 5.5, 2.1 Hz, 1H), 7.98 |
| | N-(2- | | | | (d, J = 2.0 Hz, 1H), 7.22 (ddd, |
| | (ethylsulfonyl) | | | | J = 9.1, 5.3, 2.0 Hz, 1H), 7.16 (td, |
| | pyridin-4-yl)-4,5- | | | | J = 9.1, 7.3 Hz, 1H), 6.65 (ddd, |
| | dimethyl-5- | | | | J = 74.5, 73.7, 0.8 Hz, 1H), 4.99 |
| | (trifluoromethyl) | | | | (d, J = 11.2 Hz, 1H), 4.16 (dd, |
| | tetrahydrofuran-2- | | | | J = 11.1, 8.2 Hz, 1H), 3.42 (q, J = |
| | carboxamide | | | | 7.5 Hz, 2H), 2.82 (p, J = 7.7 Hz, |
| | (first eluting isomer | | | | 1H), 1.71 (d, J = 1.3 Hz, 3H), |
| | by SFC using a | | | | 1.30 (t, J = 7.5 Hz, 3H), 0.84 |
| | Whelk-O1 column) | | | | (dq, J = 7.5, 2.3 Hz, 3H) ppm. |
| 90 | rel-(2R,3S,4S,5R)-3- | 558.466 | 559 | 3.45 | ¹H NMR (500 MHZ, |
| | (2- | | | | Chloroform-d) δ 8.66 (s, 1H), |
| | (difluoromethoxy)- | | | | 8.60 (d, J = 5.5 Hz, 1H), 8.01 |
| | 3,4-difluorophenyl)- | | | | (dd, J = 5.5, 2.2 Hz, 1H), 7.98 |
| | N-(2- | | | | (dd, J = 2.2, 0.6 Hz, 1H), 7.21 |
| | (ethylsulfonyl) | | | | (ddd, J = 9.1, 5.3, 2.0 Hz, 1H), |
| | pyridin-4-yl)-4,5- | | | | 7.15 (td, J = 9.1, 7.3 Hz, 1H), |
| | dimethyl-5- | | | | 6.65 (ddd, J = 74.5, 73.7, 0.9 |
| | (trifluoromethyl) | | | | Hz, 1H), 4.99 (d, J = 11.1 Hz, |
| | tetrahydrofuran-2- | | | | 1H), 4.17 (dd, J = 11.1, 8.2 Hz, |
| | carboxamide | | | | 1H), 3.41 (q, J = 7.4 Hz, 2H), |
| | (second eluting | | | | 2.82 (p, J = 7.7 Hz, 1H), 1.70 (d, |
| | isomer by SFC using | | | | J = 1.2 Hz, 3H), 1.29 (t, J = 7.5 |
| | a Whelk-O1 column) | | | | Hz, 3H), 0.84 (dq, J = 7.4, 2.3 |
| | | | | | Hz, 3H) ppm. |

The following compounds were made using a method similar to that described in Example 2 except 2-(((tert-butyldimethylsilyl)oxy)methyl)pyridin-4-amine was used as the coupling partner in Step 2 and General Method J was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 91 | rel-(2S,3R,4R,5S)-3- | 496.375 | 497.1 | 3.19 | ¹H NMR (500 MHZ, |
| | (2- | | | | Chloroform-d) δ 8.60 (s, 1H), |
| | (difluoromethoxy)- | | | | 8.36 (d, J = 5.6 Hz, 1H), 7.51- |
| | 3,4-difluorophenyl)- | | | | 7.42 (m, 1H), 7.35 (dd, J = 5.8, |
| | N-(2- | | | | 2.0 Hz, 1H), 7.19-7.14 (m, |
| | (hydroxymethyl) | | | | 1H), 7.08 (td, J = 9.1, 7.5 Hz, |
| | pyridin-4-yl)-4,5- | | | | 1H), 6.83-6.33 (m, 1H), 4.93 |
| | dimethyl-5- | | | | (d, J = 11.0 Hz, 1H), 4.65 (s, |
| | (trifluoromethyl) | | | | 2H), 4.11 (dd, J = 11.0, 8.0 Hz, |
| | tetrahydrofuran-2- | | | | 1H), 3.22 (s, 1H), 2.74 (p, J = |
| | carboxamide | | | | 7.6 Hz, 1H), 1.62 (d, J = 1.2 Hz, |
| | (precursor was first | | | | 3H), 0.76 (dt, J = 7.5, 2.3 Hz, |
| | eluting isomer by | | | | 3H) ppm. |
| | SFC using a Whelk- | | | | |
| | O1 column) | | | | |
| 92 | rel-(2R,3S,4S,5R)-3- | 496.375 | 497.6 | 3.91 | ¹H NMR (500 MHZ, |
| | (2- | | | | Chloroform-d) δ 8.65 (s, 1H), |
| | (difluoromethoxy)- | | | | 8.47 (d, J = 5.7 Hz, 1H), 7.58 (d, |
| | 3,4-difluorophenyl)- | | | | J = 2.0 Hz, 1H), 7.50-7.39 (m, |
| | N-(2- | | | | 1H), 7.28-7.22 (m, 1H), 7.17 |
| | (hydroxymethyl) | | | | (td, J = 9.1, 7.4 Hz, 1H), 6.86- |
| | pyridin-4-yl)-4,5- | | | | 6.42 (m, 1H), 5.03 (d, J = 11.1 |
| | dimethyl-5- | | | | Hz, 1H), 4.76 (s, 2H), 4.19 (dd, |
| | (trifluoromethyl) | | | | J = 11.1, 8.0 Hz, 1H), 3.39- |
| | tetrahydrofuran-2- | | | | 3.26 (m, 1H), 2.83 (p, J = 7.7 |
| | carboxamide | | | | Hz, 1H), 1.71 (d, J = 1.2 Hz, |
| | (precursor was | | | | 3H), 0.86 (dt, J = 7.4, 2.4 Hz, |
| | second eluting | | | | 3H) ppm. |
| | isomer by SFC using | | | | |
| | a Whelk-O1 column) | | | | |

The following compounds were made using a method similar to that described in Example 2 except that methyl 5-aminopicolinate was used as the coupling partner in step 2 and General Method L was used as the penultimate step prior to SFC separation. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 93 | rel-5-((2S,3R,4R,5S)- | 509.374 | 510.1 | 3.2 | ¹H NMR (500 MHz, |
| | 3-(2- | | | | Methanol-d₄) δ 8.88 (dd, J = |
| | (difluoromethoxy)- | | | | 2.4, 0.9 Hz, 1H), 8.22 (dd, J = |
| | 3,4-difluorophenyl)- | | | | 8.6, 2.4 Hz, 1H), 8.09 (dd, J = |
| | 4,5-dimethyl-5- | | | | 8.5, 0.8 Hz, 1H), 7.41-7.25 |
| | (trifluoromethyl)tetra- | | | | (m, 1H), 6.97 (td, J = 73.1, 1.1 |
| | hydrofuran-2- | | | | Hz, 1H), 5.16 (d, J = 10.6 Hz, |
| | carboxamido)picolin- | | | | 1H), 4.41 (dd, J = 10.5, 8.1 Hz, |
| | amide | | | | 1H), 2.87 (p, J = 7.6 Hz, 3H), |
| | (first eluting isomer | | | | 0.90 (dq, J = 7.4, 2.4 Hz, 3H) |
| | by SFC using a | | | | ppm. |
| | Whelk-O1 column) | | | | |
| 94 | rel-5-((2R,3S,4S,5R)- | 509.374 | 510.1 | 3.2 | ¹H NMR (500 MHz, |
| | 3-(2- | | | | Methanol-d₄) δ 8.88 (dd, J = |
| | (difluoromethoxy)- | | | | 2.5, 0.8 Hz, 1H), 8.22 (dd, J = |
| | 3,4-difluorophenyl)- | | | | 8.6, 2.4 Hz, 1H), 8.09 (dd, J = |
| | 4,5-dimethyl-5- | | | | 8.7, 0.8 Hz, 1H), 7.42-7.24 |
| | (trifluoromethyl)tetra- | | | | (m, 1H), 6.97 (td, J = 73.1, 1.1 |
| | hydrofuran-2- | | | | Hz, 1H), 5.16 (d, J = 10.6 Hz, |
| | carboxamido)picolin- | | | | 1H), 4.41 (dd, J = 10.6, 8.0 Hz, |
| | amide | | | | 1H), 2.87 (p, J = 7.6 Hz, 1H), |
| | (second eluting | | | | 1.72 (d, J = 1.1 Hz, 3H), 0.90 |
| | isomer by SFC using | | | | (dq, J = 7.4, 2.4 Hz, 3H) ppm. |
| | a Whelk-O1 column) | | | | |

The following compounds were made using a method similar to that described in Example 2 except that 5-(methylthio)pyridin-3-amine was used as the coupling partner in step 2. Oxidation using General Method F followed by N-oxide reduction, using conditions outlined below, was carried out on the products of step 2 prior to SFC separation.

N-oxide reduction: To a solution of rac-3-((2R,3S,4S,5R)-3-(2-(difluoromethoxy)-3,4-difluorophenyl)-4,5-dimethyl-5-(trifluoromethyl)tetrahydrofuran-2-carboxamido)-5-(methylsulfonyl)pyridine 1-oxide (220 mg, 0.39 mmol) in MeCN (0.4 mL) stirring under nitrogen was added 4,4,5,5-tetramethyl-2-(4,4,5,5-tetramethyl-1,3,2-dioxaborolan-2-yl)-1,3,2-dioxaborolane (105 mg, 0.41 mmol). The mixture was heated at 70° C. for 4 hours before being concentrated in vacuo. Purification by flash chromatography (SiO₂) gave rac-(2R,3S,4S,5R)-3-(2-(difluoromethoxy)-3,4-difluorophenyl)-4,5-dimethyl-N-(5-(methylsulfonyl)pyridin-3-yl)-1-(trifluoromethyl)tetrahydrofuran-2-carboxamide (205 mg, 96%). ESI-MS m/z calc. 544.0903, found 545.6 (M+18.; 543.7 (M−1)⁻.

In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 95 | rel-(2S,3R,4R,5S)-3- | 544.44 | 545.1 | 3.31 | ¹H NMR (500 MHz, |
| | (2-(difluoromethoxy)- | | | | Methanol-d4) δ 9.09-9.04 (m, |
| | 3,4-difluorophenyl)- | | | | 1H), 8.84 (d, J = 2.2 Hz, 1H), |
| | 4,5-dimethyl-N-(5- | | | | 8.76-8.69 (m, 1H), 7.38 (ddd, |
| | (methylsulfonyl)pyridin- | | | | J = 9.3, 5.5, 2.3 Hz, 1H), 7.34- |
| | 3-y1)-5- | | | | 7.27 (m, 1H), 6.98 (td, J = |
| | (trifluoromethyl)tetra- | | | | 73.1, 1.2 Hz, 1H), 5.18 (d, J = |
| | hydrofuran-2- | | | | 10.6 Hz, 1H), 4.41 (dd, J = |
| | carboxamide | | | | 10.6, 8.0 Hz, 1H), 3.23 (s, 3H), |
| | (first eluting isomer by | | | | 2.88 (p, J = 7.6 Hz, 1H), 1.73 |
| | SFC using a Whelk- | | | | (d, J = 1.1 Hz, 3H), 0.91 (dq, |
| | O1 column) | | | | J = 7.4, 2.4 Hz, 3H) ppm. |
| 96 | rel-(2R,3S,4S,5R)-3- | 544.44 | 545.1 | 3.3 | ¹H NMR (500 MHz, |
| | (2-(difluoromethoxy)- | | | | Methanol-d4) δ 9.06 (d, J = 2.4 |
| | 3,4-difluorophenyl)- | | | | Hz, 1H), 8.85 (d, J = 2.0 Hz, |
| | 4,5-dimethyl-N-(5- | | | | 1H), 8.72 (t, J = 2.3 Hz, 1H), |
| | (methylsulfonyl)pyridin- | | | | 7.37 (ddd, J = 9.1, 5.5, 2.2 Hz, |
| | 3-yl)-5- | | | | 1H), 7.30 (td, J = 9.4, 7.5 Hz, |
| | (trifluoromethyl)tetra- | | | | 1H), 6.98 (td, J = 73.1, 1.2 Hz, |
| | hydrofuran-2- | | | | 1H), 5.18 (d, J = 10.6 Hz, 1H), |
| | carboxamide | | | | 4.41 (dd, J = 10.6, 8.0 Hz, 1H), |
| | (second eluting isomer | | | | 3.23 (s, 3H), 2.88 (p, J = 7.7 |
| | by SFC using a | | | | Hz, 1H), 1.73 (d, J = 1.1 Hz, |
| | Whelk-O1 column) | | | | 3H), 0.91 (dq, J = 7.6, 2.4 Hz, |
| | | | | | 3H) ppm. |

The following compound was made using a method similar to that described in Example 2, except that rac-(2R,3S,4S,5R)-3-(2-(difluoromethoxy)-4-fluoro-3-methylphenyl)-4,5-dimethyl-5-(trifluoromethyl)tetrahydrofuran-2-carboxylic acid was used as the starting material for step 2. Rac-(2R,3S,4S,5R)-3-(2-(difluoromethoxy)-4-fluoro-3-methylphenyl)-4,5-dimethyl-5-(trifluoromethyl)tetrahydrofuran-2-carboxylic acid was prepared using methods analogous to those described for other intermediates of this application. 2-methylsulfanylpyridin-4-amine (Hydrochloride salt) was used as the amine in step 2, SFC in step 3 was omitted and General Method D was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 97 | rac-(2R,3S,4S,5R)-3-2- | 540.476 | 541.1 | 1 | ¹H NMR (500 MHz, Chloroform- |
| | (difluoromethoxy)-4- | | | | d) δ 8.64 (s, 1H), 8.59 (d, J = 5.5 |
| | fluoro-3- | | | | Hz, 1H), 8.04-7.98 (m, 1H), 7.99- |
| | methylpheny1)-4,5- | | | | 7.93 (m, 1H), 7.32-7.27 (m, |
| | dimethyl-N-(2- | | | | 1H), 7.06 (t, J = 8.6 Hz, 1H), 6.33 |
| | (methylsulfonyl)pyridin- | | | | (dd, J = 73.9, 73.0 Hz, 1H), 4.97 |
| | 4-y1)-5- | | | | (d, J = 11.3 Hz, 1H), 4.19 (dd, J = |
| | (trifluoromethyl)tetra- | | | | 11.3, 8.1 Hz, 1H), 3.22 (s, 3H), |
| | hydrofuran-2- | | | | 2.79 (p, J = 7.6 Hz, 1H), 2.24 (d, |
| | carboxamide | | | | J = 2.1 Hz, 3H), 2.00 (s, 3H), 0.83 |
| | | | | | (dq, J = 7.5, 2.3 Hz, 3H) ppm. |

The following compound was made using a method similar to that described in Example 2, except that rac-(2R,3S,4S,5R)-3-(2-(difluoromethoxy)-4-fluoro-3-methylphenyl)-4,5-dimethyl-5-(trifluoromethyl)tetrahydrofuran-2-carboxylic acid was used as the starting material for step 2. Rac-(2R,3S,4S,5R)-3-(2-(difluoromethoxy)-4-fluoro-3-methylphenyl)-4,5-dimethyl-5-(trifluoromethyl)tetrahydrofuran-2-carboxylic acid was prepared using methods analogous to those described for other intermediates of this application. 4-amino-1-methyl-pyridin-2-one (Hydrochloride salt) was used as the amine in step 2 and purification by chiral SFC in step 3 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 98 | rac-(2R,3S,4S,5R)-3- | 492.411 | 493.1 | 3.22 | ¹H NMR (500 MHz, DMSO- |
| | (2-(difluoromethoxy)- | | | | d₆) δ 10.25 (s, 1H), 7.58 (d, J = |
| | 4-fluoro-3- | | | | 7.4 Hz, 1H), 7.32-6.88 (m, |
| | methylphenyl)-4,5- | | | | 3H), 6.71 (d, J = 2.3 Hz, 1H), |
| | dimethyl-N-(1-methyl- | | | | 6.38 (dd, J = 7.5, 2.4 Hz, 1H), |
| | 2-oxo-1,2- | | | | 5.02 (d, J = 10.5 Hz, 1H), 4.27 |
| | dihydropyridin-4-yl)- | | | | (dd, J = 10.4, 7.6 Hz, 1H), 3.32 |
| | 5- | | | | (s, 3H), 2.72 (t, J = 7.5 Hz, |
| | (trifluoromethyl)tetra- | | | | 1H), 2.16 (d, J = 2.0 Hz, 3H), |
| | hydrofuran-2- | | | | 1.55 (s, 3H), 0.72 (d, J = 7.3 |
| | carboxamide | | | | Hz, 3H) ppm. |

The following compounds were made using a method similar to that described in Example 2, except that rac-(2R,3S,4S,5R)-3-(2-(difluoromethoxy)-4-fluoro-3-methylphenyl)-4,5-dimethyl-5-(trifluoromethyl)tetrahydrofuran-2-carboxylic acid was used as the starting material for step 2. Rac-(2R,3S,4S,5R)-3-(2-(difluoromethoxy)-4-fluoro-3-methylphenyl)-4,5-dimethyl-5-(trifluoromethyl)tetrahydrofuran-2-carboxylic acid was prepared using methods analogous to those described for other intermediates of this application. 4-amino-1-methyl-pyridin-2-one (Hydrochloride salt) was used as the amine in step 2 and purification by chiral SFC in step 3 was carried out using a (R,R)-Whelk-O1 column, 5 μm particle size, 25 cm×21.2 mm from Regis Technologies. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 99 | rel-(2S,3R,4R,5S)-3- | 492.411 | 493.1 | 3.23 | ¹H NMR (500 MHz, DMSO- |
| | (2-(difluoromethoxy)- | | | | d6) δ 10.26 (s, 1H), 7.60 (d, |
| | 4-fluoro-3- | | | | J = 7.4 Hz, 1H), 7.30 (dd, J = |
| | methylphenyl)-4,5- | | | | 8.9, 6.3 Hz, 1H), 7.26-6.88 |
| | dimethyl-N-(1-methyl- | | | | (m, 2H), 6.72 (d, J = 2.3 Hz, |
| | 2-oxo-1,2- | | | | 1H), 6.40 (dd, J = 7.4, 2.4 Hz, |
| | dihydropyridin-4-yl)- | | | | 1H), 5.04 (d, J = 10.4 Hz, 1H), |
| | 5- | | | | 4.28 (dd, J = 10.5, 7.5 Hz, 1H), |
| | (trifluoromethyl)tetra- | | | | 3.34 (s, 3H), 2.73 (p, J = 7.5 |
| | hydrofuran-2- | | | | Hz, 1H), 2.18 (d, J = 2.0 Hz, |
| | carboxamide | | | | 3H), 1.57 (s, 3H), 0.77-0.68 |
| | | | | | (m, 3H) ppm. |
| 100 | rel-(2R,3S,4S,5R)-3- | 492.411 | 493.1 | 3.22 | ¹H NMR (500 MHz, DMSO- |
| | (2-(difluoromethoxy)- | | | | d6) δ 10.26 (s, 1H), 7.60 (d, |
| | 4-fluoro-3- | | | | J = 7.4 Hz, 1H), 7.30 (dd, J = |
| | methylphenyl)-4,5- | | | | 8.7, 6.3 Hz, 1H), 7.27-6.88 |
| | dimethyl-N-(1-methyl- | | | | (m, 2H), 6.72 (d, J = 2.3 Hz, |
| | 2-oxo-1,2- | | | | 1H), 6.40 (dd, J = 7.4, 2.4 Hz, |
| | dihydropyridin-4-yl)- | | | | 1H), 5.04 (d, J = 10.4 Hz, 1H), |
| | 5- | | | | 4.28 (dd, J = 10.4, 7.4 Hz, 1H), |
| | (trifluoromethyl)tetra- | | | | 3.33 (s, 3H), 2.74 (q, J = 7.5 |
| | hydrofuran-2- | | | | Hz, 1H), 2.18 (d, J = 2.1 Hz, |
| | carboxamide | | | | 3H), 1.56 (s, 3H), 0.73 (d, J = |
| | (second eluting isomer | | | | 7.3 Hz, 3H) ppm. |
| | by SFC) | | | | |

### Example 3

rel-(2S,3R,4R,5S)-3-(2-ethoxy-3,4-difluorophenyl)-4,5-dimethyl-N-(1-methyl-2-oxo-1,2-dihydropyridin-4-yl)-5-(trifluoromethyl)tetrahydrofuran-2-carboxamide (102) and rel-(2R,3S,4S,5R)-3-(2-ethoxy-3,4-difluorophenyl)-4,5-dimethyl-N-(1-methyl-2-oxo-1,2-dihydropyridin-4-yl)-5-(trifluoromethyl)tetrahydrofuran-2-carboxamide (103)

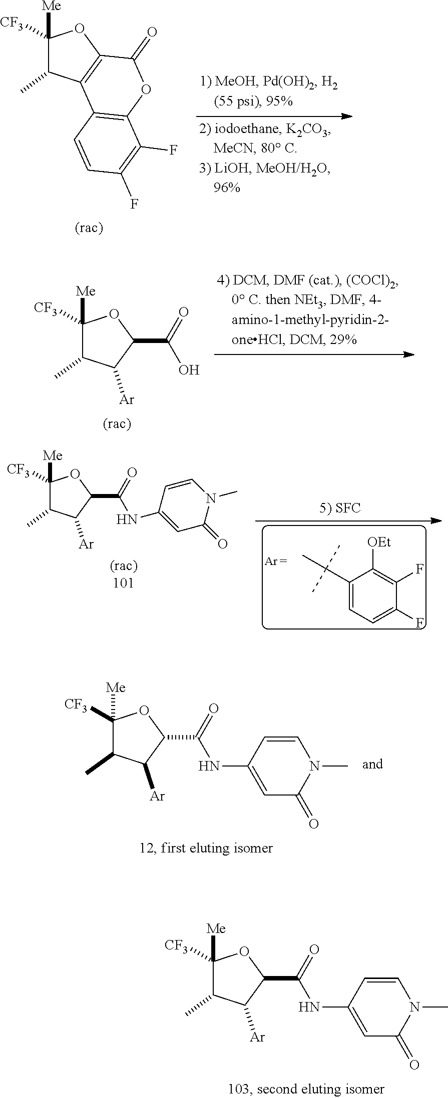

### Step 1:

MeOH (620 mL) was added into a Parr shaker flask shaker containing rac-(1S,2R)-6,7-difluoro-1,2-dimethyl-2-(trifluoromethyl)-1,2-dihydro-4H-furo[2,3-c]chromen-4-one (32.3 g, 100.9 mmol) and Pd(OH)₂ (24 g, 34.18 mmol). The mixture was degassed and repressurised to 55 psi hydrogen, and left to shake for 2 days. The mixture was filtered, washing the catalyst with DCM followed by EtOAc and methanol, and the filtrate concentrated in vacuo to give methyl rac-(2S,3S,4S,5R)-3-(3,4-difluoro-2-hydroxy-phenyl)-4,5-dimethyl-5-(trifluoromethyl)tetrahydrofuran-2-carboxylate (34 g, 95%). ¹H NMR (500 MHz, Methanol-d₄) δ 7.05 (ddt, J=9.1, 7.5, 2.0 Hz, 1H), 6.57 (ddd, J=10.1, 9.0, 7.6 Hz, 1H), 5.01 (d, J=6.0 Hz, 1H), 4.34 (dd, J=8.5, 6.0 Hz, 1H), 3.49 (s, 3H), 2.93 (h, J=7.4 Hz, 1H), 1.50 (d, J=1.2 Hz, 3H), 0.89 (dd, J=7.6, 1.9 Hz, 3H) ppm. ESI-MS m/z calc. 354.08905, found 353.6 (M−1)⁻.

### Step 2:

To a solution of rac-(2S,3S,4S,5R)-3-(3,4-difluoro-2-hydroxy-phenyl)-4,5-dimethyl-5-(trifluoromethyl)tetrahydrofuran-2-carboxylate (7 g, 20.57 mmol) in acetonitrile (42 mL) was added K₂CO₃ (11.4 g, 82.49 mmol) and iodoethane (7.2 mL, 90.02 mmol) and the reaction heated to 80° C. for 4.5 hours. The reaction was cooled to ambient temperature and diluted with EtOAc (70 mL), filtered (washing the pad with a further 70 mL EtOAc) and then the filtrate concentrated in vacuo to give rac-ethyl (3S,4S,5R)-3-(2-ethoxy-3,4-difluorophenyl)-4,5-dimethyl-5-(trifluoromethyl)tetrahydrofuran-2-carboxylate (6.39 g, 78%) as an orange oil. ¹H NMR (500 MHz, Chloroform-d) δ 6.97-6.93 (m, 1H), 6.89-6.84 (m, 1H), 4.90 (d, J=10.4 Hz, 1H), 4.34-4.24 (m, 1H), 4.20-4.11 (m, 4H), 2.74 (p, J=7.6 Hz, 1H), 1.65 (d, J=1.2 Hz, 3H), 1.43 (td, J=7.0, 0.7 Hz, 3H), 1.21 (t, J=7.1 Hz, 3H), 0.79 (dq, J=7.4, 2.4 Hz, 3H) ppm. ¹⁹F NMR (471 MHz, Chloroform-d) δ−74.61, −137.35 (d, J=19.8 Hz), −153.97 (d, J=19.9 Hz) ppm; ESI-MS m/z calc. 396.136, found 397.7 (M+1)⁺; Retention time: 1.1 minutes.

### Step 3:

LiOH (17 mL of 2 M, 34.00 mmol) was added to a stirred solution of rac-ethyl (3S,4S,5R)-3-(2-ethoxy-3,4-difluorophenyl)-4,5-dimethyl-5-(trifluoromethyl)tetrahydrofuran-2-carboxylate (6.3874 g, 16.12 mmol) in methanol (70 mL)/water (20 mL) and the mixture stirred at ambient temperature for 2 hours. The reaction was concentrated in vacuo and partitioned between MTBE (30 mL) and 1M HCl (20 mL). The layers were separated and the aqueous layer extracted with MTBE (2×20 mL). The combined organic extracts were dried (MgSO₄), filtered and concentrated in vacuo to give rac-(2R,3S,4S,5R)-3-(2-ethoxy-3,4-difluorophenyl)-4,5-dimethyl-5-(trifluoromethyl)tetrahydrofuran-2-carboxylic acid (6.4494 g, 96%) as an orange oil that solidifies on standing. ¹H NMR (500 MHz, Chloroform-d) δ 6.95 (ddd, J=7.9, 5.5, 2.0 Hz, 1H), 6.85 (td, J=9.2, 7.3 Hz, 1H), 4.92 (d, J=10.8 Hz, 1H), 4.28 (dqd, J=8.9, 7.0, 1.8 Hz, 1H), 4.21-4.08 (m, 2H), 2.73 (p, J=7.6 Hz, 1H), 1.61 (d, J=1.3 Hz, 3H), 1.39 (td, J=7.1, 0.7 Hz, 3H), 0.76 (dq, J=7.3, 2.3 Hz, 3H) ppm. ESI-MS m/z calc. 368.1047, found 367.5 (M+1)⁺; Retention time: 0.59 minutes.

### Step 4:

A solution of rac-(2R,3S,4S,5R)-3-(2-ethoxy-3,4-difluorophenyl)-4,5-dimethyl-5-(trifluoromethyl)tetrahydrofuran-2-carboxylic acid (150 mg, 0.3617 mmol) in DCM (3 mL) was cooled using an ice-bath. To this was added DMF (6 μL, 0.07749 mmol) (1 drop DMF) followed by careful addition of oxalyl chloride (100 μL, 1.146 mmol). Solution was stirred with ice-bath in place for 20 mins before being concentrated in-vacuo and azeotroped with DCM to afford a cream solid. This acid chloride was taken up in DCM (3 mL) and added to an ice bath cooled solution of 4-amino-1-methyl-pyridin-2-one (Hydrochloride salt) (64 mg, 0.3985 mmol) and DIPEA (243 μL, 1.395 mmol) in DCM (3 mL). The resulting suspension was stirred with ice-bath in place for 1 hr and then at RT for the weekend. The reaction mixture was then partitioned with DCM and water, layers separated using a phase separation cartridge and the organics were concentrated in-vacuo. The residue was purified by flash chromatography (4 g SiO₂, 0 to 50% EtOAc in heptane, loaded on Telos nm) to afford rac-(2R,3S,4S,5R)-3-(2-ethoxy-3,4-difluorophenyl)-4,5-dimethyl-N-(1-methyl-2-oxo-1,2-dihydropyridin-4-yl)-5-(trifluoromethyl)tetrahydrofuran-2-carboxamide (101, 53.9 mg, 29%) as a yellow oil. ¹H NMR (500 MHz, DMSO-d₆) δ 10.28 (s, 1H), 7.59 (d, J=7.5 Hz, 1H), 7.16 (td, J=9.5, 7.5 Hz, 1H), 7.12-7.03 (m, 1H), 6.73 (d, J=2.4 Hz, 1H), 6.38 (dd, J=7.4, 2.4 Hz, 1H), 5.03 (d, J=10.4 Hz, 1H), 4.29-4.10 (m, 3H), 3.33 (s, 3H), 2.73 (p, J=7.4 Hz, 1H), 1.57 (s, 3H), 1.35 (t, J=7.0 Hz, 3H), 0.75-0.66 (m, 3H) ppm. ¹⁹F NMR (471 MHz, DMSO-d₆) δ−73.42, −138.10 (d, J=2 1.4 Hz), −152.65-−156.46 (m) ppm. ESI-MS m/z calc. 474.1578, found 475.1 (M+1); 473.0 (M−1)⁻; Retention time: 3.21 minutes.

### Step 5:

rac-(2R,3S,4S,5R)-3-(2-ethoxy-3,4-difluorophenyl)-4,5-dimethyl-N-(1-methyl-2-oxo-1,2-dihydropyridin-4-yl)-5-(trifluoromethyl)tetrahydrofuran-2-carboxamide (51.2 mg, 0.09713 mmol) was purified by chiral SFC using a (R,R)-Whelk-01 column, 5 μm particle size, 25 cm×21.2 mm from Regis Technologies to give:

First eluting isomer (rt=2.48 minutes): rel-(2S,3R,4R,5S)-3-(2-ethoxy-3,4-difluorophenyl)-4,5-dimethyl-N-(1-methyl-2-oxo-1,2-dihydropyridin-4-yl)-5-(trifluoromethyl)tetrahydrofuran-2-carboxamide (102, 14.5 mg, 59%). ¹H NMR (500 MHz, DMSO-d₆) δ 10.28 (s, 1H), 7.60 (d, J=7.4 Hz, 1H), 7.17 (dt, J=9.8, 8.2 Hz, 1H), 7.12-7.05 (m, 1H), 6.74 (d, J=2.4 Hz, 1H), 6.39 (dd, J=7.4, 2.4 Hz, 1H), 5.04 (d, J=10.4 Hz, 1H), 4.30-4.11 (m, 3H), 3.34 (s, 3H), 2.74 (p, J=7.5 Hz, 1H), 1.58 (s, 3H), 1.36 (t, J=7.0 Hz, 3H), 0.78-0.64 (m, 3H) ppm. ¹⁹F NMR (471 MHz, DMSO-d₆) δ−73.42, −138.11 (d, J=21.6 Hz), −154.41 (d, J=21.6 Hz) ppm. ESI-MS m/z calc. 474.1578, found 475.6 (M+1)⁺; 473.5 (M−1)⁻; Retention time: 3.24 minutes.

Second eluting isomer (rt=4.07 minutes): rel-(2R,3S,4S,5R)-3-(2-ethoxy-3,4-difluorophenyl)-4,5-dimethyl-N-(1-methyl-2-oxo-1,2-dihydropyridin-4-yl)-5-(trifluoromethyl)tetrahydrofuran-2-carboxamide (103, 13.64 mg, 58%). ¹H NMR (500 MHz, DMSO-d₆) δ 10.30 (s, 1H), 7.58 (d, =7.4 Hz, 1H), 7.22-7.02 (m, 2H), 6.73 (d, 1=2.3 Hz, 1H), 6.38 (dd, J=7.5, 2.4 Hz, 1H), 5.03 (d, (=10.4 Hz, 1H), 4.29-4.07 (m, 3H), 3.32 (s, 3H), 2.73 (q, J=7.6 Hz, 1H), 1.57 (s, 3H), 1.34 (t, i=7.0 Hz, 3H), 0.76-0.65 (n, 3H) ppm. ¹⁹F NMR (471 MHz, DMSO-d₆) δ−73.42, −138.11 (d, J=2 1.3 Hz), −15 4.41 (d, =2 1.3 Hz) ppm. ESI-MS m/z calc. 474.1578, found 475.6 (M+1)⁺; 473.7 (M−1)⁻; Retention time: 3.23 minutes.

The following compounds were made using the method described in Example 3, except that (2R,3S,4S,5R)-3-(2-ethoxy-3,4-difluorophenyl)-4,5-dimethyl-5-(trifluoromethyl)tetrahydrofuran-2-carboxylic acid and (R) or (S)-2-(2,2-dimethyl-1,3-dioxolan-4-yl)pyridin-4-amine (first or second eluting isomer by SFC) were used in the amide coupling step 4 and General Method B was used as the final step. (2R,3S,4S,5R)-3-(2-ethoxy-3,4-difluorophenyl)-4,5-dimethyl-5-(trifluoromethyl)tetrahydrofuran-2-carboxylic acid was made using similar methods to those described in Example 7. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 104 | rel- | 504.447 | 505.4 | 3.13 | ¹H NMR (500 MHz, DMSO-d₆) |
| | (2R*,3S*,4S*,5R*)- | | | | δ 10.56 (s, 1H), 8.34 (d, J = 5.6 |
| | N-(2-(1,2- | | | | Hz, 1H), 7.71 (d, J = 2.3 Hz, |
| | dihydroxyethyl)pyridin- | | | | 1H), 7.50 (dd, J = 5.5, 2.2 Hz, |
| | 4-yl)-3-(2-ethoxy- | | | | 1H), 7.15 (pd, J = 9.0, 6.3 Hz, |
| | 3,4-difluorophenyl)- | | | | 2H), 5.38 (d, J = 4.7 Hz, 1H), |
| | 4,5-dimethyl-5- | | | | 5.08 (d, J = 10.5 Hz, 1H), 4.65 |
| | (trifluoromethyl)tetra- | | | | (t, J = 5.9 Hz, 1H), 4.52 (dt, J = |
| | hydrofuran-2- | | | | 6.8, 4.3 Hz, 1H), 4.28 (dd, J = |
| | carboxamide | | | | 10.6, 7.5 Hz, 1H), 4.24-4.10 |
| | (first eluting isomer | | | | (m, 2H), 3.65 (ddd, J = 11.0, |
| | by SFC of 2-(2,2- | | | | 6.0, 4.1 Hz, 1H), 3.43 (dt, J = |
| | dimethyl-1,3- | | | | 10.9, 6.3 Hz, 1H), 2.75 (p, J = |
| | dioxolan-4- | | | | 7.5 Hz, 1H), 1.60 (s, 3H), 1.35 |
| | yl)pyridin-4-amine | | | | (t, J = 7.0 Hz, 3H), 0.76-0.69 |
| | used in step 4) | | | | (m, 3H) ppm. |
| 105 | rel- | 504.447 | 505.4 | 3.13 | ¹H NMR (500 MHz, DMSO-d₆) |
| | (2R*,3S*,4S*,5R*)- | | | | δ 10.57 (s, 1H), 8.34 (d, J = 5.3 |
| | N-(2-(1,2- | | | | Hz, 1H), 7.71 (s, 1H), 7.51 (dd, |
| | dihydroxyethyl)pyridin- | | | | J = 5.9, 2.0 Hz, 1H), 7.22-7.06 |
| | 4-yl)-3-(2-ethoxy- | | | | (m, 2H), 5.38 (d, J = 4.6 Hz, |
| | 3,4-difluorophenyl)- | | | | 1H), 5.08 (d, J = 10.5 Hz, 1H), |
| | 4,5-dimethyl-5- | | | | 4.65 (t, J = 5.9 Hz, 1H), 4.58- |
| | (trifluoromethyl)tetra- | | | | 4.48 (m, 1H), 4.28 (dd, J = 10.5, |
| | hydrofuran-2- | | | | 7.5 Hz, 1H), 4.25-4.07 (m, |
| | carboxamide | | | | 2H), 3.65 (ddd, J = 10.2, 5.8, 3.9 |
| | (second eluting | | | | Hz, 1H), 3.44 (dt, J = 11.6, 6.2 |
| | isomer by SFC of 2- | | | | Hz, 1H), 2.75 (p, J = 7.5 Hz, |
| | (2,2-dimethyl-1,3- | | | | 1H), 1.60 (s, 3H), 1.35 (t, J = 7.0 |
| | dioxolan-4- | | | | Hz, 3H), 0.72 (dd, J = 7.6, 2.4 |
| | yl)pyridin-4-amine | | | | Hz, 3H) ppm. |
| | used in step 4) | | | | |

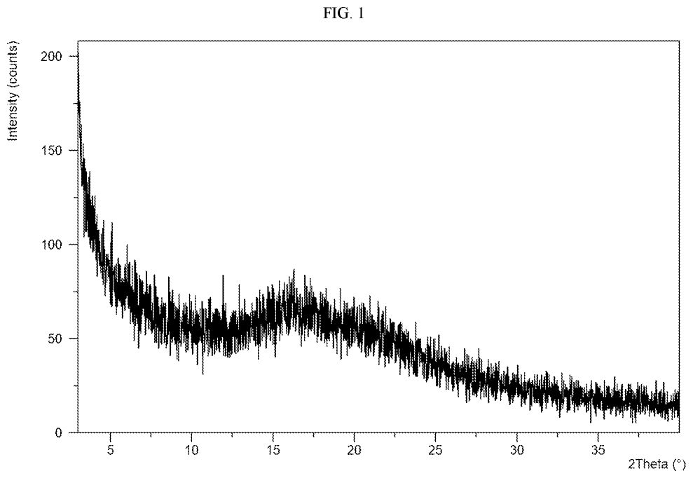

Compound 105 was analyzed by X-ray powder diffraction and determined to be amorphous (see FIG. 1).

The following compounds were made using the method described in Example 3, except that 5-(methylthio)pyridin-3-amine was used in the amide coupling step 4 and General Method D was used prior(to chiral SFC purification using a (R,R)-Whelk-O41 column, 5 μm particle size, 25 cm×21.2 mm from Regis Technologies. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 106 | rel-(2S,3R,4R,5S)- | 522.485 | 523.1 | 3.39 | ¹H NMR (500 MHz, Chloroform- |
| | 3-(2-ethoxy-3,4- | | | | d) δ 8.93 (d, J = 2.5 Hz, 1H), 8.90 |
| | difluorophenyl)- | | | | (d, J = 2.0 Hz, 1H), 8.63 (t, J = 2.3 |
| | 4,5-dimethyl-N-(5- | | | | Hz, 1H), 8.57 (s, 1H), 7.09-7.05 |
| | (methylsulfonyl)py- | | | | (m, 1H), 6.91 (td, J = 9.2, 7.4 Hz, |
| | ridin-3-yl)-5- | | | | 1H), 5.05 (d, J = 11.2 Hz, 1H), |
| | (trifluoromethyl)tet- | | | | 4.34-4.28 (m, 1H), 4.25-4.19 |
| | rahydrofuran-2- | | | | (m, 1H), 4.13 (dd, J = 11.2, 7.8 |
| | carboxamide | | | | Hz, 1H), 3.10 (s, 3H), 2.79 (p, J = |
| | (First eluting peak | | | | 7.6 Hz, 1H), 1.70 (s, 3H), 1.39 (t, |
| | by SFC) | | | | J = 7.0 Hz, 3H), 0.80 (dq, J = 7.4, |
| | | | | | 2.1 Hz, 3H) ppm. |
| 107 | rel-(2R,3S,4S,5R)- | 522.485 | 523.1 | 3.39 | ¹H NMR (500 MHz, Chloroform- |
| | 3-(2-ethoxy-3,4- | | | | d) δ 8.96 (d, J = 2.5 Hz, 1H), 8.93 |
| | difluorophenyl)- | | | | (d, J = 2.1 Hz, 1H), 8.65 (t, J = 2.2 |
| | 4,5-dimethyl-N-(5- | | | | Hz, 1H), 8.60 (s, 1H), 7.11-7.07 |
| | (methylsulfonyl)py- | | | | (m, 1H), 6.93 (td, J = 9.2, 7.4 Hz, |
| | ridin-3-yl)-5- | | | | 1H), 5.07 (d, J = 11.3 Hz, 1H), |
| | (trifluoromethyl)tet- | | | | 4.37-4.30 (m, 1H), 4.28-4.21 |
| | rahydrofuran-2- | | | | (m, 1H), 4.15 (dd, J = 11.2, 7.8 |
| | carboxamide | | | | Hz, 1H), 3.12 (s, 3H), 2.82 (p, J = |
| | (Second eluting | | | | 7.6 Hz, 1H), 1.72 (s, 3H), 1.42 (t, |
| | peak by SFC) | | | | J = 7.0 Hz, 3H), 0.82 (dt, J = 7.4, |
| | | | | | 2.3 Hz, 3H) ppm. |

The following compounds were made using the method described in Example 3, except that 4-methylsulfonylpyridin-2-amine was used in the amide coupling step 4. The purification in step 5 was conducted by chiral SFC using a Lux Cellulose-2 column, 5)m particle size, 25 cm×10 mm from Phenomenex, Inc. on a Minigram SFC instrument from Berger Instruments and General Method D was used as the final step on separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 4 | rel-(2S,3R,4R,5S)- | 522.485 | 523.5 | 3.52 | ¹H NMR (500 MHz, DMSO-d6) δ |
| | 3-(2-ethoxy-3,4- | | | | 10.92 (s, 1H), 8.65 (d, J = 5.5 Hz, |
| | difluorophenyl)- | | | | 1H), 8.38 (d, J = 2.0 Hz, 1H), 7.91 |
| | 4,5-dimethyl-N-(2- | | | | (dd, J = 5.5, 2.1 Hz, 1H), 7.22- |
| | (methylsulfony1)py- | | | | 7.14 (m, 2H), 5.14 (d, J = 10.4 |
| | ridin-4-yl)-5- | | | | Hz, 1H), 4.29 (dd, J = 10.4, 7.5 |
| | (trifluoromethyl)tet- | | | | Hz, 1H), 4.25-4.12 (m, 2H), 3.25 |
| | rahydrofuran-2- | | | | (s, 3H), 2.77 (p, J = 7.5 Hz, 1H), |
| | carboxamide | | | | 1.62 (s, 3H), 1.35 (t, J = 7.0 Hz, |
| | (precursor was first | | | | 3H), 0.78-0.70 (m, 3H) ppm. |
| | eluting peak by | | | | |
| | SFC) | | | | |
| 108 | rel-(2R,3S,4S,5R)- | 522.485 | 523.6 | 3.53 | ¹H NMR (500 MHz, DMSO-d6) δ |
| | 3-(2-ethoxy-3,4- | | | | 10.93 (s, 1H), 8.65 (d, J = 5.4 Hz, |
| | difluorophenyl)- | | | | 1H), 8.38 (d, J = 2.0 Hz, 1H), 7.91 |
| | 4,5-dimethyl-N-(2- | | | | (dd, J = 5.5, 2.1 Hz, 1H), 7.23- |
| | (methylsulfonyl)py- | | | | 7.10 (m, 2H), 5.14 (d, J = 10.3 |
| | ridin-4-yl)-5- | | | | Hz, 1H), 4.30 (dd, J = 10.4, 7.5 |
| | (trifluoromethyl)tet- | | | | Hz, 1H), 4.27-4.11 (m, 2H), 3.25 |
| | rahydrofuran-2- | | | | (s, 3H), 2.77 (p, J = 7.5 Hz, 1H), |
| | carboxamide | | | | 1.62 (s, 3H), 1.35 (t, J = 7.0 Hz, |
| | (precursor was | | | | 3H), 0.79-0.70 (m, 3H) ppm. |
| | second eluting peak | | | | |
| | by SFC) | | | | |

The following compounds were made using the method described in Example 3, except that 4-methylsulfonylpyridin-2-amine was used in the amide coupling step 4. The purification in step 5 was conducted by chiral SFC using a Lux Cellulose-2 column, 5 μm particle size, 25 cm×10 mm from Phenomenex, Inc. on a Minigram SFC instrument from Berger Instruments and step 1 from General Method G was used on the separated isomers as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 109 | rel-(2S,3R,4R,5S)-3- | 521.501 | 522.6 | 3.26 | ¹H NMR (500 MHz, DMSO-d₆) |
| | (2-ethoxy-3,4- | | | | δ 10.89 (s, 1H), 8.58 (d, J = 5.5 |
| | difluorophenyl)-4,5- | | | | Hz, 1H), 8.36 (dd, J = 4.3, 2.0 |
| | dimethyl-N-(2-(S- | | | | Hz, 1H), 7.82 (ddd, J = 5.7, 3.8, |
| | methylsulfonimidoyl)py- | | | | 2.1 Hz, 1H), 7.22-7.14 (m, |
| | ridin-4-yl)-5- | | | | 2H), 5.12 (d, J = 10.4 Hz, 1H), |
| | (trifluoromethyl)tetra- | | | | 4.37-4.27 (m, 2H), 4.26-4.12 |
| | hydrofuran-2- | | | | (m, 2H), 3.12 (d, J = 1.0 Hz, |
| | carboxamide | | | | 3H), 2.77 (p, J = 7.4 Hz, 1H), |
| | as a mixture of | | | | 1.62 (s, 3H), 1.36 (t, J = 7.0 Hz, |
| | epimers at the | | | | 3H), 0.79-0.69 (m, 3H) ppm. |
| | sulfonimidoyl | | | | |
| | position | | | | |
| | (precursor was first | | | | |
| | eluting peak by SFC | | | | |
| | on Lux-cellulose-2 | | | | |
| | column) | | | | |
| 110 | rel-(2R,3S,4S,5R)-3- | 521.501 | 522.6 | 3.26 | ¹H NMR (500 MHz, DMSO-d₆) |
| | (2-ethoxy-3,4- | | | | δ 10.89 (s, 1H), 8.58 (d, J = 5.5 |
| | difluorophenyl)-4,5- | | | | Hz, 1H), 8.36 (dd, J = 4.4, 2.0 |
| | dimethyl-N-(2-(S- | | | | Hz, 1H), 7.82 (ddd, J = 5.7, 3.7, |
| | methylsulfonimidoyl)py- | | | | 2.1 Hz, 1H), 7.21-7.12 (m, |
| | ridin-4-yl)-5- | | | | 2H), 5.12 (d, J = 10.4 Hz, 1H), |
| | (trifluoromethyl)tetra- | | | | 4.37-4.26 (m, 2H), 4.26-4.10 |
| | hydrofuran-2- | | | | (m, 2H), 3.12 (d, J = 1.0 Hz, |
| | carboxamide | | | | 3H), 2.76 (p, J = 7.5 Hz, 1H), |
| | as a mixture of | | | | 1.62 (s, 3H), 1.36 (t, J = 7.0 Hz, |
| | epimers at the | | | | 3H), 0.80-0.69 (m, 3H) ppm. |
| | sulfonimidoyl | | | | |
| | position | | | | |
| | (precursor was | | | | |
| | second eluting peak | | | | |
| | by SFC on Lux- | | | | |
| | cellulose-2 column) | | | | |

The following compounds were made using the method described in Example 3, except that 4-methylsulfonylpyridin-2-amine was used in the amide coupling step 4. The purification in step 5 was conducted by chiral SFC using a Lux Cellulose-2 column, 5 μm particle size, 25 cm×10 mm from Phenomenex, Inc. on a Minigram SFC instrument from Berger Instruments and General Method G was used on the separated isomers as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 10 | rel-(2S,3R,4R,5S)-3- | 521.501 | 522.6 | 3.26 | ¹H NMR (500 MHz, DMSO-d₆) |
| | (2-ethoxy-3,4- | | | | δ 10.89 (s, 1H), 8.57 (d, J = 5.5 |
| | difluorophenyl)-4,5- | | | | Hz, 1H), 8.36 (d, J = 2.0 Hz, |
| | dimethyl-N-(2-(S- | | | | 1H), 7.81 (dd, J = 5.5, 2.0 Hz, |
| | methylsulfonimidoyl)py- | | | | 1H), 7.22-7.11 (m, 2H), 5.12 |
| | ridin-4-yl)-5- | | | | (d, J = 10.4 Hz, 1H), 4.36-4.24 |
| | (trifluoromethyl)tetra- | | | | (m, 2H), 4.24-4.10 (m, 2H), |
| | hydrofuran-2- | | | | 3.12 (d, J = 1.0 Hz, 3H), 2.76 (p, |
| | carboxamide | | | | J = 7.5 Hz, 1H), 1.62 (s, 3H), |
| | (First eluting peak by | | | | 1.36 (t, J = 7.0 Hz, 3H), 0.78- |
| | SFC on (R,R) | | | | 0.67 (m, 3H) ppm. |
| | Whelk-O1 column; | | | | |
| | precursor came from | | | | |
| | first eluting peak by | | | | |
| | SFC on Lux- | | | | |
| | cellulose-2 column) | | | | |
| 11 | rel-(2S,3R,4R,5S)-3- | 521.501 | 522.5 | 3.26 | ¹H NMR (500 MHz, DMSO-d₆) |
| | (2-ethoxy-3,4- | | | | δ 10.90 (s, 1H), 8.57 (d, J = 5.5 |
| | difluorophenyl)-4,5- | | | | Hz, 1H), 8.35 (d, J = 2.0 Hz, |
| | dimethyl-N-(2-(S- | | | | 1H), 7.82 (dd, J = 5.5, 2.1 Hz, |
| | methylsulfonimidoyl)py- | | | | 1H), 7.23-7.07 (m, 2H), 5.12 |
| | ridin-4-yl)-5- | | | | (d, J = 10.4 Hz, 1H), 4.38-4.27 |
| | (trifluoromethyl)tetra- | | | | (m, 2H), 4.27-4.07 (m, 2H), |
| | hydrofuran-2- | | | | 3.12 (d, J = 1.1 Hz, 3H), 2.76 (p, |
| | carboxamide | | | | J = 7.5 Hz, 1H), 1.62 (s, 3H), |
| | (Second eluting peak | | | | 1.36 (t, J = 7.0 Hz, 3H), 0.79- |
| | by SFC on (R,R) | | | | 0.65 (m, 3H) ppm. |
| | Whelk-O1 column; | | | | |
| | precursor came from | | | | |
| | first eluting peak by | | | | |
| | SFC on Lux- | | | | |
| | cellulose-2 column) | | | | |
| 111 | rel-(2R,3S,4S,5R)-3- | 521.501 | 522.5 | 3.26 | ¹H NMR (500 MHz, DMSO-d₆) |
| | (2-ethoxy-3,4- | | | | δ 10.89 (s, 1H), 8.57 (d, J = 5.5 |
| | difluorophenyl)-4,5- | | | | Hz, 1H), 8.36 (d, J = 2.0 Hz, |
| | dimethyl-N-(2-(S- | | | | 1H), 7.81 (dd, J = 5.4, 2.1 Hz, |
| | methylsulfonimidoyl)py- | | | | 1H), 7.23-7.09 (m, 2H), 5.12 |
| | ridin-4-yl)-5- | | | | (d, J = 10.4 Hz, 1H), 4.36-4.27 |
| | (trifluoromethyl)tetra- | | | | (m, 2H), 4.27-4.09 (m, 2H), |
| | hydrofuran-2- | | | | 3.11 (d, J = 1.0 Hz, 3H), 2.77 (g, |
| | carboxamide | | | | J = 7.5 Hz, 1H), 1.61 (s, 3H), |
| | (First eluting peak by | | | | 1.35 (t, J = 7.0 Hz, 3H), 0.78- |
| | SFC on AD-H | | | | 0.69 (m, 3H) ppm. |
| | column; precursor | | | | |
| | came from second | | | | |
| | eluting peak by SFC | | | | |
| | on Lux-cellulose-2 | | | | |
| | column) | | | | |
| 112 | rel-(2R,3S,4S,5R)-3- | 521.501 | 522.5 | 3.25 | ¹H NMR (500 MHz, DMSO-d₆) |
| | (2-ethoxy-3,4- | | | | δ 10.89 (s, 1H), 8.57 (d, J = 5.5 |
| | difluorophenyl)-4,5- | | | | Hz, 1H), 8.35 (d, J = 1.9 Hz, |
| | dimethyl-N-(2-(S- | | | | 1H), 7.82 (dd, J = 5.5, 2.1 Hz, |
| | methylsulfonimidoyl)py- | | | | 1H), 7.22-7.09 (m, 2H), 5.12 |
| | ridin-4-yl)-5- | | | | (d, J = 10.4 Hz, 1H), 4.37-4.25 |
| | (trifluoromethyl)tetra- | | | | (m, 2H), 4.25-4.11 (m, 2H), |
| | hydrofuran-2- | | | | 3.12 (d, J = 1.0 Hz, 3H), 2.76 (p, |
| | carboxamide | | | | J = 7.4 Hz, 1H), 1.62 (s, 3H), |
| | (Second eluting peak | | | | 1.36 (t, J = 7.0 Hz, 3H), 0.77- |
| | by SFC on AD-H | | | | 0.66 (m, 3H) ppm. |
| | column; precursor | | | | |
| | came from second | | | | |
| | eluting peak by SFC | | | | |
| | on Lux-cellulose-2 | | | | |
| | column) | | | | |

The following compounds were made using the method described in Example 3, except that 2-(methylthio)pyridin-4-amine was used in the amide coupling step 4. The purification in step was conducted by chiral SFC using a Lux Cellulose-2 column, 7 0m particle size, 25 cm×10 mm from Phenomenex, Inc. on a Minigram SFC instrument from Berger Instruments and separated isomers were subjected to General Method G followed by General Method H as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 12 | rel-(2R,3S,4S,5R)-N- | 535.527 | 537.5 | 3.37 | ¹H NMR (500 MHz, DMSO-d6) |
| | (2-(N,S- | | | | δ 10.87 (s, 1H), 8.60 (d, J = 5.5 |
| | dimethylsulfonimido- | | | | Hz, 1H), 8.31 (d, J = 2.1 Hz, |
| | yl)pyridin-4-yl)-3-(2- | | | | 1H), 7.83 (dd, J = 5.5, 2.1 Hz, |
| | ethoxy-3,4- | | | | 1H), 7.23-7.08 (m, 2H), 5.10 |
| | difluorophenyl)-4,5- | | | | (d, J = 10.3 Hz, 1H), 4.28 (dd, J = |
| | dimethyl-5- | | | | 10.5, 7.6 Hz, 1H), 4.16 (ddd, J = |
| | (trifluoromethyl)tetra- | | | | 16.4, 8.1, 6.7 Hz, 2H), 3.14 (s, |
| | hydrofuran-2- | | | | 3H), 2.75 (p, J = 7.5 Hz, 1H), |
| | carboxamide | | | | 2.44 (s, 3H), 1.60 (s, 3H), 1.34 |
| | (first eluting isomer | | | | (t, J = 7.0 Hz, 3H), 0.72 (d, J = |
| | by SFC using AD-H | | | | 7.0 Hz, 3H) ppm. |
| | column; precursor | | | | |
| | was second eluting | | | | |
| | isomer by SFC on | | | | |
| | Lux Cellulose-2 | | | | |
| | column) | | | | |
| 113 | rel-(2R,3S,4S,5R)-N- | 535.527 | 536.5 | 3.37 | ¹H NMR (500 MHz, DMSO-d6) |
| | (2-(N,S- | | | | δ 10.87 (s, 1H), 8.60 (d, J = 5.5 |
| | dimethylsulfonimido- | | | | Hz, 1H), 8.33 (d, J = 2.1 Hz, |
| | yl)pyridin-4-yl)-3-(2- | | | | 1H), 7.81 (d, J = 4.9 Hz, 1H), |
| | ethoxy-3,4- | | | | 7.20-7.10 (m, 2H), 5.11 (d, J = |
| | difluorophenyl)-4,5- | | | | 10.4 Hz, 1H), 4.28 (dd, J = 10.4, |
| | dimethyl-5- | | | | 7.6 Hz, 1H), 4.23-4.11 (m, |
| | (trifluoromethyl)tetra- | | | | 2H), 3.14 (s, 3H), 2.75 (p, J = |
| | hydrofuran-2- | | | | 7.5 Hz, 1H), 2.45 (s, 3H), 1.61 |
| | carboxamide | | | | (s, 3H), 1.34 (t, J = 7.0 Hz, 3H), |
| | (second eluting | | | | 0.78-0.66 (m, 3H) ppm. |
| | isomer by SFC using | | | | |
| | AD-H column; | | | | |
| | precursor was second | | | | |
| | eluting isomer by | | | | |
| | SFC on Lux | | | | |
| | Cellulose-2 column) | | | | |

The following compounds were made using the method described in Example 3, except that 5-(methylthio)pyridin-3-amine was used in the amide coupling step 4. The purification in step was conducted by chiral SFC using a Lux Cellulose-2 column, (m particle size, 25 cm×10 mm from Phenomenex, Inc. on a Minigram SFC instrument from Berger Instruments and the first eluting isomer was subjected to General Method G followed by General Method H as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 114 | rel-3-((2R,3S,4S,5R)- | 536.535 | 536.1 | 3.77 | ¹H NMR (500 MHz, |
| | 3-(2-ethoxy-3,4- | | | | Chloroform-d) δ 10.37 (s, 1H), |
| | difluorophenyl)-4,5- | | | | 9.64 (s, 1H), 8.75 (s, 1H), 7.14 |
| | dimethyl-5- | | | | (t, J = 6.8 Hz, 1H), 6.87 (q, J = |
| | (trifluoromethyl)tetra- | | | | 8.9 Hz, 1H), 5.15 (d, J = 11.1 |
| | hydrofuran-2- | | | | Hz, 1H), 4.51 (s, 3H), 4.33 (dd, |
| | carboxamido)-1- | | | | J = 11.1, 7.8 Hz, 1H), 4.29- |
| | methyl-5-(S- | | | | 4.10 (m, 2H), 3.26 (s, 3H), 2.80 |
| | methylsulfonimidoyl)py- | | | | (p, J = 7.9 Hz, 1H), 1.75 (s, 3H), |
| | ridin-1-ium (TFA | | | | 1.43 (t, J = 7.0 Hz, 3H), 0.92- |
| | salt) | | | | 0.50 (m, 3H) ppm. |
| | (second eluting | | | | |
| | isomer by SFC using | | | | |
| | Whelk-O1 column; | | | | |
| | precursor was first | | | | |
| | eluting isomer by | | | | |
| | SFC on Lux | | | | |
| | Cellulose-2 column) | | | | |

The following compounds were made using the method described in Example 3 except 2-bromopropane was used as the alkylating agent in step 2, KOt-Bu in t-BuOH was used for epimerization/hydrolysis in step 3 and 4-methylsulfonylpyridin-2-amine was used as the amine in step 4. The purification in step 5 was conducted by chiral SFC using an (R,R)-Whelk-O 1 column, 5 m particle size, 25 cm×21.2 mm from Regis Technologies. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 115 | rel-(2S,3R,4R,5S)-3- | 536.512 | 537.1 | 3.62 | |
| | (3,4-difluoro-2- | | | | |
| | isopropoxyphenyl)- | | | | |
| | 4,5-dimethyl-N-(4- | | | | |
| | (methylsulfonyl)pyridin- | | | | |
| | 2-yl)-5- | | | | |
| | (trifluoromethyl)tetra- | | | | |
| | hydrofuran-2- | | | | |
| | carboxamide | | | | |
| | (First eluting peak by | | | | |
| | SFC) | | | | |
| 116 | rel-(2R,3S,4S,5R)-3- | 536.512 | 537.1 | 3.62 | ¹H NMR (500 MHz, DMSO- |
| | (3,4-difluoro-2- | | | | d₆) δ 10.65 (s, 1H), 8.66 (dd, J = |
| | isopropoxyphenyl)- | | | | 5.2, 0.8 Hz, 1H), 8.49 (dd, J = |
| | 4,5-dimethyl-N-(4- | | | | 1.7, 0.8 Hz, 1H), 7.66 (dd, J = |
| | (methylsulfonyl)pyridin- | | | | 5.1, 1.7 Hz, 1H), 7.24-7.12 |
| | 2-yl)-5- | | | | (m, 2H), 5.26 (d, J = 10.8 Hz, |
| | (trifluoromethyl)tetra- | | | | 1H), 4.57 (pd, J = 6.1, 1.2 Hz, |
| | hydrofuran-2- | | | | 1H), 4.33 (dd, J = 10.8, 7.3 Hz, |
| | carboxamide | | | | 1H), 3.28 (s, 3H), 2.75 (p, J = |
| | (Second eluting peak | | | | 7.4 Hz, 1H), 1.61 (s, 3H), 1.35 |
| | by SFC) | | | | (d, J = 6.1 Hz, 3H), 1.23 (d, J = |
| | | | | | 6.1 Hz, 3H), 0.75-0.64 (m, |
| | | | | | 3H) ppm. |

The following compounds were made using the method described in Example 3 except 2-bromopropane was used as the alkylating agent in step 2, KOt-Bu in t-BuOH was used for epimerization/hydrolysis in step 3 and 2-methylsulfanylpyridin-4-amine was used as amine in step 4. General Method G was used in place of step 5 and the 4 isomers generated were separated by chiral SFCs. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 117 | rel-(2S,3R,4R,5S)-3-(3,4- | 535.527 | 536.1 | 3.32 | |
| | difluoro-2-isopropoxyphenyl)- | | | | |
| | 4,5-dimethyl-N-(2-(S- | | | | |
| | methylsulfonimidoyl)pyridin- | | | | |
| | 4-yl)-5- | | | | |
| | (trifluoromethyl)tetrahydro- | | | | |
| | furan-2-carboxamide | | | | |
| | (First eluting peak by SFC on | | | | |
| | AS-H column then first eluting | | | | |
| | peak on AD-H column) | | | | |
| 118 | rel-(2S,3R,4R,5S)-3-(3,4- | 535.527 | 536.1 | 3.32 | |
| | difluoro-2-isopropoxyphenyl)- | | | | |
| | 4,5-dimethyl-N-(2-(S- | | | | |
| | methylsulfonimidoyl)pyridin- | | | | |
| | 4-yl)-5- | | | | |
| | (trifluoromethyl)tetrahydro- | | | | |
| | furan-2-carboxamide | | | | |
| | (First eluting peak by SFC on | | | | |
| | AS-H column then second | | | | |
| | eluting peak on AD-H column) | | | | |
| 119 | rel-(2R,3S,4S,5R)-3-(3,4- | 535.527 | 536.1 | 3.32 | |
| | difluoro-2-isopropoxyphenyl)- | | | | |
| | 4,5-dimethyl-N-(2-(S- | | | | |
| | methylsulfonimidoyl)pyridin- | | | | |
| | 4-yl)-5- | | | | |
| | (trifluoromethyl)tetrahydro- | | | | |
| | furan-2-carboxamide | | | | |
| | (Second eluting peak by SFC | | | | |
| | on AS-H column then first | | | | |
| | eluting peak on Whelk-O1 | | | | |
| | column) | | | | |
| 120 | rel-(2R,3S,4S,5R)-3-(3,4- | 535.527 | 536.1 | 3.32 | |
| | difluoro-2-isopropoxyphenyl)- | | | | |
| | 4,5-dimethyl-N-(2-(S- | | | | |
| | methylsulfonimidoyl)pyridin- | | | | |
| | 4-yl)-5- | | | | |
| | (trifluoromethyl)tetrahydro- | | | | |
| | furan-2-carboxamide | | | | |
| | (Second eluting peak by SFC | | | | |
| | on AS-H column then second | | | | |
| | eluting peak on Whelk-O1 | | | | |
| | column) | | | | |

### Example 4

rac-(2R,3S,4S,5R)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (121),
rel-(2S,3R,4R,5S)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (122) and
rel-(2R,3S,4S,5R)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (123)

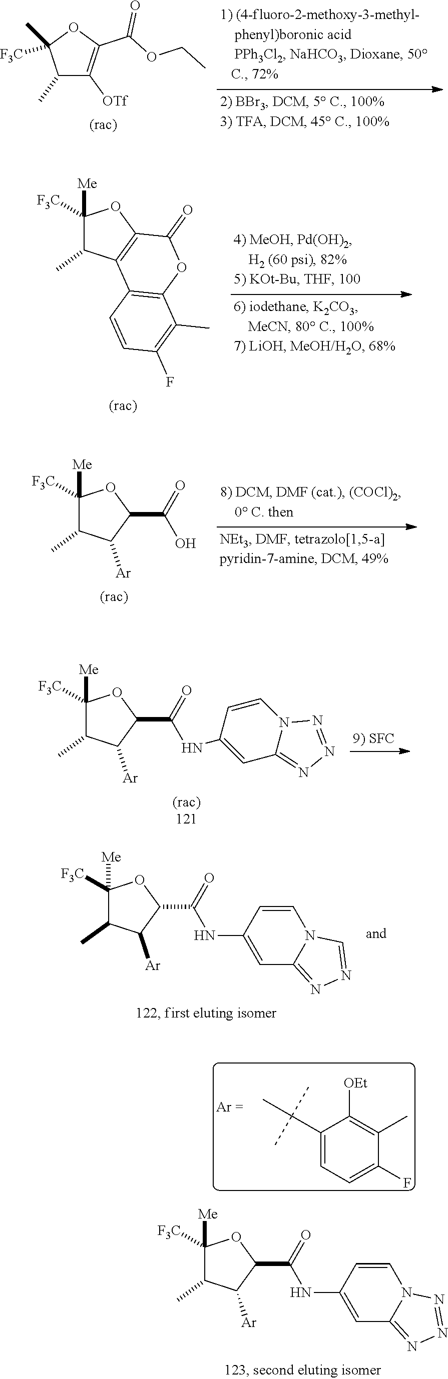

### Step 1

To a 2 L three necked round bottom flask flanked with a thermometer, was added a mixture of ethyl rac-(4R,5R)-4,5-dimethyl-5-(trifluoromethyl)-3-(((trifluoromethyl)sulfonyl)oxy)-4,5-dihydrofuran-2-carboxylate (39.05 g, 101.1 mmol), (4-fluoro-2-methoxy-3-methyl-phenyl)boronic acid (20.4 g, 110.9 mmol), PdCl₂(PPh₃)₂, (1.4 g, 1.995 mmol) and NaHCO₃ (120 mL) in 1,4-dioxane (400 mL). The orange mixture was heated slowly to 50° C. (internal temperature) and stirred for 20 minutes. The reaction mixture was cooled to ambient temperature and diluted with ethyl acetate (100 mL) and water (100 mL). The layers were separated and the aqueous phase was extracted with ethyl acetate (4×100 mL). The combined organic extracts were washed with brine (1×50 mL), dried (MgSO₄), filtered, and concentrated in vacuo to 100 mL. Charcoal (10 g) was added and reaction was mixture stirred for 2 hours. This mixture was filtered, washing through with ethyl acetate. The filtrate was concentrated in vacuo to give 50 g of crude product with no more solids. Purification by flash chromatography (330 g SiO₂, 0 to 35% ethyl acetate in heptane) gave ethyl rac-(4S,5R)-3-(4-fluoro-2-methoxy-3-methylphenyl)-4,5-dimethyl-5-(trifluoromethyl)-4,5-dihydrofuran-2-carboxylate (27.3 g, 72%) as a pale yellow oil. ¹H NMR (500 MHz, Chloroform-d) δ 6.98-6.88 (m, 1H), 6.81 (t, J=8.7 Hz, 1H), 4.20-4.07 (m, 2H), 3.66 (s, 3H), 3.58-3.49 (m, 1H), 2.21 (d, J=2.1 Hz, 3H), 1.7 (s, 3H), 1.12 (t, J=7.1 Hz, 3H), 1.06 (dq, J=7.2, 2.3 Hz, 3H) ppm. ESI-MS m/z calc. 376.12976, found 377.5 (M+1)⁺; Retention time: 1.09 minutes.

### Step 2

To a 1 L 3 neck flask flanked with a thermometer, was added ethyl rac-(4S,5R)-3-(4-fluoro-2-methoxy-3-methylphenyl)-4,5-dimethyl-5-(trifluoromethyl)-4,5-dihydrofuran-2-carboxylate (27.35 g, 72.67 mmol) followed by DCM (200 mL). This mixture was cooled to 5° C. in an ice bath. A solution of boron tribromide in DCM (112 mL of 1 M, 112.0 mmol) was added via cannular over 30 mins keeping temperature around 5° C. and the reaction mixture was left stirring for 1 hour. Upon completion, the mixture was quenched with water (very slowly as first few drops added caused reaction to effervesce) (100 mL). A saturated solution of NaHCO₃ (100 mL) was added and the mixture was stirred for 30 mins. The aqueous phase was extracted with DCM (3×50 mL) and the organic layer was washed with NaHCO₃ (5×100 mL). The combined organic layers were dried with MgSO₄, filtered and concentrated in vacuo. This solid was re-dissolved in ethyl acetate (100 mL) and charcoal (15 g) was added and allowed to stir at ambient temperature overnight. The reaction mixture was filtered through celite the and filtrate was concentrated in vacuo to give ethyl rac-(4S,5R)-3-(4-fluoro-2-hydroxy-3-methylphenyl)-4,5-dimethyl-5-(trifluoromethyl)-4,5-dihydrofuran-2-carboxylate (27.7 g, 100%) as a yellow waxy solid. ESI-MS m/z calc. 362.11414, found 363.5 (M+1)⁺; 361.5 (M−1)⁻; Retention time: 0.99 minutes.

### Step 3

TFA (9.8 mL, 127.2 mmol) was added to a solution of ethyl rac-(4S,5R)-3-(4-fluoro-2-hydroxy-3-methylphenyl)-4,5-dimethyl-5-(trifluoromethyl)-4,5-dihydrofuran-2-carboxylate (27.7 g, 76.45 mmol) in DCM (200 mL) at ambient temperature under stirring. The reaction mixture was heated at reflux and stirred at this temperature for 2.5 hours. The reaction mixture was cooled to ambient temperature and quenched with a saturated aqueous NaHCO₃ solution (100 mL) and the layers were separated. The DCM layer was washed with a saturated aqueous NaHCO₃ solution (4×100 mL). The organic extracts were dried (Na₂SO₄) and concentrated in vacuo to give a waxy solid. This solid was re-dissolved in ethyl acetate (200 mL). Activated charcoal (10 g) was added to the mixture which was allowed to stir at ambient temperature overnight. The mixture was filtered through a celite cartridge, washing with ethyl acetate (3×100 ml). The filtrate was concentrated in vacuo to give rac-(1S,2R)-7-fluoro-1,2,6-trimethyl-2-(trifluoromethyl)-1,2-dihydro-4H-furo[2,3-c]chromen-4-one (24.18 g, 100%) as a waxy solid. ESI-MS m/z calc. 316.07227, found 317.4 (M+1)⁺; 315.4 (M−1)⁻; Retention time: 0.94 minutes.

### Step 4:

rac-(1S,2R)-7-fluoro-1,2,6-trimethyl-2-(trifluoromethyl)-1,2-dihydro-4H-furo[2,3-c]chromen-4-one (1.5 g, 3.273 mmol) was dissolved in EtOAc (20 mL) and stirred with activated charcoal (300 mg, 24.98 mmol) for 18 hours. The solution was then filtered through celite and concentrated in-vacuo to give a yellow solid. This solid was then redissolved in methanol (20 mL) and added to a flask containing dihydroxypalladium (460 mg of 20% w/w, 0.655 mmol). The reaction mixture was evacuated and back filled with nitrogen (×3) then evacuated and back filled with hydrogen (×3) and finally left under a hydrogen balloon for 120 hours. The reaction mixture was filtered through a celite cartridge washing with MeOH. The mixture was then concentrated down to 20 mL and recharged to a flask with dihydroxypalladium (230 mg of 20% w/w, 0.3276 mmol). The reaction mixture was evacuated and back filled with nitrogen (×3) then evacuated and back filled with hydrogen (×3) and finally left under a hydrogen balloon for 12 hours. The reaction mixture was filtered through a celite cartridge washing with methanol and concentrated to give rac-methyl (2S,3S,4S,5R)-3-(4-fluoro-2-hydroxy-3-methylphenyl)-4,5-dimethyl-5-(trifluoromethyl)tetrahydrofuran-2-carboxylate (939.3 mg, 82%) as an off-white solid. ¹H NMR (500 MHz, Chloroform-d) δ 7.20 (t, J=7.7 Hz, 1H), 6.57 (t, J=8.9 Hz, 1H), 4.88 (d, J=6.1 Hz, 2H), 4.28 (dd, J=8.4, 6.1 Hz, 1H), 3.56 (s, 3H), 2.81 (p, J=7.8 Hz, 1H), 2.14 (d, J=1.6 Hz, 3H), 1.4 (3H-peak under water), 0.92 (dq, J=7.6, 1.9 Hz, 3H). ESI-MS m/z calc. 350.11414, found 349.0 (M−1)⁻; Retention time: 0.95 minutes.

### Step 5:

Potassium tert-butoxide (905 mg, 8.065 mmol) was added to a stirred solution of rac-methyl (2S,3S,4S,5R)-3-(4-fluoro-2-hydroxy-3-methylphenyl)-4,5-dimethyl-5-(trifluoromethyl)tetrahydrofuran-2-carboxylate (939.3 mg, 2.681 mmol) in THF (15 mL) and the reaction mixture stirred at ambient temperature for 5 minutes. 1 M HCl (3 mL) was added and the layers separated. The aqueous layer was extracted with DCM (3×5 mL) and the combined organic extracts dried (MgSO₄), filtered and concentrated in-vacuo to give rac-(2R,3S,4S,5R)-3-(4-fluoro-2-hydroxy-3-methylphenyl)-4,5-dimethyl-5-(trifluoromethyl)tetrahydrofuran-2-carboxylic acid (1.0268 g, 99%). ESI-MS m/z calc. 336.09848, found 335.0 (M−1)⁻; Retention time: 0.63 minutes.

### Step 6:

To a solution of rac-(2R,3S,4S,5R)-3-(4-fluoro-2-hydroxy-3-methylphenyl)-4,5-dimethyl-5-(trifluoromethyl)tetrahydrofuran-2-carboxylic acid

(513 mg, 1.327 mmol) in acetonitrile (3 mL) was added K₂CO₃ (735 mg, 5.318 mmol) and iodoethane (470 μL, 5.876 mmol). The reaction mixture was heated at 80° C. overnight in a sealed tube. The reaction was then diluted with DCM, filtered and the solid washed with DCM. The filtrate was carefully concentrated using a cold water bath to give ethyl rac-(2R,3S,4S,5R)-3-(2-ethoxy-4-fluoro-3-methylphenyl)-4,5-dimethyl-5-(trifluoromethyl)tetrahydrofuran-2-carboxylate (522.6 mg, 100%) as a yellow oil. ESI-MS m/z calc. 392.16107, found 393.5 (M+1)⁺; Retention time: 1.09 minutes.

### Step 7:

LiOH (1.4 mL of 2 M, 2.800 mmol) was added to a stirred solution of ethyl rac-(2R,3S,4S,5R)-3-(2-ethoxy-4-fluoro-3-methylphenyl)-4,5-dimethyl-5-(trifluoromethyl)tetrahydrofuran-2-carboxylate (522.6 mg, 1.332 mmol) in methanol (6 mL)/water (1 mL) and the mixture was stirred at ambient temperature for 1 hour. The reaction was concentrated in-vacuo and quenched with 1M HCl. The layers were separated and the aqueous layer extracted with DCM (2×5 mL). The combined organic extracts were dried by using a phase separation cartridge, filtered and concentrated in vacuo to give rac-(2R,3S,4S,5R)-3-(2-ethoxy-4-fluoro-3-methylphenyl)-4,5-dimethyl-5-(trifluoromethyl)tetrahydrofuran-2-carboxylic acid (330.9 mg, 68%). ¹H NMR (500 MHz, Chloroform-d) δ 7.09-6.99 (m, 1H), 6.81 (t, J=8.7 Hz, 1H), 4.90 (d, J=10.9 Hz, 1H), 4.13 (dd, J=11.0, 7.9 Hz, 1H), 3.92-3.83 (m, 1H), 3.77 (dq, J=9.6, 7.0 Hz, 1H), 2.71 (q, J=7.6 Hz, 1H), 2.24-2.13 (m, 3H), 1.62 (d, J=11.2 Hz, 3H), 1.47-1.34 (m, 3H), 0.75 (dq, J=4.7, 2.3 Hz, 3H). ESI-MS m/z calc. 364.12976, found 363.6 (M−1)⁻; Retention time: 0.62 minutes.

### Step 8:

A solution of rac-(2R,3S,4S,5R)-3-(2-ethoxy-4-fluoro-3-methylphenyl)-4,5-dimethyl-5-(trifluoromethyl)tetrahydrofuran-2-carboxylic acid (110 mg, 0.3019 mmol) in DCM (4 mL) was cooled using an ice-bath. To this was added DMF (13 μL, 0.1679 mmol) (1 drop DMF) followed by careful addition of oxalyl chloride (81 μL, 0.9285 mmol). The solution was stirred with ice-bath in place for 10 mins. The solution was concentrated in-vacuo and azeotroped with DCM to afford a yellow solid. This acid chloride was taken up in DCM (4 mL) and added to an ice bath cooled solution of tetrazolo[1,5-a]pyridin-7-amine (45 mg, 0.333 mmol) and DIPEA (260 μL, 1.493 mmol) in DCM (4 mL). The resulting dark suspension was stirred with ice-bath in place for 72 hours. The reaction mixture is partitioned with DCM and water. The layers were separated using a phase separation cartridge and the organics concentrated in-vacuo. The residue was purified by flash chromatography (4 g SiO₂, 0 to 100% EtOAc in heptane, loaded in DCM) to give rac-(2R,3S,4S,5R)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (121, 75.4 mg, 49%) as a white solid. ¹H NMR (500 MHz, DMSO-d₆) δ 10.98 (s, 1H), 9.21 (dd, J=7.5, 0.9 Hz, 1H), 8.46 (dd, J=2.1, 0.8 Hz, 1H), 7.46 (dd, J=7.5, 2.1 Hz, 1H), 7.24-7.17 (m, 1H), 6.98 (t, J=8.8 Hz, 1H), 5.11 (d, J=10.6 Hz, 1H), 4.35 (dd, J=10.6, 7.5 Hz, 1H), 3.86 (ddq, J=30.7, 9.3, 6.9 Hz, 2H), 2.74 (q, J=7.4 Hz, 1H), 2.15 (d, J=2.0 Hz, 3H), 1.63 (s, 3H), 1.40 (t, J=7.0 Hz, 3H), 0.74 (d, J=7.5 Hz, 3H). ESI-MS m/z calc. 481.1737, found 482.6 (M+1)⁺; 480.5 (M−1)⁻; Retention time: 3.75 minutes.

### Step 9:

Purification of rac-(2R,3S,4S,5R)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (122) (73.4 mg, 0.1448 mmol) by chiral SFC [System: (R,R)-Whelk-01 column, 5 μm particle size, 25 cm×21.2 mm from Regis Technologies] gave:

First eluting isomer (retention time=2.35 minutes): rel-(2S,3R,4R,5S)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (122, 23.80 mg, 62%). ¹H NMR (500 MHz, DMSO-d₆) δ 10.97 (s, 1H), 9.21 (dd, J=7.5, 0.8 Hz, 1H), 8.46 (dd, J=2.1, 0.9 Hz, 1H), 7.46 (dd, J=7.4, 2.1 Hz, 1H), 7.21 (dd, J=8.8, 6.4 Hz, 1H), 6.98 (t, J=8.8 Hz, 1H), 5.11 (d, J=10.7 Hz, 1H), 4.35 (dd, J=10.7, 7.4 Hz, 1H), 3.86 (ddq, J=30.4, 9.3, 7.0 Hz, 2H), 2.75 (p, J=7.4 Hz, 1H), 2.15 (d, J=2.0 Hz, 3H), 1.63 (s, 3H), 1.40 (t, J=7.0 Hz, 3H), 0.79-0.65 (m, 3H). ¹⁹F NMR (471 MHz, DMSO-d₆) δ−73.37, −115.75. ESI-MS m/z calc. 481.1737, found 482.6 (M+1); 480.6 (M−1)⁻; Retention time: 3.5 minutes.

Second eluting isomer (retention time=3.76 minutes): rel-(2R,3S,4S,5R)-3-(2-ethoxy-4-fluoro-3-methylphenyl)-4,5-dimethyl-N-(tetrazolo[1,5-a]pyridin-7-yl)-5-(trifluoromethyl)tetrahydrofuran-2-carboxamide (123, 18.96 mg, 54%) 1H NMR (500 MHz, DMSO-d6) δ 10.98 (s, 1H), 9.21 (dd, J=7.4, 0.8 Hz, 1H), 8.46 (dd, J=2.0, 0.8 Hz, 1H), 7.46 (dd, J=7.4, 2.1 Hz, 1H), 7.21 (dd, J=8.8, 6.5 Hz, 1H), 6.98 (t, J=8.8 Hz, 1H), 5.11 (d, J=10.6 Hz, 1H), 4.35 (dd, J=10.6, 7.5 Hz, 1H), 3.86 (ddq, J=30.2, 9.4, 6.9 Hz, 2H), 2.75 (p, J=7.4 Hz, 1H), 2.15 (d, J=2.0 Hz, 3H), 1.63 (s, 3H), 1.40 (t, J=7.0 Hz, 3H), 0.79-0.67 (i, 3H). 19 F NMR (47 1 MHz, DMSO-d 6) δ−73.37, −115.76. ESI-MS m/z calc. 481.1737, found 482.6 (M+1)⁺; 480.6 (M−1)⁻; Retention time: 3.51 minutes

The following compounds were made using the method described in Example 4, except that 2-methylsulfanylpyridin-4-amine (dihydrochloride salt) was used in the amide coupling step 8 and purification in step 9 SFC was conducted using chiral SFC using a Lux Cellulose-2 column, 5 m particle size, 25 cm×10 mm from Phenomenex, Inc. on a Minigram SFC instrument from Berger Instruments. The separated isomers from SFC were treated with conditions described in General Method G (step 1 only) as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 124 | rel-(2S,3R,4R,5S)-3- | 517.537 | 518.7 | 3.27 | ¹H NMR (500 MHz, DMSO- |
| | (2-ethoxy-4-fluoro-3- | | | | d₆) δ 10.93 (s, 1H), 8.57 (d, J = |
| | methylphenyl)-4,5- | | | | 5.5 Hz, 1H), 8.34 (dd, J = 6.5, |
| | dimethyl-N-(2-(S- | | | | 2.0 Hz, 1H), 7.80 (td, J = 5.7, |
| | methylsulfonimidoyl)py- | | | | 2.1 Hz, 1H), 7.20 (dd, J = 8.8, |
| | ridin-4-yl)-5- | | | | 6.5 Hz, 1H), 6.98 (t, J = 8.8 |
| | (trifluoromethyl)tetra- | | | | Hz, 1H), 5.08 (d, J = 10.7 Hz, |
| | hydrofuran-2- | | | | 1H), 4.38-4.25 (m, 2H), 3.85 |
| | carboxamide | | | | (ddq, J = 36.1, 9.3, 7.0 Hz, |
| | as a mixture of | | | | 2H), 3.12 (d, J = 1.0 Hz, 3H), |
| | epimers at the | | | | 2.74 (p, J = 7.5 Hz, 1H), 2.15 |
| | sulfonimidoyl position | | | | (d, J = 1.9 Hz, 3H), 1.62 (s, |
| | (precursor was first | | | | 3H), 1.39 (t, J = 7.0 Hz, 3H), |
| | eluting isomer by SFC | | | | 0.79-0.61 (m, 3H) ppm. |
| | on Lux Cellulose-2 | | | | |
| | column) | | | | |
| 125 | rel-(2R,3S,4S,5R)-3- | 517.537 | 518.6 | 3.3 | ¹H NMR (500 MHz, DMSO- |
| | (2-ethoxy-4-fluoro-3- | | | | d₆) δ 10.93 (s, 1H), 8.57 (d, J = |
| | methylphenyl)-4,5- | | | | 5.5 Hz, 1H), 8.34 (dd, J = 6.6, |
| | dimethyl-N-(2-(S- | | | | 2.0 Hz, 1H), 7.79 (td, J = 5.7, |
| | methylsulfonimidoyl)py- | | | | 2.1 Hz, 1H), 7.20 (dd, J = 8.7, |
| | ridin-4-yl)-5- | | | | 6.5 Hz, 1H), 6.98 (t, J = 8.9 |
| | (trifluoromethyl)tetra- | | | | Hz, 1H), 5.08 (d, J = 10.7 Hz, |
| | hydrofuran-2- | | | | 1H), 4.37-4.24 (m, 2H), 3.85 |
| | carboxamide | | | | (ddq, J = 36.2, 9.4, 7.0 Hz, |
| | as a mixture of | | | | 2H), 3.11 (d, J = 1.0 Hz, 3H), |
| | epimers at the | | | | 2.73 (q, J = 7.6 Hz, 1H), 2.15 |
| | sulfonimidoyl position | | | | (d, J = 1.9 Hz, 3H), 1.62 (s, |
| | (precursor was second | | | | 3H), 1.39 (t, J = 7.0 Hz, 3H), |
| | eluting isomer by SFC | | | | 0.80-0.63 (m, 3H) ppm. |
| | on Lux Cellulose-2 | | | | |
| | column) | | | | |

The following compounds were made using the method described in Example 4, except that 2-methylsulfanylpyridin-4-amine (dihydrochloride salt) was used in the amide coupling step 8 and purification in step 9 was conducted via chiral SFC using a Lux Cellulose-2 column, 5 μm particle size, 25 cm×10 mm from Phenomenex, Inc. on a Minigram SFC instrument from Berger Instruments. The separated isomers from SFC were treated with conditions described in General Method G as the final step. In the Table below, “MS r.t.” stands for Mass Spec retention time.

| | | LC/MS | | | NMR |
|---|---|---|---|---|---|
| Cmpd | | (m/z | Found | MS | (shifts |
| No. | Compound Name | calc.) | M + 1 | r.t. | in ppm) |
| 126 | rel-(2S,3R,4R,5S)-3-(2-ethoxy- | 517.537 | 518.5 | 3.3 | |
| | 4-fluoro-3-methylphenyl)-4,5- | | | | |
| | dimethyl-N-(2-(S- | | | | |
| | methylsulfonimidoyl)pyridin- | | | | |
| | 4-yl)-5- | | | | |
| | (trifluoromethyl)tetrahydrofura | | | | |
| | n-2-carboxamide | | | | |
| | (First eluting isomer by SFC | | | | |
| | on Whelk-O1 column; | | | | |
| | precursor came from first | | | | |
| | eluting peak by SFC on Lux | | | | |
| | Cellulose-2 column) | | | | |
| 127 | rel-(2S,3R,4R,5S)-3-(2-ethoxy- | 517.537 | 518.5 | 3.3 | |
| | 4-fluoro-3-methylphenyl)-4,5- | | | | |
| | dimethyl-N-(2-(S- | | | | |
| | methylsulfonimidoyl)pyridin- | | | | |
| | 4-yl)-5- | | | | |
| | (trifluoromethyl)tetrahydrofura | | | | |
| | n-2-carboxamide | | | | |
| | (Second eluting isomer by | | | | |
| | SFC on Whelk-O1 column; | | | | |
| | precursor came from first | | | | |
| | eluting peak by SFC on Lux | | | | |
| | Cellulose-2 column) | | | | |
| 128 | rel-(2R,3S,4S,5R)-3-(2-ethoxy- | 517.537 | 518.6 | 3.26 | |
| | 4-fluoro-3-methylphenyl)-4,5- | | | | |
| | dimethyl-N-(2-(S- | | | | |
| | methylsulfonimidoyl)pyridin- | | | | |
| | 4-yl)-5- | | | | |
| | (trifluoromethyl)tetrahydrofura | | | | |
| | n-2-carboxamide | | | | |
| | (First eluting isomer by SFC | | | | |
| | on AD-H column; precursor | | | | |
| | came from second eluting peak | | | | |
| | by SFC on Lux Cellulose-2 | | | | |
| | column) | | | | |
| 129 | rel-(2R,3S,4S,5R)-3-(2-ethoxy- | 517.537 | 518.6 | 3.26 | |
| | 4-fluoro-3-methylphenyl)-4,5- | | | | |
| | dimethyl-N-(2-(S- | | | | |
| | methylsulfonimidoyl)pyridin- | | | | |
| | 4-yl)-5- | | | | |
| | (trifluoromethyl)tetrahydrofura | | | | |
| | n-2-carboxamide | | | | |
| | (Second eluting isomer by | | | | |
| | SFC on AD-H column; | | | | |
| | precursor came from second | | | | |
| | eluting peak by SFC on Lux | | | | |
| | Cellulose-2 column) | | | | |

The following compounds were made using the method described in Example 4, except that 2-methylsulfanylpyridin-4-amine (dihydrochloride salt) was used in the amide coupling step 8 and purification by chiral SFC in step 9 SFC used a Lux Cellulose-2 column, 5 μm particle size, 25 cm×10 mm from Phenomenex, Inc. on a Minigram SFC instrument from Berger Instruments. The second eluting isomer from SFC was treated with conditions described in Step 1 of General Method G, then methylated using General Method H and finally chiral SFC using an (R,R)-Whelk-O 1 column, 5 μm particle size, 25 cm×21.2 mm from Regis Technologies was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 130 | rel-(2R,3S,4S,5R)-N-(2- | 531.563 | 532.2 | 3.38 | ¹H NMR (500 MHz, DMSO- |
| | (N,S- | | | | d₆) δ 10.90 (s, 1H), 8.59 (d, J = |
| | dimethylsulfonimidoyl) | | | | 5.5 Hz, 1H), 8.31 (d, J = |
| | pyridin-4-yl)-3-(2- | | | | 2.0 Hz, 1H), 7.78 (dd, J = |
| | ethoxy-4-fluoro-3- | | | | 5.7, 2.0 Hz, 1H), 7.19 (dd, J = |
| | methylphenyl)-4,5- | | | | 8.8, 6.5 Hz, 1H), 6.96 (t, J = |
| | dimethyl-5- | | | | 8.8 Hz, 1H), 5.06 (d, J = |
| | (trifluoromethyl)tetrahy | | | | 10.7 Hz, 1H), 4.29 (dd, J = |
| | drofuran-2-carboxamide | | | | 10.7, 7.5 Hz, 1H), 3.84 (ddq, |
| | (First eluting isomer by | | | | J = 34.5, 9.2, 6.9 Hz, 2H), |
| | SFC on Whelk-O1 | | | | 3.14 (s, 3H), 2.72 (p, J = 7.4 |
| | column; precursor came | | | | Hz, 1H), 2.44 (s, 3H), 2.14 |
| | from second eluting | | | | (d, J = 2.0 Hz, 3H), 1.61 (s, |
| | peak by SFC on Lux | | | | 3H), 1.37 (t, J = 7.0 Hz, 3H), |
| | Cellulose-2 column) | | | | 0.76-0.65 (m, 3H) ppm. |
| 131 | rel-(2R,3S,4S,5R)-N-(2- | 531.563 | 532.2 | 3.39 | ¹H NMR (500 MHz, DMSO- |
| | (N,S- | | | | d₆) δ 10.90 (s, 1H), 8.59 (d, J = |
| | dimethylsulfonimidoyl) | | | | 5.5 Hz, 1H), 8.31 (d, J = |
| | pyridin-4-yl)-3-(2- | | | | 2.0 Hz, 1H), 7.78 (dd, J = |
| | ethoxy-4-fluoro-3- | | | | 5.7, 2.0 Hz, 1H), 7.19 (dd, J = |
| | methylphenyl)-4,5- | | | | 8.8, 6.5 Hz, 1H), 6.96 (t, J = |
| | dimethyl-5- | | | | 8.8 Hz, 1H), 5.06 (d, J = |
| | (trifluoromethyl)tetrahy | | | | 10.7 Hz, 1H), 4.29 (dd, J = |
| | drofuran-2-carboxamide | | | | 10.7, 7.5 Hz, 1H), 3.84 (ddq, |
| | (Second eluting isomer | | | | J = 34.5, 9.2, 6.9 Hz, 2H), |
| | by SFC on Whelk-O1 | | | | 3.14 (s, 3H), 2.72 (p, J = 7.4 |
| | column; precursor came | | | | Hz, 1H), 2.44 (s, 3H), 2.14 |
| | from second eluting | | | | (d, J = 2.0 Hz, 3H), 1.61 (s, |
| | peak by SFC on Lux | | | | 3H), 1.37 (t, J = 7.0 Hz, 3H), |
| | Cellulose-2 column) | | | | 0.76-0.65 (m, 3H) ppm. |

The following compounds were made using the method described in Example 4, except that iodomethane was used in step 6. For 132 no final step SFC separation of isomers was carried out. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 132 | rac-(2R,3S,4S,5R)-3-(4- | 467.417 | 468.6 | 3.35 | ¹H NMR (500 MHz, DMSO- |
| | fluoro-2-methoxy-3- | | | | d₆) δ 10.94 (s, 1H), 9.20 (dd, |
| | methylphenyl)-4,5- | | | | J = 7.4, 0.9 Hz, 1H), 8.46 |
| | dimethyl-N- | | | | (dd, J = 2.1, 0.9 Hz, 1H), |
| | (tetrazolo[1,5-a]pyridin- | | | | 7.46 (dd, J = 7.4, 2.1 Hz, |
| | 7-yl)-5- | | | | 1H), 7.20 (dd, J = 8.7, 6.5 |
| | (trifluoromethyl)tetrahy | | | | Hz, 1H), 6.98 (t, J = 8.9 Hz, |
| | drofuran-2-carboxamide | | | | 1H), 5.10 (d, J = 10.4 Hz, |
| | | | | | 1H), 4.33 (dd, J = 10.5, 7.7 |
| | | | | | Hz, 1H), 3.73 (s, 3H), 2.75 |
| | | | | | (p, J = 7.6 Hz, 1H), 2.15 (d, J = |
| | | | | | 2.0 Hz, 3H), 1.62 (s, 3H), |
| | | | | | 0.78-0.71 (m, 3H) ppm. |
| 133 | rel-(2S,3R,4R,5S)-3-(4- | 467.417 | 468.6 | 3.35 | ¹H NMR (500 MHz, DMSO- |
| | fluoro-2-methoxy-3- | | | | d₆) δ 10.94 (s, 1H), 9.21 (dd, |
| | methylphenyl)-4,5- | | | | J = 7.5, 0.9 Hz, 1H), 8.46 |
| | dimethyl-N- | | | | (dd, J = 2.1, 0.9 Hz, 1H), |
| | (tetrazolo[1,5-a]pyridin- | | | | 7.47 (dd, J = 7.5, 2.1 Hz, |
| | 7-yl)-5- | | | | 1H), 7.21 (dd, J = 8.7, 6.5 |
| | (trifluoromethyl)tetrahy | | | | Hz, 1H), 6.99 (t, J = 8.9 Hz, |
| | drofuran-2-carboxamide | | | | 1H), 5.11 (d, J = 10.5 Hz, |
| | (First eluting isomer by | | | | 1H), 4.34 (dd, J = 10.5, 7.7 |
| | SFC using Whelk-O1 | | | | Hz, 1H), 3.74 (s, 3H), 2.75 |
| | column) | | | | (q, J = 7.6 Hz, 1H), 2.20- |
| | | | | | 2.09 (m, 3H), 1.63 (s, 3H), |
| | | | | | 0.79-0.67 (m, 3H) ppm. |
| 134 | rel-(2R,3S,4S,5R)-3-(4- | 467.417 | 468.6 | 3.35 | ¹H NMR (500 MHz, DMSO- |
| | fluoro-2-methoxy-3- | | | | d₆) δ 10.92 (s, 1H), 9.24- |
| | methylphenyl)-4,5- | | | | 9.14 (m, 1H), 8.46 (dd, J = |
| | dimethyl-N- | | | | 2.1, 0.8 Hz, 1H), 7.47 (dd, J = |
| | (tetrazolo[1,5-a]pyridin- | | | | 7.5, 2.1 Hz, 1H), 7.22 (dd, |
| | 7-yl)-5- | | | | J = 8.8, 6.5 Hz, 1H), 6.99 (t, |
| | (trifluoromethyl)tetrahy | | | | J = 8.8 Hz, 1H), 5.11 (d, J = |
| | drofuran-2-carboxamide | | | | 10.4 Hz, 1H), 4.34 (dd, J = |
| | (Second eluting isomer | | | | 10.5, 7.6 Hz, 1H), 3.74 (s, |
| | by SFC using Whelk-O1 | | | | 3H), 2.76 (p, J = 7.6 Hz, 1H), |
| | column) | | | | 2.16 (d, J = 2.0 Hz, 3H), 1.63 |
| | | | | | (s, 3H), 0.75 (dd, J = 7.6, 2.4 |
| | | | | | Hz, 3H) ppm. |

The following compounds were made using the method described in Example 4, except that iodomethane was used in step 6 and 2-(2,2-dimethyl-1,3-dioxolan-4-yl)pyridin-4-amine (first or second eluting isomer) was used in the amide coupling step 8. Purification by SFC in Step 9 used an (R,R)-Whelk-O 1 column, 5 μm particle size, 25 cm×21.2 mm from Regis Technologies and the separated isomers were then treated with General Method B as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 135 | rel-(2R,3S,4S,5R)-N-(2- | 486.457 | 487.6 | 3.04 | ¹H NMR (500 MHz, DMSO- |
| | (1,2- | | | | d₆) δ 10.87 (s, 1H), 8.43 (d, J = |
| | dihydroxyethyl)pyridin- | | | | 5.9 Hz, 1H), 7.88 (s, 1H), |
| | 4-yl)-3-(4-fluoro-2- | | | | 7.69 (s, 1H), 7.20 (dd, J = |
| | methoxy-3- | | | | 9.7, 7.4 Hz, 2H), 7.12 (s, |
| | methylphenyl)-4,5- | | | | 1H), 7.04-6.89 (m, 1H), |
| | dimethyl-5- | | | | 5.09 (d, J = 10.5 Hz, 1H), |
| | (trifluoromethyl)tetrahy | | | | 4.65 (s, 1H), 4.30 (dd, J = |
| | drofuran-2-carboxamide | | | | 10.6, 7.6 Hz, 1H), 3.73 (s, |
| | (precursor was second | | | | 3H), 3.63 (dd, J = 11.1, 4.6 |
| | eluting isomer by SFC | | | | Hz, 1H), 3.52 (dd, J = 11.1, |
| | on Whelk-O1 column; | | | | 6.2 Hz, 1H), 2.74 (p, J = 7.5 |
| | first eluting isomer of | | | | Hz, 1H), 2.16 (d, J = 1.9 Hz, |
| | amine was used) | | | | 3H), 1.62 (s, 3H), 0.78-0.63 |
| | | | | | (m, 3H) ppm. |
| 136 | rel-(2S,3R,4R,5S)-N-(2- | 486.457 | 487.6 | 3.04 | ¹H NMR (500 MHz, DMSO- |
| | (1,2- | | | | d₆) δ 10.56 (s, 1H), 8.33 (d, J = |
| | dihydroxyethyl)pyridin- | | | | 5.5 Hz, 1H), 7.69 (d, J = |
| | 4-yl)-3-(4-fluoro-2- | | | | 2.3 Hz, 1H), 7.49 (dd, J = |
| | methoxy-3- | | | | 5.5, 2.2 Hz, 1H), 7.20-7.14 |
| | methylphenyl)-4,5- | | | | (m, 1H), 6.98 (t, J = 8.8 Hz, |
| | dimethyl-5- | | | | 1H), 5.37 (d, J = 4.7 Hz, 1H), |
| | (trifluoromethyl)tetrahy | | | | 5.03 (d, J = 10.6 Hz, 1H), |
| | drofuran-2-carboxamide | | | | 4.64 (t, J = 5.9 Hz, 1H), 4.51 |
| | (precursor was first | | | | (dt, J = 7.3, 4.3 Hz, 1H), 4.28 |
| | eluting isomer by SFC | | | | (dd, J = 10.6, 7.6 Hz, 1H), |
| | on Whelk-O1 column; | | | | 3.72 (s, 3H), 3.64 (ddd, J = |
| | second eluting isomer of | | | | 10.3, 6.0, 4.1 Hz, 1H), 3.42 |
| | amine was used) | | | | (dt, J = 11.1, 6.3 Hz, 1H), |
| | | | | | 2.72 (p, J = 7.5 Hz, 1H), 2.14 |
| | | | | | (d, J = 1.9 Hz, 3H), 1.60 (s, |
| | | | | | 3H), 0.72 (d, J = 7.3 Hz, 3H) |
| | | | | | ppm. |
| 137 | rel-(2R,3S,4S,5R)-N-(2- | 486.457 | 487.6 | 3.05 | ¹H NMR (500 MHz, |
| | (1,2- | | | | Methanol-d₄) δ 8.36-8.30 |
| | dihydroxyethyl)pyridin- | | | | (m, 1H), 7.78 (d, J = 2.1 Hz, |
| | 4-yl)-3-(4-fluoro-2- | | | | 1H), 7.61 (dd, J = 5.7, 2.1 |
| | methoxy-3- | | | | Hz, 1H), 7.19 (dd, J = 8.7, |
| | methylphenyl)-4,5- | | | | 6.3 Hz, 1H), 6.86 (t, J = 8.8 |
| | dimethyl-5- | | | | Hz, 1H), 5.03 (d, J = 10.6 |
| | (trifluoromethyl)tetrahy | | | | Hz, 1H), 4.70 (dd, J = 6.7, |
| | drofuran-2-carboxamide | | | | 4.0 Hz, 1H), 4.35 (dd, J = |
| | (precursor was second | | | | 10.7, 7.9 Hz, 1H), 3.81-3.77 |
| | eluting isomer by SFC | | | | (m, 1H), 3.76 (s, 3H), 3.65 |
| | on Whelk-O1 column; | | | | (dd, J = 11.3, 6.6 Hz, 1H), |
| | second eluting isomer of | | | | 2.76 (p, J = 7.7 Hz, 1H), 2.20 |
| | amine was used) | | | | (d, J = 2.0 Hz, 3H), 1.66 (d, J = |
| | | | | | 1.2 Hz, 3H), 0.81 (dq, J = |
| | | | | | 7.4, 2.3 Hz, 3H) ppm. |

The following compound was made using the method described in Example 4, except that iodomethane was used in step 6 and rac-2-(2,2-dimethyl-1,3-dioxolan-4-yl)pyridin-4-amine was used in the amide coupling step 8. The first eluting isomer (major) from flash chromatography (SiO₂, 0 to 30% o EtOAc in heptanes) in step 8 was further purified by chiral SFC in step 9 using an (R,R)-Whelk-O 1 column, 5 μm particle size, 25 cm×21.2 mm from Regis Technologies. The first eluting isomer from the SFC was treated with General Method A and then General Method B as the final steps. In the Table below, “MS r.t.” stands for Mass Spec retention time.

| | | LC/MS | | | NMR |
|---|---|---|---|---|---|
| Cmpd | | (m/z | Found | MS | (shifts |
| No. | Compound Name | calc.) | M + 1 | r.t. | in ppm) |
| 138 | rel-2-(1,2-dihydroxyethyl)-4- | 502.456 | 503.7 | 2.89 | |
| | ((2S,3R,4R,5S)-3-(4-fluoro-2- | | | | |
| | methoxy-3-methylphenyl)-4,5- | | | | |
| | dimethyl-5- | | | | |
| | (trifluoromethyl)tetrahydrofuran- | | | | |
| | 2-carboxamido)pyridine 1- | | | | |
| | oxide | | | | |
| | (precursor was first eluting | | | | |
| | isomer by SFC) | | | | |

The following compound was made using the method described in Example 4, except that iodomethane was used in step 6 and 2-(2,2-dimethyl-1,3-dioxolan-4-yl)-5-fluoro-pyridin-4-amine (first or second eluting isomer) was used in the amide coupling step 8. Purification by SFC in step 9 used an (R,R)-Whelk-O 1 column, Sm particle size, 25 cm×21.2 mm from Regis Technologies and the separated isomers were then deprotected using General Method B as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 139 | rel-(2S,3R,4R,5S)-N-(2-(1,2- | 504.447 | 505.6 | 3.18 | ¹H NMR (500 MHz, |
| | dihydroxyethyl)-5- | | | | DMSO-d₆) δ 10.25 (s, |
| | fluoropyridin-4-yl)-3-(4- | | | | 1H), 8.43 (d, J = 2.4 Hz, |
| | fluoro-2-methoxy-3- | | | | 1H), 8.17 (d, J = 6.4 Hz, |
| | methylphenyl)-4,5-dimethyl- | | | | 1H), 7.26-7.17 (m, |
| | 5- | | | | 2H), 7.08 (s, 1H), 7.03- |
| | (trifluoromethyl)tetrahydrofuran- | | | | 6.92 (m, 2H), 5.30 (d, J = |
| | 2-carboxamide | | | | 10.7 Hz, 1H), 4.50 |
| | (precursor was first eluting | | | | (dd, J = 6.7, 4.1 Hz, |
| | isomer by SFC on Whelk-O1 | | | | 1H), 4.27 (dd, J = 10.8, |
| | column; first eluting isomer | | | | 7.4 Hz, 1H), 3.71 (s, |
| | of amine was used) | | | | 3H), 3.61 (dd, J = 11.0, |
| | | | | | 4.1 Hz, 1H), 2.73 (q, J = |
| | | | | | 7.4 Hz, 1H), 2.14 (d, J = |
| | | | | | 2.0 Hz, 3H), 1.61 (s, |
| | | | | | 3H), 0.71 (d, J = 7.2 Hz, |
| | | | | | 3H) ppm. |
| | rel-(2R,3S,4S,5R)-N-(2-(1,2- | 504.447 | 505.6 | 3.18 | |
| | dihydroxyethyl)-5- | | | | |
| | fluoropyridin-4-yl)-3-(4- | | | | |
| | fluoro-2-methoxy-3- | | | | |
| | methylphenyl)-4,5-dimethyl- | | | | |
| | 5- | | | | |
| | (trifluoromethyl)tetrahydrofuran- | | | | |
| | 2-carboxamide | | | | |
| | (precursor was second | | | | |
| | eluting isomer by SFC on | | | | |
| | Whelk-O1 column; first | | | | |
| | eluting isomer of amine was | | | | |
| | used) | | | | |
| 141 | rel-(2R,3S,4S,5R)-N-(2-(1,2- | 504.447 | 505.6 | 3.18 | ¹H NMR (500 MHz, |
| | dihydroxyethyl)-5- | | | | DMSO-d₆) δ 10.26 (s, |
| | fluoropyridin-4-yl)-3-(4- | | | | 1H), 8.44 (d, J = 2.3 Hz, |
| | fluoro-2-methoxy-3- | | | | 1H), 8.18 (d, J = 6.5 Hz, |
| | methylphenyl)-4,5-dimethyl- | | | | 1H), 7.25 (dd, J = 8.7, |
| | 5- | | | | 6.5 Hz, 1H), 6.99 (t, J = |
| | (trifluoromethyl)tetrahydrofu | | | | 8.8 Hz, 1H), 5.42 (d, J = |
| | ran-2-carboxamide | | | | 4.8 Hz, 1H), 5.31 (d, J = |
| | (precursor was second | | | | 10.7 Hz, 1H), 4.64 (t, J = |
| | eluting isomer by SFC on | | | | 5.9 Hz, 1H), 4.51 (dt, |
| | Whelk-O1 column; second | | | | J = 6.6, 4.5 Hz, 1H), |
| | eluting isomer of amine was | | | | 4.28 (dd, J = 10.7, 7.5 |
| | used) | | | | Hz, 1H), 3.73 (s, 3H), |
| | | | | | 3.62 (ddd, J = 10.4, 6.0, |
| | | | | | 4.1 Hz, 1H), 3.47-3.37 |
| | | | | | (m, 1H), 2.73 (p, J = 7.4 |
| | | | | | Hz, 1H), 2.16 (d, J = 2.0 |
| | | | | | Hz, 3H), 1.62 (s, 3H), |
| | | | | | 0.77-0.68 (m, 3H) |
| | | | | | ppm. |

The following compounds were made using the method described in Example 4, except that iodomethane was used in step 6 and 2-(2,2-dimethyl-1,3-dioxolan-4-yl)-5-fluoro-pyridin-4-amine (syn or anti diol) was used in the amide coupling step 8. The separated isomers from step 9 were treated with General Method B as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 142 | rel-(2S,3R,4R,5S)-N-(2-(syn- | 500.483 | 501.5 | 3.15 | ¹H NMR (500 MHz, |
| | 1,2-dihydroxypropyl)pyridin- | | | | DMSO-d₆) δ 10.60 (s, |
| | 4-yl)-3-(4-fluoro-2-methoxy- | | | | 1H), 8.35 (d, J = 5.6 Hz, |
| | 3-methylphenyl)-4,5- | | | | 1H), 7.77-7.62 (m, 1H), |
| | dimethyl-5- | | | | 7.57-7.47 (m, 1H), 7.18 |
| | (trifluoromethyl)tetrahydrofuran- | | | | (dd, J = 8.7, 6.4 Hz, 1H), |
| | 2-carboxamide | | | | 6.99 (t, J = 8.8 Hz, 1H), |
| | (precursor was first eluting | | | | 5.29 (s, 1H), 5.05 (d, J = |
| | isomer by SFC on Whelk-O1 | | | | 10.6 Hz, 1H), 4.48 (s, |
| | column and then first eluting | | | | 1H), 4.36-4.23 (m, 2H), |
| | isomer on Lux i-cellulose-5 | | | | 3.81 (s, 1H), 3.73 (s, 3H), |
| | column; syn diol was used to | | | | 2.73 (p, J = 7.4 Hz, 1H), |
| | make amine) | | | | 2.16 (d, J = 1.9 Hz, 3H), |
| | | | | | 1.61 (s, 3H), 0.97 (d, J = |
| | | | | | 6.4 Hz, 3H), 0.79-0.67 |
| | | | | | (m, 3H) ppm. |
| 143 | rel-(2S,3R,4R,5S)-N-(2-(syn- | 500.483 | 502.4 | 3.15 | ¹H NMR (500 MHz, |
| | 1,2-dihydroxypropyl)pyridin- | | | | DMSO-d₆) δ 10.58 (s, |
| | 4-yl)-3-(4-fluoro-2-methoxy- | | | | 1H), 8.34 (d, J = 5.5 Hz, |
| | 3-methylphenyl)-4,5- | | | | 1H), 7.66 (d, J = 2.1 Hz, |
| | dimethyl-5- | | | | 1H), 7.52 (dd, J = 5.6, 2.1 |
| | (trifluoromethyl)tetrahydrofu | | | | Hz, 1H), 7.18 (dd, J = 8.8, |
| | ran-2-carboxamide | | | | 6.5 Hz, 1H), 6.98 (t, J = |
| | (precursor was first eluting | | | | 8.8 Hz, 1H), 5.26 (d, J = |
| | isomer by SFC on Whelk-O1 | | | | 5.2 Hz, 1H), 5.05 (d, J = |
| | column and second on Lux i- | | | | 10.7 Hz, 1H), 4.46 (d, J = |
| | cellulose-5 column; syn diol | | | | 5.8 Hz, 1H), 4.35-4.20 |
| | was used to make amine) | | | | (m, 2H), 3.89-3.77 (m, |
| | | | | | 1H), 3.72 (s, 3H), 2.73 (p, |
| | | | | | J = 7.5 Hz, 1H), 2.15 (d, J = |
| | | | | | 2.2 Hz, 3H), 1.61 (s, |
| | | | | | 3H), 0.97 (d, J = 6.4 Hz, |
| | | | | | 3H), 0.80-0.62 (m, 3H) |
| | | | | | ppm. |
| 144 | rel-(2R,3S,4S,5R)-N-(2-(syn- | 500.483 | 502.7 | 3.16 | ¹H NMR (500 MHz, |
| | 1,2-dihydroxypropyl)pyridin- | | | | DMSO-d₆) δ 10.56 (s, |
| | 4-yl)-3-(4-fluoro-2-methoxy- | | | | 1H), 8.32 (d, J = 5.5 Hz, |
| | 3-methylphenyl)-4,5- | | | | 1H), 7.65 (d, J = 2.1 Hz, |
| | dimethyl-5- | | | | 1H), 7.50 (dd, J = 5.6, 2.2 |
| | (trifluoromethyl)tetrahydrofuran- | | | | Hz, 1H), 7.17 (dd, J = 8.7, |
| | 2-carboxamide | | | | 6.5 Hz, 1H), 6.97 (t, J = |
| | (precursor was second | | | | 8.8 Hz, 1H), 5.24 (d, J = |
| | eluting isomer by SFC on | | | | 5.2 Hz, 1H), 5.04 (d, J = |
| | Whelk-O1 column; syn diol | | | | 10.6 Hz, 1H), 4.45 (d, J = |
| | was used to make amine) | | | | 5.7 Hz, 1H), 4.34-4.24 |
| | | | | | (m, 2H), 3.85-3.74 (m, |
| | | | | | 1H), 3.72 (s, 3H), 2.73 (q, |
| | | | | | J = 7.5 Hz, 1H), 2.14 (d, J |
| | | | | | = 2.0 Hz, 3H), 1.60 (s, |
| | | | | | 3H), 0.95 (d, J = 6.3 Hz, |
| | | | | | 3H), 0.77-0.64 (m, 3H) |
| | | | | | ppm. |
| 145 | rel-(2R,3S,4S,5R)-N-(2-(syn- | 500.483 | 501.6 | 3.15 | ¹H NMR (500 MHz, |
| | 1,2-dihydroxypropyl)pyridin- | | | | DMSO-d₆) δ 10.87 (s, |
| | 4-yl)-3-(4-fluoro-2-methoxy- | | | | 1H), 8.40 (d, J = 6.0 Hz, |
| | 3-methylphenyl)-4,5- | | | | 1H), 7.76 (d, J = 73.1 Hz, |
| | dimethyl-5- | | | | 2H), 7.18 (dd, J = 8.7, 6.4 |
| | (trifluoromethyl)tetrahydrofuran- | | | | Hz, 1H), 6.97 (t, J = 8.8 |
| | 2-carboxamide | | | | Hz, 1H), 5.07 (d, J = 10.6 |
| | (precursor was third eluting | | | | Hz, 1H), 4.46 (s, 1H), |
| | isomer by SFC on Whelk-O1 | | | | 4.29 (dd, J = 10.6, 7.6 Hz, |
| | column; syn diol was used to | | | | 1H), 3.84 (dd, J = 6.6, 4.6 |
| | make amine) | | | | Hz, 1H), 3.71 (s, 3H), |
| | | | | | 2.73 (p, J = 7.5 Hz, 1H), |
| | | | | | 2.14 (d, J = 2.0 Hz, 3H), |
| | | | | | 1.60 (s, 3H), 0.97 (d, J = |
| | | | | | 6.4 Hz, 3H), 0.72 (d, J = |
| | | | | | 7.4 Hz, 3H) ppm. |
| 146 | rel-(2S,3R,4R,5S)-N-(2-(anti- | 500.483 | 501.5 | 3.16 | ¹H NMR (500 MHz, |
| | 1,2-dihydroxypropyl)pyridin- | | | | DMSO-d₆) δ 10.63 (s, |
| | 4-yl)-3-(4-fluoro-2-methoxy- | | | | 1H), 8.35 (d, J = 5.7 Hz, |
| | 3-methylphenyl)-4,5- | | | | 1H), 7.70 (s, 1H), 7.56 (d, |
| | dimethyl-5- | | | | J = 5.6 Hz, 1H), 7.18 (dd, |
| | (trifluoromethyl)tetrahydrofuran- | | | | J = 8.7, 6.5 Hz, 1H), 6.99 |
| | 2-carboxamide | | | | (t, J = 8.8 Hz, 1H), 5.35 |
| | (precursor was first eluting | | | | (s, 1H), 5.05 (d, J = 10.6 |
| | isomer by SFC on chiralpak | | | | Hz, 1H), 4.68 (s, 1H), |
| | IC column; anti diol was | | | | 4.42 (s, 1H), 4.30 (dd, J = |
| | used to make amine) | | | | 10.7, 7.6 Hz, 1H), 3.92- |
| | | | | | 3.82 (m, 1H), 3.73 (s, |
| | | | | | 3H), 2.74 (p, J = 7.5 Hz, |
| | | | | | 1H), 2.16 (d, J = 1.9 Hz, |
| | | | | | 3H), 1.61 (s, 3H), 0.94 (d, |
| | | | | | J = 6.3 Hz, 3H), 0.81- |
| | | | | | 0.67 (m, 3H) ppm. |
| 147 | rel-(2R,3S,4S,5R)-N-(2-(anti- | 500.483 | 502.6 | 3.16 | ¹H NMR (500 MHz, |
| | 1,2- | | | | DMSO-d₆) δ 10.80 (s, |
| | dihydroxypropyl)pyridin-4- | | | | 1H), 8.40 (s, 1H), 7.82 (s, |
| | yl)-3-(4-fluoro-2-methoxy-3- | | | | 1H), 7.65 (s, 1H), 7.18 |
| | methylphenyl)-4,5-dimethyl- | | | | (dd, J = 8.8, 6.3 Hz, 1H), |
| | 5- | | | | 6.97 (t, J = 8.9 Hz, 1H), |
| | (trifluoromethyl)tetrahydrofuran- | | | | 5.06 (d, J = 10.6 Hz, 1H), |
| | 2-carboxamide | | | | 4.43 (d, J = 5.3 Hz, 1H), |
| | (precursor was second | | | | 4.29 (dd, J = 10.7, 7.6 Hz, |
| | eluting isomer by SFC on | | | | 1H), 3.81 (s, 1H), 3.71 (s, |
| | chiralpak IC column; anti | | | | 3H), 2.73 (p, J = 7.5 Hz, |
| | diol was used to make | | | | 1H), 2.14 (d, J = 2.0 Hz, |
| | amine) | | | | 3H), 1.60 (s, 3H), 0.97 (d, |
| | | | | | J = 6.3 Hz, 3H), 0.72 (d, J = |
| | | | | | 7.1 Hz, 3H) ppm. |
| 148 | rel-(2R,3S,4S,5R)-N-(2-(anti- | 500.483 | 501.6 | 3.15 | ¹H NMR (500 MHz, |
| | 1,2-dihydroxypropyl)pyridin- | | | | DMSO-d₆) δ 10.57 (s, |
| | 4-yl)-3-(4-fluoro-2-methoxy- | | | | 1H), 8.32 (d, J = 5.6 Hz, |
| | 3-methylphenyl)-4,5- | | | | 1H), 7.65 (d, J = 2.1 Hz, |
| | dimethyl-5- | | | | 1H), 7.51 (dt, J = 5.2, 2.6 |
| | (trifluoromethyl)tetrahydrofuran- | | | | Hz, 1H), 7.17 (dd, J = 8.7, |
| | 2-carboxamide | | | | 6.5 Hz, 1H), 6.98 (t, J = |
| | (precursor was third eluting | | | | 8.9 Hz, 1H), 5.26 (d, J = |
| | isomer by SFC on chiralpak | | | | 4.8 Hz, 1H), 5.03 (d, J = |
| | IC column; anti diol was | | | | 10.6 Hz, 1H), 4.64 (d, J = |
| | used to make amine) | | | | 5.2 Hz, 1H), 4.40 (t, J = |
| | | | | | 4.8 Hz, 1H), 4.28 (dd, J = |
| | | | | | 10.6, 7.6 Hz, 1H), 3.87 (q, |
| | | | | | J = 5.3 Hz, 1H), 3.72 (s, |
| | | | | | 3H), 2.14 (d, J = 1.9 Hz, |
| | | | | | 3H), 1.60 (s, 3H), 0.91 (d, |
| | | | | | J = 6.3 Hz, 3H), 0.77- |
| | | | | | 0.63 (m, 3H) ppm. |
| 149 | rel-(2S,3R,4R,5S)-N-(2-(anti- | 500.483 | 501.6 | 3.15 | ¹H NMR (500 MHz, |
| | 1,2-dihydroxypropyl)pyridin- | | | | DMSO-d₆) δ 10.59 (s, |
| | 4-yl)-3-(4-fluoro-2-methoxy- | | | | 1H), 8.34 (d, J = 5.6 Hz, |
| | 3-methylphenyl)-4,5- | | | | 1H), 7.69 (d, J = 2.1 Hz, |
| | dimethyl-5- | | | | 1H), 7.50 (dd, J = 5.6, 2.1 |
| | (trifluoromethyl)tetrahydrofuran- | | | | Hz, 1H), 7.18 (dd, J = 8.7, |
| | 2-carboxamide | | | | 6.5 Hz, 1H), 6.99 (t, J = |
| | (precursor was fourth eluting | | | | 8.8 Hz, 1H), 5.28 (d, J = |
| | isomer by SFC on chiralpak | | | | 4.9 Hz, 1H), 5.05 (d, J = |
| | IC column; anti diol was | | | | 10.7 Hz, 1H), 4.65 (d, J = |
| | used to make amine) | | | | 5.2 Hz, 1H), 4.41 (t, J = |
| | | | | | 4.8 Hz, 1H), 4.30 (dd, J = |
| | | | | | 10.7, 7.6 Hz, 1H), 3.88 (h, |
| | | | | | J = 6.0 Hz, 1H), 3.73 (s, |
| | | | | | 3H), 2.73 (p, J = 7.5 Hz, |
| | | | | | 1H), 2.16 (d, J = 2.0 Hz, |
| | | | | | 3H), 1.61 (s, 3H), 0.92 (d, |
| | | | | | J = 6.3 Hz, 3H), 0.78- |
| | | | | | 0.65 (m, 3H) ppm. |

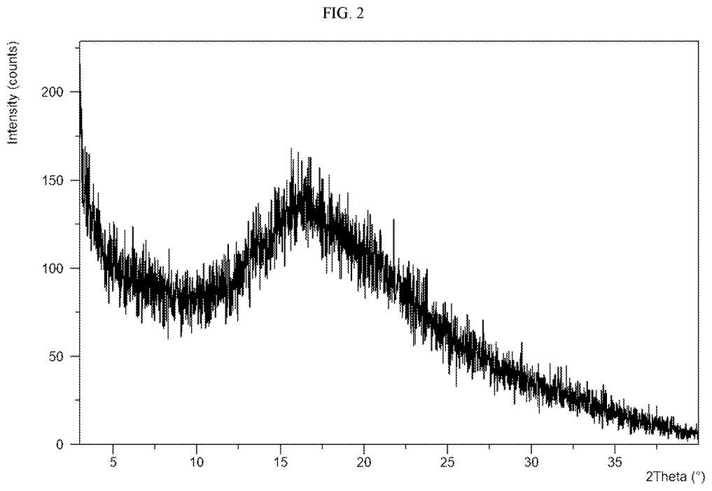

Compound 145 was analyzed by X-ray powder diffraction and determined to be amorphous (see FIG. 2).

The following compounds were made using the method described in Example 4, except that iodomethane was used in step 6 and 2-(methylthio)pyridin-4-amine was used in the amide coupling step 8. Purification by chiral SFC in step 9 used a Lux Cellulose-2 column, 5 μm particle size, 25 cm×10 mm from Phenomenex, Inc. on a Minigram SFC instrument from Berger Instruments. Step 1 of General Method G was used as the final step on separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 150 | rel-(2S,3R,4R,5S)-3-(4- | 503.51 | 504.1 | 3.12 | ¹H NMR (500 MHz, DMSO- |
| | fluoro-2-methoxy-3- | | | | d₆) δ 10.90 (s, 1H), 8.57 (d, J = |
| | methylphenyl)-4,5- | | | | 5.4 Hz, 1H), 8.35 (dd, J = |
| | dimethyl-N-(2-(S- | | | | 5.7, 2.1 Hz, 1H), 7.81 (td, J = |
| | methylsulfonimidoyl) | | | | 5.2, 2.1 Hz, 1H), 7.20 (dd, J = |
| | pyridin-4-yl)-5- | | | | 8.7, 6.5 Hz, 1H), 6.99 (t, J = |
| | (trifluoromethyl)tetrahy | | | | 8.8 Hz, 1H), 5.07 (d, J = |
| | drofuran-2-carboxamide | | | | 10.6 Hz, 1H), 4.36-4.19 (m, |
| | as a mixture of epimers | | | | 2H), 3.73 (s, 3H), 3.11 (d, J = |
| | at the sulfonimidoyl | | | | 1.0 Hz, 3H), 2.75 (p, J = 7.5 |
| | position | | | | Hz, 1H), 2.16 (d, J = 2.0 Hz, |
| | (precursor was first | | | | 3H), 1.63 (s, 3H), 0.74 (d, J = |
| | eluting isomer by SFC) | | | | 7.3 Hz, 3H) ppm. |
| 151 | rel-(2R,3S,4S,5R)-3-(4- | 503.51 | 504.1 | 3.12 | ¹H NMR (500 MHz, DMSO- |
| | fluoro-2-methoxy-3- | | | | d₆) δ 10.90 (s, 1H), 8.57 (d, J = |
| | methylphenyl)-4,5- | | | | 5.5 Hz, 1H), 8.34 (dd, J = |
| | dimethyl-N-(2-(S- | | | | 5.6, 2.0 Hz, 1H), 7.80 (td, J = |
| | methylsulfonimidoyl) | | | | 5.2, 2.1 Hz, 1H), 7.20 (dd, J = |
| | pyridin-4-yl)-5- | | | | 8.8, 6.5 Hz, 1H), 6.99 (t, J = |
| | (trifluoromethyl)tetrahy | | | | 8.8 Hz, 1H), 5.07 (d, J = |
| | drofuran-2-carboxamide | | | | 10.6 Hz, 1H), 4.37-4.23 (m, |
| | as a mixture of epimers | | | | 2H), 3.72 (s, 3H), 3.11 (d, J = |
| | at the sulfonimidoyl | | | | 1.1 Hz, 3H), 2.74 (p, J = 7.4 |
| | position | | | | Hz, 1H), 2.15 (d, J = 2.0 Hz, |
| | (precursor was second | | | | 3H), 1.62 (s, 3H), 0.73 (d, J = |
| | eluting isomer by SFC) | | | | 7.1 Hz, 3H) ppm. |

The following compounds were made using the method described in Example 4, except that iodomethane was used in step 6 and 2-(methylthio)pyridin-4-amine was used in the amide coupling step 8. Purification by chiral SFC in step 9 used a Lux Cellulose-2 column, 5 μm particle size, 25 cm×10 mm from Phenomenex, Inc. on a Minigram SFC instrument from Berger Instruments. Step 1 of General Method G, then methylation using General Method H and finally SFC using a (R,R)-Whelk-O1 column, Sum particle size, 25 cm×21.2 mm from Regis Technologies were used as the final steps on the second eluting isomer from step 9. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 152 | rel-(2R,3S,4S,5R)-N-(2- | 517.537 | 518.1 | 3.23 | ¹H NMR (500 MHz, DMSO- |
| | (N,S- | | | | d₆) δ 10.80 (s, 1H), 8.57 (d, J = |
| | dimethylsulfonimidoyl) | | | | 5.6 Hz, 1H), 8.30 (d, J = 2.0 |
| | pyridin-4-yl)-3-(4- | | | | Hz, 1H), 7.76 (s, 1H), 7.19 |
| | fluoro-2-methoxy-3- | | | | (dd, J = 8.7, 6.5 Hz, 1H), 6.97 |
| | methylphenyl)-4,5- | | | | (t, J = 8.8 Hz, 1H), 5.04 (d, J = |
| | dimethyl-5- | | | | 10.6 Hz, 1H), 4.28 (dd, J = |
| | (trifluoromethyl)tetrahy | | | | 10.6, 7.6 Hz, 1H), 3.71 (s, 3H), |
| | drofuran-2-carboxamide | | | | 3.13 (s, 3H), 2.72 (p, J = 7.5 |
| | (first eluting isomer by | | | | Hz, 1H), 2.44 (s, 3H), 2.14 (d, |
| | SFC on Whelk-O1 | | | | J = 2.0 Hz, 3H), 1.61 (s, 3H), |
| | column; precursor was | | | | 0.72 (d, J = 7.2 Hz, 3H) ppm. |
| | second eluting isomer | | | | |
| | by SFC on Lux | | | | |
| | cellulose-2 column) | | | | |
| 153 | rel-(2R,3S,4S,5R)-N-(2- | 517.537 | 518.1 | 3.23 | ¹H NMR (500 MHz, DMSO- |
| | (N,S- | | | | d₆) δ 8.59 (d, J = 5.4 Hz, 1H), |
| | dimethylsulfonimidoyl) | | | | 8.29 (d, J = 2.0 Hz, 1H), 7.81 |
| | pyridin-4-yl)-3-(4- | | | | (dd, J = 5.4, 2.1 Hz, 1H), 7.19 |
| | fluoro-2-methoxy-3- | | | | (dd, J = 8.8, 6.5 Hz, 1H), 6.97 |
| | methylphenyl)-4,5- | | | | (t, J = 8.9 Hz, 1H), 5.05 (d, J = |
| | dimethyl-5- | | | | 10.6 Hz, 1H), 4.28 (dd, J = |
| | (trifluoromethyl)tetrahy | | | | 10.6, 7.7 Hz, 1H), 3.72 (s, 3H), |
| | drofuran-2-carboxamide | | | | 3.14 (s, 3H), 2.73 (p, J = 7.5 |
| | (second eluting isomer | | | | Hz, 1H), 2.44 (s, 3H), 2.14 (d, |
| | by SFC on Whelk-O1 | | | | J = 2.0 Hz, 3H), 1.61 (s, 3H), |
| | column; precursor was | | | | 0.75-0.67 (m, 3H) ppm. |
| | second eluting isomer | | | | |
| | by SFC on Lux | | | | |
| | cellulose-2 column) | | | | |

### Example 5

rel-(2S,3R,4R,5S)-3-(3,4-difluorophenyl)-4,5-dimethyl-N-(2-(methylsulfonyl)pyridin-4-yl)-5-(trifluoromethyl)tetrahydrofuran-2-carboxamide (154) and rel-(2R,3S,4S,5R)-3-(3,4-difluorophenyl)-4,5-dimethyl-N-(2-(methylsulfonyl)pyridin-4-yl)-5-(trifluoromethyl)tetrahydrofuran-2-carboxamide (155)

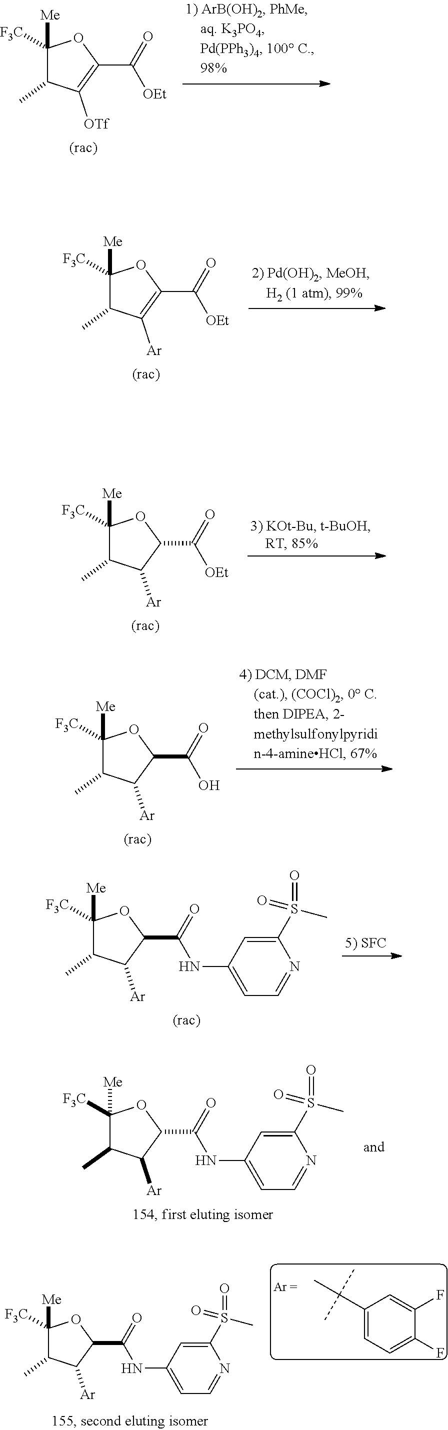

To a degassed solution of ethyl rac-(4R,5R)-4,5-dimethyl-5-(trifluoromethyl)-3-(((trifluoromethyl)sulfonyl)oxy)-4,5-dihydrofuran-2-carboxylate (2 g, 4.85 mmol) in toluene (25 mL) was added aqueous K₃PO₄ (8.5 mL of 2 M, 17.00 mmol) and (3,4-difluorophenyl)boronic acid (860 mg, 5.45 mmol). The mixture was further degassed for 10 mins before tetrakis(triphenylphosphine)palladium(0) (285 mg, 0.25 mmol) was added. The reaction was stirred at 100° C. for 2 hours before the solvent was removed in vacuo and the residue diluted with water. The aqueous layer was extracted with EtOAc (3×100 mL) and the combined organic layers were dried (MgSO₄) and concentrated in vacuo. Purification by flash chromatography (SiO₂, 2 to 5% EtOAc in hexane) gave rac-ethyl (4S,5R)-3-(3,4-difluorophenyl)-4,5-dimethyl-5-(trifluoromethyl)-4,5-dihydrofuran-2-carboxylate (1.7 g, 98%) as a colourless oil. ¹H NMR (400 MHz, DMSO-d₆) δ 7.55-7.38 (m, 2H), 7.21 (ddt, J=8.4, 4.1, 1.6 Hz, 1H), 4.20-3.98 (m, 2H), 3.78 (q, J=7.3 Hz, 1H), 1.63 (s, 3H), 1.08 (t, J=7.1 Hz, 3H), 1.02 (d, J=5.64 Hz, 3H) ppm. ESI-MS m/z calc. 350.0941, found 351.0 (M+1)⁺.

### Step 2:

Pd/C (10 wt. % loading, 456 mg, 0.43 mmol) was added to a solution of rac-ethyl (4S,5R)-3-(3,4-difluorophenyl)-4,5-dimethyl-5-(trifluoromethyl)-4,5-dihydrofuran-2-carboxylate (1.00 g, 2.86 mmol) in EtOH (50 mL) and the mixture vacuum degassed. The flask was refilled with hydrogen and a balloon of hydrogen was bubbled through the solution over 5 mins. The reaction was stirred under a balloon of hydrogen at ambient temperature for 3 hours before the balloon was refreshed and the bubbling repeated. The reaction was then left stirring under a balloon of hydrogen for 3 days. The reaction mixture was filtered through celite and the filtrate dried in vacuo to give rac-ethyl (2S,3S,4S,5R)-3-(3,4-difluorophenyl)-4,5-dimethyl-5-(trifluoromethyl)tetrahydrofuran-2-carboxylate (1000 mg, 99%) as a colourless oil which crystallised on standing. ¹H NMR (500 MHz, Chloroform-d) δ 7.16-7.10 (m, 1H), 7.09-6.95 (m, 2H), 4.83 (d, J=5.8 Hz, 1H), 4.02 (dq, J=7.1, 3.5 Hz, 2H), 3.67 (dd, J=8.5, 5.8 Hz, 1H), 2.86-2.70 (m, 1H), 1.55-1.50 (m, 3H), 0.96 (t, J=7.1 Hz, 3H), 0.86 (dq, J=7.6, 1.9 Hz, 3H) ppm.

### Step 3:

A solution of rac-ethyl (2S,3S,4S,5R)-3-(3,4-difluorophenyl)-4,5-dimethyl-5-(trifluoromethyl)tetrahydrofuran-2-carboxylate (1.26 g, 3.58 mmol) and KOt-Bu (801 mg, 7.14 mmol) in tert-butanol (34 mL) was stirred at ambient temperature for 16 hours. The reaction was diluted with EtOAc and acidified to pH 2 with 1 M HCl. The aqueous layer was further extracted with EtOAc. Te combined organic layers were dried (MgSO₄), filtered and concentrated in vacuo to give rac-(2R,3S,4S,5R)-3-(3,4-difluorophenyl)-4,5-dimethyl-5-(trifluoromethyl)tetrahydrofuran-2-carboxylic acid (1.22 g, 76%) as a pale yellow oil. ¹H NMR (500 MHz, Chloroform-d) δ 7.17 (dt, J=10.0, 8.3 Hz, 1H), 7.07 (ddd, J=11.3, 7.4, 2.3 Hz, 1H), 6.97 (ddd, J=8.5, 3.9, 1.8 Hz, 1H), 4.93 (d, J=9.6 Hz, 1H), 3.96-3.86 (m, 1H), 2.64 (p, J=7.7 Hz, 1H), 1.29 (s, 3H), 0.85 (dq, J=7.4, 2.3 Hz, 3H) ppm. ESI-MS m/z calc. 324.0785, found 323.1 (M−1)⁻.

### Step 4:

Oxalyl chloride (28 μL, 0.3210 mmol) was added to an ice-cooled solution of rac-(2R,3S,4S,5R)-3-(3,4-difluorophenyl)-4,5-dimethyl-5-(trifluoromethyl)tetrahydrofuran-2-carboxylic acid (50 mg, 0.1311 mmol) and DMF (5 μL of 0.86 M, 0.004300 mmol) in DCM (1 mL) and the mixture was warmed to ambient temperature over 30 mins before being concentrated in vacuo. The residue was dissolved in toluene (3 mL) and the mixture was concentrated in vacuo. The residue was then dissolved in DCM (1 mL) and DIPEA (51 μL, 0.2928 mmol) was added. 2-Methylsulfonylpyridin-4-amine (hydrochloride salt) (30 mg, 0.1438 mmol) was quickly added to the mixture and the reaction was stirred at RT for 1 hour. Methanol was added, and the mixture was concentrated in vacuo. The residue was purified by preparative reverse phase HPLC (basic eluent) to give rac-(2R,3S,4S,5R)-3-(3,4-difluorophenyl)-4,5-dimethyl-N-(2-(methylsulfonyl)pyridin-4-yl)-5-(trifluoromethyl)tetrahydrofuran-2-carboxamide (42 mg, 67%). ESI-MS m/z calc. 478.09857, found 479.1 (M+1)⁺; 477.0 (M−1)⁻; Retention time: 3.26 minutes.

### Step 5:

rac-(2R,3S,4S,5R)-3-(3,4-difluorophenyl)-4,5-dimethyl-N-(2-(methylsulfonyl)pyridin-4-yl)-5-(trifluoromethyl)tetrahydrofuran-2-carboxamide (42 mg, 67%) was separated by chiral SFC using a (R,R)-Whelk-01 column, 5 μm particle size, 25 cm×21.2 mm from Regis Technologies to give two single isomers of unknown absolute configuration:

First eluting isomer (rt=3.83 min): rel-(2S,3R,4R,5S)-3-(3,4-difluorophenyl)-4,5-dimethyl-N-(2-(methylsulfonyl)pyridin-4-yl)-5-(trifluoromethyl)tetrahydrofuran-2-carboxamide (154, 5 mg, 8%). ¹H NMR (500 MHz, DMSO-d₆) δ 10.83 (s, 1H), 8.64 (dd, J=5.5, 0.6 Hz, 1H), 8.40 (dd, J=2.1, 0.6 Hz, 1H), 7.92 (dd, J=5.5, 2.1 Hz, 1H), 7.48 (ddd, J=12.3, 7.8, 2.1 Hz, 1H), 7.42 (dt, J=10.7, 8.6 Hz, 1H), 7.20 (d, J=9.2 Hz, 1H), 5.15 (d, J=9.6 Hz, 1H), 4.19 (dd, J=9.6, 7.7 Hz, 1H), 3.25 (s, 3H), 2.76 (p, J=7.5 Hz, 1H), 1.62 (s, 3H), 0.84-0.66 (m, 3H) ppm. ESI-MS m/z calc. 478.09857, found 479.8 (M+1)⁺; 477.8 (M−1)⁻; Retention time: 3.24 minutes.

Second eluting isomer: (rt=7.73 min): rel-(2R,3S,4S,5R)-3-(3,4-difluorophenyl)-4,5-dimethyl-N-(2-(methylsulfonyl)pyridin-4-yl)-5-(trifluoromethyl)tetrahydrofuran-2-carboxamide (155, 4 mg, 6%). ¹H NMR (500 MHz, DMSO-d₆) δ 10.87 (br s, 1H), 8.63 (d, J=5.5 Hz, 1H), 8.40 (d, J=2.0 Hz, 1H), 7.92 (dd, J=5.5, 2.1 Hz, 1H), 7.48 (ddd, J=12.4, 7.8, 2.2 Hz, 1H), 7.42 (dt, J=10.8, 8.6 Hz, 1H), 7.29-7.10 (m, 1H), 5.15 (d, J=9.6 Hz, 1H), 4.19 (dd, J=9.6, 7.7 Hz, 1H), 3.25 (s, 3H), 2.76 (p, J=7.5 Hz, 1H), 1.62 (s, 3H), 0.78-0.72 (m, 3H) ppm. ESI-MS m/z calc. 478.09857, found 479.1 (M+1)⁺; 477.0 (M−1)⁻; Retention time: 3.23 minutes.

The following compounds were made using a method similar to that described in Example 5, except that [2-methoxy-3-(trifluoromethyl)phenyl]boronic acid was used in step 1 with Pd(dppf)Cl₂,DCM, K₂CO₃ in dioxane:water at 80° C. Methyl 5-aminopyridine-2-carboxylate was used in step 4 and General Method L was used prior to SFC as the final step. Purification by chiral SFC in the final step used Chiralpak IG column, 5 μm particle size, 25 cm×10 mm from Daicel. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 156 | rel-5-((2R,3S,4S,5R)- | 505.41 | 506.1 | 3.35 | ¹H NMR (400 MHz, |
| | 3-(2-methoxy-3- | | | | DMSO-d₆) δ 10.65 (s, 1H), |
| | (trifluoromethyl)phe | | | | 8.82 (dd, J = 2.5, 0.7 Hz, |
| | nyl)-4,5-dimethyl-5- | | | | 1H), 8.21 (dd, J = 8.6, 2.5 |
| | (trifluoromethyl)tetra | | | | Hz, 1H), 8.03-7.96 (m, |
| | hydrofuran-2- | | | | 2H), 7.72 (d, J = 7.8 Hz, |
| | carboxamido)picolin | | | | 1H), 7.67-7.61 (m, 1H), |
| | amide | | | | 7.52 (s, 1H), 7.37 (t, J = 7.8 |
| | (first eluting isomer | | | | Hz, 1H), 5.17 (d, J = 10.1 |
| | by SFC) | | | | Hz, 1H), 4.39 (dd, J = 10.2, |
| | | | | | 7.8 Hz, 1H), 3.84 (s, 3H), |
| | | | | | 2.86 (q, J = 7.5 Hz, 1H), |
| | | | | | 1.65 (s, 3H), 0.74 (d, J = |
| | | | | | 6.8 Hz, 3H) ppm. |
| 157 | rel-5-((2S,3R,4R,5S)- | 505.41 | 506.1 | 3.35 | ¹H NMR (400 MHz, |
| | 3-(2-methoxy-3- | | | | DMSO-d₆) δ 10.65 (s, 1H), |
| | (trifluoromethyl)phe | | | | 8.83 (dd, J = 2.5, 0.7 Hz, |
| | nyl)-4,5-dimethyl-5- | | | | 1H), 8.21 (dd, J = 8.6, 2.5 |
| | (trifluoromethyl)tetra | | | | Hz, 1H), 8.03-7.96 (m, |
| | hydrofuran-2- | | | | 2H), 7.75-7.69 (m, 1H), |
| | carboxamido)picolin | | | | 7.68-7.61 (m, 1H), 7.53 (s, |
| | amide | | | | 1H), 7.37 (t, J = 7.8 Hz, |
| | (second eluting | | | | 1H), 5.18 (d, J = 10.2 Hz, |
| | isomer by SFC) | | | | 1H), 4.39 (dd, J = 10.3, 7.8 |
| | | | | | Hz, 1H), 3.84 (s, 3H), 2.93- |
| | | | | | 2.80 (m, 1H), 1.65 (s, |
| | | | | | 3H), 0.74 (d, J = 7.4 Hz, |
| | | | | | 3H) ppm. |

The following compounds were made using a method similar to that described in Example 5, except an alternate Suzuki coupling reaction was used in step 1. Methyl 5-aminopyridine-2-carboxylate was used in step 4 and General Method L was used prior to SFC. Purification by chiral SFC in the final step used a Chiralpak IG column, 5 μm particle size, 25 cm×10 mm from Daicel on a Minigram SFC instrument from Berger Instruments.

### Step 1 Alternative Suzuki Reaction:

To a solution of rac-((4S,5R)-2-(ethoxycarbonyl)-4,5-dimethyl-5-(trifluoromethyl)-4,5-dihydrofuran-3-yl)boronic acid (950 mg, 3.369 mmol), 1-bromo-4-(difluoromethyl)-3-fluoro-2-methoxy-benzene (902 mg, 3.537 mmol), and Pd(dppf)Cl₂·DCM (138 mg, 0.1690 mmol) in dioxane (20 mL) was added a 2 M aqueous solution of K₃PO₄ (3.4 mL, 6.800 mmol) and the mixture was degassed and flushed with nitrogen (×3). The reaction was stirred at 100° C. for 2 hours, cooled to ambient temperature and filtered through a prepacked celite pad, washing with EtOAc and water. The layers were then separated, and the aqueous layer was extracted with EtOAc (2×5 mL). The combined organic phases were dried over MgSO₄ and filtered and concentrated in vacuo. The resulting oil was purified by flash column chromatography (SiO₂, eluting with 0 to 10% EtOAc in heptane) to give a colourless oil of rac-ethyl (4S,5R)-3-(4-(difluoromethyl)-3-fluoro-2-methoxyphenyl)-4,5-dimethyl-5-(trifluoromethyl)-4,5-dihydrofuran-2-carboxylate (915.6 mg, 57%). ¹H NMR (500 MHz, Chloroform-d) δ 7.26-7.20 (m, 1H), 7.02-6.74 (m, 2H), 4.19-4.07 (m, 2H), 3.89 (d, J=2.0 Hz, 3H), 3.51 (q, J=7.4 Hz, 1H), 1.70 (s, 3H), 1.16-1.04 (m, 3H) ppm. ESI-MS m/z calc. 412.11093, found 413.3 (M+1)⁺; Retention time: 1.06 minutes. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 158 | rel-5-((2R,3S,4S,5R)- | 505.41 | 506.3 | 3.26 | ¹H NMR (400 MHz, |
| | 3-(4- | | | | DMSO-d₆) δ 8.80 (s, 1H), |
| | (difluoromethyl)-3- | | | | 8.19 (d, J = 8.7 Hz, 1H), |
| | fluoro-2- | | | | 7.97 (s, 2H), 7.48 (s, 1H), |
| | methoxyphenyl)-4,5- | | | | 7.32 (d, J = 5.5 Hz, 2H), |
| | dimethyl-5- | | | | 7.18 (t, J = 54.2 Hz, 2H), |
| | (trifluoromethyl)tetra | | | | 5.14 (s, 1H), 4.33 (t, J = |
| | hydrofuran-2- | | | | 9.0 Hz, 1H), 3.93 (d, J = |
| | carboxamido)picolin | | | | 1.8 Hz, 3H), 2.82 (t, J = |
| | amide | | | | 7.5 Hz, 1H), 1.61 (s, 3H), |
| | (first eluting isomer | | | | 0.77-0.64 (m, 3H) ppm. |
| | by SFC) | | | | |
| 159 | rel-5-((2S,3R,4R,5S)- | 505.41 | 506.3 | 3.26 | |
| | 3-(4- | | | | |
| | (difluoromethyl)-3- | | | | |
| | fluoro-2- | | | | |
| | methoxyphenyl)-4,5- | | | | |
| | dimethyl-5- | | | | |
| | (trifluoromethyl)tetra | | | | |
| | hydrofuran-2- | | | | |
| | carboxamido)picolin | | | | |
| | amide | | | | |
| | (second eluting | | | | |
| | isomer by SFC) | | | | |

### Example 6

rel-(2S,3R,4R,5S)-N-([1,2,3]triazolo[1,5-a]pyridin-6-yl)-3-(3-(difluoromethyl)-4-fluoro-2-methoxyphenyl)-4,5-dimethyl-5-(trifluoromethyl)tetrahydrofuran-2-carboxamide (160) and rel-(2R,3S,4S,5R)—N-([1,2,3]triazolo[1,5-a]pyridin-6-yl)-3-(3-(difluoromethyl)-4-fluoro-2-methoxyphenyl)-4,5-dimethyl-5-(trifluoromethyl)tetrahydrofuran-2-carboxamide (161)

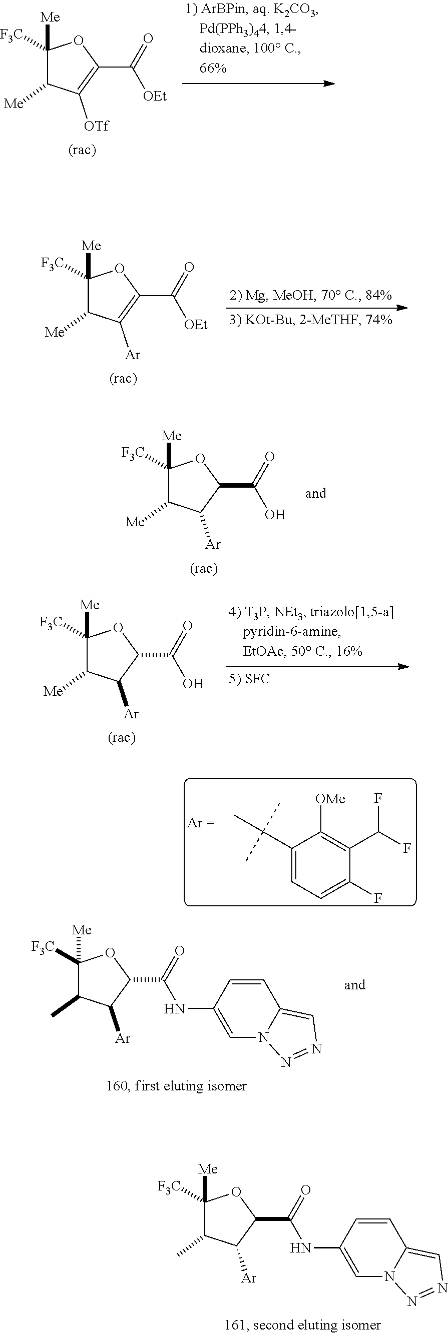

### Step 1:

A mixture of ethyl rac-(4R,5R)-4,5-dimethyl-5-(trifluoromethyl)-3-(((trifluoromethyl)sulfonyl)oxy)-4,5-dihydrofuran-2-carboxylate (1.44 g, 3.169 mmol), 2-(3-(difluoromethyl)-4-fluoro-2-methoxyphenyl)-4,4,5,5-tetramethyl-1,3,2-dioxaborolane (900 mg, 2.592 mmol), Pd(Ph₃)₄(148 mg, 0.1281 mmol), and aqueous K₂CO₃ (2.6 mL of 2 M, 5.200 mmol) in 1,4-dioxane (25 mL) was heated at 100° C. for 2 h. The mixture was concentrated in vacuo and loaded onto solid support. Purification by flash chromatography (SiO₂, 0 to 25% EtOAc in heptane) gave ethyl rac-(4S,5R)-3-(3-(difluoromethyl)-4-fluoro-2-methoxyphenyl)-4,5-dimethyl-5-(trifluoromethyl)-4,5-dihydrofuran-2-carboxylate (708 mg, 66%) as a colourless oil. ¹H NMR (400 MHz, Chloroform-d) δ 7.25 (ddt, J=7.3, 6.2, 1.2 Hz, 1H), 6.95 (td, J=53.6, 0.7 Hz, 1H), 6.94 (tt, J=8.7, 0.9 Hz, 1H), 4.17 (qd, J=7.1, 1.3 Hz, 2H), 3.77 (s, 3H), 3.62-3.53 (m, 1H), 1.71 (q, J=1.0 Hz, 3H), 1.15 (t, J=7.1 Hz, 3H), 1.07 (dq, J=7.1, 2.2 Hz, 3H) ppm. ESI-MS m/z calc. 412.11093, found 413.2 (M+1)⁺; Retention time: 1.05 minutes.

### Step 2:

A solution of ethyl rac-(4S,5R)-3-(3-(difluoromethyl)-4-fluoro-2-methoxyphenyl)-4,5-dimethyl-5-(trifluoromethyl)-4,5-dihydrofuran-2-carboxylate (3.5 g, 8.488 mmol) in MeOH (100 mL) was added to a two necked flask containing magnesium (2.07 g, 85.17 mmol). The reaction mixture was heated at 70° C. for 3 h. The mixture was concentrated in vacuo and partitioned between aqueous AcOH and EtOAc. The aqueous layer was separated and extracted twice with EtOAc. The combined organic phases were washed with aqueous NaHCO₃ and twice with water. The organic phase was dried (MgSO₄) and concentrated in vacuo to give methyl rac-(2S,3S,4S,5R)-3-(3-(difluoromethyl)-4-fluoro-2-methoxyphenyl)-4,5-dimethyl-5-(trifluoromethyl)tetrahydrofuran-2-carboxylate and methyl rac-(2R,3R,4S,5R)-3-(3-(difluoromethyl)-4-fluoro-2-methoxyphenyl)-4,5-dimethyl-5-(trifluoromethyl)tetrahydrofuran-2-carboxylate (2.87 g, 84%) as an orange oil. ¹H NMR (400 MHz, Chloroform-d) δ 7.43-7.32 (m, 1H), 7.01-6.95 (m, 1H), 7.09-6.80 (m, 1H), 4.89 (d, J=10.2 Hz, 1H), 4.21-4.15 (m, 1H), 3.84 (s, 3H), 3.71 (s, 3H), 2.73 (p, J=7.7 Hz, 1H), 1.63 (q, J=1.2 Hz, 3H), 0.78 (ddq, J=7.2, 4.7, 2.3 Hz, 3H) ppm.

### Step 3:

Potassium tert-butoxide (1.66 g, 14.79 mmol) was added to a solution of methyl rac-(2S,3S,4S,5R)-3-(3-(difluoromethyl)-4-fluoro-2-methoxyphenyl)-4,5-dimethyl-5-(trifluoromethyl)tetrahydrofuran-2-carboxylate and methyl rac-(2R,3R,4S,5R)-3-(3-(difluoromethyl)-4-fluoro-2-methoxyphenyl)-4,5-dimethyl-5-(trifluoromethyl)tetrahydrofuran-2-carboxylate (2.87 g, 7.169 mmol) in 2-MeTHF (35 mL) in a water bath at ambient temperature. During addition, a -3° exotherm was observed. The reaction mixture was stirred for 2 hours, after which time a further portion of potassium tert-butoxide (860 mg) was added. The mixture was stirred at ambient temperature for a further 1 hour before quenching with an aqueous HCl solution. The aqueous layer was separated and washed with EtOAc, dried (MgSO₄) and concentrated in vacuo to give rac-(2R,3S,4S,5R)-3-(3-(difluoromethyl)-4-fluoro-2-methoxyphenyl)-4,5-dimethyl-5-(trifluoromethyl)tetrahydrofuran-2-carboxylic acid and rac-(2S,3R,4S,5R)-3-(3-(difluoromethyl)-4-fluoro-2-methoxyphenyl)-4,5-dimethyl-5-(trifluoromethyl)tetrahydrofuran-2-carboxylic acid (3.32 g, 74%) in a ratio -3:2 (no assignment) and as an orange oil. ¹H NMR (400 MHz, Chloroform-d) δ 7.43-7.35 (m, 1H), 6.98 (ddd, J=13.4, 9.3, 4.2 Hz, 1H), 6.93 (t, J=53.6 Hz, 1H), 4.93 (d, J=10.3 Hz, 1H), 4.18-4.14 (m, 1H), 3.84 (s, 3H), 2.76 (p, J=7.7 Hz, 1H), 1.67-1.62 (m, 3H), 0.82-0.75 (m, 3H) ppm; OH acid not observed. ESI-MS m/z calc. 386.09528, found 385.1 (M−1)⁻; Retention time: 0.57 minutes.

Major diastereomer: rac-(2R,3S,4S,5R)-3-(3-(difluoromethyl)-4-fluoro-2-methoxyphenyl)-4,5-dimethyl-5-(trifluoromethyl)tetrahydrofuran-2-carboxylic acid ¹H NMR (400 MHz, Chloroform-d) δ 7.43-7.35 (m, 1H), 7.04-6.91 (m, 1H), 6.93-6.72 (m, 1H), 4.93 (d, J=10.3 Hz, 1H), 4.18-4.14 (m, 1H), 3.84 (s, 3H), 2.76 (m, 1H), 1.27 (m, 3H), 1.03 (m, 3H); OH acid not observed.

Minor diastereomer: rac-(2S,3R,4S,5R)-3-(3-(difluoromethyl)-4-fluoro-2-methoxyphenyl)-4,5-dimethyl-5-(trifluoromethyl)tetrahydrofuran-2-carboxylic acid. ¹H NMR (400 MHz, Chloroform-d) δ 7.43-7.35 (m, 1H), 7.04-6.91 (m, 1H), 6.93-6.72 (m, 1H), 4.62 (d, J=9.9 Hz, 1H), 3.97 (m, 2H), 3.81 (s, 3H), 2.26 (m, 1H), 1.23 (m, 3H), 0.78 (m, 3H); OH acid not observed.

### Step 4:

To a solution containing a 3:2 mixture of rac-(2R,3S,4S,5R)-3-(3-(difluoromethyl)-4-fluoro-2-methoxyphenyl)-4,5-dimethyl-5-(trifluoromethyl)tetrahydrofuran-2-carboxylic acid and rac-(2S,3R,4S,5R)-3-(3-(difluoromethyl)-4-fluoro-2-methoxyphenyl)-4,5-dimethyl-5-(trifluoromethyl)tetrahydrofuran-2-carboxylic acid (150 mg, 0.3883 mmol), triazolo[1,5-a]pyridin-6-amine (66.3 mg, 0.4943 mmol) and TEA (165 μL, 1.184 mmol) in ethyl acetate (3 mL) was added T3P (360 μL of 50% w/w, 0.6048 mmol) and the reaction was stirred overnight at ambient temperature. The reaction was partitioned between ethyl acetate and water. The aqueous layer was extracted twice with ethyl acetate and the combined organics were dried with MgSO₄ and concentrated in vacuo. The crude product was purified by preparative reverse phase HPLC (basic eluent) to give rac-(2R,3S,4S,5R)—N-([1,2,3]triazolo[1,5-a]pyridin-6-yl)-3-(3-(difluoromethyl)-4-fluoro-2-methoxyphenyl)-4,5-dimethyl-5-(trifluoromethyl)tetrahydrofuran-2-carboxamide (31 mg, 16%). ¹H NMR (500 MHz, Chloroform-d) δ 9.60 (p, J=1.0 Hz, 1H), 8.37 (s, 1H), 8.03 (d, J=1.0 Hz, 1H), 7.69 (dd, J=9.4, 1.0 Hz, 1H), 7.56-7.49 (m, 1H), 7.13 (dd, J=9.4, 1.7 Hz, 1H), 7.07-6.82 (m, 2H), 5.04 (d, J=10.6 Hz, 1H), 4.16 (dd, J=10.6, 8.4 Hz, 1H), 3.86 (s, 3H), 2.80 (p, J=7.9 Hz, 1H), 1.70 (s, 3H), 0.81 (dt, J=7.6, 2.5 Hz, 3H) ppm. ESI-MS m/z calc. 502.14395, found 503.4 (M+1)⁺; 501.4 (M−1)⁻; Retention time: 3.31 minutes.

### Step 5:

rac-(2R,3S,4S,5R)—N-([1,2,3]triazolo[1,5-a]pyridin-6-yl)-3-(3-(difluoromethyl)-4-fluoro-2-methoxyphenyl)-4,5-dimethyl-5-(trifluoromethyl)tetrahydrofuran-2-carboxamide (30 mg, 0.05971 mmol) was purified by chiral SFC using a Lux Cellulose-2 column, 5 μm particle size, 25 cm×10 mm from Phenomenex, Inc. on a Minigram SFC instrument from Berger Instruments to give:

First eluting isomer (rt=2.61 minutes): rel-(2S,3R,4R,5S)-N-([1,2,3]triazolo[1,5-a]pyridin-6-yl)-3-(3-(difluoromethyl)-4-fluoro-2-methoxyphenyl)-4,5-dimethyl-5-(trifluoromethyl)tetrahydrofuran-2-carboxamide (160, 11 mg, 73%). ¹H NMR (500 MHz, DMSO-d₆) δ 10.59 (s, 1H), 9.54 (s, 1H), 8.14 (s, 1H), 7.92 (d, J=9.5 Hz, 1H), 7.63 (dd, J=8.8, 6.2 Hz, 1H), 7.43 (dd, J=9.5, 1.7 Hz, 1H), 7.32-7.07 (m, 2H), 5.14 (d, J=10.2 Hz, 1H), 4.31 (dd, J=10.3, 7.7 Hz, 1H), 3.83 (s, 3H), 2.79 (p, J=7.6 Hz, 1H), 1.64 (s, 3H), 0.75 (d, J=7.4 Hz, 3H) ppm. ESI-MS m/z calc. 502.14395, found 503.2 (M+1)⁺; 501.2 (M−1)⁻; Retention time: 3.31 minutes.

Second eluting isomer (rt=3.41 minutes): rel-(2R,3S,4S,5R)—N-([1,2,3]triazolo[1,5-a]pyridin-6-yl)-3-(3-(difluoromethyl)-4-fluoro-2-methoxyphenyl)-4,5-dimethyl-5-(trifluoromethyl)tetrahydrofuran-2-carboxamide (161, 10 mg, 64%). ¹H NMR (500 MHz, DMSO-d₆) δ 10.58 (s, 1H), 9.54 (s, 1H), 8.14 (s, 1H), 7.93 (d, J=9.5 Hz, 1H), 7.63 (dd, J=8.8, 6.2 Hz, 1H), 7.44 (dd, J=9.5, 1.7 Hz, 1H), 7.32-7.05 (m, 2H), 5.15 (d, J=10.2 Hz, 1H), 4.31 (dd, J=10.2, 7.8 Hz, 1H), 3.83 (s, 3H), 2.79 (p, J=7.6 Hz, 1H), 1.64 (s, 3H), 0.77-0.73 (m, 3H) ppm. ESI-MS m/z calc. 502.14395, found 503.2 (M+1)⁺; 501.3 (M−1)⁻; Retention time: 3.31 minutes.

The following compounds were made using a method similar to that described in Example 6, except that different amines were used in step 4. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 162 | rel-(2R,3S,4S,5R)-3- | 574.515 | 575.7 | 3.24 | ¹H NMR (500 MHz, |
| | (3-(difluoromethyl)- | | | | Methanol-d₄) δ 8.31 (d, J = |
| | 4-fluoro-2- | | | | 5.7 Hz, 1H), 7.96 (d, J = 1.9 |
| | methoxyphenyl)-4,5- | | | | Hz, 1H), 7.60-7.54 (m, 2H), |
| | dimethyl-N-(2-(4- | | | | 7.17-6.88 (m, 2H), 5.06 (d, |
| | methyl-2- | | | | J = 10.3 Hz, 1H), 4.35 (dd, J = |
| | oxopiperazin-1- | | | | 10.3, 8.2 Hz, 1H), 3.94- |
| | yl)pyridin-4-yl)-5- | | | | 3.88 (m, 2H), 3.86 (s, 3H), |
| | (trifluoromethyl)tetra | | | | 3.27 (s, 2H), 2.84 (dd, J = |
| | hydrofuran-2- | | | | 6.4, 4.8 Hz, 2H), 2.80 (d, J = |
| | carboxamide | | | | 7.7 Hz, 1H), 2.40 (s, 3H), |
| | (first eluting isomer | | | | 1.67 (d, J = 1.4 Hz, 3H), 0.82 |
| | by SFC using a | | | | (dt, J = 7.4, 2.4 Hz, 3H) ppm. |
| | Chiralpak IB | | | | |
| | column) | | | | |
| 163 | rel-(2S,3R,4R,5S)-3- | 574.515 | 575.7 | 3.24 | ¹H NMR (500 MHz, |
| | (3-(difluoromethyl)- | | | | Methanol-d₄) δ 8.31 (d, J = |
| | 4-fluoro-2- | | | | 5.7 Hz, 1H), 7.96 (d, J = 1.8 |
| | methoxyphenyl)-4,5- | | | | Hz, 1H), 7.59 (dd, J = 5.7, |
| | dimethyl-N-(2-(4- | | | | 2.0 Hz, 1H), 7.57-7.54 (m, |
| | methyl-2- | | | | 1H), 7.17-6.86 (m, 2H), |
| | oxopiperazin-1- | | | | 5.06 (d, J = 10.4 Hz, 1H), |
| | yl)pyridin-4-yl)-5- | | | | 4.35 (dd, J = 10.4, 8.2 Hz, |
| | (trifluoromethyl)tetra | | | | 1H), 3.94-3.88 (m, 2H), |
| | hydrofuran-2- | | | | 3.85 (s, 3H), 3.27 (s, 2H), |
| | carboxamide | | | | 2.84 (dd, J = 6.2, 4.9 Hz, |
| | (second eluting | | | | 2H), 2.80 (q, J = 7.7 Hz, 1H), |
| | isomer by SFC using | | | | 2.40 (s, 3H), 1.68-1.65 (m, |
| | a Chiralpak IB | | | | 3H), 0.82 (dq, J = 7.4, 2.4 |
| | column) | | | | Hz, 3H) ppm. |
| 164 | rel-(2S,3R,4R,5S)-3- | 512.393 | 513.6 | 3.6 | ¹H NMR (500 MHz, |
| | (3-(difluoromethyl)- | | | | Methanol-d₄) δ 8.83 (d, J = |
| | 4-fluoro-2- | | | | 2.4 Hz, 1H), 8.26 (dd, J = |
| | methoxyphenyl)-N- | | | | 8.5, 2.5 Hz, 1H), 7.65 (d, J = |
| | (6- | | | | 8.5 Hz, 1H), 7.59 (dd, J = |
| | (difluoromethyl)pyri | | | | 8.9, 6.0 Hz, 1H), 7.18-6.90 |
| | din-3-yl)-4,5- | | | | (m, 2H), 6.66 (t, J = 55.3 Hz, |
| | dimethyl-5- | | | | 1H), 5.10 (d, J = 10.3 Hz, |
| | (trifluoromethyl)tetra | | | | 1H), 4.36 (dd, J = 10.4, 8.2 |
| | hydrofuran-2- | | | | Hz, 1H), 3.86 (s, 3H), 2.83 |
| | carboxamide | | | | (p, J = 7.7 Hz, 1H), 1.69 (d, J = |
| | (first eluting isomer | | | | 1.2 Hz, 3H), 0.83 (dt, J = |
| | by SFC using a | | | | 7.6, 2.3 Hz, 3H) ppm. |
| | Whelk-O1 column) | | | | |
| 165 | rel-(2R,3S,4S,5R)-3- | 512.393 | 513.6 | 3.6 | ¹H NMR (500 MHz, |
| | (3-(difluoromethyl)- | | | | Methanol-d₄) δ 8.83 (d, J = |
| | 4-fluoro-2- | | | | 2.4 Hz, 1H), 8.26 (dd, J = |
| | methoxyphenyl)-N- | | | | 8.5, 2.5 Hz, 1H), 7.64 (d, J = |
| | (6- | | | | 8.6 Hz, 1H), 7.59 (dd, J = |
| | (difluoromethyl)pyri | | | | 8.9, 6.1 Hz, 1H), 7.18-6.87 |
| | din-3-yl)-4,5- | | | | (m, 2H), 6.66 (t, J = 55.3 Hz, |
| | dimethyl-5- | | | | 1H), 5.10 (d, J = 10.4 Hz, |
| | (trifluoromethyl)tetra | | | | 1H), 4.36 (dd, J = 10.4, 8.2 |
| | hydrofuran-2- | | | | Hz, 1H), 3.86 (s, 3H), 2.82 |
| | carboxamide | | | | (p, J = 7.7 Hz, 1H), 1.68 (d, J = |
| | (second eluting | | | | 1.2 Hz, 3H), 0.83 (dq, J = |
| | isomer by SFC using | | | | 7.3, 2.3 Hz, 3H) ppm. |
| | a Whelk-O1 column) | | | | |
| 166 | rel-(2S,3R,4R,5S)-3- | 492.411 | 493.6 | 3.53 | ¹H NMR (500 MHz, |
| | (3-(difluoromethyl)- | | | | Methanol-d₄) δ 8.28 (dd, J = |
| | 4-fluoro-2- | | | | 2.7, 0.7 Hz, 1H), 7.59 (dd, J = |
| | methoxyphenyl)-N- | | | | 8.9, 6.1 Hz, 1H), 7.22- |
| | (6-methoxypyridin- | | | | 6.89 (m, 2H), 6.76 (dd, J = |
| | 3-yl)-4,5-dimethyl-5- | | | | 8.9, 0.7 Hz, 1H), 5.05 (d, J = |
| | (trifluoromethyl)tetra | | | | 10.5 Hz, 1H), 4.32 (dd, J = |
| | hydrofuran-2- | | | | 10.5, 8.2 Hz, 1H), 3.87 (s, |
| | carboxamide | | | | 3H), 3.85 (s, 3H), 2.81 (p, J = |
| | (first eluting isomer | | | | 7.7 Hz, 1H), 1.68 (d, J = 1.4 |
| | by SFC using a Lux | | | | Hz, 3H), 0.82 (dq, J = 7.4, |
| | Cellulose-2 column) | | | | 2.3 Hz, 3H) ppm. |
| 167 | rel-(2R,3S,4S,5R)-3- | 492.411 | 493.6 | 3.53 | ¹H NMR (500 MHz, |
| | (3-(difluoromethyl)- | | | | Methanol-d₄) δ 8.28 (dd, J = |
| | 4-fluoro-2- | | | | 2.7, 0.7 Hz, 1H), 7.84 (dd, J = |
| | methoxyphenyl)-N- | | | | 8.9, 2.7 Hz, 1H), 7.59 (dd, |
| | (6-methoxypyridin- | | | | J = 8.9, 6.0 Hz, 1H), 7.19- |
| | 3-yl)-4,5-dimethyl-5- | | | | 6.88 (m, 2H), 6.76 (dd, J = |
| | (trifluoromethyl)tetra | | | | 9.0, 0.7 Hz, 1H), 5.05 (d, J = |
| | hydrofuran-2- | | | | 10.4 Hz, 1H), 4.32 (dd, J = |
| | carboxamide | | | | 10.5, 8.2 Hz, 1H), 3.87 (s, |
| | (second eluting | | | | 3H), 3.85 (s, 3H), 2.81 (p, J = |
| | isomer by SFC using | | | | 7.7 Hz, 1H), 1.68 (d, J = 1.2 |
| | a Lux Cellulose-2 | | | | Hz, 3H), 0.82 (dq, J = 7.4, |
| | column) | | | | 2.3 Hz, 3H) ppm. |

The following compounds were made using a method similar to that described in Example 6, except that methyl 5-aminopyridine -2-carboxylate was used in step 4. General Method L was used on the product of step 4 (using NH₃ or NHMe). Purification by chiral SFC was carried out as the final step using a Chiralpak IG column, 5 μm particle size, 25 cm×10 mm from Daicel.

| Cmpd | | LC/MS | Found | MS | |
|---|---|---|---|---|---|
| No. | Compound Name | (m/z calc.) | M + 1 | r.t. | NMR (shifts in ppm) |
| 168 | rel-5-((2R,3S,4S,5R)- | 505.41 | 506.25 | 3.28 | ¹H NMR (500 MHz, |
| | 3-(3- | | | | Methanol-d₄) δ 8.85 (d, J = |
| | (difluoromethyl)-4- | | | | 2.5 Hz, 1H), 8.20 (dd, J = |
| | fluoro-2- | | | | 8.6, 2.5 Hz, 1H), 8.05 (d, J = |
| | methoxyphenyl)-4,5- | | | | 8.6 Hz, 1H), 7.59 (dd, J = |
| | dimethyl-5- | | | | 8.8, 6.0 Hz, 1H), 7.19- |
| | (trifluoromethyl)tetra | | | | 6.88 (m, 2H), 5.10 (d, J = |
| | hydrofuran-2- | | | | 10.4 Hz, 1H), 4.36 (dd, J = |
| | carboxamido)picolin | | | | 10.3, 8.2 Hz, 1H), 3.86 (s, |
| | amide | | | | 3H), 2.82 (p, J = 7.7 Hz, |
| | (first eluting isomer | | | | 1H), 1.68 (s, 3H), 0.85- |
| | by SFC) | | | | 0.80 (m, 3H) ppm. |
| | | | | | ¹H NMR (500 MHz, |
| | | | | | Chloroform-d) δ 8.72 (dd, J = |
| | | | | | 2.1, 1.1 Hz, 1H), 8.52 (s, |
| | | | | | 1H), 8.20 (t, J = 1.5 Hz, |
| | | | | | 2H), 7.72 (s, 1H), 7.57- |
| | | | | | 7.51 (m, 1H), 7.07-6.82 |
| | | | | | (m, 2H), 5.51 (s, 1H), 5.05 |
| | | | | | (d, J = 10.7 Hz, 1H), 4.17 |
| | | | | | (dd, J = 10.7, 8.3 Hz, 1H), |
| | | | | | 3.87 (s, 3H), 2.82 (p, J = |
| | | | | | 7.7 Hz, 1H), 1.72 (d, J = 1.2 |
| | | | | | Hz, 3H), 0.84 (dd, J = 7.8, |
| | | | | | 2.3 Hz, 3H) ppm. |
| 169 | rel-5-((2S,3R,4R,5S)- | 505.41 | 506.25 | 3.28 | ¹H NMR (500 MHz, |
| | 3-(3- | | | | Chloroform-d) δ 8.72 (dd, J = |
| | (difluoromethyl)-4- | | | | 2.1, 1.2 Hz, 1H), 8.53 (s, |
| | fluoro-2- | | | | 1H), 8.20 (t, J = 1.4 Hz, |
| | methoxyphenyl)-4,5- | | | | 2H), 7.72 (s, 1H), 7.60- |
| | dimethyl-5- | | | | 7.50 (m, 1H), 7.09-6.79 |
| | (trifluoromethyl)tetra | | | | (m, 2H), 5.43 (d, J = 102.9 |
| | hydrofuran-2- | | | | Hz, 1H), 5.05 (d, J = 10.7 |
| | carboxamido)picolin | | | | Hz, 1H), 4.17 (dd, J = 10.7, |
| | amide | | | | 8.4 Hz, 1H), 3.87 (s, 3H), |
| | (second eluting | | | | 2.82 (p, J = 7.7 Hz, 1H), |
| | isomer by SFC) | | | | 1.75-1.70 (m, 3H), 0.83 |
| | | | | | (dt, J = 7.5, 2.3 Hz, 3H) |
| | | | | | ppm. |
| 170 | rel-5-((2R,3S,4S,5R)- | 519.437 | 520.4 | 3.3 | ¹H NMR (500 MHz, |
| | 3-(3- | | | | Methanol-d₄) δ 8.86 (dd, J = |
| | (difluoromethyl)-4- | | | | 2.5, 0.7 Hz, 1H), 8.19 |
| | fluoro-2- | | | | (dd, J = 8.6, 2.5 Hz, 1H), |
| | methoxyphenyl)-4,5- | | | | 8.03 (dd, J = 8.6, 0.7 Hz, |
| | dimethyl-5- | | | | 1H), 7.60 (dd, J = 8.9, 6.0 |
| | (trifluoromethyl)tetra | | | | Hz, 1H), 7.23-6.91 (m, |
| | hydrofuran-2- | | | | 2H), 5.11 (d, J = 10.3 Hz, |
| | carboxamido)-N- | | | | 1H), 4.37 (dd, J = 10.4, 8.2 |
| | methylpicolinamide | | | | Hz, 1H), 3.87 (s, 3H), 2.95 |
| | (TFA salt) | | | | (s, 3H), 2.83 (p, J = 7.7 Hz, |
| | (first eluting isomer | | | | 1H), 1.69 (d, J = 1.2 Hz, |
| | by SFC) | | | | 3H), 0.84 (dt, J = 7.4, 2.4 |
| | | | | | Hz, 3H) ppm. |
| 171 | rel-5-((2S,3R,4R,5S)- | 519.437 | 520.4 | 3.31 | ¹H NMR (500 MHz, |
| | 3-(3- | | | | Methanol-d₄) δ 8.85 (dd, J = |
| | (difluoromethyl)-4- | | | | 2.5, 0.7 Hz, 1H), 8.18 |
| | fluoro-2- | | | | (dd, J = 8.6, 2.5 Hz, 1H), |
| | methoxyphenyl)-4,5- | | | | 8.02 (dd, J = 8.5, 0.7 Hz, |
| | dimethyl-5- | | | | 1H), 7.59 (dd, J = 8.9, 6.0 |
| | (trifluoromethyl)tetra | | | | Hz, 1H), 7.18-6.87 (m, |
| | hydrofuran-2- | | | | 2H), 5.10 (d, J = 10.4 Hz, |
| | carboxamido)-N- | | | | 1H), 4.36 (dd, J = 10.4, 8.2 |
| | methylpicolinamide | | | | Hz, 1H), 3.86 (s, 3H), 2.94 |
| | (TFA salt) | | | | (s, 3H), 2.82 (p, J = 7.7 Hz, |
| | (second eluting | | | | 1H), 1.74-1.65 (m, 3H), |
| | isomer by SFC) | | | | 0.83 (dq, J = 7.3, 2.3 Hz, |
| | | | | | 3H) ppm. |

The following compounds were made using a method similar to that described in Example 6, except that methyl 5-aminopyrimidine-2-carboxylate was used in step 4. General Method L was used prior to SFC separation as the final step. Purification by chiral SFC in the final step used a Lux Cellulose-2 column, 5 μm particle size, 25 cm×10 mm from Phenomenex, Inc. on a Minigram SFC instrument from Berger Instruments. In the Table below, “MS r.t.” stands for Mass Spec retention time.

| | | LC/MS | Found | MS | |
|---|---|---|---|---|---|
| Cmpd. No. | Compound Name | (m/z calc.) | M + 1 | r.t. | NMR (shifts in ppm) |
| 172 | rel-5((2S,3R,4R,5S)- | 506.398 | 507.4 | 3.04 | ¹H NMR (500 MHz, |
| | 3-(3-(difluoromethyl)- | | | | DMSO-d₆) δ 10.66 (s, 1H), |
| | 4-fluoro-2- | | | | 9.14 (s, 2H), 8.10 (s, 1H), |
| | methoxy-phenyl)-4,5- | | | | 7.68 (s, 1H), 7.65 (dd, J = |
| | dimethyl-5- | | | | 8.9, 6.3 Hz, 1H), 7.34-7.09 |
| | (trifluoromethyl) | | | | (m, 2H), 5.18 (d, J = 10.2 |
| | tetrahydrofuran-2- | | | | Hz, 1H), 4.29 (dd, J = 10.2, |
| | carboxamido) | | | | 7.9 Hz, 1H), 3.81 (s, 3H), |
| | pyrimidine-2-carboxamide | | | | 2.79 (p, J = 7.5 Hz, 1H), |
| | (TFA salt) | | | | 1.65 (s, 3H), 0.74 (d, J = |
| | (first eluting isomer | | | | 7.5 Hz, 3H) ppm. |
| | by SFC) | | | | |
| 173 | rel-5-((2R,3S,4S,5R)- | 506.398 | 507.4 | 3.04 | ¹H NMR (500 MHz, |
| | 3-(3-(difluoromethyl)- | | | | DMSO-d₆) δ 10.67 (s, 1H), |
| | 4-fluoro-2- | | | | 9.14 (s, 2H), 8.10 (s, 1H), |
| | methoxyphenyl)-4,5- | | | | 7.68 (s, 1H), 7.65 (dd, J = |
| | dimethyl-5- | | | | 8.9, 6.3 Hz, 1H), 7.32-7.09 |
| | (trifluoromethyl) | | | | (m, 2H), 5.18 (d, J = 10.2, |
| | tetrahydrofuran-2- | | | | 7.9 Hz, 1H), 3.81 (s, 3H), |
| | carboxamido)pyrimidine- | | | | 2.79 (p, J = 7.6 Hz, 1H), |
| | 2-carboxamide | | | | 1.65 (s, 3H), 0.74 (d, J = |
| | (TFA salt) | | | | 7.4 Hz, 3H) ppm. |
| | (second eluting isomer | | | | |
| | by SFC) | | | | |

The following compounds were made using a method similar to that described in Example 6, except that different amines were used in step 4. General Method 0 was used on the separated isomers from step 4 SFC as the final step. In the Table below, “MS r.t.” stands for Mass Spec retention time.

| | | LC/MS | Found | MS | |
|---|---|---|---|---|---|
| Cmpd. No. | Compound Name | (m/z calc.) | M + 1 | r.t. | NMR (shifts in ppm) |
| 174 | rel-4-((2R,3S,4S,5R)- | 506.395 | 507.4 | 2.49 | ¹H NMR (500 MHz, |
| | 3-(3- | | | | DMSO-d₆) δ 10.73 (d, J = |
| | difluoromethyl)-4- | | | | 4.7 Hz, 1H), 8.56 (d, J = 5.6 |
| | fluoro-2- | | | | Hz, 1H), 8.35 (s, 1H), 7.88 |
| | methoxyphenyl)-4,5- | | | | (d, J = 5.3 Hz, 1H), 7.66-7.53 |
| | dimethyl-5- | | | | (m, 1H), 7.40-6.92 (m, 2H), |
| | (trifluoromethyl) | | | | 5.13 (d, J = 10.1 Hz, 1H), 4.28 |
| | tetrahydrofuran-2- | | | | (dd, J = 10.2, 7.9 Hz, 1H), |
| | carboxamido) | | | | 3.81 (s, 3H), 2.79 (t, J = 7.5 |
| | picolinic acid | | | | Hz, 1H), 1.63 (s, 3H), 0.74 |
| | (precursor was first | | | | (d, J = 7.5 Hz, 3H) ppm. |
| | eluting isomer by | | | | |
| | SFC using Lux | | | | |
| | i-Cellulose-5 column) | | | | |
| 175 | rel-4-((2S,3R,4R,5S)- | 506.395 | 507.4 | 2.49 | ¹H NMR (500 MHz, |
| | 3-(3- | | | | DMSO-d₆) δ 10.71 (d, J = |
| | (difluoromethyl)-4- | | | | 10.4 Hz, 1H), 8.56 (dd, J = |
| | fluoro-2- | | | | 5.5, 1.9 Hz, 1H), 8.35 (s, |
| | methoxyphenyl)-4,5- | | | | 1H), 7.86 (s, 1H), 7.62 (t, J = |
| | dimethyl-5- | | | | 7.7 Hz, 1H), 7.38-6.92 |
| | (trifluoromethyl) | | | | (m, 2H), 5.22-5.05 (m, 1H), |
| | tetrahydrofuran-2- | | | | 4.36-4.17 (m, 1H), 3.81 (s, 3H), |
| | carboxamido) | | | | 2.79 (t, J = 7.6 Hz, 1H), 1.63 |
| | picolinic acid | | | | (s, 3H), 0.74 (d, J = 7.4 Hz, |
| | (precursor was | | | | 3H) ppm. |
| | second eluting | | | | |
| | isomer by SFC using | | | | |
| | Lux i-Cellulose-5 | | | | |
| | column) | | | | |
| 176 | rel-5-((2R,3S,4S,5R)- | 506.395 | 507.6 | 2.56 | ¹H NMR (500 MHz, |
| | 3-(3- | | | | Methanol-d₄) δ 8.83 (s, 1H), |
| | (difluoromethyl)-4- | | | | 8.23 (dd, J = 8.6, 2.5 Hz, 1H), |
| | fluoro-2- | | | | 8.06 (d, J = 8.6 Hz, 1H), 7.59 |
| | methoxyphenyl)-4,5- | | | | (dd, J = 8.9, 6.1 Hz, 1H), |
| | dimethyl-5- | | | | 7.21-6.88 (m, 2H), 5.10 (d, J = |
| | (trifluoromethyl) | | | | 10.3 Hz, 1H), 4.36 (dd, J = 10.4, |
| | tetrahydrofuran-2- | | | | 8.2 Hz, 1H), 3.86 (s, 3H), 2.82 |
| | carboxamido) | | | | (p, J = 7.7 Hz, 1H), 1.68 (s, |
| | picolinic acid | | | | 3H), 0.83 (dq, J = 7.5, 2.3 Hz, |
| | (precursor was first | | | | 3H) ppm. |
| | eluting isomer by | | | | |
| | SFC using Chiralpak | | | | |
| | IG column) | | | | |
| 177 | rel-5-((2S,3R,4R,5S)- | 506.395 | 507.6 | 2.56 | ¹H NMR (500 MHz, |
| | 3-(3- | | | | Methanol-d₄) δ 8.84 (s, J = |
| | (difluoromethyl)-4- | | | | 2.5 Hz, 1H), 8.24 (dd, J = 8.6, |
| | fluoro-2- | | | | 2.5 Hz, 1H), 8.07 (d, J = 8.6 |
| | methoxyphenyl)-4,5- | | | | Hz, 1H), 7.59 (dd, J = 8.8, 6.0 |
| | dimethyl-5- | | | | Hz, 1H), 7.22-6.90 (m, 2H), 5.10 |
| | (trifluoromethyl) | | | | (d, J = 10.4 Hz, 1H), 4.36 (dd, |
| | tetrahydrofuran-2- | | | | J = 10.4, 8.2 Hz, 1H), 3.86 (s, |
| | carboxamido) | | | | 3H), 2.82 (p, J = 7.7 Hz, 1H), |
| | picolinic acid | | | | 1.68 (s, 3H), 0.83 (dq, J = 7.7, |
| | (precursor was | | | | 2.3 Hz, 3H) ppm. |
| | second eluting isomer | | | | |
| | by SFC using | | | | |
| | Chiralpak IG column) | | | | |

The following compounds were made using a method similar to that described in Example 6, except in step 1 ((4S,5R)-2-(ethoxycarbonyl)-4,S-dimethyl-5-(trifluoromethyl)-4,S-dihydrofuran-3-yl)boronic acid and 1-bromo-4-fluoro-2-methoxy-3-(methoxymethyl)benzene were used as coupling partner in the Suzuki reaction. Methyl 5-aminopyridine-2-carboxylate was used in step 4 and General Method L was used prior to SFC as the final step. Purification by chiral SFC in the final step used a Chiralpak AS-H column, 5 μm particle size, 25 cm×10 mm from Daicel on a Minigram SFC instrument from Berger Instruments. In the Table below, “MS r.t.” stands for Mass Spec retention time.

| | | LC/MS | Found | MS | |
|---|---|---|---|---|---|
| Cmpd. No. | Compound Name | (m/z calc.) | M + 1 | r.t. | NMR (shifts in ppm) |
| 178 | rel-5-((2S,3R,4R,5S)- | 499.455 | 500.6 | 3.12 | ¹H NMR (500 MHz, |
| | 3-(4-fluoro-2- | | | | DMSO-d₆) δ 10.64 (s, 1H), |
| | methoxy-3- | | | | 8.81 (d, J = 2.4 Hz, 1H), |
| | (methoxymethyl) | | | | 8.19 (dd, J = 8.6, 2.5 Hz, |
| | phenyl)-4,5-dimethyl- | | | | 1H), 7.98 (d, J = 8.3 Hz, |
| | 5-(trifluoromethyl) | | | | 2H), 7.50 (s, 1H), 7.39 (dd, |
| | tetrahydrofuran-2- | | | | J = 8.8, 6.4 Hz, 1H), 7.05 |
| | carboxamido) | | | | (t, J = 8.9 Hz, 1H), 5.10 (d, |
| | picolinamide | | | | J = 10.5 Hz, 1H), 4.45-4.34 |
| | (first eluting isomer | | | | (m, 2H), 4.30 (dd, J = 10.4, |
| | by SFC) | | | | 7.7 Hz, 1H), 3.80 (s, 3H), |
| | | | | | 3.29 (s, 3H), 2.76 (t, J = |
| | | | | | 7.6 Hz, 1H), 1.62 (s, 3H), |
| | | | | | 0.73 (d, J = 7.4 Hz, 3H) |
| | | | | | ppm. |
| 179 | rel-5-((2R,3S,4S,5R)- | 499.455 | 500.6 | 3.12 | ¹H NMR (500 MHz, |
| | 3-(4-fluoro-2- | | | | DMSO-d₆) δ 10.64 (s, 1H), |
| | methoxy-3- | | | | 8.81 (d, J = 2.6 Hz, 1H), |
| | (methoxymethyl) | | | | 8.19 (dd, J = 8.6, 2.5 Hz, |
| | phenyl)-4,5-dimethyl- | | | | 1H), 8.04-7.93 (m, 2H), |
| | 5-(trifluoromethyl) | | | | 7.51 (s, 1H), 7.39 (dd, J = |
| | tetrahydrofuran-2- | | | | 8.8, 6.4 Hz, 1H), 7.05 (t, J = |
| | carboxamido) | | | | 8.8 Hz, 1H), 5.10 (d, J = |
| | picolinamide | | | | 10.4 Hz, 1H), 4.46-4.35 |
| | (second eluting isomer | | | | (m, 2H), 4.30 (dd, J = 10.4, |
| | by SFC) | | | | 7.7 Hz, 1H), 3.80 (s, 3H), |
| | | | | | 3.29 (s, 3H), 2.76 (p, J = |
| | | | | | 7.7 Hz, 1H), 1.62 (s, 3H), |
| | | | | | 0.75-0.71 (m, 3H) ppm. |

### Example 7

(2R,3S,4S,5R)-3-(3,4-difluoro-2-methoxyphenyl)-4,5-dimethyl-N-(3-methyl-1-(methylsulfonyl)-1H-pyrazol-4-yl)-5-(trifluoromethyl)tetrahydrofuran-2-carboxamide (180)

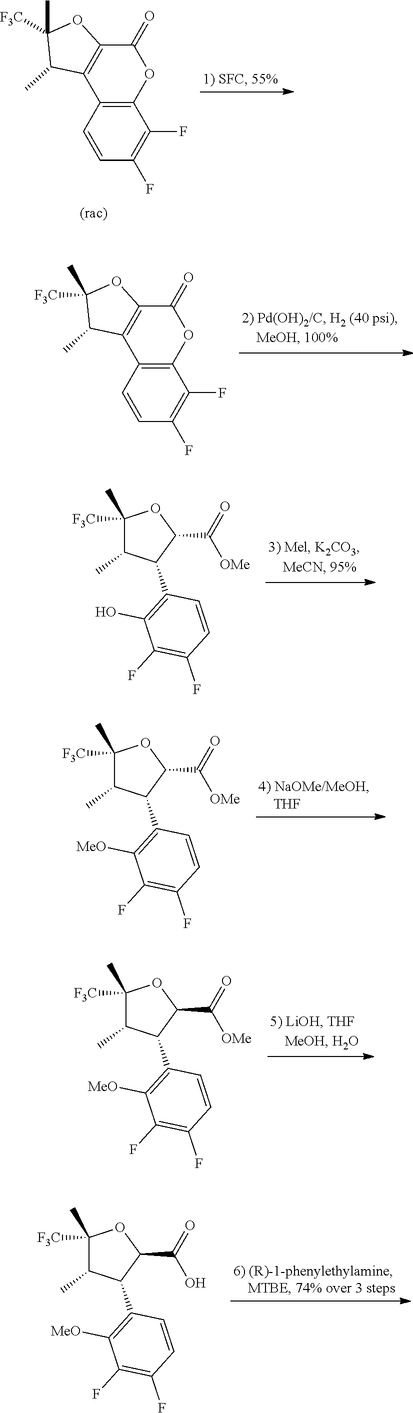 [SMILES: C[C@H]1c2c(c(=O)oc3c(F)c(F)ccc23)O[C@@]1(C)C(F)(F)F ; C[C@H]1c2c(c(=O)oc3c(F)c(F)ccc23)O[C@@]1(C)C(F)(F)F ; COC(=O)[C@H]1O[C@@](C)(C(F)(F)F)[C@@H](C)[C@H]1c1ccc(F)c(F)c1O ; COC(=O)[C@H]1O[C@@](C)(C(F)(F)F)[C@@H](C)[C@H]1c1ccc(F)c(F)c1OC ; COC(=O)[C@@H]1O[C@@](C)(C(F)(F)F)[C@@H](C)[C@H]1c1ccc(F)c(F)c1OC ; COc1c([C@H]2[C@H](C(=O)O)O[C@@](C)(C(F)(F)F)[C@H]2C)ccc(F)c1F]

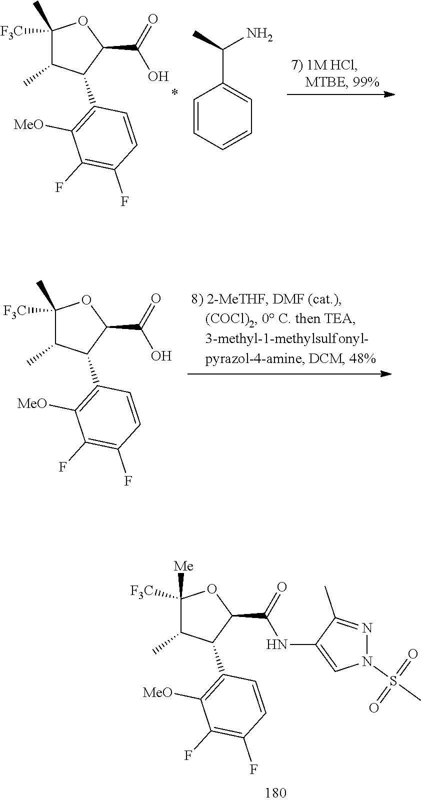

### Step 1:

rac-(1S,2R)-6,7-difluoro-1,2-dimethyl-2-(trifluoromethyl)-1,2-dihydro-4H-furo[2,3-c]chromen-4-one (1348 g, 4.366 mol) was separated by chiral SFC using a (R,R)-Whelk-01 column, 5 μm particle size, 15 cm×3 cm from Regis Technologies on a MultiGram III SFC instrument from Berger Instruments to give:

First Eluting Isomer (rt=1.85 min): (1R,2S)-6,7-difluoro-1,2-dimethyl-2-(trifluoromethyl)-1,2-dihydro-4H-furo[2,3-c]chromen-4-one (only an analytical sample was collected). ¹H NMR (400 MHz, DMSO-d₆) δ 7.57 (ddd, J=9.0, 5.5, 2.0 Hz, 1H), 7.51 (ddd, J=10.3, 9.0, 7.0 Hz, 1H), 4.03 (q, J=7.2 Hz, 1H), 1.65 (s, 3H), 1.45 (dt, J=6.9, 2.2 Hz, 3H) ppm. ESI-MS m/z calc. 320.04718, found 321.3 (M+1)⁺; 319.4 (M−1)⁻.

Second Eluting Isomer (rt=2.38 min): (1S,2R)-6,7-Difluoro-1,2-dimethyl-2-(trifluoromethyl)-1,2-dihydro-4H-furo[2,3-c]chromen-4-one (366.99 g, 26%). ¹H NMR (400 MHz, DMSO-d₆) δ 7.57 (ddd, J=9.0, 5.5, 2.0 Hz, 1H), 7.50 (ddd, J=10.3, 9.0, 7.0 Hz, 1H), 4.03 (q, J=7.2 Hz, 1H), 1.65 (s, 3H), 1.45 (dt, J=6.9, 2.2 Hz, 3H) ppm. ESI-MS m/z calc. 320.04518, found 321.4 (M+1)⁺; 319.4 (M−1)⁻.

### Step 2:

A solution of (1S,2R)-6,7-difluoro-1,2-dimethyl-2-(trifluoromethyl)-1,2-dihydro-4H-furo[2,3-c]chromen-4-one (0.89 kg, 2.78 mol) and 20% palladium hydroxide on carbon (50% wet, 0.39 kg, 0.278 mol) in MeOH (12 L) was stirred under a 40 psi pressure of hydrogen overnight. An increase in the reaction temperature to 37° C. was observed after reacting overnight and the mixture was cooled to 24° C. and hydrogenation was continued for a total of 48 hours. The mixture was filtered through celite, washing with MeOH (20 L) and the filtrate was concentrated in vacuo. The residue was dissolved in toluene (4 L) and concentrated in vacuo, and this process repeated. The residue was dried under vacuum at 40° C. overnight to give methyl (2S,3S,4S,5R)-3-(3,4-difluoro-2-hydroxyphenyl)-4,5-dimethyl-5-(trifluoromethyl)tetrahydrofuran-2-carboxylate (1.0 kg at 91% purity, 100%) as a beige solid. ¹H NMR (400 MHz, DMSO-d₆) 10.20 (br s, 1H), 6.94 (br t, J=7.4 Hz, 1H), 6.79-6.69 (m, 1H), 5.10 (d, J=6.0 Hz, 1H), 4.20 (dd, J=6.1, 8.2 Hz, 1H), 3.43 (s, 3H), 2.94 (quin, J=7.7 Hz, 1H), 1.46 (s, 3H), 0.77 (br d, J=6.8 Hz, 3H) ppm.

### Step 3:

Potassium carbonate (2.0 kg, 14.4 mol) and iodomethane (800 mL, 12.8 mol) were sequentially added to a solution of methyl (2S,3S,4S,5R)-3-(3,4-difluoro-2-hydroxyphenyl)-4,5-dimethyl-5-(trifluoromethyl)tetrahydrofuran-2-carboxylate (1.0 kg, 2.82 mol) in acetonitrile (10 L) under nitrogen stirring at ambient temperature. After stirring overnight, additional iodomethane (120 mL, 2 mmol) was added. After stirring overnight, additional iodomethane (60 mL, 0.85 mmol) was added and the mixture was stirred for 3 days. The reaction mixture was diluted with MTBE (30 L), treated with celite (1 kg) and filtered through a bed of celite (1 kg) washing with MTBE (10 L). The filtrate was filtered a second time through celite (1 kg) washing with MTBE (4 L) and the filtrate concentrated in vacuo. The residue was dissolved in toluene (4 L) and concentrated in vacuo, and this process repeated. The residue was dried under vacuum at 40° C. overnight to give methyl (2S,3S,4S,5R)-3-(3,4-difluoro-2-methoxyphenyl)-4,5-dimethyl-5-(trifluoromethyl)tetrahydrofuran-2-carboxylate (0.99 kg at 90% purity, 95%) as a brown solid. ¹H NMR (400 MHz, DMSO-d₆) 7.14-7.00 (m, 2H), 5.14 (d, J=6.0 Hz, 1H), 4.15 (dd, J=6.2, 8.4 Hz, 1H), 3.88 (d, J=1.7 Hz, 3H), 2.97 (quin, J=7.8 Hz, 1H), 1.48 (s, 3H), 0.72 (br d, J=6.6 Hz, 3H) ppm.

### Step 4 and 5:

Sodium methoxide (25% in methanol, 65 mL, 0.28 mol) was added to a solution of methyl (2S,3S,4S,5R)-3-(3,4-difluoro-2-methoxyphenyl)-4,5-dimethyl-5-(trifluoromethyl) tetrahydrofuran-2-carboxylate (0.98 kg, 2.66 mol) in THF (10 L) stirring at ambient temperature under nitrogen. After 5 hours, MeOH (1 L), water (1 L) and lithium hydroxide monohydrate (0.168 kg, 4.0 mol) were sequentially added and the mixture was stirred overnight. The reaction mixture was poured into 1M HCl (4.4 L, 4.4 mol) then extracted with MTBE (20 L). The aqueous layer was further extracted with MTBE (2×5 L) and the combined organic layers washed with brine (2 L), dried (Na₂SO₄) then treated with activated carbon (50 g, 5% w/w) with stirring for 1 h. The mixture was filtered through celite, washing with MTBE (2×4 L) and the filtrate concentrated in vacuo. The residue was dissolved in toluene (4 L) and concentrated in vacuo, then dissolved in MTBE (4 L) and concentrated in vacuo again to give (2R,3S,4S,5R)-3-(3,4-difluoro-2-methoxyphenyl)-4,5-dimethyl-5-(trifluoromethyl)tetrahydrofuran-2-carboxylic acid (1.06 kg at 77.7% purity) as an amber oil, which was used without further purification.

### Step 6:

Crude (2R,3S,4S,5R)-3-(3,4-difluoro-2-methoxyphenyl)-4,5-dimethyl-5-(trifluoromethyl)tetrahydrofuran-2-carboxylic acid (2.09 kg at 77% purity, 4.54 mol) was dissolved in MTBE (25 L) in a 100 L Chemglass reactor then stirred at 84 rpm at ambient temperature. A mixture of (R)-1-phenylethylamine (0.704 kg, 5.81 mol) and MTBE (2 L) was added to the reactor, followed by additional MTBE to give a total volume of 30 L in the reactor. After 2 hours additional MTBE (2 L) was added to the reaction and after a total of 3.5 hours the mixture was filtered, washing with MTBE (2 L). The reactor was rinsed with MTBE (4 L), which was used to rinse the solids, which were then compressed and dried on the Buchner funnel for 2 hours. The solid product cake was loosened then dried under a stream of nitrogen and under vacuum overnight on the Buchner funnel. The isolated solids were dried in a convection oven at 40° C. for 24 hours to give (2R,3S,4S,5R)-3-(3,4-Difluoro-2-methoxyphenyl)-4,5-dimethyl-5-(trifluoromethyl)tetrahydrofuran-2-carboxylic acid (R)-1-phenylethan-1-amine salt (1.86 kg at 95.7% purity, 74% over 3 steps) as an off-white solid. ¹H NMR, 400 MHz, DMSO-d₆) 8.34 (br s, 2H), 7.46-7.41 (m, 2H), 7.36-7.27 (m, 3H), 7.16-7.11 (m, 1H), 7.10-7.03 (m, 1H), 4.58 (d, J=9.9 Hz, 1H), 4.23 (q, J=6.7 Hz, 1H), 3.99 (dd, J=7.8, 9.8 Hz, 1H), 3.90 (d, J=2.0 Hz, 3H), 2.60 (quin, J=7.5 Hz, 1H), 1.50 (s, 3H), 1.40 (d, J=6.7 Hz, 3H), 0.71-0.59 (m, 3H) ppm.

### Step 7:

To a suspension of (2R,3S,4S,5R)-3-(3,4-difluoro-2-methoxy-phenyl)-4,5-dimethyl-5-(trifluoromethyl)tetrahydrofuran-2-carboxylic acid (1R)-1-phenylethanamine salt (10.6 g, 22.29 mmol) in MTBE (250 mL) was added HCl (200 mL of 2 M, 400.0 mmol). The layers were separated and the organic layer was washed with water (200 mL), dried (MgSO₄), filtered and concentrated in vacuo to give (2R,3S,4S,5R)-3-(3,4-difluoro-2-methoxy-phenyl)-4,5-dimethyl-5-(trifluoromethyl)tetrahydrofuran-2-carboxylic acid (8.4 g, 99%) as an oil. ¹H NMR (400 MHz, Chloroform-d) δ 6.96 (ddd, J=7.9, 5.6, 2.0 Hz, 1H), 6.88 (td, J=9.2, 7.3 Hz, 1H), 4.96 (d, J=10.5 Hz, 1H), 4.15 (dd, J=10.5, 8.0 Hz, 1H), 4.02 (d, J=2.8 Hz, 3H), 2.74 (p, J=7.6 Hz, 1H), 1.64 (t, J=1.2 Hz, 3H), 0.79 (dq, J=7.4, 2.3 Hz, 3H) ppm.

### Step 8:

One drop of DMF (5 μL, 0.06457 mmol) was added to a solution of (2R,3S,4S,5R)-3-(3,4-difluoro-2-methoxy-phenyl)-4,5-dimethyl-5-(trifluoromethyl)tetrahydrofuran-2-carboxylic acid (62 mg, 0.1750 mmol) in DCM (1.1 mL) at 0° C., followed by a dropwise addition of oxalyl chloride (50 μL, 0.5732 mmol). The reaction mixture was warmed to ambient temperature and stirred for 45 minutes before being concentrated in vacuo. The resulting residue was dissolved in DCM (1 mL) and added dropwise to a solution of 3-methyl-1-methylsulfonyl-pyrazol-4-amine (42 mg, 0.2397 mmol) and TEA (75 μL, 0.5381 mmol) in DCM (1 mL) at 0° C. The reaction mixture was allowed to warm to ambient temperature and stirred for 2 hours. The reaction mixture was quenched with water (5 mL) and partitioned with ethyl acetate (10 mL). The layers were separated and the organic phase was washed with brine (5 mL), dried (sodium sulfate), filtered and concentrated under reduced pressure. Purification via flash column chromatography (4 g SiO₂, eluting with 0 to 40% ethyl acetate in heptane) gave (2R,3S,4S,5R)-3-(3,4-difluoro-2-methoxyphenyl)-4,5-dimethyl-N-(3-methyl-1-(methylsulfonyl)-1H-pyrazol-4-yl)-5-(trifluoromethyl)tetrahydrofuran-2-carboxamide (180, 44.3 mg, 48%) as a white solid. ¹H NMR (400 MHz, Chloroform-d) δ 8.50 (t, J=0.5 Hz, 1H), 8.11 (s, 1H), 7.09 (ddd, J=8.2, 5.6, 2.2 Hz, 1H), 6.90 (td, J=9.2, 7.5 Hz, 1H), 5.05 (d, J=11.0 Hz, 1H), 4.06 (dd, J=11.0, 7.8 Hz, 1H), 4.00 (d, J=2.8 Hz, 3H), 3.21 (s, 3H), 2.76 (p, J=7.6 Hz, 1H), 2.33 (d, J=0.5 Hz, 3H), 1.67 (d, J=1.1 Hz, 3H), 0.79 (dt, J=7.5, 2.4 Hz, 3H) ppm. ESI-MS m/z calc. 511.12003, found 512.5 (M+1)⁺; 510.5 (M−1)⁻; Retention time: 3.4 minutes.

The following compounds were made using a similar method to that described in Example 7, except that different coupling partners were used in the amide coupling step 8. For step 8, DCM can typically be substituted for 2-MeTHF and Et₃N substituted with DIPEA or K₂CO₃. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 181 | (2R,3S,4S,5R)-3-(3,4- | 529.5 | 530.8 | 3.29 | ¹H NMR (500 MHz, DMSO-d₆) δ |
| | difluoro-2- | | | | 10.55 (s, 1H), 8.34 (d, J = 5.6 |
| | methoxyphenyl)-4,5- | | | | Hz, 1H), 7.67 (d, J = 2.1 Hz, |
| | dimethyl-N-(2- | | | | 1H), 7.52 (dd, J = 5.6, 2.1 Hz, |
| | (morpholinomethyl) | | | | 1H), 7.22-7.08 (m, 2H), 5.08 |
| | pyridin-4-yl)-5- | | | | (d, J = 10.2 Hz, 1H), 4.25 (dd, |
| | (trifluoromethyl) | | | | J = 10.3, 7.7 Hz, 1H), 3.95 (d, |
| | tetrahydrofuran-2- | | | | J = 2.2 Hz, 3H), 3.58 (t, J = |
| | carboxamide | | | | 4.6 Hz, 4H), 3.51 (d, J = 2.7 Hz, |
| | | | | | 2H), 2.81-2.73 (m, 1H), 2.39 |
| | | | | | (t, J = 4.6 Hz, 4H), 1.59 (s, 3H), |
| | | | | | 0.76-0.67 (m, 3H) ppm. |
| 182 | (2R,3S,4S,5R)-3-(3,4- | 549.481 | 550.8 | 3.63 | ¹H NMR (500 MHz, DMSO-d₆) δ |
| | difluoro-2- | | | | 10.57 (s, 1H), 8.35 (d, J = 5.6 |
| | methoxyphenyl)-N- | | | | Hz, 1H), 7.64 (d, J = 2.1 Hz, |
| | (2-((3,3- | | | | 1H), 7.53 (dd, J = 5.6, 2.1 Hz, |
| | difluoropyrrolidin-1- | | | | 1H), 7.21-7.08 (m, 2H), 5.08 |
| | yl)methyl)pyridin-4- | | | | (d, J = 10.2 Hz, 1H), 4.25 (dd, |
| | yl)-4,5-dimethyl-5- | | | | J = 10.3, 7.7 Hz, 1H), 3.95 (d, |
| | (trifluoromethyl) | | | | J = 2.0 Hz, 3H), 3.68 (d, J = 2.6 |
| | tetrahydrofuran-2- | | | | Hz, 2H), 2.92 (t, J = 13.4 Hz, 2H), |
| | carboxamide | | | | 2.75 (dt, J = 13.8, 7.3 Hz, 3H), |
| | | | | | 2.25 (tt, J = 14.9, 6.9 Hz, 2H), |
| | | | | | 1.59 (s, 3H), 0.77-0.67 (m, 3H) |
| | | | | | ppm. |
| 183 | (2R,3S,4S,5R)-3-(3,4- | 487.463 | 488.6 | 2.8 | ¹H NMR (500 MHz, DMSO-d₆) δ |
| | difluoro-2- | | | | 10.52 (s, 1H), 8.33 (dd, J = |
| | methoxyphenyl)-N-(2- | | | | 5.6, 0.6 Hz, 1H), 7.73-7.68 (m, |
| | ((dimethylamino) | | | | 1H), 7.48 (dd, J = 5.6, 2.1 Hz, |
| | methyl)pyridin-4-yl)- | | | | 1H), 7.21-7.08 (m, 2H), 5.07 |
| | 4,5-dimethyl-5- | | | | (d, J = 10.3 Hz, 1H), 4.24 (dd, |
| | (trifluoromethyl) | | | | J = 10.3, 7.6 Hz, 1H), 3.95 (d, J = |
| | tetrahydrofuran-2- | | | | 2.2 Hz, 3H), 3.44 (s, 2H), 2.82-2.70 |
| | carboxamide | | | | (m, 1H), 2.17 (s, 6H), 1.60 (s, 3H), |
| | | | | | 0.76-0.66 (m, 3H) ppm. |
| 184 | (2R,3S,4S,5R)-3-(3,4- | 513.5 | 514.7 | 2.86 | ¹H NMR (500 MHz, DMSO-d₆) δ |
| | difluoro-2- | | | | 10.53 (s, 1H), 8.32 (dd, J = 5.6 |
| | methoxyphenyl)-4,5- | | | | Hz, 1H), 7.65 (d, J = 2.1 Hz, 1H), |
| | dimethyl-N-(2- | | | | 7.51 (dd, J = 5.5, 2.1 Hz, 1H), |
| | (pyrrolidin-1- | | | | 7.25-7.07 (m, 2H), 5.07 (d, J = 10.3 |
| | ylmethyl)pyridin-4- | | | | Hz, 1H), 4.24 (dd, J = 10.3, 7.7 Hz, |
| | yl)-5- | | | | 1H), 3.95 (d, J = 2.1 Hz, 3H), |
| | (trifluoromethyl) | | | | 3.69-3.55 (m, 2H), 2.81-2.72 (m, |
| | tetrahydrofuran-2- | | | | 1H), 2.48-2.40 (m, 4H), 1.73-1.66 |
| | carboxamide | | | | (m, 4H), 1.59 (s, 3H), 0.78-0.66 |
| | | | | | (m, 3H) ppm. |
| 185 | (2R,3S,4S,5R)-3-(3,4- | 483.431 | 484.2 | 3.21 | ¹H NMR (500 MHz, DMSO-d₆) δ |
| | difluoro-2- | | | | 10.20 (s, 1H), 8.13 (s, 1H), |
| | methoxyphenyl)-4,5- | | | | 7.96 (d, J = 1.9 Hz, 1H), 7.48 (d, |
| | dimethyl-N-(1-methyl- | | | | J = 8.7 Hz, 1H), 7.41 (dd, J = 8.7, |
| | 1H-benzo[d]imidazol- | | | | 1.9 Hz, 1H), 7.23-7.11 (m, 2H), |
| | 6-yl)-5- | | | | 5.07 (d, J = 10.4 Hz, 1H), |
| | (trifluoromethyl) | | | | 4.27 (dd, J = 10.4, 7.6 Hz, 1H), |
| | tetrahydrofuran-2- | | | | 3.96 (d, J = 2.0 Hz, 3H), 3.80 (s, |
| | carboxamide | | | | 3H), 2.77 (p, J = 7.5 Hz, 1H), |
| | | | | | 1.61 (s, 3H), 0.80-0.68 (m, 3H) ppm. |
| 186 | (2R,3S,4S,5R)-3-(3,4- | 515.473 | 515.9 | 3.49 | ¹H NMR (400 MHz, Methanol-d4) δ |
| | difluoro-2- | | | | 8.26 (dd, J = 2.7, 0.7 Hz, 1H), |
| | methoxyphenyl)-4,5- | | | | 7.76 (dd, J = 9.1, 2.7 Hz, 1H), |
| | dimethyl-N-(1-methyl- | | | | 7.13 (ddd, J = 8.2, 5.6, 2.2 Hz, 1H), |
| | 1H-benzo[d]imidazol- | | | | 6.97 (ddd, J = 9.9, 8.9, 7.5 Hz, 1H), |
| | 6-yl)-5- | | | | 6.79 (dd, J = 9.2, 0.8 Hz, 1H), 5.03 |
| | (trifluoromethyl) | | | | (d, J = 10.6 Hz, 1H), 4.28 (dd, J = |
| | tetrahydrofuran-2- | | | | 10.6, 8.0 Hz, 1H), 3.99 (d, J = 2.3 Hz, |
| | carboxamide | | | | 3H), 3.81-3.73 (m, 4H), 3.47-3.39 |
| | | | | | (m, 4H), 2.78 (p, J = 7.7 Hz, 1H), |
| | | | | | 1.66 (d, J = 1.1 Hz, 3H), 0.81 |
| | | | | | (dq, J = 7.3, 2.3 Hz, 3H) ppm. |
| 187 | (2R,2S,4S,5R)-N-(5- | 455.378 | 456.2 | 3.6 | ¹H NMR (500 MHz, DMSO-d₆) δ |
| | cyanopyridin-2-yl)-3- | | | | 11.13 (s, 1H), 8.81 (dd, J = |
| | (3,4-difluoro-2- | | | | 2.3, 0.9 Hz, 1H), 8.25 (dd, J = |
| | methoxyphenyl)-4,5- | | | | 8.8, 2.3 Hz, 1H), 8.17 (dd, J = 8.8, |
| | dimethyl-5- | | | | 0.9 Hz, 1H), 7.24-7.12 (m, 2H), |
| | (trifluoromethyl) | | | | 5.26 (d, J = 10.4 Hz, 1H), 4.26 |
| | tetrahydrofuran-2- | | | | (dd, J = 10.4, 7.6 Hz, 1H), 3.94 |
| | carboxamide | | | | (d, J = 2.0 Hz, 3H), 2.77 (p, J = |
| | | | | | 7.5 Hz, 1H), 1.60 (s, 3H), 0.71 (d, |
| | | | | | J = 7.5 Hz, 3H) ppm. |
| 188 | (2R,3S,4S,5R)-3-(3,4- | 509.447 | 510 | 33.24 | ¹H NMR (500 MHz, DMSO-d₆) δ |
| | difluoro-2- | | | | 10.87 (s, 1H), 8.56 (d, J = 5.5 Hz, |
| | methoxyphenyl)-4,5- | | | | 1H), 8.26 (d, J = 2.0 Hz, 1H), |
| | dimethyl-N-(2- | | | | 7.95-7.71 (m, 1H), 7.40 (s, 2H), |
| | sulfamoylpyridin-4- | | | | 7.21-7.12 (m, 2H), 5.12 (d, J = 10.2 |
| | yl)-5- | | | | Hz, 1H), 4.26 (dd, J = 10.2, 7.7 Hz, |
| | (trifluoromethyl) | | | | 1H), 3.96 (d, J = 2.0 Hz, 3H), 2.78 |
| | tetrahydrofuran-2- | | | | (p, J = 7.6 Hz, 1H), 1.61 (s, 3H), |
| | carboxamide | | | | 0.84-0.60 (m, 3H) ppm. |
| 189 | (2R,3S,4S,5R)-3-(3,4- | 496.375 | 497 | 3.82 | ¹H NMR (500 MHz, DMSO-d₆) δ |
| | difluoro-2- | | | | 10.74 (s, 1H), 8.14 (d, J = 5.7 |
| | methoxyphenyl)-N-(2- | | | | Hz, 1H), 7.66 (t, J = 73.0 Hz, 1H), |
| | (difluoromethoxy) | | | | 7.43 (dd, J = 5.7, 1.8 Hz, 1H), 7.35 |
| | pyridin-4-yl)-4,5- | | | | (d, J = 1.7 Hz, 1H), 7.23-7.10 (m, 2H), |
| | dimethyl-5- | | | | 5.10 (d, J = 10.1 Hz, 1H), 4.26 (dd, |
| | (trifluoromethyl) | | | | J = 10.1, 7.7 Hz, 1H), 3.95 (d, J = |
| | tetrahydrofuran-2- | | | | 2.0 Hz, 3H), 2.78 (p, J = 7.5 Hz, 1H), |
| | carboxamide | | | | 1.60 (s, 3H), 0.85-0.61 (m, 3H) ppm. |
| 190 | (2R,3S,4S,5R)-3,4- | 446.368 | 447 | 2.98 | ¹H NMR (500 MHz, DMSO-d₆) δ |
| | difluoro-2- | | | | 11.36 (s, 1H), 9.96 (s, 1H), |
| | methoxyphenyl)-4,5- | | | | 7.81 (d, J = 2.9 Hz, 1H), 7.47 |
| | dimethyl-N-(6-oxo- | | | | (dd, J = 9.7, 2.9 Hz, 1H), 7.25-7.06 |
| | 1,6-dihydropyridin-3- | | | | (m, 2H), 6.33 (d, J = 9.7 Hz, 1H), |
| | yl)-5- | | | | 5.00 (d, J = 10.4 Hz, 1H), 4.19 (dd, |
| | (trifluoromethyl) | | | | J = 10.4, 7.6 Hz, 1H), 3.95 (d, J = |
| | tetrahydrofuran-2- | | | | 1.9 Hz, 3H), 2.74 (p, J = 7.5 Hz, 1H), |
| | carboxamide | | | | 1.59 (s, 3H), 0.72 (dd, J = 7.4, 2.4 |
| | | | | | Hz, 3H) ppm. |
| 191 | (2R,3S,4S,5R)-N-(2- | 523.291 | 523.09 | 1.16 | ¹H NMR (400 MHz, chloroform-d) δ |
| | bromo-5- | | | | 8.59 (s, 1H), 8.42 (s, 1H), |
| | methylpyridin-4-yl)-3- | | | | 8.12 (s, 1H), 7.11-7.07 (m, 1H), 6.91 |
| | (3,4-difluoro-2- | | | | (td, J = 9.2, 7.3 Hz, 1H), 5.04 (d, J = |
| | methooxyphenyl)-4,5- | | | | 11.4 Hz, 1H), 4.06 (dd, J = 11.0, 7.8 |
| | dimethyl-5- | | | | Hz, 1H), 4.01 (t, J = 2.5 Hz, 3H), |
| | (trifluoromethyl) | | | | 2.76 (p, J = 7.6 Hz, 1H), 2.22 (s, 3H), |
| | tetrahydrofuran-2- | | | | 1.67 (s, 3H), 0.79 (td, J = 4.8, 2.4 |
| | carboxamide | | | | Hz, 3H) ppm. |
| 192 | (2R,3S,4S,5R)-N-(6- | 455.378 | 456 | 3.59 | ¹H NMR (500 MHz, DMSO-d₆) δ |
| | cyanopyridin-3-yl)-3- | | | | 10.79 (s, 1H), 8.92 (d, J = 2.4 |
| | (3,4-difluoro-2- | | | | Hz, 1H), 8.29 (dd, J = 8.6, 2.5 |
| | methoxyphenyl)-4,5- | | | | Hz, 1H), 7.98 (d, J = 8.5 Hz, 1H), |
| | dimethyl-5- | | | | 7.17 (dd, J = 8.5, 4.8 Hz, 2H), |
| | (trifluoromethyl) | | | | 5.14 (d, J = 10.1 Hz, 1H), 4.27 (dd, |
| | tetrahydrofuran-2- | | | | J = 10.1, 7.7 Hz, 1H), 3.95 (d, |
| | carboxamide | | | | J = 2.0 Hz, 3H), 2.78 (p, J = 7.6 Hz, |
| | | | | | 1H), 1.61 (s, 3H), 0.74 ( |
| | | | | | dd, J = 7.4, 2.4 Hz, 3H) ppm. |
| 193 | (2R,3S,4S,5R)-N-(6- | | | | ¹H NMR (500 MHz, DMSO-d₆) δ |
| | (1H-imidazol-2- | | | | 12.68 (s, 1H), 10.51 (s, 1H), |
| | yl)pyridin-3-yl)-3- | | | | 8.85 (d, J = 2.4 Hz, 1H), 8.10 |
| | (3,4-difluoro-2- | | | | (dd, J = 88.7, 2.5 Hz, 1H), 7.99 |
| | methoxyphenyl)-4,5- | | | | (d, J = 8.5 Hz, 1H), 7.24-7.11 (m, 3H), |
| | dimethyl-5- | | | | 7.05 (s, 1H), 5.12 (d, J = 10.3 Hz, 1H), |
| | (trifluoromethyl) | | | | 4.28 (dd, J = 10.4, 7.7 Hz, 1H), 3.97 |
| | tetrahydrofuran-2- | | | | (d, J = 2.0 Hz, 3H), 2.78 (p, J = 7.6 Hz, |
| | carboxamide | | | | 1H), 1.62 (s, 3H), 0.81-0.68 (m, 3H) . |
| | | | | | ppm |
| 194 | (2R,3S,4S,5R)-3-(3,4- | 536.512 | 537.08 | 3.23 | ¹H NMR (500 MHz, DMSO-d₆) δ |
| | difluoro-2- | | | | 10.40 (s, 1H), 8.70 (dd, J = 2.6, 0.7 |
| | methoxyphenyl)-4,5- | | | | Hz, 1H), 7.98 (dd, J = 8.4, 2.6 Hz, 1H), |
| | dimethyl-N-(6-(2- | | | | 7.33 (d, J = 8.5 Hz, 1H), 7.23-7.11 |
| | (methylsuflonyl) | | | | (m, 2H), 5.09 (d, J = 10.3 Hz, 1H), 4.25 |
| | ethyl)pyridin-3-yl)-5- | | | | (dd, J = 10.3, 7.6 Hz, 1H), 3.95 (d, J = |
| | (trifluoromethyl) | | | | 2.2 Hz, 3H), 3.54-3.45 (m, 2H), |
| | tetrahydrofuran-2- | | | | 3.17-3.09 (m, 2H), 2.99 (s, 3H), 2.77 |
| | carboxamide | | | | (p, J = 7.5 Hz, 1H), 1.61 (s, 3H), 0.73 |
| | | | | | (dt, J = 7.7, 2.5 Hz, 3H) ppm. |
| 195 | (2R,3S,4S,5R)-3-(3,4- | 530.484 | 532 | 3.69 | ¹H NMR (500 MHz, DMSO-d₆) δ |
| | difluoro-2- | | | | 10.48 (s, 1H), 8.01 (d, J = 5.5 |
| | methoxyphenyl)-4,5- | | | | Hz, 1H), 7.20-7.14 (m, 1H), |
| | dimethyl-N-(2- | | | | 7.10 (d, J = 5.8 Hz, 3H), 5.13 (tt, |
| | ((tetrahydro-2H-pyran- | | | | J = 8.5, 4.1 Hz, 1H), 5.06 (d, J = |
| | 4-yl)oxy)pyridin-4-yl)- | | | | 10.2 Hz, 1H), 4.23 (dd, J = 10.2, |
| | 5-(trifluoromethyl) | | | | 7.7 Hz, 1H), 3.94 (d, J = 2.0 Hz, |
| | tetrahydrofuran-2- | | | | 3H), 3.87-3.78 (m, 2H), 3.47 (t, J = |
| | carboxamide | | | | 9.6 Hz, 2H), 2.76 (p, J = 7.7 Hz, 1H), |
| | | | | | 1.98-1.88 (m, 2H), 1.64-1.53 (m, 5H), |
| | | | | | 0.72 (d, J = 7.4 Hz, 3H) ppm. |
| 196 | (2R,3S,4S,5R)-3-(3,4- | 420.331 | 422 | 3.24 | ¹H NMR (500 MHz, DMSO-d₆) |
| | difluoro-2- | | | | δ 11.44 (s, 1H), 7.88 (s, 1H), |
| | methoxyphenyl)-4,5- | | | | 7.21-7.15 (m, 1H), 7.15-7.12 (m, 1H), |
| | dimethyl-N-(oxazol-2- | | | | 7.11 (s, 1H), 5.08 (d, J = 10.4 Hz, 1H), |
| | yl)-5- | | | | 4.20 (dd, J = 10.4, 7.8 Hz, 1H), 3.95 |
| | (trifluoromethyl) | | | | (d, J = 2.1 Hz, 3H), 2.75 (p, J = 7.6 Hz, |
| | tetrahydrofuran-2- | | | | 1H), 1.59 (s, 3H), 0.80-0.63 (m, 3H) |
| | carboxamide | | | | ppm. |

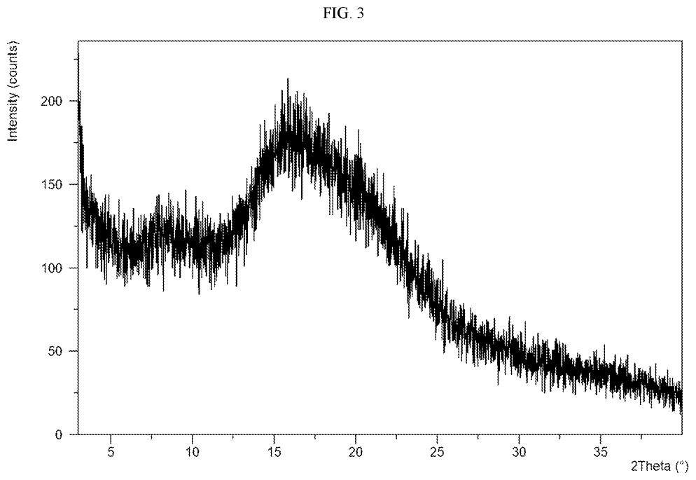

Compound 183 was analyzed by X-ray powder diffraction and determined to be amorphous (see FIG. 3).

The following compounds were made using the method described in Example 7, except that rac-3-[(4-amino-2-pyridyl)oxy]-1-methyl-pyrrolidin-2-one was used in the amide coupling step 8 and the diastereomeric products generated were separated by chiral SFC using a Chiralpak IA column, 5 μm particle size, 25 cm×10 mm from Daicel on a Minigram SFC instrument from Berger Instruments. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 197 | rel-(2R*,3S*,4S*,5R*)- | 543.483 | 544 | 3.39 | ¹H NMR (500 MHz, DMSO-d₆) δ |
| | 3-(3,4-difluoro-2- | | | | 10.55 (s, 1H), 8.02 (d, J = 5.8 |
| | methoxyphenyl)-4,5- | | | | Hz, 1H), 7.21-7.07 (m, 4H), |
| | dimethyl-N-(2-((1- | | | | 5.52 (t, J = 8.0 Hz, 1H), 5.07 |
| | methyl-2-oxopyrrolidin- | | | | (d, J = 10.1 Hz, 1H), 4.24 (dd, |
| | 3-yl)oxy)pyridin-4-yl)- | | | | J = 10.1, 7.7 Hz, 1H), 3.94 (d, |
| | 5-(trifluoromethyl) | | | | J = 2.0 Hz, 3H), 3.39-3.31 (m, 2H), |
| | tetrahydrofuran-2- | | | | 2.81-2.70 (m, 4H), 2.56-2.51 |
| | carboxamide | | | | (m, 1H), 1.91-1.78 (m, 1H), |
| | (first eluting isomer | | | | 1.59 (s, 3H), 0.73 (d, J = 7.4 Hz, |
| | by SFC) | | | | 3H) ppm. |
| 198 | rel-(2R*,3S*,4S*,5R*)- | 543.483 | 544 | 3.39 | ¹H NMR (500 MHz, DMSO-d₆) |
| | 3-(3,4-difluoro-2- | | | | δ 10.54 (s, 1H), 8.02 (d, J = |
| | methoxyphenyl)-4,5- | | | | 5.7 Hz, 1H), 7.23-7.07 (m, 4H), |
| | dimethyl-N-(2-((1- | | | | 5.52 (t, J = 7.9 Hz, 1H), 5.07 |
| | methyl-2-oxopyrrolidin- | | | | (d, J = 10.1 Hz, 1H), 4.24 (dd, |
| | 3-yl)oxy)pyridin-4-yl)- | | | | J = 10.1, 7.7 Hz, 1H), 3.94 (d, J = |
| | 5-(trifluoromethyl) | | | | 2.1 Hz, 3H), 3.40-3.32 (m, 2H), |
| | tetrahydrofuran-2- | | | | 2..80-2.70 (m, 4H), 2.56-2.51 |
| | carboxamide | | | | (m, 1H), 1.89-1.80 (m, 1H), 1.59 |
| | | | | | (s, 3H), 0.73 (d, J = 7.4 Hz, 3H) ppm. |

The following compounds were made using the method described in Example 7, except that different coupling partners were used in the amide coupling step 8 and General Method B was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 199 | rel-(2R*,3S*,4S*,5R*)-3- | 508.411 | 509.6 | 3.14 | ¹H NMR (500 MHz, |
| | (3,4-difluoro-2- | | | | Chloroform-d) δ 8.95 (s, 1H), |
| | methoxyphenyl)-N-(2- | | | | 8.45-8.40 (m, 2H), 7.10-7.05 |
| | (-1,2-dihydroxyethyl)-5- | | | | (m, 1H), 6.91 (td, J = 9.1, 7.3 |
| | fluoropyridin-4-yl)-4,5- | | | | Hz, 1H), 5.05 (d, J = 11.1 Hz, |
| | dimethyl-5- | | | | 1H), 4.80-4.74 (m, 1H), 4.09 |
| | (trifluoromethyl) | | | | (dd, J = 1.11, 7.9 Hz, 1H), 4.01 |
| | tetrahydrofuran-2- | | | | (d, J = 2.9 Hz, 3H), 3.91 (dd, |
| | carboxamide | | | | J = 11.5, 3.8 Hz, 1H), 3.74 (dd, |
| | (using first eluting isomer | | | | J = 11.4, 5.2 Hz, 1H), 2.77 (p, |
| | of 2-(2,2-dimethyl-1,3- | | | | J = 7.6 Hz, 1H), 1.69 (s, 3H), |
| | dioxolan-4-yl)-5-fluoro- | | | | 0.80 (dt, J = 7.3, 2.4 Hz, 3H) |
| | pyridin-4-amine by SFC | | | | ppm. |
| | in step 8) | | | | |
| 200 | rel-(2R*,3S*,4S*,5R*)-3- | 508.411 | 509.6 | 3.14 | ¹H NMR (500 MHz, |
| | (3,4-difluoro-2- | | | | Chloroform-d) δ 8.96 (s, 1H), |
| | methoxyphenyl)-N-(2- | | | | 8.45-8.42 (m, 2H), 7.10-7.05 |
| | (1,2-dihydroxyethyl)-5- | | | | (m, 1H), 6.95-6.88 (m, 1H), |
| | fluoropyridin-4-yl)-4,5- | | | | 5.06 (d, J = 11.1 Hz, 1H), |
| | dimethyl-5- | | | | 4.79 (t, J = 4.4 Hz, 1H), 4.09 |
| | (trifluoromethyl) | | | | (dd, J = 11.1, 7.8 Hz, 1H), 4.02 |
| | tetrahydrofuran-2- | | | | (d, J = 3.0 Hz, 3H), 3.93 (dd, |
| | carboxamide | | | | J = 11.5, 3.7 Hz, 1H), 3.74 |
| | (using second eluting | | | | (dd, J = 11.6, 5.0 Hz, 1H), |
| | isomer of 2-(2,2-dimethyl- | | | | 2.77 (t, J = 7.6 Hz, 1H), 1.69 |
| | 1,3-dioxolan-4-yl)-5- | | | | (s, 3H), 0.82-0.76 (m, 3H) ppm. |
| | fluoro-pyridn-4-amine | | | | |
| | by SFC in step 8) | | | | |
| 201 | (2R,3S,4S,5R)-3-(3,4- | 507.451 | 508.433 | 2.97 | ¹H NMR (500 MHz, |
| | difluoro-2- | | | | DMSO-d₆) δ 9.67 (s, 1H), 7.84 |
| | methoxyphenyl)- | | | | (s, 1H), 7.24-7.10 (m, 2H), 5.13 (d, |
| | N-(1-((R)-2,3- | | | | J = 10.6 Hz, 1H), 4.85 (s, 1H), |
| | dihydroxypropyl)-3-methyl- | | | | 4.65 (s, 1H), 4.21 (dd, J = 10.6, |
| | 1H-pyrazol-4-yl)-4,5- | | | | 7.5 Hz, 1H), 4.04 (dd, J = 13.7, |
| | dimethyl-5- | | | | 4.1 Hz, 1H), 3.95 (d, J = 2.0 Hz, |
| | (trifluoromethyl) | | | | 3H), 3.81 (dd, J = 13.8, 7.6 Hz, |
| | tetrahydrofuran-2- | | | | 1H), 3.71 (d, J = 6.6 Hz, 1H), |
| | carboxamide | | | | 3.25 (s, 1H), 2.74 (p, J = 7.5 Hz, |
| | (using 1-[[(4R)-2,2- | | | | 1H), 2.06 (s, 3H), 1.58 (s, 3H), |
| | dimethyl-1,3-dioxolan-4-yl] | | | | 0.76-0.61 (m, 3H) ppm. |
| | methyl]-3-methyl-pyrazol- | | | | |
| | 4-amine in step 8) | | | | |

The following compounds were made using the method described in Example 7, except that different coupling partners were used in the amide coupling step 8. Diastereomers generated in step 8 were separated by chiral SFC and deprotection using General Method B was carried out as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 202 | rel-(2R*,3S*,4S*,5R*)-3- | 504.447 | | 3.13 | ¹H NMR (400 MHz, DMSO-d₆ δ |
| | (3,4-difluoro-2- | | | | 10.56 (s, 1H), 8.35 (d, J = 5.4 |
| | methoxyphenyl)-N-(2-(1,2- | | | | Hz, 1H), 7.85-7.82 (m, 1H), 7.52 |
| | dihydroxypropan-2- | | | | (dd, J = 5.6, 2.1 Hz, 1H), 7.24-7.06 |
| | yl)pyridin-4-yl)-4,5- | | | | (m, 2H), 5.09 (d, J = 10.5 Hz, 1H), |
| | dimethyl-5- | | | | 5.05 (s, 1H), 4.58 (t, J = 6.0 Hz, |
| | (trifluoromethyl) | | | | 1H), 4.25 (dd, J = 10.3, 7.9 Hz, 1H), |
| | tetrahydrofuran-2- | | | | 3.96 (d, J = 2.3 Hz, 3H), 3.51 (d, |
| | carboxamide | | | | J = 6.6 Hz, 2H), 3.29 (d, J = 0.9 |
| | (precursor was second | | | | Hz, 1H), 1.63-1.57 (m, 3H), 1.34 |
| | eluting peak by SFC using | | | | (s, 3H), 0.73 (d, J = 6.0 Hz, 3H) ppm. |
| | Whelk-O1 column) | | | | |
| 3 | rel-(2R*,3S*,4S*,5R*)-3- | 504.447 | 505.3 | 3.11 | ¹H NMR (400 MHz, DMSO-d₆) δ |
| | (3,4-difluoro-2- | | | | 10.57 (s, 1H), 8.35 (d, J = 5.3 Hz, |
| | methoxyphenyl)-N-(2-(1,2- | | | | 1H), 7.84 (s, 1H), 7.52 (s, 1H), |
| | dihydroxyphenyl)-2- | | | | 7.24-7.06 (m, 2H), 5.09 (d, J = |
| | yl)pyridin-4-yl)-4,5- | | | | 10.0 Hz, 1H), 5.05 (s, 1H), 4.59 |
| | dimethyl-5- | | | | (s, 1H), 4.25 (dd, J = 10.4, 7.8 Hz, |
| | (trifluoromethyl) | | | | 1H), 3.95 (d, J = 2.3 Hz, 3H), 3.50 |
| | tetrahydrofuran-2- | | | | (d, J = 5.1 Hz, 2H), 2.85-2.70 |
| | carboxamide | | | | (m, 1H), 1.60 (s, 3H), 1.34 (s, 3H), |
| | (precursor was first eluting | | | | 0.72 (d, J = 5.6 Hz, 3H) ppm. |
| | peak by SFC using Whelk- | | | | |
| | O1 column) | | | | |
| 203 | rel-(2R*,3S*,4S*,5R*)-3- | 504.447 | 505.6 | 3.08 | ¹H NMR (500 MHz, Chloroform-d) δ |
| | (3,4-difluoro-2- | | | | 9.11 (s, 1H), 8.45 (d, J = 6.0 Hz, |
| | methoxyphenyl)-N-(2- | | | | 1H), 7.81 (s, 1H), 7.71 (s, 1H), 7.08 |
| | ((1S,2R)-1,2- | | | | (t, J = 7.2 Hz, 1H), 6.90 (q, J = 8.8 |
| | dihydroxypropyl)pyridin-4- | | | | Hz, 1H), 5.08 (d, J = 11.0 Hz, 1H), |
| | yl)-4,5-dimethyl-5- | | | | 4.68 (s, 1H), 4.16-4.10 (m, 1H), |
| | (trifluoromethyl) | | | | 4.10-4.04 (m, 1H), 4.01 (d, J = 2.8 |
| | tetrahydrofuran-2- | | | | Hz, 3H), 2.76 (t, J = 7.7 Hz, 1H), |
| | carboxamide | | | | 1.69 (s, 3H), 1.28 (d, J = 6.4 Hz, 3H), |
| | (precursor was first eluting | | | | 0.79 (d, J = 7.5 Hz, 3H) ppm. |
| | isomer by SFC using | | | | |
| | Whelk-O1 column; syn diol) | | | | |
| 204 | rel-(2R*,3S*,4S*,5R*)-3- | 504.447 | 505.6 | 3.08 | ¹H NMR (500 MHz, Chloroform-d) δ |
| | (3,4-difluoro-2- | | | | δ 9.14 (s, 1H), 8.45 (s, 1H), 7.80 (s, |
| | methoxyphenyl)-N-(2- | | | | 1H), 7.73 (s, 1H), 7.08 (d, J = 7.0 |
| | ((1R,2S)-1,2- | | | | Hz, 1H), 6.90 (s, J = 8.7 Hz, 1H), |
| | dihydroxypropyl)pyridin-4- | | | | 5.08 (d, J = 11.0 Hz, 1H), 4.70 (s, 1H), |
| | yl)-4,5-dimethyl-5- | | | | 4.17-4.10 (m, 1H), 4.07 (s, 1H), 4.01 |
| | (trifluoromethyl) | | | | (d, J = 2.7 Hz, 3H), 2.76 (t, J = 7.7 |
| | tetrahydrofuran-2- | | | | Hz, 1H), 1.69 (s, 3H), 1.27 (d, J = |
| | carboxamide | | | | 6.3 Hz, 3H), 0.79 (d, J = 7.4 Hz, 3H) |
| | (precursor was second | | | | ppm. |
| | eluting isomer by SFC | | | | |
| | using Whelk-O1 column; | | | | |
| | syn diol) | | | | |
| 205 | rel-(2R*,3S*,4S*,5R*)-3- | 504.447 | 505.6 | 3.1 | ¹H NMR (500 MHz, Chloroform-d) δ |
| | (3,4-difluoro-2- | | | | 8.86 (s, 1H), 8.50 (s, 1H), 8.42 (s, |
| | methoxyphenyl)-N-(2-(1,2- | | | | 1H), 7.06 (d, J = 10.7 Hz, 1H), 6.92 |
| | dihydroxyethyl)-5- | | | | (q, J = 8.6 Hz, 1H), 5.08 (d, J = 10.8 |
| | methylpyridin-4-yl)-4,5- | | | | Hz, 1H), 4.91 (s, 1H), 4.09 (s, 1H), |
| | dimethyl-5- | | | | 4.02 (d, J = 2.8 Hz, 3H), 3.80 (s, 1H), |
| | (trifluoromethyl) | | | | 2.78 (s, 1H), 2.35 (s, 3H), 1.69 (s, |
| | tetrahydrofuran-2- | | | | 3H), 1.26 (s, 1H), 0.80 (d, J = 7.4 |
| | carboxamide | | | | Hz, 3H) ppm. |
| | (precursor was first eluting | | | | |
| | isomer by SFC using Lux | | | | |
| | i-Cellulose column) | | | | |
| 206 | rel-(2R*,3S*,4S*,5R*)-3- | 491.408 | 492.6 | 2.92 | ¹H NMR (500 MHz, DMSO-d₆) δ |
| | (3,4-difluoro-2- | | | | 10.76 (s, 1H), 9.28 (d, J = 2.6 Hz, |
| | methoxyphenyl)-N-(6-(1,2- | | | | 1H), 8.09 (d, J = 2.5 Hz, 1H), |
| | dihydroxyethyl)pyridazin- | | | | 7.21-7.10 (m, 2H), 5.15 (d, J = 10.1 |
| | 4-yl)-4,5-dimethyl-5- | | | | Hz, 1H), 4.79 (dd, J = 6.2, 4.4 Hz, |
| | (trifluoromethyl) | | | | 1H), 4.26 (dd, J = 10.2, 7.7 Hz, 1H), |
| | tetrahydrofuran-2- | | | | 3.95 (d, J = 2.1 Hz, 3H), 3.69 (dd, J = |
| | carboxamide | | | | 11.2, 4.4 Hz, 1H), 3.55 (dd, J = 11.1, |
| | (precursor was first eluting | | | | 6.2 Hz, 1H), 2.78 (p, J = 7.5 Hz, 1H), |
| | isomer by SCF using Lux | | | | 1.61 (s, 3H), 0.76-0.68 (m, 3H) ppm. |
| | i-Cellulose column) | | | | |
| 207 | rel-(2R*,3S*,4S*,5R*)-3- | 491.408 | 492.6 | 2.92 | ¹H NMR (500 MHz, DMSO-d₆) δ |
| | (3,4-difluoro-2- | | | | 10.75 (s, 1H), 9.30 (d, J = 2.5 Hz, |
| | methoxyphenyl)-N-(6-(1,2- | | | | 1H), 8.09 (d, J = 2.5 Hz, 1H), |
| | dihydroxyethyl)pyridazin- | | | | 7.24-7.14 (m, 2H), 5.67 (d, J = 4.8 Hz, |
| | 4-yl)-4,5-dimethyl-5- | | | | 1H), 5.16 (d, J = 10.2 Hz, 1H), |
| | (trifluoromethyl) | | | | 4.84-4.75 (m, 2H), 4.28 (dd, J = |
| | tetrahydrofuran-2- | | | | 10.1, 7.7 Hz, 1H), 3.98 (d, J = 2.1 |
| | carboxamide | | | | Hz, 3H), 3.71 (ddd, J = 10.6, 6.0, 4.4 |
| | (precursor was second eluting | | | | Hz, 1H), 3.57 (dt, J = 11.4, 6.1 Hz, |
| | isomer by SFC using Lux | | | | 1H), 2.80 (p, J = 7.5 Hz, 1H), 1.64 |
| | i-Cellulose column) | | | | (s, 3H), 0.76 (d, J = 7.4 Hz, 3H). |

The following compounds were made using the method described in Example 7, except that different coupling partners were used in the amide coupling step 8. Diastereomers generated in step 8 were separated by chiral SFC and deprotection using General Method C was carried out as the final step. In the Table below, “MS r.t.” stands for Mass Spec retention time.

| Cmpd | | LC/MS | Found | MS | NMR (shifts |
|---|---|---|---|---|---|
| No. | Compound Name | (m/z calc.) | M + 1 | r.t. | in ppm) |
| 208 | rel-(2R*,3S*,4S*,5R*)-3- | 504.447 | 505.6 | 3.09 | |
| | (3,4-difluoro-2- | | | | |
| | methoxyphenyl)-N-(2- | | | | |
| | ((1S,2S)-1,2- | | | | |
| | dihydroxypropyl)pyridin- | | | | |
| | 4-yl)-4,5-dimethyl-5- | | | | |
| | (trifluoromethyl) | | | | |
| | tetrahydrofuran-2- | | | | |
| | carboxamide | | | | |
| | (precursor was first | | | | |
| | eluting isomer by SFC | | | | |
| | using Lux i-Cellulose-5 | | | | |
| | column) | | | | |

The following compound was made using the method described in Example 7, except that 2-(2,2-dimethyl-1,3-dioxolan-4-yl)pyridin-4-amine (second eluting isomer by SFC using a Chiralpak ID column) was used in the amide coupling step 8 and General Method A at 0° C., then General Method B were used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 210 | rel-4-((2R*,3S*,4S*,5R*)-3- | 506.42 | 507.6 | 2.85 | |
| | (3,4-difluoro-2- | | | | |
| | methoxyphenyl)-4,5- | | | | |
| | dimethyl-5- | | | | |
| | (trifluoromethyl)tetrahydrofu | | | | |
| | ran-2-carboxamido)-2-(1,2- | | | | |
| | dihydroxyethyl)pyridine 1- | | | | |
| | oxide | | | | |
| 209 | rel-(2R*,3S*,4S*,5R*)-3- | 504.447 | 506.6 | 3.09 | ¹H NMR (500 MHz, |
| | (3,4-difluoro-2- | | | | DMSO-d₆) δ 10.55 (s, 1H), |
| | methoxyphenyl)-N-(2- | | | | 8.33 (d, J = 5.5 Hz, 1H), |
| | ((1R,2R)-1,2- | | | | 7.68 (d, J = 2.1 Hz, 1H), |
| | dihydroxypropyl)pyridin- | | | | 7.52 (dd, J = 5.6, 2.1 Hz, |
| | 4-yl)-4,5-dimethyl-5- | | | | 1H), 7.23-7.07 (m, 2H), |
| | (trifluoromethyl) | | | | 5.27 (d, J = 4.8 Hz, 1H), |
| | tetrahydrofuran-2- | | | | 5.07 (d, J = 10.3 Hz, 1H), |
| | carboxamide | | | | 4.64 (d, J = 5.2 Hz, 1H), |
| | (precursor was second | | | | 4.41 (t, J = 4.7 Hz, 1H), |
| | eluting isomer by SFC | | | | 4.42 (dd, J = 10.3, 7.6 Hz, |
| | using Lux i-Cellulose-5 | | | | 1H), 3.95 (d, J = 2.0 Hz, |
| | column) | | | | 3H), 3.90-3.82 (m, 1H), |
| | | | | | 2.76 (p, J = 7.5 Hz, 1H), |
| | | | | | 1.59 (s, 3H), 0.91 (d, J = |
| | | | | | 6.3 Hz, 3H), 0.78-0.65 |
| | | | | | (m, 3H) ppm. |

The following compound was made using the method described in Example 7, except that 2-methyl-5-methylsulfanyl-pyrazol-3-amine was used in the amide coupling step 8 and General Method D was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 211 | (2R,3S,4S,5R)-3-(3,4- | 511.463 | 512 | 3.26 | ¹H NMR (500 MHz, DMSO- |
| | difluoro-2- | | | | d₆) δ 10.46 (s, 1H), 7.35-7.06 |
| | methoxyphenyl)-4,5- | | | | (m, 2H), 6.73 (s, 1H), 5.19 (d, |
| | dimethyl-N-(1-methyl- | | | | J = 10.2 Hz, 1H), 4.22 (dd, J = |
| | 3-(methylsulfonyl)- | | | | 10.3, 7.6 Hz, 1H), 3.95 (d, J = |
| | 1H-pyrazol-5-yl)-5- | | | | 2.0 Hz, 3H), 3.71 (s, 3H), 3.18 |
| | (trifluoromethyl) | | | | (s, 3H), 2.77 (p, J = 7.5 Hz, |
| | tetrahydrofuran-2- | | | | 1H), 1.62 (s, 3H), 0.80-0.65 |
| | carboxamide | | | | (m, 3H) ppm. |

The following compounds were made using the method described in Example 7, except that different coupling partners were used in the amide coupling step 8 and General Method I was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M+1 | r.t. | NMR (shifts in ppm) |
| 212 | (2R,3S,4S,5R)-N-(1H- | 469.405 | 470 | 3.15 | ¹H NMR (500 MHz, DMSO- |
| | benzo[d]imidazol-6- | | | | d₆) δ 12.35 (s, 1H), 10.23 (s, |
| | yl)-3-(3,4-difluoro-2- | | | | 1H), 8.14 (s, 1H), 8.00 (s, 1H), |
| | methoxyphenyl)-4,5- | | | | 7.51 (s, 1H), 7.26 (s, 1H), |
| | dimethyl-5- | | | | 7.22-7.10 (m, 2H), 5.08 (d, J = |
| | (trifluoromethyl) | | | | 10.4 Hz, 1H), 4.27 (dd, J = |
| | tetrahydrofuran-2- | | | | 10.4, 7.6 Hz, 1H), 3.96 (d, J = |
| | carboxamide | | | | 2.0 Hz, 3H), 2.76 (p, J = 7.5 |
| | | | | | Hz, 1H), 1.61 (s, 3H), 0.74 (d, |
| | | | | | 3H) ppm. |
| 213 | (2R,3S,4S,5R)-N-(6-(3- | 501.446 | 502.6 | 3.13 | ¹H NMR (500 MHz, DMSO- |
| | aminooxetan-3- | | | | d₆) δ 10.43 (s, 1H), 8.75 (d, J = |
| | yl)pyridin-3-y1)-3- | | | | 2.6 Hz, 1H), 8.05 (dd, J = 8.6, |
| | (3,4-difluoro-2- | | | | 2.6 Hz, 1H), 7.60 (d, J = 8.6 |
| | methoxyphenyl)-4,5- | | | | Hz, 1H), 7.20 - 7.13 (m, 2H), |
| | dimethyl-5- | | | | 5.10 (d, J = 10.3 Hz, 1H), 4.83 |
| | (trifluoromethyl) | | | | (dd, J = 5.5, 1.6 Hz, 2H), 4.53 |
| | tetrahydrofuran-2- | | | | (d, J = 5.5 Hz, 2H), 4.25 (dd, |
| | carboxamide | | | | J = 10.3, 7.6 Hz, 1H), 3.95 (d, |
| | | | | | J = 1.9 Hz, 3H), 2.77 (dq, J = |
| | | | | | 7.5, 7.5 Hz, 1H), 2.67-2.62 |
| | | | | | (m, 2H), 1.61 (s, 3H), 0.73 (d, |
| | | | | | J = 6.5 Hz, 3H) ppm. |
| 214 | (2R,3S,4S,5R)-3-(3,4- | 529.5 | 530.22 | 3.23 | ¹H NMR (500 MHz, DMSO- |
| | difluoro-2- | | | | d₆) δ 10.50 (s, 1H), 8.35 (d, J = |
| | methoxyphenyl)-4,5- | | | | 5.6 Hz, 1H), 7.50 (d, J = 2.0 |
| | dimethyl-N-(2-(((S)- | | | | Hz, 1H), 7.44 (dd, J = 5.6, 2.1 |
| | morpholin-3- | | | | Hz, 1H), 7.23-7.13 (m, 1H), |
| | yl)methyl)pyridin-4- | | | | 7.17 - 7.09 (m, 1H), 5.10 (d, |
| | yl)-5- | | | | J = 10.3 Hz, 1H), 4.25 (dd, J = |
| | (trifluoromethyl) | | | | 10.3, 7.6 Hz, 1H), 3.96 (d, J = |
| | tetrahydrofuran-2- | | | | 2.2 Hz, 3H), 3.61 (dq, J = 10.4, |
| | carboxamide | | | | 2.8 Hz, 2H), 3.38-3.27 (m, |
| | | | | | 1H), 3.08 (dd, J = 10.6, 9.5 Hz, |
| | | | | | 1H), 2.99 (dtd, J = 9.4, 6.5, 2.7 |
| | | | | | Hz, 1H), 2.78 (q, J = 7.5 Hz, |
| | | | | | 1H), 2.77-2.64 (m, 2H), 2.59 |
| | | | | | (d, J = 6.6 Hz, 2H), 2.33 (s, |
| | | | | | 1H), 1.60 (s, 3H), 0.73 (dt, |
| | | | | | J = 7.5, 2.4 Hz, 3H) ppm. |
| 215 | (2R,3S,4S,5R)-3-(3,4- | 527.527 | 528.27 | 3.73 | ¹H NMR (500 MHz, DMSO- |
| | difluoro-2- | | | | Methanol-d₄) δ 9.21 (dt, J = |
| | methoxyphenyl)-4,5- | | | | 2.5, 0.8 Hz, 1H), 8.55-8.48 |
| | dimethyl-N-(6- | | | | (m, 1H), 7.81 (d, J = 8.7 Hz, |
| | (piperidin-4- | | | | 1H), 7.17 (ddd, J = 8.2, 5.6, |
| | ylmethyl)pyridin-3- | | | | 2.2 Hz, 1H), 6.99 (ddd, J = 9.9, |
| | yl)-5- | | | | 8.9, 7.6 Hz, 1H), 5.16 (d, J = |
| | (trifluoromethyl) | | | | 10.5 Hz, 1H), 4.33 (dd, J = |
| | tetrahydrofuran-2- | | | | 10.5, 8.0 Hz, 1H), 4.01 (d, J = |
| | carboxamide | | | | 2.3 Hz, 3H), 3.74 (hept, J = 6.6 |
| | | | | | Hz, 1H), 3.40 (dt, J = 13.2, 3.3 |
| | | | | | Hz, 2H), 3.24 (q, J = 7.4 Hz, |
| | | | | | 1H), 3.03-2.95 (m, 3H), |
| | | | | | 2.99-2.91 (m, 1H), 2.80 (q, J = |
| | | | | | 7.6 Hz, 1H), 2.14 (dqd, J = |
| | | | | | 11.2, 7.5, 3.6 Hz, 1H), 1.92-1.82 |
| | | | | | (m, 2H), 1.68 (t, J = 1.2 Hz, |
| | | | | | 3H), 1.64-1.49 (m, 2H), |
| | | | | | 0.87-0.79 (m, 3H) ppm. |
| 216 | (2R,3S,4S,5R)-3-(3,4- | 529.5 | 530.2 | 3.23 | ¹H NMR (500 MHz, DMSO- |
| | difluoro-2- | | | | d₆) δ 10.48 (s, 1H), 8.38-8.32 |
| | methoxyphenyl)-4,5- | | | | (m, 1H), 7.49 (t, J = 1.3 Hz, |
| | dimethyl-N-(2-(((R)- | | | | 1H), 7.44 (dd, J = 5.6, 2.1 Hz, |
| | morpholin-3- | | | | 1H), 7.23-7.08 (m, 2H), 5.09 |
| | yl)methyl)pyridin-4- | | | | (d, J = 10.3 Hz, 1H), 4.25 (dd, |
| | y1)-5- | | | | J = 10.3, 7.6 Hz, 1H), 3.95 (d, |
| | (trifluoromethyl)tetrah | | | | J = 2.2 Hz, 3H), 3.60 (dt, J = |
| | ydrofuran-2- | | | | 10.1, 3.7 Hz, 2H), 3.37-3.27 |
| | carboxamide | | | | (m, 1H), 3.12-2.93 (m, 2H), |
| | | | | | 2.83-2.56 (m, 4H), 1.60 (s, |
| | | | | | 3H), 0.73 (d, J = 7.4 Hz, 3H) |
| | | | | | ppm. |

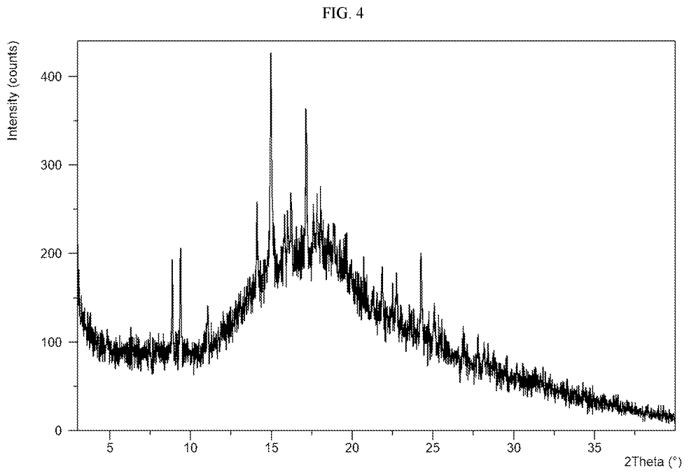

Compound 213 was analyzed by X-ray powder diffraction and determined to be partially crystalline (see FIG. 4).

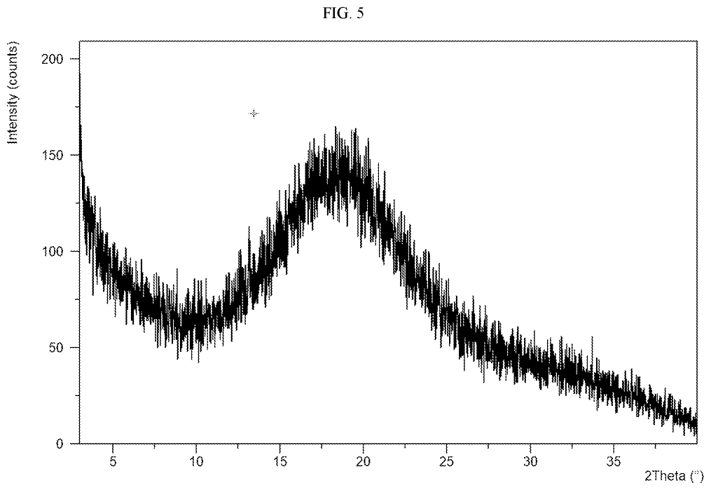

Compound 215 was analyzed by X-ray powder diffraction and determined to be amorphous (see FIG. 5).

The following compounds were made using the method described in Example 7, except that tert-butyl N-[1-(6-aminopyrimidin-4-yl)-2-methoxy-ethyl]carbamate was used in the amide coupling step 8. SFC was used to separate diastereomeric products generated in step 8 and General Method I was used to deprotect the separated isomers as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 217 | rel- | 504.45 | 505.6 | 3.25 | ¹H NMR (500 MHz, DMSO- |
| | (2R*,3S*,4S*,5R*)-N- | | | | d₆) δ 11.06 (s, 1H), 8.86 (s, |
| | (6-(1-amino-2- | | | | 1H), 8.18 (s, 1H), 7.22-7.16 |
| | methoxyethyl) | | | | (m, 2H), 5.26 (d, J = 10.5 Hz, |
| | pyrimidin-4-y1)-3- | | | | 1H), 4.26 (dd, J = 10.5, 7.5 Hz, |
| | (3,4-difluoro-2- | | | | 1H), 4.11 (t, J = 5.8 Hz, 1H), |
| | methoxyphenyl)-4,5- | | | | 3.95 (d, J = 2.0 Hz, 3H), 3.56 |
| | dimethyl-5- | | | | (dd, J = 9.6, 5.2 Hz, 1H), 3.48 |
| | (trifluoromethyl) | | | | (dd, J = 9.6, 6.4 Hz, 1H), 3.22 |
| | tetrahydrofuran-2- | | | | (s, 3H), 2.77 (dq, J = 7.5, 7.5 |
| | carboxamide | | | | Hz, 1H), 1.60 (s, 3H), 0.71 (d, |
| | (precursor was first | | | | J = 6.5 Hz, 3H) ppm. |
| | eluting isomer by SFC | | | | |
| | using Chiralpak IG | | | | |
| | column) | | | | |
| 218 | rel- | 504.45 | 505.6 | 3.25 | ¹H NMR (500 MHz, DMSO- |
| | (2R*,3S*,4S*,5R*)-N- | | | | d₆) δ 11.01 (s, 1H), 8.81 (s, |
| | (6-(1-amino-2- | | | | 1H), 8.16 (s, 1H), 7.22-7.15 |
| | methoxyethyl) | | | | (m, 2H), 5.25 (d, J = 10.5 Hz, |
| | pyrimidin-4-yl)-3- | | | | 1H), 4.25 (dd, J = 10.5, 7.5 Hz, |
| | (3,4-difluoro-2- | | | | 1H), 3.98 - 3.95 (m, 1H), 3.95 |
| | methoxyphenyl)-4,5- | | | | (d, J = 2.2 Hz, 3H), 3.52 (dd, |
| | dimethyl-5- | | | | J = 9.3, 5.4 Hz, 1H), 3.42 (dd, |
| | (trifluoromethyl) | | | | J = 9.3, 6.4 Hz, 1H), 3.20 (s, |
| | tetrahydrofuran-2- | | | | 3H), 2.77 (dq, J = 7.5, 7.5 Hz, |
| | carboxamide | | | | 1H), 1.95 (s, 2H), 1.60 (s, 3H), |
| | (precursor was second | | | | 0.71 (d, J = 6.4 Hz, 3H) ppm. |
| | eluting isomer by SFC | | | | |
| | using Chiralpak IG | | | | |
| | column) | | | | |

The following compound was made using the method described in Example 7, except that 6-[1-[tert-butyl(dimethyl)silyl]oxy-1-methyl-ethyl]pyridazin-4-amine was used in the amide coupling step 8 and General Method J was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 219 | (2R,3S,4S,5R)-3-(3,4- | 489.436 | 490.2 | 3.26 | ¹H NMR (500 MHz, DMSO- |
| | difluoro-2- | | | | d₆) δ 10.66 (s, 1H), 9.20 (d, J = |
| | methoxyphenyl)-N-(6- | | | | 2.5 Hz, 1H), 8.19 (d, J = 2.5 |
| | (2-hydroxypropan-2- | | | | Hz, 1H), 7.16-7.04 (m, 2H), |
| | yl)pyridazin-4-yl)-4,5- | | | | 5.37 (s, 1H), 5.09 (d, J = 10.2 |
| | dimethyl-5- | | | | Hz, 1H), 4.21 (dd, J = 10.2, 7.7 |
| | (trifluoromethyl) | | | | Hz, 1H), 3.90 (d, J = 2.1 Hz, |
| | tetrahydrofuran-2- | | | | 3H), 2.73 (t, J = 7.5 Hz, 1H), |
| | carboxamide | | | | 1.56 (s, 3H), 1.44 (s, 6H), |
| | | | | | 0.74-0.62 (m, 3H) ppm. |

The following compounds were made using the method described in Example 7, except that different amine coupling partners were used in the amide coupling step 8. SFC was used to separate diastereomeric products generated in step 8 and General Method J was used as the final step on the separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 220 | rel- | 516.458 | 517.7 | 3.23 | ¹H NMR (500 MHz, DMSO- |
| | (2R*,3S*,4S*,5R*)-3- | | | | d₆) δ 10.61 (s, 1H), 8.40 (d, J = |
| | (3,4-difluoro-2- | | | | 5.5 Hz, 1H), 7.97 (d, J = 2.1 |
| | methoxyphenyl)-N-(2- | | | | Hz, 1H), 7.54 (dd, J = 5.5, 2.1 |
| | (3-hydroxy- | | | | Hz, 1H), 7.26 - 7.06 (m, 2H), |
| | tetrahydrofuran- | | | | 5.60 (s, 1H), 5.10 (d, J = 10.3 |
| | 3-yl)pyridin-4-yl)- | | | | Hz, 1H), 4.26 (dd, J = 10.4, 7.7 |
| | 4,5-dimethyl-5- | | | | Hz, 1H), 4.04-3.93 (m, 6H), |
| | (trifluoromethyl) | | | | 3.89 (d, J = 8.7 Hz, 1H), 3.74 |
| | tetrahydrofuran-2- | | | | (dd, J = 8.7, 1.0 Hz, 1H), 2.78 |
| | carboxamide | | | | (p, J = 7.4 Hz, 1H), 2.07-1.95 |
| | (first eluting isomer by | | | | (m, 1H), 1.61 (s, 3H), |
| | SFC using Lux | | | | 0.78-0.66 (m, 3H) ppm. |
| | Cellulose-2 column) | | | | |
| 221 | rel- | 516.458 | 517.7 | 3.23 | ¹H NMR (500 MHz, DMSO- |
| | (2R*,3S*,4S*,5R*)-3- | | | | d₆) δ 10.60 (s, 1H), 8.40 (d, J = |
| | (3,4-difluoro-2- | | | | 5.6 Hz, 1H), 7.98 (d, J = 2.0 |
| | methoxyphenyl)-N-(2- | | | | Hz, 1H), 7.53 (dd, J = 5.5, 2.1 |
| | (3-hydroxy- | | | | Hz, 1H), 7.22-7.10 (m, 2H), |
| | tetrahydrofuran- | | | | 5.59 (s, 1H), 5.09 (d, J = 10.2 |
| | 3-yl)pyridin-4-yl)- | | | | Hz, 1H), 4.25 (dd, J = 10.3, 7.6 |
| | 4,5-dimethyl-5- | | | | Hz, 1H), 4.06-3.92 (m, 4H), |
| | (trifluoromethyl) | | | | 3.90 (d, J = 8.7 Hz, 1H), 3.73 |
| | tetrahydrofuran-2- | | | | (dd, J = 8.5, 1.0 Hz, 1H), 3.29 |
| | carboxamide | | | | (s, 1H), 2.78 (p, J = 7.5 Hz, |
| | (second eluting isomer | | | | 1H), 2.00 (ddt, J = 12.3, 6.8, |
| | by SFC using Lux | | | | 3.6 Hz, 1H), 1.61 (s, 3H), |
| | Cellulose-2 column) | | | | 0.76-0.65 (m, 3H) ppm. |
| 222 | rel- | 504.447 | 505.35 | 3.205 | ¹H NMR (500 MHz, DMSO- |
| | (2R*,3S*,4S*,5R*)-3- | | | | d₆) δ 10.58 (s, 1H), 8.34 (d, J = |
| | (3,4-difluoro-2- | | | | 5.5 Hz, 1H), 7.73 (d, J = 2.0 |
| | methoxyphenyl)-N-(2- | | | | Hz, 1H), 7.52 (dd, J = 5.5, 2.2 |
| | (1-hydroxy-2- | | | | Hz, 1H), 7.24-7.08 (m, 2H), |
| | methoxyethyl)pyridin- | | | | 5.52 (s, 1H), 5.08 (d, J = 10.2 |
| | 4-yl)-4,5-dimethyl-5- | | | | Hz, 1H), 4.67 (dd, J = 6.9, 3.6 |
| | (trifluoromethyl) | | | | Hz, 1H), 4.25 (dd, J = 10.3, 7.6 |
| | tetrahydrofuran-2- | | | | Hz, 1H), 3.96 (d, J = 2.1 Hz, |
| | carboxamide | | | | 3H), 3.60 (dd, J = 10.1, 3.6 Hz, |
| | (first eluting isomer by | | | | 1H), 3.43 (dd, J = 10.1, 7.0 Hz, |
| | SFC using Whelk-O1 | | | | 1H), 3.25 (s, 3H), 2.77 (p, J = |
| | column) | | | | 7.5 Hz, 1H), 1.60 (s, 3H), |
| | | | | | 0.77-0.66 (m, 3H) ppm. |
| 223 | rel- | 504.447 | 505.35 | 3.205 | ¹H NMR (500 MHz, DMSO- |
| | (2R*,3S*,4S*,5R*)-3- | | | | d₆) δ 10.58 (s, 1H), 8.33 (d, J = |
| | (3,4-difluoro-2- | | | | 5.5 Hz, 1H), 7.72 (d, J = 2.1 |
| | methoxyphenyl)-N-(2- | | | | Hz, 1H), 7.50 (dd, J = 5.5, 2.2 |
| | (1-hydroxy-2- | | | | Hz, 1H), 7.24-7.08 (m, 2H), |
| | methoxyethyl)pyridin- | | | | 5.07 (d, J = 10.3 Hz, 1H), 4.67 |
| | 4-yl)-4,5-dimethyl-5- | | | | (dd, J = 6.8, 3.5 Hz, 1H), 4.25 |
| | (trifluoromethyl) | | | | (dd, J = 10.3, 7.6 Hz, 1H), 3.96 |
| | tetrahydrofuran-2- | | | | (d, J = 2.1 Hz, 3H), 3.59 (dd, |
| | carboxamide | | | | J = 10.1, 3.6 Hz, 1H), 3.43 (dd, |
| | (second eluting isomer | | | | J = 10.1, 7.0 Hz, 1H), 3.25 (s, |
| | by SFC using Whelk- | | | | 3H), 2.77 (p, J = 7.5 Hz, 1H), |
| | O1 column) | | | | 1.60 (s, 3H), 0.94 (t, J = 7.4 |
| | | | | | Hz, 1H), 0.78-0.63 (m, 3H) |
| | | | | | ppm. |
| 224 | rel- | 506.438 | 507.5 | 3.43 | |
| | (2R*,3S*,4S*,5R*)-3- | | | | |
| | (3,4-difluoro-2- | | | | |
| | methoxyphenyl)-N-(6- | | | | |
| | (1-fluoro-2- | | | | |
| | hydroxypropan-2- | | | | |
| | yl)pyridin-3-yl)-4,5- | | | | |
| | dimethyl-5- | | | | |
| | (trifluoromethyl) | | | | |
| | tetrahydrofuran-2- | | | | |
| | carboxamide | | | | |
| | (first eluting isomer by | | | | |
| | SFC using OD-H | | | | |
| | column) | | | | |
| 225 | rel- | 506.438 | 507.4 | 3.43 | ¹H NMR (500 MHz, DMSO- |
| | (2R*,3S*,4S*,5R*)-3- | | | | d₆) δ 10.42 (s, 1H), 8.68 (d, J = |
| | (3,4-difluoro-2- | | | | 2.5 Hz, 1H), 8.03 (dd, J = 8.6, |
| | methoxyphenyl)-N-(6- | | | | 2.5 Hz, 1H), 7.62 (d, J = 8.6 |
| | (1-fluoro-2- | | | | Hz, 1H), 7.17 (dd, J = 8.5, 4.4 |
| | hydroxypropan-2- | | | | Hz, 2H), 5.60 (s, 1H), 5.10 (d, |
| | yl)pyridin-3-yl)-4,5- | | | | J = 10.2 Hz, 1H), 4.59-4.49 |
| | dimethyl-5- | | | | (m, 1H), 4.49-4.40 (m, 1H), |
| | (trifluoromethyl) | | | | 4.24 (dd, J = 10.3, 7.6 Hz, 1H), |
| | tetrahydrofuran-2- | | | | 3.95 (d, J = 2.0 Hz, 3H), 2.77 |
| | carboxamide | | | | (p, J = 7.5 Hz, 1H), 1.61 (s, |
| | (second eluting isomer | | | | 3H), 1.38 (d, J = 2.2 Hz, 3H), |
| | by SFC using OD-H | | | | 0.74 (dd, J = 7.2, 2.4 Hz, 3H) |
| | column) | | | | ppm. |
| 226 | rel- | 506.438 | 507.3 | 3.51 | ¹H NMR (500 MHz, DMSO- |
| | (2R*,3S*,4S*,5R*)-3- | | | | d₆) δ 10.61 (s, 1H), 8.38 (d, J = |
| | (3,4-difluoro-2- | | | | 5.5 Hz, 1H), 7.91 (d, J = 2.1 |
| | methoxyphenyl)-N-(2- | | | | Hz, 1H), 7.54 (dd, J = 5.5, 2.1 |
| | (1-fluoro-2- | | | | Hz, 1H), 7.16 (pd, J = 9.2, 6.7 |
| | hydroxypropan-2- | | | | Hz, 2H), 5.63 (s, 1H), 5.09 (d, |
| | yl)pyridin-4-yl)-4,5- | | | | J = 10.3 Hz, 1H), 4.50 (ddd, J = |
| | dimethyl-5- | | | | 48.3, 27.9, 9.0 Hz, 2H), 4.25 |
| | (trifluoromethyl) | | | | (dd, J = 10.3, 7.6 Hz, 1H), 3.96 |
| | tetrahydrofuran-2- | | | | (d, J = 2.0 Hz, 3H), 2.77 (p, J = |
| | carboxamide | | | | 7.5 Hz, 1H), 1.61 (s, 4H), 1.38 |
| | (first eluting isomer by | | | | (d, J = 2.1 Hz, 3H), 0.73 (dd, |
| | SFC using Lux | | | | J = 7.4, 2.6 Hz, 3H) ppm. |
| | Cellulose-2 column) | | | | |
| 227 | rel- | 506.438 | 507.3 | 3.51 | ¹H NMR (500 MHz, DMSO- |
| | (2R*,3S*,4S*,5R*)-3- | | | | d₆) δ 10.53 (s, 1H), 8.29 (d, J = |
| | (3,4-difluoro-2- | | | | 5.5 Hz, 1H), 7.83 (d, J = 2.0 |
| | methoxyphenyl)-N-(2- | | | | Hz, 1H), 7.45 (dd, J = 5.5, 2.1 |
| | (1-fluoro-2- | | | | Hz, 1H), 7.19-6.97 (m, 2H), |
| | hydroxypropan-2- | | | | 5.55 (s, 1H), 5.00 (d, J = 10.3 |
| | yl)pyridin-4-yl)-4,5- | | | | Hz, 1H), 4.55-4.30 (m, 2H), |
| | dimethyl-5- | | | | 4.17 (dd, J = 10.3, 7.6 Hz, 1H), |
| | (trifluoromethyl) | | | | 3.88 (d, J = 2.0 Hz, 3H), 2.69 |
| | tetrahydrofuran-2- | | | | (p, J = 7.5 Hz, 1H), 1.52 (s, |
| | carboxamide | | | | 3H), 1.29 (d, J = 2.1 Hz, 3H), |
| | (second eluting isomer | | | | 0.65 (dd, J = 7.3, 2.6 Hz, 3H) |
| | by SFC using Lux | | | | ppm. |
| | Cellulose-2 column) | | | | |
| 228 | rel- | 507.426 | 508.2 | 3.48 | ¹H NMR (500 MHz, DMSO- |
| | (2R*,3S*,4S*,5R*)-3- | | | | d₆) δ 11.06 (s, 1H), 8.86 (d, J = |
| | (3,4-difluoro-2- | | | | 1.3 Hz, 1H), 8.39 (d, J = 1.3 |
| | methoxyphenyl)-N-(6- | | | | Hz, 1H), 7.20 (dd, J = 8.5, 4.9 |
| | (1-fluoro-2- | | | | Hz, 2H), 5.84 (s, 1H), 5.26 (d, |
| | hydroxypropan-2- | | | | J = 10.5 Hz, 1H), 4.64 (d, J = |
| | yl)pyrimidin-4-yl)-4,5- | | | | 9.1 Hz, 0.5H), 4.53 (dd, J = |
| | dimethyl-5- | | | | 17.1, 9.1 Hz, 1H), 4.41 (d, J = |
| | (trifluoromethyl) | | | | 9.0 Hz, 0.5H), 4.27 (dd, J = |
| | tetrahydrofuran-2- | | | | 10.4, 7.5 Hz, 1H), 3.96 (d, J = |
| | carboxamide | | | | 2.0 Hz, 3H), 2.78 (t, J = 7.5 |
| | (first eluting isomer by | | | | Hz, 1H), 1.61 (s, 3H), 1.34 (d, |
| | SFC using Whelk-O1 | | | | J = 2.3 Hz, 3H), 0.77- 0.68 |
| | column) | | | | (m, 3H) ppm. |
| 229 | rel- | 507.426 | 508.3 | 3.47 | |
| | (2R*,3S*,4S*,5R*)-3- | | | | |
| | (3,4-difluoro-2- | | | | |
| | methoxyphenyl)-N-(6- | | | | |
| | (1-fluoro-2- | | | | |
| | hydroxypropan-2- | | | | |
| | yl)pyrimidin-4-yl)-4,5- | | | | |
| | dimethyl-5- | | | | |
| | (trifluoromethyl) | | | | |
| | tetrahydrofuran-2- | | | | |
| | carboxamide | | | | |
| | (second eluting isomer | | | | |
| | by SFC using Whelk- | | | | |
| | O1 column) | | | | |

The following compounds were made using the method described in Example 7, except that tert-butyl N-[1-(4-amino-2-pyridyl)-2-[tert-butyl(dimethyl)silyl]oxy-ethyl]-N-methyl-carbamate was used in the amide coupling step 8. Purification by SFC using a Chiralpak IC column, 5 μm particle size, 25 cm×20 mm from Daicel was used to separate diastereomeric products generated in step 8 and General Methods I (using HCl in dioxane instead of TFA), General Methods J and K were used sequentially as the final steps on the separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 230 | rel- | 517.489 | 518.4 | 2.79 | ¹H NMR (500 MHz, DMSO-d₆) |
| | (2R*,3S*,4S*,5R*)-3- | | | | δ 10.59 (s, 1H), 8.41 (d, J = 5.5 |
| | (3,4-difluoro-2- | | | | Hz, 1H), 7.68 (s, 1H), 7.53 (dd, |
| | methoxyphenyl)-N-(2- | | | | J = 5.5, 2.1 Hz, 1H), 7.22-7.07 |
| | (-1-(dimethylamino)- | | | | (m, 2H), 5.09 (d, J = 10.3 Hz, |
| | 2- | | | | 1H), 4.72 (s, 1H), 4.25 (dd, J = |
| | hydroxyethyl)pyridin- | | | | 10.3, 7.6 Hz, 1H), 3.95 (d, J = |
| | 4-yl)-4,5-dimethyl-5- | | | | 2.2 Hz, 3H), 3.80 (s, 3H), 2.84- |
| | (trifluoromethyl) | | | | 2.71 (m, 1H), 2.46-2.09 (m, |
| | tetrahydrofuran-2- | | | | 6H), 1.59 (s, 3H), 0.77-0.63 |
| | carboxamide | | | | (m, 3H) ppm. |
| | (precursor was first | | | | |
| | eluting isomer by | | | | |
| | SFC) | | | | |
| 231 | rel- | 517.489 | 518 | 2.75 | ¹H NMR (500 MHz, DMSO-d₆) |
| | (2R*,3S*,4S*,5R*)-3- | | | | δ 10.52 (s, 1H), 8.37 (d, J = 5.5 |
| | (3,4-difluoro-2- | | | | Hz, 1H), 7.61 (s, 1H), 7.51 (dd, |
| | methoxyphenyl)-N-(2- | | | | J = 5.5, 2.1 Hz, 1H), 7.24-7.03 |
| | (1-(dimethylamino)-2- | | | | (m, 2H), 5.08 (d, J = 10.3 Hz, |
| | hydroxyethyl)pyridin- | | | | 1H), 4.47 (s, 1H), 4.25 (dd, J = |
| | 4-yl)-4,5-dimethyl-5- | | | | 10.3, 7.7 Hz, 1H), 3.95 (d, J = |
| | (trifluoromethyl) | | | | 2.1 Hz, 3H), 3.83-3.67 (m, |
| | tetrahydrofuran-2- | | | | 2H), 3.45-3.37 (m, 1H), 2.77 |
| | carboxamide | | | | (p, J = 7.5 Hz, 1H), 2.18 (s, 6H), |
| | (precursor was second | | | | 1.60 (s, 3H), 0.78-0.65 (m, 3H) |
| | eluting isomer by | | | | ppm. |
| | SFC) | | | | |

The following compounds were made using the method described in Example 7, except that tert-butyl N-[1-(4-amino-2-pyridyl)-2-[tert-butyl(dimethyl)silyl]oxy-ethyl]-N-methyl-carbamate was used as the coupling partner in the amide coupling step 8. Purification by SFC using a Chiralpak IC column, Sum particle size, 25 cm×20 mm from Daicel was used to separate diastereomeric products generated in step 8 and global deprotection was carried out using 4 M HCl in as the final step on the separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 232 | rel- | 503.462 | 504.7 | 3.08 | ¹H NMR (500 MHz, DMSO- |
| | (2R*,3S*,4S*,5R*)-3- | | | | d₆) δ 10.55 (s, 1H), 8.37 (d, J = |
| | (3,4-difluoro-2- | | | | 5.5 Hz, 1H), 7.67 (d, J = 2.1 |
| | methoxyphenyl)-N-(2- | | | | Hz, 1H), 7.50 (dd, J = 5.6, 2.1 |
| | (2-hydroxy-1- | | | | Hz, 1H), 7.21-7.09 (m, 2H), |
| | (methylamino)ethyl) | | | | 5.09 (d, J = 10.3 Hz, 1H), 4.81 |
| | pyridin-4-yl)-4,5- | | | | (s, 1H), 4.25 (dd, J = 10.3, 7.6 |
| | dimethyl-5- | | | | Hz, 1H), 3.96 (d, J = 2.1 Hz, |
| | (trifluoromethyl) | | | | 3H), 3.58 (ddt, J = 19.8, 9.7, |
| | tetrahydrofuran-2- | | | | 4.8 Hz, 2H), 3.42-3.35 (m, |
| | carboxamide | | | | 1H), 2.77 (p, J = 7.5 Hz, 1H), |
| | (precursor was second | | | | 2.21 (s, 3H), 1.60 (s, 3H), 0.76- |
| | eluting isomer by | | | | 0.67 (m, 3H) ppm. |
| | SFC) | | | | |
| 233 | rel- | 503.462 | 504 | 3.09 | ¹H NMR (500 MHz, DMSO- |
| | (2R*,3S*,4S*,5R*)-3- | | | | d₆) δ 10.55 (s, 1H), 8.36 (d, J = |
| | (3,4-difluoro-2- | | | | 5.6 Hz, 1H), 7.64 (d, J = 2.1 |
| | methoxyphenyl)-N-(2- | | | | Hz, 1H), 7.52 (dd, J = 5.6, 2.1 |
| | (2-hydroxy-1- | | | | Hz, 1H), 7.21-7.11 (m, 2H), |
| | (methylamino)ethyl) | | | | 5.09 (d, J = 10.3 Hz, 1H), 4.77 |
| | pyridin-4-yl)-4,5- | | | | (t, J = 5.5 Hz, 1H), 4.26 (dd, J = |
| | dimethyl-5- | | | | 10.3, 7.6 Hz, 1H), 3.96 (d, J = |
| | (trifluoromethyl) | | | | 2.1 Hz, 3H), 3.57-3.53 (m, |
| | tetrahydrofuran-2- | | | | 2H), 3.37-3.33 (m, 1H), 2.78 |
| | carboxamide | | | | (p, J = 7.5 Hz, 1H), 2.19 (s, |
| | (precursor was first | | | | 3H), 2.10 (s, 1H), 1.60 (s, 3H), |
| | eluting isomer by | | | | 0.74-0.72 (m, 3H) ppm. |
| | SFC) | | | | |

The following compound was made using the method described in Example 7, except that ethyl 6-aminoimidazo [1,2-a]pyridine-2-carboxylate was used in the amide coupling step 8 and K₂CO₃ was used in place of triethylamine. General Method L was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 234 | 6-((2R,3S,4S,5R)-3- | 512.429 | 514 | 2.97 | ¹H NMR (400 MHz, DMSO- |
| | (3,4-difluoro-2- | | | | d₆) δ 10.40 (s, 1H), 9.25 (dd, J = |
| | methoxyphenyl)-4,5- | | | | 2.1, 1.0 Hz, 1H), 8.36 (d, J = |
| | dimethyl-5- | | | | 0.7 Hz, 1H), 7.61 (s, 1H), 7.58- |
| | (trifluoromethyl) | | | | 7.54 (m, 1H), 7.33 (dd, J = |
| | tetrahydrofuran-2- | | | | 9.7, 2.1 Hz, 2H), 7.22-7.12 |
| | carboxamido)imidazo[ | | | | (m, 2H), 5.11 (d, J = 10.4 Hz, |
| | 1,2-a]pyridine-2- | | | | 1H), 4.27 (dd, J = 10.3, 7.6 Hz, |
| | carboxamide | | | | 1H), 3.96 (d, J = 2.2 Hz, 3H), |
| | | | | | 2.82-2.70 (m, 1H), 1.61 (s, |
| | | | | | 3H), 0.74 (d, J = 6.8 Hz, 3H) |
| | | | | | ppm. |

The following compounds were made using the method described in Example 7, except that 5-aminopyridine-2-sulfonyl fluoride was used in the amide coupling step 8 and General Method L, using ammonia in methanol or methylamine in THF (respectively) and heating at 80° C. was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 235 | (2R,3S,4S,5R)-3-(3,4- | 509.447 | 510 | 3.26 | ¹H NMR (500 MHz, DMSO- |
| | difluoro-2- | | | | d₆) δ 10.69 (s, 1H), 8.90 (d, J = |
| | methoxyphenyl)-4,5- | | | | 2.4 Hz, 1H), 8.27 (dd, J = 8.6, |
| | dimethyl-N-(6- | | | | 2.5 Hz, 1H), 7.90 (d, J = 8.6 |
| | sulfamoylpyridin-3- | | | | Hz, 1H), 7.38 (s, 2H), 7.25- |
| | yl)-5- | | | | 7.05 (m, 2H), 5.15 (d, J = 10.2 |
| | (trifluoromethyl) | | | | Hz, 1H), 4.27 (dd, J = 10.2, 7.7 |
| | tetrahydrofuran-2- | | | | Hz, 1H), 3.96 (d, J = 2.0 Hz, |
| | carboxamide | | | | 3H), 2.79 (p, J = 7.5 Hz, 1H), |
| | | | | | 1.62 (s, 3H), 0.82-0.60 (m, |
| | | | | | 3H) ppm. |
| 236 | (2R,3S,4S,5R)-3-(3,4- | 523.473 | 524 | 3.4 | ¹H NMR (500 MHz, DMSO- |
| | difluoro-2- | | | | d₆) δ 10.74 (s, 1H), 8.91 (d, J = |
| | methoxyphenyl)-4,5- | | | | 2.4 Hz, 1H), 8.31 (dd, J = 8.6, |
| | dimethyl-N-(6-(N- | | | | 2.5 Hz, 1H), 7.89 (d, J = 8.6 |
| | methylsulfamoyl) | | | | Hz, 1H), 7.60 (s, 1H), 7.24- |
| | pyridin-3-yl)-5- | | | | 7.04 (m, 2H), 5.14 (d, J = 10.2 |
| | (trifluoromethyl) | | | | Hz, 1H), 4.27 (dd, J = 10.2, 7.7 |
| | ydrofuran-2- | | | | Hz, 1H), 3.96 (d, J = 2.0 Hz, |
| | tetrahcarboxamide | | | | 3H), 2.78 (p, J = 7.5 Hz, 1H), |
| | | | | | 2.49 (s, 3H), 1.62 (s, 3H), 0.93- |
| | | | | | 0.61 (m, 3H) ppm. |

The following compounds were made using the method described in Example 7, except that 2-[2-[tert-butyl(diphenyl)silyl]oxyethyl]pyrimidin-5-amine was used in the amide coupling step 8 and General Method M was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 237 | (2R,3S,4S,5R)-3-(3,4- | 475.409 | 476 | 3.1 | ¹H NMR (400 MHz, DMSO- |
| | difluoro-2- | | | | d₆) δ 10.43 (s, 1H), 8.91 (s, |
| | methoxyphenyl)-N-(2- | | | | 2H), 7.24-7.07 (m, 2H), 5.12 |
| | (2- | | | | (d, J = 10.2 Hz, 1H), 4.57 (t, J = |
| | hydroxyethyl)pyrimidin- | | | | 5.4 Hz, 1H), 4.23 (dd, J = |
| | 5-yl)-4,5-dimethyl- | | | | 10.2, 7.7 Hz, 1H), 3.94 (d, J = |
| | 5- | | | | 2.2 Hz, 3H), 3.80 (td, J = 6.8, |
| | (trifluoromethyl) | | | | 5.4 Hz, 2H), 2.97 (t, J = 6.8 |
| | tetrahydrofuran-2- | | | | Hz, 2H), 2.76 (p, J = 7.6 Hz, |
| | carboxamide | | | | 1H), 1.61 (s, 3H), 0.73 (d, J = |
| | | | | | 7.4 Hz, 3H) ppm. |
| 238 | (2R*,3S*,4S*,5R*)-3- | 533.445 | 534 | 3.31 | ¹H NMR (500 MHz, DMSO- |
| | (3,4-difluoro-2- | | | | d₆) δ 10.99 (d, J = 10.3 Hz, |
| | methoxyphenyl)-N-(6- | | | | 1H), 8.53 (d, J = 0.9 Hz, 1H), |
| | ((cis-4- | | | | 7.43 (dd, J = 1.8, 1.0 Hz, 1H), |
| | hydroxytetrahydrofuran- | | | | 7.26-7.11 (m, 2H), 5.38 (tt, J = |
| | 3-yl)oxy)pyrimidin- | | | | 5.3, 4.0 Hz, 1H), 5.23 (dd, J = |
| | 4-yl)-4,5-dimethyl-5- | | | | 10.4, 1.4 Hz, 1H), 5.07 (dd, J = |
| | (trifluoromethyl | | | | 24.1, 6.0 Hz, 1H), 4.36 (dtd, |
| | )tetrahydrofuran-2- | | | | J = 10.6, 5.9, 4.7 Hz, 1H), 4.24 |
| | carboxamide | | | | (ddd, J = 11.1, 7.6, 3.8 Hz, |
| | (mixture of cis isomers | | | | 1H), 4.03 (ddd, J = 9.7, 5.6, |
| | at hydroxyl-THF ring) | | | | 4.1 Hz, 1H), 3.94 (d, J = 2.1 |
| | | | | | Hz, 3H), 3.87 (ddd, J = 8.8, |
| | | | | | 5.9, 1.4 Hz, 1H), 3.72 (ddd, J = |
| | | | | | 11.0, 9.7, 4.3 Hz, 1H), 3.53 |
| | | | | | (ddd, J = 9.0, 5.8, 3.6 Hz, 1H), |
| | | | | | 2.76 (p, J = 7.5 Hz, 1H), 1.59 |
| | | | | | (s, 3H), 0.77-0.65 (m, 3H) |
| | | | | | ppm. |

The following compounds were made using the method described in Example 7, except that different coupling partners were used in the amide coupling step 8. SFC was used to separate diastereomeric products generated in step 8 and General Method M (heating at 40-50° C.) was used as the final step on the separated isomers. In the Table below, “-MS r.t.” stands for Mass Spec retention time.

| | | LC/MS | | | |
|---|---|---|---|---|---|
| Cmpd | | (m/z | Found | MS | |
| No. | Compound Name | calc.) | M + 1 | r.t. | NMR (shifts in ppm) |
| 239 | rel-(2R*,3S*,4S*,5R*)- | 532.457 | 534 | 3.35 | ¹H NMR (500 MHz, DMSO-d₆) |
| | 3-(3,4-difluoro-2- | | | | δ 10.51 (s, 1H), 8.00 (d, J = 5.8 |
| | methoxyphenyl)-N-(2- | | | | Hz, 1H), 7.19 (d, J = 1.8 Hz, |
| | (((3R,4S)-4- | | | | 1H), 7.18-7.10 (m, 3H), 5.27 |
| | hydroxytetrahydrofuran- | | | | (q, J = 5.2 Hz, 1H), 5.07 (d, J = |
| | 3-yl)oxy)pyridin-4-yl)- | | | | 10.2 Hz, 1H), 4.95 (d, J = 5.7 |
| | 4,5-dimethyl-5- | | | | Hz, 1H), 4.33 (p, J = 5.4 Hz, |
| | (trifluoromethyl) | | | | 1H), 4.24 (dd, J = 10.2, 7.7 Hz, |
| | tetrahydrofuran-2- | | | | 1H), 4.02 (dd, J = 9.3, 5.8 Hz, |
| | carboxamide | | | | 1H), 3.94 (d, J = 2.0 Hz, 3H), |
| | (precursor was first | | | | 3.87 (dd, J = 8.8, 5.7 Hz, 1H), |
| | eluting isomer by SFC | | | | 3.66 (dd, J = 9.3, 4.9 Hz, 1H), |
| | using Lux Cellulose-2 | | | | 3.55 (dd, J = 8.7, 5.3 Hz, 1H), |
| | column) | | | | 2.76 (p, J = 7.5 Hz, 1H), 1.59 (s, |
| | | | | | 3H), 0.78-0.67 (m, 3H) ppm. |
| 240 | rel-(2R*,3S*,4S*,5R*)- | 532.457 | 534 | 3.35 | ¹H NMR (500 MHz, DMSO-d₆) |
| | 3-(3,4-difluoro-2- | | | | δ 10.53 (s, 1H), 8.01 (d, J = 5.7 |
| | methoxyphenyl)-N-(2- | | | | Hz, 1H), 7.21-7.10 (m, 4H), |
| | (((3S,4R)-4- | | | | 5.27 (q, J = 5.2 Hz, 1H), 5.08 (d, |
| | hydroxytetrahydrofuran- | | | | J = 10.2 Hz, 1H), 4.97 (d, J = |
| | 3-yl)oxy)pyridin-4-yl)- | | | | 4.7 Hz, 1H), 4.34 (s, 1H), 4.25 |
| | 4,5-dimethyl-5- | | | | (dd, J = 10.1, 7.7 Hz, 1H), 4.03 |
| | (trifluoromethyl) | | | | (dd, J = 9.3, 5.8 Hz, 1H), 3.95 |
| | tetrahydrofuran-2- | | | | (d, J = 2.0 Hz, 3H), 3.88 (dd, J = |
| | carboxamide | | | | 8.7, 5.7 Hz, 1H), 3.67 (dd, J = |
| | (precursor was second | | | | 9.3, 4.9 Hz, 1H), 3.56 (dd, J = |
| | eluting isomer by SFC | | | | 8.7, 5.3 Hz, 1H), 2.77 (p, J = 7.6 |
| | using Lux Cellulose-2 | | | | Hz, 1H), 1.60 (s, 3H), 0.78- |
| | column) | | | | 0.69 (m, 3H) ppm. |
| 241 | rel-(2R*,3S*,4S*,5R*)- | 533.445 | 533 | 3.29 | ¹H NMR (500 MHz, DMSO-d₆) |
| | 3-(3,4-difluoro-2- | | | | δ 11.03-10.86 (m, 1H), 8.46 |
| | methoxyphenyl)-N-(2- | | | | (dd, J = 5.7, 0.9 Hz, 1H), 7.64 |
| | (((3R,4S)-4- | | | | (d, J = 5.5 Hz, 1H), 7.24-7.09 |
| | hydroxytetrahydrofuran- | | | | (m, 2H), 5.29 (dq, J = 5.7, 4.8 |
| | 3-yl)oxy)pyrimidin-4- | | | | Hz, 1H), 5.22 (dd, J = 10.4, 2.4 |
| | yl)-4,5-dimethyl-5- | | | | Hz, 1H), 5.08 (dd, J = 5.5, 4.4 |
| | (trifluoromethyl) | | | | Hz, 1H), 4.41 (p, J = 5.5 Hz, |
| | tetrahydrofuran-2- | | | | 1H), 4.24 (ddd, J = 10.5, 7.6, 1.3 |
| | carboxamide | | | | Hz, 1H), 4.07 (ddd, J = 9.4, 5.7, |
| | (precursor was second | | | | 2.0 Hz, 1H), 3.94 (d, J = 2.0 Hz, |
| | eluting isomer by SFC | | | | 3H), 3.90 (ddd, J = 8.8, 5.8, 1.4 |
| | using Whelk-O1 | | | | Hz, 1H), 3.73 (dd, J = 9.4, 4.8 |
| | column) | | | | Hz, 1H), 3.57 (ddd, J = 8.9, 5.5, |
| | | | | | 3.6 Hz, 1H), 2.77 (p, J = 7.5 Hz, |
| | | | | | 1H), 1.59 (s, 3H), 0.71 (dd, J = |
| | | | | | 7.5, 2.5 Hz, 3H) ppm. |
| 242 | rel-(2R*,3S*,4S*,5R*)- | 533.445 | 534 | 3.28 | ¹H NMR (500 MHz, DMSO-d₆) |
| | 3-(3,4-difluoro-2- | | | | δ 10.95 (d, J = 16.5 Hz, 1H), |
| | methoxyphenyl)-N-(2- | | | | 8.46 (dd, J = 5.6, 0.8 Hz, 1H), |
| | (((3S,4R)-4- | | | | 7.65 (d, J = 5.6 Hz, 1H), 7.31- |
| | hydroxytetrahydrofuran- | | | | 7.07 (m, 2H), 5.29 (dq, J = 5.8, |
| | 3-yl)oxy)pyrimidin-4- | | | | 4.8 Hz, 1H), 5.23 (dd, J = 10.4, |
| | yl)-4,5-dimethyl-5- | | | | 2.4 Hz, 1H), 5.09 (dd, J = 5.5, |
| | (trifluoromethyl) | | | | 4.4 Hz, 1H), 4.42 (p, J = 5.5 Hz, |
| | tetrahydrofuran-2- | | | | 1H), 4.25 (ddd, J = 10.5, 7.6, 1.3 |
| | carboxamide | | | | Hz, 1H), 4.07 (ddd, J = 9.4, 5.8, |
| | (precursor was first | | | | 2.0 Hz, 1H), 3.95 (d, J = 2.0 Hz, |
| | eluting isomer by SFC | | | | 3H), 3.91 (ddd, J = 8.8, 5.8, 1.4 |
| | using Whelk-O1 | | | | Hz, 1H), 3.74 (dd, J = 9.4, 4.8 |
| | column) | | | | Hz, 1H), 3.57 (ddd, J = 8.9, 5.5, |
| | | | | | 3.7 Hz, 1H), 2.78 (p, J = 7.5 Hz, |
| | | | | | 1H), 1.60 (s, 3H), 0.72 (dd, J = |
| | | | | | 7.6, 2.4 Hz, 3H) ppm. |

The following compounds were made using the method described in Example 7, except that different coupling partners were used in the amide coupling step 8 and deprotection using General Method N was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 18 | (2R,3S,4S,5R)-3-(3,4- | 461.383 | 462.6 | 3 | ¹H NMR (400 MHz, |
| | difluoro-2- | | | | Chloroform-d) δ 10.68 (s, 1H), |
| | methoxyphenyl)-N-(6- | | | | 9.53 (s, 1H), 8.57 (s, 1H), 7.12 |
| | (hydroxymethyl) | | | | (s, 1H), 6.95-6.83 (m, 1H), |
| | pyridazin-4-yl)-4,5- | | | | 5.20 (d, J = 10.4 Hz, 1H), 5.00 |
| | dimethyl-5- | | | | (d, J = 6.7 Hz, 2H), 4.25 (s, 1H), |
| | (trifluoromethyl) | | | | 4.03-3.96 (m, 3H), 2.75 (dt, J = |
| | tetrahydrofuran-2- | | | | 13.6, 6.7 Hz, 1H), 1.71 (s, 3H), |
| | carboxamide | | | | 0.79 (d, J = 7.3 Hz, 3H) ppm. |
| 243 | 3-(3,4-difluoro-2- | 474.421 | 475.6 | 3.23 | ¹H NMR (400 MHz, |
| | methoxy-phenyl)-N- | | | | Chloroform-d) δ 8.85 (s, 1H), |
| | [2-(hydroxymethyl)-5- | | | | 8.39 (s, 2H), 7.08 (t, J = 7.3 Hz, |
| | methyl-4-pyridyl]-4,5- | | | | 1H), 6.96-6.88 (m, 1H), 5.08 |
| | dimethyl-5- | | | | (d, J = 11.3 Hz, 1H), 4.75 (s, |
| | (trifluoromethyl) | | | | 2H), 4.08 (dd, J = 11.3, 7.8 Hz, |
| | tetrahydrofuran-2- | | | | 1H), 4.02 (d, J = 3.0 Hz, 3H), |
| | carboxamide | | | | 2.77 (q, J = 7.6 Hz, 1H), 2.35 (s, |
| | | | | | 3H), 1.70 (s, 3H), 0.85-0.75 |
| | | | | | (m, 3H) ppm. |
| 244 | (2R,3S,4S,5R)-3-(3,4- | 521.478 | 522.3 | 3.12 | ¹H NMR (500 MHz, DMSO-d₆) |
| | difluoro-2- | | | | δ 9.67 (s, 1H), 7.83 (s, 1H), 7.27- |
| | methoxyphenyl)-N-(1- | | | | 7.05 (m, 2H), 5.13 (d, J = 10.5 |
| | ((R)-2-hydroxy-3- | | | | Hz, 1H), 5.02 (d, J = 5.1 Hz, |
| | methoxypropyl)-3- | | | | 1H), 4.21 (dd, J = 10.5, 7.5 Hz, |
| | methyl-1H-pyrazol-4- | | | | 1H), 4.00 (q, J = 7.4 Hz, 1H), |
| | yl-4,5-dimethyl-5- | | | | 3.95 (d, J = 2.0 Hz, 3H), 3.92- |
| | (trifluoromethyl) | | | | 3.81 (m, 2H), 3.25 (s, 3H), 3.21 |
| | tetrahydrofuran-2- | | | | (d, J = 4.9 Hz, 2H), 2.74 (p, J = |
| | carboxamide | | | | 7.5 Hz, 1H), 2.06 (s, 3H), 1.58 |
| | | | | | (s, 3H), 0.81-0.68 (m, 3H) |
| | | | | | ppm. |
| 245 | (2R,3S,4S,5R)-3-(3,4- | 521.478 | 522.3 | 3.12 | ¹H NMR (500 MHz, DMSO-d₆) |
| | difluoro-2- | | | | δ 9.68 (s, 1H), 7.84 (s, 1H), 7.22- |
| | methoxyphenyl)-N-(1- | | | | 7.08 (m, 2H), 5.13 (d, J = 10.6 |
| | ((S)-2-hydroxy-3- | | | | Hz, 1H), 5.01 (d, J = 4.4 Hz, |
| | methoxypropyl)-3- | | | | 1H), 4.21 (dd, J = 10.6, 7.5 Hz, |
| | methyl-1H-pyrazol-4- | | | | 1H), 4.05-3.97 (m, 1H), 3.95 |
| | yl)-4,5-dimethyl-5- | | | | (d, J = 2.0 Hz, 3H), 3.91-3.79 |
| | (trifluoromethyl) | | | | (m, 2H), 3.25 (s, 3H), 3.21 (d, J = |
| | tetrahydrofuran-2- | | | | 4.8 Hz, 2H), 2.74 (p, J = 7.4 |
| | carboxamide | | | | Hz, 1H), 2.07 (s, 3H), 1.58 (s, |
| | | | | | 3H), 0.73 (dd, J = 7.4, 2.4 Hz, |
| | | | | | 3H) ppm. |
| 246 | (2R,3S,4S,5R)-3-(3,4- | 461.383 | 461.9 | 3.14 | ¹H NMR (500 MHz, DMSO-d₆) |
| | difluoro-2- | | | | δ 10.98 (s, 1H), 8.78 (d, J = 1.3 |
| | methoxyphenyl)-N-(6- | | | | Hz, 1H), 8.19 (d, J = 1.2 Hz, |
| | (hydroxymethyl) | | | | 1H), 7.20 (dd, J = 8.5, 4.7 Hz, |
| | pyrimidin-4-yl)-4,5- | | | | 2H), 5.59 (t, J = 5.8 Hz, 1H), |
| | dimethyl-5- | | | | 5.26 (d, J = 10.4 Hz, 1H), 4.55- |
| | (trifluoromethyl) | | | | 4.42 (m, 2H), 4.26 (dd, J = 10.4, |
| | tetrahydrofuran-2- | | | | 7.6 Hz, 1H), 3.95 (d, J = 2.0 Hz, |
| | carboxamide | | | | 3H), 2.77 (q, J = 7.5 Hz, 1H), |
| | | | | | 1.61 (s, 3H), 0.76-0.67 (m, 3H) |
| | | | | | ppm. |
| 247 | (2R,3S,4S,5R)-3-(3,4- | 490.42 | | 3.32 | ¹H NMR (500 MHz, DMSO-d₆) |
| | difluoro-2- | | | | δ 10.23 (s, 1H), 8.27 (d, J = 2.7 |
| | methoxyphenyl)-N-(5- | | | | Hz, 1H), 7.96 (dd, J = 2.6, 1.2 |
| | (hydroxymethyl)-6- | | | | Hz, 1H), 7.23-7.10 (m, 2H), |
| | methoxypyridin-3-yl)- | | | | 5.25 (t, J = 5.6 Hz, 1H), 5.06 (d, |
| | 4,5-dimethyl-5- | | | | J = 10.4 Hz, 1H), 4.42 (d, J = |
| | (trifluoromethyl) | | | | 5.4 Hz, 2H), 4.24 (dd, J = 10.4, |
| | tetrahydrofuran-2- | | | | 7.6 Hz, 1H), 3.96 (d, J = 2.0 Hz, |
| | carboxamide | | | | 3H), 3.83 (s, 3H), 2.76 (p, J = |
| | | | | | 7.5 Hz, 1H), 1.61 (s, 3H), 0.77- |
| | | | | | 0.68 (m, 3H) ppm. |

The following compounds were made using the method described in Example 7, except that chiral coupling partners were used in the amide coupling step 8. SFC was used to separate diastereomeric products generated in step 8 and deprotection using General Method N, was carried out as the final step on the separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 248 | rel- | 504.447 | 505.6 | 3.18 | ¹H NMR (500 MHz, DMSO-d₆) |
| | (2R*,3S*,4S*,5R*)-3- | | | | δ 10.57 (s, 1H), 8.39 (d, J = 5.6 |
| | (3,4-difluoro-2- | | | | Hz, 1H), 7.65 (d, J = 2.1 Hz, |
| | methoxyphenyl)-N-(2- | | | | 1H), 7.53 (dd, J = 5.6, 2.1 Hz, |
| | (2-hydroxy-1- | | | | 1H), 7.22-7.08 (m, 2H), 5.09 |
| | methoxyethyl)pyridin- | | | | (d, J = 10.3 Hz, 1H), 4.78 (t, J = |
| | 4-yl)-4,5-dimethyl-5- | | | | 5.9 Hz, 1H), 4.26 (dd, J = 10.3, |
| | (trifluoromethyl) | | | | 7.7 Hz, 1H), 4.21 (dd, J = 6.7, |
| | tetrahydrofuran-2- | | | | 3.8 Hz, 1H), 3.96 (d, J = 2.1 Hz, |
| | carboxamide | | | | 3H), 3.61 (ddt, J = 11.6, 6.1, 3.1 |
| | (precursor was first | | | | Hz, 1H), 3.51 (dt, J = 11.8, 6.3 |
| | eluting isomer by SFC | | | | Hz, 1H), 3.26 (s, 3H), 2.78 (p, J = |
| | using Lux Cellulose-2 | | | | 7.6 Hz, 1H), 1.61 (s, 3H), 0.73 |
| | column) | | | | (d, J = 7.5 Hz, 3H) ppm. |
| 249 | rel- | 504.447 | 505.6 | 3.17 | ¹H NMR (500 MHz, DMSO-d₆) |
| | (2R*,3S*,4S*,5R*)-3- | | | | δ 10.60 (s, 1H), 8.41 (d, J = 5.5 |
| | (3,4-difluoro-2- | | | | Hz, 1H), 7.65 (d, J = 2.1 Hz, |
| | methoxyphenyl)-N-(2- | | | | 1H), 7.57 (dd, J = 5.5, 2.1 Hz, |
| | (2-hydroxy-1- | | | | 1H), 7.24-7.11 (m, 2H), 5.11 |
| | methoxyethyl)pyridin- | | | | (d, J = 10.3 Hz, 1H), 4.80 (t, J = |
| | 4-yl)-4,5-dimethyl-5- | | | | 5.9 Hz, 1H), 4.28 (dd, J = 10.3, |
| | (trifluoromethyl) | | | | 7.7 Hz, 1H), 4.23 (dd, J = 6.6, |
| | tetrahydrofuran-2- | | | | 3.7 Hz, 1H), 3.98 (d, J = 2.1 Hz, |
| | carboxamide | | | | 3H), 3.63 (ddd, J = 11.5, 6.1, 3.9 |
| | (precursor was second | | | | Hz, 1H), 3.53 (dt, J = 11.5, 6.2 |
| | eluting isomer by SFC | | | | Hz, 1H), 3.28 (s, 3H), 2.80 (p, J = |
| | using Lux Cellulose-2 | | | | 7.5 Hz, 1H), 1.62 (s, 3H), 0.78- |
| | column) | | | | 0.72 (m, 3H) ppm. |
| 250 | rel- | 500.458 | 501.6 | 3.39 | |
| | (2R*,3S*,4S*,5R*)-N- | | | | |
| | (2- | | | | |
| | (cyclopropyl(hydroxy) | | | | |
| | methyl)pyridin-4-yl- | | | | |
| | 3-(3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| | (precursor was first | | | | |
| | eluting isomer by SFC | | | | |
| | using Lux Cellulose-2 | | | | |
| | column) | | | | |
| 251 | rel- | 500.458 | 501.6 | 3.39 | ¹H NMR (500 MHz, DMSO-d₆) |
| | (2R*,3S*,4S*,5R*)-N- | | | | δ 10.54 (s, 1H), 8.32 (d, J = 5.6 |
| | (2- | | | | Hz, 1H), 7.69 (d, J = 2.1 Hz, |
| | (cyclopropyl(hydroxy) | | | | 1H), 7.50 (dd, J = 5.6, 2.1 Hz, |
| | methyl)pyridin-4-yl)- | | | | 1H), 7.21-7.04 (m, 2H), 5.27 |
| | 3-(3,4-difluoro-2- | | | | (s, 1H), 5.07 (d, J = 10.2 Hz, |
| | methoxyphenyl)-4,5- | | | | 1H), 4.24 (dd, J = 10.3, 7.7 Hz, |
| | dimethyl-5- | | | | 1H), 4.10 (d, J = 6.6 Hz, 1H), |
| | (trifluoromethyl) | | | | 3.95 (d, J = 2.1 Hz, 3H), 2.76 (p, |
| | tetrahydrofuran-2- | | | | J = 7.3 Hz, 1H), 1.59 (s, 3H), |
| | carboxamide | | | | 1.14-0.99 (m, 1H), 0.79-0.63 |
| | (precursor was second | | | | (m, 3H), 0.41-0.26 (m, 4H) |
| | eluting isomer by SFC | | | | ppm. |
| | using Lux Cellulose-2 | | | | |
| | column) | | | | |
| 252 | rel- | 492.411 | 493.3 | 3.32 | ¹H NMR (500 MHz, DMSO-d₆) |
| | (2R*,3S*,4S*,5R*)-3- | | | | δ 10.61 (s, 1H, 8.38 (d, J = 5.5 |
| | (3,4-difluoro-2- | | | | Hz, 1H), 7.81 (d, J = 2.0 Hz, |
| | methoxyphenyl)-N-(2- | | | | 1H), 7.56 (dd, J = 5.5, 2.1 Hz, |
| | (2-fluoro-1- | | | | 1H), 7.25-7.06 (m, 2H), 5.92 |
| | hydroxyethyl)pyridin- | | | | (d, J = 5.0 Hz, 1H), 5.09 (d, J = |
| | 4-y1)-4,5-dimethyl-5- | | | | 10.3 Hz, 1H), 4.80 (dtd, J = |
| | (trifluoromethyl) | | | | 21.9, 5.7, 3.0 Hz, 1H), 4.66 |
| | tetrahydrofuran-2- | | | | (ddd, J = 47.7, 9.4, 3.0 Hz, 1H), |
| | carboxamide | | | | 4.50 (ddd, J = 47.9, 9.4, 6.1 Hz, |
| | (precursor was first | | | | 1H), 4.26 (dd, J = 103, 7.7 Hz), |
| | eluting isomer by SFC | | | | 3.96 (d, J = 2.0 Hz, 3H), 2.77 (p, |
| | using Whelk-O1 | | | | J = 7.5 Hz, 1H), 1.60 (s, 3H), |
| | column) | | | | 0.77-0.69 (m, 4H) ppm. |
| 253 | rel- | 492.411 | 492.1 | 3.31 | |
| | (2R*,3S*,4S*,5R*)-3- | | | | |
| | (3,4-difluoro-2- | | | | |
| | methoxyphenyl)-N-(2- | | | | |
| | (2-fluoro-1- | | | | |
| | hydroxyethyl)pyridin- | | | | |
| | 4-yl)-4,5-dimethyl-5- | | | | |
| | (trifluoromethyl) | | | | |
| | tetrahydrofuran-2- | | | | |
| | carboxamide | | | | |
| | (precursor was second | | | | |
| | eluting isomer by SFC | | | | |
| | using Whelk-O1 | | | | |
| | column) | | | | |
| 254 | rel- | 510.402 | 511.4 | 3.54 | ¹H NMR (500 MHz, DMSO-d₆) |
| | (2R*,3S*,4S*,5R*)-N- | | | | δ 10.64 (s, 1H), 8.42 (d, J = 5.5 |
| | (2-(2,2-difluoro-1- | | | | Hz, 1H), 7.83 (d, J = 2.0 Hz, |
| | hydroxyethyl)pyridin- | | | | 1H), 7.60 (dd, J = 5.6, 2.1 Hz, |
| | 4-yl)-3-(3,4-difluoro- | | | | 1H), 7.27-7.06 (m, 2H), 6.34 |
| | 2-methoxyphenyl)-4,5- | | | | (s, 1H), 6.20 (td, J = 55.1, 3.0 |
| | dimethyl-5- | | | | Hz, 1H), 5.10 (d, J = 10.3 Hz, |
| | (trifluoromethyl) | | | | 1H), 4.77 (ddd, J = 17.7, 8.2, 3.0 |
| | tetrahydrofuran-2- | | | | Hz, 1H), 4.26 (dd, J = 10.2, 7.7 |
| | carboxamide | | | | Hz, 1H), 3.96 (d, J = 2.0 Hz, |
| | (precursor was second | | | | 3H), 2.78 (t, J = 7.5 Hz, 1H), |
| | eluting isomer by SFC | | | | 1.61 (s, 3H), 0.80-0.69 (m, 3H) |
| | using Lux Cellulosew- | | | | ppm. |
| | 2 column) | | | | |
| 255 | rel- | 510.402 | 511 | 3.4 | ¹H NMR (500 MHz, DMSO-d₆) |
| | (2R*,3S*,4S*,5R*)-N- | | | | δ 10.65 (s, 1H), 8.42 (d, J = 5.5 |
| | (2-(2,2-difluoro-1- | | | | Hz, 1H), 7.84 (d, J = 2.0 Hz, |
| | hydroxyethyl)pyridin- | | | | 1H), 7.61 (dd, J = 5.6, 2.1 Hz, |
| | 4-yl)-3-(3,4-difluoro- | | | | 1H), 7.23-7.06 (m, 2H), 6.36 |
| | 2-methoxyphenyl)-4,5- | | | | (s, 1H), 6.21 (td, J = 55.1, 3.0 |
| | dimethyl-5- | | | | Hz, 1H), 5.10 (d, J = 10.3 Hz, |
| | (trifluoromethyl) | | | | 1H), 4.79 (dd, J = 17.3, 7.9 Hz, |
| | tetrahydrofuran-2- | | | | 1H), 4.26 (dd, J = 10.3, 7.7 Hz, |
| | carboxamide | | | | 1H), 3.96 (d, J = 1.9 Hz, 3H), |
| | (precursor was first | | | | 2.78 (p, J = 7.6 Hz, 1H), 1.61 (s, |
| | eluting isomer by SFC | | | | 3H), 0.84-0.65 (m, 3H) ppm. |
| | using Lux Cellulosew- | | | | |
| | 2 column) | | | | |

The following compounds were made using the method described in Example 7, except that 2-[1,3-bis[[tert-butyl(dimethyl)silyl]oxy]propyl]-5-fluoro-pyridin-4-amine was used in the amide coupling step 8. TBS deprotection using 1M HCl in THF, followed by chiral SFC separation afforded 2 isomers. The separated isomers were further TBS deprotected using condition similar to General Method J as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 256 | rel- | 522.437 | 523 | 3.18 | ¹H NMR (500 MHz, DMSO- |
| | (2R*,3S*,4S*,5R*)-3- | | | | d₆) δ 10.23 (s, 1H), 8.42 (d, J = |
| | (3,4-difluoro-2- | | | | 2.3 Hz, 1H), 8.18 (d, J = 6.5 |
| | methoxyphenyl)-N-(2- | | | | Hz, 1H), 7.27-7.09 (m, 2H), |
| | (1,3-dihydroxypropyl)- | | | | 5.44-5.25 (m, 2H), 4.71- |
| | 5-fluoropyridin-4-yl)- | | | | 4.55 (m, 1H), 4.39 (t, J = 5.2 |
| | 4,5-dimethyl-5- | | | | Hz, 1H), 4.25 (dd, J = 10.4, 7.5 |
| | (trifluoromethyl) | | | | Hz, 1H), 3.95 (d, J = 2.1 Hz, |
| | tetrahydrofuran-2- | | | | 3H), 3.58-3.38 (m, 2H), 2.83- |
| | carboxamide | | | | 2.69 (m, 1H), 1.93-1.81 (m, |
| | (precursor was first | | | | 1H), 1.72-1.51 (m, 4H), 0.80- |
| | eluting isomer by SFC | | | | 0.65 (m, 3H) ppm. |
| | using Whelk-O1 | | | | |
| | column) | | | | |
| 257 | rel- | 522.437 | 523 | 3.18 | |
| | (2R*,3S*,4S*,5R*)-3- | | | | |
| | (3,4-difluoro-2- | | | | |
| | methoxyphenyl)-N-(2- | | | | |
| | (1,3-dihydroxypropyl)- | | | | |
| | 5-fluoropyridin-4-yl)- | | | | |
| | 4,5-dimethyl-5- | | | | |
| | (trifluoromethyl) | | | | |
| | tetrahydrofuran-2- | | | | |
| | carboxamide | | | | |
| | (precursor was second | | | | |
| | eluting isomer by SFC | | | | |
| | using Whelk-O1 | | | | |
| | column) | | | | |

The following compounds were made using the method described in Example 7, except that ethyl 6-aminoimidazo[1,2-a]pyridine-2-carboxylate was used as the coupling partner in the amide coupling step 8 and K₂CO₃ in DCM was used instead of triethylamine in 2-MeTHF. Step 8 was followed by General Method 0 and the resulting acid was coupled with amines using conditions similar to that described in Example 7, step 8 as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 258 | 6-((2R,3S,4S,5R)-3- | 526.456 | 527 | 3.23 | ¹H NMR (400 MHz, DMSO- |
| | (3,4-difluoro-2- | | | | d₆) δ 10.40 (s, 1H), 9.24 (dd, J = |
| | methoxyphenyl)-4,5- | | | | 2.1, 1.0 Hz, 1H), 8.36 (d, J = |
| | dimethyl-5- | | | | 0.7 Hz, 1H), 8.27 (d, J = 4.9 |
| | (trifluoromethyl) | | | | Hz, 1H), 7.56 (dt, J = 9.7, 0.9 |
| | tetrahydrofuran-2- | | | | Hz, 1H), 7.34 (dd, J = 9.7, 2.1 |
| | carboxamido)-N- | | | | Hz, 1H), 7.25-7.02 (m, 2H), |
| | methylimidazo[1,2- | | | | 5.11 (d, J = 10.3 Hz, 1H), 4.27 |
| | a]pyridine-2- | | | | (dd, J = 10.5, 7.5 Hz, 1H), 3.96 |
| | carboxamide | | | | (d, J = 2.1 Hz, 3H), 2.77 (d, J = |
| | | | | | 4.8 Hz, 4H), 1.61 (s, 3H), 0.74 |
| | | | | | (d, J = 7.3 Hz, 3H) ppm. |
| 259 | (2R,3S,4S,5R)-3-(3,4- | 582.519 | 583 | 3.25 | ¹H NMR (400 MHz, DMSO- |
| | difluoro-2- | | | | d₆) δ 10.40 (s, 1H), 9.25 (dd, J = |
| | methoxyphenyl)-4,5- | | | | 2.1, 1.0 Hz, 1H), 8.38 (d, J = |
| | dimethyl-N-(2- | | | | 0.7 Hz, 1H), 7.59 (d, J = 9.6 |
| | (morpholine-4- | | | | Hz, 1H), 7.32 (dd, J = 9.7, 2.0 |
| | carbonyl)imidazo[1,2- | | | | Hz, 1H), 7.17 (dd, J = 9.2, 6.3 |
| | a]pyridin-6-yl)-5- | | | | Hz, 2H), 5.11 (d, J = 10.3 Hz, |
| | (trifluoromethyl) | | | | 1H), 4.27 (dd, J = 10.4, 7.6 Hz, |
| | tetrahydrofuran-2- | | | | 3H), 3.96 (d, J = 2.1 Hz, 3H), |
| | carboxamide | | | | 3.63 (s, 6H), 2.77 (t, J = 7.5 |
| | | | | | Hz, 1H), 1.61 (s, 3H), 0.74 (d, |
| | | | | | J = 7.3 Hz, 3H) ppm. |
| 260 | 6-((2R,3S,4S,5R)-3- | 556.482 | 557 | 3.13 | ¹H NMR (400 MHz, DMSO- |
| | (3,4-difluoro-2- | | | | d₆) δ 10.41 (s, 1H), 9.25 (dd, J = |
| | methoxyphenyl)-4,5- | | | | 2.0, 1.0 Hz, 1H), 8.38 (d, J = |
| | dimethyl-5- | | | | 0.7 Hz, 1H), 8.15 (t, J = 5.9 |
| | (trifluoromethyl) | | | | Hz, 1H), 7.61-7.53 (m, 1H), |
| | tetrahydrofuran-2- | | | | 7.34 (dd, J = 9.7, 2.0 Hz, 1H), |
| | carboxamido)-N-(2- | | | | 7.22-7.09 (m, 2H), 5.11 (d, J = |
| | hydroxyethyl)imidazo[ | | | | 10.4 Hz, 1H), 4.76 (t, J = 5.5 |
| | 1,2-a]pyridine-2- | | | | Hz, 1H), 4.27 (dd, J = 10.3, 7.5 |
| | carboxamide | | | | Hz, 1H), 3.96 (d, J = 2.2 Hz, |
| | | | | | 3H), 3.50 (q, J = 6.0 Hz, 2H), |
| | | | | | 3.38-3.32 (m, 2H), 2.77 (t, J = |
| | | | | | 7.5 Hz, 1H), 1.61 (s, 3H), |
| | | | | | 0.74 (d, J = 7.4 Hz, 3H) ppm. |
| 261 | 6-((2R,3S,4S,5R)-3- | 540.482 | 541 | 3.24 | ¹H NMR (400 MHz, DMSO- |
| | (3,4-difluoro-2- | | | | d₆) δ 10.40 (s, 1H), 9.27-9.17 |
| | methoxyphenyl)-4,5- | | | | (m, 1H), 8.33 (d, J = 0.7 Hz, |
| | dimethyl-5- | | | | 1H), 7.59 (d, J = 9.7 Hz, 1H), |
| | (trifluoromethyl) | | | | 7.32 (dd, J = 9.7, 2.1 Hz, 1H), |
| | tetrahydrofuran-2- | | | | 7.25-7.12 (m, 2H), 5.12 (d, J = |
| | carboxamido)-N,N- | | | | 10.3 Hz, 1H), 4.28 (dd, J = |
| | dimethylimidazo[1,2- | | | | 10.4, 7.5 Hz, 1H), 3.96 (d, J = |
| | a]pyridine-2- | | | | 2.1 Hz, 3H), 3.42 (s, 3H), 2.99 |
| | carboxamide | | | | (s, 3H), 2.86-2.72 (m, 1H), |
| | | | | | 1.62 (s, 3H), 0.75 (d, J = 7.2 |
| | | | | | Hz, 3H) ppm. |

The following compound was made using the method described in Example 7, except that ethyl 6-aminoimidazo[1,2-a]pyridine-2-carboxylate was used as the amine coupling partner in step 8, with K₂CO₃ in DCM instead of triethylamine in 2-MeTHF. Step 8 was followed by ester hydrolysis using General Method 0 and the resulting acid was coupled with tert-butyl piperazine-1-carboxylate, using conditions similar to that described in Example 7, step 8 followed by Boc deprotection using General Method I as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 262 | (2R,3S,4S,5R)-3- | 581.534 | 582 | 3.09 | ¹H NMR (400 MHz, |
| | (3,4-difluoro-2- | | | | Acetonitrile-d₃) δ 9.06 (dd, J = |
| | methoxyphenyl)-4,5- | | | | 2.1, 1.0 Hz, 1H), 8.83 (s, 1H), |
| | dimethyl-N-(2- | | | | 8.02 (d, J = 0.7 Hz, 1H), 7.44 |
| | (piperazine-1- | | | | (dt, J = 9.7, 0.9 Hz, 1H), 7.25 |
| | carbonyl)imidazo[1,2 | | | | (dd, J = 9.7, 2.0 Hz, 1H), 7.17 |
| | -a]pyridin-6-yl)-5- | | | | (ddd, J = 8.4, 5.7, 2.2 Hz, 1H), |
| | (trifluoromethyl) | | | | 6.98 (ddd, J = 10.1, 8.9, 7.6 Hz, |
| | tetrahydrofuran-2- | | | | 1H), 5.07 (d, J = 10.9 Hz, 1H), |
| | carboxamide | | | | 4.22 (dd, J = 10.9, 7.8 Hz, 1H), |
| | | | | | 4.14-3.99 (m, 2H), 3.95 (d, J = |
| | | | | | 2.2 Hz, 3H), 3.68-3.50 (m, |
| | | | | | 2H), 2.87-2.66 (m, 5H), 1.69 |
| | | | | | (d, J = 1.2 Hz, 3H), 0.76 (dq, J = |
| | | | | | 7.4, 2.4 Hz, 3H) ppm. NH not |
| | | | | | observed. |

The following compounds were made using the method described in Example 7, except that different coupling partners were used in the amide coupling step 8 and General Method P was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 20 | (2R,3S,4S,5R)-3- | 463.398 | 464.3 | 3.04 | ¹H NMR (500 MHz, DMSO-d₆) |
| | (3,4-difluoro-2- | | | | δ 10.15 (s, 1H), 7.19 (dd, J = |
| | methoxyphenyl)-N- | | | | 8.5, 5.2 Hz, 2H), 6.11 (s, 1H), |
| | (3-(hydroxymethyl)- | | | | 5.13 (d, J = 10.4 Hz, 1H), 4.92 |
| | 1-methyl-1H- | | | | (t, J = 5.8 Hz, 1H), 4.29 (d, J = |
| | pyrazol-5-yl)-4,5- | | | | 5.7 Hz, 2H), 4.20 (dd, J = 10.4, |
| | dimethyl-5- | | | | 7.6 Hz, 1H), 3.95 (d, J = 2.0 Hz, |
| | (trifluoromethyl) | | | | 3H), 3.50 (s, 3H), 2.75 (t, J = 7.5 |
| | tetrahydrofuran-2- | | | | Hz, 1H), 1.61 (s, 3H), 0.80- |
| | carboxamide | | | | 0.68 (m, 3H) ppm. |
| 263 | (2R,3S,4S,5R)-3- | 463.398 | 464.6 | 3.13 | ¹H NMR (500 MHz, DMSO-d₆) |
| | (3,4-difluoro-2- | | | | δ 10.62 (s, 1H), 7.23-7.12 (m, |
| | methoxyphenyl)-N- | | | | 2H), 6.38 (s, 1H), 5.23 (s, 1H), |
| | (5-(hydroxymethyl)- | | | | 5.06 (d, J = 10.6 Hz, 1H), 4.42 |
| | 1-methyl-1H- | | | | (s, 2H), 4.20 (dd, J = 10.7, 7.6 |
| | pyrazol-3-yl)-4,5- | | | | Hz, 1H), 3.94 (d, J = 1.9 Hz, |
| | dimethyl-5- | | | | 3H), 3.67 (s, 3H), 2.72 (p, J = |
| | (trifluoromethyl) | | | | 7.5 Hz, 1H), 1.58 (s, 3H), 0.70 |
| | tetrahydrofuran-2- | | | | (dd, J = 7.3, 2.6 Hz, 3H) ppm. |
| | carboxamide | | | | |
| 264 | (2R,3S,4S,5R)-3- | 499.43 | | 3.15 | ¹H NMR (500 MHz, DMSO-d₆) |
| | (3,4-difluoro-2- | | | | δ 10.29 (s, 1H), 9.12 (dd, J = |
| | methoxyphenyl)-N- | | | | 2.1, 0.9 Hz, 1H), 7.86-7.75 (m, |
| | (2- | | | | 1H), 7.43 (dt, J = 9.6, 0.8 Hz, |
| | (hydroxymethyl) | | | | 1H), 7.26-7.05 (m, 3H), 5.20- |
| | imidazo[1,2-a]pyridin-6- | | | | 5.04 (m, 2H), 4.54 (dd, J = 5.6, |
| | yl)-4,5-dimethyl-5- | | | | 0.8 Hz, 2H), 4.27 (dd, J = 10.4, |
| | (trifluoromethyl) | | | | 7.6 Hz, 1H), 3.96 (d, J = 2.1 Hz, |
| | tetrahydrofuran-2- | | | | 3H), 2.77 (t, J = 7.5 Hz, 1H), |
| | carboxamide | | | | 1.61 (s, 3H), 0.79-0.66 (m, 3H) |
| | | | | | ppm. |

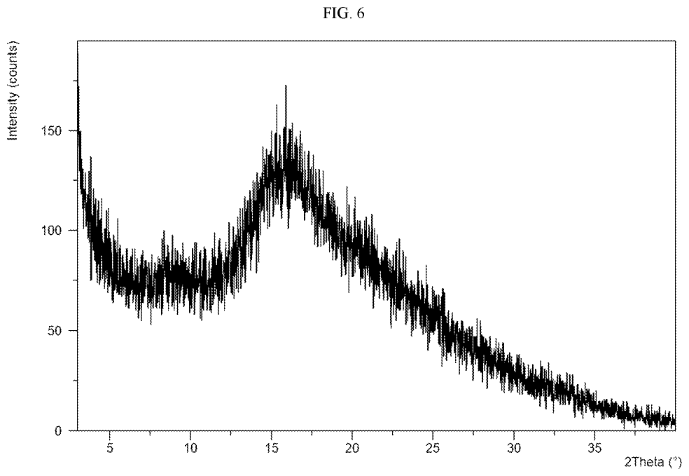

Compound 263 was analyzed by X-ray powder diffraction and determined to be amorphous (see FIG. 6).

The following compound was made using the method described in Example 7, except that 2-[[tert-butyl(dimethyl)silyl]oxymethyl]pyridin-4-amine was used as the amine coupling partner in step 8. Deprotection using General Method N followed by General Method S were used as the final steps. In the

| Cmpd | | LC/MS | Found | MS | |
|---|---|---|---|---|---|
| No. | Compound Name | (m/z calc.) | M + 1 | r.t. | NMR (shifts in ppm) |
| 23 | (2R,3S,4S,5R)-3- | 529.5 | 530.3 | 3.20 | ¹H NMR (500 MHz, DMSO-d₆) |
| | (3,4-difluoro-2- | | | | δ 10.55 (s, 1H), 8.35 (d, J = 5.5 |
| | methoxyphenyl)-4,5- | | | | Hz, 1H), 7.64 (d, J = 2.0 Hz, |
| | dimethyl-N-(2- | | | | 1H), 7.51 (dd, J = 5.6, 2.1 Hz, |
| | (((tetrahydrofuran-3- | | | | 1H), 7.30-7.06 (m, 2H), 5.09 |
| | yl)amino)methyl) | | | | (d, J = 10.3 Hz, 1H), 4.25 (dd, J = |
| | pyridin-4-yl)-5- | | | | 10.3, 7.6 Hz, 1H), 3.96 (d, J = |
| | (trifluoromethyl) | | | | 2.0 Hz, 3H), 3.82-3.58 (m, |
| | tetrahydrofuran-2- | | | | 5H), 3.44 (dd, J = 8.6, 4.2 Hz, |
| | carboxamide | | | | 1H), 3.29 (dd, J = 5.2, 1.7 Hz, |
| | | | | | 1H), 2.78 (p, J = 7.5 Hz, 1H), |
| | | | | | 2.39 (d, J = 22.7 Hz, 1H), 1.93 |
| | | | | | (dq, J = 12.5, 7.2 Hz, 1H), 1.74- |
| | | | | | 1.63 (m, 1H), 1.60 (s, 3H), 0.73 |
| | | | | | (dd, J = 7.3, 2.4 Hz, 3H) ppm. |

The following compound was made using the method described in Example 7, except that 2-[[tert-butyl(dimethyl)silyl]oxymethyl]pyridin-4-amine was used as the amine coupling partner in step 8. Deprotection using General Method N followed by General Method S, using 2-(methylamino)ethanol as the amine in step 2, were used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 265 | (2R,3S,4S,5R)-3- | 517.489 | | 3.34 | ¹H NMR (500 MHz, Methanol- |
| | (3,4-difluoro-2- | | | | d₄) δ 8.40 (dt, J = 12.1, 6.1 Hz, |
| | methoxyphenyl)-N- | | | | 1H), 7.93-7.75 (m, 1H), 7.63 |
| | (2-(((2- | | | | (qd, J = 7.0, 6.2, 3.3 Hz, 1H), |
| | hydroxyethyl)(methyl) | | | | 7.21-7.07 (m, 1H), 7.00 (q, J = |
| | amino)methyl) | | | | 7.9 Hz, 1H), 5.15-5.05 (m, |
| | pyridin-4-yl)-4,5- | | | | 2H), 4.45-4.26 (m, 1H), 4.02 |
| | dimethyl-5- | | | | (tt, J = 6.4, 2.9 Hz, 3H), 3.83 (s, |
| | (trifluoromethyl) | | | | 2H), 3.73 (t, J = 5.8 Hz, 1H), |
| | tetrahydrofuran-2- | | | | 2.91-2.65 (m, 3H), 2.42 (t, J = |
| | carboxamide | | | | 7.4 Hz, 3H), 1.68 (d, J = 7.6 Hz, |
| | | | | | 3H), 1.04-0.68 (m, 3H) ppm. |

### Example 8

rel-(2R*,3S*,4S*,5R*)-3-(3,4-difluoro-2-methoxyphenyl)-4,5-dimethyl-N-((1R,2S)-2-(1-methyl-1H-pyrazol-4-yl)cyclopropyl)-5-(trifluoromethyl)tetrahydrofuran-2-carboxamide and rel-(2R*,3S*,4S*,5R*)-3-(3,4-difluoro-2-methoxyphenyl)-4,5-dimethyl-N-((1S,2R)-2-(1-methyl-1H-pyrazol-4-yl)cyclopropyl)-5-(trifluoromethyl)tetrahydrofuran-2-carboxamide (266, 267)

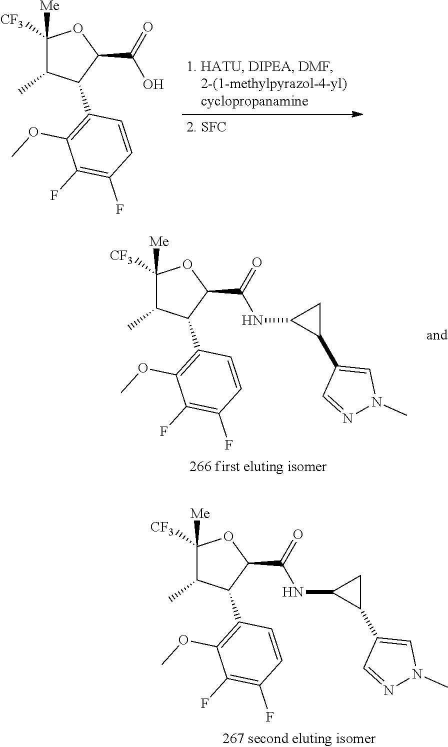

### Step 1:

(2R,3S,4S,5R)-3-(3,4-difluoro-2-methoxy-phenyl)-4,5-dimethyl-5-(trifluoromethyl)tetrahydrofuran-2-carboxylic acid (60 mg, 0.17 mmol), rac-(1R,2S)-2-(1-methyl-1H-pyrazol-4-yl)cyclopropan-1-amine (23 mg, 0.17 mmol), [dimethylamino(triazolo[4,5-b]pyridin-3-yloxy)methylene]-dimethyl-ammonium (Phosphorus Hexafluoride Ion) (97 mg, 0.25 mmol) and DIPEA (65 uL, 0.37 mmol) were suspended in DMF (2 mL) and then stirred overnight at ambient temperature. The resulting mixture was filtered and the liquor purified directly by preparative reverse phase HPLC (basic eluent) to give 2 diastereomers of (2R*,3S*,4S*,5R*)-3-(3,4-difluoro-2-methoxyphenyl)-4,5-dimethyl-N-((1R,2S)-2-(1-methyl-1H-pyrazol-4-yl)cyclopropyl)-5-(trifluoromethyl)tetrahydrofuran-2-carboxamide and (2R*,3S*,4S*,5R*)-3-(3,4-difluoro-2-methoxyphenyl)-4,5-dimethyl-N-((1S,2R)-2-(1-methyl-1H-pyrazol-4-yl)cyclopropyl)-5-(trifluoromethyl)tetrahydrofuran-2-carboxamide (268, 38.2 mg, 47%) as a white solid after lyophilisation. ESI-MS m/z calc. 473.4, found 474.2 (M+1)⁺.

### Step 2:

The two isomers of (2R*,3S*,4S*,5R*)-3-(3,4-difluoro-2-methoxyphenyl)-4,5-dimethyl-N-((1R,2S)-2-(1-methyl-1H-pyrazol-4-yl)cyclopropyl)-5-(trifluoromethyl)tetrahydrofuran-2-carboxamide and (2R*,3S*,4S*,5R*)-3-(3,4-difluoro-2-methoxyphenyl)-4,5-dimethyl-N-((1S,2R)-2-(1-methyl-1H-pyrazol-4-yl)cyclopropyl)-5-(trifluoromethyl)tetrahydrofuran-2-carboxamide (38.2 mg, 0.08 mmol) were separated by chiral SFC using a Chiralpak OD-H column, 5 μm particle size, 25 cm×10 mm from Daicel on a Minigram SFC instrument from Berger Instruments to give:

First Eluting Isomer (rt=2.51 min) rel-(2R*,3S*,4S*,5R*)-3-(3,4-difluoro-2-methoxyphenyl)-4,5-dimethyl-N-((1R,2S)-2-(1-methyl-1H-pyrazol-4-yl)cyclopropyl)-5-(trifluoromethyl)tetrahydrofuran-2-carboxamide (266, 10.4 mg, 27%). ¹H NMR (400 MHz, Chloroform-d) δ 7.18 (d, J=18.9 Hz, 2H), 7.01 (ddd, J=8.3, 5.5, 2.2 Hz, 1H), 6.79 (ddd, J=9.6, 8.9, 7.5 Hz, 1H), 6.65 (d, J=3.1 Hz, 1H), 4.79 (d, J=10.8 Hz, 1H), 3.95-3.85 (m, 4H), 3.74 (s, 3H), 2.68-2.55 (m, 2H), 1.78 (ddd, J=9.6, 6.4, 3.3 Hz, 1H), 1.52 (d, J=1.1 Hz, 3H), 1.06-0.92 (m, 2H), 0.67 (dq, J=7.4, 2.3 Hz, 3H) ppm.

Second Eluting Isomer (rt=3.36 min): rel-(2R*,3S*,4S*,5R*)-3-(3,4-difluoro-2-methoxyphenyl)-4,5-dimethyl-N-((1S,2R)-2-(1-methyl-1H-pyrazol-4-yl)cyclopropyl)-5-(trifluoromethyl)tetrahydrofuran-2-carboxamide (267, 9.7 mg, 25%). ¹H NMR (400 MHz, Chloroform-d) δ 7.21 (s, 1H), 7.12 (s, 1H), 6.99 (ddd, J=8.3, 5.5, 2.2 Hz, 1H), 6.79 (ddd, J=9.7, 8.9, 7.5 Hz, 1H), 6.67-6.62 (m, 1H), 4.78 (d, J=10.8 Hz, 1H), 3.95-3.85 (m, 4H), 3.75 (s, 3H), 2.68-2.55 (m, 2H), 1.77 (ddd, J=9.6, 6.3, 3.3 Hz, 1H), 1.51 (d, J=1.1 Hz, 3H), 1.08-0.92 (m, 2H), 0.67 (dq, J=7.4, 2.3 Hz, 3H) ppm.

The following compounds were made using a method similar to that described in Example 8, except that different amine coupling partners were used in the amide coupling step 1 and step 2 (SFC) was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 269 | (2R,3S,4S,5R)-3-(3,4- | 437.401 | 438.23 | 3.21 | ¹H NMR (400 MHz, |
| | difluoro-2- | | | | Chloroform-d) δ 7.09 (ddd, J = |
| | methoxyphenyl)-4,5- | | | | 8.3, 5.6, 2.2 Hz, 1H), 6.88 |
| | dimethyl-N- | | | | (ddd, J = 9.6, 8.9, 7.5 Hz, |
| | (tetrahydro-2H-pyran- | | | | 1H), 6.50 (d, J = 8.2 Hz, 1H), |
| | 4-yl)-5- | | | | 4.88 (d, J = 10.6 Hz, 1H), |
| | (trifluoromethyl) | | | | 4.03-3.89 (m, 7H), 3.52- |
| | tetrahydrofuran-2- | | | | 3.40 (m, 2H), 2.70 (p, J = 7.6 |
| | carboxamide | | | | Hz, 1H), 1.89 (ddtd, J = 27.0, |
| | | | | | 12.7, 4.5, 2.3 Hz, 2H), 1.64- |
| | | | | | 1.43 (m, 4H), 0.77 (dq, J = |
| | | | | | 7.3, 2.3 Hz, 3H) ppm. |
| 270 | (2R,3S,4S,5R)-N- | 407.375 | 408.23 | 3.48 | |
| | (cyclopropylmethyl)- | | | | |
| | 3-(3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 271 | (2R,3S,4S,5R)-N-(1- | 424.362 | 424.9 | 2.93 | |
| | amino-1-oxopropan-2- | | | | |
| | yl)-3-(3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| | as a mixture of | | | | |
| | epimers at the amino- | | | | |
| | oxopropanyl position | | | | |
| 272 | (2R,3S,4S,5R)-3-(3,4- | 447.399 | 448.18 | 2.95 | |
| | difluoro-2-methoxy- | | | | |
| | phenyl)-N-[2-(1H- | | | | |
| | imidazol-2-yl)ethyl]- | | | | |
| | 4,5-dimethyl-5- | | | | |
| | (trifluoromethyl) | | | | |
| | tetrahydrofuran-2- | | | | |
| | carboxamide | | | | |
| 273 | (2R,3S,4S,5R)-N-(1- | 478.453 | 479.19 | 3.04 | |
| | acetylpiperidin-4-yl)- | | | | |
| | 3-(3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 274 | (2R,3S,4S,5R)-N-(1- | 478.453 | 479.19 | 3.1 | |
| | acetylpiperidin-3-yl)- | | | | |
| | 3-(3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 275 | (2R,3S,4S,5R)-N-(1- | 447.399 | 448.23 | 3.13 | ¹H NMR (400 MHz, |
| | (1H-pyrazol-5- | | | | Chloroform-d) δ 7.56 (d, J = |
| | yl)ethyl)-3-(3,4- | | | | 2.3 Hz, 1H), 7.18-7.05 (m, |
| | difluoro-2- | | | | 2H), 6.95-6.83 (m, 1H), |
| | methoxyphenyl)-4,5- | | | | 6.23 (s, 1H), 5.19 (p, J = 6.8 |
| | dimethyl-5- | | | | Hz, 1H), 4.91 (dd, J = 10.7, |
| | (trifluoromethyl) | | | | 8.8 Hz, 1H), 4.01 (dd, J = |
| | tetrahydrofuran-2- | | | | 9.0, 2.6 Hz, 1H), 4.00-3.89 |
| | carboxamide | | | | (m, 3H), 2.70 (dp, J = 15.4, |
| | as a mixture of | | | | 7.6 Hz, 1H), 1.64-1.53 (m, |
| | epimers at pyrazolyl | | | | 6H), 0.77 (s, 2H), 0.76 (dt, J = |
| | ethyl | | | | 10.0, 2.3 Hz, 2H). ppm |
| 276 | (2R,3S,4S,5R)-3-(3,4- | 465.454 | 466.23 | 3.73 | ¹H NMR (400 MHz, |
| | difluoro-2- | | | | Chloroform-d) δ 7.51 (s, 1H), |
| | methoxyphenyl)-N- | | | | 7.14 (ddd, J = 8.3, 5.6, 2.2 |
| | ((1- | | | | Hz, 1H), 6.89 (ddd, J = 9.7, |
| | (methoxymethyl) | | | | 8.8, 7.5 Hz, 1H), 4.89 (d, J = |
| | cyclobutyl)methyl)- | | | | 10.7 Hz, 1H), 3.99 (d, J = 2.6 |
| | 4,5-dimethyl-5- | | | | Hz, 4H), 3.57-3.38 (m, 3H), |
| | (trifluoromethyl) | | | | 3.41 (s, 3H), 3.29 (dd, J = |
| | tetrahydrofuran-2- | | | | 13.4, 4.6 Hz, 1H), 2.69 (p, J = |
| | carboxamide | | | | 7.6 Hz, 1H), 2.02-1.70 |
| | | | | | (m, 6H), 1.62 (s, 1H), 0.76 |
| | | | | | (dq, J = 7.4, 2.4 Hz, 3H). |
| | | | | | ppm |
| 277 | (2R,3S,4S,5R)-N- | 392.32 | 392.94 | 3.21 | |
| | (cyanomethyl)-3-(3,4- | | | | |
| | difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 278 | (2R,3S,4S,5R)-3-(3,4- | 422.39 | 423.23 | 3.09 | |
| | difluoro-2- | | | | |
| | methoxyphenyl)-4,5- | | | | |
| | dimethyl-N-(1- | | | | |
| | methylazetidin-3-yl)- | | | | |
| | 5- | | | | |
| | (trifluoromethyl) | | | | |
| | tetrahydrofuran-2- | | | | |
| | carboxamide | | | | |
| 279 | (2R,3S,4S,5R)-3-(3,4- | 437.401 | 438.18 | 3.17 | |
| | difluoro-2- | | | | |
| | methoxyphenyl)-4,5- | | | | |
| | dimethyl-N- | | | | |
| | ((tetrahydrofuran-3- | | | | |
| | yl)methyl)-5- | | | | |
| | (trifluoromethyl) | | | | |
| | tetrahydrofuran-2- | | | | |
| | carboxamide | | | | |
| | as a mixture of | | | | |
| | epimers at | | | | |
| | tetrahydrofuranylmethyl | | | | |
| | stereocenter | | | | |
| 280 | (2R,3S,4S,5R)-3-(3,4- | 443.356 | 444.38 | 3.52 | |
| | difluoro-2- | | | | |
| | methoxyphenyl)-N- | | | | |
| | (3,3- | | | | |
| | difluorocyclobutyl)- | | | | |
| | 4,5-dimethyl-5- | | | | |
| | (trifluoromethyl) | | | | |
| | tetrahydrofuran-2- | | | | |
| | carboxamide | | | | |
| 281 | (2R,3S,4S,5R)-3-(3,4- | 450.4 | 451.18 | 2.81 | |
| | difluoro-2- | | | | |
| | methoxyphenyl)-4,5- | | | | |
| | dimethyl-N-((5- | | | | |
| | oxopyrrolidin-3- | | | | |
| | yl)methyl)-5- | | | | |
| | (trifluoromethyl) | | | | |
| | tetrahydrofuran-2- | | | | |
| | carboxamide | | | | |
| | as a mixture of | | | | |
| | epimers at the | | | | |
| | oxopyrrolidinylmethyl | | | | |
| | stereocenter | | | | |
| 282 | (2R,3S,4S,5R)-N-(3- | 452.416 | 453.23 | 2.96 | |
| | amino-2,2-dimethyl-3- | | | | |
| | oxopropyl)-3-(3,4- | | | | |
| | difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 283 | (2R,3S,4S,5R)-3-(3,4- | 461.426 | 462.23 | 3.12 | |
| | difluoro-2- | | | | |
| | methoxyphenyl)-4,5- | | | | |
| | dimethyl-N-(2-(1- | | | | |
| | methyl-1H-pyrazol-4- | | | | |
| | yl)ethyl)-5- | | | | |
| | (trifluoromethyl) | | | | |
| | tetrahydrofuran-2- | | | | |
| | carboxamide | | | | |
| 284 | (2R,3S,4S,5R)-3-(3,4- | 465.454 | 465.91 | 3.4 | ¹H NMR (400 MHz, |
| | difluoro-2- | | | | Chloroform-d) δ 7.10 (ddt, J = |
| | methoxyphenyl)-N- | | | | 8.8, 5.6, 2.7 Hz, 1H), 6.89 |
| | ((5,5- | | | | (td, J = 9.3, 7.5 Hz, 1H), 6.76 |
| | dimethyltetrahydrofuran- | | | | (s, 1H), 4.90 (d, J = 10.8 Hz, |
| | 3-yl)methyl)-4,5- | | | | 1H), 4.04-3.90 (m, 5H), |
| | dimethyl-5- | | | | 3.56 (ddd, J = 8.6, 6.8, 1.6 |
| | (trifluoromethyl) | | | | Hz, 1H), 3.40-3.22 (m, 2H), |
| | tetrahydrofuran-2- | | | | 2.77-2.52 (m, 2H), 1.93 |
| | carboxamide | | | | (ddd, J = 12.6, 8.3, 4.4 Hz, |
| | as a mixture of | | | | 1H), 1.43 (ddd, J = 12.5, 8.1, |
| | epimers at the | | | | 2.6 Hz, 1H), 1.32 (d, J = 4.7 |
| | tetrahydrofuranylmethyl | | | | Hz, 3H), 1.22 (s, 3H), 0.77 |
| | stereocenter | | | | (dq, J = 7.4, 2.4 Hz, 3H) |
| | | | | | ppm. |
| 285 | (2R,3S,4S,5R)-3-(3,4- | 475.452 | 476.19 | 3.13 | ¹H NMR (400 MHz, |
| | difluoro-2- | | | | Chloroform-d) δ 7.10 (ddt, J = |
| | methoxyphenyl)-N-(2- | | | | 8.0, 5.3, 2.8 Hz, 1H), 6.89 |
| | (3,5-dimethyl-1H- | | | | (td, J = 9.3, 7.5 Hz, 1H), 6.71 |
| | pyrazol-4-yl)ethyl)- | | | | (dt, J = 20.2, 6.2 Hz, 1H), |
| | 4,5-dimethyl-5- | | | | 4.89 (dd, J = 11.0, 7.2 Hz, |
| | (trifluoromethyl) | | | | 1H), 4.04-3.89 (m, 5H), |
| | tetrahydrofuran-2- | | | | 3.48 (dq, J = 13.7, 7.0 Hz, |
| | carboxamide | | | | 1H), 3.21 (tt, J = 13.2, 6.6 |
| | | | | | Hz, 1H), 2.77-2.55 (m, 3H), |
| | | | | | 2.45 (s, 1H), 2.36 (s, 4H), |
| | | | | | 2.28 (d, J = 2.6 Hz, 1H), 1.64- |
| | | | | | 1.53 (m, 3H), 0.76 (dd, J = |
| | | | | | 7.4, 2.3 Hz, 3H) ppm. |
| 286 | 5-(((2R,3S,4S,5R)-3- | 476.394 | 477.19 | 2.94 | ¹H NMR (400 MHz, |
| | (3,4-difluoro-2- | | | | Chloroform-d) δ 7.90 (d, J = |
| | methoxyphenyl)-4,5- | | | | 1.0 Hz, 1H), 7.09 (ddd, J = |
| | dimethyl-5- | | | | 8.3, 5.5, 2.2 Hz, 1H), 7.01 (t, |
| | (trifluoromethyl) | | | | J = 6.1 Hz, 1H), 6.89 (ddd, J = |
| | tetrahydrofuran-2- | | | | 9.7, 8.9, 7.5 Hz, 1H), 6.48 |
| | carboxamido)methyl) | | | | (q, J = 0.8 Hz, 1H), 5.78 (s, |
| | furan-3-carboxamide | | | | 2H), 4.93 (d, J = 10.9 Hz, |
| | | | | | 1H), 4.53-4.36 (m, 2H), |
| | | | | | 4.05-3.95 (m, 4H), 2.71 (p, |
| | | | | | J = 7.6 Hz, 1H), 1.66-1.58 |
| | | | | | (m, 3H), 0.78 (dq, J = 7.4, |
| | | | | | 2.3 Hz, 3H) ppm. |
| 287 | (2R,3S,4S,5R)-3-(3,4- | 476.437 | 477.14 | 3.38 | |
| | difluoro-2- | | | | |
| | methoxyphenyl)-N-(2- | | | | |
| | (3,5-dimethylisoxazol- | | | | |
| | 4-yl)ethyl)-4,5- | | | | |
| | dimethyl-5- | | | | |
| | (trifluoromethyl) | | | | |
| | tetrahydrofuran-2- | | | | |
| | carboxamide | | | | |
| 288 | (2R,3S,4S,5R)-3-(3,4- | 477.425 | 478.24 | 3.05 | |
| | difluoro-2- | | | | |
| | methoxyphenyl)-N- | | | | |
| | ((5-(methoxymethyl)- | | | | |
| | 1H-pyrazol-3- | | | | |
| | yl)methyl)-4,5- | | | | |
| | dimethyl-5- | | | | |
| | (trifluoromethyl) | | | | |
| | tetrahydrofuran-2- | | | | |
| | carboxamide | | | | |
| 289 | (2R,3S,4S,5R)-3-(3,4- | 477.425 | 478.19 | 3.55 | |
| | difluoro-2- | | | | |
| | methoxyphenyl)-N- | | | | |
| | ((3-isopropyl-1,2,4- | | | | |
| | oxadiazol-5-yl) | | | | |
| | methyl)-4,5- | | | | |
| | dimethyl-5- | | | | |
| | (trifluoromethyl) | | | | |
| | tetrahydrofuran-2- | | | | |
| | carboxamide | | | | |
| 290 | (2R,3S,4S,5R)-3-(3,4- | 480.426 | 481.24 | 2.95 | ¹H NMR (400 MHz, |
| | difluoro-2- | | | | Chloroform-d) δ 7.08 (ddd, J = |
| | methoxyphenyl)-4,5- | | | | 8.3, 5.6, 2.2 Hz, 1H), 6.99- |
| | dimethyl-N-((4- | | | | 6.84 (m, 2H), 4.92 (d, J = |
| | methyl-5- | | | | 10.8 Hz, 1H), 4.31 (dd, J = |
| | oxomorpholin-2-yl) | | | | 16.4, 6.1 Hz, 1H), 4.22-4.09 |
| | methyl)-5- | | | | (m, 1H), 4.01 (dd, J = 2.7, |
| | (trifluoromethyl) | | | | 1.3 Hz, 5H), 3.95-3.82 (m, |
| | tetrahydrofuran-2- | | | | 1H), 3.60 (dddd, J = 34.2, |
| | carboxamide | | | | 14.2, 6.7, 3.6 Hz, 1H), 3.44- |
| | as a mixture of | | | | 3.20 (m, 2H), 3.14 (dddd, J = |
| | epimers at the | | | | 11.4, 8.1, 3.3, 0.8 Hz, 1H), |
| | stereocenter of the | | | | 2.98 (s, 1H), 2.97 (s, 3H), |
| | oxomorpholine of the | | | | 2.71 (p, J = 7.7 Hz, 1H), 0.78 |
| | N- | | | | (dq, J = 7.4, 2.4 Hz, 3H). |
| | methyloxomorpholine | | | | ppm |
| 291 | (2R,3S,4S,5R)-N-(1- | 483.471 | 484.19 | 3.81 | |
| | benzylcyclopropyl)-3- | | | | |
| | (3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 292 | (2R,3S,4S,5R)-N-(1-(2- | 486.435 | 487.19 | 3.3 | |
| | cyanoethyl)-3-methyl- | | | | |
| | 1H-pyrazol-5-yl)-3- | | | | |
| | (3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| 293 | (2R,3S,4S,5R)-3-(3,4- | 493.51 | 494.24 | 3.21 | |
| | difluoro-2- | | | | |
| | methoxyphenyl)-4,5- | | | | |
| | dimethyl-N-(2-(4- | | | | |
| | methylpiperazin-1-yl) | | | | |
| | propyl)-5- | | | | |
| | (trifluoromethyl) | | | | |
| | tetrahydrofuran-2- | | | | |
| | carboxamide | | | | |
| | as a mixture of | | | | |
| | epimers at the | | | | |
| | stereocenter of the | | | | |
| | piperazine of the N- | | | | |
| | methylpiperazine | | | | |
| 294 | (2R,3S,4S,5R)-3-(3,4- | 494.454 | 495.14 | 3.29 | |
| | difluoro-2- | | | | |
| | methoxyphenyl)-4,5- | | | | |
| | dimethyl-N-(quinolin- | | | | |
| | 6-ylmethyl)-5- | | | | |
| | (trifluoromethyl) | | | | |
| | tetrahydrofuran-2- | | | | |
| | carboxamide | | | | |
| 295 | (2R,3S,4S,5R)-3-(3,4- | 458.422 | 459.23 | 3.18 | |
| | difluoro-2- | | | | |
| | methoxyphenyl)-4,5- | | | | |
| | dimethyl-N-((4- | | | | |
| | methylpyridin-3-yl) | | | | |
| | methyl)-5- | | | | |
| | (trifluoromethyl) | | | | |
| | tetrahydrofuran-2- | | | | |
| | carboxamide | | | | |
| 296 | (2R,3S,4S,5R)-3-(3,4- | 461.426 | 462.23 | 3.18 | |
| | difluoro-2- | | | | |
| | methoxyphenyl)-4,5- | | | | |
| | dimethyl-N-(1-(1- | | | | |
| | methyl-1H-pyrazol-4- | | | | |
| | yl)ethyl)-5- | | | | |
| | (trifluoromethyl) | | | | |
| | tetrahydrofuran-2- | | | | |
| | carboxamide | | | | |
| | as a mixture of | | | | |
| | epimers at the | | | | |
| | pyrazolylethyl | | | | |
| | stereocenter | | | | |
| 297 | (2R,3S,4S,5R)-N-(1- | 464.426 | 465.28 | 3.16 | |
| | (cyclopropylamino)-1- | | | | |
| | oxopropan-2-yl)-3- | | | | |
| | (3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| | mixture of epimers | | | | |
| 298 | (2R,3S,4S,5R)-N-(1- | 464.426 | 465.28 | 2.94 | |
| | acetylpyrrolidin-3-yl)- | | | | |
| | 3-(3,4-difluoro-2- | | | | |
| | methoxyphenyl)-4,5- | | | | |
| | dimethyl-5- | | | | |
| | (trifluoromethyl) | | | | |
| | tetrahydrofuran-2- | | | | |
| | carboxamide | | | | |
| | mixture of epimers | | | | |
| 299 | (2R,3S,4S,5R)-3-(3,4- | 464.426 | 465.34 | 3.18 | |
| | difluoro-2- | | | | |
| | methoxyphenyl)-4,5- | | | | |
| | dimethyl-N-(2- | | | | |
| | oxoazepan-3-yl)-5- | | | | |
| | (trifluoromethyl) | | | | |
| | tetrahydrofuran-2- | | | | |
| | carboxamide | | | | |
| | mixture of epimers | | | | |
| 300 | (2R,3S,4S,5R)-3-(3,4- | 465.454 | 466.39 | 3.35 | |
| | difluoro-2- | | | | |
| | methoxyphenyl)-4,5- | | | | |
| | dimethyl-N-((4- | | | | |
| | methyltetrahydro-2H- | | | | |
| | pyran-4-yl)methyl)-5- | | | | |
| | (trifluoromethyl) | | | | |
| | tetrahydrofuran-2- | | | | |
| | carboxamide | | | | |
| 301 | (2R,3S,4S,5R)-3-(3,4- | 477.425 | 478.24 | 2.89 | |
| | difluoro-2- | | | | |
| | methoxyphenyl)-N-(2- | | | | |
| | hydroxy-2-(1-methyl- | | | | |
| | 1H-pyrazol-4-yl) | | | | |
| | ethyl)-4,5-dimethyl- | | | | |
| | 5- | | | | |
| | (trifluoromethyl) | | | | |
| | tetrahydrofuran-2- | | | | |
| | carboxamide | | | | |
| | mixture of epimers | | | | |
| 302 | (2R,3S,4S,5R)-3-(3,4- | 479.441 | 480.34 | 2.79 | |
| | difluoro-2- | | | | |
| | methoxyphenyl)-4,5- | | | | |
| | dimethyl-N-(2-(3- | | | | |
| | oxopiperazin-1-yl) | | | | |
| | ethyl)-5- | | | | |
| | (trifluoromethyl) | | | | |
| | tetrahydrofuran-2- | | | | |
| | carboxamide | | | | |
| 303 | 4-((2R,3S,4S,5R)-3- | 479.441 | 480.29 | 2.89 | ¹H NMR (400 MHz, |
| | (3,4-difluoro-2- | | | | Chloroform-d) δ 7.09 (ddd, J = |
| | methoxyphenyl)-4,5- | | | | 8.3, 5.5, 2.2 Hz, 1H), 6.89 |
| | dimethyl-5- | | | | (td, J = 9.3, 7.5 Hz, 1H), 6.50 |
| | (trifluoromethyl) | | | | (d, J = 8.0 Hz, 1H), 4.88 (d, J = |
| | tetrahydrofuran-2- | | | | 10.7 Hz, 1H), 4.55 (s, 2H), |
| | carboxamido) | | | | 4.08-3.85 (m, 5H), 2.96 |
| | piperidine-1- | | | | (tdd, J = 13.6, 11.8, 2.9 Hz, |
| | carboxamide | | | | 2H), 2.70 (p, J = 7.7 Hz, 1H), |
| | | | | | 2.00 (d, J = 13.4 Hz, 1H), |
| | | | | | 1.92 (s, 1H), 1.61 (d, J = 1.1 |
| | | | | | Hz, 3H), 1.46 (qt, J = 11.9, |
| | | | | | 4.6 Hz, 2H), 0.78 (dq, J = |
| | | | | | 7.4, 2.3 Hz, 3H) ppm. |
| 304 | (2R,3S,4S,5R)-3-(3,4- | 480.426 | 480.87 | 3.05 | |
| | difluoro-2- | | | | |
| | methoxyphenyl)-4,5- | | | | |
| | dimethyl-N-(2- | | | | |
| | morpholino-2- | | | | |
| | oxoethyl)-5- | | | | |
| | (trifluoromethyl) | | | | |
| | tetrahydrofuran-2- | | | | |
| | carboxamide | | | | |
| 305 | (2R,3S,4S,5R)-3-(3,4- | 483.471 | 484.29 | 3.86 | |
| | difluoro-2- | | | | |
| | methoxyphenyl)-4,5- | | | | |
| | dimethyl-N-((1- | | | | |
| | phenylcyclopropyl) | | | | |
| | methyl)-5- | | | | |
| | (trifluoromethyl) | | | | |
| | tetrahydrofuran-2- | | | | |
| | carboxamide | | | | |
| 306 | (2R,3S,4S,5R)-3-(3,4- | 485.444 | 486.3 | 3.5 | ¹H NMR (400 MHz, |
| | difluoro-2- | | | | Chloroform-d) δ 7.52-7.45 |
| | methoxyphenyl)-4,5- | | | | (m, 2H), 7.40 (ddd, J = 7.8, |
| | dimethyl-N-(3- | | | | 6.9, 1.3 Hz, 2H), 7.36-7.27 |
| | phenyloxetan-3-yl)-5- | | | | (m, 2H), 7.03 (ddd, J = 8.3, |
| | (trifluoromethyl) | | | | 5.6, 2.2 Hz, 1H), 6.92-6.80 |
| | tetrahydrofuran-2- | | | | (m, 1H), 5.03 (dd, J = 6.8, |
| | carboxamide | | | | 3.9 Hz, 2H), 5.02-4.89 (m, |
| | | | | | 3H), 4.07 (dd, J = 10.9, 8.1 |
| | | | | | Hz, 1H), 3.95 (d, J = 2.5 Hz, |
| | | | | | 3H), 2.74 (p, J = 7.7 Hz, 1H), |
| | | | | | 1.69 (s, 2H), 0.80 (dq, J = |
| | | | | | 7.4, 2.4 Hz, 3H) ppm. |
| 307 | (2R,3S,4S,5R)-3-(3,4- | 486.453 | 487.14 | 3.16 | ¹H NMR (400 MHz, |
| | difluoro-2- | | | | Chloroform-d) δ 7.05 (ddd, J = |
| | methoxyphenyl)-4,5- | | | | 8.3, 5.6, 2.2 Hz, 1H), 6.97 |
| | dimethyl-N-(1- | | | | (d, J = 7.8 Hz, 1H), 6.88 |
| | (methylsulfonyl) | | | | (ddd, J = 9.6, 8.9, 7.4 Hz, |
| | azetidin-3-yl)-5- | | | | 1H), 4.89 (d, J = 11.0 Hz, |
| | (trifluoromethyl) | | | | 1H), 4.66 (qt, J = 7.7, 6.3 Hz, |
| | tetrahydrofuran-2- | | | | 1H), 4.19-4.08 (m, 2H), |
| | carboxamide | | | | 4.05-3.89 (m, 6H), 2.92 (s, |
| | | | | | 3H), 2.72 (p, J = 7.8 Hz, 1H), |
| | | | | | 1.64 (d, J = 7.6 Hz, 3H), 0.77 |
| | | | | | (dq, J = 7.1, 2.2 Hz, 3H) |
| | | | | | ppm. |
| 308 | (2R,3S,4S,5R)-3-(3,4- | 489.451 | 490.35 | 3.87 | |
| | difluoro-2- | | | | |
| | methoxyphenyl)-N-(1- | | | | |
| | (3-fluoro-4- | | | | |
| | methylphenyl)ethyl)- | | | | |
| | 4,5-dimethyl-5- | | | | |
| | (trifluoromethyl) | | | | |
| | tetrahydrofuran-2- | | | | |
| | carboxamide | | | | |
| | mixture of epimers | | | | |
| 309 | (2R,3S,4S,5R)-3-(3,4- | 457.412 | 458.03 | 3.15 | ¹H NMR (400 MHz, |
| | difluoro-2- | | | | Chloroform-d) δ 7.20 (d, J = |
| | methoxyphenyl)-N- | | | | 6.8 Hz, 1H), 7.04 (ddd, J = |
| | (1,1-dioxidothietan-3- | | | | 8.3, 5.6, 2.2 Hz, 1H), 6.95- |
| | yl)-4,5-dimethyl-5- | | | | 6.83 (m, 1H), 4.91 (d, J = |
| | (trifluoromethyl) | | | | 11.0 Hz, 1H), 4.66 (tdt, J = |
| | tetrahydrofuran-2- | | | | 8.5, 6.7, 3.8 Hz, 1H), 4.60- |
| | carboxamide | | | | 4.44 (m, 2H), 4.10-3.92 (m, |
| | | | | | 6H), 2.73 (p, J = 7.6 Hz, 1H), |
| | | | | | 1.63 (d, J = 17.5 Hz, 3H), |
| | | | | | 0.78 (dq, J = 7.5, 2.4 Hz, 3H) |
| | | | | | ppm. |

The following compound was made using a method similar to that described in Example 8, except that tert-butyl N-[3-(4-aminopyrimidin-2-yl)oxetan-3-yl]carbamate was used as the amine in step 1 with heating at 60° C. SFC purification step 2 was omitted and General Method I, using neat TFA, was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 310 | (2R,3S,4S,5R)-N-(2- | 502.434 | 503.3 | 3.14 | ¹H NMR (500 MHz, DMSO- |
| | (3-aminooxetan-3- | | | | d₆) δ 11.21-10.99 (m, 1H), |
| | yl)pyrimidin-4-yl)-3- | | | | 8.72 (d, J = 5.7 Hz, 1H), 7.93 |
| | (3,4-difluoro-2- | | | | (d, J = 5.7 Hz, 1H), 7.25-7.09 |
| | methoxyphenyl)-4,5- | | | | (m, 2H), 5.29 (d, J = 10.3 Hz, |
| | dimethyl-5- | | | | 1H), 4.94 (t, J = 6.0 Hz, 2H), |
| | (trifluoromethyl)- | | | | 4.55 (d, J = 5.7 Hz, 2H), 4.27 |
| | tetrahydrofuran-2- | | | | (dd, J = 10.3, 7.7 Hz, 1H), 3.95 |
| | carboxamide | | | | (d, J = 2.1 Hz, 3H), 2.78 (p, J = |
| | | | | | 7.5 Hz, 1H), 2.69 (s, 2H), 1.59 |
| | | | | | (s, 3H), 0.71 (d, J = 7.4 Hz, |
| | | | | | 3H). ppm |

### Example 9

(2R,3S,4S,5R)-3-(3,4-difluoro-2-methoxyphenyl)-N-(1-(difluoromethyl)-3-methyl-1H-pyrazol-4-yl)-4,5-dimethyl-5-(trifluoromethyl)tetrahydrofuran-2-carboxamide (311)

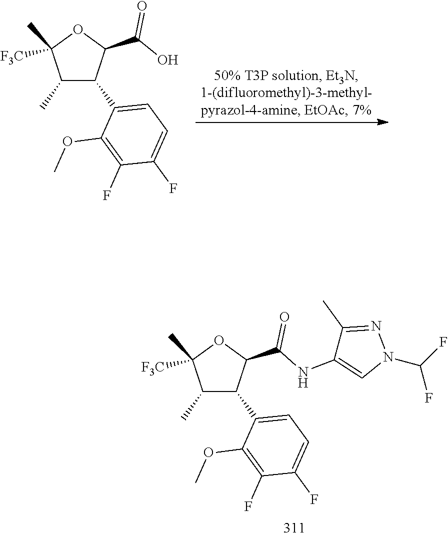

To a solution of (2R,3S,4S,5R)-3-(3,4-difluoro-2-methoxyphenyl)-4,5-dimethyl-5-(trifluoromethyl)tetrahydrofuran-2-carboxylic acid in ethyl acetate (10 mL) and triethylamine (87 μL, 0.6242 mmol) was added 50% T3P solution (375 μL, 1.260 mmol), triethylamine (87 μL, 0.6242 mmol) and 1-(difluoromethyl)-3-methyl-pyrazol-4-amine (Hydrochloride salt) (69 mg, 0.3758 mmol). The reaction mixture was stirred at ambient temperature for 4 hours. The reaction mixture was partitioned between TBME (20 ml) and water (40 mL). The aqueous layer was further extracted with TBME (10 mL). Combined organic fractions were washed with brine (1×10 mL), dried over magnesium sulfate and concentrated to dryness. The product was purified by flash column chromatography (40 g SiO₂, 0 to 100% EtOAc in heptane) to give (2R,3S,4S,5R)-3-(3,4-difluoro-2-methoxyphenyl)-N-(1-(difluoromethyl)-3-methyl-1H-pyrazol-4-yl)-4,5-dimethyl-5-(trifluoromethyl)tetrahydrofuran-2-carboxamide (311, 11 mg, 7%) as a white solid. ¹H NMR (500 MHz, DMSO-d₆) δ 9.97 (s, 1H), 8.32 (s, 1H), 7.63 (s, 1H), 7.21-7.12 (m, 2H), 5.17 (d, J=10.5 Hz, 1H), 4.23 (dd, J=10.5, 7.5 Hz, 1H), 3.95 (s, 3H), 2.76 (p, J=7.4 Hz, 1H), 2.18 (s, 3H), 1.59 (s, 3H), 0.74 (d, J=7.4 Hz, 3H) ppm. ESI-MS m/z calc. 483.13928, found 484.6 (M+1)⁺; Retention time: 3.6 minutes.

The following compounds were made using a method similar to that described in Example 9, except that different amines were used in step 1. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 312 | (2R,3S,4S,5R)-3- | 528.216 | 529.0 | | ¹H NMR (400 MHz, DMSO- |
| | (3,4-difluoro-2- | | | | d₆) δ 10.28 (s, 1H), 7.99 (d, J = |
| | methoxyphenyl)-4,5- | | | | 5.6 Hz, 1H), 7.15 (dt, J = |
| | dimethyl-N-(2-(4- | | | | 11.6, 8.8 Hz, 3H), 6.84 (dd, J = |
| | methylpiperazin-1- | | | | 5.6, 1.6 Hz, 1H), 5.07 (d, J = |
| | yl)pyridin-4-yl)-5- | | | | 10.3 Hz, 1H), 4.23 (dd, J = |
| | (trifluoromethyl)- | | | | 10.3, 7.6 Hz, 1H), 3.95 (d, J = |
| | tetrahydrofuran-2- | | | | 2.2 Hz, 3H), 3.40 (t, J = 4.9 |
| | carboxamide | | | | Hz, 4H), 3.29 (s, 1H), 2.76 |
| | | | | | (p, J = 7.5 Hz, 1H), 2.38 (s, |
| | | | | | 3H), 2.21 (s, 3H), 1.59 (s, |
| | | | | | 3H), 0.72 (d, J = 7.0 Hz, 3H) |
| | | | | | ppm. |
| 313 | (2R,3S,4S,5R)-3- | 433.372 | 434 | 3.04 | ¹H NMR (400 , DMSO- |
| | (3,4-difluoro-2- | | | | d₆) δ 9.89 (s, 1H), 7.47 (dd, J = |
| | methoxyphenyl)-4,5- | | | | 1.2, 0.5 Hz, 1H), 7.25- |
| | dimethyl-N-(1- | | | | 7.12 (m, 2H), 6.76 (d, J = 1.1 |
| | methyl-1H-imidazol- | | | | Hz, 1H), 5.12 (d, J = 10.5 |
| | 5-yl)-5- | | | | Hz, 1H), 4.18 (dd, J = 10.5, |
| | (trifluoromethyl)- | | | | 7.6 Hz, 1H), 3.94 (d, J = 2.2 |
| | tetrahydrofuran-2- | | | | Hz, 3H), 2.74 (p, J = 7.5 Hz, |
| | carboxamide | | | | 1H), 1.61 (s, 3H), 0.77-0.70 |
| | | | | | (m, 3H) ppm. |
| 314 | (2R,3S,4S,5R)-3- | 483.431 | 484 | 3.42 | ¹H NMR (400 MHz, DMSO- |
| | (3,4-difluoro-2- | | | | d₆) δ 10.29 (s, 1H), 8.68 (q, J = |
| | methoxy-phenyl)- | | | | 1.2 Hz, 1H), 7.51 (dd, J = |
| | 4,5-dimethyl-N-(3- | | | | 9.6, 1.0 Hz, 1H), 7.24 (d, J = |
| | methylimidazo[1,5- | | | | 1.0 Hz, 1H), 7.23-7.10 (m, |
| | alpyridin-6-yl)-5- | | | | 2H), 6.73 (dd, J = 9.6, 1.6 |
| | (trifluoromethyl)- | | | | Hz, 1H), 5.10 (d, J = 10.4 |
| | tetrahydrofuran-2- | | | | Hz, 1H), 4.27 (dd, J = 10.4, |
| | carboxamide | | | | 7.5 Hz, 1H), 3.97 (d, J = 2.3 |
| | | | | | Hz, 3H), 2.78 (p, J = 7.4 Hz, |
| | | | | | 1H), 1.61 (s, 3H), 0.85-0.63 |
| | | | | | (m, 3H). |
| 315 | (2R,3S,4S,5R)-3- | 483.431 | 484 | 3.31 | ¹H NMR (400 MHz, DMSO- |
| | (3,4-difluoro-2- | | | | d₆) δ 10.24 (s, 1H), 8.04 (dt, J = |
| | methoxyphenyl)-4,5- | | | | 7.6, 1.0 Hz, 1H), 7.91 (dd, |
| | dimethyl-N-(3- | | | | J = 2.1, 1.0 Hz, 1H), 7.24- |
| | methylimidazo[1,5- | | | | 7.12 (m, 2H), 7.11 (d, J = 0.9 |
| | a]pyridin-7-yl)-5- | | | | Hz, 1H), 6.74 (dd, J = 7.6, |
| | (trifluoromethyl)- | | | | 2.0 Hz, 1H), 5.05 (d, J = 10.3 |
| | tetrahydrofuran-2- | | | | Hz, 1H), 4.26 (dd, J = 10.3, |
| | carboxamide | | | | 7.6 Hz, 1H), 3.96 (d, J = 2.2 |
| | | | | | Hz, 3H), 2.77 (p, J = 7.5 Hz, |
| | | | | | 1H), 2.54 (d, J = 4.4 Hz, 3H), |
| | | | | | 1.61 (s, 3H), 0.77-0.70 (m, |
| | | | | | 3H) ppm. |
| 316 | (2R,3S,4S,5R)-3- | 510.456 | 512 | 3.34 | ¹H NMR (400 MHz, DMSO- |
| | (3,4-difluoro-2- | | | | d₆) δ 10.76 (s, 1H), 8.43 (d, J = |
| | methoxyphenyl)-4,5- | | | | 5.6 Hz, 1H), 7.81 (d, J = |
| | dimethyl-N-(2-(2- | | | | 1.8 Hz, 1H), 7.63 (dd, J = |
| | methyl-1H-imidazol- | | | | 5.6, 1.8 Hz, 1H), 7.45 (d, J = |
| | 1-yl)pyridin-4-yl)-5- | | | | 1.5 Hz, 1H), 7.16 (dd, J = |
| | (trifluoromethyl)- | | | | 10.0, 6.5 Hz, 2H), 6.90 (d, J = |
| | tetrahydrofuran-2- | | | | 1.5 Hz, 1H), 5.13 (d, J = |
| | carboxamide | | | | 10.1 Hz, 1H), 4.27 (dd, J = |
| | | | | | 10.1, 7.7 Hz, 1H), 3.95 (d, J = |
| | | | | | 2.0 Hz, 3H), 2.78 (t, J = 7.6 |
| | | | | | Hz, 1H), 2.45 (s, 3H), 1.60 |
| | | | | | (s, 3H), 0.73 (dd, J = 7.6, 2.3 |
| | | | | | Hz, 3H) ppm. |
| 317 | (2R,3S,4S,5R)-3- | 473.436 | 474 | 3.19 | ¹H NMR (400 MHz, DMSO- |
| | (3,4-difluoro-2- | | | | d₆) δ 9.78 (s, 1H), 7.25-7.12 |
| | methoxyphenyl)-4,5- | | | | (m, 2H), 6.66 (s, 1H), 5.10 |
| | dimethyl-N-(5,6,7,8- | | | | (d, J = 10.6 Hz, 1H), 4.17 |
| | tetrahydroimidazo[1, | | | | (dd, J = 10.6, 7.6 Hz, 1H), |
| | 2-a]pyridin-3-yl)-5- | | | | 3.94 (d, J = 2.2 Hz, 3H), 3.50 |
| | (trifluoromethyl)- | | | | (t, J = 5.9 Hz, 2H), 2.74 (p, J = |
| | tetrahydrofuran-2- | | | | 7.6 Hz, 1H), 2.65 (t, J = 6.2 |
| | carboxamide | | | | Hz, 2H), 1.85-1.73 (m, 4H), |
| | | | | | 1.60 (s, 3H), 0.77-0.69 (m, |
| | | | | | 3H) ppm. |
| 318 | (2R,3S,4S,5R)-3- | 433.372 | 434 | 3.11 | ¹H NMR (400 MHz, DMSO- |
| | (3,4-difluoro-2- | | | | d₆) δ 10.60 (s, 1H), 7.38 (d, J = |
| | methoxyphenyl)-4,5- | | | | 1.5 Hz, 1H), 7.25-7.11 |
| | dimethyl-N-(1- | | | | (m, 3H), 5.09 (d, J = 10.8 Hz, |
| | methyl-1H-imidazol- | | | | 1H), 4.22 (dd, J = 10.7, 7.5 |
| | 4-yl)-5- | | | | Hz, 1H), 3.94 (d, J = 2.2 Hz, |
| | (trifluoromethyl)- | | | | 3H), 3.58 (s, 3H), 2.73 (q, J = |
| | tetrahydrofuran-2- | | | | 7.4 Hz, 1H), 1.58 (s, 3H), |
| | carboxamide | | | | 0.70 (d, J = 6.8 Hz, 3H) ppm. |
| 319 | (2R,3S,4S,5R)-3- | 447.399 | 448 | 3.08 | ¹H NMR (400 MHz, DMSO- |
| | (3,4-difluoro-2- | | | | d₆) δ 9.61 (s, 1H), 7.39 (s, |
| | methoxyphenyl)-N- | | | | 1H), 7.29-7.13 (m, 2H), |
| | (1,4-dimethyl-1H- | | | | 5.10 (d, J = 10.6 Hz, 1H), |
| | imidazol-5-yl)-4,5- | | | | 4.16 (dd, J = 10.6, 7.5 Hz, |
| | dimethyl-5- | | | | 1H), 3.93 (d, J = 2.1 Hz, 3H), |
| | (trifluoromethyl)- | | | | 3.23 (s, 3H), 2.73 (q, J = 7.5 |
| | tetrahydrofuran-2- | | | | Hz, 1H), 1.82 (s, 3H), 1.62 |
| | carboxamide | | | | (s, 3H), 0.78-0.71 (m, 3H) |
| | | | | | ppm. |
| 320 | (2R,3S,4S,5R)-3- | 473.436 | 474 | 3.54 | ¹H NMR (400 MHz, DMSO- |
| | (3,4-difluoro-2- | | | | d₆) δ 10.29 (s, 1H), 7.95 (d, J = |
| | methoxyphenyl)-N- | | | | 5.6 Hz, 1H), 7.24-7.07 |
| | (2-(dimethylamino)- | | | | (m, 2H), 6.95 (d, J = 1.7 Hz, |
| | pyridin-4-yl)-4,5- | | | | 1H), 6.78 (dd, J = 5.6, 1.7 |
| | dimethyl-5- | | | | Hz, 1H), 5.06 (d, J = 10.3 |
| | (trifluoromethyl)- | | | | Hz, 1H), 4.24 (dd, J = 10.3, |
| | tetrahydrofuran-2- | | | | 7.6 Hz, 1H), 3.96 (d, J = 2.2 |
| | carboxamide | | | | Hz, 3H), 2.97 (s, 6H), 2.77 |
| | | | | | (p, J = 7.5, 7.1 Hz, 1H), 1.59 |
| | | | | | (s, 3H), 0.76-0.69 (m, 3H) |
| | | | | | ppm. |
| 321 | (2R,3S,4S,5R)-3- | 475.409 | 476.3 | 3.27 | ¹H NMR (500 MHz, DMSO- |
| | (3,4-difluoro-2- | | | | d₆) δ 10.71 (s, 1H), 7.25- |
| | methoxyphenyl)-N- | | | | 7.11 (m, 2H), 6.28 (s, 1H), |
| | (6,7-dihydro-4H- | | | | 5.07 (d, J = 10.7 Hz, 1H), |
| | pyrazolo[5,1- | | | | 4.77-4.65 (m, 2H), 4.21 (dd, |
| | c][1,4]oxazin-2-yl)- | | | | J = 10.7, 7.6 Hz, 1H), 4.07- |
| | 4,5-dimethyl-5- | | | | 4.00 (m, 2H), 4.00-3.95 (m, |
| | (trifluoromethyl)- | | | | 2H), 3.94 (d, J = 2.0 Hz, 3H), |
| | tetrahydrofuran-2- | | | | 2.72 (q, J = 7.5 Hz, 1H), 1.58 |
| | carboxamide | | | | (s, 3H), 0.76-0.63 (m, 3H) |
| | | | | | ppm. |
| 322 | (2R,3S,4S,5R)-3- | 447.399 | 448 | 3.04 | ¹H NMR (400 MHz, DMSO- |
| | (3,4-difluoro-2- | | | | d₆) δ 9.65 (s, 1H), 7.36 (s, |
| | methoxyphenyl)-N- | | | | 1H), 7.25-7.12 (m, 2H), |
| | (1,5-dimethyl-1H- | | | | 5.01 (d, J = 10.6 Hz, 1H), |
| | imidazol-4-yl)-4,5- | | | | 4.17 (dd, J = 10.6, 7.7 Hz, |
| | dimethyl-5- | | | | 1H), 3.94 (d, J = 2.1 Hz, 3H), |
| | (trifluoromethyl)- | | | | 3.47 (s, 3H), 2.72 (q, J = 7.5 |
| | tetrahydrofuran-2- | | | | Hz, 1H), 1.83 (s, 3H), 1.59 |
| | carboxamide | | | | (s, 3H), 0.75-0.68 (m, 3H) |
| | | | | | ppm. |
| 323 | ((2R,3S,4S,5R)-3- | 456.406 | 457.3 | 3.29 | ¹H NMR (500 MHz, DMSO- |
| | (3,4-difluoro-2- | | | | d₆) δ 8.62 (d, J = 28.5 Hz, |
| | methoxyphenyl)-4,5- | | | | 1H), 8.49 (d, J = 5.1 Hz, 1H), |
| | dimethyl-5- | | | | 7.44 (dd, J = 34.5, 5.0 Hz, |
| | (trifluoromethyl)- | | | | 1H), 7.16-7.05 (m, 2H), |
| | tetrahydrofuran-2-yl) | | | | 5.30 (dd, J = 9.9, 6.8 Hz, |
| | (1,3-dihydro-2H- | | | | 1H), 5.24 (dd, J = 15.4, 5.2 |
| | pyrrolo[3,4- | | | | Hz, 1H), 4.98 (t, J = 14.9 Hz, |
| | c]pyridin-2- | | | | 1H), 4.70 (d, J = 9.7 Hz, 2H), |
| | yl)methanone | | | | 4.41-4.34 (m, 1H), 3.97 (d, |
| | | | | | J = 1.9 Hz, 3H), 2.73 (p, J = |
| | | | | | 7.5 Hz, 1H), 1.51 (s, 3H), |
| | | | | | 0.80-0.69 (m, 3H) ppm. |
| 324 | (2R,3S,4S,5R)-3- | 476.46 | 477.1 | 3.68 | ¹H NMR (500 MHz, |
| | (3,4-difluoro-2- | | | | Chloroform-d) δ 8.46 (d, J = |
| | methoxyphenyl)-4,5- | | | | 2.6 Hz, 1H), 8.26 (s, 1H), |
| | dimethyl-N-(6- | | | | 7.94 (dd, J = 8.7, 2.7 Hz, |
| | (methylthio)pyridin- | | | | 1H), 7.13 (d, J = 8.8 Hz, 1H), |
| | 3-yl)-5- | | | | 7.09 (ddd, J = 8.1, 5.5, 2.1 |
| | (trifluoromethyl)- | | | | Hz, 1H), 6.90 (td, J = 9.3, 7.5 |
| | tetrahydrofuran-2- | | | | Hz, 1H), 5.01 (d, J = 10.8 |
| | carboxamide | | | | Hz, 1H), 4.16-4.06 (m, 1H), |
| | | | | | 4.00 (d, J = 2.6 Hz, 3H), 2.75 |
| | | | | | (p, J = 7.7 Hz, 1H), 2.55 (s, |
| | | | | | 3H), 1.68 (d, J = 1.2 Hz, 3H), |
| | | | | | 0.79 (dq, J = 7.4, 2.4 Hz, 3H) |
| | | | | | ppm. |

The following compounds were made using a method similar to that described in Example 9, except that different amines were used in step 1 with heating at 40° C. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 325 | (2R,3S,4S,5R)-3- | 517.489 | 518.3 | 3.31 | ¹H NMR (500 MHz, DMSO- |
| | (3,4-difluoro-2- | | | | d₆) δ 9.68 (s, 1H), 7.89 (s, |
| | methoxyphenyl)-4,5- | | | | 1H), 7.21-7.10 (m, 2H), |
| | dimethyl-N-(3- | | | | 5.12 (d, J = 10.6 Hz, 1H), |
| | methyl-1- | | | | 4.28-4.15 (m, 2H), 3.94 (d, |
| | (tetrahydro-2H- | | | | J = 2.0 Hz, 3H), 3.93-3.88 |
| | pyran-4-yl)-1H- | | | | (m, 2H), 3.40 (td, J = 11.2, |
| | pyrazol-4-yl)-5- | | | | 3.5 Hz, 2H), 2.73 (p, J = 7.4 |
| | (trifluoromethyl)- | | | | Hz, 1H), 2.06 (s, 3H), 1.92- |
| | tetrahydrofuran-2- | | | | 1.77 (m, 4H), 1.57 (s, 3H), |
| | carboxamide | | | | 0.76-0.68 (m, 3H) ppm. |
| 326 | (2R,3S,4S,5R)-3- | 517.489 | 518.3 | 3.28 | ¹H NMR (500 MHz, DMSO- |
| | (3,4-difluoro-2- | | | | d₆) δ 9.56 (s, 1H), 7.51 (s, |
| | methoxyphenyl)-4,5- | | | | 1H), 7.23-7.12 (m, 2H), |
| | dimethyl-N-(5- | | | | 5.07 (d, J = 10.6 Hz, 1H), |
| | methyl-1- | | | | 4.30 (tt, J = 11.4, 4.1 Hz, |
| | (tetrahydro-2H- | | | | 1H), 4.18 (dd, J = 10.6, 7.5 |
| | pyran-4-yl)-1H- | | | | Hz, 1H), 3.98-3.90 (m, 5H), |
| | pyrazol-4-yl)-5- | | | | 3.44 (tt, J = 11.8, 2.1 Hz, |
| | (trifluoromethyl)- | | | | 2H), 2.72 (p, J = 7.5 Hz, 1H), |
| | tetrahydrofuran-2- | | | | 2.11 (s, 3H), 2.03-1.89 (m, |
| | carboxamide | | | | 2H), 1.79-1.67 (m, 2H), |
| | | | | | 1.58 (s, 3H), 0.72 (d, J = 7.4 |
| | | | | | Hz, 3H) ppm. |
| 327 | (2R,3S,4S,5R)-3- | 450.423 | 451.1 | 3.48 | ¹H NMR (500 MHz, DMSO- |
| | (3,4-difluoro-2- | | | | d₆) δ 9.96 (s, 1H), 8.95 (s, |
| | methoxyphenyl)-4,5- | | | | 1H), 7.26-7.10 (m, 2H), |
| | dimethyl-N-(3- | | | | 5.23 (d, J = 10.6 Hz, 1H), |
| | methylisothiazol-4- | | | | 4.23 (dd, J = 10.5, 7.5 Hz, |
| | yl)-5- | | | | 1H), 3.94 (d, J = 2.1 Hz, 3H), |
| | (trifluoromethyl)- | | | | 2.76 (p, J = 7.5 Hz, 1H), 2.32 |
| | tetrahydrofuran-2- | | | | (s, 3H), 1.61 (s, 3H), 0.80- |
| | carboxamide | | | | 0.67 (m, 3H) ppm. |
| 328 | (2R,3S,4S,5R)-N- | 470.393 | 470.9 | 3.01 | ¹H NMR (500 MHz, DMSO- |
| | ([1,2,4]triazolo[4,3- | | | | d₆) δ 10.93 (s, 1H), 9.33 (d, |
| | alpyridin-7-yl)-3- | | | | J = 0.8 Hz, 1H), 8.65 (d, J = |
| | (3,4-difluoro-2- | | | | 7.5 Hz, 1H), 8.31-8.25 (m, |
| | methoxyphenyl)-4,5- | | | | 1H), 7.31 (dd, J = 7.5, 1.9 |
| | dimethyl-5- | | | | Hz, 1H), 7.26-7.07 (m, 2H), |
| | (trifluoromethyl)- | | | | 5.15 (d, J = 10.1 Hz, 1H), |
| | tetrahydrofuran-2- | | | | 4.29 (dd, J = 10.1, 7.7 Hz, |
| | carboxamide (TFA | | | | 1H), 3.96 (d, J = 2.0 Hz, 3H), |
| | salt) | | | | 2.79 (p, J = 7.5 Hz, 1H), 1.61 |
| | | | | | (s, 3H), 0.74 (d, J = 7.3 Hz, |
| | | | | | 3H) ppm. |
| 329 | (2R,3S,4S,5R)-N- | 470.393 | 471.2 | 3.29 | ¹H NMR (500 MHz, DMSO- |
| | ([1,2,3]triazolo[1,5- | | | | d₆) δ 10.61 (s, 1H), 9.55 (s, |
| | a]pyridin-6-yl)-3- | | | | 1H), 8.15 (d, J = 1.0 Hz, 1H), |
| | (3,4-difluoro-2- | | | | 7.93 (dd, J = 9.4, 0.9 Hz, |
| | methoxyphenyl)-4,5- | | | | 1H), 7.43 (dd, J = 9.5, 1.7 |
| | dimethyl-5- | | | | Hz, 1H), 7.24-7.12 (m, 2H), |
| | (trifluoromethyl)- | | | | 5.14 (d, J = 10.2 Hz, 1H), |
| | tetrahydrofuran-2- | | | | 4.29 (dd, J = 10.2, 7.7 Hz, |
| | carboxamide | | | | 1H), 3.96 (d, J = 2.0 Hz, 3H), |
| | | | | | 2.79 (p, J = 7.5 Hz, 1H), 1.62 |
| | | | | | (s, 3H), 0.74 (d, J = 7.5 Hz, |
| | | | | | 3H) ppm. |

The following compounds were made using a method similar to that described in Example 9, except that a mixture of 3-methyl-1-(oxetan-3-yl)pyrazol-4-amine and 5-methyl-1-(oxetan-3-yl)pyrazol-4-amine was used in step 1 with heating at 40′° C. The regioisomeric products were separated by chiral SFC using a Chiralpak IC column, Sum particle size, 25 cm×20 mm from Daicel as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 330 | (2R,3S,4S,5R)-3-(3,4- | 489.436 | 490.2 | 3.22 | ¹H NMR (500 MHz, DMSO- |
| | difluoro-2- | | | | d₆) δ 9.75 (s, 1H), 8.01 (s, |
| | methoxyphenyl)-4,5- | | | | 1H), 7.24-7.08 (m, 2H), |
| | dimethyl-N-(3-methyl- | | | | 5.42 (p, J = 7.1 Hz, 1H), 5.13 |
| | 1-(oxetan-3-yl)-1H- | | | | (d, J = 10.6 Hz, 1H), 4.88- |
| | pyrazol-4-yl)-5- | | | | 4.74 (m, 4H), 4.20 (dd, J = |
| | (trifluoromethyl)- | | | | 10.5, 7.4 Hz, 1H), 3.94 (d, J = |
| | tetrahydrofuran-2- | | | | 1.9 Hz, 3H), 2.73 (p, J = |
| | carboxamide | | | | 7.3 Hz, 1H), 2.12 (s, 3H), |
| | (first eluting | | | | 1.57 (s, 3H), 0.72 (d, J = 7.2 |
| | regioisomer by SFC) | | | | Hz, 3H) ppm. |
| 331 | (2R,3S,4S,5R)-3-(3,4- | 489.436 | 490.2 | 3.18 | ¹H NMR (500 MHz, DMSO- |
| | difluoro-2- | | | | d₆) δ 9.64 (s, 1H), 7.65 (s, |
| | methoxyphenyl)-4,5- | | | | 1H), 7.21-7.12 (m, 2H), |
| | dimethyl-N-(5-methyl- | | | | 5.50 (p, J = 7.1 Hz, 1H), 5.07 |
| | 1-(oxetan-3-yl)-1H- | | | | (d, J = 10.5 Hz, 1H), 4.93- |
| | pyrazol-4-yl)-5- | | | | 4.78 (m, 4H), 4.18 (dd, J = |
| | (trifluoromethyl)- | | | | 10.5, 7.5 Hz, 1H), 3.93 (d, J = |
| | tetrahydrofuran-2- | | | | 2.0 Hz, 3H), 2.73 (p, J = |
| | carboxamide | | | | 7.5 Hz, 1H), 2.04 (s, 3H), |
| | (second eluting | | | | 1.58 (s, 3H), 0.72 (d, J = 7.4 |
| | regioisomer by SFC) | | | | Hz, 3H) ppm. |

The following compounds were made using a method similar to that described in Example 9, except that different amines were used in step 1 and General Method B was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 332 | (2R,3S,4S,5R)-3-(3,4- | 507.451 | 508.4 | 2.96 | ¹H NMR (500 MHz, DMSO- |
| | difluoro-2- | | | | d₆) δ 9.68 (s, 1H), 7.85 (s, |
| | methoxyphenyl)-N- | | | | 1H), 7.21-7.11 (m, 2H), |
| | (1-((S)-2,3- | | | | 5.13 (d, J = 10.6 Hz, 1H), |
| | dihydroxypropyl)-3- | | | | 4.84 (d, J = 5.5 Hz, 1H), 4.65 |
| | methyl-1H-pyrazol-4- | | | | (t, J = 5.6 Hz, 1H), 4.21 (dd, |
| | yl)-4,5-dimethyl-5- | | | | J = 10.6, 7.4 Hz, 1H), 4.07- |
| | (trifluoromethyl)- | | | | 4.04 (m, 1H), 3.95 (d, J = 2.0 |
| | tetrahydrofuran-2- | | | | Hz, 3H), 3.81 (dd, J = 13.8, |
| | carboxamide | | | | 7.6 Hz, 1H), 3.73-3.67 (m, |
| | | | | | 1H), 3.30-3.28 (m, 1H), |
| | | | | | 3.24 (dt, J = 11.0, 5.9 Hz, |
| | | | | | 1H), 2.74 (p, J = 7.5 Hz, 1H), |
| | | | | | 2.07 (s, 3H), 1.58 (s, 3H), |
| | | | | | 0.73 (dd, J = 7.8, 2.4 Hz, 3H) |
| | | | | | ppm. |
| 333 | (2R,3S,4S,5R)-3-(3,4- | 504.447 | 505.3 | 3.02 | ¹H NMR (500 MHz, DMSO- |
| | difluoro-2- | | | | d₆) δ 10.53 (s, 1H), 8.34 (d, |
| | methoxyphenyl)-N- | | | | J = 5.7 Hz, 1H), 7.54 (d, J = |
| | (2-((S)-2,3- | | | | 2.1 Hz, 1H), 7.51-7.43 (m, |
| | dihydroxypropyl)- | | | | 1H), 7.21-7.09 (m, 2H), |
| | pyridin-4-yl)-4,5- | | | | 5.09 (d, J = 10.2 Hz, 1H), |
| | dimethyl-5- | | | | 4.76-4.47 (m, 2H), 4.25 (dd, |
| | (trifluoromethyl)- | | | | J = 10.3, 7.6 Hz, 1H), 3.95 |
| | tetrahydrofuran-2- | | | | (d, J = 2.0 Hz, 3H), 3.84- |
| | carboxamide | | | | 3.76 (m, 1H), 2.87 (dd, J = |
| | | | | | 13.6, 4.3 Hz, 1H), 2.77 (p, J = |
| | | | | | 7.6 Hz, 1H), 2.61 (dd, J = |
| | | | | | 13.7, 8.4 Hz, 1H), 1.59 (s, |
| | | | | | 3H), 0.73 (dd, J = 7.4, 2.4 |
| | | | | | Hz, 3H) ppm. |

The following compound was made using a method similar to that described in Example 9, except that a 6-[[(4R)-2,2-dimethyl-1,3-dioxolan-4-yl]methyl]pyridin-3-amine was used in step 1 and diol deprotection was achieved using TFA in THF/water at 60° C. as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 334 | (2R,3S,4S,5R)-3- | 504.447 | 505 | 2.96 | ¹H NMR (500 MHz, DMSO- |
| | (3,4-difluoro-2- | | | | d₆) δ 10.32 (s, 1H), 8.65 (dd, |
| | methoxyphenyl)-N- | | | | J = 2.6, 0.7 Hz, 1H), 7.90 |
| | (6-((R)-2,3- | | | | (dd, J = 8.4, 2.6 Hz, 1H), |
| | dihydroxypropyl)- | | | | 7.25-7.10 (m, 3H), 5.09 (d, |
| | pyridin-3-yl)-4,5- | | | | J = 10.3 Hz, 1H), 4.58 (d, J = |
| | dimethyl-5- | | | | 5.2 Hz, 1H), 4.55 (t, J = 5.7 |
| | (trifluoromethyl)- | | | | Hz, 1H), 4.24 (dd, J = 10.3, |
| | tetrahydrofuran-2- | | | | 7.6 Hz, 1H), 3.95 (d, J = 2.2 |
| | carboxamide | | | | Hz, 3H), 3.86-3.74 (m, 1H), |
| | | | | | 3.37-3.24 (m, 2H), 2.86 (dd, |
| | | | | | J = 13.7, 4.5 Hz, 1H), 2.77 |
| | | | | | (p, J = 7.6 Hz, 1H), 2.63 (dd, |
| | | | | | J = 13.7, 8.2 Hz, 1H), 1.61 (s, |
| | | | | | 3H), 0.74 (d, J = 7.0 Hz, 3H) |
| | | | | | ppm. |

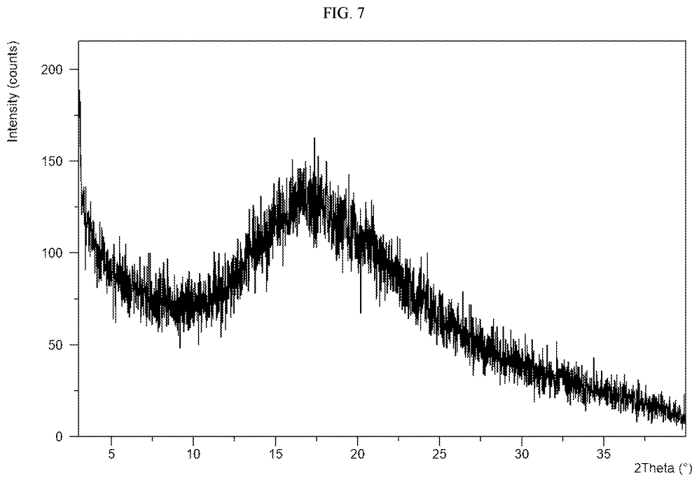

Compound 334 was analyzed by X-ray powder diffraction and determined to be amorphous (see FIG. 7).

The following compounds were made using a method similar to that described in Example 9, except that different amines were used in step 1 and General Method E was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 5 | (2R,3S,4S,5R)-3-(3,4- | 507.451 | 508.4 | 2.96 | ¹H NMR (500 MHz, DMSO- |
| | difluoro-2- | | | | d₆) δ 10.58 (s, 1H), 7.26- |
| | methoxyphenyl)-N- | | | | 7.00 (m, 2H), 6.32 (s, 1H), |
| | (5-((S)-2,3- | | | | 5.06 (d, J = 10.7 Hz, 1H), |
| | dihydroxypropyl)-1- | | | | 4.74 (d, J = 5.2 Hz, 1H), 4.62 |
| | methyl-1H-pyrazol-3- | | | | (t, J = 5.6 Hz, 1H), 4.21 (dd, |
| | yl)-4,5-dimethyl-5- | | | | J = 10.7, 7.5 Hz, 1H), 3.95 |
| | (trifluoromethyl)- | | | | (d, J = 2.0 Hz, 3H), 3.64 (s, |
| | tetrahydrofuran-2- | | | | 3H), 3.26 (td, J = 11.7, 10.8, |
| | carboxamide | | | | 6.3 Hz, 1H), 2.78-2.66 (m, |
| | | | | | 2H), 2.58-2.52 (m, 1H), |
| | | | | | 1.58 (s, 3H), 0.72-0.63 (m, |
| | | | | | 3H) ppm. |
| 335 | (2R,3S,4S,5R)-3-(3,4- | 507.451 | 508.4 | 2.95 | ¹H NMR (500 MHz, DMSO- |
| | difluoro-2- | | | | d₆) δ 10.58 (s, 1H), 7.22- |
| | methoxyphenyl)-N- | | | | 7.12 (m, 2H), 6.32 (s, 1H), |
| | (5-((R)-2,3- | | | | 5.06 (d, J = 10.7 Hz, 1H), |
| | dihydroxypropyl)-1- | | | | 4.73 (d, J = 5.1 Hz, 1H), 4.62 |
| | methyl-1H-pyrazol-3- | | | | (t, J = 5.6 Hz, 1H), 4.21 (dd, |
| | yl)-4,5-dimethyl-5- | | | | J = 10.7, 7.5 Hz, 1H), 3.95 (s, |
| | (trifluoromethyl)- | | | | 3H), 3.64 (s, 4H), 3.30-3.22 |
| | tetrahydrofuran-2- | | | | (m, 1H), 2.81-2.67 (m, 2H), |
| | carboxamide | | | | 2.54 (d, J = 7.7 Hz, 1H), 1.58 |
| | | | | | (s, 3H), 0.70 (d, J = 7.5 Hz, |
| | | | | | 3H) ppm. |

The following compounds were made using a method similar to that described in Example 9, except that 6-(methylthio)pyridin-3-amine was used. General Method G, using IPA instead of MeOH in step 1 and SFC purification using a Chiralpak IC column, Sum particle size, 25 cm×20 mm from Daicel in step 2, was used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 336 | rel- | 507.474 | 508.4 | 3.15 | ¹H NMR (500 MHz, |
| | (2R*,3S*,4S*,5R*)-3- | | | | Methanol-d₄) δ 8.92 (d, J = |
| | (3,4-difluoro-2- | | | | 2.5 Hz, 1H), 8.37 (dd, J = |
| | methoxyphenyl)-4,5- | | | | 8.6, 2.4 Hz, 1H), 8.08 (d, J = |
| | dimethyl-N-(6-(S- | | | | 8.6 Hz, 1H), 7.13 (ddd, J = |
| | methylsulfonimidoyl)- | | | | 8.2, 5.5, 2.1 Hz, 1H), 6.98 |
| | pyridin-3-yl)-5- | | | | (td, J = 9.4, 7.5 Hz, 1H), 5.11 |
| | (trifluoromethyl)- | | | | (d, J = 10.4 Hz, 1H), 4.33 |
| | tetrahydrofuran-2- | | | | (dd, J = 10.4, 8.0 Hz, 1H), |
| | carboxamide | | | | 4.00 (d, J = 2.3 Hz, 3H), 3.21 |
| | (first eluting isomer | | | | (s, 3H), 2.80 (p, J = 7.6 Hz, |
| | by SFC) | | | | 1H), 1.67 (d, J = 1.3 Hz, 3H), |
| | | | | | 0.82 (dq, J = 7.3, 2.3 Hz, 3H) |
| | | | | | ppm. |
| 337 | rel- | 507.474 | 508.3 | 3.15 | ¹H NMR (500 MHz, |
| | (2R*,3S*,4S*,5R*)-3- | | | | Methanol-d₄) δ 8.92 (d, J = |
| | (3,4-difluoro-2- | | | | 2.4 Hz, 1H), 8.37 (dd, J = |
| | methoxyphenyl)-4,5- | | | | 8.6, 2.5 Hz, 1H), 8.08 (d, J = |
| | dimethyl-N-(6-(S- | | | | 8.6 Hz, 1H), 7.13 (ddd, J = |
| | methylsulfonimidoyl)- | | | | 8.2, 5.6, 2.2 Hz, 1H), 6.98 |
| | pyridin-3-yl)-5- | | | | (ddd, J = 9.9, 8.9, 7.5 Hz, |
| | (trifluoromethyl)- | | | | 1H), 5.11 (d, J = 10.4 Hz, |
| | tetrahydrofuran-2- | | | | 1H), 4.33 (dd, J = 10.5, 8.0 |
| | carboxamide | | | | Hz, 1H), 4.00 (d, J = 2.3 Hz, |
| | (second eluting isomer | | | | 3H), 3.21 (s, 3H), 2.80 (p, J = |
| | by SFC) | | | | 7.6 Hz, 1H), 1.67 (d, J = 1.2 |
| | | | | | Hz, 3H), 0.82 (dq, J = 7.5, |
| | | | | | 2.3 Hz, 3H) ppm. |

The following compounds were made using a method similar to that described in Example 9, except that 6-methylsulfanylpyridin-3-amine was used in step 1. The resulting product was oxidized using conditions similar to those described in step 1 of General Method G, then N-methylated using formaldehyde, triethylsilane and TFA and finally purified by chiral SFC using a Lux i-Cellulose-S column, Sum particle size, 25 cm×10 mm from Phenomenex. on a Minigram SFC instrument from Berger Instruments. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 338 | rel- | 521.501 | 523 | 3.26 | ¹H NMR (500 MHz, |
| | (2R*,3S*,4S*,5R*)-3- | | | | Methanol-d₄) δ 8.96 (dd, J = |
| | (3,4-difluoro-2- | | | | 2.6, 0.7 Hz, 1H), 8.39 (dd, J = |
| | methoxyphenyl)-N-(6- | | | | 8.6, 2.5 Hz, 1H), 8.05 (dd, |
| | (N,S- | | | | J = 8.6, 0.7 Hz, 1H), 7.14 |
| | dimethylsulfonimidoyl)- | | | | (ddd, J = 8.2, 5.5, 2.2 Hz, |
| | pyridin-3-yl)-4,5- | | | | 1H), 6.98 (ddd, J = 9.9, 8.9, |
| | dimethyl-5- | | | | 7.5 Hz, 1H), 5.12 (d, J = 10.4 |
| | (trifluoromethyl)- | | | | Hz, 1H), 4.33 (dd, J = 10.4, |
| | tetrahydrofuran-2- | | | | 8.0 Hz, 1H), 4.00 (d, J = 2.3 |
| | carboxamide | | | | Hz, 3H), 3.20 (s, 3H), 2.80 |
| | (first eluting isomer | | | | (p, J = 7.7 Hz, 1H), 2.55 (s, |
| | by SFC) | | | | 3H), 1.71-1.61 (m, 3H), |
| | | | | | 0.82 (dq, J = 7.4, 2.3 Hz, |
| | | | | | 3H). |
| 339 | rel- | 521.501 | 522.9 | 3.26 | ¹H NMR (500 MHz, |
| | (2R*,3S*,4S*,5R*)-3- | | | | Methanol-d₄) δ 8.96 (dd, J = |
| | (3,4-difluoro-2- | | | | 2.5, 0.7 Hz, 1H), 8.39 (dd, J = |
| | methoxyphenyl)-N-(6- | | | | 8.6, 2.5 Hz, 1H), 8.04 (dd, |
| | (N,S- | | | | J = 8.6, 0.7 Hz, 1H), 7.13 |
| | dimethylsulfonimidoyl)- | | | | (ddd, J = 8.3, 5.6, 2.2 Hz, |
| | pyridin-3-yl)-4,5- | | | | 1H), 6.98 (ddd, J = 10.0, 8.9, |
| | dimethyl-5- | | | | 7.5 Hz, 1H), 5.11 (d, J = 10.4 |
| | (trifluoromethyl)- | | | | Hz, 1H), 4.33 (dd, J = 10.4, |
| | tetrahydrofuran-2- | | | | 8.0 Hz, 1H), 4.00 (d, J = 2.2 |
| | carboxamide | | | | Hz, 3H), 3.21 (s, 3H), 2.80 |
| | (second eluting isomer | | | | (p, J = 7.6 Hz, 1H), 2.55 (s, |
| | by SFC) | | | | 3H), 1.69-1.65 (m, 3H), |
| | | | | | 0.82 (dq, J = 7.4, 2.3 Hz, |
| | | | | | 3H). |

The following compounds were made using a method similar to that described in Example 9, except that different amines were used in step 1 and General Method I was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 340 | (2R,3S,4S,5R)-N-(2- | 501.446 | 502 | 3.07 | ¹H NMR (400 MHz, DMSO- |
| | (3-aminooxetan-3- | | | | d₆) δ 10.60 (s, 1H), 8.46 (dd, |
| | yl)pyridin-4-yl)-3- | | | | J = 5.5, 0.6 Hz, 1H), 7.89 |
| | (3,4-difluoro-2- | | | | (dd, J = 2.1, 0.7 Hz, 1H), |
| | methoxyphenyl)-4,5- | | | | 7.58 (dd, J = 5.6, 2.0 Hz, |
| | dimethyl-5- | | | | 1H), 7.24-7.08 (m, 2H), |
| | (trifluoromethyl)- | | | | 5.10 (d, J = 10.3 Hz, 1H), |
| | tetrahydrofuran-2- | | | | 4.83 (dd, J = 5.5, 4.4 Hz, |
| | carboxamide | | | | 2H), 4.54 (d, J = 5.6 Hz, 2H), |
| | | | | | 4.25 (dd, J = 10.4, 7.7 Hz, |
| | | | | | 1H), 3.96 (d, J = 2.3 Hz, 3H), |
| | | | | | 2.78 (p, J = 7.5 Hz, IH), 2.65 |
| | | | | | (s, 2H), 1.60 (s, 3H), 0.76- |
| | | | | | 0.69 (m, 3H) ppm. |
| 341 | (2R,3S,4S,5R)-N-(2-(3- | 519.437 | 520.4 | 3.28 | ¹H NMR (500 MHz, DMSO- |
| | aminooxetan-3-yl)-5- | | | | d₆) δ 10.30 (s, 1H), 8.55 (d, |
| | fluoropyridin-4-yl)-3- | | | | J = 2.2 Hz, 1H), 8.36 (d, J = |
| | (3,4-difluoro-2- | | | | 6.3 Hz, 1H), 7.22-7.15 (m, |
| | methoxyphenyl)-4,5- | | | | 2H), 5.35 (d, J = 10.4 Hz, |
| | dimethyl-5- | | | | 1H), 4.82 (d, J = 5.6 Hz, 2H), |
| | (trifluoromethyl)- | | | | 4.52 (dd, J = 5.6, 3.4 Hz, |
| | tetrahydrofuran-2- | | | | 2H), 4.25 (dd, J = 10.4, 7.5 |
| | carboxamide | | | | Hz, 1H), 3.96 (d, J = 2.0 Hz, |
| | | | | | 3H), 2.78 (dq, J = 7.5, 7.5 |
| | | | | | Hz, 1H), 2.65 (s, 2H), 1.61 |
| | | | | | (s, 3H), 0.73 (d, J = 6.4 Hz, |
| | | | | | 3H) ppm. |

The following compounds were made using a method similar to that described in Example 9, except that different chiral amines were used in step 1 and SFC purification was run after step 1 to separate the diastereomers generated. General method I was used on separated isomers as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 342 | rel- | 515.473 | 516.4 | 2.83 | ¹H NMR (500 MHz, DMSO- |
| | (2R*,3S*,4S*,5R*)-3- | | | | d₆) δ 10.55 (s, 1H), 8.35 (d, J |
| | (3,4-difluoro-2- | | | | = 5.6 Hz, 1H), 7.76 (d, J = |
| | methoxyphenyl)-4,5- | | | | 2.1 Hz, 1H), 7.51 (dd, J = |
| | dimethyl-N-(2- | | | | 5.5, 2.1 Hz, 1H), 7.21-7.09 |
| | (morpholin-3- | | | | (m, 2H), 5.08 (d, J = 10.3 Hz, |
| | yl)pyridin-4-yl)-5- | | | | 1H), 4.24 (dd, J = 10.3, 7.7 |
| | (trifluoromethyl)- | | | | Hz, 1H), 3.95 (d, J = 2.0 Hz, |
| | tetrahydrofuran-2- | | | | 3H), 3.87-3.77 (m, 2H), |
| | carboxamide | | | | 3.72 (d, J = 10.8 Hz, 1H), |
| | (precursor was first | | | | 3.44-3.37 (m, 1H), 3.19 (t, |
| | eluting isomer by SFC | | | | J = 9.7 Hz, 1H), 2.86 (d, J = |
| | using Lux i-Cellulose- | | | | 5.9 Hz, 2H), 2.77 (p, J = 7.6 |
| | 5 column) | | | | Hz, 1H), 1.60 (s, 3H), 0.77- |
| | | | | | 0.67 (m, 3H) ppm. |
| 343 | rel- | 515.473 | 516.4 | 2.84 | ¹H NMR (500 MHz, DMSO- |
| | (2R*,3S*,4S*,5R*)-3- | | | | d₆) δ 10.54 (s, 1H), 8.35 (d, |
| | (3,4-difluoro-2- | | | | J = 5.6 Hz, 1H), 7.76 (d, J = |
| | methoxyphenyl)-4,5- | | | | 2.1 Hz, 1H), 7.51 (dd, J = |
| | dimethyl-N-(2- | | | | 5.6, 2.1 Hz, 1H), 7.23-7.08 |
| | (morpholin-3- | | | | (m, 2H), 5.08 (d, J = 10.2 Hz, |
| | yl)pyridin-4-yl)-5- | | | | 1H), 4.24 (dd, J = 10.4, 7.7 |
| | (trifluoromethyl)- | | | | Hz, 1H), 3.95 (d, J = 2.1 Hz, |
| | tetrahydrofuran-2- | | | | 3H), 3.87-3.78 (m, 2H), |
| | carboxamide | | | | 3.72 (d, J = 10.8 Hz, 1H), |
| | (precursor was second | | | | 3.45-3.37 (m, 1H), 3.23- |
| | eluting isomer by SFC | | | | 3.15 (m, 1H), 2.86 (d, J = 6.6 |
| | using Lux i-Cellulose- | | | | Hz, 2H), 2.77 (p, J = 7.5 Hz, |
| | 5 column) | | | | 1H), 1.60 (s, 3H), 0.75-0.69 |
| | | | | | (m, 3H) ppm. |
| 344 | rel- | 545.542 | 545.8 | 4.08 | ¹H NMR (500 MHz, DMSO- |
| | (2R*,3S*,4S*,5R*)-3- | | | | d₆) δ 10.59 (s, 1H), 8.35 (d, |
| | (3,4-difluoro-2- | | | | J = 5.5 Hz, 1H), 7.64 (d, J = |
| | methoxyphenyl)-N-(2- | | | | 2.1 Hz, 1H), 7.54 (dd, J = |
| | (2-methoxy-2-methyl- | | | | 5.5, 2.1 Hz, 1H), 7.20-7.09 |
| | 1- | | | | (m, 2H), 5.08 (d, J = 10.3 Hz, |
| | (methylamino)propyl) | | | | 1H), 4.25 (dd, J = 10.3, 7.7 |
| | pyridin-4-yl)-4,5- | | | | Hz, 1H), 3.95 (d, J = 2.0 Hz, |
| | dimethyl-5- | | | | 3H), 3.55 (s, 1H), 3.13 (s, |
| | (trifluoromethyl)- | | | | 3H), 2.80-2.73 (m, 1H), |
| | tetrahydrofuran-2- | | | | 2.09 (s, 3H), 1.59 (s, 3H), |
| | carboxamide | | | | 1.03 (s, 3H), 0.97 (s, 3H), |
| | (precursor was first | | | | 0.72 (d, J = 7.5 Hz, 3H) ppm. |
| | eluting isomer by SFC | | | | |
| | using Lux Cellulose-2 | | | | |
| | column) | | | | |
| 345 | rel- | 545.542 | 547 | 4.12 | ¹H NMR (500 MHz, DMSO- |
| | (2R*,3S*,4S*,5R*)-3- | | | | d₆) δ 10.59 (s, 1H), 8.35 (d, |
| | (3,4-difluoro-2- | | | | J = 5.5 Hz, 1H), 7.64 (d, J = |
| | methoxyphenyl)-N-(2- | | | | 2.1 Hz, 1H), 7.54 (dd, J = |
| | (2-methoxy-2-methyl-1- | | | | 5.5, 2.1 Hz, 1H), 7.20-7.09 |
| | (methylamino)propyl) | | | | (m, 2H), 5.08 (d, J = 10.3 Hz, |
| | pyridin-4-yl)-4,5- | | | | 1H), 4.25 (dd, J = 10.3, 7.7 |
| | dimethyl-5- | | | | Hz, 1H), 3.95 (d, J = 2.0 Hz, |
| | (trifluoromethyl)- | | | | 3H), 3.55 (s, 1H), 3.13 (s, |
| | tetrahydrofuran-2- | | | | 3H), 2.80-2.73 (m, 1H), |
| | carboxamide | | | | 2.09 (s, 3H), 1.59 (s, 3H), |
| | (precursor was second | | | | 1.03 (s, 3H), 0.97 (s, 3H), |
| | eluting isomer by SFC | | | | 0.72 (d, J = 7.5 Hz, 3H) ppm. |
| | using Lux Cellulose-2 | | | | |
| | column) | | | | |
| 346 | rel- | 515.473 | 516 | 3.12 | ¹H NMR (500 MHz, DMSO- |
| | (2R*,3S*,4S*,5R*)-N- | | | | d₆) δ 10.57 (s, 1H), 8.39 (d, |
| | (2-(3- | | | | J = 5.5 Hz, 1H), 7.90 (dd, J = |
| | aminotetrahydrofuran- | | | | 2.1, 0.7 Hz, 1H), 7.53 (dd, |
| | 3-yl)pyridin-4-yl)-3- | | | | J = 5.5, 2.0 Hz, 1H), 7.24- |
| | (3,4-difluoro-2- | | | | 7.08 (m, 2H), 5.10 (d, J = |
| | methoxyphenyl)-4,5- | | | | 10.3 Hz, 1H), 4.25 (dd, J = |
| | dimethyl-5- | | | | 10.3, 7.6 Hz, 1H), 4.02 (td, |
| | (trifluoromethyl)- | | | | J = 8.3, 7.0 Hz, 1H), 3.96 (d, |
| | tetrahydrofuran-2- | | | | J = 2.2 Hz, 3H), 3.95-3.90 |
| | carboxamide | | | | (m, 1H), 3.88 (d, J = 8.1 Hz, |
| | (precursor was first | | | | 1H), 3.63 (dd, J = 8.2, 0.8 |
| | eluting isomer by SFC | | | | Hz, 1H), 2.78 (p, J = 7.4 Hz, |
| | using Chiralpak IG | | | | 1H), 2.42 (dt, J = 12.1, 8.7 |
| | column) | | | | Hz, 1H), 2.28 (s, 2H), 1.92 |
| | | | | | (dddd, J = 11.9, 7.0, 3.9, 0.9 |
| | | | | | Hz, 1H), 1.60 (s, 3H), 0.73 |
| | | | | | (dd, J = 7.0, 2.5 Hz, 3H) |
| | | | | | ppm. |
| 347 | rel- | 515.473 | 516 | 3.12 | ¹H NMR (500 MHz, DMSO- |
| | (2R*,3S*,4S*,5R*)-N- | | | | d₆) δ 10.57 (s, 1H), 8.39 (d, |
| | (2-(3- | | | | J = 5.5 Hz, 1H), 7.90 (dd, J = |
| | aminotetrahydrofuran- | | | | 2.1, 0.7 Hz, 1H), 7.53 (dd, |
| | 3-yl)pyridin-4-yl)-3- | | | | J = 5.5, 2.0 Hz, 1H), 7.24- |
| | (3,4-difluoro-2- | | | | 7.08 (m, 2H), 5.10 (d, J = |
| | methoxyphenyl)-4,5- | | | | 10.3 Hz, 1H), 4.25 (dd, J = |
| | dimethyl-5- | | | | 10.3, 7.6 Hz, 1H), 4.08-3.99 |
| | (trifluoromethyl)- | | | | (m, 1H), 3.96 (d, J = 2.2 Hz, |
| | tetrahydrofuran-2- | | | | 3H), 3.95-3.86 (m, 2H), |
| | carboxamide | | | | 3.63 (dd, J = 8.1, 0.8 Hz, |
| | (precursor was second | | | | 1H), 2.78 (p, J = 7.5 Hz, 1H), |
| | eluting isomer by SFC | | | | 2.42 (dt, J = 12.1, 8.7 Hz, |
| | using Chiralpak IG | | | | 1H), 2.33 (s, 2H), 1.92 |
| | column) | | | | (dddd, J = 12.0, 7.0, 3.9, 0.8 |
| | | | | | Hz, 1H), 1.60 (s, 3H), 0.77- |
| | | | | | 0.70 (m, 3H) ppm. |
| 348 | rel- | 533.463 | 534.1 | 3.32 | ¹H NMR (500 MHz, DMSO- |
| | (2R*,3S*,4S*,5R*)-N- | | | | d₆) δ 10.26 (s, 1H), 8.47 (d, |
| | (2-(3- | | | | J = 2.1 Hz, 1H), 8.39 (d, J = |
| | aminotetrahydrofuran- | | | | 6.3 Hz, 1H), 7.21-7.14 (m, |
| | 3-yl)-5-fluoropyridin- | | | | 2H), 5.34 (d, J = 10.5 Hz, |
| | 4-yl)-3-(3,4-difluoro- | | | | 1H), 4.24 (dd, J = 10.5, 7.5 |
| | 2-methoxyphenyl)-4,5- | | | | Hz, 1H), 4.02-3.97 (m, 1H), |
| | dimethyl-5- | | | | 3.95 (d, J = 2.0 Hz, 3H), 3.89 |
| | (trifluoromethyl)- | | | | (td, J = 8.4, 3.9 Hz, 1H), 3.85 |
| | tetrahydrofuran-2- | | | | (d, J = 8.1 Hz, 1H), 3.60 (d, |
| | carboxamide | | | | J = 8.1 Hz, 1H), 2.77 (dq, J = |
| | (precursor was first | | | | 7.5, 7.5 Hz, 1H), 2.39 (dt, J = |
| | eluting isomer by SFC | | | | 12.1, 8.7 Hz, 1H), 2.11 (s, |
| | using Chiralpak IC | | | | 2H), 1.93-1.87 (m, 1H), |
| | column) | | | | 1.60 (s, 3H), 0.72 (d, J = 6.4 |
| | | | | | Hz, 3H) ppm. |
| 349 | rel- | 533.463 | 534.1 | 3.32 | ¹H NMR (500 MHz, DMSO- |
| | (2R*,3S*,4S*,5R*)-N- | | | | d₆) δ 10.26 (s, 1H), 8.47 (d, |
| | (2-(3- | | | | J = 2.1 Hz, 1H), 8.39 (d, J = |
| | aminotetrahydrofuran- | | | | 6.3 Hz, 1H), 7.21-7.14 (m, |
| | 3-yl)-5-fluoropyridin- | | | | 2H), 5.34 (d, J = 10.5 Hz, |
| | 4-yl)-3-(3,4-difluoro- | | | | 1H), 4.24 (dd, J = 10.5, 7.5 |
| | 2-methoxyphenyl)-4,5- | | | | Hz, 1H), 4.02-3.97 (m, 1H), |
| | dimethyl-5- | | | | 3.95 (d, J = 2.0 Hz, 3H), 3.89 |
| | (trifluoromethyl)- | | | | (td, J = 8.4, 3.9 Hz, 1H), 3.85 |
| | tetrahydrofuran-2- | | | | (d, J = 8.1 Hz, 1H), 3.60 (d, |
| | carboxamide | | | | J = 8.1 Hz, 1H), 2.77 (dq, J = |
| | (precursor was second | | | | 7.5, 7.5 Hz, 1H), 2.39 (dt, J = |
| | eluting isomer by SFC | | | | 12.1, 8.7 Hz, 1H), 2.11 (s, |
| | using Chiralpak IC | | | | 2H), 1.93-1.87 (m, 1H), |
| | column) | | | | 1.60 (s, 3H), 0.72 (d, J = 6.4 |
| | | | | | Hz, 3H) ppm. |
| 350 | rel- | 533.463 | 534.1 | 3.35 | ¹H NMR (500 MHz, DMSO- |
| | (2R*,3S*,4S*,5R*)-3- | | | | d₆) δ 10.25 (s, 1H), 8.46 (d, |
| | (3,4-difluoro-2- | | | | J = 2.3 Hz, 1H), 8.15 (d, J = |
| | methoxyphenyl)-N-(5- | | | | 6.5 Hz, 1H), 7.20-7.15 (m, |
| | fluoro-2-(morpholin-2- | | | | 2H), 5.34 (d, J = 10.4 Hz, |
| | yl)pyridin-4-yl)-4,5- | | | | 1H), 4.36 (dd, J = 10.2, 2.6 |
| | dimethyl-5- | | | | Hz, 1H), 4.24 (dd, J = 10.4, |
| | (trifluoromethyl)- | | | | 7.5 Hz, 1H), 3.95 (d, J = 2.0 |
| | tetrahydrofuran-2- | | | | Hz, 3H), 3.89-3.85 (m, 1H), |
| | carboxamide | | | | 3.59 (td, J = 11.1, 3.1 Hz, |
| | (precursor was first | | | | 1H), 3.07 (dd, J = 12.3, 2.6 |
| | eluting isomer by SFC | | | | Hz, 1H), 2.78 (dq, J = 7.5, |
| | using Chiralpak IC | | | | 7.5 Hz, 1H), 2.74-2.63 (m, |
| | column) | | | | 2H), 2.41 (t, J = 12.3, 10.2 |
| | | | | | Hz, 1H), 1.60 (s, 3H), 0.72 |
| | | | | | (d, J = 6.4 Hz, 3H) ppm. |
| 351 | rel | 533.463 | 534.1 | 3.35 | ¹H NMR (500 MHz, DMSO- |
| | (2R*,3S*,4S*,5R*)-3- | | | | d₆) δ 10.26 (s, 1H), 8.47 (d, |
| | (3,4-difluoro-2- | | | | J = 2.3 Hz, 1H), 8.16 (d, J = |
| | methoxyphenyl)-N-(5- | | | | 6.4 Hz, 1H), 7.21-7.15 (m, |
| | fluoro-2-(morpholin-2- | | | | 2H), 5.35 (d, J = 10.4 Hz, |
| | yl)pyridin-4-yl)-4,5- | | | | 1H), 4.36 (dd, J = 10.1, 2.7 |
| | dimethyl-5- | | | | Hz, 1H), 4.25 (dd, J = 10.4, |
| | (trifluoromethyl)- | | | | 7.5 Hz, 1H), 3.96 (d, J = 2.0 |
| | tetrahydrofuran-2- | | | | Hz, 3H), 3.90-3.86 (m, 1H), |
| | carboxamide | | | | 3.59 (td, J = 10.9, 3.3 Hz, |
| | (precursor was second | | | | 1H), 3.09 (dd, J = 12.3, 2.6 |
| | eluting isomer by SFC | | | | Hz, 1H), 2.78 (dq, J = 7.5, |
| | using Chiralpak IC | | | | 7.5 Hz, 1H), 2.76-2.63 (m, |
| | column) | | | | 2H), 2.43 (t, J = 12.3, 10.1 |
| | | | | | Hz, 1H), 1.61 (s, 3H), 0.73 |
| | | | | | (d, J = 6.3 Hz, 3H) ppm. |
| 352 | rel- | 517.489 | 518.4 | 3.36 | ¹H NMR (500 MHz, DMSO- |
| | (2R*,3S*,4S*,5R*)-N- | | | | d₆) δ 10.51 (s, 1H), 8.37 (d, |
| | (2-(2-amino-1- | | | | J = 5.5 Hz, 1H), 7.81 (d, J = |
| | methoxypropan-2- | | | | 2.0 Hz, 1H), 7.50 (dd, J = |
| | yl)pyridin-4-yl)-3- | | | | 5.5, 2.0 Hz, 1H), 7.21-7.15 |
| | (3,4-difluoro-2- | | | | (m, 1H), 7.14-7.10 (m, 1H), |
| | methoxyphenyl)-4,5- | | | | 5.08 (d, J = 10.4 Hz, 1H), |
| | dimethyl-5- | | | | 4.25 (dd, J = 10.4, 7.5 Hz, |
| | (trifluoromethyl)- | | | | 1H), 3.95 (d, J = 2.0 Hz, 3H), |
| | tetrahydrofuran-2- | | | | 3.55 (d, J = 8.5 Hz, 1H), 3.37 |
| | carboxamide | | | | (d, J = 8.5 Hz, 1H), 3.16 (s, |
| | (precursor was first | | | | 3H), 2.77 (dq, J = 7.5, 7.5 |
| | eluting isomer by SFC | | | | Hz, 1H), 2.01 (s, 2H), 1.60 |
| | using Chiralpak IG | | | | (s, 3H), 1.27 (s, 3H), 0.72 (d, |
| | column) | | | | J = 6.3 Hz, 3H) ppm. |
| 353 | rel- | 517.489 | 518.4 | 3.36 | ¹H NMR (500 MHz, DMSO- |
| | (2R*,3S*,4S*,5R*)-N- | | | | d₆) δ 10.52 (s, 1H), 8.37 (d, |
| | (2-(2-amino-1- | | | | J = 5.5 Hz, 1H), 7.81 (d, J = |
| | methoxypropan-2- | | | | 2.0 Hz, 1H), 7.50 (dd, J = |
| | yl)pyridin-4-yl)-3- | | | | 5.5, 2.0 Hz, 1H), 7.20-7.15 |
| | (3,4-difluoro-2- | | | | (m, 1H), 7.15-7.10 (m, 1H), |
| | methoxyphenyl)-4,5- | | | | 5.08 (d, J = 10.3 Hz, 1H), |
| | dimethyl-5- | | | | 4.25 (dd, J = 10.3, 7.5 Hz, |
| | (trifluoromethyl)- | | | | 1H), 3.95 (d, J = 2.1 Hz, 3H), |
| | tetrahydrofuran-2- | | | | 3.55 (d, J = 8.5 Hz, 1H), 3.37 |
| | carboxamide | | | | (d, J = 8.5 Hz, 1H), 3.16 (s, |
| | (precursor was second | | | | 3H), 2.77 (dq, J = 7.5, 7.5 |
| | eluting isomer by SFC | | | | Hz, 1H), 1.88 (s, 2H), 1.60 |
| | using Chiralpak IG | | | | (s, 3H), 1.27 (s, 3H), 0.72 (d, |
| | column) | | | | J = 6.4 Hz, 3H) ppm. |
| 354 | rel- | 535.479 | 536.5 | 3.52 | ¹H NMR (500 MHz, DMSO- |
| | (2R*,3S*,4S*,5R*)-N- | | | | d₆) δ 10.21 (s, 1H), 8.45 (d, |
| | (2-(2-amino-1- | | | | J = 2.1 Hz, 1H), 8.30 (d, J = |
| | methoxypropan-2-yl)- | | | | 6.4 Hz, 1H), 7.21-7.14 (m, |
| | 5-fluoropyridin-4-yl)- | | | | 2H), 5.32 (d, J = 10.4 Hz, |
| | 3-(3,4-difluoro-2- | | | | 1H), 4.24 (dd, J = 10.4, 7.5 |
| | methoxyphenyl)-4,5- | | | | Hz, 1H), 3.95 (d, J = 2.0 Hz, |
| | dimethyl-5- | | | | 3H), 3.54 (d, J = 8.5 Hz, 1H), |
| | (trifluoromethyl)- | | | | 3.34 (d, J = 8.5 Hz, 1H), 3.15 |
| | tetrahydrofuran-2- | | | | (s, 3H), 2.76 (dq, J = 7.5, 7.5 |
| | carboxamide | | | | Hz, 1H), 1.90 (s, 2H), 1.60 |
| | (precursor was first | | | | (s, 3H), 1.25 (s, 3H), 0.72 (d, |
| | eluting isomer by SFC | | | | J = 6.5 Hz, 3H) ppm. |
| | using Lux i-Cellulose- | | | | |
| | 5 column) | | | | |
| 355 | rel- | 535.479 | 536.5 | 3.52 | ¹H NMR (500 MHz, DMSO- |
| | (2R*,3S*,4S*,5R*)-N- | | | | d₆) δ 10.21 (s, 1H), 8.45 (d, |
| | (2-(2-amino-1- | | | | J = 2.1 Hz, 1H), 8.31 (d, J = |
| | methoxypropan-2-yl)- | | | | 6.4 Hz, 1H), 7.22-7.14 (m, |
| | 5-fluoropyridin-4-yl)- | | | | 2H), 5.32 (d, J = 10.5 Hz, |
| | 3-(3,4-difluoro-2- | | | | 1H), 4.24 (dd, J = 10.5, 7.5 |
| | methoxyphenyl)-4,5- | | | | Hz, 1H), 3.95 (d, J = 2.0 Hz, |
| | dimethyl-5- | | | | 3H), 3.54 (d, J = 8.5 Hz, 1H), |
| | (trifluoromethyl)- | | | | 3.34 (d, J = 8.5 Hz, 1H), 3.15 |
| | tetrahydrofuran-2- | | | | (s, 3H), 2.77 (dq, J = 7.5, 7.5 |
| | carboxamide | | | | Hz, 1H), 1.89 (s, 2H), 1.61 |
| | (precursor was second | | | | (s, 3H), 1.25 (s, 3H), 0.72 (d, |
| | eluting isomer by SFC | | | | J = 6.4 Hz, 3H) ppm. |
| | using Lux i-Cellulose- | | | | |
| | 5 column) | | | | |
| 356 | rel- | 549.506 | 550.7 | 3.6 | ¹H NMR (500 MHz, DMSO- |
| | (2R*,3S*,4S*,5R*)-N- | | | | d₆) δ 10.21 (s, 1H), 8.43 (s, |
| | (2-(1-amino-2- | | | | 1H), 8.07 (d, J = 6.4 Hz, 1H), |
| | methoxy-2- | | | | 7.21-7.15 (m, 2H), 5.31 (d, |
| | methylpropyl)-5- | | | | J = 10.4 Hz, 1H), 4.24 (dd, J = |
| | fluoropyridin-4-yl)-3- | | | | 10.4, 7.6 Hz, 1H), 3.95 (d, |
| | (3,4-difluoro-2- | | | | J = 2.0 Hz, 3H), 3.88 (s, 1H), |
| | methoxyphenyl)-4,5- | | | | 3.11 (s, 3H), 2.76 (p, J = 7.5 |
| | dimethyl-5- | | | | Hz, 1H), 1.90 (s, 2H), 1.60 |
| | (trifluoromethyl)- | | | | (s, 3H), 1.01 (s, 3H), 0.97 (s, |
| | tetrahydrofuran-2- | | | | 3H), 0.75-0.69 (m, 3H). |
| | carboxamide | | | | |
| | (precursor was first | | | | |
| | eluting isomer by SFC | | | | |
| | using Whelk-O1 | | | | |
| | column) | | | | |
| 357 | rel- | 549.506 | 550.7 | 3.61 | ¹H NMR (500 MHz, DMSO- |
| | (2R*,3S*,4S*,5R*)-N- | | | | d₆) δ 10.23 (s, 1H), 8.42 (s, |
| | (2-(1-amino-2- | | | | 1H), 8.08 (d, J = 6.5 Hz, 1H), |
| | methoxy-2- | | | | 7.23-7.13 (m, 2H), 5.30 (d, |
| | methylpropyl)-5- | | | | J = 10.4 Hz, 1H), 4.23 (dd, |
| | fluoropyridin-4-yl)-3- | | | | J = 10.5, 7.5 Hz, 1H), 3.95 (d, |
| | (3,4-difluoro-2- | | | | J = 2.0 Hz, 3H), 3.89 (s, 1H), |
| | methoxyphenyl)-4,5- | | | | 3.11 (s, 3H), 2.76 (p, J = 7.5 |
| | dimethyl-5- | | | | Hz, 1H), 1.90 (s, 2H), 1.60 |
| | (trifluoromethyl)- | | | | (s, 3H), 1.01 (s, 3H), 0.96 (s, |
| | tetrahydrofuran-2- | | | | 3H), 0.74-0.70 (m, 3H). |
| | carboxamide | | | | |
| | (precursor was second | | | | |
| | eluting isomer by SFC | | | | |
| | using Whelk-O1 | | | | |
| | column) | | | | |

The following compounds were made using a method similar to that described in Example 9, except that tert-butyl N-[1-(4-amino-2-pyridyl)-2-[tert-butyl(dimethyl)silyl]oxy-1-methyl-ethyl]carbamate was used in step 1 and the isomers generated were purified by chiral SFC using a (R,R)-Whelk-O1 column, 5 μm particle size, 25 cm×21.2 mm from Regis Technologies. Boc and TBS deprotection using General method I with 10 vol % water was used on the separated isomers as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 358 | rel-(2R*,3S*,4S*,5R*)- | 503.462 | 504.6 | 3.16 | ¹H NMR (500 MHz, DMSO- |
| | N-(2-(2-amino-1- | | | | d₆) δ 10.53 (s, 1H), 8.36 (d, |
| | hydroxypropan-2- | | | | J = 5.5 Hz, 1H), 7.81 (d, J = |
| | yl)pyridin-4-yl)-3-(3,4- | | | | 2.0 Hz, 1H), 7.50 (dd, J = |
| | difluoro-2- | | | | 5.5, 2.0 Hz, 1H), 7.21-7.09 |
| | methoxyphenyl)-4,5- | | | | (m, 2H), 5.09 (d, J = 10.3 Hz, |
| | dimethyl-5- | | | | 1H), 4.65 (t, J = 5.4 Hz, 1H), |
| | (trifluoromethyl)- | | | | 4.25 (dd, J = 10.3, 7.5 Hz, |
| | tetrahydrofuran-2- | | | | 1H), 3.95 (d, J = 2.1 Hz, 3H), |
| | carboxamide | | | | 3.53 (dd, J = 10.1, 5.4 Hz, |
| | (precursor was first | | | | 1H), 3.41 (dd, J = 10.1, 5.0 |
| | eluting isomer by SFC | | | | Hz, 1H), 2.77 (dq, J = 7.5, |
| | using Whelk-O1 | | | | 7.5 Hz, 1H), 1.85 (s, 2H), |
| | column) | | | | 1.60 (s, 3H), 1.25 (s, 3H), |
| | | | | | 0.72 (d, J = 6.2 Hz, 3H) ppm. |
| 359 | rel-(2R*,3S*,4S*,5R*)- | 503.462 | 504.6 | 3.16 | ¹H NMR (500 MHz, DMSO- |
| | N-(2-(2-amino-1- | | | | d₆) δ 10.52 (s, 1H), 8.36 (d, J |
| | hydroxypropan-2- | | | | = 5.5 Hz, 1H), 7.81 (d, J = |
| | yl)pyridin-4-yl)-3-(3,4- | | | | 2.0 Hz, 1H), 7.51 (dd, J = |
| | difluoro-2- | | | | 5.5, 2.0 Hz, 1H), 7.20-7.09 |
| | methoxyphenyl)-4,5- | | | | (m, 2H), 5.09 (d, J = 10.3 Hz, |
| | dimethyl-5- | | | | 1H), 4.66 (t, J = 5.5 Hz, 1H), |
| | (trifluoromethyl)tetrahy | | | | 4.25 (dd, J = 10.3, 7.5 Hz, |
| | drofuran-2-carboxamide | | | | 1H), 3.95 (d, J = 2.1 Hz, 3H), |
| | (precursor was second | | | | 3.53 (dd, J = 10.1, 5.5 Hz, |
| | eluting isomer by SFC | | | | 1H), 3.42 (dd, J = 10.1, 5.2 |
| | using Whelk-O1 | | | | Hz, 1H), 2.77 (dq, J = 7.5, |
| | column) | | | | 7.5 Hz, 1H), 1.85 (s, 2H), |
| | | | | | 1.60 (s, 3H), 1.25 (s, 3H), |
| | | | | | 0.72 (d, J = 6.3 Hz, 3H) ppm. |

The following compound was made using a method similar to that described in Example 9, except that tert-butyl N-[1-(4-amino-S-fluoro-2-pyridyl)-2-[tert-butyl(dimethyl)silyl]oxy-1-methyl-ethyl]carbamate was used in step 1. Boc and TBS deprotection using General method I with 10 vol % water was used on the epimeric mixture as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 360 | (2R,3S,4S,5R)-N-(2-(2- | 521.453 | 522.6 | 3.29 | ¹H NMR (500 MHz, DMSO- |
| | amino-1- | | | | d₆) δ 10.20 (s, 1H), 8.45 (d, J = |
| | hydroxypropan-2-yl)-5- | | | | 2.1 Hz, 1H), 8.32 (dd, J = |
| | fluoropyridin-4-yl)-3- | | | | 6.4, 1.5 Hz, 1H), 7.22-7.14 |
| | (3,4-difluoro-2- | | | | (m, 2H), 5.32 (d, J = 10.5 Hz, |
| | methoxyphenyl)-4,5- | | | | 1H), 4.64-4.60 (m, 1H), |
| | dimethyl-5- | | | | 4.24 (dd, J = 10.5, 7.5 Hz, |
| | (trifluoromethyl) | | | | 1H), 3.95 (d, J = 2.0 Hz, 3H), |
| | tetrahydrofuran-2- | | | | 3.53 (dd, J = 10.1, 5.5 Hz, |
| | carboxamide, as a | | | | 1H), 3.39 (dd, J = 10.1, 5.3 |
| | mixture of epimers at | | | | Hz, 1H), 2.77 (dq, J = 7.5, |
| | the 2-amino-1- | | | | 7.5 Hz, 1H), 1.61 (s, 3H), |
| | hydroxypropan-2-yl | | | | 1.23 (d, J = 1.7 Hz, 3H), 0.72 |
| | group | | | | (d, J = 6.2 Hz, 3H) ppm. |

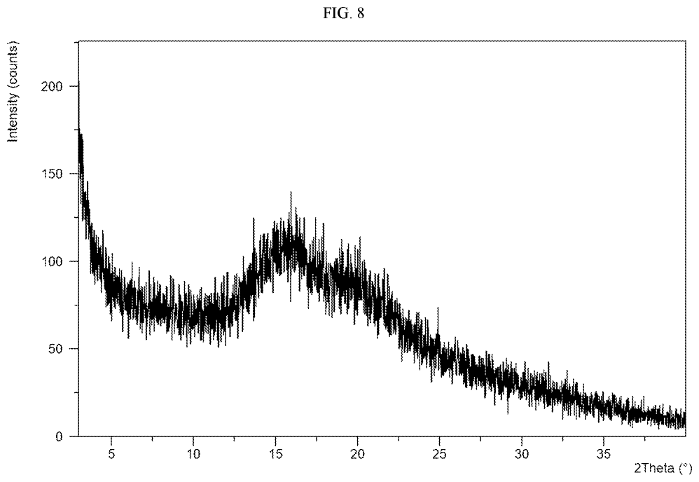

Compound 360 was analyzed by X-ray powder diffraction and determined to be amorphous (see FIG. 8).

The following compounds were made using a method similar to that described in Example 9, except that different amines were used in step 1. Products from step 1 were deprotected using General Method I and then methylated via reductive amination using General Method K as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 361 | (2R,3S,4S,5R)-3-(3,4- | 515.473 | 516 | 3.15 | 1¹H NMR (400 MHz, |
| | difluoro-2- | | | | DMSO-d₆) δ 10.59 (s, 1H), |
| | methoxyphenyl)-4,5- | | | | 8.46 (d, J = 5.5 Hz, 1H), 7.75 |
| | dimethyl-N-(2-(3- | | | | (dd, J = 2.1, 0.6 Hz, 1H), |
| | (methylamino)oxetan-3- | | | | 7.55 (dd, J = 5.6, 2.1 Hz, |
| | yl)pyridin-4-yl)-5- | | | | 1H), 7.25-7.06 (m, 2H), |
| | (trifluoromethyl) | | | | 5.09 (d, J = 10.4 Hz, 1H), |
| | tetrahydrofuran- | | | | 4.83 (t, J = 5.4 Hz, 2H), 4.57 |
| | 2-carboxamide | | | | (d, J = 5.8 Hz, 2H), 4.25 (dd, |
| | | | | | J = 10.4, 7.7 Hz, 1H), 3.96 |
| | | | | | (d, J = 2.3 Hz, 3H), 2.92 (d, J = |
| | | | | | 6.2 Hz, 1H), 2.77 (p, J = |
| | | | | | 7.5 Hz, 1H), 2.06 (d, J = 5.7 |
| | | | | | Hz, 3H), 1.60 (s, 3H), 0.82- |
| | | | | | 0.61 (m, 3H) ppm. |
| 362 | (2R,3S,4S,5R)-3-(3,4- | 529.5 | 530 | 3.24 | ¹H NMR (400 MHz, DMSO- |
| | difluoro-2- | | | | d₆) δ 10.56 (s, 1H), 8.47 (dd, |
| | methoxyphenyl)-N-(2- | | | | J = 5.5, 0.7 Hz, 1H), 7.59- |
| | (3- | | | | 7.50 (m, 2H), 7.23-7.09 (m, |
| | (dimethylamino)oxetan- | | | | 2H), 5.10 (d, J = 10.2 Hz, |
| | 3-yl)pyridin-4-yl)-4,5- | | | | 1H), 4.78 (d, J = 6.2 Hz, 2H), |
| | dimethyl-5- | | | | 4.67 (dd, J = 6.3, 1.3 Hz, |
| | (trifluoromethyl) | | | | 2H), 4.25 (dd, J = 10.3, 7.7 |
| | tetrahydrofuran- | | | | Hz, 1H), 3.95 (d, J = 2.3 Hz, |
| | 2-carboxamide | | | | 3H), 2.77 (p, J = 7.5 Hz, 1H), |
| | | | | | 2.01 (s, 6H), 1.60 (s, 3H), |
| | | | | | 0.73 (dd, J = 7.6, 2.4 Hz, 3H) |
| | | | | | ppm. |
| 363 | (2R,3S,4S,5R)-3-(3,4- | 547.49 | 548 | 3.47 | ¹H NMR (500 MHz, DMSO- |
| | difluoro-2- | | | | d₆) δ 10.34 (s, 1H), 8.57 (d, J = |
| | methoxyphenyl)-N-(2- | | | | 2.2 Hz, 1H), 7.94 (d, J = |
| | (3- | | | | 6.2 Hz, 1H), 7.22-7.15 (m, |
| | (dimethylamino)oxetan- | | | | 2H), 5.34 (d, J = 10.4 Hz, |
| | 3-yl)-5-fluoropyridin-4- | | | | 1H), 4.75 (t, J = 6.2 Hz, 2H), |
| | yl)-4,5-dimethyl-5- | | | | 4.66 (dd, J = 6.2, 2.8 Hz, |
| | (trifluoromethyl) | | | | 2H), 4.23 (dd, J = 10.4, 7.5 |
| | tetrahydrofuran- | | | | Hz, 1H), 3.94 (d, J = 2.0 Hz, |
| | 2-carboxamide | | | | 3H), 2.76 (dq, J = 7.5, 7.5 |
| | | | | | Hz, 1H), 1.99 (s, 6H), 1.60 |
| | | | | | (s, 3H), 0.72 (d, J = 6.5 Hz, |
| | | | | | 3H) ppm. |

The following compounds were made using a method similar to that described in Example 9, except that chiral amines were used in step 1 and an additional SFC purification was run after step 1 to separate diasteriomers. Separated isomers were deprotected using conditions similar to General Method I and then methylated using General Method K as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 364 | rel-(2R*,3S*,4S*,5R*)- | 529.5 | 530.3 | 2.87 | ¹H NMR (500 MHz, DMSO- |
| | 3-(3,4-difluoro-2- | | | | d₆) δ 8.39 (d, J = 5.6 Hz, 1H), |
| | methoxyphenyl)-4,5- | | | | 7.74 (s, 1H), 7.55 (dd, J = |
| | dimethyl-N-(2-(4- | | | | 5.6, 2.1 Hz, 1H), 7.18-7.05 |
| | methylmorpholin-3- | | | | (m, 2H), 5.08 (d, J = 10.2 Hz, |
| | yl)pyridin-4-yl)-5- | | | | 1H), 4.28 (dd, J = 10.2, 8.0 |
| | (trifluoromethyl) | | | | Hz, 1H), 3.96 (d, J = 2.1 Hz, |
| | tetrahydrofuran- | | | | 3H), 3.88 (d, J = 11.5 Hz, |
| | 2-carboxamide | | | | 1H), 3.75 (s, 1H), 3.67 (t, J = |
| | (precursor was first | | | | 11.4 Hz, 1H), 3.35 (s, 2H), |
| | eluting isomer by SFC | | | | 2.95 (s, 1H), 2.78 (t, J = 7.6 |
| | using Lux Cellulose-5 | | | | Hz, 1H), 2.66-2.54 (m, 1H), |
| | column) | | | | 2.15 (s, 3H), 1.62 (s, 3H), |
| | | | | | 0.76 (dq, J = 7.4, 2.3 Hz, 3H) |
| | | | | | ppm. |
| 365 | rel-(2R*,3S*,4S*,5R*)- | 529.5 | 530.4 | 3.35 | ¹H NMR (500 MHz, DMSO- |
| | 3-(3,4-difluoro-2- | | | | d₆) δ 10.55 (s, 1H), 8.35 (d, J = |
| | methoxyphenyl)-4,5- | | | | 5.6 Hz, 1H), 7.75 (d, J = |
| | dimethyl-N-(2-(4- | | | | 2.1 Hz, 1H), 7.51 (dd, J = |
| | methylmorpholin-3- | | | | 5.6, 2.1 Hz, 1H), 7.21-7.09 |
| | yl)pyridin-4-yl)-5- | | | | (m, 2H), 5.08 (d, J = 10.3 Hz, |
| | (trifluoromethyl) | | | | 1H), 4.24 (dd, J = 10.3, 7.7 |
| | tetrahydrofuran- | | | | Hz, 1H), 3.95 (d, J = 2.1 Hz, |
| | 2-carboxamide | | | | 3H), 3.82 (d, J = 8.5 Hz, 1H), |
| | (precursor was second | | | | 3.64 (d, J = 9.6 Hz, 1H), 3.57 |
| | eluting isomer by SFC | | | | (t, J = 11.4 Hz, 1H), 3.19 (t, J = |
| | using Lux Cellulose-5 | | | | 10.5 Hz, 1H), 3.15-3.08 |
| | column) | | | | (m, 1H), 2.84-2.73 (m, 2H), |
| | | | | | 2.32-2.23 (m, 1H), 1.99 (s, |
| | | | | | 3H), 1.60 (s, 3H), 0.75-0.69 |
| | | | | | (m, 3H) ppm. |
| 366 | rel-(2R*,3S*,4S*,5R*)- | 547.49 | 548 | 3.55 | ¹H NMR (500 MHz, DMSO- |
| | 3-(3,4-difluoro-2- | | | | d₆) δ 10.28 (s, 1H), 8.48 (d, J = |
| | methoxyphenyl)-N-(5- | | | | 2.3 Hz, 1H), 8.19 (d, J = |
| | fluoro-2-(4- | | | | 6.4 Hz, 1H), 7.21-7.14 (m, |
| | methylmorpholin-2- | | | | 2H), 5.34 (d, J = 10.4 Hz, |
| | yl)pyridin-4-yl)-4,5- | | | | 1H), 4.49-4.44 (m, 1H), |
| | dimethyl-5- | | | | 4.24 (dd, J = 10.4, 7.5 Hz, |
| | (trifluoromethyl) | | | | 1H), 3.95 (d, J = 2.1 Hz, 3H), |
| | tetrahydrofuran- | | | | 3.96-3.92 (m, 1H), 3.68 (td, |
| | 2-carboxamide | | | | J = 11.4, 2.5 Hz, 1H), 3.05 |
| | (precursor was first | | | | (d, J = 11.4 Hz, 1H), 2.77 |
| | eluting isomer by SFC | | | | (dq, J = 7.5, 7.5 Hz, 1H), |
| | using Chiralpak IC | | | | 2.73-2.63 (m, 1H), 2.22 (s, |
| | column) | | | | 3H), 2.10-2.02 (m, 1H), |
| | | | | | 1.86-1.79 (m, 1H), 1.60 (s, |
| | | | | | 3H), 0.72 (d, J = 6.4 Hz, 3H) |
| | | | | | ppm. |
| 367 | rel-(2R*,3S*,4S*,5R*)- | 547.49 | 548.1 | 3.53 | ¹H NMR (500 MHz, DMSO- |
| | 3-(3,4-difluoro-2- | | | | d₆) δ 10.27 (s, 1H), 8.47 (d, J = |
| | methoxyphenyl)-N-(5- | | | | 2.3 Hz, 1H), 8.18 (d, J = |
| | fluoro-2-(4- | | | | 6.4 Hz, 1H), 7.21-7.14 (m, |
| | methylmorpholin-2- | | | | 2H), 5.34 (d, J = 10.4 Hz, |
| | yl)pyridin-4-yl)-4,5- | | | | 1H), 4.44 (dd, J = 10.3, 2.6 |
| | dimethyl-5- | | | | Hz, 1H), 4.24 (dd, J = 10.4, |
| | (trifluoromethyl) | | | | 7.5 Hz, 1H), 3.95 (d, J = 2.1 |
| | tetrahydrofuran-2- | | | | Hz, 3H), 3.94-3.91 (m, 1H), |
| | carboxamide | | | | 3.66 (td, J = 11.4, 2.6 Hz, |
| | (precursor was second | | | | 1H), 3.04-3.00 (m, 1H), |
| | eluting isomer by SFC | | | | 2.77 (dq, J = 7.5, 7.5 Hz, |
| | using Chiralpak IC | | | | 1H), 2.68-2.64 (m, 1H), |
| | column) | | | | 2.20 (s, 3H), 2.02 (td, J = |
| | | | | | 11.5, 3.4 Hz, 1H), 1.79 (dd, J = |
| | | | | | 11.5, 10.3 Hz, 1H), 1.60 (s, |
| | | | | | 3H), 0.72 (d, J = 6.3 Hz, 3H) |
| | | | | | ppm. |

The following compounds were made using a method similar to that described in Example 9, except that different amines were used in step 1 and General Method J was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 368 | (2R,3S,4S,5R)3-(3,4- | 517.489 | 518.4 | 2.77 | ¹H NMR (500 MHz, DMSO- |
| | difluoro-2- | | | | d₆) δ 10.56-10.47 (m, 1H), |
| | methoxyphenyl)-N-(2- | | | | 8.36 (d, J = 5.6 Hz, 1H), 7.60 |
| | (1-(dimethylamino)-2- | | | | (d, J = 1.9 Hz, 1H), 7.51 (dt, |
| | hydroxyethyl)pyridin-4- | | | | J = 5.6, 2.1 Hz, 1H), 7.21- |
| | yl)-4,5-dimethyl-5- | | | | 7.08 (m, 2H), 5.08 (d, J = |
| | (trifluoromethyl) | | | | 10.2 Hz, 1H), 4.43 (t, J = 5.4 |
| | tetrahydrofuran-2- | | | | Hz, 1H), 4.25 (dd, J = 10.4, |
| | carboxamide, as a | | | | 7.7 Hz, 1H), 3.95 (d, J = 2.0 |
| | mixture of epimers at | | | | Hz, 3H), 3.81-3.74 (m, 1H), |
| | the 2-(1- | | | | 3.74-3.67 (m, 1H), 3.36 (td, |
| | (dimethylamino)-2- | | | | J = 5.8, 2.7 Hz, 1H), 2.81- |
| | hydroxyethyl) group | | | | 2.72 (m, 1H), 2.16 (s, 6H), |
| | | | | | 1.60 (s, 3H), 0.72 (d, J = 7.3 |
| | | | | | Hz, 3H) ppm. |
| 369 | (2R,3S,4S,5R)-3-(3,4- | 518.474 | 519.1 | 3.22 | ¹H NMR (500 MHz, DMSO- |
| | difluoro-2- | | | | d₆) δ 10.48 (s, 1H), 8.33 (d, J = |
| | methoxyphenyl)-N-(2- | | | | 5.6 Hz, 1H), 7.50 (d, J = |
| | ((S)-2-hydroxy-3- | | | | 2.0 Hz, 1H), 7.44 (dd, J = |
| | methoxypropyl)pyridin- | | | | 5.6, 2.0 Hz, 1H), 7.21-7.08 |
| | 4-yl)-4,5-dimethyl-5- | | | | (m, 2H), 5.08 (d, J = 10.3 Hz, |
| | (trifluoromethyl) | | | | 1H), 4.80 (s, 1H), 4.24 (dd, J = |
| | tetrahydrofuran- | | | | 10.3, 7.6 Hz, 1H), 3.95 (d, |
| | 2-carboxamide | | | | J = 2.1 Hz, 4H), 3.25 (d, J = |
| | | | | | 6.9 Hz, 5H), 2.84-2.72 (m, |
| | | | | | 2H), 2.64 (dd, J = 13.6, 8.1 |
| | | | | | Hz, 1H), 1.59 (s, 3H), 0.72 |
| | | | | | (dd, J = 7.7, 2.5 Hz, 3H) |
| | | | | | ppm. |
| 370 | rel-(2R*,3S*,4S*,5R*)- | 535.479 | 536.7 | 3.54 | ¹H NMR (500 MHz, DMSO- |
| | 3-(3,4-difluoro-2- | | | | d₆) δ 10.24 (s, 1H), 8.43 (d, J = |
| | methoxyphenyl)-N-(2- | | | | 2.3 Hz, 1H), 8.18 (d, J = |
| | (2-(dimethylamino)-1- | | | | 6.4 Hz, 1H), 7.22-7.15 (m, |
| | hydroxyethyl)-5- | | | | 2H), 5.33 (d, J = 10.4 Hz, |
| | fluoropyridin-4-yl)-4,5- | | | | 1H), 5.23 (d, J = 4.4 Hz, 1H), |
| | dimethyl-5- | | | | 4.64-4.60 (m, 1H), 4.25 (dd, |
| | (trifluoromethyl) | | | | J = 10.4, 7.5 Hz, 1H), 3.95 |
| | tetrahydrofuran-2- | | | | (d, J = 2.2 Hz, 3H), 2.80- |
| | carboxamide | | | | 2.74 (m, 1H), 2.48 (dd, J = |
| | (2-[1-[tert- | | | | 12.5, 4.4 Hz, 1H), 2.38 (dd, J = |
| | butyl(dimethyl)silyl]oxy | | | | 12.5, 8.1 Hz, 1H), 2.16 (s, |
| | -2- | | | | 6H), 1.60 (s, 3H), 0.72 (d, J = |
| | (dimethylamino)ethyl]- | | | | 6.2 Hz, 3H) ppm. |
| | 5-fluoro-pyridin-4- | | | | |
| | amine used in T3P | | | | |
| | coupling was made from | | | | |
| | tert-butyl N-[5-fluoro-2- | | | | |
| | (oxiran-2-yl)-4- | | | | |
| | pyridyl]carbamate | | | | |
| | (second eluting isomer | | | | |
| | by SFC on Chiralpak IC | | | | |
| | column)) | | | | |
| 371 | rel-(2R*,3S*,4S*,5R*)- | 577.516 | 578.9 | 4.1 | ¹H NMR (500 MHz, DMSO- |
| | 3-(3,4-difluoro-2- | | | | d₆) δ 10.25 (s, 1H), 8.43 (s, |
| | methoxyphenyl)-N-(5- | | | | 1H), 8.20 (d, J = 6.4 Hz, 1H), |
| | fluoro-2-(1-hydroxy-2- | | | | 7.21-7.14 (m, 2H), 5.32 (d, |
| | morpholinoethyl)pyridin- | | | | J = 10.4 Hz, 1H), 5.28 (d, J = |
| | 4-yl)-4,5-dimethyl-5- | | | | 4.5 Hz, 1H), 4.71-4.67 (m, |
| | (trifluoromethyl) | | | | 1H), 4.24 (dd, J = 10.4, 7.5 |
| | tetrahydrofuran-2- | | | | Hz, 1H), 3.95 (d, J = 2.0 Hz, |
| | carboxamide | | | | 3H), 3.52 (t, J = 4.7 Hz, 4H), |
| | (2-[1-[tert- | | | | 2.81-2.73 (m, 1H), 2.56 (dd, |
| | butyl(dimethyl)silyl]oxy- | | | | J = 12.8, 3.8 Hz, 1H), 2.47- |
| | 2-morpholino-ethyl]-5- | | | | 2.39 (m, 4H), 1.60 (s, 3H), |
| | fluoro-pyridin-4-amine | | | | 0.72 (d, J = 6.2 Hz, 3H) ppm. |
| | used in T3P coupling | | | | |
| | was made from tert- | | | | |
| | butyl N-[5-fluoro-2- | | | | |
| | (oxiran-2-yl)-4- | | | | |
| | pyridyl]carbamate | | | | |
| | (second eluting isomer | | | | |
| | by SFC on Chiralpak IC | | | | |
| | column)) | | | | |
| 372 | (2R,3S,4S,5R)-3-(3,4- | 420.334 | 421.1 | 2.97 | ¹H NMR (500 MHz, |
| | difluoro-2- | | | | Methanol-d₄) δ 7.93 (s, 1H), |
| | methoxyphenyl)-4,5- | | | | 7.14 (ddd, J = 8.2, 5.5, 2.1 |
| | dimethyl-N-(1H-1,2,3- | | | | Hz, 1H), 6.97 (td, J = 9.4, 7.6 |
| | triazol-4-yl)-5- | | | | Hz, 1H), 5.11 (d, J = 10.6 |
| | (trifluoromethyl) | | | | Hz, 1H), 4.28 (dd, J = 10.7, |
| | tetrahydrofuran- | | | | 7.9 Hz, 1H), 3.98 (d, J = 2.2 |
| | 2-carboxamide | | | | Hz, 3H), 2.78 (p, J = 7.7 Hz, |
| | | | | | 1H), 1.66 (d, J = 1.3 Hz, 3H), |
| | | | | | 0.81 (dq, J = 7.4, 2.4 Hz, 3H) |
| | | | | | ppm. |

The following compound was made using a method similar to that described in Example 9, except that methyl 5-aminopyridine -2-carboxylate was used as the amine in step 1 and General Method 0 was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 19 | 5-((2R,3S,4S,5R)-3-(3,4- | 474.378 | 475.3 | 2.5 | ¹H NMR (500 MHz, DMSO- |
| | difluoro-2- | | | | d₆) δ 10.69 (s, 1H), 8.82 (d, J = |
| | methoxyphenyl)-4,5- | | | | 2.4 Hz, 1H), 8.15 (dd, J = |
| | dimethyl-5- | | | | 8.6, 2.5 Hz, 1H), 7.95 (d, J = |
| | (trifluoromethyl) | | | | 8.6 Hz, 1H), 7.16 (dd, J = |
| | tetrahydrofuran-2- | | | | 8.6, 4.5 Hz, 2H), 5.14 (d, J = |
| | carboxamido)picolinic | | | | 10.3 Hz, 1H), 4.26 (dd, J = |
| | acid | | | | 10.3, 7.6 Hz, 1H), 3.95 (d, J = |
| | | | | | 2.0 Hz, 3H), 2.77 (p, J = |
| | | | | | 7.6 Hz, 1H), 1.61 (s, 3H), |
| | | | | | 0.73 (d, J = 4.7 Hz, 3H) ppm. |

The following compound was made from 19 using conditions similar to General Method L. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 1 | 5-((2R,3S,4S,5R)-3-(3,4- | 473.393 | 474.15 | 2.95 | ¹H NMR (500 MHz, |
| | difluoro-2- | | | | Chloroform-d) δ 8.69 (t, J = |
| | methoxyphenyl)-4,5- | | | | 1.7 Hz, 1H), 8.52 (s, 1H), 8.18 |
| | dimethyl-5- | | | | (d, J = 2.0 Hz, 2H), 7.70 (s, |
| | (trifluoromethyl) | | | | 1H), 7.09 (ddd, J = 8.1, 5.5, |
| | tetrahydrofuran-2- | | | | 2.0 Hz, 1H), 6.91 (td, J = 9.2, |
| | carboxamido)picolinamide | | | | 7.4 Hz, 1H), 5.51 (s, 1H), 5.05 |
| | | | | | (d, J = 10.9 Hz, 1H), 4.11 (dd, |
| | | | | | J = 11.0, 8.1 Hz, 1H), 4.01 (d, |
| | | | | | J = 2.7 Hz, 3H), 2.76 (p, J = |
| | | | | | 7.7 Hz, 1H), 1.69 (s, 3H), 0.80 |
| | | | | | (dq, J = 7.5, 2.4 Hz, 3H) ppm. |

The following compound was made from 1 using General Method A. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 2 | 2-carbamoyl-5- | 489.393 | 490.2 | 3.17 | ¹H NMR (500 MHz, DMSO- |
| | ((2R,3S,4S,5R)-3-(3,4- | | | | d₆) δ 10.70 (s, 1H), 10.11 (d, J = |
| | difluoro-2- | | | | 4.5 Hz, 1H), 8.86 (d, J = 2.0 |
| | methoxyphenyl)-4,5- | | | | Hz, 1H), 8.16 (d, J = 9.0 Hz, |
| | dimethyl-5- | | | | 1H), 8.13 (d, J = 4.3 Hz, 1H), |
| | (trifluoromethyl) | | | | 7.73 (dd, J = 9.0, 2.0 Hz, 1H), |
| | tetrahydrofuran-2- | | | | 7.21-7.12 (m, 2H), 5.12 (d, J = |
| | carboxamido)pyridinel- | | | | 10.1 Hz, 1H), 4.25 (dd, J = |
| | oxide | | | | 10.1, 7.7 Hz, 1H), 3.94 (d, J = |
| | | | | | 2.0 Hz, 3H), 2.77 (p, J = 7.5 |
| | | | | | Hz, 1H), 1.60 (s, 3H), 0.78- |
| | | | | | 0.66 (m, 3H) ppm. |

The following compound was made from 19 via an amide coupling using cyanamide, HATU and DIPEA in DMF (similar conditions to those described in Example 8). In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 373 | N-cyano-5- | 498.403 | 499.4 | 2.63 | ¹H NMR (500 MHz, DMSO- |
| | ((2R,3S,4S,5R)-3-(3,4- | | | | d₆) δ 10.57 (s, 1H), 8.79 (s, |
| | difluoro-2- | | | | 1H), 8.11 (d, J = 8.8 Hz, 1H), |
| | methoxyphenyl)-4,5- | | | | 7.97 (d, J = 8.6 Hz, 1H), 7.23- |
| | dimethyl-5- | | | | 6.93 (m, 4H), 5.12 (d, J = |
| | (trifluoromethyl) | | | | 10.2 Hz, 1H), 4.26 (dd, J = |
| | tetrahydrofuran-2- | | | | 10.2, 7.7 Hz, 1H), 3.95 (d, J = |
| | carboxamido)picolinamide | | | | 2.0 Hz, 3H), 2.77 (p, J = |
| | (ammonia salt) | | | | 7.5 Hz, 1H), 1.61 (s, 3H), |
| | | | | | 0.73 (d, J = 7.2 Hz, 3H). |

The following compound was made from 19 via an amide coupling using N-benzyloxymethanamine, HATU and DIPEA in DMF (similar conditions to those described in Example 8). Benzyl deprotection using General Method R with wet Deguassa Pd/C was carried out as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 374 | 5-((2R,3S,4S,5R)-3-(3,4- | 503.419 | 504.2 | 3.38 | ¹H NMR (500 MHz, DMSO- |
| | difluoro-2- | | | | d₆) δ 10.55 (s, 1H), 10.26 (s, |
| | methoxyphenyl)-4,5- | | | | 1H), 8.79 (d, J = 2.5 Hz, 1H), |
| | dimethyl-5- | | | | 8.13 (d, J = 8.6 Hz, 1H), 7.58 |
| | (trifluoromethyl) | | | | (s, 1H), 7.24-7.10 (m, 2H), |
| | tetrahydrofuran- | | | | 5.12 (d, J = 10.2 Hz, 1H), |
| | 2-carboxamido)- | | | | 4.26 (dd, J = 10.2, 7.7 Hz, |
| | N-hydroxy-N- | | | | 1H), 3.95 (d, J = 2.1 Hz, 3H), |
| | methylpicolinamide | | | | 3.28 (s, 3H), 2.77 (p, J = 7.6 |
| | | | | | Hz, 1H), 1.61 (s, 3H), 0.73 |
| | | | | | (d, J = 7.4 Hz, 3H) ppm. |

The following compounds were made from 19 via a coupling reaction using methansulfonamide or N-methylmethanesulfonamide (respectively) with EDC and DMAP in DCM (similar conditions to those described in Example 13). In the Table below, “MS r.t.” stands for Mass Spec

| Cmpd | | LC/MS | Found | MS | |
|---|---|---|---|---|---|
| No. | Compound Name | (m/z calc.) | M + 1 | r.t. | NMR (shifts in ppm) |
| 375 | 5-((2R,3S,4S,5R)-3-(3,4- | 551.484 | 552.3 | 3.64 | ¹H NMR (500 MHz, DMSO- |
| | difluoro-2- | | | | d₆) δ 10.76 (s, 1H), 8.92 (d, J = |
| | methoxyphenyl)-4,5- | | | | 2.4 Hz, 1H), 8.30 (dd, J = |
| | dimethyl-5- | | | | 8.6, 2.4 Hz, 1H), 8.07 (d, J = |
| | (trifluoromethyl) | | | | 8.6 Hz, 1H), 7.22-7.12 (m, |
| | tetrahydrofuran-2- | | | | 2H), 5.14 (d, J = 10.2 Hz, |
| | carboxamido)-N- | | | | 1H), 4.27 (dd, J = 10.2, 7.7 |
| | (methylsulfonyl)picolinamide | | | | Hz, 1H), 3.95 (d, J = 2.0 Hz, |
| | | | | | 3H), 3.35 (s, 3H), 2.78 (p, J = |
| | | | | | 7.5 Hz, 1H), 1.61 (s, 3H), |
| | | | | | 0.74 (dd, J = 7.5, 2.6 Hz, 3H) |
| | | | | | ppm. |
| 376 | 5-((2R,3S,4S,5R)-3-(3,4- | 565.51 | | 3.55 | ¹H NMR (500 MHz, DMSO- |
| | difluoro-2- | | | | d₆) δ 10.66 (s, 1H), 8.86 (d, J = |
| | methoxyphenyl)-4,5- | | | | 2.4 Hz, 1H), 8.20 (dd, J = |
| | dimethyl-5- | | | | 8.6, 2.5 Hz, 1H), 7.74 (d, J = |
| | (trifluoromethyl) | | | | 8.6 Hz, 1H), 7.17 (dd, J = |
| | tetrahydrofuran-2- | | | | 8.7, 4.1 Hz, 2H), 5.14 (d, J = |
| | carboxamido)- | | | | 10.2 Hz, 1H), 4.27 (dd, J = |
| | N-methyl-N- | | | | 10.2, 7.7 Hz, 1H), 3.95 (d, J = |
| | (methylsulfonyl)picolinamide | | | | 2.0 Hz, 3H), 3.56 (s, 3H), |
| | | | | | 3.24 (s, 3H), 2.78 (p, J = 7.5 |
| | | | | | Hz, 1H), 1.61 (s, 3H), 0.74 |
| | | | | | (d, J = 7.3 Hz, 3H) ppm. |

The following compound was made using conditions similar to that described in Example 9, except 5-fluoro-2-vinylpyridin-4-amine was used as the amine coupling partner. The product was reacted using conditions similar to General Method T, taking forward the first eluting isomer from the SFC (step 2) and treating with TBAF in step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 24 | rel-(2R*,3S*,4S*,5R*)-3- | 510.402 | 510.9 | 3.42 | ¹H NMR (500 MHz, DMSO- |
| | (3,4-difluoro-2- | | | | d₆) δ 10.27 (s, 1H), 8.49 (d, J = |
| | methoxyphenyl)-N-(5- | | | | 2.3 Hz, 1H), 8.27 (d, J = |
| | fluoro-2-(2-fluoro-1- | | | | 6.4 Hz, 1H), 7.22-7.15 (m, |
| | hydroxyethyl)pyridin-4- | | | | 2H), 5.95 (d, J = 5.0 Hz, 1H), |
| | yl)-4,5-dimethyl-5- | | | | 5.34 (d, J = 10.4 Hz, 1H), |
| | (trifluoromethyl) | | | | 4.82-4.74 (m, 1H), 4.62 |
| | tetrahydrofuran- | | | | (ddd, J = 47.7, 9.5, 3.1 Hz, |
| | 2-carboxamide | | | | 1H), 4.48 (ddd, J = 47.7, 9.5, |
| | | | | | 6.0 Hz, 1H), 4.25 (dd, J = |
| | | | | | 10.4, 7.6 Hz, 1H), 3.95 (s, |
| | | | | | 3H), 2.81-2.73 (m, 1H), |
| | | | | | 1.61 (s, 3H), 0.73 (d, J = 5.2 |
| | | | | | Hz, 3H) ppm. |

The following compound was made using conditions similar to that described in Example 9, except 5-fluoro-2-vinylpyridin-4-amine was used as the amine coupling partner. The product was reacted using conditions similar to General Method T except, the first eluting isomer by SFC from step 2 was ring opened by treated with HNMe₂ in water, ethanol and THF at ambient temperature in step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 377 | rel-(2R*,3S*,4S*,5R*)-3- | 535.479 | 536.7 | 3.54 | ¹H NMR (500 MHz, DMSO- |
| | (3,4-difluoro-2- | | | | d₆) δ 10.24 (s, 1H), 8.43 (d, J = |
| | methoxyphenyl)-N-(2-(2- | | | | 2.5 Hz, 1H), 8.18 (d, J = |
| | (dimethylamino)-1- | | | | 6.4 Hz, 1H), 7.22-7.15 (m, |
| | hydroxyethyl)-5- | | | | 2H), 5.33 (d, J = 10.5 Hz, |
| | fluoropyridin-4-yl)-4,5- | | | | 1H), 5.22 (d, J = 4.4 Hz, 1H), |
| | dimethyl-5- | | | | 4.65-4.57 (m, 1H), 4.25 (dd, |
| | (trifluoromethyl) | | | | J = 10.5, 7.5 Hz, 1H), 3.95 |
| | tetrahydrofuran- | | | | (d, J = 2.0 Hz, 3H), 2.81- |
| | 2-carboxamide | | | | 2.73 (m, 1H), 2.48 (dd, J = |
| | | | | | 12.5, 4.4 Hz, 1H), 2.39 (dd, J = |
| | | | | | 12.5, 8.1 Hz, 1H), 2.17 (s, |
| | | | | | 6H), 1.61 (s, 3H), 0.72 (d, J = |
| | | | | | 6.1 Hz, 3H) ppm. |

The following compound was made using conditions similar to that described in Example 9, except 2-vinylpyridin-4-amine was used as the amine coupling partner. The product was reacted using conditions similar to General Method T except the step 2 SFC was omitted and the mixture of isomers generated in step 1 was treated with methylamine in water in step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 378 | (2R,3S,4S,5R)-3-(3,4- | 517.489 | 518.3 | 2.62 | ¹H NMR (500 MHz, DMSO- |
| | difluoro-2- | | | | d₆) δ 10.55 (s, 1H), 8.34 (d, J = |
| | methoxyphenyl)-N-(2-(2- | | | | 5.6 Hz, 1H), 7.74 (d, J = |
| | (dimethylamino)-1- | | | | 2.0 Hz, 1H), 7.49 (dd, J = |
| | hydroxyethyl)pyridin-4- | | | | 5.5, 2.2 Hz, 1H), 7.24-7.08 |
| | yl)-4,5-dimethyl-5- | | | | (m, 2H), 5.17 (d, J = 4.0 Hz, |
| | (trifluoromethyl) | | | | 1H), 5.09 (d, J = 10.3 Hz, |
| | tetrahydrofuran- | | | | 1H), 4.63 (dt, J = 8.2, 3.9 Hz, |
| | 2-carboxamide, as | | | | 1H), 4.25 (dd, J = 10.3, 7.6 |
| | a mixture of epimers | | | | Hz, 1H), 3.97-3.94 (m, 3H), |
| | | | | | 2.78 (p, J = 7.5 Hz, 1H), 2.40 |
| | | | | | (dd, J = 12.5, 8.3 Hz, 1H), |
| | | | | | 2.20 (d, J = 0.9 Hz, 6H), 2.05- |
| | | | | | 1.97 (m, 1H), 1.61 (s, 3H), |
| | | | | | 0.76-0.70 (m, 3H) ppm. |

### Example 10

(2R,3S,4S,5R)-3-(3,4-difluoro-2-methoxyphenyl)-4,5-dimethyl-N-(2-(4-methyl-2-oxopiperazin-1-yl)pyridin-4-yl)-5-(trifluoromethyl)tetrahydrofuran-2-carboxamide (379)

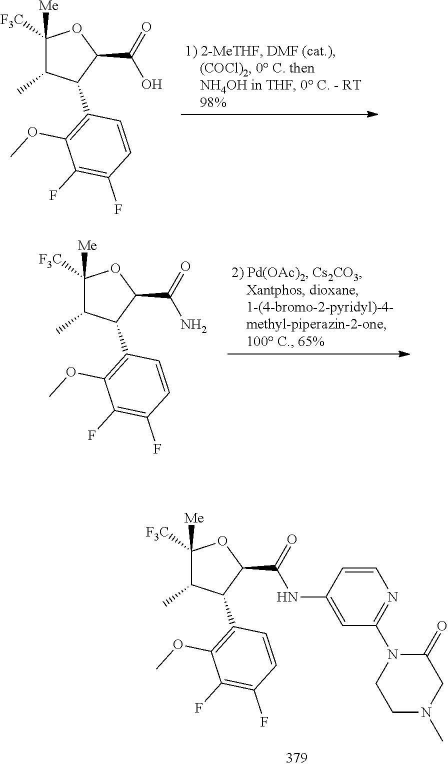 [SMILES: COc1c([C@H]2[C@H](C(=O)O)O[C@@](C)(C(F)(F)F)[C@H]2C)ccc(F)c1F ; COc1c([C@H]2[C@H](C(N)=O)O[C@@](C)(C(F)(F)F)[C@H]2C)ccc(F)c1F ; COc1c([C@H]2[C@H](C(=O)Nc3ccnc(N4CCN(C)CC4=O)c3)O[C@@](C)(C(F)(F)F)[C@H]2C)ccc(F)c1F]

### Step 1:

To an ice cooled solution of (2R,3S,4S,5R)-3-(3,4-difluoro-2-methoxyphenyl)-4,5-dimethyl-5-(trifluoromethyl)tetrahydrofuran-2-carboxylic acid (2390 mg, 6.409 mmol) in 2-methyltetrahydrofuran (20 mL) was added DMF (60 μL, 0.7749 mmol) and carefully oxalyl chloride (1.1 mL, 12.61 mmol) and warmed to ambient temperature and stirred for 90 minutes. The reaction mixture was concentrated in vacuo and the residue dissolved in 2-methyltetrahydrofuran (10 mL). This solution was added to an ice cooled solution of ammonium hydroxide (10 mL of 28% w/v, 79.90 mmol) in 2-methyltetrahydrofuran (10 mL). The resulting mixture was stirred and warmed to ambient temperature over 1.5 hours. The reaction mixture was quenched with water (15 mL) and partitioned with water and EtOAc. The layers were separated and the organic washed with brine before passing through a phase separation cartridge and concentrating in-vacuo to afford (2R,3S,4S,5R)-3-(3,4-difluoro-2-methoxyphenyl)-4,5-dimethyl-5-(trifluoromethyl)tetrahydrofuran-2-carboxamide (2.34 g, 98%) as yellow oil. ¹H NMR (400 MHz, DMSO-d₆) δ 7.51 (s, 1H), 7.33 (s, 1H), 7.23-7.04 (m, 2H), 4.83 (d, J=10.7 Hz, 1H), 4.11-3.97 (m, 1H), 3.94 (d, J=2.2 Hz, 3H), 2.66 (p, J=7.5 Hz, 1H), 1.56 (d, J=1.2 Hz, 3H), 0.75-0.63 (m, 3H) ppm. ESI-MS m/z calc. 353.10504, found 354.0 (M+1)⁺; Retention time: 0.87 minutes.

### Step 2:

To a solution of (2R,3S,4S,5R)-3-(3,4-difluoro-2-methoxyphenyl)-4,5-dimethyl-5-(trifluoromethyl)tetrahydrofuran-2-carboxamide (360 mg, 0.9681 mmol) in dioxane (5.5 mL) was added 1-(4-bromo-2-pyridyl)-4-methyl-piperazin-2-one (321 mg, 1.188 mmol), Xantphos (107 mg, 0.1849 mmol) and cesium carbonate (633 mg, 1.943 mmol). Vial was degassed and purged with N₂ before addition of Pd(OAc)₂ (25 mg, 0.1114 mmol). The vial was sealed and heated at 100° C. for 20 hours and then left at ambient temperature for 3 days. The mixture was filtered through a celite cartridge (2 g), washing with EtOAc and concentrated in-vacuo. It was then taken up in MeOH and loaded on to a 10 g SCX-2 cartridge, washed with MeOH (6 mL) before eluting the desired product with 2M NH₃ in MeOH (6 mL). The basic eluent was concentrated in-vacuo to afford a light brown gum. The residue was purified by flash column chromatography (24 g SiO₂, 50 to 100% EtOAc (with 1% NH₃OH) in hexanes) afford (2R,3S,4S,5R)-3-(3,4-difluoro-2-methoxyphenyl)-4,5-dimethyl-N-(2-(4-methyl-2-oxopiperazin-1-yl)pyridin-4-yl)-5-(trifluoromethyl)tetrahydrofuran-2-carboxamide (379, 360 mg, 65%) as a white solid after freeze-drying. ¹H NMR (500 MHz, DMSO-d₆) δ 10.66 (s, 1H), 8.30 (d, J=5.6 Hz, 1H), 8.09 (d, 1H), 7.52 (dd, J=5.6, 1.9 Hz, 1H), 7.22-7.07 (m, 2H), 5.09 (d, J=10.2 Hz, 1H), 4.24 (dd, J=10.3, 7.6 Hz, 1H), 3.95 (d, J=2.0 Hz, 3H), 3.88-3.76 (m, 2H), 3.16 (s, 2H), 2.81-2.73 (m, 1H), 2.70 (t, J=5.6 Hz, 2H), 2.27 (s, 3H), 1.59 (s, 3H), 0.77-0.67 (m, 3H) ppm. ESI-MS m/z calc. 542.19525, found 543.2 (M+1)⁺; 541.3 (M−1)⁻; Retention time: 3.17 minutes.

The following compounds were made using a method similar to that described in Example 10, except that different coupling partners were used in step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 380 | (2R,3S,4S,5R)-3-(3,4- | 592.579 | 593 | 3.4 | ¹H NMR (500 MHz, DMSO-d₆) |
| | difluoro-2- | | | | δ 10.69 (s, 1H), 9.05 (d, J = 2.3 |
| | methoxyphenyl)-4,5- | | | | Hz, 1H), 8.60 (d, J = 2.0 Hz, |
| | dimethyl-N-(5-((4- | | | | 1H), 8.47 (t, J = 2.2 Hz, 1H), |
| | methylpiperazin-1- | | | | 7.22-7.11 (m, 2H), 5.14 (d, J = |
| | yl)sulfonyl)pyridin-3- | | | | 10.2 Hz, 1H), 4.26 (dd, J = 10.2, |
| | yl)-5- | | | | 7.7 Hz, 1H), 3.95 (d, J = 2.2 Hz, |
| | (trifluoromethyl) | | | | 3H), 2.95 (t, J = 4.9 Hz, 4H), |
| | tetrahydrofuran-2- | | | | 2.78 (p, J = 7.5 Hz, 1H), 2.35 (t, |
| | carboxamide | | | | J = 4.9 Hz, 4H), 2.13 (s, 3H), |
| | | | | | 1.62 (s, 3H), 0.79-0.65 (m, 3H) |
| | | | | | ppm. |
| 381 | (2R,3S,4S,5R)-3-(3,4- | 461.383 | 462.3 | 3.18 | ¹H NMR (500 MHz, DMSO-d₆) |
| | difluoro-2- | | | | δ 11.11 (s, 1H), 8.66 (d, J = 5.7 |
| | methoxyphenyl)-N-(2- | | | | Hz, 1H), 7.90 (d, J = 5.7 Hz, |
| | (hydroxymethyl) | | | | 1H), 7.20 (dd, J = 8.5, 4.4 Hz, |
| | pyrimidin-4-yl)-4,5- | | | | 2H), 5.27 (d, J = 10.4 Hz, 1H), |
| | dimethyl-5- | | | | 5.23 (t, J = 6.2 Hz, 1H), 4.54 (d, |
| | (trifluoromethyl) | | | | J = 6.1 Hz, 2H), 4.25 (dd, J = |
| | tetrahydrofuran-2- | | | | 10.5, 7.6 Hz, 1H), 3.95 (d, J = |
| | carboxamide | | | | 2.2 Hz, 3H), 2.77 (t, J = 7.5 Hz, |
| | | | | | 1H), 1.60 (s, 3H), 0.75-0.68 |
| | | | | | (m, 3H) ppm. |
| 382 | (2R,3S,4S,5R)-3-(3,4- | 461.383 | 462.4 | 3.18 | ¹H NMR (500 MHz, DMSO-d₆) |
| | difluoro-2- | | | | δ 10.99 (s, 1H), 9.17 (s, 1H), |
| | methoxyphenyl)-N-(6- | | | | 8.46 (s, 1H), 7.25-7.15 (m, |
| | (hydroxymethyl)pyrazin- | | | | 2H), 5.58 (t, J = 5.8 Hz, 1H), |
| | 2-yl)-4,5-dimethyl- | | | | 5.25 (d, J = 10.5 Hz, 1H), 4.58 |
| | 5- | | | | (d, J = 5.7 Hz, 2H), 4.27 (dd, J = |
| | (trifluoromethyl) | | | | 10.5, 7.6 Hz, 1H), 3.95 (d, J = |
| | tetrahydrofuran-2- | | | | 2.1 Hz, 3H), 2.77 (t, J = 7.5 Hz, |
| | carboxamide | | | | 1H), 1.61 (s, 3H), 0.79-0.66 |
| | | | | | (m, 3H) ppm. |
| 383 | (2R,3S,4S,5R)-3-(3,4- | 460.394 | 461.3 | 3.13 | ¹H NMR (500 MHz, Methanol- |
| | difluoro-2- | | | | d₄) δ 8.66 (d, J = 2.4 Hz, 1H), |
| | methoxyphenyl)-N-(5- | | | | 8.27 (d, J = 1.8 Hz, 1H), 8.11 (t, |
| | (hydroxymethyl)pyridin- | | | | J = 2.2 Hz, 1H), 7.13 (ddd, J = |
| | 3-yl)-4,5-dimethyl-5- | | | | 8.1, 5.5, 2.1 Hz, 1H), 7.04-6.90 |
| | (trifluoromethyl) | | | | (m, 1H), 5.08 (d, J = 10.5 Hz, |
| | tetrahydrofuran-2- | | | | 1H), 4.63 (s, 2H), 4.31 (dd, J = |
| | carboxamide | | | | 10.5, 8.0 Hz, 1H), 4.00 (d, J = |
| | | | | | 2.2 Hz, 3H), 2.79 (p, J = 7.7 Hz, |
| | | | | | 1H), 1.67 (d, J = 1.3 Hz, 3H), |
| | | | | | 0.82 (dt, J = 7.4, 2.4 Hz, 3H) |
| | | | | | ppm. |
| 384 | (2R,3S,4S,5R)-3-(3,4- | 557.513 | 558.14 | 2.27 | ¹H NMR (400 MHz, |
| | difluoro-2- | | | | Chloroform-d) d 9.29 (s, 1H), |
| | methoxyphenyl)-N-(6- | | | | 8.48 (s, 1H), 8.09 (s, 1H), 7.01- |
| | ((R)-2,4-dimethyl-6- | | | | 6.94 (m, 1H), 6.87-6.78 (m, |
| | oxopiperazin-1- | | | | 1H), 5.01-4.95 (m, 2H), 4.05- |
| | yl)pyridazin-4-yl)-4,5- | | | | 3.95 (m, 4H), 3.63-3.46 (m, |
| | dimethyl-5- | | | | 1H), 3.18-3.03 (m, 1H), 2.91- |
| | (trifluoromethyl) | | | | 2.66 (m, 3H), 2.41 (s, 3H), 1.60 |
| | tetrahydrofuran-2- | | | | (s, 3H), 1.22 (d, J = 10.8 Hz, 3H), |
| | carboxamide | | | | 0.73 (d, J = 6.2 Hz, 3H) ppm. |

The following compounds were made using a method similar to that described in Example 10, except that different coupling partners were used in step 2 and 2-methyl tetrahydrofuran was used as solvent in place of dioxane. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 385 | (2R,3S,4S,5R)-3-(3,4- | 474.421 | 475.6 | 3.15 | ¹H NMR (500 MHz, DMSO-d₆) |
| | difluoro-2- | | | | δ 10.48 (s, 1H), 8.34 (d, J = 5.5 |
| | methoxyphenyl)-N-(2- | | | | Hz, 1H), 7.50 (d, J = 2.0 Hz, |
| | (2- | | | | 1H), 7.44 (dd, J = 5.6, 2.1 Hz, |
| | hydroxyethyl)pyridin- | | | | 1H), 7.24-7.02 (m, 2H), 5.09 |
| | 4-yl)-4,5-dimethyl-5- | | | | (d, J = 10.3 Hz, 1H), 4.61 (t, J = |
| | (trifluoromethyl) | | | | 5.3 Hz, 1H), 4.25 (dd, J = 10.3, |
| | tetrahydrofuran-2- | | | | 7.6 Hz, 1H), 3.96 (d, J = 2.1 Hz, |
| | carboxamide | | | | 3H), 3.71 (td, J = 6.7, 5.2 Hz, |
| | | | | | 2H), 2.88-2.71 (m, 2H), 2.05- |
| | | | | | 1.73 (m, 1H), 1.60 (s, 3H), 0.80- |
| | | | | | 0.58 (m, 3H) ppm. |
| 386 | (2R,3S,4S,5R)-3-(3,4- | 543.526 | | 3.33 | ¹H NMR (500 MHz, DMSO-d₆) |
| | difluoro-2- | | | | δ 10.54 (s, 1H), 8.31 (d, J = 5.6 |
| | methoxyphenyl)-N-(2- | | | | Hz, 1H), 7.61 (d, J = 2.1 Hz, |
| | (((S)-2- | | | | 1H), 7.52 (dd, J = 5.6, 2.1 Hz, |
| | (hydroxymethyl) | | | | 1H), 7.21-7.14 (m, 1H), 7.14- |
| | pyrrolidin-1- | | | | 7.08 (m, 1H), 5.07 (d, J = 10.2 |
| | yl)methyl)pyridin-4- | | | | Hz, 1H), 4.44 (t, J = 5.3 Hz, |
| | yl)-4,5-dimethyl-5- | | | | 1H), 4.25 (dd, J = 10.3, 7.7 Hz, |
| | (trifluoromethyl) | | | | 1H), 4.07 (d, J = 14.4 Hz, 1H), |
| | tetrahydrofuran-2- | | | | 3.95 (d, J = 2.0 Hz, 3H), 3.49- |
| | carboxamide | | | | 3.39 (m, 2H), 3.29-3.23 (m, |
| | | | | | 1H), 2.86 (ddd, J = 9.5, 6.8, 3.1 |
| | | | | | Hz, 1H), 2.77 (p, J = 7.5 Hz, |
| | | | | | 1H), 2.66-2.58 (m, 1H), 2.21 |
| | | | | | (td, J = 9.0, 7.3 Hz, 1H), 1.89- |
| | | | | | 1.79 (m, 1H), 1.68-1.48 (m, |
| | | | | | 6H), 0.72 (dd, J = 7.3, 2.4 Hz, |
| | | | | | 3H) ppm. |
| 387 | (2R,3S,4S,5R)-3-(3,4- | 459.41 | 460 | 3.35 | ¹H NMR (400 MHz, DMSO-d₆) |
| | difluoro-2- | | | | δ 10.21 (s, 1H), 7.84 (d, J = 5.6 |
| | methoxyphenyl)-4,5- | | | | Hz, 1H), 7.23-7.13 (m, 1H), |
| | dimethyl-N-(2- | | | | 7.10 (t, J = 7.3 Hz, 1H), 6.83 (d, |
| | (methylamino)pyridin- | | | | J = 1.9 Hz, 1H), 6.62 (dd, J = |
| | 4-yl)-5- | | | | 5.6, 1.8 Hz, 1H), 6.40 (t, J = 4.9 |
| | (trifluoromethyl) | | | | Hz, 1H), 5.04 (d, J = 10.3 Hz, |
| | tetrahydrofuran-2- | | | | 1H), 4.23 (dd, J = 10.3, 7.6 Hz, |
| | carboxamide | | | | 1H), 3.95 (d, J = 2.2 Hz, 3H), |
| | | | | | 2.77 (q, J = 7.6 Hz, 1H), 2.71 (d, |
| | | | | | J = 4.9 Hz, 3H), 1.58 (d, J = 5.3 |
| | | | | | Hz, 3H), 0.76-0.69 (m, 3H) |
| | | | | | ppm. |
| 388 | (2R,3S,4S,5R)-N- | 471.381 | 472.3 | 3.4 | ¹H NMR (500 MHz, DMSO-d₆) |
| | ([1,2,4]triazolo[4,3- | | | | δ 10.71 (s, 1H), 9.66 (d, J = 2.5 |
| | a]pyrimidin-6-yl)-3- | | | | Hz, 1H), 8.95 (d, J = 2.6 Hz, |
| | (3,4-difluoro-2- | | | | 1H), 8.61 (s, 1H), 7.24-7.09 |
| | methoxyphenyl)-4,5- | | | | (m, 2H), 5.18 (d, J = 10.3 Hz, |
| | dimethyl-5- | | | | 1H), 4.27 (dd, J = 10.3, 7.7 Hz, |
| | (trifluoromethyl) | | | | 1H), 3.95 (d, J = 1.9 Hz, 3H), |
| | tetrahydrofuran-2- | | | | 2.83-2.74 (m, 1H), 1.64 (s, |
| | carboxamide | | | | 3H), 0.78-0.69 (m, 3H) ppm. |
| 389 | (2R,3S,4S,5R)-3-(3,4- | 489.436 | 490.235 | 2.67 | 1H-NMR (400 MHz, |
| | difluoro-2-methoxy- | | | | CHLOROFORM-D) δ 9.03 (brs, |
| | phenyl)-N-[6-(1- | | | | 1H), 8.83-8.82 (m, 1H), 8.16 (d, |
| | hydroxy-1-methyl- | | | | J = 0.9 Hz, 1H), 7.08-7.04 (m, |
| | ethyl)pyrimidin-4-yl]- | | | | 1H), 6.90 (td, J = 9.3, 7.6 Hz, |
| | 4,5-dimethyl-5- | | | | 1H), 5.02 (d, J = 11.0 Hz, 1H), |
| | (trifluoromethyl) | | | | 4.21 (s, 1H), 4.10 (dd, J = 11.0, |
| | tetrahydrofuran-2- | | | | 8.2 Hz, 1H), 4.00 (d, J = 2.7 Hz, |
| | carboxamide | | | | 3H), 2.78-2.70 (m, 1H), 1.68 (s, |
| | | | | | 3H), 1.49 (dd, J = 14.9, 6.6 Hz, |
| | | | | | 6H), 0.78-0.76 (m, 3H) |
| 390 | (2R,3S,4S,5R)-3-(3,4- | 489.436 | 490.22 | 2.59 | 1H-NMR (400 MHz, |
| | difluoro-2- | | | | Chloroform-d) δ 8.95 (s, 2H), |
| | methoxyphenyl)-N-(2- | | | | 8.35 (s, 1H), 7.09-7.05 (m, 1H), |
| | (2-hydroxypropan-2- | | | | 6.94-6.87 (m, 1H), 5.04 (d, J = |
| | yl)pyrimidin-5-yl)-4,5- | | | | 11.0 Hz, 1H), 4.10 (dd, J = 11.0, |
| | dimethyl-5- | | | | 7.8 Hz, 1H), 4.00 (d, J = 3.2 Hz, |
| | (trifluoromethyl) | | | | 3H), 2.76 (t, J = 7.8 Hz, 1H), |
| | tetrahydrofuran-2- | | | | 1.68 (s, 3H), 1.57 (s, 6H), 0.80- |
| | carboxamide | | | | 0.78 (m, 3H) ppm. One |
| | | | | | hydroxyl proton not located. |

The following compounds were made using a method similar to that described in Example 10, except that different coupling partners were used in step 2 and Pd₂(dba)₃ was used as catalyst. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 391 | (2R,3S,4S,5R)-3-(3,4- | 542.498 | 543 | 3.19 | ¹H NMR (500 MHz, DMSO- |
| | difluoro-2- | | | | d₆) δ 10.45 (s, 1H), 8.65 (d, J = |
| | methoxyphenyl)-4,5- | | | | 2.6 Hz, 1H), 8.02 (dd, J = 9.0, |
| | dimethyl-N-(6-(4- | | | | 2.7 Hz, 1H), 7.90-7.67 (m, |
| | methyl-2- | | | | 1H), 7.23-7.03 (m, 2H), 5.10 |
| | oxopiperazin-1- | | | | (d, J = 10.3 Hz, 1H), 4.25 (dd, |
| | yl)pyridin-3-yl)-5- | | | | J = 10.3, 7.7 Hz, 1H), 3.95 (d, |
| | (trifluoromethyl)- | | | | J = 2.0 Hz, 3H), 3.85-3.78 |
| | tetrahydrofuran-2- | | | | (m, 2H), 3.16 (s, 2H), 2.78 (q, |
| | carboxamide | | | | J = 7.5 Hz, 1H), 2.73-2.67 |
| | | | | | (m, 2H), 2.28 (s, 3H), 1.61 (s, |
| | | | | | 3H), 0.81-0.69 (m, 3H) ppm. |
| 392 | (2R,3S,4S,5R)-3-(3,4- | 514.485 | 515.3 | 3.40 | 1H NMR (400 MHZ, |
| | difluoro-2- | | | | Chloroform-d) δ 8.55-8.49 |
| | methoxyphenyl)-4,5- | | | | (m, 1H), 8.36 (s, 1H), 8.07 |
| | dimethyl-N-(6- | | | | (ddd, J = 8.4, 2.7, 1.8 Hz, 1H), |
| | ((tetrahydrofuran-3- | | | | 7.11 (dd, J = 8.4, 4.9 Hz, 2H), |
| | yl)methyl)pyridin-3- | | | | 6.92 (td, J = 9.2, 7.4 Hz, 1H), |
| | yl)-5- | | | | 5.04 (d, J = 10.9 Hz, 1H), 4.12 |
| | (trifluoromethyl)- | | | | (dd, J = 10.9, 8.0 Hz, 1H), 4.02 |
| | tetrahydrofuran-2- | | | | (d, J = 2.7 Hz, 3H), 3.96-3.82 |
| | carboxamide, as a | | | | (m, 2H), 3.78 (dt, J = 8.5, 7.4 |
| | mixture of epimers | | | | Hz, 1H), 3.49 (ddd, J = 8.5, |
| | | | | | 6.5, 1.1 Hz, 1H), 2.90-2.79 |
| | | | | | (m, 2H), 2.81-2.62 (m, 2H), |
| | | | | | 2.08-1.95 (m, 1H), 1.69 (d, J = |
| | | | | | 9.8 Hz, 3H), 1.64 (dtd, J = |
| | | | | | 12.3, 7.6, 7.2, 1.0 Hz, 1H), |
| | | | | | 0.81 (dq, J = 7.5, 2.3 Hz, 3H). |
| 393 | (2R,3S,4S,5R)-3-(3,4- | 512.429 | 513.22 | 3.50 | ¹H NMR (400 MHZ, DMSO- |
| | difluoro-2- | | | | d₆) δ 10.81-10.76 (m, 1H), |
| | methoxyphenyl)-4,5- | | | | 9.01-8.95 (m, 1H), 8.36 (dd, J = |
| | dimethyl-N-(6-(3- | | | | 8.7, 2.5 Hz, 1H), 8.23-8.16 |
| | methyl-1,2,4- | | | | (m, 1H), 7.24-7.12 (m, 2H), |
| | oxadiazol-5- | | | | 5.16 (d, J = 10.2 Hz, 1H), 4.29 |
| | yl)pyridin-3-yl)-5- | | | | (dd, J = 10.2, 7.7 Hz, 1H), 3.96 |
| | (trifluoromethyl)- | | | | (d, J = 2.2 Hz, 3H), 2.79 (p, J = |
| | tetrahydrofuran-2- | | | | 7.5 Hz, 1H), 2.43 (s, 3H), 1.63 |
| | carboxamide | | | | (s, 3H), 0.78-0.71 (m, 3H) |
| | | | | | ppm. |
| 394 | (2R,3S,4S,5R)-3-(3,4- | 497.415 | 498.12 | 3.37 | ¹H NMR (400 MHZ, DMSO- |
| | difluoro-2- | | | | d₆) δ 10.57 (s, 1H), 8.83 (dd, J = |
| | methoxyphenyl)-4,5- | | | | 2.5, 0.8 Hz, 1H), 8.49 (s, |
| | dimethyl-N-(6- | | | | 1H), 8.20 (dd, J = 8.7, 2.5 Hz, |
| | (oxazol-5-yl)pyridin- | | | | 1H), 7.78-7.68 (m, 2H), 7.24- |
| | 3-yl)-5- | | | | 7.11 (m, 2H), 5.12 (d, J = |
| | (trifluoromethyl)- | | | | 10.2 Hz, 1H), 4.27 (dd, J = |
| | tetrahydrofuran-2- | | | | 10.3, 7.7 Hz, 1H), 3.96 (d, J = |
| | carboxamide | | | | 2.2 Hz, 3H), 2.78 (p, J = 7.3 |
| | | | | | Hz, 1H), 1.62 (s, 3H), 0.74 (dt, |
| | | | | | J = 7.7, 2.5 Hz, 3H) ppm. |
| 395 | (2R,3S,4S,5R)-3-(3,4- | 510.456 | 511.2 | 3.74 | ¹H NMR (400 MHZ, DMSO- |
| | difluoro-2- | | | | d₆) δ 10.48 (s, 1H), 8.63 (dd, J = |
| | methoxyphenyl)-4,5- | | | | 2.6, 0.7 Hz, 1H), 8.42 (d, J = |
| | dimethyl-N-(6-(3- | | | | 2.5 Hz, 1H), 8.19 (dd, J = 8.9, |
| | methyl-1H-pyrazol-1- | | | | 2.6 Hz, 1H), 7.82 (dd, J = 8.9, |
| | yl)pyridin-3-yl)-5- | | | | 0.7 Hz, 1H), 7.24-7.15 (m, |
| | (trifluoromethyl)- | | | | 1H), 7.16 (s, 1H), 6.35 (d, J = |
| | tetrahydrofuran-2- | | | | 2.5 Hz, 1H), 5.09 (dd, J = 17.8, |
| | carboxamide | | | | 10.4 Hz, 1H), 4.26 (dt, J = |
| | | | | | 10.5, 7.2 Hz, 1H), 3.96 (t, J = |
| | | | | | 2.4 Hz, 3H), 2.78 (p, J = 7.3 |
| | | | | | Hz, 1H), 2.28 (s, 3H), 1.61 (d, |
| | | | | | J = 8.2 Hz, 3H), 0.74 (d, J = |
| | | | | | 6.9 Hz, 3H) ppm. |
| 396 | (2R,3S,4S,5R)-N- | 507.452 | 508.17 | 3.59 | ¹H NMR (400 MHZ, DMSO- |
| | ([2,2′-bipyridin]-5-yl)- | | | | d₆) δ 10.58 (s, 1H), 8.88 (dd, J = |
| | 3-(3,4-difluoro-2- | | | | 2.6, 0.8 Hz, 1H), 8.65 (ddd, J = |
| | methoxyphenyl)-4,5- | | | | 4.8, 1.8, 0.9 Hz, 1H), 8.39- |
| | dimethyl-5- | | | | 8.28 (m, 2H), 8.22 (dd, J = 8.7, |
| | (trifluoromethyl)- | | | | 2.5 Hz, 1H), 7.92 (td, J = 7.7, |
| | tetrahydrofuran-2- | | | | 1.8 Hz, 1H), 7.41 (ddd, J = 7.5, |
| | carboxamide | | | | 4.8, 1.2 Hz, 1H), 7.25-7.13 |
| | | | | | (m, 2H), 5.14 (d, J = 10.3 Hz, |
| | | | | | 1H), 4.29 (dd, J = 10.3, 7.6 Hz, |
| | | | | | 1H), 3.97 (d, J = 2.2 Hz, 3H), |
| | | | | | 2.79 (p, J = 7.5 Hz, 1H), 1.63 |
| | | | | | (s, 3H), 0.75 (dt, J = 7.7, 2.5 |
| | | | | | Hz, 3H) ppm. |
| 397 | (2R,3S,4S,5R)-3-(3,4- | 511.444 | 510.12 | 2.48 | ¹H NMR (400 MHZ, DMSO- |
| | difluoro-2- | | | | d₆) δ 10.63 (s, 1H), 8.91 (dd, J = |
| | methoxyphenyl)-4,5- | | | | 2.5, 0.8 Hz, 1H), 8.58 (s, |
| | dimethyl-N-(6-(4- | | | | 1H), 8.22 (dd, J = 8.7, 2.5 Hz, |
| | methyl-4H-1,2,4- | | | | 1H), 8.10 (dd, J = 8.7, 0.8 Hz, |
| | triazol-3-yl)pyridin-3- | | | | 1H), 7.25-7.13 (m, 2H), 5.14 |
| | yl)-5- | | | | (d, J = 10.2 Hz, 1H), 4.28 (dd, |
| | (trifluoromethyl)- | | | | J = 10.2, 7.6 Hz, 1H), 3.96 (d, |
| | tetrahydrofuran-2- | | | | J = 2.5 Hz, 6H), 2.79 (p, J = |
| | carboxamide | | | | 7.5 Hz, 1H), 1.62 (s, 3H), 0.75 |
| | | | | | (dd, J = 7.5, 2.6 Hz, 3H) ppm. |
| 398 | (2R,3S,4S,5R)-3-(3,4- | 512.429 | 513.12 | 3.31 | ¹H NMR (400 MHZ, DMSO- |
| | difluoro-2- | | | | d6) δ 10.72 (s, 1H), 8.95 (dd, J = |
| | methoxyphenyl)-4,5- | | | | 2.5, 0.8 Hz, 1H), 8.30 (dd, J = |
| | dimethyl-N-(6-(5- | | | | 8.7, 2.5 Hz, 1H), 8.11 (dd, J = |
| | methyl-1,3,4- | | | | 8.6, 0.7 Hz, 1H), 7.24-7.12 |
| | oxadiazol-2- | | | | (m, 2H), 5.15 (d, J = 10.2 Hz, |
| | yl)pyridin-3-yl)-5- | | | | 1H), 4.28 (dd, J = 10.2, 7.6 Hz, |
| | (trifluoromethyl)- | | | | 1H), 3.96 (d, J = 2.2 Hz, 3H), |
| | tetrahydrofuran-2- | | | | 2.79 (p, J = 7.5 Hz, 1H), 2.59 |
| | carboxamide | | | | (s, 3H), 1.62 (s, 3H), 0.74 (dt, |
| | | | | | J = 7.6, 2.5 Hz, 3H) ppm. |
| 399 | (2R,3S,4S,5R)-3-(3,4- | 497.415 | 498.12 | 3.36 | ¹H NMR (400 MHZ, DMSO- |
| | difluoro-2- | | | | d₆) δ 10.65 (s, 1H), 8.90 (dd, J = |
| | methoxyphenyl)-4,5- | | | | 2.5, 0.8 Hz, 1H), 8.29-8.22 |
| | dimethyl-N-(6- | | | | (m, 2H), 8.07 (dd, J = 8.6, 0.7 |
| | (oxazol-2-yl)pyridin- | | | | Hz, 1H), 7.42 (d, J = 0.8 Hz, |
| | 3-yl)-5- | | | | 1H), 7.24-7.12 (m, 2H), 5.14 |
| | (trifluoromethyl)- | | | | (d, J = 10.3 Hz, 1H), 4.28 (dd, |
| | tetrahydrofuran-2- | | | | J = 10.2, 7.7 Hz, 1H), 3.96 (d, |
| | carboxamide | | | | J = 2.2 Hz, 3H), 2.79 (p, J = |
| | (TFA salt) | | | | 7.5 Hz, 1H), 1.62 (s, 3H), 0.74 |
| | | | | | (dt, J = 7.4, 2.4 Hz, 3H) ppm. |
| 400 | (2R,3S,4S,5R)-3-(3,4- | 510.456 | 511.17 | 3.36 | ¹H NMR (400 MHZ, DMSO- |
| | difluoro-2- | | | | d₆) δ 10.46 (s, 1H), 8.73 (dd, J = |
| | methoxyphenyl)-4,5- | | | | 2.6, 0.8 Hz, 1H), 8.08 (dd, J = |
| | dimethyl-N-(6-(1- | | | | 8.6, 2.6 Hz, 1H), 7.86 (td, J = |
| | methyl-1H-pyrazol-3- | | | | 8.9, 1.2 Hz, 1H), 7.74 (d, J = |
| | yl)pyridin-3-yl)-5- | | | | 2.2 Hz, 1H), 7.53-7.44 (m, |
| | (trifluoromethyl)- | | | | 1H), 7.37 (d, J = 1.9 Hz, 1H), |
| | tetrahydrofuran-2- | | | | 7.23-7.17 (m, 1H), 6.73 (d, J = |
| | carboxamide | | | | 2.2 Hz, 1H), 5.11 (d, J = |
| | | | | | 10.3 Hz, 1H), 4.27 (dd, J = |
| | | | | | 10.3, 7.6 Hz, 1H), 3.96 (d, J = |
| | | | | | 2.1 Hz, 3H), 3.89 (s, 3H), 2.78 |
| | | | | | (p, J = 7.4 Hz, 1H), 1.62 (s, |
| | | | | | 3H), 0.74 (dt, J = 7.3, 2.5 Hz, |
| | | | | | 3H) ppm. |
| 401 | (2R,3S,4S,5R)-N-(6- | 496.43 | 497.12 | 3.34 | ¹H NMR (400 MHZ, DMSO- |
| | (1H-imidazol-1- | | | | d₆) δ 10.54 (s, 1H), 8.69 (dd, J = |
| | yl)pyridin-3-yl)-3- | | | | 2.6, 0.7 Hz, 1H), 8.46 (t, J = |
| | (3,4-difluoro-2- | | | | 1.1 Hz, 1H), 8.23 (dd, J = 8.9, |
| | methoxyphenyl)-4,5- | | | | 2.6 Hz, 1H), 7.90 (t, J = 1.4 |
| | dimethyl-5- | | | | Hz, 1H), 7.78 (dd, J = 8.9, 0.7 |
| | (trifluoromethyl)- | | | | Hz, 1H), 7.24-7.13 (m, 2H), |
| | tetrahydrofuran-2- | | | | 7.11 (t, J = 1.2 Hz, 1H), 5.12 |
| | carboxamide | | | | (d, J = 10.3 Hz, 1H), 4.27 (dd, |
| | | | | | J = 10.2, 7.6 Hz, 1H), 3.96 (d, |
| | | | | | J = 2.2 Hz, 3H), 2.78 (p, J = |
| | | | | | 7.4 Hz, 1H), 1.62 (s, 3H), 0.78- |
| | | | | | 0.71 (m, 3H) ppm. |
| 402 | (2R,3S,4S,5R)-N-(6- | 497.418 | 498.1 | 3.4 | ¹H NMR (400 MHZ, DMSO- |
| | (1H-1,2,4-triazol-1- | | | | d₆) δ 10.61 (s, 1H), 9.31 (s, |
| | yl)pyridin-3-yl)-3- | | | | 1H), 8.76 (dd, J = 2.6, 0.7 Hz, |
| | (3,4-difluoro-2- | | | | 1H), 8.31 (dd, J = 8.9, 2.6 Hz, |
| | methoxyphenyl)-4,5- | | | | 1H), 8.28 (s, 1H), 7.89-7.82 |
| | dimethyl-5- | | | | (m, 1H), 7.24-7.12 (m, 2H), |
| | (trifluoromethyl)- | | | | 5.13 (d, J = 10.3 Hz, 1H), 4.28 |
| | tetrahydrofuran-2- | | | | (dd, J = 10.3, 7.7 Hz, 1H), 3.96 |
| | carboxamide | | | | (d, J = 2.2 Hz, 3H), 2.79 (p, J = |
| | (TFA salt) | | | | 7.5 Hz, 1H), 1.62 (s, 3H), 0.74 |
| | | | | | (dt, J = 7.2, 2.4 Hz, 3H) ppm. |
| 403 | (2R,3S,4S,5R)-3-(3,4- | 484.416 | 485.17 | 3.27 | 1H NMR (400 MHZ, |
| | difluoro-2- | | | | Chloroform-d) δ 8.59 (dd, J = |
| | methoxyphenyl)-N-(6- | | | | 2.7, 0.7 Hz, 1H), 8.44 (s, 1H), |
| | (3-hydroxyprop-1-yn- | | | | 8.18 (dd, J = 8.6, 2.6 Hz, 1H), |
| | 1-yl)pyridin-3-yl)-4,5- | | | | 7.42 (d, J = 8.6 Hz, 1H), 7.11 |
| | dimethyl-5- | | | | (ddd, J = 8.1, 5.5, 2.1 Hz, 1H), |
| | (trifluoromethyl)- | | | | 6.93 (td, J = 9.2, 7.4 Hz, 1H), |
| | tetrahydrofuran-2- | | | | 5.05 (d, J = 11.0 Hz, 1H), 4.53 |
| | carboxamide ( | | | | (d, J = 4.2 Hz, 2H), 4.11 (dd, J = |
| | TFA salt) | | | | 11.0, 8.1 Hz, 1H), 4.03 (d, J = |
| | | | | | 2.8 Hz, 3H), 2.78 (p, J = 7.6 |
| | | | | | Hz, 1H), 1.76 (s, 1H), 0.90 (s, |
| | | | | | 1H), 0.81 (dt, J = 7.4, 2.4 Hz, |
| | | | | | 3H) ppm. |
| 404 | (2R,3S,4S,5R)-3-(3,4- | 528.511 | 529 | 3.48 | ¹H NMR (400 MHZ, DMSO- |
| | difluoro-2- | | | | d₆) δ 10.40 (s, 1H), 8.72 (d, J = |
| | methoxyphenyl)-4,5- | | | | 2.6 Hz, 1H), 8.00 (d, J = 8.7 |
| | dimethyl-N-(6- | | | | Hz, 1H), 7.27 (d, J = 8.4 Hz, |
| | ((tetrahydro-2H-pyran- | | | | 1H), 7.23-7.14 (m, 1H), 7.16 |
| | 4-yl)methyl)pyridin-3- | | | | (d, J = 5.4 Hz, 1H), 5.09 (d, J = |
| | yl)-5- | | | | 10.4 Hz, 1H), 4.24 (dd, J = |
| | (trifluoromethyl)- | | | | 10.4, 7.6 Hz, 1H), 3.95 (d, J = |
| | tetrahydrofuran-2- | | | | 2.2 Hz, 3H), 3.83-3.75 (m, |
| | carboxamide | | | | 2H), 3.27-3.17 (m, 2H), 2.76 |
| | | | | | (p, J = 7.6 Hz, 1H), 2.64 (d, J = |
| | | | | | 7.1 Hz, 2H), 1.96-1.86 (m, |
| | | | | | 1H), 1.60 (s, 3H), 1.47-1.39 |
| | | | | | (m, 2H), 1.21 (qd, J = 11.8, 4.3 |
| | | | | | Hz, 2H), 0.76-0.70 (m, 3H) |
| | | | | | ppm. |

The following compound was made using a method similar to that described in Example 10, except that 1-(4-bromo-2-pyridyl)-2-methyl-propan-2-ol was used in step 2 with alternative C-N coupling conditions (10 mol % o t-BuBrettphos-Pd-G3 and 1.5 equivalents of potassium phosphate in t-BuOH at 100° C. for 1.5 hours under a nitrogen atmosphere). In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 405 | (2R,3S,4S,5R)-3- | 502.474 | 504 | 3.44 | ¹H NMR (500 MHZ, DMSO-d₆) |
| | (3,4-difluoro-2- | | | | δ 10.56 (s, 1H), 8.36 (d, J = 5.8 |
| | methoxyphenyl)-N- | | | | Hz, 1H), 7.57 (s, 1H), 7.51 (s, |
| | (2-(2-hydroxy-2- | | | | 1H), 7.23-7.05 (m, 2H), 5.09 |
| | methylpropyl)- | | | | (d, J = 10.3 Hz, 1H), 4.78 (s, |
| | pyridin-4-yl)-4,5- | | | | 1H), 4.25 (dd, J = 10.3, 7.7 Hz, |
| | dimethyl-5- | | | | 1H), 3.95 (d, J = 2.0 Hz, 3H), |
| | (trifluoromethyl)- | | | | 2.83-2.70 (m, 3H), 1.59 (s, |
| | tetrahydrofuran-2- | | | | 3H), 1.08 (d, J = 2.7 Hz, 6H), |
| | carboxamide | | | | 0.72 (d, J = 7.3 Hz, 3H) ppm. |

The following compounds were made using a method similar to that described in Example 10, except that 2-[1-(azetidin-1-yl)-2-methoxy-ethyl]-5-bromo-pyridine was used in step 2 with 2-methyl tetrahydrofuran as solvent. Diastereomers generated in step 2 were separated by chiral SFC. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 406 | rel- | 543.526 | 544.3 | 3.46 | ¹H NMR (500 MHz, DMSO- |
| | (2R*,3S*,4S*,5R*)- | | | | d₆) δ 10.35 (s, 1H), 8.65 (s, |
| | N-(6-(1-(azetidin-1- | | | | 1H), 7.97 (d, J = 8.5 Hz, 1H), |
| | yl)-2- | | | | 7.30 (d, J = 8.5 Hz, 1H), 7.22- |
| | methoxyethyl)- | | | | 7.11 (m, 2H), 5.08 (d, J = 10.3 |
| | pyridin-3-yl)-3-(3,4- | | | | Hz, 1H), 4.23 (dd, J = 10.3, 7.6 |
| | difluoro-2- | | | | Hz, 1H), 3.95 (d, J = 2.1 Hz, |
| | methoxyphenyl)-4,5- | | | | 3H), 3.58-3.36 (m, 3H), 3.15 |
| | dimethyl-5- | | | | (s, 3H), 3.13-2.97 (m, 4H), |
| | (trifluoromethyl)- | | | | 2.76 (p, J = 7.5 Hz, 1H), 1.92 |
| | tetrahydrofuran-2- | | | | (s, 2H), 1.60 (s, 3H), 0.78- |
| | carboxamide | | | | 0.67 (m, 3H) ppm. |
| | (first eluting isomer | | | | |
| | by SFC using | | | | |
| | Chiralpak ID | | | | |
| | column) | | | | |
| 407 | rel- | 543.526 | 544.3 | 3.44 | ¹H NMR (500 MHZ, DMSO- |
| | (2R*,3S*,4S*,5R*)- | | | | d₆) δ 10.35 (s, 1H), 8.65 (d, J = |
| | N-(6-(1-(azetidin-1- | | | | 2.5 Hz, 1H), 7.96 (dd, J = 8.5, |
| | yl)-2- | | | | 2.6 Hz, 1H), 7.30 (d, J = 8.5 |
| | methoxyethyl)- | | | | Hz, 1H), 7.22-7.09 (m, 2H), |
| | pyridin-3-yl)-3- | | | | 5.08 (d, J = 10.4 Hz, 1H), 4.23 |
| | (3,4-difluoro-2- | | | | (dd, J = 10.3, 7.6 Hz, 1H), 3.95 |
| | methoxyphenyl)- | | | | (d, J = 2.0 Hz, 3H), 3.54-3.37 |
| | 4,5-dimethyl-5- | | | | (m, 3H), 3.15 (s, 3H), 3.13- |
| | (trifluoromethyl)- | | | | 2.96 (m, 4H), 2.76 (p, J = 7.5 |
| | tetrahydrofuran-2- | | | | Hz, 1H), 1.97-1.85 (m, 2H), |
| | carboxamide | | | | 1.60 (s, 3H), 0.73 (d, J = 6.9 |
| | (second eluting | | | | Hz, 3H). |
| | isomer by SFC using | | | | |
| | Chiralpak ID | | | | |
| | column) | | | | |

The following compounds were made using a method similar to that described in Example 10, except that 6-bromo-2,3-dihydrofuro[3,2-b]pyridin-3-ol was used in step 2 with t-BuBrettphos-Pd-G3 and potassium carbonate in dioxane at 90′° C. Diastereomers generated in step 2 were separated by chiral SFC. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 408 | rel- | 488.405 | 489.5 | 3.23 | ¹H NMR (400 MHZ, DMSO- |
| | (2R*,3S*,4S*,5R*)-3- | | | | d₆) δ 10.41 (s, 1H), 8.27 (d, J = |
| | (3,4-difluoro-2- | | | | 2.0 Hz, 1H), 7.58 (d, J = 2.0 |
| | methoxyphenyl)-N- | | | | Hz, 1H), 7.21-7.10 (m, 2H), |
| | (3-hydroxy-2,3- | | | | 5.78 (d, J = 4.6 Hz, 1H), 5.13- |
| | dihydrofuro[3,2- | | | | 5.10 (m, 1H), 5.08 (d, J = 10.3 |
| | b]pyridin-6-yl)-4,5- | | | | Hz, 1H), 4.62 (dd, J = 10.4, 6.9 |
| | dimethyl-5- | | | | Hz, 1H), 4.31 (dd, J = 10.4, 2.9 |
| | (trifluoromethyl)- | | | | Hz, 1H), 4.23 (dd, J = 10.3, 7.5 |
| | tetrahydrofuran-2- | | | | Hz, 1H), 3.94 (d, J = 2.2 Hz, |
| | carboxamide | | | | 3H), 2.76 (dq, J = 7.5, 7.5 Hz, |
| | (first eluting isomer by | | | | 1H), 1.59 (s, 3H), 0.75-0.70 |
| | SFC using Chiralpak | | | | (m, 3H) ppm. |
| | IC column) | | | | |
| 409 | rel- | 488.405 | 489.5 | 3.23 | ¹H NMR (400 MHZ, DMSO- |
| | (2R*,3S*,4S*,5R*)-3- | | | | d₆) δ 10.43 (s, 1H), 8.27 (d, J = |
| | (3,4-difluoro-2- | | | | 2.0 Hz, 1H), 7.58 (d, J = 2.0 |
| | methoxyphenyl)-N- | | | | Hz, 1H), 7.21-7.11 (m, 2H), |
| | (3-hydroxy-2,3- | | | | 5.80 (s, 1H), 5.12-5.09 (m, |
| | dihydrofuro[3,2- | | | | 1H), 5.08 (d, J = 10.3 Hz, 1H), |
| | b]pyridin-6-yl)-4,5- | | | | 4.61 (dd, J = 10.4, 6.9 Hz, 1H), |
| | dimethyl-5- | | | | 4.31 (dd, J = 10.4, 2.9 Hz, 1H), |
| | (trifluoromethyl)- | | | | 4.23 (dd, J = 10.3, 7.6 Hz, 1H), |
| | tetrahydrofuran-2- | | | | 3.94 (d, J = 2.2 Hz, 3H), 2.76 |
| | carboxamide | | | | (dq, J = 7.5, 7.5 Hz, 1H), 1.59 |
| | (second eluting isomer | | | | (s, 3H), 0.75-0.69 (m, 3H) |
| | by SFC using | | | | ppm. |
| | Chiralpak IC column) | | | | |

The following compounds were made using a method similar to that described in Example 10, except that different coupling partners were used in step 2 and General Method I was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 410 | (2R,3S,4S,5R)-3-(3,4- | 545.542 | | 3.57 | ¹H NMR (500 MHZ, DMSO- |
| | difluoro-2- | | | | d₆) δ 10.54 (s, 1H), 8.33 (d, J = |
| | methoxyphenyl)-N- | | | | 5.5 Hz, 1H), 7.63 (d, J = 2.0 |
| | (2-(((1-methoxy-2- | | | | Hz, 1H), 7.50 (dd, J = 5.6, 2.1 |
| | methylpropan-2- | | | | Hz, 1H), 7.22-7.08 (m, 2H), |
| | yl)amino)methyl)- | | | | 5.08 (d, J = 10.3 Hz, 1H), 4.24 |
| | pyridin-4-yl)-4,5- | | | | (dd, J = 10.3, 7.6 Hz, 1H), 3.95 |
| | dimethyl-5- | | | | (d, J = 2.0 Hz, 3H), 3.71 (s, |
| | (trifluoromethyl)- | | | | 2H), 3.26 (s, 3H), 3.17 (s, 2H), |
| | tetrahydrofuran-2- | | | | 2.77 (p, J = 7.5 Hz, 1H), 1.59 |
| | carboxamide | | | | (s, 3H), 1.02 (s, 6H), 0.73 (d, J = |
| | | | | | 7.6 Hz, 3H) ppm. |
| 411 | (2R,3S,4S,5R)-3-(3,4- | 528.472 | 529 | 3.03 | ¹H NMR (400 MHZ, DMSO- |
| | difluoro-2- | | | | d₆) δ 10.67 (s, 1H), 8.30 (dd, J = |
| | methoxyphenyl)-4,5- | | | | 5.6, 0.6 Hz, 1H), 8.13-8.07 |
| | dimethyl-N-(2-(2- | | | | (m, 1H), 7.52 (dd, J = 5.6, 1.9 |
| | oxopiperazin-1- | | | | Hz, 1H), 7.21-7.07 (m, 2H), |
| | yl)pyridin-4-yl)-5- | | | | 5.09 (d, J = 10.2 Hz, 1H), 4.25 |
| | (trifluoromethyl)- | | | | (dd, J = 10.2, 7.6 Hz, 1H), 3.95 |
| | tetrahydrofuran-2- | | | | (d, J = 2.2 Hz, 3H), 3.78 (q, J = |
| | carboxamide | | | | 5.4 Hz, 2H), 3.43 (s, 2H), 2.99 |
| | | | | | (s, 2H), 2.81-2.70 (m, 2H), |
| | | | | | 1.60 (s, 3H), 0.73 (d, J = 7.0 |
| | | | | | Hz, 3H) ppm. |

The following compounds were made using a method similar to that described in Example 10, except that different coupling partners were used in step 2. Diastereomers generated in step 2 were separated by chiral SFC and each isomer was submitted to General Method I as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 412 | rel- | 517.489 | 518.4 | 2.88 | ¹H NMR (500 MHz, DMSO- |
| | (2R*,3S*,4S*,5R*)-3- | | | | d₆) δ 10.53 (s, 1H), 8.37 (d, J = |
| | (3,4-difluoro-2- | | | | 5.6 Hz, 1H), 7.67 (d, J = 2.0 |
| | methoxyphenyl)-N-(2- | | | | Hz, 1H), 7.49 (dd, J = 5.6, 2.1 |
| | (2-methoxy-1- | | | | Hz, 1H), 7.21-7.09 (m, 2H), |
| | (methylamino)ethyl)- | | | | 5.08 (d, J = 10.3 Hz, 1H), 4.25 |
| | pyridin-4-yl)-4,5- | | | | (dd, J = 10.3, 7.7 Hz, 1H), 3.95 |
| | dimethyl-5- | | | | (d, J = 2.0 Hz, 3H), 3.71 (dd, J = |
| | (trifluoromethyl)- | | | | 7.4, 5.2 Hz, 1H), 3.46 (dd, J = |
| | tetrahydrofuran-2- | | | | 9.6, 5.2 Hz, 1H), 3.38 (dd, J = |
| | carboxamide | | | | 9.5, 7.4 Hz, 1H), 3.21 (s, |
| | (precursor was first | | | | 3H), 2.77 (p, J = 7.5 Hz, 1H), |
| | eluting isomer by SFC | | | | 2.17 (s, 3H), 1.60 (s, 3H), 0.72 |
| | using Lux Cellulose-2 | | | | (dd, J = 7.6, 2.4 Hz, 3H) ppm. |
| | column) | | | | |
| 413 | rel- | 517.489 | 518.4 | 2.89 | ¹H NMR (500 MHZ, DMSO- |
| | (2R*,3S*,4S*,5R*)-3- | | | | d₆) δ 10.54 (s, 1H), 8.37 (d, J = |
| | (3,4-difluoro-2- | | | | 5.6 Hz, 1H), 7.65 (d, J = 2.1 |
| | methoxyphenyl)-N-(2- | | | | Hz, 1H), 7.51 (dd, J = 5.6, 2.1 |
| | (2-methoxy-1- | | | | Hz, 1H), 7.23-7.07 (m, 2H), |
| | (methylamino)ethyl)- | | | | 5.08 (d, J = 10.3 Hz, 1H), 4.25 |
| | pyridin-4-yl)-4,5- | | | | (dd, J = 10.3, 7.6 Hz, 1H), 3.95 |
| | dimethyl-5- | | | | (d, J = 2.0 Hz, 3H), 3.70 (dd, J = |
| | (trifluoromethyl)- | | | | 7.5, 5.2 Hz, 1H), 3.46 (dd, J = |
| | tetrahydrofuran-2- | | | | 9.5, 5.2 Hz, 1H), 3.38 (dd, J = |
| | carboxamide | | | | 9.6, 7.4 Hz, 1H), 3.21 (s, |
| | (precursor was second | | | | 3H), 2.77 (p, J = 7.5 Hz, 1H), |
| | eluting isomer by SFC | | | | 2.17 (s, 3H), 1.60 (s, 3H), 0.72 |
| | using Lux Cellulose-2 | | | | (dd, J = 7.5, 2.4 Hz, 3H) ppm. |
| | column) | | | | |
| 414 | rel- | 517.489 | 518.3 | 2.88 | ¹H NMR (500 MHZ, DMSO- |
| | (2R*,3S*,4S*,5R*)-3- | | | | d₆) δ 10.35 (s, 1H), 8.68 (d, J = |
| | (3,4-difluoro-2- | | | | 2.5 Hz, 1H), 7.98 (dd, J = 8.5, |
| | methoxyphenyl)-N-(6- | | | | 2.6 Hz, 1H), 7.37 (d, J = 8.5 |
| | (2-methoxy-1- | | | | Hz, 1H), 7.21-7.12 (m, 2H), |
| | (methylamino)ethyl)- | | | | 5.09 (d, J = 10.3 Hz, 1H), 4.24 |
| | pyridin-3-yl)-4,5- | | | | (dd, J = 10.3, 7.7 Hz, 1H), 3.95 |
| | dimethyl-5- | | | | (d, J = 2.0 Hz, 3H), 3.75-3.69 |
| | (trifluoromethyl)- | | | | (m, 1H), 3.47-3.37 (m, 2H), |
| | tetrahydrofuran-2- | | | | 3.20 (s, 3H), 2.76 (p, J = 7.5 |
| | carboxamide | | | | Hz, 1H), 2.14 (d, J = 1.9 Hz, |
| | (precursor was first | | | | 3H), 1.60 (s, 3H), 0.76-0.68 |
| | eluting isomer by SFC | | | | (m, 3H) ppm. |
| | using Lux Cellulose-2 | | | | |
| | column) | | | | |
| 415 | rel- | 517.489 | 518.3 | 2.88 | ¹H NMR (500 MHZ, DMSO- |
| | (2R*,3S*,4S*,5R*)-3- | | | | d₆) δ 10.36 (s, 1H), 8.68 (d, J = |
| | (3,4-difluoro-2- | | | | 2.6 Hz, 1H), 7.98 (dd, J = 8.5, |
| | methoxyphenyl)-N-(6- | | | | 2.5 Hz, 1H), 7.37 (d, J = 8.5 |
| | (2-methoxy-1- | | | | Hz, 1H), 7.22-7.09 (m, 2H), |
| | (methylamino)ethyl)- | | | | 5.09 (d, J = 10.3 Hz, 1H), 4.24 |
| | pyridin-3-yl)-4,5- | | | | (dd, J = 10.3, 7.6 Hz, 1H), 3.95 |
| | dimethyl-5- | | | | (d, J = 2.0 Hz, 3H), 3.73 (t, J = |
| | (trifluoromethyl)- | | | | 6.3 Hz, 1H), 3.48-3.37 (m, |
| | tetrahydrofuran-2- | | | | 2H), 3.20 (s, 3H), 2.76 (p, J = |
| | carboxamide | | | | 7.6 Hz, 1H), 2.15 (s, 3H), 1.60 |
| | (precursor was second | | | | (s, 3H), 0.78-0.68 (m, 3H) |
| | eluting isomer by SFC | | | | ppm. |
| | using Lux Cellulose-2 | | | | |
| | column) | | | | |
| 416 | rel- | 561.541 | 562.3 | 2.92 | ¹H NMR (500 MHZ, DMSO- |
| | (2R*,3S*,4S*,5R*)-3- | | | | d₆) δ 10.37 (s, 1H), 8.69 (d, J = |
| | (3,4-difluoro-2- | | | | 2.5 Hz, 1H), 7.98 (dd, J = 8.5, |
| | methoxyphenyl)-N-(6- | | | | 2.6 Hz, 1H), 7.39 (d, J = 8.5 |
| | (2-methoxy-1-((2- | | | | Hz, 1H), 7.21-7.11 (m, 2H), |
| | methoxyethyl)amino)- | | | | 5.09 (d, J = 10.3 Hz, 1H), 4.24 |
| | ethyl)pyridin-3-yl)- | | | | (dd, J = 10.3, 7.6 Hz, 1H), 3.95 |
| | 4,5-dimethyl-5- | | | | (d, J = 2.0 Hz, 3H), 3.93-3.83 |
| | (trifluoromethyl)- | | | | (m, 1H), 3.49-3.34 (m, 4H), |
| | tetrahydrofuran-2- | | | | 3.20 (d, J = 5.1 Hz, 6H), 2.76 |
| | carboxamide | | | | (p, J = 7.6 Hz, 1H), 2.60-2.53 |
| | (precursor was first | | | | (m, 1H), 2.49-2.44 (m, 1H), |
| | eluting isomer by SFC | | | | 1.60 (s, 3H), 0.73 (dd, J = 7.4, |
| | using OD-H column) | | | | 2.5 Hz, 3H) ppm. |
| 417 | rel- | 561.541 | 562 | 2.92 | ¹H NMR (500 MHz, DMSO- |
| | (2R*,3S*,4S*,5R*)-3- | | | | d₆) δ 10.38 (s, 1H), 8.69 (d, J = |
| | (3,4-difluoro-2- | | | | 2.5 Hz, 1H), 7.98 (dd, J = 8.5, |
| | methoxyphenyl)-N-(6- | | | | 2.5 Hz, 1H), 7.39 (d, J = 8.4 |
| | (2-methoxy-1-((2- | | | | Hz, 1H), 7.22-7.11 (m, 2H), |
| | methoxyethyl)amino)- | | | | 5.09 (d, J = 10.3 Hz, 1H), 4.24 |
| | ethyl)pyridin-3-yl)- | | | | (dd, J = 10.3, 7.6 Hz, 1H), 3.95 |
| | 4,5-dimethyl-5- | | | | (d, J = 2.0 Hz, 4H), 3.49-3.33 |
| | (trifluoromethyl)- | | | | (m, 4H), 3.20 (d, J = 4.8 Hz, |
| | tetrahydrofuran-2- | | | | 6H), 2.76 (p, J = 7.6 Hz, 1H), |
| | carboxamide | | | | 2.57 (s, 1H), 2.47 (d, J = 5.6 |
| | (precursor was second | | | | Hz, 1H), 1.60 (s, 3H), 0.76- |
| | eluting isomer by SFC | | | | 0.69 (m, 3H) ppm. |
| | using OD-H column) | | | | |
| 418 | rel- | 535.479 | | 3.535 | ¹H NMR (500 MHz, DMSO- |
| | (2R*,3S*,4S*,5R*)-3- | | | | d₆) δ 10.28 (s, 1H), 8.51 (s, |
| | (3,4-difluoro-2- | | | | 1H), 8.15 (d, J = 6.3 Hz, 1H), |
| | methoxyphenyl)-N-(5- | | | | 7.23-7.14 (m, 2H), 5.38- |
| | fluoro-2-(2-methoxy-1- | | | | 5.31 (m, 1H), 4.25 (dd, J = |
| | (methylamino)ethyl)- | | | | 10.5, 7.5 Hz, 1H), 3.95 (d, J = |
| | pyridin-4-yl)-4,5- | | | | 2.1 Hz, 3H), 3.92-3.78 (m, |
| | dimethyl-5- | | | | 1H), 3.51-3.39 (m, 2H), 3.21 |
| | (trifluoromethyl)- | | | | (s, 3H), 2.77 (p, J = 7.6 Hz, |
| | tetrahydrofuran-2- | | | | 1H), 2.19 (s, 3H), 1.60 (s, 3H), |
| | carboxamide | | | | 0.73 (d, J = 7.4 Hz, 3H) ppm. |
| | (precursor was first | | | | |
| | eluting isomer by SFC | | | | |
| | using Chiralpak IG | | | | |
| | column) | | | | |
| 419 | rel- | 535.479 | 536.2 | 3.23 | ¹H NMR (500 MHz, DMSO- |
| | (2R*,3S*,4S*,5R*)-3- | | | | d₆) δ 10.28 (s, 1H), 8.52-8.47 |
| | (3,4-difluoro-2- | | | | (m, 1H), 8.18-8.10 (m, 1H), |
| | methoxyphenyl)-N-(5- | | | | 7.23-7.13 (m, 2H), 5.34 (d, J = |
| | fluoro-2-(2-methoxy- | | | | 10.4 Hz, 1H), 4.25 (dd, J = |
| | 1-(methylamino)ethyl)- | | | | 10.5, 7.5 Hz, 1H), 3.95 (d, J = |
| | pyridin-4-yl)-4,5- | | | | 2.1 Hz, 3H), 3.86-3.72 (m, |
| | dimethyl-5- | | | | 1H), 3.49-3.35 (m, 2H), 3.20 |
| | (trifluoromethyl)- | | | | (s, 3H), 2.77 (p, J = 7.5 Hz, |
| | tetrahydrofuran-2- | | | | 1H), 2.17 (s, 3H), 1.61 (s, 3H), |
| | carboxamide | | | | 0.72 (d, J = 7.4 Hz, 3H) ppm. |
| | (precursor was second | | | | |
| | eluting isomer by SFC | | | | |
| | using Chiralpak IG | | | | |
| | column) | | | | |
| 420 | rel- | 545.542 | 546.2 | 3.54 | ¹H NMR (500 MHZ, |
| | (2R*,3S*,4S*,5R*)-3- | | | | Methanol-d₄) δ 8.72 (d, J = 2.4 |
| | (3,4-difluoro-2- | | | | Hz, 1H), 8.03 (dd, J = 8.5, 2.5 |
| | methoxyphenyl)-N-(6- | | | | Hz, 1H), 7.45 (dd, J = 8.5, 0.7 |
| | (1-(isopropylamino)-2- | | | | Hz, 1H), 7.13 (ddd, J = 8.2, |
| | methoxyethyl)pyridin- | | | | 5.6, 2.1 Hz, 1H), 7.01-6.94 |
| | 3-yl)-4,5-dimethyl-5- | | | | (m, 1H), 5.08 (d, J = 10.5 Hz, |
| | (trifluoromethyl)- | | | | 1H), 4.31 (dd, J = 10.5, 8.0 Hz, |
| | tetrahydrofuran-2- | | | | 1H), 4.16-4.06 (m, 1H), 3.99 |
| | carboxamide | | | | (d, J = 2.2 Hz, 3H), 3.58-3.51 |
| | (precursor was first | | | | (m, 2H), 3.29 (s, 3H), 2.79 (p, |
| | eluting isomer by SFC | | | | J = 7.6 Hz, 1H), 2.69-2.58 |
| | using Chiralpak IG | | | | (m, 1H), 1.66 (s, 3H), 1.05 (d, |
| | column) | | | | J = 6.2 Hz, 3H), 1.01 (d, J = |
| | | | | | 6.4 Hz, 3H), 0.85-0.78 (m, |
| | | | | | 3H) ppm. |
| 421 | rel- | 545.542 | | 3.55 | ¹H NMR (500 MHZ, |
| | (2R*,3S*,4S*,5R*)-3- | | | | Methanol-d₄) δ 8.73 (d, J = 2.4 |
| | (3,4-difluoro-2- | | | | Hz, 1H), 8.04 (dd, J = 8.5, 2.5 |
| | methoxyphenyl)-N-(6- | | | | Hz, 1H), 7.45 (d, J = 8.5 Hz, |
| | (1-(isopropylamino)-2- | | | | 1H), 7.13 (ddd, J = 8.2, 5.6, |
| | methoxyethyl)pyridin- | | | | 2.1 Hz, 1H), 7.05-6.94 (m, |
| | 3-yl)-4,5-dimethyl-5- | | | | 1H), 5.08 (d, J = 10.4 Hz, 1H), |
| | (trifluoromethyl)- | | | | 4.31 (dd, J = 10.5, 8.0 Hz, 1H), |
| | tetrahydrofuran-2- | | | | 4.17-4.09 (m, 1H), 3.99 (d, J = |
| | carboxamide | | | | 2.1 Hz, 3H), 3.59-3.51 (m, |
| | (precursor was second | | | | 2H), 3.30 (s, 3H), 2.79 (p, J = |
| | eluting isomer by SFC | | | | 7.6 Hz, 1H), 2.72-2.61 (m, |
| | using Chiralpak IG | | | | 1H), 1.68-1.64 (m, 3H), 1.06 |
| | column) | | | | (d, J = 6.2 Hz, 3H), 1.02 (d, J = |
| | | | | | 6.4 Hz, 3H), 0.85-0.77 (m, |
| | | | | | 3H) ppm. |
| 422 | rel- | 521.453 | 522.6 | 3.4 | ¹H NMR (500 MHZ, DMSO- |
| | (2R*,3S*,4S*,5R*)-N- | | | | d₆) δ 10.24 (s, 1H), 8.42 (s, |
| | (2-(1-amino-2- | | | | 1H), 8.13 (d, J = 6.4 Hz, 1H), |
| | methoxyethyl)-5- | | | | 7.23-7.08 (m, 2H), 5.30 (m, |
| | fluoropyridin-4-yl)-3- | | | | 1H), 4.23 (dd, J = 10.4, 7.5 Hz, |
| | (3,4-difluoro-2- | | | | 1H), 4.01-3.89 (m, 4H), 3.46 |
| | methoxyphenyl)-4,5- | | | | (dd, J = 9.2, 5.4 Hz, 1H), 3.39- |
| | dimethyl-5- | | | | 3.32 (m, 1H), 3.20 (s, 3H), |
| | (trifluoromethyl)- | | | | 2.81-2.71 (m, 1H), 1.89 (s, |
| | tetrahydrofuran-2- | | | | 2H), 1.60 (s, 3H), 0.72 (d, J = |
| | carboxamide | | | | 7.3 Hz, 3H) ppm. |
| | (precursor was first | | | | |
| | eluting isomer by SFC | | | | |
| | using Chiralpak ID | | | | |
| | column) | | | | |
| 423 | rel- | 521.453 | 522.6 | 3.39 | ¹H NMR (500 MHZ, DMSO- |
| | (2R*,3S*,4S*,5R*)-N- | | | | d₆) δ 10.24 (s, 1H), 8.43 (s, |
| | (2-(1-amino-2- | | | | 1H), 8.13 (d, J = 6.4 Hz, 1H), |
| | methoxyethyl)-5- | | | | 7.21-7.14 (m, 2H), 5.32- |
| | fluoropyridin-4-yl)-3- | | | | 5.28 (m, 1H), 4.23 (dd, J = |
| | (3,4-difluoro-2- | | | | 10.4, 7.5 Hz, 1H), 4.01-3.93 |
| | methoxyphenyl)-4,5- | | | | (m, 4H), 3.46 (dd, J = 9.3, 5.5 |
| | dimethyl-5- | | | | Hz, 1H), 3.37-3.31 (m, 1H), |
| | (trifluoromethyl)- | | | | 3.20 (s, 3H), 2.76 (p, J = 7.4 |
| | tetrahydrofuran-2- | | | | Hz, 1H), 1.89 (s, 2H), 1.60 (s, |
| | carboxamide | | | | 3H), 0.75-0.69 (m, 3H) ppm. |
| | (precursor was second | | | | |
| | eluting isomer by SFC | | | | |
| | using Chiralpak ID | | | | |
| | column) | | | | |

The following compound was made using a method similar to that described in Example 10, except that tert-butyl N-[(4-bromo-2-pyridyl)methyl]-N-(2-methoxy-1,1-dimethyl-ethyl)carbamate was used in step 2. The product from step 2 was deprotected using General Method I and methylated using General Method K. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 15 | (2R,3S,4S,5R)-3-(3,4- | 559.569 | 560.1 | 2.97 | ¹H NMR (500 MHz, DMSO-d₆) |
| | difluoro-2- | | | | δ 10.59 (s, 1H), 8.30 (s, 1H), |
| | methoxyphenyl)-N-(2- | | | | 7.61 (s, 1H), 7.55 (dd, J = 5.6, |
| | (((1-methoxy-2- | | | | 2.1 Hz, 1H), 7.17 (td, J = 9.5, |
| | methylpropan-2- | | | | 7.5 Hz, 1H), 7.13-7.07 (m, |
| | yl)(methyl)amino)- | | | | 1H), 5.08 (d, J = 10.3 Hz, 1H), |
| | methyl)pyridin-4-yl)- | | | | 4.25 (dd, J = 10.3, 7.7 Hz, 1H), |
| | 4,5-dimethyl-5- | | | | 3.95 (d, J = 2.1 Hz, 3H), 3.64 (s, |
| | (trifluoromethyl)- | | | | 2H), 3.29-3.23 (m, 6H), 2.77 |
| | tetrahydrofuran-2- | | | | (p, J = 7.5 Hz, 1H), 2.10 (s, 2H), |
| | carboxamide | | | | 1.59 (s, 3H), 1.07 (s, 6H), 0.76- |
| | | | | | 0.68 (m, 3H) ppm. |

The following compounds were made using a method similar to that described in Example 10, except that different coupling partners were used in step 2 with 2-methyl tetrahydrofuran as solvent. The diastereomers generated in step 2 were separated by chiral SFC and the separated isomers then deprotected using General Method I and methylated using General Method K. In the Table below, “-MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 424 | rel- | 531.515 | 532.4 | 2.92 | ¹H NMR (500 MHz, DMSO-d₆) |
| | (2R*,3S*,4S*,5R*)-3- | | | | δ 10.54 (s, 1H), 8.38 (d, J = 5.5 |
| | (3,4-difluoro-2- | | | | Hz, 1H), 7.62 (s, 1H), 7.51 (dd, |
| | methoxyphenyl)-N-(2- | | | | J = 5.6, 2.1 Hz, 1H), 7.21-7.09 |
| | (1-(dimethylamino)-2- | | | | (m, 2H), 5.08 (d, J = 10.3 Hz, |
| | methoxyethyl)pyridin- | | | | 1H), 4.25 (dd, J = 10.3, 7.7 Hz, |
| | 4-yl)-4,5-dimethyl-5- | | | | 1H), 3.95 (d, J = 2.1 Hz, 3H), |
| | (trifluoromethyl)- | | | | 3.72 (dd, J = 10.0, 6.0 Hz, 1H), |
| | tetrahydrofuran-2- | | | | 3.67 (t, J = 7.7 Hz, 1H), 3.56 (s, |
| | carboxamide | | | | 1H), 3.18 (s, 3H), 2.77 (p, J = |
| | (precursor was first | | | | 7.6 Hz, 1H), 2.18 (s, 6H), 1.60 |
| | eluting isomer by SFC | | | | (s, 3H), 0.77-0.67 (m, 3H) |
| | using Lux Cellulose-2 | | | | ppm. |
| | column) | | | | |
| 425 | rel- | 531.515 | 532.4 | 2.92 | ¹H NMR (500 MHZ, DMSO-d₆) |
| | (2R*,3S*,4S*,5R*)-3- | | | | δ 10.61 (s, 1H), 8.38 (d, J = 5.5 |
| | (3,4-difluoro-2- | | | | Hz, 1H), 7.65 (s, 1H), 7.51 (dd, |
| | methoxyphenyl)-N-(2- | | | | J = 5.6, 2.1 Hz, 1H), 7.20-7.11 |
| | (1-(dimethylamino)-2- | | | | (m, 2H), 5.10 (d, J = 10.3 Hz, |
| | methoxyethyl)pyridin- | | | | 1H), 4.25 (dd, J = 10.4, 7.6 Hz, |
| | 4-yl)-4,5-dimethyl-5- | | | | 1H), 3.95 (d, J = 2.0 Hz, 3H), |
| | (trifluoromethyl)- | | | | 3.76-3.52 (m, 3H), 3.18 (s, |
| | tetrahydrofuran-2- | | | | 3H), 2.76 (p, J = 7.5 Hz, 1H), |
| | carboxamide | | | | 2.21 (s, 6H), 1.60 (s, 3H), 0.72 |
| | (precursor was second | | | | (d, J = 6.3 Hz, 3H) ppm. |
| | eluting isomer by SFC | | | | |
| | using Lux Cellulose-2 | | | | |
| | column) | | | | |
| 426 | rel- | 531.515 | 532.3 | 2.90 | ¹H NMR (500 MHZ, DMSO-d₆) |
| | (2R*,3S*,4S*,5R*)-3- | | | | δ 10.39 (s, 1H), 8.70 (d, J = 2.5 |
| | (3,4-difluoro-2- | | | | Hz, 1H), 7.99 (dd, J = 8.5, 2.6 |
| | methoxyphenyl)-N-(6- | | | | Hz, 1H), 7.33 (d, J = 8.5 Hz, |
| | (1-(dimethylamino)-2- | | | | 1H), 7.25-7.10 (m, 2H), 5.09 |
| | methoxyethyl)pyridin- | | | | (d, J = 10.3 Hz, 1H), 4.24 (dd, J = |
| | 3-yl)-4,5-dimethyl-5- | | | | 10.3, 7.6 Hz, 1H), 3.95 (d, J = |
| | (trifluoromethyl)- | | | | 2.0 Hz, 3H), 3.80-3.62 (m, |
| | tetrahydrofuran-2- | | | | 3H), 3.18 (s, 3H), 2.76 (p, J = |
| | carboxamide | | | | 7.5 Hz, 1H), 2.19 (s, 6H), 1.60 |
| | (precursor was first | | | | (s, 3H), 0.73 (dd, J = 7.5, 2.4 |
| | eluting isomer by SFC | | | | Hz, 3H) ppm. |
| | using Lux Cellulose-2 | | | | |
| | column) | | | | |
| 427 | rel- | 531.515 | 532.3 | 2.92 | ¹H NMR (500 MHz, DMSO-d₆) |
| | (2R*,3S*,4S*,5R*)-3- | | | | δ 10.40 (s, 1H), 8.70 (s, 1H), |
| | (3,4-difluoro-2- | | | | 8.00 (d, J = 8.4 Hz, 1H), 7.34 (d, |
| | methoxyphenyl)-N-(6- | | | | J = 8.5 Hz, 1H), 7.22-7.12 (m, |
| | (1-(dimethylamino)-2- | | | | 2H), 5.09 (d, J = 10.4 Hz, 1H), |
| | methoxyethyl)pyridin- | | | | 4.25 (dd, J = 10.3, 7.6 Hz, 1H), |
| | 3-yl)-4,5-dimethyl-5- | | | | 3.95 (d, J = 2.0 Hz, 3H), 3.80- |
| | (trifluoromethyl)- | | | | 3.55 (m, 3H), 3.19 (s, 3H), 2.76 |
| | tetrahydrofuran-2- | | | | (p, J = 7.4 Hz, 1H), 2.19 (s, 6H), |
| | carboxamide | | | | 1.60 (s, 3H), 0.73 (d, J = 7.3 Hz, |
| | (precursor was second | | | | 3H) ppm. |
| | eluting isomer by SFC | | | | |
| | using Lux Cellulose-2 | | | | |
| | column) | | | | |

The following compound was made using a method similar to that described in Example 10, except that tert-butyl N-[2-[tert-butyl(dimethyl)silyl]oxy-1-(4-chloro-5-fluoro-2-pyridyl)-2-methyl-propyl]carbamate was used as coupling partner in step 2 with (5-diphenylphosphanyl-9,9-dimethyl-xanthen-4-yl)-diphenyl-phosphane; [2-[2-(methylamino)phenyl]phenyl]-methylsulfonyloxy-palladium in place of Pd(OAc)₂/Xantphos. The diastereomers generated in step 2 were separated by chiral SFC and the separated isomers then deprotected using General Method I and General Method J. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 428 | rel-(2R*,3S*,4S*,5R*)- | 535.479 | 537.2 | 3.36 | ¹H NMR (500 MHZ, DMSO- |
| | N-(2-(1-amino-2- | | | | d₆) δ 10.22 (s, 1H), 8.43 (s, |
| | hydroxy-2- | | | | 1H), 8.08 (s, 1H), 7.19 (d, J = |
| | methylpropyl)-5- | | | | 4.2 Hz, 2H), 5.31 (s, 1H), 4.49 |
| | fluoropyridin-4-yl)-3- | | | | (s, 1H), 4.24 (dd, J = 10.5, 7.6 |
| | (3,4-difluoro-2- | | | | Hz, 1H), 3.95 (s, 3H), 3.65 (s, |
| | methoxyphenyl)-4,5- | | | | 1H), 3.17 (s, 1H), 2.77 (p, J = |
| | dimethyl-5- | | | | 7.4 Hz, 1H), 1.93 (s, 1H), 1.61 |
| | (trifluoromethyl)- | | | | (s, 3H), 1.00 (s, 3H), 0.95 (d, J = |
| | tetrahydrofuran- | | | | 6.0 Hz, 3H), 0.73 (d, J = 7.5 |
| | 2-carboxamide | | | | Hz, 3H) ppm. |
| | (precursor was first | | | | |
| | eluting isomer by SFC | | | | |
| | using Chiralpak IA | | | | |
| | column) | | | | |
| 429 | rel-(2R*,3S*,4S*,5R*)- | 535.479 | 537.1 | 3.37 | ¹H NMR (500 MHZ, DMSO- |
| | N-(2-(1-amino-2- | | | | d₆) δ 10.44 (s, 1H), 8.63 (s, |
| | hydroxy-2- | | | | 1H), 8.24-8.16 (m, 3H), 7.20 |
| | methylpropyl)-5- | | | | (s, 2H), 5.36 (d, J = 10.4 Hz, |
| | fluoropyridin-4-yl)-3- | | | | 1H), 5.26 (s, 1H), 4.29-4.22 |
| | (3,4-difluoro-2- | | | | (m, 2H), 3.96 (s, 3H), 2.79 (p, |
| | methoxyphenyl)-4,5- | | | | J = 7.5 Hz, 1H), 1.61 (s, 3H), |
| | dimethyl-5- | | | | 1.14 (s, 3H), 1.02 (s, 3H), 0.74 |
| | (trifluoromethyl)- | | | | (d, J = 7.5 Hz, 3H) ppm. |
| | tetrahydrofuran- | | | | |
| | 2-carboxamide | | | | |
| | (precursor was second | | | | |
| | eluting isomer by SFC | | | | |
| | using Chiralpak IA | | | | |
| | column) | | | | |

The following compound was made using a method similar to that described in Example 10, except that 7-bromo-2-(2-triisopropylsilyloxyethyl)-[1,2,4]triazolo[4,3-a]pyridin-3-one was used as coupling partner in step 2 with t-BuBrettphos-Pd-G3 and potassium phosphate in t-BuOH at 80° C. The product from step 2 was deprotected using General Method J. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 430 | (2R,3S,4S,5R)-3-(3,4- | 530.445 | 531.6 | 3.12 | ¹H NMR (400 MHZ, DMSO- |
| | difluoro-2- | | | | d₆) δ 10.51 (s, 1H), 7.84 (dd, J = |
| | methoxyphenyl)-N-(2- | | | | 7.6, 0.9 Hz, 1H), 7.62 (dd, J = |
| | (2-hydroxyethyl)-3- | | | | 1.9, 1.0 Hz, 1H), 7.22-7.15 |
| | oxo-2,3-dihydro- | | | | (m, 1H), 7.12 (t, J = 7.5 Hz, |
| | [1,2,4]triazolo[4,3- | | | | 1H), 6.68 (dd, J = 7.6, 1.9 Hz, |
| | a]pyridin-7-yl)-4,5- | | | | 1H), 5.07 (d, J = 10.2 Hz, 1H), |
| | dimethyl-5- | | | | 4.77 (t, J = 5.8 Hz, 1H), 4.26 |
| | (trifluoromethyl)- | | | | (dd, J = 10.2, 7.7 Hz, 1H), 3.96 |
| | tetrahydrofuran-2- | | | | (d, J = 2.2 Hz, 3H), 3.87 (t, J = |
| | carboxamide | | | | 5.8 Hz, 2H), 3.69 (q, J = 5.8 |
| | | | | | Hz, 2H), 2.78 (p, J = 7.5 Hz, |
| | | | | | 1H), 1.60 (s, 3H), 0.73 (d, J = |
| | | | | | 7.4 Hz, 3H). |

The following compound was made using a method similar to that described in Example 10, except that ethyl 6-bromo-[1,2,4]triazolo[1,5-a]pyridine-2-carboxylate was used as coupling partner in step 2 with t-BuBrettphos-Pd-G3 and potassium phosphate in t-BuOH at 80° C. General method L was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 16 | 6-((2R,3S,4S,5R)-3- | 513.417 | 514 | 3.12 | ¹H NMR (500 MHz, DMSO- |
| | (3,4-difluoro-2- | | | | d₆) δ 10.65 (s, 1H), 9.42 (dd, |
| | methoxyphenyl)-4,5- | | | | J = 2.0, 0.9 Hz, 1H), 8.03 (s, |
| | dimethyl-5- | | | | 1H), 7.88 (d, J = 9.6 Hz, 1H), |
| | (trifluoromethyl)- | | | | 7.79 (dd, J = 9.6, 2.0 Hz, |
| | tetrahydrofuran-2- | | | | 1H), 7.74 (s, 1H), 7.18 (t, J = |
| | carboxamido)- | | | | 5.4 Hz, 2H), 5.13 (d, J = 10.3 |
| | [1,2,4]triazolo[1,5- | | | | Hz, 1H), 4.28 (dd, J = 10.2, |
| | a]pyridine-2- | | | | 7.8 Hz, 1H), 3.96 (d, J = 2.0 |
| | carboxamide | | | | Hz, 3H), 2.79 (p, J = 7.6 Hz, |
| | | | | | 1H), 1.62 (s, 3H), 0.74 (d, J = |
| | | | | | 7.3 Hz, 3H) ppm. |

The following compounds were made using a method similar to that described in Example 10, except that a different coupling partners were used in step 2 with tBuBrettphos Pd G3 and potassium carbonate at 90° C. General method M was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 431 | (2R,3S,4S,5R)-3-(3,4- | 500.419 | 501.5 | 3.04 | ¹H NMR (400 MHz, DMSO- |
| | difluoro-2- | | | | d₆) δ 10.62 (s, 1H), 8.40 (dd, |
| | methoxyphenyl)-N-(3- | | | | J = 7.5, 0.9 Hz, 1H), 8.11 |
| | (hydroxymethyl)- | | | | (dd, J = 2.0, 0.9 Hz, 1H), |
| | [1,2,4]triazolo[4,3- | | | | 7.17 (dd, J = 9.8, 6.4 Hz, |
| | α]pyridin-7-yl)-4,5- | | | | 2H), 7.11 (dd, J = 7.5, 2.0 |
| | dimethyl-5- | | | | Hz, 1H), 5.66 (t, J = 5.7 Hz, |
| | (trifluoromethyl)tetrahydrofuran- | | | | 1H), 5.11 (d, J = 10.2 Hz, |
| | 2-carboxamide | | | | 1H), 4.92 (d, J = 5.7 Hz, 2H), |
| | | | | | 4.28 (dd, J = 10.2, 7.7 Hz, |
| | | | | | 1H), 3.96 (d, J = 2.2 Hz, 3H), |
| | | | | | 2.79 (dq, J = 7.5, 7.5 Hz, |
| | | | | | 1H), 1.61 (s, 3H), 0.74 (d, J = |
| | | | | | 6.0 Hz, 3H) ppm. |
| 432 | (2R,3S,4S,5R)-3-(3,4- | 500.419 | 501.5 | 3.07 | ¹H NMR (400 MHz, DMSO- |
| | difluoro-2- | | | | d₆) δ 10.53 (s, 1H), 9.16 (dd, |
| | methoxyphenyl)-N-(3- | | | | J = 1.9, 1.0 Hz, 1H), 7.78 |
| | (hydroxymethyl)- | | | | (dd, J = 9.8, 1.0 Hz, 1H), |
| | [1,2,4]triazolo[4,3- | | | | 7.43 (dd, J = 9.8, 1.9 Hz, |
| | α]pyridin-6-yl)-4,5- | | | | 1H), 7.22-7.12 (m, 2H), |
| | dimethyl-5- | | | | 5.66 (t, J = 5.6 Hz, 1H), 5.13 |
| | (trifluoromethyl)tetrahydrofuran- | | | | (d, J = 10.4 Hz, 1H), 4.92 (d, |
| | 2-carboxamide | | | | J = 5.6 Hz, 2H), 4.28 (dd, J = |
| | | | | | 10.4, 7.5 Hz, 1H), 3.97 (d, J = |
| | | | | | 2.2 Hz, 3H), 2.79 (dq, J = |
| | | | | | 7.5, 7.5 Hz, 1H), 1.62 (s, |
| | | | | | 3H), 0.74 (d, J = 6.0 Hz, 3H) |
| | | | | | ppm. |

The following compounds were made using a method similar to that described in Example 10, except that (3-bromo-6,7-dihydro-SH-cyclopenta[b]pyridin-7-yl)oxy-tert-butyl-dimethyl-silane was used in step 2. The diastereomers generated in step 2 were separated by chiral SFC and the separated isomers then deprotected using General Method M. In the Table below, “MS r.t.” stands for Mass Spec

| Cmpd | | LC/MS | Found | MS | |
|---|---|---|---|---|---|
| No. | Compound Name | (m/z calc.) | M + 1 | r.t. | NMR (shifts in ppm) |
| 433 | rel-(2R*,3S*,4S*,5R*)- | 486.432 | 487.6 | 3.26 | ¹H NMR (500 MHz, DMSO- |
| | 3-(3,4-difluoro-2- | | | | d₆) δ 10.36 (s, 1H), 8.55 (d, J = |
| | methoxyphenyl)-N-(7- | | | | 2.2 Hz, 1H), 7.93 (d, J = |
| | hydroxy-6,7-dihydro- | | | | 2.2 Hz, 1H), 7.21-7.11 (m, |
| | 5H-cyclopenta[b]pyridin- | | | | 2H), 5.09 (d, J = 10.3 Hz, |
| | 3-yl)-4,5-dimethyl-5- | | | | 1H), 4.89 (dd, J = 7.2, 5.1 |
| | (trifluoromethyl)tetrahydrofuran- | | | | Hz, 1H), 4.24 (dd, J = 10.3, |
| | 2-carboxamide | | | | 7.6 Hz, 1H), 3.94 (d, J = 2.0 |
| | (precursor was first | | | | Hz, 3H), 2.95-2.87 (m, 1H), |
| | eluting isomer by SFC | | | | 2.79-2.65 (m, 2H), 2.38- |
| | using Amylose SA | | | | 2.28 (m, 1H), 1.87-1.77 (m, |
| | column) | | | | 1H), 1.60 (s, 3H), 0.73 (d, J = |
| | | | | | 6.8 Hz, 3H) ppm. |
| 17 | rel-(2R*,3S*,4S*,5R*)- | 486.432 | 487.6 | 3.25 | ¹H NMR (500 MHz, DMSO- |
| | 3-(3,4-difluoro-2- | | | | d₆) δ 10.32 (s, 1H), 8.54 (d, J = |
| | methoxyphenyl)-N-(7- | | | | 2.2 Hz, 1H), 7.89 (d, J = |
| | hydroxy-6,7-dihydro- | | | | 2.2 Hz, 1H), 7.20-7.11 (m, |
| | 5H-cyclopenta[b]pyridin- | | | | 2H), 5.25 (d, J = 5.5 Hz, 1H), |
| | 3-yl)-4,5-dimethyl-5- | | | | 5.08 (d, J = 10.3 Hz, 1H), |
| | (trifluoromethyl)tetrahydrofuran- | | | | 4.89-4.84 (m, 1H), 4.23 (dd, |
| | 2-carboxamide | | | | J = 10.3, 7.7 Hz, 1H), 3.94 |
| | (precursor was second | | | | (d, J = 2.0 Hz, 3H), 2.94- |
| | eluting isomer by SFC | | | | 2.86 (m, 1H), 2.79-2.66 (m, |
| | using Amylose SA | | | | 2H), 2.37-2.28 (m, 1H), |
| | column) | | | | 1.85-1.77 (m, 1H), 1.60 (s, |
| | | | | | 3H), 0.73 (d, J = 6.3 Hz, 3H) |
| | | | | | ppm. |

The following compounds were made using a method similar to that described in Example 10, except that (3-bromo-6,7-dihydro-5H-cyclopenta[b]pyridin-5-yl)oxy-tert-butyl-dimethyl-silane was used in step 2 with t-BuBrettphos Pd G3 and potassium carbonate in dioxane at 90° C. The diastereomers generated in step 2 were separated by chiral SFC and the separated isomers then deprotected using General Method M. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 434 | rel-(2R*,3S*,4S*,5R*)- | 486.432 | 487.6 | 3.14 | ¹H NMR (400 MHz, DMSO- |
| | 3-(3,4-difluoro-2- | | | | d₆) δ 10.34 (s, 1H), 8.50 (d, J = |
| | methoxyphenyl)-N-(5- | | | | 2.4 Hz, 1H), 8.00 (d, J = |
| | hydroxy-6,7-dihydro- | | | | 2.4 Hz, 1H), 7.21-7.11 (m, |
| | 5H-cyclopenta[b]pyridin- | | | | 2H), 5.42 (d, J = 5.3 Hz, 1H), |
| | 3-yl)-4,5-dimethyl-5- | | | | 5.08 (d, J = 10.3 Hz, 1H), |
| | (trifluoromethyl)tetrahydrofuran- | | | | 5.05-5.01 (m, 1H), 4.24 (dd, |
| | 2-carboxamide | | | | J = 10.3, 7.6 Hz, 1H), 3.95 |
| | (precursor was first | | | | (d, J = 2.2 Hz, 3H), 2.87 |
| | eluting isomer by SFC | | | | (ddd, J = 16.5, 9.0, 3.8 Hz, |
| | using Chiralpak IG | | | | 1H), 2.80-2.67 (m, 2H), |
| | column) | | | | 2.43-2.33 (m, 1H), 1.83- |
| | | | | | 1.73 (m, 1H), 1.60 (s, 3H), |
| | | | | | 0.74-0.70 (m, 3H) ppm. |
| 435 | rel-(2R*,3S*,4S*,5R*)- | 486.432 | 487.5 | 3.14 | ¹H NMR (400 MHz, DMSO- |
| | 3-(3,4-difluoro-2- | | | | d₆) δ 10.33 (s, 1H), 8.51 (d, J = |
| | methoxyphenyl)-N-(5- | | | | 2.4 Hz, 1H), 7.99 (d, J = |
| | hydroxy-6,7-dihydro- | | | | 2.4 Hz, 1H), 7.21-7.11 (m, |
| | 5H-cyclopenta[b]pyridin- | | | | 2H), 5.43 (d, J = 5.6 Hz, 1H), |
| | 3-yl)-4,5-dimethyl-5- | | | | 5.08 (d, J = 10.3 Hz, 1H), |
| | (trifluoromethyl)tetrahydrofuran- | | | | 5.06-5.00 (m, 1H), 4.24 (dd, |
| | 2-carboxamide | | | | J = 10.3, 7.6 Hz, 1H), 3.94 |
| | (precursor was second | | | | (d, J = 2.1 Hz, 3H), 2.87 |
| | eluting isomer by SFC | | | | (ddd, J = 16.4, 9.0, 3.8 Hz, |
| | using Chiralpak IG | | | | 1H), 2.79-2.66 (m, 2H), |
| | column) | | | | 2.43-2.33 (m, 1H), 1.84- |
| | | | | | 1.73 (m, 1H), 1.60 (s, 3H), |
| | | | | | 0.76-0.70 (m, 3H) ppm. |

The following compounds were made using a method similar to that described in Example 10, except that different aryl bromides were used in step 2 with t-BuBrettphos Pd G3 and potassium phosphate in t-BuOH at 100° C. The diastereomers generated in step 2 were separated by chiral SFC and then deprotected using General Method M as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 436 | rel- | 544.511 | 546 | 3.73 | ¹H NMR (400 MHz, DMSO- |
| | (2R*,3S*,4S*,5R*)--3- | | | | d₆) δ 10.46 (s, 1H), 7.98 (d, J = |
| | (3,4-difluoro-2- | | | | 5.8 Hz, 1H), 7.24-7.09 (m, |
| | methoxyphenyl)-N-(2- | | | | 3H), 7.06 (dd, J = 5.7, 1.8 Hz, |
| | (((1R,2S)-2- | | | | 1H), 5.07 (d, J = 10.2 Hz, 1H), |
| | hydroxycyclohexyl)oxy)pyridin- | | | | 5.01 (dt, J = 8.3, 2.7 Hz, 1H), |
| | 4-yl)-4,5-dimethyl-5- | | | | 4.43 (s, 1H), 4.24 (dd, J = |
| | (trifluoromethyl)tetrahydrofuran- | | | | 10.2, 7.6 Hz, 1H), 3.94 (d, J = |
| | 2-carboxamide | | | | 2.2 Hz, 3H), 3.78 (dd, J = 6.7, |
| | (precursor was first | | | | 3.5 Hz, 1H), 2.76 (p, J = 7.5 |
| | eluting isomer by SFC | | | | Hz, 1H), 1.84 (p, J = 8.1, 7.4 |
| | using Whelk-O1 | | | | Hz, 1H), 1.74-1.63 (m, 1H), |
| | column) | | | | 1.59 (s, 4H), 1.55-1.45 (m, |
| | | | | | 3H), 1.30 (tq, J = 9.5, 5.7, 4.5 |
| | | | | | Hz, 2H), 0.72 (dt, J = 7.4, 2.4 |
| | | | | | Hz, 3H) ppm. |
| 437 | rel- | 532.457 | 533 | 3.16 | ¹H NMR (400 MHz, DMSO- |
| | (2R*,3S*,4S*,5R*)--3- | | | | d₆) δ 10.38 (s, 1H), 8.36 (d, J = |
| | (3,4-difluoro-2- | | | | 2.0 Hz, 1H), 8.07 (d, J = 2.6 |
| | methoxyphenyl)-N-(5- | | | | Hz, 1H), 7.77 (dd, J = 2.7, 2.1 |
| | (((3R,4S)-4- | | | | Hz, 1H), 7.25-7.07 (m, 2H), |
| | hydroxytetrahydrofuran- | | | | 5.12 (d, J = 6.2 Hz, 1H), 5.09 |
| | 3-yl)oxy)pyridin-3- | | | | (d, J = 10.3 Hz, 1H), 4.79 (td, J = |
| | yl)-4,5-dimethyl-5- | | | | 5.3, 4.3 Hz, 1H), 4.41-4.34 |
| | (trifluoromethyl)tetrahydrofuran- | | | | (m, 1H), 4.25 (dd, J = 10.3, 7.6 |
| | 2-carboxamide | | | | Hz, 1H), 4.03 (dd, J = 9.6, 5.5 |
| | (precursor was first | | | | Hz, 1H), 3.95 (d, J = 2.2 Hz, |
| | eluting isomer by SFC | | | | 3H), 3.89 (dd, J = 8.7, 5.9 Hz, |
| | using Whelk-O1 | | | | 1H), 3.73 (dd, J = 9.6, 4.2 Hz, |
| | column) | | | | 1H), 3.56 (dd, J = 8.7, 5.8 Hz, |
| | | | | | 1H), 2.77 (p, J = 7.5 Hz, 1H), |
| | | | | | 1.60 (s, 3H), 0.80-0.64 (m, |
| | | | | | 3H) ppm. |
| 438 | rel- | 492.411 | 494 | 3.28 | |
| | (2R*,3S*,4S*,5R*)-3- | | | | |
| | (3,4-difluoro-2- | | | | |
| | methoxyphenyl)-N-(6- | | | | |
| | (1-fluoro-2- | | | | |
| | hydroxyethyl)pyridin- | | | | |
| | 3-yl)-4,5-dimethyl-5- | | | | |
| | (trifluoromethyl)tetrahydrofuran- | | | | |
| | 2-carboxamide | | | | |
| | (precursor was second | | | | |
| | eluting isomer by SFC | | | | |
| | using Lux Cellulose-2 | | | | |
| | column) | | | | |

The following compound was made using a method similar to that described in Example 10, except that 2-(2-tert-butoxy-1-fluoro-ethyl)-4-chloro-pyridine was used as the coupling partner in step 2 with t-BuBrettphos Pd G3 and potassium phosphate in t-BuOH. The diastereomers generated in step 2 were separated by chiral SFC and the separated isomers then deprotected using 4M HCl in dioxane as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 439 | rel-(2R*,3S*,4S*,5R*)- | 492.411 | 493.3 | 3.30 | ¹H NMR (400 MHz, DMSO- |
| | 3-(3,4-difluoro-2- | | | | d₆) δ 10.63 (s, 1H), 8.42 (d, J = |
| | methoxyphenyl)-N-(2- | | | | 5.5 Hz, 1H), 7.74 (t, J = 1.5 |
| | (1-fluoro-2- | | | | Hz, 1H), 7.58 (dd, J = 5.6, |
| | hydroxyethyl)pyridin- | | | | 2.1 Hz, 1H), 7.24-7.10 (m, |
| | 4-yl)-4,5-dimethyl-5- | | | | 2H), 5.46 (ddd, J = 48.7, 6.3, |
| | (trifluoromethyl)tetrahydrofuran- | | | | 2.8 Hz, 1H), 5.15-5.05 (m, |
| | 2-carboxamide | | | | 2H), 4.25 (dd, J = 10.3, 7.7 |
| | (precursor was first | | | | Hz, 1H), 3.95 (d, J = 2.2 Hz, |
| | eluting isomer by SFC | | | | 3H), 3.86-3.56 (m, 2H), |
| | using Lux Cellulose-5 | | | | 2.77 (p, J = 7.5 Hz, 1H), 1.60 |
| | column) | | | | (s, 3H), 0.76-0.68 (m, 3H) |
| | | | | | ppm. |
| 440 | rel-(2R*,3S*,4S*,5R*)- | 492.411 | | 3.31 | ¹H NMR (400 MHz, DMSO- |
| | 3-(3,4-difluoro-2- | | | | d₆) δ 10.62 (s, 1H), 8.42 (d, J = |
| | methoxyphenyl)-N-(2- | | | | 5.5 Hz, 1H), 7.73 (t, J = 1.5 |
| | (1-fluoro-2- | | | | Hz, 1H), 7.58 (dd, J = 5.6, |
| | hydroxyethyl)pyridin- | | | | 2.1 Hz, 1H), 7.22-7.06 (m, |
| | 4-yl)-4,5-dimethyl-5- | | | | 2H), 5.45 (ddd, J = 48.6, 6.3, |
| | (trifluoromethyl)tetrahydrofuran- | | | | 2.8 Hz, 1H), 5.15-5.04 (m, |
| | 2-carboxamide | | | | 2H), 4.25 (dd, J = 10.3, 7.7 |
| | (precursor was second | | | | Hz, 1H), 3.95 (d, J = 2.2 Hz, |
| | eluting isomer by SFC | | | | 3H), 3.92-3.64 (m, 2H), |
| | using Lux Cellulose-5 | | | | 2.77 (t, J = 7.5 Hz, 1H), 1.60 |
| | column) | | | | (s, 3H), 0.73 (dd, J = 7.7, 2.3 |
| | | | | | Hz, 3H) ppm. |

The following compound was made from 440 using General Method A. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 441 | rel- | 508.411 | 509.2 | 3.03 | ¹H NMR (500 MHz, DMSO- |
| | 4-((2R*,3S*,4S*,5R*)- | | | | d₆) δ 10.70 (s, 1H), 8.19 (dd, |
| | 3-(3,4-difluoro-2- | | | | J = 7.2, 1.0 Hz, 1H), 7.83 |
| | methoxyphenyl)-4,5- | | | | (dd, J = 2.9, 1.2 Hz, 1H), |
| | dimethyl-5- | | | | 7.69 (dd, J = 7.2, 3.1 Hz, |
| | (trifluoromethyl)tetrahydrofuran- | | | | 1H), 7.25-7.04 (m, 2H), |
| | 2-carboxamido)-2-(1- | | | | 5.86 (ddd, J = 46.8, 4.7, 2.3 |
| | fluoro-2- | | | | Hz, 1H), 5.21 (t, J = 6.1 Hz, |
| | hydroxyethyl)pyridine | | | | 1H), 5.07 (d, J = 10.3 Hz, |
| | 1-oxide | | | | 1H), 4.25 (dd, J = 10.3, 7.7 |
| | | | | | Hz, 1H), 3.95 (d, J = 2.1 Hz, |
| | | | | | 3H), 3.92-3.67 (m, 2H), |
| | | | | | 2.76 (t, J = 7.5 Hz, 1H), 1.60 |
| | | | | | (s, 3H), 0.79-0.63 (m, 3H) |
| | | | | | ppm. |

The following compounds were made using a method similar to that described in Example 10, except that different coupling partners were used in step 2 and reduction using General Method P was carried out as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 442 | ((2R,3S,4S,5R)-3-(3,4- | 500.419 | 501.2 | 3.10 | ¹H NMR (500 MHz, |
| | difluoro-2- | | | | Methanol-d₄) δ 9.35-9.27 (m, |
| | methoxyphenyl)-N-(2- | | | | 1H), 7.62 (dd, J = 9.5, 2.0 Hz, |
| | (hydroxymethyl)- | | | | 1H), 7.58 (dd, J = 9.6, 0.9 Hz, |
| | [1,2,4]triazolo[1,5- | | | | 1H), 7.05 (ddd, J = 8.1, 5.5, |
| | α]pyridin-6-yl)-4,5- | | | | 2.1 Hz, 1H), 6.88 (td, J = 9.4, |
| | dimethyl-5- | | | | 7.6 Hz, 1H), 5.01 (d, J = 10.5 |
| | (trifluoromethyl)tetrahydrofuran- | | | | Hz, 1H), 4.68 (s, 2H), 4.24 |
| | 2-carboxamide | | | | (dd, J = 10.5, 8.0 Hz, 1H), 3.90 |
| | | | | | (d, J = 2.2 Hz, 3H), 2.70 (p, J = |
| | | | | | 7.7 Hz, 1H), 1.58 (s, 3H), 0.72 |
| | | | | | (dq, J = 7.5, 2.3 Hz, 3H) ppm. |
| 443 | (2R,3S,4S,5R)-3-(3,4- | 500.419 | 501.3 | 3.11 | ¹H NMR (500 MHz, DMSO- |
| | difluoro-2- | | | | d₆) δ 10.73 (s, 1H), 8.81 (d, J = |
| | methoxyphenyl)-N-(2- | | | | 7.4 Hz, 1H), 8.14 (d, J = 2.2 |
| | (hydroxymethyl)- | | | | Hz, 1H), 7.30 (dd, J = 7.5, 2.2 |
| | [1,2,4]triazolo[1,5- | | | | Hz, 1H), 7.25-7.12 (m, 2H), |
| | α]pyridin-7-yl)-4,5- | | | | 5.13 (d, J = 10.2 Hz, 1H), 4.60 |
| | dimethyl-5- | | | | (s, 2H), 4.30 (dd, J = 10.2, 7.7 |
| | (trifluoromethyl)tetrahydrofuran- | | | | Hz, 1H), 3.97 (d, J = 2.0 Hz, |
| | 2-carboxamide | | | | 3H), 2.80 (p, J = 7.5 Hz, 1H), |
| | | | | | 1.62 (s, 3H), 0.81-0.68 (m, |
| | | | | | 3H) ppm. |

The following compound was made using a method similar to that described in Example 10, except that 5-benzyloxy-2-chloro-pyrimidine was used in step 2 with 2-methyl tetrahydrofuran as solvent. Benzyl deprotection using General Method R with wet Degussa Pd/C was carried out as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 444 | (2R,3S,4S,5R)-3- | 447.356 | 448.3 | 2.53 | ¹H NMR (500 MHz, DMSO-d₆) |
| | (3,4-difluoro-2- | | | | δ 10.43 (s, 1H), 8.13 (s, 2H), |
| | methoxyphenyl)-N- | | | | 7.26-7.08 (m, 2H), 5.14 (d, J = |
| | (5-hydroxypyrimidin-2- | | | | 10.5 Hz, 1H), 4.28-4.13 (m, |
| | yl)-4,5-dimethyl-5- | | | | 1H), 3.94 (d, J = 2.1 Hz, 3H), |
| | (trifluoromethyl)tetrahydrofuran- | | | | 2.81-2.65 (m, 1H), 2.54 (s, |
| | 2-carboxamide | | | | 1H), 1.58 (s, 3H), 0.70 (d, J = |
| | | | | | 7.4 Hz, 3H) ppm. |

The following compound was made using a method similar to that described in Example 10, except that tert-butyl N-[2-benzyloxy-1-(4-chloro-5-fluoro-2-pyridyl)ethyl]-N-methyl-carbamate was used in step 2. The diastereomeric products generated in step 2 were separated by chiral SFC using a Chiralpak IG column, 5 μm particle size, 25 cm×20 mm from Daicel and General Method I and then General Method R were used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 22 | rel- | 521.453 | 523.4 | 3.26 | ¹H NMR (500 MHz, DMSO- |
| | (2R*,3S*,4S*,5R*)-3- | | | | d₆) δ 10.46 (s, 1H), 8.64 (s, |
| | (3,4-difluoro-2- | | | | 1H), 8.28 (s, 1H), 7.19 (s, 2H), |
| | methoxyphenyl)-N-(5- | | | | 5.38 (d, J = 10.4 Hz, 1H), 5.35 |
| | fluoro-2-(2-hydroxy-1- | | | | (s, 1H), 4.31 (d, J = 5.7 Hz, |
| | (methylamino)ethyl)pyridin- | | | | 1H), 4.27 (dd, J = 10.4, 7.5 Hz, |
| | 4-yl)-4,5-dimethyl-5- | | | | 1H), 3.96 (s, 3H), 3.72 (dq, J = |
| | (trifluoromethyl)tetrahydrofuran- | | | | 28.6, 6.0, 5.5 Hz, 2H), 3.28 (s, |
| | 2-carboxamide | | | | 1H), 2.79 (p, J = 7.2 Hz, 1H), |
| | (precursor was first | | | | 2.44 (s, 3H), 1.61 (s, 3H), |
| | eluting isomer by SFC | | | | 0.74 (d, J = 7.4 Hz, 3H) ppm. |
| | using Chiralpak IG | | | | |
| | column) | | | | |

The following compounds were made using a method similar to that described in Example 10, except conditions similar to that described in General method Q were used in the amination step 2 with different coupling partners.

| Cmpd | | LC/MS | Found | MS | |
|---|---|---|---|---|---|
| No. | Compound Name | (m/z calc.) | M + 1 | r.t. | NMR (shifts in ppm) |
| 445 | (2R,3S,4S,5R)-3-(3,4- | 477.425 | 478 | 2.91 | ¹H NMR (400 MHz, |
| | difluoro-2- | | | | DMSO-d₆) δ 9.86 (s, 1H), |
| | methoxyphenyl)-N-(2- | | | | 7.24-7.15 (m, 2H), 6.68 (s, |
| | (2-hydroxyethyl)-1- | | | | 1H), 5.11 (d, J = 10.6 Hz, |
| | methyl-1H-imidazol-5- | | | | 1H), 4.73 (s, 1H), 4.18 (dd, |
| | yl)-4,5-dimethyl-5- | | | | J = 10.6, 7.6 Hz, 1H), 3.94 |
| | (trifluoromethyl)tetrahydrofuran- | | | | (d, J = 2.2 Hz, 3H), 3.66 |
| | 2-carboxamide | | | | (m, 2H), 3.20 (s, 3H), 2.74 |
| | | | | | (m, 3H), 1.61 (s, 3H), 0.74 |
| | | | | | (d, J = 7.4 Hz, 3H) ppm. |
| 21 | (2R,3S,4S,5R)-3-(3,4- | 463.398 | 464 | 2.91 | ¹H NMR (400 MHz, |
| | difluoro-2- | | | | DMSO-d₆) δ 9.90 (s, 1H), |
| | methoxyphenyl)-N-(2- | | | | 7.30-7.11 (m, 2H), 6.66 (s, |
| | (hydroxymethyl)-1- | | | | 1H), 5.21 (s, 1H), 5.10 (d, J = |
| | methyl-1H-imidazol-5- | | | | 10.6 Hz, 1H), 4.40 (s, |
| | yl)-4,5-dimethyl-5- | | | | 2H), 4.18 (dd, J = 10.5, 7.6 |
| | (trifluoromethyl)tetrahydrofuran- | | | | Hz, 1H), 3.94 (d, J = 2.1 |
| | 2-carboxamide | | | | Hz, 3H), 3.28 (s, 3H), 2.74 |
| | | | | | (p, J = 7.6 Hz, 1H), 1.61 (s, |
| | | | | | 3H), 0.74 (d, J = 7.0 Hz, |
| | | | | | 3H) ppm. |

The following compounds were made using conditions similar to that described in Example 10, except 5-bromo-2-vinylpyridine was used as the coupling partner in step 2. Reaction conditions similar to General Method T were applied to the product of step 2. A mixture of epimers was isolated from both peaks in the SFC purification (General Method T, step 2). Both mixtures were treated with TBAF in dioxane at 80° C. in step 3 to give 446 as a 1:1 mixture of epimers. In the Table below, “MS r.t.” stands for Mass Spec retention time.

| | | LC/MS | | | |
|---|---|---|---|---|---|
| Cmpd | | (m/z | Found | MS | |
| No. | Compound Name | calc.) | M + 1 | r.t. | NMR (shifts in ppm) |
| 446 | (2R,3S,4S,5R)-3-(3,4- | 493.4 | 494.6 | 3.17 | ¹H NMR (500 MHz, DMSO- |
| | difluoro-2- | | | | d₆) δ 10.59 (s, 1H), 9.05 (s, |
| | methoxyphenyl)-N-(2- | | | | 2H), 7.24-7.09 (m, 2H), 5.47 |
| | (1-fluoro-2- | | | | (ddd, J = 48.9, 6.8, 4.0 Hz, |
| | hydroxyethyl)pyrimidin- | | | | 1H), 5.21-5.03 (m, 2H), 4.24 |
| | 5-yl)-4,5-dimethyl-5- | | | | (dd, J = 10.2, 7.7 Hz, 1H), 3.94 |
| | (trifluoromethyl)tetrahydrofuran- | | | | (d, J = 2.0 Hz, 3H), 3.92-3.73 |
| | 2-carboxamide, as a | | | | (m, 2H), 2.77 (p, J = 7.4 Hz, |
| | mixture of epimers at | | | | 1H), 1.61 (s, 3H), 0.77-0.68 |
| | the 1-fluoro-2- | | | | (m, 3H). |
| | hydroxyethyl group | | | | |
| | (precursor was first | | | | |
| | eluting isomer by SFC | | | | |
| | using OJ column) | | | | |

The following compound was made using a method similar to that described in Example 10, except 5-bromo-1,2-dimethyl-1H-imidazole was used as the coupling partner in step 2 with CuI, Cs₂CO₃ and (1R,2R)-cyclohexane-1,2-diamine; (1S,2S)-cyclohexane-1,2-diamine in dioxane at 100° C. instead of the conditions described above. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 447 | (2R,3S,4S,5R)-3-(3,4- | 447.399 | 448 | 3.09 | ¹H NMR (400 MHz, DMSO-d₆) |
| | difluoro-2- | | | | δ 10.40 (s, 1H), 7.44 (s, 1H), |
| | methoxyphenyl)-N-(1,2- | | | | 7.20-7.06 (m, 2H), 5.15 (d, J = |
| | dimethyl-1H-imidazol-5- | | | | 10.4 Hz, 1H), 4.14 (dd, J = 10.5, |
| | yl)-4,5-dimethyl-5- | | | | 7.6 Hz, 1H), 3.88 (d, J = 2.1 Hz, |
| | (trifluoromethyl)tetrahydrofuran- | | | | 3H), 3.35 (s, 3H), 2.70 (p, J = |
| | 2-carboxamide | | | | 7.5 Hz, 1H), 2.47 (s, 3H), 1.55 |
| | (TFA salt) | | | | (s, 3H), 0.68 (dd, J = 7.5, 2.5 |
| | | | | | Hz, 3H) ppm. |

The following compounds were made from using a method similar to that described in Example 10 except that rac-(2R,3S,4S,5R)-3-(3,4-difluoro-2-methoxyphenyl)-4,5-dimethyl-5-(trifluoromethyl)tetrahydrofuran-2-carboxylic acid (product of step 10 in Example 7) was used in step 1 and different coupling partners were used in step 2. Products were separated by chiral SFC as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 448 | rel-(2S,3R,4R,5S)-3- | 537.5 | 538.1 | 3.52 | ¹H NMR (500 MHz, |
| | (3,4-difluoro-2- | | | | Methanol-d₄) δ 8.56 (d, J = 5.6 |
| | methoxyphenyl)-N-(2- | | | | Hz, 1H), 8.28 (d, J = 2.0 Hz, |
| | (N,N-dimethylsulfamoyl)pyridin- | | | | 1H), 7.90 (dd, J = 5.5, 2.1 Hz, |
| | 4-yl)-4,5-dimethyl-5- | | | | 1H), 7.14 (ddd, J = 8.1, 5.5, |
| | (trifluoromethyl)tetrahydrofuran- | | | | 2.1 Hz, 1H), 7.00 (ddd, J = 9.9, |
| | 2-carboxamide | | | | 8.9, 7.5 Hz, 1H), 5.11 (d, J = |
| | (first eluting isomer by | | | | 10.4 Hz, 1H), 4.35 (dd, J = |
| | SFC using AS-H | | | | 10.4, 8.0 Hz, 1H), 4.02 (d, J = |
| | column) | | | | 2.3 Hz, 3H), 2.87 (s, 6H), 2.82 |
| | | | | | (p, J = 7.7 Hz, 1H), 1.68 (d, J = |
| | | | | | 1.3 Hz, 3H), 0.83 (dq, J = 7.4, |
| | | | | | 2.3 Hz, 3H) ppm. |
| 449 | rel-(2R,3S,4S,5R)-3- | 537.5 | 538.1 | 3.52 | ¹H NMR (500 MHz, |
| | (3,4-difluoro-2- | | | | Methanol-d₄) δ 8.45 (dd, J = |
| | methoxyphenyl)-N-(2- | | | | 5.5, 0.6 Hz, 1H), 8.16 (dd, J = |
| | (N,N-dimethylsulfamoyl)pyridin- | | | | 2.2, 0.6 Hz, 1H), 7.78 (dd, J = |
| | 4-yl)-4,5-dimethyl-5- | | | | 5.5, 2.1 Hz, 1H), 7.01 (ddt, J = |
| | (trifluoromethyl)tetrahydrofuran- | | | | 10.8, 7.4, 3.7 Hz, 1H), 6.94- |
| | 2-carboxamide | | | | 6.83 (m, 1H), 4.99 (d, J = 10.4 |
| | (second eluting isomer | | | | Hz, 1H), 4.22 (td, J = 10.4, 7.8 |
| | by SFC using AS-H | | | | Hz, 1H), 3.90 (d, J = 2.3 Hz, |
| | column) | | | | 3H), 2.75 (s, 6H), 2.69 (q, J = |
| | | | | | 7.7 Hz, 1H), 1.56 (d, J = 1.1 |
| | | | | | Hz, 3H), 0.70 (ddq, J = 14.1, |
| | | | | | 7.2, 2.3 Hz, 4H) ppm. |
| 450 | rel-(2S,3R,4R,5S)-3- | 471.381 | 472.6 | 3.32 | ¹H NMR (500 MHz, DMSO- |
| | (3,4-difluoro-2- | | | | d₆) δ 10.73 (s, 1H), 9.71 (dd, J = |
| | methoxyphenyl)-4,5- | | | | 1.9, 0.9 Hz, 1H), 8.21 (dd, J = |
| | dimethyl-N- | | | | 9.6, 0.9 Hz, 1H), 7.90 (dd, J = |
| | (tetrazolo[1,5- | | | | 9.6, 1.8 Hz, 1H), 7.19-7.17 |
| | α]pyridin-6-yl)-5- | | | | (m, 2H), 5.17 (d, J = 10.2 Hz, |
| | (trifluoromethyl)tetrahydrofuran- | | | | 1H), 4.30 (dd, J = 10.2, 7.7 Hz, |
| | 2-carboxamide | | | | 1H), 3.97 (d, J = 2.1 Hz, 3H), |
| | (first eluting isomer by | | | | 2.80 (p, J = 7.5 Hz, 1H), 1.63 |
| | SFC using Whelk-O1 | | | | (s, 3H), 0.76-0.74 (m, 3H) |
| | column) | | | | ppm. |
| 451 | rel-(2R,3S,4S,5R)-3- | 471.381 | 472.6 | 3.31 | ¹H NMR (500 MHz, DMSO- |
| | (3,4-difluoro-2- | | | | d₆) δ 10.75 (s, 1H), 9.71-9.70 |
| | methoxyphenyl)-4,5- | | | | (m, 1H), 8.21 (dd, J = 9.6, 0.9 |
| | dimethyl-N- | | | | Hz, 1H), 7.89 (dd, J = 9.6, 1.8 |
| | (tetrazolo[1,5- | | | | Hz, 1H), 7.19-7.17 (m, 2H), |
| | α]pyridin-6-yl)-5- | | | | 5.17 (d, J = 10.2 Hz, 1H), 4.30 |
| | (trifluoromethyl)tetrahydrofuran- | | | | (dd, J = 10.2, 7.7 Hz, 1H), 3.97 |
| | 2-carboxamide | | | | (d, J = 2.0 Hz, 3H), 2.79 (p, J = |
| | (second eluting isomer | | | | 7.5 Hz, 1H), 1.63 (s, 3H), 0.76- |
| | by SFC using Whelk- | | | | 0.74 (m, 3H) ppm. |
| | O1 column) | | | | |
| 452 | rel-(2S,3R,4R,5S)-3- | 537.5 | 538.1 | 3.45 | ¹H NMR (500 MHz, |
| | (3,4-difluoro-2- | | | | Chloroform-d) δ 8.93 (d, J = |
| | methoxyphenyl)-N-(5- | | | | 2.4 Hz, 1H), 8.75 (d, J = 1.9 |
| | (N,N-dimethylsulfamoyl)pyridin- | | | | Hz, 1H), 8.62 (s, 1H), 8.46 (t, J = |
| | 3-yl)-4,5-dimethyl-5- | | | | 2.1 Hz, 1H), 7.09 (ddd, J = |
| | (trifluoromethyl)tetrahydrofuran- | | | | 8.0, 5.5, 2.1 Hz, 1H), 6.92 (td, |
| | 2-carboxamide | | | | J = 9.2, 7.4 Hz, 1H), 5.06 (d, J = |
| | (first eluting isomer by | | | | 10.9 Hz, 1H), 4.14 (dd, J = |
| | SFC using AS-H | | | | 11.0, 8.1 Hz, 1H), 4.04 (d, J = |
| | column) | | | | 2.8 Hz, 3H), 2.92-2.66 (m, |
| | | | | | 7H), 1.72 (s, 3H), 0.81 (dq, J = |
| | | | | | 7.4, 2.3 Hz, 3H) ppm. |
| 453 | rel-(2R,3S,4S,5R)-3- | 537.5 | 538.1 | 3.45 | ¹H NMR (500 MHz, |
| | (3,4-difluoro-2- | | | | Chloroform-d) δ 8.92 (d, J = |
| | methoxyphenyl)-N-(5- | | | | 2.5 Hz, 1H), 8.76 (d, J = 1.9 |
| | (N,N-dimethylsulfamoyl)pyridin- | | | | Hz, 1H), 8.59 (s, 1H), 8.45 (t, J = |
| | 3-yl)-4,5-dimethyl-5- | | | | 2.2 Hz, 1H), 7.09 (ddd, J = |
| | (trifluoromethyl)tetrahydrofuran- | | | | 8.0, 5.5, 2.1 Hz, 1H), 6.93 (td, |
| | 2-carboxamide | | | | J = 9.3, 7.4 Hz, 1H), 5.06 (d, J = |
| | (second eluting isomer | | | | 11.0 Hz, 1H), 4.14 (dd, J = |
| | by SFC using AS-H | | | | 11.0, 8.1 Hz, 1H), 4.04 (d, J = |
| | column) | | | | 2.8 Hz, 3H), 2.79 (s, 7H), 1.72 |
| | | | | | (d, J = 1.2 Hz, 3H), 0.82 (dq, J = |
| | | | | | 7.4, 2.3 Hz, 3H) ppm. |

The following compound was made using a method similar to that described in Example 10 except rac-(2R,3S,4S,5R)-3-(3,4-difluoro-2-methoxyphenyl)-4,5-dimethyl-5-(trifluoromethyl)tetrahydrofuran-2-carboxylic acid (product of step 10 in Example 7) was used in step 1 and 6-bromotetrazolo[1,5-a]pyridine was used as coupling partner in step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 454 | rac-(2R,3S,4S,5R)-3- | 471.381 | 472.6 | 3.30 | ¹H NMR (500 MHz, DMSO-d₆) |
| | (3,4-difluoro-2- | | | | δ 10.80 (s, 1H), 9.71 (dd, J = |
| | methoxyphenyl)-4,5- | | | | 1.8, 0.9 Hz, 1H), 8.21 (d, J = 9.5 |
| | dimethyl-N- | | | | Hz, 1H), 7.90-7.88 (m, 1H), |
| | (tetrazolo[1,5- | | | | 7.19-7.17 (m, 2H), 5.17 (d, J = |
| | α]pyridin-6-yl)-5- | | | | 10.1 Hz, 1H), 4.30 (dd, J = 10.2, |
| | (trifluoromethyl)tetrahydrofuran- | | | | 7.7 Hz, 1H), 3.97 (d, J = 2.1 Hz, |
| | 2-carboxamide | | | | 3H), 2.83-2.77 (m, 1H), 1.63 |
| | | | | | (s, 3H), 0.76-0.75 (m, 3H) |
| | | | | | ppm. |

The following compounds were made using a method similar to that described in Example 10, except that rac-(2R,3S,4S,5R)-3-(3,4-difluoro-2-methoxyphenyl)-4,5-dimethyl-5-(trifluoromethyl)tetrahydrofuran-2-carboxylic acid (from Example 7, step 10) was used in step 1 and 4-bromo-N-methyl-N-(2-trimethylsilylethoxymethyl)pyridine-2-sulfonamide was used as coupling partner in step 2. MOM deprotection was done using conditions similar to General Method M at 60° C. and chiral SFC using a Chiralpak AS-H column, 5 μm particle size, 25 cm×10 mm from Daicel on a Minigram SFC instrument from Berger Instruments was used to separate the enantiomers in the final step. In the

| Cmpd | | LC/MS | Found | MS | NMR |
|---|---|---|---|---|---|
| No. | Compound Name | (m/z calc.) | M + 1 | r.t. | (shifts in ppm) |
| 455 | rel-(2S,3R,4R,5S)-3- | 523.473 | 524.5 | 3.38 | |
| | (3,4-difluoro-2- | | | | |
| | methoxyphenyl)-4,5- | | | | |
| | dimethyl-N-(2-(N- | | | | |
| | methylsulfamoyl)pyridin- | | | | |
| | 4-yl)-5- | | | | |
| | (trifluoromethyl)tetrahydrofuran- | | | | |
| | 2-carboxamide | | | | |
| | (first eluting isomer by | | | | |
| | SFC) | | | | |
| 456 | rel-(2R,3S,4S,5R)-3- | 523.473 | 524.5 | 3.37 | |
| | (3,4-difluoro-2- | | | | |
| | methoxyphenyl)-4,5- | | | | |
| | dimethyl-N-(2-(N- | | | | |
| | methylsulfamoyl)pyridin- | | | | |
| | 4-yl)-5- | | | | |
| | (trifluoromethyl)tetrahydrofuran- | | | | |
| | 2-carboxamide | | | | |
| | (second eluting isomer | | | | |
| | by SFC) | | | | |

The following compounds were made using conditions similar to that described in Example 10 except that rac-(2R,3S,4S,5R)-3-(2-(difluoromethoxy)-3,4-difluorophenyl)-4,5-dimethyl-5-(trifluoromethyl)tetrahydrofuran-2-carboxylic acid (from Example 2, step 1) was used in step 1 and different coupling partners were used in step 2. Enantiomers were separated by chiral SFC in the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 457 | rel-(2S,3R,4R,5S)-3- | 510.402 | 511.6 | 3.19 | ¹H NMR (500 MHz, DMSO- |
| | (2-(difluoromethoxy)- | | | | d₆) δ 10.47 (s, 1H), 8.34 (d, J = |
| | 3,4-difluorophenyl)-N- | | | | 5.5 Hz, 1H), 7.50 (d, J = 2.0 |
| | (2-(2- | | | | Hz, 1H), 7.48 (d, J = 8.3 Hz, |
| | hydroxyethyl)pyridin- | | | | 1H), 7.43 (dd, J = 5.6, 2.0 Hz, |
| | 4-yl)-4,5-dimethyl-5- | | | | 1H), 7.32-7.29 (m, 1H), 7.43- |
| | (trifluoromethyl)tetrahydrofuran- | | | | 7.13 (m, 1H), 5.13 (d, J = |
| | 2-carboxamide | | | | 10.3 Hz, 1H), 4.61 (t, J = 5.3 |
| | (first eluting isomer by | | | | Hz, 1H), 4.28 (dd, J = 10.3, 7.6 |
| | SFC using Lux | | | | Hz, 1H), 3.71 (td, J = 6.6, 5.0 |
| | Cellulose-2 column) | | | | Hz, 2H), 2.80 (t, J = 6.7 Hz, |
| | | | | | 2H), 2.76 (t, J = 7.5 Hz, 1H), |
| | | | | | 1.59 (s, 3H), 0.76 (d, J = 7.4 |
| | | | | | Hz, 3H) ppm. |
| 458 | rel-(2R,3S,4S,5R)-3- | 510.402 | 511.6 | 3.19 | ¹H NMR (500 MHz, DMSO- |
| | (2-(difluoromethoxy)- | | | | d₆) δ 10.46 (s, 1H), 8.34 (d, J = |
| | 3,4-difluorophenyl)-N- | | | | 5.4 Hz, 1H), 7.50 (d, J = 2.0 |
| | (2-(2- | | | | Hz, 1H), 7.48 (d, J = 8.4 Hz, |
| | hydroxyethyl)pyridin- | | | | 1H), 7.43 (dd, J = 5.6, 2.0 Hz, |
| | 4-yl)-4,5-dimethyl-5- | | | | 1H), 7.31-7.29 (m, 1H), 7.43- |
| | (trifluoromethyl)tetrahydrofuran- | | | | 7.14 (m, 1H), 5.13 (d, J = |
| | 2-carboxamide | | | | 10.2 Hz, 1H), 4.61 (t, J = 5.3 |
| | (second eluting isomer | | | | Hz, 1H), 4.28 (dd, J = 10.2, 7.6 |
| | by SFC using Lux | | | | Hz, 1H), 3.71 (td, J = 6.7, 5.2 |
| | Cellulose-2 column) | | | | Hz, 2H), 2.81 (t, J = 6.7 Hz, |
| | | | | | 2H), 2.76 (t, J = 7.5 Hz, 1H), |
| | | | | | 1.59 (s, 3H), 0.76 (d, J = 7.3 |
| | | | | | Hz, 3H) ppm. |
| 459 | rel-(2S,3R,4R,5S)-3- | 510.402 | 511.1 | 3.12 | ¹H NMR (500 MHz, DMSO- |
| | (2-(difluoromethoxy)- | | | | d₆) δ 10.29 (s, 1H), 8.63 (d, J = |
| | 3,4-difluorophenyl)-N- | | | | 2.5 Hz, 1H), 7.90 (dd, J = 8.4, |
| | (6-(2- | | | | 2.6 Hz, 1H), 7.47 (td, J = 9.5, |
| | hydroxyethyl)pyridin- | | | | 7.7 Hz, 1H), 7.33 (ddd, J = 8.8, |
| | 3-yl)-4,5-dimethyl-5- | | | | 5.6, 1.9 Hz, 1H), 7.28 (t, J = |
| | (trifluoromethyl)tetrahydrofuran- | | | | 72.4 Hz, 1H), 7.22 (d, J = 8.4 |
| | 2-carboxamide | | | | Hz, 1H), 5.13 (d, J = 10.3 Hz, |
| | (first eluting isomer by | | | | 1H), 4.59 (t, J = 5.3 Hz, 1H), |
| | SFC using Lux | | | | 4.26 (dd, J = 10.3, 7.6 Hz, 1H), |
| | Cellulose-2 column) | | | | 3.69 (td, J = 6.8, 5.1 Hz, 2H), |
| | | | | | 2.82 (t, J = 6.8 Hz, 2H), 2.75 |
| | | | | | (p, J = 7.6 Hz, 1H), 1.60 (s, |
| | | | | | 3H), 0.76 (d, J = 6.8 Hz, 3H) |
| | | | | | ppm. |
| 460 | rel-(2R,3S,4S,5R)-3- | 510.402 | 511.1 | 3.13 | ¹H NMR (500 MHz, DMSO- |
| | (2-(difluoromethoxy)- | | | | d₆) δ 10.30 (s, 1H), 8.63 (d, J = |
| | 3,4-difluorophenyl)-N- | | | | 2.5 Hz, 1H), 7.90 (dd, J = 8.4, |
| | (6-(2- | | | | 2.6 Hz, 1H), 7.50-7.42 (m, |
| | hydroxyethyl)pyridin- | | | | 1H), 7.35-7.31 (m, 1H), 7.28 |
| | 3-yl)-4,5-dimethyl-5- | | | | (t, J = 72.2 Hz, 1H), 7.22 (d, |
| | (trifluoromethyl)tetrahydrofuran- | | | | J = 8.4 Hz, 1H), 5.13 (d, J = |
| | 2-carboxamide | | | | 10.3 Hz, 1H), 4.59 (t, J = 5.3 |
| | (second eluting isomer | | | | Hz, 1H), 4.26 (dd, J = 10.3, 7.6 |
| | by SFC using Lux | | | | Hz, 1H), 3.69 (td, J = 6.7, 4.8 |
| | Cellulose-2 column) | | | | Hz, 2H), 2.82 (t, J = 6.8 Hz, |
| | | | | | 2H), 2.75 (p, J = 7.5 Hz, 1H), |
| | | | | | 1.60 (s, 3H), 0.76 (d, J = 5.1 |
| | | | | | Hz, 3H) ppm. |
| 461 | rel-(2S,3R,4R,5S)-3- | 538.455 | 539.6 | 3.35 | ¹H NMR (500 MHz, DMSO- |
| | (2-(difluoromethoxy)- | | | | d₆) δ 10.30 (s, 1H), 8.63 (d, J = |
| | 3,4-difluorophenyl)-N- | | | | 2.5 Hz, 1H), 7.91 (dd, J = 8.5, |
| | (6-(2-hydroxy-2- | | | | 2.6 Hz, 1H), 7.48 (td, J = 9.5, |
| | methylpropyl)pyridin- | | | | 7.7 Hz, 1H), 7.34-7.30 (m, |
| | 3-yl)-4,5-dimethyl-5- | | | | 1H), 7.28 (t, J = 72.2 Hz, 1H), |
| | (trifluoromethyl)tetrahydrofuran- | | | | 7.25 (d, J = 8.5 Hz, 1H), 5.13 |
| | 2-carboxamide | | | | (d, J = 10.3 Hz, 1H), 4.59 (s, |
| | (first eluting isomer by | | | | 1H), 4.27 (dd, J = 10.3, 7.6 Hz, |
| | SFC using Lux | | | | 1H), 2.78 (s, 2H), 2.77-2.73 |
| | Cellulose-2 column) | | | | (m, 1H), 1.60 (s, 3H), 1.06 (s, |
| | | | | | 6H), 0.76 (d, J = 6.7 Hz, 3H) |
| | | | | | ppm. |
| 462 | rel-(2R,3S,4S,5R)-3- | 538.455 | 539.6 | 3.35 | ¹H NMR (500 MHz, DMSO- |
| | (2-(difluoromethoxy)- | | | | d₆) δ 10.30 (s, 1H), 8.63 (s, |
| | 3,4-difluorophenyl)-N- | | | | 1H), 7.91 (d, J = 6.5 Hz, 1H), |
| | (6-(2-hydroxy-2- | | | | 7.48 (q, J = 8.9 Hz, 1H), 7.34- |
| | methylpropyl)pyridin- | | | | 7.31 (m, 1H), 7.28 (t, J = 72.3 |
| | 3-yl)-4,5-dimethyl-5- | | | | Hz, 1H), 7.25 (d, J = 8.5 Hz, |
| | (trifluoromethyl)tetrahydrofuran- | | | | 1H), 5.13 (d, J = 10.3 Hz, 1H), |
| | 2-carboxamide | | | | 4.59 (s, 1H), 4.27 (t, J = 9.0 |
| | (second eluting isomer | | | | Hz, 1H), 2.78 (s, 2H), 2.77- |
| | by SFC using Lux | | | | 2.73 (m, 1H), 1.60 (s, 3H), |
| | Cellulose-2 column) | | | | 1.06 (s, 6H), 0.76 (d, J = 7.3 |
| | | | | | Hz, 3H) ppm. |
| 463 | rel-(2S,3R,4R,5S)-3- | 538.455 | 539.7 | 3.39 | ¹H NMR (500 MHz, DMSO- |
| | (2-(difluoromethoxy)- | | | | d₆) δ 10.49 (s, 1H), 8.34 (d, J = |
| | 3,4-difluorophenyl)-N- | | | | 5.6 Hz, 1H), 7.53 (d, J = 2.1 |
| | (2-(2-hydroxy-2- | | | | Hz, 1H), 7.50-7.45 (m, 2H), |
| | methylpropyl)pyridin- | | | | 7.31-7.28 (m, 1H), 7.29 (t, J = |
| | 4-yl)-4,5-dimethyl-5- | | | | 72.1 Hz, 1H), 5.14 (d, J = |
| | (trifluoromethyl)tetrahydrofuran- | | | | 10.2 Hz, 1H), 4.78 (s, 1H), |
| | 2-carboxamide | | | | 4.29 (dd, J = 10.3, 7.7 Hz, 1H), |
| | (first eluting isomer by | | | | 2.79-2.73 (m, 1H), 2.76 (s, |
| | SFC using Lux | | | | 2H), 1.59 (s, 3H), 1.08 (d, J = |
| | Cellulose-2 column) | | | | 2.8 Hz, 6H), 0.76 (d, J = 7.3 |
| | | | | | Hz, 3H) ppm. |
| 464 | rel-(2R,3S,4S,5R)-3- | 538.455 | 539.6 | 3.4 | ¹H NMR (500 MHz, DMSO- |
| | (2-(difluoromethoxy)- | | | | d₆) δ 10.50 (s, 1H), 8.34 (d, J = |
| | 3,4-difluorophenyl)-N- | | | | 5.5 Hz, 1H), 7.53 (d, J = 1.4 |
| | (2-(2-hydroxy-2- | | | | Hz, 1H), 7.50-7.46 (m, 2H), |
| | methylpropyl)pyridin- | | | | 7.31-7.28 (m, 1H), 7.29 (t, |
| | 4-yl)-4,5-dimethyl-5- | | | | J = 72.1 Hz, 1H), 5.14 (d, J = |
| | (trifluoromethyl)tetrahydrofuran- | | | | 10.2 Hz, 1H), 4.78 (s, 1H), |
| | 2-carboxamide | | | | 4.29 (dd, J = 10.2, 7.7 Hz, 1H), |
| | (second eluting isomer | | | | 2.79-2.72 (m, 1H), 2.76 (s, |
| | by SFC using Lux | | | | 2H), 1.59 (s, 3H), 1.08 (d, J = |
| | Cellulose-2 column) | | | | 2.8 Hz, 6H), 0.76 (d, J = 7.4 |
| | | | | | Hz, 3H) ppm. |

### Example 11

(2R,3S,4S,5R)-3-(3,4-difluoro-2-methoxyphenyl)-N-(2-((R)-2,4-dimethyl-6-oxopiperazin-1-yl)pyridin-4-yl)-4,5-dimethyl-5-(trifluoromethyl)tetrahydrofuran-2-carboxamide (465)

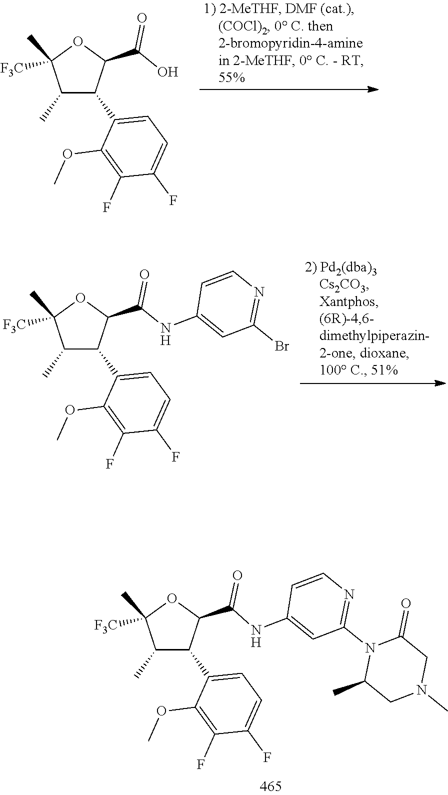 [SMILES: COc1c([C@H]2[C@H](C(=O)O)O[C@@](C)(C(F)(F)F)[C@H]2C)ccc(F)c1F ; COc1c([C@H]2[C@H](C(=O)Nc3ccnc(Br)c3)O[C@@](C)(C(F)(F)F)[C@H]2C)ccc(F)c1F ; COc1c([C@H]2[C@H](C(=O)Nc3ccnc(N4C(=O)CN(C)C[C@H]4C)c3)O[C@@](C)(C(F)(F)F)[C@H]2C)ccc(F)c1F]

### Step 1:

To an ice cooled solution of (2R,3S,4S,5R)-3-(3,4-difluoro-2-methoxyphenyl)-4,5-dimethyl-5-(trifluoromethyl)tetrahydrofuran-2-carboxylic acid (0.59 g, 1.6188 mmol) in 2-methyltetrahydrofuran (6.5 mL) was added DMF (11.328 mg, 12 μL, 0.1550 mmol) and oxalyl chloride (320.10 mg, 220 μL, 2.5219 mmol). The reaction mixture was warmed to room temperature and stirred for 2 hrs. The reaction mixture was concentrated in vacuo and the residue dissolved in 2-methyltetrahydrofuran (6.5 mL). This solution was added to an ice-cooled solution of 2-bromopyridin-4-amine (256 mg, 1.4797 mmol) and triethylamine (246.84 mg, 0.34 mL, 2.4394 mmol) in 2-methyltetrahydrofuran (6.5 mL). The resulting mixture was warmed to ambient temperature and stirred for 1.5 hours. The reaction mixture was quenched with water (20 mL) and partitioned with ethyl acetate (40 mL). The layers were separated and the organic was washed with brine (20 mL), dried (sodium sulfate), filtered and concentrated under reduced pressure to obtain a yellow solid (809 mg). The residue was purified by flash column chromatography (40 g SiO₂, 0 to 100% EtOAc in heptanes) to give (2R,3S,4S,5R)—N-(2-bromopyridin-4-yl)-3-(3,4-difluoro-2-methoxyphenyl)-4,5-dimethyl-5-(trifluoromethyl)tetrahydrofuran-2-carboxamide (471 mg, 55%) as a white solid. ¹H-NMR (400 MHz, DMSO-d₆) δ 10.65 (s, 1H), 8.22 (d, J=5.7 Hz, 1H), 7.90 (d, J=1.6 Hz, 1H), 7.56 (dd, J=5.6, 1.9 Hz, 1H), 7.16-7.05 (m, 2H), 5.05 (d, J=10.1 Hz, 1H), 4.20 (dd, J=10.1, 7.8 Hz, 1H), 3.90 (d, J=2.1 Hz, 3H), 2.72 (m, 1H), 1.56 (s, 3H), 0.68 (d, J=5.7 Hz, 3H) ppm. ESI-MS m/z calc. 508.0421, found 511.01 (M+1)⁺; Retention time: 1.14 minutes.

### Step 2:

(2R,3S,4S,5R)—N-(2-bromopyridin-4-yl)-3-(3,4-difluoro-2-methoxyphenyl)-4,5-dimethyl-5-(trifluoromethyl)tetrahydrofuran-2-carboxamide (120 mg, 0.2356 mmol), (6R)-4,6-dimethylpiperazin-2-one (45 mg, 0.3335 mmol), cesium carbonate (150 mg, 0.4604 mmol), Pd₂(dba)₃ (10.5 mg, 0.0115 mmol) and Xantphos (14.8 mg, 0.0256 mmol) were suspended in 1,4-dioxane (2.40 mL) and heated to 100° C. for 18 hrs. The reaction was partitioned between DCM (10 mL) and washed with water (7 mL), the aqueous was washed with DCM (3×15 mL), the organic layers were combined, dried (sodium sulfate), filtered and concentrated under reduced pressure to obtain a pale orange oil. The crude was purified by flash column chromatography (25 g SiO₂, 0 to 10% MeOH in ethylacetate) and then freeze dried to give (2R,3S,4S,5R)-3-(3,4-difluoro-2-methoxyphenyl)-N-(2-((R)-2,4-dimethyl-6-oxopiperazin-1-yl)pyridin-4-yl)-4,5-dimethyl-5-(trifluoromethyl)tetrahydrofuran-2-carboxamide (465, 69 mg, 51%) as a pale yellow solid. ¹H-NMR (400 MHz, Chloroform-d) δ 8.53 (s, 1H), 8.33 (d, J=5.5 Hz, 1H), 7.64 (d, J=1.6 Hz, 1H), 7.61 (dd, J=5.6, 1.9 Hz, 1H), 7.07-7.03 (m, 1H), 6.87 (td, J=9.2, 7.5 Hz, 1H), 4.97 (d, J=11.0 Hz, 1H), 4.77-4.70 (m, 1H), 4.07 (dd, J=10.9, 8.1 Hz, 1H), 3.99 (d, J=2.7 Hz, 3H), 3.43 (dd, J=16.9, 1.1 Hz, 1H), 3.02 (d, J=16.7 Hz, 1H), 2.75-2.68 (m, 2H), 2.65 (ddd, J=11.8, 4.0, 1.3 Hz, 1H), 2.35 (s, 3H), 1.64 (s, 3H), 1.16 (d, J=6.4 Hz, 3H), 0.78-0.75 (m, 3H) ppm. ESI-MS m/z calc. 556.2109, found 557.18 (M+1)⁺; Retention time: 2.53 minutes.

The following compounds were made using a method similar to that described in Example 11, except that different amines were used in the Buchwald coupling step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 466 | (2R,3S,4S,5R)-3-(3,4- | 556.525 | 556.9 | 3.19 | ¹H NMR (500 MHz, |
| | difluoro-2- | | | | DMSO-d₆) δ 10.64 (s, 1H), |
| | methoxyphenyl)-4,5- | | | | 8.27 (d, J = 5.6 Hz, 1H), |
| | dimethyl-N-(2-(4-methyl- | | | | 7.93 (d, J = 1.9 Hz, 1H), |
| | 7-oxo-1,4-diazepan-1- | | | | 7.48 (dd, J = 5.6, 1.9 Hz, |
| | yl)pyridin-4-yl)-5- | | | | 1H), 7.22-7.06 (m, 2H), |
| | (trifluoromethyl)tetrahydrofuran- | | | | 5.08 (d, J = 10.3 Hz, 1H), |
| | 2-carboxamide | | | | 4.24 (dd, J = 10.3, 7.6 Hz, |
| | | | | | 1H), 4.14-4.02 (m, 2H), |
| | | | | | 3.95 (d, J = 2.0 Hz, 3H), |
| | | | | | 2.84-2.71 (m, 3H), 2.63- |
| | | | | | 2.52 (m, 4H), 2.24 (s, 3H), |
| | | | | | 1.59 (s, 3H), 0.77-0.64 (m, |
| | | | | | 3H) ppm. |
| 467 | (2R,3S,4S,5R)-3-(3,4- | 556.525 | 557.16 | 2.49 | ¹H-NMR (301 MHz, |
| | difluoro-2- | | | | Chloroform-d) δ 8.47 (s, |
| | methoxyphenyl)-4,5- | | | | 1H), 8.29 (d, J = 5.7 Hz, |
| | dimethyl-N-(2-(4-methyl- | | | | 1H), 7.69 (dd, J = 5.7, 1.9 |
| | 2-oxo-1,4-diazepan-1- | | | | Hz, 1H), 7.60 (d, J = 1.7 |
| | yl)pyridin-4-yl)-5- | | | | Hz, 1H), 7.10-7.05 (m, 1H), |
| | (trifluoromethyl)tetrahydrofuran- | | | | 6.93-6.85 (m,1H), 4.98 (d, |
| | 2-carboxamide | | | | J = 11.0 Hz, 1H), 4.17 (br. s, |
| | | | | | 1H), 4.05 (dd, J = 10.8, 8.4 |
| | | | | | Hz, 1H), 4.00 (d, J = 2.9 |
| | | | | | Hz, 3H), 3.69 (s, 2H), 3.01 |
| | | | | | (br. s, 2H), 2.78-2.68 (m, |
| | | | | | 1H), 2.55 (s, 3H), 2.01 (br. |
| | | | | | s, 3H), 1.66 (s, 3H), 0.78- |
| | | | | | 0.75 (m, 3H) ppm. |
| 468 | (2R,3S,4S,5R)-3-(3,4- | 568.536 | 568.9 | 3.15 | |
| | difluoro-2- | | | | |
| | methoxyphenyl)-4,5- | | | | |
| | dimethyl-N-(2-(3-oxo- | | | | |
| | 1,4- | | | | |
| | diazabicyclo[3.2.2]nonan- | | | | |
| | 4-yl)pyridin-4-yl)-5- | | | | |
| | (trifluoromethyl)tetrahydrofuran- | | | | |
| | 2-carboxamide | | | | |
| 469 | (2R,3S,4S,5R)-3-(3,4- | 556.525 | 557.2 | 2.54 | ¹H-NMR (400 MHz, |
| | difluoro-2- | | | | Chloroform-d) δ 8.44 (s, |
| | methoxyphenyl)-N-(2- | | | | 1H), 8.34 (d, J = 5.5 Hz, |
| | ((S)-2,4-dimethyl-6- | | | | 1H), 7.75 (dd, J = 5.6, 1.9 |
| | oxopiperazin-1- | | | | Hz, 1H), 7.51 (d, J = 1.8 |
| | yl)pyridin-4-yl)-4,5- | | | | Hz, 1H), 7.09-7.05 (m, 1H), |
| | dimethyl-5- | | | | 6.92-6.85 (m, 1H), 4.97 (d, |
| | (trifluoromethyl)tetrahydrofuran- | | | | J = 11.2 Hz, 1H), 4.78-4.72 |
| | 2-carboxamide | | | | (m, 1H), 4.05 (dd, J = 11.0, |
| | | | | | 8.2 Hz, 1H), 4.00 (d, J = 2.7 |
| | | | | | Hz, 3H), 3.44 (dd, J = 16.8, |
| | | | | | 1.0 Hz, 1H), 3.02 (d, J = |
| | | | | | 16.9 Hz, 1H), 2.77-2.63 (m, |
| | | | | | 3H), 2.35 (s, 3H), 1.66 (s, |
| | | | | | 3H), 1.15 (d, J = 6.4 Hz, |
| | | | | | 3H), 0.78-0.75 (m, 3H) |
| 470 | (2R,3S,4S,5R)-3-(3,4- | 556.525 | 557.19 | 2.56 | ¹H-NMR (400 MHz, |
| | difluoro-2- | | | | Chloroform-d) δ 8.44 (s, |
| | methoxyphenyl)-N-(2- | | | | 1H), 8.30 (d, J = 5.7 Hz, |
| | ((R)-4,5-dimethyl-2- | | | | 1H), 7.89 (d, J = 1.6 Hz, |
| | oxopiperazin-1- | | | | 1H), 7.71 (dd, J = 5.6, 1.9 |
| | yl)pyridin-4-yl)-4,5- | | | | Hz, 1H), 7.09-7.05 (m, 1H), |
| | dimethyl-5- | | | | 6.92-6.86 (m, 1H), 4.98 (d, |
| | (trifluoromethyl)tetrahydrofuran- | | | | J = 11.0 Hz, 1H), 4.10-4.04 |
| | 2-carboxamide | | | | (m, 2H), 4.00 (d, J = 3.0 |
| | | | | | Hz, 3H), 3.64-3.54 (m, 2H), |
| | | | | | 3.13 (d, J = 17.2 Hz, 1H), |
| | | | | | 2.73 (m, 1H), 2.66-2.61 (m, |
| | | | | | 1H), 2.32 (s, 3H), 1.66 (s, |
| | | | | | 3H), 1.21 (d, J = 6.2 Hz, |
| | | | | | 3H), 0.79-0.76 (m, 3H) |
| | | | | | ppm. |
| 471 | (2R,3S,4S,5R)-3-(3,4- | 529.456 | 530.13 | 2.45 | ¹H-NMR (400 MHz, |
| | difluoro-2- | | | | Chloroform-d) δ 8.51 (d, |
| | methoxyphenyl)-N-(2- | | | | J = 8.0 Hz, 1H), 8.25 (d, J = |
| | ((R)-3-hydroxy-2- | | | | 6.0 Hz, 1H), 8.23 (d, J = 2.3 |
| | oxopyrrolidin-1- | | | | Hz, 1H), 7.75-7.71 (m, 1H), |
| | yl)pyridin-4-yl)-4,5- | | | | 7.09-7.05 (m, 1H), 6.92- |
| | dimethyl-5- | | | | 6.86 (m, 1H), 5.00 (d, J = |
| | (trifluoromethyl)tetrahydrofuran- | | | | 10.8 Hz, 1H), 4.54-4.49 (m, |
| | 2-carboxamide | | | | 1H), 4.23 (dd, J = 11.0, 9.4 |
| | | | | | Hz, 1H), 4.12-4.06 (m, 1H), |
| | | | | | 4.00 (d, J = 2.7 Hz, 3H), |
| | | | | | 3.84-3.77 (m, 1H), 2.98 (s, |
| | | | | | 1H), 2.78-2.70 (m, 1H), |
| | | | | | 2.63-2.56 (m, 1H), 2.10- |
| | | | | | 1.99 (m, 1H), 1.68 (s, 3H), |
| | | | | | 0.79-0.77 (m, 3H) ppm. |
| 472 | (2R,3S,4S,5R)-3-(3,4- | 530.488 | 531.17 | 2.75 | ¹H-NMR (310 MHz, |
| | difluoro-2- | | | | Chloroform-d) δ 8.52 (s, |
| | methoxyphenyl)-N-(2-(2- | | | | 1H), 8.17 (d, J = 5.5 Hz, |
| | (dimethylamino)acetamido)pyridin- | | | | 1H), 8.03 (d, J = 1.7 Hz, |
| | 4-yl)-4,5-dimethyl-5- | | | | 1H), 7.77 (dd, J = 5.8, 2.1 |
| | (trifluoromethyl)tetrahydrofuran- | | | | Hz, 1H), 7.10-7.04 (m, 1H), |
| | 2-carboxamide | | | | 6.93-6.84 (m, 1H), 4.99 (d, |
| | | | | | J = 11.0 Hz, 1H), 4.07 (dd, |
| | | | | | J = 10.8, 8.1 Hz, 1H), 3.99- |
| | | | | | 3.97 (m, 3H), 3.12 (s, 2H), |
| | | | | | 2.73 (t, J = 7.7 Hz, 1H), |
| | | | | | 2.63 (s, 1H), 2.38 (s, 6H), |
| | | | | | 1.67 (s, 3H), 0.79-0.75 (m, |
| | | | | | 3H) ppm. |
| 473 | (2R,3S,4S,5R)-3-(3,4- | 529.456 | 530.16 | 2.45 | ¹H-NMR (400 MHz, |
| | difluoro-2- | | | | Chloroform-d) δ 8.50 (d, |
| | methoxyphenyl)-N-(2- | | | | J = 7.1 Hz, 1H), 8.26-8.23 |
| | ((S)-3-hydroxy-2- | | | | (m, 2H), 7.73 (td, J = 6.0, |
| | oxopyrrolidin-1- | | | | 1.9 Hz, 1H), 7.10-7.05 (m, |
| | yl)pyridin-4-yl)-4,5- | | | | 1H), 6.89 (td, J = 9.3, 7.5 |
| | dimethyl-5- | | | | Hz, 1H), 5.00 (d, J = 11.0 |
| | (trifluoromethyl)tetrahydrofuran- | | | | Hz, 1H), 4.54-4.49 (m, 1H), |
| | 2-carboxamide | | | | 4.26-4.21 (m, 1H), 4.09 |
| | | | | | (dd, J = 10.6, 8.4 Hz, 1H), |
| | | | | | 4.00 (d, J = 2.7 Hz, 3H), |
| | | | | | 3.84-3.77 (m, 1H), 2.91 (s, |
| | | | | | 1H), 2.78-2.71 (m, 1H), |
| | | | | | 2.63-2.56 (m, 1H), 2.09- |
| | | | | | 1.99 (m, 1H), 1.68 (s, 3H), |
| | | | | | 0.80-0.77 (m, 3H) ppm. |
| 474 | (2R,3S,4S,5R)-3-(3,4- | 556.525 | 557.18 | 2.49 | ¹H-NMR (400 MHz, |
| | difluoro-2- | | | | Chloroform-d) δ 8.45 (s, |
| | methoxyphenyl)-N-(2- | | | | 1H), 8.29 (d, J = 5.7 Hz, |
| | ((S)-4,5-dimethyl-2- | | | | 1H), 7.87 (d, J = 2.1 Hz, |
| | oxopiperazin-1- | | | | 1H), 7.72 (dd, J = 5.6, 1.9 |
| | yl)pyridin-4-yl)-4,5- | | | | Hz, 1H), 7.08-7.04 (m, 1H), |
| | dimethyl-5- | | | | 6.91-6.85 (m, 1H), 4.97 (d, |
| | (trifluoromethyl)tetrahydrofuran- | | | | J = 11.0 Hz, 1H), 4.08-4.02 |
| | 2-carboxamide | | | | (m, 2H), 3.99 (d, J = 2.7 |
| | | | | | Hz, 3H), 3.63-3.56 (m, 2H), |
| | | | | | 3.13 (d, J = 17.2, 1H), 2.77- |
| | | | | | 2.69 (m, 1H), 2.67-2.61 (m, |
| | | | | | 1H), 2.32 (s, 3H), 1.66 (s, |
| | | | | | 3H), 1.21 (d, J = 6.2 Hz, |
| | | | | | 3H), 0.78-0.75 (m, 3H) |
| | | | | | ppm. |
| 475 | (2R,3S,4S,5R)-3-(3,4- | 556.525 | 557.18 | 2.5 | ¹H-NMR (400 MHz, |
| | difluoro-2- | | | | Chloroform-d) δ 8.44 (s, |
| | methoxyphenyl)-N-(2- | | | | 1H), 8.29 (d, J = 5.5 Hz, |
| | ((R)-3,4-dimethyl-2- | | | | 1H), 7.85 (d, J = 1.6 Hz, |
| | oxopiperazin-1- | | | | 1H), 7.71 (dd, J = 5.6, 1.9 |
| | yl)pyridin-4-yl)-4,5- | | | | Hz, 1H), 7.09-7.04 (m, 1H), |
| | dimethyl-5- | | | | 6.92-6.85 (m, 1H), 4.97 (d, |
| | (trifluoromethyl)tetrahydrofuran- | | | | J = 11.0 Hz, 1H), 4.10-4.03 |
| | 2-carboxamide | | | | (m, 2H), 3.99 (d, J = 2.7 |
| | | | | | Hz, 3H), 3.96-3.89 (m, 1H), |
| | | | | | 3.09-2.99 (m, 2H), 2.77- |
| | | | | | 2.62 (m, 2H), 2.43 (s, 3H), |
| | | | | | 1.66 (s, 3H), 1.48 (d, J = |
| | | | | | 6.6 Hz, 3H), 0.78-0.75 (m, |
| | | | | | 3H) ppm. |
| 476 | (2R,3S,4S,5R)-3-(3,4- | 556.525 | 557.23 | 2.58 | ¹H-NMR (400 MHz, |
| | difluoro-2- | | | | Chloroform-d) δ 8.45 (s, |
| | methoxyphenyl)-N-(2- | | | | 1H), 8.29 (d, J = 5.7 Hz, |
| | ((S)-3,4-dimethyl-2- | | | | 1H), 7.87 (d, J = 1.6 Hz, |
| | oxopiperazin-1- | | | | 1H), 7.68 (dd, J = 5.7, 2.1 |
| | yl)pyridin-4-yl)-4,5- | | | | Hz, 1H), 7.08-7.04 (m, 1H), |
| | dimethyl-5- | | | | 6.88 (td, J = 9.2, 7.5 Hz, |
| | (trifluoromethyl)tetrahydrofuran- | | | | 1H), 4.97 (d, J = 11.0 Hz, |
| | 2-carboxamide | | | | 1H), 4.13-4.03 (m, 2H), |
| | | | | | 3.99 (d, J = 2.7 Hz, 3H), |
| | | | | | 3.94-3.86 (m, 1H), 3.08- |
| | | | | | 2.98 (m, 2H), 2.78-2.58 (m, |
| | | | | | 2H), 2.42 (s, 3H), 1.65 (s, |
| | | | | | 3H), 1.48 (d, J = 6.6 Hz, |
| | | | | | 3H), 0.77-0.75 (m, 3H) |
| | | | | | ppm. |

The following compounds were made using a method similar to that described in Example 11, except that different amines were used in the Buchwald coupling step 2 and General Method I was used as the final step.

| Cmpd | | LC/MS | Found | MS | |
|---|---|---|---|---|---|
| No. | Compound Name | (m/z calc.) | M + 1 | r.t. | NMR (shifts in ppm) |
| 477 | (2R,3S,4S,5R)-3-(3,4- | 542.498 | 543.13 | 2.30 | ¹H-NMR (400 MHz, |
| | difluoro-2- | | | | Chloroform-d) δ 8.50 (s, |
| | methoxyphenyl)-4,5- | | | | 1H), 8.35 (d, J = 5.5 Hz, |
| | dimethyl-N-(2-((R)-2- | | | | 1H), 7.67 (d, J = 1.6 Hz, |
| | methyl-6-oxopiperazin-1- | | | | 1H), 7.58 (dd, J = 5.6, 1.9 |
| | yl)pyridin-4-yl)-5- | | | | Hz, 1H), 7.08-7.04 (m, 1H), |
| | (trifluoromethyl)tetrahydrofuran- | | | | 6.92-6.85 (m, 1H), 4.99 (d, |
| | 2-carboxamide | | | | J = 11.0 Hz, 1H), 4.74-4.70 |
| | | | | | (m, 1H), 4.07 (dd, J = 10.9, |
| | | | | | 8.1 Hz, 1H), 4.00 (d, J = 3.0 |
| | | | | | Hz, 3H), 3.67 (dd, J = 29.8, |
| | | | | | 17.9 Hz, 2H), 3.30 (dd, J = |
| | | | | | 13.3, 4.4 Hz, 1H), 3.00 (dd, |
| | | | | | J = 13.4, 4.2 Hz, 1H), 2.77- |
| | | | | | 2.69 (m, 1H), 1.65 (s, 3H), |
| | | | | | 1.25 (s, 1H), 1.15 (d, J = |
| | | | | | 6.4 Hz, 3H), 0.78-0.76 (m, |
| | | | | | 3H) ppm. 1H not observed. |
| 478 | (2R,3S,4S,5R)-3-(3,4- | 542.498 | 543.22 | 2.37 | ¹H-NMR (400 MHz, |
| | difluoro-2- | | | | Chloroform-d) δ 8.47 (d, J = |
| | methoxyphenyl)-4,5- | | | | 6.4 Hz, 1H), 8.30 (d, J = |
| | dimethyl-N-(2-((S)-5- | | | | 5.5 Hz, 1H), 7.84 (d, J = 1.8 |
| | methyl-2-oxopiperazin-1- | | | | Hz, 1H), 7.72 (dd, J = 5.6, |
| | yl)pyridin-4-yl)-5- | | | | 1.9 Hz, 1H), 7.08-7.05 (m, |
| | (trifluoromethyl)tetrahydrofuran- | | | | 1H), 6.89 (dd, J = 16.7, 9.2 |
| | 2-carboxamide | | | | Hz, 1H), 5.0-4.97 (m, 1H), |
| | | | | | 4.20-4.15 (m, 1H), 4.06 |
| | | | | | (dd, J = 10.9, 8.1 Hz, 1H), |
| | | | | | 3.99 (d, J = 2.7 Hz, 3H), |
| | | | | | 3.80 (dd, J = 25.3, 17.7 Hz, |
| | | | | | 2H), 3.63-3.57 (m, 1H), |
| | | | | | 3.34 (s, 1H), 2.73 (t, J = 7.8 |
| | | | | | Hz, 1H), 1.66 (s, 3H), 1.33 |
| | | | | | (d, J = 6.2 Hz, 3H), 0.78- |
| | | | | | 0.76 (m, 3H) ppm. 1H not |
| | | | | | observed. |
| 479 | (2R,3S,4S,5R)-3-(3,4- | 542.498 | 543.14 | 2.34 | ¹H-NMR (400 MHz, |
| | difluoro-2- | | | | Chloroform-d) δ 8.47 (s, |
| | methoxyphenyl)-4,5- | | | | 1H), 8.34 (d, J = 5.7 Hz, |
| | dimethyl-N-(2-((S)-2- | | | | 1H), 7.74 (dd, J = 5.7, 2.1 |
| | methyl-6-oxopiperazin-1- | | | | Hz, 1H), 7.52 (d, J = 1.8 |
| | yl)pyridin-4-yl)-5- | | | | Hz, 1H), 7.09-7.05 (m, 1H), |
| | (trifluoromethyl)tetrahydrofuran- | | | | 6.89 (dd, J = 16.7, 9.2 Hz, |
| | 2-carboxamide | | | | 1H), 4.98 (d, J = 11.2 Hz, |
| | | | | | 1H), 4.77-4.70 (m, 1H), |
| | | | | | 4.06 (dd, J = 11.0, 8.2 Hz, |
| | | | | | 1H), 4.00 (d, J = 3.0 Hz, |
| | | | | | 3H), 3.68 (dd, J = 32.4, |
| | | | | | 18.0 Hz, 2H), 3.31 (dd, J = |
| | | | | | 13.4, 4.2 Hz, 1H), 3.01 (dd, |
| | | | | | J = 13.4, 4.2 Hz, 1H), 2.77- |
| | | | | | 2.70 (m, 1H), 1.66 (s, 3H), |
| | | | | | 1.14 (d, J = 6.4 Hz, 3H), |
| | | | | | 0.78-0.76 (m, 3H) ppm. 1H |
| | | | | | not observed. |
| 480 | (2R,3S,4S,5R)-3-(3,4- | 542.498 | 543.14 | 2.37 | ¹H-NMR (400 MHz, |
| | difluoro-2- | | | | Chloroform-d) δ 8.47 (s, |
| | methoxyphenyl)-4,5- | | | | 1H), 8.30 (d, J = 5.5 Hz, |
| | dimethyl-N-(2-((R)-5- | | | | 1H), 7.85 (d, J = 1.8 Hz, |
| | methyl-2-oxopiperazin-1- | | | | 1H), 7.68 (dd, J = 5.6, 1.9 |
| | yl)pyridin-4-yl)-5- | | | | Hz, 1H), 7.08-7.04 (m, 1H), |
| | (trifluoromethyl)tetrahydrofuran- | | | | 6.91-6.85 (m, 1H), 4.98 (d, |
| | 2-carboxamide | | | | J = 11.0 Hz, 1H), 4.15 (dd, |
| | | | | | J = 12.6, 3.4 Hz, 1H), 4.06 |
| | | | | | (dd, J = 10.9, 8.1 Hz, 1H), |
| | | | | | 3.99 (d, J = 2.7 Hz, 3H), |
| | | | | | 3.81-3.71 (m, 2H), 3.51 |
| | | | | | (dd, J = 12.5, 10.6 Hz, 1H), |
| | | | | | 3.31-3.23 (m, 1H), 2.76- |
| | | | | | 2.69 (m, 1H), 1.65 (s, 3H), |
| | | | | | 1.27 (d, J = 6.4 Hz, 3H), |
| | | | | | 0.78-0.76 (m, 3H) ppm. 1H |
| | | | | | not observed. |
| 481 | (2R,3S,4S,5R)-3-(3,4- | 542.498 | 543.21 | 2.37 | ¹H-NMR (400 MHz, |
| | difluoro-2- | | | | Chloroform-d) δ 8.59 (s, |
| | methoxyphenyl)-4,5- | | | | 1H), 8.29 (d, J = 5.7 Hz, |
| | dimethyl-N-(2-(2-oxo- | | | | 1H), 7.71 (dd, J = 5.7, 1.8 |
| | 1,4-diazepan-1- | | | | Hz, 1H), 7.60 (d, J = 1.8 |
| | yl)pyridin-4-yl)-5- | | | | Hz, 1H), 7.05-6.99 (m, 1H), |
| | (trifluoromethyl)tetrahydrofuran- | | | | 6.89-6.83 (m, 1H), 4.94 (d, |
| | 2-carboxamide | | | | J = 11.0 Hz, 1H), 4.23 (d, |
| | | | | | J = 6.2 Hz, 2H), 4.06 (dd, |
| | | | | | J = 10.9, 8.1 Hz, 1H), 4.00- |
| | | | | | 3.98 (m, 3H), 3.94 (s, 2H), |
| | | | | | 3.26 (t, J = 5.6 Hz, 2H), |
| | | | | | 2.75-2.67 (m, 1H), 2.61 (s, |
| | | | | | 3H), 2.04-1.99 (m, 2H), |
| | | | | | 0.76-0.74 (m, 3H) ppm. 1H |
| | | | | | not observed. |
| 482 | (2R,3S,4S,5R)-3-(3,4- | 540.482 | 541.1 | 2.46 | ¹H-NMR (400 MHz, |
| | difluoro-2- | | | | Chloroform-d) δ 8.43 (s, |
| | methoxyphenyl)-4,5- | | | | 1H), 8.19 (d, J = 5.5 Hz, |
| | dimethyl-N-(2-((1R,4R)- | | | | 1H), 7.91 (d, J = 1.8 Hz, |
| | 3-oxo-2,5- | | | | 1H), 7.67 (dd, J = 5.7, 2.1 |
| | diazabicyclo[2.2.1]heptan- | | | | Hz, 1H), 7.08-7.04 (m, 1H), |
| | 2-yl)pyridin-4-yl)-5- | | | | 6.92-6.85 (m, 1H), 5.33 (s, |
| | (trifluoromethyl)tetrahydrofuran- | | | | 1H), 4.98 (d, J = 11.0 Hz, |
| | 2-carboxamide | | | | 1H), 4.07 (dd, J = 11.0, 8.2 |
| | | | | | Hz, 1H), 3.99 (d, J = 2.7 |
| | | | | | Hz, 3H), 3.84 (s, 1H), 3.21 |
| | | | | | (dd, J = 10.1, 1.8 Hz, 1H), |
| | | | | | 2.96 (d, J = 10.1 Hz, 1H), |
| | | | | | 2.73 (t, J = 7.8 Hz, 1H), |
| | | | | | 1.96 (d, J = 9.2 Hz, 1H), |
| | | | | | 1.89 (s, 1H), 1.84 (d, J = |
| | | | | | 10.1 Hz, 1H), 1.66 (s, 3H), |
| | | | | | 0.78-0.76 (m, 3H) ppm. |
| 483 | (2R,3S,4S,5R)-3-(3,4- | 540.482 | 541.2 | 2.46 | ¹H-NMR (400 MHz, |
| | difluoro-2- | | | | Chloroform-d) δ 8.45 (s, |
| | methoxyphenyl)-4,5- | | | | 1H), 8.18 (d, J = 5.5 Hz, |
| | dimethyl-N-(2-((1S,4S)-3- | | | | 1H), 7.91 (d, J = 1.8 Hz, |
| | oxo-2,5- | | | | 1H), 7.66 (dd, J = 6.0, 1.8 |
| | diazabicyclo[2.2.1]heptan- | | | | Hz, 1H), 7.08-7.04 (m, 1H), |
| | 2-yl)pyridin-4-yl)-5- | | | | 6.88 (td, J = 9.2, 7.3 Hz, |
| | (trifluoromethyl)tetrahydrofuran- | | | | 1H), 5.32 (s, 1H), 4.97 (d, |
| | 2-carboxamide | | | | J = 11.0 Hz, 1H), 4.05 (dd, |
| | | | | | J = 11.0, 8.2 Hz, 1H), 3.98 (t, |
| | | | | | J = 3.2 Hz, 3H), 3.83-3.79 |
| | | | | | (m, 1H), 3.20 (dd, J = 9.8, |
| | | | | | 1.6 Hz, 1H), 2.96-2.93 (m, |
| | | | | | 1H), 2.73 (q, J = 7.6 Hz, |
| | | | | | 1H), 1.97-1.81 (m, 3H), |
| | | | | | 1.67 (d, J = 7.8 Hz, 3H), |
| | | | | | 0.78-0.75 (m, 3H) ppm. |
| 484 | (2R,3S,4S,5R)-3-(3,4- | 542.498 | 543.22 | 2.29 | ¹H-NMR (400 MHz, |
| | difluoro-2- | | | | Chloroform-d) δ 8.45 (s, |
| | methoxyphenyl)-4,5- | | | | 1H), 8.29 (d, J = 5.5 Hz, |
| | dimethyl-N-(2-((R)-3- | | | | 1H), 7.84 (dd, J = 10.8, 1.6 |
| | methyl-2-oxopiperazin-1- | | | | Hz, 1H), 7.69 (qd, J = 5.8, |
| | yl)pyridin-4-yl)-5- | | | | 1.9 Hz, 1H), 7.09-7.05 (m, |
| | (trifluoromethyl)tetrahydrofuran- | | | | 1H), 6.92-6.85 (m, 1H), |
| | 2-carboxamide | | | | 4.98 (d, J = 11.0 Hz, 1H), |
| | | | | | 4.08-3.96 (m, 6H), 3.74 |
| | | | | | (qd, J = 6.9, 2.9 Hz, 1H), |
| | | | | | 3.32 (td, J = 8.6, 4.2 Hz, |
| | | | | | 1H), 3.22-3.15 (m, 1H), |
| | | | | | 2.77-2.69 (m, 1H), 1.66 (s, |
| | | | | | 3H), 1.50 (d, J = 6.9 Hz, |
| | | | | | 3H), 0.78-0.76 (m, 3H) |
| | | | | | ppm. 1H not observed. |
| 485 | (2R,3S,4S,5R)-3-(3,4- | 542.498 | 543.18 | 2.27 | ¹H-NMR (400 MHz, |
| | difluoro-2- | | | | Chloroform-d) δ 8.45 (s, |
| | methoxyphenyl)-4,5- | | | | 1H), 8.29 (d, J = 5.5 Hz, |
| | dimethyl-N-(2-((S)-3- | | | | 1H), 7.83 (dd, J = 10.8, 1.8 |
| | methyl-2-oxopiperazin-1- | | | | Hz, 1H), 7.68 (qd, J = 5.7, |
| | yl)pyridin-4-yl)-5- | | | | 2.0 Hz, 1H), 7.09-7.05 (m, |
| | (trifluoromethyl)tetrahydrofuran- | | | | 1H), 6.92-6.85 (m, 1H), |
| | 2-carboxamide | | | | 4.98 (d, J = 11.0 Hz, 1H), |
| | | | | | 4.08-3.94 (m, 6H), 3.72 |
| | | | | | (qd, J = 6.9, 3.0 Hz, 1H), |
| | | | | | 3.31 (td, J = 8.6, 4.3 Hz, |
| | | | | | 1H), 3.20-3.14 (m, 1H), |
| | | | | | 2.77-2.69 (m, 1H), 1.66 (s, |
| | | | | | 3H), 1.49 (d, J = 6.9 Hz, |
| | | | | | 3H), 0.78-0.76 (m, 3H) |
| | | | | | ppm. 1H not observed. |
| 486 | (2R,3S,4S,5R)-3-(3,4- | 530.488 | 531.17 | 2.32 | ¹H-NMR (400 MHz, |
| | difluoro-2- | | | | Chloroform-d) δ 8.56 (s, |
| | methoxyphenyl)-4,5- | | | | 1H), 8.33 (d, J = 5.5 Hz, |
| | dimethyl-N-(2-(N-methyl- | | | | 1H), 7.61 (d, J = 1.8 Hz, |
| | 2-(methylamino)acetamido) | | | | 1H), 7.40 (d, J = 4.1 Hz, |
| | pyridin-4-yl)-5- | | | | 1H), 7.08-7.04 (m, 1H), |
| | (trifluoromethyl)tetrahydrofuran- | | | | 6.89 (td, J = 9.2, 7.6 Hz, |
| | 2-carboxamide | | | | 1H), 5.00 (d, J = 11.0 Hz, |
| | | | | | 1H), 4.08 (dd, J = 11.0, 7.8 |
| | | | | | Hz, 1H), 4.00 (d, J = 2.7 |
| | | | | | Hz, 3H), 3.38 (d, J = 6.9 |
| | | | | | Hz, 2H), 3.34 (d, J = 4.6 |
| | | | | | Hz, 3H), 2.74 (t, J = 7.8 Hz, |
| | | | | | 1H), 2.38 (s, 3H), 1.67 (s, |
| | | | | | 3H), 0.79-0.76 (m, 3H) |
| | | | | | ppm. One proton not |
| | | | | | located. |
| 487 | (2R,3S,4S,5R)-3-(3,4- | 530.488 | 531.2 | 2.41 | ¹H-NMR (400 MHz, |
| | difluoro-2- | | | | Chloroform-d) δ 8.34 (s, |
| | methoxyphenyl)-4,5- | | | | 1H), 8.04 (d, J = 6.0 Hz, |
| | dimethyl-N-(2-(methyl(2- | | | | 1H), 7.08-7.02 (m, 2H), |
| | (methylamino)-2- | | | | 6.93-6.86 (m, 1H), 6.65 |
| | oxoethyl)amino)pyridin- | | | | (dd, J = 5.5, 1.8 Hz, 1H), |
| | 4-yl)-5- | | | | 6.46 (s, 1H), 4.98 (d, J = |
| | (trifluoromethyl)tetrahydrofuran- | | | | 10.5 Hz, 1H), 4.19 (s, 2H), |
| | 2-carboxamide | | | | 4.08 (dd, J = 10.8, 8.0 Hz, |
| | | | | | 1H), 3.99 (d, J = 3.2 Hz, |
| | | | | | 3H), 3.03 (d, J = 18.3 Hz, |
| | | | | | 3H), 2.77 (d, J = 4.6 Hz, |
| | | | | | 3H), 2.73 (t, J = 7.8 Hz, |
| | | | | | 1H), 1.67 (s, 3H), 0.79-0.76 |
| | | | | | (m, 3H) ppm. |
| 488 | (2R,3S,4S,5R)-3-(3,4- | 516.461 | 517.21 | 2.45 | ¹H-NMR (400 MHz, |
| | difluoro-2- | | | | Chloroform-d) δ 9.75 (s, |
| | methoxyphenyl)-4,5- | | | | 1H), 8.48 (s, 1H), 8.18 (d, |
| | dimethyl-N-(2-((2- | | | | J = 5.5 Hz, 1H), 8.03 (d, J = |
| | (methylamino)-2- | | | | 1.8 Hz, 1H), 7.76 (dd, J = |
| | oxoethyl)amino)pyridin- | | | | 6.0, 1.8 Hz, 1H), 7.09-7.05 |
| | 4-yl)-5- | | | | (m, 1H), 6.88 (td, J = 9.2, |
| | (trifluoromethyl)tetrahydrofuran- | | | | 7.6 Hz, 1H), 4.98 (d, J = |
| | 2-carboxamide | | | | 11.0 Hz, 1H), 4.06 (dd, J = |
| | | | | | 10.5, 8.2 Hz, 1H), 3.98 (t, |
| | | | | | J = 3.2 Hz, 3H), 3.37 (s, 2H), |
| | | | | | 2.73 (t, J = 7.8 Hz, 1H), |
| | | | | | 2.49 (s, 3H), 1.67 (s, 3H), |
| | | | | | 0.78-0.76 (m, 3H) ppm one |
| | | | | | proton not located. |

The following compounds were made using a method similar to that described in Example 11, except that different amines were used in the Buchwald coupling step 2. Boc deprotection using General Method I, followed by methylation using General Method K, with DCM as solvent, were used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 489 | (2R,3S,4S,5R)-3-(3,4- | 554.50 | 555.2 | 2.56 | ¹H-NMR (400 MHz, |
| | difluoro-2- | | | | Chloroform-d) δ 8.43 (s, 1H), |
| | methoxyphenyl)-4,5- | | | | 8.18 (d, J = 5.5 Hz, 1H), 7.95 |
| | dimethyl-N-(2-((1S,4S)- | | | | (d, J = 1.8 Hz, 1H), 7.70 (dd, |
| | 5-methyl-3-oxo-2,5- | | | | J = 5.5, 1.8 Hz, 1H), 7.09- |
| | diazabicyclo[2.2.1]heptan- | | | | 7.05 (m, 1H), 6.88 (dd, J = |
| | 2-yl)pyridin-4-yl)-5- | | | | 16.5, 9.2 Hz, 1H), 5.23 (s, |
| | (trifluoromethyl)tetrahydrofuran- | | | | 1H), 4.97 (d, J = 11.0 Hz, |
| | 2-carboxamide | | | | 1H), 4.06 (dd, J = 11.0, 8.7 |
| | | | | | Hz, 1H), 3.99 (d, J = 2.7 Hz, |
| | | | | | 3H), 3.59 (s, 1H), 3.46 (dd, |
| | | | | | J = 9.6, 1.4 Hz, 1H), 2.73 (t, |
| | | | | | J = 7.8 Hz, 1H), 2.43 (s, 3H), |
| | | | | | 2.27 (d, J = 9.6 Hz, 1H), |
| | | | | | 2.01-1.95 (m, 2H), 1.68 (s, |
| | | | | | 3H), 0.78-0.76 (m, 3H) ppm. |
| 490 | (2R,3S,4S,5R)-3-(3,4- | 554.509 | 555.2 | 2.6 | ¹H-NMR (400 MHz, |
| | difluoro-2- | | | | Chloroform-d) δ 8.46 (s, 1H), |
| | methoxyphenyl)-4,5- | | | | 8.17 (d, J = 5.5 Hz, 1H), 7.96 |
| | dimethyl-N-(2-((1R,4R)- | | | | (d, J = 1.8 Hz, 1H), 7.69 (dd, |
| | 5-methyl-3-oxo-2,5- | | | | J = 5.7, 2.1 Hz, 1H), 7.08- |
| | diazabicyclo[2.2.1]heptan- | | | | 7.04 (m, 1H), 6.91-6.85 (m, |
| | 2-yl)pyridin-4-yl)-5- | | | | 1H), 5.23 (s, 1H), 4.97 (d, J = |
| | (trifluoromethyl)tetrahydrofuran- | | | | 11.0 Hz, 1H), 4.07 (dd, J = |
| | 2-carboxamide | | | | 11.0, 8.2 Hz, 1H), 3.99 (d, |
| | | | | | J = 2.7 Hz, 3H), 3.57 (d, J = |
| | | | | | 1.4 Hz, 1H), 3.44 (dd, J = |
| | | | | | 9.6, 1.8 Hz, 1H), 2.73 (t, J = |
| | | | | | 7.8 Hz, 1H), 2.42 (s, 3H), |
| | | | | | 2.24 (d, J = 9.6 Hz, 1H), |
| | | | | | 1.96-1.93 (m, 2H), 1.66 (s, |
| | | | | | 3H), 0.78-0.75 (m, 3H) ppm. |

The following compounds were made using a method similar to that described in Example 11, except that different amides were used in coupling step 2. Diastereomeric products of step 2 were separated by chiral SFC and General Method I was used as the final step on separated isomers. In the

| Cmpd | | LC/MS | Found | MS | |
|---|---|---|---|---|---|
| No. | Compound Name | (m/z calc.) | M + 1 | r.t. | NMR (shifts in ppm) |
| 491 | rel-(2R*,3S*,4S*,5R*)- | 540.482 | 541 | 3.2 | ¹H NMR (500 MHz, DMSO- |
| | 3-(3,4-difluoro-2- | | | | d₆) δ 10.71 (s, 1H), 8.49 (d, |
| | methoxyphenyl)-4,5- | | | | J = 1.9 Hz, 1H), 8.28 (d, J = |
| | dimethyl-N-(2-((1S,5R)- | | | | 5.6 Hz, 1H), 7.55 (dd, J = |
| | 2-oxo-3,6- | | | | 5.7, 1.9 Hz, 1H), 7.28-7.02 |
| | diazabicyclo[3.1.1]heptan- | | | | (m, 2H), 5.11 (d, J = 10.2 Hz, |
| | 3-yl)pyridin-4-yl)-5- | | | | 1H), 4.26 (dd, J = 10.2, 7.7 |
| | (trifluoromethyl)tetrahydrofuran- | | | | Hz, 1H), 3.98 (dd, J = 18.1, |
| | 2-carboxamide | | | | 1.9 Hz, 6H), 3.56 (dd, J = |
| | (precusor was first | | | | 5.9, 4.1 Hz, 1H), 2.83-2.73 |
| | eluting isomer by SFC | | | | (m, 2H), 2.68 (dt, J = 8.7, 6.1 |
| | using Chiralpak IC | | | | Hz, 1H), 1.73 (t, J = 8.1 Hz, |
| | column) | | | | 1H), 1.60 (s, 3H), 0.74 (d, J = |
| | | | | | 7.4 Hz, 3H) ppm. |
| 492 | rel-(2R*,3S*,4S*,5R*)- | 540.482 | 541 | 3.21 | ¹H NMR (500 MHz, DMSO- |
| | 3-(3,4-difluoro-2- | | | | d₆) δ 10.71 (s, 1H), 8.50 (d, |
| | methoxyphenyl)-4,5- | | | | J = 1.9 Hz, 1H), 8.28 (d, J = |
| | dimethyl-N-(2-((1R,5S)- | | | | 5.6 Hz, 1H), 7.55 (dd, J = |
| | 2-oxo-3,6- | | | | 5.6, 1.9 Hz, 1H), 7.18 (dd, |
| | diazabicyclo[3.1.1]heptan- | | | | J = 9.7, 7.5 Hz, 1H), 7.12 (d, |
| | 3-yl)pyridin-4-yl)-5- | | | | J = 5.8 Hz, 1H), 5.11 (d, J = |
| | (trifluoromethyl)tetrahydrofuran- | | | | 10.3 Hz, 1H), 4.26 (dd, J = |
| | 2-carboxamide | | | | 10.2, 7.6 Hz, 1H), 4.12-3.85 |
| | (precusor was second | | | | (m, 6H), 3.56 (dd, J = 6.0, |
| | eluting isomer by SFC | | | | 4.2 Hz, 1H), 2.92-2.72 (m, |
| | using Chiralpak IC | | | | 2H), 2.68 (dt, J = 8.7, 6.2 Hz, |
| | column) | | | | 1H), 1.73 (t, J = 8.1 Hz, 1H), |
| | | | | | 1.60 (s, 3H), 0.79-0.68 (m, |
| | | | | | 3H) ppm. |
| 493 | rel-(2R*,3S*,4S*,5R*)- | 554.509 | 555 | 3.17 | ¹H NMR (500 MHz, DMSO- |
| | 3-(3,4-difluoro-2- | | | | d₆) δ 10.65 (s, 1H), 8.26 (d, |
| | methoxyphenyl)-4,5- | | | | J = 5.6 Hz, 1H), 8.14 (d, J = |
| | dimethyl-N-(2-((1S,5R)- | | | | 1.9 Hz, 1H), 7.51 (dd, J = |
| | 2-oxo-3,8- | | | | 5.6, 1.9 Hz, 1H), 7.17 (dd, |
| | diazabicyclo[3.2.1]octan- | | | | J = 9.7, 7.6 Hz, 1H), 7.12 (d, |
| | 3-yl)pyridin-4-yl)-5- | | | | J = 7.0 Hz, 1H), 5.09 (d, J = |
| | (trifluoromethyl)tetrahydrofuran- | | | | 10.3 Hz, 1H), 4.25 (dd, J = |
| | 2-carboxamide | | | | 10.2, 7.7 Hz, 1H), 3.96 (d, |
| | (precusor was first | | | | J = 2.0 Hz, 3H), 3.83 (dd, J = |
| | eluting isomer by SFC | | | | 11.8, 4.3 Hz, 1H), 3.77 (s, |
| | using Lux Cellulose-2 | | | | 1H), 3.66 (d, J = 3.9 Hz, 1H), |
| | column) | | | | 3.55 (d, J = 11.8 Hz, 1H), |
| | | | | | 3.12 (s, 1H), 2.77 (p, J = 7.7 |
| | | | | | Hz, 1H), 1.90 (t, J = 3.5 Hz, |
| | | | | | 3H), 1.70 (d, J = 19.8 Hz, |
| | | | | | 1H), 1.60 (s, 3H), 0.73 (d, J = |
| | | | | | 7.3 Hz, 3H) ppm. |
| 494 | rel-(2R*,3S*,4S*,5R*)- | 554.509 | 555 | 3.17 | ¹H NMR (500 MHz, DMSO- |
| | 3-(3,4-difluoro-2- | | | | d₆) δ 10.64 (s, 1H), 8.26 (d, |
| | methoxyphenyl)-4,5- | | | | J = 5.6 Hz, 1H), 8.13 (d, J = |
| | dimethyl-N-(2-((1R,5S)- | | | | 1.9 Hz, 1H), 7.50 (dd, J = |
| | 2-oxo-3,8- | | | | 5.7, 1.9 Hz, 1H), 7.30-7.01 |
| | diazabicyclo[3.2.1]octan- | | | | (m, 2H), 5.09 (d, J = 10.2 Hz, |
| | 3-yl)pyridin-4-yl)-5- | | | | 1H), 4.24 (dd, J = 10.3, 7.7 |
| | (trifluoromethyl)tetrahydrofuran- | | | | Hz, 1H), 3.96 (d, J = 2.0 Hz, |
| | 2-carboxamide | | | | 3H), 3.86-3.75 (m, 2H), |
| | (precusor was second | | | | 3.66 (s, 1H), 3.58 (d, J = 11.4 |
| | eluting isomer by SFC | | | | Hz, 1H), 3.11 (s, 1H), 2.77 |
| | using Lux Cellulose-2 | | | | (p, J = 7.4 Hz, 1H), 1.90 (t, |
| | column) | | | | J = 2.9 Hz, 3H), 1.72-1.64 |
| | | | | | (m, 1H), 1.60 (s, 3H0.73 (d, |
| | | | | | J = 7.1 Hz, 3H) ppm. |
| 495 | rel-(2R*,3S*,4S*,5R*)- | 528.472 | 529 | 3.13 | ¹H NMR (500 MHz, DMSO- |
| | N-(2-(4-amino-2- | | | | d₆) δ 10.72 (s, 1H), 8.49 (d, |
| | oxopyrrolidin-1- | | | | J = 1.9 Hz, 1H), 8.23 (d, J = |
| | yl)pyridin-4-yl)-3-(3,4- | | | | 5.7 Hz, 1H), 7.52 (dd, J = |
| | difluoro-2- | | | | 5.7, 1.9 Hz, 1H), 7.21-7.15 |
| | methoxyphenyl)-4,5- | | | | (m, 1H), 7.13-7.08 (m, 1H), |
| | dimethyl-5- | | | | 5.10 (d, J = 10.2 Hz, 1H), |
| | (trifluoromethyl)tetrahydrofuran- | | | | 4.25 (dd, J = 10.2, 7.7 Hz, |
| | 2-carboxamide | | | | 1H), 4.00 (dd, J = 11.1, 5.9 |
| | (precusor was first | | | | Hz, 1H), 3.96 (d, J = 2.1 Hz, |
| | eluting isomer by SFC | | | | 3H), 3.67 (dd, J = 11.1, 3.2 |
| | using Whelk-O1 | | | | Hz, 1H), 3.60 (td, J = 6.6, 3.4 |
| | column) | | | | Hz, 1H), 2.91-2.71 (m, 2H), |
| | | | | | 2.24 (dd, J = 16.7, 3.7 Hz, |
| | | | | | 1H), 1.80 (s, 2H), 1.59 (s, |
| | | | | | 3H), 0.73 (d, J = 7.1 Hz, 3H) |
| | | | | | ppm. |
| 496 | rel-(2R*,3S*,4S*,5R*)- | 528.472 | 529 | 3.13 | ¹H NMR (500 MHz, DMSO- |
| | N-(2-(4-amino-2- | | | | d₆) δ 10.71 (s, 1H), 8.50 (d, |
| | oxopyrrolidin-1- | | | | J = 1.9 Hz, 1H), 8.23 (d, J = |
| | yl)pyridin-4-yl)-3-(3,4- | | | | 5.7 Hz, 1H), 7.50 (dd, J = |
| | difluoro-2- | | | | 5.7, 1.9 Hz, 1H), 7.26-7.03 |
| | methoxyphenyl)-4,5- | | | | (m, 2H), 5.10 (d, J = 10.2 Hz, |
| | dimethyl-5- | | | | 1H), 4.25 (dd, J = 10.3, 7.7 |
| | (trifluoromethyl)tetrahydrofuran- | | | | Hz, 1H), 3.99 (dd, J = 11.1, |
| | 2-carboxamide | | | | 6.0 Hz, 1H), 3.96 (d, J = 2.1 |
| | (precusor was second | | | | Hz, 3H), 3.68 (dd, J = 11.1, |
| | eluting isomer by SFC | | | | 3.3 Hz, 1H), 3.63-3.57 (m, |
| | using Whelk-O1 | | | | 1H), 2.87-2.73 (m, 2H), |
| | column) | | | | 2.24 (dd, J = 16.7, 3.8 Hz, |
| | | | | | 1H), 1.80 (s, 2H), 1.60 (s, |
| | | | | | 3H), 0.73 (d, J = 6.9 Hz, 3H) |
| | | | | | ppm. |
| 497 | rel-(2R*,3S*,4S*,5R*)- | 528.472 | 529 | 3.18 | ¹H NMR (500 MHz, DMSO- |
| | N-(2-(3-amino-2- | | | | d₆) δ 10.74 (s, 1H), 8.54 (d, |
| | oxopyrrolidin-1- | | | | J = 1.8 Hz, 1H), 8.25 (d, J = |
| | yl)pyridin-4-yl)-3-(3,4- | | | | 5.7 Hz, 1H), 7.51 (dd, J = |
| | difluoro-2- | | | | 5.7, 1.9 Hz, 1H), 7.26-7.04 |
| | methoxyphenyl)-4,5- | | | | (m, 2H), 5.10 (d, J = 10.2 Hz, |
| | dimethyl-5- | | | | 1H), 4.26 (dd, J = 10.2, 7.7 |
| | (trifluoromethyl)tetrahydrofuran- | | | | Hz, 1H), 4.05-3.97 (m, 1H), |
| | 2-carboxamide | | | | 3.96 (d, J = 2.1 Hz, 3H), 3.68 |
| | (precusor was first | | | | (td, J = 10.4, 6.7 Hz, 1H), |
| | eluting isomer by SFC | | | | 3.60 (t, J = 9.3 Hz, 1H), 2.78 |
| | using Whelk-O1 | | | | (p, J = 7.6 Hz, 1H), 2.33 (dt, |
| | column) | | | | J = 11.3, 8.0 Hz, 1H), 1.90 (s, |
| | | | | | 2H), 1.70 (dt, J = 20.8, 10.0 |
| | | | | | Hz, 1H), 1.60 (s, 3H), 0.73 |
| | | | | | (d, J = 7.4 Hz, 3H) ppm. |
| 498 | rel-(2R*,3S*,4S*,5R*)- | 528.472 | 529 | 3.18 | ¹H NMR (500 MHz, DMSO- |
| | N-(2-(3-amino-2- | | | | d₆) δ 10.72 (s, 1H), 8.55 (d, |
| | oxopyrrolidin-1- | | | | J = 1.9 Hz, 1H), 8.25 (d, J = |
| | yl)pyridin-4-yl)-3-(3,4- | | | | 5.6 Hz, 1H), 7.50 (dd, J = |
| | difluoro-2- | | | | 5.7, 1.9 Hz, 1H), 7.24-7.06 |
| | methoxyphenyl)-4,5- | | | | (m, 2H), 5.10 (d, J = 10.2 Hz, |
| | dimethyl-5- | | | | 1H), 4.26 (dd, J = 10.2, 7.7 |
| | (trifluoromethyl)tetrahydrofuran- | | | | Hz, 1H), 4.02 (ddd, J = 10.8, |
| | 2-carboxamide | | | | 8.9, 1.8 Hz, 1H), 3.96 (d, J = |
| | (precusor was second | | | | 2.0 Hz, 3H), 3.67 (td, J = |
| | eluting isomer by SFC | | | | 10.4, 6.8 Hz, 1H), 3.60 (t, J = |
| | using Whelk-O1 | | | | 9.3 Hz, 1H), 2.78 (p, J = 7.5 |
| | column) | | | | Hz, 1H), 2.35-2.29 (m, 1H), |
| | | | | | 1.90 (s, 2H), 1.79-1.65 (m, |
| | | | | | 1H), 1.60 (s, 3H), 0.73 (d, J = |
| | | | | | 7.4 Hz, 3H) ppm. |
| 499 | rel-(2R*,3S*,4S*,5R*)- | 542.498 | 543 | 3.23 | ¹H NMR (500 MHz, DMSO- |
| | 3-(3,4-difluoro-2- | | | | d₆) δ 10.72 (s, 1H), 8.49 (d, |
| | methoxyphenyl)-4,5- | | | | J = 1.8 Hz, 1H), 8.23 (d, J = |
| | dimethyl-N-(2-(4- | | | | 5.7 Hz, 1H), 7.52 (dd, J = |
| | (methylamino)-2- | | | | 5.7, 1.9 Hz, 1H), 7.22-7.15 |
| | oxopyrrolidin-1- | | | | (m, 1H), 7.11 (dd, J = 8.5, |
| | yl)pyridin-4-yl)-5- | | | | 6.5 Hz, 1H), 5.09 (d, J = 10.3 |
| | (trifluoromethyl)tetrahydrofuran- | | | | Hz, 1H), 4.25 (dd, J = 10.2, |
| | 2-carboxamide | | | | 7.6 Hz, 1H), 4.00 (dd, J = |
| | (precusor was first | | | | 11.4, 6.1 Hz, 1H), 3.96 (d, |
| | eluting isomer by SFC | | | | J = 2.1 Hz, 3H), 3.79 (dd, J = |
| | using Whelk-O1 | | | | 11.4, 3.1 Hz, 1H), 3.26 (tt, |
| | column) | | | | J = 6.7, 3.4 Hz, 1H), 2.79 (ddd, |
| | | | | | J = 17.2, 16.0, 7.2 Hz, 2H), |
| | | | | | 2.35 (dd, J = 16.8, 3.6 Hz, |
| | | | | | 1H), 2.27 (s, 3H), 1.59 (s, |
| | | | | | 3H), 0.79-0.65 (m, 3H) |
| | | | | | ppm; 1H not observed |
| 500 | rel-(2R*,3S*,4S*,5R*)- | 542.498 | 543 | 3.23 | ¹H NMR (500 MHz, DMSO- |
| | 3-(3,4-difluoro-2- | | | | d₆) δ 10.71 (s, 1H), 8.50 (d, |
| | methoxyphenyl)-4,5- | | | | J = 1.9 Hz, 1H), 8.23 (d, J = |
| | dimethyl-N-(2-(4- | | | | 5.7 Hz, 1H), 7.51 (dd, J = |
| | (methylamino)-2- | | | | 5.7, 1.9 Hz, 1H), 7.28-7.15 |
| | oxopyrrolidin-1- | | | | (m, 1H), 7.11 (t, J = 7.3 Hz, |
| | yl)pyridin-4-yl)-5- | | | | 1H), 5.09 (d, J = 10.3 Hz, |
| | (trifluoromethyl)tetrahydrofuran- | | | | 1H), 4.25 (dd, J = 10.2, 7.7 |
| | 2-carboxamide | | | | Hz, 1H), 3.99 (dd, J = 11.3, |
| | (precusor was second | | | | 6.2 Hz, 1H), 3.96 (d, J = 2.1 |
| | eluting isomer by SFC | | | | Hz, 3H), 3.80 (dd, J = 11.4, |
| | using Whelk-O1 | | | | 3.2 Hz, 1H), 3.26 (dp, J = |
| | column) | | | | 6.7, 3.4 Hz, 1H), 2.87-2.71 |
| | | | | | (m, 2H), 2.35 (dd, J = 16.9, |
| | | | | | 3.7 Hz, 1H), 2.27 (s, 3H), |
| | | | | | 1.59 (s, 3H), 0.73 (d, J = 7.4 |
| | | | | | Hz, 3H) ppm; 1H not |
| | | | | | observed. |
| 501 | rel-(2R*,3S*,4S*,5R*)- | 542.498 | 543 | 3.31 | ¹H NMR (500 MHz, DMSO- |
| | 3-(3,4-difluoro-2- | | | | d₆) δ 10.73 (s, 1H), 8.53 (d, |
| | methoxyphenyl)-4,5- | | | | J = 1.9 Hz, 1H), 8.25 (d, J = |
| | dimethyl-N-(2-(3- | | | | 5.6 Hz, 1H), 7.52 (dd, J = |
| | (methylamino)-2- | | | | 5.6, 1.9 Hz, 1H), 7.29-7.16 |
| | oxopyrrolidin-1- | | | | (m, 1H), 7.14-6.99 (m, 1H), |
| | yl)pyridin-4-yl)-5- | | | | 5.10 (d, J = 10.2 Hz, 1H), |
| | (trifluoromethyl)tetrahydrofuran- | | | | 4.26 (dd, J = 10.2, 7.6 Hz, |
| | 2-carboxamide | | | | 1H), 4.01 (ddd, J = 11.2, 8.8, |
| | (precursor was first | | | | 2.5 Hz, 1H), 3.96 (d, J = 2.1 |
| | eluting isomer by SFC | | | | Hz, 3H), 3.74 (ddd, J = 11.0, |
| | using Whelk-O1 | | | | 9.2, 7.1 Hz, 1H), 3.48 (dd, |
| | column) | | | | J = 9.4, 8.1 Hz, 1H), 2.78 (p, |
| | | | | | J = 7.4 Hz, 1H), 2.37 (s, 3H), |
| | | | | | 2.34-2.26 (m, 1H), 2.18 (br |
| | | | | | s, 1H), 1.75 (dq, J = 12.2, 8.9 |
| | | | | | Hz, 1H), 1.60 (s, 3H), 0.79- |
| | | | | | 0.68 (m, 3H) ppm. |
| 502 | rel-(2R*,3S*,4S*,5R*)- | 542.498 | 543 | 3.31 | ¹H NMR (500 MHz, DMSO- |
| | 3-(3,4-difluoro-2- | | | | d₆) δ 10.71 (s, 1H), 8.55 (d, |
| | methoxyphenyl)-4,5- | | | | J = 1.9 Hz, 1H), 8.25 (d, J = |
| | dimethyl-N-(2-(3- | | | | 5.7 Hz, 1H), 7.50 (dd, J = |
| | (methylamino)-2- | | | | 5.7, 2.0 Hz, 1H), 7.18 (q, J = |
| | oxopyrrolidin-1- | | | | 9.0 Hz, 1H), 7.12 (t, J = 7.4 |
| | yl)pyridin-4-yl)-5- | | | | Hz, 1H), 5.10 (d, J = 10.2 |
| | (trifluoromethyl)tetrahydrofuran- | | | | Hz, 1H), 4.26 (dd, J = 10.2, |
| | 2-carboxamide | | | | 7.7 Hz, 1H), 4.03 (ddd, J = |
| | (precursor was second | | | | 11.2, 8.8, 2.6 Hz, 1H), 3.96 |
| | eluting isomer by SFC | | | | (d, J = 2.1 Hz, 3H), 3.73 |
| | using Whelk-O1 | | | | (ddd, J = 11.0, 9.0, 7.0 Hz, |
| | column) | | | | 1H), 3.48 (t, J = 8.7 Hz, 1H), |
| | | | | | 2.78 (p, J = 7.6 Hz, 1H), 2.37 |
| | | | | | (s, 3H), 2.34-2.26 (m, 1H), |
| | | | | | 2.17 (br s, 1H), 1.75 (dq, J = |
| | | | | | 12.2, 9.0 Hz, 1H), 1.60 (s, |
| | | | | | 3H), 0.73 (d, J = 7.3 Hz, 3H) |
| | | | | | ppm. |

The following compounds were made using a method similar to that described in Example 11, except that different amides were used in coupling step 2. Diastereomeric products of step 2 were separated by chiral SFC and General Method I, followed by General Method K, with DCM as solvent, were used as the final steps on separated isomers. In the Table below, “MS r.t.” stands for Mass Spec

| Cmpd | | LC/MS | Found | MS | |
|---|---|---|---|---|---|
| No. | Compound Name | (m/z calc.) | M + 1 | r.t. | NMR (shifts in ppm) |
| 503 | rel-(2R*,3S*,4S*,5R*)- | 568.536 | 569.2 | 3.29 | ¹H NMR (500 MHz, DMSO- |
| | 3-(3,4-difluoro-2- | | | | d₆) δ 10.65 (s, 1H), 8.28 (d, |
| | methoxyphenyl)-4,5- | | | | J = 5.5 Hz, 1H), 8.11 (d, J = |
| | dimethyl-N-(2-((1S,5R)- | | | | 1.9 Hz, 1H), 7.52 (dd, J = |
| | 8-methyl-2-oxo-3,8- | | | | 5.6, 1.9 Hz, 1H), 7.27-7.02 |
| | diazabicyclo[3.2.1]octan-3- | | | | (m, 2H), 5.09 (d, J = 10.2 Hz, |
| | yl)pyridin-4-yl)-5- | | | | 1H), 4.25 (dd, J = 10.3, 7.7 |
| | (trifluoromethyl)tetrahydrofuran- | | | | Hz, 1H), 3.99 (dd, J = 12.3, |
| | 2-carboxamide | | | | 4.3 Hz, 1H), 3.96 (d, J = 2.1 |
| | (precursor was first | | | | Hz, 3H), 3.50 (dd, J = 11.7, |
| | eluting isomer by SFC | | | | 6.7 Hz, 2H), 3.43 (d, J = 6.5 |
| | using Lux Cellulose-2 | | | | Hz, 1H), 2.77 (p, J = 7.6 Hz, |
| | column) | | | | 1H), 2.36 (s, 4H), 2.22-2.05 |
| | | | | | (m, 1H), 1.92-1.80 (m, 1H), |
| | | | | | 1.77-1.66 (m, 1H), 1.60 (s, |
| | | | | | 3H), 0.73 (d, J = 7.3 Hz, 3H) |
| | | | | | ppm. |
| 504 | rel-(2R*,3S*,4S*,5R*)- | 568.536 | 569.2 | 3.29 | ¹H NMR (500 MHz, DMSO- |
| | 3-(3,4-difluoro-2- | | | | d₆) δ 10.65 (s, 1H), 8.27 (d, |
| | methoxyphenyl)-4,5- | | | | J = 5.7 Hz, 1H), 8.11 (s, 1H), |
| | dimethyl-N-(2-((1R,5S)- | | | | 7.51 (d, J = 5.6 Hz, 1H), 7.30- |
| | 8-methyl-2-oxo-3,8- | | | | 7.06 (m, 2H), 5.08 (d, J = |
| | diazabicyclo[3.2.1]octan- | | | | 10.3 Hz, 1H), 4.24 (dd, J = |
| | 3-yl)pyridin-4-yl)-5- | | | | 10.3, 7.7 Hz, 1H), 4.10-3.82 |
| | (trifluoromethyl)tetrahydrofuran- | | | | (m, 4H), 3.67-3.40 (m, 3H), |
| | 2-carboxamide | | | | 2.77 (p, J = 7.4 Hz, 1H), 2.37 |
| | (precursor was second | | | | (s, 3H), 2.22-2.04 (m, 2H), |
| | eluting isomer by SFC | | | | 1.91-1.78 (m, 1H), 1.71 (dt, |
| | using Lux Cellulose-2 | | | | J = 12.5, 5.5 Hz, 1H), 1.60 (s, |
| | column) | | | | 3H), 0.73 (d, J = 7.4 Hz, 3H) |
| | | | | | ppm. |
| 505 | rel-(2R*,3S*,4S*,5R*)- | 556.525 | 557.2 | 3.36 | ¹H NMR (500 MHz, DMSO- |
| | 3-(3,4-difluoro-2- | | | | d₆) δ 10.72 (s, 1H), 8.48 (s, |
| | methoxyphenyl)-N-(2- | | | | 1H), 8.24 (d, J = 5.6 Hz, 1H), |
| | (4-(dimethylamino)-2- | | | | 7.51 (d, J = 5.7 Hz, 1H), 7.26- |
| | oxopyrrolidin-1- | | | | 7.04 (m, 2H), 5.09 (d, J = |
| | yl)pyridin-4-yl)-4,5- | | | | 10.3 Hz, 1H), 4.25 (dd, J = |
| | dimethyl-5- | | | | 10.2, 7.7 Hz, 1H), 4.13 (dd, |
| | (trifluoromethyl)tetrahydrofuran- | | | | J = 11.1, 7.4 Hz, 1H), 3.96 (d, |
| | 2-carboxamide | | | | J = 2.1 Hz, 3H), 3.71 (dd, J = |
| | (precursor was first | | | | 11.1, 6.4 Hz, 1H), 3.02 (p, |
| | eluting isomer by SFC | | | | J = 7.3 Hz, 1H), 2.85-2.68 |
| | using Whelk-O1 | | | | (m, 2H), 2.61-2.53 (m, 1H), |
| | column) | | | | 2.16 (s, 6H), 1.59 (s, 3H), |
| | | | | | 0.73 (d, J = 7.2 Hz, 3H) ppm. |
| 506 | rel-(2R*,3S*,4S*,5R*)- | 556.525 | 557.2 | 3.36 | ¹H NMR (500 MHz, DMSO- |
| | 3-(3,4-difluoro-2- | | | | d₆) δ 10.73 (s, 1H), 8.48 (s, |
| | methoxyphenyl)-N-(2- | | | | 1H), 8.24 (d, J = 5.8 Hz, 1H), |
| | (4-(dimethylamino)-2- | | | | 7.51 (d, J = 5.7 Hz, 1H), 7.33- |
| | oxopyrrolidin-1- | | | | 7.02 (m, 2H), 5.09 (d, J = |
| | yl)pyridin-4-yl)-4,5- | | | | 10.3 Hz, 1H), 4.29-4.20 (m, |
| | dimethyl-5- | | | | 1H), 4.12 (dd, J = 11.1, 7.3 |
| | (trifluoromethyl)tetrahydrofuran- | | | | Hz, 1H), 3.96 (d, J = 2.1 Hz, |
| | 2-carboxamide | | | | 3H), 3.72 (dd, J = 11.1, 6.5 |
| | (precursor was second | | | | Hz, 1H), 3.02 (p, J = 7.5 Hz, |
| | eluting isomer by SFC | | | | 1H), 2.85-2.68 (m, 2H), |
| | using Whelk-O1 | | | | 2.61-2.53 (m, 1H), 2.16 (s, |
| | column) | | | | 6H), 1.59 (s, 3H), 0.73 (d, J = |
| | | | | | 7.4 Hz, 3H) ppm. |
| 507 | rel-(2R*,3S*,4S*,5R*)- | 556.525 | 557.2 | 3.43 | ¹H NMR (500 MHz, DMSO- |
| | 3-(3,4-difluoro-2- | | | | d₆) δ 10.73 (s, 1H), 8.56 (d, |
| | methoxyphenyl)-N-(2- | | | | J = 1.8 Hz, 1H), 8.25 (d, J = |
| | (3-(dimethylamino)-2- | | | | 5.7 Hz, 1H), 7.52 (dd, J = |
| | oxopyrrolidin-1- | | | | 5.7, 1.9 Hz, 1H), 7.32-7.01 |
| | yl)pyridin-4-yl)-4,5- | | | | (m, 2H), 5.09 (d, J = 10.3 Hz, |
| | dimethyl-5- | | | | 1H), 4.26 (dd, J = 10.3, 7.7 |
| | (trifluoromethyl)tetrahydrofuran- | | | | Hz, 1H), 4.05-3.93 (m, 4H), |
| | 2-carboxamide | | | | 3.81-3.67 (m, 1H), 3.64 (t, |
| | (precursor was first | | | | J = 9.1 Hz, 1H), 2.78 (p, J = |
| | eluting isomer by SFC | | | | 7.5 Hz, 1H), 2.33 (s, 6H), |
| | using Whelk-O1 | | | | 2.22-2.06 (m, 1H), 1.96 (dq, |
| | column) | | | | J = 12.4, 9.1 Hz, 1H), 1.60 (s, |
| | | | | | 3H), 0.73 (d, J = 7.4 Hz, 3H) |
| | | | | | ppm. |
| 508 | rel-(2R*,3S*,4S*,5R*)- | 556.525 | 557.2 | 3.42 | ¹H NMR (500 MHz, DMSO- |
| | 3-(3,4-difluoro-2- | | | | d₆) δ 10.71 (s, 1H), 8.57 (d, |
| | methoxyphenyl)-N-(2- | | | | J = 1.9 Hz, 1H), 8.25 (d, J = |
| | (3-(dimethylamino)-2- | | | | 5.7 Hz, 1H), 7.50 (dd, J = |
| | oxopyrrolidin-1- | | | | 5.7, 2.0 Hz, 1H), 7.35-7.03 |
| | yl)pyridin-4-yl)-4,5- | | | | (m, 2H), 5.09 (d, J = 10.2 Hz, |
| | dimethyl-5- | | | | 1H), 4.25 (dd, J = 10.2, 7.7 |
| | (trifluoromethyl)tetrahydrofuran- | | | | Hz, 1H), 4.07-3.93 (m, 4H), |
| | 2-carboxamide | | | | 3.82-3.54 (m, 2H), 2.78 (p, |
| | (precursor was second | | | | J = 7.7 Hz, 1H), 2.33 (s, 6H), |
| | eluting isomer by SFC | | | | 2.23-2.05 (m, 1H), 1.96 (dq, |
| | using Whelk-O1 | | | | J = 12.6, 9.1 Hz, 1H), 1.60 (s, |
| | column) | | | | 3H), 0.73 (d, J = 7.3 Hz, 3H) |
| | | | | | ppm. |

The following compounds were made using a method similar to that described in Example 11, except that different amines were used in the Buchwald coupling step 2. Diastereomeric products of step 2 were separated by chiral SFC and Boc deprotection using General Method I, followed by N-methylation, using iodomethane and potassium carbonate in DMF, were used as the final steps on separated isomers. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 509 | rel-(2R*,3S*,4S*,5R*)- | 554.509 | 555.3 | 3.34 | ¹H NMR (500 MHz, DMSO- |
| | 3-(3,4-difluoro-2- | | | | d₆) δ 10.72 (s, 1H), 8.45 (s, |
| | methoxyphenyl)-4,5- | | | | 1H), 8.31 (d, J = 5.6 Hz, 1H), |
| | dimethyl-N-(2-((1S,5R)- | | | | 7.55 (dd, J = 5.7, 1.9 Hz, |
| | 6-methyl-2-oxo-3,6- | | | | 1H), 7.32-6.97 (m, 2H), |
| | diazabicyclo[3.1.1]heptan- | | | | 5.10 (d, J = 10.3 Hz, 1H), |
| | 3-yl)pyridin-4-yl)-5- | | | | 4.26 (dd, J = 10.3, 7.6 Hz, |
| | (trifluoromethyl)tetrahydrofuran- | | | | 1H), 4.09 (dd, J = 13.0, 2.5 |
| | 2-carboxamide | | | | Hz, 1H), 3.96 (d, J = 2.1 Hz, |
| | (precursor was first | | | | 3H), 3.89 (dd, J = 12.8, 1.0 |
| | eluting isomer by SFC | | | | Hz, 1H), 3.81 (d, J = 4.0 Hz, |
| | using Chiralpak IC | | | | 1H), 3.54 (dd, J = 5.8, 4.0 |
| | column) | | | | Hz, 1H), 2.78 (p, J = 7.6 Hz, |
| | | | | | 1H), 2.60-2.53 (partly obsc |
| | | | | | m, 1H), 2.14 (s, 3H), 1.75 (d, |
| | | | | | J = 8.6 Hz, 1H), 1.60 (s, 3H), |
| | | | | | 0.78-0.67 (m, 3H) ppm. |
| 510 | rel-(2R*,3S*,4S*,5R*)- | 554.509 | 555.3 | 3.34 | ¹H NMR (500 MHz, DMSO- |
| | 3-(3,4-difluoro-2- | | | | d₆) δ 10.71 (s, 1H), 8.43 (s, |
| | methoxyphenyl)-4,5- | | | | 1H), 8.31 (d, J = 5.6 Hz, 1H), |
| | dimethyl-N-(2-((1R,5S)- | | | | 7.55 (dd, J = 5.7, 1.8 Hz, |
| | 6-methyl-2-oxo-3,6- | | | | 1H), 7.25-7.02 (m, 2H), |
| | diazabicyclo[3.1.1]heptan- | | | | 5.10 (d, J = 10.3 Hz, 1H), |
| | 3-yl)pyridin-4-yl)-5- | | | | 4.25 (dd, J = 10.2, 7.6 Hz, |
| | (trifluoromethyl)tetrahydrofuran- | | | | 1H), 4.12 (dd, J = 13.0, 2.6 |
| | 2-carboxamide | | | | Hz, 1H), 3.96 (d, J = 2.1 Hz, |
| | (precursor was second | | | | 3H), 3.87-3.79 (m, 2H), |
| | eluting isomer by SFC | | | | 3.54 (dd, J = 5.7, 4.0 Hz, |
| | using Chiralpak IC | | | | 1H), 2.82-2.72 (m, 1H), |
| | column) | | | | 2.59-2.53 (partly obsc m, |
| | | | | | 1H), 2.14 (s, 3H), 1.76 (d, J = |
| | | | | | 8.6 Hz, 1H), 1.60 (s, 3H), |
| | | | | | 0.73 (d, J = 7.3 Hz, 3H) ppm. |

The following compounds were made using a method similar to that described in Example 11, except that different amines were used in the Buchwald coupling step 2 and General Method M was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 511 | (2R,3S,4S,5R)-3-(3,4- | 572.524 | 573 | 3.11 | ¹H NMR (500 MHz, DMSO- |
| | difluoro-2- | | | | d₆) δ 10.69 (s, 1H), 8.33 (d, J = |
| | methoxyphenyl)-N-(2- | | | | 5.6 Hz, 1H), 7.87 (d, J = |
| | ((R)-2-(hydroxymethyl)- | | | | 1.8 Hz, 1H), 7.54 (dd, J = |
| | 4-methyl-6- | | | | 5.7, 1.9 Hz, 1H), 7.29-7.06 |
| | oxopiperazin-1- | | | | (m, 2H), 5.10 (d, J = 10.3 Hz, |
| | yl)pyridin-4-yl)-4,5- | | | | 1H), 4.86 (t, J = 5.5 Hz, 1H), |
| | dimethyl-5- | | | | 4.44 (dd, J = 8.2, 3.9 Hz, |
| | (trifluoromethyl)tetrahydrofuran- | | | | 1H), 4.25 (dd, J = 10.3, 7.6 |
| | 2-carboxamide | | | | Hz, 1H), 3.96 (d, J = 2.0 Hz, |
| | | | | | 3H), 3.53-3.44 (m, 1H), |
| | | | | | 3.40 (dd, J = 16.8, 1.5 Hz, |
| | | | | | 1H), 3.25 (dt, J = 10.1, 4.0 |
| | | | | | Hz, 1H), 3.12-3.02 (m, 1H), |
| | | | | | 2.86 (d, J = 16.9 Hz, 1H), |
| | | | | | 2.77 (p, J = 7.5 Hz, 1H), 2.27 |
| | | | | | (s, 3H), 1.60 (s, 3H), 0.73 (d, |
| | | | | | J = 6.4 Hz, 3H) ppm; water |
| | | | | | peak obscures 1H. |
| 512 | (2R,3S,4S,5R)-3-(3,4- | 572.524 | 573 | 3.11 | ¹H NMR (500 MHz, DMSO- |
| | difluoro-2- | | | | d₆) δ 10.65 (s, 1H), 8.33 (d, J = |
| | methoxyphenyl)-N-(2- | | | | 5.6 Hz, 1H), 7.87 (d, J = |
| | ((S)-2-(hydroxymethyl)- | | | | 1.8 Hz, 1H), 7.53 (dd, J = |
| | 4-methyl-6- | | | | 5.7, 1.9 Hz, 1H), 7.21-7.04 |
| | oxopiperazin-1- | | | | (m, 2H), 5.09 (d, J = 10.3 Hz, |
| | yl)pyridin-4-yl)-4,5- | | | | 1H), 4.86 (t, J = 5.5 Hz, 1H), |
| | dimethyl-5- | | | | 4.41 (dd, J = 8.3, 3.9 Hz, |
| | (trifluoromethyl)tetrahydrofuran- | | | | 1H), 4.25 (dd, J = 10.3, 7.7 |
| | 2-carboxamide | | | | Hz, 1H), 3.96 (d, J = 2.1 Hz, |
| | | | | | 3H), 3.47 (td, J = 9.3, 8.9, 6.2 |
| | | | | | Hz, 1H), 3.43-3.36 (m, 1H), |
| | | | | | 3.28-3.18 (m, 1H), 3.13- |
| | | | | | 3.05 (m, 1H), 2.86 (d, J = |
| | | | | | 16.8 Hz, 1H), 2.77 (p, J = 7.5 |
| | | | | | Hz, 1H), 2.27 (s, 3H), 1.60 |
| | | | | | (s, 3H), 0.73 (d, J = 7.0 Hz, |
| | | | | | 3H) ppm; water peak |
| | | | | | obscures 1H. |

The following compound was made using a method similar to that described in Example 11, except that 6-bromopyrimidin-4-amine was used in step 1. The product of step 1 was treated with triethyl borane in heptanes/diethyl ether prior to the C—N coupling (step 2) which used potassium photophate in toluene at 110° C. instead of Cs₂CO₃ in dioxane. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 513 | (2R,3S,4S,5R)-3-(3,4- | 557.513 | 558.22 | 2.83 | 1H-NMR (400 MHz, |
| | difluoro-2- | | | | Chloroform-d) δ 8.94 (s, 1H), |
| | methoxyphenyl)-N-(6- | | | | 8.72 (dd, J = 5.3, 1.1 Hz, |
| | ((R)-2,4-dimethyl-6- | | | | 1H), 8.66 (s, 1H), 7.07-7.03 |
| | oxopiperazin-1- | | | | (m, 1H), 6.86 (td, J = 9.2, 7.6 |
| | yl)pyrimidin-4-yl)-4,5- | | | | Hz, 1H), 4.99 (d, J = 11.0 |
| | dimethyl-5- | | | | Hz, 1H), 4.07 (dd, J = 11.0, |
| | (trifluoromethyl)tetrahydrofuran- | | | | 7.8 Hz, 1H), 4.00 (d, J = 2.7 |
| | 2-carboxamide | | | | Hz, 3H), 3.62 (s, 1H), 3.14 |
| | | | | | (s, 2H), 2.80 (d, J = 15.1 Hz, |
| | | | | | 2H), 2.78-2.70 (m, 1H), 2.46 |
| | | | | | (s, 3H), 1.67 (m, 3H), 1.32- |
| | | | | | 1.28 (m, 3H), 0.77-0.75 (m, |
| | | | | | 3H) ppm. |

The following compound was made using a method similar to that described in Example 11, except that (2R,3S,4S,5R)-3-(2-(difluoromethoxy)-3,4-difluorophenyl)-4,5-dimethyl-5-(trifluoromethyl)tetrahydrofuran-2-carboxylic acid was used in step 1 and tert-butyl 3-oxopiperazine-1-carboxylate was used in the Buchwald coupling step 2. Deprotection using General Method I was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 514 | (2R,3S,4S,5R)-3-(2- | 564.453 | 565 | 3.11 | ¹H NMR (500 MHz, DMSO- |
| | (difluoromethoxy)- | | | | d₆) δ 10.65 (s, 1H), 8.30 (d, J = |
| | 3,4-difluorophenyl)- | | | | 5.6 Hz, 1H), 8.10 (d, J = |
| | 4,5-dimethyl-N-(2- | | | | 1.8 Hz, 1H), 7.65-7.01 (m, |
| | (2-oxopiperazin-1- | | | | 4H), 5.14 (d, J = 10.2 Hz, |
| | yl)pyridin-4-yl)-5- | | | | 1H), 4.28 (dd, J = 10.2, 7.7 |
| | (trifluoromethyl)tetrahydrofuran- | | | | Hz, 1H), 3.78 (tt, J = 12.2, |
| | 2-carboxamide | | | | 6.1 Hz, 2H), 3.43 (s, 2H), |
| | | | | | 3.00 (t, J = 5.5 Hz, 2H), 2.79- |
| | | | | | 2.73 (m, 2H), 1.59 (s, 3H), |
| | | | | | 0.76 (d, J = 7.4 Hz, 3H) ppm. |

The following compound was made using a method similar to that described in Example 11, except that (2R,3S,4S,5R)-3-(3,4-difluoro-2-(methoxy-d₃)phenyl)-4,5-dimethyl-5-(trifluoromethyl)tetrahydrofuran-2-carboxylic acid was used in step 1 and tert-butyl 3-oxopiperazine-1-carboxylate was used in the Buchwald coupling step 2. Deprotection using General Method I was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 515 | (2R,3S,4S,5R)-3-(3,4- | 531.49 | 532 | 3.06 | ¹H NMR (500 MHz, DMSO- |
| | difluoro-2-(methoxy- | | | | d₆) δ 10.66 (s, 1H), 8.30 (d, J = |
| | d₃)phenyl)-4,5- | | | | 5.6 Hz, 1H), 8.10 (d, J = |
| | dimethyl-N-(2-(2- | | | | 1.9 Hz, 1H), 7.52 (dd, J = |
| | oxopiperazin-1- | | | | 5.6, 1.9 Hz, 1H), 7.26-7.00 |
| | yl)pyridin-4-yl)-5- | | | | (m, 2H), 5.09 (d, J = 10.2 Hz, |
| | (trifluoromethyl)tetrahydrofuran- | | | | 1H), 4.25 (dd, J = 10.2, 7.6 |
| | 2-carboxamide | | | | Hz, 1H), 3.99-3.63 (m, 2H), |
| | | | | | 3.43 (s, 2H), 3.00 (t, J = 5.5 |
| | | | | | Hz, 2H), 2.80-2.66 (m, 2H), |
| | | | | | 1.60 (s, 3H), 0.73 (d, J = 6.9 |
| | | | | | Hz, 3H) ppm. |

The following compounds were made using a method similar to that described in Example 11, except that (2R,3S,4S,5R)-3-(2-(difluoromethoxy)-3,4-difluorophenyl)-4,5-dimethyl-5-(trifluoromethyl)tetrahydrofuran-2-carboxylic acid was used in step 1 and 4-methylpiperazin-2-one was used in the C-N coupling, step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 516 | (2R,3S,4S,5R)-3-(2- | 578.479 | 579 | 3.27 | ¹H NMR (500 MHz, DMSO- |
| | (difluoromethoxy)-3,4- | | | | d₆) δ 10.67 (s, 1H), 8.30 (d, J = |
| | difluorophenyl)-4,5- | | | | 5.6 Hz, 1H), 8.08 (s, 1H), 7.65- |
| | dimethyl-N-(2-(4- | | | | 7.05 (m, 4H), 5.13 (d, J = |
| | methyl-2- | | | | 10.1 Hz, 1H), 4.28 (dd, J = |
| | oxopiperazin-1- | | | | 10.2, 7.6 Hz, 1H), 3.83 (hept, J = |
| | yl)pyridin-4-yl)-5- | | | | 6.8, 6.2 Hz, 2H), 3.16 (s, |
| | (trifluoromethyl)tetrahydrofuran- | | | | 2H), 2.80-2.68 (m, 3H), 2.28 |
| | 2-carboxamide | | | | (s, 3H), 1.58 (s, 3H), 0.76 (d, J = |
| | | | | | 7.4 Hz, 3H) ppm. |

The following compounds were made using a method similar to that described in Example 11, except that (2R,3S,4S,5R)-3-(2-(difluoromethoxy)-3,4-difluorophenyl)-4,5-dimethyl-5-(trifluoromethyl)tetrahydrofuran-2-carboxylic acid was used in step 1 and (6R)-4,6-dimethylpiperazin-2-one was used in the C-N coupling, step 2 with heating at 130° C. for 90 minutes. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 517 | (2R,3S,4S,5R)-3-(2- | 592.506 | 593 | 3.36 | ¹H NMR (500 MHz, DMSO- |
| | (difluoromethoxy)-3,4- | | | | d₆) δ 10.62 (s, 1H), 8.35 (d, J = |
| | difluorophenyl)-N-(2- | | | | 5.7 Hz, 1H), 7.85 (d, J = |
| | ((R)-2,4-dimethyl-6- | | | | 1.6 Hz, 1H), 7.55-7.45 (m, |
| | oxopiperazin-1- | | | | 2H), 7.33-7.30 (m, 1H), |
| | yl)pyridin-4-yl)-4,5- | | | | 7.21 (t, J = 72.4 Hz, 1H), |
| | dimethyl-5- | | | | 5.14 (d, J = 10.2 Hz, 1H), |
| | (trifluoromethyl)tetrahydrofuran- | | | | 4.53 (q, J = 4.9 Hz, 1H), 4.29 |
| | 2-carboxamide | | | | (dd, J = 10.3, 7.7 Hz, 1H), |
| | | | | | 2.94 (d, J = 16.7 Hz, 1H), |
| | | | | | 2.81-2.68 (m, 2H), 2.66- |
| | | | | | 2.58 (m, 1H), 2.27 (s, 3H), |
| | | | | | 1.59 (s, 3H), 1.05 (d, J = 6.3 |
| | | | | | Hz, 3H), 0.76 (d, J = 7.4 Hz, |
| | | | | | 3H) ppm; water peak |
| | | | | | obscures 1H. |

The following compound was made using a method similar to that described in Example 11, except that (2R,3S,4S,5R)-3-(3,4-difluoro-2-(methoxy-d₃)phenyl)-4,5-dimethyl-5-(trifluoromethyl)tetrahydrofuran-2-carboxylic acid was used in step 1 and 4-methylpiperazin-2-one was used in the C-N coupling, step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 518 | (2R,3S,4S,5R)-3-(3,4- | 545.517 | 546.2 | 3.23 | ¹H NMR (500 MHz, DMSO- |
| | difluoro-2-(methoxy- | | | | d₆) δ 10.67 (s, 1H), 8.31 (d, J = |
| | d3)phenyl)-4,5- | | | | 5.7 Hz, 1H), 8.10 (d, J = |
| | dimethyl-N-(2-(4- | | | | 1.9 Hz, 1H), 7.53 (dd, J = |
| | methyl-2-oxopiperazin- | | | | 5.6, 1.9 Hz, 1H), 7.30-7.02 |
| | 1-yl)pyridin-4-yl)-5- | | | | (m, 2H), 5.09 (d, J = 10.3 Hz, |
| | (trifluoromethyl)tetrahydrofuran- | | | | 1H), 4.25 (dd, J = 10.3, 7.7 |
| | 2-carboxamide | | | | Hz, 1H), 3.83 (h, J = 6.8 Hz, |
| | | | | | 2H), 3.17 (s, 2H), 2.83-2.66 |
| | | | | | (m, 3H), 2.28 (s, 3H), 1.60 |
| | | | | | (s, 3H), 0.73 (d, J = 6.9 Hz, |
| | | | | | 3H) ppm. |

The following compound was made using a method similar to that described in Example 11, except that (2R,3S,4S,5R)-3-(3,4-difluoro-2-(methoxy-d₃)phenyl)-4,5-dimethyl-5-(trifluoromethyl)tetrahydrofuran-2-carboxylic acid was used in step 1 and (6R)-4,6-dimethylpiperazin-2-one was used in the C-N coupling, step 2 with heating at 130° C. for 105 mins. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 519 | (2R,3S,4S,5R)-3-(3,4- | 559.543 | 560 | 3.32 | ¹H NMR (500 MHz, DMSO- |
| | difluoro-2-(methoxy- | | | | d₆) δ 10.63 (s, 1H), 8.35 (d, J = |
| | d₃)phenyl)-N-(2-((R)- | | | | 5.6 Hz, 1H), 7.86 (s, 1H), 7.52 |
| | 2,4-dimethyl-6- | | | | (dd, J = 5.5, 1.9 Hz, 1H), 7.27- |
| | oxopiperazin-1- | | | | 7.06 (m, 2H), 5.10 (d, J = |
| | yl)pyridin-4-yl)-4,5- | | | | 10.3 Hz, 1H), 4.54 (q, J = 5.0 |
| | dimethyl-5- | | | | Hz, 1H), 4.25 (dd, J = 10.3, 7.6 |
| | (trifluoromethyl)tetrahydrofuran- | | | | Hz, 1H), 2.94 (d, J = 16.7 Hz, |
| | 2-carboxamide | | | | 1H), 2.77 (p, J = 7.5 Hz, 1H), |
| | | | | | 2.71 (dd, J = 11.8, 4.2 Hz, 1H), |
| | | | | | 2.66-2.58 (m, 1H), 2.27 (s, |
| | | | | | 3H), 1.60 (s, 3H), 1.05 (d, J = |
| | | | | | 6.4 Hz, 3H), 0.73 (d, J = 7.3 |
| | | | | | Hz, 3H) ppm; water peak |
| | | | | | obscures 1H. |

The following compounds were made using a method similar to that described in Example 11, except that an S_{N}Ar reaction was performed in step 2 (rather than palladium catalysed C-N coupling) by heating the product of step 1 with different amines in 2-methyltetrahydrofuran at 100° C. for 20 hours. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 520 | (2R,3S,4S,5R)-3-(3,4- | 529.5 | 530 | 3.47 | ¹H NMR (500 MHz, DMSO- |
| | difluoro-2- | | | | d₆) δ 10.31 (s, 1H), 7.92 (d, J = |
| | methoxyphenyl)-N-(2- | | | | 5.6 Hz, 1H), 7.18 (td, J = 9.5, |
| | ((R)-2- | | | | 7.6 Hz, 1H), 7.11 (td, J = 7.5, |
| | (hydroxymethyl)pyrrol | | | | 6.6, 3.1 Hz, 1H), 6.81 (d, J = |
| | idin-1-yl)pyridin-4- | | | | 1.7 Hz, 1H), 6.79 (dd, J = 5.6, |
| | yl)-4,5-dimethyl-5- | | | | 1.7 Hz, 1H), 5.06 (d, J = 10.3 |
| | (trifluoromethyl)tetrahydrofuran- | | | | Hz, 1H), 4.93 (t, J = 5.4 Hz, |
| | 2-carboxamide | | | | 1H), 4.23 (dd, J = 10.4, 7.6 Hz, |
| | | | | | 1H), 3.99 (d, J = 7.4 Hz, 1H), |
| | | | | | 3.96 (d, J = 2.1 Hz, 3H), 3.55 |
| | | | | | (dt, J = 9.8, 4.7 Hz, 1H), 3.40- |
| | | | | | 3.33 (m, 1H), 3.27-3.21 (m, |
| | | | | | 1H), 3.17 (d, J = 7.0 Hz, 1H), |
| | | | | | 2.77 (p, J = 7.5 Hz, 1H), 1.97 |
| | | | | | (d, J = 8.8 Hz, 2H), 1.92-1.80 |
| | | | | | (m, 2H), 1.59 (s, 3H), 0.75- |
| | | | | | 0.70 (m, 3H) ppm. |
| 521 | (2R,3S,4S,5R)-3-(3,4- | 503.462 | 504 | 3.29 | ¹H NMR (500 MHz, DMSO- |
| | difluoro-2- | | | | d₆) δ 10.28 (s, 1H), 7.92 (d, J = |
| | methoxyphenyl)-N-(2- | | | | 5.6 Hz, 1H), 7.18 (td, J = 9.8, |
| | ((2-hydroxyethyl)(methyl) | | | | 9.4, 7.6 Hz, 1H), 7.11 (td, J = |
| | amino)pyridin-4-yl)- | | | | 8.1, 7.0, 3.5 Hz, 1H), 6.93 (d, J = |
| | 4,5-dimethyl-5- | | | | 1.6 Hz, 1H), 6.76 (dd, J = |
| | (trifluoromethyl)tetrahydrofuran- | | | | 5.6, 1.7 Hz, 1H), 5.06 (d, J = |
| | 2-carboxamide | | | | 10.3 Hz, 1H), 4.70-4.63 (m, |
| | | | | | 1H), 4.23 (dd, J = 10.4, 7.6 Hz, |
| | | | | | 1H), 3.96 (d, J = 2.1 Hz, 3H), |
| | | | | | 3.53 (d, J = 2.9 Hz, 3H), 2.98 |
| | | | | | (s, 2H), 2.77 (p, J = 7.5 Hz, |
| | | | | | 1H), 2.55 (s, 2H), 1.59 (s, 3H), |
| | | | | | 0.73 (dd, J = 7.1, 2.4 Hz, 3H) |
| | | | | | ppm. |

The following compounds were made using a method similar to that described in Example 11, except that alternative conditions were used in step 2. The product of step 1 was stirred with 2-(dimethylamino)-N-methyl-acetamide (1.05 equiv), potassium carbonate (1.03 equiv), copper iodide (5 mol % o) and (1R,2R)—N1,N2-dimethylcyclohexane-1,2-diamine (14 mol % o) in dioxane under argon at 120° C. for 2 hours. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 522 | (2R,3S,4S,5R)-3-(3,4- | 544.514 | 545.21 | 2.52 | ¹H-NMR (400 MHz, |
| | difluoro-2- | | | | Chloroform-d) δ 8.51 (s, 1H), |
| | methoxyphenyl)-N-(2- | | | | 8.33 (d, J = 5.5 Hz, 1H), 7.62 |
| | (2-(dimethylamino)-N- | | | | (d, J = 1.8 Hz, 1H), 7.40 (d, J = |
| | methylacetamido)pyridin- | | | | 5.0 Hz, 1H), 7.08-7.05 (m, |
| | 4-yl)-4,5-dimethyl-5- | | | | 1H), 6.93-6.87 (m, 1H), 5.01 |
| | (trifluoromethyl)tetrahydrofuran- | | | | (d, J = 11.0 Hz, 1H), 4.08 (dd, |
| | 2-carboxamide | | | | J = 11.0, 8.2 Hz, 1H), 4.00 (d, |
| | | | | | J = 2.7 Hz, 3H), 3.37 (s, 3H), |
| | | | | | 3.26 (s, 2H), 2.75 (t, J = 7.8 |
| | | | | | Hz, 1H), 2.28 (s, 6H), 1.68 (s, |
| | | | | | 3H), 0.79-0.77 (m, 3H) |

### Example 12

(2R,3S,4S,5R)—N-(6-aminopyridin-3-yl)-3-(3,4-difluoro-2-methoxyphenyl)-4,5-dimethyl-5-(trifluoromethyl)tetrahydrofuran-2-carboxamide (523)

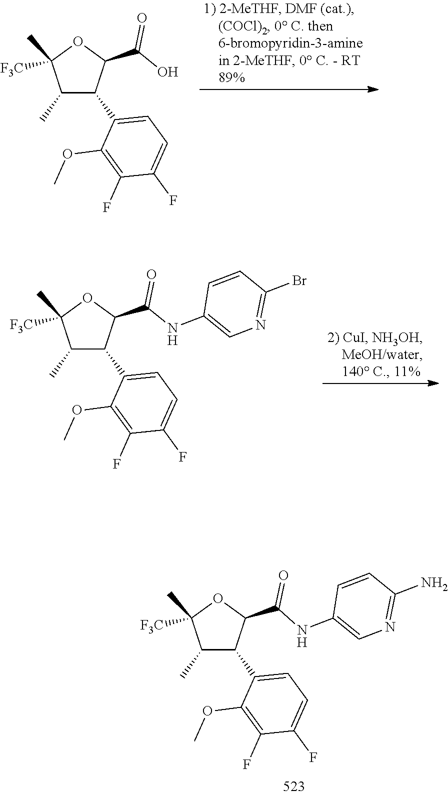 [SMILES: COc1c([C@H]2[C@H](C(=O)O)O[C@@](C)(C(F)(F)F)[C@H]2C)ccc(F)c1F ; COc1c([C@H]2[C@H](C(=O)Nc3ccc(Br)nc3)O[C@@](C)(C(F)(F)F)[C@H]2C)ccc(F)c1F ; COc1c([C@H]2[C@H](C(=O)Nc3ccc(N)nc3)O[C@@](C)(C(F)(F)F)[C@H]2C)ccc(F)c1F]

### Step 1:

(2R,3S,4S,5R)-3-(3,4-difluoro-2-methoxyphenyl)-4,5-dimethyl-5-(trifluoromethyl)tetrahydrofuran-2-carboxylic acid (2.17 g, 6.125 mmol) was dissolved in 2-MeTHF (15 mL) under nitrogen and the solution cooled in an ice bath. DMF (90 μL, 1.162 mmol) was added followed by the slow addition of (COCl)₂ (1.3 mL, 14.90 mmol). After 5 minutes the cooling bath was removed. After 1 hour the reaction mixture was concentrated in vacuo and the residue taken up in 2-MeTHF (15 mL) under nitrogen. This solution was added to a stirred solution of 6-bromopyridin-3-amine (1.80 g, 10.40 mmol) and DIPEA (3.2 mL, 18.37 mmol) i n 2-MeTHF (15 mL) with cooling in an ice bath. The cooling bath was removed and reaction mixture stirred for 1 hour, then partitioned between EtOAc and water. The aqueous phase was extracted with EtOAc and the combined organics were washed with brine, dried (Na₂SO₄), filtered, and concentrated in vacuo. The residue was purified by flash chromatography (SiO₂, 0 to 100% EtOAc in heptanes) to give (2R,3S,4S,5R)—N-(6-bromopyridin-3-yl)-3-(3,4-difluoro-2-methoxyphenyl)-4,5-dimethyl-5-(trifluoromethyl)tetrahydrofuran-2-carboxamide (2.09 g, 67%) as a white solid. ¹H NMR (500 MHz, DMSO-d₆) δ 10.51 (s, 1H), 8.63 (d, J=2.7 Hz, 1H), 8.01 (dd, J=8.7, 2.8 Hz, 1H), 7.60 (d, J=8.7 Hz, 1H), 7.44-7.06 (m, 2H), 5.10 (d, J=10.2 Hz, 1H), 4.25 (dd, J=10.3, 7.7 Hz, 1H), 3.95 (d, J=2.0 Hz, 3H), 2.77 (p, J=7.5 Hz, 1H), 1.61 (s, 3H), 0.73 (dd, J=7.6, 2.3 Hz, 3H) ppm. ESI-MS m/z calc. 508.04208, found 507.0 (M−1)⁻; Retention time: 1.06 minutes.

### Step 2:

(2R,3S,4S,5R)—N-(6-bromopyridin-3-yl)-3-(3,4-difluoro-2-methoxyphenyl)-4,5-dimethyl-5-(trifluoromethyl)tetrahydrofuran-2-carboxamide (50 mg, 0.09818 mmol) was combined with aqueous ammonia hydroxide (250 μL of 35% w/v, 2.497 mmol) and CuI (2 mg, 0.01050 mmol) in a reaction tube. MeOH (1 mL) was added and the mixture heated at 140° C. μW for 1 hour. The reaction mixture was concentrated and the residue taken up in DMSO/MeOH, filtered and purified by preparative reversed-phase HPLC (basic elutent) to give (2R,3S,4S,5R)—N-(6-aminopyridin-3-yl)-3-(3,4-difluoro-2-methoxyphenyl)-4,5-dimethyl-5-(trifluoromethyl)tetrahydrofuran-2-carboxamide (523, 4.89 mg, 11%) as a pale yellow solid. ¹H NMR (500 MHz, DMSO-d₆) δ 9.90 (s, 1H), 8.07 (d, J=2.6 Hz, 1H), 7.53 (dd, J=8.8, 2.7 Hz, 1H), 7.33-7.07 (m, 2H), 6.39 (d, J=8.9 Hz, 1H), 5.78 (s, 2H), 5.01 (d, J=10.5 Hz, 1H), 4.21 (dd, J=10.5, 7.6 Hz, 1H), 3.95 (d, J=2.0 Hz, 3H), 2.74 (p, J=7.4 Hz, 1H), 1.59 (s, 3H), 0.72 (d, J=7.3 Hz, 3H) ppm. ESI-MS m/z calc. 445.1425, found 446.0 (M+1); 444.0 (M−1)⁻; Retention time: 3.15 minutes.

The following compound was made using a method similar to that described in Example 12, except that methylamine was used in step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 524 | (2R,3S,4S,5R)-3-(3,4- | 459.41 | 460 | 3.33 | ¹H NMR (500 MHz, DMSO-d₆) |
| | difluoro-2- | | | | δ 9.92 (s, 1H), 8.13 (d, J = 2.6 |
| | methoxyphenyl)-4,5- | | | | Hz, 1H), 7.56 (dd, J = 8.9, 2.6 |
| | dimethyl-N-(6- | | | | Hz, 1H), 7.29-7.06 (m, 2H), |
| | (methylamino)pyridin- | | | | 6.40-6.34 (m, 2H), 5.01 (d, J = |
| | 3-yl)-5- | | | | 10.5 Hz, 1H), 4.21 (dd, J = 10.4, |
| | (trifluoromethyl)tetrahydrofuran- | | | | 7.6 Hz, 1H), 3.95 (d, J = 1.9 Hz, |
| | 2-carboxamide | | | | 3H), 2.81-2.67 (m, 4H), 1.59 |
| | | | | | (s, 3H), 0.81-0.62 (m, 3H) |
| | | | | | ppm. |

The following compounds were made using a method similar to that described in Example 12, except that C-N coupling conditions similar to those previously described (Example 11, step 2) were used in step 2 with acetamide and methanesulfonamide respectively as coupling partners. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 525 | (2R,3S,4S,5R)-N-(6- | 487.42 | 488 | 3.25 | ¹H NMR (500 MHz, DMSO- |
| | acetamidopyridin-3-yl)- | | | | d₆) δ 10.44 (s, 1H), 10.30 (s, |
| | 3-(3,4-difluoro-2- | | | | 1H), 8.55 (d, J = 2.6 Hz, 1H), |
| | methoxyphenyl)-4,5- | | | | 8.02 (d, J = 9.0 Hz, 1H), 7.93 |
| | dimethyl-5- | | | | (dd, J = 9.0, 2.7 Hz, 1H), |
| | (trifluoromethyl)tetrahydrofuran- | | | | 7.17 (dd, J = 9.2, 6.3 Hz, |
| | 2-carboxamide | | | | 2H), 5.08 (d, J = 10.4 Hz, |
| | | | | | 1H), 4.24 (dd, J = 10.4, 7.6 |
| | | | | | Hz, 1H), 3.96 (d, J = 2.0 Hz, |
| | | | | | 3H), 2.76 (p, J = 7.6 Hz, 1H), |
| | | | | | 2.06 (s, 3H), 1.61 (s, 3H), |
| | | | | | 0.73 (dd, J = 7.5, 2.4 Hz, 3H) |
| | | | | | ppm. |
| 526 | (2R,3S,4S,5R)-3-(3,4- | 523.473 | 524 | 3.10 | ¹H NMR (500 MHz, DMSO- |
| | difluoro-2- | | | | d₆) δ 10.47 (s, 1H), 10.28 (s, |
| | methoxyphenyl)-4,5- | | | | 1H), 8.53-8.29 (m, 1H), |
| | dimethyl-N-(6- | | | | 7.92 (dd, J = 9.0, 2.5 Hz, |
| | (methylsulfonamido)pyridin- | | | | 1H), 7.28-7.07 (m, 2H), |
| | 3-yl)-5- | | | | 6.94 (d, J = 8.8 Hz, 1H), 5.07 |
| | (trifluoromethyl)tetrahydrofuran- | | | | (d, J = 10.3 Hz, 1H), 4.23 |
| | 2-carboxamide | | | | (dd, J = 10.3, 7.7 Hz, 1H), |
| | | | | | 3.95 (d, J = 2.0 Hz, 3H), 3.22 |
| | | | | | (s, 3H), 2.76 (p, J = 7.4 Hz, |
| | | | | | 1H), 1.60 (s, 3H), 0.73 (d, J = |
| | | | | | 7.2 Hz, 3H) ppm. |

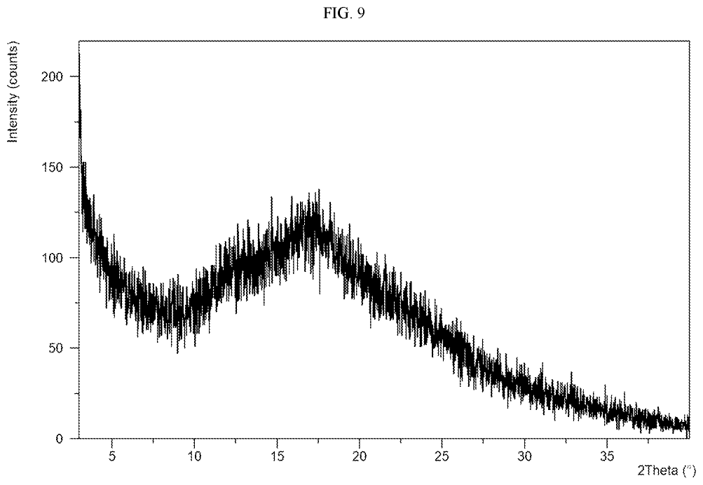

Compound 525 was analyzed by X-ray powder diffraction and determined to be amorphous (see FIG. 9).

### Example 13

rel-(2S,3R,4R,5S)-3-(3,4-difluoro-2-methoxyphenyl)-N-(2-(N,S-dimethylsulfonimidoyl)pyridin-4-yl)-4,5-dimethyl-5-(trifluoromethyl)tetrahydrofuran-2-carboxamide (527) and rel-(2R,3S,4S,5R)-3-(3,4-difluoro-2-methoxyphenyl)-N-(2-(N,S-dimethylsulfonimidoyl)pyridin-4-yl)-4,5-dimethyl-5-(trifluoromethyl)tetrahydrofuran-2-carboxamide (528)

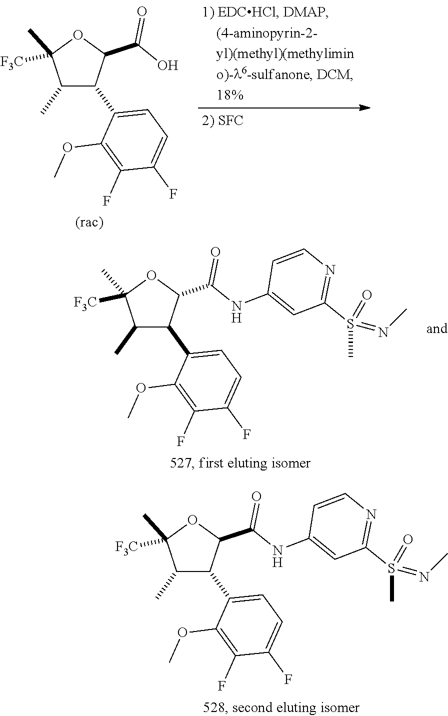 [SMILES: COc1c([C@H]2[C@H](C(=O)O)O[C@@](C)(C(F)(F)F)[C@H]2C)ccc(F)c1F ; CN=[S@](C)(=O)c1cc(NC(=O)[C@H]2O[C@](C)(C(F)(F)F)[C@H](C)[C@@H]2c2ccc(F)c(F)c2OC)ccn1 ; CN=[S@@](C)(=O)c1cc(NC(=O)[C@@H]2O[C@@](C)(C(F)(F)F)[C@@H](C)[C@H]2c2ccc(F)c(F)c2OC)ccn1]

### Step 1:

To a solution of 2-(N,S-dimethylsulfonimidoyl)pyridin-4-amine (33 mg, 0.18 mmol, second eluting peak by SFC using a Chiralcel OJ column) and rac-(2R,3S,4S,5R)-3-(3,4-difluoro-2-methoxyphenyl)-4,5-dimethyl-5-(trifluoromethyl)tetrahydrofuran-2-carboxylic acid (64 mg, 0.16 mmol) in DCM (1.5 mL) was added DMAP (43 mg, 0.35 mmol) followed by EDC hydrochloride (67.6 mg, 0.35 mmol). The reaction mixture was stirred at ambient temperature for 20 hours before additional EDC hydrochloride was added. The reaction was stirred for a further 20 hours, then diluted with DCM and washed with water. The aqueous layer was extracted with DCM and the combined organic layers were dried and concentrated in vacuo. Purification by flash column chromatography (4 g SiO₂, 0 to 100% EtOAc in heptane) gave rac-(2R,3S,4S,5R)-3-(3,4-difluoro-2-methoxyphenyl)-N-(2-(N,S-dimethylsulfonimidoyl)pyridin-4-yl)-4,5-dimethyl-5-(trifluoromethyl)tetrahydrofuran-2-carboxamide (17 mg, 18%).

### Step 2:

rac-(2R,3S,4S,5R)-3-(3,4-difluoro-2-methoxyphenyl)-N-(2-(N,S-dimethylsulfonimidoyl)pyridin-4-yl)-4,5-dimethyl-5-(trifluoromethyl)tetrahydrofuran-2-carboxamide (17 mg, 18%) was purified by chiral SFC using a Chiralpak AS-H column, 5 μm particle size, 25 cm×10 mm from Daicel on a Minigram SFC instrument from Berger Instruments to give:

First eluting isomer (rt=2.26 mins): rel-(2S,3R,4R,5S)-3-(3,4-difluoro-2-methoxyphenyl)-N-(2-(N,S-dimethylsulfonimidoyl)pyridin-4-yl)-4,5-dimethyl-5-(trifluoromethyl)tetrahydrofuran-2-carboxamide (527, 4.4 mg). ¹H NMR (500 MHz, Methanol-d₄) δ 8.62 (d, J=5.4 Hz, 1H), 8.43 (d, J=1.9 Hz, 1H), 7.92 (dd, J=5.5, 2.1 Hz, 1H), 7.14 (ddd, J=8.1, 5.6, 2.2 Hz, 1H), 7.00 (ddd, J=10.0, 8.9, 7.5 Hz, 1H), 5.12 (d, J=10.4 Hz, 1H), 4.35 (dd, J=10.4, 8.0 Hz, 1H), 4.02 (d, J=2.3 Hz, 3H), 3.23 (s, 3H), 2.82 (p, J=7.6 Hz, 1H), 2.59 (s, 3H), 1.68 (s, 3H), 0.84 (dq, J=7.4, 2.4 Hz, 3H) ppm; ESI-MS m/z calc. 521.14075, found 522.6 (M+1)⁺; 520.8 (M−1)⁻.

Second eluting isomer (rt=3.70 mins): rel-(2R,3S,4S,5R)-3-(3,4-difluoro-2-methoxyphenyl)-N-(2-(N,S-dimethylsulfonimidoyl)pyridin-4-yl)-4,5-dimethyl-5-(trifluoromethyl)tetrahydrofuran-2-carboxamide (528, 5.4 mg). ¹H NMR (500 MHz, Methanol-d₄) δ 8.62 (dd, J=5.5, 0.6 Hz, 1H), 8.43 (dd, J=2.1, 0.6 Hz, 1H), 7.92 (dd, J=5.5, 2.1 Hz, 1H), 7.14 (ddd, J=8.1, 5.6, 2.1 Hz, 1H), 7.05-6.96 (m, 1H), 5.12 (d, J=10.4 Hz, 1H), 4.35 (dd, J=10.4, 8.1 Hz, 1H), 4.02 (d, J=2.3 Hz, 3H), 3.23 (s, 3H), 2.82 (p, J=7.6 Hz, 1H), 2.59 (s, 3H), 1.68 (d, J=1.1 Hz, 3H), 0.84 (dt, J=7.4, 2.4 Hz, 3H) ppm; ESI-MS m/z calc. 521.14075, found 522.1 (M+1)⁺; 520.1 (M−1)⁻.

The following compounds were made using a method similar to that described in Example 13, except that 2-(N,S-dimethylsulfonimidoyl)pyridin-4-amine (first eluting peak by SFC using a Chiralcel OJ column) was used as the coupling partner in step 1. After treating with EDC in step 1, as described in Example 13, DIPEA and T3P were added and the mixture was stirred for an additional 68 hours at ambient temperature. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 529 | rel-(2S,3R,4R,5S)-3- | 521.501 | 522.1 | 3.18 | ¹H NMR (500 MHz, DMSO- |
| | (3,4-difluoro-2- | | | | d₆) δ 10.89 (s, 1H), 8.61 (d, J = |
| | methoxyphenyl)-N-(2- | | | | 5.4 Hz, 1H), 8.33 (d, J = |
| | (N,S- | | | | 2.0 Hz, 1H), 7.87-7.82 (m, |
| | dimethylsulfonimidoyl) | | | | 1H), 7.21-7.14 (m, 2H), |
| | pyridin-4-yl)-4,5- | | | | 5.12 (d, J = 10.1 Hz, 1H), |
| | dimethyl-5- | | | | 4.27 (dd, J = 10.2, 7.7 Hz, |
| | (trifluoromethyl)tetrahydrofuran- | | | | 1H), 3.96 (d, J = 2.1 Hz, 3H), |
| | 2-carboxamide | | | | 3.16 (s, 3H), 2.79 (q, J = 7.6 |
| | (first eluting isomer by | | | | Hz, 1H), 2.46 (s, 3H), 1.62 |
| | SFC using Chiralpak | | | | (s, 3H), 1.46 (s, 0H), 0.76- |
| | AS-H column) | | | | 0.71 (m, 3H) ppm. |
| 530 | rel-(2R,3S,4S,5R)-3- | 521.501 | 522.1 | 3.18 | ¹H NMR (500 MHz, DMSO- |
| | (3,4-difluoro-2- | | | | d₆) δ 10.88 (s, 1H), 8.62 (d, J = |
| | methoxyphenyl)-N-(2- | | | | 5.5 Hz, 1H), 8.35 (d, J = |
| | (N,S- | | | | 2.0 Hz, 1H), 7.84 (dd, J = |
| | dimethylsulfonimidoyl) | | | | 5.5, 2.0 Hz, 1H), 7.21-7.14 |
| | pyridin-4-yl)-4,5- | | | | (m, 2H), 5.13 (d, J = 10.2 Hz, |
| | dimethyl-5- | | | | 1H), 4.27 (dd, J = 10.2, 7.7 |
| | (trifluoromethyl)tetrahydrofuran- | | | | Hz, 1H), 3.96 (d, J = 2.2 Hz, |
| | 2-carboxamide | | | | 3H), 3.16 (s, 3H), 2.78 (p, J = |
| | (second eluting isomer | | | | 7.5 Hz, 1H), 2.46 (s, 3H), |
| | by SFC using Chiralpak | | | | 1.62 (s, 3H), 0.74 (d, J = 7.0 |
| | AS-H column) | | | | Hz, 3H) ppm. |

### Example 14

rel-(2S,3R,4R,5S)-3-(3,4-difluoro-2-methoxyphenyl)-N-(2-(1-hydroxyethyl)pyridin-4-yl)-4,5-dimethyl-5-(trifluoromethyl)tetrahydrofuran-2-carboxamide (531) and rel-(2R,3S,4S,5R)-3-(3,4-difluoro-2-methoxyphenyl)-N-(2-(1-hydroxyethyl)pyridin-4-yl)-4,5-dimethyl-5-(trifluoromethyl)tetrahydrofuran-2-carboxamide (532)

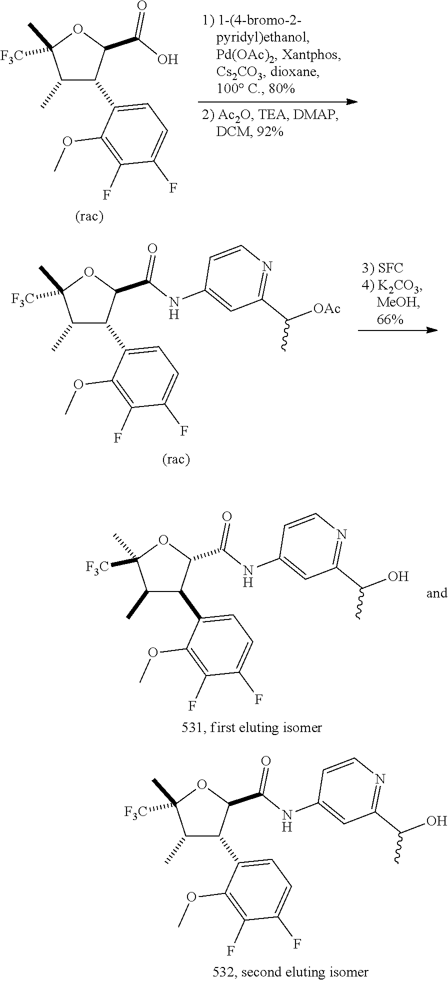

### Step 1:

To a solution of rac-(2R,3S,4S,5R)-3-(3,4-difluoro-2-methoxyphenyl)-4,5-dimethyl-5-(trifluoromethyl)tetrahydrofuran-2-carboxamide (50 mg, 0.14 mmol) in dioxane (4 mL) was added 1-(4-bromo-2-pyridyl)ethanol (43.8 mg, 0.22 mmol, first eluting isomer by SFC using Chiralpak ID column), Pd(OAc)₂ (11 mg, 0.049 mmol), Xantphos (44.8 mg, 0.077 mmol) and cesium carbonate (120 mg, 0.37 mmol) and the mixture was stirred at 100° C. under nitrogen for 3 hours. The reaction mixture was cooled to ambient temperature and partitioned between EtOAc and brine, and the layers separated. The aqueous layer was extracted with EtOAc (×2) and the combined organic extracts dried (MgSO₄), filtered, and concentrated in vacuo to give rac-(2R,3S,4S,5R)-3-(3,4-difluoro-2-methoxyphenyl)-N-(2-(1-hydroxyethyl)pyridin-4-yl)-4,5-dimethyl-5-(trifluoromethyl)tetrahydrofuran-2-carboxamide (53 mg, 79%). ESI-MS m/z calc. 474.1578, found 475.2 (M+1)⁺; 473.3 (M−1)⁻.

### Step 2:

To a solution of rac-(2R,3S,4S,5R)-3-(3,4-difluoro-2-methoxyphenyl)-N-(2-(1-hydroxyethyl)pyridin-4-yl)-4,5-dimethyl-5-(trifluoromethyl)tetrahydrofuran-2-carboxamide (23 mg, 0.048 mmol) in DCM (2 mL) was added acetyl acetate (7 μL, 0.074 mmol), DMAP (2 mg, 0.016 mmol) and NEt₃ (14 μL, 0.10 mmol). The reaction was stirred for 1 hour before being quenched with MeOH and partioned between saturated aqueous NH₄Cl solution and DCM. The organic layer was passed through a phase separator cartridge, washing with DCM, and the filtrate concentrated in vacuo to give rac-1-(4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)ethyl acetate (23 mg, 92%). ESI-MS m/z calc. 516.16833, found 517.6 (M+1)⁺; 515.7 (M−1)⁻.

### Step 3:

Purification of rac-1-(4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)ethyl acetate (23 mg, 0.04453 mmol) by chiral SFC [System: (R,R)-Whelk-01 column, 5 μm particle size, 25 cm×21.2 mm from Regis Technologies] gave:

First eluting isomer (rt=2.47 mins): rel-1-(4-((2S,3R,4R,5S)-3-(3,4-difluoro-2-methoxyphenyl)-4,5-dimethyl-5-(trifluoromethyl)tetrahydrofuran-2-carboxamido)pyridin-2-yl)ethyl acetate (7 mg). ESI-MS m/z calc. 516.16833, found 517.5 (M+1)⁺; 515.5 (M−1)⁻.

Second eluting isomer (rt=4.34 mins): rel-1-(4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)ethyl acetate (7 mg). ESI-MS m/z calc. 516.16833, found 517.5 (M+1)⁺; 515.5 (M−1)⁻.

### Step 4:

First Eluting Peak from Previous Step:

To a solution of rel-1-(4-((2S,3R,4R,5S)-3-(3,4-difluoro-2-methoxyphenyl)-4,5-dimethyl-5-(trifluoromethyl)tetrahydrofuran-2-carboxamido)pyridin-2-yl)ethyl acetate (7 mg, 0.014 mmol) in MeOH (3 mL) was added K₂CO₃ (9 mg, 0.065 mmol). The reaction was stirred at ambient temperature for 1.5 hours, then diluted with water and extracted with EtOAc (×3) The combined organic layers were dried (MgSO₄) and concentrated in vacuo to give rel-(2S,3R,4R,5S)-3-(3,4-difluoro-2-methoxyphenyl)-N-(2-(1-hydroxyethyl)pyridin-4-yl)-4,5-dimethyl-5-(trifluoromethyl)tetrahydrofuran-2-carboxamide (531, 4.8 mg, 66%). ESI-MS m/z calc. 474.1578, found 476.5 (M+1)⁺; 473.7 (M−1)⁻.

Second Eluting Peak from Previous Step:

To a solution of rel-1-(4-((2R,3S,4S,5R)-3-(3,4-difluoro-2-methoxyphenyl)-4,5-dimethyl-5-(trifluoromethyl)tetrahydrofuran-2-carboxamido)pyridin-2-yl)ethyl acetate (7 mg, 0.014 mmol) in MeOH (3 mL) was added K₂CO₃ (9 mg, 0.065 mmol). The reaction was stirred at ambient temperature for 1.5 hours, then diluted with water and extracted with EtOAc (×3) The combined organic layers were dried (MgSO₄) and concentrated in vacuo to give rel-(2R,3S,4S,5R)-3-(3,4-difluoro-2-methoxyphenyl)-N-(2-(1-hydroxyethyl)pyridin-4-yl)-4,5-dimethyl-5-(trifluoromethyl)tetrahydrofuran-2-carboxamide (532, 5.0 mg, 75%). ¹H NMR (500 MHz, Chloroform-d) δ 8.46 (s, 1H), 8.43 (d, J=5.6 Hz, 1H), 7.54 (d, J=2.0 Hz, 1H), 7.35 (dd, J=5.6, 2.1 Hz, 1H), 7.08 (ddd, J=8.1, 5.5, 2.1 Hz, 1H), 6.91 (td, J=9.2, 7.4 Hz, 1H), 5.01 (d, J=11.0 Hz, 1H), 4.83 (q, J=6.5 Hz, 1H), 4.09 (dd, J=11.0, 8.1 Hz, 1H), 4.00 (d, J=2.7 Hz, 3H), 2.75 (p, J=7.7 Hz, 1H), 1.68 (d, J=1.2 Hz, 3H), 1.48 (d, J=6.5 Hz, 3H), 0.79 (dq, J=7.4, 2.4 Hz, 3H) ppm; ESI-MS m/z calc. 474.1578, found 475.8 (M+1)⁺; 473.7 (M−1)⁻.

The following compounds were made using a method similar to that described in Example 14 except the other enantiomer of 1-(4-bromo-2-pyridyl)ethanol (second eluting isomer by SFC using a Chiralpak ID column) was used in step 1. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 533 | rel-(2S,3R,4R,5S)-3- | 474.421 | 475.3 | 3.23 | ¹H NMR (500 MHz, |
| | (3,4-difluoro-2- | | | | Chloroform-d) δ 8.45 (s, 1H), |
| | methoxyphenyl)-N-(2- | | | | 8.43 (d, J = 5.6 Hz, 1H), 7.54 |
| | (1-hydroxyethyl)pyridin- | | | | (d, J = 2.0 Hz, 1H), 7.35 (dd, |
| | 4-yl)-4,5-dimethyl-5- | | | | J = 5.6, 2.1 Hz, 1H), 7.08 |
| | (trifluoromethyl)tetrahydrofuran- | | | | (ddd, J = 8.2, 5.5, 2.0 Hz, |
| | 2-carboxamide | | | | 1H), 6.91 (td, J = 9.2, 7.4 Hz, |
| | (precursor was first | | | | 1H), 5.01 (d, J = 11.0 Hz, |
| | eluting isomer by SFC | | | | 1H), 4.83 (q, J = 6.5 Hz, 1H), |
| | using Whelk-O1 | | | | 4.09 (dd, J = 11.0, 8.0 Hz, |
| | column) | | | | 1H), 4.00 (d, J = 2.8 Hz, 3H), |
| | | | | | 2.75 (p, J = 7.7 Hz, 1H), 1.78- |
| | | | | | 1.66 (m, 3H), 1.48 (d, J = |
| | | | | | 6.6 Hz, 3H), 0.79 (dq, J = |
| | | | | | 7.5, 2.3 Hz, 3H) ppm. |
| 534 | rel-(2R,3S,4S,5R)-3- | 474.421 | 475.6 | 3.23 | ¹H NMR (500 MHz, |
| | (3,4-difluoro-2- | | | | Chloroform-d) δ 10.39 (s, |
| | methoxyphenyl)-N-(2- | | | | 1H), 8.48 (d, J = 6.3 Hz, 1H), |
| | (1-hydroxyethyl)pyridin- | | | | 8.13 (d, J = 6.4 Hz, 1H), 8.10 |
| | 4-yl)-4,5-dimethyl-5- | | | | (s, 1H), 7.19-7.12 (m, 1H), |
| | (trifluoromethyl)tetrahydrofuran- | | | | 6.97-6.85 (m, 1H), 5.24 (d, |
| | 2-carboxamide | | | | J = 10.9 Hz, 1H), 5.14 (d, J = |
| | (precursor was second | | | | 6.7 Hz, 1H), 4.24 (dd, J = |
| | eluting isomer by SFC | | | | 10.9, 8.0 Hz, 1H), 4.01 (d, J = |
| | using Whelk-O1 | | | | 2.6 Hz, 3H), 2.77 (p, J = |
| | column) | | | | 7.6 Hz, 1H), 1.70 (s, 3H), |
| | | | | | 1.55 (d, J = 6.4 Hz, 3H), 0.83- |
| | | | | | 0.75 (m, 3H). ppm |

### Example 15

(2R,3S,4S,5R)-3-(3,4-difluoro-2-methoxyphenyl)-N-(5-((R)-2,4-dimethyl-6-oxopiperazin-1-yl)pyridin-3-yl)-4,5-dimethyl-5-(trifluoromethyl)tetrahydrofuran-2-carboxamide (535)

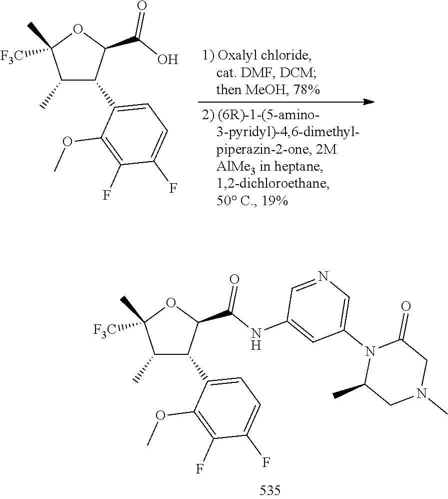 [SMILES: COc1c([C@H]2[C@H](C(=O)O)O[C@@](C)(C(F)(F)F)[C@H]2C)ccc(F)c1F ; COc1c([C@H]2[C@H](C(=O)Nc3cncc(N4C(=O)CN(C)C[C@H]4C)c3)O[C@@](C)(C(F)(F)F)[C@H]2C)ccc(F)c1F]

### Step 1

A solution of (2R,3S,4S,5R)-3-(3,4-difluoro-2-methoxyphenyl)-4,5-dimethyl-5-(trifluoromethyl)tetrahydrofuran-2-carboxylic acid (204 mg, 0.5758 mmol) and DMF (3 mg, 0.0032 mL, 0.0410 mmol) in dichloromethane (5 mL) was treated with oxalyl chloride (97 mg, 0.0667 mL, 0.7642 mmol) in dichloromethane (1 mL) and was stirred for 3 hours at ambient temperature. The solvent was removed under reduced pressure, methanol (791.00 mg, 1 mL, 24.686 mmol) was added, and after stirring for 5 minutes at room temperature, the solvent was evaporated under reduced pressure to give methyl (2R,3S,4S,5R)-3-(3,4-difluoro-2-methoxy-phenyl)-4,5-dimethyl-5-(trifluoromethyl)tetrahydrofuran-2-carboxylate (177 mg, 78%) as a colourless oil. ¹H NMR (400 MHz, Chloroform-d) δ 6.93-6.81 (m, 2H), 4.90 (d, J=10.1 Hz, 1H), 4.12 (dd, J=10.1, 7.8 Hz, 1H), 3.99 (d, J=2.7 Hz, 3H), 3.70 (s, 3H), 2.75-2.67 (m, 1H), 1.61 (s, 3H), 0.77-0.73 (m, 3H) ppm.

### Step 2

To a stirred solution of (6R)-1-(5-amino-3-pyridyl)-4,6-dimethyl-piperazin-2-one (13.3 mg, 0.0574 mmol) and methyl (2R,3S,4S,5R)-3-(3,4-difluoro-2-methoxy-phenyl)-4,5-dimethyl-5-(trifluoromethyl)tetrahydrofuran-2-carboxylate (24 mg, 0.0652 mmol) in 1,2-dichloroethane (2 mL) was added 2 M trimethylaluminium in heptane (58 μL of 2 M, 0.1160 mmol) and the reaction mixture was stirred at room temperature for 1 hour. Another portion of 2 M trimethylaluminium in heptane (50 μL of 2 M, 0.1000 mmol) was added and the reaction mixture was heated at 50° C. for 30 min. A third portion of 2 M trimethylaluminium in heptane (50 μL of 2 M, 0.1000 mmol) was added and the reaction mixture was heated at 60° C. for 15 min. The reaction mixture was stood over the weekend at ambient temperature. The reaction mixture was heated to 50° C. and another portion of 2 M trimethylaluminium in heptane (100 μL of 2 M, 0.2000 mmol) was added. After 30 min, both methyl (2R,3S,4S,5R)-3-(3,4-difluoro-2-methoxy-phenyl)-4,5-dimethyl-5-(trifluoromethyl)tetrahydrofuran-2-carboxylate (20 mg, 0.0543 mmol) and 2 M trimethylaluminium in heptane (50 μL of 2 M, 0.1000 mmol) were added and heating was continued for 30 min at 50° C. The reaction mixture was cooled to room temperature and was treated with 2N aqueous hydrochloric acid (2 mL) then with 2N aqueous sodium hydroxide solution (3 mL). The reaction mixture was extracted with ethyl acetate (15 mL), dried over sodium sulfate and the crude material was purified by flash column chromatography (4 g SiO₂, 0 to 10% methanol in DCM). The partially purified material was purified by catch and release (0.5 g, 3 mL SCX-2 cartridge) using methanol then 15% ammonia in methanol and then lyopholised to give (2R,3S,4S,5R)-3-(3,4-difluoro-2-methoxyphenyl)-N-(5-((R)-2,4-dimethyl-6-oxopiperazin-1-yl)pyridin-3-yl)-4,5-dimethyl-5-(trifluoromethyl)tetrahydrofuran-2-carboxamide (535, 6.62 mg, 19%) as a white solid. ¹H NMR (400 MHz, Chloroform-d) δ 8.57 (s, 1H), 8.53 (s, 1H), 8.24 (d, J=1.8 Hz, 1H), 8.10 (t, J=2.3 Hz, 1H), 7.09-7.04 (m, 1H), 6.88 (td, J=9.3, 7.6 Hz, 1H), 5.00 (d, J=11.0 Hz, 1H), 4.08 (dd, J=11.0, 7.8 Hz, 2H), 4.00 (d, J=3.2 Hz, 3H), 3.30 (s, 2H), 2.98 (d, J=9.2 Hz, 1H), 2.74 (q, J=7.6 Hz, 1H), 2.57 (q, J=6.1 Hz, 1H), 2.44 (s, 3H), 1.67 (s, 3H), 1.10 (d, J=6.4 Hz, 3H), 0.78-0.76 (m, 3H) ppm. ESI-MS m/z calc. 556.2109, found 557.24 (M+1)⁺; Retention time: 2.44 minutes.

### Example 16

(2R,3S,4S,5R)-3-(3,4-difluoro-2-(2-hydroxyethoxy)phenyl)-N-(1-(difluoromethyl)-3-methyl-1H-pyrazol-4-yl)-4,5-dimethyl-5-(trifluoromethyl)tetrahydrofuran-2-carboxamide (536)

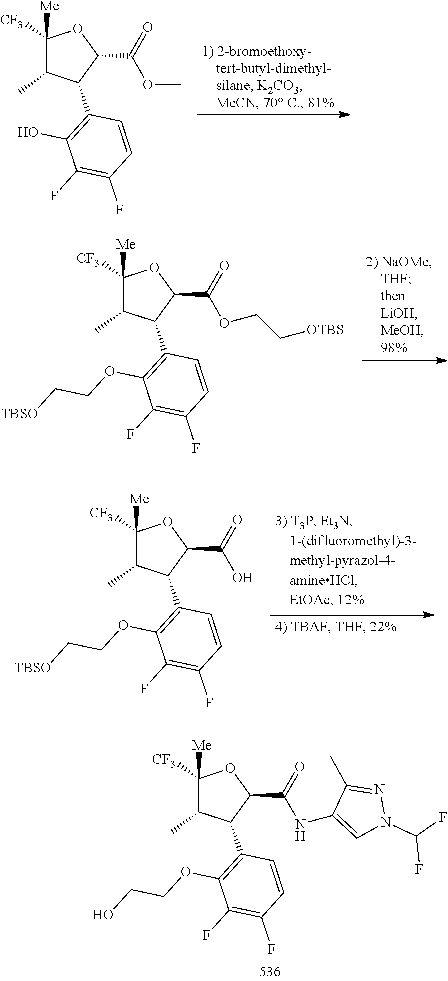

### Step 1:

To a mixture of methyl (2S,3S,4S,5R)-3-(3,4-difluoro-2-hydroxy-phenyl)-4,5-dimethyl-5-(trifluoromethyl)tetrahydrofuran-2-carboxylate (3 g, 8.468 mmol) and K₂CO₃ (3.7 g, 26.77 mmol) in MeCN (30 mL) was added 2-bromoethoxy-tert-butyl-dimethyl-silane (5.5 mL, 25.63 mmol) and the resulting mixture heated at about 73° C. under nitrogen overnight. K₂CO₃ (3.7 g, 26.77 mmol) and 2-bromoethoxy-tert-butyl-dimethyl-silane (5.5 mL, 25.63 mmol) were added and the reaction heated at 70° C. K₂CO₃ (3.7 g, 26.77 mmol) and 2-bromoethoxy-tert-butyl-dimethyl-silane (5.5 mL, 25.63 mmol) were added and the reaction mixture heated at 70° C. over the weekend. The mixture was cooled to ambient temperature then filtered through a celite cartridge. The filtrate was concentrated in vacuo and then dissolved in MTBE. The organic layer was washed with water (×2) and brine. The organic phase was dried (Na₂SO₄), filtered and concentrated in vacuo. Purification by flash chromatography (SiO₂, 0 to 20% EtOAc in heptane) gave 2-[tert-butyl(dimethyl)silyl]oxyethyl (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylate (3.9 g, 70%) as a clear oil. ¹H NMR (500 MHz, DMSO-d₆) δ 7.18 (ddd, J=8.0, 5.8, 1.8 Hz, 1H), 7.10 (dt, J=9.8, 8.3 Hz, 1H), 5.12 (d, J=10.4 Hz, 1H), 4.25-4.13 (m, 3H), 4.10-4.04 (m, 2H), 3.93-3.86 (m, 2H), 3.70-3.62 (m, 2H), 2.73 (q, J=7.5 Hz, 1H), 1.53 (s, 3H), 0.86 (s, 9H), 0.80 (s, 9H), 0.69 (dt, J=8.5, 4.3 Hz, 3H), 0.06 (s, 6H), −0.02 (d, J=2.2 Hz, 6H) ppm.

### Step 2:

Sodium methoxide (120.5 μL of 25% w/v, 0.5576 mmol) was added to a solution of 2-[tert-butyl(dimethyl)silyl]oxyethyl (2S,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylate (3.9 g, 5.937 mmol) in THF (30 mL). The reaction mixture was stirred at ambient temperature for 5 h. MeOH (30 mL) and LiOH (3.614 mL of 2 M aqueous solution, 7.228 mmol) were added and the reaction mixture was stirred overnight. The reaction mixture was poured onto 1M HCl and then extracted with MTBE (2×30 mL). The combined organic extracts were washed with brine, dried (Na₂SO₄), filtered and concentrated in vacuo to give (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylic acid (2.89 g, 98%). ESI-MS m/z calc. 498.1861, found 499.6 (M+1)⁺; 497.6 (M−1)⁻; Retention time: 0.82 minutes.

### Step 3

To a solution of (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylic acid (27 mg, 0.054 mmol) in EtOAc (0.75 mL) was added T3P solution (80 μL of 50% w/v in EtOAc, 0.1257 mmol) and Et₃N (40 μL, 0.2870 mmol). The reaction was stirred at ambient temperature for 15 min. Then 1-(difluoromethyl)-3-methyl-pyrazol-4-amine (HCl salt) (17 mg, 0.09260 mmol) was added and the reaction mixture was stirred at ambient temperature over the weekend. The reaction mixture was partitioned between EtOAc (6 mL) and water (3 mL). The organic layer was dried (phase separation cartridge) and concentrated in vacuo. Purification by flash chromatography (SiO₂, 0 to 100% EtOAc in heptane) gave (2R,3S,4S,5R)-3-[2-[2-[tert-butyl(dimethyl)silyl]oxyethoxy]-3,4-difluoro-phenyl]-N-[1-(difluoromethyl)-3-methyl-pyrazol-4-yl]-4,5-dimethyl-5-(trifluoromethyl)tetrahydrofuran-2-carboxamide (4 mg, 12%) as a pale yellow oil. ESI-MS m/z calc. 627.2363, found 628.8 (M+1)⁺; Retention time: 1.26 minutes.

### Step 4

To a solution of (2R,3S,4S,5R)-3-[2-[2-[tert-butyl(dimethyl)silyl]oxyethoxy]-3,4-difluoro-phenyl]-N-[1-(difluoromethyl)-3-methyl-pyrazol-4-yl]-4,5-dimethyl-5-(trifluoromethyl)tetrahydrofuran-2-carboxamide (4 mg, 0.006373 mmol) in 2-MeTHF (500 μL) at 0° C. was added TBAF (20 μL of 1 M solution in THF, 0.020 mmol). The reaction mixture was stirred at 0° C. and then at ambient temperature for 4 h. The reaction mixture was quenched with saturated aqueous NaHCO₃ (1 mL), stirred for 5 minutes and extracted with EtOAc (2×3 mL). The combined organic extracts were dried (phase separation cartridge) and concentrated in vacuo. Purification by preparative reverse phase HPLC (basic eluent) gave (2R,3S,4S,5R)-3-(3,4-difluoro-2-(2-hydroxyethoxy)phenyl)-N-(1-(difluoromethyl)-3-methyl-1H-pyrazol-4-yl)-4,5-dimethyl-5-(trifluoromethyl)tetrahydrofuran-2-carboxamide (536, 1.5 mg, 22% o) as a beige solid. ¹H NMR (500 μMHz, DMSO-d₆) δ 9.92 (s, 1H), 8.30 (s, 1H), 7.63 (t, J=59.2 Hz, 1H), 7.20-7.13 (m, 2H), 5.17 (d, 1=10.8 Hz, 1H), 4.97 (t, 1=5.2 Hz, 1H), 4.36 (dd, =10.7, 7.1 Hz, 1H), 4.11 (ddt, J=19.2, 9.7, 4.9 Hz, 2H), 3.72 (q, ==4.9 Hz, 2H), 2.93 (p, 5=7.4 Hz, 1H), 2.17 (s, 3H), 1.59 (s, 3H), 0.75-0.69 (in, 3H) ppm. ⁹F NMR (471 MHz, DMSO-d₆) δ−73.60, −93.96 (d, =59.3 Hz), −138.41 (d, J=20.2 Hz), −154.47 (d, J=22.5 Hz) ppm. ESI-MS m/z calc. 513.14984, found 514.6 (M+1); Retention time: 3.17 minutes.

The following compounds were made using a method similar to that described in Example 16, except that different amines were used in amide coupling step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 537 | (2R,3S,4S,5R)-3-(3,4- | 490.42 | 491.7 | 2.8 | ¹H NMR (500 MHz, DMSO- |
| | difluoro-2-(2- | | | | d₆) δ 10.56 (s, 1H), 8.36 (d, J = |
| | hydroxyethoxy)phenyl)- | | | | 5.5 Hz, 1H), 7.75 (d, J = |
| | N-(2-(hydroxymethyl)pyridin- | | | | 2.1 Hz, 1H), 7.53 (dd, J = |
| | 4-yl)-4,5-dimethyl-5- | | | | 5.6, 2.2 Hz, 1H), 7.19 (dd, J = |
| | (trifluoromethyl)tetrahydrofuran- | | | | 9.5, 6.3 Hz, 2H), 5.41 (s, |
| | 2-carboxamide | | | | 1H), 5.13 (d, J = 10.7 Hz, |
| | | | | | 1H), 4.60-4.49 (m, 2H), |
| | | | | | 4.43 (dd, J = 10.7, 7.3 Hz, |
| | | | | | 1H), 4.20-4.09 (m, 2H), |
| | | | | | 3.75 (t, J = 4.6 Hz, 2H), 3.20 |
| | | | | | (s, 1H), 2.97 (p, J = 7.4 Hz, |
| | | | | | 1H), 1.64 (s, 3H), 0.75 (d, J = |
| | | | | | 7.3 Hz, 3H) ppm. |
| 538 | (2R,3S,4S,5R)-3-(3,4- | 541.489 | 542.6 | 3.07 | ¹H NMR (500 MHz, DMSO- |
| | difluoro-2-(2- | | | | d₆) δ 10.06 (s, 1H), 8.34 (s, |
| | hydroxyethoxy)phenyl)- | | | | 1H), 7.20-7.12 (m, 2H), |
| | 4,5-dimethyl-N-(3- | | | | 5.20 (d, J = 10.8 Hz, 1H), |
| | methyl-1- | | | | 4.97 (s, 1H), 4.38 (dd, J = |
| | (methylsulfonyl)-1H- | | | | 10.9, 7.2 Hz, 1H), 4.11 (dp, J = |
| | pyrazol-4-yl)-5- | | | | 19.2, 4.9 Hz, 2H), 3.72 (q, |
| | (trifluoromethyl)tetrahydrofuran- | | | | J = 4.8 Hz, 2H), 3.43 (s, 3H), |
| | 2-carboxamide | | | | 2.94 (p, J = 7.4 Hz, 1H), 2.25 |
| | | | | | (s, 3H), 1.59 (s, 3H), 0.75- |
| | | | | | 0.69 (m, 3H) ppm. |
| 539 | (2R,3S,4S,5R)-3-(3,4- | 461.383 | 462.7 | 2.83 | ¹H NMR (500 MHz, DMSO- |
| | difluoro-2-(2- | | | | d₆) δ 10.45 (s, 1H), 9.01 (s, |
| | hydroxyethoxy)phenyl)- | | | | 2H), 8.91 (s, 1H), 7.25-7.08 |
| | 4,5-dimethyl-N- | | | | (m, 2H), 5.15 (d, J = 10.8 Hz, |
| | (pyrimidin-5-yl)-5- | | | | 1H), 4.98 (s, 1H), 4.42 (dd, J = |
| | (trifluoromethyl)tetrahydrofuran- | | | | 10.8, 7.3 Hz, 1H), 4.19- |
| | 2-carboxamide | | | | 4.04 (m, 2H), 3.69 (s, 2H), |
| | | | | | 2.95-2.84 (m, 1H), 1.62 (s, |
| | | | | | 3H), 0.71 (d, J = 7.4 Hz, 3H) |
| | | | | | ppm. |
| 540 | (2R,3S,4S,5R)-3-(3,4- | 518.474 | 519.8 | 3.00 | ¹H NMR (500 MHz, DMSO- |
| | difluoro-2-(2- | | | | d₆) δ 10.55 (s, 1H), 8.34 (d, J = |
| | hydroxyethoxy)phenyl)- | | | | 5.5 Hz, 1H), 7.88 (d, J = |
| | N-(2-(2-hydroxypropan- | | | | 2.0 Hz, 1H), 7.48 (dd, J = |
| | 2-yl)pyridin-4-yl)-4,5- | | | | 5.5, 2.1 Hz, 1H), 7.21-7.09 |
| | dimethyl-5- | | | | (m, 2H), 5.20 (s, 1H), 5.16- |
| | (trifluoromethyl)tetrahydrofuran- | | | | 4.92 (m, 2H), 4.39 (dd, J = |
| | 2-carboxamide | | | | 10.8, 7.3 Hz, 1H), 4.18-4.05 |
| | | | | | (m, 2H), 3.72 (t, J = 4.6 Hz, |
| | | | | | 2H), 2.93 (p, J = 7.5 Hz, 1H), |
| | | | | | 1.60 (s, 3H), 1.40 (s, 6H), |
| | | | | | 0.76-0.62 (m, 3H) ppm. |
| 541 | (2R,3S,4S,5R)-3-(3,4- | 490.42 | 491.2 | 2.75 | |
| | difluoro-2-(2- | | | | |
| | hydroxyethoxy)phenyl)- | | | | |
| | 4,5-dimethyl-N-(1- | | | | |
| | methyl-2-oxo-1,2- | | | | |
| | dihydropyridin-4-yl)-5- | | | | |
| | (trifluoromethyl)tetrahydrofuran- | | | | |
| | 2-carboxamide | | | | |
| 542 | (2R,3S,4S,5R)-3-(3,4- | 546.487 | 547.3 | 3.05 | ¹H NMR (500 MHz, DMSO- |
| | difluoro-2-(2- | | | | d₆) δ 10.00 (s, 1H), 8.53 (s, |
| | hydroxyethoxy)phenyl)- | | | | 2H), 7.24-7.08 (m, 2H), |
| | 4,5-dimethyl-N-(2- | | | | 5.06 (d, J = 10.8 Hz, 1H), |
| | morpholinopyrimidin-5- | | | | 4.96 (s, 1H), 4.36 (dd, J = |
| | yl)-5- | | | | 10.8, 7.3 Hz, 1H), 4.13 (dt, J = |
| | (trifluoromethyl)tetrahydrofuran- | | | | 9.6, 4.6 Hz, 1H), 4.08 (dt, J = |
| | 2-carboxamide | | | | 9.8, 4.3 Hz, 1H), 3.70 (t, J = |
| | | | | | 4.7 Hz, 2H), 3.63 (s, 8H), |
| | | | | | 2.88 (p, J = 7.4 Hz, 1H), 1.60 |
| | | | | | (s, 3H), 0.76-0.65 (m, 3H) |
| | | | | | ppm. |
| 543 | (2R,3S,4S,5R)-3-(3,4- | 489.436 | 490.3 | 2.84 | ¹H NMR (500 MHz, DMSO- |
| | difluoro-2-(2- | | | | d₆) δ 9.78 (s, 1H), 8.51 (s, |
| | hydroxyethoxy)phenyl)- | | | | 1H), 7.26 (t, J = 7.4 Hz, 1H), |
| | N-(2,4- | | | | 7.16 (q, J = 8.9 Hz, 1H), 5.17 |
| | dimethylpyrimidin-5- | | | | (d, J = 10.9 Hz, 1H), 4.97 (s, |
| | yl)-4,5-dimethyl-5- | | | | 1H), 4.38 (dd, J = 10.9, 7.2 |
| | (trifluoromethyl)tetrahydrofuran- | | | | Hz, 1H), 4.18-4.10 (m, 1H), |
| | 2-carboxamide | | | | 4.09-4.02 (m, 1H), 3.74- |
| | | | | | 3.63 (m, 2H), 2.87 (p, J = 7.4 |
| | | | | | Hz, 1H), 2.53 (s, 3H), 2.22 |
| | | | | | (s, 3H), 1.63 (s, 3H), 0.76- |
| | | | | | 0.67 (m, 3H) ppm. |
| 544 | (2R,3S,4S,5R)-3-(3,4- | 490.42 | 491.2 | 3.17 | ¹H NMR (500 MHz, DMSO- |
| | difluoro-2-(2- | | | | d₆) δ 10.45 (s, 1H), 8.04 (d, J = |
| | hydroxyethoxy)phenyl)- | | | | 5.7 Hz, 1H), 7.21-7.08 |
| | N-(2-methoxypyridin-4- | | | | (m, 4H), 5.07 (d, J = 10.7 Hz, |
| | yl)-4,5-dimethyl-5- | | | | 1H), 4.38 (dd, J = 10.7, 7.3 |
| | (trifluoromethyl)tetrahydrofuran- | | | | Hz, 1H), 4.17-4.06 (m, 2H), |
| | 2-carboxamide | | | | 3.81 (s, 3H), 3.71 (t, J = 4.6 |
| | | | | | Hz, 2H), 2.92 (p, J = 7.4 Hz, |
| | | | | | 1H), 1.59 (s, 3H), 0.74-0.66 |
| | | | | | (m, 3H) ppm. |
| 545 | (2R,3S,4S,5R)-3-(3,4- | 464.383 | 465.59 | 3.03 | |
| | difluoro-2-(2- | | | | |
| | hydroxyethoxy)phenyl)- | | | | |
| | 4,5-dimethyl-N-(4- | | | | |
| | methylisoxazol-3-yl)-5- | | | | |
| | (trifluoromethyl)tetrahydrofuran- | | | | |
| | 2-carboxamide | | | | |
| 546 | (2R,3S,4S,5R)-N-(5- | 503.462 | 504.6 | 3.19 | |
| | cyclopropyl-1-methyl- | | | | |
| | 1H-pyrazol-3-yl)-3-(3,4- | | | | |
| | difluoro-2-(2- | | | | |
| | hydroxyethoxy)phenyl)- | | | | |
| | 4,5-dimethyl-5- | | | | |
| | (trifluoromethyl)tetrahydrofuran- | | | | |
| | 2-carboxamide | | | | |
| 547 | (2R,3S,4S,5R)-3-(3,4- | 463.398 | 464.24 | 2.86 | |
| | difluoro-2-(2- | | | | |
| | hydroxyethoxy)phenyl)- | | | | |
| | 4,5-dimethyl-N-(1- | | | | |
| | methyl-1H-pyrazol-5- | | | | |
| | yl)-5- | | | | |
| | (trifluoromethyl)tetrahydrofuran- | | | | |
| | 2-carboxamide | | | | |
| 548 | (2R,3S,4S,5R)-N- | 500.419 | 501.2 | 2.75 | ¹H NMR (500 MHz, DMSO- |
| | ([1,2,4]triazolo[4,3- | | | | d₆) δ 10.43 (s, 1H), 9.28 (d, J = |
| | alpyridin-6-yl)-3-(3,4- | | | | 14.6 Hz, 2H), 7.81 (d, J = |
| | difluoro-2-(2- | | | | 9.9 Hz, 1H), 7.47 (s, 1H), |
| | hydroxyethoxy)phenyl)- | | | | 7.16 (dd, J = 8.6, 4.9 Hz, |
| | 4,5-dimethyl-5- | | | | 2H), 5.13 (d, J = 10.6 Hz, |
| | (trifluoromethyl)tetrahydrofuran- | | | | 1H), 4.42 (dd, J = 10.7, 7.3 |
| | 2-carboxamide | | | | Hz, 1H), 4.11 (dp, J = 19.5, |
| | | | | | 4.9 Hz, 2H), 3.71 (t, J = 4.8 |
| | | | | | Hz, 2H), 2.92 (p, J = 7.7 Hz, |
| | | | | | 1H), 1.62 (s, 3H), 0.73 (d, J = |
| | | | | | 7.3 Hz, 3H) ppm. |
| 549 | (2R,3S,4S,5R)-N-(1- | 488.408 | 489.2 | 2.92 | |
| | (cyanomethyl)-1H- | | | | |
| | pyrazol-4-yl)-3-(3,4- | | | | |
| | difluoro-2-(2- | | | | |
| | hydroxyethoxy)phenyl)- | | | | |
| | 4,5-dimethyl-5- | | | | |
| | (trifluoromethyl)tetrahydrofuran- | | | | |
| | 2-carboxamide | | | | |
| 550 | (2R,3S,4S,5R)-3-(3,4- | 491.408 | 492.2 | 2.95 | |
| | difluoro-2-(2- | | | | |
| | hydroxyethoxy)phenyl)- | | | | |
| | N-(2-methoxypyrimidin- | | | | |
| | 5-yl)-4,5-dimethyl-5- | | | | |
| | (trifluoromethyl)tetrahydrofuran- | | | | |
| | 2-carboxamide | | | | |
| 551 | (2R,3S,4S,5R)-3-(3,4- | 544.511 | 545.22 | 2.99 | |
| | difluoro-2-(2- | | | | |
| | hydroxyethoxy)phenyl)- | | | | |
| | 4,5-dimethyl-N-(2- | | | | |
| | methyl-5- | | | | |
| | (tetrahydrofuran-3- | | | | |
| | yl)pyridin-3-yl)-5- | | | | |
| | (trifluoromethyl)tetrahydrofuran- | | | | |
| | 2-carboxamide | | | | |
| | as a mixture of epimers | | | | |
| | on the stereocenter of | | | | |
| | the tetrahydrofuran of | | | | |
| | the tetrahydrofuran-3-yl- | | | | |
| | pyridinyl | | | | |
| 552 | (2R,3S,4S,5R)-3-(3,4- | 507.451 | 508.65 | 2.97 | |
| | difluoro-2-(2- | | | | |
| | hydroxyethoxy)phenyl)- | | | | |
| | N-(1-(2-methoxyethyl)- | | | | |
| | 1H-pyrazol-3-yl)-4,5- | | | | |
| | dimethyl-5- | | | | |
| | (trifluoromethyl)tetrahydrofuran- | | | | |
| | 2-carboxamide | | | | |
| 553 | (2R,3S,4S,5R)-3-(3,4- | 494.412 | 495.25 | 2.71 | ¹H NMR (500 MHz, DMSO- |
| | difluoro-2-(2- | | | | d₆) δ 10.68 (s, 1H), 8.58 (s, |
| | hydroxyethoxy)phenyl)- | | | | 1H), 7.17 (dd, J = 8.6, 5.1 |
| | N-(1-(methoxymethyl)- | | | | Hz, 2H), 5.36 (s, 2H), 5.09 |
| | 1H-1,2,4-triazol-3-yl)- | | | | (d, J = 10.8 Hz, 1H), 4.34 |
| | 4,5-dimethyl-5- | | | | (dd, J = 10.9, 7.2 Hz, 1H), |
| | (trifluoromethyl)tetrahydrofuran- | | | | 4.16-4.06 (m, 2H), 3.72 (dd, |
| | 2-carboxamide | | | | J = 5.3, 4.0 Hz, 2H), 3.25 (s, |
| | | | | | 3H), 2.91 (p, J = 7.4 Hz, 1H), |
| | | | | | 1.58 (s, 3H), 0.74-0.62 (m, |
| | | | | | 3H) ppm. |
| 554 | (2R,3S,4S,5R)-3-(3,4- | 522.441 | 523.56 | 2.91 | |
| | difluoro-2-(2- | | | | |
| | hydroxyethoxy)phenyl)- | | | | |
| | N-(1-((15,3S)-3- | | | | |
| | fluorocyclobutyl)-1H- | | | | |
| | 1,2,4-triazol-3-yl)-4,5- | | | | |
| | dimethyl-5- | | | | |
| | (trifluoromethyl)tetrahydrofuran- | | | | |
| | 2-carboxamide | | | | |
| 555 | (2R,3S,4S,5R)-3-(3,4- | 466.422 | 467.14 | 2.94 | |
| | difluoro-2-(2- | | | | |
| | hydroxyethoxy)phenyl)- | | | | |
| | 4,5-dimethyl-N-(thiazol- | | | | |
| | 5-yl)-5- | | | | |
| | (trifluoromethyl)tetrahydrofuran- | | | | |
| | 2-carboxamide | | | | |
| 556 | (2R,3S,4S,5R)-3-(3,4- | 450.357 | 451.48 | 3.01 | |
| | difluoro-2-(2- | | | | |
| | hydroxyethoxy)phenyl)- | | | | |
| | N-(isoxazol-3-yl)-4,5- | | | | |
| | dimethyl-5- | | | | |
| | (trifluoromethyl)tetrahydrofuran- | | | | |
| | 2-carboxamide | | | | |
| 557 | (2R,3S,4S,5R)-3-(3,4- | 477.425 | 478.24 | 2.86 | |
| | difluoro-2-(2- | | | | |
| | hydroxyethoxy)phenyl)- | | | | |
| | N-(1,3-dimethyl-1H- | | | | |
| | pyrazol-4-yl)-4,5- | | | | |
| | dimethyl-5- | | | | |
| | (trifluoromethyl)tetrahydrofuran- | | | | |
| | 2-carboxamide | | | | |
| 558 | (2R,3S,4S,5R)-3-(3,4- | 464.383 | 465.24 | 3.04 | |
| | difluoro-2-(2- | | | | |
| | hydroxyethoxy)phenyl)- | | | | |
| | 4,5-dimethyl-N-(3- | | | | |
| | methylisoxazol-4-yl)-5- | | | | |
| | (trifluoromethyl)tetrahydrofuran- | | | | |
| | 2-carboxamide | | | | |
| 559 | (2R,3S,4S,5R)-3-(3,4- | 464.383 | 465.19 | 3.02 | |
| | difluoro-2-(2- | | | | |
| | hydroxyethoxy)phenyl)- | | | | |
| | 4,5-dimethyl-N-(5- | | | | |
| | methylisoxazol-4-yl)-5- | | | | |
| | (trifluoromethyl)tetrahydrofuran- | | | | |
| | 2-carboxamide | | | | |
| 560 | (2R,3S,4S,5R)-3-(3,4- | 474.421 | 475.29 | 3.01 | ¹H NMR (500 MHz, DMSO- |
| | difluoro-2-(2- | | | | d₆) δ 11.05 (s, 1H), 8.55 (d, J = |
| | hydroxyethoxy)phenyl)- | | | | 6.9 Hz, 1H), 7.96 (s, 1H), |
| | 4,5-dimethyl-N-(2- | | | | 7.85 (s, 1H), 7.22-7.10 (m, |
| | methylpyridin-4-yl)-5- | | | | 2H), 5.19 (d, J = 10.5 Hz, |
| | (trifluoromethyl)tetrahydrofuran- | | | | 1H), 4.43 (dd, J = 10.5, 7.4 |
| | 2-carboxamide | | | | Hz, 1H), 4.18-4.04 (m, 2H), |
| | | | | | 3.72-3.66 (m, 2H), 2.92 (p, |
| | | | | | J = 7.5 Hz, 1H), 2.58 (s, 3H), |
| | | | | | 1.61 (s, 3H), 0.76-0.70 (m, |
| | | | | | 3H) ppm. |
| 561 | (2R,3S,4S,5R)-3-(3,4- | 520.45 | 521.26 | 2.7 | |
| | difluoro-2-(2- | | | | |
| | hydroxyethoxy)phenyl)- | | | | |
| | 4,5-dimethyl-N-(1- | | | | |
| | (tetrahydrofuran-3-yl)- | | | | |
| | 1H-1,2,4-triazol-3-yl)-5- | | | | |
| | (trifluoromethyl)tetrahydrofuran- | | | | |
| | 2-carboxamide | | | | |
| 562 | (2R,3S,4S,5R)-3-(3,4- | 519.462 | 520.26 | 3.04 | |
| | difluoro-2-(2- | | | | |
| | hydroxyethoxy)phenyl)- | | | | |
| | N-(4-(methoxymethyl)- | | | | |
| | 6-methylpyrimidin-2- | | | | |
| | yl)-4,5-dimethyl-5- | | | | |
| | (trifluoromethyl)tetrahydrofuran- | | | | |
| | 2-carboxamide | | | | |
| 563 | (2R,3S,4S,5R)-N-(1- | 557.513 | 558.27 | 3.62 | |
| | (tert-butyl)-1H- | | | | |
| | pyrazolo[3,4- | | | | |
| | d]pyrimidin-4-yl)-3- | | | | |
| | (3,4-difluoro-2-(2- | | | | |
| | hydroxyethoxy)phenyl)- | | | | |
| | 4,5-dimethyl-5- | | | | |
| | (trifluoromethyl)tetrahydrofuran- | | | | |
| | 2-carboxamide | | | | |
| 564 | (2R,3S,4S,5R)-3-(3,4- | 504.447 | 505.25 | 3 | |
| | difluoro-2-(2- | | | | |
| | hydroxyethoxy)phenyl)- | | | | |
| | N-(2-(methoxymethyl)pyridin- | | | | |
| | 4-yl)-4,5-dimethyl-5- | | | | |
| | (trifluoromethyl)tetrahydrofuran- | | | | |
| | 2-carboxamide | | | | |
| 565 | (2R,3S,4S,5R)-3-(3,4- | 515.433 | 516.31 | 2.91 | ¹H NMR (500 MHz, DMSO- |
| | difluoro-2-(2- | | | | d₆) δ 10.75 (s, 1H), 8.75 (d, J = |
| | hydroxyethoxy)phenyl)- | | | | 2.3 Hz, 1H), 8.71 (d, J = |
| | 4,5-dimethyl-N-(1- | | | | 2.3 Hz, 1H), 7.23-7.12 (m, |
| | methyl-1H- | | | | 2H), 5.19 (d, J = 10.8 Hz, |
| | [1,2,3]triazolo[4,5- | | | | 1H), 4.45 (dd, J = 10.7, 7.2 |
| | b]pyridin-6-yl)-5- | | | | Hz, 1H), 4.27 (s, 3H), 4.17- |
| | (trifluoromethyl)tetrahydrofuran- | | | | 4.07 (m, 2H), 3.73-3.70 (m, |
| | 2-carboxamide | | | | 2H), 2.95 (p, J = 7.4 Hz, 1H), |
| | | | | | 1.63 (s, 3H), 0.74 (d, J = 7.4 |
| | | | | | Hz, 3H) ppm. |
| 566 | (2R,3S,4S,5R)-3-(3,4- | 510.402 | 510.9 | 3.2 | |
| | difluoro-2-(2- | | | | |
| | hydroxyethoxy)phenyl)- | | | | |
| | N-(2-(difluoromethyl)pyridin- | | | | |
| | 4-yl)-4,5-dimethyl-5- | | | | |
| | (trifluoromethyl)tetrahydrofuran- | | | | |
| | 2-carboxamide | | | | |
| 567 | (2R,3S,4S,5R)-3-(3,4- | 534.473 | 535.26 | 3.14 | |
| | difluoro-2-(2- | | | | |
| | hydroxyethoxy)phenyl)- | | | | |
| | N-(2-(2-methoxyethoxy)pyridin- | | | | |
| | 4-yl)-4,5-dimethyl-5- | | | | |
| | (trifluoromethyl)tetrahydrofuran- | | | | |
| | 2-carboxamide | | | | |
| 568 | (2R,3S,4S,5R)-3-(3,4- | 491.408 | 492.3 | 2.84 | ¹H NMR (500 MHz, DMSO- |
| | difluoro-2-(2- | | | | d₆) δ 10.47 (s, 1H), 8.03 (d, J = |
| | hydroxyethoxy)phenyl)- | | | | 2.4 Hz, 1H), 7.25-7.07 |
| | 4,5-dimethyl-N-(1- | | | | (m, 3H), 5.10 (d, J = 10.5 Hz, |
| | methyl-6-oxo-1,6- | | | | 1H), 4.39 (dd, J = 10.6, 7.4 |
| | dihydropyridazin-4-yl)-5- | | | | Hz, 1H), 4.18-4.04 (m, 2H), |
| | (trifluoromethyl)tetrahydrofuran- | | | | 3.69 (t, J = 4.6 Hz, 2H), 3.57 |
| | 2-carboxamide | | | | (s, 3H), 2.89 (p, J = 7.2 Hz, |
| | | | | | 1H), 1.60 (s, 3H), 0.70 (d, J = |
| | | | | | 6.6 Hz, 3H) ppm. |
| 569 | (2R,3S,4S,5R)-3-(3,4- | 475.409 | 476.64 | 2.83 | ¹H NMR (500 MHz, DMSO- |
| | difluoro-2-(2- | | | | d₆) δ 10.32 (s, 1H), 8.88 (s, |
| | hydroxyethoxy)phenyl)- | | | | 2H), 7.22-7.10 (m, 2H), |
| | 4,5-dimethyl-N-(2- | | | | 5.12 (d, J = 10.8 Hz, 1H), |
| | methylpyrimidin-5-yl)-5- | | | | 4.40 (dd, J = 10.8, 7.3 Hz, |
| | (trifluoromethyl)tetrahydrofuran- | | | | 1H), 4.17-4.11 (m, 1H), |
| | 2-carboxamide | | | | 4.11-4.04 (m, 1H), 3.70- |
| | | | | | 3.68 (m, 2H), 2.89 (p, J = 7.4 |
| | | | | | Hz, 1H), 2.56 (s, 3H), 1.61 |
| | | | | | (s, 3H), 0.77-0.65 (m, 3H) |
| | | | | | ppm. |
| 570 | (2R,3S,4S,5R)-3-(3,4- | 508.411 | 509.3 | 3.39 | |
| | difluoro-2-(2- | | | | |
| | hydroxyethoxy)phenyl)- | | | | |
| | N-(5-fluoro-2- | | | | |
| | methoxypyridin-4-yl)- | | | | |
| | 4,5-dimethyl-5- | | | | |
| | (trifluoromethyl)tetrahydrofuran- | | | | |
| | 2-carboxamide | | | | |
| 571 | (2R,3S,4S,5R)-3-(3,4- | 510.402 | 511.2 | 3.21 | ¹H NMR (500 MHz, DMSO- |
| | difluoro-2-(2- | | | | d₆) δ 10.54 (s, 1H), 8.85 (s, |
| | hydroxyethoxy)phenyl)- | | | | 1H), 8.23 (d, J = 8.8 Hz, 1H), |
| | N-(6-(difluoromethyl)pyridin- | | | | 7.65 (d, J = 8.5 Hz, 1H), 7.22- |
| | 3-yl)-4,5-dimethyl-5- | | | | 7.10 (m, 2H), 6.89 (t, J = |
| | (trifluoromethyl)tetrahydrofuran- | | | | 55.1 Hz, 1H), 5.12 (d, J = |
| | 2-carboxamide | | | | 10.9 Hz, 1H), 4.97 (s, 1H), |
| | | | | | 4.41 (dd, J = 10.7, 7.3 Hz, |
| | | | | | 1H), 4.19-4.03 (m, 2H), |
| | | | | | 3.70 (s, 2H), 2.91 (t, J = 7.4 |
| | | | | | Hz, 1H), 1.61 (s, 3H), 0.72 |
| | | | | | (d, J = 7.5 Hz, 3H) ppm. |
| 572 | (2R,3S,4S,5R)-3-(3,4- | 466.422 | 466.8 | 3.24 | ¹H NMR (500 MHz, DMSO- |
| | difluoro-2-(2- | | | | d₆) δ 10.77 (s, 1H), 8.94 (s, |
| | hydroxyethoxy)phenyl)- | | | | 1H), 8.69 (s, 1H), 7.24-7.08 |
| | N-(isothiazol-4-yl)-4,5- | | | | (m, 2H), 5.11 (d, J = 10.8 Hz, |
| | dimethyl-5- | | | | 1H), 4.96 (s, 1H), 4.37 (dd, J = |
| | (trifluoromethyl)tetrahydrofuran- | | | | 10.9, 7.3 Hz, 1H), 4.15- |
| | 2-carboxamide | | | | 4.04 (m, 2H), 3.70 (t, J = 4.6 |
| | | | | | Hz, 2H), 2.90 (p, J = 7.5 Hz, |
| | | | | | 1H), 1.61 (s, 3H), 0.79-0.66 |
| | | | | | (m, 3H) ppm. |
| 573 | (2R,3S,4S,5R)-3-(3,4- | 464.386 | 465.2 | 2.94 | ¹H NMR (500 MHz, DMSO- |
| | difluoro-2-(2- | | | | d₆) δ 11.11 (s, 1H), 7.80 (s, |
| | hydroxyethoxy)phenyl)- | | | | 1H), 7.23-7.11 (m, 2H), |
| | 4,5-dimethyl-N-(1- | | | | 5.10 (d, J = 10.9 Hz, 1H), |
| | methyl-1H-1,2,3-triazol-5-yl)-5- | | | | 5.04-4.83 (m, 1H), 4.35 (dd, |
| | (trifluoromethyl)tetrahydrofuran- | | | | J = 10.9, 7.2 Hz, 1H), 4.10 |
| | 2-carboxamide | | | | (q, J = 4.0, 3.5 Hz, 2H), 4.05 |
| | | | | | (s, 3H), 3.71 (t, J = 4.7 Hz, |
| | | | | | 2H), 2.92 (p, J = 7.4 Hz, 1H), |
| | | | | | 1.58 (s, 3H), 0.69 (d, J = 7.4 |
| | | | | | Hz, 3H) ppm. |
| 574 | (2R,3S,4S,5R)-3-(3,4- | 514.445 | 515.2 | 3.01 | ¹H NMR (500 MHz, DMSO- |
| | difluoro-2-(2- | | | | d₆) δ 10.55 (s, 1H), 8.89 (dd, |
| | hydroxyethoxy)phenyl)- | | | | J = 7.5, 0.9 Hz, 1H), 8.25 (d, |
| | 4,5-dimethyl-N-(3-methyl- | | | | J = 2.0 Hz, 1H), 7.17 (t, J = |
| | [1,2,3]triazolo[1,5- | | | | 7.2, 6.7, 2.3 Hz, 3H), 5.12 (d, |
| | a]pyridin-5-yl)-5- | | | | J = 10.7 Hz, 1H), 4.43 (dd, J = |
| | (trifluoromethyl)tetrahydrofuran- | | | | 10.7, 7.2 Hz, 1H), 4.19- |
| | 2-carboxamide | | | | 4.06 (m, 2H), 3.75-3.70 (m, |
| | | | | | 2H), 2.93 (p, J = 7.5 Hz, 1H), |
| | | | | | 2.45 (s, 3H), 1.61 (s, 3H), |
| | | | | | 0.75-0.69 (m, 3H) ppm. |

The following compound was made using a method similar to that described in Example 16, except that amide coupling conditions similar to Example 7, step 8 with pyridin-3-amine were used in step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 575 | (2R,3S,4S,5R)-3-(3,4- | 460.394 | 461.7 | 2.93 | ¹H NMR (500 MHz, DMSO- |
| | difluoro-2-(2- | | | | d₆) δ 10.37 (s, 1H), 8.76 (dd, |
| | hydroxyethoxy)phenyl)- | | | | J = 2.7, 0.8 Hz, 1H), 8.30 |
| | 4,5-dimethyl-N- | | | | (dd, J = 4.7, 1.5 Hz, 1H), |
| | (pyridin-3-yl)-5- | | | | 8.03 (ddd, J = 8.3, 2.6, 1.5 |
| | (trifluoromethyl)tetra- | | | | Hz, 1H), 7.35 (ddd, J = 8.4, |
| | hydrofuran-2-carboxamide | | | | 4.7, 0.8 Hz, 1H), 7.17 (dd, |
| | | | | | J = 8.5, 5.1 Hz, 2H), 5.11 (d, |
| | | | | | J = 10.7 Hz, 1H), 4.40 (dd, J = |
| | | | | | 10.7, 7.2 Hz, 1H), 4.18-4.04 |
| | | | | | (m, 2H), 3.71 (d, J = 5.1 Hz, |
| | | | | | 2H), 2.92 (p, J = 7.4 Hz, 1H), |
| | | | | | 1.61 (s, 3H), 0.94 (t, J = 7.4 |
| | | | | | Hz, 1H), 0.78-0.65 (m, 3H) ppm. |

The following compound was made using a method similar to that described in Example 16, except that alternative amide coupling conditions were used in step 3. For step 3, a solution of (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylic acid (175 mg, 0.3159 mmol), pyridazin-4-amine (33 mg, 0.3470 mmol), 1-methylimidazole (90 μL, 1.129 mmol), and TCFH (105 mg, 0.3742 mmol) in MeCN (5 mL) were stirred overnight at ambient temperature. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 576 | (2R,3S,4S,5R)-3-(3,4- | 461.383 | 462 | 2.81 | ¹H NMR (500 MHz, DMSO- |
| | difluoro-2-(2- | | | | d₆) δ 10.66 (s, 1H), 9.38 (d, |
| | hydroxyethoxy)phenyl)- | | | | J = 2.6 Hz, 1H), 9.04 (d, J = |
| | 4,5-dimethyl-N- | | | | 5.9 Hz, 1H), 7.95 (dd, J = |
| | (pyridazin-4-yl)-5- | | | | 5.9, 2.7 Hz, 1H), 7.20-7.13 |
| | (trifluoromethyl)tetra- | | | | (m, 2H), 5.16 (d, J = 10.6 Hz, |
| | hydrofuran-2-carboxamide | | | | 1H), 4.96 (s, 1H), 4.42 (dd, |
| | | | | | J = 10.6, 7.4 Hz, 1H), 4.16- |
| | | | | | 4.07 (m, 2H), 3.77-3.64 (m, |
| | | | | | 2H), 2.91 (p, J = 7.5 Hz, 1H), |
| | | | | | 1.62 (s, 3H), 0.72 (d, J = 8.4 |
| | | | | | Hz, 3H) ppm. |

The following compound was made using a method similar to that described in Example 16, except that [1,2,4]triazolo[4,3-a]pyridin-6-amine was used in amide coupling step 3 and O-alkylation using methyl iodide and sodium hydride in THF was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 577 | (2R,3S,4S,5R)-N- | 514.445 | 514.9 | 3.28 | ¹H NMR (500 MHz, DMSO- |
| | ([1,2,4]triazolo[4,3- | | | | d₆) δ 10.44 (s, 1H), 9.29- |
| | a]pyridin-6-yl)-3-(3,4- | | | | 9.25 (m, 1H), 9.22 (s, 1H), |
| | difluoro-2-(2- | | | | 7.76 (d, J = 9.8 Hz, 1H), 7.38 |
| | methoxyethoxy)phenyl)- | | | | (dd, J = 9.8, 1.9 Hz, 1H), |
| | 4,5-dimethyl-5- | | | | 7.19-7.12 (m, 2H), 5.12 (d, |
| | (trifluoromethyl)tetra- | | | | J = 10.8 Hz, 1H), 4.37 (dd, |
| | hydrofuran-2-carboxamide | | | | J = 10.8, 7.2 Hz, 1H), 4.33- |
| | | | | | 4.25 (m, 1H), 4.23-4.14 (m, |
| | | | | | 1H), 3.68-3.57 (m, 2H), |
| | | | | | 3.28 (s, 3H), 2.84 (p, J = 7.5 |
| | | | | | Hz, 1H), 1.62 (s, 3H), 0.72 |
| | | | | | (d, J = 7.3 Hz, 3H) ppm. |

The following compounds were made using a method similar to that described in Example 16, except that 3-(bromomethyl)oxetane was used as the alkylating agent in step 1, different amines were used in amide coupling step 3 and deprotection step 4 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 578 | (2R,3S,4S,5R)-3-(3,4- | 486.432 | 487.2 | 3.11 | ¹H NMR (400 MHz, |
| | difluoro-2-(oxetan-3- | | | | Methanol-d₄) δ 8.77-8.73 |
| | ylmethoxy)phenyl)-4,5- | | | | (m, 1H), 8.28 (dd, J = 4.9, |
| | dimethyl-N-(pyridin-3- | | | | 1.5 Hz, 1H), 8.10 (ddd, J = |
| | yl)-5- | | | | 8.4, 2.6, 1.5 Hz, 1H), 7.39 |
| | (trifluoromethyl)tetra- | | | | (ddd, J = 8.4, 4.8, 0.8 Hz, |
| | hydrofuran-2-carboxamide | | | | 1H), 7.17 (ddd, J = 8.2, 5.6, |
| | | | | | 2.2 Hz, 1H), 7.06-6.95 (m, |
| | | | | | 1H), 5.09 (d, J = 10.5 Hz, |
| | | | | | 1H), 4.89-4.85 (m, 2H), |
| | | | | | 4.63 (dt, J = 9.8, 6.1 Hz, 2H), |
| | | | | | 4.50 (ddd, J = 10.0, 5.9, 2.0 |
| | | | | | Hz, 1H), 4.37 (dd, J = 10.6, |
| | | | | | 7.9 Hz, 1H), 4.30 (ddd, J = |
| | | | | | 10.1, 5.9, 1.6 Hz, 1H), 3.44 |
| | | | | | (ttd, J = 8.0, 5.9, 5.3, 2.3 Hz, |
| | | | | | 1H), 2.79 (p, J = 7.6 Hz, 1H), |
| | | | | | 1.66 (d, J = 1.3 Hz, 3H), 0.82 |
| | | | | | (dq, J = 7.5, 2.4 Hz, 3H) ppm. |
| 579 | (2R,3S,4S,5R)-3-(3,4- | 539.443 | 540.2 | 3.34 | ¹H NMR (400 MHz, |
| | difluoro-2-(oxetan-3- | | | | Methanol-d₄) δ 8.19 (s, 1H), |
| | ylmethoxy)phenyl)-N- | | | | 7.30 (t, J = 59.8 Hz, 1H), |
| | (1-(difluoromethyl)-3- | | | | 7.21-7.11 (m, 1H), 7.01 (td, |
| | methyl-1H-pyrazol-4- | | | | J = 9.4, 7.6 Hz, 1H), 5.12 (d, |
| | yl)-4,5-dimethyl-5- | | | | J = 10.6 Hz, 1H), 4.87 (ddd, |
| | (trifluoromethyl)tetra- | | | | J = 8.0, 6.2, 2.6 Hz, 2H), 4.63 |
| | hydrofuran-2-carboxamide | | | | (dt, J = 12.1, 6.1 Hz, 2H), |
| | | | | | 4.49 (ddd, J = 10.0, 6.0, 1.9 |
| | | | | | Hz, 1H), 4.35 (dd, J = 10.7, |
| | | | | | 7.8 Hz, 1H), 4.29 (ddd, J = |
| | | | | | 10.0, 5.9, 1.6 Hz, 1H), 3.44 |
| | | | | | (tt, J = 7.8, 5.7 Hz, 1H), 2.78 |
| | | | | | (p, J = 7.6 Hz, 1H), 2.20 (s, |
| | | | | | 3H), 1.65 (d, J = 1.1 Hz, 3H), |
| | | | | | 0.82 (dq, J = 7.4, 2.3 Hz, 3H) ppm. |
| 580 | 5-((2R,3S,4S,5R)-3-(3,4- | 529.456 | 530.2 | 3.04 | ¹H NMR (400 MHz, |
| | difluoro-2-(oxetan-3- | | | | Methanol-d₄) δ 8.85 (dd, J = |
| | ylmethoxy)phenyl)-4,5- | | | | 2.5, 0.7 Hz, 1H), 8.20 (dd, |
| | dimethyl-5- | | | | J = 8.6, 2.5 Hz, 1H), 8.05 (dd, |
| | (trifluoromethyl)tetra- | | | | J = 8.6, 0.7 Hz, 1H), 7.17 |
| | hydrofuran-2- | | | | (ddd, J = 8.3, 5.6, 2.2 Hz, |
| | carboxamido)picolinamide | | | | 1H), 7.01 (td, J = 9.4, 7.6 Hz, |
| | | | | | 1H), 5.11 (d, J = 10.5 Hz, |
| | | | | | 1H), 4.91-4.85 (m, 2H), |
| | | | | | 4.64 (dt, J = 11.2, 6.0 Hz, |
| | | | | | 2H), 4.51 (ddd, J = 10.0, 6.0, |
| | | | | | 1.9 Hz, 1H), 4.39 (dd, J = |
| | | | | | 10.5, 7.9 Hz, 1H), 4.31 (ddd, |
| | | | | | J = 10.0, 5.8, 1.5 Hz, 1H), |
| | | | | | 3.44 (ddd, J = 13.8, 7.8, 5.8 |
| | | | | | Hz, 1H), 2.80 (p, J = 7.6 Hz, |
| | | | | | 1H), 1.66 (d, J = 1.1 Hz, 3H), |
| | | | | | 0.83 (dt, J = 7.7, 2.3 Hz, 3H) ppm. |

The following compound was made using a method similar to that described in Example 16, except that 3-(bromomethyl)oxetane was used as the alkylating agent in step 1 and methyl 5-aminopyridine-2-carboxylate was used as the amine in step 3. Step 4 was omitted and the product of step 3 was treated with an excess of methyl magnesium bromide in THF as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 581 | (2R,3S,4S,5R)-3-(3,4- | 544.511 | 545.2 | 3.15 | ¹H NMR (400 MHz, |
| | difluoro-2-(oxetan-3- | | | | Methanol-d₄) δ 9.08 (d, J = |
| | ylmethoxy)phenyl)-N- | | | | 2.3 Hz, 1H), 8.42 (dd, J = |
| | (6-(2-hydroxypropan-2- | | | | 8.9, 2.5 Hz, 1H), 7.93 (dd, |
| | yl)pyridin-3-yl)-4,5- | | | | J = 8.9, 0.7 Hz, 1H), 7.18 (ddd, |
| | dimethyl-5- | | | | J = 8.2, 5.5, 2.1 Hz, 1H), |
| | (trifluoromethyl)tetra- | | | | 7.01 (td, J = 9.3, 7.5 Hz, 1H), |
| | hydrofuran-2-carboxamide | | | | 5.13 (d, J = 10.6 Hz, 1H), |
| | (TFA salt) | | | | 4.90-4.84 (m, 2H), 4.62 (dt, |
| | | | | | J = 19.4, 6.1 Hz, 2H), 4.50 |
| | | | | | (ddd, J = 10.0, 5.8, 1.9 Hz, |
| | | | | | 1H), 4.39 (dd, J = 10.6, 8.0 |
| | | | | | Hz, 1H), 4.32 (ddd, J = 10.1, |
| | | | | | 5.9, 1.6 Hz, 1H), 3.44 (ddd, |
| | | | | | J = 13.9, 7.9, 5.8 Hz, 1H), 2.80 |
| | | | | | (p, J = 7.6 Hz, 1H), 1.66 (d, |
| | | | | | J = 1.1 Hz, 3H), 1.60 (s, 6H), |
| | | | | | 0.83 (dd, J = 7.6, 2.4 Hz, 3H) ppm. |

The following compounds were made using methods similar to those described in Example 16, except that 1-bromo-2-methoxyethane was used as the alkylating agent in step 1, t-BuOK in THF was used as base in step 2 and different amines were used in step 3 using similar amide coupling conditions to those described in Example 7, step 8. Step 4 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 582 | (2R,3S,4S,5R)-3-(3,4- | 535.504 | 534.3 | 3.24 | ¹H NMR (500 MHz, DMSO- |
| | difluoro-2-(2- | | | | d₆) δ 10.17 (s, 1H), 7.95 (d, |
| | methoxyethoxy)phenyl)- | | | | J = 0.7 Hz, 1H), 7.52 (d, J = |
| | N-(1-(1-hydroxy-2- | | | | 0.7 Hz, 1H), 7.20-7.11 (m, |
| | methylpropan-2-yl)-1H- | | | | 2H), 5.03 (d, J = 11.0 Hz, |
| | pyrazol-4-yl)-4,5- | | | | 1H), 4.92 (t, J = 5.6 Hz, 1H), |
| | dimethyl-5- | | | | 4.33-4.16 (m, 3H), 3.66- |
| | (trifluoromethyl)tetra- | | | | 3.58 (m, 2H), 3.52 (d, J = 5.6 |
| | hydrofuran-2-carboxamide | | | | Hz, 2H), 3.28 (s, 3H), 2.81 (t, |
| | | | | | J = 7.3 Hz, 1H), 1.59 (s, 3H), |
| | | | | | 1.41 (s, 6H), 0.69 (d, J = 7.4 |
| | | | | | Hz, 3H) ppm. |
| 583 | (2R,3S,4S,5R)-3-(3,4- | 535.504 | 536.3 | 3.21 | ¹H NMR (500 MHz, DMSO- |
| | difluoro-2-(2- | | | | d₆) δ 10.21 (s, 1H), 7.92 (d, |
| | methoxyethoxy)phenyl)- | | | | J = 0.7 Hz, 1H), 7.50 (d, J = |
| | N-(1-(2-hydroxy-2- | | | | 0.7 Hz, 1H), 7.19-7.09 (m, |
| | methylpropyl)-1H- | | | | 2H), 5.03 (d, J = 10.9 Hz, |
| | pyrazol-4-yl)-4,5- | | | | 1H), 4.63 (s, 1H), 4.37-4.11 |
| | dimethyl-5- | | | | (m, 3H), 3.93 (s, 2H), 3.61 |
| | (trifluoromethyl)tetra- | | | | (ddd, J = 5.4, 3.1, 2.0 Hz, |
| | hydrofuran-2-carboxamide | | | | 2H), 3.27 (s, 3H), 2.80 (p, J = |
| | | | | | 7.4 Hz, 1H), 1.60 (s, 3H), |
| | | | | | 1.02 (d, J = 1.6 Hz, 6H), 0.69 |
| | | | | | (dd, J = 7.6, 2.5 Hz, 3H) ppm. |
| 584 | (2R,3S,4S,5R)-3-(3,4- | 504.447 | 504.9 | 3.07 | |
| | difluoro-2-(2- | | | | |
| | methoxyethoxy)phenyl)- | | | | |
| | 4,5-dimethyl-N-(1- | | | | |
| | methyl-6-oxo-1,6- | | | | |
| | dihydropyridin-3-yl)-5- | | | | |
| | (trifluoromethyl)tetra- | | | | |
| | hydrofuran-2-carboxamide | | | | |

The following compound was made using methods similar to those described in Example 16, except that 1-bromo-2-methoxyethane was used as the alkylating agent in step 1, KO-t-Bu in THF was used as base in step 2 and conditions described in Example 10 were used as the final steps, in place of step 3 and 4 above. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 585 | (2R,3S,4S,5R)-3-(3,4- | 586.551 | 587.1 | 3.21 | ¹H NMR (500 MHz, DMSO- |
| | difluoro-2-(2- | | | | d₆) δ 10.65 (s, 1H), 8.31 (dd, |
| | methoxyethoxy)phenyl)- | | | | J = 5.6, 0.6 Hz, 1H), 8.11 |
| | 4,5-dimethyl-N-(2-(4- | | | | (dd, J = 2.0, 0.6 Hz, 1H), |
| | methyl-2-oxopiperazin- | | | | 7.54 (dd, J = 5.6, 1.9 Hz, |
| | 1-yl)pyridin-4-yl)-5- | | | | 1H), 7.25-7.06 (m, 2H), |
| | (trifluoromethyl)tetra- | | | | 5.11 (d, J = 10.7 Hz, 1H), |
| | hydrofuran-2-carboxamide | | | | 4.35 (dd, J = 10.7, 7.2 Hz, |
| | | | | | 1H), 4.31-4.25 (m, 1H), |
| | | | | | 4.20 (dddd, J = 11.1, 4.8, 3.2, |
| | | | | | 1.3 Hz, 1H), 3.83 (td, J = 5.5, |
| | | | | | 4.2 Hz, 2H), 3.68-3.56 (m, |
| | | | | | 2H), 3.29 (s, 3H), 3.16 (s, |
| | | | | | 2H), 2.84 (p, J = 7.5 Hz, 1H), |
| | | | | | 2.71 (t, J = 5.5 Hz, 2H), 2.27 |
| | | | | | (s, 3H), 1.60 (s, 3H), 0.75- |
| | | | | | 0.65 (m, 3H) ppm. |

### Example 17

(2R,3S,4S,5R)-3-(3,4-difluoro-2-(2-morpholinoethoxy)phenyl)-4,5-dimethyl-N-(3-methylisoxazol-4-yl)-5-(trifluoromethyl)tetrahydrofuran-2-carboxamide (586)

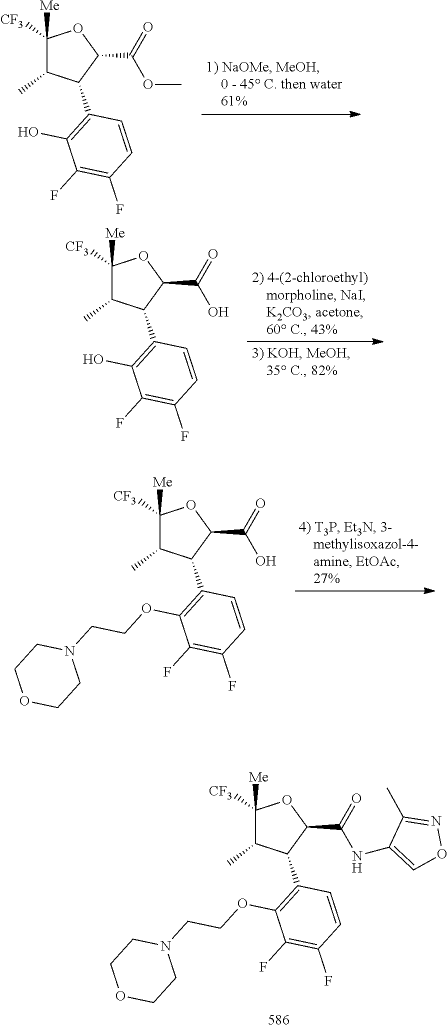

### Step 1:

To a solution of methyl (2S,3S,4S,5R)-3-(3,4-difluoro-2-hydroxy-phenyl)-4,5-dimethyl-5-(trifluoromethyl)tetrahydrofuran-2-carboxylate (5 g, 14.11 mmol) in MeOH (20 mL) at 0° C. was added sodium methoxide (9.14 mL of 25% w/v solution in MeOH, 42.30 mmol) dropwise over 10 minutes. The solution was stirred at 0° C. for 1 h, then heated at 45° C. overnight. Water was added (1.52 mL, 84.37 mmol) and the reaction mixture was heated at 45° C. for 1 h. The reaction mixture was concentrated in vacuo and partitioned between 2-MeTHF (50 mL) and water (25 mL). The aqueous layer was acidified to pH 1 with HCl and the layers were separated. The aqueous layer was extracted again with 2-MeTHF (10 mL). The combined organic extracts were dried (Na₂SO₄) and concentrated in vacuo to afford (2R,3S,4S,5R)-3-(3,4-difluoro-2-hydroxy-phenyl)-4,5-dimethyl-5-(trifluoromethyl)tetrahydrofuran-2-carboxylic acid (3.9 g, 61%) as a pale yellow oil. ESI-MS m/z calc. 340.0734, found 339.1 (M−1)⁻; Retention time: 0.46 minutes.

### Step 2:

To a solution of (2R,3S,4S,5R)-3-(3,4-difluoro-2-hydroxy-phenyl)-4,5-dimethyl-5-(trifluoromethyl)tetrahydrofuran-2-carboxylic acid (4 g, 11.76 mmol) in acetone (30 mL) was added 4-(2-chloroethyl)morpholine (HCl salt) (6.56 g, 35.25 mmol), NaI (1.76 g, 11.74 mmol) and K₂CO₃ (8.12 g, 58.75 mmol) and the mixture was heated to 60° C. for 24 hours. The reaction mixture was cooled to ambient temperature and partitioned between MTBE (100 mL) and water (100 mL). The aqueous layer was further extracted with MTBE (30 mL). The combined organic fractions were washed with brine (10 mL), dried (MgSO₄) and concentrated in vacuo. Purification by flash chromatography (SiO₂, 0 to 100% EtOAc in heptanes then 0 to 25% MeOH in DCM) gave 2-morpholinoethyl (2R,3S,4S,5R)-3-[3,4-difluoro-2-(2-morpholinoethoxy)phenyl]-4,5-dimethyl-5-(trifluoromethyl)tetrahydrofuran-2-carboxylate (4.8 g, 43%) as a light yellow oil. ESI-MS m/z calc. 566.2415, found 567.3 (M+1)⁺; Retention time: 0.96 minutes.

### Step 3:

To a solution of 2-morpholinoethyl (2R,3S,4S,5R)-3-[3,4-difluoro-2-(2-morpholinoethoxy)phenyl]-4,5-dimethyl-5-(trifluoromethyl)tetrahydrofuran-2-carboxylate (4 g, 7.060 mmol) in MeOH (20 mL) was added ground KOH (990 mg, 17.65 mmol) and the reaction mixture was heated at 35° C. for 90 min. Water (20 mL) was added and the MeOH was removed in vacuo. To the residue was added 1 M NaOH until pH 14 was obtained. The mixture was extracted with MTBE (20 mL) organic phase was extracted with water (×2). The combined aqueous phases were then acidified to pH 4.6 with 6 N HCl and extracted with EtOAc (×3). The combined organic extracts were dried (MgSO₄), filtered, and concentrated in vacuo to yield (2R,3S,4S,5R)-3-[3,4-difluoro-2-(2-morpholinoethoxy)phenyl]-4,5-dimethyl-5-(trifluoromethyl)tetrahydrofuran-2-carboxylic acid (2.7 g, 82%) as a white solid. ¹H NMR (500 MHz, DMSO-d₆) δ 7.19 (ddd, J=7.9, 5.8, 1.7 Hz, 1H), 7.12 (td, J=9.4, 7.5 Hz, 1H), 5.01 (d, J=10.7 Hz, 1H), 4.39-4.29 (m, 1H), 4.22 (ddd, J=17.1, 10.8, 6.0 Hz, 2H), 3.57 (t, J=4.7 Hz, 4H), 2.83-2.61 (m, 3H), 2.45 (s, 4H), 1.56 (s, 3H), 0.67 (dt, J=7.3, 2.3 Hz, 3H) ppm. ESI-MS m/z calc. 453.15747, found 454.4 (M+1)⁺; 452.2 (M−1)⁻; Retention time: 0.55 minutes.

### Step 4:

To a solution of (2R,3S,4S,5R)-3-[3,4-difluoro-2-(2-morpholinoethoxy)phenyl]-4,5-dimethyl-5-(trifluoromethyl)tetrahydrofuran-2-carboxylic acid (60 mg, 0.128 mmol) in EtOAc (2 mL) was added 3-methylisoxazol-4-amine (25 mg, 0.254 mmol), Et₃N (53 μL, 0.38 mmol) and T3P solution (120 μL of 50% w/v in EtOAc, 0.403 mmol). The reaction mixture was stirred at ambient temperature for 4 hours. The reaction mixture was partitioned between EtOAc (10 mL) and water (10 mL) and the organic layer was washed with water (5 mL). The organic fraction was washed with brine (10 mL), dried (MgSO₄), and concentrated in vacuo. Purification by preparative reverse phase HPLC-MS using an X-bridge C18 column (150×19 mm, 5 μm particle size) from Waters (basic eluent) gave (2R,3S,4S,5R)-3-(3,4-difluoro-2-(2-morpholinoethoxy)phenyl)-4,5-dimethyl-N-(3-methylisoxazol-4-yl)-5-(trifluoromethyl)tetrahydrofuran-2-carboxamide (586, 19 mg, 27) as a white solid. ¹H NMR (500 MHz, DMSO-d₆) δ 10.06 (s, 1H), 9.03 (s, 1H), 7.14 (dd, d=8.5, 5.0 Hz, 2H), 5.21 (d, J=10.7 Hz, 1H), 4.31 (dd, J=10.9, 6.8 Hz, 2H), 4.23 (ddt, J=11.1, 5.5, 2.8 Hz, 1H), 3.54 (td, J=4.5, 1.8 Hz, 4H), 2.91 (p, =7.4 Hz, 1H), 2.66 (t, i=5.2 Hz, 2H), 2.41 (t, J=4.7 Hz, 4H), 2.24 (s, 3H), 1.61 (s, 3H), 0.74-0.68 (, 3H) ppm. ESI-MS m/z calc. 533.1949, found 534.2 (M+1; Retention time: 3.28 minutes.

The following compounds were made using a method similar to that described in Example 17, except that different amines were used in amide coupling step 4. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 587 | (2R,3S,4S,5R)-3-(3,4- | 529.5 | 530.2 | 3.18 | ¹H NMR (500 MHz, DMSO- |
| | difluoro-2-(2- | | | | d₆) δ 10.38 (s, 1H), 8.77 (d, J = |
| | morpholinoethoxy)phenyl)- | | | | 2.5 Hz, 1H), 8.29 (dd, J = 4.7, |
| | 4,5-dimethyl-N- | | | | 1.4 Hz, 1H), 8.04 (ddd, J = 8.4, |
| | (pyridin-3-yl)-5- | | | | 2.7, 1.5 Hz, 1H), 7.35 (dd, J = |
| | (trifluoromethyl)tetra- | | | | 8.3, 4.7 Hz, 1H), 7.21-7.09 |
| | hydrofuran-2-carboxamide | | | | (m, 2H), 5.13 (d, J = 10.6 Hz, |
| | | | | | 1H), 4.38-4.28 (m, 2H), 4.26- |
| | | | | | 4.18 (m, 1H), 3.57-3.45 (m, |
| | | | | | 4H), 2.88 (p, J = 7.5 Hz, 1H), |
| | | | | | 2.65 (dd, J = 6.1, 4.5 Hz, 2H), |
| | | | | | 2.40 (t, J = 4.6 Hz, 4H), 1.64 |
| | | | | | (s, 3H), 0.74-0.68 (m, 3H) ppm. |
| 588 | (2R,3S,4S,5R)-3-(3,4- | 559.526 | 560.3 | 2.99 | ¹H NMR (500 MHz, DMSO- |
| | difluoro-2-(2- | | | | d₆) δ 10.26 (s, 1H), 7.60 (d, J = |
| | morpholinoethoxy)phen | | | | 7.4 Hz, 1H), 7.19-7.07 (m, |
| | yl)-4,5-dimethyl-N-(1- | | | | 2H), 6.76 (d, J = 2.3 Hz, 1H), |
| | methyl-2-oxo-1,2- | | | | 6.41 (dd, J = 7.4, 2.4 Hz, 1H), |
| | dihydropyridin-4-yl)-5- | | | | 5.07 (d, J = 10.4 Hz, 1H), 4.36- |
| | (trifluoromethyl)tetra- | | | | 4.28 (m, 2H), 4.22 (dt, J = |
| | hydrofuran-2-carboxamide | | | | 11.4, 4.9 Hz, 1H), 3.53 (q, J = |
| | | | | | 4.5 Hz, 4H), 3.34 (s, 3H), 2.87 |
| | | | | | (p, J = 7.4 Hz, 1H), 2.65 (dd, |
| | | | | | J = 6.1, 4.6 Hz, 2H), 2.41 (t, J = |
| | | | | | 4.7 Hz, 4H), 1.61 (s, 3H), 0.72- |
| | | | | | 0.67 (m, 3H) ppm. |
| 589 | (2R,3S,4S,5R)-3-(3,4- | 559.526 | 560 | 3.24 | ¹H NMR (500 MHz, DMSO- |
| | difluoro-2-(2- | | | | d₆) δ 10.55 (s, 1H), 8.32 (d, J = |
| | morpholinoethoxy)phen | | | | 5.6 Hz, 1H), 7.70 (d, J = 2.0 |
| | yl)-N-(2- | | | | Hz, 1H), 7.50 (dd, J = 5.6, 2.1 |
| | (hydroxymethyl)pyridin | | | | Hz, 1H), 7.18-7.11 (m, 2H), |
| | 4-yl)-4,5-dimethyl-5-- | | | | 5.38 (s, 1H), 5.11 (d, J = 10.5 |
| | (trifluoromethyl)tetra- | | | | Hz, 1H), 4.49 (s, 2H), 4.37- |
| | hydrofuran-2-carboxamide | | | | 4.28 (m, 2H), 4.22 (ddt, J = |
| | | | | | 10.7, 5.3, 2.8 Hz, 1H), 3.52 (h, |
| | | | | | J = 6.7 Hz, 4H), 2.88 (p, J = |
| | | | | | 7.4 Hz, 1H), 2.66 (t, J = 5.2 |
| | | | | | Hz, 2H), 2.40 (t, J = 4.9 Hz, |
| | | | | | 4H), 1.63 (s, 3H), 0.71 (d, J = |
| | | | | | 7.3 Hz, 3H) ppm. |
| 590 | (2R,3S,4S,5R)-N- | 569.524 | 570.3 | 3 | ¹H NMR (500 MHz, DMSO- |
| | ([1,2,4]triazolo[4,3- | | | | d₆) δ 10.45 (s, 1H),9.28-9.22 |
| | alpyridin-6-yl)-3-(3,4- | | | | (m, 2H), 7.77 (dd, J = 9.9, 1.0 |
| | difluoro-2-(2- | | | | Hz, 1H), 7.41 (dd, J = 9.8, 1.9 |
| | morpholinoethoxy)phenyl)- | | | | Hz, 1H), 7.21-7.10 (m, 2H), |
| | 4,5-dimethyl-5- | | | | 5.15 (d, J = 10.6 Hz, 1H), 4.34 |
| | (trifluoromethyl)tetra- | | | | (ddd, J = 15.7, 11.0, 6.6 Hz, |
| | hydrofuran-2-carboxamide | | | | 2H), 4.22 (ddt, J = 9.6, 5.9, 2.9 |
| | | | | | Hz, 1H), 3.51 (q, J = 4.9 Hz, |
| | | | | | 4H), 2.89 (p, J = 7.4 Hz, 1H), |
| | | | | | 2.66 (t, J = 5.2 Hz, 2H), 2.40 |
| | | | | | (t, J = 4.7 Hz, 4H), 1.65 (s, |
| | | | | | 3H), 0.75-0.69 (m, 3H) ppm. |
| 591 | (2R,3S,4S,5R)-3-(3,4- | 582.511 | 583.3 | 3.41 | ¹H NMR (500 MHz, DMSO- |
| | difluoro-2-(2- | | | | d₆) δ 9.97 (s, 1H), 8.30 (s, 1H), |
| | morpholinoethoxy)phenyl)- | | | | 7.63 (t, J = 59 Hz, 1H), 7.20- |
| | N-(1-(difluoromethyl)- | | | | 7.11 (m, 2H), 5.20 (d, J = 10.7 |
| | 3-methyl-1H-pyrazol-4- | | | | Hz, 1H), 4.35-4.27 (m, 2H), |
| | yl)-4,5-dimethyl-5- | | | | 4.22 (ddd, J = 10.8, 5.5, 3.8 |
| | (trifluoromethyl)tetra- | | | | Hz, 1H), 3.53 (td, J = 4.4, 2.0 |
| | hydrofuran-2-carboxamide | | | | Hz, 4H), 2.90 (q, J = 7.5 Hz, |
| | | | | | 1H), 2.66 (t, J = 5.2 Hz, 2H), |
| | | | | | 2.41 (t, J = 4.6 Hz, 4H), 2.18 |
| | | | | | (s, 3H), 1.61 (s, 3H), 0.76- |
| | | | | | 0.67 (m, 3H) ppm. |
| 57 | (2R,3S,4S,5R)-3-(3,4- | 610.594 | 611.3 | 3.3 | ¹H NMR (500 MHz, DMSO- |
| | difluoro-2-(2- | | | | d₆) δ 10.12 (s, 1H), 8.34 (s, |
| | morpholinoethoxy)phenyl)- | | | | 1H), 7.18-7.11 (m, 2H), 5.22 |
| | 4,5-dimethyl-N-(3- | | | | (d, J = 10.6 Hz, 1H), 4.33 (dd, |
| | methyl-1- | | | | J = 10.8, 7.0 Hz, 2H), 4.27- |
| | (methylsulfonyl)-1H- | | | | 4.20 (m, 1H), 3.54 (td, J = 4.4, |
| | pyrazol-4-yl)-5- | | | | 1.9 Hz, 4H), 3.43 (s, 3H), 2.91 |
| | (trifluoromethyl)tetrahy | | | | (p, J = 7.5 Hz, 1H), 2.67 (t, J = |
| | drofuran-2-carboxamide | | | | 5.2 Hz, 2H), 2.45 (d, J = 18.8 |
| | | | | | Hz, 2H), 2.42 (s, 2H), 2.25 (s, |
| | | | | | 3H), 1.61 (s, 3H), 0.72 (d, J = |
| | | | | | 7.2 Hz, 3H) ppm. |
| 592 | 5-((2R,3S,4S,5R)-3-(3,4- | 575.528 | 576.3 | 2.92 | ¹H NMR (500 MHz, DMSO- |
| | difluoro-2-(2- | | | | d₆) δ 10.31 (s, 1H), 7.40 (s, |
| | morpholinoethoxy)phenyl)- | | | | 1H), 7.23-7.11 (m, 3H), 6.53 |
| | 4,5-dimethyl-5- | | | | (s, 1H), 5.20 (d, J = 10.7 Hz, |
| | (trifluoromethyl)tetra- | | | | 1H), 4.31 (ddt, J = 13.8, 10.4, |
| | hydrofuran-2- | | | | 5.5 Hz, 2H), 4.22 (ddt, J = 9.1, |
| | carboxamido)-1-methyl- | | | | 5.1, 2.5 Hz, 1H), 3.62 (s, 3H), |
| | 1H-pyrazole-3- | | | | 3.54 (td, J = 4.5, 1.9 Hz, 4H), |
| | carboxamide | | | | 2.89 (t, J = 7.4 Hz, 1H), 2.67 |
| | | | | | (t, J = 5.2 Hz, 2H), 2.42 (t, J = |
| | | | | | 4.7 Hz, 4H), 1.64 (s, 3H), 0.74- |
| | | | | | 0.69 (m, 3H) ppm. |
| 593 | 4-((2R,3S,4S,5R)-3-(3,4- | 575.528 | 576.3 | 3.12 | ¹H NMR (500 MHz, DMSO- |
| | difluoro-2-(2- | | | | d₆) δ 9.94 (s, 1H), 9.65-9.60 |
| | morpholinoethoxy)phenyl)- | | | | (m, 1H), 8.35 (s, 1H), 7.64 (s, |
| | 4,5-dimethyl-5- | | | | 1H), 7.18-7.13 (m, 2H), 5.20 |
| | (trifluoromethyl)tetra- | | | | (d, J = 10.6 Hz, 1H), 4.31 |
| | hydrofuran-2- | | | | (ddd, J = 10.2, 6.2, 3.9 Hz, |
| | carboxamido)-3-methyl- | | | | 2H), 4.24-4.19 (m, 1H), 3.53 |
| | 1H-pyrazole-1- | | | | (td, J = 4.3, 2.2 Hz, 4H), 2.88 |
| | carboxamide | | | | (q, J = 7.4 Hz, 1H), 2.66 (t, J = |
| | | | | | 5.2 Hz, 2H), 2.41 (d, J = 5.3 |
| | | | | | Hz, 4H), 2.19 (s, 3H), 1.61 (d, |
| | | | | | J = 5.3 Hz, 3H), 0.71 (s, 3H) ppm. |
| 594 | (2R,3S,4S,5R)-3-(3,4- | 588.567 | 589.2 | 3.11 | ¹H NMR (500 MHz, DMSO- |
| | difluoro-2-(2- | | | | d₆) δ 9.55 (s, 1H), 8.00 (s, 1H), |
| | morpholinoethoxy)phenyl)- | | | | 7.19-7.09 (m, 2H), 5.43 (tt, |
| | 4,5-dimethyl-N-(3- | | | | J = 7.6, 6.3 Hz, 1H), 5.16 (d, J = |
| | methyl-1-(oxetan-3-yl)- | | | | 10.7 Hz, 1H), 4.88-4.75 (m, |
| | 1H-pyrazol-4-yl)-5- | | | | 4H), 4.35-4.18 (m, 3H), 3.54 |
| | (trifluoromethyl)tetra- | | | | (td, J = 4.5, 2.2 Hz, 4H), 2.88 |
| | hydrofuran-2-carboxamide | | | | (p, J = 7.4 Hz, 1H), 2.66 (t, J = |
| | | | | | 5.3 Hz, 2H), 2.42 (t, J = 4.7 |
| | | | | | Hz, 4H), 2.13 (s, 3H), 1.60 (s, |
| | | | | | 3H), 0.74-0.68 (m, 3H) ppm. |

The following compounds were made using methods similar to those described in Example 17, except that rac-2-[1-[tert-butyl(dimethyl)silyl]oxy-2-methoxy-ethyl]pyridin-4-amine was used in amide coupling step 4. The diastereomers generated in step 4 were separated by chiral SFC and General method J was used as the final step on the second eluting isomer. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 595 | rel-(2R*,3S*,4S*,5R*)- | 603.578 | 604.3 | 3.10 | ¹H NMR (500 MHz, DMSO- |
| | 3-(3,4-difluoro-2-(2- | | | | d₆) δ 10.54 (s, 1H), 8.34 (d, J = |
| | morpholinoethoxy)phenyl)- | | | | 5.5 Hz, 1H), 7.75 (d, J = 2.0 |
| | N-(2-(1-hydroxy-2- | | | | Hz, 1H), 7.53 (dd, J = 5.5, 2.2 |
| | methoxyethyl)pyridin-4- | | | | Hz, 1H), 7.19-7.09 (m, 2H), |
| | yl)-4,5-dimethyl-5- | | | | 5.52 (d, J = 4.1 Hz, 1H), 5.11 |
| | (trifluoromethyl)tetra- | | | | (d, J = 10.6 Hz, 1H), 4.70- |
| | hydrofuran-2-carboxamide | | | | 4.65 (m, 1H), 4.33 (td, J = |
| | (precursor was second | | | | 10.3, 9.8, 6.2 Hz, 2H), 4.22 |
| | eluting isomer by SFC | | | | (dt, J = 12.0, 5.6 Hz, 1H), 3.63- |
| | using a Whelk-01 | | | | 3.39 (m, 6H), 3.25 (s, 3H), |
| | column) | | | | 2.88 (p, J = 7.4 Hz, 1H), 2.65 |
| | | | | | (t, J = 5.2 Hz, 2H), 2.40 (d, J = |
| | | | | | 4.9 Hz, 4H), 1.63 (s, 3H), 0.71 |
| | | | | | (d, J = 7.2 Hz, 3H) ppm. |

The following compounds were made using methods similar to those described in Example 17, except that 2-[(2S)-2-[tert-butyl(dimethyl)silyl]oxy-3-methoxy-propyl]pyridin-4-amine was used in amide coupling step 4 and General method J was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 596 | (2R,3S,4S,5R)-3-(3,4- | 617.605 | 618.2 | 3.11 | ¹H NMR (500 MHz, DMSO- |
| | difluoro-2-(2- | | | | d₆) δ 10.46 (s, 1H), 7.53- |
| | morpholinoethoxy)phenyl)- | | | | 7.43 (m, 2H), 7.19-7.09 (m, |
| | N-(2-((S)-2-hydroxy- | | | | 2H), 5.12 (d, J = 10.5 Hz, |
| | 3-methoxypropyl)pyridin- | | | | 1H), 4.80 (d, J = 5.2 Hz, 1H), |
| | 4-yl)-4,5-dimethyl-5- | | | | 4.38-4.28 (m, 2H), 4.26- |
| | (trifluoromethyl)tetra- | | | | 4.18 (m, 1H), 4.02-3.92 (m, |
| | hydrofuran-2-carboxamide | | | | 1H), 3.51 (h, J = 6.7 Hz, 8H), |
| | | | | | 3.26 (d, J = 5.7 Hz, 3H), |
| | | | | | 2.93-2.77 (m, 2H), 2.69-2.60 |
| | | | | | (m, 3H), 2.40 (d, J = 5.6 Hz, |
| | | | | | 3H), 1.63 (s, 3H), 0.71 (d, J = |
| | | | | | 7.4 Hz, 3H) ppm. |

The following compound was made using a method similar to that described in Example 17, except that (3aS,6aR)-5-(2-chloroethyl)-1,3,3a,4,6,6a-hexahydrofuro[3,4-c]pyrrole was as used as the alkylating agent in step 2, 5-amino-1-methyl-pyrazole-3-carboxamide was used in the amide coupling step 4. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 597 | 5-((2R,3S,4S,5R)-3-(3,4- | 601.565 | 602.1 | 3.00 | ¹H NMR (500 MHz, DMSO- |
| | difluoro-2-(2- | | | | d₆) δ 10.29 (s, 1H), 7.40 (s, |
| | ((3aR,6aS)-tetrahydro- | | | | 1H), 7.23-7.12 (m, 3H), |
| | 1H-furo[3,4-c]pyrrol- | | | | 6.52 (s, 1H), 5.18 (d, J = 10.6 |
| | 5(3H)-yl)ethoxy)phenyl)- | | | | Hz, 1H), 4.30-4.24 (m, 2H), |
| | 4,5-dimethyl-5- | | | | 4.19-4.14 (m, 1H), 3.64- |
| | (trifluoromethyl)tetra- | | | | 3.57 (m, 2H), 3.61 (s, 3H), |
| | hydrofuran-2- | | | | 3.38 (dd, J = 8.6, 2.7 Hz, |
| | carboxamido)-1-methyl- | | | | 1H), 3.33 (dd, J = 8.6, 2.7 |
| | 1H-pyrazole-3- | | | | Hz, 1H), 2.83 (p, J = 7.5 Hz, |
| | carboxamide | | | | 1H), 2.73-2.65 (m, 6H), |
| | | | | | 2.25-2.19 (m, 2H), 1.62 (s, |
| | | | | | 3H), 0.71 (d, J = 6.4 Hz, 3H) ppm. |

### Example 18

(2R,3S,4S,5R)-3-(2-(2-(2-oxa-6-azaspiro[3.3]heptan-6-yl)ethoxy)-3,4-difluorophenyl)-N-(1-(difluoromethyl)-3-methyl-1H-pyrazol-4-yl)-4,5-dimethyl-5-(trifluoromethyl)tetrahydrofuran-2-carboxamide (598)

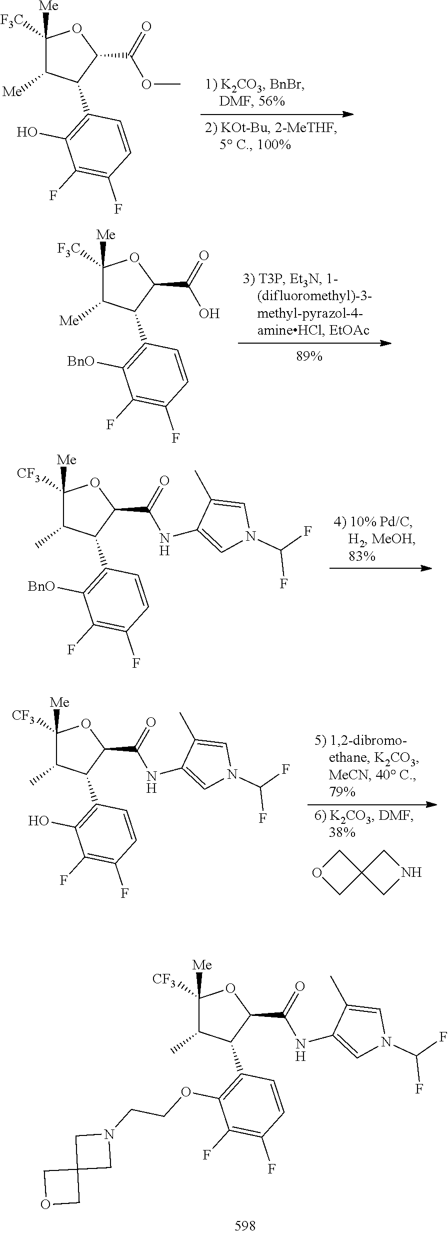

### Step 1:

To a solution of methyl (2S,3S,4S,5R)-3-(3,4-difluoro-2-hydroxy-phenyl)-4,5-dimethyl-5-(trifluoromethyl)tetrahydrofuran-2-carboxylate (13.5 g, 38.1 mmol) in DMF (50 mL) was added K₂CO₃ (6.85 g, 49.564 mmol) and benzyl bromide (9.778 g, 6.8 mL, 57.172 mmol). The reaction mixture was stirred at ambient temperature overnight. A further portion of K₂CO₃ (2.6 g, 18.81 mmol) and benzyl bromide (3.34 g, 2.3 mL, 19.33 mmol) were added and reaction mixture was stirred at ambient temperature overnight. The reaction mixture was diluted with water (100 mL) and extracted with diethyl ether (3×100 mL). The combined organic extracts were washed with brine (50 mL), dried (Na₂SO₄), filtered and concentrated in vacuo. Purification by flash chromatography (SiO₂, 0 to 50% EtOAc in heptane) gave a 2:3 mixture of methyl and benzyl (2S,3S,4S,5R)-3-(2-benzyloxy-3,4-difluoro-phenyl)-4,5-dimethyl-5-(trifluoromethyl)tetrahydrofuran-2-carboxylate (12.2 g) as a yellow oil.

### Step 2:

To a solution of a 2:3 ratio of methyl and benzyl (2S,3S,4S,5R)-3-(2-benzyloxy-3,4-difluoro-phenyl)-4,5-dimethyl-5-(trifluoromethyl)tetrahydrofuran-2-carboxylate (1.5 g, 1.3 mmol) in 2-MeTHF (5 mL) was added KO-t-Bu (438 mg, 3.9 mmol) at 0° C. under argon. The reaction mixture was stirred at 0° C. for 15 min then ambient temperature for 30 min. The reaction mixture was diluted with diethyl ether (20 mL) and acidified with 2 M HCl. The aqueous phase was extracted with diethyl ether (20 mL) and the combined organic extracts were washed with brine (20 mL), dried (Na₂SO₄), filtered and concentrated in vacuo. Purification by reverse phase chromatography (120 g C18, 0 to 100% MeCN) gave (2R,3S,4S,5R)-3-(2-benzyloxy-3,4-difluoro-phenyl)-4,5-dimethyl-5-(trifluoromethyl)tetrahydrofuran-2-carboxylic acid (760 mg) as a white solid. ¹H NMR (400 MHz, chloroform-d) δ 7.37-7.33 (m, 5H), 6.99-6.87 (m, 2H), 5.23 (d, J=11.0 Hz, 1H), 5.07 (d, J=11.0 Hz, 1H), 4.83 (d, J=11.0 Hz, 1H), 3.91 (dd, J=11.0, 7.8 Hz, 1H), 2.44 (dd, J=15.3, 7.6 Hz, 1H), 1.37 (s, 3H), 0.66 (dd, J=7.3, 2.3 Hz, 3H) ppm; alcohol OH not observed. ESI-MS m/z calc. 430.1204, found 429.04 (M−1)⁻.

### Step 3:

To a solution of (2R,3S,4S,5R)-3-(2-benzyloxy-3,4-difluoro-phenyl)-4,5-dimethyl-5-(trifluoromethyl)tetrahydrofuran-2-carboxylic acid (4 g, 2.93 mmol) 1-(difluoromethyl)-3-methyl-pyrazol-4-amine (1.4 g, 9.51 mmol), and Et₃N (1.9602 g, 2.7 mL, 19.371 mmol) in EtOAc (30 mL) was added T3P solution (20 mL of 50% w/v in EtOAc, 16.79 mmol). The reaction mixture was stirred at ambient temperature for 4 h. The reaction mixture was partitioned between EtOAc (80 mL) and water (15 mL). The organic layer was dried (MgSO₄), filtered, and concentrated in vacuo to give a yellow oil. Purification by flash chromatography (SiO₂, 25% EtOAc in heptanes) gave (2R,3S,4S,5R)-3-(2-benzyloxy-3,4-difluoro-phenyl)-N-[1-(difluoromethyl)-3-methyl-pyrazol-4-yl]-4,5-dimethyl-5-(trifluoromethyl)tetrahydrofuran-2-carboxamide (1.7 g, 89%) as a beige solid. ¹H NMR (400 MHz, Chloroform-d) δ 8.31 (s, 1H), 7.96 (s, 1H), 7.31-7.17 (m, 5H), 7.11-6.88 (m, 3H), 5.25 (d, J=11.4 Hz, 1H), 5.05 (d, J=11.4 Hz, 1H), 4.92 (d, J=11.4 Hz, 1H), 3.74 (dd, J=11.4, 7.3 Hz, 1H), 2.48 (q, J=7.3 Hz, 1H), 2.25 (s, 3H), 1.39 (s, 3H), 0.71-0.69 (m, 3H) ppm. ESI-MS m/z calc. 559.1706, found 560.2 (M+1); Retention time: 3.18 minutes. ¹⁹F-NMR (376 MHz, Chloroform-d) δ−74.8 (s, 3F), −93.3 (ddd, J=449.0, 227.1, 60.6 Hz, 2F), −136.9 (qd, J=9.8, 5.7 Hz, 1F), −153.0 (dd, J=19.7, 7.1 Hz, 1F) ppm.

### Step 4:

To a solution of (2R,3S,4S,5R)-3-(2-benzyloxy-3,4-difluoro-phenyl)-N-[1-(difluoromethyl)-3-methyl-pyrazol-4-yl]-4,5-dimethyl-5-(trifluoromethyl)tetrahydrofuran-2-carboxamide (1.5 g, 2.31 mmol) in EtOH (20 mL) was added palladium on carbon 10% (50 mg, 0.4698 mmol). The reaction mixture was stirred under a hydrogen atmosphere at ambient temperature for 2 h. The reaction mixture was filtered and concentrated in vacuo to give (2R,3S,4S,5R)-3-(3,4-difluoro-2-hydroxy-phenyl)-N-[1-(difluoromethyl)-3-methyl-pyrazol-4-yl]-4,5-dimethyl-5-(trifluoromethyl)tetrahydrofuran-2-carboxamide (1.01 g, 82%) as a white solid. ¹H NMR (301 MHz, Chloroform-d) δ 8.32 (s, 1H), 8.15 (s, 1H), 7.22-6.73 (m, 3H), 5.76 (br s, 1H), 5.12 (d, J=11.0 Hz, 1H), 4.09 (dd, J=10.8, 7.7 Hz, 1H), 2.90-2.80 (m, 1H), 2.28 (s, 3H), 1.67 (s, 3H), 0.83-0.80 (m, 3H) ppm. ESI-MS m/z calc. 469.1236, found 468.1 (M−1)⁻; Retention time: 1.32 minutes, ¹⁹F-NMR (283 MHz, Chloroform-d) δ−74.6 (s, 3F), −93.4 (dd, J=60.5, 54.4 Hz, 2F), −137.9 (ddd, J=21.2, 9.6, 5.5 Hz, 1F), −163.0-−163.1 (m, 1F) ppm.

### Step 5:

A suspension of (2R,3S,4S,5R)-3-(3,4-difluoro-2-hydroxy-phenyl)-N-[1-(difluoromethyl)-3-methyl-pyrazol-4-yl]-4,5-dimethyl-5-(trifluoromethyl)tetrahydrofuran-2-carboxamide (100 mg, 0.1832 mmol), 1,2-dibromoethane (130.8 mg, 60 μL, 0.696 mmol), and K₂CO₃ (80 mg, 0.5788 mmol) in MeCN (4 mL) was stirred at 40° C. for 4 h. The reaction mixture was partitioned between EtOAc (30 mL) and water (10 mL). The organic layer was dried (MgSO₄), filtered, and concentrated in vacuo. Purification by flash column chromatography (SiO₂, 0 to 65% EtOAc in heptane) gave (2R,3S,4S,5R)-3-[2-(2-bromoethoxy)-3,4-difluoro-phenyl]-N-[1-(difluoromethyl)-3-methyl-pyrazol-4-yl]-4,5-dimethyl-5-(trifluoromethyl)tetrahydrofuran-2-carboxamide (98 mg, 78%) as a colourless oil. ESI-MS m/z calc. 575.0655, found 576.0 (M+1)⁺; Retention time: 2.93 minutes.

### Step 6:

A suspension of (2R,3S,4S,5R)-3-[2-(2-bromoethoxy)-3,4-difluoro-phenyl]-N-[1-(difluoromethyl)-3-methyl-pyrazol-4-yl]-4,5-dimethyl-5-(trifluoromethyl)tetrahydrofuran-2-carboxamide (60 mg, 0.0885 mmol), 2-oxa-6-azaspiro[3.3]heptane (10 mg, 0.1009 mmol), and K₂CO₃ (25 mg, 0.1809 mmol) in DMF (1 mL) was stirred at 50° C. for 4 hours. The reaction mixture was partitioned between EtOAc (15 mL) and water (5 mL). The organic layer was dried (MgSO₄), filtered and concentrated in vacuo. Purification by reverse phase chromatography (30 g C18, eluting with 10 to 100% MeCN in water) gave (2R,3S,4S,5R)-3-(2-(2-(2-oxa-6-azaspiro[3.3]heptan-6-yl)ethoxy)-3,4-difluorophenyl)-N-(1-(difluoromethyl)-3-methyl-1H-pyrazol-4-yl)-4,5-dimethyl-5-(trifluoromethyl)tetrahydrofuran-2-carboxamide (598.21 mg, 38%) as a white solid. ¹H NMR (400 MHz, Chloroform-d) δ 8.31 (s, 1H), 8.12 (s, 1H), 7.17-6.87 (m, 3H), 5.04 (d, J=11.0 Hz, 1H), 4.66 (t, J=7.3 Hz, 4H), 4.19-4.14 (m, 1H), 4.10-4.00 (m, 2H), 3.35 (dd, J=12.6, 7.6 Hz, 4H), 2.82 (t, J=7.6 Hz, 1H), 2.73-2.61 (m, 2H), 2.28 (s, 3H), 1.65 (s, 3H), 0.78-0.76 (m, 3H) pp. ESI-MS m/z calc. 594.2077, found 595.2 (M+1i; Retention time: 2.49 minutes, ¹⁹F-NMR (376 MHz, Chloroform-d) δ−74.7 (s, 3F), −93.4 (dd, f=98.9, 60.8 Hz, 2F), −137.0-−137.1 (i, 1F), −153.5 (dd, 2=19.2, 6.0 Hz, 9F) ppm.

The following compound was made using a method similar to that described in Example 18, except that tert-butyl 4-(bromomethyl)pyrazole-1-carboxylate was used in place of 1,2-dibromoethane in step 5 with heating at 60° C. and step 6 was replaced by General Method I as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 599 | (2R,3S,4S,5R)-3-(2- | 549.441 | 550.1 | 3.18 | ¹H NMR (500 MHz, |
| | ((1H-pyrazol-4- | | | | Chloroform-d) δ 8.49-8.22 |
| | yl)methoxy)-3,4- | | | | (m, 1H), 8.14-8.02 (m, |
| | difluorophenyl)-N-(1- | | | | 2H), 7.91 (d, J = 9.4 Hz, |
| | (difluoromethyl)-3- | | | | 1H), 7.18-7.07 (m, 1H), |
| | methyl-1H-pyrazol-4- | | | | 7.06-6.94 (m, 1H), 5.35- |
| | yl)-4,5-dimethyl-5- | | | | 5.05 (m, 2H), 5.02 (dd, J = |
| | (trifluoromethyl)tetrahy | | | | 9.9, 5.7 Hz, 2H), 4.16- |
| | drofuran-2-carboxamide | | | | 3.96 (m, 1H), 2.77-2.58 |
| | | | | | (m, 2H), 2.31 (d, J = 5.6 |
| | | | | | Hz, 3H), 1.60 (d, J = 7.6 |
| | | | | | Hz, 3H), 0.80 (dt, J = 7.5, |
| | | | | | 2.3 Hz, 3H) ppm. |

The following compound was made using a method similar to that described in Example 18, except that (1r,3r)-3-((tert-butyldimethylsilyl)oxy)cyclobutyl methane sulfonate in DMF at 110° C. was used in place of 1,2-dibromoethane in step 5 and step 6 was replaced by General Method I as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 600 | (2R,3S,4S,5R)-3-(3,4- | 539.443 | 540.6 | 3.28 | ¹H NMR (500 MHz, |
| | difluoro-2-((1s,3R)-3- | | | | Chloroform-d) δ 8.29 (s, 1H), |
| | hydroxycyclobutoxy)phenyl)- | | | | 8.10 (s, 1H), 7.17-6.99 (m, |
| | N-(1-(difluoromethyl)- | | | | 2H), 6.89 (q, J = 8.9 Hz, 1H), |
| | 3-methyl-1H-pyrazol-4- | | | | 5.03 (d, J = 10.9 Hz, 1H), |
| | yl)-4,5-dimethyl-5- | | | | 4.39 (pd, J = 6.8, 2.5 Hz, |
| | (trifluoromethyl)tetra- | | | | 1H), 4.14 (dd, J = 10.9, 7.8 |
| | hydrofuran-2-carboxamide | | | | Hz, 1H), 3.97 (p, J = 6.9 Hz, |
| | | | | | 1H), 2.98-2.88 (m, 1H), |
| | | | | | 2.80 (ddd, J = 11.3, 9.9, 5.7 |
| | | | | | Hz, 2H), 2.29 (s, 3H), 2.19 |
| | | | | | (dt, J = 12.9, 7.0 Hz, 1H), |
| | | | | | 2.09 (dt, J = 12.3, 7.1 Hz, |
| | | | | | 1H), 1.67 (s, 3H), 0.80 (dq, |
| | | | | | J = 7.3, 2.3 Hz, 3H) ppm. |

The following compound was made using a method similar to that described in Example 18, except that step 5 alkylation was carried out under standard Mitsunobu conditions (2-methylenepropane-1,3-diol, PPh₃, DIAD, DCM) and step 6 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 601 | (2R,3S,4S,5R)-3-(3,4- | 539.443 | 540.2 | 2.55 | ¹H NMR (500 MHz, |
| | difluoro-2-((2- | | | | Chloroform-d) δ 8.28 (s, 1H), |
| | (hydroxymethyl)allyl)oxy)phenyl)- | | | | 8.10 (s, 1H), 7.11-7.07 (m, |
| | N-(1-(difluoromethyl)-3- | | | | 1H), 7.02 (t, J= 60.8 Hz, 1H), |
| | methyl-1H-pyrazol-4- | | | | 6.93 (td, J = 9.2, 7.8 Hz, 1H), |
| | yl)-4,5-dimethyl-5- | | | | 5.25 (d, J = 1.4 Hz, 1H), 5.22 |
| | (trifluoromethyl)tetra- | | | | (d, J = 0.9 Hz, 1H), 5.01 (d, |
| | hydrofuran-2-carboxamide | | | | J = 10.5 Hz, 1H), 4.74 (d, J = |
| | | | | | 11.4 Hz, 1H), 4.61 (d, J = |
| | | | | | 11.9 Hz, 1H), 4.26-4.16 (m, |
| | | | | | 3H), 2.72 (q, J = 7.6 Hz, 1H), |
| | | | | | 2.27 (s, 3H), 1.87 (s, 1H), |
| | | | | | 1.63 (s, 3H), 0.81-0.78 (m, |
| | | | | | 3H) ppm |

The following compound was made using a method similar to that described in Example 18, except that methyl 2-bromoacetate was used in place of 1,2-dibromoethane in step 5 and step 6 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 602 | methyl 2-(6- | 541.416 | 542 | 3.52 | ¹H NMR (500 MHz, DMSO-d6) |
| | ((2R,3S,4S,5R)-2-((1- | | | | 8 9.94 (s, 1H), 8.31 (s, 1H), 7.63 |
| | (difluoromethyl)-3- | | | | (t, J = 59.3 Hz, 1H), 7.17 (t, J = |
| | methyl-1H-pyrazol-4- | | | | 6.4 Hz, 2H), 5.19 (d, J = 10.7 |
| | yl)carbamoyl)-4,5- | | | | Hz, 1H), 5.03-4.84 (m, 2H), |
| | dimethyl-5- | | | | 4.39 (dd, J = 10.7, 7.1 Hz, 1H), |
| | (trifluoromethyl)tetra- | | | | 3.72 (s, 3H), 2.94-2.85 (m, |
| | hydrofuran-3-yl)-2,3- | | | | 1H), 2.18 (s, 3H), 1.61 (s, 3H), |
| | difluorophenoxy)acetate | | | | 0.79-0.65 (m, 3H) ppm. |

The following compound was made from 602 using General Method O. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 603 | 2-(6-((2R,3S,4S,5R)-2- | 527.389 | 528.3 | 2.26 | ¹H NMR (500 MHz, DMSO- |
| | ((1-(difluoromethyl)-3- | | | | d₆) 8 13.11 (s, 1H), 9.92 (s, |
| | methyl-1H-pyrazol-4- | | | | 1H), 8.29 (s, 1H), 7.61 (s, |
| | yl)carbamoyl)-4,5- | | | | 1H), 7.14 (t, J = 5.3 Hz, 2H), |
| | dimethyl-5- | | | | 5.17 (d, J = 10.7 Hz, 1H), |
| | (trifluoromethyl)tetra- | | | | 4.91-4.70 (m, 2H), 4.38 (dd, |
| | hydrofuran-3-yl)-2,3- | | | | J = 10.7, 7.2 Hz, 1H), 2.91 |
| | difluorophenoxy)acetic | | | | (q, J = 7.3 Hz, 1H), 2.17 (s, |
| | acid | | | | 3H), 1.58 (s, 3H), 0.71 (d, J = |
| | | | | | 7.3 Hz, 3H) ppm. |

The following compound was made from 603 using HATU coupling conditions similar to Example 8, step 1. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 604 | (2R,3S,4S,5R)-3-(3,4- | 556.431 | 557.3 | 3.09 | ¹H NMR (500 MHz, DMSO- |
| | difluoro-2-(2- | | | | d₆) δ 9.98 (s, 1H), 9.87 (s, |
| | (hydroxy(methyl)amino)- | | | | 1H), 8.28 (s, 1H), 7.17-7.05 |
| | 2-oxoethoxy)phenyl)- | | | | (m, 2H), 5.16 (d, J = 10.8 Hz, |
| | N-(1-(difluoromethyl)- | | | | 1H), 5.11-4.84 (m, 3H), |
| | 3-methyl-1H-pyrazol-4- | | | | 4.43 (dd, J = 10.7, 7.2 Hz, |
| | yl)-4,5-dimethyl-5- | | | | 1H), 3.10 (s, 3H), 2.92 (t, J = |
| | (trifluoromethyl)tetra- | | | | 7.3 Hz, 1H), 2.17 (s, 3H), |
| | hydrofuran-2-carboxamide | | | | 1.59 (s, 3H), 0.71 (d, J = 7.3 |
| | | | | | Hz, 3H) ppm. |

The following compounds were made from 603 via amide coupling reactions using different amines with ethylcarbon chloride and 4-methyl morpholine in diethyl ether at ambient temperature. In the

| Cmpd | | LC/MS | Found | MS | |
|---|---|---|---|---|---|
| No. | Compound Name | (m/z calc.) | M + 1 | r.t. | NMR (shifts in ppm) |
| 605 | (2R,3S,4S,5R)-3-(2-(2- | 526.405 | 527.3 | 2.94 | ¹H NMR (500 MHz, DMSO- |
| | amino-2-oxoethoxy)- | | | | d₆) δ 9.88 (s, 1H), 8.29 (s, |
| | 3,4-difluorophenyl)-N- | | | | 1H), 7.76-7.35 (m, 3H), |
| | (1-(difluoromethyl)-3- | | | | 7.17 (q, J = 8.5, 7.9 Hz, 2H), |
| | methyl-1H-pyrazol-4- | | | | 5.17 (d, J = 10.8 Hz, 1H), |
| | yl)-4,5-dimethyl-5- | | | | 4.61-4.46 (m, 2H), 4.37 (dd, |
| | (trifluoromethyl)tetra- | | | | J = 10.8, 7.3 Hz, 1H), 2.92 |
| | hydrofuran-2-carboxamide | | | | (p, J = 7.6 Hz, 1H), 2.17 (s, |
| | | | | | 3H), 1.59 (s, 3H), 0.76-0.65 |
| | | | | | (m, 3H) ppm. |
| 606 | (2R,3S,4S,5R)-3-(3,4- | 542.404 | 543.4 | 3.01 | ¹H NMR (500 MHz, DMSO- |
| | difluoro-2-(2- | | | | d₆) δ 10.88 (s, 1H), 9.89 (s, |
| | (hydroxy(methyl)amino)- | | | | 1H), 9.03 (s, 1H), 8.30 (s, |
| | 2-oxoethoxy)phenyl)- | | | | 1H), 7.63 (t, J = 59.2 Hz, |
| | N-(1-(difluoromethyl)- | | | | 1H), 7.18 (dd, J = 10.5, 6.5 |
| | 3-methyl-1H-pyrazol-4- | | | | Hz, 2H), 5.17 (d, J = 10.8 |
| | yl)-4,5-dimethyl-5- | | | | Hz, 1H), 4.54 (s, 2H), 4.38 |
| | (trifluoromethyl)tetra- | | | | (dd, J = 10.8, 7.1 Hz, 1H), |
| | hydrofuran-2-carboxamide | | | | 2.95 (p, J = 7.3 Hz, 1H), 2.17 |
| | | | | | (s, 3H), 1.61 (s, 3H), 0.78- |
| | | | | | 0.66 (m, 3H) ppm. |
| 607 | (2R,3S,4S,5R)-3-(3,4- | 540.431 | 541.4 | 3.05 | ¹H NMR (500 MHz, DMSO- |
| | difluoro-2-(2- | | | | d₆) δ 9.89 (s, 1H), 8.30 (s, |
| | (methylamino)-2- | | | | 1H), 8.15 (s, 1H), 7.63 (t, J = |
| | oxoethoxy)phenyl)-N- | | | | 59.2 Hz, 1H), 7.23-7.12 (m, |
| | (1-(difluoromethyl)-3- | | | | 2H), 5.18 (d, J = 10.8 Hz, |
| | methyl-1H-pyrazol-4- | | | | 1H), 4.60-4.47 (m, 2H), |
| | yl)-4,5-dimethyl-5- | | | | 4.36 (dd, J = 10.9, 7.2 Hz, |
| | (trifluoromethyl)tetra- | | | | 1H), 2.88 (q, J = 7.3 Hz, 1H), |
| | hydrofuran-2-carboxamide | | | | 2.67 (d, J = 4.6 Hz, 3H), 2.18 |
| | | | | | (s, 3H), 1.60 (s, 3H), 0.73 (d, |
| | | | | | J = 7.2 Hz, 3H) ppm. |

The following compounds were made using a method similar to that described in Example 18, except that rac-2-[1-[tert-butyl(dimethyl)silyl]oxy-2-methoxy-ethyl]pyridin-4-amine was used as the amine in step 3 and different amines were used in step 6. Diastereomers were separated by chiral SFC after step 6 and TBS deprotection using General Method M was carried out on separated isomers as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 608 | rel-(2R*,3S*,4S*,5R*)- | 591.543 | 591.9 | 3.19 | ¹H NMR (500 MHz, DMSO- |
| | 3-(3,4-difluoro-2-(2-(3- | | | | d₆) δ 10.54 (s, 1H), 8.34 (d, J = |
| | fluoroazetidin-1- | | | | 5.5 Hz, 1H), 7.73 (d, J = 2.2 |
| | yl)ethoxy)phenyl)-N-(2- | | | | Hz, 1H), 7.53 (dd, J = 5.5, 2.2 |
| | (1-hydroxy-2- | | | | Hz, 1H), 7.19-7.12 (m, 2H), |
| | methoxyethyl)pyridin-4- | | | | 5.52 (d, J = 4.7 Hz, 1H), 5.17- |
| | yl)-4,5-dimethyl-5- | | | | 5.01 (m, 1H), 5.09 (d, J = 10.7 |
| | (trifluoromethyl)tetra- | | | | Hz, 1H), 4.41 (dd, J = 10.7, 7.3 |
| | hydrofuran-2-carboxamide | | | | Hz, 1H), 4.17-4.12 (m, 1H), |
| | (first eluting isomer by | | | | 4.02-3.97 (m, 1H), 3.62- |
| | SFC suing Whelk-01 | | | | 3.55 (m, 3H), 3.42 (dd, J = |
| | column) | | | | 10.1, 7.0 Hz, 1H), 3.24 (s, 3H), |
| | | | | | 3.22-3.07 (m, 2H), 2.89- |
| | | | | | 2.84 (m, 1H), 2.83-2.74 (m, |
| | | | | | 2H), 1.64 (s, 3H), 0.70 (d, J = |
| | | | | | 6.2 Hz, 3H) ppm. |
| 609 | rel-(2R*,3S*,4S*,5R*)- | 591.543 | 591.9 | 3.19 | ¹H NMR (500 MHz, DMSO- |
| | 3-(3,4-difluoro-2-(2- | | | | d₆) δ 10.53 (s, 1H), 8.34 (d, J = |
| | (3-fluoroazetidin-1- | | | | 5.5 Hz, 1H), 7.74 (d, J = 2.1 |
| | yl)ethoxy)phenyl)-N- | | | | Hz, 1H), 7.53 (dd, J = 5.5, 2.1 |
| | (2-(1-hydroxy-2- | | | | Hz, 1H), 7.19-7.11 (m, 2H), |
| | methoxyethyl)pyridin- | | | | 5.52 (d, J = 4.7 Hz, 1H), 5.18- |
| | 4-yl)-4,5-dimethyl-5- | | | | 5.01 (m, 1H), 5.09 (d, J = 10.7 |
| | (trifluoromethyl)tetra- | | | | Hz, 1H), 4.69-4.65 (m, 1H), |
| | hydrofuran-2-carboxamide | | | | 4.41 (dd, J = 10.7, 7.3 Hz, 1H), |
| | (second eluting isomer | | | | 4.16-4.11 (m, 1H), 4.02- |
| | by SFC suing Whelk-01 | | | | 3.97 (m, 1H), 3.63-3.54 (m, |
| | column) | | | | 3H), 3.42 (dd, J = 10.1, 7.0 Hz, |
| | | | | | 1H), 3.24 (s, 3H), 3.22-3.07 |
| | | | | | (m, 2H), 2.89-2.84 (m, 1H), |
| | | | | | 2.83-2.74 (m, 2H), 1.64 (s, |
| | | | | | 3H), 0.70 (d, J = 6.3 Hz, 3H) |
| | | | | | ppm. |
| 610 | rel-(2R*,3S*,4S*,5R*)- | 615.589 | 615.9 | 3.12 | ¹H NMR (500 MHz, DMSO- |
| | 3-(2-(2-(6-oxa-3- | | | | d₆) δ 10.52 (s, 1H), 8.34 (d, J = |
| | azabicyclo[3.1.1]heptan- | | | | 5.5 Hz, 1H), 7.73 (d, J = 2.1 |
| | 3-yl)ethoxy)-3,4- | | | | Hz, 1H), 7.52 (dd, J = 5.5, 2.1 |
| | difluorophenyl)-N-(2- | | | | Hz, 1H), 7.17-7.11 (m, 2H), |
| | (1-hydroxy-2- | | | | 5.52 (d, J = 4.5 Hz, 1H), 5.10 |
| | methoxyethyl)pyridin-4- | | | | (d, J = 10.3 Hz, 1H), 4.69- |
| | yl)-4,5-dimethyl-5- | | | | 4.65 (m, 1H), 4.38-4.31 (m, |
| | (trifluoromethyl)tetra- | | | | 3H), 4.30-4.25 (m, 2H), 3.59 |
| | hydrofuran-2-carboxamide | | | | (dd, J = 10.1, 3.6 Hz, 1H), 3.43 |
| | (first eluting isomer by | | | | (dd, J = 10.1, 6.9 Hz, 1H), 3.24 |
| | SFC suing Whelk-01 | | | | (s, 3H), 3.07 (dd, J = 11.2, 2.3 |
| | column) | | | | Hz, 1H), 3.01 (dd, J = 11.2, 2.3 |
| | | | | | Hz, 1H), 2.98-2.87 (m, 2H), |
| | | | | | 2.82 (dq, J = 7.5 Hz, 7.5 Hz, |
| | | | | | 1H), 2.74-2.69 (m, 1H), 2.66 |
| | | | | | (dd, J = 11.2, 1.3 Hz, 1H), 2.61 |
| | | | | | (dd, J = 11.2, 1.3 Hz, 1H), 2.03 |
| | | | | | (d, J = 7.8 Hz, 1H), 1.59 (s, |
| | | | | | 3H), 0.70 (d, J = 6.2 Hz, 3H) |
| | | | | | ppm. |
| 611 | rel-(2R*,3S*,4S*,5R*)- | 615.589 | 615.9 | 3.12 | ¹H NMR (500 MHz, DMSO- |
| | 3-(2-(2-(6-oxa-3- | | | | d₆) δ 10.52 (s, 1H), 8.34 (d, J = |
| | azabicyclo[3.1.1]heptan- | | | | 5.5 Hz, 1H), 7.74 (d, J = 2.1 |
| | 3-yl)ethoxy)-3,4- | | | | Hz, 1H), 7.51 (dd, J = 5.5, 2.1 |
| | difluorophenyl)-N-(2-(1- | | | | Hz, 1H), 7.16-7.12 (m, 2H), |
| | hydroxy-2- | | | | 5.52 (d, J = 4.5 Hz, 1H), 5.10 |
| | methoxyethyl)pyridin-4- | | | | (d, J = 10.3 Hz, 1H), 4.69- |
| | yl)-4,5-dimethyl-5- | | | | 4.66 (m, 1H), 4.38-4.25 (m, |
| | (trifluoromethyl)tetra- | | | | 5H), 3.59 (dd, J = 10.1, 3.6 Hz, |
| | hydrofuran-2-carboxamide | | | | 1H), 3.42 (dd, J = 10.1, 7.0 Hz, |
| | (second eluting isomer | | | | 1H), 3.24 (s, 3H), 3.07 (dd, J = |
| | by SFC suing Whelk-01 | | | | 11.2, 2.2 Hz, 1H), 3.01 (dd, J = |
| | column) | | | | 11.2, 2.2 Hz, 1H), 2.99-2.86 |
| | | | | | (m, 2H), 2.82 (dq, J = 7.5, 7.5 |
| | | | | | Hz, 1H), 2.73-2.69 (m, 1H), |
| | | | | | 2.67 (dd, J = 11.2, 1.3 Hz, 1H), |
| | | | | | 2.61 (dd, J = 11.2, 1.3 Hz, 1H), |
| | | | | | 2.02 (d, J = 7.8 Hz, 1H), 1.58 |
| | | | | | (s, 3H), 0.70 (d, J = 6.5 Hz, |
| | | | | | 3H) ppm. |

The following compounds were made using methods similar to those described in Example 18, except that methyl 5-aminopicolinate was used as the amine in step 3 and General Method L, using NH₃ or NHMe respectively, was used as the final step. In the Table below, “MS r.t.” stands for Mass Spec

| Cmpd | | LC/MS | Found | MS | |
|---|---|---|---|---|---|
| No. | Compound Name | (m/z calc.) | M + 1 | r.t. | NMR (shifts in ppm) |
| 612 | 5-((2R,3S,4S,5R)-3-(2- | 584.535 | 585 | 2.97 | ¹H NMR (500 MHz, DMSO- |
| | (2-(2-oxa-6- | | | | d₆) δ 10.67 (s, 1H), 8.81 (s, |
| | azaspiro[3.3]heptan-6- | | | | 1H), 8.23-8.15 (m, 1H), 7.97 |
| | yl)ethoxy)-3,4- | | | | (s, 2H), 7.48 (s, 1H), 7.16 (dq, |
| | difluorophenyl)-4,5- | | | | J = 16.6, 9.1 Hz, 2H), 5.11 (d, |
| | dimethyl-5- | | | | J = 10.7 Hz, 1H), 4.56 (q, J = |
| | (trifluoromethyl)tetra- | | | | 6.6 Hz, 4H), 4.48-4.34 (m, |
| | hydrofuran-2- | | | | 1H), 4.16-4.06 (m, 1H), 3.95 |
| | carboxamido)picolinami | | | | (dt, J = 10.4, 5.1 Hz, 1H), 2.85 |
| | de | | | | (p, J = 7.3 Hz, 1H), 2.68-2.61 |
| | | | | | (m, 2H), 1.63 (s, 3H), 0.70 (d, |
| | | | | | J = 7.3 Hz, 3H); water peak |
| | | | | | obscures 4H. |
| 613 | 5-((2R,3S,4S,5R)-3-(2- | 598.562 | 599 | 3.05 | ¹H NMR (500 MHz, DMSO- |
| | (2-(2-oxa-6- | | | | d₆) δ 10.63 (s, 1H), 8.88 (d, J = |
| | azaspiro[3.3]heptan-6- | | | | 2.4 Hz, 1H), 8.65 (d, J = 5.0 |
| | yl)ethoxy)-3,4- | | | | Hz, 1H), 8.21 (dd, J = 8.6, 2.5 |
| | difluorophenyl)-4,5- | | | | Hz, 1H), 7.99 (d, J = 8.5 Hz, |
| | dimethyl-5- | | | | 1H), 7.16 (dd, J = 9.8, 6.4 Hz, |
| | (trifluoromethyl)tetra- | | | | 2H), 5.15 (d, J = 10.8 Hz, 1H), |
| | hydrofuran-2- | | | | 4.55 (q, J = 6.6 Hz, 4H), 4.43 |
| | carboxamido)-N- | | | | (dd, J = 10.8, 7.2 Hz, 1H), |
| | methylpicolinamide | | | | 4.21-4.07 (m, 1H), 3.95 (dt, J = |
| | | | | | 10.4, 5.0 Hz, 1H), 2.85 (p, J = |
| | | | | | 7.5 Hz, 1H), 2.80 (d, J = 4.8 |
| | | | | | Hz, 3H), 2.64 (td, J = 6.1, 3.7 |
| | | | | | Hz, 2H), 1.64 (s, 3H), 0.71 (d, |
| | | | | | J = 6.8 Hz, 3H); water peak |
| | | | | | obscures 4H. |

The following compounds were made using methods similar to those described in Example 18, except that methyl 5-aminopicolinate was used as the amine in step 3 and different alkyl bromides (with heating at 80° C.) were used in step 3. Step 6 was omitted and General Method L was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 614 | 5-((2R,3S,4S,5R)-3-(3,4- | 531.472 | 532.7 | 3.14 | ¹H NMR (500 MHz, |
| | difluoro-2-((R)-3- | | | | Methanol-d₄) δ 8.85 (d, J = |
| | hydroxy-2- | | | | 2.4 Hz, 1H), 8.19 (dd, J = |
| | methylpropoxy)phenyl)- | | | | 8.6, 2.5 Hz, 1H), 8.05 (d, J = |
| | 4,5-dimethyl-5- | | | | 8.5 Hz, 1H), 7.15 (t, J = 7.3 |
| | (trifluoromethyl)tetra- | | | | Hz, 1H), 6.99 (q, J = 8.9 Hz, |
| | hydrofuran-2- | | | | 1H), 5.09 (d, J = 10.5 Hz, |
| | carboxamido)picolinamide | | | | 1H), 4.39 (dd, J = 10.5, 7.9 |
| | | | | | Hz, 1H), 4.22 (ddd, J = 9.1, |
| | | | | | 5.6, 1.7 Hz, 1H), 4.02-3.95 |
| | | | | | (m, 1H), 3.60 (qd, J = 10.8, |
| | | | | | 6.2 Hz, 2H), 2.80 (p, J = 7.5 |
| | | | | | Hz, 1H), 2.10 (h, J = 6.3 Hz, |
| | | | | | 1H), 1.68 (s, 3H), 1.09 (d, J = |
| | | | | | 6.9 Hz, 3H), 0.83 (dd, J = |
| | | | | | 7.7, 2.4 Hz, 3H) ppm. |
| 615 | 5-((2R,3S,4S,5R)-3-(3,4- | 503.419 | 504.3 | 2.9 | ¹H NMR (500 MHz, DMSO- |
| | difluoro-2-(2- | | | | d₆) δ 10.57 (s, 1H), 8.83 (d, |
| | hydroxyethoxy)phenyl)- | | | | J = 2.3 Hz, 1H), 8.20 (dd, J = |
| | 4,5-dimethyl-5- | | | | 8.6, 2.5 Hz, 1H), 8.01-7.97 |
| | (trifluoromethyl)tetra- | | | | (m, 2H), 7.53-7.49 (m, 1H), |
| | hydrofuran-2- | | | | 7.16 (dd, J = 8.5, 5.1 Hz, |
| | carboxamido)picolinamide | | | | 2H), 5.13 (d, J = 10.7 Hz, |
| | | | | | 1H), 4.96 (s, 1H), 4.41 (dd, |
| | | | | | J = 10.7, 7.3 Hz, 1H), 4.12 (qt, |
| | | | | | J = 9.9, 4.7 Hz, 2H), 3.71 (t, |
| | | | | | J = 4.7 Hz, 2H), 2.92 (p, J = |
| | | | | | 7.5 Hz, 1H), 1.61 (s, 3H), |
| | | | | | 0.74-0.69 (m, 3H) ppm. |
| 616 | 5-((2R,3S,4S,5R)-3-(3,4- | 531.472 | 532.3 | 3.12 | ¹H NMR (500 MHz, DMSO- |
| | difluoro-2-((S)-3- | | | | d₆) δ 10.66 (s, 1H), 8.81 (d, |
| | hydroxy-2- | | | | J = 2.4 Hz, 1H), 8.20 (dd, J = |
| | methylpropoxy)phenyl)- | | | | 8.6, 2.5 Hz, 1H), 8.01-7.97 |
| | 4,5-dimethyl-5- | | | | (m, 2H), 7.51 (s, 1H), 7.21- |
| | (trifluoromethyl)tetra- | | | | 7.11 (m, 2H), 5.13 (d, J = |
| | hydrofuran-2- | | | | 10.4 Hz, 1H), 4.61 (s, 1H), |
| | carboxamido)picolinamide | | | | 4.30 (dd, J = 10.4, 7.6 Hz, |
| | | | | | 1H), 4.06 (dd, J = 8.2, 3.5 |
| | | | | | Hz, 2H), 3.45 (dt, J = 9.1, 5.2 |
| | | | | | Hz, 2H), 2.77 (q, J = 7.3 Hz, |
| | | | | | 1H), 2.00 (h, J = 6.3 Hz, 1H), |
| | | | | | 1.61 (s, 3H), 1.01 (d, J = 6.8 |
| | | | | | Hz, 3H), 0.73 (d, J = 7.3 Hz, |
| | | | | | 3H) ppm. |

The following compounds were made using methods similar to those described in Example 18, except that different amines were used in the amide coupling step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 617 | (2R,3S,4S,5R)-3-(2-(2- | 581.534 | 582.21 | 2.22 | ¹H NMR (400 MHz, |
| | (2-oxa-6- | | | | Chloroform-d) δ 9.65 (s, 1H), |
| | azaspiro[3.3]heptan-6- | | | | 8.43 (s, 1H), 8.02 (s, 1H), |
| | yl)ethoxy)-3,4- | | | | 7.69 (d, J = 9.6 Hz, 1H), 7.10 |
| | difluorophenyl)-N- | | | | (t, J = 8.7 Hz, 2H), 6.99-6.91 |
| | ([1,2,3]triazolo[1,5- | | | | (m, 1H), 5.05 (d, J = 11.0 Hz, |
| | alpyridin-6-yl)-4,5- | | | | 1H), 4.65 (s, 4H), 4.28-4.05 |
| | dimethyl-5- | | | | (m, 3H), 3.36 (s, 4H), 2.85 |
| | (trifluoromethyl)tetra- | | | | (s, 1H), 2.73 (d, J = 7.3 Hz, |
| | hydrofuran-2- | | | | 2H), 1.74-1.66 (m, 3H), 0.80- |
| | carboxamide | | | | 0.77 (m, 3H) ppm. |
| 618 | (2R,3S,4S,5R)-3-(2-(2- | 571.536 | 572.3 | 2.9 | ¹H NMR (500 MHz, DMSO- |
| | (2-oxa-6- | | | | d₆) δ 10.59 (s, 1H), 9.9 (s, |
| | azaspiro[3.3]heptan-6- | | | | 1H), 8.34 (d, J = 5.5 Hz, 1H), |
| | yl)ethoxy)-3,4- | | | | 7.75-7.72 (m, 1H), 7.54- |
| | difluorophenyl)-N-(2- | | | | 7.49 (m, 1H), 7.26 (s, 1H), |
| | (hydroxymethyl)pyridin- | | | | 7.20 (s, 1H), 5.40 (t, J = 5.6 |
| | 4-yl)-4,5-dimethyl-5- | | | | Hz, 1H), 5.12 (d, J = 10.4 |
| | (trifluoromethyl)tetra- | | | | Hz, 1H), 4.77 (d, J = 9.0 Hz, |
| | hydrofuran-2- | | | | 2H), 4.62 (s, 2H), 4.51 (d, J = |
| | carboxamide | | | | 5.5 Hz, 2H), 4.40 (s, 3H), |
| | | | | | 4.25 (s, 3H), 3.61 (s, 2H), |
| | | | | | 2.79 (s, 1H), 1.64 (s, 3H), |
| | | | | | 0.73 (s, 3H) ppm. |
| 619 | (2R,3S,4S,5R)-3-(2-(2- | 545.499 | 545.9 | 3.11 | ¹H NMR (500 MHz, DMSO- |
| | (2-oxa-6- | | | | d₆) δ 10.05 (s, 1H), 9.04 (s, |
| | azaspiro[3.3]heptan-6- | | | | 1H), 7.17-7.09 (m, 2H), |
| | yl)ethoxy)-3,4- | | | | 5.18 (d, J = 10.8 Hz, 1H), |
| | difluorophenyl)-4,5- | | | | 4.57 (q, J = 6.6 Hz, 4H), 4.35 |
| | dimethyl-N-(3- | | | | (dd, J = 10.8, 7.2 Hz, 1H), |
| | methylisoxazol-4-yl)-5- | | | | 4.11-4.06 (m, 1H), 3.97- |
| | (trifluoromethyl)tetra- | | | | 3.92 (m, 1H), 3.34 (d, J = 7.2 |
| | hydrofuran-2- | | | | Hz, 2H), 3.29 (d, J = 7.2 Hz, |
| | carboxamide | | | | 2H), 2.90-2.84 (m, 1H), |
| | | | | | 2.68-2.60 (m, 2H), 2.23 (s, |
| | | | | | 3H), 1.60 (s, 3H), 0.69 (d, J = |
| | | | | | 6.3 Hz, 3H) ppm. |
| 620 | (2R,3S,4S,5R)-3-(2-(2- | 581.534 | 582.2 | 2.85 | ¹H NMR (500 MHz, DMSO- |
| | (2-oxa-6- | | | | d₆) δ 10.46 (s, 1H), 9.29- |
| | azaspiro[3.3]heptan-6- | | | | 9.22 (m, 2H), 7.78 (d, J = 9.8 |
| | yl)ethoxy)-3,4- | | | | Hz, 1H), 7.41 (dd, J = 9.8, |
| | difluorophenyl)-N- | | | | 1.9 Hz, 1H), 7.16 (dd, J = |
| | ([1,2,4]triazolo[4,3- | | | | 8.6, 4.3 Hz, 2H), 5.13 (d, J = |
| | a]pyridin-6-yl)-4,5- | | | | 10.8 Hz, 1H), 4.55 (q, J = 6.5 |
| | dimethyl-5- | | | | Hz, 4H), 4.40 (dd, J = 10.9, |
| | (trifluoromethyl)tetra- | | | | 7.1 Hz, 1H), 4.11 (dt, J = |
| | hydrofuran-2- | | | | 10.4, 5.0 Hz, 1H), 3.96 (dt, |
| | carboxamide | | | | J = 10.2, 4.8 Hz, 1H), 3.36- |
| | | | | | 3.27 (m, 4H), 2.86 (p, J = 7.4 |
| | | | | | Hz, 1H), 2.65 (ddd, J = 6.3, |
| | | | | | 4.1, 2.1 Hz, 2H), 1.64 (s, |
| | | | | | 3H), 0.74-0.68 (m, 3H) |
| | | | | | ppm. |

The following compound was made using a method similar to that described in Example 18, except that [1,2,3]triazolo[1,5-a]pyridin-6-amine was used as the amine in step 3 and 3-(bromomethyl)oxetane was used as alkylating agent in step 5. Step 6 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 621 | (2R,3S,4S,5R)-N- | 526.456 | 527.17 | 2.43 | ¹H NMR (400 MHz, |
| | ([1,2,3 ]triazolo[1,5- | | | | Chloroform-d) δ 9.61 (s, 1H), |
| | a]pyridin-6-yl)-3-(3,4- | | | | 8.34 (s, 1H), 8.02 (d, J = 1.4 |
| | difluoro-2-(oxetan-3- | | | | Hz, 1H), 7.69-7.67 (m, 1H), |
| | ylmethoxy)phenyl)-4,5- | | | | 7.12-7.07 (m, 2H), 6.96-6.94 |
| | dimethyl-5- | | | | (m, 1H), 5.05 (d, J = 11.0 Hz, |
| | (trifluoromethyl)tetra- | | | | 1H), 4.86 (dd, J = 8.0, 6.2 |
| | hydrofuran-2- | | | | Hz, 1H), 4.78 (dd, J = 8.0, |
| | carboxamide | | | | 6.2 Hz, 1H), 4.64 (t, J = 6.2 |
| | | | | | Hz, 1H), 4.53-4.49 (m, 2H), |
| | | | | | 4.30-4.23 (m, 2H), 3.35 (s, |
| | | | | | 1H), 2.76 (t, J = 7.6 Hz, 1H), |
| | | | | | 1.67 (d, J = 4.6 Hz, 3H), |
| | | | | | 0.82-0.80 (m, 3H) ppm. |

The following compound was made using a method similar to that described in Example 18, except that [1,2,3]triazolo[1,5-a]pyridin-6-amine was used as the amine in step 3 and step 5 alkylation was carried out under standard Mitsunobu conditions (2-methylenepropane-1,3-diol, PPh₃, DIAD, DCM). Step 6 was omitted. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 622 | (2R,3S,4S,5R)-N- | 526.456 | 527.18 | 2.33 | ¹H NMR (400 MHz, |
| | ([1,2,3 ]triazolo[1,5- | | | | Chloroform-d) δ 9.58 (s, 1H), |
| | a]pyridin-6-yl)-3-(3,4- | | | | 8.36 (s, 1H), 8.02 (d, J = 0.7 |
| | difluoro-2-((2- | | | | Hz, 1H), 7.68 (dd, J = 9.6, |
| | (hydroxymethyl)al- | | | | 0.9 Hz, 1H), 7.12-7.08 (m, |
| | lyl)oxy)phenyl)-4,5- | | | | 2H), 6.99-6.92 (m, 1H), 5.25 |
| | dimethyl-5- | | | | (d, J = 4.1 Hz, 2H), 5.03 (d, |
| | (trifluoromethyl)tetra- | | | | J = 10.5 Hz, 1H), 4.76 (d, J = |
| | hydrofuran-2-carboxamide | | | | 11.9 Hz, 1H), 4.64 (d, J = |
| | | | | | 11.4 Hz, 1H), 4.27-4.19 (m, |
| | | | | | 3H), 2.75 (t, J = 7.8 Hz, 1H), |
| | | | | | 1.67 (s, 3H), 0.82-0.80 (m, |
| | | | | | 3H) ppm. OH missing |

The following compounds were made using a method similar to that described in Example 18, except that 3-methylisoxazol-4-amine was used as the amine in step 3 and different alkylating reagents were used in step 5. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 623 | (2R,3S,4S,5R)-3-(2-((S)- | 559.526 | 560.27 | 2.52 | ¹H NMR (400 MHz, |
| | 2-(2-oxa-6- | | | | Chloroform-d) δ 8.91 (d, J = |
| | azaspiro[3.3]heptan-6- | | | | 5.5 Hz, 1H), 7.97 (br s, 1H), |
| | yl)propoxy)-3,4- | | | | 7.06 (d, J = 7.3 Hz, 1H), 6.92 |
| | difluorophenyl)-4,5- | | | | (q, J = 8.5 Hz, 1H), 5.04 (d, |
| | dimethyl-N-(3- | | | | J = 10.5 Hz, 1H), 4.66 (s, 4H), |
| | methylisoxazol-4-yl)-5- | | | | 4.16-4.12 (m, 1H), 4.08 (s, |
| | (trifluoromethyl)tetra- | | | | 1H), 3.77 (s, 1H), 3.35 (s, |
| | hydrofuran-2- | | | | 4H), 2.76 (d, J = 6.4 Hz, 1H), |
| | carboxamide | | | | 2.48 (d, J = 5.0 Hz, 1H), 2.33 |
| | | | | | (s, 3H), 1.65 (s, 3H), 1.04 (d, |
| | | | | | J = 6.0 Hz, 3H), 0.79-0.77 |
| | | | | | (m, 3H) ppm. |
| 624 | (2R,3S,4S,5R)-3-(2-((R)- | 559.526 | 560.29 | 2.46 | ¹H NMR (400 MHz, |
| | 2-(2-oxa-6- | | | | Chloroform-d) δ 8.90 (s, 1H), |
| | azaspiro[3.3]heptan-6- | | | | 7.97 (s, 1H), 7.06 (t, J = 6.4 |
| | yl)propoxy)-3,4- | | | | Hz, 1H), 6.91 (q, J = 8.5 Hz, |
| | difluorophenyl)-4,5- | | | | 1H), 5.02 (d, J = 11.0 Hz, |
| | dimethyl-N-(3- | | | | 1H), 4.71 (s, 4H), 4.06 (t, J = |
| | methylisoxazol-4-yl)-5- | | | | 8.2 Hz, 1H), 3.94-3.90 (m, |
| | (trifluoromethyl)tetra- | | | | 2H), 3.38 (s, 4H), 2.87 (qt, |
| | hydrofuran-2- | | | | J = 7.6 Hz, 1H), 2.45 (br s, |
| | carboxamide | | | | 1H), 2.33 (s, 3H), 1.64 (s, |
| | | | | | 3H), 1.05 (d, J = 6.4 Hz, 3H), |
| | | | | | 0.78 (dd, J = 7.6, 2.1 Hz, 3H) |

The following compounds were made using a method similar to that described in Example 18, except that 3-methylisoxazol-4-amine was used as the amine in step 3 and different amines were used in step 6. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 625 | (2R,3S,4S,5R)-3-(3,4- | 521.453 | 522.7 | 3.4 | ¹H NMR (500 MHz, |
| | difluoro-2-(2-(3- | | | | DMSO-d₆) δ 10.03 (s, 1H), |
| | fluoroazetidin-1- | | | | 9.03 (s, 1H), 7.19-7.10 (m, |
| | yl)ethoxy)phenyl)-4,5- | | | | 2H), 5.18 (d, J = 10.8 Hz, |
| | dimethyl-N-(3- | | | | 1H), 5.12 (dp, J = 57.7, 5.1 |
| | methylisoxazol-4-yl)-5- | | | | Hz, 1H), 4.37 (dd, J = 10.8, |
| | (trifluoromethyl)tetra- | | | | 7.2 Hz, 1H), 4.14-4.10 (m, |
| | hydrofuran-2-carboxamide | | | | 1H), 4.02-3.97 (m, 1H), |
| | | | | | 3.63-3.55 (m, 2H), 3.24- |
| | | | | | 3.09 (m, 2H), 2.90-2.83 |
| | | | | | (m, 1H), 2.83-2.73 (m, |
| | | | | | 2H), 2.22 (s, 3H), 1.62 (s, |
| | | | | | 3H), 0.70 (d, J = 6.2 Hz, |
| | | | | | 3H) ppm. |
| 626 | (2R,3S,4S,5R)-3-(3,4- | 533.488 | 533.7 | 2.98 | ¹H NMR (500 MHz, |
| | difluoro-2-(2-(3- | | | | DMSO-d₆) δ 10.03 (s, 1H), |
| | hydroxy-3- | | | | 9.03 (s, 1H), 7.17-7.10 (m, |
| | methylazetidin-1- | | | | 2H), 5.19 (d, J = 10.7 Hz, |
| | yl)ethoxy)phenyl)-4,5- | | | | 1H), 5.09 (s, 1H), 4.31 (dd, |
| | dimethyl-N-(3- | | | | J = 10.7, 7.3 Hz, 1H), 4.13- |
| | methylisoxazol-4-yl)-5- | | | | 4.07 (m, 1H), 4.06-4.01 |
| | (trifluoromethyl)tetra- | | | | (m, 1H), 3.23-3.19 (m, |
| | hydrofuran-2-carboxamide | | | | 2H), 2.93-2.85 (m, 3H), |
| | | | | | 2.74 (t, J = 5.2 Hz, 2H), |
| | | | | | 2.22 (s, 3H), 1.61 (s, 3H), |
| | | | | | 1.28 (s, 3H), 0.70 (d, J = |
| | | | | | 6.2 Hz, 3H) ppm. |
| 627 | (2R,3S,4S,5R)-3-(3,4- | 533.488 | 535 | 3.32 | ¹H NMR (500 MHz, |
| | difluoro-2-(2-(3- | | | | DMSO-d₆) δ 10.01 (s, 1H), |
| | methoxyazetidin-1- | | | | 9.03 (s, 1H), 7.18-7.11 (m, |
| | yl)ethoxy)phenyl)-4,5- | | | | 2H), 5.18 (d, J = 10.7 Hz, |
| | dimethyl-N-(3- | | | | 1H), 4.39 (dd, J = 10.7, 7.2 |
| | methylisoxazol-4-yl)-5- | | | | Hz, 1H), 4.13-4.09 (m, |
| | (trifluoromethyl)tetra- | | | | 1H), 4.00-3.96 (m, 1H), |
| | hydrofuran-2-carboxamide | | | | 3.90 (p, J = 5.8 Hz, 1H), |
| | | | | | 3.55-3.50 (m, 2H), 3.11 (s, |
| | | | | | 3H), 2.92-2.87 (m, 1H), |
| | | | | | 2.86 (dd, J = 7.8, 5.8 Hz, |
| | | | | | 1H), 2.80 (dd, J = 7.8, 5.8 |
| | | | | | Hz, 1H), 2.77-2.67 (m, |
| | | | | | 2H), 2.23 (s, 3H), 1.63 (s, |
| | | | | | 3H), 0.70 (d, J = 6.1 Hz, |
| | | | | | 3H) ppm. |
| 628 | (2R,3S,4S,5R)-3-(2-(2-(3- | 569.469 | 570.1 | 3.53 | ¹H NMR (500 MHz, |
| | (difluoromethoxy)azetidin- | | | | DMSO-d₆) 8 10.05 (s, 1H), |
| | 1-yl)ethoxy)-3,4- | | | | 9.02 (s, 1H), 7.19-7.11 (m, |
| | difluorophenyl)-4,5- | | | | 2H), 6.66 (t, J = 75.3 Hz, |
| | dimethyl-N-(3- | | | | 1H), 5.19 (d, J = 10.7 Hz, |
| | methylisoxazol-4-yl)-5- | | | | 1H), 4.68 (p, J = 5.9 Hz, |
| | (trifluoromethyl)tetra- | | | | 1H), 4.40 (dd, J = 10.7, 7.4 |
| | hydrofuran-2-carboxamide | | | | Hz, 1H), 4.16-4.10 (m, |
| | | | | | 1H), 4.01-3.96 (m, 1H), |
| | | | | | 3.64-3.57 (m, 2H), 3.10- |
| | | | | | 3.06 (m, 1H), 3.04-3.00 |
| | | | | | (m, 1H), 2.87 (dq, J = 7.4 |
| | | | | | Hz, 1H), 2.82-2.70 (m, |
| | | | | | 2H), 2.23 (s, 3H), 1.64 (s, |
| | | | | | 3H), 0.70 (d, J = 6.2 Hz, |
| | | | | | 3H) ppm. |
| 629 | (2R,3S,4S,5R)-3-(2-(2-(3- | 553.47 | 554.9 | 3.5 | ¹H NMR (500 MHz, |
| | (difluoromethyl)azetidin- | | | | DMSO-d₆) δ 10.07 (s, 1H), |
| | 1-yl)ethoxy)-3,4- | | | | 9.01 (s, 1H), 7.18-7.11 (m, |
| | difluorophenyl)-4,5- | | | | 2H), 6.20 (td, J = 56.9, 5.3 |
| | dimethyl-N-(3- | | | | Hz, 1H), 5.19 (d, J = 10.7 |
| | methylisoxazol-4-yl)-5- | | | | Hz, 1H), 4.40 (dd, J = 10.7, |
| | (trifluoromethyl)tetrahy | | | | 7.2 Hz, 1H), 4.15-4.10 (m, |
| | drofuran-2-carboxamide | | | | 1H), 4.00-3.95 (m, 1H), |
| | | | | | 3.36-3.27 (m, 2H), 3.10 |
| | | | | | (dt, J = 11.0, 6.7 Hz, 2H), |
| | | | | | 2.89-2.79 (m, 2H), 2.78- |
| | | | | | 2.66 (m, 2H), 2.22 (s, 3H), |
| | | | | | 1.64 (s, 3H), 0.70 (d, J = |
| | | | | | 6.3 Hz, 3H) ppm. |
| 630 | (2R,3S,4S,5R)-3-(3,4- | 535.479 | 536.6 | 3.52 | ¹H NMR (500 MHz, |
| | difluoro-2-(2-(3-fluoro- | | | | DMSO-d₆) 8 10.04 (s, 1H), |
| | 3-methylazetidin-1- | | | | 9.03 (s, 1H), 7.18-7.11 (m, |
| | yl)ethoxy)phenyl)-4,5- | | | | 2H), 5.18 (d, J = 10.7 Hz, |
| | dimethyl-N-(3- | | | | 1H), 4.33 (dd, J = 10.7, 7.4 |
| | methylisoxazol-4-yl)-5- | | | | Hz, 1H), 4.15-4.10 (m, |
| | (trifluoromethyl)tetrahy | | | | 1H), 4.06-4.02 (m, 1H), |
| | drofuran-2-carboxamide | | | | 3.36-3.28 (m, 2H), 3.27- |
| | | | | | 3.16 (m, 2H), 2.89 (qd, J = |
| | | | | | 7.4 Hz, 7.4 Hz 1H), 2.80 (t, |
| | | | | | J = 5.2 Hz, 2H), 2.22 (s, |
| | | | | | 3H), 1.62 (s, 3H), 1.47 (d, |
| | | | | | J = 22.4 Hz, 3H), 0.70 (d, |
| | | | | | J = 6.2 Hz, 3H) ppm. |
| 631 | (2R,3S,4S,5R)-3-(3,4- | 519.462 | 519.9 | 2.98 | ¹H NMR (500 MHz, |
| | difluoro-2-(2-(oxetan-3- | | | | DMSO-d₆) δ 10.03 (s, 1H), |
| | ylamino)ethoxy)phenyl)- | | | | 9.03 (s, 1H), 7.19-7.10 (m, |
| | 4,5-dimethyl-N-(3- | | | | 2H), 5.18 (d, J = 10.7 Hz, |
| | methylisoxazol-4-yl)-5- | | | | 1H), 4.62 (t, J = 6.8 Hz, |
| | (trifluoromethyl)tetra- | | | | 2H), 4.36-4.30 (m, 3H), |
| | hydrofuran-2-carboxamide | | | | 4.15-4.10 (m, 1H), 4.09- |
| | | | | | 4.03 (m, 1H), 3.94-3.88 |
| | | | | | (m, 1H), 2.88 (dq, J = 7.4 |
| | | | | | Hz, 7.4 Hz 1H), 2.84-2.77 |
| | | | | | (m, 2H), 2.62 (s, 1H), 2.23 |
| | | | | | (s, 3H), 1.59 (s, 3H), 0.70 |
| | | | | | (d, J = 6.4 Hz, 3H) ppm. |
| 632 | (2R,3S,4S,5R)-3-(3,4- | 533.488 | 534.1 | 3.1 | ¹H NMR (500 MHz, |
| | difluoro-2-(2-(((R)- | | | | DMSO-d₆) δ 10.03 (s, 1H), |
| | tetrahydrofuran-3- | | | | 9.02 (s, 1H), 7.19-7.10 (m, |
| | yl)amino)ethoxy)phenyl | | | | 2H), 5.19 (d, J = 10.7 Hz, |
| | )-4,5-dimethyl-N-(3- | | | | 1H), 4.31 (dd, J = 10.7, 7.3 |
| | methylisoxazol-4-yl)-5- | | | | Hz, 1H), 4.19-4.09 (m, |
| | (trifluoromethyl)tetra- | | | | 2H), 3.75-3.68 (m, 2H), |
| | hydrofuran-2-carboxamide | | | | 3.63 (td, J = 8.0, 5.7 Hz, |
| | | | | | 1H), 3.39 (dd, J = 8.6, 4.4 |
| | | | | | Hz, 1H), 3.32-3.20 (m, |
| | | | | | 1H), 2.91-2.80 (m, 3H), |
| | | | | | 2.23 (s, 3H), 2.00-1.90 (m, |
| | | | | | 2H), 1.67-1.61 (m, 1H), |
| | | | | | 1.59 (s, 3H), 0.71 (d, J = |
| | | | | | 6.2 Hz, 3H) ppm. |
| 633 | (2R,3S,4S,5R)-3-(2-(2- | 545.499 | 545.9 | 3.31 | ¹H NMR (500 MHz, |
| | (6-oxa-3- | | | | DMSO-d₆) δ 10.06 (s, 1H), |
| | azabicyclo[3.1.1]heptan- | | | | 9.03 (s, 1H), 7.17-7.10 (m, |
| | 3-yl)ethoxy)-3,4- | | | | 2H), 5.19 (d, J = 10.4 Hz, |
| | difluorophenyl)-4,5- | | | | 1H), 4.37-4.32 (m, 3H), |
| | dimethyl-N-(3- | | | | 4.31-4.25 (m, 2H), 3.06 |
| | methylisoxazol-4-yl)-5- | | | | (ddd, J = 13.8, 11.3, 2.0 Hz, |
| | (trifluoromethyl)tetra- | | | | 2H), 2.99-2.88 (m, 2H), |
| | hydrofuran-2-carboxamide | | | | 2.84 (dq, J = 7.5 Hz, 7.4 |
| | | | | | Hz, 1H), 2.79-2.71 (m, |
| | | | | | 1H), 2.65 (dd, J = 13.3, |
| | | | | | 11.3 Hz, 2H), 2.23 (s, 3H), |
| | | | | | 2.04 (d, J = 7.7 Hz, 1H), |
| | | | | | 1.57 (s, 3H), 0.70 (d, J = |
| | | | | | 6.4 Hz, 3H) ppm. |
| 634 | (2R,3S,4S,5R)-3-(3,4- | 559.526 | 559.7 | 3.36 | ¹H NMR (500 MHz, |
| | difluoro-2-(2- | | | | DMSO-d₆) δ 10.04 (s, 1H), |
| | ((3aR,6aS)-tetrahydro- | | | | 9.03 (s, 1H), 7.16-7.11 (m, |
| | 1H-furo[3,4-c]pyrrol- | | | | 2H), 5.19 (d, J = 10.6 Hz, |
| | 5(3H)- | | | | 1H), 4.30 (dd, J = 10.6, 7.4 |
| | yl)ethoxy)phenyl)-4,5- | | | | Hz, 1H), 4.27-4.24 (m, |
| | dimethyl-N-(3- | | | | 1H), 4.20-4.15 (m, 1H), |
| | methylisoxazol-4-yl)-5- | | | | 3.64-3.61 (m, 1H), 3.60- |
| | (trifluoromethyl)tetra- | | | | 3.56 (m, 1H), 3.36 (dd, J = |
| | hydrofuran-2-carboxamide | | | | 8.8, 2.6 Hz, 1H), 3.33- |
| | | | | | 3.27 (m, 1H), 2.88-2.81 |
| | | | | | (m, 1H), 2.72 (t, J = 5.5 Hz, |
| | | | | | 2H), 2.69-2.64 (m, 4H), |
| | | | | | 2.26-2.20 (m, 2H), 2.23 (s, |
| | | | | | 3H), 1.59 (s, 3H), 0.70 (d, |
| | | | | | J = 6.4 Hz, 3H) ppm. |

The following compounds were made using a method similar to that described in Example 18, except that [1,2,4]triazolo[4,3-a]pyridin-6-amine was used as the amine in step 3 and 3-fluoroazetidine was used in step 6. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 635 | (2R,3S,4S,5R)-N- | 557.488 | 558.2 | 3.10 | ¹H NMR (500 MHz, DMSO- |
| | ([1,2,4]triazolo[4,3- | | | | d₆) δ 10.45 (s, 1H), 9.29- |
| | a]pyridin-6-yl)-3-(3,4- | | | | 9.22 (m, 2H), 7.77 (dt, J = |
| | difluoro-2-(2-(3- | | | | 9.8, 0.9 Hz, 1H), 7.40 (dd, J = |
| | fluoroazetidin-1- | | | | 9.8, 1.9 Hz, 1H), 7.20- |
| | yl)ethoxy)phenyl)-4,5- | | | | 7.14 (m, 2H), 5.20-5.10 (m, |
| | dimethyl-5- | | | | 2H), 4.44 (dd, J = 10.7, 7.3 |
| | (trifluoromethyl)tetra- | | | | Hz, 1H), 4.19-4.11 (m, 1H), |
| | hydrofuran-2-carboxamide | | | | 4.00 (dddd, J = 10.8, 5.7, 3.9, |
| | | | | | 2.0 Hz, 1H), 3.65-3.55 (m, |
| | | | | | 2H), 3.25-3.08 (m, 2H), |
| | | | | | 2.92-2.73 (m, 3H), 1.67 (s, |
| | | | | | 3H), 0.75-0.70 (m, 3H) |
| | | | | | ppm. |

The following compounds could be made using methods similar to those described in Example 18, except with different amines in the amide coupling step 3. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 636 | (2R,3S,4S,5R)-3-(2-(2- | 541.51 | 542.7 | 3.08 | ¹H NMR (500 MHz, DMSO- |
| | (2-oxa-6- | | | | d₆) δ 10.39 (s, 1H), 8.77 (d, J = |
| | azaspiro[3.3]heptan-6- | | | | 2.5 Hz, 1H), 8.30 (dd, J = |
| | yl)ethoxy)-3,4- | | | | 4.7, 1.5 Hz, 1H), 8.04 (ddd, J = |
| | difluorophenyl)-4,5- | | | | 8.3, 2.5, 1.5 Hz, 1H), 7.35 |
| | dimethyl-N-(pyridin-3- | | | | (dd, J = 8.3, 4.7 Hz, 1H), |
| | yl)-5- | | | | 7.18-7.12 (m, 2H), 5.11 (d, |
| | (trifluoromethyl)tetra- | | | | J = 10.7 Hz, 1H), 4.56 (d, J = |
| | hydrofuran-2-carboxamide | | | | 6.6 Hz, 2H), 4.54 (d, J = 6.6 |
| | | | | | Hz, 2H), 4.40 (dd, J = 10.7, |
| | | | | | 7.4 Hz, 1H), 4.13-4.08 (m, |
| | | | | | 1H), 3.96-3.91 (m, 1H), |
| | | | | | 3.32 (d, J = 7.2 Hz, 2H), 3.28 |
| | | | | | (d, J = 7.2 Hz, 2H), 2.84 (dq, |
| | | | | | J = 7.4, 7.4 Hz, 1H), 2.67- |
| | | | | | 2.60 (m, 2H), 1.63 (s, 3H), |
| | | | | | 0.69 (d, J = 6.5 Hz, 3H) ppm. |
| 637 | (2R,3S,4S,5R)-3-(2-(2- | 612.588 | 613 | 3.03 | ¹H NMR (500 MHz, DMSO- |
| | (2-oxa-6- | | | | d₆) δ 10.65 (s, 1H), 8.34 (d, J = |
| | azaspiro[3.3]heptan-6- | | | | 5.6 Hz, 1H), 7.73 (s, 1H), |
| | yl)ethoxy)-3,4- | | | | 7.56 (dd, J = 5.7, 1.9 Hz, |
| | difluorophenyl)-4,5- | | | | 1H), 7.22-7.02 (m, 2H), |
| | dimethyl-N-(2-(N- | | | | 5.13 (d, J = 10.8 Hz, 1H), |
| | methylacetamido)pyridin- | | | | 4.65-4.48 (m, 4H), 4.41 (dd, |
| | 4-yl)-5- | | | | J = 10.7, 7.2 Hz, 1H), 4.11 |
| | (trifluoromethyl)tetra- | | | | (dt, J = 10.5, 5.1 Hz, 1H), |
| | hydrofuran-2-carboxamide | | | | 3.95 (dt, J = 10.2, 5.0 Hz, |
| | | | | | 1H), 3.23 (s, 3H), 2.84 (p, J = |
| | | | | | 7.5 Hz, 1H), 2.73-2.57 (m, |
| | | | | | 2H), 2.01 (s, 3H), 1.63 (s, |
| | | | | | 3H), 0.87-0.57 (m, 3H); NB |
| | | | | | water peak obscures 4H |

### Example 19

(2R,3S,4S,5R)-3-(3,4-difluoro-2-hydroxyphenyl)-N-(2-(hydroxymethyl)pyridin-4-yl)-4,5-dimethyl-5-(trifluoromethyl)tetrahydrofuran-2-carboxamide (638) and (2R,3S,4S,5R)-3-(3,4-difluoro-2-(2-methoxyethoxy)phenyl)-N-(2-(hydroxymethyl)pyridin-4-yl)-4,5-dimethyl-5-(trifluoromethyl)tetrahydrofuran-2-carboxamide (639)

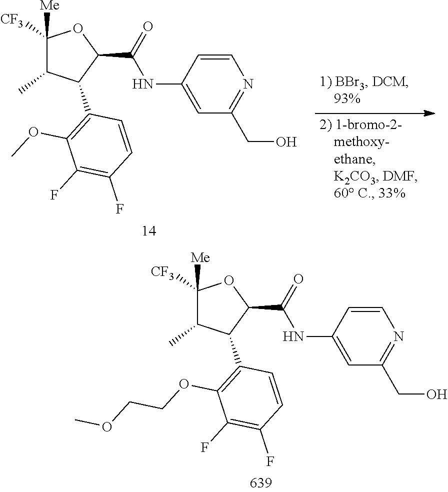 [SMILES: COc1c([C@H]2[C@H](C(=O)Nc3ccnc(CO)c3)O[C@@](C)(C(F)(F)F)[C@H]2C)ccc(F)c1F ; COCCOc1c([C@H]2[C@H](C(=O)Nc3ccnc(CO)c3)O[C@@](C)(C(F)(F)F)[C@H]2C)ccc(F)c1F]

### Step 1:

To a solution of (2R,3S,4S,5R)-3-(3,4-difluoro-2-methoxyphenyl)-N-(2-(hydroxymethyl)pyridin-4-yl)-4,5-dimethyl-5-(trifluoromethyl)tetrahydrofuran-2-carboxamide (14, 820 mg, 1.781 mmol) in DCM (8 mL) at 0° C. was added BBr₃ (5.8 mL of 1 M solution in DCM, 5.8 mmol). The reaction mixture was stirred at ambient temperature overnight. The reaction was cooled to 0° C. and MeOH (4 mL, 98.75 mmol) was added. The reaction mixture was stirred at ambient temperature for 10 min. The reaction mixture was concentrated in vacuo, then dissolved in DCM and saturated aqueous sodium bicarbonate was added. The organic layer was separated, dried (MgSO₄) and concentrated in vacuo to give (2R,3S,4S,5R)-3-(3,4-difluoro-2-hydroxyphenyl)-N-(2-(hydroxymethyl)pyridin-4-yl)-4,5-dimethyl-5-(trifluoromethyl)tetrahydrofuran-2-carboxamide (638, 782 mg, 93%). ESI-MS m/z calc. 446.1265, found 446.9 (M+1)⁺; Retention time: 3.24 minutes.

### Step 2:

To a solution of (2R,3S,4S,5R)-3-(3,4-difluoro-2-hydroxyphenyl)-N-(2-(hydroxymethyl)pyridin-4-yl)-4,5-dimethyl-5-(trifluoromethyl)tetrahydrofuran-2-carboxamide (21 mg, 0.048 mmol) in DMF (500 μL) was added 1-bromo-2-methoxy-ethane (7.5 μL, 0.079 mmol) and K₂CO₃ (7.9 mg, 0.057 mmol). The reaction mixture was heated at 60° C. in a sealed tube. The reaction mixture was filtered and concentrated in vacuo. Purification by reverse phase HPLC-MS using a X-bridge C18 column (150×19 mm, 5 m particle size) from Waters gave (2R,3S,4S,5R)-3-(3,4-difluoro-2-(2-methoxyethoxy)phenyl)-N-(2-(hydroxymethyl)pyridin-4-yl)-4,5-dimethyl-5-(trifluoromethyl)tetrahydrofuran-2-carboxamide (639, 8.2 mg, 33%). ¹H NMR (400 MHz, DMSO-d₆) δ 10.54 (s, 1H), 8.33 (d, J=5.5 Hz, 1H), 7.77-7.69 (m, 1H), 7.50 (dd, J=5.5, 2.1 Hz, 1H), 7.17 (dd, J=9.5, 6.4 Hz, 2H), 5.40 (t, J=5.7 Hz, 1H), 5.11 (d, J=10.7 Hz, 1H), 4.50 (d, J=5.5 Hz, 2H), 4.42-4.17 (m, 3H), 3.71-3.54 (m, 2H), 3.29 (s, 3H), 2.85 (t, J=7.3 Hz, 1H), 1.61 (s, 3H), 0.71 (d, J=7.4 Hz, 3H) ppm. ESI-MS m/z calc. 504.16837, found 505.2 (M+1)⁺; Retention time: 3.15 minutes.

The following compounds were made using the same method as described in Example 19, except that different alkylating agents were used in place of 1-bromo-2-methoxy-ethane in step 2. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 640 | (2R,3S,4S,5R)-3-(2-(2- | 565.553 | 565.7 | 3.43 | ¹H NMR (500 MHz, |
| | ((dimethyl(oxo)-λ⁶- | | | | DMSO-d₆) δ 10.58 (s, 1H), |
| | sulfaneylidene)amino)ethoxy)- | | | | 8.32 (d, J = 5.5 Hz, 1H), |
| | 3,4-difluorophenyl)-N- | | | | 7.71 (d, J = 2.1 Hz, 1H), |
| | (2-(hydroxymethyl)pyridin- | | | | 7.49 (dd, J = 5.5, 2.1 Hz, |
| | 4-yl)-4,5-dimethyl-5- | | | | 1H), 7.17-7.11 (m, 2H), |
| | (trifluoromethyl)tetrahydrofuran- | | | | 5.38 (d, J = 5.5 Hz, 1H), |
| | 2-carboxamide | | | | 5.11 (d, J = 10.5 Hz, 1H), |
| | | | | | 4.49 (d, J = 5.5 Hz, 2H), |
| | | | | | 4.43 (dd, J = 10.5, 7.4 Hz, |
| | | | | | 1H), 4.25-4.19 (m, 1H), |
| | | | | | 4.11-4.05 (m, 1H), 3.30- |
| | | | | | 3.26 (m, 2H), 3.01 (s, 3H), |
| | | | | | 2.98 (s, 3H), 2.92-2.86 (m, |
| | | | | | 1H), 1.63 (s, 3H), 0.72- |
| | | | | | 0.69 (m, 3H) ppm. |
| 641 | (2R,3S,4S,5R)-3-(2-(2- | 540.428 | 541.2 | 3.26 | ¹H NMR (500 MHz, |
| | (difluoromethoxy)ethoxy)- | | | | DMSO-d₆) δ 10.56 (s, 1H), |
| | 3,4-difluorophenyl)-N-(2- | | | | 8.33 (d, J = 5.5 Hz, 1H), |
| | (hydroxymethyl)pyridin-4- | | | | 7.77-7.66 (m, 1H), 7.50 |
| | yl)-4,5-dimethyl-5- | | | | (dd, J = 5.5, 2.1 Hz, 1H), |
| | (trifluoromethyl)tetrahydrofuran- | | | | 7.25-7.09 (m, 2H), 6.76 (t, |
| | 2-carboxamide | | | | J = 75.6 Hz, 1H), 5.39 (s, |
| | | | | | 1H), 5.11 (d, J = 10.6 Hz, |
| | | | | | 1H), 4.50 (d, J = 2.6 Hz, |
| | | | | | 2H), 4.43-4.25 (m, 3H), |
| | | | | | 4.17 (t, J = 4.3 Hz, 2H), |
| | | | | | 2.83 (t, J = 7.4 Hz, 1H), |
| | | | | | 1.59 (s, 3H), 0.78-0.66 (m, |
| | | | | | 3H) ppm. |
| 642 | (2R,3S,4S,5R)-3-(3,4- | 518.474 | 519.3 | 3.23 | ¹H NMR (500 MHz, |
| | difluoro-2-(3- | | | | DMSO-d₆) δ 10.61 (s, 1H), |
| | methoxypropoxy)phenyl)- | | | | 8.33 (d, J = 5.6 Hz, 1H), |
| | N-(2- | | | | 7.71 (d, J = 2.1 Hz, 1H), |
| | (hydroxymethyl)pyridin-4- | | | | 7.50 (dd, J = 5.6, 2.1 Hz, |
| | yl)-4,5-dimethyl-5- | | | | 1H), 7.25-7.10 (m, 2H), |
| | (trifluoromethyl)tetrahydrofuran- | | | | 5.40 (s, 1H), 5.10 (d, J = |
| | 2-carboxamide | | | | 10.4 Hz, 1H), 4.50 (s, 2H), |
| | | | | | 4.37-4.19 (m, 2H), 4.13 |
| | | | | | (dtd, J = 9.4, 6.3, 1.4 Hz, |
| | | | | | 1H), 3.57-3.44 (m, 2H), |
| | | | | | 3.24 (s, 3H), 2.76 (p, J = |
| | | | | | 7.5 Hz, 1H), 1.98 (p, J = |
| | | | | | 6.3 Hz, 2H), 1.61 (s, 3H), |
| | | | | | 0.81-0.68 (m, 3H) ppm. |
| 643 | (2R,3S,4S,5R)-3-(2-(2,2- | 524.429 | 525.2 | 3.32 | ¹H NMR (500 MHz, |
| | difluoropropoxy)-3,4- | | | | DMSO-d₆) δ 10.58 (s, 1H), |
| | difluorophenyl)-N-(2- | | | | 8.34 (d, J = 5.5 Hz, 1H), |
| | (hydroxymethyl)pyridin-4- | | | | 7.73 (d, J = 2.1 Hz, 1H), |
| | yl)-4,5-dimethyl-5- | | | | 7.30-7.12 (m, 2H), 5.41 (s, |
| | (trifluoromethyl)tetrahydrofuran- | | | | 1H), 5.12 (d, J = 10.5 Hz, |
| | 2-carboxamide | | | | 1H), 4.60-4.21 (m, 6H), |
| | | | | | 2.79 (p, J = 7.4 Hz, 1H), |
| | | | | | 1.75 (t, J = 19.3 Hz, 3H), |
| | | | | | 1.58 (s, 3H), 0.79-0.65 (m, |
| | | | | | 3H) ppm. |
| 644 | (2R,3S,4S,5R)-3-(2-((3,3- | 550.466 | 551.2 | 3.48 | ¹H NMR (500 MHz, |
| | difluorocyclobutyl)methoxy)- | | | | DMSO-d₆) δ 10.60 (s, 1H), |
| | 3,4-difluorophenyl)-N- | | | | 8.33 (d, J = 5.5 Hz, 1H), |
| | (2-(hydroxymethyl)pyridin- | | | | 7.70 (d, J = 2.0 Hz, 1H), |
| | 4-yl)-4,5-dimethyl-5- | | | | 7.50 (dd, J = 5.6, 2.1 Hz, |
| | (trifluoromethyl)tetrahydrofuran- | | | | 1H), 7.25-7.11 (m, 2H), |
| | 2-carboxamide | | | | 5.40 (s, 1H), 5.10 (d, J = |
| | | | | | 10.4 Hz, 1H), 4.50 (s, 2H), |
| | | | | | 4.26 (dt, J = 10.3, 8.0 Hz, |
| | | | | | 2H), 4.14 (dd, J = 9.8, 6.5 |
| | | | | | Hz, 1H), 2.74 (pd, J = 7.5, |
| | | | | | 3.6 Hz, 3H), 2.66-2.52 (m, |
| | | | | | 1H), 2.49-2.44 (m, 1H), |
| | | | | | 1.60 (s, 3H), 0.73 (dd, J = |
| | | | | | 7.4, 2.5 Hz, 3H) ppm. |
| 645 | (2R,3S,4S,5R)-3-(3,4- | 534.448 | 535.2 | 3.06 | ¹H NMR (500 MHz, |
| | difluoro-2-((3- | | | | DMSO-d₆) δ 10.58 (s, 1H), |
| | fluorooxetan-3- | | | | 8.39-8.25 (m, 1H), 7.79- |
| | yl)methoxy)phenyl)-N-(2- | | | | 7.69 (m, 1H), 7.49 (dd, J = |
| | (hydroxymethyl)pyridin-4- | | | | 5.6, 2.2 Hz, 1H), 7.28- |
| | yl)-4,5-dimethyl-5- | | | | 7.12 (m, 2H), 5.40 (t, J = |
| | (trifluoromethyl)tetrahydrofuran- | | | | 5.8 Hz, 1H), 5.11 (d, J = |
| | 2-carboxamide | | | | 10.6 Hz, 1H), 4.80-4.53 |
| | | | | | (m, 6H), 4.50 (d, J = 5.8 |
| | | | | | Hz, 2H), 4.25 (dd, J = 10.6, |
| | | | | | 7.3 Hz, 1H), 2.73 (t, J = 7.3 |
| | | | | | Hz, 1H), 1.57 (s, 3H), 0.81- |
| | | | | | 0.66 (m, 3H) ppm. |
| 646 | (2R,3S,4S,5R)-3-(3,4- | 518.474 | 519.2 | 3.36 | ¹H NMR (500 MHz, |
| | difluoro-2-(((R)-1- | | | | DMSO-d₆) δ 10.52 (s, 1H), |
| | methoxypropan-2- | | | | 8.33 (d, J = 5.5 Hz, 1H), |
| | yl)oxy)phenyl)-N-(2- | | | | 7.72 (d, J = 2.0 Hz, 1H), |
| | (hydroxymethyl)pyridin-4- | | | | 7.52 (dd, J = 5.5, 2.1 Hz, |
| | yl)-4,5-dimethyl-5- | | | | 1H), 7.21-7.10 (m, 2H), |
| | (trifluoromethyl)tetrahydrofuran- | | | | 5.40 (s, 1H), 5.11 (d, J = |
| | 2-carboxamide | | | | 10.7 Hz, 1H), 4.50 (s, 3H), |
| | | | | | 4.39 (dd, J = 10.7, 7.2 Hz, |
| | | | | | 1H), 3.51-3.39 (m, 2H), |
| | | | | | 3.20 (s, 3H), 2.80 (p, J = |
| | | | | | 7.4 Hz, 1H), 1.62 (s, 3H), |
| | | | | | 1.33 (d, J = 6.3 Hz, 3H), |
| | | | | | 0.67 (dd, J = 7.3, 2.5 Hz, |
| | | | | | 3H) ppm. |

The following compounds were made using a method similar to that described in Example 19, except that 222 and 385, respectively, were used in place of 14 in step 1. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 647 | (2R,3S,4S,5R)-3-(3,4- | 534.473 | 535 | 0.87 | ¹H NMR (500 MHz, DMSO- |
| | difluoro-2-(2- | | | | d₆) δ 10.54 (s, 1H), 8.34 (d, J = |
| | methoxyethoxy)phenyl)- | | | | 5.5 Hz, 1H), 7.72 (d, J = 2.1 |
| | N-(2-((R)-1,2- | | | | Hz, 1H), 7.53 (dd, J = 5.6, 2.1 |
| | dihydroxyethyl)pyridin- | | | | Hz, 1H), 7.22-7.10 (m, 2H), |
| | 4-yl)-4,5-dimethyl-5- | | | | 5.38 (d, J = 4.7 Hz, 1H), 5.10 |
| | (trifluoromethyl)tetrahydrofuran- | | | | (d, J = 10.7 Hz, 1H), 4.66 (t, J = |
| | 2-carboxamide | | | | 5.9 Hz, 1H), 4.53 (dt, J = |
| | | | | | 6.8, 4.3 Hz, 1H), 4.35 (dd, J = |
| | | | | | 10.7, 7.2 Hz, 1H), 4.31-4.24 |
| | | | | | (m, 1H), 4.21 (dddd, J = 11.2, |
| | | | | | 4.9, 3.0, 1.2 Hz, 1H), 3.72- |
| | | | | | 3.55 (m, 3H), 3.50-3.37 (m, |
| | | | | | 1H), 3.29 (s, 3H), 2.85 (t, J = |
| | | | | | 7.4 Hz, 1H), 1.61 (s, 3H), 0.73- |
| | | | | | 0.66 (m, 3H) ppm. |
| 648 | (2R,3S,4S,5R)-3-(3,4- | 518.474 | 519.3 | 3.16 | ¹H NMR (500 MHz, DMSO- |
| | difluoro-2-(2- | | | | d₆) δ 10.45 (s, 1H), 8.34 (d, J = |
| | methoxyethoxy)phenyl)- | | | | 5.5 Hz, 1H), 7.51 (d, J = 1.9 |
| | N-(2-(2- | | | | Hz, 1H), 7.45 (dd, J = 5.7, 2.0 |
| | hydroxyethyl)pyridin-4- | | | | Hz, 1H), 7.24-7.09 (m, 2H), |
| | yl)-4,5-dimethyl-5- | | | | 5.10 (d, J = 10.7 Hz, 1H), 4.62 |
| | (trifluoromethyl)tetrahydrofuran- | | | | (t, J = 5.3 Hz, 1H), 4.34 (dd, J = |
| | 2-carboxamide | | | | 10.8, 7.2 Hz, 1H), 4.28 (td, J = |
| | | | | | 5.7, 2.9 Hz, 1H), 4.21 (q, J = |
| | | | | | 7.5, 5.3 Hz, 1H), 3.71 (q, J = |
| | | | | | 6.3 Hz, 2H), 3.63 (td, J = 5.1, |
| | | | | | 3.0 Hz, 2H), 3.29 (s, 3H), 2.82 |
| | | | | | (dt, J = 19.1, 7.0 Hz, 3H), 1.61 |
| | | | | | (s, 3H), 0.76-0.63 (m, 3H) |
| | | | | | ppm. |

The following compound was made using a method similar to that described in Example 19, except that methyl 5-[[(2R,3S,4S,5R)-3-(3,4-difluoro-2-methoxy-phenyl)-4,5-dimethyl-5-(trifluoromethyl)tetrahydrofuran-2-carbonyl]amino]pyridine-2-carboxylate (precursor to 1) was used in place of 14 in step 1. Step 2 was omitted and amination using General Method L was used as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 649 | 5-((2R,3S,4S,5R)-3- | 459.367 | 460.2 | 2.76 | ¹H NMR (500 MHz, |
| | (3,4-difluoro-2- | | | | DMSO-d₆) δ 10.66 (s, |
| | hydroxyphenyl)-4,5- | | | | 1H), 10.45 (s, 1H), 8.83 |
| | dimethyl-5- | | | | (d, J = 2.5 Hz, 1H), 8.20 |
| | (trifluoromethyl)tetra- | | | | (dd, J = 8.6, 2.5 Hz, 1H), |
| | hydrofuran-2- | | | | 8.02-7.94 (m, 2H), 7.51 |
| | carboxamido)picolinamide | | | | (d, J = 2.8 Hz, 1H), 7.03 |
| | | | | | (td, J = 6.5, 5.7, 3.3 Hz, |
| | | | | | 1H), 6.85 (q, J = 8.8 Hz, |
| | | | | | 1H), 5.13 (d, J = 10.3 Hz, |
| | | | | | 1H), 4.27 (dd, J = 10.3, |
| | | | | | 7.6 Hz, 1H), 2.84 (p, J = |
| | | | | | 7.5 Hz, 1H), 1.60 (s, 3H), |
| | | | | | 0.74-0.69 (m, 3H) ppm. |

The following compounds were made using a method similar to that described in Example 19, except that 30 was used in place of 14 in step 1 and different alkylation conditions were used in step 2. For step 2, (2R,3S,4S,5R)-3-(3,4-difluoro-2-hydroxy-phenyl)-4,5-dimethyl-N-(3-pyridyl)-5-(trifluoromethyl)tetrahydrofuran-2-carboxamide (100 mg, 0.2402 mmol) and (1,4-dimethylimidazol-2-yl)methanol or (1,5-dimethylimidazol-2-yl)methanol (48 mg, 0.3805 mmol) were dissolved in THF (2.4 mL) before addition of PPh₃ (116 mg, 0.4423 mmol). The resulting mixture was stirred at ambient temperature until all components were dissolved, and then cooled to 0° C. DIAD (80 μL, 0.4063 mmol) was added dropwise at 0° C. and the resulting mixture was stirred at ambient temperature for 2 h. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 650 | (2R,3S,4S,5R)-3-(2- | 524.483 | 525.9 | 3.37 | ¹H NMR (500 MHz, DMSO- |
| | ((1,4-dimethyl-1H- | | | | d₆) δ 10.31 (s, 1H), 8.75 (d, J = |
| | imidazol-2-yl)methoxy)- | | | | 2.6 Hz, 1H), 8.29 (d, J = |
| | 3,4-difluorophenyl)-4,5- | | | | 4.7 Hz, 1H), 8.02 (dd, J = |
| | dimethyl-N-(pyridin-3- | | | | 8.3, 2.6 Hz, 1H), 7.34 (dd, J = |
| | yl)-5- | | | | 8.3, 4.7 Hz, 1H), 7.25- |
| | (trifluoromethyl)tetra- | | | | 7.14 (m, 2H), 6.84 (s, 1H), |
| | hydrofuran-2-carboxamide | | | | 5.19 (d, J = 12.3 Hz, 1H), |
| | | | | | 5.07-5.03 (m, 2H), 4.06 (dd, |
| | | | | | J = 10.7, 7.3 Hz, 1H), 3.63 (s, |
| | | | | | 3H), 2.44-2.38 (m, 1H), |
| | | | | | 1.99 (s, 3H), 1.47 (s, 3H), |
| | | | | | 0.65 (d, J = 6.4 Hz, 3H) ppm. |
| 651 | (2R,3S,4S,5R)-3-(2- | 524.483 | 525.8 | 3.77 | ¹H NMR (500 MHz, DMSO- |
| | ((1,5-dimethyl-1H- | | | | d₆) δ 10.34 (s, 1H), 8.74 (d, J = |
| | imidazol-2-yl)methoxy)- | | | | 2.6 Hz, 1H), 8.29 (dd, J = |
| | 3,4-difluorophenyl)-4,5- | | | | 4.7, 1.5 Hz, 1H), 8.01 (dt, J = |
| | dimethyl-N-(pyridin-3- | | | | 8.3, 2.6, 1.5 Hz, 1H), 7.34 |
| | yl)-5- | | | | (dd, J = 8.3, 4.7 Hz, 1H), |
| | (trifluoromethyl)tetra- | | | | 7.25-7.13 (m, 2H), 6.59 (s, |
| | hydrofuran-2-carboxamide | | | | 1H), 5.21 (d, J = 12.3 Hz, |
| | | | | | 1H), 5.10 (d, J = 12.3 Hz, |
| | | | | | 1H), 5.05 (d, J = 10.5 Hz, |
| | | | | | 1H), 4.04 (dd, J = 10.5, 7.4 |
| | | | | | Hz, 1H), 3.58 (s, 3H), 2.50- |
| | | | | | 2.42 (m, 1H), 2.13 (s, 3H), |
| | | | | | 1.46 (s, 3H), 0.65 (d, J = 7.3 |
| | | | | | Hz, 3H) ppm. |

The following compound was made using a method similar to that described in Example 19, except that (2R,3S,4S,5R)—N-(2-bromo-4-pyridyl)-3-(3,4-difluoro-2-methoxy-phenyl)-4,5-dimethyl-5-(trifluoromethyl)tetrahydrofuran-2-carboxamide (described in Example 11) was used in place of 14 in step 1. The product of step 2 was reacted with (3R)-3-methyl-5-oxo-piperazine-1-carboxylate using conditions described in Example 11, step 2. Deprotection using General Method I and methylation using General Method K were then used as the final steps. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 652 | (2R,3S,4S,5R)-3-(3,4- | 600.577 | 601 | 3.33 | ¹H NMR (500 MHz, DMSO- |
| | difluoro-2-(2- | | | | d₆) δ 10.60 (s, 1H), 8.34 (d, J = |
| | methoxyethoxy)phenyl)- | | | | 5.6 Hz, 1H), 7.96-7.73 (m, |
| | N-(2-((R)-2,4- | | | | 1H), 7.53 (dd, J = 5.6, 1.9 Hz, |
| | dimethyl-6- | | | | 1H), 7.28-7.05 (m, 2H), 5.11 |
| | oxopiperazin-1- | | | | (d, J = 10.7 Hz, 1H), 4.53 (q, J = |
| | yl)pyridin-4-yl)-4,5- | | | | 4.9 Hz, 1H), 4.35 (dd, J = |
| | dimethyl-5- | | | | 10.7, 7.2 Hz, 1H), 4.30-4.26 |
| | (trifluoromethyl)tetra- | | | | (m, 1H), 4.24-4.15 (m, 1H), |
| | hydrofuran-2- | | | | 3.68-3.58 (m, 2H), 3.28 (s, |
| | carboxamide | | | | 3H), 2.94 (d, J = 16.7 Hz, 1H), |
| | | | | | 2.84 (p, J = 7.5 Hz, 1H), 2.71 |
| | | | | | (dd, J = 11.7, 4.2 Hz, 1H), 2.67- |
| | | | | | 2.58 (m, 1H), 2.27 (s, 3H), |
| | | | | | 1.61 (s, 3H), 1.04 (d, J = 6.3 |
| | | | | | Hz, 3H), 0.81-0.63 (m, 3H) |
| | | | | | ppm; water peak obscures 1H. |

The following compound was made using a method similar to that described in Example 19, except that (2R,3S,4S,5R)—N-(2-bromo-4-pyridyl)-3-(3,4-difluoro-2-methoxy-phenyl)-4,5-dimethyl-5-(trifluoromethyl)tetrahydrofuran-2-carboxamide (described in Example 11) was used in place of 14 in step 1. 2-bromoethan-1-ol was used as the alkylating agent in step 2 and the product of step 2 was reacted with (6R)-4,6-dimethylpiperazin-2-one using conditions described in Example 11, step 2 as the final step. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 653 | (2R,3S,4S,5R)-3-(3,4- | 586.551 | 587.2 | 2.22 | ¹H NMR (500 MHz, |
| | difluoro-2-(2- | | | | Chloroform-d) δ 8.64 (s, 1H), |
| | hydroxyethoxy)phenyl)- | | | | 8.30 (d, J = 5.5 Hz, 1H), 7.73 |
| | N-(2-((R)-2,4-dimethyl- | | | | (s, 1H), 7.32 (d, J = 5.5 Hz, |
| | 6-oxopiperazin-1- | | | | 1H), 7.05 (t, J = 7.1 Hz, 1H), |
| | yl)pyridin-4-yl)-4,5- | | | | 6.91 (q, J = 8.4 Hz, 1H), 4.90 |
| | dimethyl-5- | | | | (d, J = 11.0 Hz, 1H), 4.71 (t, |
| | (trifluoromethyl)tetra- | | | | J = 5.3 Hz, 1H), 4.51-4.46 |
| | hydrofuran-2-carboxamide | | | | (m, 1H), 4.35-4.30 (m, 1H), |
| | | | | | 4.20-4.15 (m, 1H), 3.80 (t, J = |
| | | | | | 4.4 Hz, 2H), 3.42 (d, J = |
| | | | | | 16.9 Hz, 1H), 3.14 (d, J = |
| | | | | | 16.0 Hz, 2H), 2.84 (d, J = 9.6 |
| | | | | | Hz, 1H), 2.77-2.69 (m, 2H), |
| | | | | | 2.41 (s, 3H), 1.65 (s, 3H), |
| | | | | | 1.15 (t, J = 6.9 Hz, 3H), 0.83- |
| | | | | | 0.78 (m, 3H) ppm. |

### Example 20

(2R,3S,4S,5R)-3-(3,4-difluoro-2-((1S,3R)-3-hydroxycyclobutoxy)phenyl)-4,5-dimethyl-N-(pyridin-3-yl)-5-(trifluoromethyl)tetrahydrofuran-2-carboxamide (654)

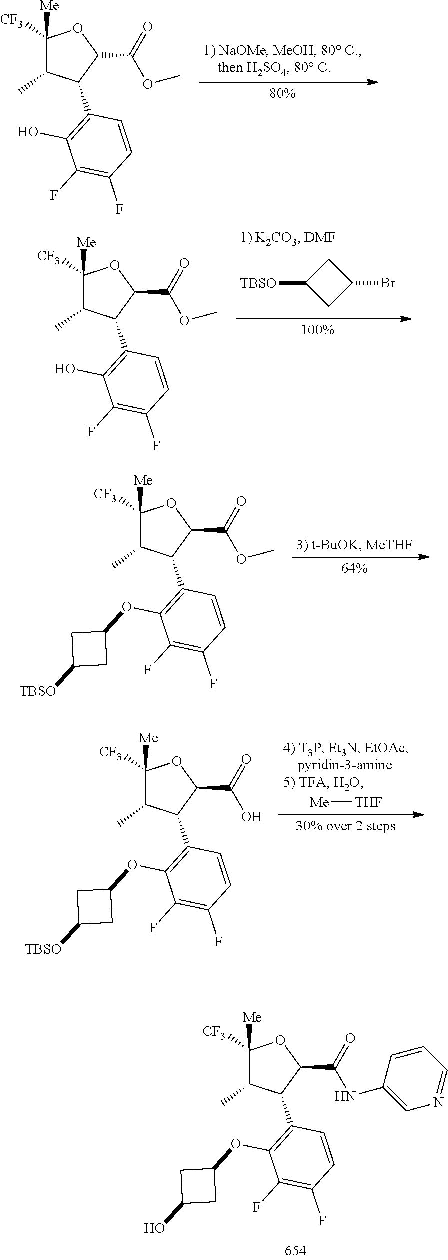

### Step 1:

To a solution of methyl (2S,3S,4S,5R)-3-(3,4-difluoro-2-hydroxy-phenyl)-4,5-dimethyl-5-(trifluoromethyl)tetrahydrofuran-2-carboxylate (5 g, 14.11 mmol) in MeOH (25 mL) was added methanolate (Sodium salt) (13 mL of 25% w/w solution in MeOH, 56.85 mmol) at ambient temperature. The reaction mixture was heated at 80° C. for 1 hour, then cooled down to ambient temperature, and concentrated H₂SO₄ (3.2 mL, 60.03 mmol) was added. The reaction mixture was heated at 80° C. for 10 min, then cooled to ambient temperature and partitioned between DCM and water. The organic extract was dried (MgSO₄) and concentrated in vacuo to give methyl (2R,3S,4S,5R)-3-(3,4-difluoro-2-hydroxy-phenyl)-4,5-dimethyl-5-(trifluoromethyl)tetrahydrofuran-2-carboxylate (4 g, 80%). ESI-MS m/z calc. 354.08905, found 353.0 (M−1)⁻; Retention time: 0.86 minutes.

### Step 2:

To a solution of methyl (2R,3S,4S,5R)-3-(3,4-difluoro-2-hydroxy-phenyl)-4,5-dimethyl-5-(trifluoromethyl)tetrahydrofuran-2-carboxylate (100 mg, 0.2823 mmol) in DMF (1 mL) was added (3-bromocyclobutoxy)-tert-butyl-dimethyl-silane (100 mg, 0.377 mmol) and K₂CO₃ (100 mg, 0.723 mmol). The reaction was heated at 90° C. under a nitrogen atmosphere for 1.5 hours. The reaction mixture was diluted with EtOAc and washed with a saturated sodium bicarbonate aqueous solution and brine. The organic layer was dried (MgSO₄) and concentrated in vacuo to give methyl (2R,3S,4S,5R)-3-[2-[3-[tert-butyl(dimethyl)silyl]oxycyclobutoxy]-3,4-difluoro-phenyl]-4,5-dimethyl-5-(trifluoromethyl)tetrahydrofuran-2-carboxylate (160 mg, 105%) as a beige oil. ESI-MS m/z calc. 538.2174, found 539.0 (M+1)⁺; 537.0 (M−1)⁻; Retention time: 1.37 minutes

### Step 3:

KO-t-Bu (50 mg, 0.4456 mmol) was added to a solution of rac-methyl (2R*,3S*,4S*,5R*)-3-[2-[3-[tert-butyl(dimethyl)silyl]oxycyclobutoxy]-3,4-difluoro-phenyl]-4,5-dimethyl-5-(trifluoromethyl)tetrahydrofuran-2-carboxylate (160 mg, 0.2971 mmol) in 2-MeTHF (5 mL). The reaction mixture was stirred at ambient temperature for 2 hours. The reaction mixture was diluted with EtOAc and washed with a 1 M HCl solution and brine. The organic layer was dried (MgSO₄) and concentrated in vacuo to give (2R,3S,4S,5R)-3-[2-[3-[tert-butyl(dimethyl)silyl]oxycyclobutoxy]-3,4-difluoro-phenyl]-4,5-dimethyl-5-(trifluoromethyl)tetrahydrofuran-2-carboxylic acid (100 mg, 64%) as colourless gum. ESI-MS m/z calc. 524.2017, found 525.0 (M+1)⁺; Retention time: 0.9 minutes.

### Step 4:

To a solution of (2R,3S,4S,5R)-3-[2-[3-[tert-butyl(dimethyl)silyl]oxycyclobutoxy]-3,4-difluoro-phenyl]-4,5-dimethyl-5-(trifluoromethyl)tetrahydrofuran-2-carboxylic acid (50 mg, 0.095 mmol) in EtOAc (2 mL) was added Et₃N (70 μL, 0.502 mmol), T3P solution (130 μL of 50% w/v in EtOAc, 0.204 mmol) and pyridin-3-amine (20 mg, 0.212 mmol). The reaction mixture was stirred at ambient temperature for 18 h. The reaction mixture was partitioned between EtOAc (30 mL) and saturated sodium bicarbonate aqueous solution (20 mL). The combined organic fractions were washed with brine (10 mL), dried (MgSO₄) and concentrated in vacuo to give (2R,3S,4S,5R)-3-(2-((1s,3R)-3-((tert-butyldimethylsilyl)oxy)cyclobutoxy)-3,4-difluorophenyl)-4,5-dimethyl-N-(pyridin-3-yl)-5-(trifluoromethyl)tetrahydrofuran-2-carboxamide. ESI-MS m/z calc. 600.702, found 601.7 (M+1)⁺; 599.7 (M−1)⁻; Retention time: 1.28 minutes.

### Step 5:

To a solution of (2R,3S,4S,5R)-3-(2-((1s,3R)-3-((tert-butyldimethylsilyl)oxy)cyclobutoxy)-3,4-difluorophenyl)-4,5-dimethyl-N-(pyridin-3-yl)-5-(trifluoromethyl)tetrahydrofuran-2-carboxamide in 2-MeTHF (1 mL) was added TFA (100 μL, 1.298 mmol) and water (50 μL, 2.775 mmol). The reaction mixture was stirred at ambient temperature for 3 h. Purification by preparative reverse phase HPLC-MS using an X-bridge C18 column (150×19 mm, 5 m particle size) from Waters (basic eluent) gave (2R,3S,4S,5R)-3-(3,4-difluoro-2-((1s,3R)-3-hydroxycyclobutoxy)phenyl)-4,5-dimethyl-N-(pyridin-3-yl)-5-(trifluoromethyl)tetrahydrofuran-2-carboxamide (654, 14.4 mg, 30%) as a white solid. ¹H NMR (500 MHz, Methanol-d₄) δ 8.76 (dd, 1H), 8.31 (dd, 1H), 8.14-8.10 (m, 1H), 7.42-7.39 (m, 1H), 7.18-7.14 (m, 1H), 7.00-6.97 (m, 1H), 5.10-5.08 (d, 1H), 4.38 (m, 2H), 3.89-3.86 (m, 1H), 2.86-2.79 (m, 3H), 2.24-2.12 (m, 2H), 1.71 (s, 3H), 0.84-0.81 (m, 3H) ppm. Amide NH or alcohol OH not observed. ESI-MS m/z calc. 486.1578, found 487.6 (M+1)⁺; 485.5 (M−1)⁻; Retention time: 3.06 minutes.

The following compounds were made using a method similar to that described in Example 20, except that different amines were used in step 4. In the Table below, “MS r.t.” stands for Mass Spec

| Cmpd | | LC/MS | Found | MS | |
|---|---|---|---|---|---|
| No. | Compound Name | (m/z calc.) | M + 1 | r.t. | NMR (shifts in ppm) |
| 655 | 5-((2R,3S,4S,5R)-3-(3,4- | 529.456 | 530.6 | 3 | ¹H NMR (500 MHz, |
| | difluoro-2-((1S,3R)-3- | | | | Methanol-d₄) δ 8.87 (dd, |
| | hydroxycyclobutoxy)phenyl)- | | | | 1H), 8.23-8.21 (dd, 1H), |
| | 4,5-dimethyl-5- | | | | 8.08-8.06 (d, 1H), 7.14 (m, |
| | (trifluoromethyl)tetrahydrofuran-2- | | | | 1H), 7.00-6.97 (m, 1H), |
| | carboxamido)picolinamide | | | | 5.13-5.10 (d, 1H), 4.38- |
| | | | | | 4.35 (m, 2H), 3.89-3.86 (m, |
| | | | | | 1H), 2.86-2.79 (m, 3H), |
| | | | | | 2.24-2.12 (m, 2H), 1.71 (s, |
| | | | | | 3H), 0.84-0.81 (m, 3H) |
| | | | | | ppm. (Exchangeable NH/ |
| | | | | | OH not observed). |
| 656 | (2R,3S,4S,5R)-3-(3,4- | 490.42 | 491.6 | 3.15 | ¹H NMR (500 MHz, |
| | difluoro-2-((1S,3R)-3- | | | | Methanol-d₄) δ 8.47 (d, |
| | hydroxycyclobutoxy)phenyl)- | | | | 1H), 7.18-7.14 (m, 1H), |
| | 4,5-dimethyl-N-(5- | | | | 7.01-6.96 (m, 1H), 5.10- |
| | methylisoxazol-4-yl)-5- | | | | 5.08 (d, 1H), 4.38-4.32 (m, |
| | (trifluoromethyl)tetra- | | | | 2H), 3.93-3.88 (m, 1H), |
| | hydrofuran-2-carboxamide | | | | 2.87-2.79 (m, 3H), 2.34 (d, |
| | | | | | 3H), 2.22-2.12 (m, 2H), |
| | | | | | 1.71 (s, 3H), 0.84-0.81 (m, |
| | | | | | 3H) ppm. (Exchangeable |
| | | | | | NH/OH not observed). |
| 657 | (2R,3S,4S,5R)-3-(3,4- | 534.448 | 535.6 | 3.34 | |
| | difluoro-2-((1S,3R)-3- | | | | |
| | hydroxycyclobutoxy)phenyl)- | | | | |
| | N-(5-fluoro-2- | | | | |
| | (hydroxymethyl)pyridin-4- | | | | |
| | yl)-4,5-dimethyl-5- | | | | |
| | (trifluoromethyl)tetra- | | | | |
| | hydrofuran-2-carboxamide | | | | |
| 658 | (2R,3S,4S,5R)-3-(3,4- | 487.42 | 488.5 | 2.96 | |
| | difluoro-2-((1s,3R)-3- | | | | |
| | hydroxycyclobutoxy)phenyl)- | | | | |
| | 4,5-dimethyl-N- | | | | |
| | (pyridazin-4-yl)-5- | | | | |
| | (trifluoromethyl)tetra- | | | | |
| | hydrofuran-2-carboxamide | | | | |
| 659 | (2R,3S,4S,5R)-N-(5- | 511.441 | 512.6 | 3.23 | |
| | cyanopyridin-3-yl)-3-(3,4- | | | | |
| | difluoro-2-((1S,3R)-3- | | | | |
| | hydroxycyclobutoxy)phenyl)- | | | | |
| | 4,5-dimethyl-5- | | | | |
| | (trifluoromethyl)tetra- | | | | |
| | hydrofuran-2-carboxamide | | | | |
| 660 | (2R,3S,4S,5R)-3-(3,4- | 504.422 | 505.5 | 3.25 | |
| | difluoro-2-((1S,3R)-3- | | | | |
| | hydroxycyclobutoxy)phenyl)- | | | | |
| | N-(5-fluoropyridin-3-yl)- | | | | |
| | 4,5-dimethyl-5- | | | | |
| | (trifluoromethyl)tetra- | | | | |
| | hydrofuran-2-carboxamide | | | | |
| 661 | (2R,3S,4S,5R)-3-(3,4- | 500.458 | 501.6 | 3.14 | |
| | difluoro-2-((1S,3R)-3- | | | | |
| | hydroxycyclobutoxy)phenyl)- | | | | |
| | 4,5-dimethyl-N-(6- | | | | |
| | methylpyridin-3-yl)-5- | | | | |
| | (trifluoromethyl)tetra- | | | | |
| | hydrofuran-2-carboxamide | | | | |
| 662 | (2R,3S,4S,5R)-N- | 526.456 | 527.55 | 2.875 | ¹H NMR (500 MHz, |
| | ([1,2,4]triazolo[4,3- | | | | Methanol-d₄) δ 9.05 (s, |
| | a]pyridin-7-yl)-3-(3,4- | | | | 1H), 8.44-8.39 (m, 1H), |
| | difluoro-2-((1S,3R)-3- | | | | 8.25-8.21 (m, 1H), 7.20 |
| | hydroxycyclobutoxy)phenyl)- | | | | (dd, J = 7.5, 2.0 Hz, 1H), |
| | 4,5-dimethyl-5- | | | | 7.15-7.09 (m, 1H), 6.97 |
| | (trifluoromethyl)tetra- | | | | (q, J = 8.8 Hz, 1H), 5.09 (d, |
| | hydrofuran-2-carboxamide | | | | J = 10.7 Hz, 1H), 4.41- |
| | | | | | 4.29 (m, 2H), 3.86 (p, J = |
| | | | | | 7.1 Hz, 1H), 2.88-2.76 (m, |
| | | | | | 3H), 2.15 (ddt, J = 26.2, |
| | | | | | 11.4, 7.5 Hz, 2H), 1.69 (s, |
| | | | | | 3H), 0.82 (dd, J = 7.7, 2.3 |
| | | | | | Hz, 3H) ppm. |
| 663 | (2R,3S,4S,5R)-3-(3,4- | 516.458 | 517.6 | 2.9 | |
| | difluoro-2-((1S,3R)-3- | | | | |
| | hydroxycyclobutoxy)phenyl)- | | | | |
| | 4,5-dimethyl-N-(1- | | | | |
| | methyl-2-oxo-1,2- | | | | |
| | dihydropyridin-4-yl)-5- | | | | |
| | (trifluoromethyl)tetra- | | | | |
| | hydrofuran-2-carboxamide | | | | |
| 664 | (2R,3S,4S,5R)-3-(3,4- | 530.484 | 531.6 | 3.16 | |
| | difluoro-2-((1S,3R)-3- | | | | |
| | hydroxycyclobutoxy)phenyl)- | | | | |
| | N-(2- | | | | |
| | (methoxymethyl)pyridin-4- | | | | |
| | yl)-4,5-dimethyl-5- | | | | |
| | (trifluoromethyl)tetra- | | | | |
| | hydrofuran-2-carboxamide | | | | |
| 665 | (2R,3S,4S,5R)-3-(3,4- | 500.458 | 501.6 | 3.18 | ¹H NMR (500 MHz, |
| | difluoro-2-((1S,3R)-3- | | | | Methanol-d₄) δ 8.50 (d, J = |
| | hydroxycyclobutoxy)phenyl)- | | | | 2.4 Hz, 1H), 8.10-8.06 (m, |
| | 4,5-dimethyl-N-(5- | | | | 1H), 7.88 (t, J = 2.3 Hz, |
| | methylpyridin-3-yl)-5- | | | | 1H), 7.07 (ddd, J = 8.3, 5.5, |
| | (trifluoromethyl)tetra- | | | | 2.1 Hz, 1H), 6.91 (ddd, J = |
| | hydrofuran-2-carboxamide | | | | 9.9, 8.9, 7.5 Hz, 1H), 5.01 |
| | | | | | (d, J = 10.7 Hz, 1H), 4.34- |
| | | | | | 4.23 (m, 2H), 3.89-3.74 |
| | | | | | (m, 1H), 2.84-2.67 (m, |
| | | | | | 3H), 2.28 (s, 3H), 2.09 (ddt, |
| | | | | | J = 29.9, 11.5, 7.6 Hz, 2H), |
| | | | | | 1.65-1.61 (m, 3H), 0.76 |
| | | | | | (dq, J = 7.4, 2.3 Hz, 3H) |
| | | | | | ppm. |
| 666 | (2R,3S,4S,5R)-3-(3,4- | 553.499 | | 3.19 | ¹H NMR (500 MHz, |
| | difluoro-2-((1S,3R)-3- | | | | Methanol-d₄) δ 8.41 (d, J = |
| | hydroxycyclobutoxy)phenyl)- | | | | 0.7 Hz, 1H), 8.00 (d, J = 0.7 |
| | 4,5-dimethyl-N-(1- | | | | Hz, 1H), 7.13 (ddd, J = 8.4, |
| | (methylsulfonyl)-1H- | | | | 5.7, 2.1 Hz, 1H), 6.96 (td, J = |
| | pyrazol-4-yl)-5- | | | | 9.3, 7.5 Hz, 1H), 5.05 (d, |
| | (trifluoromethyl)tetra- | | | | J = 10.8 Hz, 1H), 4.33 (pd, |
| | hydrofuran-2-carboxamide | | | | J = 7.2, 2.1 Hz, 1H), 4.26 |
| | | | | | (dd, J = 10.8, 7.8 Hz, 1H), |
| | | | | | 3.85 (p, J = 7.1 Hz, 1H), |
| | | | | | 3.33 (s, 3H), 2.88-2.72 (m, |
| | | | | | 3H), 2.12 (ddt, J = 34.1, |
| | | | | | 11.4, 7.5 Hz, 2H), 1.68 (d, J = |
| | | | | | 1.2 Hz, 3H), 0.80 (dq, J = |
| | | | | | 7.4, 2.3 Hz, 3H) ppm. |
| 667 | (2R,3S,4S,5R)-3-(3,4- | 564.522 | 565.6 | 3.11 | ¹H NMR (500 MHz, |
| | difluoro-2-((1S,3R)-3- | | | | Methanol-d₄) δ 9.03 (d, J = |
| | hydroxycyclobutoxy)phenyl)- | | | | 2.3 Hz, 1H), 8.80 (d, J = 2.1 |
| | 4,5-dimethyl-N-(5- | | | | Hz, 1H), 8.70 (t, J = 2.2 Hz, |
| | (methylsulfonyl)pyridin-3- | | | | 1H), 7.14 (ddd, J = 8.1, 5.5, |
| | yl)-5- | | | | 2.1 Hz, 1H), 6.97 (td, J = |
| | (trifluoromethyl)tetra- | | | | 9.4, 7.5 Hz, 1H), 5.12 (d, J = |
| | hydrofuran-2-carboxamide | | | | 10.6 Hz, 1H), 4.40-4.32 |
| | | | | | (m, 2H), 3.86 (p, J = 7.1 |
| | | | | | Hz, 1H), 3.19 (s, 3H), 2.90- |
| | | | | | 2.74 (m, 3H), 2.14 (ddt, J = |
| | | | | | 32.2, 11.5, 7.5 Hz, 2H), |
| | | | | | 1.70 (s, 3H), 0.82 (dq, J = |
| | | | | | 7.4, 2.3 Hz, 3H) ppm. |

### Example 21

(2R,3S,4S,5R)-3-(3,4-difluoro-2-(hydroxymethyl)phenyl)-N-(1-(difluoromethyl)-3-methyl-1H-pyrazol-4-yl)-4,5-dimethyl-5-(trifluoromethyl)tetrahydrofuran-2-carboxamide (668) and (2R,3S,4S,5R)-3-(3,4-difluoro-2-((methyl(tetrahydro-2H-pyran-4-yl)amino)methyl)phenyl)-N-(1-(difluoromethyl)-3-methyl-1H-pyrazol-4-yl)-4,5-dimethyl-5-(trifluoromethyl)tetrahydrofuran-2-carboxamide (669)

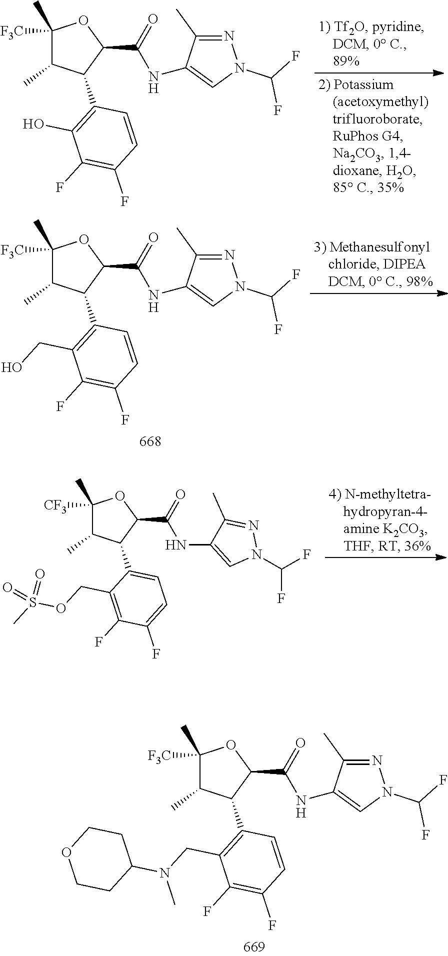

### Step 1:

To a solution of (2R,3S,4S,5R)-3-(3,4-difluoro-2-hydroxy-phenyl)-N-[1-(difluoromethyl)-3-methyl-pyrazol-4-yl]-4,5-dimethyl-5-(trifluoromethyl)tetrahydrofuran-2-carboxamide (1 g, 2.131 mmol) in DCM (10 mL) at 0° C. was added pyridine (350 μL, 4.32 mmol) and trifluoromethylsulfonyl trifluoromethanesulfonate (2.8 mL of 1 M solution in DCM, 2.8 mmol). The reaction mixture was stirred at 0° C. for 20 min. The reaction mixture was warmed to ambient temperature, diluted with DCM (10 mL) and washed with water (20 mL). The organic layer was dried (MgSO₄), filtered and concentrated in vacuo. Purification by silica gel chromatography (SiO₂, 0 to 10% EtOAc in heptane) gave [6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]trifluoromethanesulfonate (1.14 g, 89%). ¹H NMR (400 MHz, Chloroform-d) δ 8.27 (s, 1H), 8.04 (s, 1H), 7.39-7.30 (m, 2H), 7.04 (t, J=60.6 Hz, 1H), 5.04 (d, J=10.9 Hz, 1H), 4.09 (dd, J=10.9, 7.9 Hz, 1H), 2.88 (p, J=7.6 Hz, 1H), 2.30 (d, J=1.1 Hz, 3H), 1.70 (d, J=1.1 Hz, 3H), 0.93 (ddd, J=9.4, 4.3, 2.0 Hz, 3H) ppm. ESI-MS m/z calc. 601.07294, found 602.5 (M+1)⁺; 600.4 (M−1)⁻; Retention time: 1.12 minutes.

### Step 2:

To a solution of [6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]trifluoromethanesulfonate (1.13 g, 1.87 mmol) in 1,4-dioxane (11.5 mL) was added potassium (acetoxymethyl)trifluoroborate (801 mg, 5.686 mmol), [2-(2-aminophenyl)phenyl]-methylsulfonyloxy-palladium; dicyclohexyl-[2-(2,6-diisopropoxyphenyl)phenyl]phosphane (159 mg, 0.1901 mmol) and Na₂CO₃ (997 mg, 9.407 mmol) under a stream of nitrogen. Water (2.3 mL) was added and the reaction mixture heated at 85° C. for 12 h. The mixture was diluted with MTBE (30 mL) and washed with water (30 mL). The aqueous layer was extracted with MTBE (2×30 mL). The combined organic extracts were dried (MgSO₄), filtered and concentrated in vacuo. Purification by flash chromatography (SiO₂, 0 to 50% EtOAc in hexanes) gave (2R,3S,4S,5R)-3-(3,4-difluoro-2-(hydroxymethyl)phenyl)-N-(1-(difluoromethyl)-3-methyl-1H-pyrazol-4-yl)-4,5-dimethyl-5-(trifluoromethyl)tetrahydrofuran-2-carboxamide (668, 314 mg, 35%). ¹H NMR (500 MHz, DMSO-d₆) δ 9.89 (s, 1H), 8.29 (s, 1H), 7.62 (s, 1H), 7.39 (d, J=9.0 Hz, 1H), 7.24 (d, J=8.8 Hz, 1H), 5.21 (d, J=10.6 Hz, 1H), 4.64 (d, J=12.2 Hz, 1H), 4.52 (d, J=12.2 Hz, 1H), 4.41 (dd, J=10.7, 7.4 Hz, 1H), 2.89 (p, J=7.5 Hz, 1H), 2.15 (s, 3H), 1.60 (s, 3H), 0.74 (d, J=7.4 Hz, 3H) ppm. ESI-MS m/z calc. 483.13928, found 484.4 (M+1)⁺; 482.4 (M−1)⁻; Retention time: 3.07 minutes.

### Step 3:

To a solution of (2R,3S,4S,5R)-3-[3,4-difluoro-2-(hydroxymethyl)phenyl]-N-[1-(difluoromethyl)-3-methyl-pyrazol-4-yl]-4,5-dimethyl-5-(trifluoromethyl)tetrahydrofuran-2-carboxamide (254 mg, 0.5255 mmol) in DCM (3.5 mL) at 0° C. was added methanesulfonyl chloride (60 μL, 0.7752 mmol) and DIPEA (150 μL, 1.076 mmol). The reaction mixture was stirred at 0° C. for 15 min. The reaction mixture was diluted with DCM (10 mL) and washed with 1 M NaHCO₃ solution (10 mL). The aqueous layer was extracted with DCM (10 mL). The combined organic extracts were washed with saturated brine solution, dried (MgSO₄), filtered and concentrated in vacuo to give [6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]methyl methanesulfonate (291 mg, 99%). ¹H NMR (400 MHz, Chloroform-d) δ 8.27 (s, 1H), 8.06 (s, 1H), 7.34 (q, J=8.8 Hz, 1H), 7.21-6.87 (m, 2H), 5.34 (dd, J=5.1, 2.1 Hz, 2H), 5.07 (d, J=10.9 Hz, 1H), 4.16-4.10 (m, 1H), 2.96 (s, 3H), 2.82-2.74 (m, 1H), 2.31 (d, J=1.1 Hz, 3H), 1.74 (d, J=1.1 Hz, 3H), 0.90 (dd, J=7.6, 2.5 Hz, 3H) ppm. ESI-MS m/z calc. 561.1168, found 562.5 (M+1)⁺; 560.4 (M−1)⁻; Retention time: 0.95 minutes.

### Step 4:

To a solution of [6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]methyl methanesulfonate (40 mg, 0.07124 mmol) in THF (1 mL) was added potassium carbonate (29 mg, 0.2098 mmol) and N-methyltetrahydropyran-4-amine (17 mg, 0.1476 mmol). The reaction mixture was stirred at ambient temperature for 16 h. The reaction mixture was diluted with MTBE (5 mL), filtered and concentrated in vacuo. Purification by reverse phase HPLC-MS using a X-bridge C18 column (150×19 mm, 5 μm particle size) from Waters gave (2R,3S,4S,5R)-3-(3,4-difluoro-2-((methyl(tetrahydro-2H-pyran-4-yl)amino)methyl)phenyl)-N-(1-(difluoromethyl)-3-methyl-1H-pyrazol-4-yl)-4,5-dimethyl-5-(trifluoromethyl)tetrahydrofuran-2-carboxamide (669, 15 mg, 36%). ¹H NMR (400 MHz, DMSO-d₆) δ 9.81 (s, 1H), 8.28 (s, 1H), 7.64 (t, J=59.2 Hz, 1H), 7.41 (q, J=8.9 Hz, 1H), 7.29 (dd, J=9.0, 4.8 Hz, 1H), 5.19 (d, J=10.8 Hz, 1H), 4.54 (dd, J=10.8, 7.5 Hz, 1H), 3.95-3.87 (m, 2H), 3.69 (s, 2H), 3.25 (td, J=11.6, 5.3 Hz, 1H), 2.95 (t, J=7.4 Hz, 1H), 2.63-2.54 (m, 2H), 2.12 (s, 3H), 2.06 (s, 3H), 1.66 (d, J=12.7 Hz, 3H), 1.60 (s, 3H), 1.58-1.49 (m, 1H), 0.76 (d, J=7.4 Hz, 3H) ppm. ESI-MS m/z calc. 580.22845, found 581.6 (M+1)⁺; 579.6 (M−1)⁻; Retention time: 3.7 minutes.

The following compounds were made using the method described in Example 21 except that different amines were used as coupling partners in Step 4. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 670 | (2R,3S,4S,5R)-3-(2-((2- | 564.496 | 565.6 | 3.37 | ¹H NMR (500 MHz, |
| | oxa-6- | | | | Methanol-d₄) δ 8.24 (s, |
| | azaspiro[3.3]heptan-6- | | | | 1H), 7.51 (d, J = 8.8 Hz, |
| | yl)methyl)-3,4- | | | | 1H), 7.40-7.32 (m, 2H), |
| | difluorophenyl)-N-(1- | | | | 5.19 (d, J = 9.6 Hz, 1H), |
| | (difluoromethyl)-3- | | | | 4.80-4.72 (m, 4H), 4.67 |
| | methyl-1H-pyrazol-4- | | | | (d, J = 14.1 Hz, 1H), 4.58 |
| | yl)-4,5-dimethyl-5- | | | | (d, J = 14.0 Hz, 1H), 4.42 |
| | (trifluoromethyl)tetra- | | | | (s, 4H), 4.30 (t, J = 9.6 |
| | hydrofuran-2-carboxamide | | | | Hz, 1H), 2.90-2.81 (m, |
| | | | | | 1H), 2.26 (s, 3H), 1.69 (s, |
| | | | | | 3H), 0.85 (d, J = 7.7 Hz, |
| | | | | | 3H) ppm. |
| 671 | (2R,3S,4S,5R)-3-(3,4- | 496.422 | 497.5 | 3.29 | ¹H NMR (500 MHz, |
| | difluoro-2- | | | | Methanol-d₄) δ 8.19 (s, |
| | ((methylamino)methyl) | | | | 1H), 7.31 (s, 1H), 7.24 (d, |
| | phenyl)-N-(1- | | | | J = 1.4 Hz, 2H), 5.14 (d, J = |
| | (difluoromethyl)-3- | | | | 10.5 Hz, 1H), 4.36 (dd, |
| | methyl-1H-pyrazol-4- | | | | J = 10.5, 8.2 Hz, 1H), |
| | yl)-4,5-dimethyl-5- | | | | 3.89 (d, J = 13.2 Hz, 1H), |
| | (trifluoromethyl)tetra- | | | | 3.82 (d, J = 13.3 Hz, 1H), |
| | hydrofuran-2-carboxamide | | | | 2.86 (p, J = 7.7 Hz, 1H), |
| | | | | | 2.41 (s, 3H), 2.20 (s, 3H), |
| | | | | | 1.68 (s, 3H), 0.89 (d, J = |
| | | | | | 7.6 Hz, 3H) ppm. |
| 672 | (2R,3S,4S,5R)-N-(1- | 510.448 | 511.55 | 3.805 | ¹H NMR (500 MHz, |
| | (difluoromethyl)-3- | | | | Methanol-d₄) δ 8.20 (s, |
| | methyl-1H-pyrazol-4- | | | | 1H), 7.55 (d, J = 8.9 Hz, |
| | yl)-3-(2- | | | | 1H), 7.41 (d, J = 9.0 Hz, |
| | ((dimethylamino)methyl)- | | | | 1H), 7.33 (s, 1H), 5.23 (d, |
| | 3,4-difluorophenyl)- | | | | J = 9.7 Hz, 1H), 4.65 (d, J = |
| | 4,5-dimethyl-5- | | | | 14.2 Hz, 1H), 4.56 (d, J = |
| | (trifluoromethyl)tetra- | | | | 14.2 Hz, 1H), 4.28 (t, J = |
| | hydrofuran-2-carboxamide | | | | 9.5 Hz, 1H), 3.00 (s, |
| | | | | | 6H), 2.95-2.84 (m, 1H), |
| | | | | | 2.24 (s, 3H), 1.70 (s, 3H), |
| | | | | | 0.85 (d, J = 7.6 Hz, 3H) |
| | | | | | ppm. |
| 673 | rac-(2R,3S,4S,5R)-3- | 538.458 | 539.3 | 0.87 | ¹H NMR (500 MHz, |
| | (3,4-difluoro-2-((3- | | | | Methanol-d₄) δ 8.14 (s, |
| | hydroxyazetidin-1- | | | | 1H), 7.38 (d, J = 9.0 Hz, |
| | yl)methyl)phenyl)-N-(1- | | | | 1H), 7.26 (d, J = 9.0 Hz, |
| | (difluoromethyl)-3- | | | | 1H), 7.20 (s, 1H), 5.10 (d, |
| | methyl-1H-pyrazol-4- | | | | J = 9.8 Hz, 1H), 4.61 (q, J = |
| | yl)-4,5-dimethyl-5- | | | | 14.2 Hz, 3H), 4.39 |
| | (trifluoromethyl)tetra- | | | | (ddd, J = 11.0, 6.4, 2.2 |
| | hydrofuran-2-carboxamide | | | | Hz, 1H), 4.34-4.21 (m, |
| | | | | | 2H), 3.98 (s, 1H), 3.90 |
| | | | | | (dd, J = 11.3, 4.7 Hz, 1H), |
| | | | | | 2.81-2.70 (m, 1H), 2.14 |
| | | | | | (s, 3H), 1.58 (s, 3H), 0.74 |
| | | | | | (d, J = 7.6 Hz, 3H) ppm. |
| 674 | (2R,3S,4S,5R)-3-(3,4- | 565.527 | 567.3 | 3.4 | ¹H NMR (500 MHz, |
| | difluoro-2-((4- | | | | DMSO-d₆) δ 9.87 (s, 1H), |
| | methylpiperazin-1- | | | | 9.50 (s, 1H), 8.29 (s, 1H), |
| | yl)methyl)phenyl)-N-(1- | | | | 7.63 (s, 1H), 7.44 (dt, J = |
| | (difluoromethyl)-3- | | | | 9.0, 2.3 Hz, 1H), 7.29 (d, |
| | methyl-1H-pyrazol-4- | | | | J = 8.8 Hz, 1H), 5.18 (d, J = |
| | yl)-4,5-dimethyl-5- | | | | 10.8 Hz, 1H), 4.48 (dd, |
| | (trifluoromethyl)tetra- | | | | J = 10.9, 7.4 Hz, 1H), |
| | hydrofuran-2-carboxamide | | | | 3.69 (d, J = 13.1 Hz, 1H), |
| | | | | | 3.60 (d, J = 13.1 Hz, 1H), |
| | | | | | 3.39 (s, 1H), 2.90 (dd, J = |
| | | | | | 9.5, 5.6 Hz, 2H), 2.82 (d, |
| | | | | | J = 11.3 Hz, 2H), 2.76 (d, |
| | | | | | J = 3.3 Hz, 4H), 2.37 (dt, |
| | | | | | J = 30.5, 12.2 Hz, 2H), |
| | | | | | 2.12 (s, 3H), 1.61 (s, 3H), |
| | | | | | 0.78 (d, J = 7.4 Hz, 3H) |
| | | | | | ppm. |
| 675 | (2R,3S,4S,5R)-3-(3,4- | 524.475 | 525.2 | 3.97 | ¹H NMR (500 MHz, |
| | difluoro-2-((4- | | | | DMSO-d₆) δ 10.06 (s, |
| | methylpiperazin-1- | | | | 1H), 8.27 (d, J = 5.1 Hz, |
| | yl)methyl)phenyl)-N-(1- | | | | 1H), 7.66 (d, J = 13.4 Hz, |
| | (difluoromethyl)-3- | | | | 2H), 7.38 (d, J = 8.8 Hz, |
| | methyl-1H-pyrazol-4- | | | | 1H), 5.28 (dd, J = 31.0, |
| | yl)-4,5-dimethyl-5- | | | | 9.4 Hz, 1H), 4.67 (d, J = |
| | (trifluoromethyl)tetra- | | | | 14.2 Hz, 1H), 4.31 (dd, J = |
| | hydrofuran-2-carboxamide | | | | 14.3, 8.6 Hz, 1H), 4.19 |
| | | | | | (t, J = 9.7 Hz, 1H), 3.41 |
| | | | | | (d, J = 6.2 Hz, 1H), 3.15 |
| | | | | | (s, 1H), 2.83 (d, J = 19.9 |
| | | | | | Hz, 2H), 2.68 (d, J = 4.3 |
| | | | | | Hz, 2H), 2.16 (s, 3H), |
| | | | | | 1.65 (d, J = 8.4 Hz, 3H), |
| | | | | | 1.36 (t, J = 7.2 Hz, 2H), |
| | | | | | 1.20 (d, J = 7.2 Hz, 1H), |
| | | | | | 0.69 (dd, J = 24.3, 7.4 Hz, |
| | | | | | 3H) ppm. |

### Example 22

(2R,3S,4S,5R)-3-(3,4-difluoro-2-vinylphenyl)-N-(1-(difluoromethyl)-3-methyl-1H-pyrazol-4-yl)-4,5-dimethyl-5-(trifluoromethyl)tetrahydrofuran-2-carboxamide (676) and (2R,3S,4S,5R)-3-(3,4-difluoro-2-(2-hydroxyethyl)phenyl)-N-(1-(difluoromethyl)-3-methyl-1H-pyrazol-4-yl)-4,5-dimethyl-5-(trifluoromethyl)tetrahydrofuran-2-carboxamide (677) and (2R,3S,4S,5R)—N-(1-(difluoromethyl)-3-methyl-1H-pyrazol-4-yl)-3-(2-(2-(dimethylamino)ethyl)-3,4-difluorophenyl)-4,5-dimethyl-5-(trifluoromethyl)tetrahydrofuran-2-carboxamide (678)

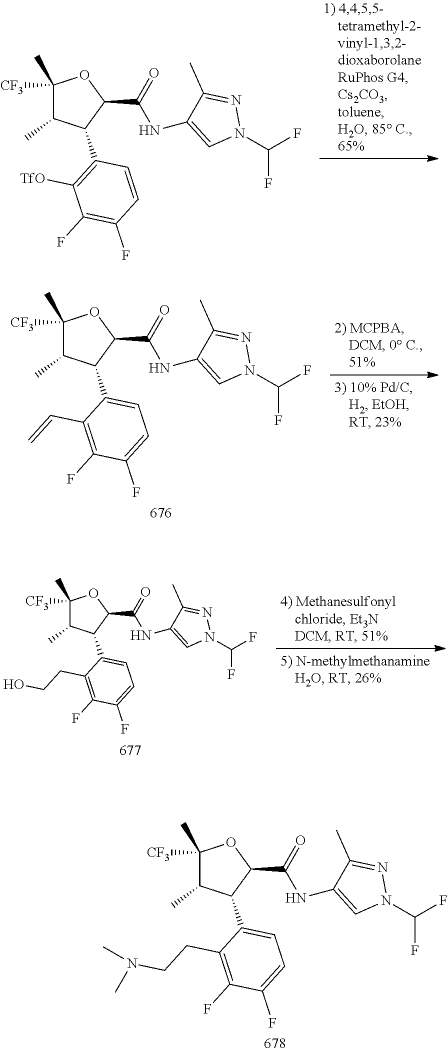 [SMILES: Cc1nn(C(F)F)cc1NC(=O)[C@@H]1O[C@@](C)(C(F)(F)F)[C@@H](C)[C@H]1c1ccc(F)c(F)c1OS(=O)(=O)C(F)(F)F ; C=Cc1c([C@H]2[C@H](C(=O)Nc3cn(C(F)F)nc3C)O[C@@](C)(C(F)(F)F)[C@H]2C)ccc(F)c1F ; Cc1nn(C(F)F)cc1NC(=O)[C@@H]1O[C@@](C)(C(F)(F)F)[C@@H](C)[C@H]1c1ccc(F)c(F)c1CCO ; Cc1nn(C(F)F)cc1NC(=O)[C@@H]1O[C@@](C)(C(F)(F)F)[C@@H](C)[C@H]1c1ccc(F)c(F)c1CCN(C)C]

### Step 1:

To a solution of [6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]trifluoromethanesulfonate (500 mg, 0.831 mmol) in toluene (10 mL) was added 4,4,5,5-tetramethyl-2-vinyl-1,3,2-dioxaborolane (384 mg, 2.49 mmol), Cs₂CO₃ (541 mg, 1.66 mmol), RuPhos G4 (106 mg, 0.124 mmol) and water (2 mL). The reaction mixture was heated at 70° C. for 12 h. The reaction mixture was partitioned between MTBE (20 mL) and water (20 mL). The aqueous layer was extracted with MTBE (10 mL). The combined organic extracts were washed with brine (10 mL), dried (MgSO₄), filtered and concentrated in vacuo. Purification by flash chromatography (SiO₂, 0 to 100% EtOAc in heptane) gave (2R,3S,4S,5R)-3-(3,4-difluoro-2-vinylphenyl)-N-(1-(difluoromethyl)-3-methyl-1H-pyrazol-4-yl)-4,5-dimethyl-5-(trifluoromethyl)tetrahydrofuran-2-carboxamide (676, 300 mg, 65%). ¹H NMR (500 MHz, DMSO-d₆) δ 9.96 (s, 1H), 8.31 (s, 1H), 7.63 (s, 1H), 7.38 (d, J=8.7 Hz, 1H), 7.21 (d, J=8.8 Hz, 1H), 6.79 (dd, J=17.7, 11.6 Hz, 1H), 5.82-5.65 (m, 2H), 5.23 (d, J=10.4 Hz, 1H), 4.22 (dd, J=10.5, 7.5 Hz, 1H), 2.75 (p, J=7.4 Hz, 1H), 2.17 (s, 3H), 1.58 (s, 3H), 0.70 (d, J=7.4 Hz, 3H) ppm. ESI-MS m/z calc. 479.14438, found 480.4 (M+1)⁺; 478.4 (M−1)⁻; Retention time: 3.65 minutes.

### Step 2:

To a solution of (2R,3S,4S,5R)-3-(3,4-difluoro-2-vinylphenyl)-N-(1-(difluoromethyl)-3-methyl-1H-pyrazol-4-yl)-4,5-dimethyl-5-(trifluoromethyl)tetrahydrofuran-2-carboxamide (100 mg, 0.2015 mmol) in DCM (5 mL) was added 3-chloroperbenzoic acid (75 mg, 0.4346 mmol). The reaction mixture was stirred at ambient temperature for 2 days. The reaction mixture was diluted with DCM (5 ml) and washed with a 1:1 mixture of 1M sodium thiosulfate and 1M NaHCO₃. The organic layer was washed with brine (5 mL), dried (MgSO₄), filtered and concentrated in vacuo to give (2R,3S,4S,5R)—N-[1-(difluoromethyl)-3-methyl-pyrazol-4-yl]-3-[3,4-difluoro-2-(oxiran-2-yl)phenyl]-4,5-dimethyl-5-(trifluoromethyl)tetrahydrofuran-2-carboxamide (65 mg, 51%). ESI-MS m/z calc. 495.13928, found 496.2 (M+1)⁺; Retention time: 0.98 and 0.99 minutes.

### Step 3:

To a nitrogen flushed solution of (2R,3S,4S,5R)—N-[1-(difluoromethyl)-3-methyl-pyrazol-4-yl]-3-[3,4-difluoro-2-(oxiran-2-yl)phenyl]-4,5-dimethyl-5-(trifluoromethyl)tetrahydrofuran-2-carboxamide (20 mg, 0.04 mmol) in EtOH (4 mL) was added palladium on carbon (15 mg of 10 wt. % loading, 0.141 mmol). The reaction mixture was stirred under a hydrogen atmosphere for 4 h. The reaction mixture was flushed with nitrogen, filtered and concentrated in vacuo. Purification by reverse phase preparative HPLC (Waters Sunfire C18, 10 μM, 100 A column, 0% to 100% MeCN in water containing 0.1% ammonia) gave (2R,3S,4S,5R)-3-(3,4-difluoro-2-(2-hydroxyethyl)phenyl)-N-(1-(difluoromethyl)-3-methyl-1H-pyrazol-4-yl)-4,5-dimethyl-5-(trifluoromethyl)tetrahydrofuran-2-carboxamide (677, 8 mg, 40%). ¹H NMR (500 MHz, Methanol-d₄) δ 8.18 (d, J=2.0 Hz, 1H), 7.30 (s, 1H), 7.21 (d, J=8.9 Hz, 1H), 7.16 (d, J=8.8 Hz, 1H), 5.18 (d, J=10 Hz, 1H), 4.31 (dd, J=10.6, 8.1 Hz, 1H), 3.70 (dddd, J=30.9, 10.7, 8.1, 6.3 Hz, 2H), 3.06 (ddd, J=14.2, 8.1, 6.4 Hz, 1H), 2.92 (ddd, J=13.9, 8.0, 6.2 Hz, 1H), 2.81 (p, J=7.7 Hz, 1H), 2.19 (d, J=1.0 Hz, 3H), 1.70 (s, 3H), 0.87 (d, J=7.6 Hz, 3H) ppm. ESI-MS m/z calc. 497.15494, found 498.3 (M+1); 496.3 (M−1)⁻; Retention time: 0.9 minutes.

### Step 4:

To a solution of (2R,3S,4S,5R)-3-(3,4-difluoro-2-(2-hydroxyethyl)phenyl)-N-(1-(difluoromethyl)-3-methyl-1H-pyrazol-4-yl)-4,5-dimethyl-5-(trifluoromethyl)tetrahydrofuran-2-carboxamide (150 mg, 0.093 mmol) in DCM (3 mL) was added Et₃N (26 μL, 0.18 mmol) and methanesulfonyl chloride (11 IL, 0.142 mmol). The reaction mixture was stirred at ambient temperature for 20 min. The reaction mixture was partitioned between DCM (10 mL) and 0.5 M NaHCO₃ (5 mL). The aqeuous layer was extracted with DCM (5 mL). The combined organic extracts were washed with brine (10 mL), dried (MgSO₄) and concentrated in vacuo to give 2-[6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]ethyl methanesulfonate (95 mg, 51%). ESI-MS m/z calc. 575.1325, found 576.3 (M+1)⁺; 574.2 (M−1)⁻; Retention time: 0.96 minutes.

### Step 5:

A solution of 2-[6-[(2R,3S,4S,5R)-2-[[1-(difluoromethyl)-3-methyl-pyrazol-4-yl]carbamoyl]-4,5-dimethyl-5-(trifluoromethyl)tetrahydrofuran-3-yl]-2,3-difluoro-phenyl]ethyl methanesulfonate (20 mg, 0.034 mmol) in N-methylmethanamine (870 μL of 40% w/w in water, 6.9 mmol) was stirred at ambient temperature for 3 days. The reaction mixture was concentrated in vacuo. Purification by reverse phase preparative HPLC (Waters Sunfire C18, 10 μM, 100 Å column, 0% to 100% MeCN in water containing 0.1% ammonia) gave (2R,3S,4S,5R)—N-(1-(difluoromethyl)-3-methyl-1H-pyrazol-4-yl)-3-(2-(2-(dimethylamino)ethyl)-3,4-difluorophenyl)-4,5-dimethyl-5-(trifluoromethyl)tetrahydrofuran-2-carboxamide (678, 5 mg, 26%). ¹H NMR (500 MHz, Methanol-d₄) δ 8.05 (s, 1H), 7.15 (s, 1H), 7.09-6.99 (m, 2H), 5.00 (d, J=10.5 Hz, 1H), 4.06 (dd, J=10.6, 8.2 Hz, 1H), 2.96-2.86 (m, 1H), 2.66 (tt, J=15.6, 6.1 Hz, 2H), 2.39 (td, J=12.0, 4.7 Hz, 1H), 2.28 (td, J=12.1, 5.6 Hz, 1H), 2.23 (s, 6H), 2.02 (s, 3H), 1.54 (s, 3H), 0.72 (d, J=7.6 Hz, 3H) ppm. ESI-MS m/z calc. 524.2022, found 526.3 (M+1)⁺; 523.3 (M−1)⁻; Retention time: 0.97 minutes.

The following compound was made using the method described in Example 22 except that 2-oxa-6-azaspiro[3.3]heptane was used displace the mesylate in Step 5. In the Table below, “MS r.t.” stands for Mass Spec retention time.

| Cmpd | | LC/MS | Found | MS | |
|---|---|---|---|---|---|
| No. | Compound Name | (m/z calc.) | M + 1 | r.t. | NMR (shifts in ppm) |
| 679 | (2R,3S,4S,5R)-3-(2-(2- | 578.522 | 579.3 | 3.21 | ¹H NMR (500 MHz, |
| | (2-oxa-6- | | | | Methanol-d₄) δ 8.22 (s, 1H), |
| | azaspiro [3.3]heptan-6- | | | | 7.36-7.22 (m, 3H), 5.20 (d, |
| | yl)ethyl)-3,4- | | | | J = 10.1 Hz, 1H), 4.82 (s, |
| | difluorophenyl)-N-(1- | | | | 4H), 4.52 (s, 2H), 4.29 (s, |
| | (difluoromethyl)-3- | | | | 2H), 4.18 (dd, J = 10.2, 8.6 |
| | methyl-1H-pyrazol-4- | | | | Hz, 1H), 3.42 (ddd, J = 8.5, |
| | yl)-4,5-dimethyl-5- | | | | 6.5, 3.9 Hz, 2H), 3.19-3.11 |
| | (trifluoromethyl)tetra- | | | | (m, 1H), 2.97 (ddd, J = 14.6, |
| | hydrofuran-2-carboxamide | | | | 8.5, 6.3 Hz, 1H), 2.81 (p, J = |
| | | | | | 7.7 Hz, 1H), 2.22 (s, 3H), |
| | | | | | 1.68 (s, 3H), 0.86 (d, J = 7.6 |
| | | | | | Hz, 3H) ppm. |

### Example 23

### rac-(2R*,3S*,4S*,5R*)-3-(3,4-difluoro-2-(1-hydroxyethyl)phenyl)-N-(1-(difluoromethyl)-3-methyl-1H-pyrazol-4-yl)-4,5-dimethyl-5-(trifluoromethyl)tetrahydrofuran-2-carboxamide (680)

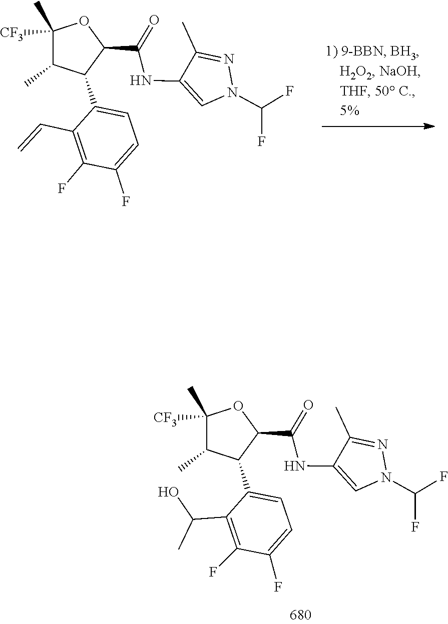 [SMILES: C=Cc1c([C@H]2[C@H](C(=O)Nc3cn(C(F)F)nc3C)O[C@@](C)(C(F)(F)F)[C@H]2C)ccc(F)c1F ; Cc1nn(C(F)F)cc1NC(=O)[C@@H]1O[C@@](C)(C(F)(F)F)[C@@H](C)[C@H]1c1ccc(F)c(F)c1C(C)O]

### Step 1:

To a solution of (2R,3S,4S,5R)—N-[1-(difluoromethyl)-3-methyl-pyrazol-4-yl]-3-(3,4-difluoro-2-vinyl-phenyl)-4,5-dimethyl-5-(trifluoromethyl)tetrahydrofuran-2-carboxamide (20 mg, 0.04 mmol) in THF (1 mL) was added 9-borabicyclo[3.3.1]nonane (241 μL of 0.5 M solution in THF, 0.1205 mmol) and the reaction mixture stirred at ambient temperature for 12 h. Borane (100 μL of 1.0 M solution in THF, 0.1 mmol) was added and the reaction mixture heated at 50° C. for 30 min. A few drops of water were added and the mixture stirred for 15 min followed by the addition of NaOH (403 μL of 1 M aqueous solution, 0.403 mmol) and hydrogen peroxide (45.69 mg of 30% w/w aqueous solution, 0.4 mmol). The reaction mixture was stirred at ambient temperature for 1 h. The reaction mixture was partitioned between MTBE (5 mL) and water (5 mL). The aqueous layer was extracted with MTBE (5 mL). The combined organic extracts were washed with brine (10 mL), dried (MgSO₄) and concentrated in vacuo. Purification by reverse phase HPLC-MS using a X-bridge C18 column (150×19 mm, 5 m particle size) gave (2R*,3S*,4S*,5R*)-3-(3,4-difluoro-2-(1-hydroxyethyl)phenyl)-N-(1-(difluoromethyl)-3-methyl-1H-pyrazol-4-yl)-4,5-dimethyl-5-(trifluoromethyl)tetrahydrofuran-2-carboxamide (680, 1 mg, 5%), as a mixture of epimers at the (1-hydroxyethyl)phenyl group. ¹H NMR (500 MHz, Methanol-d₄) δ 8.18 (s, 1H), 7.31 (s, 1H), 7.23-7.16 (m, 2H), 5.28 (q, J=6.7 Hz, 1H), 5.15 (d, J=10.6 Hz, 1H), 4.58 (dd, J=10.7, 8.1 Hz, 1H), 2.82 (q, J=7.8 Hz, 1H), 2.20 (d, J=8.6 Hz, 3H), 1.68 (s, 3H), 1.57 (d, J=6.8 Hz, 3H), 0.91 (dq, J=7.7, 2.4 Hz, 3H) ppm. ESI-MS m/z calc. 497.15494, found 498.4 (M+1)⁺; 496.4 (M−1)⁻; Retention time: 3.14 minutes.

### Example 24

(2R,3S,4S,5R)-3-(2-(2-(1H-pyrazol-4-yl)ethyl)-3,4-difluorophenyl)-N-(1-(difluoromethyl)-3-methyl-1H-pyrazol-4-yl)-4,5-dimethyl-5-(trifluoromethyl)tetrahydrofuran-2-carboxamide (681)

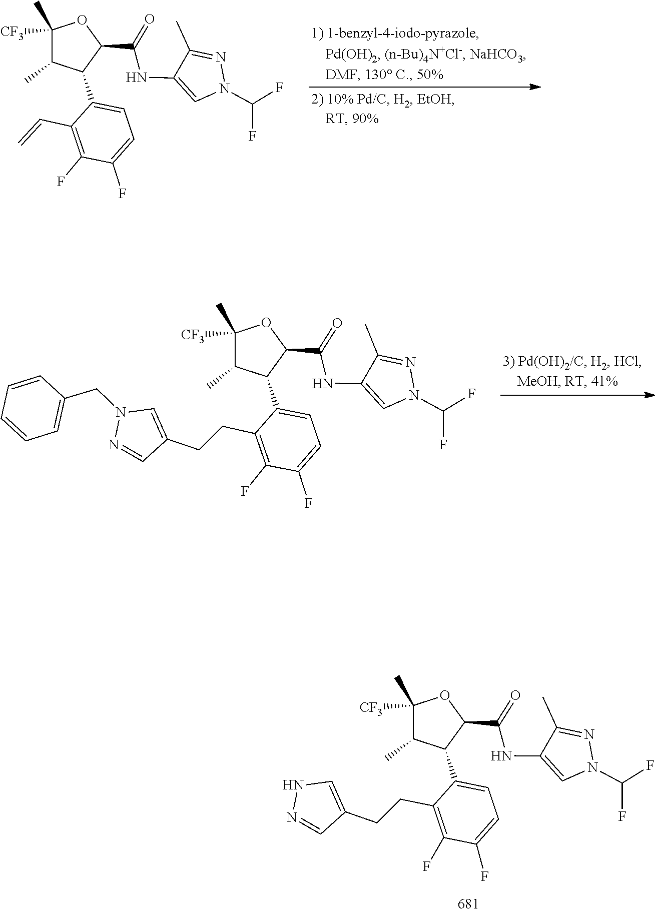 [SMILES: C=Cc1c([C@H]2[C@H](C(=O)Nc3cn(C(F)F)nc3C)O[C@@](C)(C(F)(F)F)[C@H]2C)ccc(F)c1F ; Cc1nn(C(F)F)cc1NC(=O)[C@@H]1O[C@@](C)(C(F)(F)F)[C@@H](C)[C@H]1c1ccc(F)c(F)c1CCc1cnn(Cc2ccccc2)c1 ; Cc1nn(C(F)F)cc1NC(=O)[C@@H]1O[C@@](C)(C(F)(F)F)[C@@H](C)[C@H]1c1ccc(F)c(F)c1CCc1cn[nH]c1]

To a solution of (2R,3S,4S,5R)—N-[1-(difluoromethyl)-3-methyl-pyrazol-4-yl]-3-(3,4-difluoro-2-vinyl-phenyl)-4,5-dimethyl-5-(trifluoromethyl)tetrahydrofuran-2-carboxamide (128 mg, 0.2670 mmol) in DMF (1 mL) was added 1-benzyl-4-iodo-pyrazole (83 mg, 0.29 mmol), diacetoxypalladium (3 mg, 0.013 mmol), NaHCO₃ (56 mg, 0.66 mmol), and tetrabutylammonium chloride (74 mg, 0.266 mmol). The reaction mixture was subject to microwave heating at 130° C. for 30 min. The reaction mixture was partitioned between EtOAc and water and the organic layer was separated. The aqueous layer was extracted with EtOAc and the combined organic extracts were dried (MgSO₄), filtered and concentrated in vacuo. Purification by silica gel chromatography (SiO₂, 0 to 100% EtOAc in hexane) gave (2R,3S,4S,5R)-3-[2-[(E)-2-(1-benzylpyrazol-4-yl)vinyl]-3,4-difluoro-phenyl]-N-[1-(difluoromethyl)-3-methyl-pyrazol-4-yl]-4,5-dimethyl-5-(trifluoromethyl)tetrahydrofuran-2-carboxamide (93 mg, 50%). ¹H NMR (500 MHz, Chloroform-d) δ 8.31 (s, 1H), 7.94 (s, 1H), 7.46-7.35 (m, 2H), 7.28-7.21 (m, 2H), 7.20-7.10 (m, 1H), 7.11-6.99 (m, 2H), 6.92-6.81 (m, 2H), 5.78 (d, J=9.7 Hz, 1H), 5.42 (d, J=10.0 Hz, 1H), 5.38-5.28 (m, 1H), 5.05 (d, J=5.1 Hz, 1H), 4.89 (d, J=9.0 Hz, 2H), 4.01 (s, 1H), 2.81 (t, J=7.8 Hz, 1H), 2.31 (d, J=18.1 Hz, 3H), 1.69 (s, 3H), 0.83 (d, J=7.4 Hz, 3H) ppm. ESI-MS m/z calc. 635.21313, found 636.4 (M+1)⁺; 634.4 (M−1)⁻; Retention time 1.08 minutes.

### Step 2:

To a solution of (2R,3S,4S,5R)-3-[2-[(E)-2-(1-benzylpyrazol-4-yl)vinyl]-3,4-difluoro-phenyl]-N-[1-(difluoromethyl)-3-methyl-pyrazol-4-yl]-4,5-dimethyl-5-(trifluoromethyl)tetrahydrofuran-2-carboxamide (90 mg, 0.1289 mmol) in EtOH (5 mL) that had been degassed with nitrogen was added palladium on carbon (5 mg of 10 wt. % loading, 0.046 mmol). The reaction mixture was stirred under a hydrogen atmosphere at ambient temperature for 16 h. The reaction mixture was filtered through a pad of celite, washing through with MeOH. The combined filtrates were concentrated in vacuo to give (2R,3S,4S,5R)-3-[2-[2-(1-benzylpyrazol-4-yl)ethyl]-3,4-difluoro-phenyl]-N-[1-(difluoromethyl)-3-methyl-pyrazol-4-yl]-4,5-dimethyl-5-(trifluoromethyl)tetrahydrofuran-2-carboxamide (74 mg, 90%). ESI-MS m/z calc. 637.22876, found 638.3 (M+1)⁺; 636.3 (M−1)⁻; Retention time: 1.07 minutes

### Step 3:

To a solution of (2R,3S,4S,5R)-3-[2-[2-(1-benzylpyrazol-4-yl)ethyl]-3,4-difluoro-phenyl]-N-[1-(difluoromethyl)-3-methyl-pyrazol-4-yl]-4,5-dimethyl-5-(trifluoromethyl)tetrahydrofuran-2-carboxamide (74 mg, 0.1161 mmol) in MeOH (7 mL) was added palladium hydroxide on carbon (16 mg of 20 wt. % loading, 50% wet support, 0.114 mol) and HCl (290 μL of 1 M aqueous solution, 0.29 mmol). The reaction mixture was stirred under a hydrogen atmosphere at ambient temperature for 16 h. The reaction mixture was filtered through a celite pad and concentrated in vacuo. Purification by reverse phase HPLC-MS using a X-bridge C18 column (150×19 mm, 5 m particle size) from Waters gave (2R,3S,4S,5R)-3-(2-(2-(1H-pyrazol-4-yl)ethyl)-3,4-difluorophenyl)-N-(1-(difluoromethyl)-3-methyl-1H-pyrazol-4-yl)-4,5-dimethyl-5-(trifluoromethyl)tetrahydrofuran-2-carboxamide (681, 26.4 mg, 41%). ¹H NMR (500 MHz, DMSO-d₆) δ 12.57 (s, 1H), 9.98 (s, 1H), 8.31 (s, 2H), 7.81-7.47 (m, 1H), 7.32 (q, J=8.9 Hz, 1H), 7.19 (dd, J=9.1, 4.7 Hz, 1H), 5.21 (d, J=10.5 Hz, 1H), 4.23 (dd, J=10.6, 7.5 Hz, 2H), 2.90 (dt, J=45.3, 11.1 Hz, 2H), 2.80-2.54 (m, 3H), 2.14 (s, 3H), 1.58 (s, 3H), 0.75 (d, J=7.4 Hz, 3H) ppm. ESI-MS m/z calc. 547.1818, found 548.3 (M+1)⁺; 546.3 (M−1)⁻; Retention time: 3.26 minutes.

### Example 25

### 5-((2R,3S,4S,5R)-3-(4-(difluoromethoxy)-3-fluoro-2-methoxyphenyl)-4,5-dimethyl-5-(trifluoromethyl)tetrahydrofuran-2-carboxamido)picolinamide (682)

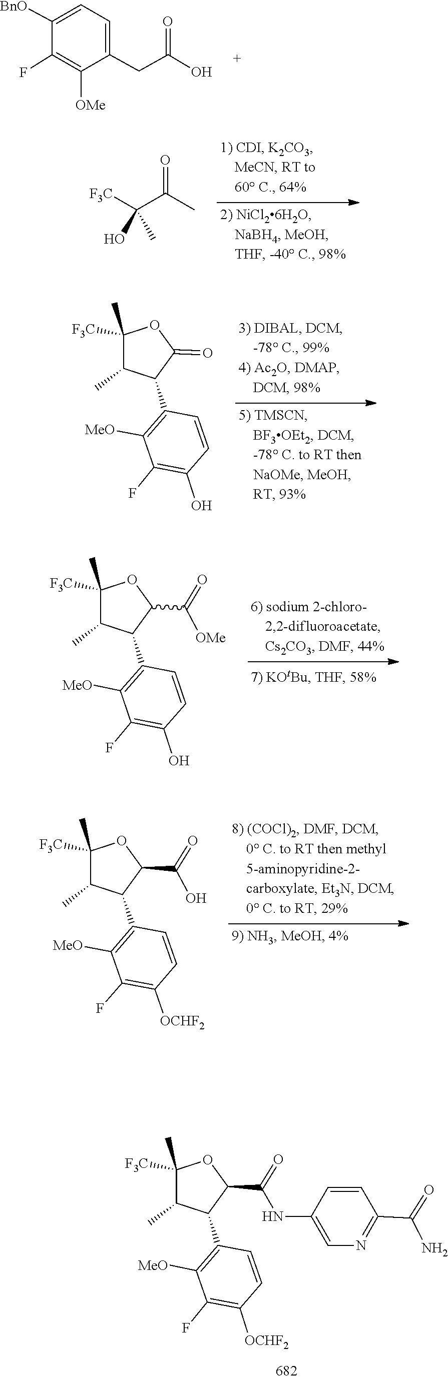

### Step 1:

To a solution of 2-(4-(benzyloxy)-3-fluoro-2-methoxyphenyl)acetic acid (9.8 g, 32.072 mmol) in MeCN (100 mL) was added CDI (6 g, 37.003 mmol) and the reaction mixture was heated at 40° C. for 15 min. (R)-4,4,4-trifluoro-3-hydroxy-3-methylbutan-2-one (6 g, 38.436 mmol) and potassium carbonate (5.5 g, 39.796 mmol) were added and the reaction mixture was heated at 60° C. for 30 h. The reaction mixture was diluted with water (50 mL) and extracted with MTBE (2×100 mL). The combined organic extracts were washed with HCl (2×50 mL of 2 M aqueous solution), dried (Na₂SO₄), filtered and concentrated in vacuo. Purification by flash chromatography (120 g SiO₂, 0 to 100% EtOAc in heptane) gave (R)-3-(4-(benzyloxy)-3-fluoro-2-methoxyphenyl)-4,5-dimethyl-5-(trifluoromethyl)furan-2(5H)-one (9.17 g, 64%) as a yellow solid. ¹H NMR (400 MHz, Chloroform-d) δ 7.46-7.34 (m, 5H), 6.91 (dd, J=8.7, 1.8 Hz, 1H), 6.80 (dd, J=8.7, 7.8 Hz, 1H), 5.16 (s, 2H), 3.85 (d, J=1.8 Hz, 3H), 2.03 (s, 3H), 1.73 (s, 3H) ppm. ESI-MS m/z calc. 410.1141, found 411.23 (M+1)⁺; Retention time: 2.97 minutes.

### Step 2:

Nickel dichloride hexahydrate (1.8 g, 7.573 mmol) was added to solution of (R)-3-(4-(benzyloxy)-3-fluoro-2-methoxyphenyl)-4,5-dimethyl-5-(trifluoromethyl)furan-2(5H)-one (3 g, 7.311 mmol) in MeOH (300 mL) and THF (60 mL) at −40° C. under a nitrogen atmosphere. NaBH₄ (1.4 g, 37.00 mmol) was added portion-wise and the reaction mixture was stirred −40° C. 6 further additions of NiCl₂·6H₂O (1.8 g, 7.573 mmol) and NaBH₄ (1.4 g, 37.00 mmol) were made over a 2 h period. NH₄Cl (100 mL of saturated aqueous solution) was added and the mixture extracted with DCM (100 mL). The organic extract was dried (MgSO₄), filtered and concentrated in vacuo to give (3S,4S,5R)-3-(3-fluoro-4-hydroxy-2-methoxyphenyl)-4,5-dimethyl-5-(trifluoromethyl)dihydrofuran-2(3H)-one (2.3 g, 98%). ESI-MS m/z calc. 322.08282, found 321.4 (M−1)⁻; Retention time: 0.79 minutes.

### Step 3:

To a stirred solution of (3S,4S,5R)-3-(3-fluoro-4-hydroxy-2-methoxyphenyl)-4,5-dimethyl-5-(trifluoromethyl)dihydrofuran-2(3H)-one (2.3 g, 7.137 mmol) in DCM (40 mL) at −78° C. under a nitrogen atmosphere was added DIBAL (15 mL of 1 M in DCM, 15 mmol) dropwise. The reaction mixture was stirred at −78° C. The reaction mixture was quenched by addition of NH₄Cl (100 mL of saturated aqueous solution) and Rochelle's salt (100 mL of 30% w/w aqueous solution). The resulting mixture was vigorously stirred at ambient temperature until a clear phase separation was achieved. The organic layer was dried (MgSO₄), filtered and concentrated in vacuo to give (3S,4S,5R)-3-(3-fluoro-4-hydroxy-2-methoxyphenyl)-4,5-dimethyl-5-(trifluoromethyl)tetrahydrofuran-2-ol (2.3 g, 99%). ESI-MS m/z calc. 324.09848, found 323.4 (M−1)⁻; Retention time: 0.73 minutes.

### Step 4:

To a solution of (3S,4S,5R)-3-(3-fluoro-4-hydroxy-2-methoxyphenyl)-4,5-dimethyl-5-(trifluoromethyl)tetrahydrofuran-2-ol (380 mg, 1.172 mmol) in DCM (4 mL) was added DMAP (210 mg, 1.719 mmol) and acetic anhydride (700 μL, 7.419 mmol). The reaction mixture was stirred at ambient temperature. After 2 hours, the mixture was quenched by addition of NaHCO₃ (30 mL of saturated aqueous solution). The mixture was diluted with DCM (20 mL) and extracted with DCM (10 mL). The combined organic extracts were dried (MgSO₄), filtered and concentrated in vacuo. Purification by flash chromatography (24 g SiO₂, 0 to 100% EtOAc in heptane) gave (3S,4S,5R)-3-(4-acetoxy-3-fluoro-2-methoxyphenyl)-4,5-dimethyl-5-(trifluoromethyl)tetrahydrofuran-2-yl acetate (470 mg, 98%) as a mixture of epimers at the C₂ position. ESI-MS m/z calc. 408.1196, found 407.3 (M−1)⁻; Retention time: 1.01 minutes.

### Step 5:

TMSCN (400 μL, 3.000 mmol) and BF₃·OEt₂ (1000 μL, 8.103 mmol) were added successively to a solution of (3S,4S,5R)-3-(4-acetoxy-3-fluoro-2-methoxyphenyl)-4,5-dimethyl-5-(trifluoromethyl)tetrahydrofuran-2-yl acetate (470 mg, 1.151 mmol) in DCM (15 mL) at −78° C. The reaction mixture was stirred at −78° C. for 30 min and then allowed to warm to ambient temperature. After 30 minutes, the mixture was quenched with NaHCO₃ (60 mL of saturated aqueous solution). The mixture was extracted with DCM (3×30 mL). The combined organic extracts were dried (Na₂SO₄), filtered and concentrated in vacuo to give 4-((3S,4S,5R)-2-cyano-4,5-dimethyl-5-(trifluoromethyl)tetrahydrofuran-3-yl)-2-fluoro-3-methoxyphenyl acetate (400 mg, 93%). ESI-MS m/z calc. 375.10938, found 374.5 (M−1)⁻; Retention time: 1.0 minutes. To a solution of 4-((3S,4S,5R)-2-Cyano-4,5-dimethyl-5-(trifluoromethyl)tetrahydrofuran-3-yl)-2-fluoro-3-methoxyphenyl acetate in MeOH (7 mL) was added NaOMe solution (800 μL of 25% w/w in MeOH, 3.498 mmol) and the reaction mixture was stirred at ambient temperature overnight. A saturated citric acid solution was added and the reaction mixture was stirred at ambient temperature for 4 h. The mixture was extracted with DCM (2×30 mL). The combined organic extracts were dried (MgSO₄), filtered and concentrated in vacuo to give methyl (2R,3S,4S,5R)-3-(3-fluoro-4-hydroxy-2-methoxyphenyl)-4,5-dimethyl-5-(trifluoromethyl)tetrahydrofuran-2-carboxylate (380 mg, 90%). ESI-MS m/z calc. 366.10904, found 365.4 (M−1)⁻; Retention time: 0.87 minutes.

### Step 6:

Sodium 2-chloro-2,2-difluoroacetate (1.1 g, 7.168 mmol) was added to a mixture of methyl (2R,3S,4S,5R)-3-(3-fluoro-4-hydroxy-2-methoxyphenyl)-4,5-dimethyl-5-(trifluoromethyl)tetrahydrofuran-2-carboxylate (1.01 g, 2.757 mmol) and Cs₂CO₃ (2.7 g, 8.287 mmol) in DMF (10 mL). The reaction mixture was heated to 90° C. Upon reaction completion, the mixture was partitioned between DCM (20 mL) and water (50 mL). The organic extract was dried (MgSO₄), filtered, and concentrated in vacuo. Purification by flash chromatography (12 g SiO₂, 0 to 100% EtOAc in heptane) gave methyl (2R,3S,4S,5R)-3-(4-(difluoromethoxy)-3-fluoro-2-methoxyphenyl)-4,5-dimethyl-5-(trifluoromethyl)tetrahydrofuran-2-carboxylate (500 mg, 44%). ESI-MS m/z calc. 416.10583, Retention time: 0.87 minutes; no mass ionisation.

### Step 7:

To a solution of methyl (2R,3S,4S,5R)-3-(4-(difluoromethoxy)-3-fluoro-2-methoxyphenyl)-4,5-dimethyl-5-(trifluoromethyl)tetrahydrofuran-2-carboxylate (180 mg, 0.4324 mmol) in THF (3 mL) at ambient temperature was added KO-t-Bu (200 mg, 1.782 mmol). The reaction mixture was stirred at ambient temperature for 5 min. The reaction mixture was quenched by addition of NH₄Cl (3 mL of saturated aqueous solution) and diluted with DCM (3 mL). The organic layer was separated and the aqueous phase was washed with DCM (5 mL). The combined organic extracts were dried (MgSO₄), filtered and concentrated in vacuo to give (2R,3S,4S,5R)-3-(4-(difluoromethoxy)-3-fluoro-2-methoxyphenyl)-4,5-dimethyl-5-(trifluoromethyl)tetrahydrofuran-2-carboxylic acid (100 mg, 58%), which was used in the next step without further purification. ESI-MS m/z calc. 402.09018, found 401.4 (M−1)⁻; Retention time: 0.6 minutes.

### Step 8:

Oxalyl chloride (60 μL, 0.287 mmol) was added to a solution of (2R,3S,4S,5R)-3-(4-(difluoromethoxy)-3-fluoro-2-methoxyphenyl)-4,5-dimethyl-5-(trifluoromethyl)tetrahydrofuran-2-carboxylic acid (120 mg, 0.298 mmol) and DMF (3 μL, 0.026 mmol) in DCM (1.2 mL). The reaction mixture was stirred at ambient temperature for 30 min. The reaction mixture was concentrated in vacuo then dissolved in DCM (1 mL) and treated with Et₃N (60 μL, 0.179 mmol) and methyl 5-aminopyridine-2-carboxylate. The mixture was stirred ambient temperature for 1 h and then quenched by the addition of MeOH (100 μL). The mixture was concentrated in vacuo. Purification by flash chromatography (4 g SiO₂, 0 to 35% EtOAc in heptane) gave methyl 5-[[(2R,3S,4S,5R)-3-[4-(difluoromethoxy)-3-fluoro-2-methoxy-phenyl]-4,5-dimethyl-5-(trifluoromethyl)tetrahydrofuran-2-carbonyl]amino]pyridine-2-carboxylate (47 mg, 29%) ESI-MS m/z calc. 536.1382, Retention time: 0.97 minutes (no MS ionisation)

### Step 9:

A solution of methyl 5-[[(2R,3S,4S,5R)-3-[4-(difluoromethoxy)-3-fluoro-2-methoxy-phenyl]-4,5-dimethyl-5-(trifluoromethyl)tetrahydrofuran-2-carbonyl]amino]pyridine-2-carboxylate in NH₃ (10 mL of 7 M, 70 mmol) was stirred at ambient temperature for 4 h. The mixture was concentrated in vacuo. Purification by flash chromatography (4 g SiO₂, 0 to 100% EtOAc in heptane). Final purification was by chiral SFC separation. SFC Amylose: Column: Chiralpak AS-H, 10×250 mm. Mobile phase: 35% MeOH (containing 20 mM Ammonia), 65% CO2. Flow: 10 mL/min gave 5-((2R,3S,4S,5R)-3-(4-(difluoromethoxy)-3-fluoro-2-methoxyphenyl)-4,5-dimethyl-5-(trifluoromethyl)tetrahydrofuran-2-carboxamido)picolinamide (682, 6.9 mg, 4%). ¹H NMR (500 MHz, DMSO-d₆) δ 10.65 (s, 1H), 8.84 (dd, J=2.5, 0.7 Hz, 1H), 8.21 (dd, J=8.6, 2.5 Hz, 1H), 8.03-7.97 (m, 2H), 7.54-7.50 (m, 1H), 7.25 (t, J=73.2 Hz, 1H), 7.19 (dd, J=8.8, 1.8 Hz, 1H), 7.10 (t, J=8.1 Hz, 1H), 5.14 (d, J=10.2 Hz, 1H), 4.29 (dd, J=10.3, 7.7 Hz, 1H), 3.94 (d, J=1.9 Hz, 3H), 2.80 (p, J=7.5 Hz, 1H), 1.62 (s, 3H), 0.77-0.72 (m, 3H). ESI-MS m/z calc. 521.13855, found 522.1 (M+1)⁺; 520.1 (M−1)⁻; Retention time: 3.28 minutes as a white solid.

### Example 26

rel-(2S,3R,5S)-3-(3,4-difluoro-2-methoxyphenyl)-5-methyl-N-(3-methyl-1-(methylsulfonyl)-1H-pyrazol-4-yl)-5-(trifluoromethyl)tetrahydrofuran-2-carboxamide (683) and rel-(2R,3S,5R)-3-(3,4-difluoro-2-methoxyphenyl)-5-methyl-N-(3-methyl-1-(methylsulfonyl)-1H-pyrazol-4-yl)-5-(trifluoromethyl)tetrahydrofuran-2-carboxamide (684)

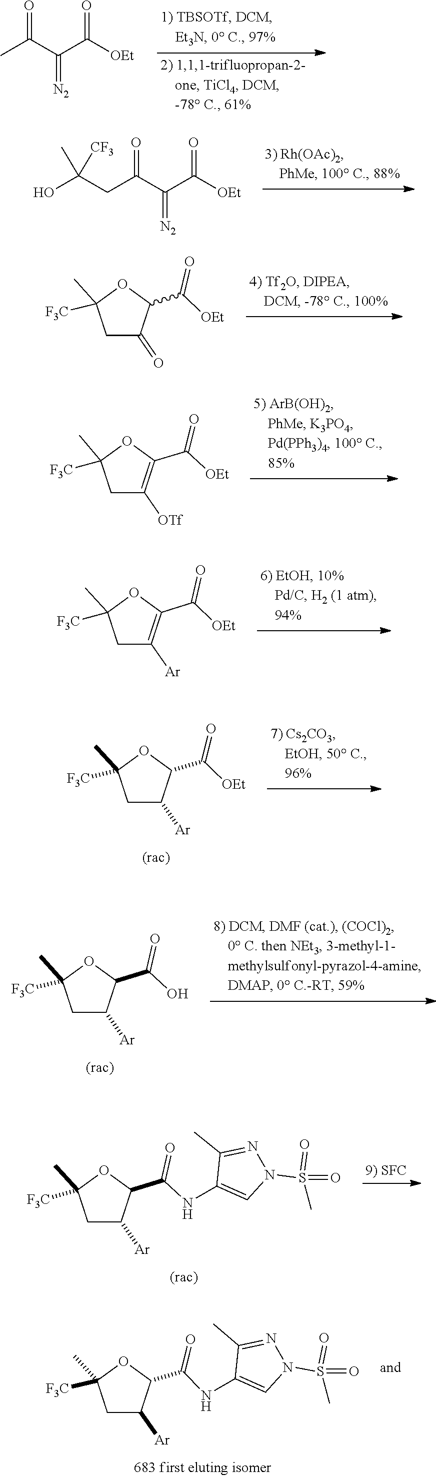

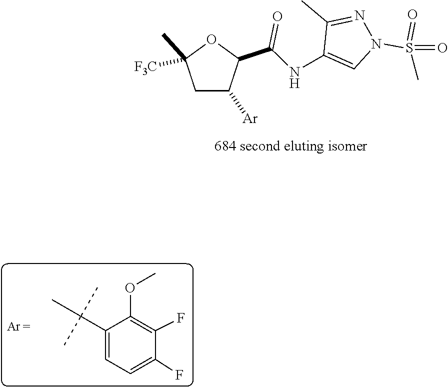

### Step 1:

Triethylamine (8.05 g, 11.2 mL, 78.8 mmol) was added to a stirred solution of ethyl 2-diazo-3-oxobutanoate (5.0 g, 31.4 mmol) in DCM (50 mL) at 0° C. TBSOTf (9.24 g, 8.2 mL, 34.3 mmol) was added slowly and the reaction mixture was stirred for 30 min at 0° C. The reaction mixture was washed with a 30% NaHCO₃ solution (200 mL). The organic layer was separated, washed with water (500 mL), dried (MgSO₄), filtered, and concentrated in vacuo to give ethyl 3-((tert-butyldimethylsilyl)oxy)-2-diazobut-3-enoate (8.22 g, 97%), which was used in the next step without further purification.

### Step 2:

A solution of 1,1,1-trifluoropropan-2-one (33.8 g, 27 mL, 301.2 mmol) in DCM (150 mL) was cooled down to −78° C. TiCl₄ (56.8 g, 33 mL, 299.2 mmol) was added dropwise to the stirred reaction mixture. The reaction was kept at −78° C. for 10 min before a solution of ethyl 3-((tert-butyldimethylsilyl)oxy)-2-diazobut-3-enoate (64 g, 236.7 mmol) in DCM (150 mL) was added dropwise. The reaction was kept at −78° C. for 1 h. A saturated solution of NaHCO₃ was added and the mixture was diluted with DCM. The organic layer was separated, dried (MgSO₄), filtered and concentrated in vacuo. Purification by silica gel chromatography (SiO₂, 0 to 30% EtOAc in hexane) gave ethyl 2-diazo-6,6,6-trifluoro-5-hydroxy-5-methyl-3-oxohexanoate (39 g, 61%) as a pale yellow liquid. ¹H NMR (400 MHz, Chloroform-d) δ 4.92 (s, 1H), 4.32 (q, J=7.1 Hz, 2H), 3.63 (d, J=15.5 Hz, 1H), 2.84 (d, J=15.5 Hz, 1H), 1.41 (s, 3H), 1.33 (t, J=7.1 Hz, 3H) ppm.

### Step 3:

Rhodium (II) acetate (643 mg, 1.45 mmol) was charged into an oven dried two necked flask. Toluene (970 mL) was added and the solution was stirred at 100° C. for 10 min. The solution was briefly lifted out of the oil bath whilst a solution of ethyl 2-diazo-6,6,6-trifluoro-5-hydroxy-5-methyl-3-oxohexanoate (39 g, 145.4 mmol) in a toluene (200 mL) was added dropwise. The reaction mixture was heated at reflux for 1 h. The mixture was filtered through filter paper and the filtrate was concentrated in vacuo to give ethyl 5-methyl-3-oxo-5-(trifluoromethyl)tetrahydrofuran-2-carboxylate (30.89 g, 88%) as a mixture of epimers at the position next to the ester. ¹H NMR (400 MHz, Chloroform-d) δ 4.68 (s, 1H), 4.35-4.17 (m, 2H), 2.89 (d, J=18.8, 1H), 2.58 (d, J=18.8, 1H), 1.70 (s, 3H), 1.30 (t, J=7.2, Hz, 3H) ppm.

### Step 4:

Trifluoromethanesulfonic anhydride (6.0 mL, 35.7 mmol) was added dropwise to a solution of ethyl 5-methyl-3-oxo-5-(trifluoromethyl)tetrahydrofuran-2-carboxylate (6.5 g, 27.1 mmol) and DIPEA (14 mL, 80.4 mmol) in DCM (150 mL) at −78° C. The reaction mixture was stirred for 2.5 h before saturated aqueous NH₄Cl (75 mL) was added. The mixture was warmed up to ambient temperature. The aqueous layer was extracted with DCM (2×30 mL). The combined organic extracts were dried (MgSO₄), filtered and concentrated in vacuo to give ethyl 5-methyl-5-(trifluoromethyl)-3-(((trifluoromethyl)sulfonyl)oxy)-4,5-dihydrofuran-2-carboxylate (10.1 g), which was used directly in the next reaction.

### Step 5:

K₃PO₄ (13 mL of 2 M aq., 26.0 mmol) was added to a stirred solution of (3,4-difluoro-2-methoxyphenyl)boronic acid (2.0 g, 10.6 mmol) and ethyl 5-methyl-5-(trifluoromethyl)-3-(((trifluoromethyl)sulfonyl)oxy)-4,5-dihydrofuran-2-carboxylate (3 g, 7.90 mmol) in toluene (80 mL). The mixture was degassed by bubbling nitrogen through the solution for 20 min. Pd(PPh₃)₄ (466 mg, 0.40 mmol) was added and the reaction was heated to 100° C. for 1 h. The mixture was filtered through a pad of celite. The filtrates were diluted with water (50 mL) and the phases were separated. The aqueous layer was extracted with EtOAc (50×2 mL). The combined organic extracts were dried (MgSO₄), filtered and concentrated in vacuo. Purification by silica gel chromatography (SiO₂, 0 to 2% EtOAc in hexane) gave ethyl 3-(3,4-difluoro-2-methoxyphenyl)-5-methyl-5-(trifluoromethyl)-4,5-dihydrofuran-2-carboxylate (2.5 g, 85%) as a light-yellow liquid. ¹H NMR (400 MHz, Chloroform-d) δ 6.87 (pd, J=8.8, 6.2 Hz, 2H), 4.15 (q, J=7.1 Hz, 2H), 3.89 (s, 3H), 3.42 (d, J=17.4 Hz, 1H), 2.93 (d, J=17.4 Hz, 1H), 1.65 (s, 3H), 1.14 (t, J=7.1 Hz, 3H) ppm. ESI-MS m/z calc. 366.08905, found 367.4 (M+1)⁺; Retention time: 1.01 minutes.

### Step 6:

EtOH (200 mL) was added to a mixture of ethyl 3-(3,4-difluoro-2-methoxyphenyl)-5-methyl-5-(trifluoromethyl)-4,5-dihydrofuran-2-carboxylate (5.51 g, 15.0 mmol) and Pd/C (10 wt. % loading, 2.2 g, 2.067 mmol). The mixture was degassed and stirred under a balloon of H₂ for 96 h. The catalyst was removed by filtration and the solids washed with EtOH (50 mL). The filtrates were concentrated in vacuo. A further portion of Pd/C (10 wt. % loading, 2.2 g, 2.07 mmol) was added to the residue followed by EtOH (200 mL). The reaction mixture was stirred under a balloon of H₂ at ambient temperature for 24 h. The catalyst was removed by filtration and the solids washed with EtOH (50 mL). The filtrates were concentrated in vacuo. A further portion of Pd/C (10 wt. % loading, 2.2 g, 2.07 mmol) was added to the residue followed by EtOH (200 mL) and the reaction mixture stirred under a balloon of H₂ at ambient temperature for 4 days. The catalyst was removed by filtration and the solids washed with EtOH (50 mL). The filtrates were concentrated in vacuo to give ethyl rac-(2S,3S,5R)-3-(3,4-difluoro-2-methoxyphenyl)-5-methyl-5-(trifluoromethyl)tetrahydrofuran-2-carboxylate (5.19 g, 94%) as a white solid, and as a single diastereomer. ¹H NMR (500 MHz, Chloroform-d) δ 6.89-6.86 (m, 1H), 6.82-6.77 (m, 1H), 4.93 (d, J=8.9 Hz, 1H), 4.23 (dt, J=13.0, 7.6 Hz, 1H), 4.08 (d, J=2.9 Hz, 3H), 3.85-3.71 (m, 2H), 2.82 (t, J=12.5 Hz, 1H), 2.04 (dd, J=12.0, 6.7 Hz, 1H), 1.53 (s, 3H), 0.94 (t, J=7.1 Hz, 3H) ppm; ¹⁹F NMR (471 MHz, Chloroform-d) δ−80.15, −136.84 (d, J=19.4 Hz), −154.77 (d, J=19.6 Hz) ppm.

### Step 7:

Ethyl rac-(2S,3S,5R)-3-(3,4-difluoro-2-methoxyphenyl)-5-methyl-5-(trifluoromethyl)tetrahydrofuran-2-carboxylate (5.19 g, 14.09 mmol) was dissolved in ethanol (100 mL). Cs₂CO₃ (7.1 g, 21.79 mmol) was added and the suspension stirred at 50° C. for 2 h. The reaction mixture was concentrated in vacuo and the residue partitioned between 1M HCl and MTBE. The aqueous layer was extracted twice with MTBE. The combined organic extracts were dried (MgSO₄), filtered and concentrated in vacuo to give rac-(2R,3S,5R)-3-(3,4-difluoro-2-methoxyphenyl)-5-methyl-5-(trifluoromethyl)tetrahydrofuran-2-carboxylic acid (5.1063 g, 96%) as a colourless oil. ¹H NMR (500 MHz, Chloroform-d) δ 6.99-6.96 (m, 1H), 6.92-6.87 (m, 1H), 4.68 (d, J=10.5 Hz, 1H), 4.00 (d, J=2.7 Hz, 3H), 3.90 (ddd, J=12.0, 10.6, 8.2 Hz, 1H), 2.58 (t, J=12.5 Hz, 1H), 2.31 (dd, J=13.0, 8.2 Hz, 1H), 1.60 (s, 3H) ppm; ¹⁹F NMR (471 MHz, Chloroform-d) δ−81.56, −136.40 (d, J=19.6 Hz), −153.60 (d, J=19.5 Hz) ppm. ESI-MS m/z calc. 340.0734, found 339.5 (M−1)⁻; Retention time: 0.52 minutes.

### Step 8:

To a solution of rac-(2R,3S,5R)-3-(3,4-difluoro-2-methoxyphenyl)-5-methyl-5-(trifluoromethyl)tetrahydrofuran-2-carboxylic acid (120 mg, 0.3174 mmol) in DCM (2 mL) was added DMF (5 μL, 0.06457 mmol) and carefully oxalyl chloride (100 μL, 1.146 mmol) at 0° C. Visible gas evolution. After 30 mins, the reaction mixture was concentrated in vacuo then diluted in DCM (2.5 mL) and added dropwise to a solution of 3-methyl-1-methylsulfonyl-pyrazol-4-amine (70 mg, 0.3995 mmol), DMAP (4 mg, 0.03274 mmol) and Et₃N (250 μL, 1.794 mmol) in DCM (2 mL) at 0° C. The reaction was warmed to ambient temperature after 10 minutes and stirred for 16 hours before being concentrated in vacuo. Purification by flash column chromatography (40 g SiO₂, 0 to 100% ethylacetate in heptane) gave rac-(2R,3S,5R)-3-(3,4-difluoro-2-methoxyphenyl)-5-methyl-N-(3-methyl-1-(methylsulfonyl)-1H-pyrazol-4-yl)-5-(trifluoromethyl)tetrahydrofuran-2-carboxamide (105 mg, 59%). ESI-MS m/z calc. 497.10437, found 496.0 (M−1)⁻; Retention time: 0.94 minutes.

### Step 9:

The enantiomers of rac-(2R,3S,5R)-3-(3,4-difluoro-2-methoxyphenyl)-5-methyl-N-(3-methyl-1-(methylsulfonyl)-1H-pyrazol-4-yl)-5-(trifluoromethyl)tetrahydrofuran-2-carboxamide (100 mg, 0.1890 mmol) were separated by chiral SFC.

First eluting isomer (rt=2.44 minutes): rel-(2S,3R,5S)-3-(3,4-difluoro-2-methoxyphenyl)-5-methyl-N-(3-methyl-1-(methylsulfonyl)-1H-pyrazol-4-yl)-5-(trifluoromethyl)tetrahydrofuran-2-carboxamide (683, 29.1 mg, 61%) ESI-MS m/z calc. 497.10437, found 498.1 (M+1); 496.1 (M−1); Retention time: 3.27 minutes.

Second eluting isomer (rt=3.50 minutes): rel-(2R,3S,5R)-3-(3,4-difluoro-2-methoxyphenyl)-5-methyl-N-(3-methyl-1-(methylsulfonyl)-1H-pyrazol-4-yl)-5-(trifluoromethyl)tetrahydrofuran-2-carboxamide (684, 32.4 mg, 697). ¹H NMR (500 MHz, DMSO-d₆) δ 9.84 (s, 1H), 8.33 (s, 1H), 7.25-7.10 (m, 2H), 4.76 (d, J=10.1 Hz, 1H), 4.05 (q, 4=10.0 Hz, 1H), 3.86 (d, (=1.9 Hz, 3H), 3.44 (s, 3H), 2.47-2.42 (i, 2H), 2.18 (s, 3H), 1.53 (s, 3H) ppm. ESI-MS m/z calc. 497.10437, found 498.1 (M+1); 496.1 (M−1); Retention time: 3.28 minutes.

The following compounds were made using a similar method to that described in Example 26, except that different coupling partners were used in the amide coupling step 8 and the separated isomers generated in step 9 were deprotected using General Method B.

| | | | | MS | |
|---|---|---|---|---|---|
| Cmpd | | LC/MS | Found | retention | |
| No. | Compound Name | (m/z calc.) | M + 1 | time | NMR (shifts in ppm) |
| 685 | rel-(2R,3S,5R)-3-(3,4- | 476.394 | 477.2 | 2.88 | ¹H NMR (500 MHz, |
| | difluoro-2- | | | | DMSO-d₆) δ 10.29 (s, |
| | methoxyphenyl)-N-(2- | | | | 1H), 8.34 (d, J = 5.5 Hz, |
| | (1,2- | | | | 1H), 7.69 (d, J = 2.0 Hz, |
| | dihydroxyethyl)pyridin- | | | | 1H), 7.55-7.54 (m, 1H), |
| | 4-yl)-5-methyl-5- | | | | 7.19-7.15 (m, 2H), 5.38 |
| | (trifluoromethyl)tetrahydrofuran-2- | | | | (d, J = 4.7 Hz, 1H), 4.66 |
| | carboxamide | | | | (d, J = 9.8 Hz, 2H), 4.55- |
| | (derived from | | | | 4.51 (m, 1H), 4.06 (q, J = |
| | Intermediate P, first | | | | 10.1 Hz, 1H), 3.87 (d, J = |
| | eluting isomer; first | | | | 2.0 Hz, 3H), 3.74- |
| | eluting isomer in SFC | | | | 3.55 (m, 1H), 3.44 (dt, J = |
| | separation (Step 9)) | | | | 11.6, 6.4 Hz, 1H), 1.56 |
| | | | | | (s, 3H) ppm. |
| 686 | rel-(2S,3R,5S)-3-(3,4- | 476.394 | 476.8 | 2.88 | ¹H NMR (500 MHz, |
| | difluoro-2- | | | | DMSO-d₆) δ 10.31 (s, |
| | methoxyphenyl)-N-(2- | | | | 1H), 8.34 (d, J = 5.6 Hz, |
| | (1,2- | | | | 1H), 7.70 (d, J = 2.0 Hz, |
| | dihydroxyethyl)pyridin- | | | | 1H), 7.54 (dd, J = 5.8, |
| | 4-yl)-5-methyl-5- | | | | 2.0 Hz, 1H), 7.19-7.15 |
| | (trifluoromethyl)tetrahydrofuran-2- | | | | (m, 2H), 5.39 (s, 1H), |
| | carboxamide | | | | 4.66 (d, J = 10.0 Hz, 2H), |
| | (derived from | | | | 4.55-4.52 (m, 1H), 4.06 |
| | Intermediate P, first | | | | (q, J = 10.1 Hz, 1H), 3.87 |
| | eluting isomer; second | | | | (d, J = 2.0 Hz, 3H), 3.65 |
| | eluting isomer in SFC | | | | (dd, J = 10.7, 5.1 Hz, |
| | separation (Step 9)) | | | | 1H), 3.44 (dt, J = 11.5, |
| | | | | | 6.0 Hz, 1H), 1.56 (s, 3H) |
| | | | | | ppm. 2 × H hidden by |
| | | | | | DMSO. |
| 687 | rel-(2R,3S,5R)-3-(3,4- | 476.394 | 477 | 2.88 | ¹H NMR (500 MHz, |
| | difluoro-2- | | | | DMSO-d₆) δ 10.31 (s, |
| | methoxyphenyl)-N-(2- | | | | 1H), 8.34 (d, J = 5.6 Hz, |
| | (1,2- | | | | 1H), 7.70 (d, J = 2.0 Hz, |
| | dihydroxyethyl)pyridin- | | | | 1H), 7.54 (d, J = 5.5 Hz, |
| | 4-yl)-5-methyl-5- | | | | 1H), 7.19-7.17 (dd, J = |
| | (trifluoromethyl)tetrahydrofuran-2- | | | | 8.5, 4.4 Hz, 2H), 5.38 (s, |
| | carboxamide | | | | 1H), 4.66 (d, J = 10.0 Hz, |
| | (derived from | | | | 2H), 4.55-4.52 (s, 1H), |
| | Intermediate P, second | | | | 4.06 (q, J = 9.9 Hz, 1H), |
| | eluting isomer; first | | | | 3.87 (d, J = 2.0 Hz, 3H), |
| | eluting isomer in SFC | | | | 3.67-3.63 (m, 1H), 3.46- |
| | separation (Step 9)) | | | | 3.43 (m, 1H), 1.56 (s, |
| | | | | | 3H) ppm. 2 × H hidden |
| | | | | | by DMSO. |
| 688 | rel-(2S,3R,5S)-3-(3,4- | 476.394 | 476.9 | 2.88 | ¹H NMR (500 MHz, |
| | difluoro-2- | | | | DMSO-d₆) δ 10.29 (s, |
| | methoxyphenyl)-N-(2- | | | | 1H), 8.34 (d, J = 5.5 Hz, |
| | (1,2- | | | | 1H), 7.69 (d, J = 2.0 Hz, |
| | dihydroxyethyl)pyridin- | | | | 1H), 7.54 (dd, J = 5.6, |
| | 4-yl)-5-methyl-5- | | | | 2.1 Hz, 1H), 7.19-7.15 |
| | (trifluoromethyl)tetrahydrofuran-2- | | | | (m, 2H), 5.37 (d, J = 4.7 |
| | carboxamide | | | | Hz, 1H), 4.67-4.65 (m, |
| | (derived from | | | | 2H), 4.54-4.51 (m, 1H), |
| | Intermediate P, second | | | | 4.06 (q, J = 9.9 Hz, 1H), |
| | eluting isomer; second | | | | 3.87 (d, J = 2.0 Hz, 3H), |
| | eluting isomer in SFC | | | | 3.66 (ddd, J = 10.4, 6.1, |
| | separation (Step 9)) | | | | 4.0 Hz, 1H), 3.44 (dt, J = |
| | | | | | 11.7, 6.4 Hz, 1H), 1.56 |
| | | | | | (s, 3H) ppm. 2 x H |
| | | | | | hidden by DMSO. |
| 689 | rel-(2R,3S,5R)-3-(3,4- | 494.384 | 494.8 | 3.02 | ¹H NMR (500 MHz, |
| | difluoro-2- | | | | DMSO-d₆) δ 9.97 (s, |
| | methoxyphenyl)-N-(2- | | | | 1H), 8.44 (d, J = 2.3 Hz, |
| | (1,2-dihydroxyethyl)-5- | | | | 1H), 8.15 (d, J = 6.5 Hz, |
| | fluoropyridin-4-yl)-5- | | | | 1H), 7.22-7.16 (dd, J = |
| | methyl-5- | | | | 9.4, 6.2 Hz, 2H), 5.45 (d, |
| | (trifluoromethyl)tetrahydrofuran-2- | | | | J = 4.8 Hz, 1H), 4.86 (d, |
| | carboxamide | | | | J = 10.2 Hz, 1H), 4.65 (t, |
| | (derived from | | | | J = 5.9 Hz, 1H), 4.55- |
| | Intermediate M, first | | | | 4.52 (m, 1H), 4.06 (q, J = |
| | eluting isomer; first | | | | 10.0 Hz, 1H), 3.88 (d, J = |
| | eluting isomer in SFC | | | | 2.0 Hz, 3H), 3.63 (ddd, J = |
| | separation (Step 9)) | | | | 10.5, 6.1, 4.1 Hz, 1H), |
| | | | | | 3.44 (dt, J = 11.1, 6.4 Hz, |
| | | | | | 1H), 1.56 (s, 3H) ppm. 2 × |
| | | | | | H hidden by DMSO. |
| 690 | rel-(2S,3R,5S)-3-(3,4- | 494.384 | 494.8 | 3.02 | ¹H NMR (500 MHz, |
| | difluoro-2- | | | | DMSO-d₆) δ 9.97 (s, |
| | methoxyphenyl)-N-(2- | | | | 1H), 8.44 (d, J = 2.3 Hz, |
| | (1,2-dihydroxyethyl)-5- | | | | 1H), 8.16 (d, J = 6.6 Hz, |
| | fluoropyridin-4-yl)-5- | | | | 1H), 7.23-7.16 (m, 2H), |
| | methyl-5- | | | | 5.45 (d, J = 4.8 Hz, 1H), |
| | (trifluoromethyl)tetrahydrofuran-2- | | | | 4.85 (d, J = 10.1 Hz, 1H), |
| | carboxamide | | | | 4.66 (t, J = 5.9 Hz, 1H), |
| | (derived from | | | | 4.54 (q, J = 4.8 Hz, 1H), |
| | Intermediate M, first | | | | 4.06 (q, J = 10.0 Hz, 1H), |
| | eluting isomer; second | | | | 3.88 (d, J = 2.1 Hz, 3H), |
| | eluting isomer in SFC | | | | 3.64 (ddd, J = 10.4, 6.0, |
| | separation (Step 9)) | | | | 4.0 Hz, 1H), 3.45 (dt, J = |
| | | | | | 10.9, 6.3 Hz, 1H), 1.57 |
| | | | | | (s, 3H) ppm. 2 x H |
| | | | | | hidden by DMSO. |
| 691 | rel-(2R,3S,5R)-3-(3,4- | 494.384 | 494.9 | 3.02 | ¹H NMR (500 MHz, |
| | difluoro-2- | | | | DMSO-d₆) δ 9.97 (s, |
| | methoxyphenyl)-N-(2- | | | | 1H), 8.44 (d, J = 2.3 Hz, |
| | (1,2-dihydroxyethyl)-5- | | | | 1H), 8.16 (d, J = 6.5 Hz, |
| | fluoropyridin-4-yl)-5- | | | | 1H), 7.21-7.16 (m, 2H), |
| | methyl-5- | | | | 5.45 (d, J = 4.8 Hz, 1H), |
| | (trifluoromethyl)tetrahydrofuran-2- | | | | 4.85 (d, J = 10.1 Hz, 1H), |
| | carboxamide | | | | 4.66 (t, J = 6.0 Hz, 1H), |
| | (derived from | | | | 4.54 (q, J = 4.9 Hz, 1H), |
| | Intermediate M, second | | | | 4.06 (q, J = 10.1 Hz, 1H), |
| | eluting isomer; first | | | | 3.88 (d, J = 2.0 Hz, 3H), |
| | eluting isomer in SFC | | | | 3.66-3.62 (m, 1H), 3.47- |
| | separation (Step 9)) | | | | 3.42 (m, 1H), 1.57 (s, |
| | | | | | 3H) ppm. 2 × H hidden |
| | | | | | by DMSO. |
| 692 | rel-(2S,3R,5S)-3-(3,4- | 494.384 | 494.9 | 3.02 | ¹H NMR (500 MHz, |
| | difluoro-2- | | | | DMSO-d₆) δ 9.97 (s, |
| | methoxyphenyl)-N-(2- | | | | 1H), 8.44 (d, J = 2.4 Hz, |
| | (1,2-dihydroxyethyl)-5- | | | | 1H), 8.15 (d, J = 6.5 Hz, |
| | fluoropyridin-4-yl)-5- | | | | 1H), 7.22-7.16 (m, 2H), |
| | methyl-5- | | | | 5.45 (d, J = 4.8 Hz, 1H), |
| | (trifluoromethyl)tetrahydrofuran-2- | | | | 4.86 (d, J = 10.1 Hz, 1H), |
| | carboxamide | | | | 4.65 (s, 1H), 4.55-4.52 |
| | (derived from | | | | (m, 1H), 4.06 (q, J = 10.1 |
| | Intermediate M, second | | | | Hz, 1H), 3.63 (d, J = 11.2 |
| | eluting isomer; second | | | | Hz, 1H), 3.46-3.42 (m, |
| | eluting isomer in SFC | | | | 1H), 3.88 (d, J = 2.0 Hz, |
| | separation (Step 9)) | | | | 3H), 1.56 (s, 3H) ppm. 2 × |
| | | | | | H hidden by DMSO. |
| 693 | rel-(2S,3R,5S)-3-(3,4- | 490.42 | 491 | 2.98 | ¹H NMR (500 MHz, |
| | difluoro-2- | | | | DMSO-d₆) δ 10.30 (s, |
| | methoxyphenyl)-N-(2- | | | | 1H), 8.34 (d, J = 5.6 Hz, |
| | (anti-1,2- | | | | 1H), 7.67 (d, J = 1.9 Hz, |
| | dihydroxypropyl)pyridin- | | | | 1H), 7.54 (dd, J = 5.5, |
| | 4-yl)-5-methyl-5- | | | | 2.1 Hz, 1H), 7.19-7.17 |
| | (trifluoromethyl)tetrahydrofuran-2- | | | | (m, 2H), 5.27 (d, J = 4.8 |
| | carboxamide | | | | Hz, 1H), 4.66 (s, 1H), |
| | (first eluting isomer in | | | | 4.64 (d, J = 3.9 Hz, 1H), |
| | SFC separation (Step 9)) | | | | 4.42 (t, J = 4.8 Hz, 1H), |
| | | | | | 4.06 (q, J = 10.1 Hz, 1H), |
| | | | | | 3.90-3.86 (m, 1H), 3.86 |
| | | | | | (d, J = 2.0 Hz, 3H), 2.46 |
| | | | | | (d, J = 10.1 Hz, 2H), 1.56 |
| | | | | | (s, 3H), 0.92 (d, J = 6.3 |
| | | | | | Hz, 3H) ppm. |
| 694 | rel-(2R,3S,5R)-3-(3,4- | 490.42 | 490.9 | 2.97 | ¹H NMR (500 MHz, |
| | difluoro-2- | | | | DMSO-d₆) δ 10.29 (s, |
| | methoxyphenyl)-N-(2- | | | | 1H), 8.34 (d, J = 5.6 Hz, |
| | (anti-1,2- | | | | 1H), 7.68-7.67 (m, 1H), |
| | dihydroxypropyl)pyridin- | | | | 7.53 (dd, J = 5.5, 2.2 Hz, |
| | 4-yl)-5-methyl-5- | | | | 1H), 7.19-7.17 (m, 2H), |
| | (trifluoromethyl)tetrahydrofuran-2- | | | | 5.27 (d, J = 4.7 Hz, 1H), |
| | carboxamide | | | | 4.66 (s, 1H), 4.64 (d, J = |
| | (second eluting isomer | | | | 4.2 Hz, 1H), 4.42 (t, J = |
| | in SFC separation (Step 9)) | | | | 5.1 Hz, 1H), 4.09-4.03 |
| | | | | | (m, 1H), 3.90-3.87 (m, |
| | | | | | 1H), 3.86 (d, J = 2.0 Hz, |
| | | | | | 3H), 2.46 (d, J = 10.6 Hz, |
| | | | | | 2H), 1.56 (s, 3H), 0.91 |
| | | | | | (d, J = 6.4 Hz, 3H) ppm. |
| 695 | rel-(2S,3R,5S)-3-(3,4- | 490.42 | 491 | 2.97 | |
| | difluoro-2- | | | | |
| | methoxyphenyl)-N-(2- | | | | |
| | (anti-1,2- | | | | |
| | dihydroxypropyl)pyridin- | | | | |
| | 4-yl)-5-methyl-5- | | | | |
| | (trifluoromethyl)tetrahydrofuran-2- | | | | |
| | carboxamide | | | | |
| | (third eluting isomer in | | | | |
| | SFC separation (Step 9)) | | | | |
| 696 | rel-(2R,3S,5R)-3-(3,4- | 490.42 | 491 | 2.97 | |
| | difluoro-2- | | | | |
| | methoxyphenyl)-N-(2- | | | | |
| | (anti-1,2- | | | | |
| | dihydroxypropyl)pyridin- | | | | |
| | 4-yl)-5-methyl-5- | | | | |
| | (trifluoromethyl)tetrahydrofuran-2- | | | | |
| | carboxamide | | | | |
| | (fourth eluting isomer | | | | |
| | in SFC separation (Step 9)) | | | | |
| 697 | rel-(2S,3R,5S)-3-(3,4- | 490.42 | 491 | 2.96 | |
| | difluoro-2- | | | | |
| | methoxyphenyl)-N-(2- | | | | |
| | (syn-1,2- | | | | |
| | dihydroxypropyl)pyridin- | | | | |
| | 4-yl)-5-methyl-5- | | | | |
| | (trifluoromethyl)tetrahydrofuran-2- | | | | |
| | carboxamide | | | | |
| | (Peak 1A in SFC | | | | |
| | separation (Step 9)) | | | | |
| 698 | rel-(2S,3R,5S)-3-(3,4- | 490.42 | 491 | 2.96 | |
| | difluoro-2- | | | | |
| | methoxyphenyl)-N-(2- | | | | |
| | ((1R,2S)-1,2- | | | | |
| | dihydroxypropyl)pyridin- | | | | |
| | 4-yl)-5-methyl-5- | | | | |
| | (trifluoromethyl)tetrahydrofuran-2- | | | | |
| | carboxamide | | | | |
| | (Peak 1B in SFC | | | | |
| | separation (Step 9)) | | | | |
| 699 | rel-(2R,3S,5R)-3-(3,4- | 490.42 | 491 | 2.96 | |
| | difluoro-2- | | | | |
| | methoxyphenyl)-N-(2- | | | | |
| | ((1S,2R)-1,2- | | | | |
| | dihydroxypropyl)pyridin- | | | | |
| | 4-yl)-5-methyl-5- | | | | |
| | (trifluoromethyl)tetrahydrofuran-2- | | | | |
| | carboxamide | | | | |
| | (Peak 2 in SFC | | | | |
| | separation (Step 9)) | | | | |
| 700 | rel-(2R,3S,5R)-3-(3,4- | 490.42 | 491 | 2.96 | |
| | difluoro-2- | | | | |
| | methoxyphenyl)-N-(2- | | | | |
| | ((1R,2S)-1,2- | | | | |
| | dihydroxypropyl)pyridin- | | | | |
| | 4-yl)-5-methyl-5- | | | | |
| | (trifluoromethyl)tetrahydrofuran-2- | | | | |
| | carboxamide | | | | |
| | (Peak 3 in SFC | | | | |
| | separation (Step 9)) | | | | |
| 701 | rel-(2R,3S,5R)-3-(3,4- | 490.42 | 491.5 | 95.9 | ¹H NMR (500 MHz, |
| | difluoro-2- | | | | DMSO-d₆) δ 10.30 (s, |
| | methoxyphenyl)-N-(2- | | | | 1H), 8.35 (d, J = 5.5 Hz, |
| | (1,2-dihydroxypropan- | | | | 1H), 7.80 (d, J = 2.0 Hz, |
| | 2-yl)pyridin-4-yl)-5- | | | | 1H), 7.55 (dd, J = 5.5, |
| | methyl-5- | | | | 2.1 Hz, 1H), 7.19-7.17 |
| | (trifluoromethyl)tetrahydrofuran-2- | | | | (m, 2H), 5.03 (s, 1H), |
| | carboxamide | | | | 4.67 (d, J = 10.1 Hz, 1H), |
| | (first eluting isomer in | | | | 4.59 (t, J = 6.0 Hz, 1H), |
| | SFC separation (Step 9)) | | | | 4.06 (q, J = 9.9 Hz, 1H), |
| | | | | | 3.87 (d, J = 2.0 Hz, 3H), |
| | | | | | 3.51 (d, J = 6.1 Hz, 2H), |
| | | | | | 2.46 (d, J = 10.0 Hz, 2H), |
| | | | | | 1.56 (s, 3H), 1.34 (s, 3H) |
| | | | | | ppm. |
| 702 | rel-(2R,3S,5R)-3-(3,4- | 490.42 | 491.5 | 2.95 | ¹H NMR (500 MHz, |
| | difluoro-2- | | | | DMSO-d₆) δ 10.30 (s, |
| | methoxyphenyl)-N-(2- | | | | 1H), 8.35 (d, J = 5.3 Hz, |
| | (1,2-dihydroxypropan- | | | | 1H), 7.80 (d, J = 1.5 Hz, |
| | 2-yl)pyridin-4-yl)-5- | | | | 1H), 7.55 (dd, J = 5.5, |
| | methyl-5- | | | | 2.1 Hz, 1H), 7.19-7.15 |
| | (trifluoromethyl)tetrahydrofuran-2- | | | | (m, 2H), 5.04 (s, 1H), |
| | carboxamide | | | | 4.67 (d, J = 10.1 Hz, 1H), |
| | (second eluting isomer | | | | 4.58 (t, J = 6.0 Hz, 1H), |
| | in SFC separation (Step 9)) | | | | 4.06 (q, J = 10.0 Hz, 1H), |
| | | | | | 3.87 (d, J = 2.1 Hz, 3H), |
| | | | | | 3.50 (d, J = 6.1 Hz, 2H), |
| | | | | | 2.46 (d, J = 10.0 Hz, 2H), |
| | | | | | 1.56 (s, 3H), 1.34 (s, 3H) |
| | | | | | ppm. |

The following compounds were made using a similar method to that described in Example 26, except that (4-fluoro-2-methoxy-3-methylphenyl)boronic acid was used in step 5 and 2-(2,2-dimethyl-1,3-dioxolan-4-yl)pyridin-4-amine (first of second eluting isomer by SFC) was used in the amide coupling step 8. The separated isomers generated in step 9 were deprotected using General Method B.

| | | | | MS | |
|---|---|---|---|---|---|
| Cmpd | | LC/MS | Found | retention | |
| No. | Compound Name | (m/z calc.) | M + 1 | time | NMR (shifts in ppm) |
| 703 | rel-(2S,3R,5S)-N-(2- | 472.43 | 473.5 | 2.87 | ¹H NMR (400 MHz, |
| | ((S)-1,2- | | | | DMSO-d₆) δ 10.32 (s, |
| | dihydroxyethyl)pyridin- | | | | 1H), 8.32 (d, J = 5.5 Hz, |
| | 4-yl)-3-(4-fluoro-2- | | | | 1H), 7.65 (d, J = 2.1 Hz, |
| | methoxy-3- | | | | 1H), 7.52 (dd, J = 5.6, |
| | methylphenyl)-5- | | | | 2.1 Hz, 1H), 7.25 (dd, J = |
| | methyl-5- | | | | 8.7, 6.5 Hz, 1H), 7.02 (t, |
| | (trifluoromethyl)tetrahydrofuran-2- | | | | J = 8.8 Hz, 1H), 5.37 (d, |
| | carboxamide | | | | J = 4.7 Hz, 1H), 4.70- |
| | (precursor was first | | | | 4.61 (m, 2H), 4.57-4.48 |
| | eluting isomer by SFC | | | | (m, 1H), 4.16-4.04 (m, |
| | using Whelk-O1 | | | | 1H), 3.69-3.59 (m, 4H), |
| | column; first eluting | | | | 3.47-3.38 (m, 1H), 2.49- |
| | isomer (by SFC) of 2- | | | | 2.44 (m, 1H), 2.31 (t, J = |
| | (2,2-dimethyl-1,3- | | | | 12.4 Hz, 1H), 2.11 (d, J = |
| | dioxolan-4-yl)pyridin- | | | | 2.1 Hz, 3H), 1.56 (s, 3H) |
| | 4-amine used in step 8) | | | | ppm. |
| 704 | rel-(2R,3S,5R)-N-(2- | 472.43 | 473.5 | 2.87 | |
| | ((S)-1,2- | | | | |
| | dihydroxyethyl)pyridin- | | | | |
| | 4-yl)-3-(4-fluoro-2- | | | | |
| | methoxy-3- | | | | |
| | methylphenyl)-5- | | | | |
| | methyl-5- | | | | |
| | (trifluoromethyl)tetrahydrofuran-2- | | | | |
| | carboxamide | | | | |
| | (precursor was second | | | | |
| | eluting isomer by SFC | | | | |
| | using Whelk-O1 | | | | |
| | column; first eluting | | | | |
| | isomer (by SFC) of 2- | | | | |
| | (2,2-dimethyl-1,3- | | | | |
| | dioxolan-4-yl)pyridin- | | | | |
| | 4-amine used in step 8) | | | | |
| 705 | rel-(2S,3R,5S)-N-(2- | 472.43 | 473.5 | 2.87 | |
| | (1,2- | | | | |
| | dihydroxyethyl)pyridin- | | | | |
| | 4-yl)-3-(4-fluoro-2- | | | | |
| | methoxy-3- | | | | |
| | methylphenyl)-5- | | | | |
| | methyl-5- | | | | |
| | (trifluoromethyl)tetrahydrofuran-2- | | | | |
| | carboxamide | | | | |
| | (precursor was first | | | | |
| | eluting isomer by SFC | | | | |
| | using Whelk-O1 | | | | |
| | column; second eluting | | | | |
| | isomer (by SFC) of 2- | | | | |
| | (2,2-dimethyl-1,3- | | | | |
| | dioxolan-4-yl)pyridin- | | | | |
| | 4-amine used in step 8) | | | | |
| 706 | rel-(2R,3S,5R)-N-(2- | 472.43 | 473.5 | 2.87 | |
| | ((R)-1,2- | | | | |
| | dihydroxyethyl)pyridin- | | | | |
| | 4-yl)-3-(4-fluoro-2- | | | | |
| | methoxy-3- | | | | |
| | methylphenyl)-5- | | | | |
| | methyl-5- | | | | |
| | (trifluoromethyl)tetrahydrofuran-2- | | | | |
| | carboxamide | | | | |
| | (precursor was second | | | | |
| | eluting isomer by SFC | | | | |
| | using Whelk-O1 | | | | |
| | column; second eluting | | | | |
| | isomer (by SFC) of 2- | | | | |
| | (2,2-dimethyl-1,3- | | | | |
| | dioxolan-4-yl)pyridin- | | | | |
| | 4-amine used in step 8) | | | | |

The following compounds were made using a similar method to that described in Example 26, except that (2-ethoxy-3,4-difluorophenyl)boronic acid was used in step 5 and 3-methoxy-[1,2,4]triazolo[4,3-a]pyridin-7-amine was used in the amide coupling step 8. Chiral SFC separation in step 9 used a (R′R) Whelk 01 column. 3-5 μm particle size, 5.0 cm×3.0 mm from Regis Technologies with Solvent A: 600% liquid CO₂; Solvent B: 400% MeOH: MeCN (1:1) with 0.20% DMIPA.

| | | | | MS | |
|---|---|---|---|---|---|
| Cmpd | | LC/MS | Found | retention | |
| No. | Compound Name | (m/z calc.) | M + 1 | time | NMR (shifts in ppm) |
| 707 | rel-(2S,3R,5S)-3-(2- | 500.419 | | 2.94 | ¹H NMR (500 MHz, |
| | ethoxy-3,4- | | | | DMSO-d₆) δ 10.58 (s, |
| | difluorophenyl)-N-(3- | | | | 1H), 8.10-8.02 (m, 1H), |
| | methoxy- | | | | 8.02-7.93 (m, 1H), 7.25- |
| | [1,2,4]triazolo[4,3- | | | | 7.09 (m, 2H), 6.95 (dd, |
| | α]pyridin-7-yl)-5- | | | | J = 7.4, 1.8 Hz, 1H), 4.69 |
| | methyl-5- | | | | (d, J = 10.1 Hz, 1H), 4.21- |
| | (trifluoromethyl)tetrahydrofuran-2- | | | | 4.01 (m, 3H), 3.64 (s, |
| | carboxamide | | | | 3H), 2.48-2.42 (m, 2H), |
| | (first eluting isomer) | | | | 1.56 (s, 3H), 1.29 (t, J = |
| | | | | | 7.0 Hz, 3H) ppm. |
| 708 | rel-((2R,3S,5R)-3-(2- | 500.419 | 501.3 | 2.94 | |
| | ethoxy-3,4- | | | | |
| | difluorophenyl)-N-(3- | | | | |
| | methoxy- | | | | |
| | [1,2,4]triazolo[4,3- | | | | |
| | α]pyridin-7-yl)-5- | | | | |
| | methyl-5- | | | | |
| | (trifluoromethyl)tetrahydrofuran-2- | | | | |
| | carboxamide | | | | |
| | (second eluting isomer) | | | | |

The following compounds were made using a similar method to that described in Example 26, except that (2-ethoxy-3,4-difluorophenyl)boronic acid was used in step 5 and 2-(2,2-dimethyl-1,3-dioxolan-4-yl)pyridin-4-amine (first or second eluting isomer by SFC) was used in the amide coupling step 8. The separated isomers generated in step 9 were deprotected using General Method B.

| | | | | MS | |
|---|---|---|---|---|---|
| Cmpd | | LC/MS | Found | retention | |
| No. | Compound Name | (m/z calc.) | M + 1 | time | NMR (shifts in ppm) |
| 709 | rel-(2S,3R,5S)-N-(2- | 490.42 | 491.5 | 2.98 | |
| | (1,2- | | | | |
| | dihydroxyethyl)pyridin- | | | | |
| | 4-yl)-3-(2-ethoxy-3,4- | | | | |
| | difluorophenyl)-5- | | | | |
| | methyl-5- | | | | |
| | (trifluoromethyl)tetrahydrofuran-2- | | | | |
| | carboxamide | | | | |
| | (precursor was first | | | | |
| | eluting isomer by SFC | | | | |
| | using Whelk-O1 | | | | |
| | column; first eluting | | | | |
| | isomer (by SFC) of 2- | | | | |
| | (2,2-dimethyl-1,3- | | | | |
| | dioxolan-4-yl)pyridin- | | | | |
| | 4-amine used in step 8) | | | | |
| 710 | rel-(2R,3S,5R)-N-(2- | 490.42 | 491.4 | 2.98 | ¹H NMR (500 MHz, |
| | (1,2- | | | | DMSO-d₆) δ 10.27 (s, |
| | dihydroxyethyl)pyridin- | | | | 1H), 8.33 (d, J = 5.5 Hz, |
| | 4-yl)-3-(2-ethoxy-3,4- | | | | 1H), 7.68 (d, J = 2.2 Hz, |
| | difluorophenyl)-5- | | | | 1H), 7.52 (dd, J = 5.6, |
| | methyl-5- | | | | 2.1 Hz, 1H), 7.24-7.12 |
| | (trifluoromethyl)tetrahydrofuran-2- | | | | (m, 2H), 5.37 (d, J = 4.6 |
| | carboxamide | | | | Hz, 1H), 4.70-4.62 (m, |
| | (precursor was second | | | | 2H), 4.56-4.49 (m, 1H), |
| | eluting isomer by SFC | | | | 4.20-3.98 (m, 3H), 3.65 |
| | using Whelk-O1 | | | | (ddd, J = 10.9, 6.0, 4.0 |
| | column; first eluting | | | | Hz, 1H), 3.42 (ddd, J = |
| | isomer (by SFC) of 2- | | | | 11.0, 6.9, 5.9 Hz, 1H), |
| | (2,2-dimethyl-1,3- | | | | 2.48-2.41 (m, 2H), 1.56 |
| | dioxolan-4-yl)pyridin- | | | | (s, 3H), 1.28 (t, J = 7.0 |
| | 4-amine used in step 8) | | | | Hz, 3H) ppm. |
| 711 | rel-(2R,3S,5R)-N-(2- | 490.42 | 491.3 | 2.96 | |
| | (1,2- | | | | |
| | dihydroxyethyl)pyridin- | | | | |
| | 4-yl)-3-(2-ethoxy-3,4- | | | | |
| | difluorophenyl)-5- | | | | |
| | methyl-5- | | | | |
| | (trifluoromethyl)tetrahydrofuran-2- | | | | |
| | carboxamide | | | | |
| | (precursor was first | | | | |
| | eluting isomer by SFC | | | | |
| | using Whelk-O1 | | | | |
| | column; second eluting | | | | |
| | isomer (by SFC) of 2- | | | | |
| | (2,2-dimethyl-1,3- | | | | |
| | dioxolan-4-yl)pyridin- | | | | |
| | 4-amine used in step 8) | | | | |
| 712 | rel-(2S,3R,5S)-N-(2- | 490.42 | 491.6 | 2.98 | |
| | (1,2- | | | | |
| | dihydroxyethyl)pyridin- | | | | |
| | 4-yl)-3-(2-ethoxy-3,4- | | | | |
| | difluorophenyl)-5- | | | | |
| | methyl-5- | | | | |
| | (trifluoromethyl)tetrahydrofuran-2- | | | | |
| | carboxamide | | | | |
| | (precursor was second | | | | |
| | eluting isomer by SFC | | | | |
| | using Whelk-O1 | | | | |
| | column; second eluting | | | | |
| | isomer (by SFC) of 2- | | | | |
| | (2,2-dimethyl-1,3- | | | | |
| | dioxolan-4-yl)pyridin- | | | | |
| | 4-amine used in step 8) | | | | |

### Example 27

rel-(2S,3R,5S)-3-(4-fluoro-2-methoxyphenyl)-5-methyl-N-(2-(methylsulfonyl)pyridin-4-yl)-5-(trifluoromethyl)tetrahydrofuran-2-carboxamide (713) and rel-(2R,3S,5R)-3-(4-fluoro-2-methoxyphenyl)-5-methyl-N-(2-(methylsulfonyl)pyridin-4-yl)-5-(trifluoromethyl)tetrahydrofuran-2-carboxamide (714)

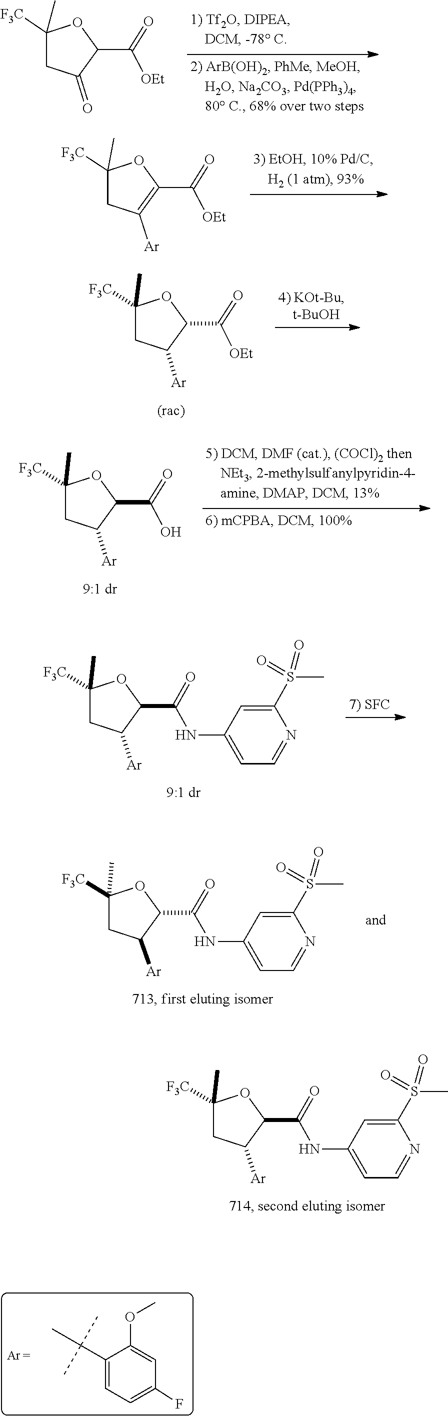

### Step 1:

Triflic anhydride (1.5 mL, 8.92 mmol) was added dropwise to a solution of ethyl 5-methyl-3-oxo-5-(trifluoromethyl)tetrahydrofuran-2-carboxylate (1.66 g, 6.91 mmol) and DIPEA (3.6 mL, 20.67 mmol) in DCM (50 mL), and the reaction was stirred at −78° C. After 3 hours saturated aqueous NaHCO₃ was added, the layers were separated, and the aqueous layer was extracted with DCM. The combined organic layers were dried (MgSO₄), filtered, and concentrated in vacuo to give ethyl 2-methyl-2-(trifluoromethyl)-4-(trifluoromethylsulfonyloxy)-3H-furan-5-carboxylate (2.573 g), which was used in the next step without further purification.

### Step 2:

A solution of ethyl 2-methyl-2-(trifluoromethyl)-4-(trifluoromethylsulfonyloxy)-3H-furan-5-carboxylate (2.573 g, 6.91 mmol), (4-fluoro-2-methoxy-phenyl)boronic acid (1.84 g, 10.83 mmol), Pd(PPh₃)₄(600 mg, 0.52 mmol), and Na₂CO₃ (1.8 g, 16.98 mmol) in PhMe (30 mL), MeOH (3 mL) and H₂O (3 mL) was degassed, then heated at 80° C. for 16 hours. The reaction was cooled to ambient temperature, diluted with EtOAc, the layers separated, and the organic layer washed with brine, dried (MgSO₄), filtered, and concentrated in vacuo. Purification by flash chromatography (80 g SiO₂, 0 to 20% EtOAc in petrol) gave ethyl 4-(4-fluoro-2-methoxy-phenyl)-2-methyl-2-(trifluoromethyl)-3H-furan-5-carboxylate (1.64 g, 68% over two steps) as a yellow oil. ESI-MS m/z calc. 348.09848, found 349.2 (M+1)⁺.

### Step 3:

EtOH (45 mL) was added to a flask containing ethyl 4-(4-fluoro-2-methoxy-phenyl)-2-methyl-2-(trifluoromethyl)-3H-furan-5-carboxylate (1.64 g, 4.71 mmol) and Pd/C (500 mg, 0.47 mmol). The mixture was degassed then stirred under a balloon of hydrogen for 24 hours. The mixture was filtered through Celite, washing with EtOH, and concentrated in vacuo. Pd/C (500 mg, 0.47 mmol) was added to the residue, and the mixture was suspended in EtOH (45 mL). The mixture was degassed then stirred under a balloon of hydrogen for 24 hours. The mixture was filtered through Celite, washing with EtOH, and concentrated in vacuo to give ethyl rac-(2S,3S,5R)-3-(4-fluoro-2-methoxy-phenyl)-5-methyl-5-(trifluoromethyl)tetrahydrofuran-2-carboxylate (1.53 g, 93%) as an off-white solid. ESI-MS m/z calc. 350.11414, found 351.2 (M+1)⁺.

### Step 4:

A solution of ethyl rac-(2S,3S,5R)-3-(4-fluoro-2-methoxy-phenyl)-5-methyl-5-(trifluoromethyl)tetrahydrofuran-2-carboxylate (10 mg, 0.029 mmol), and KOt-Bu (192 mg, 1.711 mmol) in tert-butanol (0.3 mL) was stirred at ambient temperature for 30 mins. The reaction was diluted in EtOAc and quenched by addition of a saturated aqueous NH₄Cl solution. This process was repeated a further 19 times and the 20 reactions combined for the rest of the work-up. The layers were separated and the aqueous layer extracted with EtOAc. The aqueous layer was then acidified with 1 M HCl and extracted again. The combined organic layers were dried (MgSO₄), filtered and concentrated in vacuo to give rac-(2R,3S,5R)-3-(4-fluoro-2-methoxy-phenyl)-5-methyl-5-(trifluoromethyl)tetrahydrofuran-2-carboxylic acid (186 mg) as an orange residue, in an -89:11 diastereomeric ratio, which was taken on to the next step without further purification.

### Step 5:

To a solution of rac-(2R,3S,5R)-3-(4-fluoro-2-methoxy-phenyl)-5-methyl-5-(trifluoromethyl)tetrahydrofuran-2-carboxylic acid (256 mg, 0.7944 mmol) in DCM (9 mL) was added DMF (10 μL, 0.1291 mmol) and oxalyl chloride (210 μL, 2.407 mmol) at ambient temperature. Gas evolution was observed. After 30 mins, the reaction mixture was concentrated in vacuo then diluted in DCM (6 mL) and added dropwise to a solution of 2-methylsulfanylpyridin-4-amine (Hydrochloride salt) (210 mg, 1.189 mmol) and Et₃N (700 μL, 5.022 mmol) in DCM (3 mL) at 0° C. DMAP (10 mg, 0.08185 mmol) was added, the reaction was warmed to ambient temperature after 10 minutes and stirred for 16 hours. The reaction mixture was diluted with DCM and washed with 2 M HCl solution, dried (MgSO₄), filtered, and concentrated in vacuo. Purification by flash column chromatography gave (40 g SiO₂, 0 to 100% ethylacetate in petroleum ether) gave rac-(2R,3S,5R)-3-(4-fluoro-2-methoxyphenyl)-5-methyl-N-(2-(methylthio)pyridin-4-yl)-5-(trifluoromethyl)tetrahydrofuran-2-carboxamide (47 mg, 13%). ESI-MS m/z calc. 444.11307, found 445.1 (M+1)⁺; Retention time: 0.99 minutes as a white solid.

### Step 6:

mCPBA (80 mg, 0.3245 mmol) was added to a solution of rac-(2R,3S,5R)-3-(4-fluoro-2-methoxyphenyl)-5-methyl-N-(2-(methylthio)pyridin-4-yl)-5-(trifluoromethyl)tetrahydrofuran-2-carboxamide (47 mg, 0.1058 mmol) in dichloromethane (1.0 mL) at 0° C. The reaction mixture was warmed to ambient temperature after 30 minutes and stirred for 16 hours. The reaction was diluted with dichloromethane and washed with saturated aqueous NaHCO₃ solution. The organics were dried (MgSO₄), filtered, and concentrated in vacuo to afford rac-(2R,3S,5R)-3-(4-fluoro-2-methoxyphenyl)-5-methyl-N-(2-(methylsulfonyl)pyridin-4-yl)-5-(trifluoromethyl)tetrahydrofuran-2-carboxamide (50 mg, 100%) as a white solid. ESI-MS m/z calc. 476.1029, found 477.1 (M+1)⁺; 475.5 (M−1); Retention time: 0.89 minutes.

### Step 7:

The enantiomers of rac-(2R,3S,5R)-3-(4-fluoro-2-methoxyphenyl)-5-methyl-N-(2-(methylsulfonyl)pyridin-4-yl)-5-(trifluoromethyl)tetrahydrofuran-2-carboxamide (50 mg, 0.1049 mmol) were separated by chiral SFC using a (R,R)-Whelk-01 column.

First eluting isomer (rt=1.61 minutes): rel-(2S,3R,5S)-3-(4-fluoro-2-methoxyphenyl)-5-methyl-N-(2-(methylsulfonyl)pyridin-4-yl)-5-(trifluoromethyl)tetrahydrofuran-2-carboxamide (713, 8.4 mg, 33%). ¹H NMR (500 MHz, DMSO-d₆) δ 10.64 (s, 1H), 8.63 (dd, J=5.5, 0.6 Hz, 1H), 8.36 (d, J=2.1 Hz, 1H), 7.87 (dd, J=5.5, 2.1 Hz, 1H), 7.33 (dd, J=8.5, 6.7 Hz, 1H), 6.90 (dd, J=11.3, 2.5 Hz, 1H), 6.76 (td, J=8.4, 2.6 Hz, 1H), 4.65 (d, J=10.0 Hz, 1H), 4.06-3.95 (m, 1H), 3.69 (s, 3H), 3.25 (s, 3H), 2.62 (t, J=12.2 Hz, 1H), 2.35 (dd, J=12.6, 8.0 Hz, 1H), 1.55 (s, 3H) ppm. ESI-MS m/z calc. 476.1029, found 477.6 (M+1)⁺; 475.6 (M−1)⁻; Retention time: 3.13 minutes.

Second eluting isomer (rt=1.86 minutes): rel-(2R,3S,5R)-3-(4-fluoro-2-methoxyphenyl)-5-methyl-N-(2-(methylsulfonyl)pyridin-4-yl)-5-(trifluoromethyl)tetrahydrofuran-2-carboxamide (714, 10.1 mg, 40%) ESI-MS m/z calc. 476.1029, found 477.6 (M+1)⁺; 475.6 (M−1)⁻; Retention time: 3.13 minutes.

The following compounds were made using a method similar to Example 27 except that General Method G, step 1 was used on rac-(2R,3S,5R)-3-(4-fluoro-2-methoxyphenyl)-5-methyl-N-(2-(methylthio)pyridin-4-yl)-5-(trifluoromethyl)tetrahydrofuran-2-carboxamide (product of step 5) and steps 6 and 7 were omitted. The product of General Method G, step 1 was purified by achiral SFC using a Chiralpak IA column and eluting with a gradient of 20 mM NH₃ in MeOH to provide a mixture of 4 stereoisomers (two pairs of enantiomers that are epimeric at the sulfonimidoyl position):

| | | | | | |
|---|---|---|---|---|---|
| | | | | MS | |
| Cmpd | | LC/MS | Found | retention | |
| No. | Compound Name | (m/z calc.) | M + 1 | time | NMR (shifts in ppm) |

| | | | | |
|---|---|---|---|---|
| 715 | 3-(4-fluoro-2-methoxy- | 475.457 | 476.1 | 2.86 |
| | phenyl)-5-methyl-N- | | | |
| | [2- | | | |
| | (methylsulfonimidoyl)- | | | |
| | 4-pyridyl]-5- | | | |
| | (trifluoromethyl)tetrahydrofuran-2- | | | |
| | carboxamide | | | |

### Example 28

rel-(2S,3R,5S)-3-(3-ethyl-4-fluoro-2-methoxyphenyl)-5-methyl-N-(3-methyl-1-(methylsulfonyl)-1H-pyrazol-4-yl)-5-(trifluoromethyl)tetrahydrofuran-2-carboxamide (716) and rel-(2R,3S,5R)-3-(3-ethyl-4-fluoro-2-methoxyphenyl)-5-methyl-N-(3-methyl-1-(methylsulfonyl)-1H-pyrazol-4-yl)-5-(trifluoromethyl)tetrahydrofuran-2-carboxamide (717)

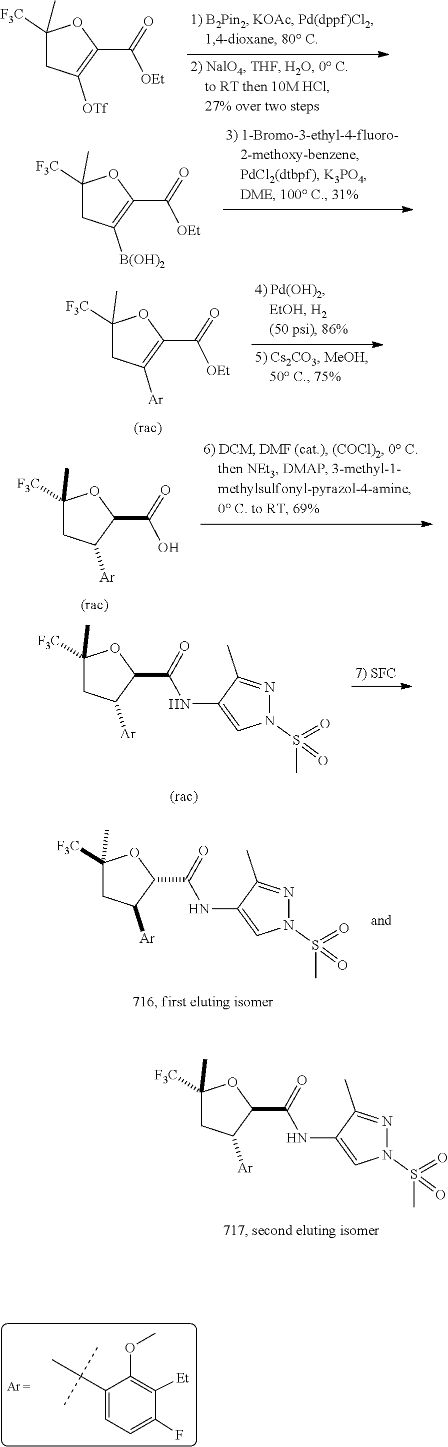

### Step 1 and 2:

To a stirred solution of ethyl 2-methyl-2-(trifluoromethyl)-4-(trifluoromethylsulfonyloxy)-3H-furan-5-carboxylate (10 g, 26.865 mmol) in 1,4-dioxane (150 mL), potassium acetate (8 g, 81.514 mmol) and 4,4,5,5-tetramethyl-2-(4,4,5,5-tetramethyl-1,3,2-dioxaborolan-2-yl)-1,3,2-dioxaborolane (27 g, 106.32 mmol) were added. The reaction mixture was de-gassed by argon gas then Pd(dppf)Cl₂ (983 mg, 1.3434 mmol) was added to this reaction mixture and heated to 80° C. under argon for 15 min. The reaction was monitored by TLC and allowed to reach room temperature. The reaction was then diluted with EtOAc (500 mL) and water (300 mL) and filtered through a celite bed. The two layers were separated and the aqueous layer was extracted with EtOAc (300 mL). The combined organic layers were dried over anhydrous sodium sulfate and concentrated under vacuum.

The crude product was dissolved in THF (30 mL) and H₂O (15 mL) and cooled to 0-5° C. NaIO₄ (17 g, 79.479 mmol) was added in the reaction mixture portion wise and stirred it at room temperature for 15 min. Then HCl (10 mL of 1 M, 10.000 mmol) was added and the reaction mass was stirred for 4 hours. The reaction mass was then diluted with water (200 mL) and EtOAc (500 mL). The layers were separated and the organic layer was washed with brine. The organic layer was dried over anhydrous sodium sulfate and concentrated under reduced pressure. The crude mass was purified by silica gel chromatography using 2-10% EtOAc in hexane to get a yellow solid, which was washed with hexane to get [5-ethoxycarbonyl-2-methyl-2-(trifluoromethyl)-3H-furan-4-yl]boronic acid (2.5 g, 27%) as a white solid. ¹H NMR (400 MHz, DMSO-d6) δ 8.22 (s, 2H), 4.23 (q, J=7.1 Hz, 2H), 3.11 (d, J=18.2 Hz, 1H), 2.85 (d, J=18.24 Hz, 1H), 1.49 (s, 3H), 1.25 (t, J=7.1 Hz, 3H) ppm.

### Step 3:

To a stirred solution of 1-bromo-3-ethyl-4-fluoro-2-methoxy-benzene (3 g, 12.87 mmol) and [5-ethoxycarbonyl-2-methyl-2-(trifluoromethyl)-3H-furan-4-yl]boronic acid (3.79 g, 14.16 mmol) in DME (15 mL) was added K₃PO₄ (7.65 g, 36.04 mmol). The mixture was degassed with N₂ gas for 5 mins followed by addition of PdCl₂(dtbpf) (838.86 mg, 1.29 mmol) and heated to 100° C. for 4 h. The reaction mixture was filtered through a celite pad, the filtrate was diluted with water (50 mL) and the aqueous layer extracted with EtOAc (100 mL). The organic layer was dried (MgSO₄), filtered and evaporated in vacuo. Purification by flash chromatography (SiO₂, 0 to 3% EtOAc in hexane) gave ethyl 4-(3-ethyl-4-fluoro-2-methoxy-phenyl)-2-methyl-2-(trifluoromethyl)-3H-furan-5-carboxylate (1.5 g, 31%) as light yellow liquid. ¹H NMR (400 MHz, DMSO-d₆) δ 7.1823 (t, J=7 Hz, 1H), 6.9648 (t, J=8.8 Hz, 1H) 4.0401 (q, J=6.9 Hz, 2H), 3.5996 (s, 3H), 3.4321 (d, J=17.6 Hz, 1H), 3.1492 (d, J=17.6 Hz, 1H), 2.6211-2.5858 (m, 2H), 1.614 (s, 3H), 1.1339 (t, J=7.3 Hz, 3H), 1.0159 (t, J=7 Hz, 3H) ppm. ESI-MS m/z calc. 376.1298, found 377.0 (M+1).

### Step 4:

To a stirred solution of ethyl 4-(3-ethyl-4-fluoro-2-methoxy-phenyl)-2-methyl-2-(trifluoromethyl)-3H-furan-5-carboxylate (1.5 g, 3.99 mmol) in ethanol (50 mL) was added Pd(OH)₂ (4.5 g, 32.04 mmol). The reaction was stirred at ambient temperature for 16 hours in a Parr shaker under a 50 psi pressure of hydrogen. Reaction mass was filtered through celite bed, filtrate was evaporated under reduced pressure to get crude compound. Purification by flash chromatography (SiO₂, 30% EtOAc in hexane) gave ethyl rac-(2S,3S,5R)-3-(3-ethyl-4-fluoro-2-methoxy-phenyl)-5-methyl-5-(trifluoromethyl)tetrahydrofuran-2-carboxylate (1.3 g, 86%) as a white solid. ¹H NMR (400 MHz, DMSO-d₆) δ 7.04 (t, J=6.9 Hz, 1H), 6.92 (t, J=9 Hz, 1H) 4.95 (d, J=8.9 Hz, 1H), 4.35-4.28 (m, 1H), 3.80 (s, 3H), 3.70-3.59 (m, 2H), 2.63-2.54 (m, 3H), 2.31-2.26 (m, 1H), 1.49 (s, 3H), 1.13 (t, J=7.3 Hz, 3H), 0.72 (t, J=7 Hz, 3H) ppm. ESI-MS m/z calc. 378.1454, found 379.0 (M+1)⁺.

### Step 5:

To a stirred solution of ethyl rac-(2S,3S,5R)-3-(3-ethyl-4-fluoro-2-methoxy-phenyl)-5-methyl-5-(trifluoromethyl)tetrahydrofuran-2-carboxylate (1.2 g, 3.17 mmol) in MeOH (10 mL) was added cesium carbonate (2.07 g, 6.34 mmol). The reaction was stirred at an ambient temperature for 1h then it was heated at 50° C. for 16 h. The reaction mixture was concentrated in vacuo then water (5 mL) was added. The aqueous layer was acidified with 1M HCl, to neutral pH. The aqueous layer was extracted in 10% methanol-DCM mixture (2×50 mL) and the organic layer was evaporated in vacuo to give rac-(2R,3S,5R)-3-(3-ethyl-4-fluoro-2-methoxy-phenyl)-5-methyl-5-(trifluoromethyl)tetrahydrofuran-2-carboxylic acid (1 g, 75%) as a colourless liquid, which was used without further purification. ¹H NMR (400 MHz, DMSO-d₆) δ 12.89 (s, 1H), 7.29-7.25 (m, 1H) 6.99 (t, J=9 Hz, 1H), 4.44 (d, J=10.4, 1H), 3.91-3.88 (m, 1H), 3.70 (s, 3H), 2.64-2.58 (m, 2H), 2.49-2.42 (m, 1H), 2.27-1.98 (m, 1H), 1.48 (s, 3H), 1.14 (t, J=7.4 Hz, 3H) ppm. ESI-MS m/z calc. 350.1141, found 351.0 (M+1)⁺.

### Step 6:

To a solution of rac-(2R,3S,5R)-3-(3-ethyl-4-fluoro-2-methoxy-phenyl)-5-methyl-5-(trifluoromethyl)tetrahydrofuran-2-carboxylic acid (100 mg, 0.2855 mmol) in DCM (2.5 mL) was added DMF (5 μL, 0.06457 mmol) and carefully oxalyl chloride (100 μL, 1.146 mmol) at 0° C. Visible gas evolution. After 30 mins the reaction mixture was concentrated in vacuo, then diluted in DCM (2.3 mL) and added dropwise to a solution of 3-methyl-1-methylsulfonyl-pyrazol-4-amine (68 mg, 0.3881 mmol), DMAP (4 mg, 0.03274 mmol) and Et₃N (250 μL, 1.794 mmol) in DCM (2 mL) at 0° C. After 10 minutes the reaction was warmed to ambient temperature and stirred for 16 hours. The reaction mixture was then concentrated in vacuo directly onto silica gel. Purification by flash column chromatography (40 g SiO₂, 0 to 100% ethylacetate in heptane) gave rac-(2R,3S,5R)-3-(3-ethyl-4-fluoro-2-methoxyphenyl)-5-methyl-N-(3-methyl-1-(methylsulfonyl)-1H-pyrazol-4-yl)-5-(trifluoromethyl)tetrahydrofuran-2-carboxamide (100 mg, 69%). ESI-MS m/z calc. 507.1451, found 506.1 (M−1)⁻; Retention time: 0.98 minutes.

### Step 7:

The enantiomers of rac-(2R,3S,5R)-3-(3-ethyl-4-fluoro-2-methoxyphenyl)-5-methyl-N-(3-methyl-1-(methylsulfonyl)-1H-pyrazol-4-yl)-5-(trifluoromethyl)tetrahydrofuran-2-carboxamide (100 mg, 0.1970 mmol) were separated by chiral SFC using a Luc Cellulose-2 column, 5 μm particle size, 25 cm×10 mm from Phenomenex, Inc. on a Minigram SFC instrument from Berger Instruments.

First eluting isomer (rt=2.51 minutes): rel-(2S,3R,5S)-3-(3-ethyl-4-fluoro-2-methoxyphenyl)-5-methyl-N-(3-methyl-1-(methylsulfonyl)-1H-pyrazol-4-yl)-5-(trifluoromethyl)tetrahydrofuran-2-carboxamide (716, 28.2 mg, 56%). ¹H NMR (500 MHz, DMSO-d₆) δ 9.84 (s, 1H), 8.31 (s, 1H), 7.28 (dd, J=8.8, 6.4 Hz, 1H), 7.03 (t, J=9.0 Hz, 1H), 4.76 (d, J=10.1 Hz, 1H), 4.14-4.02 (m, 1H), 3.66 (s, 3H), 3.43 (s, 3H), 2.63-2.55 (m, 2H), 2.50-2.46 (m, 1H), 2.29 (t, J=12.4 Hz, 1H), 2.15 (s, 3H), 1.54 (s, 3H), 1.11 (t, J=7.5 Hz, 3H) ppm. ESI-MS m/z calc. 507.1451, found 508.2 (M+1)⁺; 506.2 (M−1)⁻; Retention time: 3.47 minutes.

Second eluting isomer (rt=3.36 minutes): rel-(2R,3S,5R)-3-(3-ethyl-4-fluoro-2-methoxyphenyl)-5-methyl-N-(3-methyl-1-(methylsulfonyl)-1H-pyrazol-4-yl)-5-(trifluoromethyl)tetrahydrofuran-2-carboxamide (717, 28.9 mg, 57%) ESI-MS m/z calc. 507.1451, found 508.2 (M+1)⁺; 506.2 (M−1)⁻; Retention time: 3.47 minutes.

### Example 29

rel-(2R,3S,4S,5S)-3-(3,4-difluoro-2-methoxyphenyl)-N-(2-(1,2-dihydroxyethyl)pyridin-4-yl)-4,5-dimethyltetrahydrofuran-2-carboxamide (TFA salt) (718) and rel-(2S,3R,4R,5R)-3-(3,4-difluoro-2-methoxyphenyl)-N-(2-(1,2-dihydroxyethyl)pyridin-4-yl)-4,5-dimethyltetrahydrofuran-2-carboxamide (TFA salt) (719)

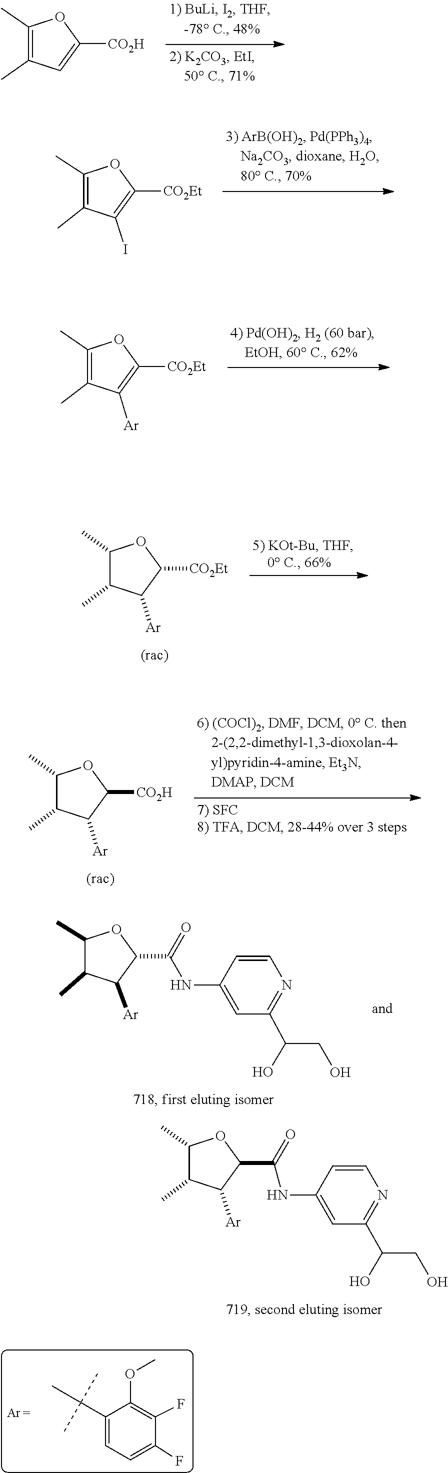

### Step 1:

To a solution of 4,5-dimethylfuran-2-carboxylic acid (1 g, 7.14 mmol) in THF (15 mL) stirring at −78° C. was added BuLi (6.56 mL of 2.5 M, 16.40 mmol) dropwise. The solution was stirred at this temperature for 30 mins before a solution of I₂ (2.35 g, 9.26 mmol) in THF (10 mL) was added. The mixture was warmed to ambient temperature then partitioned between MTBE (30 mL) and water (30 mL). The organic layer was discarded, and the aqueous layer acidified to pH 2 by addition of 1N HCl and the aqueous layer was extracted with MTBE (2×20 mL). The combined organic layers were washed with brine (10 mL), dried (MgSO₄), and concentrated in vacuo to give 3-iodo-4,5-dimethyl-furan-2-carboxylic acid (950 mg, 48%), which was used without further purification. ESI-MS m/z calc. 265.944, found 265.3 (M−1)⁻.

### Step 2:

To a solution of 3-iodo-4,5-dimethyl-furan-2-carboxylic acid (900 mg, 3.38 mmol) in DMF (5 mL) was added K₂CO₃ (1.40 g, 10.13 mmol) and iodoethane (811 μL, 10.14 mmol). The reaction was stirred at 50° C. for 2 hours before being cooled to ambient temperature and partitioned between MTBE (30 mL) and water (30 mL). The aqueous layer was further extracted with MTBE (20 mL) and the combined organic fractions were washed with brine (20 mL), dried (MgSO₄) and concentrated in vacuo. Purification by flash chromatography (12 g SiO₂, 0 to 100% EtOAc in petroleum ether) gave ethyl 3-iodo-4,5-dimethyl-furan-2-carboxylate (800 mg, 71%) as a white solid. ESI-MS m/z calc. 293.97528, found 295.3 (M+1)⁺.

### Step 3:

To a solution of ethyl 3-iodo-4,5-dimethyl-furan-2-carboxylate (700 mg, 2.38 mmol) in dioxane (6 mL) was added (3,4-difluoro-2-methoxy-phenyl)boronic acid (492 mg, 2.62 mmol), Pd(PPh₃)₄ (343 mg, 0.30 mmol), Na₂CO₃ (3.57 mL of 2 M, 7.14 mmol), and water (2 mL). The mixture was heated to 80° C. for 2 hours then cooled to ambient temperature and partitioned between EtOAc (30 mL) and water (30 mL). The aqueous layer was further extracted with EtOAc (50 mL) and combined organic layers were washed with brine (20 mL), dried (MgSO₄), and concentrated in vacuo. Purification by flash chromatography (12 g SiO₂, 0 to 100% EtOAc in petroleum ether) gave ethyl 3-(3,4-difluoro-2-methoxy-phenyl)-4,5-dimethyl-furan-2-carboxylate (520 mg, 70%) as a white solid. ¹H NMR (500 MHz, Chloroform-d) δ 6.97-6.82 (m, 2H), 4.22 (q, J=7.1 Hz, 2H), 3.81 (d, J=2.0 Hz, 3H), 2.37 (d, J=0.8 Hz, 3H), 1.80 (d, J=0.8 Hz, 3H), 1.19 (t, J=7.1 Hz, 3H) ppm. ESI-MS m/z calc. 310.10165, found 311.4 (M+1)⁺.

### Step 4:

A solution of ethyl 3-(3,4-difluoro-2-methoxy-phenyl)-4,5-dimethyl-furan-2-carboxylate (350 mg, 1.128 mmol) in ethanol (2 mL) was circulated through a 70 mm Pd(OH)₂ catalyst cartridge on an H-cube apparatus at 60° C. under 60 bar pressure of hydrogen for 48 hours before being concentrated in vacuo to give ethyl rac-(2S,3S,4S,5S)-3-(3,4-difluoro-2-methoxy-phenyl)-4,5-dimethyl-tetrahydrofuran-2-carboxylate (245 mg, 62%). ¹H NMR (500 MHz, Chloroform-d) δ 7.19-7.08 (m, 1H), 6.72 (td, J=9.3, 7.6 Hz, 1H), 4.55 (d, J=6.1 Hz, 1H), 4.25 (dq, J=9.1, 6.6 Hz, 1H), 4.19-4.02 (m, 1H), 4.02-3.81 (m, 5H), 2.79 (ddt, J=16.4, 8.9, 7.4 Hz, 1H), 1.27-1.04 (m, 3H), 0.86 (t, J=7.1 Hz, 3H), 0.55 (d, J=7.4 Hz, 3H) ppm. ESI-MS m/z calc. 314.13297, found 315.4 (M+1)⁺.

### Step 5:

To a solution of ethyl rac-(2S,3S,4S,5S)-3-(3,4-difluoro-2-methoxy-phenyl)-4,5-dimethyl-tetrahydrofuran-2-carboxylate (400 mg, 1.27 mmol) in THF (5 mL) stirring at 0° C. was added KOt-Bu (428 mg, 3.81 mmol). The reaction was stirred for 30 mins before being diluted in MTBE (5 mL) and quenched by addition of 1M HCl. The aqueous layer was extracted with MTBE (5 mL) and the combined organic layers were dried (MgSO₄), filtered and concentrated in vacuo to give rac-(2R,3S,4S,5S)-3-(3,4-difluoro-2-methoxy-phenyl)-4,5-dimethyl-tetrahydrofuran-2-carboxylic acid (270 mg, 66%) as an oil. ESI-MS m/z calc. 286.10165, found 285.4 (M−1)⁻.

### Step 6:

To an ice cooled solution of rac-(2R,3S,4S,5S)-3-(3,4-difluoro-2-methoxy-phenyl)-4,5-dimethyl-tetrahydrofuran-2-carboxylic acid (100 mg, 0.3095 mmol) in 2-methyltetrahydrofuran (2 mL) was added DMF (3 mg, 0.04104 mmol) as a solution in 2-methyltetrahydrofuran and carefully oxalyl chloride (54 μL, 0.6190 mmol). The reaction was stirred and warmed to ambient temperature over 30 minutes. The reaction mixture was then concentrated in vacuo and the residue dissolved in 2-methyltetrahydrofuran (2 mL). This solution was added to an ice cooled solution of 2-(2,2-dimethyl-1,3-dioxolan-4-yl)pyridin-4-amine (66 mg, 0.3398 mmol, second eluting isomer by SFC) and Et₃N (172 μL, 1.234 mmol) in 2-methyltetrahydrofuran (2 mL). The resulting mixture was stirred and warmed to ambient temperature over 1 hour. The reaction mixture was quenched with water (10 mL) and the layers separated. The aqueous layer was extracted with ethylacetate (2×10 mL) and the combined organics extracts were dried (MgSO₄), filtered, and concentrated in vacuo to afford a mixture of rel-(2R,3S,4S,5S)-3-(3,4-difluoro-2-methoxyphenyl)-N-(2-(1,2-dihydroxyethyl)pyridin-4-yl)-4,5-dimethyltetrahydrofuran-2-carboxamide and rel-(2S,3R,4R,5R)-3-(3,4-difluoro-2-methoxyphenyl)-N-(2-(1,2-dihydroxyethyl)pyridin-4-yl)-4,5-dimethyltetrahydrofuran-2-carboxamide.

### Step 7 and 8:

The mixture obtained from Step 6 was purified by chiral SFC using a Chiralpak ID column, 5 μm particle size, 25 cm×20 mm from Daicel. To each separated isomer in DCM (2 mL) was added TFA (40 μL, 0.5192 mmol) and the mixtures were stirred for 2 hours at ambient temperature. The mixtures were then concentrated and lyophilized to give:

Precursor was first eluting isomer: rel-(2R,3S,4S,5S)-3-(3,4-difluoro-2-methoxyphenyl)-N-(2-(1,2-dihydroxyethyl)pyridin-4-yl)-4,5-dimethyltetrahydrofuran-2-carboxamide (TFA salt) (718, 4 mg, 28%) as a white solid. ¹H NMR (500 MHz, Chloroform-d) δ 9.96 (s, 1H), 8.43 (s, 1H), 8.14 (s, 1H), 8.06 (s, 1H), 7.05 (t, J=7.0 Hz, 1H), 6.89 (q, J=8.6 Hz, 1H), 5.09 (s, 1H), 4.89 (d, J=10.0 Hz, 1H), 4.62-4.49 (m, 1H), 4.10-3.90 (m, 4H), 3.83 (s, 1H), 2.55 (q, J=6.4 Hz, 1H), 1.45-1.17 (m, 6H), 0.65 (d, J=7.2 Hz, 3H) ppm. ESI-MS m/z calc. 422.1653, found 423.5 (M+1)⁺; 421.5 (M−1); Retention time: 2.62 minutes.

Precursor was second eluting isomer: rel-(2S,3R,4R,5R)-3-(3,4-difluoro-2-methoxyphenyl)-N-(2-(1,2-dihydroxyethyl)pyridin-4-yl)-4,5-dimethyltetrahydrofuran-2-carboxamide (TFA salt) (719, 6.5 mg, 44%) as a white solid. ¹H NMR (500 MHz, DMSO-d₆) δ 10.87 (s, 1H), 8.53 (s, 1H), 8.18 (s, 1H), 8.02 (s, 1H), 7.25 (td, J=6.6, 6.1, 2.9 Hz, 1H), 7.17 (td, J=9.4, 7.6 Hz, 1H), 4.95 (d, J=9.4 Hz, 1H), 6.61-6.64 (m, 1H), 4.80 (s, 1H), 4.61-4.41 (m, 1H), 4.06 (dd, J=9.4, 6.4 Hz, 1H), 3.90 (d, J=1.7 Hz, 3H), 3.62 (s, 1H), 2.09-1.82 (m, 2H), 1.47 (q, J=7.5 Hz, 1H), 1.24 (q, J=6.3, 5.8 Hz, 3H), 0.58 (d, J=7.2 Hz, 3H) ppm. ESI-MS m/z calc. 422.1653, found 423.5 (M+1)⁺; 421.5 (M−1)⁻; Retention time: 2.62 minutes.

### Example 30

(2R,3S,4S,5S)-3-(3,4-difluoro-2-methoxyphenyl)-5-isopropyl-4-methyl-N-(pyridin-3-yl)tetrahydrofuran-2-carboxamide (720)

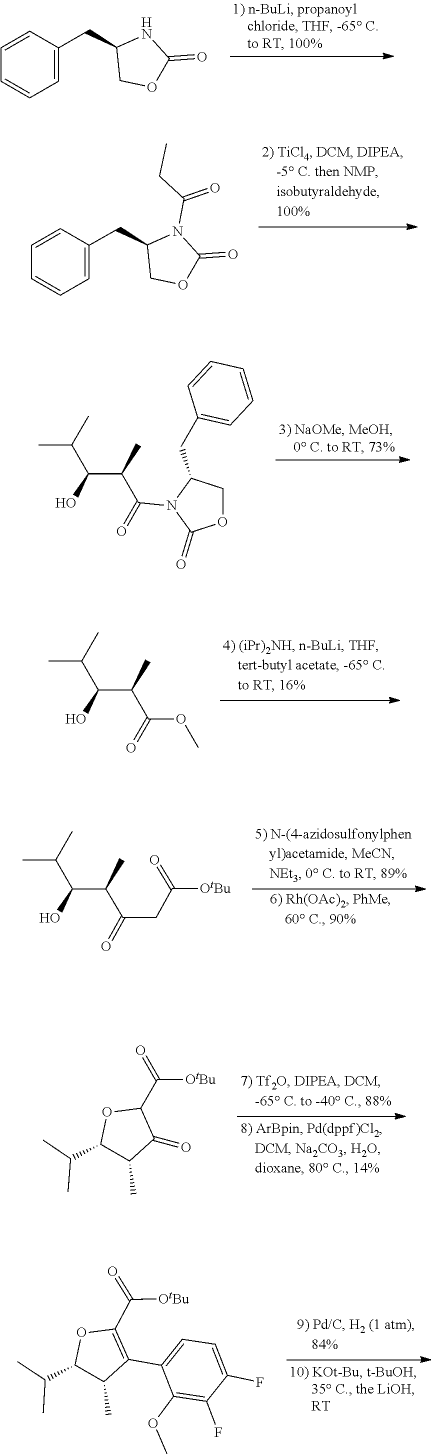

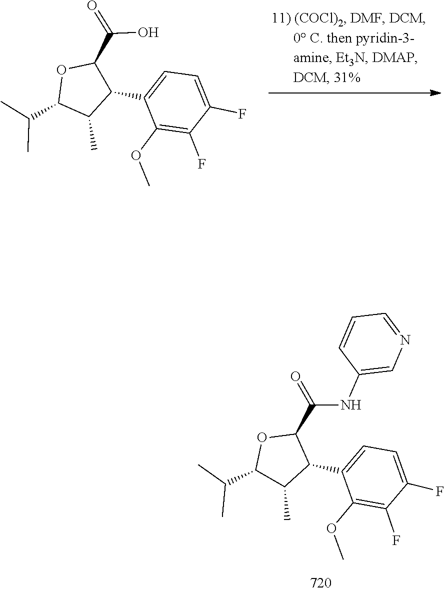 [SMILES: COc1c([C@@H]2[C@H](C)[C@H](C(C)C)O[C@H]2C(=O)O)ccc(F)c1F ; COc1c([C@@H]2[C@H](C)[C@H](C(C)C)O[C@H]2C(=O)Nc2cccnc2)ccc(F)c1F]

### Step 1:

To a solution of (4R)-4-benzyloxazolidin-2-one (29.7 g, 164.26 mmol) in THF (297 mL) cooled to −65° C. was added n-BuLi (65.7 mL of 2.5 M, 164.2 mmol) dropwise, keeping the temperature at −65° C. The resulting mixture was stirred at −65° C. for 30 min before propanoyl chloride (17.059 g, 16.247 mL, 180.69 mmol) was added dropwise. The mixture was stirred at −65° C. for 1 hour and then allowed to warm to ambient temperature overnight. The mixture was quenched by addition of saturated aqueous NH₄Cl solution (300 mL) and the aqueous layer extracted with EtOAc (2×300 mL). The combined organic layers were washed with saturated aqueous NaHCO₃ solution (300 mL) and brine (200 mL), dried (MgSO₄), filtered, and evaporated in vacuo to give (4R)-4-benzyl-3-propanoyl-oxazolidin-2-one (39 g, 100%) as a colorless oil. ¹H NMR (300 MHz, Chloroform-d) δ 7.46-7.14 (m, 5H), 4.69 (ddt, J=9.5, 6.9, 3.4 Hz, 1H), 4.27-4.15 (m, 2H), 3.32 (dd, J=13.4, 3.3 Hz, 1H), 2.98 (qd, J=7.3, 5.9 Hz, 2H), 2.80 (dd, J=13.4, 9.6 Hz, 1H), 1.23 (t, J=7.4 Hz, 3H) ppm. ESI-MS m/z calc. mass 233.105, found, 233.95 [M+1]+.

### Step 2:

To a solution of (4R)-4-benzyl-3-propanoyl-oxazolidin-2-one (13.12 g, 56.25 mmol) in DCM (130 mL) cooled to 0° C. was added titanium tetrachloride (59 mL of 1 M in DCM, 59.00 mmol). The resulting mixture was stirred at 0° C. for 15 mins before DIPEA (8.1620 g, 11 mL, 63.15 mmol) was added and the mixture stirred at this temperature for 40 mins. NMP (5.5512 g, 5.4 mL, 55.999 mmol) was added and the reaction stirred for 10 mins at ambient temperature before isobutyraldehyde (4.2660 g, 5.4 mL, 59.16 mmol) was added and the mixture was stirred at 0° C. for 1 hour and then at ambient temperature overnight. The mixture was quenched with a mixture of water (50 mL) and saturated aqueous NH₄Cl solution (50 mL) and extracted with DCM (3×100 mL). The combined organic extracts were dried (Na₂SO₄), filtered, and concentrated in vacuo. Purification by flash chromatography (SiO₂, 0 to 20% EtOAc to hexane) gave (4R)-4-benzyl-3-[(2R,3S)-3-hydroxy-2,4-dimethyl-pentanoyl]oxazolidin-2-one (16 g, 86%) as a light yellow oil. ¹H NMR (300 MHz, Chloroform-d) δ 7.44-7.16 (m, 5H), 4.72 (ddt, J=9.4, 6.9, 3.3 Hz, 1H), 4.30-4.17 (m, 2H), 3.99 (qd, J=7.0, 2.6 Hz, 1H), 3.56 (dd, J=8.6, 2.6 Hz, 1H), 3.28 (dd, J=13.4, 3.4 Hz, 1H), 2.90 (d, J=3.4 Hz, 1H), 2.81 (dd, J=13.4, 9.4 Hz, 1H), 1.75 (dp, J=8.5, 6.6 Hz, 1H), 1.27 (d, J=7.0 Hz, 3H), 1.06 (d, J=6.6 Hz, 3H), 0.93 (d, J=6.7 Hz, 3H) ppm. ESI-MS m/z calc. 305.1627, found 306.05 (M+1)⁺.

### Step 3:

To a solution of (4R)-4-benzyl-3-[(2R,3S)-3-hydroxy-2,4-dimethyl-pentanoyl]oxazolidin-2-one (100 g, 301.27 mmol) in MeOH (750 mL) stirring at 0° C. was added sodium methoxide (19.609 g, 83 mL of 25% w/w in methanol, 90.74 mmol). The mixture was stirred for 30 mins at ambient temperature before being quenched with saturated aqueous NH₄Cl solution (300 mL) and the aqueous layer extracted with DCM (3×200 mL). The combined organic layers were dried (Na₂SO₄), filtered, and concentrated in vacuo. Purification by flash column chromatography (SiO₂, 10 to 20% diethyl ether in hexane) gave methyl (2R,3S)-3-hydroxy-2,4-dimethyl-pentanoate (40.24 g, 73%) as a colorless liquid, containing 12% hexane by weight. ¹H NMR (300 MHz, Chloroform-d) δ 3.73 (s, 3H), 3.59 (dt, J=7.9, 3.9 Hz, 1H), 2.70 (qd, J=7.2, 3.6 Hz, 1H), 2.45 (dd, J=4.1, 1.8 Hz, 1H), 1.69 (ddd, J=13.3, 8.0, 6.7 Hz, 1H), 1.21 (d, J=7.2 Hz, 3H), 1.03 (d, J=6.6 Hz, 3H), 0.90 (d, J=6.8 Hz, 3H) ppm.

### Step 4:

To a solution of diisopropylamine (67.146 g, 93 mL, 663.56 mmol) in THF (1 L) cooled to -65° C. was added BuLi (228 mL of 2.5 M in hexanes, 570.00 mmol). The mixture was stirred for 30 mins at −65° C. before a solution of tert-butyl acetate (66.143 g, 77 mL, 569.42 mmol) in THF (100 mL) was added dropwise followed by solution of methyl (2R,3S)-3-hydroxy-2,4-dimethyl-pentanoate (40 g, 189.75 mmol) in THF (100 mL). The mixture was stirred for 1 hour at −50° C. and then allowed to warm to ambient temperature overnight. The reaction was quenched by addition of ice-water (800 mL) and extracted with DCM (3×400 mL). The combined organic layers were washed with saturated NaHCO₃ (500 mL), water (2×500 mL), dried (Na₂SO₄), filtered, and evaporated in vacuo. Purification by reverse phase flash chromatography (SiO₂ C18, acetonitrile/water 0 to 60%) gave tert-butyl (4R,5S)-5-hydroxy-4,6-dimethyl-3-oxo-heptanoate (7.95 g, 16%) as a yellow oil. ¹H NMR (300 MHz, Chloroform-d) δ 3.62 (dt, J=8.7, 3.2 Hz, 1H), 3.54-3.39 (m, 2H), 2.88 (qd, J=7.2, 2.8 Hz, 1H), 2.57 (d, J=3.7 Hz, 1H), 1.71 (ddt, J=13.3, 8.6, 6.7 Hz, 1H), 1.49 (s, 9H), 1.17 (d, J=7.2 Hz, 3H), 1.05 (d, J=6.5 Hz, 3H), 0.89 (d, J=6.8 Hz, 3H) ppm.

### Step 5:

To a solution of N-(4-azidosulfonylphenyl)acetamide (10.33 g, 43.00 mmol) in acetonitrile (160 mL) was added tert-butyl (4R,5S)-5-hydroxy-4,6-dimethyl-3-oxo-heptanoate (7.9 g, 30.72 mmol). The mixture was cooled to 0° C. and triethylamine (9.29 g, 12.8 mL, 91.84 mmol) was added. The reaction mixture was warmed to ambient temperature and stirred overnight before being concentrated. Purification by flash chromatography gave tert-butyl (4R,5S)-2-diazo-5-hydroxy-4,6-dimethyl-3-oxo-heptanoate (7.58 g, 89%) as a yellow oil. ¹H NMR (300 MHz, Chloroform-d) δ 3.78 (qd, J=7.1, 2.5 Hz, 1H), 3.54 (dt, J=8.6, 2.7 Hz, 1H), 3.10 (d, J=2.8 Hz, 1H), 1.81-1.66 (m, 1H), 1.55 (s, 9H), 1.16 (d, J=7.1 Hz, 3H), 1.05 (d, J=6.6 Hz, 3H), 0.92 (d, J=6.8 Hz, 3H) ppm. ESI-MS m/z calc. 270.158, found 271.1 (M+1)⁺.

### Step 6:

To a suspension of rhodium (II) acetate (134 mg, 0.30 mmol) in toluene (25 mL) stirring at 60° C. was added a solution of tert-butyl (4R,5S)-2-diazo-5-hydroxy-4,6-dimethyl-3-oxo-heptanoate (8.63 g, 30.33 mmol) in toluene (78 mL). The mixture was stirred at 60° C. for 1 hour, then cooled to ambient temperature, filtered through filter paper and concentrated in vacuo to give tert-butyl (4R,5S)-5-isopropyl-4-methyl-3-oxo-tetrahydrofuran-2-carboxylate (7.34 g, 90%) as a light yellow oil, as a presumed mixture of diastereoisomers (epimeric at ester).

### Step 7:

To a solution of tert-butyl (4R,5S)-5-isopropyl-4-methyl-3-oxo-tetrahydrofuran-2-carboxylate (500 mg, 2.06 mmol) in DCM (15 mL) stirring at a cooled −65° C. was added DIPEA (1.1 mL, 6.32 mmol) and trifluoromethylsulfonyl trifluoromethanesulfonate (0.45 mL, 2.67 mmol). The reaction mixture was stirred for 2 hours at −65° C. before further trifluoromethylsulfonyl trifluoromethanesulfonate (0.45 mL, 2.66 mmol) was added. The mixture was stirred at −60° C. for 1 hour and then further DIPEA (0.4 mL, 2.30 mmol) and trifluoromethylsulfonyl trifluoromethanesulfonate (0.45 mL, 2.66 mmol) were added and the mixture was stirred for 1 hour at −60° C. and 1 hour at −40° C. The mixture was quenched with saturated aqueous NaHCO₃ solution (15 mL) and the aqueous layer extracted with DCM (3×10 mL). The combined organic layers were dried (MgSO₄), filtered, and evaporated in vacuo. The residue was dissolved in EtOAc (30 mL) and washed with 1M HCl (3×30 mL). The organic layer was dried (Na₂SO₄), filtered, and concentrated in vacuo to give tert-butyl (2S,3R)-2-isopropyl-3-methyl-4-(trifluoromethylsulfonyloxy)-2,3-dihydrofuran-5-carboxylate (676 mg, 88%) as a brown oil, which was used without further purification.

### Step 8:

A mixture of crude tert-butyl (2S,3R)-2-isopropyl-3-methyl-4-(trifluoromethylsulfonyloxy)-2,3-dihydrofuran-5-carboxylate (676 mg, 1.81 mmol), 2-(3,4-difluoro-2-methoxy-phenyl)-4,4,5,5-tetramethyl-1,3,2-dioxaborolane (731 mg, 2.71 mmol), and sodium carbonate (478 mg, 4.51 mmol) in dioxane (13.5 mL) and water (3.5 mL) in a pressure glass reactor was degassed by bubbling argon through it for 15 mins. Next Pd(dppf)Cl₂-DCM (206 mg, 0.2523 mmol) was added and the reactor was sealed. The reaction mixture was stirred at 80° C. overnight before being cooled to ambient temperature, diluted with EtOAc (20 mL), filtered through celite and concentrated in vacuo. Purification by flash chromatography gave tert-butyl (2S,3S)-4-(3,4-difluoro-2-methoxy-phenyl)-2-isopropyl-3-methyl-2,3-dihydrofuran-5-carboxylate (107 mg, 14%) as a yellow oil. ¹H NMR (300 MHz, Chloroform-d) δ 6.98-6.74 (m, 2H), 4.14 (dd, J=10.2, 8.0 Hz, 1H), 3.92 (d, J=1.8 Hz, 3H), 3.18-2.99 (m, 1H), 2.14 (dp, J=10.1, 6.4 Hz, 1H), 1.30 (s, 9H), 1.19 (d, J=6.5 Hz, 3H), 0.97 (dd, J=7.8, 6.8 Hz, 6H) ppm; ¹⁹F NMR (376 MHz, Chloroform-d) δ−136.92-−137.21 (m), −155.04-−155.40 (m) ppm.

### Step 9:

Ethanol (3.5 mL) was added to a mixture of tert-butyl (2S,3S)-4-(3,4-difluoro-2-methoxy-phenyl)-2-isopropyl-3-methyl-2,3-dihydrofuran-5-carboxylate (118 mg, 0.32 mmol) and Pd/C (Degussa, wet, 350 mg, 0.33 mmol). The mixture was degassed and stirred under a balloon of hydrogen for 4 days before being filtered through celite and washing with EtOAc. The filtrate was concentrated in vacuo to give tert-butyl (2S,3S,4S,5S)-3-(3,4-difluoro-2-methoxy-phenyl)-5-isopropyl-4-methyl-tetrahydrofuran-2-carboxylate (100 mg, 84%). ¹H NMR (400 MHz, Chloroform-d) δ 7.11-7.03 (m, 1H), 6.83-6.75 (m, 1H), 4.45 (d, J=7.9 Hz, 1H), 4.23 (t, J=8.3 Hz, 1H), 3.93 (d, J=1.5 Hz, 3H), 3.49 (dd, J=9.9, 6.9 Hz, 1H), 2.76-2.63 (m, 1H), 1.97-1.83 (m, 1H), 1.15 (s, 9H), 1.13 (d, J=6.5 Hz, 3H), 0.90 (d, J=6.5 Hz, 3H), 0.70 (d, J=7.4 Hz, 3H) ppm.

### Step 10:

Tert-butyl (2S,3S,4S,5S)-3-(3,4-difluoro-2-methoxy-phenyl)-5-isopropyl-4-methyl-tetrahydrofuran-2-carboxylate (100 mg, 0.27 mmol) and potassium tert-butoxide (60 mg, 0.53 mmol) were mixed in tert-butanol (2.6 mL) and stirred at ambient temperature. After 1 hour the reaction was heated to 35° C. After 2 hours at this temperature the reaction was cooled to ambient temperature, LiOH (400 μL of 2 M, 0.80 mmol) was added and the reaction stirred at ambient temperature for 16 h. The reaction was diluted with EtOAc and quenched with 1 M aqueous HCl. The aqueous layer was separated and extracted with EtOAc. The combined organic layers were dried (MgSO₄), filtered and concentrated in vacuo to afford (2R,3S,4S,5S)-3-(3,4-difluoro-2-methoxy-phenyl)-5-isopropyl-4-methyl-tetrahydrofuran-2-carboxylic acid (100 mg) as a white solid, which was used without further purification.

### Step 11:

To a solution of (2R,3S,4S,5S)-3-(3,4-difluoro-2-methoxy-phenyl)-5-isopropyl-4-methyl-tetrahydrofuran-2-carboxylic acid (84.87 mg, 0.2700 mmol) in DCM (3 mL) was added DMF (5 μL, 0.06457 mmol) and carefully oxalyl chloride (70 μL, 0.8024 mmol) at 0° C. Visible gas evolution. After 45 mins the reaction mixture was concentrated in vacuo, then diluted in DCM (1.5 mL) and added dropwise to a solution of pyridin-3-amine (40 mg, 0.4250 mmol) and Et₃N (200 μL, 1.435 mmol) in DCM (1.5 mL) at 0° C. DMAP (4 mg, 0.03274 mmol) was added and the reaction was warmed to ambient temperature after 10 minutes and stirred for 16 hours. The reaction mixture was diluted with DCM and washed with 1 M HCl solution, dried (MgSO₄), filtered and concentrated in vacuo directly on to silica gel. Purification (24 g SiO₂, 0 to 100% ethylacetate in petroleum ether) lyophilization gave (2R,3S,4S,5S)-3-(3,4-difluoro-2-methoxyphenyl)-5-isopropyl-4-methyl-N-(pyridin-3-yl)tetrahydrofuran-2-carboxamide (720, 33.7 mg, 31%) as a white solid. ¹H NMR (500 MHz, DMSO-d₆) δ 10.01 (s, 1H), 8.75 (d, 1H), 8.27 (dd, J=4.7, 1.5 Hz, 1H), 8.03 (ddd, J=8.4, 2.6, 1.5 Hz, 1H), 7.33 (dd, J=8.3, 4.7 Hz, 1H), 7.24-7.09 (m, 2H), 4.87 (d, J=10.1 Hz, 1H), 4.09 (dd, J=10.1, 5.8 Hz, 1H), 3.90 (d, J=1.7 Hz, 3H), 3.85 (dd, J=10.1, 3.8 Hz, 1H), 2.49-2.45 (m, 1H), 1.77-1.63 (m, 1H), 1.06 (d, J=6.4 Hz, 3H), 0.86 (d, J=6.6 Hz, 3H), 0.58 (d, J=7.1 Hz, 3H) ppm. ESI-MS m/z calc. 390.1755, found 391.6 (M+1)⁺; 389.7 (M−1; Retention time: 3.34 minutes.

### Example 31

rel-(2R,3S,4S)-3-(3,4-difluoro-2-methoxyphenyl)-N-(2-(1,2-dihydroxyethyl)pyridin-4-yl)-4,5,5-trimethyltetrahydrofuran-2-carboxamide (721), rel-(2S,3R,4R)-3-(3,4-difluoro-2-methoxyphenyl)-N-(2-(1,2-dihydroxyethyl)pyridin-4-yl)-4,5,5-trimethyltetrahydrofuran-2-carboxamide, rel-(2S,3R,4S)-3-(3,4-difluoro-2-methoxyphenyl)-N-(2-(1,2-dihydroxyethyl)pyridin-4-yl)-4,5,5-trimethyltetrahydrofuran-2-carboxamide, and rel-(2R,3S,4R)-3-(3,4-difluoro-2-methoxyphenyl)-N-(2-(1,2-dihydroxyethyl)pyridin-4-yl)-4,5,5-trimethyltetrahydrofuran-2-carboxamide (722, 723, 724)

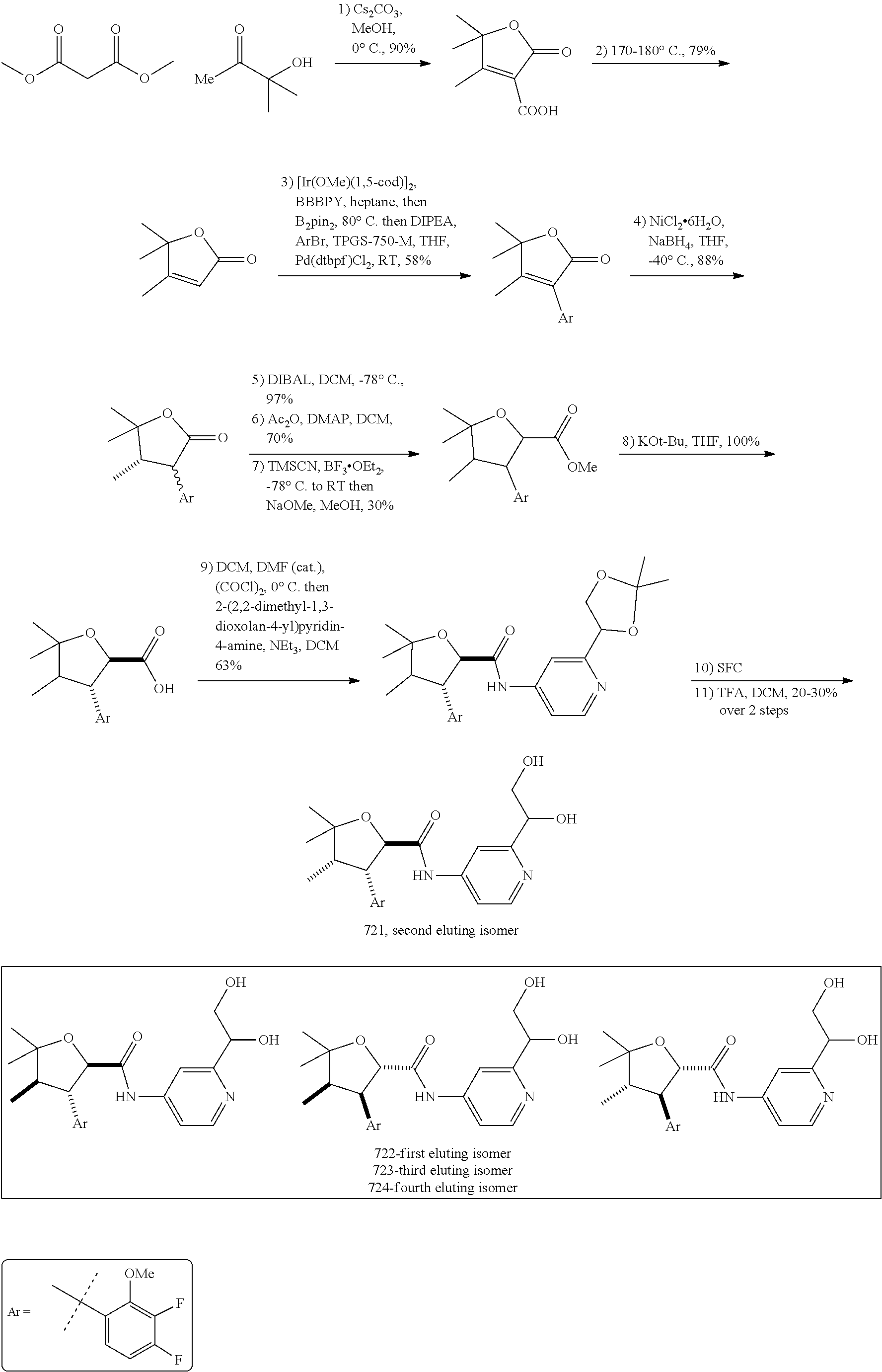

### Step 1:

A solution of 3-hydroxy-3-methyl-butan-2-one (39 g, 381.86 mmol) and dimethyl propanedioate (25 g, 21.74 mL, 189.23 mmol) in MeOH (550 mL) was cooled to 0° C. and stirred under nitrogen. Cs₂CO₃ (127 g, 389.79 mmol) was added and the mixture was stirred overnight. The reaction was then cooled to 0° C. and HCl (630 mL of 1 M, 630.00 mmol) was added. The reaction mixture was concentrated to remove the MeOH, and then EtOAc (800 mL) was added and the layers were separated. The aqueous layer was extracted with EtOAc (2×500 mL) and the combined organic layers dried (Na₂SO₄) and concentrated in vacuo. The residue was triturated with n-pentane to give 4,5,5-trimethyl-2-oxo-furan-3-carboxylic acid (29 g, 90%) as a off-white solid. ¹H NMR (400 MHz, DMSO-d₆) δ 13.13 (s, 1H), 2.28 (s, 3H), 1.42 (s, 6H) ppm.

### Step 2:

4,5,5-trimethyl-2-oxo-furan-3-carboxylic acid (17 g, 99.904 mmol) was heated at 170° C.-180° C. for 4 hours then cooled to ambient temperature. Purification by flash chromatography (SiO₂, 15% EtOAc in hexane) gave 4,5,5-trimethylfuran-2-one (10 g, 79%) as an off-white solid. ¹H NMR (400 MHz, DMSO-d6) δ 5.81 (s, 1H), 2.03 (s, 3H), 1.38 (s, 6H) ppm. ESI-MS m/z calc. 126.0681, found 127.6 (M+1)⁺.

### Step 3:

A mixture of (1,5-cyclooctadine)(methoxy)iridium(I) dimer (1.2 g, 1.81 mmol) and 4,4-di-tert-butyl-2,2′-bipyridine (1.6 g, 5.96 mmol) in n-heptane (50 mL) was degassed and stirred for 15 mins under nitrogen. A solution of 4,5,5-trimethylfuran-2-one (15 g, 118.90 mmol) and bis(pinacolato)diboron (31.8 g, 125.23 mmol) in n-heptane (190 mL) was degassed and stirred under nitrogen for 5 mins and then added to the first solution. The resultant reaction mixture was heated at 80° C. for 2 hours then cooled to ambient temperature. DIPEA (46.75 g, 63 mL, 361.69 mmol) was added to a solution of 1-bromo-3,4-difluoro-2-methoxy-benzene (39.8 g, 178.46 mmol) in TPGS-750-M (40.0 g, 40 mL of 2% w/v, 69.59 mmol) and THF (240 mL) and the mixture was degassed and stirred under nitrogen for 10 mins. This was added to the cooled reaction mixture followed by PdCl₂(dtbpf) (3 g, 4.60 mmol), and the resultant mixture was stirred overnight at ambient temperature. The mixture was diluted with water (200 mL) and extracted with EtOAc (2×700 mL). The combined organic layers were washed with brine (200 mL), dried (Na₂SO₄), and concentrated in vacuo. Purification by flash chromatography (SiO₂, 3 to 5% EtOAc in hexane) gave 3-(3,4-difluoro-2-methoxy-phenyl)-4,5,5-trimethyl-furan-2-one (19 g, 58%) as a white solid. ¹H NMR (400 MHz, DMSO-d₆) δ 7.25-7.23 (m, 1H), 7.10-7.07 (m, 1H), 3.81 (d, J=1.72 Hz, 3H), 1.93 (s, 3H), 1.49 (s, 6H) ppm. ESI-MS m/z calc. 268.0911, found 269.2 (M+1)⁺.

### Step 4:

To a solution of 3-(3,4-difluoro-2-methoxy-phenyl)-4,5,5-trimethyl-furan-2-one (4.2 g, 15.66 mmol) in MeOH (170 mL) and THF (34 mL) stirring at −40° C. was added NiCl₂·6H₂O (3.8 g, 15.99 mmol) and NaBH₄ (3 g, 79.30 mmol). The resulting mixture was stirred for 5 mins before further NiCl₂·6H₂O (3.8 g, 15.99 mmol) and NaBH₄ (3 g, 79.30 mmol) was added. Upon full conversion the reaction was quenched by the addition of saturated aqueous NH₄Cl and the aqueous layer extracted with DCM (2×50 mL). The combined organic extracts were dried (MgSO₄) and concentrated in vacuo to give 3-(3,4-difluoro-2-methoxy-phenyl)-4,5,5-trimethyl-tetrahydrofuran-2-one (3.72 g, 88%) as a 1:1.4 mixture of diasteromers. ESI-MS m/z calc. 270.10675, found 271.4 (M+1).

### Step 5:

To a solution of 3-(3,4-difluoro-2-methoxy-phenyl)-4,5,5-trimethyl-tetrahydrofuran-2-one (3.8 g, 14.06 mmol) in DCM (55 mL) stirring at −78° C. was added DIBAL (17 mL of 1 M, 17.00 mmol). The mixture was stirred at this temperature until complete reaction was observed, then quenched by the addition of saturated ammonium chloride solution (20 mL) and Rochelle's salt (30% w/w solution). The mixture was diluted with DCM (20 mL) and vigorously stirred for 1 h at ambient temperature. The layers were separated and the organic layers were dried (MgSO₄) and concentrated in vacuo to give 3-(3,4-difluoro-2-methoxy-phenyl)-4,5,5-trimethyl-tetrahydrofuran-2-ol (3.70 g, 97%), which was used without further purification.

### Step 6:

To a solution of 3-(3,4-difluoro-2-methoxy-phenyl)-4,5,5-trimethyl-tetrahydrofuran-2-ol (3.7 g, 13.59 mmol) in DCM (40 mL) was added DMAP (850 mg, 6.96 mmol) and acetic anhydride (5.3 mL, 56.17 mmol). The reaction was stirred at ambient temperature overnight then quenched by addition of saturated aqueous sodium bicarbonate solution (50 mL). The mixture was stirred vigorously for 30 mins then the layers were separated. The aqueous layer was extracted with DCM (20 mL) and the combined organic extracts were dried (MgSO₄) and concentrated in vacuo. Purification by flash chromatography (SiO₂) gave [3-(3,4-difluoro-2-methoxy-phenyl)-4,5,5-trimethyl-tetrahydrofuran-2-yl]acetate (3.0 g, 70%) as a mixture of stereoisomers. Data for desired diastereomer: ¹H NMR (500 MHz, Chloroform-d) δ 6.91 (d, J=1.4 Hz, 1H), 6.86-6.76 (m, 2H), 3.91 (d, J=1.7 Hz, 3H), 2.92 (qd, J=7.0, 1.4 Hz, 1H), 2.10 (s, 3H), 1.37 (s, 3H), 1.37 (s, 3H), 1.01 (d, J=7.0 Hz, 3H), 0.91-0.86 (m, 1H) ppm. ESI-MS m/z calc. 314.13297, found 256.6 (M-OAc)⁺.

### Step 7:

To a solution of [3-(3,4-difluoro-2-methoxy-phenyl)-4,5,5-trimethyl-tetrahydrofuran-2-yl]acetate (3 g, 9.544 mmol) in DCM (90 mL) stirring at −78° C. was added trimethylsilyl cyanide (3.3 mL, 24.75 mmol) and diethyloxonio(trifluoro)boranuide (3.7 mL, 29.98 mmol). The mixture was stirred at this temperature for 30 mins then allowed to warm to ambient temperature. Upon completion the mixture was quenched with saturated aqueous sodium bicarbonate solution, the layers separated and the aqueous layer extracted with DCM (3×30 mL). The combined organic layers were dried (Na₂SO₄) and concentrated in vacuo. The residue was dissolved in DCM and filtered through Celite, then concentrated in vacuo. NaOMe (30 mL of 0.5 M in methanol, 15.00 mmol) was added to the residue and the resultant solution stirred at ambient temperature overnight before being quenched by addition of a saturated solution of citric acid. The mixture was stirred at ambient temperature until complete conversion of the amidate was observed, then extracted with DCM (2×30 mL). The combined organic layers were dried (MgSO4) and concentrated in vacuo to give methyl 3-(3,4-difluoro-2-methoxy-phenyl)-4,5,5-trimethyl-tetrahydrofuran-2-carboxylate (900 mg, 30%), which was used in the next step without further purification. ESI-MS m/z calc. 314.13297, found 315.6 (M+1)⁺.

### Step 8:

To a solution of methyl 3-(3,4-difluoro-2-methoxy-phenyl)-4,5,5-trimethyl-tetrahydrofuran-2-carboxylate (440 mg, 1.40 mmol) in THF (5.4 mL) was added KOt-Bu (630 mg, 5.61 mmol) and the mixture stirred at ambient temperature. Upon completion, the reaction was quenched by addition of water and the aqueous layer washed with DCM. The aqueous phase was acidified with 1M HCl and extracted with DCM. The organic layer was evaporated in vacuo to give 3-(3,4-difluoro-2-methoxy-phenyl)-4,5,5-trimethyl-tetrahydrofuran-2-carboxylic acid (420 mg, 100%) in a 1:1.4 ratio of diastereomers. ESI-MS m/z calc. 300.1173, found 299.6 (M−1)⁻.

### Step 9:

To an ice-cooled solution of 3-(3,4-difluoro-2-methoxy-phenyl)-4,5,5-trimethyl-tetrahydrofuran-2-carboxylic acid (105 mg, 0.3497 mmol) in DCM (1.2 mL) was added DMF (3 μL, 0.03874 mmol) and oxalyl chloride (65 μL, 0.7451 mmol) and the mixture stirred and warmed to room temperature over 30 minutes. The reaction mixture was concentrated in vacuo, dissolved in DCM (600 μL) and added to an ice cooled solution of 2-(2,2-dimethyl-1,3-dioxolan-4-yl)pyridin-4-amine (82 mg, 0.4222 mmol, first eluting isomer by SFC) and triethylamine (70 μL, 0.5022 mmol) in DCM (600 μL). The resulting mixture was stirred and warmed to ambient temperature over 2 hours. The reaction was quenched with 1 drop of water and MeOH (2 mL) and concentrated in vacuo. Purification was carried out by flash chromatography to afford -3-(3,4-difluoro-2-methoxy-phenyl)-N-[2-(2,2-dimethyl-1,3-dioxolan-4-yl)-4-pyridyl]-4,5,5-trimethyl-tetrahydrofuran-2-carboxamide (105 mg, 63%) as a mixture of four stereoisomers around the THF ring ((2R,3S,4S), (2S,3R,4R), (2S,3R,4S), (2R,3S,4R)). ESI-MS m/z calc. 476.21228, found 477.6 (M+1)⁺; 475.6 (M−1)⁻; Retention time: 3.41 minutes.

### Step 10:

The mixture of 3-(3,4-difluoro-2-methoxy-phenyl)-N-[2-(2,2-dimethyl-1,3-dioxolan-4-yl)-4-pyridyl]-4,5,5-trimethyl-tetrahydrofuran-2-carboxamide stereoisomers from Step 9 (100 mg, 0.2099 mmol) was purified by chiral SFC using a Chiralpak IC column, 5 μm particle size, 25 cm×20 mm from Daicel, to afford 4 stereoisomers. The separated isomers were subsequently deprotected using a 5:1 mixture of DCM: TFA (2 mL, 25.96 mmol) (5 mL each) at ambient temperature to give:

precursor was first eluting isomer: rel-3-(3,4-difluoro-2-methoxyphenyl)-N-(2-(1,2-dihydroxyethyl)pyridin-4-yl)-4,5,5-trimethyltetrahydrofuran-2-carboxamide (722, 6.4 mg, 26%). ESI-MS m/z calc. 436.18097, found 437.0 (M+1)⁺; 435.1 (M−1)⁻; Retention time: 2.8 minutes.

Precursor was second eluting isomer: rel-(2R,3S,4S)-3-(3,4-difluoro-2-methoxyphenyl)-N-(2-(1,2-dihydroxyethyl)pyridin-4-yl)-4,5,5-trimethyltetrahydrofuran-2-carboxamide (721, 7.7 mg, 31%). ESI-MS m/z calc. 436.18097, found 437.0 (M+1)⁺; 435.1 (M−1)⁻; Retention time: 2.83 minutes.

Precursor was third eluting isomer: rel-3-(3,4-difluoro-2-methoxyphenyl)-N-(2-(1,2-dihydroxyethyl)pyridin-4-yl)-4,5,5-trimethyltetrahydrofuran-2-carboxamide (723, 5.4 mg, 20%). ESI-MS m/z calc. 436.18097, found 437.0 (M+1)⁺; 435.1 (M−1)⁻; Retention time: 2.79 minutes.

Precursor was fourth eluting isomer: rel-3-(3,4-difluoro-2-methoxyphenyl)-N-(2-(1,2-dihydroxyethyl)pyridin-4-yl)-4,5,5-trimethyltetrahydrofuran-2-carboxamide (724, 7.7 mg, 29%). ESI-MS m/z calc. 436.18097, found 437.0 (M+1)⁺; 435.1 (M−1)⁻; Retention time: 2.82 minutes. The following compounds were made using a method similar to Example 31 except that rac-2-(2,2,4-trimethyl-1,3-dioxolan-4-yl)pyridin-4-amine was used in the amide coupling step 9. Chiral SFC separation in step 10 used a (R′R) Whelk-O1 column, followed by an OD-H column to further separated the first eluting peak into 4 isomers and a Lux Cellulose-2 column to further separate the second eluting peak into 2 isomers. A total of 8 stereoisomers were isolated, which were subsequently deprotected in a similar manner.

| | | | | MS | |
|---|---|---|---|---|---|
| Cmpd | | LC/MS | Found | retention | |
| No. | Compound Name | (m/z calc.) | M + 1 | time | SFC details |
| 725 | rel-3-(3,4-difluoro-2- | 450.476 | 451.4 | 2.86 | Precursor was first |
| | methoxyphenyl)-N-(2- | | | | eluting peak by SFC on a |
| | (1,2-dihydroxypropan- | | | | Whelk-01 column; then |
| | 2-yl)pyridin-4-yl)- | | | | first eluting isomer on an |
| | 4,5,5- | | | | OD-H column |
| | trimethyltetrahydrofuran- | | | | |
| | 2-carboxamide | | | | |
| 726 | rel-3-(3,4-difluoro-2- | 450.476 | 451.4 | 2.86 | Precursor was first |
| | methoxyphenyl)-N-(2- | | | | eluting peak by SFC on a |
| | (1,2-dihydroxypropan- | | | | Whelk-01 column; then |
| | 2-yl)pyridin-4-yl)- | | | | second eluting isomer on |
| | 4,5,5- | | | | an OD-H column |
| | trimethyltetrahydrofuran- | | | | |
| | 2-carboxamide | | | | |
| 727 | rel-3-(3,4-difluoro-2- | 450.476 | 451.4 | 2.89 | Precursor was first |
| | methoxyphenyl)-N-(2- | | | | eluting peak by SFC on a |
| | (1,2-dihydroxypropan- | | | | Whelk-01 column; then |
| | 2-yl)pyridin-4-yl)- | | | | third eluting isomer on |
| | 4,5,5- | | | | an OD-H column |
| | trimethyltetrahydrofuran- | | | | |
| | 2-carboxamide | | | | |
| 728 | rel-3-(3,4-difluoro-2- | 450.476 | 451.4 | 2.89 | Precursor was first |
| | methoxyphenyl)-N-(2- | | | | eluting peak by SFC on a |
| | (1,2-dihydroxypropan- | | | | Whelk-01 column; then |
| | 2-yl)pyridin-4-yl)- | | | | fourth eluting isomer on |
| | 4,5,5- | | | | an OD-H column |
| | trimethyltetrahydrofuran- | | | | |
| | 2-carboxamide | | | | |
| 729 | rel-3-(3,4-difluoro-2- | 450.476 | 451.4 | 2.86 | Precursor was second |
| | methoxyphenyl)-N-(2- | | | | eluting peak by SFC on a |
| | (1,2-dihydroxypropan- | | | | Whelk-01 column; then |
| | 2-yl)pyridin-4-yl)- | | | | first eluting isomer on an |
| | 4,5,5- | | | | Lux Cellulose-2 column |
| | trimethyltetrahydrofuran- | | | | |
| | 2-carboxamide | | | | |
| 730 | rel-3-(3,4-difluoro-2- | 450.476 | 451.4 | 2.85 | Precursor was second |
| | methoxyphenyl)-N-(2- | | | | eluting peak by SFC on a |
| | (1,2-dihydroxypropan- | | | | Whelk-01 column; then |
| | 2-yl)pyridin-4-yl)- | | | | second eluting isomer on |
| | 4,5,5- | | | | an Lux Cellulose-2 |
| | trimethyltetrahydrofuran- | | | | column |
| | 2-carboxamide | | | | |
| 731 | rel-(2R,3S,4S)-3-(3,4- | 450.476 | 451.4 | 2.88 | Precursor was third |
| | difluoro-2- | | | | eluting peak by SFC on a |
| | methoxyphenyl)-N-(2- | | | | Whelk-01 column |
| | (1,2-dihydroxypropan- | | | | |
| | 2-yl)pyridin-4-yl)- | | | | |
| | 4,5,5- | | | | |
| | trimethyltetrahydrofuran- | | | | |
| | 2-carboxamide | | | | |
| 732 | rel-(2R,3S,4S)-3-(3,4- | 450.476 | 451.4 | 2.88 | Precursor was fourth |
| | difluoro-2- | | | | eluting peak by SFC on a |
| | methoxyphenyl)-N-(2- | | | | Whelk-01 column |
| | (1,2-dihydroxypropan- | | | | |
| | 2-yl)pyridin-4-yl)- | | | | |
| | 4,5,5- | | | | |
| | trimethyltetrahydrofuran- | | | | |
| | 2-carboxamide | | | | |

### Example 32

### 5-((2R,3S,4S,5R)-3-(3,4-difluoro-5-hydroxy-2-methoxyphenyl)-4,5-dimethyl-5-(trifluoromethyl)tetrahydrofuran-2-carboxamido)picolinamide (733)

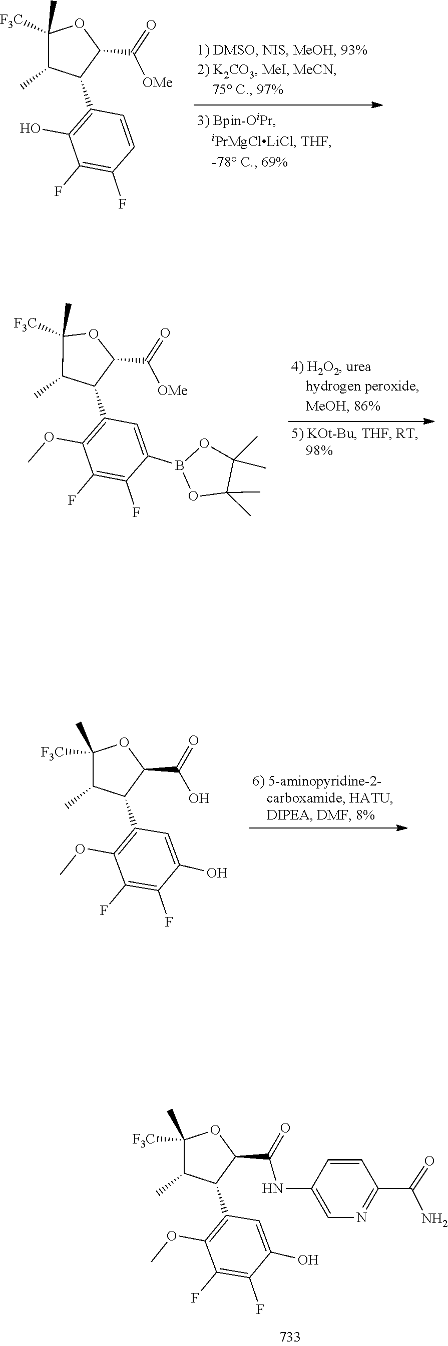

### Step 1:

DMSO (80 μL, 1.127 mmol) and NIS (1.7 g, 7.556 mmol) were sequentially added to a stirred solution of methyl (2S,3S,4S,5R)-3-(3,4-difluoro-2-hydroxyphenyl)-4,5-dimethyl-5-(trifluoromethyl)tetrahydrofuran-2-carboxylate (2.2 g, 6.210 mmol) in MeOH (20 mL) at ambient temperature. The reaction mixture was stirred at ambient temperature under for 30 min. Upon reaction completion, the mixture was concentrated in vacuo. Purification by flash chromatography gave methyl (2S,3S,4S,5R)-3-(3,4-difluoro-2-hydroxy-5-iodophenyl)-4,5-dimethyl-5-(trifluoromethyl)tetrahydrofuran-2-carboxylate (2.78 g, 93%). ¹H NMR (500 MHz, Chloroform-d) δ 7.49 (dt, J=6.4, 2.1 Hz, 1H), 5.56 (d, J=4.9 Hz, 1H), 4.81 (d, J=5.9 Hz, 1H), 4.16 (dd, J=8.3, 5.9 Hz, 1H), 3.60 (s, 3H), 2.75 (p, J=7.7 Hz, 1H), 1.45 (d, J=1.2 Hz, 3H), 0.90-0.85 (m, 3H) ppm. ESI-MS m/z calc. 479.9857, found 481.1 (M+1)⁺; 479.1 (M−1)⁻; Retention time: 0.8 minutes.

### Step 2:

K₂CO₃ (2.5 g, 18.09 mmol) and MeI (1 mL, 16.06 mmol) were successively added to a solution of methyl (2S,3S,4S,5R)-3-(3,4-difluoro-2-hydroxy-5-iodophenyl)-4,5-dimethyl-5-(trifluoromethyl)tetrahydrofuran-2-carboxylate (2.8 g, 5.831 mmol) in MeCN (25 mL). The reaction mixture was heated to 75° C. in a sealed vial for 90 min. Upon reaction completion, the mixture was partitioned between DCM and a saturated aqueous NaCl solution. The organic phase was separated, dried (MgSO₄), filtered, and concentrated in vacuo to give methyl (2S,3S,4S,5R)-3-(3,4-difluoro-5-iodo-2-methoxyphenyl)-4,5-dimethyl-5-(trifluoromethyl)tetrahydrofuran-2-carboxylate (2.8 g, 97%). ¹H NMR (500 MHz, Chloroform-d) δ 7.53 (dq, J=6.5, 1.5 Hz, 1H), 4.80 (d, J=6.1 Hz, 1H), 4.11 (dd, J=8.5, 5.8 Hz, 1H), 3.88 (d, J=2.4 Hz, 3H), 3.56 (s, 3H), 2.73 (p, J=8.4, 7.8 Hz, 1H), 1.45 (d, J=1.1 Hz, 3H), 0.80 (dd, J=7.4, 1.9 Hz, 3H) ppm. ESI-MS m/z calc. 494.00134, found 495.2 (M+1)⁺; Retention time: 1.06 minutes.

### Step 3:

ⁱPrMgCl-LiCl (1.2 mL of 1.3 M in THF, 1.560 mmol) was added dropwise to a stirred solution of methyl (2S,3S,4S,5R)-3-(3,4-difluoro-5-iodo-2-methoxyphenyl)-4,5-dimethyl-5-(trifluoromethyl)tetrahydrofuran-2-carboxylate (700 mg, 1.416 mmol) in THF (6 mL) at −78° C. The resulting solution was stirred for 15 min at −78° C. 2-Isopropoxy-4,4,5,5-tetramethyl-1,3,2-dioxaborolane (350 μL, 1.716 mmol) was added and the reaction mixture was allowed to warm up to ambient temperature. The reaction was quenched by addition of a saturated ammonium chloride solution and extracted with DCM. The organic extracts were dried (MgSO₄), filtered, and concentrated in vacuo. Purification by flash chromatography (12 g SiO₂, 0 to 100% AcOEt in heptane) gave methyl (2S,3S,4S,5R)-3-(3,4-difluoro-2-methoxy-5-(4,4,5,5-tetramethyl-1,3,2-dioxaborolan-2-yl)phenyl)-4,5-dimethyl-5-(trifluoromethyl)tetrahydrofuran-2-carboxylate (480 mg, 69%), which was used in the next step without further purification. ESI-MS m/z calc. 494.1899, found 495.5 (M+1)⁺; Retention time: 1.09 minutes.

### Step 4:

Urea hydrogen peroxide complex (150 mg, 1.595 mmol) was added in one portion to a stirred solution of methyl (2S,3S,4S,5R)-3-(3,4-difluoro-2-methoxy-5-(4,4,5,5-tetramethyl-1,3,2-dioxaborolan-2-yl)phenyl)-4,5-dimethyl-5-(trifluoromethyl)tetrahydrofuran-2-carboxylate (600 mg, 1.214 mmol) in MeOH (2.5 mL). The solution was stirred overnight at ambient temperature. The mixture was concentrated in vacuo. Purification by flash chromatography gave methyl (2S,3S,4S,5R)-3-(3,4-difluoro-5-hydroxy-2-methoxyphenyl)-4,5-dimethyl-5-(trifluoromethyl)tetrahydrofuran-2-carboxylate (400 mg, 86%) as a white solid. ESI-MS m/z calc. 384.0996, found 383.3 (M−1)⁻; Retention time: 0.85 minutes.

### Step 5

Potassium tert-butoxide (940 mg, 8.377 mmol) was added to a stirred solution of methyl (2S,3S,4S,5R)-3-(3,4-difluoro-5-hydroxy-2-methoxyphenyl)-4,5-dimethyl-5-(trifluoromethyl)tetrahydrofuran-2-carboxylate (800 mg, 2.082 mmol) in THF (10 mL) at ambient temperature. Upon reaction completion, the mixture was quenched by addition of a saturated ammonium chloride solution (3 mL) and diluted with DCM (3 mL). The aqueous phase was separated and extracted with DCM (5 mL). The aqueous phase was acidified to pH 0 with 1N HCl. The aqueous extracts were extracted with DCM (2×10 mL). The combined extracts were dried (MgSO₄), filtered and concentrated in vacuo to give (2R,3S,4S,5R)-3-(3,4-difluoro-5-hydroxy-2-methoxyphenyl)-4,5-dimethyl-5-(trifluoromethyl)tetrahydrofuran-2-carboxylic acid (755 mg, 98%), which was used in the next step without further purification. ESI-MS m/z calc. 370.08395, found 371.3 (M+1)⁺; 369.2 (M−1)⁻; Retention time: 0.52 minutes.

### Step 6

HATU (100 mg, 0.2630 mmol) was added to a stirred solution of (2R,3S,4S,5R)-3-(3,4-difluoro-5-hydroxy-2-methoxyphenyl)-4,5-dimethyl-5-(trifluoromethyl)tetrahydrofuran-2-carboxylic acid (50 mg, 0.135 mmol) and DIPEA (80 μL, 0.459 mmol) in DMF (1 mL) at ambient temperature. The reaction mixture was stirred for 10 min at ambient temperature. 5-Aminopyridine-2-carboxamide (30 mg, 0.2188 mmol) was added to the reaction mixture, which was kept under stirring for 2 days. The mixture was diluted with water (10 mL) and extracted with DCM (2×5 mL). The combined organic extracts were dried (MgSO₄) and concentrated in vacuo. Purification by flash chromatography gave 5-((2R,3S,4S,5R)-3-(3,4-difluoro-5-hydroxy-2-methoxyphenyl)-4,5-dimethyl-5-(trifluoromethyl)tetrahydrofuran-2-carboxamido)picolinamide (733, 6.0 mg, 8%). ¹H NMR (500 MHz, Chloroform-d) δ 8.69 (t, J=1.7 Hz, 1H), 8.53 (s, 1H), 8.19 (d, J=1.9 Hz, 2H), 7.72 (s, 1H), 6.81 (dt, J=8.6, 2.5 Hz, 1H), 5.54 (s, 1H), 4.97 (d, J=10.9 Hz, 1H), 4.10 (dd, J=11.0, 8.2 Hz, 1H), 3.90 (d, J=1.7 Hz, 3H), 2.75 (p, J=7.7 Hz, 1H), 1.68 (s, 3H), 0.82 (dt, J=7.4, 2.4 Hz, 3H) ppm; amide NH not observed. ESI-MS m/z calc. 489.13232, found 490.4 (M+1)⁺; 488.3 (M−1)⁻; Retention time: 2.75 minutes.

### Example 33

rel-5-((2R,3S,4S,5R)-3-(6-(difluoromethyl)-2-methoxypyridin-3-yl)-4,5-dimethyl-5-(trifluoromethyl)tetrahydrofuran-2-carboxamido)picolinamide (734) and rel-5-((2S,3R,4R,5S)-3-(6-(difluoromethyl)-2-methoxypyridin-3-yl)-4,5-dimethyl-5-(trifluoromethyl)tetrahydrofuran-2-carboxamido)picolinamide (735)

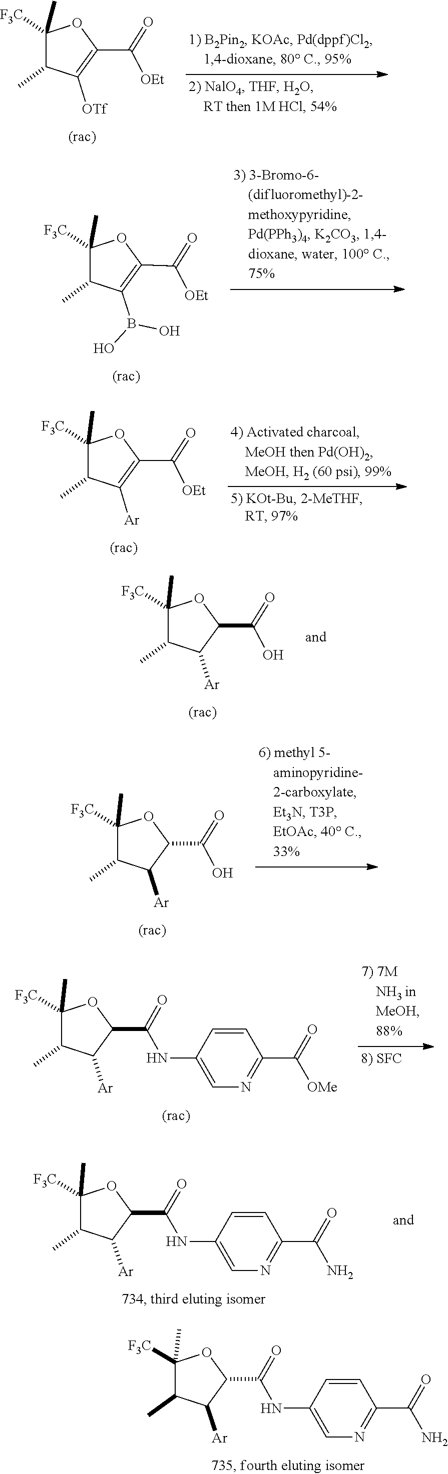

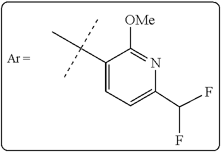

### Step 1:

To a 3 neck 1 L flask, flanked with a thermometer and air condenser, was added ethyl rac-(4R,5R)-4,5-dimethyl-5-(trifluoromethyl)-3-(((trifluoromethyl)sulfonyl)oxy)-4,5-dihydrofuran-2-carboxylate (42 g, 108.7 mmol) and 1,4-dioxane (500 mL). The mixture was stirred, degassed and flushed with nitrogen. KOAc (32 g, 326.1 mmol) was added followed by bis(pinacolato)diboron (32 g, 126.0 mmol). The reaction mixture was evacuated and back filled with nitrogen (×3 cycles). Pd(dppf)Cl₂ (4 g, 5.467 mmol) was added and the mixture was heated to 80° C. for 20 h. The reaction mixture was cooled down to ambient temperature and partitioned between ethyl acetate (300 mL) and water (100 mL). The mixture was filtered through a pad of celite, washing with ethyl acetate (5×100 mL) until no more product came off The filtrate phases were separated. The aqueous layer was extracted with ethyl acetate (2×100 mL). The combined organic layers were passed through a Whatmann phase separation filter paper. The filtrate was concentrated in vacuo to give 47 g of a brown oil. Purification by flash chromatography (Florisil (magnesium silicate), 100% heptane) gave ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (47 g, 95%) as a thick viscous yellow oil. ¹H NMR (500 MHz, Chloroform-d) δ 4.33-4.23 (m, 2H), 3.27-3.18 (m, 1H), 1.55 (d, J=1.1 Hz, 3H), 1.32 (s, 12H), 1.28 (d, J=2.3 Hz, 2H), 1.24 (s, 3H) ppm. ESI-MS m/z calc. 364.1669, found 365.3 (M+1)⁺; Retention time: 1.1 minutes.

### Step 2:

Ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (47 g) was dissolved in a mixture of water (50 mL) and THF (100 mL). Sodium periodate (50 g, 233.8 mmol) was added and the reaction was stirred for 1 h at ambient temperature. The reaction mixture was cooled with an ice bath. 1M HCl (60 mL) was added and reaction mixture was stirred for 1 h. The mixture was diluted with water (50 mL) and ethyl acetate (100 mL). A white solid was filtered and washed with ethyl acetate. The filtrate was washed with sodium thiosulphate (shaken vigorously at every wash to remove traces of iodine) (3×50 ml) followed by a brine solution. The combined organic layers were dried (Na₂SO₄) and concentrated in vacuo to give a cream solid (23 g), which was triturated further with cold heptane to afford rac-((4S,5R)-2-(ethoxycarbonyl)-4,5-dimethyl-5-(trifluoromethyl)-4,5-dihydrofuran-3-yl)boronic acid (16.66 g, 54%) as a white solid. ¹H NMR (500 MHz, Chloroform-d) δ 6.84 (s, 2H), 4.38 (q, J=7.1 Hz, 2H), 3.18 (q, J=7.3 Hz, 1H), 1.51 (d, J 1.2 Hz, 3H), 1.39 (t, J 7.1 Hz, 3H), 1.32 (dq, J 7.2, 2.4 Hz, 3H) ppm. ESI-MS m/z calc. 282.08865, found 281.2 (M−1)⁻; Retention time: 0.75 minutes.

### Step 3:

Pd(PPh₃)₄(82 mg, 0.07096 mmol) and an aqueous solution of K₂CO₃ (3.5 mL of 2 M, 7.000 mmol) were successively added to a solution of rac-((4S,5R)-2-(ethoxycarbonyl)-4,5-dimethyl-5-(trifluoromethyl)-4,5-dihydrofuran-3-yl)boronic acid (1 g, 3.546 mmol) and 3-bromo-6-(difluoromethyl)-2-methoxypyridine (902 mg, 3.789 mmol) in 1,4-dioxane (20 mL). The reaction was heated with stirring at 100° C. for 5 h. A further 30 mg of Pd(PPh₃)₄ was added and the mixture was stirred at reflux for 30 min. The reaction mixture was partitioned between water and ethyl acetate. Aqueous brine was added to help separate the layers. The aqueous phase was separated and extracted twice with EtOAc. The combined organic layers were washed with a brine, dried (MgSO₄), and concentrated in vacuo. Purification by flash chromatography (SiO₂, 0 to 25% EtOAc in heptane) gave ethyl rac-(4S,5R)-3-(6-(difluoromethyl)-2-methoxypyridin-3-yl)-4,5-dimethyl-5-(trifluoromethyl)-4,5-dihydrofuran-2-carboxylate (1.05 g, 75%) as a colourless oil. ¹H NMR (500 MHz, Chloroform-d) δ 7.61 (d, J=7.5 Hz, 1H), 7.22 (d, J=7.4 Hz, 1H), 6.52 (t, J=55.6 Hz, 1H), 4.23-4.07 (m, 2H), 3.96 (s, 3H), 3.63 (q, J=7.4 Hz, 1H), 1.70 (d, J=1.1 Hz, 3H), 1.13 (t, J=7.1 Hz, 3H), 1.06 (dq, J=7.3, 2.2 Hz, 3H) ppm. ESI-MS m/z calc. 395.1156, found 396.3 (M+1)⁺; Retention time: 1.05 minutes.

### Step 4:

A solution of ethyl rac-(4S,5R)-3-(6-(difluoromethyl)-2-methoxypyridin-3-yl)-4,5-dimethyl-5-(trifluoromethyl)-4,5-dihydrofuran-2-carboxylate (670 mg, 1.695 mmol) in MeOH (50 mL) was stirred with activated charcoal for 3 h. The mixture was filtered and added to Pd(OH)₂ (505 mg of 20% w/w, 0.7192 mmol) under nitrogen in a Parr bottle. The bottle was connected to the Parr shaker and agitated under hydrogen (60 psi, 4 bar) at ambient temperature over the weekend. The reaction mixture was filtered through a pad of celite and concentrated in vacuo to give a mixture of ethyl rac-(2S,3S,4S,5R)-3-(6-(difluoromethyl)-2-methoxypyridin-3-yl)-4,5-dimethyl-5-(trifluoromethyl)tetrahydrofuran-2-carboxylate and ethyl rac-(2R,3R,4S,5R)-3-(6-(difluoromethyl)-2-methoxypyridin-3-yl)-4,5-dimethyl-5-(trifluoromethyl)tetrahydrofuran-2-carboxylate (670 mg, 99%) as the 2 major diastereoisomers in a ˜1:0.7 ratio. ESI-MS m/z calc. 397.13126, found 398.2 (M+1)⁺; Retention time: 1.03 and 1.08 minutes.

### Step 5:

Potassium tert-butoxide (398 mg, 3.547 mmol) was added to a solution of a mixture of ethyl rac-(2S,3S,4S,5R)-3-(6-(difluoromethyl)-2-methoxypyridin-3-yl)-4,5-dimethyl-5-(trifluoromethyl)tetrahydrofuran-2-carboxylate and ethyl rac-(2R,3R,4S,5R)-3-(6-(difluoromethyl)-2-methoxypyridin-3-yl)-4,5-dimethyl-5-(trifluoromethyl)tetrahydrofuran-2-carboxylate (700 mg, 1.762 mmol) in 2-MeTHF (20 mL). The reaction mixture was stirred for 30 min at ambient temperature. The reaction was quenched by addition of a 2M HCl solution and partitioned between water and EtOAc. The organic layer was separated and washed with brine, dried (MgSO₄) and concentrated in vacuo to give a mixture of rac-(2R,3S,4S,5R)-3-(6-(difluoromethyl)-2-methoxypyridin-3-yl)-4,5-dimethyl-5-(trifluoromethyl)tetrahydrofuran-2-carboxylic acid and rac-(2S,3R,4S,5R)-3-(6-(difluoromethyl)-2-methoxypyridin-3-yl)-4,5-dimethyl-5-(trifluoromethyl)tetrahydrofuran-2-carboxylic acid (630 mg, 97%) as a yellow oil and as the 2 major diastereoisomers in a ˜1:0.8 ratio. ESI-MS m/z calc. 369.09995, found 368.1 (M−1)⁻; Retention time: 0.56 and 0.58 minutes.

### Step 6:

Triethylamine (225 μL, 1.614 mmol) and T3P (450 μL of 50% w/w, 0.7559 mmol) were successively added to a solution of methyl 5-aminopicolinate (102.5 mg, 0.6737 mmol) and a mixture of rac-(2R,3S,4S,5R)-3-(6-(difluoromethyl)-2-methoxypyridin-3-yl)-4,5-dimethyl-5-(trifluoromethyl)tetrahydrofuran-2-carboxylic acid and rac-(2S,3R,4S,5R)-3-(6-(difluoromethyl)-2-methoxypyridin-3-yl)-4,5-dimethyl-5-(trifluoromethyl)tetrahydrofuran-2-carboxylic acid (200 mg, 0.5416 mmol) in ethyl acetate (4 mL). The reaction mixture was stirred at 40° C. overnight. The mixture was partitioned between water and EtOAc. The aqueous layer was extracted with EtOAc. The combined organic phases were washed with brine, dried (MgSO₄), filtered and concentrated in vacuo. Purification by flash chromatography (SiO₂, 0 to 90% EtOAc in heptane) gave methyl rac-5-((2R,3S,4S,5R)-3-(6-(difluoromethyl)-2-methoxypyridin-3-yl)-4,5-dimethyl-5-(trifluoromethyl)tetrahydrofuran-2-carboxamido)picolinate (89 mg, 33%) as a yellow gum and containing small amounts of other stereoisomers. ¹H NMR (500 MHz, Chloroform-d) δ 8.66 (d, J=2.5 Hz, 1H), 8.57 (s, 1H), 8.37 (dd, J=8.7, 2.5 Hz, 1H), 8.11 (d, J=8.6 Hz, 1H), 7.83 (d, J=7.6 Hz, 1H), 7.28 (s, 1H), 6.52 (t, J=55.6 Hz, 1H), 5.11 (d, J=11.0 Hz, 1H), 4.09-4.05 (m, 1H), 3.99 (s, 3H), 3.98 (s, 3H), 2.93 (p, J=7.7 Hz, 1H), 1.72 (s, 3H), 0.77 (dd, J=7.7, 2.3 Hz, 3H) ppm. ESI-MS m/z calc. 503.14795, found 504.3 (M+1)⁺; 502.2 (M−1)⁻; Retention time: 0.97 minutes.

### Step 7:

Methyl rac-5-((2R,3S,4S,5R)-3-(6-(difluoromethyl)-2-methoxypyridin-3-yl)-4,5-dimethyl-5-(trifluoromethyl)tetrahydrofuran-2-carboxamido)picolinate (40 mg, 0.079 mmol) was dissolved in methanolic ammonia (2 mL of 7 M, 14 mmol) and stirred at ambient temperature overnight. The reaction mixture was concentrated in vacuo to give rac-5-((2R,3S,4S,5R)-3-(6-(difluoromethyl)-2-methoxypyridin-3-yl)-4,5-dimethyl-5-(trifluoromethyl)tetrahydrofuran-2-carboxamido)picolinamide (34 mg, 88%). ESI-MS m/z calc. 488.1483, found 489.3 (M+1)⁺; 487.3 (M−1)⁻; Retention time: 3.22 minutes.

### Step 8:

The enantiomers rac-5-((2R,3S,4S,5R)-3-(6-(difluoromethyl)-2-methoxypyridin-3-yl)-4,5-dimethyl-5-(trifluoromethyl)tetrahydrofuran-2-carboxamido)picolinamide (40 mg, 0.082 mmol) were separated by chiral SFC using a Chiralcel OJ column, 5 m particle size, 25 cm×200 mm from Daicel on a Prep-100 SFC instrument from Waters. The enantiomers of residual rac-5-((2S,3R,4S,5R)-3-(6-(difluoromethyl)-2-methoxypyridin-3-yl)-4,5-dimethyl-5-(trifluoromethyl)tetrahydrofuran-2-carboxamido)picolinamide (first and second eluting isomers) were not collected at this point:

Third Eluting Isomer (rt=1.23 min): rel-5-((2R,3S,4S,5R)-3-(6-(difluoromethyl)-2-methoxypyridin-3-yl)-4,5-dimethyl-5-(trifluoromethyl)tetrahydrofuran-2-carboxamido)picolinamide (Trifluoroacetate salt) (734, 10 mg, 41%). ¹H NMR (500 MHz, DMSO-d₆) δ 10.56 (s, 1H), 8.85 (d, J=2.4 Hz, 1H), 8.21 (dd, J=8.6, 2.5 Hz, 1H), 8.03-7.86 (m, 3H), 7.54-7.48 (m, 1H), 7.31 (d, J=7.6 Hz, 1H), 6.86 (t, J=55.0 Hz, 1H), 5.24 (d, J=9.8 Hz, 1H), 4.24-4.17 (m, 1H), 3.94 (s, 3H), 2.91 (p, J=7.5 Hz, 1H), 1.62 (s, 3H), 0.73-0.68 (m, 3H) ppm. ESI-MS m/z calc. 488.1483, found 489.3 (M+1)⁺; 487.3 (M−1)⁻; Retention time: 3.22 minutes.

Fourth Eluting Isomer (rt=1.68 min): rel-5-((2S,3R,4R,5S)-3-(6-(difluoromethyl)-2-methoxypyridin-3-yl)-4,5-dimethyl-5-(trifluoromethyl)tetrahydrofuran-2-carboxamido)picolinamide (735, 7 mg, 33%). ¹H NMR (500 MHz, DMSO-d₆) δ 10.64 (s, 1H), 8.79 (d, J=2.3 Hz, 1H), 8.19 (dd, J=8.5, 2.5 Hz, 1H), 7.93 (dd, J=18.6, 8.2 Hz, 3H), 7.46 (s, 1H), 7.30 (d, J=7.6 Hz, 1H), 6.86 (t, J=55.0 Hz, 1H), 5.20 (d, J=9.8 Hz, 1H), 4.19 (t, J=8.7 Hz, 1H), 3.94 (s, 3H), 2.90 (p, J=7.4 Hz, 1H), 1.61 (s, 3H), 0.71-0.66 (m, 3H) ppm. ESI-MS m/z calc. 488.1483, found 489.3 (M+1)⁺; 487.2 (M−1)⁻; Retention time: 3.22 minutes.

### LC/MS Methods

The compounds were analysed by LC/MS according to one of the following methods, as shown in Table 1.

TABLE 1

| Method | Description | Mentions |
|---|---|---|
| A | Waters ™ BEH C8 1.7 μm 2.1 × 50 mm, 2 to 98% MeCN in H₂O (10 mM | 653 |
| | Ammonium Formate, pH 9), 45° C., Flow rate 0.6 mL/min over 5.0 min | |
| B | Kinetex ® EVO C18 2.6 μm 2.1 × 50 mm, 2 to 98% MeCN in H₂O (10 mM | 11 |
| | Ammonium Formate, pH 9), 45° C., Flow rate 0.7 mL/min over 4.0 min | |
| C | Kinetex ® EVO C18 2.6 μm 2.1 × 50 mm, 2 to 98% MeCN in H₂O (10 mM | 14 |
| | Ammonium Formate, pH 9), 45° C., Flow rate 1.0 mL/min over 1.5 min | |
| D | Waters ™ CSH C18 1.7 μm 2.1 × 50 mm, 2 to 98% MeCN in H₂O (0.1% | 2 |
| | TFA), 45° C., Flow rate 1.0 mL/min over 1.5 min | |
| E | Waters ™ CSH C18 1.7 μm 2.1 × 50 mm, 2 to 98% MeCN in H₂O (0.1% | 35 |
| | TFA, pH 2), 45° C., Flow rate 0.6 mL/min over 5.0 min | |
| F | Waters ™ BEH C18 2.5 μm 2.1 × 50 mm, 2 to 95% MeCN in H₂O (0.1% | 42 |
| | NH₃), 40° C., Flow rate 0.8 mL/min over 4.6 min | |
| G | Waters ™ CSH C18 1.7 μm 2.1 × 50 mm, 2 to 98% MeCN in H₂O (0.1% | 2 |
| | TFA), 45° C., Flow rate 0.6 mL/min over 4.0 min | |
| H | Waters ™ BEH C18 2.5 μm 2.1 × 50 mm, 2 to 95% MeCN in H₂O (0.1% | 1 |
| | NH₃), 40° C., Flow rate 0.8 mL/min over 1.4 min | |
| I | Waters ™ BEH C8 1.7 μm 2.1 × 50 mm, 20 to 70% MeCN in H₂O (0.1% | 1 |
| | NH₃), 40° C., Flow rate 0.8 mL/min over 4.6 min | |
| J | YMC TRIART C18 3.0 μm 2.1 × 33 mm, 2 to 98% MeCN in H₂O (0.05% | |
| | HCOOH), Flow rate 1.0 mL/min over 3 min | |
| K | Waters ™ XBridge C18 5.0 μm 4.6 × 50 mm, 10 to 90% MeCN in H₂O (10 | |
| | mM Ammonium Acetate), Flow rate 1.2 mL/min over 5 min | |
| L | Restek ™ Ultra C18 3.0 μm 2.1 × 30 mm, 2 to 98% MeCN in H₂O (0.05% | |
| | HCOOH), Flow rate 1.0 mL/min over 3 min | |
| M | YMC TRIART C18 3.0 μm 2.1 × 33 mm, 5 to 95% MeCN in H₂O (0.05% | |
| | HCOOH), Flow rate 1.0 mL/min over 5 min | |
| N | Agilent ZORBAX Extend-C18 5.0 μm 4.6 × 50 mm, 10 to 90% MeCN in | |
| | H₂O (10 mM Ammonium Acetate), Flow rate 1.2 mL/min over 5 min | |
| O | Phenomenex Luna C18 3.0 μm 3.0 × 50 mm, 5 to 95% MeCN in H₂O (0.1% | |
| | HCOOH), 45° C., Flow rate 1.5 mL/min over 2.5 min | |
| P | Agilent ZORBAX Extend-C18 5.0 μm 4.6 × 50 mm, 10 to 90% MeCN in | |
| | H₂O (0.05% HCOOH), Flow rate 1.2 mL/min over 5 min | |
| Q | Waters ™ Acquity UPLC BEH C8 1.7 μm 2.1 × 50 mm, 2 to 98% MeCN in | |
| | H₂O (0.05% HCOOH), 40° C., Flow rate 0.8 mL/min over 3 min | |
| R | Chromolith ® Speed ROD C18 2.0 μm 4.6 × 50 mm, 5 to 95% MeCN in H₂O | |
| | (0.1% TFA) over 12 min | |
| S | Waters ™ Cortex C18 2.7 μm 3.0 × 50 mm, 5 to 95% MeCN in H₂O (0.1% | |
| | TFA), 55° C., Flow rate 1.2 mL/min over 4 min | |
| T | YMC TRIART C18 3.0 μm 2.1 × 33 mm, 2 to 98% MeCN in H₂O (0.05% | |
| | HCOOH), Flow rate 1.0 mL/min over 12 min | |
| U | YMC TRIART C18 3.0 μm 2.1 × 33 mm, 2 to 98% MeCN in H₂O (5 mM | |
| | Ammonium Acetate), Flow rate 1.0 mL/min over 3 min | |
| V | Waters ™ CSH C18 1.7 μm 2.1 × 50 mm, 5 to 95% MeCN in H₂O (0.1% | |
| | TFA), 45° C., Flow rate 1.0 mL/min over 4 min | |
| W | Kinetex ® Polar C18 2.6 μm 3.0 × 50 mm, 5 to 95% MeCN in H₂O (0.1% | |
| | HCOOH), Flow rate 1.2 mL/min over 6.0 min | |
| X | Waters ™ CSH C18 1.7 μm 2.1 × 50 mm, 2 to 95% MeCN in water (0.1% | |
| | Formic Acid), 40° C., Flow rate 0.8 mL/min over 1.4 min | |
| Y | Waters ™ BEH C18 2.5 μm 2.1 × 50 mm, 2 to 20% MeCN in H₂O (0.1% | |
| | NH₃), 40° C., Flow rate 0.8 mL/min over 2 min | |
| Z | Kinetex ® Polar C18 2.6 μm 3.0 × 50 mm, 5 to 95% MeCN in H₂O (0.1% | |
| | HCOOH), Flow rate 1.2 mL/min over 3.0 min | |
| AA | Chromolith ® Speed ROD C18 2.0 μm 4.6 × 50 mm, 5 to 95% MeCN in H₂O | |
| | (0.1% TFA) over 6 min | |
| BB | Waters ™ BEH C18 2.5 μm 2.1 × 50 mm, 2 to 50% MeCN in H₂O (0.1% | |
| | NH₃), 40° C., Flow rate 0.8 mL/min over 2 min | |

TABLE 2
LC/MS Analysis

| | | | | | LCMS |
|---|---|---|---|---|---|
| Cpd | | LCMS | | | Retention |
| No. | IUPAC Name | Method | MW | M + 1 | Time |
| 1 | 5-[[(2R,3S,4S,5R)-3-(3,4-difluoro-2- | E [2], F | 473.393 | 474.15 | 2.95 |
| | methoxy-phenyl)-4,5-dimethyl-5- | | | | |
| | (trifluoromethyl)tetrahydrofuran-2- | | | | |
| | carbonyl]amino]pyridine-2- | | | | |
| | carboxamide | | | | |
| 2 | 5-[[(2R,3S,4S,5R)-3-(3,4-difluoro-2- | A | 489.393 | 490.2 | 3.17 |
| | methoxy-phenyl)-4,5-dimethyl-5- | | | | |
| | (trifluoromethyl)tetrahydrofuran-2- | | | | |
| | carbonyl]amino]-1-oxido-pyridin-1- | | | | |
| | ium-2-carboxamide | | | | |
| 3 | (2R,3S,4S,5R)-3-(3,4-difluoro-2- | A | 504.447 | 505.3 | 3.11 |
| | methoxy-phenyl)-N-[2-(1,2- | | | | |
| | dihydroxy-1-methyl-ethyl)-4-pyridyl]- | | | | |
| | 4,5-dimethyl-5- | | | | |
| | (trifluoromethyl)tetrahydrofuran-2- | | | | |
| | carboxamide | | | | |
| 4 | 3-(2-ethoxy-3,4-difluoro-phenyl)-4,5- | A | 522.485 | 523.5 | 3.52 |
| | dimethyl-N-(2-methylsulfonyl-4- | | | | |
| | pyridyl)-5- | | | | |
| | (trifluoromethyl)tetrahydrofuran-2- | | | | |
| | carboxamide | | | | |
| 5 | (2R,3S,4S,5R)-3-(3,4-difluoro-2- | A | 507.451 | 508.4 | 2.96 |
| | methoxy-phenyl)-N-[5-[(2S)-2,3- | | | | |
| | dihydroxypropyl]-1-methyl-pyrazol-3- | | | | |
| | yl]-4,5-dimethyl-5- | | | | |
| | (trifluoromethyl)tetrahydrofuran-2- | | | | |
| | carboxamide | | | | |
| 6 | 3-(3,4-difluoro-2-methoxy-phenyl)- | A | 492.459 | 493.2 | 3.21 |
| | 4,5-dimethyl-N-(2-methylsulfinyl-4- | | | | |
| | pyridyl)-5- | | | | |
| | (trifluoromethyl)tetrahydrofuran-2- | | | | |
| | carboxamide | | | | |
| 7 | 3-(3,4-difluoro-2-methoxy-phenyl)- | A | 492.459 | 493.2 | 3.2 |
| | 4,5-dimethyl-N-(2-methylsulfinyl-4- | | | | |
| | pyridyl)-5- | | | | |
| | (trifluoromethyl)tetrahydrofuran-2- | | | | |
| | carboxamide | | | | |
| 8 | 3-(3,4-difluoro-2-methoxy-phenyl)- | A | 492.459 | 493.1 | 3.21 |
| | 4,5-dimethyl-N-(2-methylsulfinyl-4- | | | | |
| | pyridyl)-5- | | | | |
| | (trifluoromethyl)tetrahydrofuran-2- | | | | |
| | carboxamide | | | | |
| 9 | 3-(3,4-difluoro-2-methoxy-phenyl)- | A | 492.459 | 493.1 | 3.2 |
| | 4,5-dimethyl-N-(2-methylsulfinyl-4- | | | | |
| | pyridyl)-5- | | | | |
| | (trifluoromethyl)tetrahydrofuran-2- | | | | |
| | carboxamide | | | | |
| 10 | 3-(2-ethoxy-3,4-difluoro-phenyl)-4,5- | A | 521.501 | 522.6 | 3.26 |
| | dimethyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 11 | 3-(2-ethoxy-3,4-difluoro-phenyl)-4,5- | A | 521.501 | 522.5 | 3.26 |
| | dimethyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 12 | N-[2-(N,S-dimethylsulfonimidoyl)-4- | A | 535.527 | 537.5 | 3.37 |
| | pyridyl]-3-(2-ethoxy-3,4-difluoro- | | | | |
| | phenyl)-4,5-dimethyl-5- | | | | |
| | (trifluoromethyl)tetrahydrofuran-2- | | | | |
| | carboxamide | | | | |
| 13 | 3-(3,4-difluoro-2-methoxy-phenyl)-N- | D | 491.427 | 492.9 | 2.81 |
| | [5-fluoro-2-(methylaminomethyl)-4- | | | | |
| | pyridyl]-4,5-dimethyl-5- | | | | |
| | (trifluoromethyl)tetrahydrofuran-2- | | | | |
| | carboxamide | | | | |
| 14 | (2R,3S,4S,5R)-3-(3,4-difluoro-2- | C [2], A | 460.394 | 461.45 | 2.015 |
| | methoxy-phenyl)-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 15 | (2R,3S,4S,5R)-3-(3,4-difluoro-2- | E | 559.569 | 560.1 | 2.97 |
| | methoxy-phenyl)-N-[2-[[(2-methoxy- | | | | |
| | 1,1-dimethyl-ethyl)-methyl- | | | | |
| | amino methyl]-4-pyridyl]-4,5- | | | | |
| | dimethyl-5- | | | | |
| | (trifluoromethyl)tetrahydrofuran-2- | | | | |
| | carboxamide | | | | |
| 16 | 6-[[(2R,3S,4S,5R)-3-(3,4-difluoro-2- | A | 513.417 | 514 | 3.12 |
| | methoxy-phenyl)-4,5-dimethyl-5- | | | | |
| | (trifluoromethyl)tetrahydrofuran-2- | | | | |
| | carbonyl]amino]-[1,2,4]triazolo[1,5- | | | | |
| | alpyridine-2-carboxamide | | | | |
| 17 | (2R,3S,4S,5R)-3-(3,4-difluoro-2- | A | 486.432 | 487.6 | 3.25 |
| | methoxy-phenyl)-N-(7-hydroxy-6,7- | | | | |
| | dihydro-5H-cyclopenta[b]pyridin-3- | | | | |
| | yl)-4,5-dimethyl-5- | | | | |
| | (trifluoromethyl)tetrahydrofuran-2- | | | | |
| | carboxamide | | | | |
| 18 | 3-(3,4-difluoro-2-methoxy-phenyl)-N- | A | 461.383 | 462.6 | 3 |
| | [6-(hydroxymethyl)pyridazin-4-yl]- | | | | |
| | 4,5-dimethyl-5- | | | | |
| | (trifluoromethyl)tetrahydrofuran-2- | | | | |
| | carboxamide | | | | |
| 19 | 5-[[(2R,3S,4S,5R)-3-(3,4-difluoro-2- | A | 474.378 | 475.3 | 2.5 |
| | methoxy-phenyl)-4,5-dimethyl-5- | | | | |
| | (trifluoromethyl)tetrahydrofuran-2- | | | | |
| | carbonyl]amino]pyridine-2-carboxylic | | | | |
| | acid | | | | |
| 20 | (2R,3S,4S,5R)-3-(3,4-difluoro-2- | A | 463.398 | 464.3 | 3.04 |
| | methoxy-phenyl)-N-[5- | | | | |
| | (hydroxymethyl)-2-methyl-pyrazol-3- | | | | |
| | yl]-4,5-dimethyl-5- | | | | |
| | (trifluoromethyl)tetrahydrofuran-2- | | | | |
| | carboxamide | | | | |
| 21 | (2R,3S,4S,5R)-3-(3,4-difluoro-2- | A | 463.398 | 464 | 2.91 |
| | methoxy-phenyl)-N-[2- | | | | |
| | (hydroxymethyl)-3-methyl-imidazol- | | | | |
| | 4-yl]-4,5-dimethyl-5- | | | | |
| | (trifluoromethyl)tetrahydrofuran-2- | | | | |
| | carboxamide | | | | |
| 22 | (2R,3S,4S,5R)-3-(3,4-difluoro-2- | A | 521.453 | 523.4 | 3.26 |
| | methoxy-phenyl)-N-[5-fluoro-2-[2- | | | | |
| | hydroxy-1-(methylamino)ethyl]-4- | | | | |
| | pyridyl]-4,5-dimethyl-5- | | | | |
| | (trifluoromethyl)tetrahydrofuran-2- | | | | |
| | carboxamide | | | | |
| 23 | (2R,3S,4S,5R)-3-(3,4-difluoro-2- | A | 529.5 | 530.3 | 3.2 |
| | methoxy-phenyl)-4,5-dimethyl-N-[2- | | | | |
| | [[[(3R)-tetrahydrofuran-3- | | | | |
| | yl]amino methyl]-4-pyridyl]-5- | | | | |
| | (trifluoromethyl)tetrahydrofuran-2- | | | | |
| | carboxamide | | | | |
| 24 | (2R,3S,4S,5R)-3-(3,4-difluoro-2- | A | 510.402 | 510.9 | 3.42 |
| | methoxy-phenyl)-N-[5-fluoro-2-(2- | | | | |
| | fluoro-1-hydroxy-ethyl)-4-pyridyl]- | | | | |
| | 4,5-dimethyl-5- | | | | |
| | (trifluoromethyl)tetrahydrofuran-2- | | | | |
| | carboxamide | | | | |
| 25 | 3-(3,4-difluoro-2-methoxy-phenyl)- | B | 431.357 | 432.7 | 3.15 |
| | 4,5-dimethyl-N-pyridazin-4-yl-5- | | | | |
| | (trifluoromethyl)tetrahydrofuran-2- | | | | |
| | carboxamide | | | | |
| 26 | 3-(3,4-difluoro-2-methoxy-phenyl)- | B | 431.357 | 432.7 | 3.15 |
| | 4,5-dimethyl-N-pyridazin-4-yl-5- | | | | |
| | (trifluoromethyl)tetrahydrofuran-2- | | | | |
| | carboxamide | | | | |
| 27 | 3-(3,4-difluoro-2-methoxy-phenyl)- | A | 460.394 | 461.2 | 3.09 |
| | 4,5-dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 28 | 3-(3,4-difluoro-2-methoxy-phenyl)- | A | 460.394 | 461.1 | 3.09 |
| | 4,5-dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 29 | 3-(3,4-difluoro-2-methoxy-phenyl)- | A | 430.368 | 431.2 | 3.24 |
| | 4,5-dimethyl-N-(3-pyridyl)-5- | | | | |
| | (trifluoromethyl)tetrahydrofuran-2- | | | | |
| | carboxamide | | | | |
| 30 | 3-(3,4-difluoro-2-methoxy-phenyl)- | A | 430.368 | 431.2 | 3.24 |
| | 4,5-dimethyl-N-(3-pyridyl)-5- | | | | |
| | (trifluoromethyl)tetrahydrofuran-2- | | | | |
| | carboxamide | | | | |
| 31 | 3-(3,4-difluoro-2-methoxy-phenyl)- | B | 471.381 | 472.1 | 3.72 |
| | 4,5-dimethyl-N-(tetrazolo[1,5- | | | | |
| | alpyridin-7-yl)-5- | | | | |
| | (trifluoromethyl)tetrahydrofuran-2- | | | | |
| | carboxamide | | | | |
| 32 | 3-(3,4-difluoro-2-methoxy-phenyl)- | B | 471.381 | 472.1 | 3.29 |
| | 4,5-dimethyl-N-(tetrazolo[1,5- | | | | |
| | alpyridin-7-yl)-5- | | | | |
| | (trifluoromethyl)tetrahydrofuran-2- | | | | |
| | carboxamide | | | | |
| 33 | 3-(3,4-difluoro-2-methoxy-phenyl)-N- | A | 505.435 | 506 | 3.35 |
| | [6-(2-methoxyethoxy)pyridazin-4-yl]- | | | | |
| | 4,5-dimethyl-5- | | | | |
| | (trifluoromethyl)tetrahydrofuran-2- | | | | |
| | carboxamide | | | | |
| 34 | 3-(3,4-difluoro-2-methoxy-phenyl)-N- | A | 505.435 | 506 | 3.35 |
| | [6-(2-methoxyethoxy)pyridazin-4-yl]- | | | | |
| | 4,5-dimethyl-5- | | | | |
| | (trifluoromethyl)tetrahydrofuran-2- | | | | |
| | carboxamide | | | | |
| 35 | 3-(3,4-difluoro-2-methoxy-phenyl)- | B | 431.357 | 432.5 | 3.34 |
| | 4,5-dimethyl-N-pyrimidin-4-yl-5- | | | | |
| | (trifluoromethyl)tetrahydrofuran-2- | | | | |
| | carboxamide | | | | |
| 36 | 3-(3,4-difluoro-2-methoxy-phenyl)- | B | 431.357 | 432.6 | 3.34 |
| | 4,5-dimethyl-N-pyrimidin-4-yl-5- | | | | |
| | (trifluoromethyl)tetrahydrofuran-2- | | | | |
| | carboxamide | | | | |
| 37 | 3-(3,4-difluoro-2-methoxy-phenyl)- | B | 431.357 | 432.2 | 3.17 |
| | 4,5-dimethyl-N-pyrimidin-5-yl-5- | | | | |
| | (trifluoromethyl)tetrahydrofuran-2- | | | | |
| | carboxamide | | | | |
| 38 | 3-(3,4-difluoro-2-methoxy-phenyl)- | B | 431.357 | 432.2 | 3.17 |
| | 4,5-dimethyl-N-pyrimidin-5-yl-5- | | | | |
| | (trifluoromethyl)tetrahydrofuran-2- | | | | |
| | carboxamide | | | | |
| 39 | 3-(3,4-difluoro-2-methoxy-phenyl)-N- | A | 505.415 | 506.1 | 3.11 |
| | (4-dimethylphosphorylphenyl)-4,5- | | | | |
| | dimethyl-5- | | | | |
| | (trifluoromethyl)tetrahydrofuran-2- | | | | |
| | carboxamide | | | | |
| 40 | 3-(3,4-difluoro-2-methoxy-phenyl)-N- | A | 505.415 | 506.1 | 3.11 |
| | (4-dimethylphosphorylphenyl)-4,5- | | | | |
| | dimethyl-5- | | | | |
| | (trifluoromethyl)tetrahydrofuran-2- | | | | |
| | carboxamide | | | | |
| 41 | 3-(3,4-difluoro-2-methoxy-phenyl)-N- | A | 505.415 | 506.1 | 3.1 |
| | (3-dimethylphosphorylphenyl)-4,5- | | | | |
| | dimethyl-5- | | | | |
| | (trifluoromethyl)tetrahydrofuran-2- | | | | |
| | carboxamide | | | | |
| 42 | 3-(3,4-difluoro-2-methoxy-phenyl)-N- | A | 505.415 | 506.1 | 3.1 |
| | (3-dimethylphosphorylphenyl)-4,5- | | | | |
| | dimethyl-5- | | | | |
| | (trifluoromethyl)tetrahydrofuran-2- | | | | |
| | carboxamide | | | | |
| 43 | 3-(3,4-difluoro-2-methoxy-phenyl)-N- | A | 490.42 | 491.7 | 3 |
| | [2-(1,2-dihydroxyethyl)-4-pyridyl]- | | | | |
| | 4,5-dimethyl-5- | | | | |
| | (trifluoromethyl)tetrahydrofuran-2- | | | | |
| | carboxamide | | | | |
| 44 | 3-(3,4-difluoro-2-methoxy-phenyl)-N- | A [2], A | 490.42 | 491.6 | 3.005 |
| | [2-(1,2-dihydroxyethyl)-4-pyridyl]- | | | | |
| | 4,5-dimethyl-5- | | | | |
| | (trifluoromethyl)tetrahydrofuran-2- | | | | |
| | carboxamide | | | | |
| 45 | 3-(3,4-difluoro-2-methoxy-phenyl)-N- | A [2], A | 490.42 | 491.65 | 2.995 |
| | [2-(1,2-dihydroxyethyl)-4-pyridyl]- | | | | |
| | 4,5-dimethyl-5- | | | | |
| | (trifluoromethyl)tetrahydrofuran-2- | | | | |
| | carboxamide | | | | |
| 46 | 3-(3,4-difluoro-2-methoxy-phenyl)-N- | A [3], C | 490.42 | 491.633 | 3.007 |
| | [2-(1,2-dihydroxyethyl)-4-pyridyl]- | [2], A | | | |
| | 4,5-dimethyl-5- | | | | |
| | (trifluoromethyl)tetrahydrofuran-2- | | | | |
| | carboxamide | | | | |
| 47 | 3-(3,4-difluoro-2-methoxy-phenyl)-N- | A | 488.448 | | 3.35 |
| | [2-(1-hydroxy-1-methyl-ethyl)-4- | | | | |
| | pyridyl]-4,5-dimethyl-5- | | | | |
| | (trifluoromethyl)tetrahydrofuran-2- | | | | |
| | carboxamide | | | | |
| 48 | 3-(3,4-difluoro-2-methoxy-phenyl)-N- | A | 488.448 | 489.7 | 3.35 |
| | [2-(1-hydroxy-1-methyl-ethyl)-4- | | | | |
| | pyridyl]-4,5-dimethyl-5- | | | | |
| | (trifluoromethyl)tetrahydrofuran-2- | | | | |
| | carboxamide | | | | |
| 49 | 3-(3,4-difluoro-2-methoxy-phenyl)- | A | 508.459 | 509.1 | 3.38 |
| | 4,5-dimethyl-N-(2-methylsulfonyl-4- | | | | |
| | pyridyl)-5- | | | | |
| | (trifluoromethyl)tetrahydrofuran-2- | | | | |
| | carboxamide | | | | |
| 50 | 3-(3,4-difluoro-2-methoxy-phenyl)- | A | 508.459 | 509.1 | 3.38 |
| | 4,5-dimethyl-N-(2-methylsulfonyl-4- | | | | |
| | pyridyl)-5- | | | | |
| | (trifluoromethyl)tetrahydrofuran-2- | | | | |
| | carboxamide | | | | |
| 51 | 3-(3,4-difluoro-2-methoxy-phenyl)-N- | C | 522.485 | 523 | 3.44 |
| | (2-ethylsulfonyl-4-pyridyl)-4,5- | | | | |
| | dimethyl-5- | | | | |
| | (trifluoromethyl)tetrahydrofuran-2- | | | | |
| | carboxamide | | | | |
| 52 | 3-(3,4-difluoro-2-methoxy-phenyl)-N- | C | 522.485 | 523 | 3.44 |
| | (2-ethylsulfonyl-4-pyridyl)-4,5- | | | | |
| | dimethyl-5- | | | | |
| | (trifluoromethyl)tetrahydrofuran-2- | | | | |
| | carboxamide | | | | |
| 53 | 3-(3,4-difluoro-2-methoxy-phenyl)- | A | 507.474 | 508.6 | 3.07 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 54 | 3-(3,4-difluoro-2-methoxy-phenyl)- | A | 507.474 | 508.6 | 3.07 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 55 | 3-(3,4-difluoro-2-methoxy-phenyl)- | A | 507.474 | 508.6 | 3.07 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 56 | 3-(3,4-difluoro-2-methoxy-phenyl)- | A | 507.474 | 508.6 | 3.07 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 57 | (2R,3S,4S,5R)-3-[3,4-difluoro-2-(2- | C | 610.594 | 611.3 | 3.3 |
| | morpholinoethoxy)phenyl]-4,5- | | | | |
| | dimethyl-N-(3-methyl-1- | | | | |
| | methylsulfonyl-pyrazol-4-yl)-5- | | | | |
| | (trifluoromethyl)tetrahydrofuran-2- | | | | |
| | carboxamide | | | | |
| 58 | 3-(3,4-difluoro-2-methoxy-phenyl)-N- | E | 491.427 | 492.2 | 2.83 |
| | [5-fluoro-2-(methylaminomethyl)-4- | | | | |
| | pyridyl]-4,5-dimethyl-5- | | | | |
| | (trifluoromethyl)tetrahydrofuran-2- | | | | |
| | carboxamide | | | | |
| 59 | 3-(3,4-difluoro-2-methoxy-phenyl)-N- | A | 517.489 | | 3.26 |
| | [2-[(2-methoxyethylamino)methyl]-4- | | | | |
| | pyridyl]-4,5-dimethyl-5- | | | | |
| | (trifluoromethyl)tetrahydrofuran-2- | | | | |
| | carboxamide | | | | |
| 60 | 3-(3,4-difluoro-2-methoxy-phenyl)-N- | A | 517.489 | 518.6 | 3.28 |
| | [2-[(2-methoxyethylamino)methyl]-4- | | | | |
| | pyridyl]-4,5-dimethyl-5- | | | | |
| | (trifluoromethyl)tetrahydrofuran-2- | | | | |
| | carboxamide | | | | |
| 61 | 3-(3,4-difluoro-2-methoxy-phenyl)-N- | C [2], A | 460.394 | 461.7 | 3.13 |
| | [2-(hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 62 | 3-(3,4-difluoro-2-methoxy-phenyl)-N- | B | 538.485 | 539.1 | 3.19 |
| | [2-(2-hydroxyethylsulfonyl)-4- | | | | |
| | pyridyl]-4,5-dimethyl-5- | | | | |
| | (trifluoromethyl)tetrahydrofuran-2- | | | | |
| | carboxamide | | | | |
| 63 | 3-[2-(difluoromethoxy)-3,4-difluoro- | A | 467.337 | 468.2 | 3.17 |
| | phenyl]-4,5-dimethyl-N-pyridazin-4- | | | | |
| | yl-5-(trifluoromethyl)tetrahydrofuran- | | | | |
| | 2-carboxamide | | | | |
| 64 | 3-[2-(difluoromethoxy)-3,4-difluoro- | A | 467.337 | 468.2 | 3.17 |
| | phenyl]-4,5-dimethyl-N-pyridazin-4- | | | | |
| | yl-5-(trifluoromethyl)tetrahydrofuran- | | | | |
| | 2-carboxamide | | | | |
| 65 | 3-[2-(difluoromethoxy)-3,4-difluoro- | A | 466.349 | 467.4 | 3.29 |
| | phenyl]-4,5-dimethyl-N-(3-pyridyl)-5- | | | | |
| | (trifluoromethyl)tetrahydrofuran-2- | | | | |
| | carboxamide | | | | |
| 66 | 3-[2-(difluoromethoxy)-3,4-difluoro- | A | 466.349 | 467.4 | 3.29 |
| | phenyl]-4,5-dimethyl-N-(3-pyridyl)-5- | | | | |
| | (trifluoromethyl)tetrahydrofuran-2- | | | | |
| | carboxamide | | | | |
| 67 | 3-[2-(difluoromethoxy)-3,4-difluoro- | A | 496.375 | 497.1 | 3.08 |
| | phenyl]-4,5-dimethyl-N-(1-methyl-6- | | | | |
| | oxo-3-pyridyl)-5- | | | | |
| | (trifluoromethyl)tetrahydrofuran-2- | | | | |
| | carboxamide | | | | |
| 68 | 3-[2-(difluoromethoxy)-3,4-difluoro- | A | 496.375 | 497.1 | 3.08 |
| | phenyl]-4,5-dimethyl-N-(1-methyl-6- | | | | |
| | oxo-3-pyridyl)-5- | | | | |
| | (trifluoromethyl)tetrahydrofuran-2- | | | | |
| | carboxamide | | | | |
| 69 | N-(5-cyano-3-pyridyl)-3-[2- | A | 491.359 | 492.1 | 3.44 |
| | (difluoromethoxy)-3,4-difluoro- | | | | |
| | phenyl]-4,5-dimethyl-5- | | | | |
| | (trifluoromethyl)tetrahydrofuran-2- | | | | |
| | carboxamide | | | | |
| 70 | N-(5-cyano-3-pyridyl)-3-[2- | A | 491.359 | 492.1 | 3.44 |
| | (difluoromethoxy)-3,4-difluoro- | | | | |
| | phenyl]-4,5-dimethyl-5- | | | | |
| | (trifluoromethyl)tetrahydrofuran-2- | | | | |
| | carboxamide | | | | |
| 71 | 3-[2-(difluoromethoxy)-3,4-difluoro- | A | 544.44 | 545.1 | 3.41 |
| | phenyl]-4,5-dimethyl-N-(4- | | | | |
| | methylsulfonyl-2-pyridyl)-5- | | | | |
| | (trifluoromethyl)tetrahydrofuran-2- | | | | |
| | carboxamide | | | | |
| 72 | 3-[2-(difluoromethoxy)-3,4-difluoro- | A | 544.44 | 545.1 | 3.42 |
| | phenyl]-4,5-dimethyl-N-(4- | | | | |
| | methylsulfonyl-2-pyridyl)-5- | | | | |
| | (trifluoromethyl)tetrahydrofuran-2- | | | | |
| | carboxamide | | | | |
| 73 | N-(4-carbamoylphenyl)-3-[2- | A | 508.386 | 509.1 | 3.21 |
| | (difluoromethoxy)-3,4-difluoro- | | | | |
| | phenyl]-4,5-dimethyl-5- | | | | |
| | (trifluoromethyl)tetrahydrofuran-2- | | | | |
| | carboxamide | | | | |
| 74 | N-(4-carbamoylphenyl)-3-[2- | A | 508.386 | 509.1 | 3.22 |
| | (difluoromethoxy)-3,4-difluoro- | | | | |
| | phenyl]-4,5-dimethyl-5- | | | | |
| | (trifluoromethyl)tetrahydrofuran-2- | | | | |
| | carboxamide | | | | |
| 75 | 3-[2-(difluoromethoxy)-3,4-difluoro- | A | 513.406 | 514.1 | 3.09 |
| | phenyl]-N-[1-(2-hydroxyethyl)-3- | | | | |
| | methyl-pyrazol-4-yl]-4,5-dimethyl-5- | | | | |
| | (trifluoromethyl)tetrahydrofuran-2- | | | | |
| | carboxamide | | | | |
| 76 | 3-[2-(difluoromethoxy)-3,4-difluoro- | A | 513.406 | 514.1 | 3.09 |
| | phenyl]-N-[1-(2-hydroxyethyl)-3- | | | | |
| | methyl-pyrazol-4-yl]-4,5-dimethyl-5- | | | | |
| | (trifluoromethyl)tetrahydrofuran-2- | | | | |
| | carboxamide | | | | |
| 77 | 3-[2-(difluoromethoxy)-3,4-difluoro- | A | 496.375 | 497.1 | 3.1 |
| | phenyl]-4,5-dimethyl-N-(1-methyl-2- | | | | |
| | oxo-4-pyridyl)-5- | | | | |
| | (trifluoromethyl)tetrahydrofuran-2- | | | | |
| | carboxamide | | | | |
| 78 | 3-[2-(difluoromethoxy)-3,4-difluoro- | A | 496.375 | 497.1 | 3.1 |
| | phenyl]-4,5-dimethyl-N-(1-methyl-2- | | | | |
| | oxo-4-pyridyl)-5- | | | | |
| | (trifluoromethyl)tetrahydrofuran-2- | | | | |
| | carboxamide | | | | |
| 79 | 3-[2-(difluoromethoxy)-3,4-difluoro- | A | 507.362 | 508 | 3.32 |
| | phenyl]-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 80 | 3-[2-(difluoromethoxy)-3,4-difluoro- | A | 507.362 | 508 | 3.31 |
| | phenyl]-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 81 | 3-[2-(difluoromethoxy)-3,4-difluoro- | A | 526.401 | 527.5 | 3.08 |
| | phenyl]-N-[2-(1,2-dihydroxyethyl)-4- | | | | |
| | pyridyl]-4,5-dimethyl-5- | | | | |
| | (trifluoromethyl)tetrahydrofuran-2- | | | | |
| | carboxamide | | | | |
| 82 | 3-[2-(difluoromethoxy)-3,4-difluoro- | A | 526.401 | 527.5 | 3.08 |
| | phenyl]-N-[2-(1,2-dihydroxyethyl)-4- | | | | |
| | pyridyl]-4,5-dimethyl-5- | | | | |
| | (trifluoromethyl)tetrahydrofuran-2- | | | | |
| | carboxamide | | | | |
| 83 | 3-[2-(difluoromethoxy)-3,4-difluoro- | A | 524.429 | 525.7 | 3.4 |
| | phenyl]-N-[2-(1-hydroxy-1-methyl- | | | | |
| | ethyl)-4-pyridyl]-4,5-dimethyl-5- | | | | |
| | (trifluoromethyl)tetrahydrofuran-2- | | | | |
| | carboxamide | | | | |
| 84 | 3-[2-(difluoromethoxy)-3,4-difluoro- | A | 524.429 | 525.7 | 3.4 |
| | phenyl]-N-[2-(1-hydroxy-1-methyl- | | | | |
| | ethyl)-4-pyridyl]-4,5-dimethyl-5- | | | | |
| | (trifluoromethyl)tetrahydrofuran-2- | | | | |
| | carboxamide | | | | |
| 85 | 3-[2-(difluoromethoxy)-3,4-difluoro- | A [2], A | 544.44 | 545.05 | 3.36 |
| | phenyl]-4,5-dimethyl-N-(2- | | | | |
| | methylsulfonyl-4-pyridyl)-5- | | | | |
| | (trifluoromethyl)tetrahydrofuran-2- | | | | |
| | carboxamide | | | | |
| 86 | 3-[2-(difluoromethoxy)-3,4-difluoro- | A [2], A | 544.44 | 545.3 | 3.38 |
| | phenyl]-4,5-dimethyl-N-(2- | | | | |
| | methylsulfonyl-4-pyridyl)-5- | | | | |
| | (trifluoromethyl)tetrahydrofuran-2- | | | | |
| | carboxamide | | | | |
| 87 | 3-[2-(difluoromethoxy)-3,4-difluoro- | A | 560.439 | 561.1 | 3.08 |
| | phenyl]-4,5-dimethyl-N-(5- | | | | |
| | methylsulfonyl-1-oxido-pyridin-1- | | | | |
| | ium-3-yl)-5- | | | | |
| | (trifluoromethyl)tetrahydrofuran-2- | | | | |
| | carboxamide | | | | |
| 88 | 3-[2-(difluoromethoxy)-3,4-difluoro- | A | 560.439 | 561 | 3.07 |
| | phenyl]-4,5-dimethyl-N-(5- | | | | |
| | methylsulfonyl-1-oxido-pyridin-1- | | | | |
| | ium-3-yl)-5- | | | | |
| | (trifluoromethyl)tetrahydrofuran-2- | | | | |
| | carboxamide | | | | |
| 89 | 3-[2-(difluoromethoxy)-3,4-difluoro- | A | 558.466 | 559 | 3.45 |
| | phenyl]-N-(2-ethylsulfonyl-4- | | | | |
| | pyridyl)-4,5-dimethyl-5- | | | | |
| | (trifluoromethyl)tetrahydrofuran-2- | | | | |
| | carboxamide | | | | |
| 90 | 3-[2-(difluoromethoxy)-3,4-difluoro- | A | 558.466 | 559 | 3.45 |
| | phenyl]-N-(2-ethylsulfonyl-4- | | | | |
| | pyridyl)-4,5-dimethyl-5- | | | | |
| | (trifluoromethyl)tetrahydrofuran-2- | | | | |
| | carboxamide | | | | |
| 91 | 3-[2-(difluoromethoxy)-3,4-difluoro- | A | 496.375 | 497.1 | 3.19 |
| | phenyl]-N-[2-(hydroxymethyl)-4- | | | | |
| | pyridyl]-4,5-dimethyl-5- | | | | |
| | (trifluoromethyl)tetrahydrofuran-2- | | | | |
| | carboxamide | | | | |
| 92 | 3-[2-(difluoromethoxy)-3,4-difluoro- | A | 496.375 | 497.6 | 3.91 |
| | phenyl]-N-[2-(hydroxymethyl)-4- | | | | |
| | pyridyl]-4,5-dimethyl-5- | | | | |
| | (trifluoromethyl)tetrahydrofuran-2- | | | | |
| | carboxamide | | | | |
| 93 | 5-[[3-[2-(difluoromethoxy)-3,4- | A | 509.374 | 510.1 | 3.2 |
| | difluoro-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 94 | 5-[[3-[2-(difluoromethoxy)-3,4- | D | 509.374 | 510.1 | 3.2 |
| | difluoro-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 95 | 3-[2-(difluoromethoxy)-3,4-difluoro- | A | 544.44 | 545.1 | 3.31 |
| | phenyl]-4,5-dimethyl-N-(5- | | | | |
| | methylsulfonyl-3-pyridyl)-5- | | | | |
| | (trifluoromethyl)tetrahydrofuran-2- | | | | |
| | carboxamide | | | | |
| 96 | 3-[2-(difluoromethoxy)-3,4-difluoro- | A | 544.44 | 545.1 | 3.3 |
| | phenyl]-4,5-dimethyl-N-(5- | | | | |
| | methylsulfonyl-3-pyridyl)-5- | | | | |
| | (trifluoromethyl)tetrahydrofuran-2- | | | | |
| | carboxamide | | | | |
| 97 | 3-[2-(difluoromethoxy)-4-fluoro-3- | A | 540.476 | 541.1 | 1 |
| | methyl-phenyl]-4,5-dimethyl-N-(2- | | | | |
| | methylsulfonyl-4-pyridyl)-5- | | | | |
| | (trifluoromethyl)tetrahydrofuran-2- | | | | |
| | carboxamide | | | | |
| 98 | 3-[2-(difluoromethoxy)-4-fluoro-3- | A | 492.411 | 493.1 | 3.22 |
| | methyl-phenyl]-4,5-dimethyl-N-(1- | | | | |
| | methyl-2-oxo-4-pyridyl)-5- | | | | |
| | (trifluoromethyl)tetrahydrofuran-2- | | | | |
| | carboxamide | | | | |
| 99 | 3-[2-(difluoromethoxy)-4-fluoro-3- | A | 492.411 | 493.1 | 3.23 |
| | methyl-phenyl]-4,5-dimethyl-N-(1- | | | | |
| | methyl-2-oxo-4-pyridyl)-5- | | | | |
| | (trifluoromethyl)tetrahydrofuran-2- | | | | |
| | carboxamide | | | | |
| 100 | 3-[2-(difluoromethoxy)-4-fluoro-3- | A | 492.411 | 493.1 | 3.22 |
| | methyl-phenyl]-4,5-dimethyl-N-(1- | | | | |
| | methyl-2-oxo-4-pyridyl)-5- | | | | |
| | (trifluoromethyl)tetrahydrofuran-2- | | | | |
| | carboxamide | | | | |
| 101 | 3-(2-ethoxy-3,4-difluoro-phenyl)-4,5- | A | 474.421 | 475.1 | 3.21 |
| | dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 102 | 3-(2-ethoxy-3,4-difluoro-phenyl)-4,5- | A | 474.421 | 475.6 | 3.24 |
| | dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 103 | 3-(2-ethoxy-3,4-difluoro-phenyl)-4,5- | A | 474.421 | 475.6 | 3.23 |
| | dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 104 | (2R,3S,4S,5R)-N-[2-(1,2- | A | 504.447 | 505.4 | 3.13 |
| | dihydroxyethyl)-4-pyridyl]-3-(2- | | | | |
| | ethoxy-3,4-difluoro-phenyl)-4,5- | | | | |
| | dimethyl-5- | | | | |
| | (trifluoromethyl)tetrahydrofuran-2- | | | | |
| | carboxamide | | | | |
| 105 | (2R,3S,4S,5R)-N-[2-(1,2- | A | 504.447 | 505.4 | 3.13 |
| | dihydroxyethyl)-4-pyridyl]-3-(2- | | | | |
| | ethoxy-3,4-difluoro-phenyl)-4,5- | | | | |
| | dimethyl-5- | | | | |
| | (trifluoromethyl)tetrahydrofuran-2- | | | | |
| | carboxamide | | | | |
| 106 | 3-(2-ethoxy-3,4-difluoro-phenyl)-4,5- | A | 522.485 | 523.1 | 3.39 |
| | dimethyl-N-(5-methylsulfonyl-3- | | | | |
| | pyridyl)-5- | | | | |
| | (trifluoromethyl)tetrahydrofuran-2- | | | | |
| | carboxamide | | | | |
| 107 | 3-(2-ethoxy-3,4-difluoro-phenyl)-4,5- | A | 522.485 | 523.1 | 3.39 |
| | dimethyl-N-(5-methylsulfonyl-3- | | | | |
| | pyridyl)-5- | | | | |
| | (trifluoromethyl)tetrahydrofuran-2- | | | | |
| | carboxamide | | | | |
| 108 | 3-(2-ethoxy-3,4-difluoro-phenyl)-4,5- | A | 522.485 | 523.6 | 3.53 |
| | dimethyl-N-(2-methylsulfonyl-4- | | | | |
| | pyridyl)-5- | | | | |
| | (trifluoromethyl)tetrahydrofuran-2- | | | | |
| | carboxamide | | | | |
| 109 | 3-(2-ethoxy-3,4-difluoro-phenyl)-4,5- | A | 521.501 | 522.6 | 3.26 |
| | dimethyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 110 | 3-(2-ethoxy-3,4-difluoro-phenyl)-4,5- | A | 521.501 | 522.6 | 3.26 |
| | dimethyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 111 | 3-(2-ethoxy-3,4-difluoro-phenyl)-4,5- | A | 521.501 | 522.5 | 3.26 |
| | dimethyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 112 | 3-(2-ethoxy-3,4-difluoro-phenyl)-4,5- | A | 521.501 | 522.5 | 3.25 |
| | dimethyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 113 | N-[2-(N,S-dimethylsulfonimidoyl)-4- | A | 535.527 | 536.5 | 3.37 |
| | pyridyl]-3-(2-ethoxy-3,4-difluoro- | | | | |
| | phenyl)-4,5-dimethyl-5- | | | | |
| | (trifluoromethyl)tetrahydrofuran-2- | | | | |
| | carboxamide | | | | |
| 114 | 3-(2-ethoxy-3,4-difluoro-phenyl)-4,5- | A | 536.535 | 536.1 | 3.77 |
| | dimethyl-N-[1-methyl-5- | | | | |
| | (methylsulfonimidoyl)pyridin-1-ium- | | | | |
| | 3-yl]-5- | | | | |
| | (trifluoromethyl)tetrahydrofuran-2- | | | | |
| | carboxamide | | | | |
| 115 | 3-(3,4-difluoro-2-isopropoxy-phenyl)- | A | 536.512 | 537.1 | 3.62 |
| | 4,5-dimethyl-N-(4-methylsulfonyl-2- | | | | |
| | pyridyl)-5- | | | | |
| | (trifluoromethyl)tetrahydrofuran-2- | | | | |
| | carboxamide | | | | |
| 116 | 3-(3,4-difluoro-2-isopropoxy-phenyl)- | A | 536.512 | 537.1 | 3.62 |
| | 4,5-dimethyl-N-(4-methylsulfonyl-2- | | | | |
| | pyridyl)-5- | | | | |
| | (trifluoromethyl)tetrahydrofuran-2- | | | | |
| | carboxamide | | | | |
| 117 | 3-(3,4-difluoro-2-isopropoxy-phenyl)- | A | 535.527 | 536.1 | 3.32 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 118 | 3-(3,4-difluoro-2-isopropoxy-phenyl)- | A | 535.527 | 536.1 | 3.32 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 119 | 3-(3,4-difluoro-2-isopropoxy-phenyl)- | A | 535.527 | 536.1 | 3.32 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 120 | 3-(3,4-difluoro-2-isopropoxy-phenyl)- | A | 535.527 | 536.1 | 3.32 |
| | 4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 121 | 3-(2-ethoxy-4-fluoro-3-methyl- | E | 481.443 | 482.6 | 3.75 |
| | phenyl)-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 122 | 3-(2-ethoxy-4-fluoro-3-methyl- | A | 481.443 | 482.6 | 3.5 |
| | phenyl)-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 123 | 3-(2-ethoxy-4-fluoro-3-methyl- | A | 481.443 | 482.6 | 3.51 |
| | phenyl)-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 124 | 3-(2-ethoxy-4-fluoro-3-methyl- | A | 517.537 | 518.7 | 3.27 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 125 | 3-(2-ethoxy-4-fluoro-3-methyl- | A | 517.537 | 518.6 | 3.3 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 126 | 3-(2-ethoxy-4-fluoro-3-methyl- | A | 517.537 | 518.5 | 3.3 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 127 | 3-(2-ethoxy-4-fluoro-3-methyl- | A | 517.537 | 518.5 | 3.3 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 128 | 3-(2-ethoxy-4-fluoro-3-methyl- | A | 517.537 | 518.6 | 3.26 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 129 | 3-(2-ethoxy-4-fluoro-3-methyl- | A | 517.537 | 518.6 | 3.26 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 130 | N-[2-(N,S-dimethylsulfonimidoyl)-4- | A | 531.563 | 532.2 | 3.38 |
| | pyridyl]-3-(2-ethoxy-4-fluoro-3- | | | | |
| | methyl-phenyl)-4,5-dimethyl-5- | | | | |
| | (trifluoromethyl)tetrahydrofuran-2- | | | | |
| | carboxamide | | | | |
| 131 | N-[2-(N,S-dimethylsulfonimidoyl)-4- | A | 531.563 | 532.2 | 3.39 |
| | pyridyl]-3-(2-ethoxy-4-fluoro-3- | | | | |
| | methyl-phenyl)-4,5-dimethyl-5- | | | | |
| | (trifluoromethyl)tetrahydrofuran-2- | | | | |
| | carboxamide | | | | |
| 132 | 3-(4-fluoro-2-methoxy-3-methyl- | A | 467.417 | 468.6 | 3.35 |
| | phenyl)-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 133 | 3-(4-fluoro-2-methoxy-3-methyl- | A | 467.417 | 468.6 | 3.35 |
| | phenyl)-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 134 | 3-(4-fluoro-2-methoxy-3-methyl- | A | 467.417 | 468.6 | 3.35 |
| | phenyl)-4,5-dimethyl-N- | | | | |
| | (tetrazolo[1,5-a]pyridin-7-yl)-5- | | | | |
| | (trifluoromethyl)tetrahydrofuran-2- | | | | |
| | carboxamide | | | | |
| 135 | N-[2-(1,2-dihydroxyethyl)-4-pyridyl]- | A | 486.457 | 487.6 | 3.04 |
| | 3-(4-fluoro-2-methoxy-3-methyl- | | | | |
| | phenyl)-4,5-dimethyl-5- | | | | |
| | (trifluoromethyl)tetrahydrofuran-2- | | | | |
| | carboxamide | | | | |
| 136 | N-[2-(1,2-dihydroxyethyl)-4-pyridyl]- | A | 486.457 | 487.6 | 3.04 |
| | 3-(4-fluoro-2-methoxy-3-methyl- | | | | |
| | phenyl)-4,5-dimethyl-5- | | | | |
| | (trifluoromethyl)tetrahydrofuran-2- | | | | |
| | carboxamide | | | | |
| 137 | N-[2-(1,2-dihydroxyethyl)-4-pyridyl]- | A | 486.457 | 487.6 | 3.05 |
| | 3-(4-fluoro-2-methoxy-3-methyl- | | | | |
| | phenyl)-4,5-dimethyl-5- | | | | |
| | (trifluoromethyl)tetrahydrofuran-2- | | | | |
| | carboxamide | | | | |
| 138 | N-[2-(1,2-dihydroxyethyl)-1-oxido- | A | 502.456 | 503.7 | 2.89 |
| | pyridin-1-ium-4-yl]-3-(4-fluoro-2- | | | | |
| | methoxy-3-methyl-phenyl)-4,5- | | | | |
| | dimethyl-5- | | | | |
| | (trifluoromethyl)tetrahydrofuran-2- | | | | |
| | carboxamide | | | | |
| 139 | N-[2-(1,2-dihydroxyethyl)-5-fluoro-4- | A | 504.447 | 505.6 | 3.18 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 140 | N-[2-(1,2-dihydroxyethyl)-5-fluoro-4- | A | 504.447 | 505.6 | 3.18 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 141 | N-[2-(1,2-dihydroxyethyl)-5-fluoro-4- | A | 504.447 | 505.6 | 3.18 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 142 | N-[2-(1,2-dihydroxypropyl)-4- | A | 500.483 | 501.5 | 3.15 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 143 | N-[2-(1,2-dihydroxypropyl)-4- | A | 500.483 | 502.4 | 3.15 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 144 | N-[2-(1,2-dihydroxypropyl)-4- | A | 500.483 | 502.7 | 3.16 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 145 | N-[2-(1,2-dihydroxypropyl)-4- | A | 500.483 | 501.6 | 3.15 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 146 | N-[2-(1,2-dihydroxypropyl)-4- | A | 500.483 | 501.5 | 3.16 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 147 | N-[2-(1,2-dihydroxypropyl)-4- | A | 500.483 | 502.6 | 3.16 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 148 | N-[2-(1,2-dihydroxypropyl)-4- | A | 500.483 | 501.6 | 3.15 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 149 | N-[2-(1,2-dihydroxypropyl)-4- | A | 500.483 | 501.6 | 3.15 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 150 | 3-(4-fluoro-2-methoxy-3-methyl- | A | 503.51 | 504.1 | 3.12 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 151 | 3-(4-fluoro-2-methoxy-3-methyl- | A | 503.51 | 504.1 | 3.12 |
| | phenyl)-4,5-dimethyl-N-[2- | | | | |
| | (methylsulfonimidoyl)-4-pyridyl]-5- | | | | |
| | (trifluoromethyl)tetrahydrofuran-2- | | | | |
| | carboxamide | | | | |
| 152 | N-[2-(N,S-dimethylsulfonimidoyl)-4- | A | 517.537 | 518.1 | 3.23 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 153 | N-[2-(N,S-dimethylsulfonimidoyl)-4- | A | 517.537 | 518.1 | 3.23 |
| | pyridyl]-3-(4-fluoro-2-methoxy-3- | | | | |
| | methyl-phenyl)-4,5-dimethyl-5- | | | | |
| | (trifluoromethyl)tetrahydrofuran-2- | | | | |
| | carboxamide | | | | |
| 154 | 3-(3,4-difluorophenyl)-4,5-dimethyl- | A | 478.433 | 479.8 | 3.24 |
| | N-(2-methylsulfonyl-4-pyridyl)-5- | | | | |
| | (trifluoromethyl)tetrahydrofuran-2- | | | | |
| | carboxamide | | | | |
| 155 | 3-(3,4-difluorophenyl)-4,5-dimethyl- | A | 478.433 | 479.1 | 3.23 |
| | N-(2-methylsulfonyl-4-pyridyl)-5- | | | | |
| | (trifluoromethyl)tetrahydrofuran-2- | | | | |
| | carboxamide | | | | |
| 156 | 5-[3-[2-methoxy-3- | A | 505.41 | 506.1 | 3.35 |
| | (trifluoromethyl)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 157 | 5-[[3-[2-methoxy-3- | A | 505.41 | 506.1 | 3.35 |
| | (trifluoromethyl)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 158 | 5-[[3-[4-(difluoromethyl)-3-fluoro-2- | A | 505.41 | 506.3 | 3.26 |
| | methoxy-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 159 | 5-[[3-[4-(difluoromethyl)-3-fluoro-2- | A | 505.41 | 506.3 | 3.26 |
| | methoxy-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 160 | 3-[3-(difluoromethyl)-4-fluoro-2- | A | 502.41 | 503.2 | 3.31 |
| | methoxy-phenyl]-4,5-dimethyl-N- | | | | |
| | (triazolo[1,5-a]pyridin-6-yl)-5- | | | | |
| | (trifluoromethyl)tetrahydrofuran-2- | | | | |
| | carboxamide | | | | |
| 161 | 3-[3-(difluoromethyl)-4-fluoro-2- | A | 502.41 | 503.2 | 3.31 |
| | methoxy-phenyl]-4,5-dimethyl-N- | | | | |
| | (triazolo[1,5-a]pyridin-6-yl)-5- | | | | |
| | (trifluoromethyl)tetrahydrofuran-2- | | | | |
| | carboxamide | | | | |
| 162 | 3-[3-(difluoromethyl)-4-fluoro-2- | C | 574.515 | 575.7 | 3.24 |
| | methoxy-phenyl]-4,5-dimethyl-N-[2- | | | | |
| | (4-methyl-2-oxo-piperazin-1-yl)-4- | | | | |
| | pyridyl]-5- | | | | |
| | (trifluoromethyl)tetrahydrofuran-2- | | | | |
| | carboxamide | | | | |
| 163 | 3-[3-(difluoromethyl)-4-fluoro-2- | A | 574.515 | 575.7 | 3.24 |
| | methoxy-phenyl]-4,5-dimethyl-N-[2- | | | | |
| | (4-methyl-2-oxo-piperazin-1-yl)-4- | | | | |
| | pyridyl]-5- | | | | |
| | (trifluoromethyl)tetrahydrofuran-2- | | | | |
| | carboxamide | | | | |
| 164 | 3-[3-(difluoromethyl)-4-fluoro-2- | A | 512.393 | 513.6 | 3.6 |
| | methoxy-phenyl]-N-[6- | | | | |
| | (difluoromethyl)-3-pyridyl]-4,5- | | | | |
| | dimethyl-5- | | | | |
| | (trifluoromethyl)tetrahydrofuran-2- | | | | |
| | carboxamide | | | | |
| 165 | 3-[3-(difluoromethyl)-4-fluoro-2- | A | 512.393 | 513.6 | 3.6 |
| | methoxy-phenyl]-N-[6- | | | | |
| | (difluoromethyl)-3-pyridyl]-4,5- | | | | |
| | dimethyl-5- | | | | |
| | (trifluoromethyl)tetrahydrofuran-2- | | | | |
| | carboxamide | | | | |
| 166 | 3-[3-(difluoromethyl)-4-fluoro-2- | A | 492.411 | 493.6 | 3.53 |
| | methoxy-phenyl]-N-(6-methoxy-3- | | | | |
| | pyridyl)-4,5-dimethyl-5- | | | | |
| | (trifluoromethyl)tetrahydrofuran-2- | | | | |
| | carboxamide | | | | |
| 167 | 3-[3-(difluoromethyl)-4-fluoro-2- | A | 492.411 | 493.6 | 3.53 |
| | methoxy-phenyl]-N-(6-methoxy-3- | | | | |
| | pyridyl)-4,5-dimethyl-5- | | | | |
| | (trifluoromethyl)tetrahydrofuran-2- | | | | |
| | carboxamide | | | | |
| 168 | 5-[3-[3-(difluoromethyl)-4-fluoro-2- | E [2], A | 505.41 | 506.25 | 3.275 |
| | methoxy-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 169 | 5-[[3-[3-(difluoromethyl)-4-fluoro-2- | E [2], A | 505.41 | 506.25 | 3.275 |
| | methoxy-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 170 | 5-[[3-[3-(difluoromethyl)-4-fluoro-2- | A | 519.437 | 520.4 | 3.3 |
| | methoxy-phenyl]-4,5-dimethyl-5- | | | | |
| | (trifluoromethyl)tetrahydrofuran-2- | | | | |
| | carbonyl]amino]-N-methyl-pyridine- | | | | |
| | 2-carboxamide | | | | |
| 171 | 5-[[3-[3-(difluoromethyl)-4-fluoro-2- | A | 519.437 | 520.4 | 3.31 |
| | methoxy-phenyl]-4,5-dimethyl-5- | | | | |
| | (trifluoromethyl)tetrahydrofuran-2- | | | | |
| | carbonyl]amino]-N-methyl-pyridine- | | | | |
| | 2-carboxamide | | | | |
| 172 | 5-[3-[3-(difluoromethyl)-4-fluoro-2- | A | 506.398 | 507.4 | 3.04 |
| | methoxy-phenyl]-4,5-dimethyl-5- | | | | |
| | (trifluoromethyl)tetrahydrofuran-2- | | | | |
| | carbonyl]amino]pyrimidine-2- | | | | |
| | carboxamide | | | | |
| 173 | 5-[[3-[3-(difluoromethyl)-4-fluoro-2- | A | 506.398 | 507.4 | 3.04 |
| | methoxy-phenyl]-4,5-dimethyl-5- | | | | |
| | (trifluoromethyl)tetrahydrofuran-2- | | | | |
| | carbonyl]amino]pyrimidine-2- | | | | |
| | carboxamide | | | | |
| 174 | 4-[[3-[3-(difluoromethyl)-4-fluoro-2- | A | 506.395 | 507.4 | 2.49 |
| | methoxy-phenyl]-4,5-dimethyl-5- | | | | |
| | (trifluoromethyl)tetrahydrofuran-2- | | | | |
| | carbonyl]amino]pyridine-2-carboxylic | | | | |
| | acid | | | | |
| 175 | 4-[[3-[3-(difluoromethyl)-4-fluoro-2- | A | 506.395 | 507.4 | 2.49 |
| | methoxy-phenyl]-4,5-dimethyl-5- | | | | |
| | (trifluoromethyl)tetrahydrofuran-2- | | | | |
| | carbonyl]amino]pyridine-2-carboxylic | | | | |
| | acid | | | | |
| 176 | 5-((2R,3S,4S,5R)-3-(3- | LCMS | | | |
| | (difluoromethyl)-4-fluoro-2- | | | | |
| | methoxyphenyl)-4,5-dimethyl-5- | | | | |
| | (trifluoromethyl)tetrahydrofuran-2- | | | | |
| | carboxamido)picolinic acid | | | | |
| 177 | 5-((2S,3R,4R,5S)-3-(3- | LCMS | | | |
| | (difluoromethyl)-4-fluoro-2- | | | | |
| | methoxyphenyl)-4,5-dimethyl-5- | | | | |
| | (trifluoromethyl)tetrahydrofuran-2- | | | | |
| | carboxamido)picolinic acid | | | | |
| 178 | 5-[[3-[4-fluoro-2-methoxy-3- | A | 499.455 | 500.6 | 3.12 |
| | (methoxymethyl)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 179 | 5-[[3-[4-fluoro-2-methoxy-3- | A | 499.455 | 500.6 | 3.12 |
| | (methoxymethyl)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 180 | 3-(3,4-difluoro-2-methoxy-phenyl)- | A | 511.463 | 512.5 | 3.4 |
| | 4,5-dimethyl-N-(3-methyl-1- | | | | |
| | methylsulfonyl-pyrazol-4-yl)-5- | | | | |
| | (trifluoromethyl)tetrahydrofuran-2- | | | | |
| | carboxamide | | | | |
| 181 | (2R,3S,4S,5R)-3-(3,4-difluoro-2- | A | 529.5 | 530.8 | 3.29 |
| | methoxy-phenyl)-4,5-dimethyl-N-[2- | | | | |
| | (morpholinomethyl)-4-pyridyl]-5- | | | | |
| | (trifluoromethyl)tetrahydrofuran-2- | | | | |
| | carboxamide | | | | |
| 182 | (2R,3S,4S,5R)-3-(3,4-difluoro-2- | A | 549.481 | 550.8 | 3.63 |
| | methoxy-phenyl)-N-[2-[(3,3- | | | | |
| | difluoropyrrolidin-1-yl)methyl]-4- | | | | |
| | pyridyl]-4,5-dimethyl-5- | | | | |
| | (trifluoromethyl)tetrahydrofuran-2- | | | | |
| | carboxamide | | | | |
| 183 | (2R,3S,4S,5R)-3-(3,4-difluoro-2- | E | 487.463 | 488.6 | 2.8 |
| | methoxy-phenyl)-N-[2- | | | | |
| | [(dimethylamino)methyl]-4-pyridyl]- | | | | |
| | 4,5-dimethyl-5- | | | | |
| | (trifluoromethyl)tetrahydrofuran-2- | | | | |
| | carboxamide | | | | |
| 184 | (2R,3S,4S,5R)-3-(3,4-difluoro-2- | E | 513.5 | 514.7 | 2.86 |
| | methoxy-phenyl)-4,5-dimethyl-N-[2- | | | | |
| | (pyrrolidin-1-ylmethyl)-4-pyridyl]-5- | | | | |
| | (trifluoromethyl)tetrahydrofuran-2- | | | | |
| | carboxamide | | | | |
| 185 | (2R,3S,4S,5R)-3-(3,4-difluoro-2- | A | 483.431 | 484.2 | 3.21 |
| | methoxy-phenyl)-4,5-dimethyl-N-(3- | | | | |
| | methylbenzimidazol-5-yl)-5- | | | | |
| | (trifluoromethyl)tetrahydrofuran-2- | | | | |
| | carboxamide | | | | |
| 186 | (2R,3S,4S,5R)-3-(3,4-difluoro-2- | A | 515.473 | 515.9 | 3.49 |
| | methoxy-phenyl)-4,5-dimethyl-N-(6- | | | | |
| | morpholino-3-pyridyl)-5- | | | | |
| | (trifluoromethyl)tetrahydrofuran-2- | | | | |
| | carboxamide | | | | |
| 187 | (2R,3S,4S,5R)-N-(5-cyano-2-pyridyl)- | A | 455.378 | 456.2 | 3.6 |
| | 3-(3,4-difluoro-2-methoxy-phenyl)- | | | | |
| | 4,5-dimethyl-5- | | | | |
| | (trifluoromethyl)tetrahydrofuran-2- | | | | |
| | carboxamide | | | | |
| 188 | (2R,3S,4S,5R)-3-(3,4-difluoro-2- | A | 509.447 | 510 | 3.24 |
| | methoxy-phenyl)-4,5-dimethyl-N-(2- | | | | |
| | sulfamoyl-4-pyridyl)-5- | | | | |
| | (trifluoromethyl)tetrahydrofuran-2- | | | | |
| | carboxamide | | | | |
| 189 | (2R,3S,4S,5R)-3-(3,4-difluoro-2- | A | 496.375 | 497 | 3.82 |
| | methoxy-phenyl)-N-[2- | | | | |
| | (difluoromethoxy)-4-pyridyl]-4,5- | | | | |
| | dimethyl-5- | | | | |
| | (trifluoromethyl)tetrahydrofuran-2- | | | | |
| | carboxamide | | | | |
| 190 | (2R,3S,4S,5R)-3-(3,4-difluoro-2- | A | 446.368 | 447 | 2.98 |
| | methoxy-phenyl)-4,5-dimethyl-N-(6- | | | | |
| | oxo-1H-pyridin-3-yl)-5- | | | | |
| | (trifluoromethyl)tetrahydrofuran-2- | | | | |
| | carboxamide | | | | |
| 191 | (2R,3S,4S,5R)-N-(2-bromo-5-methyl- | H | 523.291 | 523.09 | 1.16 |
| | 4-pyridyl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 192 | (2R,3S,4S,5R)-N-(6-cyano-3-pyridyl)- | A | 455.378 | 456 | 3.59 |
| | 3-(3,4-difluoro-2-methoxy-phenyl)- | | | | |
| | 4,5-dimethyl-5- | | | | |
| | (trifluoromethyl)tetrahydrofuran-2- | | | | |
| | carboxamide | | | | |
| 193 | (2R,3S,4S,5R)-3-(3,4-difluoro-2- | A | 496.43 | 497 | 3.29 |
| | methoxy-phenyl)-N-[6-(1H-imidazol- | | | | |
| | 2-yl)-3-pyridyl]-4,5-dimethyl-5- | | | | |
| | (trifluoromethyl)tetrahydrofuran-2- | | | | |
| | carboxamide | | | | |
| 194 | (2R,3S,4S,5R)-3-(3,4-difluoro-2- | A | 536.512 | 537.08 | 3.23 |
| | methoxy-phenyl)-4,5-dimethyl-N-[6- | | | | |
| | (2-methylsulfonylethyl)-3-pyridyl]-5- | | | | |
| | (trifluoromethyl)tetrahydrofuran-2- | | | | |
| | carboxamide | | | | |
| 195 | (2R,3S,4S,5R)-3-(3,4-difluoro-2- | A | 530.484 | 532 | 3.69 |
| | methoxy-phenyl)-4,5-dimethyl-N-(2- | | | | |
| | tetrahydropyran-4-yloxy-4-pyridyl)-5- | | | | |
| | (trifluoromethyl)tetrahydrofuran-2- | | | | |
| | carboxamide | | | | |
| 196 | (2R,3S,4S,5R)-3-(3,4-difluoro-2- | A | 420.331 | 422 | 3.24 |
| | methoxy-phenyl)-4,5-dimethyl-N- | | | | |
| | oxazol-2-yl-5- | | | | |
| | (trifluoromethyl)tetrahydrofuran-2- | | | | |
| | carboxamide | | | | |
| 197 | (2R,3S,4S,5R)-3-(3,4-difluoro-2- | A | 543.483 | 544 | 3.39 |
| | methoxy-phenyl)-4,5-dimethyl-N-[2- | | | | |
| | (1-methyl-2-oxo-pyrrolidin-3-yl)oxy- | | | | |
| | 4-pyridyl]-5- | | | | |
| | (trifluoromethyl)tetrahydrofuran-2- | | | | |
| | carboxamide | | | | |
| 198 | (2R,3S,4S,5R)-3-(3,4-difluoro-2- | A | 543.483 | 544 | 3.39 |
| | methoxy-phenyl)-4,5-dimethyl-N-[2- | | | | |
| | (1-methyl-2-oxo-pyrrolidin-3-yl)oxy- | | | | |
| | 4-pyridyl]-5- | | | | |
| | (trifluoromethyl)tetrahydrofuran-2- | | | | |
| | carboxamide | | | | |
| 199 | 3-(3,4-difluoro-2-methoxy-phenyl)-N- | A | 508.411 | 509.6 | 3.14 |
| | [2-(1,2-dihydroxyethyl)-5-fluoro-4- | | | | |
| | pyridyl]-4,5-dimethyl-5- | | | | |
| | (trifluoromethyl)tetrahydrofuran-2- | | | | |
| | carboxamide | | | | |
| 200 | 3-(3,4-difluoro-2-methoxy-phenyl)-N- | A | 508.411 | 509.6 | 3.14 |
| | [2-(1,2-dihydroxyethyl)-5-fluoro-4- | | | | |
| | pyridyl]-4,5-dimethyl-5- | | | | |
| | (trifluoromethyl)tetrahydrofuran-2- | | | | |
| | carboxamide | | | | |
| 201 | (2R,3S,4S,5R)-3-(3,4-difluoro-2- | A [3], A | 507.451 | 508.433 | 2.97 |
| | methoxy-phenyl)-N-[1-[(2R)-2,3- | [2], A | | | |
| | dihydroxypropyl]-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 202 | (2R,3S,4S,5R)-3-(3,4-difluoro-2- | A | 504.447 | | 3.13 |
| | methoxy-phenyl)-N-[2-(1,2- | | | | |
| | dihydroxy-1-methyl-ethyl)-4-pyridyl]- | | | | |
| | 4,5-dimethyl-5- | | | | |
| | (trifluoromethyl)tetrahydrofuran-2- | | | | |
| | carboxamide | | | | |
| 203 | 3-(3,4-difluoro-2-methoxy-phenyl)-N- | A | 504.447 | 505.6 | 3.08 |
| | [2-(1,2-dihydroxypropyl)-4-pyridyl]- | | | | |
| | 4,5-dimethyl-5- | | | | |
| | (trifluoromethyl)tetrahydrofuran-2- | | | | |
| | carboxamide | | | | |
| 204 | 3-(3,4-difluoro-2-methoxy-phenyl)-N- | A | 504.447 | 505.6 | 3.08 |
| | [2-(1,2-dihydroxypropyl)-4-pyridyl]- | | | | |
| | 4,5-dimethyl-5- | | | | |
| | (trifluoromethyl)tetrahydrofuran-2- | | | | |
| | carboxamide | | | | |
| 205 | 3-(3,4-difluoro-2-methoxy-phenyl)-N- | A | 504.447 | 505.6 | 3.1 |
| | [2-(1,2-dihydroxyethyl)-5-methyl-4- | | | | |
| | pyridyl]-4,5-dimethyl-5- | | | | |
| | (trifluoromethyl)tetrahydrofuran-2- | | | | |
| | carboxamide | | | | |
| 206 | 3-(3,4-difluoro-2-methoxy-phenyl)-N- | A | 491.408 | 492.6 | 2.92 |
| | [6-(1,2-dihydroxyethyl)pyridazin-4- | | | | |
| | yl]-4,5-dimethyl-5- | | | | |
| | (trifluoromethyl)tetrahydrofuran-2- | | | | |
| | carboxamide | | | | |
| 207 | 3-(3,4-difluoro-2-methoxy-phenyl)-N- | A | 491.408 | 492.6 | 2.92 |
| | [6-(1,2-dihydroxyethyl)pyridazin-4- | | | | |
| | yl]-4,5-dimethyl-5- | | | | |
| | (trifluoromethyl)tetrahydrofuran-2- | | | | |
| | carboxamide | | | | |
| 208 | 3-(3,4-difluoro-2-methoxy-phenyl)-N- | A | 504.447 | 505.6 | 3.09 |
| | [2-(1,2-dihydroxypropyl)-4-pyridyl]- | | | | |
| | 4,5-dimethyl-5- | | | | |
| | (trifluoromethyl)tetrahydrofuran-2- | | | | |
| | carboxamide | | | | |
| 209 | 3-(3,4-difluoro-2-methoxy-phenyl)-N- | A | 504.447 | 506.6 | 3.09 |
| | [2-(1,2-dihydroxypropyl)-4-pyridyl]- | | | | |
| | 4,5-dimethyl-5- | | | | |
| | (trifluoromethyl)tetrahydrofuran-2- | | | | |
| | carboxamide | | | | |
| 210 | 3-(3,4-difluoro-2-methoxy-phenyl)-N- | A | 506.42 | 507.6 | 2.85 |
| | [2-(1,2-dihydroxyethyl)-1-oxido- | | | | |
| | pyridin-1-ium-4-yl]-4,5-dimethyl-5- | | | | |
| | (trifluoromethyl)tetrahydrofuran-2- | | | | |
| | carboxamide | | | | |
| 211 | (2R,3S,4S,5R)-3-(3,4-difluoro-2- | A | 511.463 | 512 | 3.26 |
| | methoxy-phenyl)-4,5-dimethyl-N-(2- | | | | |
| | methyl-5-methylsulfonyl-pyrazol-3- | | | | |
| | yl)-5- | | | | |
| | (trifluoromethyl)tetrahydrofuran-2- | | | | |
| | carboxamide | | | | |
| 212 | (2R,3S,4S,5R)-N-(3H-benzimidazol- | A | 469.405 | 470 | 3.15 |
| | 5-yl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 213 | (2R,3S,4S,5R)-N-[6-(3-aminooxetan- | A | 501.446 | 502.6 | 3.13 |
| | 3-yl)-3-pyridyl]-3-(3,4-difluoro-2- | | | | |
| | methoxy-phenyl)-4,5-dimethyl-5- | | | | |
| | (trifluoromethyl)tetrahydrofuran-2- | | | | |
| | carboxamide | | | | |
| 214 | (2R,3S,4S,5R)-3-(3,4-difluoro-2- | A | 529.5 | 530.22 | 3.23 |
| | methoxy-phenyl)-4,5-dimethyl-N-[2- | | | | |
| | [[(3S)-morpholin-3-yl]methyl]-4- | | | | |
| | pyridyl]-5- | | | | |
| | (trifluoromethyl)tetrahydrofuran-2- | | | | |
| | carboxamide | | | | |
| 215 | (2R,3S,4S,5R)-3-(3,4-difluoro-2- | A | 527.527 | 528.27 | 3.73 |
| | methoxy-phenyl)-4,5-dimethyl-N-[6- | | | | |
| | (4-piperidylmethyl)-3-pyridyl]-5- | | | | |
| | (trifluoromethyl)tetrahydrofuran-2- | | | | |
| | carboxamide | | | | |
| 216 | (2R,3S,4S,5R)-3-(3,4-difluoro-2- | A | 529.5 | 530.2 | 3.23 |
| | methoxy-phenyl)-4,5-dimethyl-N-[2- | | | | |
| | [[(3R)-morpholin-3-yl]methyl]-4- | | | | |
| | pyridyl]-5- | | | | |
| | (trifluoromethyl)tetrahydrofuran-2- | | | | |
| | carboxamide | | | | |
| 217 | (2R,3S,4S,5R)-N-[6-(1-amino-2- | A | 504.45 | 505.6 | 3.25 |
| | methoxy-ethyl)pyrimidin-4-yl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 218 | (2R,3S,4S,5R)-N-[6-(1-amino-2- | A | 504.45 | 505.6 | 3.25 |
| | methoxy-ethyl)pyrimidin-4-yl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 219 | (2R,3S,4S,5R)-3-(3,4-difluoro-2- | A | 489.436 | 490.2 | 3.26 |
| | methoxy-phenyl)-N-[6-(1-hydroxy-1- | | | | |
| | methyl-ethyl)pyridazin-4-yl]-4,5- | | | | |
| | dimethyl-5- | | | | |
| | (trifluoromethyl)tetrahydrofuran-2- | | | | |
| | carboxamide | | | | |
| 220 | (2R,3S,4S,5R)-3-(3,4-difluoro-2- | A | 516.458 | 517.7 | 3.23 |
| | methoxy-phenyl)-N-[2-(3- | | | | |
| | hydroxytetrahydrofuran-3-yl)-4- | | | | |
| | pyridyl]-4,5-dimethyl-5- | | | | |
| | (trifluoromethyl)tetrahydrofuran-2- | | | | |
| | carboxamide | | | | |
| 221 | (2R,3S,4S,5R)-3-(3,4-difluoro-2- | A | 516.458 | 517.7 | 3.23 |
| | methoxy-phenyl)-N-[2-(3- | | | | |
| | hydroxytetrahydrofuran-3-yl)-4- | | | | |
| | pyridyl]-4,5-dimethyl-5- | | | | |
| | (trifluoromethyl)tetrahydrofuran-2- | | | | |
| | carboxamide | | | | |
| 222 | (2R,3S,4S,5R)-3-(3,4-difluoro-2- | A [2], A | 504.447 | 505.35 | 3.205 |
| | methoxy-phenyl)-N-[2-(1-hydroxy-2- | | | | |
| | methoxy-ethyl)-4-pyridyl]-4,5- | | | | |
| | dimethyl-5- | | | | |
| | (trifluoromethyl)tetrahydrofuran-2- | | | | |
| | carboxamide | | | | |
| 223 | (2R,3S,4S,5R)-3-(3,4-difluoro-2- | A [2], A | 504.447 | 505.35 | 3.205 |
| | methoxy-phenyl)-N-[2-(1-hydroxy-2- | | | | |
| | methoxy-ethyl)-4-pyridyl]-4,5- | | | | |
| | dimethyl-5- | | | | |
| | (trifluoromethyl)tetrahydrofuran-2- | | | | |
| | carboxamide | | | | |
| 224 | (2R,3S,4S,5R)-3-(3,4-difluoro-2- | A | 506.438 | 507.5 | 3.43 |
| | methoxy-phenyl)-N-[6-(2-fluoro-1- | | | | |
| | hydroxy-1-methyl-ethyl)-3-pyridyl]- | | | | |
| | 4,5-dimethyl-5- | | | | |
| | (trifluoromethyl)tetrahydrofuran-2- | | | | |
| | carboxamide | | | | |
| 225 | (2R,3S,4S,5R)-3-(3,4-difluoro-2- | A | 506.438 | 507.4 | 3.43 |
| | methoxy-phenyl)-N-[6-(2-fluoro-1- | | | | |
| | hydroxy-1-methyl-ethyl)-3-pyridyl]- | | | | |
| | 4,5-dimethyl-5- | | | | |
| | (trifluoromethyl)tetrahydrofuran-2- | | | | |
| | carboxamide | | | | |
| 226 | (2R,3S,4S,5R)-3-(3,4-difluoro-2- | A | 506.438 | 507.3 | 3.51 |
| | methoxy-phenyl)-N-[2-(2-fluoro-1- | | | | |
| | hydroxy-1-methyl-ethyl)-4-pyridyl]- | | | | |
| | 4,5-dimethyl-5- | | | | |
| | (trifluoromethyl)tetrahydrofuran-2- | | | | |
| | carboxamide | | | | |
| 227 | (2R,3S,4S,5R)-3-(3,4-difluoro-2- | A | 506.438 | 507.3 | 3.51 |
| | methoxy-phenyl)-N-[2-(2-fluoro-1- | | | | |
| | hydroxy-1-methyl-ethyl)-4-pyridyl]- | | | | |
| | 4,5-dimethyl-5- | | | | |
| | (trifluoromethyl)tetrahydrofuran-2- | | | | |
| | carboxamide | | | | |
| 228 | (2R,3S,4S,5R)-3-(3,4-difluoro-2- | A | 507.426 | 508.2 | 3.48 |
| | methoxy-phenyl)-N-[6-(2-fluoro-1- | | | | |
| | hydroxy-1-methyl-ethyl)pyrimidin-4- | | | | |
| | yl]-4,5-dimethyl-5- | | | | |
| | (trifluoromethyl)tetrahydrofuran-2- | | | | |
| | carboxamide | | | | |
| 229 | (2R,3S,4S,5R)-3-(3,4-difluoro-2- | A | 507.426 | 508.3 | 3.47 |
| | methoxy-phenyl)-N-[6-(2-fluoro-1- | | | | |
| | hydroxy-1-methyl-ethyl)pyrimidin-4- | | | | |
| | yl]-4,5-dimethyl-5- | | | | |
| | (trifluoromethyl)tetrahydrofuran-2- | | | | |
| | carboxamide | | | | |
| 230 | (2R,3S,4S,5R)-3-(3,4-difluoro-2- | E | 517.489 | 518.4 | 2.79 |
| | methoxy-phenyl)-N-[2-[1- | | | | |
| | (dimethylamino)-2-hydroxy-ethyl]-4- | | | | |
| | pyridyl]-4,5-dimethyl-5- | | | | |
| | (trifluoromethyl)tetrahydrofuran-2- | | | | |
| | carboxamide | | | | |
| 231 | (2R,3S,4S,5R)-3-(3,4-difluoro-2- | E | 517.489 | 518 | 2.75 |
| | methoxy-phenyl)-N-[2-[1- | | | | |
| | (dimethylamino)-2-hydroxy-ethyl]-4- | | | | |
| | pyridyl]-4,5-dimethyl-5- | | | | |
| | (trifluoromethyl)tetrahydrofuran-2- | | | | |
| | carboxamide | | | | |
| 232 | (2R,3S,4S,5R)-3-(3,4-difluoro-2- | A | 503.462 | 504.7 | 3.08 |
| | methoxy-phenyl)-N-[2-[2-hydroxy-1- | | | | |
| | (methylamino)ethyl]-4-pyridyl]-4,5- | | | | |
| | dimethyl-5- | | | | |
| | (trifluoromethyl)tetrahydrofuran-2- | | | | |
| | carboxamide | | | | |
| 233 | (2R,3S,4S,5R)-3-(3,4-difluoro-2- | A | 503.462 | 504 | 3.09 |
| | methoxy-phenyl)-N-[2-[2-hydroxy-1- | | | | |
| | (methylamino)ethyl]-4-pyridyl]-4,5- | | | | |
| | dimethyl-5- | | | | |
| | (trifluoromethyl)tetrahydrofuran-2- | | | | |
| | carboxamide | | | | |
| 234 | 6-[[(2R,3S,4S,5R)-3-(3,4-difluoro-2- | E | 512.429 | 514 | 2.97 |
| | methoxy-phenyl)-4,5-dimethyl-5- | | | | |
| | (trifluoromethyl)tetrahydrofuran-2- | | | | |
| | carbonyl]amino]imidazo [1,2- | | | | |
| | alpyridine-2-carboxamide | | | | |
| 235 | (2R,3S,4S,5R)-3-(3,4-difluoro-2- | A | 509.447 | 510 | 3.26 |
| | methoxy-phenyl)-4,5-dimethyl-N-(6- | | | | |
| | sulfamoyl-3-pyridyl)-5- | | | | |
| | (trifluoromethyl)tetrahydrofuran-2- | | | | |
| | carboxamide | | | | |
| 236 | (2R,3S,4S,5R)-3-(3,4-difluoro-2- | A | 523.473 | 524 | 3.4 |
| | methoxy-phenyl)-4,5-dimethyl-N-[6- | | | | |
| | (methylsulfamoyl)-3-pyridyl]-5- | | | | |
| | (trifluoromethyl)tetrahydrofuran-2- | | | | |
| | carboxamide | | | | |
| 237 | (2R,3S,4S,5R)-3-(3,4-difluoro-2- | A | 475.409 | 476 | 3.1 |
| | methoxy-phenyl)-N-[2-(2- | | | | |
| | hydroxyethyl)pyrimidin-5-yl]-4,5- | | | | |
| | dimethyl-5- | | | | |
| | (trifluoromethyl)tetrahydrofuran-2- | | | | |
| | carboxamide | | | | |
| 238 | (2R,3S,4S,5R)-3-(3,4-difluoro-2- | A | 533.445 | 534 | 3.31 |
| | methoxy-phenyl)-N-[6-(4- | | | | |
| | hydroxytetrahydrofuran-3- | | | | |
| | yl)oxypyrimidin-4-yl]-4,5-dimethyl-5- | | | | |
| | (trifluoromethyl)tetrahydrofuran-2- | | | | |
| | carboxamide | | | | |
| 239 | (2R,3S,4S,5R)-3-(3,4-difluoro-2- | A | 532.457 | 534 | 3.35 |
| | methoxy-phenyl)-N-[2-(4- | | | | |
| | hydroxytetrahydrofuran-3-yl)oxy-4- | | | | |
| | pyridyl]-4,5-dimethyl-5- | | | | |
| | (trifluoromethyl)tetrahydrofuran-2- | | | | |
| | carboxamide | | | | |
| 240 | (2R,3S,4S,5R)-3-(3,4-difluoro-2- | A | 532.457 | 534 | 3.35 |
| | methoxy-phenyl)-N-[2-(4- | | | | |
| | hydroxytetrahydrofuran-3-yl)oxy-4- | | | | |
| | pyridyl]-4,5-dimethyl-5- | | | | |
| | (trifluoromethyl)tetrahydrofuran-2- | | | | |
| | carboxamide | | | | |
| 241 | (2R,3S,4S,5R)-3-(3,4-difluoro-2- | A | 533.445 | 533 | 3.29 |
| | methoxy-phenyl)-N-[2-(4- | | | | |
| | hydroxytetrahydrofuran-3- | | | | |
| | yl)oxypyrimidin-4-yl]-4,5-dimethyl-5- | | | | |
| | (trifluoromethyl)tetrahydrofuran-2- | | | | |
| | carboxamide | | | | |
| 242 | (2R,3S,4S,5R)-3-(3,4-difluoro-2- | A | 533.445 | 534 | 3.28 |
| | methoxy-phenyl)-N-[2-(4- | | | | |
| | hydroxytetrahydrofuran-3- | | | | |
| | yl)oxypyrimidin-4-yl]-4,5-dimethyl-5- | | | | |
| | (trifluoromethyl)tetrahydrofuran-2- | | | | |
| | carboxamide | | | | |
| 243 | 3-(3,4-difluoro-2-methoxy-phenyl)-N- | A | 474.421 | 475.6 | 3.23 |
| | [2-(hydroxymethyl)-5-methyl-4- | | | | |
| | pyridyl]-4,5-dimethyl-5- | | | | |
| | (trifluoromethyl)tetrahydrofuran-2- | | | | |
| | carboxamide | | | | |
| 244 | (2R,3S,4S,5R)-3-(3,4-difluoro-2- | A | 521.478 | 522.3 | 3.12 |
| | methoxy-phenyl)-N-[1-[(2R)-2- | | | | |
| | hydroxy-3-methoxy-propyl]-3- | | | | |
| | methyl-pyrazol-4-yl]-4,5-dimethyl-5- | | | | |
| | (trifluoromethyl)tetrahydrofuran-2- | | | | |
| | carboxamide | | | | |
| 245 | (2R,3S,4S,5R)-3-(3,4-difluoro-2- | A | 521.478 | 522.3 | 3.12 |
| | methoxy-phenyl)-N-[1-[(2S)-2- | | | | |
| | hydroxy-3-methoxy-propyl]-3- | | | | |
| | methyl-pyrazol-4-yl]-4,5-dimethyl-5- | | | | |
| | (trifluoromethyl)tetrahydrofuran-2- | | | | |
| | carboxamide | | | | |
| 246 | (2R,3S,4S,5R)-3-(3,4-difluoro-2- | A | 461.383 | 461.9 | 3.14 |
| | methoxy-phenyl)-N-[6- | | | | |
| | (hydroxymethyl)pyrimidin-4-yl]-4,5- | | | | |
| | dimethyl-5- | | | | |
| | (trifluoromethyl)tetrahydrofuran-2- | | | | |
| | carboxamide | | | | |
| 247 | (2R,3S,4S,5R)-3-(3,4-difluoro-2- | A | 490.42 | | 3.32 |
| | methoxy-phenyl)-N-[5- | | | | |
| | (hydroxymethyl)-6-methoxy-3- | | | | |
| | pyridyl]-4,5-dimethyl-5- | | | | |
| | (trifluoromethyl)tetrahydrofuran-2- | | | | |
| | carboxamide | | | | |
| 248 | 3-(3,4-difluoro-2-methoxy-phenyl)-N- | A | 504.447 | 505.6 | 3.18 |
| | [2-(2-hydroxy-1-methoxy-ethyl)-4- | | | | |
| | pyridyl]-4,5-dimethyl-5- | | | | |
| | (trifluoromethyl)tetrahydrofuran-2- | | | | |
| | carboxamide | | | | |
| 249 | 3-(3,4-difluoro-2-methoxy-phenyl)-N- | A | 504.447 | 505.6 | 3.17 |
| | [2-(2-hydroxy-1-methoxy-ethyl)-4- | | | | |
| | pyridyl]-4,5-dimethyl-5- | | | | |
| | (trifluoromethyl)tetrahydrofuran-2- | | | | |
| | carboxamide | | | | |
| 250 | (2R,3S,4S,5R)-N-[2- | A | 500.458 | 501.6 | 3.39 |
| | [cyclopropyl(hydroxy)methyl]-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 251 | (2R,3S,4S,5R)-N-[2- | A | 500.458 | 501.6 | 3.39 |
| | [cyclopropyl(hydroxy)methyl]-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 252 | (2R,3S,4S,5R)-3-(3,4-difluoro-2- | A | 492.411 | 493.3 | 3.32 |
| | methoxy-phenyl)-N-[2-(2-fluoro-1- | | | | |
| | hydroxy-ethyl)-4-pyridyl]-4,5- | | | | |
| | dimethyl-5- | | | | |
| | (trifluoromethyl)tetrahydrofuran-2- | | | | |
| | carboxamide | | | | |
| 253 | (2R,3S,4S,5R)-3-(3,4-difluoro-2- | A | 492.411 | 492.1 | 3.31 |
| | methoxy-phenyl)-N-[2-(2-fluoro-1- | | | | |
| | hydroxy-ethyl)-4-pyridyl]-4,5- | | | | |
| | dimethyl-5- | | | | |
| | (trifluoromethyl)tetrahydrofuran-2- | | | | |
| | carboxamide | | | | |
| 254 | (2R,3S,4S,5R)-N-[2-(2,2-difluoro-1- | A | 510.402 | 511.4 | 3.54 |
| | hydroxy-ethyl)-4-pyridyl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 255 | (2R,3S,4S,5R)-N-[2-(2,2-difluoro-1- | A | 510.402 | 511 | 3.4 |
| | hydroxy-ethyl)-4-pyridyl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 256 | (2R,3S,4S,5R)-3-(3,4-difluoro-2- | A | 522.437 | 523 | 3.18 |
| | methoxy-phenyl)-N-[2-(1,3- | | | | |
| | dihydroxypropyl)-5-fluoro-4-pyridyl]- | | | | |
| | 4,5-dimethyl-5- | | | | |
| | (trifluoromethyl)tetrahydrofuran-2- | | | | |
| | carboxamide | | | | |
| 257 | (2R,3S,4S,5R)-3-(3,4-difluoro-2- | A | 522.437 | 523 | 3.18 |
| | methoxy-phenyl)-N-[2-(1,3- | | | | |
| | dihydroxypropyl)-5-fluoro-4-pyridyl]- | | | | |
| | 4,5-dimethyl-5- | | | | |
| | (trifluoromethyl)tetrahydrofuran-2- | | | | |
| | carboxamide | | | | |
| 258 | 6-[[(2R,3S,4S,5R)-3-(3,4-difluoro-2- | A | 526.456 | 527 | 3.23 |
| | methoxy-phenyl)-4,5-dimethyl-5- | | | | |
| | (trifluoromethyl)tetrahydrofuran-2- | | | | |
| | carbonyl]amino]-N-methyl- | | | | |
| | imidazo[1,2-a]pyridine-2- | | | | |
| | carboxamide | | | | |
| 259 | (2R,3S,4S,5R)-3-(3,4-difluoro-2- | A | 582.519 | 583 | 3.25 |
| | methoxy-phenyl)-4,5-dimethyl-N-[2- | | | | |
| | (morpholine-4-carbonyl)imidazo[1,2- | | | | |
| | alpyridin-6-yl]-5- | | | | |
| | (trifluoromethyl)tetrahydrofuran-2- | | | | |
| | carboxamide | | | | |
| 260 | 6-[[(2R,3S,4S,5R)-3-(3,4-difluoro-2- | A | 556.482 | 557 | 3.13 |
| | methoxy-phenyl)-4,5-dimethyl-5- | | | | |
| | (trifluoromethyl)tetrahydrofuran-2- | | | | |
| | carbonyl]amino]-N-(2- | | | | |
| | hydroxyethyl)imidazo[1,2-a]pyridine- | | | | |
| | 2-carboxamide | | | | |
| 261 | 6-[[(2R,3S,4S,5R)-3-(3,4-difluoro-2- | A | 540.482 | 541 | 3.24 |
| | methoxy-phenyl)-4,5-dimethyl-5- | | | | |
| | (trifluoromethyl)tetrahydrofuran-2- | | | | |
| | carbonyl]amino]-N,N-dimethyl- | | | | |
| | imidazo[1,2-a]pyridine-2- | | | | |
| | carboxamide | | | | |
| 262 | (2R,3S,4S,5R)-3-(3,4-difluoro-2- | A | 581.534 | 582 | 3.09 |
| | methoxy-phenyl)-4,5-dimethyl-N-[2- | | | | |
| | (piperazine-1-carbonyl)imidazo[1,2- | | | | |
| | alpyridin-6-yl]-5- | | | | |
| | (trifluoromethyl)tetrahydrofuran-2- | | | | |
| | carboxamide | | | | |
| 263 | (2R,3S,4S,5R)-3-(3,4-difluoro-2- | A | 463.398 | 464.6 | 3.13 |
| | methoxy-phenyl)-N-[5- | | | | |
| | (hydroxymethyl)-1-methyl-pyrazol-3- | | | | |
| | yl]-4,5-dimethyl-5- | | | | |
| | (trifluoromethyl)tetrahydrofuran-2- | | | | |
| | carboxamide | | | | |
| 264 | (2R,3S,4S,5R)-3-(3,4-difluoro-2- | A | 499.43 | | 3.15 |
| | methoxy-phenyl)-N-[2- | | | | |
| | (hydroxymethyl)imidazo[1,2- | | | | |
| | alpyridin-6-yl]-4,5-dimethyl-5- | | | | |
| | (trifluoromethyl)tetrahydrofuran-2- | | | | |
| | carboxamide | | | | |
| 265 | (2R,3S,4S,5R)-3-(3,4-difluoro-2- | A | 517.489 | | 3.34 |
| | methoxy-phenyl)-N-[2-[2- | | | | |
| | hydroxyethyl(methyl)amino]methyl]- | | | | |
| | 4-pyridyl]-4,5-dimethyl-5- | | | | |
| | (trifluoromethyl)tetrahydrofuran-2- | | | | |
| | carboxamide | | | | |
| 266 | (2R,3S,4S,5R)-3-(3,4-difluoro-2- | A | 473.436 | 474.4 | 3.26 |
| | methoxy-phenyl)-4,5-dimethyl-N-[2- | | | | |
| | (1-methylpyrazol-4-yl)cyclopropyl]-5- | | | | |
| | (trifluoromethyl)tetrahydrofuran-2- | | | | |
| | carboxamide | | | | |
| 267 | (2R,3S,4S,5R)-3-(3,4-difluoro-2- | A | 473.436 | 474.3 | 3.26 |
| | methoxy-phenyl)-4,5-dimethyl-N-[2- | | | | |
| | (1-methylpyrazol-4-yl)cyclopropyl]-5- | | | | |
| | (trifluoromethyl)tetrahydrofuran-2- | | | | |
| | carboxamide | | | | |
| 268 | (2R,3S,4S,5R)-3-(3,4-difluoro-2- | A | 473.436 | 474.24 | 3.23 |
| | methoxy-phenyl)-4,5-dimethyl-N-[2- | | | | |
| | (1-methylpyrazol-4-yl)cyclopropyl]-5- | | | | |
| | (trifluoromethyl)tetrahydrofuran-2- | | | | |
| | carboxamide | | | | |
| 269 | (2R,3S,4S,5R)-3-(3,4-difluoro-2- | A | 437.401 | 438.23 | 3.21 |
| | methoxy-phenyl)-4,5-dimethyl-N- | | | | |
| | tetrahydropyran-4-yl-5- | | | | |
| | (trifluoromethyl)tetrahydrofuran-2- | | | | |
| | carboxamide | | | | |
| 270 | (2R,3S,4S,5R)-N- | A | 407.375 | 408.23 | 3.48 |
| | (cyclopropylmethyl)-3-(3,4-difluoro- | | | | |
| | 2-methoxy-phenyl)-4,5-dimethyl-5- | | | | |
| | (trifluoromethyl)tetrahydrofuran-2- | | | | |
| | carboxamide | | | | |
| 271 | (2R,3S,4S,5R)-N-(2-amino-1-methyl- | A | 424.362 | 424.9 | 2.93 |
| | 2-oxo-ethyl)-3-(3,4-difluoro-2- | | | | |
| | methoxy-phenyl)-4,5-dimethyl-5- | | | | |
| | (trifluoromethyl)tetrahydrofuran-2- | | | | |
| | carboxamide | | | | |
| 272 | (2R,3S,4S,5R)-3-(3,4-difluoro-2- | A | 447.399 | 448.18 | 2.95 |
| | methoxy-phenyl)-N-[2-(1H-imidazol- | | | | |
| | 2-yl)ethyl]-4,5-dimethyl-5- | | | | |
| | (trifluoromethyl)tetrahydrofuran-2- | | | | |
| | carboxamide | | | | |
| 273 | (2R,3S,4S,5R)-N-(1-acetyl-4- | A | 478.453 | 479.19 | 3.04 |
| | piperidyl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 274 | (2R,3S,4S,5R)-N-(1-acetyl-3- | A | 478.453 | 479.19 | 3.1 |
| | piperidyl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 275 | (2R,3S,4S,5R)-3-(3,4-difluoro-2- | A | 447.399 | 448.23 | 3.13 |
| | methoxy-phenyl)-4,5-dimethyl-N-[1- | | | | |
| | (1H-pyrazol-5-yl)ethyl]-5- | | | | |
| | (trifluoromethyl)tetrahydrofuran-2- | | | | |
| | carboxamide | | | | |
| 276 | (2R,3S,4S,5R)-3-(3,4-difluoro-2- | A | 465.454 | 466.23 | 3.73 |
| | methoxy-phenyl)-N-[[1- | | | | |
| | (methoxymethyl)cyclobutyl]methyl]- | | | | |
| | 4,5-dimethyl-5- | | | | |
| | (trifluoromethyl)tetrahydrofuran-2- | | | | |
| | carboxamide | | | | |
| 277 | (2R,3S,4S,5R)-N-(cyanomethyl)-3- | A | 392.32 | 392.94 | 3.21 |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 278 | (2R,3S,4S,5R)-3-(3,4-difluoro-2- | A | 422.39 | 423.23 | 3.09 |
| | methoxy-phenyl)-4,5-dimethyl-N-(1- | | | | |
| | methylazetidin-3-yl)-5- | | | | |
| | (trifluoromethyl)tetrahydrofuran-2- | | | | |
| | carboxamide | | | | |
| 279 | (2R,3S,4S,5R)-3-(3,4-difluoro-2- | A | 437.401 | 438.18 | 3.17 |
| | methoxy-phenyl)-4,5-dimethyl-N- | | | | |
| | (tetrahydrofuran-3-ylmethyl)-5- | | | | |
| | (trifluoromethyl)tetrahydrofuran-2- | | | | |
| | carboxamide | | | | |
| 280 | (2R,3S,4S,5R)-N-(3,3- | A | 443.356 | 444.38 | 3.52 |
| | difluorocyclobutyl)-3-(3,4-difluoro-2- | | | | |
| | methoxy-phenyl)-4,5-dimethyl-5- | | | | |
| | (trifluoromethyl)tetrahydrofuran-2- | | | | |
| | carboxamide | | | | |
| 281 | (2R,3S,4S,5R)-3-(3,4-difluoro-2- | A | 450.4 | 451.18 | 2.81 |
| | methoxy-phenyl)-4,5-dimethyl-N-[(5- | | | | |
| | oxopyrrolidin-3-yl)methyl]-5- | | | | |
| | (trifluoromethyl)tetrahydrofuran-2- | | | | |
| | carboxamide | | | | |
| 282 | (2R,3S,4S,5R)-N-(3-amino-2,2- | A | 452.416 | 453.23 | 2.96 |
| | dimethyl-3-oxo-propyl)-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 283 | (2R,3S,4S,5R)-3-(3,4-difluoro-2- | A | 461.426 | 462.23 | 3.12 |
| | methoxy-phenyl)-4,5-dimethyl-N-[2- | | | | |
| | (1-methylpyrazol-4-yl)ethyl]-5- | | | | |
| | (trifluoromethyl)tetrahydrofuran-2- | | | | |
| | carboxamide | | | | |
| 284 | (2R,3S,4S,5R)-3-(3,4-difluoro-2- | A | 465.454 | 465.91 | 3.4 |
| | methoxy-phenyl)-N-[(5,5- | | | | |
| | dimethyltetrahydrofuran-3-yl)methyl]- | | | | |
| | 4,5-dimethyl-5- | | | | |
| | (trifluoromethyl)tetrahydrofuran-2- | | | | |
| | carboxamide | | | | |
| 285 | (2R,3S,4S,5R)-3-(3,4-difluoro-2- | A | 475.452 | 476.19 | 3.13 |
| | methoxy-phenyl)-N-[2-(3,5-dimethyl- | | | | |
| | 1H-pyrazol-4-yl)ethyl]-4,5-dimethyl- | | | | |
| | 5-(trifluoromethyl)tetrahydrofuran-2- | | | | |
| | carboxamide | | | | |
| 286 | 5-[[[(2R,3S,4S,5R)-3-(3,4-difluoro-2- | A | 476.394 | 477.19 | 2.94 |
| | methoxy-phenyl)-4,5-dimethyl-5- | | | | |
| | (trifluoromethyl)tetrahydrofuran-2- | | | | |
| | carbonyl]amino]methyl]furan-3- | | | | |
| | carboxamide | | | | |
| 287 | (2R,3S,4S,5R)-3-(3,4-difluoro-2- | A | 476.437 | 477.14 | 3.38 |
| | methoxy-phenyl)-N-[2-(3,5- | | | | |
| | dimethylisoxazol-4-yl)ethyl]-4,5- | | | | |
| | dimethyl-5- | | | | |
| | (trifluoromethyl)tetrahydrofuran-2- | | | | |
| | carboxamide | | | | |
| 288 | (2R,3S,4S,5R)-3-(3,4-difluoro-2- | A | 477.425 | 478.24 | 3.05 |
| | methoxy-phenyl)-N-[[5- | | | | |
| | (methoxymethyl)-1H-pyrazol-3- | | | | |
| | yl]methyl]-4,5-dimethyl-5- | | | | |
| | (trifluoromethyl)tetrahydrofuran-2- | | | | |
| | carboxamide | | | | |
| 289 | (2R,3S,4S,5R)-3-(3,4-difluoro-2- | A | 477.425 | 478.19 | 3.55 |
| | methoxy-phenyl)-N-[(3-isopropyl- | | | | |
| | 1,2,4-oxadiazol-5-yl)methyl]-4,5- | | | | |
| | dimethyl-5- | | | | |
| | (trifluoromethyl)tetrahydrofuran-2- | | | | |
| | carboxamide | | | | |
| 290 | (2R,3S,4S,5R)-3-(3,4-difluoro-2- | A | 480.426 | 481.24 | 2.95 |
| | methoxy-phenyl)-4,5-dimethyl-N-[(4- | | | | |
| | methyl-5-oxo-morpholin-2- | | | | |
| | yl)methyl]-5- | | | | |
| | (trifluoromethyl)tetrahydrofuran-2- | | | | |
| | carboxamide | | | | |
| 291 | (2R,3S,4S,5R)-N-(1- | A | 483.471 | 484.19 | 3.81 |
| | benzylcyclopropyl)-3-(3,4-difluoro-2- | | | | |
| | methoxy-phenyl)-4,5-dimethyl-5- | | | | |
| | (trifluoromethyl)tetrahydrofuran-2- | | | | |
| | carboxamide | | | | |
| 292 | (2R,3S,4S,5R)-N-[2-(2-cyanoethyl)-5- | A | 486.435 | 487.19 | 3.3 |
| | methyl-pyrazol-3-yl]-3-(3,4-difluoro- | | | | |
| | 2-methoxy-phenyl)-4,5-dimethyl-5- | | | | |
| | (trifluoromethyl)tetrahydrofuran-2- | | | | |
| | carboxamide | | | | |
| 293 | (2R,3S,4S,5R)-3-(3,4-difluoro-2- | A | 493.51 | 494.24 | 3.21 |
| | methoxy-phenyl)-4,5-dimethyl-N-[2- | | | | |
| | (4-methylpiperazin-1-yl)propyl]-5- | | | | |
| | (trifluoromethyl)tetrahydrofuran-2- | | | | |
| | carboxamide | | | | |
| 294 | (2R,3S,4S,5R)-3-(3,4-difluoro-2- | A | 494.454 | 495.14 | 3.29 |
| | methoxy-phenyl)-4,5-dimethyl-N-(6- | | | | |
| | quinolylmethyl)-5- | | | | |
| | (trifluoromethyl)tetrahydrofuran-2- | | | | |
| | carboxamide | | | | |
| 295 | (2R,3S,4S,5R)-3-(3,4-difluoro-2- | A | 458.422 | 459.23 | 3.18 |
| | methoxy-phenyl)-4,5-dimethyl-N-[(4- | | | | |
| | methyl-3-pyridyl)methyl]-5- | | | | |
| | (trifluoromethyl)tetrahydrofuran-2- | | | | |
| | carboxamide | | | | |
| 296 | (2R,3S,4S,5R)-3-(3,4-difluoro-2- | A | 461.426 | 462.23 | 3.18 |
| | methoxy-phenyl)-4,5-dimethyl-N-[1- | | | | |
| | (1-methylpyrazol-4-yl)ethyl]-5- | | | | |
| | (trifluoromethyl)tetrahydrofuran-2- | | | | |
| | carboxamide | | | | |
| 297 | (2R,3S,4S,5R)-N-[2- | A | 464.426 | 465.28 | 3.16 |
| | (cyclopropylamino)-1-methyl-2-oxo- | | | | |
| | ethyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 298 | (2R,3S,4S,5R)-N-(1-acetylpyrrolidin- | A | 464.426 | 465.28 | 2.94 |
| | 3-yl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 299 | (2R,3S,4S,5R)-3-(3,4-difluoro-2- | A | 464.426 | 465.34 | 3.18 |
| | methoxy-phenyl)-4,5-dimethyl-N-(2- | | | | |
| | oxoazepan-3-yl)-5- | | | | |
| | (trifluoromethyl)tetrahydrofuran-2- | | | | |
| | carboxamide | | | | |
| 300 | (2R,3S,4S,5R)-3-(3,4-difluoro-2- | A | 465.454 | 466.39 | 3.35 |
| | methoxy-phenyl)-4,5-dimethyl-N-[(4- | | | | |
| | methyltetrahydropyran-4-yl)methyl]- | | | | |
| | 5-(trifluoromethyl)tetrahydrofuran-2- | | | | |
| | carboxamide | | | | |
| 301 | (2R,3S,4S,5R)-3-(3,4-difluoro-2- | A | 477.425 | 478.24 | 2.89 |
| | methoxy-phenyl)-N-[2-hydroxy-2-(1- | | | | |
| | methylpyrazol-4-yl)ethyl]-4,5- | | | | |
| | dimethyl-5- | | | | |
| | (trifluoromethyl)tetrahydrofuran-2- | | | | |
| | carboxamide | | | | |
| 302 | (2R,3S,4S,5R)-3-(3,4-difluoro-2- | A | 479.441 | 480.34 | 2.79 |
| | methoxy-phenyl)-4,5-dimethyl-N-[2- | | | | |
| | (3-oxopiperazin-1-yl)ethyl]-5- | | | | |
| | (trifluoromethyl)tetrahydrofuran-2- | | | | |
| | carboxamide | | | | |
| 303 | 4-[[(2R,3S,4S,5R)-3-(3,4-difluoro-2- | A | 479.441 | 480.29 | 2.89 |
| | methoxy-phenyl)-4,5-dimethyl-5- | | | | |
| | (trifluoromethyl)tetrahydrofuran-2- | | | | |
| | carbonyl]amino]piperidine-1- | | | | |
| | carboxamide | | | | |
| 304 | (2R,3S,4S,5R)-3-(3,4-difluoro-2- | A | 480.426 | 480.87 | 3.05 |
| | methoxy-phenyl)-4,5-dimethyl-N-(2- | | | | |
| | morpholino-2-oxo-ethyl)-5- | | | | |
| | (trifluoromethyl)tetrahydrofuran-2- | | | | |
| | carboxamide | | | | |
| 305 | (2R,3S,4S,5R)-3-(3,4-difluoro-2- | A | 483.471 | 484.29 | 3.86 |
| | methoxy-phenyl)-4,5-dimethyl-N-[(1- | | | | |
| | phenylcyclopropyl)methyl]-5- | | | | |
| | (trifluoromethyl)tetrahydrofuran-2- | | | | |
| | carboxamide | | | | |
| 306 | (2R,3S,4S,5R)-3-(3,4-difluoro-2- | A | 485.444 | 486.3 | 3.5 |
| | methoxy-phenyl)-4,5-dimethyl-N-(3- | | | | |
| | phenyloxetan-3-yl)-5- | | | | |
| | (trifluoromethyl)tetrahydrofuran-2- | | | | |
| | carboxamide | | | | |
| 307 | (2R,3S,4S,5R)-3-(3,4-difluoro-2- | A | 486.453 | 487.14 | 3.16 |
| | methoxy-phenyl)-4,5-dimethyl-N-(1- | | | | |
| | methylsulfonylazetidin-3-yl)-5- | | | | |
| | (trifluoromethyl)tetrahydrofuran-2- | | | | |
| | carboxamide | | | | |
| 308 | (2R,3S,4S,5R)-3-(3,4-difluoro-2- | A | 489.451 | 490.35 | 3.87 |
| | methoxy-phenyl)-N-[1-(3-fluoro-4- | | | | |
| | methyl-phenyl)ethyl]-4,5-dimethyl-5- | | | | |
| | (trifluoromethyl)tetrahydrofuran-2- | | | | |
| | carboxamide | | | | |
| 309 | (2R,3S,4S,5R)-3-(3,4-difluoro-2- | A | 457.412 | 458.03 | 3.15 |
| | methoxy-phenyl)-N-(1,1-dioxothietan- | | | | |
| | 3-yl)-4,5-dimethyl-5- | | | | |
| | (trifluoromethyl)tetrahydrofuran-2- | | | | |
| | carboxamide | | | | |
| 310 | (2R,3S,4S,5R)-N-[2-(3-aminooxetan- | A | 502.434 | 503.3 | 3.14 |
| | 3-yl)pyrimidin-4-yl]-3-(3,4-difluoro- | | | | |
| | 2-methoxy-phenyl)-4,5-dimethyl-5- | | | | |
| | (trifluoromethyl)tetrahydrofuran-2- | | | | |
| | carboxamide | | | | |
| 311 | (2R,3S,4S,5R)-3-(3,4-difluoro-2- | A | 483.38 | 484.6 | 3.6 |
| | methoxy-phenyl)-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 312 | (2R,3S,4S,5R)-3-(3,4-difluoro-2- | A | 528.515 | 529 | 3.4 |
| | methoxy-phenyl)-4,5-dimethyl-N-[2- | | | | |
| | (4-methylpiperazin-1-yl)-4-pyridyl]-5- | | | | |
| | (trifluoromethyl)tetrahydrofuran-2- | | | | |
| | carboxamide | | | | |
| 313 | (2R,3S,4S,5R)-3-(3,4-difluoro-2- | A | 433.372 | 434 | 3.04 |
| | methoxy-phenyl)-4,5-dimethyl-N-(3- | | | | |
| | methylimidazol-4-yl)-5- | | | | |
| | (trifluoromethyl)tetrahydrofuran-2- | | | | |
| | carboxamide | | | | |
| 314 | (2R,3S,4S,5R)-3-(3,4-difluoro-2- | A | 483.431 | 484 | 3.42 |
| | methoxy-phenyl)-4,5-dimethyl-N-(3- | | | | |
| | methylimidazo[1,5-a]pyridin-6-yl)-5- | | | | |
| | (trifluoromethyl)tetrahydrofuran-2- | | | | |
| | carboxamide | | | | |
| 315 | (2R,3S,4S,5R)-3-(3,4-difluoro-2- | A | 483.431 | 484 | 3.31 |
| | methoxy-phenyl)-4,5-dimethyl-N-(3- | | | | |
| | methylimidazo[1,5-a]pyridin-7-yl)-5- | | | | |
| | (trifluoromethyl)tetrahydrofuran-2- | | | | |
| | carboxamide | | | | |
| 316 | (2R,3S,4S,5R)-3-(3,4-difluoro-2- | A | 510.456 | 512 | 3.34 |
| | methoxy-phenyl)-4,5-dimethyl-N-[2- | | | | |
| | (2-methylimidazol-1-yl)-4-pyridyl]-5- | | | | |
| | (trifluoromethyl)tetrahydrofuran-2- | | | | |
| | carboxamide | | | | |
| 317 | (2R,3S,4S,5R)-3-(3,4-difluoro-2- | A | 473.436 | 474 | 3.19 |
| | methoxy-phenyl)-4,5-dimethyl-N- | | | | |
| | (5,6,7,8-tetrahydroimidazo[1,2- | | | | |
| | alpyridin-3-yl)-5- | | | | |
| | (trifluoromethyl)tetrahydrofuran-2- | | | | |
| | carboxamide | | | | |
| 318 | (2R,3S,4S,5R)-3-(3,4-difluoro-2- | A | 433.372 | 434 | 3.11 |
| | methoxy-phenyl)-4,5-dimethyl-N-(1- | | | | |
| | methylimidazol-4-yl)-5- | | | | |
| | (trifluoromethyl)tetrahydrofuran-2- | | | | |
| | carboxamide | | | | |
| 319 | (2R,3S,4S,5R)-3-(3,4-difluoro-2- | A | 447.399 | 448 | 3.08 |
| | methoxy-phenyl)-N-(3,5- | | | | |
| | dimethylimidazol-4-yl)-4,5-dimethyl- | | | | |
| | 5-(trifluoromethyl)tetrahydrofuran-2- | | | | |
| | carboxamide | | | | |
| 320 | (2R,3S,4S,5R)-3-(3,4-difluoro-2- | A | 473.436 | 474 | 3.54 |
| | methoxy-phenyl)-N-[2- | | | | |
| | (dimethylamino)-4-pyridyl]-4,5- | | | | |
| | dimethyl-5- | | | | |
| | (trifluoromethyl)tetrahydrofuran-2- | | | | |
| | carboxamide | | | | |
| 321 | (2R,3S,4S,5R)-3-(3,4-difluoro-2- | A | 475.409 | 476.3 | 3.27 |
| | methoxy-phenyl)-N-(6,7-dihydro-4H- | | | | |
| | pyrazolo[5,1-c][1,4]oxazin-2-yl)-4,5- | | | | |
| | dimethyl-5- | | | | |
| | (trifluoromethyl)tetrahydrofuran-2- | | | | |
| | carboxamide | | | | |
| 322 | (2R,3S,4S,5R)-3-(3,4-difluoro-2- | A | 447.399 | 448 | 3.04 |
| | methoxy-phenyl)-N-(1,5- | | | | |
| | dimethylimidazol-4-yl)-4,5-dimethyl- | | | | |
| | 5-(trifluoromethyl)tetrahydrofuran-2- | | | | |
| | carboxamide | | | | |
| 323 | [(2R,3S,4S,5R)-3-(3,4-difluoro-2- | A | 456.406 | 457.3 | 3.29 |
| | methoxy-phenyl)-4,5-dimethyl-5- | | | | |
| | (trifluoromethyl)tetrahydrofuran-2- | | | | |
| | yl]-(1,3-dihydropyrrolo[3,4-c]pyridin- | | | | |
| | 2-yl)methanone | | | | |
| 324 | (2R,3S,4S,5R)-3-(3,4-difluoro-2- | A | 476.46 | 477.1 | 3.68 |
| | methoxy-phenyl)-4,5-dimethyl-N-(6- | | | | |
| | methylsulfanyl-3-pyridyl)-5- | | | | |
| | (trifluoromethyl)tetrahydrofuran-2- | | | | |
| | carboxamide | | | | |
| 325 | (2R,3S,4S,5R)-3-(3,4-difluoro-2- | A | 517.489 | 518.3 | 3.31 |
| | methoxy-phenyl)-4,5-dimethyl-N-(3- | | | | |
| | methyl-1-tetrahydropyran-4-yl- | | | | |
| | pyrazol-4-yl)-5- | | | | |
| | (trifluoromethyl)tetrahydrofuran-2- | | | | |
| | carboxamide | | | | |
| 326 | (2R,3S,4S,5R)-3-(3,4-difluoro-2- | A | 517.489 | 518.3 | 3.28 |
| | methoxy-phenyl)-4,5-dimethyl-N-(5- | | | | |
| | methyl-1-tetrahydropyran-4-yl- | | | | |
| | pyrazol-4-yl)-5- | | | | |
| | (trifluoromethyl)tetrahydrofuran-2- | | | | |
| | carboxamide | | | | |
| 327 | (2R,3S,4S,5R)-3-(3,4-difluoro-2- | A | 450.423 | 451.1 | 3.48 |
| | methoxy-phenyl)-4,5-dimethyl-N-(3- | | | | |
| | methylisothiazol-4-yl)-5- | | | | |
| | (trifluoromethyl)tetrahydrofuran-2- | | | | |
| | carboxamide | | | | |
| 328 | (2R,3S,4S,5R)-3-(3,4-difluoro-2- | A | 470.393 | 470.9 | 3.01 |
| | methoxy-phenyl)-4,5-dimethyl-N- | | | | |
| | ([1,2,4]triazolo[4,3-a]pyridin-7-yl)-5- | | | | |
| | (trifluoromethyl)tetrahydrofuran-2- | | | | |
| | carboxamide | | | | |
| 329 | (2R,3S,4S,5R)-3-(3,4-difluoro-2- | A | 470.393 | 471.2 | 3.29 |
| | methoxy-phenyl)-4,5-dimethyl-N- | | | | |
| | (triazolo[1,5-a]pyridin-6-yl)-5- | | | | |
| | (trifluoromethyl)tetrahydrofuran-2- | | | | |
| | carboxamide | | | | |
| 330 | (2R,3S,4S,5R)-3-(3,4-difluoro-2- | A | 489.436 | 490.2 | 3.22 |
| | methoxy-phenyl)-4,5-dimethyl-N-[3- | | | | |
| | methyl-1-(oxetan-3-yl)pyrazol-4-yl]- | | | | |
| | 5-(trifluoromethyl)tetrahydrofuran-2- | | | | |
| | carboxamide | | | | |
| 331 | (2R,3S,4S,5R)-3-(3,4-difluoro-2- | A | 489.436 | 490.2 | 3.18 |
| | methoxy-phenyl)-4,5-dimethyl-N-[5- | | | | |
| | methyl-1-(oxetan-3-yl)pyrazol-4-yl]- | | | | |
| | 5-(trifluoromethyl)tetrahydrofuran-2- | | | | |
| | carboxamide | | | | |
| 332 | (2R,3S,4S,5R)-3-(3,4-difluoro-2- | A | 507.451 | 508.4 | 2.96 |
| | methoxy-phenyl)-N-[1-[(2S)-2,3- | | | | |
| | dihydroxypropyl]-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 333 | (2R,3S,4S,5R)-3-(3,4-difluoro-2- | A | 504.447 | 505.3 | 3.02 |
| | methoxy-phenyl)-N-[2-[(2S)-2,3- | | | | |
| | dihydroxypropyl]-4-pyridyl]-4,5- | | | | |
| | dimethyl-5- | | | | |
| | (trifluoromethyl)tetrahydrofuran-2- | | | | |
| | carboxamide | | | | |
| 334 | (2R,3S,4S,5R)-3-(3,4-difluoro-2- | A | 504.447 | 505 | 2.96 |
| | methoxy-phenyl)-N-[6-[(2R)-2,3- | | | | |
| | dihydroxypropyl]-3-pyridyl]-4,5- | | | | |
| | dimethyl-5- | | | | |
| | (trifluoromethyl)tetrahydrofuran-2- | | | | |
| | carboxamide | | | | |
| 335 | (2R,3S,4S,5R)-3-(3,4-difluoro-2- | A | 507.451 | 508.4 | 2.95 |
| | methoxy-phenyl)-N-[5-[(2R)-2,3- | | | | |
| | dihydroxypropyl]-1-methyl-pyrazol-3- | | | | |
| | yl]-4,5-dimethyl-5- | | | | |
| | (trifluoromethyl)tetrahydrofuran-2- | | | | |
| | carboxamide | | | | |
| 336 | (2R,3S,4S,5R)-3-(3,4-difluoro-2- | A | 507.474 | 508.4 | 3.15 |
| | methoxy-phenyl)-4,5-dimethyl-N-[6- | | | | |
| | (methylsulfonimidoyl)-3-pyridyl]-5- | | | | |
| | (trifluoromethyl)tetrahydrofuran-2- | | | | |
| | carboxamide | | | | |
| 337 | (2R,3S,4S,5R)-3-(3,4-difluoro-2- | A | 507.474 | 508.3 | 3.15 |
| | methoxy-phenyl)-4,5-dimethyl-N-[6- | | | | |
| | (methylsulfonimidoyl)-3-pyridyl]-5- | | | | |
| | (trifluoromethyl)tetrahydrofuran-2- | | | | |
| | carboxamide | | | | |
| 338 | (2R,3S,4S,5R)-3-(3,4-difluoro-2- | A | 521.501 | 523 | 3.26 |
| | methoxy-phenyl)-N-[6-(N,S- | | | | |
| | dimethylsulfonimidoyl)-3-pyridyl]- | | | | |
| | 4,5-dimethyl-5- | | | | |
| | (trifluoromethyl)tetrahydrofuran-2- | | | | |
| | carboxamide | | | | |
| 339 | (2R,3S,4S,5R)-3-(3,4-difluoro-2- | A | 521.501 | 522.9 | 3.26 |
| | methoxy-phenyl)-N-[6-(N,S- | | | | |
| | dimethylsulfonimidoyl)-3-pyridyl]- | | | | |
| | 4,5-dimethyl-5- | | | | |
| | (trifluoromethyl)tetrahydrofuran-2- | | | | |
| | carboxamide | | | | |
| 340 | (2R,3S,4S,5R)-N-[2-(3-aminooxetan- | A | 501.446 | 502 | 3.07 |
| | 3-yl)-4-pyridyl]-3-(3,4-difluoro-2- | | | | |
| | methoxy-phenyl)-4,5-dimethyl-5- | | | | |
| | (trifluoromethyl)tetrahydrofuran-2- | | | | |
| | carboxamide | | | | |
| 341 | (2R,3S,4S,5R)-N-[2-(3-aminooxetan- | A [3], A | 519.437 | 520.4 | 3.28 |
| | 3-yl)-5-fluoro-4-pyridyl]-3-(3,4- | [2], A | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 342 | (2R,3S,4S,5R)-3-(3,4-difluoro-2- | E | 515.473 | 516.4 | 2.83 |
| | methoxy-phenyl)-4,5-dimethyl-N-(2- | | | | |
| | morpholin-3-yl-4-pyridyl)-5- | | | | |
| | (trifluoromethyl)tetrahydrofuran-2- | | | | |
| | carboxamide | | | | |
| 343 | (2R,3S,4S,5R)-3-(3,4-difluoro-2- | E | 515.473 | 516.4 | 2.84 |
| | methoxy-phenyl)-4,5-dimethyl-N-(2- | | | | |
| | morpholin-3-yl-4-pyridyl)-5- | | | | |
| | (trifluoromethyl)tetrahydrofuran-2- | | | | |
| | carboxamide | | | | |
| 344 | (2R,3S,4S,5R)-3-(3,4-difluoro-2- | A | 545.542 | 545.8 | 4.08 |
| | methoxy-phenyl)-N-[2-[2-methoxy-2- | | | | |
| | methyl-1-(methylamino)propyl]-4- | | | | |
| | pyridyl]-4,5-dimethyl-5- | | | | |
| | (trifluoromethyl)tetrahydrofuran-2- | | | | |
| | carboxamide | | | | |
| 345 | (2R,3S,4S,5R)-3-(3,4-difluoro-2- | E | 545.542 | 547 | 4.12 |
| | methoxy-phenyl)-N-[2-[2-methoxy-2- | | | | |
| | methyl-1-(methylamino)propyl]-4- | | | | |
| | pyridyl]-4,5-dimethyl-5- | | | | |
| | (trifluoromethyl)tetrahydrofuran-2- | | | | |
| | carboxamide | | | | |
| 346 | (2R,3S,4S,5R)-N-[2-(3- | A | 515.473 | 516 | 3.12 |
| | aminotetrahydrofuran-3-yl)-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 347 | (2R,3S,4S,5R)-N-[2-(3- | A | 515.473 | 516 | 3.12 |
| | aminotetrahydrofuran-3-yl)-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 348 | (2R,3S,4S,5R)-N-[2-(3- | A | 533.463 | 534.1 | 3.32 |
| | aminotetrahydrofuran-3-yl)-5-fluoro- | | | | |
| | 4-pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 349 | (2R,3S,4S,5R)-N-[2-(3- | A | 533.463 | 534.1 | 3.32 |
| | aminotetrahydrofuran-3-yl)-5-fluoro- | | | | |
| | 4-pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 350 | (2R,3S,4S,5R)-3-(3,4-difluoro-2- | A | 533.463 | 534.1 | 3.35 |
| | methoxy-phenyl)-N-(5-fluoro-2- | | | | |
| | morpholin-2-yl-4-pyridyl)-4,5- | | | | |
| | dimethyl-5- | | | | |
| | (trifluoromethyl)tetrahydrofuran-2- | | | | |
| | carboxamide | | | | |
| 351 | (2R,3S,4S,5R)-3-(3,4-difluoro-2- | A | 533.463 | 534.1 | 3.35 |
| | methoxy-phenyl)-N-(5-fluoro-2- | | | | |
| | morpholin-2-yl-4-pyridyl)-4,5- | | | | |
| | dimethyl-5- | | | | |
| | (trifluoromethyl)tetrahydrofuran-2- | | | | |
| | carboxamide | | | | |
| 352 | (2R,3S,4S,5R)-N-[2-(1-amino-2- | A | 517.489 | 518.4 | 3.36 |
| | methoxy-1-methyl-ethyl)-4-pyridyl]- | | | | |
| | 3-(3,4-difluoro-2-methoxy-phenyl)- | | | | |
| | 4,5-dimethyl-5- | | | | |
| | (trifluoromethyl)tetrahydrofuran-2- | | | | |
| | carboxamide | | | | |
| 353 | (2R,3S,4S,5R)-N-[2-(1-amino-2- | A | 517.489 | 518.4 | 3.36 |
| | methoxy-1-methyl-ethyl)-4-pyridyl]- | | | | |
| | 3-(3,4-difluoro-2-methoxy-phenyl)- | | | | |
| | 4,5-dimethyl-5- | | | | |
| | (trifluoromethyl)tetrahydrofuran-2- | | | | |
| | carboxamide | | | | |
| 354 | (2R,3S,4S,5R)-N-[2-(1-amino-2- | A | 535.479 | 536.5 | 3.52 |
| | methoxy-1-methyl-ethyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 355 | (2R,3S,4S,5R)-N-[2-(1-amino-2- | E | 535.479 | 536.5 | 3.52 |
| | methoxy-1-methyl-ethyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 356 | (2R,3S,4S,5R)-N-[2-(1-amino-2- | A | 549.506 | 550.7 | 3.6 |
| | methoxy-2-methyl-propyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 357 | (2R,3S,4S,5R)-N-[2-(1-amino-2- | A | 549.506 | 550.7 | 3.61 |
| | methoxy-2-methyl-propyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 358 | (2R,3S,4S,5R)-N-[2-(1-amino-2- | A | 503.462 | 504.6 | 3.16 |
| | hydroxy-1-methyl-ethyl)-4-pyridyl]-3- | | | | |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 359 | (2R,3S,4S,5R)-N-[2-(1-amino-2- | A | 503.462 | 504.6 | 3.16 |
| | hydroxy-1-methyl-ethyl)-4-pyridyl]-3- | | | | |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 360 | (2R,3S,4S,5R)-N-[2-(1-amino-2- | A | 521.453 | 522.6 | 3.29 |
| | hydroxy-1-methyl-ethyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 361 | (2R,3S,4S,5R)-3-(3,4-difluoro-2- | A | 515.473 | 516 | 3.15 |
| | methoxy-phenyl)-4,5-dimethyl-N-[2- | | | | |
| | [3-(methylamino)oxetan-3-yl]-4- | | | | |
| | pyridyl]-5- | | | | |
| | (trifluoromethyl)tetrahydrofuran-2- | | | | |
| | carboxamide | | | | |
| 362 | (2R,3S,4S,5R)-3-(3,4-difluoro-2- | A | 529.5 | 530 | 3.24 |
| | methoxy-phenyl)-N-[2-[3- | | | | |
| | (dimethylamino)oxetan-3-yl]-4- | | | | |
| | pyridyl]-4,5-dimethyl-5- | | | | |
| | (trifluoromethyl)tetrahydrofuran-2- | | | | |
| | carboxamide | | | | |
| 363 | (2R,3S,4S,5R)-3-(3,4-difluoro-2- | A | 547.49 | 548 | 3.47 |
| | methoxy-phenyl)-N-[2-[3- | | | | |
| | (dimethylamino)oxetan-3-yl]-5- | | | | |
| | fluoro-4-pyridyl]-4,5-dimethyl-5- | | | | |
| | (trifluoromethyl)tetrahydrofuran-2- | | | | |
| | carboxamide | | | | |
| 364 | (2R,3S,4S,5R)-3-(3,4-difluoro-2- | E | 529.5 | 530.3 | 2.87 |
| | methoxy-phenyl)-4,5-dimethyl-N-[2- | | | | |
| | (4-methylmorpholin-3-yl)-4-pyridyl]- | | | | |
| | 5-(trifluoromethyl)tetrahydrofuran-2- | | | | |
| | carboxamide | | | | |
| 365 | (2R,3S,4S,5R)-3-(3,4-difluoro-2- | A | 529.5 | 530.4 | 3.35 |
| | methoxy-phenyl)-4,5-dimethyl-N-[2- | | | | |
| | (4-methylmorpholin-3-yl)-4-pyridyl]- | | | | |
| | 5-(trifluoromethyl)tetrahydrofuran-2- | | | | |
| | carboxamide | | | | |
| 366 | (2R,3S,4S,5R)-3-(3,4-difluoro-2- | A | 547.49 | 548 | 3.55 |
| | methoxy-phenyl)-N-[5-fluoro-2-(4- | | | | |
| | methylmorpholin-2-yl)-4-pyridyl]-4,5- | | | | |
| | dimethyl-5- | | | | |
| | (trifluoromethyl)tetrahydrofuran-2- | | | | |
| | carboxamide | | | | |
| 367 | (2R,3S,4S,5R)-3-(3,4-difluoro-2- | A | 547.49 | 548.1 | 3.53 |
| | methoxy-phenyl)-N-[5-fluoro-2-(4- | | | | |
| | methylmorpholin-2-yl)-4-pyridyl]-4,5- | | | | |
| | dimethyl-5- | | | | |
| | (trifluoromethyl)tetrahydrofuran-2- | | | | |
| | carboxamide | | | | |
| 368 | (2R,3S,4S,5R)-3-(3,4-difluoro-2- | E | 517.489 | 518.4 | 2.77 |
| | methoxy-phenyl)-N-[2-[1- | | | | |
| | (dimethylamino)-2-hydroxy-ethyl]-4- | | | | |
| | pyridyl]-4,5-dimethyl-5- | | | | |
| | (trifluoromethyl)tetrahydrofuran-2- | | | | |
| | carboxamide | | | | |
| 369 | (2R,3S,4S,5R)-3-(3,4-difluoro-2- | A | 518.474 | 519.1 | 3.22 |
| | methoxy-phenyl)-N-[2-[(2S)-2- | | | | |
| | hydroxy-3-methoxy-propyl]-4- | | | | |
| | pyridyl]-4,5-dimethyl-5- | | | | |
| | (trifluoromethyl)tetrahydrofuran-2- | | | | |
| | carboxamide | | | | |
| 370 | (2R,3S,4S,5R)-3-(3,4-difluoro-2- | A [2], A | 535.479 | 536 | 3.82 |
| | methoxy-phenyl)-N-[2-[2- | | | | |
| | (dimethylamino)-1-hydroxy-ethyl]-5- | | | | |
| | fluoro-4-pyridyl]-4,5-dimethyl-5- | | | | |
| | (trifluoromethyl)tetrahydrofuran-2- | | | | |
| | carboxamide | | | | |
| 371 | (2R,3S,4S,5R)-3-(3,4-difluoro-2- | A | 577.516 | 577.8 | 4.1 |
| | methoxy-phenyl)-N-[5-fluoro-2-(1- | | | | |
| | hydroxy-2-morpholino-ethyl)-4- | | | | |
| | pyridyl]-4,5-dimethyl-5- | | | | |
| | (trifluoromethyl)tetrahydrofuran-2- | | | | |
| | carboxamide | | | | |
| 372 | (2R,3S,4S,5R)-3-(3,4-difluoro-2- | E | 420.334 | 421.1 | 2.97 |
| | methoxy-phenyl)-4,5-dimethyl-N- | | | | |
| | (1H-triazol-4-yl)-5- | | | | |
| | (trifluoromethyl)tetrahydrofuran-2- | | | | |
| | carboxamide | | | | |
| 373 | N-cyano-5-[[(2R,3S,4S,5R)-3-(3,4- | A | 498.403 | 499.4 | 2.63 |
| | difluoro-2-methoxy-phenyl)-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 374 | 5-[[(2R,3S,4S,5R)-3-(3,4-difluoro-2- | E | 503.419 | 504.2 | 3.38 |
| | methoxy-phenyl)-4,5-dimethyl-5- | | | | |
| | (trifluoromethyl)tetrahydrofuran-2- | | | | |
| | carbonyl]amino]-N-hydroxy-N- | | | | |
| | methyl-pyridine-2-carboxamide | | | | |
| 375 | 5-[[(2R,3S,4S,5R)-3-(3,4-difluoro-2- | E | 551.484 | 552.3 | 3.64 |
| | methoxy-phenyl)-4,5-dimethyl-5- | | | | |
| | (trifluoromethyl)tetrahydrofuran-2- | | | | |
| | carbonyl]amino]-N-methylsulfonyl- | | | | |
| | pyridine-2-carboxamide | | | | |
| 376 | 5-[[(2R,3S,4S,5R)-3-(3,4-difluoro-2- | A | 565.51 | | 3.55 |
| | methoxy-phenyl)-4,5-dimethyl-5- | | | | |
| | (trifluoromethyl)tetrahydrofuran-2- | | | | |
| | carbonyl]amino]-N-methyl-N- | | | | |
| | methylsulfonyl-pyridine-2- | | | | |
| | carboxamide | | | | |
| 377 | (2R,3S,4S,5R)-3-(3,4-difluoro-2- | A | 535.479 | 536.7 | 3.54 |
| | methoxy-phenyl)-N-[2-[2- | | | | |
| | (dimethylamino)-1-hydroxy-ethyl]-5- | | | | |
| | fluoro-4-pyridyl]-4,5-dimethyl-5- | | | | |
| | (trifluoromethyl)tetrahydrofuran-2- | | | | |
| | carboxamide | | | | |
| 378 | (2R,3S,4S,5R)-3-(3,4-difluoro-2- | G | 517.489 | 518.3 | 2.62 |
| | methoxy-phenyl)-N-[2-[2- | | | | |
| | (dimethylamino)-1-hydroxy-ethyl]-4- | | | | |
| | pyridyl]-4,5-dimethyl-5- | | | | |
| | (trifluoromethyl)tetrahydrofuran-2- | | | | |
| | carboxamide | | | | |
| 379 | (2R,3S,4S,5R)-3-(3,4-difluoro-2- | A [2], A | 542.498 | 543.1 | 3.17 |
| | methoxy-phenyl)-4,5-dimethyl-N-[2- | | | | |
| | (4-methyl-2-oxo-piperazin-1-yl)-4- | | | | |
| | pyridyl]-5- | | | | |
| | (trifluoromethyl)tetrahydrofuran-2- | | | | |
| | carboxamide | | | | |
| 380 | (2R,3S,4S,5R)-3-(3,4-difluoro-2- | A | 592.579 | 593 | 3.4 |
| | methoxy-phenyl)-4,5-dimethyl-N-[5- | | | | |
| | (4-methylpiperazin-1-yl)sulfonyl-3- | | | | |
| | pyridyl]-5- | | | | |
| | (trifluoromethyl)tetrahydrofuran-2- | | | | |
| | carboxamide | | | | |
| 381 | (2R,3S,4S,5R)-3-(3,4-difluoro-2- | A | 461.383 | 462.3 | 3.18 |
| | methoxy-phenyl)-N-[2- | | | | |
| | (hydroxymethyl)pyrimidin-4-yl]-4,5- | | | | |
| | dimethyl-5- | | | | |
| | (trifluoromethyl)tetrahydrofuran-2- | | | | |
| | carboxamide | | | | |
| 382 | (2R,3S,4S,5R)-3-(3,4-difluoro-2- | A | 461.383 | 462.4 | 3.18 |
| | methoxy-phenyl)-N-[6- | | | | |
| | (hydroxymethyl)pyrazin-2-yl]-4,5- | | | | |
| | dimethyl-5- | | | | |
| | (trifluoromethyl)tetrahydrofuran-2- | | | | |
| | carboxamide | | | | |
| 383 | (2R,3S,4S,5R)-3-(3,4-difluoro-2- | E | 460.394 | 461.3 | 3.13 |
| | methoxy-phenyl)-N-[5- | | | | |
| | (hydroxymethyl)-3-pyridyl]-4,5- | | | | |
| | dimethyl-5- | | | | |
| | (trifluoromethyl)tetrahydrofuran-2- | | | | |
| | carboxamide | | | | |
| 384 | (2R,3S,4S,5R)-3-(3,4-difluoro-2- | I | 557.513 | 558.14 | 2.27 |
| | methoxy-phenyl)-N-[6-[(2R)-2,4- | | | | |
| | dimethyl-6-oxo-piperazin-1- | | | | |
| | yl\|pyridazin-4-yl]-4,5-dimethyl-5- | | | | |
| | (trifluoromethyl)tetrahydrofuran-2- | | | | |
| | carboxamide | | | | |
| 385 | (2R,3S,4S,5R)-3-(3,4-difluoro-2- | A | 474.421 | 475.6 | 3.15 |
| | methoxy-phenyl)-N-[2-(2- | | | | |
| | hydroxyethyl)-4-pyridyl]-4,5- | | | | |
| | dimethyl-5- | | | | |
| | (trifluoromethyl)tetrahydrofuran-2- | | | | |
| | carboxamide | | | | |
| 386 | (2R,3S,4S,5R)-3-(3,4-difluoro-2- | A | 543.526 | | 3.33 |
| | methoxy-phenyl)-N-[2-[[(2S)-2- | | | | |
| | (hydroxymethyl)pyrrolidin-1- | | | | |
| | yl]methyl]-4-pyridyl]-4,5-dimethyl-5- | | | | |
| | (trifluoromethyl)tetrahydrofuran-2- | | | | |
| | carboxamide | | | | |
| 387 | (2R,3S,4S,5R)-3-(3,4-difluoro-2- | A | 459.41 | 460 | 3.35 |
| | methoxy-phenyl)-4,5-dimethyl-N-[2- | | | | |
| | (methylamino)-4-pyridyl]-5- | | | | |
| | (trifluoromethyl)tetrahydrofuran-2- | | | | |
| | carboxamide | | | | |
| 388 | (2R,3S,4S,5R)-3-(3,4-difluoro-2- | E | 471.381 | 472.3 | 3.4 |
| | methoxy-phenyl)-4,5-dimethyl-N- | | | | |
| | ([1,2,4]triazolo[4,3-a]pyrimidin-6-yl)- | | | | |
| | 5-(trifluoromethyl)tetrahydrofuran-2- | | | | |
| | carboxamide | | | | |
| 389 | (2R,3S,4S,5R)-3-(3,4-difluoro-2- | F [2], F | 489.436 | 490.235 | 2.67 |
| | methoxy-phenyl)-N-[6-(1-hydroxy-1- | | | | |
| | methyl-ethyl)pyrimidin-4-yl]-4,5- | | | | |
| | dimethyl-5- | | | | |
| | (trifluoromethyl)tetrahydrofuran-2- | | | | |
| | carboxamide | | | | |
| 390 | (2R,3S,4S,5R)-3-(3,4-difluoro-2- | F | 489.436 | 490.22 | 2.59 |
| | methoxy-phenyl)-N-[2-(1-hydroxy-1- | | | | |
| | methyl-ethyl)pyrimidin-5-yl]-4,5- | | | | |
| | dimethyl-5- | | | | |
| | (trifluoromethyl)tetrahydrofuran-2- | | | | |
| | carboxamide | | | | |
| 391 | (2R,3S,4S,5R)-3-(3,4-difluoro-2- | A | 542.498 | 543 | 3.19 |
| | methoxy-phenyl)-4,5-dimethyl-N-[6- | | | | |
| | (4-methyl-2-oxo-piperazin-1-yl)-3- | | | | |
| | pyridyl]-5- | | | | |
| | (trifluoromethyl)tetrahydrofuran-2- | | | | |
| | carboxamide | | | | |
| 392 | (2R,3S,4S,5R)-3-(3,4-difluoro-2- | C | 514.485 | 515.3 | 0.98 |
| | methoxy-phenyl)-4,5-dimethyl-N-[6- | | | | |
| | (tetrahydrofuran-3-ylmethyl)-3- | | | | |
| | pyridyl]-5- | | | | |
| | (trifluoromethyl)tetrahydrofuran-2- | | | | |
| | carboxamide | | | | |
| 393 | (2R,3S,4S,5R)-3-(3,4-difluoro-2- | A | 512.429 | 513.22 | 3.5 |
| | methoxy-phenyl)-4,5-dimethyl-N-[6- | | | | |
| | (3-methyl-1,2,4-oxadiazol-5-yl)-3- | | | | |
| | pyridyl]-5- | | | | |
| | (trifluoromethyl)tetrahydrofuran-2- | | | | |
| | carboxamide | | | | |
| 394 | (2R,3S,4S,5R)-3-(3,4-difluoro-2- | A | 497.415 | 498.12 | 3.37 |
| | methoxy-phenyl)-4,5-dimethyl-N-(6- | | | | |
| | oxazol-5-yl-3-pyridyl)-5- | | | | |
| | (trifluoromethyl)tetrahydrofuran-2- | | | | |
| | carboxamide | | | | |
| 395 | (2R,3S,4S,5R)-3-(3,4-difluoro-2- | B | 510.456 | 511.2 | 3.74 |
| | methoxy-phenyl)-4,5-dimethyl-N-[6- | | | | |
| | (3-methylpyrazol-1-yl)-3-pyridyl]-5- | | | | |
| | (trifluoromethyl)tetrahydrofuran-2- | | | | |
| | carboxamide | | | | |
| 396 | (2R,3S,4S,5R)-3-(3,4-difluoro-2- | A | 507.452 | 508.17 | 3.59 |
| | methoxy-phenyl)-4,5-dimethyl-N-[6- | | | | |
| | (2-pyridyl)-3-pyridyl]-5- | | | | |
| | (trifluoromethyl)tetrahydrofuran-2- | | | | |
| | carboxamide | | | | |
| 397 | (2R,3S,4S,5R)-3-(3,4-difluoro-2- | A | 511.444 | 510.12 | 2.48 |
| | methoxy-phenyl)-4,5-dimethyl-N-[6- | | | | |
| | (4-methyl-1,2,4-triazol-3-yl)-3- | | | | |
| | pyridyl]-5- | | | | |
| | (trifluoromethyl)tetrahydrofuran-2- | | | | |
| | carboxamide | | | | |
| 398 | (2R,3S,4S,5R)-3-(3,4-difluoro-2- | A | 512.429 | 513.12 | 3.31 |
| | methoxy-phenyl)-4,5-dimethyl-N-[6- | | | | |
| | (5-methyl-1,3,4-oxadiazol-2-yl)-3- | | | | |
| | pyridyl]-5- | | | | |
| | (trifluoromethyl)tetrahydrofuran-2- | | | | |
| | carboxamide | | | | |
| 399 | (2R,3S,4S,5R)-3-(3,4-difluoro-2- | A | 497.415 | 498.12 | 3.36 |
| | methoxy-phenyl)-4,5-dimethyl-N-(6- | | | | |
| | oxazol-2-yl-3-pyridyl)-5- | | | | |
| | (trifluoromethyl)tetrahydrofuran-2- | | | | |
| | carboxamide | | | | |
| 400 | (2R,3S,4S,5R)-3-(3,4-difluoro-2- | A | 510.456 | 511.17 | 3.36 |
| | methoxy-phenyl)-4,5-dimethyl-N-[6- | | | | |
| | (1-methylpyrazol-3-yl)-3-pyridyl]-5- | | | | |
| | (trifluoromethyl)tetrahydrofuran-2- | | | | |
| | carboxamide | | | | |
| 401 | (2R,3S,4S,5R)-3-(3,4-difluoro-2- | A | 496.43 | 497.12 | 3.34 |
| | methoxy-phenyl)-N-(6-imidazol-1-yl- | | | | |
| | 3-pyridyl)-4,5-dimethyl-5- | | | | |
| | (trifluoromethyl)tetrahydrofuran-2- | | | | |
| | carboxamide | | | | |
| 402 | (2R,3S,4S,5R)-3-(3,4-difluoro-2- | B | 497.418 | 498.1 | 3.4 |
| | methoxy-phenyl)-4,5-dimethyl-N-[6- | | | | |
| | (1,2,4-triazol-1-yl)-3-pyridyl]-5- | | | | |
| | (trifluoromethyl)tetrahydrofuran-2- | | | | |
| | carboxamide | | | | |
| 403 | (2R,3S,4S,5R)-3-(3,4-difluoro-2- | A | 484.416 | 485.17 | 3.27 |
| | methoxy-phenyl)-N-[6-(3- | | | | |
| | hydroxyprop-1-ynyl)-3-pyridyl]-4,5- | | | | |
| | dimethyl-5- | | | | |
| | (trifluoromethyl)tetrahydrofuran-2- | | | | |
| | carboxamide | | | | |
| 404 | (2R,3S,4S,5R)-3-(3,4-difluoro-2- | C | 528.511 | 529 | 0.97 |
| | methoxy-phenyl)-4,5-dimethyl-N-[6- | | | | |
| | (tetrahydropyran-4-ylmethyl)-3- | | | | |
| | pyridyl]-5- | | | | |
| | (trifluoromethyl)tetrahydrofuran-2- | | | | |
| | carboxamide | | | | |
| 405 | (2R,3S,4S,5R)-3-(3,4-difluoro-2- | A | 502.474 | 504 | 3.44 |
| | methoxy-phenyl)-N-[2-(2-hydroxy-2- | | | | |
| | methyl-propyl)-4-pyridyl]-4,5- | | | | |
| | dimethyl-5- | | | | |
| | (trifluoromethyl)tetrahydrofuran-2- | | | | |
| | carboxamide | | | | |
| 406 | (2R,3S,4S,5R)-N-[6-[1-(azetidin-1- | A | 543.526 | 544.3 | 3.46 |
| | yl)-2-methoxy-ethyl]-3-pyridyl]-3- | | | | |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 407 | (2R,3S,4S,5R)-N-[6-[1-(azetidin-1- | A | 543.526 | 544.3 | 3.44 |
| | yl)-2-methoxy-ethyl]-3-pyridyl]-3- | | | | |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 408 | (2R,3S,4S,5R)-3-(3,4-difluoro-2- | A | 488.405 | 489.5 | 3.23 |
| | methoxy-phenyl)-N-(3-hydroxy-2,3- | | | | |
| | dihydrofuro[3,2-b]pyridin-6-yl)-4,5- | | | | |
| | dimethyl-5- | | | | |
| | (trifluoromethyl)tetrahydrofuran-2- | | | | |
| | carboxamide | | | | |
| 409 | (2R,3S,4S,5R)-3-(3,4-difluoro-2- | A | 488.405 | 489.5 | 3.23 |
| | methoxy-phenyl)-N-(3-hydroxy-2,3- | | | | |
| | dihydrofuro[3,2-b]pyridin-6-yl)-4,5- | | | | |
| | dimethyl-5- | | | | |
| | (trifluoromethyl)tetrahydrofuran-2- | | | | |
| | carboxamide | | | | |
| 410 | (2R,3S,4S,5R)-3-(3,4-difluoro-2- | A | 545.542 | | 3.57 |
| | methoxy-phenyl)-N-[2-[[(2-methoxy- | | | | |
| | 1,1-dimethyl-ethyl)amino]methyl]-4- | | | | |
| | pyridyl]-4,5-dimethyl-5- | | | | |
| | (trifluoromethyl)tetrahydrofuran-2- | | | | |
| | carboxamide | | | | |
| 411 | (2R,3S,4S,5R)-3-(3,4-difluoro-2- | A | 528.472 | 529 | 3.03 |
| | methoxy-phenyl)-4,5-dimethyl-N-[2- | | | | |
| | (2-oxopiperazin-1-yl)-4-pyridyl]-5- | | | | |
| | (trifluoromethyl)tetrahydrofuran-2- | | | | |
| | carboxamide | | | | |
| 412 | (2R,3S,4S,5R)-3-(3,4-difluoro-2- | E | 517.489 | 518.4 | 2.88 |
| | methoxy-phenyl)-N-[2-[2-methoxy-1- | | | | |
| | (methylamino)ethyl]-4-pyridyl]-4,5- | | | | |
| | dimethyl-5- | | | | |
| | (trifluoromethyl)tetrahydrofuran-2- | | | | |
| | carboxamide | | | | |
| 413 | (2R,3S,4S,5R)-3-(3,4-difluoro-2- | E | 517.489 | 518.4 | 2.89 |
| | methoxy-phenyl)-N-[2-[2-methoxy-1- | | | | |
| | (methylamino)ethyl]-4-pyridyl]-4,5- | | | | |
| | dimethyl-5- | | | | |
| | (trifluoromethyl)tetrahydrofuran-2- | | | | |
| | carboxamide | | | | |
| 414 | (2R,3S,4S,5R)-3-(3,4-difluoro-2- | E | 517.489 | 518.3 | 2.88 |
| | methoxy-phenyl)-N-[6-[2-methoxy-1- | | | | |
| | (methylamino)ethyl]-3-pyridyl]-4,5- | | | | |
| | dimethyl-5- | | | | |
| | (trifluoromethyl)tetrahydrofuran-2- | | | | |
| | carboxamide | | | | |
| 415 | (2R,3S,4S,5R)-3-(3,4-difluoro-2- | E | 517.489 | 518.3 | 2.88 |
| | methoxy-phenyl)-N-[6-[2-methoxy-1- | | | | |
| | (methylamino)ethyl]-3-pyridyl]-4,5- | | | | |
| | dimethyl-5- | | | | |
| | (trifluoromethyl)tetrahydrofuran-2- | | | | |
| | carboxamide | | | | |
| 416 | (2R,3S,4S,5R)-3-(3,4-difluoro-2- | E | 561.541 | 562.3 | 2.97 |
| | methoxy-phenyl)-N-[6-[2-methoxy-1- | | | | |
| | (2-methoxyethylamino)ethyl]-3- | | | | |
| | pyridyl]-4,5-dimethyl-5- | | | | |
| | (trifluoromethyl)tetrahydrofuran-2- | | | | |
| | carboxamide | | | | |
| 417 | (2R,3S,4S,5R)-3-(3,4-difluoro-2- | E | 561.541 | 562 | 2.92 |
| | methoxy-phenyl)-N-[6-[2-methoxy-1- | | | | |
| | (2-methoxyethylamino)ethyl]-3- | | | | |
| | pyridyl]-4,5-dimethyl-5- | | | | |
| | (trifluoromethyl)tetrahydrofuran-2- | | | | |
| | carboxamide | | | | |
| 418 | (2R,3S,4S,5R)-3-(3,4-difluoro-2- | A [2], A | 535.479 | | 3.535 |
| | methoxy-phenyl)-N-[5-fluoro-2-[2- | | | | |
| | methoxy-1-(methylamino)ethyl]-4- | | | | |
| | pyridyl]-4,5-dimethyl-5- | | | | |
| | (trifluoromethyl)tetrahydrofuran-2- | | | | |
| | carboxamide | | | | |
| 419 | (2R,3S,4S,5R)-3-(3,4-difluoro-2- | E [2], A | 535.479 | 536.2 | 3.23 |
| | methoxy-phenyl)-N-[5-fluoro-2-[2- | | | | |
| | methoxy-1-(methylamino)ethyl]-4- | | | | |
| | pyridyl]-4,5-dimethyl-5- | | | | |
| | (trifluoromethyl)tetrahydrofuran-2- | | | | |
| | carboxamide | | | | |
| 420 | (2R,3S,4S,5R)-3-(3,4-difluoro-2- | A | 545.542 | 546.2 | 3.54 |
| | methoxy-phenyl)-N-[6-[1- | | | | |
| | (isopropylamino)-2-methoxy-ethyl]-3- | | | | |
| | pyridyl]-4,5-dimethyl-5- | | | | |
| | (trifluoromethyl)tetrahydrofuran-2- | | | | |
| | carboxamide | | | | |
| 421 | (2R,3S,4S,5R)-3-(3,4-difluoro-2- | A | 545.542 | | 3.55 |
| | methoxy-phenyl)-N-[6-[1- | | | | |
| | (isopropylamino)-2-methoxy-ethyl]-3- | | | | |
| | pyridyl]-4,5-dimethyl-5- | | | | |
| | (trifluoromethyl)tetrahydrofuran-2- | | | | |
| | carboxamide | | | | |
| 422 | (2R,3S,4S,5R)-N-[2-(1-amino-2- | A | 521.453 | 522.6 | 3.4 |
| | methoxy-ethyl)-5-fluoro-4-pyridyl]-3- | | | | |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 423 | (2R,3S,4S,5R)-N-[2-(1-amino-2- | A | 521.453 | 522.6 | 3.39 |
| | methoxy-ethyl)-5-fluoro-4-pyridyl]-3- | | | | |
| | (3,4-difluoro-2-methoxy-phenyl)-4,5- | | | | |
| | dimethyl-5- | | | | |
| | (trifluoromethyl)tetrahydrofuran-2- | | | | |
| | carboxamide | | | | |
| 424 | (2R,3S,4S,5R)-3-(3,4-difluoro-2- | E | 531.515 | 532.4 | 2.92 |
| | methoxy-phenyl)-N-[2-[1- | | | | |
| | (dimethylamino)-2-methoxy-ethyl]-4- | | | | |
| | pyridyl]-4,5-dimethyl-5- | | | | |
| | (trifluoromethyl)tetrahydrofuran-2- | | | | |
| | carboxamide | | | | |
| 425 | (2R,3S,4S,5R)-3-(3,4-difluoro-2- | E | 531.515 | 532.4 | 2.92 |
| | methoxy-phenyl)-N-[2-[1- | | | | |
| | (dimethylamino)-2-methoxy-ethyl]-4- | | | | |
| | pyridyl]-4,5-dimethyl-5- | | | | |
| | (trifluoromethyl)tetrahydrofuran-2- | | | | |
| | carboxamide | | | | |
| 426 | (2R,3S,4S,5R)-3-(3,4-difluoro-2- | A | 531.515 | | 3.35 |
| | methoxy-phenyl)-N-[6-[1- | | | | |
| | (dimethylamino)-2-methoxy-ethyl]-3- | | | | |
| | pyridyl]-4,5-dimethyl-5- | | | | |
| | (trifluoromethyl)tetrahydrofuran-2- | | | | |
| | carboxamide | | | | |
| 427 | (2R,3S,4S,5R)-3-(3,4-difluoro-2- | E | 531.515 | 532.3 | 2.92 |
| | methoxy-phenyl)-N-[6-[1- | | | | |
| | (dimethylamino)-2-methoxy-ethyl]-3- | | | | |
| | pyridyl]-4,5-dimethyl-5- | | | | |
| | (trifluoromethyl)tetrahydrofuran-2- | | | | |
| | carboxamide | | | | |
| 428 | (2R,3S,4S,5R)-N-[2-(1-amino-2- | A | 535.479 | 537.2 | 3.36 |
| | hydroxy-2-methyl-propyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 429 | (2R,3S,4S,5R)-N-[2-(1-amino-2- | G | 535.479 | 537.1 | 3.37 |
| | hydroxy-2-methyl-propyl)-5-fluoro-4- | | | | |
| | pyridyl]-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 430 | (2R,3S,4S,5R)-3-(3,4-difluoro-2- | A | 530.445 | 531.6 | 3.12 |
| | methoxy-phenyl)-N-[2-(2- | | | | |
| | hydroxyethyl)-3-oxo- | | | | |
| | [1,2,4]triazolo[4,3-a]pyridin-7-yl]-4,5- | | | | |
| | dimethyl-5- | | | | |
| | (trifluoromethyl)tetrahydrofuran-2- | | | | |
| | carboxamide | | | | |
| 431 | (2R,3S,4S,5R)-3-(3,4-difluoro-2- | A | 500.419 | 501.5 | 3.04 |
| | methoxy-phenyl)-N-[3- | | | | |
| | (hydroxymethyl)-[1,2,4]triazolo[4,3- | | | | |
| | alpyridin-7-yl]-4,5-dimethyl-5- | | | | |
| | (trifluoromethyl)tetrahydrofuran-2- | | | | |
| | carboxamide | | | | |
| 432 | (2R,3S,4S,5R)-3-(3,4-difluoro-2- | A | 500.419 | 501.5 | 3.07 |
| | methoxy-phenyl)-N-[3- | | | | |
| | (hydroxymethyl)-[1,2,4]triazolo[4,3- | | | | |
| | alpyridin-6-yl]-4,5-dimethyl-5- | | | | |
| | (trifluoromethyl)tetrahydrofuran-2- | | | | |
| | carboxamide | | | | |
| 433 | (2R,3S,4S,5R)-3-(3,4-difluoro-2- | A | 486.432 | 487.6 | 3.26 |
| | methoxy-phenyl)-N-(7-hydroxy-6,7- | | | | |
| | dihydro-5H-cyclopenta[b]pyridin-3- | | | | |
| | yl)-4,5-dimethyl-5- | | | | |
| | (trifluoromethyl)tetrahydrofuran-2- | | | | |
| | carboxamide | | | | |
| 434 | (2R,3S,4S,5R)-3-(3,4-difluoro-2- | A | 486.432 | 487.6 | 3.14 |
| | methoxy-phenyl)-N-(5-hydroxy-6,7- | | | | |
| | dihydro-5H-cyclopenta[b]pyridin-3- | | | | |
| | yl)-4,5-dimethyl-5- | | | | |
| | (trifluoromethyl)tetrahydrofuran-2- | | | | |
| | carboxamide | | | | |
| 435 | (2R,3S,4S,5R)-3-(3,4-difluoro-2- | A | 486.432 | 487.5 | 3.14 |
| | methoxy-phenyl)-N-(5-hydroxy-6,7- | | | | |
| | dihydro-5H-cyclopenta[b]pyridin-3- | | | | |
| | yl)-4,5-dimethyl-5- | | | | |
| | (trifluoromethyl)tetrahydrofuran-2- | | | | |
| | carboxamide | | | | |
| 436 | (2R,3S,4S,5R)-3-(3,4-difluoro-2- | A | 544.511 | 546 | 3.73 |
| | methoxy-phenyl)-N-[2-(2- | | | | |
| | hydroxycyclohexoxy)-4-pyridyl]-4,5- | | | | |
| | dimethyl-5- | | | | |
| | (trifluoromethyl)tetrahydrofuran-2- | | | | |
| | carboxamide | | | | |
| 437 | (2R,3S,4S,5R)-3-(3,4-difluoro-2- | A | 532.457 | 533 | 3.16 |
| | methoxy-phenyl)-N-[5-(4- | | | | |
| | hydroxytetrahydrofuran-3-yl)oxy-3- | | | | |
| | pyridyl]-4,5-dimethyl-5- | | | | |
| | (trifluoromethyl)tetrahydrofuran-2- | | | | |
| | carboxamide | | | | |
| 438 | (2R,3S,4S,5R)-3-(3,4-difluoro-2- | A | 492.411 | 494 | 3.28 |
| | methoxy-phenyl)-N-[6-(1-fluoro-2- | | | | |
| | hydroxy-ethyl)-3-pyridyl]-4,5- | | | | |
| | dimethyl-5- | | | | |
| | (trifluoromethyl)tetrahydrofuran-2- | | | | |
| | carboxamide | | | | |
| 439 | (2R,3S,4S,5R)-3-(3,4-difluoro-2- | A | 492.411 | 493.3 | 3.3 |
| | methoxy-phenyl)-N-[2-(1-fluoro-2- | | | | |
| | hydroxy-ethyl)-4-pyridyl]-4,5- | | | | |
| | dimethyl-5- | | | | |
| | (trifluoromethyl)tetrahydrofuran-2- | | | | |
| | carboxamide | | | | |
| 440 | (2R,3S,4S,5R)-3-(3,4-difluoro-2- | A | 492.411 | | 3.31 |
| | methoxy-phenyl)-N-[2-(1-fluoro-2- | | | | |
| | hydroxy-ethyl)-4-pyridyl]-4,5- | | | | |
| | dimethyl-5- | | | | |
| | (trifluoromethyl)tetrahydrofuran-2- | | | | |
| | carboxamide | | | | |
| 441 | 4-((2R,3S,4S,5R)-3-(3,4-difluoro-2- | LCMS | 508.411 | 509.2 | 3.03 |
| | methoxyphenyl)-4,5-dimethyl-5- | | | | |
| | (trifluoromethyl)tetrahydrofuran-2- | | | | |
| | carboxamido)-2-(1-fluoro-2- | | | | |
| | hydroxyethyl)pyridine 1-oxide | | | | |
| 442 | (2R,3S,4S,5R)-3-(3,4-difluoro-2- | A | 500.419 | 501.2 | 3.1 |
| | methoxy-phenyl)-N-[2- | | | | |
| | (hydroxymethyl)-[1,2,4]triazolo[1,5- | | | | |
| | alpyridin-6-yl]-4,5-dimethyl-5- | | | | |
| | (trifluoromethyl)tetrahydrofuran-2- | | | | |
| | carboxamide | | | | |
| 443 | (2R,3S,4S,5R)-3-(3,4-difluoro-2- | A | 500.419 | 501.3 | 3.11 |
| | methoxy-phenyl)-N-[2- | | | | |
| | (hydroxymethyl)-[1,2,4]triazolo[1,5- | | | | |
| | alpyridin-7-yl]-4,5-dimethyl-5- | | | | |
| | (trifluoromethyl)tetrahydrofuran-2- | | | | |
| | carboxamide | | | | |
| 444 | (2R,3S,4S,5R)-3-(3,4-difluoro-2- | A | 447.356 | 448.3 | 2.53 |
| | methoxy-phenyl)-N-(5- | | | | |
| | hydroxypyrimidin-2-yl)-4,5-dimethyl- | | | | |
| | 5-(trifluoromethyl)tetrahydrofuran-2- | | | | |
| | carboxamide | | | | |
| 445 | (2R,3S,4S,5R)-3-(3,4-difluoro-2- | A | 477.425 | 478 | 2.91 |
| | methoxy-phenyl)-N-[2-(2- | | | | |
| | hydroxyethyl)-3-methyl-imidazol-4- | | | | |
| | yl]-4,5-dimethyl-5- | | | | |
| | (trifluoromethyl)tetrahydrofuran-2- | | | | |
| | carboxamide | | | | |
| 446 | (2R,3S,4S,5R)-3-(3,4-difluoro-2- | A | 493.4 | 494.6 | 3.17 |
| | methoxy-phenyl)-N-[2-(1-fluoro-2- | | | | |
| | hydroxy-ethyl)pyrimidin-5-yl]-4,5- | | | | |
| | dimethyl-5- | | | | |
| | (trifluoromethyl)tetrahydrofuran-2- | | | | |
| | carboxamide | | | | |
| 447 | (2R,3S,4S,5R)-3-(3,4-difluoro-2- | A | 447.399 | 448 | 3.09 |
| | methoxy-phenyl)-N-(2,3- | | | | |
| | dimethylimidazol-4-yl)-4,5-dimethyl- | | | | |
| | 5-(trifluoromethyl)tetrahydrofuran-2- | | | | |
| | carboxamide | | | | |
| 448 | 3-(3,4-difluoro-2-methoxy-phenyl)-N- | A | 537.5 | 538.1 | 3.52 |
| | [2-(dimethylsulfamoyl)-4-pyridyl]- | | | | |
| | 4,5-dimethyl-5- | | | | |
| | (trifluoromethyl)tetrahydrofuran-2- | | | | |
| | carboxamide | | | | |
| 449 | 3-(3,4-difluoro-2-methoxy-phenyl)-N- | A | 537.5 | 538.1 | 3.52 |
| | [2-(dimethylsulfamoyl)-4-pyridyl]- | | | | |
| | 4,5-dimethyl-5- | | | | |
| | (trifluoromethyl)tetrahydrofuran-2- | | | | |
| | carboxamide | | | | |
| 450 | 3-(3,4-difluoro-2-methoxy-phenyl)- | A | 471.381 | 472.6 | 3.32 |
| | 4,5-dimethyl-N-(tetrazolo[1,5- | | | | |
| | alpyridin-6-yl)-5- | | | | |
| | (trifluoromethyl)tetrahydrofuran-2- | | | | |
| | carboxamide | | | | |
| 451 | 3-(3,4-difluoro-2-methoxy-phenyl)- | A | 471.381 | 472.6 | 3.31 |
| | 4,5-dimethyl-N-(tetrazolo[1,5- | | | | |
| | alpyridin-6-yl)-5- | | | | |
| | (trifluoromethyl)tetrahydrofuran-2- | | | | |
| | carboxamide | | | | |
| 452 | 3-(3,4-difluoro-2-methoxy-phenyl)-N- | A | 537.5 | 538.1 | 3.45 |
| | [5-(dimethylsulfamoyl)-3-pyridyl]- | | | | |
| | 4,5-dimethyl-5- | | | | |
| | (trifluoromethyl)tetrahydrofuran-2- | | | | |
| | carboxamide | | | | |
| 453 | 3-(3,4-difluoro-2-methoxy-phenyl)-N- | A | 537.5 | 538.1 | 3.45 |
| | [5-(dimethylsulfamoyl)-3-pyridyl]- | | | | |
| | 4,5-dimethyl-5- | | | | |
| | (trifluoromethyl)tetrahydrofuran-2- | | | | |
| | carboxamide | | | | |
| 454 | 3-(3,4-difluoro-2-methoxy-phenyl)- | A | 471.381 | 472.6 | 3.3 |
| | 4,5-dimethyl-N-(tetrazolo[1,5- | | | | |
| | alpyridin-6-yl)-5- | | | | |
| | (trifluoromethyl)tetrahydrofuran-2- | | | | |
| | carboxamide | | | | |
| 455 | 3-(3,4-difluoro-2-methoxy-phenyl)- | A | 523.473 | 524.5 | 3.38 |
| | 4,5-dimethyl-N-[2-(methylsulfamoyl)- | | | | |
| | 4-pyridyl]-5- | | | | |
| | (trifluoromethyl)tetrahydrofuran-2- | | | | |
| | carboxamide | | | | |
| 456 | 3-(3,4-difluoro-2-methoxy-phenyl)- | A | 523.473 | 524.5 | 3.37 |
| | 4,5-dimethyl-N-[2-(methylsulfamoyl)- | | | | |
| | 4-pyridyl]-5- | | | | |
| | (trifluoromethyl)tetrahydrofuran-2- | | | | |
| | carboxamide | | | | |
| 457 | 3-[2-(difluoromethoxy)-3,4-difluoro- | A | 510.402 | 511.6 | 3.19 |
| | phenyl]-N-[2-(2-hydroxyethyl)-4- | | | | |
| | pyridyl]-4,5-dimethyl-5- | | | | |
| | (trifluoromethyl)tetrahydrofuran-2- | | | | |
| | carboxamide | | | | |
| 458 | 3-[2-(difluoromethoxy)-3,4-difluoro- | A | 510.402 | 511.6 | 3.19 |
| | phenyl]-N-[2-(2-hydroxyethyl)-4- | | | | |
| | pyridyl]-4,5-dimethyl-5- | | | | |
| | (trifluoromethyl)tetrahydrofuran-2- | | | | |
| | carboxamide | | | | |
| 459 | 3-[2-(difluoromethoxy)-3,4-difluoro- | A | 510.402 | 511.1 | 3.12 |
| | phenyl]-N-[6-(2-hydroxyethyl)-3- | | | | |
| | pyridyl]-4,5-dimethyl-5- | | | | |
| | (trifluoromethyl)tetrahydrofuran-2- | | | | |
| | carboxamide | | | | |
| 460 | 3-[2-(difluoromethoxy)-3,4-difluoro- | A | 510.402 | 511.1 | 3.13 |
| | phenyl]-N-[6-(2-hydroxyethyl)-3- | | | | |
| | pyridyl]-4,5-dimethyl-5- | | | | |
| | (trifluoromethyl)tetrahydrofuran-2- | | | | |
| | carboxamide | | | | |
| 461 | 3-[2-(difluoromethoxy)-3,4-difluoro- | A | 538.455 | 539.6 | 3.35 |
| | phenyl]-N-[6-(2-hydroxy-2-methyl- | | | | |
| | propyl)-3-pyridyl]-4,5-dimethyl-5- | | | | |
| | (trifluoromethyl)tetrahydrofuran-2- | | | | |
| | carboxamide | | | | |
| 462 | 3-[2-(difluoromethoxy)-3,4-difluoro- | A | 538.455 | 539.6 | 3.35 |
| | phenyl]-N-[6-(2-hydroxy-2-methyl- | | | | |
| | propyl)-3-pyridyl]-4,5-dimethyl-5- | | | | |
| | (trifluoromethyl)tetrahydrofuran-2- | | | | |
| | carboxamide | | | | |
| 463 | 3-[2-(difluoromethoxy)-3,4-difluoro- | A | 538.455 | 539.7 | 3.39 |
| | phenyl]-N-[2-(2-hydroxy-2-methyl- | | | | |
| | propyl)-4-pyridyl]-4,5-dimethyl-5- | | | | |
| | (trifluoromethyl)tetrahydrofuran-2- | | | | |
| | carboxamide | | | | |
| 464 | 3-[2-(difluoromethoxy)-3,4-difluoro- | A | 538.455 | 539.6 | 3.4 |
| | phenyl]-N-[2-(2-hydroxy-2-methyl- | | | | |
| | propyl)-4-pyridyl]-4,5-dimethyl-5- | | | | |
| | (trifluoromethyl)tetrahydrofuran-2- | | | | |
| | carboxamide | | | | |
| 465 | (2R,3S,4S,5R)-3-(3,4-difluoro-2- | A [3], F | 556.525 | 557.14 | 2.79 |
| | methoxy-phenyl)-N-[2-[(2R)-2,4- | [2], F | | | |
| | dimethyl-6-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 466 | (2R,3S,4S,5R)-3-(3,4-difluoro-2- | A | 556.525 | 556.9 | 3.19 |
| | methoxy-phenyl)-4,5-dimethyl-N-[2- | | | | |
| | (4-methyl-7-oxo-1,4-diazepan-1-yl)-4- | | | | |
| | pyridyl]-5- | | | | |
| | (trifluoromethyl)tetrahydrofuran-2- | | | | |
| | carboxamide | | | | |
| 467 | (2R,3S,4S,5R)-3-(3,4-difluoro-2- | F [2], F | 556.525 | 557.16 | 2.49 |
| | methoxy-phenyl)-4,5-dimethyl-N-[2- | | | | |
| | (4-methyl-2-oxo-1,4-diazepan-1-yl)-4- | | | | |
| | pyridyl]-5- | | | | |
| | (trifluoromethyl)tetrahydrofuran-2- | | | | |
| | carboxamide | | | | |
| 468 | (2R,3S,4S,5R)-3-(3,4-difluoro-2- | A | 568.536 | 568.9 | 3.15 |
| | methoxy-phenyl)-4,5-dimethyl-N-[2- | | | | |
| | (3-oxo-1,4-diazabicyclo[3.2.2]nonan- | | | | |
| | 4-yl)-4-pyridyl]-5- | | | | |
| | (trifluoromethyl)tetrahydrofuran-2- | | | | |
| | carboxamide | | | | |
| 469 | (2R,3S,4S,5R)-3-(3,4-difluoro-2- | F | 556.525 | 557.2 | 2.54 |
| | methoxy-phenyl)-N-[2-[(2S)-2,4- | | | | |
| | dimethyl-6-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 470 | (2R,3S,4S,5R)-3-(3,4-difluoro-2- | F | 556.525 | 557.19 | 2.56 |
| | methoxy-phenyl)-N-[2-[(5R)-4,5- | | | | |
| | dimethyl-2-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 471 | (2R,3S,4S,5R)-3-(3,4-difluoro-2- | F | 529.456 | 530.13 | 2.45 |
| | methoxy-phenyl)-N-[2-[(3R)-3- | | | | |
| | hydroxy-2-oxo-pyrrolidin-1-yl]-4- | | | | |
| | pyridyl]-4,5-dimethyl-5- | | | | |
| | (trifluoromethyl)tetrahydrofuran-2- | | | | |
| | carboxamide | | | | |
| 472 | (2R,3S,4S,5R)-3-(3,4-difluoro-2- | F | 530.488 | 531.17 | 2.75 |
| | methoxy-phenyl)-N-[2-[[2- | | | | |
| | (dimethylamino)acetyl]amino]-4- | | | | |
| | pyridyl]-4,5-dimethyl-5- | | | | |
| | (trifluoromethyl)tetrahydrofuran-2- | | | | |
| | carboxamide | | | | |
| 473 | (2R,3S,4S,5R)-3-(3,4-difluoro-2- | F | 529.456 | 530.16 | 2.45 |
| | methoxy-phenyl)-N-[2-[(3S)-3- | | | | |
| | hydroxy-2-oxo-pyrrolidin-1-yl]-4- | | | | |
| | pyridyl]-4,5-dimethyl-5- | | | | |
| | (trifluoromethyl)tetrahydrofuran-2- | | | | |
| | carboxamide | | | | |
| 474 | (2R,3S,4S,5R)-3-(3,4-difluoro-2- | F | 556.525 | 557.18 | 2.49 |
| | methoxy-phenyl)-N-[2-[(5S)-4,5- | | | | |
| | dimethyl-2-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 475 | (2R,3S,4S,5R)-3-(3,4-difluoro-2- | F | 556.525 | 557.18 | 2.5 |
| | methoxy-phenyl)-N-[2-[(3R)-3,4- | | | | |
| | dimethyl-2-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 476 | (2R,3S,4S,5R)-3-(3,4-difluoro-2- | F | 556.525 | 557.23 | 2.58 |
| | methoxy-phenyl)-N-[2-[(3S)-3,4- | | | | |
| | dimethyl-2-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 477 | (2R,3S,4S,5R)-3-(3,4-difluoro-2- | F [2], F | 542.498 | 543.125 | 2.305 |
| | methoxy-phenyl)-4,5-dimethyl-N-[2- | | | | |
| | [(2R)-2-methyl-6-oxo-piperazin-1-yl]- | | | | |
| | 4-pyridyl]-5- | | | | |
| | (trifluoromethyl)tetrahydrofuran-2- | | | | |
| | carboxamide | | | | |
| 478 | (2R,3S,4S,5R)-3-(3,4-difluoro-2- | F | 542.498 | 543.22 | 2.37 |
| | methoxy-phenyl)-4,5-dimethyl-N-[2- | | | | |
| | [(5S)-5-methyl-2-oxo-piperazin-1-yl]- | | | | |
| | 4-pyridyl]-5- | | | | |
| | (trifluoromethyl)tetrahydrofuran-2- | | | | |
| | carboxamide | | | | |
| 479 | (2R,3S,4S,5R)-3-(3,4-difluoro-2- | F | 542.498 | 543.14 | 2.34 |
| | methoxy-phenyl)-4,5-dimethyl-N-[2- | | | | |
| | [(2S)-2-methyl-6-oxo-piperazin-1-yl]- | | | | |
| | 4-pyridyl]-5- | | | | |
| | (trifluoromethyl)tetrahydrofuran-2- | | | | |
| | carboxamide | | | | |
| 480 | (2R,3S,4S,5R)-3-(3,4-difluoro-2- | F | 542.498 | 543.14 | 2.37 |
| | methoxy-phenyl)-4,5-dimethyl-N-[2- | | | | |
| | [(5R)-5-methyl-2-oxo-piperazin-1-yl]- | | | | |
| | 4-pyridyl]-5- | | | | |
| | (trifluoromethyl)tetrahydrofuran-2- | | | | |
| | carboxamide | | | | |
| 481 | (2R,3S,4S,5R)-3-(3,4-difluoro-2- | F | 542.498 | 543.21 | 2.37 |
| | methoxy-phenyl)-4,5-dimethyl-N-[2- | | | | |
| | (2-oxo-1,4-diazepan-1-yl)-4-pyridyl]- | | | | |
| | 5-(trifluoromethyl)tetrahydrofuran-2- | | | | |
| | carboxamide | | | | |
| 482 | (2R,3S,4S,5R)-3-(3,4-difluoro-2- | F | 540.482 | 541.1 | 2.46 |
| | methoxy-phenyl)-4,5-dimethyl-N-[2- | | | | |
| | [(1R,4R)-3-oxo-2,5- | | | | |
| | diazabicyclo[2.2.1]heptan-2-yl]-4- | | | | |
| | pyridyl]-5- | | | | |
| | (trifluoromethyl)tetrahydrofuran-2- | | | | |
| | carboxamide | | | | |
| 483 | (2R,3S,4S,5R)-3-(3,4-difluoro-2- | F | 540.482 | 541.2 | 2.46 |
| | methoxy-phenyl)-4,5-dimethyl-N-[2- | | | | |
| | [(1S,4S)-3-oxo-2,5- | | | | |
| | diazabicyclo[2.2.1]heptan-2-yl]-4- | | | | |
| | pyridyl]-5- | | | | |
| | (trifluoromethyl)tetrahydrofuran-2- | | | | |
| | carboxamide | | | | |
| 484 | (2R,3S,4S,5R)-3-(3,4-difluoro-2- | F | 542.498 | 543.22 | 2.29 |
| | methoxy-phenyl)-4,5-dimethyl-N-[2- | | | | |
| | [(3R)-3-methyl-2-oxo-piperazin-1-yl]- | | | | |
| | 4-pyridyl]-5- | | | | |
| | (trifluoromethyl)tetrahydrofuran-2- | | | | |
| | carboxamide | | | | |
| 485 | (2R,3S,4S,5R)-3-(3,4-difluoro-2- | F | 542.498 | 543.18 | 2.27 |
| | methoxy-phenyl)-4,5-dimethyl-N-[2- | | | | |
| | [(3S)-3-methyl-2-oxo-piperazin-1-yl]- | | | | |
| | 4-pyridyl]-5- | | | | |
| | (trifluoromethyl)tetrahydrofuran-2- | | | | |
| | carboxamide | | | | |
| 486 | (2R,3S,4S,5R)-3-(3,4-difluoro-2- | F | 530.488 | 531.17 | 2.32 |
| | methoxy-phenyl)-4,5-dimethyl-N-[2- | | | | |
| | [methyl-[2- | | | | |
| | (methylamino)acetyl]amino]-4- | | | | |
| | pyridyl]-5- | | | | |
| | (trifluoromethyl)tetrahydrofuran-2- | | | | |
| | carboxamide | | | | |
| 487 | (2R,3S,4S,5R)-3-(3,4-difluoro-2- | F | 530.488 | 531.2 | 2.41 |
| | methoxy-phenyl)-4,5-dimethyl-N-[2- | | | | |
| | [methyl-[2-(methylamino)-2-oxo- | | | | |
| | ethyl]amino]-4-pyridyl]-5- | | | | |
| | (trifluoromethyl)tetrahydrofuran-2- | | | | |
| | carboxamide | | | | |
| 488 | (2R,3S,4S,5R)-3-(3,4-difluoro-2- | F | 516.461 | 517.21 | 2.45 |
| | methoxy-phenyl)-4,5-dimethyl-N-[2- | | | | |
| | [[2-(methylamino)acetyl]amino]-4- | | | | |
| | pyridyl]-5- | | | | |
| | (trifluoromethyl)tetrahydrofuran-2- | | | | |
| | carboxamide | | | | |
| 489 | (2R,3S,4S,5R)-3-(3,4-difluoro-2- | F | 554.509 | 555.2 | 2.56 |
| | methoxy-phenyl)-4,5-dimethyl-N-[2- | | | | |
| | [(1S,4S)-5-methyl-3-oxo-2,5- | | | | |
| | diazabicyclo[2.2.1]heptan-2-yl]-4- | | | | |
| | pyridyl]-5- | | | | |
| | (trifluoromethyl)tetrahydrofuran-2- | | | | |
| | carboxamide | | | | |
| 490 | (2R,3S,4S,5R)-3-(3,4-difluoro-2- | F | 554.509 | 555.2 | 2.6 |
| | methoxy-phenyl)-4,5-dimethyl-N-[2- | | | | |
| | [(1R,4R)-5-methyl-3-oxo-2,5- | | | | |
| | diazabicyclo[2.2.1]heptan-2-yl]-4- | | | | |
| | pyridyl]-5- | | | | |
| | (trifluoromethyl)tetrahydrofuran-2- | | | | |
| | carboxamide | | | | |
| 491 | (2R,3S,4S,5R)-3-(3,4-difluoro-2- | A | 540.482 | 541 | 3.2 |
| | methoxy-phenyl)-4,5-dimethyl-N-[2- | | | | |
| | (2-oxo-3,6-diazabicyclo[3.1.1]heptan- | | | | |
| | 3-yl)-4-pyridyl]-5- | | | | |
| | (trifluoromethyl)tetrahydrofuran-2- | | | | |
| | carboxamide | | | | |
| 492 | (2R,3S,4S,5R)-3-(3,4-difluoro-2- | A | 540.482 | 541 | 3.21 |
| | methoxy-phenyl)-4,5-dimethyl-N-[2- | | | | |
| | (2-oxo-3,6-diazabicyclo[3.1.1]heptan- | | | | |
| | 3-yl)-4-pyridyl]-5- | | | | |
| | (trifluoromethyl)tetrahydrofuran-2- | | | | |
| | carboxamide | | | | |
| 493 | (2R,3S,4S,5R)-3-(3,4-difluoro-2- | A | 554.509 | 555 | 3.17 |
| | methoxy-phenyl)-4,5-dimethyl-N-[2- | | | | |
| | (2-oxo-3,8-diazabicyclo[3.2.1]octan- | | | | |
| | 3-yl)-4-pyridyl]-5- | | | | |
| | (trifluoromethyl)tetrahydrofuran-2- | | | | |
| | carboxamide | | | | |
| 494 | (2R,3S,4S,5R)-3-(3,4-difluoro-2- | A | 554.509 | 555 | 3.17 |
| | methoxy-phenyl)-4,5-dimethyl-N-[2- | | | | |
| | (2-oxo-3,8-diazabicyclo[3.2.1]octan- | | | | |
| | 3-yl)-4-pyridyl]-5- | | | | |
| | (trifluoromethyl)tetrahydrofuran-2- | | | | |
| | carboxamide | | | | |
| 495 | (2R,3S,4S,5R)-N-[2-(4-amino-2-oxo- | A | 528.472 | 529 | 3.13 |
| | pyrrolidin-1-yl)-4-pyridyl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 496 | (2R,3S,4S,5R)-N-[2-(4-amino-2-oxo- | A | 528.472 | 529 | 3.13 |
| | pyrrolidin-1-yl)-4-pyridyl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 497 | (2R,3S,4S,5R)-N-[2-(3-amino-2-oxo- | A | 528.472 | 529 | 3.18 |
| | pyrrolidin-1-yl)-4-pyridyl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 498 | (2R,3S,4S,5R)-N-[2-(3-amino-2-oxo- | A | 528.472 | 529 | 3.18 |
| | pyrrolidin-1-yl)-4-pyridyl]-3-(3,4- | | | | |
| | difluoro-2-methoxy-phenyl)-4,5- | | | | |
| | dimethyl-5- | | | | |
| | (trifluoromethyl)tetrahydrofuran-2- | | | | |
| | carboxamide | | | | |
| 499 | (2R,3S,4S,5R)-3-(3,4-difluoro-2- | A | 542.498 | 543 | 3.23 |
| | methoxy-phenyl)-4,5-dimethyl-N-[2- | | | | |
| | [4-(methylamino)-2-oxo-pyrrolidin-1- | | | | |
| | yl]-4-pyridyl]-5- | | | | |
| | (trifluoromethyl)tetrahydrofuran-2- | | | | |
| | carboxamide | | | | |
| 500 | (2R,3S,4S,5R)-3-(3,4-difluoro-2- | A | 542.498 | 543 | 3.23 |
| | methoxy-phenyl)-4,5-dimethyl-N-[2- | | | | |
| | [4-(methylamino)-2-oxo-pyrrolidin-1- | | | | |
| | yl]-4-pyridyl]-5- | | | | |
| | (trifluoromethyl)tetrahydrofuran-2- | | | | |
| | carboxamide | | | | |
| 501 | (2R,3S,4S,5R)-3-(3,4-difluoro-2- | A | 542.498 | 543 | 3.31 |
| | methoxy-phenyl)-4,5-dimethyl-N-[2- | | | | |
| | [3-(methylamino)-2-oxo-pyrrolidin-1- | | | | |
| | yl]-4-pyridyl]-5- | | | | |
| | (trifluoromethyl)tetrahydrofuran-2- | | | | |
| | carboxamide | | | | |
| 502 | (2R,3S,4S,5R)-3-(3,4-difluoro-2- | A | 542.498 | 543 | 3.31 |
| | methoxy-phenyl)-4,5-dimethyl-N-[2- | | | | |
| | [3-(methylamino)-2-oxo-pyrrolidin-1- | | | | |
| | yl]-4-pyridyl]-5- | | | | |
| | (trifluoromethyl)tetrahydrofuran-2- | | | | |
| | carboxamide | | | | |
| 503 | (2R,3S,4S,5R)-3-(3,4-difluoro-2- | A | 568.536 | 569 | 3.29 |
| | methoxy-phenyl)-4,5-dimethyl-N-[2- | | | | |
| | (8-methyl-2-oxo-3,8- | | | | |
| | diazabicyclo[3.2.1]octan-3-yl)-4- | | | | |
| | pyridyl]-5- | | | | |
| | (trifluoromethyl)tetrahydrofuran-2- | | | | |
| | carboxamide | | | | |
| 504 | (2R,3S,4S,5R)-3-(3,4-difluoro-2- | A | 568.536 | 569 | 3.29 |
| | methoxy-phenyl)-4,5-dimethyl-N-[2- | | | | |
| | (8-methyl-2-oxo-3,8- | | | | |
| | diazabicyclo[3.2.1]octan-3-yl)-4- | | | | |
| | pyridyl]-5- | | | | |
| | (trifluoromethyl)tetrahydrofuran-2- | | | | |
| | carboxamide | | | | |
| 505 | (2R,3S,4S,5R)-3-(3,4-difluoro-2- | A | 556.525 | 557 | 3.36 |
| | methoxy-phenyl)-N-[2-[4- | | | | |
| | (dimethylamino)-2-oxo-pyrrolidin-1- | | | | |
| | yl]-4-pyridyl]-4,5-dimethyl-5- | | | | |
| | (trifluoromethyl)tetrahydrofuran-2- | | | | |
| | carboxamide | | | | |
| 506 | (2R,3S,4S,5R)-3-(3,4-difluoro-2- | A | 556.525 | 557 | 3.36 |
| | methoxy-phenyl)-N-[2-[4- | | | | |
| | (dimethylamino)-2-oxo-pyrrolidin-1- | | | | |
| | yl]-4-pyridyl]-4,5-dimethyl-5- | | | | |
| | (trifluoromethyl)tetrahydrofuran-2- | | | | |
| | carboxamide | | | | |
| 507 | (2R,3S,4S,5R)-3-(3,4-difluoro-2- | A | 556.525 | 557 | 3.43 |
| | methoxy-phenyl)-N-[2-[3- | | | | |
| | (dimethylamino)-2-oxo-pyrrolidin-1- | | | | |
| | yl]-4-pyridyl]-4,5-dimethyl-5- | | | | |
| | (trifluoromethyl)tetrahydrofuran-2- | | | | |
| | carboxamide | | | | |
| 508 | (2R,3S,4S,5R)-3-(3,4-difluoro-2- | A | 556.525 | 557 | 3.42 |
| | methoxy-phenyl)-N-[2-[3- | | | | |
| | (dimethylamino)-2-oxo-pyrrolidin-1- | | | | |
| | yl]-4-pyridyl]-4,5-dimethyl-5- | | | | |
| | (trifluoromethyl)tetrahydrofuran-2- | | | | |
| | carboxamide | | | | |
| 509 | (2R,3S,4S,5R)-3-(3,4-difluoro-2- | A | 554.509 | 555 | 3.34 |
| | methoxy-phenyl)-4,5-dimethyl-N-[2- | | | | |
| | (6-methyl-2-oxo-3,6- | | | | |
| | diazabicyclo[3.1.1]heptan-3-yl)-4- | | | | |
| | pyridyl]-5- | | | | |
| | (trifluoromethyl)tetrahydrofuran-2- | | | | |
| | carboxamide | | | | |
| 510 | (2R,3S,4S,5R)-3-(3,4-difluoro-2- | A | 554.509 | 555 | 3.34 |
| | methoxy-phenyl)-4,5-dimethyl-N-[2- | | | | |
| | (6-methyl-2-oxo-3,6- | | | | |
| | diazabicyclo[3.1.1]heptan-3-yl)-4- | | | | |
| | pyridyl]-5- | | | | |
| | (trifluoromethyl)tetrahydrofuran-2- | | | | |
| | carboxamide | | | | |
| 511 | (2R,3S,4S,5R)-3-(3,4-difluoro-2- | A | 572.524 | 573 | 3.11 |
| | methoxy-phenyl)-N-[2-[(2R)-2- | | | | |
| | (hydroxymethyl)-4-methyl-6-oxo- | | | | |
| | piperazin-1-yl]-4-pyridyl]-4,5- | | | | |
| | dimethyl-5- | | | | |
| | (trifluoromethyl)tetrahydrofuran-2- | | | | |
| | carboxamide | | | | |
| 512 | (2R,3S,4S,5R)-3-(3,4-difluoro-2- | A | 572.524 | 573 | 3.11 |
| | methoxy-phenyl)-N-[2-[(2S)-2- | | | | |
| | (hydroxymethyl)-4-methyl-6-oxo- | | | | |
| | piperazin-1-yl]-4-pyridyl]-4,5- | | | | |
| | dimethyl-5- | | | | |
| | (trifluoromethyl)tetrahydrofuran-2- | | | | |
| | carboxamide | | | | |
| 513 | (2R,3S,4S,5R)-3-(3,4-difluoro-2- | F | 557.513 | 558.22 | 2.83 |
| | methoxy-phenyl)-N-[6-[(2R)-2,4- | | | | |
| | dimethyl-6-oxo-piperazin-1- | | | | |
| | yl\|pyrimidin-4-yl]-4,5-dimethyl-5- | | | | |
| | (trifluoromethyl)tetrahydrofuran-2- | | | | |
| | carboxamide | | | | |
| 514 | (2R,3S,4S,5R)-3-[2- | A | 564.453 | 565 | 3.11 |
| | (difluoromethoxy)-3,4-difluoro- | | | | |
| | phenyl]-4,5-dimethyl-N-[2-(2- | | | | |
| | oxopiperazin-1-yl)-4-pyridyl]-5- | | | | |
| | (trifluoromethyl)tetrahydrofuran-2- | | | | |
| | carboxamide | | | | |
| 515 | (2R,3S,4S,5R)-3-[3,4-difluoro-2- | A | 531.49 | 532 | 3.06 |
| | (trideuteriomethoxy)phenyl]-4,5- | | | | |
| | dimethyl-N-[2-(2-oxopiperazin-1-yl)- | | | | |
| | 4-pyridyl]-5- | | | | |
| | (trifluoromethyl)tetrahydrofuran-2- | | | | |
| | carboxamide | | | | |
| 516 | (2R,3S,4S,5R)-3-[2- | A | 578.479 | 579 | 3.27 |
| | (difluoromethoxy)-3,4-difluoro- | | | | |
| | phenyl]-4,5-dimethyl-N-[2-(4-methyl- | | | | |
| | 2-oxo-piperazin-1-yl)-4-pyridyl]-5- | | | | |
| | (trifluoromethyl)tetrahydrofuran-2- | | | | |
| | carboxamide | | | | |
| 517 | (2R,3S,4S,5R)-3-[2- | A | 592.506 | 593 | 3.36 |
| | (difluoromethoxy)-3,4-difluoro- | | | | |
| | phenyl]-N-[2-[(2R)-2,4-dimethyl-6- | | | | |
| | oxo-piperazin-1-yl]-4-pyridyl]-4,5- | | | | |
| | dimethyl-5- | | | | |
| | (trifluoromethyl)tetrahydrofuran-2- | | | | |
| | carboxamide | | | | |
| 518 | (2R,3S,4S,5R)-3-[3,4-difluoro-2- | A | 545.517 | 546 | 3.23 |
| | (trideuteriomethoxy)phenyl]-4,5- | | | | |
| | dimethyl-N-[2-(4-methyl-2-oxo- | | | | |
| | piperazin-1-yl)-4-pyridyl]-5- | | | | |
| | (trifluoromethyl)tetrahydrofuran-2- | | | | |
| | carboxamide | | | | |
| 519 | (2R,3S,4S,5R)-3-[3,4-difluoro-2- | A | 559.543 | 560 | 3.32 |
| | (trideuteriomethoxy)phenyl]-N-[2- | | | | |
| | [(2R)-2,4-dimethyl-6-oxo-piperazin-1- | | | | |
| | yl]-4-pyridyl]-4,5-dimethyl-5- | | | | |
| | (trifluoromethyl)tetrahydrofuran-2- | | | | |
| | carboxamide | | | | |
| 520 | (2R,3S,4S,5R)-3-(3,4-difluoro-2- | A | 529.5 | 530 | 3.47 |
| | methoxy-phenyl)-N-[2-[(2R)-2- | | | | |
| | (hydroxymethyl)pyrrolidin-1-yl]-4- | | | | |
| | pyridyl]-4,5-dimethyl-5- | | | | |
| | (trifluoromethyl)tetrahydrofuran-2- | | | | |
| | carboxamide | | | | |
| 521 | (2R,3S,4S,5R)-3-(3,4-difluoro-2- | A | 503.462 | 504 | 3.29 |
| | methoxy-phenyl)-N-[2-[2- | | | | |
| | hydroxyethyl(methyl)amino]-4- | | | | |
| | pyridyl]-4,5-dimethyl-5- | | | | |
| | (trifluoromethyl)tetrahydrofuran-2- | | | | |
| | carboxamide | | | | |
| 522 | (2R,3S,4S,5R)-3-(3,4-difluoro-2- | F | 544.514 | 545.21 | 2.53 |
| | methoxy-phenyl)-N-[2-[[2- | | | | |
| | (dimethylamino)acetyl]-methyl- | | | | |
| | amino]-4-pyridyl]-4,5-dimethyl-5- | | | | |
| | (trifluoromethyl)tetrahydrofuran-2- | | | | |
| | carboxamide | | | | |
| 523 | (2R,3S,4S,5R)-N-(6-amino-3- | A | 445.383 | 446 | 3.15 |
| | pyridyl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 524 | (2R,3S,4S,5R)-3-(3,4-difluoro-2- | A | 459.41 | 460 | 3.33 |
| | methoxy-phenyl)-4,5-dimethyl-N-[6- | | | | |
| | (methylamino)-3-pyridyl]-5- | | | | |
| | (trifluoromethyl)tetrahydrofuran-2- | | | | |
| | carboxamide | | | | |
| 525 | (2R,3S,4S,5R)-N-(6-acetamido-3- | A | 487.42 | 488 | 3.25 |
| | pyridyl)-3-(3,4-difluoro-2-methoxy- | | | | |
| | phenyl)-4,5-dimethyl-5- | | | | |
| | (trifluoromethyl)tetrahydrofuran-2- | | | | |
| | carboxamide | | | | |
| 526 | (2R,3S,4S,5R)-3-(3,4-difluoro-2- | A | 523.473 | 524 | 3.1 |
| | methoxy-phenyl)-N-[6- | | | | |
| | (methanesulfonamido)-3-pyridyl]-4,5- | | | | |
| | dimethyl-5- | | | | |
| | (trifluoromethyl)tetrahydrofuran-2- | | | | |
| | carboxamide | | | | |
| 527 | 3-(3,4-difluoro-2-methoxy-phenyl)-N- | E | 521.501 | 522.6 | 3.21 |
| | [2-(N,S-dimethylsulfonimidoyl)-4- | | | | |
| | pyridyl]-4,5-dimethyl-5- | | | | |
| | (trifluoromethyl)tetrahydrofuran-2- | | | | |
| | carboxamide | | | | |
| 528 | 3-(3,4-difluoro-2-methoxy-phenyl)-N- | A | 521.501 | 522.1 | 3.19 |
| | [2-(N,S-dimethylsulfonimidoyl)-4- | | | | |
| | pyridyl]-4,5-dimethyl-5- | | | | |
| | (trifluoromethyl)tetrahydrofuran-2- | | | | |
| | carboxamide | | | | |
| 529 | 3-(3,4-difluoro-2-methoxy-phenyl)-N- | A | 521.501 | 522.1 | 3.18 |
| | [2-(N,S-dimethylsulfonimidoyl)-4- | | | | |
| | pyridyl]-4,5-dimethyl-5- | | | | |
| | (trifluoromethyl)tetrahydrofuran-2- | | | | |
| | carboxamide | | | | |
| 530 | 3-(3,4-difluoro-2-methoxy-phenyl)-N- | A | 521.501 | 522.1 | 3.18 |
| | [2-(N,S-dimethylsulfonimidoyl)-4- | | | | |
| | pyridyl]-4,5-dimethyl-5- | | | | |
| | (trifluoromethyl)tetrahydrofuran-2- | | | | |
| | carboxamide | | | | |
| 531 | 3-(3,4-difluoro-2-methoxy-phenyl)-N- | A | 474.421 | 476.5 | 3.23 |
| | [2-(1-hydroxyethyl)-4-pyridyl]-4,5- | | | | |
| | dimethyl-5- | | | | |
| | (trifluoromethyl)tetrahydrofuran-2- | | | | |
| | carboxamide | | | | |
| 532 | 3-(3,4-difluoro-2-methoxy-phenyl)-N- | A | 474.421 | 475.8 | 3.23 |
| | [2-(1-hydroxyethyl)-4-pyridyl]-4,5- | | | | |
| | dimethyl-5- | | | | |
| | (trifluoromethyl)tetrahydrofuran-2- | | | | |
| | carboxamide | | | | |
| 533 | 3-(3,4-difluoro-2-methoxy-phenyl)-N- | A | 474.421 | 475.3 | 3.23 |
| | [2-(1-hydroxyethyl)-4-pyridyl]-4,5- | | | | |
| | dimethyl-5- | | | | |
| | (trifluoromethyl)tetrahydrofuran-2- | | | | |
| | carboxamide | | | | |
| 534 | 3-(3,4-difluoro-2-methoxy-phenyl)-N- | A | 474.421 | 475.6 | 3.23 |
| | [2-(1-hydroxyethyl)-4-pyridyl]-4,5- | | | | |
| | dimethyl-5- | | | | |
| | (trifluoromethyl)tetrahydrofuran-2- | | | | |
| | carboxamide | | | | |
| 535 | (2R,3S,4S,5R)-3-(3,4-difluoro-2- | F | 556.525 | 557.24 | 2.44 |
| | methoxy-phenyl)-N-[5-[(2R)-2,4- | | | | |
| | dimethyl-6-oxo-piperazin-1-yl]-3- | | | | |
| | pyridyl]-4,5-dimethyl-5- | | | | |
| | (trifluoromethyl)tetrahydrofuran-2- | | | | |
| | carboxamide | | | | |
| 536 | (2R,3S,4S,5R)-3-[3,4-difluoro-2-(2- | A | 513.406 | 514.6 | 3.17 |
| | hydroxyethoxy)phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 537 | (2R,3S,4S,5R)-3-[3,4-difluoro-2-(2- | A | 490.42 | 491.7 | 2.8 |
| | hydroxyethoxy)phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 538 | (2R,3S,4S,5R)-3-[3,4-difluoro-2-(2- | A | 541.489 | 542.6 | 3.07 |
| | hydroxyethoxy)phenyl]-4,5-dimethyl- | | | | |
| | N-(3-methyl-1-methylsulfonyl- | | | | |
| | pyrazol-4-yl)-5- | | | | |
| | (trifluoromethyl)tetrahydrofuran-2- | | | | |
| | carboxamide | | | | |
| 539 | (2R,3S,4S,5R)-3-[3,4-difluoro-2-(2- | A | 461.383 | 462.7 | 2.83 |
| | hydroxyethoxy)phenyl]-4,5-dimethyl- | | | | |
| | N-pyrimidin-5-yl-5- | | | | |
| | (trifluoromethyl)tetrahydrofuran-2- | | | | |
| | carboxamide | | | | |
| 540 | (2R,3S,4S,5R)-3-[3,4-difluoro-2-(2- | A | 518.474 | 519.8 | 3 |
| | hydroxyethoxy)phenyl]-N-[2-(1- | | | | |
| | hydroxy-1-methyl-ethyl)-4-pyridyl]- | | | | |
| | 4,5-dimethyl-5- | | | | |
| | (trifluoromethyl)tetrahydrofuran-2- | | | | |
| | carboxamide | | | | |
| 541 | (2R,3S,4S,5R)-3-[3,4-difluoro-2-(2- | A | 490.42 | 491.2 | 2.75 |
| | hydroxyethoxy)phenyl]-4,5-dimethyl- | | | | |
| | N-(1-methyl-2-oxo-4-pyridyl)-5- | | | | |
| | (trifluoromethyl)tetrahydrofuran-2- | | | | |
| | carboxamide | | | | |
| 542 | (2R,3S,4S,5R)-3-[3,4-difluoro-2-(2- | A | 546.487 | 547.3 | 3.05 |
| | hydroxyethoxy)phenyl]-4,5-dimethyl- | | | | |
| | N-(2-morpholinopyrimidin-5-yl)-5- | | | | |
| | (trifluoromethyl)tetrahydrofuran-2- | | | | |
| | carboxamide | | | | |
| 543 | (2R,3S,4S,5R)-3-[3,4-difluoro-2-(2- | A | 489.436 | 490.3 | 2.84 |
| | hydroxyethoxy)phenyl]-N-(2,4- | | | | |
| | dimethylpyrimidin-5-yl)-4,5- | | | | |
| | dimethyl-5- | | | | |
| | (trifluoromethyl)tetrahydrofuran-2- | | | | |
| | carboxamide | | | | |
| 544 | (2R,3S,4S,5R)-3-[3,4-difluoro-2-(2- | A | 490.42 | 491.2 | 3.17 |
| | hydroxyethoxy)phenyl]-N-(2- | | | | |
| | methoxy-4-pyridyl)-4,5-dimethyl-5- | | | | |
| | (trifluoromethyl)tetrahydrofuran-2- | | | | |
| | carboxamide | | | | |
| 545 | (2R,3S,4S,5R)-3-[3,4-difluoro-2-(2- | A | 464.383 | 465.59 | 3.03 |
| | hydroxyethoxy)phenyl]-4,5-dimethyl- | | | | |
| | N-(4-methylisoxazol-3-yl)-5- | | | | |
| | (trifluoromethyl)tetrahydrofuran-2- | | | | |
| | carboxamide | | | | |
| 546 | (2R,3S,4S,5R)-N-(5-cyclopropyl-1- | A | 503.462 | 504.6 | 3.19 |
| | methyl-pyrazol-3-yl)-3-[3,4-difluoro- | | | | |
| | 2-(2-hydroxyethoxy)phenyl]-4,5- | | | | |
| | dimethyl-5- | | | | |
| | (trifluoromethyl)tetrahydrofuran-2- | | | | |
| | carboxamide | | | | |
| 547 | (2R,3S,4S,5R)-3-[3,4-difluoro-2-(2- | A | 463.398 | 464.24 | 2.86 |
| | hydroxyethoxy)phenyl]-4,5-dimethyl- | | | | |
| | N-(2-methylpyrazol-3-yl)-5- | | | | |
| | (trifluoromethyl)tetrahydrofuran-2- | | | | |
| | carboxamide | | | | |
| 548 | (2R,3S,4S,5R)-3-[3,4-difluoro-2-(2- | A | 500.419 | 501.2 | 2.75 |
| | hydroxyethoxy)phenyl]-4,5-dimethyl- | | | | |
| | N-([1,2,4]triazolo[4,3-a]pyridin-6-yl)- | | | | |
| | 5-(trifluoromethyl)tetrahydrofuran-2- | | | | |
| | carboxamide | | | | |
| 549 | (2R,3S,4S,5R)-N-[1- | A | 488.408 | 489.2 | 2.92 |
| | (cyanomethyl)pyrazol-4-yl]-3-[3,4- | | | | |
| | difluoro-2-(2-hydroxyethoxy)phenyl]- | | | | |
| | 4,5-dimethyl-5- | | | | |
| | (trifluoromethyl)tetrahydrofuran-2- | | | | |
| | carboxamide | | | | |
| 550 | (2R,3S,4S,5R)-3-[3,4-difluoro-2-(2- | A | 491.408 | 492.2 | 2.95 |
| | hydroxyethoxy)phenyl]-N-(2- | | | | |
| | methoxypyrimidin-5-yl)-4,5-dimethyl- | | | | |
| | 5-(trifluoromethyl)tetrahydrofuran-2- | | | | |
| | carboxamide | | | | |
| 551 | (2R,3S,4S,5R)-3-[3,4-difluoro-2-(2- | A | 544.511 | 545.22 | 2.99 |
| | hydroxyethoxy)phenyl]-4,5-dimethyl- | | | | |
| | N-(2-methyl-5-tetrahydrofuran-3-yl-3- | | | | |
| | pyridyl)-5- | | | | |
| | (trifluoromethyl)tetrahydrofuran-2- | | | | |
| | carboxamide | | | | |
| 552 | (2R,3S,4S,5R)-3-[3,4-difluoro-2-(2- | A | 507.451 | 508.65 | 2.97 |
| | hydroxyethoxy)phenyl]-N-[1-(2- | | | | |
| | methoxyethyl)pyrazol-3-yl]-4,5- | | | | |
| | dimethyl-5- | | | | |
| | (trifluoromethyl)tetrahydrofuran-2- | | | | |
| | carboxamide | | | | |
| 553 | (2R,3S,4S,5R)-3-[3,4-difluoro-2-(2- | A | 494.412 | 495.25 | 2.71 |
| | hydroxyethoxy)phenyl]-N-[1- | | | | |
| | (methoxymethyl)-1,2,4-triazol-3-yl]- | | | | |
| | 4,5-dimethyl-5- | | | | |
| | (trifluoromethyl)tetrahydrofuran-2- | | | | |
| | carboxamide | | | | |
| 554 | (2R,3S,4S,5R)-3-[3,4-difluoro-2-(2- | A | 522.441 | 523.56 | 2.91 |
| | hydroxyethoxy)phenyl]-N-[1-(3- | | | | |
| | fluorocyclobutyl)-1,2,4-triazol-3-yl]- | | | | |
| | 4,5-dimethyl-5- | | | | |
| | (trifluoromethyl)tetrahydrofuran-2- | | | | |
| | carboxamide | | | | |
| 555 | (2R,3S,4S,5R)-3-[3,4-difluoro-2-(2- | A | 466.422 | 467.14 | 2.94 |
| | hydroxyethoxy)phenyl]-4,5-dimethyl- | | | | |
| | N-thiazol-5-yl-5- | | | | |
| | (trifluoromethyl)tetrahydrofuran-2- | | | | |
| | carboxamide | | | | |
| 556 | (2R,3S,4S,5R)-3-[3,4-difluoro-2-(2- | A | 450.357 | 451.48 | 3.01 |
| | hydroxyethoxy)phenyl]-N-isoxazol-3- | | | | |
| | yl-4,5-dimethyl-5- | | | | |
| | (trifluoromethyl)tetrahydrofuran-2- | | | | |
| | carboxamide | | | | |
| 557 | (2R,3S,4S,5R)-3-[3,4-difluoro-2-(2- | A | 477.425 | 478.24 | 2.86 |
| | hydroxyethoxy)phenyl]-N-(1,3- | | | | |
| | dimethylpyrazol-4-yl)-4,5-dimethyl-5- | | | | |
| | (trifluoromethyl)tetrahydrofuran-2- | | | | |
| | carboxamide | | | | |
| 558 | (2R,3S,4S,5R)-3-[3,4-difluoro-2-(2- | A | 464.383 | 465.24 | 3.04 |
| | hydroxyethoxy)phenyl]-4,5-dimethyl- | | | | |
| | N-(3-methylisoxazol-4-yl)-5- | | | | |
| | (trifluoromethyl)tetrahydrofuran-2- | | | | |
| | carboxamide | | | | |
| 559 | (2R,3S,4S,5R)-3-[3,4-difluoro-2-(2- | A | 464.383 | 465.19 | 3.02 |
| | hydroxyethoxy)phenyl]-4,5-dimethyl- | | | | |
| | N-(5-methylisoxazol-4-yl)-5- | | | | |
| | (trifluoromethyl)tetrahydrofuran-2- | | | | |
| | carboxamide | | | | |
| 560 | (2R,3S,4S,5R)-3-[3,4-difluoro-2-(2- | A | 474.421 | 475.29 | 3.01 |
| | hydroxyethoxy)phenyl]-4,5-dimethyl- | | | | |
| | N-(2-methyl-4-pyridyl)-5- | | | | |
| | (trifluoromethyl)tetrahydrofuran-2- | | | | |
| | carboxamide | | | | |
| 561 | (2R,3S,4S,5R)-3-[3,4-difluoro-2-(2- | A | 520.45 | 521.26 | 2.7 |
| | hydroxyethoxy)phenyl]-4,5-dimethyl- | | | | |
| | N-(1-tetrahydrofuran-3-yl-1,2,4- | | | | |
| | triazol-3-yl)-5- | | | | |
| | (trifluoromethyl)tetrahydrofuran-2- | | | | |
| | carboxamide | | | | |
| 562 | (2R,3S,4S,5R)-3-[3,4-difluoro-2-(2- | A | 519.462 | 520.26 | 3.04 |
| | hydroxyethoxy)phenyl]-N-[4- | | | | |
| | (methoxymethyl)-6-methyl-pyrimidin- | | | | |
| | 2-yl]-4,5-dimethyl-5- | | | | |
| | (trifluoromethyl)tetrahydrofuran-2- | | | | |
| | carboxamide | | | | |
| 563 | (2R,3S,4S,5R)-N-(1-tert- | A | 557.513 | 558.27 | 3.62 |
| | butylpyrazolo[3,4-d]pyrimidin-4-yl)- | | | | |
| | 3-[3,4-difluoro-2-(2- | | | | |
| | hydroxyethoxy)phenyl]-4,5-dimethyl- | | | | |
| | 5-(trifluoromethyl)tetrahydrofuran-2- | | | | |
| | carboxamide | | | | |
| 564 | (2R,3S,4S,5R)-3-[3,4-difluoro-2-(2- | A | 504.447 | 505.25 | 3 |
| | hydroxyethoxy)phenyl]-N-[2- | | | | |
| | (methoxymethyl)-4-pyridyl]-4,5- | | | | |
| | dimethyl-5- | | | | |
| | (trifluoromethyl)tetrahydrofuran-2- | | | | |
| | carboxamide | | | | |
| 565 | (2R,3S,4S,5R)-3-[3,4-difluoro-2-(2- | A | 515.433 | 516.31 | 2.91 |
| | hydroxyethoxy)phenyl]-4,5-dimethyl- | | | | |
| | N-(1-methyltriazolo[4,5-b]pyridin-6- | | | | |
| | yl)-5- | | | | |
| | (trifluoromethyl)tetrahydrofuran-2- | | | | |
| | carboxamide | | | | |
| 566 | (2R,3S,4S,5R)-3-[3,4-difluoro-2-(2- | A | 510.402 | 510.9 | 3.2 |
| | hydroxyethoxy)phenyl]-N-[2- | | | | |
| | (difluoromethyl)-4-pyridyl]-4,5- | | | | |
| | dimethyl-5- | | | | |
| | (trifluoromethyl)tetrahydrofuran-2- | | | | |
| | carboxamide | | | | |
| 567 | (2R,3S,4S,5R)-3-[3,4-difluoro-2-(2- | A | 534.473 | 535.26 | 3.14 |
| | hydroxyethoxy)phenyl]-N-[2-(2- | | | | |
| | methoxyethoxy)-4-pyridyl]-4,5- | | | | |
| | dimethyl-5- | | | | |
| | (trifluoromethyl)tetrahydrofuran-2- | | | | |
| | carboxamide | | | | |
| 568 | (2R,3S,4S,5R)-3-[3,4-difluoro-2-(2- | A | 491.408 | 492.3 | 2.84 |
| | hydroxyethoxy)phenyl]-4,5-dimethyl- | | | | |
| | N-(1-methyl-6-oxo-pyridazin-4-yl)-5- | | | | |
| | (trifluoromethyl)tetrahydrofuran-2- | | | | |
| | carboxamide | | | | |
| 569 | (2R,3S,4S,5R)-3-[3,4-difluoro-2-(2- | A | 475.409 | 476.64 | 2.83 |
| | hydroxyethoxy)phenyl]-4,5-dimethyl- | | | | |
| | N-(2-methylpyrimidin-5-yl)-5- | | | | |
| | (trifluoromethyl)tetrahydrofuran-2- | | | | |
| | carboxamide | | | | |
| 570 | (2R,3S,4S,5R)-3-[3,4-difluoro-2-(2- | A | 508.411 | 509.3 | 3.39 |
| | hydroxyethoxy)phenyl]-N-(5-fluoro- | | | | |
| | 2-methoxy-4-pyridyl)-4,5-dimethyl-5- | | | | |
| | (trifluoromethyl)tetrahydrofuran-2- | | | | |
| | carboxamide | | | | |
| 571 | (2R,3S,4S,5R)-3-[3,4-difluoro-2-(2- | A | 510.402 | 511.2 | 3.21 |
| | hydroxyethoxy)phenyl]-N-[6- | | | | |
| | (difluoromethyl)-3-pyridyl]-4,5- | | | | |
| | dimethyl-5- | | | | |
| | (trifluoromethyl)tetrahydrofuran-2- | | | | |
| | carboxamide | | | | |
| 572 | (2R,3S,4S,5R)-3-[3,4-difluoro-2-(2- | A | 466.422 | 466.8 | 3.24 |
| | hydroxyethoxy)phenyl]-N-isothiazol- | | | | |
| | 4-yl-4,5-dimethyl-5- | | | | |
| | (trifluoromethyl)tetrahydrofuran-2- | | | | |
| | carboxamide | | | | |
| 573 | 3-[3,4-difluoro-2-(2- | A | 464.386 | 465.2 | 2.94 |
| | hydroxyethoxy)phenyl]-4,5-dimethyl- | | | | |
| | N-(3-methyltriazol-4-yl)-5- | | | | |
| | (trifluoromethyl)tetrahydrofuran-2- | | | | |
| | carboxamide | | | | |
| 574 | (2R,3S,4S,5R)-3-[3,4-difluoro-2-(2- | A | 514.445 | 515.2 | 3.01 |
| | hydroxyethoxy)phenyl]-4,5-dimethyl- | | | | |
| | N-(3-methyltriazolo[1,5-a]pyridin-5- | | | | |
| | yl)-5- | | | | |
| | (trifluoromethyl)tetrahydrofuran-2- | | | | |
| | carboxamide | | | | |
| 575 | (2R,3S,4S,5R)-3-[3,4-difluoro-2-(2- | A | 460.394 | 461.7 | 2.93 |
| | hydroxyethoxy)phenyl]-4,5-dimethyl- | | | | |
| | N-(3-pyridyl)-5- | | | | |
| | (trifluoromethyl)tetrahydrofuran-2- | | | | |
| | carboxamide | | | | |
| 576 | (2R,3S,4S,5R)-3-[3,4-difluoro-2-(2- | A | 461.383 | 462 | 2.81 |
| | hydroxyethoxy)phenyl]-4,5-dimethyl- | | | | |
| | N-pyridazin-4-yl-5- | | | | |
| | (trifluoromethyl)tetrahydrofuran-2- | | | | |
| | carboxamide | | | | |
| 577 | (2R,3S,4S,5R)-3-[3,4-difluoro-2-(2- | A | 514.445 | 514.9 | 3.28 |
| | methoxyethoxy)phenyl]-4,5-dimethyl- | | | | |
| | N-([1,2,4]triazolo[4,3-a]pyridin-6-yl)- | | | | |
| | 5-(trifluoromethyl)tetrahydrofuran-2- | | | | |
| | carboxamide | | | | |
| 578 | (2R,3S,4S,5R)-3-[3,4-difluoro-2- | A | 486.432 | 487.2 | 3.11 |
| | (oxetan-3-ylmethoxy)phenyl]-4,5- | | | | |
| | dimethyl-N-(3-pyridyl)-5- | | | | |
| | (trifluoromethyl)tetrahydrofuran-2- | | | | |
| | carboxamide | | | | |
| 579 | (2R,3S,4S,5R)-N-[1-(difluoromethyl)- | A | 539.443 | 540.2 | 3.34 |
| | 3-methyl-pyrazol-4-yl]-3-[3,4- | | | | |
| | difluoro-2-(oxetan-3- | | | | |
| | ylmethoxy)phenyl]-4,5-dimethyl-5- | | | | |
| | (trifluoromethyl)tetrahydrofuran-2- | | | | |
| | carboxamide | | | | |
| 580 | 5-[[(2R,3S,4S,5R)-3-[3,4-difluoro-2- | C | 529.456 | 530.2 | 3.04 |
| | (oxetan-3-ylmethoxy)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 581 | (2R,3S,4S,5R)-3-(3,4-difluoro-2- | LCMS | 544.511 | 545.2 | 3.15 |
| | (oxetan-3-ylmethoxy)phenyl)-N-(6- | | | | |
| | (2-hydroxypropan-2-yl)pyridin-3-yl)- | | | | |
| | 4,5-dimethyl-5- | | | | |
| | (trifluoromethyl)tetrahydrofuran-2- | | | | |
| | carboxamide (TFA salt) | | | | |
| 582 | (2R,3S,4S,5R)-3-[3,4-difluoro-2-(2- | A | 535.504 | 534.3 | 3.24 |
| | methoxyethoxy)phenyl]-N-[1-(2- | | | | |
| | hydroxy-1,1-dimethyl-ethyl)pyrazol- | | | | |
| | 4-yl]-4,5-dimethyl-5- | | | | |
| | (trifluoromethyl)tetrahydrofuran-2- | | | | |
| | carboxamide | | | | |
| 583 | (2R,3S,4S,5R)-3-[3,4-difluoro-2-(2- | A | 535.504 | 536.3 | 3.21 |
| | methoxyethoxy)phenyl]-N-[1-(2- | | | | |
| | hydroxy-2-methyl-propyl)pyrazol-4- | | | | |
| | yl]-4,5-dimethyl-5- | | | | |
| | (trifluoromethyl)tetrahydrofuran-2- | | | | |
| | carboxamide | | | | |
| 584 | (2R,3S,4S,5R)-3-[3,4-difluoro-2-(2- | A | 504.447 | 504.9 | 3.07 |
| | methoxyethoxy)phenyl]-4,5-dimethyl- | | | | |
| | N-(1-methyl-6-oxo-3-pyridyl)-5- | | | | |
| | (trifluoromethyl)tetrahydrofuran-2- | | | | |
| | carboxamide | | | | |
| 585 | (2R,3S,4S,5R)-3-[3,4-difluoro-2-(2- | A | 586.551 | 587.1 | 3.21 |
| | methoxyethoxy)phenyl]-4,5-dimethyl- | | | | |
| | N-[2-(4-methyl-2-oxo-piperazin-1-yl)- | | | | |
| | 4-pyridyl]-5- | | | | |
| | (trifluoromethyl)tetrahydrofuran-2- | | | | |
| | carboxamide | | | | |
| 586 | (2R,3S,4S,5R)-3-[3,4-difluoro-2-(2- | A | 533.488 | 534.2 | 3.28 |
| | morpholinoethoxy)phenyl]-4,5- | | | | |
| | dimethyl-N-(3-methylisoxazol-4-yl)- | | | | |
| | 5-(trifluoromethyl)tetrahydrofuran-2- | | | | |
| | carboxamide | | | | |
| 587 | (2R,3S,4S,5R)-3-[3,4-difluoro-2-(2- | A | 529.5 | 530.2 | 3.18 |
| | morpholinoethoxy)phenyl]-4,5- | | | | |
| | dimethyl-N-(3-pyridyl)-5- | | | | |
| | (trifluoromethyl)tetrahydrofuran-2- | | | | |
| | carboxamide | | | | |
| 588 | (2R,3S,4S,5R)-3-[3,4-difluoro-2-(2- | A | 559.526 | 560.3 | 2.99 |
| | morpholinoethoxy)phenyl]-4,5- | | | | |
| | dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 589 | (2R,3S,4S,5R)-3-[3,4-difluoro-2-(2- | A | 559.526 | 560 | 3.24 |
| | morpholinoethoxy)phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 590 | (2R,3S,4S,5R)-3-[3,4-difluoro-2-(2- | A | 569.524 | 570.3 | 3 |
| | morpholinoethoxy)phenyl]-4,5- | | | | |
| | dimethyl-N-([1,2,4]triazolo[4,3- | | | | |
| | alpyridin-6-yl)-5- | | | | |
| | (trifluoromethyl)tetrahydrofuran-2- | | | | |
| | carboxamide | | | | |
| 591 | (2R,3S,4S,5R)-N-[1-(difluoromethyl)- | A | 582.511 | 583.3 | 3.41 |
| | 3-methyl-pyrazol-4-yl]-3-[3,4- | | | | |
| | difluoro-2-(2- | | | | |
| | morpholinoethoxy)phenyl]-4,5- | | | | |
| | dimethyl-5- | | | | |
| | (trifluoromethyl)tetrahydrofuran-2- | | | | |
| | carboxamide | | | | |
| 592 | 5-[[(2R,3S,4S,5R)-3-[3,4-difluoro-2- | A | 575.528 | 576.3 | 2.92 |
| | (2-morpholinoethoxy)phenyl]-4,5- | | | | |
| | dimethyl-5- | | | | |
| | (trifluoromethyl)tetrahydrofuran-2- | | | | |
| | carbonyl]amino]-1-methyl-pyrazole- | | | | |
| | 3-carboxamide | | | | |
| 593 | 4-[[(2R,3S,4S,5R)-3-[3,4-difluoro-2- | A | 575.528 | 576.3 | 3.12 |
| | (2-morpholinoethoxy)phenyl]-4,5- | | | | |
| | dimethyl-5- | | | | |
| | (trifluoromethyl)tetrahydrofuran-2- | | | | |
| | carbonyl]amino]-3-methyl-pyrazole- | | | | |
| | 1-carboxamide | | | | |
| 594 | (2R,3S,4S,5R)-3-[3,4-difluoro-2-(2- | A | 588.567 | 589.2 | 3.11 |
| | morpholinoethoxy)phenyl]-4,5- | | | | |
| | dimethyl-N-[3-methyl-1-(oxetan-3- | | | | |
| | yl)pyrazol-4-yl]-5- | | | | |
| | (trifluoromethyl)tetrahydrofuran-2- | | | | |
| | carboxamide | | | | |
| 595 | (2R,3S,4S,5R)-3-[3,4-difluoro-2-(2- | A | 603.578 | 604.3 | 3.1 |
| | morpholinoethoxy)phenyl]-N-[2-(1- | | | | |
| | hydroxy-2-methoxy-ethyl)-4-pyridyl]- | | | | |
| | 4,5-dimethyl-5- | | | | |
| | (trifluoromethyl)tetrahydrofuran-2- | | | | |
| | carboxamide | | | | |
| 596 | (2R,3S,4S,5R)-3-[3,4-difluoro-2-(2- | A | 617.605 | 618.2 | 3.11 |
| | morpholinoethoxy)phenyl]-N-[2- | | | | |
| | [(2S)-2-hydroxy-3-methoxy-propyl]- | | | | |
| | 4-pyridyl]-4,5-dimethyl-5- | | | | |
| | (trifluoromethyl)tetrahydrofuran-2- | | | | |
| | carboxamide | | | | |
| 597 | 5-[[(2R,3S,4S,5R)-3-[2-[2- | A | 601.565 | 602.1 | 3 |
| | [(3aS,6aR)-1,3,3a,4,6,6a- | | | | |
| | hexahydrofuro[3,4-c]pyrrol-5- | | | | |
| | yl]ethoxy]-3,4-difluoro-phenyl]-4,5- | | | | |
| | dimethyl-5- | | | | |
| | (trifluoromethyl)tetrahydrofuran-2- | | | | |
| | carbonyl]amino]-1-methyl-pyrazole- | | | | |
| | 3-carboxamide | | | | |
| 598 | (2R,3S,4S,5R)-N-[1-(difluoromethyl)- | F | 594.522 | 595.2 | 2.49 |
| | 3-methyl-pyrazol-4-yl]-3-[3,4- | | | | |
| | difluoro-2-[2-(2-oxa-6- | | | | |
| | azaspiro[3.3]heptan-6- | | | | |
| | yl)ethoxy]phenyl]-4,5-dimethyl-5- | | | | |
| | (trifluoromethyl)tetrahydrofuran-2- | | | | |
| | carboxamide | | | | |
| 599 | (2R,3S,4S,5R)-N-[1-(difluoromethyl)- | A | 549.441 | 550.1 | 3.18 |
| | 3-methyl-pyrazol-4-yl]-3-[3,4- | | | | |
| | difluoro-2-(1H-pyrazol-4- | | | | |
| | ylmethoxy)phenyl]-4,5-dimethyl-5- | | | | |
| | (trifluoromethyl)tetrahydrofuran-2- | | | | |
| | carboxamide | | | | |
| 600 | (2R,3S,4S,5R)-3-[3,4-difluoro-2-(3- | A | 539.443 | 540.6 | 3.28 |
| | hydroxycyclobutoxy)phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 601 | (2R,3S,4S,5R)-3-[3,4-difluoro-2-[2- | F | 539.443 | 540.2 | 2.55 |
| | (hydroxymethyl)allyloxy]phenyl]-N- | | | | |
| | [1-(difluoromethyl)-3-methyl-pyrazol- | | | | |
| | 4-yl]-4,5-dimethyl-5- | | | | |
| | (trifluoromethyl)tetrahydrofuran-2- | | | | |
| | carboxamide | | | | |
| 602 | methyl 2-[6-[(2R,3S,4S,5R)-2-[[1- | A | 541.416 | 542 | 3.52 |
| | (difluoromethyl)-3-methyl-pyrazol-4- | | | | |
| | yl]carbamoyl]-4,5-dimethyl-5- | | | | |
| | (trifluoromethyl)tetrahydrofuran-3- | | | | |
| | yl]-2,3-difluoro-phenoxy]acetate | | | | |
| 603 | 2-[6-[(2R,3S,4S,5R)-2-[[1- | A | 527.389 | 528.3 | 2.26 |
| | (difluoromethyl)-3-methyl-pyrazol-4- | | | | |
| | yl]carbamoyl]-4,5-dimethyl-5- | | | | |
| | (trifluoromethyl)tetrahydrofuran-3- | | | | |
| | yl]-2,3-difluoro-phenoxy]acetic acid | | | | |
| 604 | (2R,3S,4S,5R)-3-[3,4-difluoro-2-[2- | A | 556.431 | 557.3 | 3.09 |
| | [hydroxy(methyl)amino]-2-oxo- | | | | |
| | ethoxy]phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 605 | (2R,3S,4S,5R)-3-[2-(2-amino-2-oxo- | A | 526.405 | 527.3 | 2.94 |
| | ethoxy)-3,4-difluoro-phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 606 | (2R,3S,4S,5R)-3-[3,4-difluoro-2-[2- | E | 542.404 | 543.4 | 3.01 |
| | (hydroxyamino)-2-oxo- | | | | |
| | ethoxy]phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 607 | (2R,3S,4S,5R)-3-[3,4-difluoro-2-[2- | A | 540.431 | 541.4 | 3.05 |
| | (methylamino)-2-oxo-ethoxy]phenyl]- | | | | |
| | N-[1-(difluoromethyl)-3-methyl- | | | | |
| | pyrazol-4-yl]-4,5-dimethyl-5- | | | | |
| | (trifluoromethyl)tetrahydrofuran-2- | | | | |
| | carboxamide | | | | |
| 608 | (2R,3S,4S,5R)-3-[3,4-difluoro-2-[2- | A | 591.543 | 591.9 | 3.19 |
| | (3-fluoroazetidin-1-yl)ethoxy]phenyl]- | | | | |
| | N-[2-(1-hydroxy-2-methoxy-ethyl)-4- | | | | |
| | pyridyl]-4,5-dimethyl-5- | | | | |
| | (trifluoromethyl)tetrahydrofuran-2- | | | | |
| | carboxamide | | | | |
| 609 | (2R,3S,4S,5R)-3-[3,4-difluoro-2-[2- | A | 591.543 | 591.9 | 3.19 |
| | (3-fluoroazetidin-1-yl)ethoxy]phenyl]- | | | | |
| | N-[2-(1-hydroxy-2-methoxy-ethyl)-4- | | | | |
| | pyridyl]-4,5-dimethyl-5- | | | | |
| | (trifluoromethyl)tetrahydrofuran-2- | | | | |
| | carboxamide | | | | |
| 610 | (2R,3S,4S,5R)-3-[3,4-difluoro-2-[2- | A | 615.589 | 615.9 | 3.12 |
| | (6-oxa-3-azabicyclo[3.1.1]heptan-3- | | | | |
| | yl)ethoxy]phenyl]-N-[2-(1-hydroxy-2- | | | | |
| | methoxy-ethyl)-4-pyridyl]-4,5- | | | | |
| | dimethyl-5- | | | | |
| | (trifluoromethyl)tetrahydrofuran-2- | | | | |
| | carboxamide | | | | |
| 611 | (2R,3S,4S,5R)-3-[3,4-difluoro-2-[2- | A | 615.589 | 615.9 | 3.12 |
| | (6-oxa-3-azabicyclo[3.1.1]heptan-3- | | | | |
| | yl)ethoxy phenyl]-N-[2-(1-hydroxy-2- | | | | |
| | methoxy-ethyl)-4-pyridyl]-4,5- | | | | |
| | dimethyl-5- | | | | |
| | (trifluoromethyl)tetrahydrofuran-2- | | | | |
| | carboxamide | | | | |
| 612 | 5-[[(2R,3S,4S,5R)-3-[3,4-difluoro-2- | A | 584.535 | 585 | 2.97 |
| | [2-(2-oxa-6-azaspiro[3.3]heptan-6- | | | | |
| | yl)ethoxyphenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 613 | 5-[[(2R,3S,4S,5R)-3-[3,4-difluoro-2- | A | 598.562 | 599 | 3.05 |
| | [2-(2-oxa-6-azaspiro[3.3]heptan-6- | | | | |
| | yl)ethoxyphenyl]-4,5-dimethyl-5- | | | | |
| | (trifluoromethyl)tetrahydrofuran-2- | | | | |
| | carbonyl]amino]-N-methyl-pyridine- | | | | |
| | 2-carboxamide | | | | |
| 614 | 5-[[(2R,3S,4S,5R)-3-[3,4-difluoro-2- | A | 531.472 | 532.7 | 3.14 |
| | [(2R)-3-hydroxy-2-methyl- | | | | |
| | propoxy]phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 615 | 5-[[(2R,3S,4S,5R)-3-[3,4-difluoro-2- | A | 503.419 | 504.3 | 2.9 |
| | (2-hydroxyethoxy)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 616 | 5-[[(2R,3S,4S,5R)-3-[3,4-difluoro-2- | A | 531.472 | 532.3 | 3.12 |
| | [(2S)-3-hydroxy-2-methyl- | | | | |
| | propoxy]phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 617 | (2R,3S,4S,5R)-3-[3,4-difluoro-2-[2- | F | 581.534 | 582.21 | 2.22 |
| | (2-oxa-6-azaspiro[3.3]heptan-6- | | | | |
| | yl)ethoxy]phenyl]-4,5-dimethyl-N- | | | | |
| | (triazolo[1,5-a]pyridin-6-yl)-5- | | | | |
| | (trifluoromethyl)tetrahydrofuran-2- | | | | |
| | carboxamide | | | | |
| 618 | (2R,3S,4S,5R)-3-[3,4-difluoro-2-[2- | A | 571.536 | 572.3 | 2.9 |
| | (2-oxa-6-azaspiro[3.3]heptan-6- | | | | |
| | yl)ethoxy]phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 619 | (2R,3S,4S,5R)-3-[3,4-difluoro-2-[2- | A | 545.499 | 545.9 | 3.11 |
| | (2-oxa-6-azaspiro[3.3]heptan-6- | | | | |
| | yl)ethoxyphenyl]-4,5-dimethyl-N-(3- | | | | |
| | methylisoxazol-4-yl)-5- | | | | |
| | (trifluoromethyl)tetrahydrofuran-2- | | | | |
| | carboxamide | | | | |
| 620 | (2R,3S,4S,5R)-3-[3,4-difluoro-2-[2- | A | 581.534 | 582.2 | 2.85 |
| | (2-oxa-6-azaspiro[3.3]heptan-6- | | | | |
| | yl)ethoxy]phenyl]-4,5-dimethyl-N- | | | | |
| | ([1,2,4]triazolo[4,3-a]pyridin-6-yl)-5- | | | | |
| | (trifluoromethyl)tetrahydrofuran-2- | | | | |
| | carboxamide | | | | |
| 621 | (2R,3S,4S,5R)-3-[3,4-difluoro-2- | F | 526.456 | 527.17 | 2.43 |
| | (oxetan-3-ylmethoxy)phenyl]-4,5- | | | | |
| | dimethyl-N-(triazolo[1,5-a]pyridin-6- | | | | |
| | yl)-5- | | | | |
| | (trifluoromethyl)tetrahydrofuran-2- | | | | |
| | carboxamide | | | | |
| 622 | (2R,3S,4S,5R)-3-[3,4-difluoro-2-[2- | F | 526.456 | 527.18 | 2.33 |
| | (hydroxymethyl)allyloxy]phenyl]-4,5- | | | | |
| | dimethyl-N-(triazolo[1,5-a]pyridin-6- | | | | |
| | yl)-5- | | | | |
| | (trifluoromethyl)tetrahydrofuran-2- | | | | |
| | carboxamide | | | | |
| 623 | (2R,3S,4S,5R)-3-[3,4-difluoro-2- | F | 559.526 | 560.27 | 2.52 |
| | [(2S)-2-(2-oxa-6-azaspiro[3.3]heptan- | | | | |
| | 6-yl)propoxy]phenyl]-4,5-dimethyl-N- | | | | |
| | (3-methylisoxazol-4-yl)-5- | | | | |
| | (trifluoromethyl)tetrahydrofuran-2- | | | | |
| | carboxamide | | | | |
| 624 | (2R,3S,4S,5R)-3-[3,4-difluoro-2- | F | 559.526 | 560.29 | 2.46 |
| | [(2R)-2-(2-oxa-6-azaspiro[3.3]heptan- | | | | |
| | 6-yl)propoxy]phenyl]-4,5-dimethyl-N- | | | | |
| | (3-methylisoxazol-4-yl)-5- | | | | |
| | (trifluoromethyl)tetrahydrofuran-2- | | | | |
| | carboxamide | | | | |
| 625 | (2R,3S,4S,5R)-3-[3,4-difluoro-2-[2- | A | 521.453 | 522.7 | 3.4 |
| | (3-fluoroazetidin-1-yl)ethoxy]phenyl]- | | | | |
| | 4,5-dimethyl-N-(3-methylisoxazol-4- | | | | |
| | yl)-5- | | | | |
| | (trifluoromethyl)tetrahydrofuran-2- | | | | |
| | carboxamide | | | | |
| 626 | (2R,3S,4S,5R)-3-[3,4-difluoro-2-[2- | A | 533.488 | 533.7 | 2.98 |
| | (3-hydroxy-3-methyl-azetidin-1- | | | | |
| | yl)ethoxyphenyl]-4,5-dimethyl-N-(3- | | | | |
| | methylisoxazol-4-yl)-5- | | | | |
| | (trifluoromethyl)tetrahydrofuran-2- | | | | |
| | carboxamide | | | | |
| 627 | (2R,3S,4S,5R)-3-[3,4-difluoro-2-[2- | A | 533.488 | 535 | 3.32 |
| | (3-methoxyazetidin-1- | | | | |
| | yl)ethoxy]phenyl]-4,5-dimethyl-N-(3- | | | | |
| | methylisoxazol-4-yl)-5- | | | | |
| | (trifluoromethyl)tetrahydrofuran-2- | | | | |
| | carboxamide | | | | |
| 628 | (2R,3S,4S,5R)-3-[2-[2-[3- | A | 569.469 | 570.1 | 3.53 |
| | (difluoromethoxy)azetidin-1- | | | | |
| | yl]ethoxy]-3,4-difluoro-phenyl]-4,5- | | | | |
| | dimethyl-N-(3-methylisoxazol-4-yl)- | | | | |
| | 5-(trifluoromethyl)tetrahydrofuran-2- | | | | |
| | carboxamide | | | | |
| 629 | (2R,3S,4S,5R)-3-[2-[2-[3- | A | 553.47 | 554.9 | 3.5 |
| | (difluoromethyl)azetidin-1-yl]ethoxy]- | | | | |
| | 3,4-difluoro-phenyl]-4,5-dimethyl-N- | | | | |
| | (3-methylisoxazol-4-yl)-5- | | | | |
| | (trifluoromethyl)tetrahydrofuran-2- | | | | |
| | carboxamide | | | | |
| 630 | (2R,3S,4S,5R)-3-[3,4-difluoro-2-[2- | A | 535.479 | 536.6 | 3.52 |
| | (3-fluoro-3-methyl-azetidin-1- | | | | |
| | yl)ethoxy\|phenyl]-4,5-dimethyl-N-(3- | | | | |
| | methylisoxazol-4-yl)-5- | | | | |
| | (trifluoromethyl)tetrahydrofuran-2- | | | | |
| | carboxamide | | | | |
| 631 | (2R,3S,4S,5R)-3-[3,4-difluoro-2-[2- | A | 519.462 | 519.9 | 2.98 |
| | (oxetan-3-ylamino)ethoxyphenyl]- | | | | |
| | 4,5-dimethyl-N-(3-methylisoxazol-4- | | | | |
| | yl)-5- | | | | |
| | (trifluoromethyl)tetrahydrofuran-2- | | | | |
| | carboxamide | | | | |
| 632 | (2R,3S,4S,5R)-3-[3,4-difluoro-2-[2- | A | 533.488 | 534.1 | 3.1 |
| | [[(3R)-tetrahydrofuran-3- | | | | |
| | yl]amino Jethoxy]phenyl]-4,5- | | | | |
| | dimethyl-N-(3-methylisoxazol-4-yl)- | | | | |
| | 5-(trifluoromethyl)tetrahydrofuran-2- | | | | |
| | carboxamide | | | | |
| 633 | (2R,3S,4S,5R)-3-[3,4-difluoro-2-[2- | A | 545.499 | 545.9 | 3.31 |
| | (6-oxa-3-azabicyclo[3.1.1]heptan-3- | | | | |
| | yl)ethoxy]phenyl]-4,5-dimethyl-N-(3- | | | | |
| | methylisoxazol-4-yl)-5- | | | | |
| | (trifluoromethyl)tetrahydrofuran-2- | | | | |
| | carboxamide | | | | |
| 634 | (2R,3S,4S,5R)-3-[2-[2-[(3aS,6aR)- | A | 559.526 | 559.7 | 3.36 |
| | 1,3,3a,4,6,6a-hexahydrofuro[3,4- | | | | |
| | c]pyrrol-5-yl]ethoxy]-3,4-difluoro- | | | | |
| | phenyl]-4,5-dimethyl-N-(3- | | | | |
| | methylisoxazol-4-yl)-5- | | | | |
| | (trifluoromethyl)tetrahydrofuran-2- | | | | |
| | carboxamide | | | | |
| 635 | (2R,3S,4S,5R)-3-[3,4-difluoro-2-[2- | A | 557.488 | 558.2 | 3.1 |
| | (3-fluoroazetidin-1-yl)ethoxy]phenyl]- | | | | |
| | 4,5-dimethyl-N-([1,2,4]triazolo[4,3- | | | | |
| | alpyridin-6-yl)-5- | | | | |
| | (trifluoromethyl)tetrahydrofuran-2- | | | | |
| | carboxamide | | | | |
| 636 | (2R,3S,4S,5R)-3-[3,4-difluoro-2-[2- | A | 541.51 | 542.7 | 3.08 |
| | (2-oxa-6-azaspiro[3.3]heptan-6- | | | | |
| | yl)ethoxyphenyl]-4,5-dimethyl-N-(3- | | | | |
| | pyridyl)-5- | | | | |
| | (trifluoromethyl)tetrahydrofuran-2- | | | | |
| | carboxamide | | | | |
| 637 | (2R,3S,4S,5R)-N-[2- | A | 612.588 | 613 | 3.03 |
| | [acetyl(methyl)amino]-4-pyridyl]-3- | | | | |
| | [3,4-difluoro-2-[2-(2-oxa-6- | | | | |
| | azaspiro[3.3]heptan-6- | | | | |
| | yl)ethoxy]phenyl]-4,5-dimethyl-5- | | | | |
| | (trifluoromethyl)tetrahydrofuran-2- | | | | |
| | carboxamide | | | | |
| 638 | (2R,3S,4S,5R)-3-(3,4-difluoro-2- | A | 446.368 | 446.9 | 3.24 |
| | hydroxy-phenyl)-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 639 | (2R,3S,4S,5R)-3-[3,4-difluoro-2-(2- | A [2], A | 504.447 | 505.2 | 3.15 |
| | methoxyethoxy)phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 640 | (2R,3S,4S,5R)-3-(2-(2- | A | 565.553 | 565.7 | 3.43 |
| | ((dimethyl(oxo)-16_{—} | | | | |
| | sulfaneylidene)amino)ethoxy)-3,4- | | | | |
| | difluorophenyl)-N-(2- | | | | |
| | (hydroxymethyl)pyridin-4-yl)-4,5- | | | | |
| | dimethyl-5- | | | | |
| | (trifluoromethyl)tetrahydrofuran-2- | | | | |
| | carboxamide | | | | |
| 641 | (2R,3S,4S,5R)-3-[2-[2- | A | 540.428 | 541.2 | 3.26 |
| | (difluoromethoxy)ethoxy]-3,4- | | | | |
| | difluoro-phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 642 | (2R,3S,4S,5R)-3-[3,4-difluoro-2-(3- | A | 518.474 | 519.3 | 3.23 |
| | methoxypropoxy)phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 643 | (2R,3S,4S,5R)-3-[2-(2,2- | A | 524.429 | 525.2 | 3.32 |
| | difluoropropoxy)-3,4-difluoro- | | | | |
| | phenyl]-N-[2-(hydroxymethyl)-4- | | | | |
| | pyridyl]-4,5-dimethyl-5- | | | | |
| | (trifluoromethyl)tetrahydrofuran-2- | | | | |
| | carboxamide | | | | |
| 644 | (2R,3S,4S,5R)-3-[2-[(3,3- | A | 550.466 | 551.2 | 3.48 |
| | difluorocyclobutyl)methoxy]-3,4- | | | | |
| | difluoro-phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 645 | (2R,3S,4S,5R)-3-[3,4-difluoro-2-[(3- | A | 534.448 | 535.2 | 3.06 |
| | fluorooxetan-3-yl)methoxy phenyl]- | | | | |
| | N-[2-(hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 646 | (2R,3S,4S,5R)-3-[3,4-difluoro-2- | A | 518.474 | 519.2 | 3.36 |
| | [(1R)-2-methoxy-1-methyl- | | | | |
| | ethoxy]phenyl]-N-[2- | | | | |
| | (hydroxymethyl)-4-pyridyl]-4,5- | | | | |
| | dimethyl-5- | | | | |
| | (trifluoromethyl)tetrahydrofuran-2- | | | | |
| | carboxamide | | | | |
| 647 | (2R,3S,4S,5R)-3-[3,4-difluoro-2-(2- | C | 534.473 | 535 | 0.87 |
| | methoxyethoxy)phenyl]-N-[2-(1,2- | | | | |
| | dihydroxyethyl)-4-pyridyl]-4,5- | | | | |
| | dimethyl-5- | | | | |
| | (trifluoromethyl)tetrahydrofuran-2- | | | | |
| | carboxamide | | | | |
| 648 | (2R,3S,4S,5R)-3-[3,4-difluoro-2-(2- | A | 518.474 | 519.3 | 3.16 |
| | methoxyethoxy)phenyl]-N-[2-(2- | | | | |
| | hydroxyethyl)-4-pyridyl]-4,5- | | | | |
| | dimethyl-5- | | | | |
| | (trifluoromethyl)tetrahydrofuran-2- | | | | |
| | carboxamide | | | | |
| 649 | 5-[[(2R,3S,4S,5R)-3-(3,4-difluoro-2- | A | 459.367 | 460.2 | 2.76 |
| | hydroxy-phenyl)-4,5-dimethyl-5- | | | | |
| | (trifluoromethyl)tetrahydrofuran-2- | | | | |
| | carbonyl]amino]pyridine-2- | | | | |
| | carboxamide | | | | |
| 650 | (2R,3S,4S,5R)-3-[2-[(1,4- | A | 524.483 | 525.9 | 3.37 |
| | dimethylimidazol-2-yl)methoxy]-3,4- | | | | |
| | difluoro-phenyl]-4,5-dimethyl-N-(3- | | | | |
| | pyridyl)-5- | | | | |
| | (trifluoromethyl)tetrahydrofuran-2- | | | | |
| | carboxamide | | | | |
| 651 | (2R,3S,4S,5R)-3-[2-[(1,5- | A | 524.483 | 525.8 | 3.77 |
| | dimethylimidazol-2-yl)methoxy]-3,4- | | | | |
| | difluoro-phenyl]-4,5-dimethyl-N-(3- | | | | |
| | pyridyl)-5- | | | | |
| | (trifluoromethyl)tetrahydrofuran-2- | | | | |
| | carboxamide | | | | |
| 652 | (2R,3S,4S,5R)-3-[3,4-difluoro-2-(2- | A | 600.577 | 601 | 3.33 |
| | methoxyethoxy)phenyl]-N-[2-[(2R)- | | | | |
| | 2,4-dimethyl-6-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 653 | (2R,3S,4S,5R)-3-[3,4-difluoro-2-(2- | F | 586.551 | 587.2 | 2.22 |
| | hydroxyethoxy)phenyl]-N-[2-[(2R)- | | | | |
| | 2,4-dimethyl-6-oxo-piperazin-1-yl]-4- | | | | |
| | pyridyl]-4,5-dimethyl-5- | | | | |
| | (trifluoromethyl)tetrahydrofuran-2- | | | | |
| | carboxamide | | | | |
| 654 | (2R,3S,4S,5R)-3-[3,4-difluoro-2-(3- | A | 486.432 | 487.6 | 3.06 |
| | hydroxycyclobutoxy)phenyl]-4,5- | | | | |
| | dimethyl-N-(3-pyridyl)-5- | | | | |
| | (trifluoromethyl)tetrahydrofuran-2- | | | | |
| | carboxamide | | | | |
| 655 | 5-[[(2R,3S,4S,5R)-3-[3,4-difluoro-2- | A | 529.456 | 530.6 | 3 |
| | (3-hydroxycyclobutoxy)phenyl]-4,5- | | | | |
| | dimethyl-5- | | | | |
| | (trifluoromethyl)tetrahydrofuran-2- | | | | |
| | carbonyl]amino]pyridine-2- | | | | |
| | carboxamide | | | | |
| 656 | (2R,3S,4S,5R)-3-[3,4-difluoro-2-(3- | A | 490.42 | 491.6 | 3.15 |
| | hydroxycyclobutoxy)phenyl]-4,5- | | | | |
| | dimethyl-N-(5-methylisoxazol-4-yl)- | | | | |
| | 5-(trifluoromethyl)tetrahydrofuran-2- | | | | |
| | carboxamide | | | | |
| 657 | (2R,3S,4S,5R)-3-[3,4-difluoro-2-(3- | A | 534.448 | 535.6 | 3.34 |
| | hydroxycyclobutoxy)phenyl]-N-[5- | | | | |
| | fluoro-2-(hydroxymethyl)-4-pyridyl]- | | | | |
| | 4,5-dimethyl-5- | | | | |
| | (trifluoromethyl)tetrahydrofuran-2- | | | | |
| | carboxamide | | | | |
| 658 | (2R,3S,4S,5R)-3-[3,4-difluoro-2-(3- | A | 487.42 | 488.5 | 2.96 |
| | hydroxycyclobutoxy)phenyl]-4,5- | | | | |
| | dimethyl-N-pyridazin-4-yl-5- | | | | |
| | (trifluoromethyl)tetrahydrofuran-2- | | | | |
| | carboxamide | | | | |
| 659 | (2R,3S,4S,5R)-N-(5-cyano-3-pyridyl)- | A | 511.441 | 512.6 | 3.23 |
| | 3-[3,4-difluoro-2-(3- | | | | |
| | hydroxycyclobutoxy)phenyl]-4,5- | | | | |
| | dimethyl-5- | | | | |
| | (trifluoromethyl)tetrahydrofuran-2- | | | | |
| | carboxamide | | | | |
| 660 | (2R,3S,4S,5R)-3-[3,4-difluoro-2-(3- | A | 504.422 | 505.5 | 3.25 |
| | hydroxycyclobutoxy)phenyl]-N-(5- | | | | |
| | fluoro-3-pyridyl)-4,5-dimethyl-5- | | | | |
| | (trifluoromethyl)tetrahydrofuran-2- | | | | |
| | carboxamide | | | | |
| 661 | (2R,3S,4S,5R)-3-[3,4-difluoro-2-(3- | A | 500.458 | 501.6 | 3.14 |
| | hydroxycyclobutoxy)phenyl]-4,5- | | | | |
| | dimethyl-N-(6-methyl-3-pyridyl)-5- | | | | |
| | (trifluoromethyl)tetrahydrofuran-2- | | | | |
| | carboxamide | | | | |
| 662 | (2R,3S,4S,5R)-3-[3,4-difluoro-2-(3- | A [2], A | 526.456 | 527.55 | 2.875 |
| | hydroxycyclobutoxy)phenyl]-4,5- | | | | |
| | dimethyl-N-([1,2,4]triazolo[4,3- | | | | |
| | alpyridin-7-yl)-5- | | | | |
| | (trifluoromethyl)tetrahydrofuran-2- | | | | |
| | carboxamide | | | | |
| 663 | (2R,3S,4S,5R)-3-[3,4-difluoro-2-(3- | A | 516.458 | 517.6 | 2.9 |
| | hydroxycyclobutoxy)phenyl]-4,5- | | | | |
| | dimethyl-N-(1-methyl-2-oxo-4- | | | | |
| | pyridyl)-5- | | | | |
| | (trifluoromethyl)tetrahydrofuran-2- | | | | |
| | carboxamide | | | | |
| 664 | (2R,3S,4S,5R)-3-[3,4-difluoro-2-(3- | A | 530.484 | 531.6 | 3.16 |
| | hydroxycyclobutoxy)phenyl]-N-[2- | | | | |
| | (methoxymethyl)-4-pyridyl]-4,5- | | | | |
| | dimethyl-5- | | | | |
| | (trifluoromethyl)tetrahydrofuran-2- | | | | |
| | carboxamide | | | | |
| 665 | (2R,3S,4S,5R)-3-[3,4-difluoro-2-(3- | A | 500.458 | 501.6 | 3.18 |
| | hydroxycyclobutoxy)phenyl]-4,5- | | | | |
| | dimethyl-N-(5-methyl-3-pyridyl)-5- | | | | |
| | (trifluoromethyl)tetrahydrofuran-2- | | | | |
| | carboxamide | | | | |
| 666 | (2R,3S,4S,5R)-3-[3,4-difluoro-2-(3- | A | 553.499 | | 3.19 |
| | hydroxycyclobutoxy)phenyl]-4,5- | | | | |
| | dimethyl-N-(1-methylsulfonylpyrazol- | | | | |
| | 4-yl)-5- | | | | |
| | (trifluoromethyl)tetrahydrofuran-2- | | | | |
| | carboxamide | | | | |
| 667 | (2R,3S,4S,5R)-3-[3,4-difluoro-2-(3- | A | 564.522 | 565.6 | 3.11 |
| | hydroxycyclobutoxy)phenyl]-4,5- | | | | |
| | dimethyl-N-(5-methylsulfonyl-3- | | | | |
| | pyridyl)-5- | | | | |
| | (trifluoromethyl)tetrahydrofuran-2- | | | | |
| | carboxamide | | | | |
| 668 | (2R,3S,4S,5R)-3-[3,4-difluoro-2- | A | 483.38 | 484.4 | 3.07 |
| | (hydroxymethyl)phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 669 | (2R,3S,4S,5R)-N-[1-(difluoromethyl)- | A | 580.538 | 581.6 | 3.7 |
| | 3-methyl-pyrazol-4-yl]-3-[3,4- | | | | |
| | difluoro-2-[methyl(tetrahydropyran- | | | | |
| | 4-yl)amino]methyl]phenyl]-4,5- | | | | |
| | dimethyl-5- | | | | |
| | (trifluoromethyl)tetrahydrofuran-2- | | | | |
| | carboxamide | | | | |
| 670 | (2R,3S,4S,5R)-N-[1-(difluoromethyl)- | A | 564.496 | 565.6 | 3.37 |
| | 3-methyl-pyrazol-4-yl]-3-[3,4- | | | | |
| | difluoro-2-(2-oxa-6- | | | | |
| | azaspiro [3.3]heptan-6- | | | | |
| | ylmethyl)phenyl]-4,5-dimethyl-5- | | | | |
| | (trifluoromethyl)tetrahydrofuran-2- | | | | |
| | carboxamide | | | | |
| 671 | (2R,3S,4S,5R)-3-[3,4-difluoro-2- | A | 496.422 | 497.5 | 3.29 |
| | (methylaminomethyl)phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 672 | (2R,3S,4S,5R)-N-[1-(difluoromethyl)- | A [2], A | 510.448 | 511.55 | 3.805 |
| | 3-methyl-pyrazol-4-yl]-3-[2- | | | | |
| | [(dimethylamino)methyl]-3,4- | | | | |
| | difluoro-phenyl]-4,5-dimethyl-5- | | | | |
| | (trifluoromethyl)tetrahydrofuran-2- | | | | |
| | carboxamide | | | | |
| 673 | (2R,3S,4S,5R)-3-[3,4-difluoro-2-[(3- | C | 538.458 | 539.3 | 0.87 |
| | hydroxyazetidin-1-yl)methyl]phenyl]- | | | | |
| | N-[1-(difluoromethyl)-3-methyl- | | | | |
| | pyrazol-4-yl]-4,5-dimethyl-5- | | | | |
| | (trifluoromethyl)tetrahydrofuran-2- | | | | |
| | carboxamide | | | | |
| 674 | (2R,3S,4S,5R)-N-[1-(difluoromethyl)- | A | 565.527 | 567.3 | 3.4 |
| | 3-methyl-pyrazol-4-yl]-3-[3,4- | | | | |
| | difluoro-2-[(4-methylpiperazin-1- | | | | |
| | yl)methyl]phenyl]-4,5-dimethyl-5- | | | | |
| | (trifluoromethyl)tetrahydrofuran-2- | | | | |
| | carboxamide | | | | |
| 675 | (2R,3S,4S,5R)-N-[1-(difluoromethyl)- | A | 524.475 | 525.2 | 3.97 |
| | 3-methyl-pyrazol-4-yl]-3-[2- | | | | |
| | [[ethyl(methyl)amino]methyl]-3,4- | | | | |
| | difluoro-phenyl]-4,5-dimethyl-5- | | | | |
| | (trifluoromethyl)tetrahydrofuran-2- | | | | |
| | carboxamide | | | | |
| 676 | (2R,3S,4S,5R)-N-[1-(difluoromethyl)- | A | 479.391 | 480.4 | 3.65 |
| | 3-methyl-pyrazol-4-yl]-3-(3,4- | | | | |
| | difluoro-2-vinyl-phenyl)-4,5- | | | | |
| | dimethyl-5- | | | | |
| | (trifluoromethyl)tetrahydrofuran-2- | | | | |
| | carboxamide | | | | |
| 677 | (2R,3S,4S,5R)-3-[3,4-difluoro-2-(2- | E | 497.407 | 498.3 | 3.34 |
| | hydroxyethyl)phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 678 | (2R,3S,4S,5R)-N-[1-(difluoromethyl)- | C | 524.475 | 526.3 | 0.97 |
| | 3-methyl-pyrazol-4-yl]-3-[2-[2- | | | | |
| | (dimethylamino)ethyl]-3,4-difluoro- | | | | |
| | phenyl]-4,5-dimethyl-5- | | | | |
| | (trifluoromethyl)tetrahydrofuran-2- | | | | |
| | carboxamide | | | | |
| 679 | (2R,3S,4S,5R)-N-[1-(difluoromethyl)- | C | 578.522 | 579.3 | 3.21 |
| | 3-methyl-pyrazol-4-yl]-3-[3,4- | | | | |
| | difluoro-2-[2-(2-oxa-6- | | | | |
| | azaspiro[3.3]heptan-6- | | | | |
| | yl)ethyl]phenyl]-4,5-dimethyl-5- | | | | |
| | (trifluoromethyl)tetrahydrofuran-2- | | | | |
| | carboxamide | | | | |
| 680 | (2R,3S,4S,5R)-3-[3,4-difluoro-2-(1- | A | 497.407 | 498.4 | 3.14 |
| | hydroxyethyl)phenyl]-N-[1- | | | | |
| | (difluoromethyl)-3-methyl-pyrazol-4- | | | | |
| | yl]-4,5-dimethyl-5- | | | | |
| | (trifluoromethyl)tetrahydrofuran-2- | | | | |
| | carboxamide | | | | |
| 681 | (2R,3S,4S,5R)-N-[1-(difluoromethyl)- | A | 547.468 | 548.3 | 3.26 |
| | 3-methyl-pyrazol-4-yl]-3-[3,4- | | | | |
| | difluoro-2-[2-(1H-pyrazol-4- | | | | |
| | yl)ethyl]phenyl]-4,5-dimethyl-5- | | | | |
| | (trifluoromethyl)tetrahydrofuran-2- | | | | |
| | carboxamide | | | | |
| 682 | 5-[[(2R,3S,4S,5R)-3-[4- | A | 521.41 | 522.1 | 3.28 |
| | (difluoromethoxy)-3-fluoro-2- | | | | |
| | methoxy-phenyl]-4,5-dimethyl-5- | | | | |
| | (trifluoromethyl)tetrahydrofuran-2- | | | | |
| | carbonyl]amino]pyridine-2- | | | | |
| | carboxamide | | | | |
| 683 | 3-(3,4-difluoro-2-methoxy-phenyl)-5- | A | 497.436 | 498.1 | 3.27 |
| | methyl-N-(3-methyl-1- | | | | |
| | methylsulfonyl-pyrazol-4-yl)-5- | | | | |
| | (trifluoromethyl)tetrahydrofuran-2- | | | | |
| | carboxamide | | | | |
| 684 | 3-(3,4-difluoro-2-methoxy-phenyl)-5- | A | 497.436 | 498.1 | 3.28 |
| | methyl-N-(3-methyl-1- | | | | |
| | methylsulfonyl-pyrazol-4-yl)-5- | | | | |
| | (trifluoromethyl)tetrahydrofuran-2- | | | | |
| | carboxamide | | | | |
| 685 | 3-(3,4-difluoro-2-methoxy-phenyl)-N- | A | 476.394 | 477.2 | 2.88 |
| | [2-(1,2-dihydroxyethyl)-4-pyridyl]-5- | | | | |
| | methyl-5- | | | | |
| | (trifluoromethyl)tetrahydrofuran-2- | | | | |
| | carboxamide | | | | |
| 686 | 3-(3,4-difluoro-2-methoxy-phenyl)-N- | A | 476.394 | 476.8 | 2.88 |
| | [2-(1,2-dihydroxyethyl)-4-pyridyl]-5- | | | | |
| | methyl-5- | | | | |
| | (trifluoromethyl)tetrahydrofuran-2- | | | | |
| | carboxamide | | | | |
| 687 | 3-(3,4-difluoro-2-methoxy-phenyl)-N- | A | 476.394 | 477 | 2.88 |
| | [2-(1,2-dihydroxyethyl)-4-pyridyl]-5- | | | | |
| | methyl-5- | | | | |
| | (trifluoromethyl)tetrahydrofuran-2- | | | | |
| | carboxamide | | | | |
| 688 | 3-(3,4-difluoro-2-methoxy-phenyl)-N- | A | 476.394 | 476.9 | 2.88 |
| | [2-(1,2-dihydroxyethyl)-4-pyridyl]-5- | | | | |
| | methyl-5- | | | | |
| | (trifluoromethyl)tetrahydrofuran-2- | | | | |
| | carboxamide | | | | |
| 689 | 3-(3,4-difluoro-2-methoxy-phenyl)-N- | A | 494.384 | 494.8 | 3.02 |
| | [2-(1,2-dihydroxyethyl)-5-fluoro-4- | | | | |
| | pyridyl]-5-methyl-5- | | | | |
| | (trifluoromethyl)tetrahydrofuran-2- | | | | |
| | carboxamide | | | | |
| 690 | 3-(3,4-difluoro-2-methoxy-phenyl)-N- | A | 494.384 | 494.8 | 3.02 |
| | [2-(1,2-dihydroxyethyl)-5-fluoro-4- | | | | |
| | pyridyl]-5-methyl-5- | | | | |
| | (trifluoromethyl)tetrahydrofuran-2- | | | | |
| | carboxamide | | | | |
| 691 | 3-(3,4-difluoro-2-methoxy-phenyl)-N- | A | 494.384 | 494.9 | 3.02 |
| | [2-(1,2-dihydroxyethyl)-5-fluoro-4- | | | | |
| | pyridyl]-5-methyl-5- | | | | |
| | (trifluoromethyl)tetrahydrofuran-2- | | | | |
| | carboxamide | | | | |
| 692 | 3-(3,4-difluoro-2-methoxy-phenyl)-N- | A | 494.384 | 494.9 | 3.02 |
| | [2-(1,2-dihydroxyethyl)-5-fluoro-4- | | | | |
| | pyridyl]-5-methyl-5- | | | | |
| | (trifluoromethyl)tetrahydrofuran-2- | | | | |
| | carboxamide | | | | |
| 693 | 3-(3,4-difluoro-2-methoxy-phenyl)-N- | A | 490.42 | 491 | 2.98 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 694 | 3-(3,4-difluoro-2-methoxy-phenyl)-N- | A | 490.42 | 490.9 | 2.97 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 695 | 3-(3,4-difluoro-2-methoxy-phenyl)-N- | A | 490.42 | 491 | 2.97 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 696 | 3-(3,4-difluoro-2-methoxy-phenyl)-N- | A | 490.42 | 491 | 2.97 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 697 | 3-(3,4-difluoro-2-methoxy-phenyl)-N- | A | 490.42 | 491 | 2.96 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 698 | 3-(3,4-difluoro-2-methoxy-phenyl)-N- | A | 490.42 | 491 | 2.96 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 699 | 3-(3,4-difluoro-2-methoxy-phenyl)-N- | A | 490.42 | 491 | 2.96 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 700 | 3-(3,4-difluoro-2-methoxy-phenyl)-N- | A | 490.42 | 491 | 2.96 |
| | [2-(1,2-dihydroxypropyl)-4-pyridyl]- | | | | |
| | 5-methyl-5- | | | | |
| | (trifluoromethyl)tetrahydrofuran-2- | | | | |
| | carboxamide | | | | |
| 701 | 3-(3,4-difluoro-2-methoxy-phenyl)-N- | A | 490.42 | 491.5 | 95.9 |
| | [2-(1,2-dihydroxy-1-methyl-ethyl)-4- | | | | |
| | pyridyl]-5-methyl-5- | | | | |
| | (trifluoromethyl)tetrahydrofuran-2- | | | | |
| | carboxamide | | | | |
| 702 | 3-(3,4-difluoro-2-methoxy-phenyl)-N- | A | 490.42 | 491.5 | 2.95 |
| | [2-(1,2-dihydroxy-1-methyl-ethyl)-4- | | | | |
| | pyridyl]-5-methyl-5- | | | | |
| | (trifluoromethyl)tetrahydrofuran-2- | | | | |
| | carboxamide | | | | |
| 703 | N-[2-(1,2-dihydroxyethyl)-4-pyridyl]- | A | 472.43 | 473.5 | 2.87 |
| | 3-(4-fluoro-2-methoxy-3-methyl- | | | | |
| | phenyl)-5-methyl-5- | | | | |
| | (trifluoromethyl)tetrahydrofuran-2- | | | | |
| | carboxamide | | | | |
| 704 | N-[2-(1,2-dihydroxyethyl)-4-pyridyl]- | A | 472.43 | 473.5 | 2.87 |
| | 3-(4-fluoro-2-methoxy-3-methyl- | | | | |
| | phenyl)-5-methyl-5- | | | | |
| | (trifluoromethyl)tetrahydrofuran-2- | | | | |
| | carboxamide | | | | |
| 705 | N-[2-(1,2-dihydroxyethyl)-4-pyridyl]- | A | 472.43 | 473.5 | 2.87 |
| | 3-(4-fluoro-2-methoxy-3-methyl- | | | | |
| | phenyl)-5-methyl-5- | | | | |
| | (trifluoromethyl)tetrahydrofuran-2- | | | | |
| | carboxamide | | | | |
| 706 | N-[2-(1,2-dihydroxyethyl)-4-pyridyl]- | A | 472.43 | 473.5 | 2.87 |
| | 3-(4-fluoro-2-methoxy-3-methyl- | | | | |
| | phenyl)-5-methyl-5- | | | | |
| | (trifluoromethyl)tetrahydrofuran-2- | | | | |
| | carboxamide | | | | |
| 707 | 3-(2-ethoxy-3,4-difluoro-phenyl)-N- | A | 500.419 | | 2.94 |
| | (3-methoxy-[1,2,4]triazolo[4,3- | | | | |
| | alpyridin-7-yl)-5-methyl-5- | | | | |
| | (trifluoromethyl)tetrahydrofuran-2- | | | | |
| | carboxamide | | | | |
| 708 | 3-(2-ethoxy-3,4-difluoro-phenyl)-N- | A | 500.419 | 501.3 | 2.94 |
| | (3-methoxy-[1,2,4]triazolo[4,3- | | | | |
| | alpyridin-7-yl)-5-methyl-5- | | | | |
| | (trifluoromethyl)tetrahydrofuran-2- | | | | |
| | carboxamide | | | | |
| 709 | N-[2-(1,2-dihydroxyethyl)-4-pyridyl]- | A | 490.42 | 491.5 | 2.98 |
| | 3-(2-ethoxy-3,4-difluoro-phenyl)-5- | | | | |
| | methyl-5- | | | | |
| | (trifluoromethyl)tetrahydrofuran-2- | | | | |
| | carboxamide | | | | |
| 710 | N-[2-(1,2-dihydroxyethyl)-4-pyridyl]- | A | 490.42 | 491.4 | 2.98 |
| | 3-(2-ethoxy-3,4-difluoro-phenyl)-5- | | | | |
| | methyl-5- | | | | |
| | (trifluoromethyl)tetrahydrofuran-2- | | | | |
| | carboxamide | | | | |
| 711 | N-[2-(1,2-dihydroxyethyl)-4-pyridyl]- | A | 490.42 | 491.3 | 2.96 |
| | 3-(2-ethoxy-3,4-difluoro-phenyl)-5- | | | | |
| | methyl-5- | | | | |
| | (trifluoromethyl)tetrahydrofuran-2- | | | | |
| | carboxamide | | | | |
| 712 | N-[2-(1,2-dihydroxyethyl)-4-pyridyl]- | A | 490.42 | 491.6 | 2.98 |
| | 3-(2-ethoxy-3,4-difluoro-phenyl)-5- | | | | |
| | methyl-5- | | | | |
| | (trifluoromethyl)tetrahydrofuran-2- | | | | |
| | carboxamide | | | | |
| 713 | 3-(4-fluoro-2-methoxy-phenyl)-5- | A | 476.442 | 477.6 | 3.13 |
| | methyl-N-(2-methylsulfonyl-4- | | | | |
| | pyridyl)-5- | | | | |
| | (trifluoromethyl)tetrahydrofuran-2- | | | | |
| | carboxamide | | | | |
| 714 | 3-(4-fluoro-2-methoxy-phenyl)-5- | A | 476.442 | 477.6 | 3.13 |
| | methyl-N-(2-methylsulfonyl-4- | | | | |
| | pyridyl)-5- | | | | |
| | (trifluoromethyl)tetrahydrofuran-2- | | | | |
| | carboxamide | | | | |
| 715 | 3-(4-fluoro-2-methoxy-phenyl)-5- | A | 475.457 | 476.1 | 2.86 |
| | methyl-N-[2-(methylsulfonimidoyl)- | | | | |
| | 4-pyridyl]-5- | | | | |
| | (trifluoromethyl)tetrahydrofuran-2- | | | | |
| | carboxamide | | | | |
| 716 | 3-(3-ethyl-4-fluoro-2-methoxy- | A | 507.499 | 508.2 | 3.47 |
| | phenyl)-5-methyl-N-(3-methyl-1- | | | | |
| | methylsulfonyl-pyrazol-4-yl)-5- | | | | |
| | (trifluoromethyl)tetrahydrofuran-2- | | | | |
| | carboxamide | | | | |
| 717 | 3-(3-ethyl-4-fluoro-2-methoxy- | A | 507.499 | 508.2 | 3.47 |
| | phenyl)-5-methyl-N-(3-methyl-1- | | | | |
| | methylsulfonyl-pyrazol-4-yl)-5- | | | | |
| | (trifluoromethyl)tetrahydrofuran-2- | | | | |
| | carboxamide | | | | |
| 718 | 3-(3,4-difluoro-2-methoxy-phenyl)-N- | A | 422.422 | 423.5 | 2.62 |
| | [2-(1,2-dihydroxyethyl)-4-pyridyl]- | | | | |
| | 4,5-dimethyl-tetrahydrofuran-2- | | | | |
| | carboxamide | | | | |
| 719 | 3-(3,4-difluoro-2-methoxy-phenyl)-N- | A | 422.422 | 423.5 | 2.62 |
| | [2-(1,2-dihydroxyethyl)-4-pyridyl]- | | | | |
| | 4,5-dimethyl-tetrahydrofuran-2- | | | | |
| | carboxamide | | | | |
| 720 | (2R,3S,4S,5S)-3-(3,4-difluoro-2- | A | 390.424 | 391.6 | 3.34 |
| | methoxy-phenyl)-5-isopropyl-4- | | | | |
| | methyl-N-(3-pyridyl)tetrahydrofuran- | | | | |
| | 2-carboxamide | | | | |
| 721 | 3-(3,4-difluoro-2-methoxy-phenyl)-N- | A | 436.449 | 437 | 2.83 |
| | [2-(1,2-dihydroxyethyl)-4-pyridyl]- | | | | |
| | 4,5,5-trimethyl-tetrahydrofuran-2- | | | | |
| | carboxamide | | | | |
| 722 | 3-(3,4-difluoro-2-methoxy-phenyl)-N- | A | 436.449 | 437 | 2.8 |
| | [2-(1,2-dihydroxyethyl)-4-pyridyl]- | | | | |
| | 4,5,5-trimethyl-tetrahydrofuran-2- | | | | |
| | carboxamide | | | | |
| 723 | 3-(3,4-difluoro-2-methoxy-phenyl)-N- | A | 436.449 | 437 | 2.79 |
| | [2-(1,2-dihydroxyethyl)-4-pyridyl]- | | | | |
| | 4,5,5-trimethyl-tetrahydrofuran-2- | | | | |
| | carboxamide | | | | |
| 724 | 3-(3,4-difluoro-2-methoxy-phenyl)-N- | A | 436.449 | 437 | 2.82 |
| | [2-(1,2-dihydroxyethyl)-4-pyridyl]- | | | | |
| | 4,5,5-trimethyl-tetrahydrofuran-2- | | | | |
| | carboxamide | | | | |
| 725 | 3-(3,4-difluoro-2-methoxy-phenyl)-N- | A | 450.476 | 451.4 | 2.86 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 726 | 3-(3,4-difluoro-2-methoxy-phenyl)-N- | A | 450.476 | 451.4 | 2.86 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 727 | 3-(3,4-difluoro-2-methoxy-phenyl)-N- | A | 450.476 | 451.4 | 2.89 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 728 | 3-(3,4-difluoro-2-methoxy-phenyl)-N- | A | 450.476 | 451.4 | 2.89 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 729 | 3-(3,4-difluoro-2-methoxy-phenyl)-N- | A | 450.476 | 451.4 | 2.86 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 730 | 3-(3,4-difluoro-2-methoxy-phenyl)-N- | A | 450.476 | 451.4 | 2.85 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 731 | 3-(3,4-difluoro-2-methoxy-phenyl)-N- | A | 450.476 | 451.4 | 2.88 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 732 | 3-(3,4-difluoro-2-methoxy-phenyl)-N- | A | 450.476 | 451.4 | 2.88 |
| | [2-(1,2-dihydroxy-1-methyl-ethyl)-4- | | | | |
| | pyridyl]-4,5,5-trimethyl- | | | | |
| | tetrahydrofuran-2-carboxamide | | | | |
| 733 | 5-((2R,3S,4S,5R)-3-(3,4-difluoro-5- | A | 489.1 | 490.4 | 2.75 |
| | hydroxy-2-methoxyphenyl)-4,5- | | | | |
| | dimethyl-5- | | | | |
| | (trifluoromethyl)tetrahydrofuran-2- | | | | |
| | carboxamido)picolinamide | | | | |
| 734 | rel-5-((2R,3S,4S,5R)-3-(6- | A | 488.1 | 489.3 | 3.22 |
| | (difluoromethyl)-2-methoxypyridin-3- | | | | |
| | yl)-4,5-dimethyl-5- | | | | |
| | (trifluoromethyl)tetrahydrofuran-2- | | | | |
| | carboxamido)picolinamide | | | | |
| 735 | rel-5-((2S,3R,4R,5S)- 3-(6- | A | 488.1 | 489.3 | 3.22 |
| | (difluoromethyl)-2-methoxypyridin-3- | | | | |
| | yl)-4,5-dimethyl-5- | | | | |
| | (trifluoromethyl)tetrahydrofuran-2- | | | | |
| | carboxamido)picolinamide | | | | |

### Intermediates:

### Intermediate A

### 2-(1-methyl-1-trimethylsilyloxy-ethyl)pyridin-4-amine

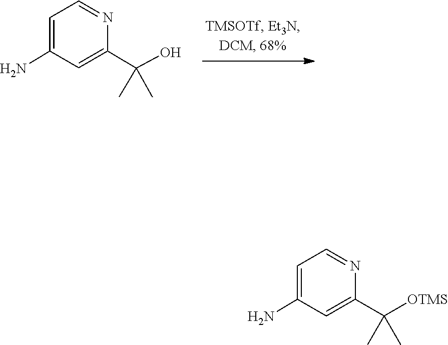

To a solution of 2-(4-amino-2-pyridyl)propan-2-ol (2 g, 13.14 mmol) and Et₃N (5.5 mL, 39.46 mmol) in DCM (20 mL) was added trimethylsilyl trifluoromethanesulfonate (7.1 mL, 39.29 mmol). The reaction was stirred at ambient temperature for 20 min, then cooled to 0° C. and saturated aqueous sodium bicarbonate solution (10 mL) and water (5 mL) were added. The organic layer was separated and the aqueous layer was extracted with DCM (2×10 mL). The combined organic extracts were dried (MgSO₄) and concentrated in vacuo to afford 2-(1-methyl-1-trimethylsilyloxy-ethyl)pyridin-4-amine (2 g at 91% purity, 68%). ESI-MS m/z calc. 224.13449, found 225.6 (M+1)⁺.

### Intermediate B

### 1-[[(4R)-2,2-dimethyl-1,3-dioxolan-4-yl]methyl]-3-methyl-pyrazol-4-amine

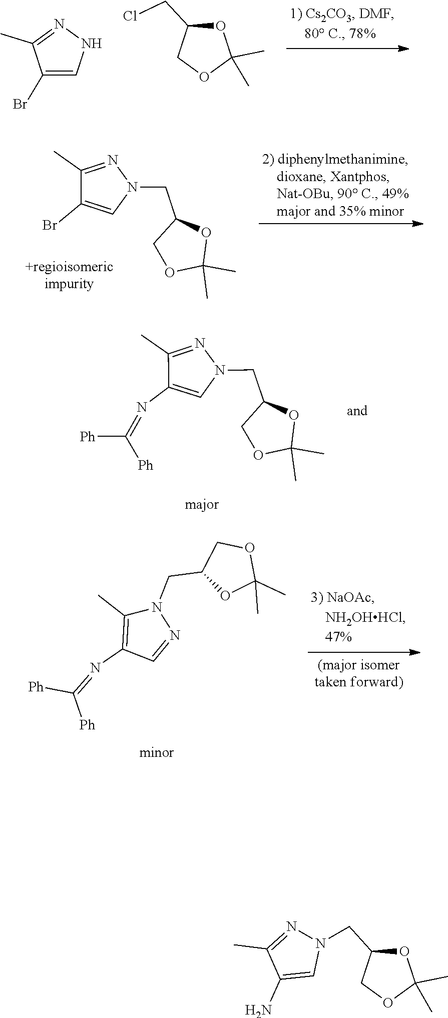

### Step 1:

(4S)-4-(chloromethyl)-2,2-dimethyl-1,3-dioxolane (8.99 g, 59.69 mmol) was added to a mixture of 4-bromo-3-methyl-1H-pyrazole (9 g) TBDMS, 55.90 mmol) and cesium carbonate (20 g, 61.38 mmol) in DMF (130 mL) and the reaction was heated at 80° C. for 18 h. The reaction mixture was concentrated in vacuo and EtOAc (400 mL) was added. The mixture was filtered through cotton wool and the filtrate was washed with water (4×80 mL), brine (150 mL), dried (Na₂SO₄), filtered and concentrated in vacuo. Purification by flash chromatography (220 g, SiO₂, 0-90% EtOAc in heptane) gave 4-bromo-1-[[(4R)-2,2-dimethyl-1,3-dioxolan-4-yl]methyl]-3-methyl-pyrazole (11.96 g, 78%). ¹H NMR (300 MHz, Chloroform-d) δ 7.45-7.40 (m, 1H), 4.49-4.35 (m, 1H), 4.22-4.02 (m, 3H), 3.89-3.69 (m, 1H), 2.34-2.19 (m, 3H), 1.42-1.31 (m, 6H) ppm; ESI-MS m/z calc. 274.0317, found 275.1 (M+1)⁺.

### Step 2:

A solution of 4-bromo-1-[[(4R)-2,2-dimethyl-1,3-dioxolan-4-yl]methyl]-3-methyl-pyrazole (11.96 g, 43.47 mmol) and diphenylmethanimine (8.7480 g, 8.1 mL, 48.27 mmol) in 1,4-dioxane (130 mL) was degassed with nitrogen for 20 min. Xantphos (2.6 g, 4.49 mmol), tris(dibenzylideneacetone)dipalladium(0) (2 g, 2.18 mmol) and sodium tert-butoxide (12.5 g, 130.07 mmol) were added and the resulting mixture was stirred at 90° C. for 4 h. The reaction mixture was concentrated in vacuo and the residue was partitioned between saturated aqueous NH₄Cl solution (250 mL) and EtOAc (300 mL). The aqueous layer was extracted with EtOAc (100 mL) and the combined organic extracts were washed with water (200 mL), brine (2×150 mL), dried (Na₂SO₄), filtered and concentrated in vacuo. Purification by flash chromatography (330 g SiO₂, 0 to 50% EtOAc in heptane) gave N-[1-[[(4R)-2,2-dimethyl-1,3-dioxolan-4-yl]methyl]-3-methyl-pyrazol-4-yl]-1,1-diphenyl-methanimine (8 g, 49%) as a yellow oil. ¹H NMR (300 MHz, Chloroform-d) δ 7.83-7.74 (m, 2H), 7.54-7.45 (m, 3H), 7.43-7.32 (m, 3H), 7.25-7.18 (m, 2H), 5.86 (s, 1H), 4.24 (quin, J=5.9 Hz, 1H), 3.93 (dd, J=8.5, 6.2 Hz, 1H), 3.88 (d, J=5.9 Hz, 2H), 3.59 (dd, J=8.8, 5.9 Hz, 1H), 2.39 (s, 3H), 1.29 (s, 6H) ppm; ESI-MS m/z calc. 375.1947, found 376.2 (M+1).

and

N-[1-[[(4R)-2,2-dimethyl-1,3-dioxolan-4-yl]methyl]-5-methyl-pyrazol-4-yl]-1,1-diphenyl-methanimine (5.68 g, 35%) as a yellow oil. ¹H NMR (300 MHz, Chloroform-d) δ 7.83-7.73 (m, 2H), 7.54-7.44 (m, 3H), 7.42-7.31 (m, 3H), 7.24-7.17 (m, 2H), 5.99 (s, 1H), 4.40 (quin, J=5.8 Hz, 1H), 4.19-3.99 (m, 3H), 3.87-3.77 (m, 1H), 2.49 (s, 3H), 1.36 (s, 3H), 1.33 (s, 3H) ppm; ESI-MS m/z calc. 375.1947, found 376.2 (M+1)⁺.

### Step 3:

To a solution of N-[1-[[(4R)-2,2-dimethyl-1,3-dioxolan-4-yl]methyl]-3-methyl-pyrazol-4-yl]-1,1-diphenyl-methanimine (8 g, 21.31 mmol) in MeOH (240 mL) at ambient temperature was added NaOAc (7 g, 85.33 mmol) and hydroxylamine hydrochloride (4.5 g, 64.76 mmol). The resulting mixture was stirred at ambient temperature for 1.5 h before the solvent volume was reduced by 50% in vacuo. Saturated aqueous NaHCO₃ (250 mL) was added and the mixture extracted with EtOAc (2 x, 400 mL then 150 mL). The combined organic extracts were washed with brine (200 mL), dried (Na₂SO₄), filtered and concentrated in vacuo. The residue was adsorbed on silica gel and purified by flash chromatography (220 g SiO₂, 0 to 15% MeOH in DCM) to give 1-[[(4R)-2,2-dimethyl-1,3-dioxolan-4-yl]methyl]-3-methyl-pyrazol-4-amine (2.17 g, 47%) as a brown oil. ¹H NMR (300 MHz, Chloroform-d) δ 7.02 (s, 1H), 4.38 (quin, J=5.9 Hz, 1H), 4.08-3.98 (m, 3H), 3.70 (dd, J=8.7, 6.0 Hz, 1H), 2.61 (br. s., 2H), 2.15 (s, 3H), 1.39 (s, 3H), 1.33 (s, 3H) ppm; ESI-MS m/z calc. 211.1321, found 212.2 (M+1).

### Intermediate C

### tert-butyl N-[(4-amino-5-fluoro-2-pyridyl)methyl]-N-methyl-carbamate

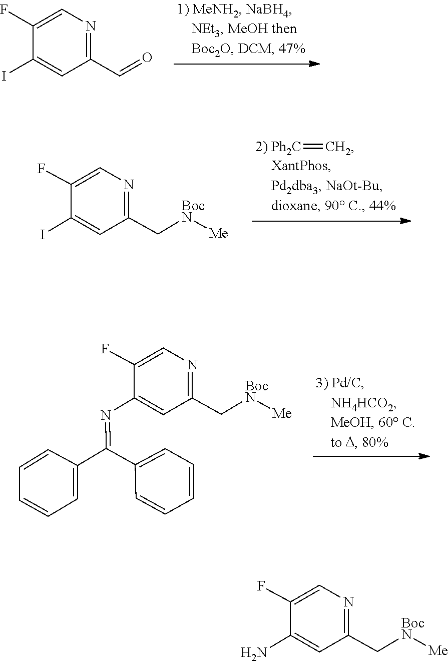

### Step 1:

A solution of methanamine (23 mL of 2 M, 46.00 mmol) in THF was added to a stirred solution of 5-fluoro-4-iodo-pyridine-2-carbaldehyde (7.61 g, 30.32 mmol) in MeOH (100 mL) and the mixture stirred at ambient temperature for 2.5 h. A further portion of methanamine (10 mL of 2 M, 20.00 mmol) was added and the reaction stirred at ambient temperature for 2 h. NaBH₄ (700 mg, 18.50 mmol) was added, portionwise, and the mixture stirred at ambient temperature for 45 min and then concentrated in vacuo. The residue was dissolved in DCM (200 mL) followed by the addition of Et₃N (5 mL, 35.87 mmol) and tert-butoxycarbonyl tert-butyl carbonate (7.9 g, 36.20 mmol). The reaction mixture was stirred at ambient temperature for 16 h and then concentrated in vacuo. Purification by flash chromatography (220 g SiO₂, 0 to 20% EtOAc in heptane) gave tert-butyl N-[(5-fluoro-4-iodo-2-pyridyl)methyl]-N-methyl-carbamate (5.2033 g, 47%) as a pale yellow oil. ¹H NMR (500 MHz, DMSO-d₆) δ 8.44 (s, 1H), 7.71 (d, J=4.9 Hz, 1H), 4.40 (s, 2H), 2.86 (d, J=4.7 Hz, 3H), 1.43 0 1.30 (m, 9H) ppm; ¹⁹F NMR (471 MHz, DMSO-d₆) δ−114.20, −114.38 ppm; ESI-MS m/z calc. 366.02405, found 310.9 (M-tBu)⁺.

### Step 2:

To a solution of tert-butyl N-[(5-fluoro-4-iodo-2-pyridyl)methyl]-N-methyl-carbamate (5.20 g, 14.20 mmol) and Xantphos (850 mg, 1.469 mmol) in 1,4-dioxane (100 mL) was added Pd₂dba₃ (670 mg, 0.73 mmol). After stirring for 5 min under a nitrogen atmosphere diphenylmethanimine (2.4 mL, 14.30 mmol) and NaOt-Bu (4.12 g, 42.87 mmol) were added. The mixture was degassed with nitrogen and then heated at 90° C. for 18 h. The mixture was partitioned between EtOAc (40 mL) and water (40 mL) and filtered. The aqueous layer was extracted with EtOAc (2×20 mL). The combined organic extracts were dried (MgSO₄), filtered and concentrated in vacuo. Purification by flash chromatography (220 g SiO₂, 0 to 20% then 100% EtOAc in heptane, loaded in DCM) gave tert-butyl N-[[4-(benzhydrylideneamino)-5-fluoro-2-pyridyl]methyl]-N-methyl-carbamate (2.6327 g, 44%) as a brown oil. H NMR (500 MHz, DMSO-d₆) δ 8.28 (d, J=2.0 Hz, 1H), 7.72-7.18 (m, 10H), 6.65 (s, 1H), 4.29 (s, 2H), 2.70-2.63 (m, 3H), 1.42-1.30 (d, J=60.6 Hz, 9H) ppm; ¹⁹F NMR (471 MHz, DMSO-d₆) δ−144.73; ESI-MS m/z calc. 419.2009, found 420.2 (M+1).

### Step 3:

Pd/C (wet, Degussa, 675 mg of 10% w/w, 0.63 mmol) was added to a stirred solution of tert-butyl N-[[4-(benzhydrylideneamino)-5-fluoro-2-pyridyl]methyl]-N-methyl-carbamate (2.63 g, 6.269 mmol) and ammonium formate (8.0 g, 126.9 mmol) in MeOH (35 mL) and the mixture heated at 60° C. for 18 h, then at reflux for 48 h. The solution was cooled to ambient temperature, diluted with DCM (100 mL) and filtered through a pad of celite. The filtrate was washed with 0.1M NaOH (25 mL), dried (MgSO₄), filtered and concentrated in vacuo. Purification by flash chromatography (40 g SiO₂, 0 to 100% EtOAc in petroleum ether) gave tert-butyl N-[(4-amino-5-fluoro-2-pyridyl)methyl]-N-methyl-carbamate (1.2751 g, 80%) as an orange oil. ¹H NMR (500 MHz, DMSO-d₆) δ 8.00 (d, J=3.5 Hz, 1H), 6.53 (d, J=7.4 Hz, 1H), 6.19 (s, 2H), 4.23 (s, 2H), 2.81 (s, 3H), 1.44-1.35 (m, 9H) ppm; ¹⁹F NMR (471 MHz, DMSO-d₆) δ−155.10, −155.23 ppm; ESI-MS m/z calc. 255.1383, found 200.0 (M-tBu)⁺.

### Intermediate D

### tert-butyl N-[(4-amino-2-pyridyl)methyl]-N-(2-methoxyethyl)carbamate

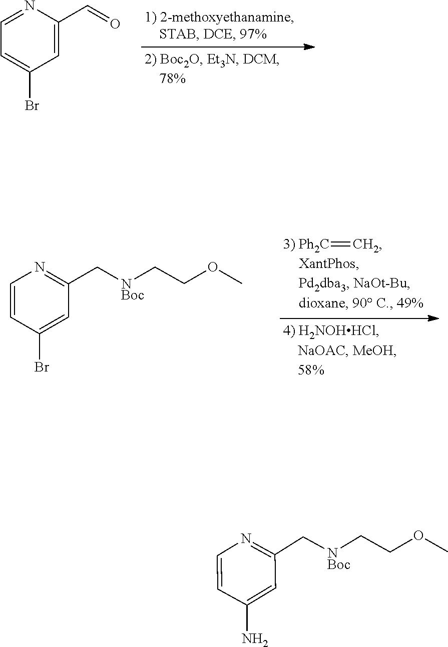

### Step 1:

To a solution of 4-bromopyridine-2-carbaldehyde (2.512 g, 13.50 mmol) in DCE (60 mL) at ambient temperature was added 2-methoxyethanamine (1.4 mL, 16.10 mmol). When complete conversion to the imine intermediate had occurred sodium triacetoxyborohydride (5.697 g, 27.01 mmol) was added. The reaction was stirred for 16 h at ambient temperature. Saturated sodium bicarbonate was added and the mixture was extracted with DCM. The organic extract was dried (MgSO₄) and concentrated in vacuo to give N-[(4-bromo-2-pyridyl)methyl]-2-methoxy-ethanamine (3.22 g, 97%). ¹H NMR (500 MHz, DMSO-d₆) δ 8.38 (dd, J=5.4, 0.5 Hz, 1H), 7.70 (dd, J=2.0, 0.7 Hz, 1H), 7.52 (dd, J=5.3, 2.0 Hz, 1H), 3.81 (s, 2H), 3.40 (t, J=5.6 Hz, 2H), 3.24 (s, 3H), 2.67 (t, J=5.6 Hz, 2H) ppm; ESI-MS m/z calc. 244.02112, found 247.5 (M+1).

### Step 2:

To a solution of N-[(4-bromo-2-pyridyl)methyl]-2-methoxy-ethanamine (3.215 g, 13.12 mmol) in DCM (90 mL) at 0° C. was added Et₃N (2.2 mL, 15.78 mmol) and di-tert-butyl dicarbonate (3.6 mL, 15.67 mmol). The reaction stirred at ambient temperature for 2 h and then diluted with DCM and washed with water. The organic extract was dried (MgSO₄) and concentrated in vacuo. Purification by flash chromatography (SiO₂, 0-40% EtOAc in heptane) gave tert-butyl N-[(4-bromo-2-pyridyl)methyl]-N-(2-methoxyethyl)carbamate (3.550 g, 78%) ¹H NMR (500 MHz, DMSO-d₆) δ 8.40 (dd, J=5.3, 0.6 Hz, 1H), 7.56 (dd, J=5.3, 1.9 Hz, 1H), 7.45 (d, J=12.1 Hz, 1H), 4.50-4.44 (m, 2H), 3.55-3.38 (m, 4H), 3.22 (s, 3H), 1.43 (s, 4H), 1.24 (s, 5H) ppm; ESI-MS m/z calc. 344.07355, found 347.5 (M+1).

### Step 3:

To a solution of tert-butyl N-[(4-bromo-2-pyridyl)methyl]-N-(2-methoxyethyl)carbamate (2 g, 5.793 mmol) and Xantphos (349.2 mg, 0.60 mmol) in 1,4-dioxane (40 mL) was added Pd₂dba₃ (278.9 mg, 0.30 mmol). The mixture was stirred under nitrogen for 5 min followed by the addition of diphenylmethanimine (1.05 g, 5.79 mmol) and NaOtBu (1.721 g, 17.91 mmol). The reaction mixture was degassed with nitrogen and heated at 90° C. for 18 h. The reaction mixture was cooled to ambient temperature, diluted with water and extracted with EtOAc. The aqueous layer was extracted with EtOAc and the combined organic extracts were dried (MgSO₄), filtered and concentrated in vacuo. Purification by flash chromatography (80 g SiO₂, 0 to 70% EtOAc in heptane) gave tert-butyl N-[[4-(benzhydrylideneamino)-2-pyridyl]methyl]-N-(2-methoxyethyl)carbamate (1.259 g, 49%). ¹H NMR (500 MHz, DMSO-d₆) δ 8.21 (d, J=5.3 Hz, 1H), 7.67 (s, 2H), 7.60-7.54 (m, 1H), 7.50 (s, 2H), 7.35 (s, 3H), 7.17 (s, 2H), 6.64 (d, J=5.1 Hz, 1H), 6.48 (d, J=12.4 Hz, 1H), 4.33 (s, 2H), 3.33 (s, 2H), 3.26 (s, 1H), 3.20 (s, 3H), 3.12 (s, 1H), 1.42 (s, 3H), 1.26 (d, J=10.8 Hz, 6H) ppm; ESI-MS m/z calc. 445.23654, found 446.7 (M+1)⁺.

### Step 4:

To a solution of tert-butyl N-[[4-(benzhydrylideneamino)-2-pyridyl]methyl]-N-(2-methoxyethyl)carbamate (1.257 g, 2.82 mmol) in MeOH (24 mL) was added hydroxylamine hydrochloride (358 mg, 5.15 mmol) and NaOAc (577 mg, 7.03 mmol). The reaction was stirred at ambient temperature for 72 h and then concentrated in vacuo. The residue was passed through an SCX-2 cartridge, washing with MeOH and eluting the product with methanolic ammonia to give tert-butyl N-[(4-amino-2-pyridyl)methyl]-N-(2-methoxyethyl)carbamate (463 mg, 58%). ESI-MS m/z calc. 281.17395, found 282.7 (M+1).

### Intermediate E

### 4-bromo-N,N-dimethyl-pyridine-2-sulfonamide

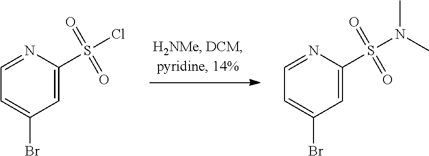

Dimethylamine (975 μL of 2 M, 1.95 mmol) was added to a solution of 4-bromopyridine-2-sulfonyl chloride (500 mg, 1.95 mmol) and pyridine (315 μL, 3.90 mmol) i n DCM (2 mL) stirring at 0° C. The reaction was warmed to ambient temperature and stirred for 1 hour before being quenched with brine and extracted with DCM (×2). The combined organics were dried (MgSO₄) filtered and concentrated in vacuo. The residue was left to stand for three days and the formed yellow crystals were isolated by washing with heptane to give 4-bromo-N,N-dimethyl-pyridine-2-sulfonamide (80 mg, 14%). ¹H NMR (500 MHz, Methanol-d₄) δ 8.56 (dd, J=5.2, 0.6 Hz, 1H), 8.13 (dd, J=1.9, 0.6 Hz, 1H), 7.87 (dd, J=5.2, 1.9 Hz, 1H), 2.90 (s, 6H) ppm; ESI-MS m/z calc. 263.95682, found 265.3 (M+1)⁺.

### Intermediate F

### 4-bromo-N-methyl-pyridine-2-sulfonamide

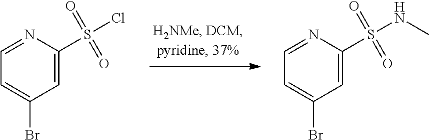

To a solution of 4-bromopyridine-2-sulfonyl chloride (400 mg, 1.56 mmol) and pyridine (252 μL, 3.12 mmol) in DCM at 0° C. was added methylamine (780 μL of 2 M solution in THF, 1.56 mmol). The reaction was stirred at ambient temperature for 2 h and then filtered. The filtrate was partitioned between DCM and brine, the layers separated and the aqueous layer further extracted with DCM (×2). The combined organic extracts were dried (MgSO₄), filtered and concentrated in vacuo. Trituration with heptane gave 4-bromo-N-methyl-pyridine-2-sulfonamide (145 mg, 37%). ¹H NMR (500 MHz, Methanol-d₄) δ 8.55 (dd, J=5.2, 0.6 Hz, 1H), 8.15 (dd, J=1.9, 0.6 Hz, 1H), 7.84 (dd, J=5.2, 1.9 Hz, 1H), 2.68 (s, 3H) ppm; ESI-MS m/z calc. 249.94116, found 251.3 (M+1)⁺; 249.3 (M−1)⁻.

### Intermediate G

### 6-(2-methoxyethoxy)pyridazin-4-amine

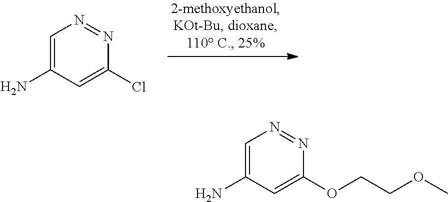

2-Methoxyethanol (420 μL, 5.33 mmol) was added dropwise to a suspension of KOt-Bu (1003 mg, 8.94 mmol) in 1,4-dioxane (13 mL) at 0° C. and the mixture was stirred at 25° C. for 1 hour. 6-chloropyridazin-4-amine (392 mg, 3.03 mmol) was added portionwise and the reaction mixture was stirred at 110° C. overnight. The reaction mixture was poured onto ice cold water (20 mL) and the aqueous layer extracted with EtOAc. The combined organic extracts were washed with brine, dried (MgSO₄) and concentrated in vacuo. The aqueous layer was concentrated in vacuo and combined with organics. Purification by flash chromatography (80 g Al₂O3, 50 to 100% EtOAc in petroleum ether) gave 6-(2-methoxyethoxy)pyridazin-4-amine (125.2 mg, 24%). ¹H NMR (500 MHz, DMSO-d₆) 8.27 (d, J=2.3 Hz, 1H), 6.34 (s, 2H), 5.98 (d, J=2.3 Hz, 1H), 4.46-4.37 (m, 2H), 3.70-3.60 (m, 2H), 3.31 (s, 3H) ppm; ESI-MS m/z calc. 169.08513, found 170.0 (M+1)⁺.

### Intermediate H

### 1-(4-bromo-2-pyridyl)-2-methyl-propan-2-ol

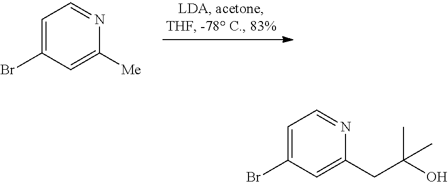

To a solution of 4-bromo-2-methyl-pyridine (801 mg, 4.66 mmol) in THF (10 mL) stirring at −78° C. was added LDA (3.5 mL of 2 M, 7.00 mmol), dropwise. The mixture was stirred at −78° C. for 15 min before acetone (690 μL, 9.397 mmol) was added dropwise. After stirring for a further 45 min at -78° C. the reaction was quenched by addition of saturated aqueous ammonium chloride solution. The aqueous layer was extracted with DCM (3×10 mL) and the combined organic extracts were dried (MgSO₄) and concentrated in vacuo. Purification by flash chromatography (24 g SiO₂, 0 to 100% EtOAc in heptane, loaded in DCM) gave 1-(4-bromo-2-pyridyl)-2-methyl-propan-2-ol (891 mg, 83%). ESI-MS m/z calc. 229.01022, found 232.0 (M+1)⁺.

### Intermediate I

### 3-methoxy-[1,2,4]triazolo[4,3-a]pyridin-7-amine

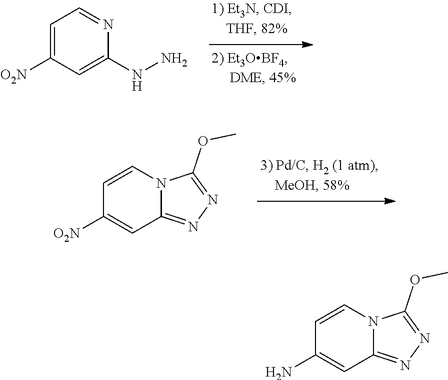

### Step 1:

To a solution of (4-nitro-2-pyridyl)hydrazine dihydrochloride (7.8 g, 30.92 mmol) in THF (350 mL) was added CDI (15.1 g, 93.12 mmol) and Et₃N (21.5 mL, 154.25 mmol). The reaction mixture was stirred at ambient temperature overnight then poured into water (250 mL) and extracted with 3:1 DCM/isopropanol (9×100 mL). The combined organic extracts were dried (Na₂SO₄), filtered and concentrated in vacuo. Purification by flash chromatography (SiO₂, 0 to 10% DCM in MeOH) gave 7-nitro-[1,2,4]triazolo[4,3-a]pyridin-3-ol (4.58 g, 82%) as dark yellow solid. ¹H NMR (250 MHz, DMSO-d₆) δ 8.33 (s, 1H), 8.02 (d, J=7.2 Hz, 1H), 7.11 (dd, J=7.8, 2.2 Hz, 1H) ppm.

### Step 2:

To a solution of 7-nitro-2H-[1,2,4]triazolo[4,3-a]pyridin-3-one (3.01 g, 16.71 mmol) in 1,2-dimethoxyethane (165 mL) was added triethyloxonium tetrafluoroborate (3.7 g, 25.02 mmol). The reaction was stirred at ambient temperature overnight. Saturated aqueous NaHCO₃ (70 mL) was added and the mixture was extracted with 3:1 DCM/iPrOH (9×70 mL). The combined organic extracts were dried (Na₂SO₄) and concentrated in vacuo. Purification by flash chromatography (SiO₂, 0 to 15% DCM in MeOH) gave 3-methoxy-7-nitro-[1,2,4]triazolo[4,3-a]pyridine (1.46 g, 45%) as red solid. ¹H NMR (250 MHz, DMSO-d₆) δ 8.89 (s, 1H), 8.26 (d, J=7.4 Hz, 1H), 7.46 (d, J=8.0 Hz, 1H), 3.96 (s, 3H) ppm; ESI-MS m/z calc. 194.044, found 195.7 (M+1)⁺.

### Step 3:

To a solution of 3-methoxy-7-nitro-[1,2,4]triazolo[4,3-a]pyridine (1.46 g, 7.52 mmol) in MeOH (115 mL) was added 10% Pd/C (405 mg, 3.81 mmol). The reaction mixture was degassed then stirred under a hydrogen atmosphere for 4 h. The reaction mixture was filtered through celite, washing with MeOH (50 mL) and the filtrate was concentrated in vacuo. Purification by flash chromatography (SiO₂, 0 to 25% DCM in MeOH) to give 3-methoxy-[1,2,4]triazolo[4,3-a]pyridin-7-amine (749.4 mg, 58%) as pale orange solid. ¹H NMR (500 MHz, DMSO-d₆) δ 7.76 (dd, J=7.3, 0.6 Hz, 1H), 6.83 (s, 2H), 6.37 (dd, J=7.3, 1.9 Hz, 1H), 6.08 (dd, J=1.9, 0.7 Hz, 1H), 3.45 (s, 3H) ppm; ESI-MS m/z calc. 164.0698, found 165.0 (M+1)⁺.

### Intermediate J

### 2-[2-[tert-butyl(dimethyl)silyl]oxyethylsulfanyl]pyridin-4-amine

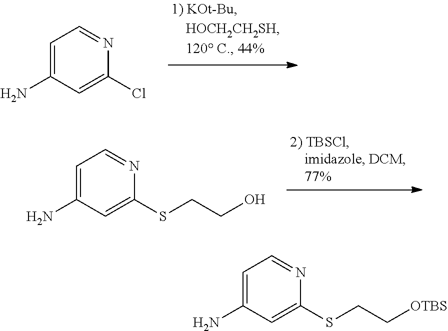 [SMILES: Nc1ccnc(Cl)c1 ; Nc1ccnc(SCCO)c1 ; CC(C)(C)[Si](C)(C)OCCSc1cc(N)ccn1]

### Step 1:

A solution of 2-chloropyridin-4-amine (2 g, 15.56 mmol), KOt-Bu (3.8 g, 33.86 mmol) and 2-sulfanylethanol (1.3 mL, 18.54 mmol) in NMP (20 mL) was heated at 120° C. for 40 h. The reaction was cooled to ambient temperature, further KOt-Bu (3.8 g, 33.86 mmol) and 2-sulfanylethanol (1.3 mL, 18.54 mmol) were added and the reaction heated at 120° C. for 72 h. The reaction mixture was cooled to ambient temperature and concentrated in vacuo. The residue was diluted in DCM (50 mL) and the insoluble material removed by filtration and discarded. Purification of the filtrate by flash chromatography (120 g SiO₂, 0 to 50% 3:1 EtOAc:EtOH in 2% NH₄0H/heptane, loaded in DCM) gave 2-[(4-amino-2-pyridyl)sulfanyl]ethanol (2.306 g at 50% purity, 44%) as an orange oil. ¹H NMR (500 MHz, DMSO-d₆) δ 7.84 (d, J=5.6 Hz, 1H), 6.36 (d, J=2.0 Hz, 1H), 6.25 (dd, J=5.7, 2.2 Hz, 1H), 6.00 (s, 2H), 5.04 (s, 1H), 3.58-3.53 (m, 2H), 3.11 (t, J=6.8 Hz, 2H) ppm; ESI-MS m/z calc. 170.05138, found 171.0 (M+1)⁺.

### Step 2:

Tert-butyl-chloro-dimethyl-silane (1.07 g, 7.10 mmol) and imidazole (650 mg, 9.55 mmol) were added to a solution of 2-[(4-amino-2-pyridyl)sulfanyl]ethanol (2.3 g, 6.76 mmol) in DCM (30 mL) and the reaction stirred at ambient temperature for 19 h. The mixture was quenched with water (10 mL) and the aqueous layer extracted with DCM (3×10 mL). The combined organic extracts were washed with brine (10 mL), dried (MgSO₄), filtered and concentrated in vacuo. Purification by flash chromatography (40 g SiO₂, 0 to 100% EtOAc in petroleum ether, loaded in DCM) gave 2-[2-[tert-butyl(dimethyl)silyl]oxyethylsulfanyl]pyridin-4-amine (1.4880 g, 77%) as a yellow oil. ¹H NMR (500 MHz, DMSO-d₆) δ 7.79 (d, J=5.6 Hz, 1H), 6.30 (dd, J=2.0, 0.5 Hz, 1H), 6.20 (dd, J=5.7, 2.1 Hz, 1H), 5.93 (s, 2H), 3.69 (t, J=6.9 Hz, 2H), 3.11 (t, J=6.9 Hz, 2H), 0.82 (s, 9H), 0.00 (s, 6H) ppm; ESI-MS m/z calc. 284.13785, found 285.7 (M+1).

### Intermediate K

### rac-2-[[tert-butyl(dimethyl)silyl]oxy-cyclopropyl-methyl]pyridin-4-amine

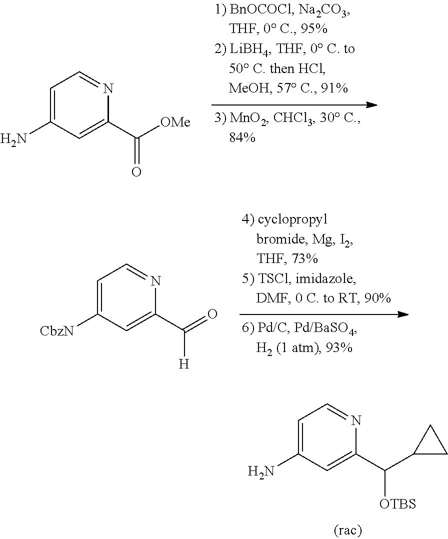

### Step 1:

To a solution of methyl 4-aminopyridine-2-carboxylate (27 g, 94.31 mmol) in THF (800 mL) at 0° C. was added aqueous Na₂CO₃ (210 mL of 2 M, 420 mmol) and then benzyl chloroformate (20.88 g, 17.4 mL, 122.40 mmol) was added via additional funnel. The reaction mixture was stirred for 2 days then the layers were separated and the organic layer concentrated in vacuo. A solution of 85% EtOAc in hexane (500 mL) was added and the resultant solid filtered and dried to give methyl 4-(benzyloxycarbonylamino)pyridine-2-carboxylate as a white solid. The filtrate was concentrated in vacuo. Purification by flash chromatography (100 g SiO₂, 30 to 90% EtOAc in hexane) gave further methyl 4-(benzyloxycarbonylamino)pyridine-2-carboxylate as a white solid. The product batches were combined to give methyl 4-(benzyloxycarbonylamino)pyridine-2-carboxylate (26 g, 95%). ESI-MS m/z calc. 286.0954, found 287.4 (M+1)⁺.

### Step 2:

To a suspension of methyl 4-(benzyloxycarbonylamino)pyridine-2-carboxylate (30 g, 104.79 mmol) in THF (400 mL) at 0° C. was added LiBH₄ (94.3 mL of 2 M, 188.60 mmol) dropwise via addition funnel. The reaction mixture was heated at 50° C. for 1 hour before MeOH (300 mL) was added slowly, followed by addition of 5% HCl (350 mL) until the solution reached pH 2. The reaction mixture was heated at 57° C. overnight, then K₂CO₃ (43 g, 312 mmol) was added and the mixture concentrated in vacuo to remove the MeOH. The residue was diluted in EtOAc (300 mL) and the aqueous layer further extracted with EtOAc (×2). The combined organic extracts were washed with brine, dried (Na₂SO₄) and concentrated in vacuo. The residue was dried in vacuo to give as benzyl N-[2-(hydroxymethyl)-4-pyridyl]carbamate (25 g, 91%) as a white solid. ESI-MS m/z calc. 258.1004, found 259.4 (M+1).

### Step 3:

A suspension of benzyl N-[2-(hydroxymethyl)-4-pyridyl]carbamate (13 g, 50.33 mmol) and manganese dioxide (44 g, 430.20 mmol) in chloroform (200 mL) was stirred at 30° C. under nitrogen for 3 days. The mixture before was filtered through celite, washing through with 1:1 DCM/MeOH (500 mL). The filtrate was concentrated in vacuo, keeping the product under nitrogen to give benzyl N-(2-formyl-4-pyridyl)carbamate (12 g at 90% purity, 84%) as a light yellow foam. ESI-MS m/z calc. 256.0848, found 257.4 (M+1)⁺.

### Step 4:

A flask containing magnesium (57 mg, 2.35 mmol) and iodine (1 mg, 0.20 μL, 0.0039 mmol) was heated using heat gun to initiate reaction, then THF (2.3 mL) was added. Cyclopropyl bromide (311.85 mg, 0.21 mL, 2.55 mmol) was added slowly and the reaction mixture stirred at ambient temperature for 30 min. A solution of benzyl N-(2-formyl-4-pyridyl)carbamate (110 mg, 90% purity, 0.39 mmol) in THF (2.3 mL) was added and the reaction stirred for 30 min and then quenched by the addition of cold saturated aqueous NH₄Cl solution. The aqueous layer was extracted with EtOAc (×2) and the combined organic extracts were washed with brine, dried (Na₂SO₄) and concentrated in vacuo. Purification by flash chromatography (4 g SiO₂, 20 to 80% EtOAc in hexane) gave rac-benzyl N-[2-[cyclopropyl(hydroxy)methyl]-4-pyridyl]carbamate (84 mg, 73%) as a white solid. ESI-MS m/z calc. 298.1317, found 299.6 (M+1)⁺.

### Step 5:

To a solution of rac-benzyl N-[2-[cyclopropyl(hydroxy)methyl]-4-pyridyl]carbamate (8.3 g, 27.82 mmol) and imidazole (5.5 g, 80.79 mmol) in DMF (27 mL) at 0° C. was added tert-butyl-chloro-dimethyl-silane (7.9 g, 52.41 mmol) in 4 portions over 45 min. The reaction was warmed to ambient temperature and stirred for 2 h and then saturated aqueous NH₄Cl and EtOAc were added. The aqueous layer was extracted with EtOAc (×2). The combined organic extracts were washed with brine, dried (Na₂SO₄) and concentrated in vacuo. Purification by flash chromatography (330 g SiO₂, 3 to 35% EtOAc in hexane) gave rac-benzyl N-[2-[[tert-butyl(dimethyl)silyl]oxy-cyclopropyl-methyl]-4-pyridyl]carbamate (10.3 g, 90%) as a white solid. ESI-MS m/z calc. 412.2182, found 413.5 (M+1)⁺.

### Step 6:

rac-Benzyl N-[2-[[tert-butyl(dimethyl)silyl]oxy-cyclopropyl-methyl]-4-pyridyl]carbamate (9.8 g, 23.752 mmol) was dissolved in MeOH (15 mL) and EtOH (55 mL). The reaction vessel was purged with nitrogen before Pd/C (1.9 g, 10% w/w, 1.79 mmol) and Pd/BaSO₄ (0.7 g, 5% w/w, 0.33 mmol) were added. The reaction was stirred under an atmosphere of hydrogen overnight then filtered through celite and the filtrate concentrated in vacuo to give rac-2-[[tert-butyl(dimethyl)silyl]oxy-cyclopropyl-methyl]pyridin-4-amine (6.48 g at 95% purity, 93%) as a white solid. ¹H NMR (500 MHz, DMSO-d₆) δ 7.86 (d, J=5.5 Hz, 1H), 6.56 (d, J=2.3 Hz, 1H), 6.32 (dd, J=5.6, 2.3 Hz, 1H), 5.95 (s, 2H), 4.21 (d, J=5.6 Hz, 1H), 1.12-1.03 (m, 1H), 0.85 (s, 9H), 0.38-0.31 (m, 4H), 0.01 (s, 3H), −0.10 (s, 3H) ppm; ESI-MS m/z calc. 278.1814, found 279.4 (M+1).

### Intermediate L

rel-2-(N,S-dimethylsulfonimidoyl)pyridin-4-amine and rel-2-(N,S-dimethylsulfonimidoyl)pyridin-4-amine

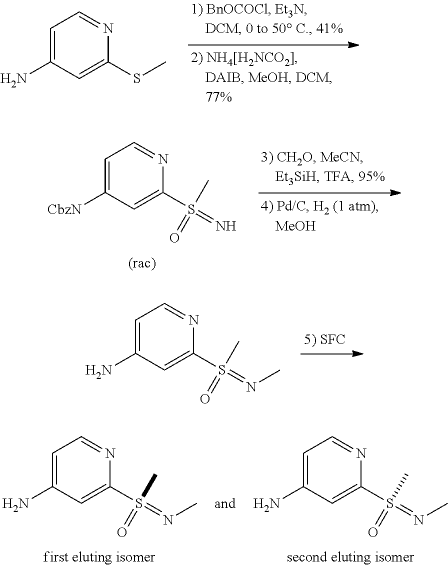

### Step 1:

To a solution of 2-methylsulfanylpyridin-4-amine (1.6 g, 7.99 mmol) in DCM (25 mL) at 0° C. was added Et₃N (2.5 mL, 18 mmol) followed by benzyl chloroformate (1.5 mL, 11 mmol) dropwise over 5 min. The mixture was stirred at 0° C. for 45 min. Et₃N (2.5 mL, 18 mmol) and benzyl chloroformate (1.5 mL, 11 mmol) were added. The reaction was stirred at 0° C. for 1 h, then heated at 50° C. for 1 h. The mixture was again cooled to 0° C. and Et₃N (2.5 mL, 18 mmol) and benzyl chloroformate (1.5 mL, 11 mmol) were added. After 5 min at this temperature the reaction was warmed to ambient temperature and stirred for 16 h before being diluted with DCM and water. The resultant mixture was stirred for 15 min, then passed through a phase separator cartridge and the organic layer was concentrated in vacuo. Purification by flash chromatography (SiO₂, 0 to 20% EtOAc in petroleum ether) gave benzyl N-(2-methylsulfanyl-4-pyridyl)carbamate (901 mg, 41%) as a clear oil which solidified on standing. ¹H NMR (500 MHz, DMSO-d₆) δ 10.22 (s, 1H), 8.24 (dd, J=5.6, 0.6 Hz, 1H), 7.50-7.31 (m, 6H), 7.15 (dd, J=5.7, 2.0 Hz, 1H), 5.18 (s, 2H), 2.47 (s, 3H) ppm; ESI-MS m/z calc. 274.0776, found 275.0 (M+1)⁺; 273.0 (M−1)⁻.

### Step 2:

To a solution of benzyl N-(2-methylsulfanyl-4-pyridyl)carbamate (1.563 g, 5.70 mmol) in MeOH (100 mL) and DCM (100 mL) was added (diacetoxyiodo)benzene (5.965 g, 18.52 mmol) and ammonium carbamate (3.065 g, 39.26 mmol). The reaction was stirred at ambient temperature for 3 days before being concentrated in vacuo. Purification by flash chromatography (40 g SiO₂, 0 to 100% EtOAc in heptane) gave rac-benzyl N-[2-(methylsulfonimidoyl)-4-pyridyl]carbamate (1.337 g, 77%). ¹H NMR (500 MHz, DMSO-d₆) δ 10.63 (s, 1H), 8.51 (d, J=5.5 Hz, 1H), 8.21 (d, J=2.0 Hz, 1H), 7.60 (dd, J=5.5, 2.1 Hz, 1H), 7.48-7.38 (m, 4H), 7.40-7.33 (m, 1H), 5.22 (s, 2H), 4.29 (s, 1H), 3.12 (d, J=1.0 Hz, 3H) ppm; ESI-MS m/z calc. 305.0834, found 306.6 (M+1)⁺; 304.7 (M−1)⁻.

### Step 3:

To a solution of rac-benzyl N-[2-(methylsulfonimidoyl)-4-pyridyl]carbamate (1.33 g, 4.36 mmol) in MeCN (40 mL) was added formaldehyde (600 μL, 21.78 mmol), triethylsilane (2.1 mL, 13.15 mmol) and TFA (1 mL, 12.98 mmol). The reaction mixture was stirred at ambient temperature for 20 h then quenched by addition of saturated aqueous sodium bicarbonate. The aqueous layer was extracted with DCM and the combined organic extracts were dried (MgSO₄) and concentrated in vacuo. Purification by flash chromatography (SiO₂, 0 to 100% 3:1 EtOAc:EtOH in heptane) gave rac-benzyl N-[2-(N,S-dimethylsulfonimidoyl)-4-pyridyl]carbamate (1.318 g, 95%). ¹H NMR (500 MHz, DMSO-d₆) δ 10.64 (s, 1H), 8.56 (d, J=5.5 Hz, 1H), 8.17 (d, J=2.0 Hz, 1H), 7.61 (dd, J=5.5, 2.1 Hz, 1H), 7.56-7.33 (m, 5H), 5.22 (s, 2H), 3.16 (s, 3H), 2.46 (s, 3H) ppm; ESI-MS m/z calc. 319.09906, found 320.6 (M+1)⁺; 318.7 (M−1)⁻.

### Step 4:

MeOH (30 mL) was added to a mixture of rac-benzyl N-[2-(N,S-dimethylsulfonimidoyl)-4-pyridyl]carbamate (1.318 g, 4.13 mmol) and Pd/C (1.22 g of 10% w/w, 1.15 mmol) and the mixture stirred under a hydrogen atmosphere for 20 h. The reaction mixture was filtered through Celite, washing with MeOH, and the filtrate concentrated in vacuo. Purification by chiral SFC using a Chiralcel OJ-H column, 5 m particle size, 25 cm×10 mm from Daicel on a Minigram SFC instrument from Berger Instruments gave:

First eluting isomer: rel-2-(N,S-dimethylsulfonimidoyl)pyridin-4-amine (158 mg, first eluting peak). ¹H NMR (500 MHz, DMSO-d₆) δ 8.09 (d, J=5.5 Hz, 1H), 7.15 (d, J=2.2 Hz, 1H), 6.63-6.55 (m, 3H), 3.08 (s, 3H), 2.45 (s, 3H) ppm.

Second eluting isomer: rel-2-(N,S-dimethylsulfonimidoyl)pyridin-4-amine (136 mg, second eluting peak). ¹H NMR (500 MHz, DMSO-d₆) δ 8.09 (d, J=5.6 Hz, 1H), 7.15 (d, J=2.2 Hz, 1H), 6.61 (dd, J=5.6, 2.3 Hz, 1H), 6.58 (s, 2H), 3.08 (s, 3H), 2.45 (s, 3H) ppm.

### Intermediate M

rel-2-[2,2-dimethyl-1,3-dioxolan-4-yl]-5-fluoro-pyridin-4-amine and rel-2-[2,2-dimethyl-1,3-dioxolan-4-yl]-5-fluoro-pyridin-4-amine

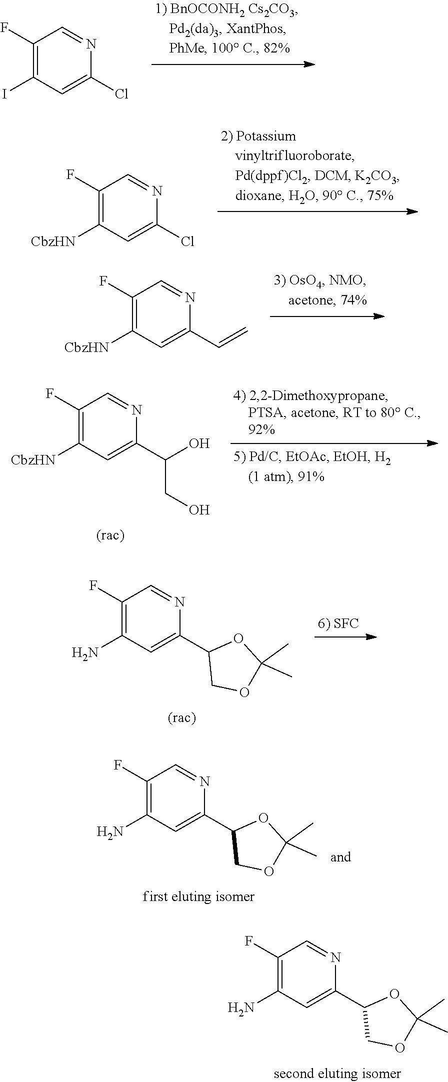

### Step 1:

To a solution of 2-chloro-5-fluoro-4-iodo-pyridine (20 g, 77.69 mmol) in toluene (480 mL) was added benzyl carbamate (12.5 g, 82.692 mmol) and Cs₂CO₃ (50 g, 153.46 mmol). The reaction was purged with argon and Pd₂(dba)₃ (1.43 g, 1.56 mmol) and Xantphos (1.35 g, 2.3331 mmol) were added. The reaction was heated at 100° C. for 5 h then cooled to ambient temperature and filtered through celite, washing with EtOAc (500 mL). The filtrate was washed with water (2×200 mL), brine (200 mL), dried (Na₂SO₄) and concentrated in vacuo. Purification by flash chromatography (SiO₂, 5 to 10% EtOAc in hexane) gave benzyl N-(2-chloro-5-fluoro-4-pyridyl)carbamate (17.8 g, 82%) as a white solid. ¹H NMR (400 MHz, Chloroform-d) δ 8.20 (d, J=5.64 Hz, 1H), 8.13 (d, J=2 Hz, 1H), 7.41-7.36 (m, 5H), 7.12 (brs, 1H), 5.24 (s, 2H) ppm.

### Step 2:

To a solution of benzyl N-(2-chloro-5-fluoro-4-pyridyl)carbamate (10 g, 35.63 mmol) in dioxane (100 mL) and water (10 mL) was added potassium vinyltrifluoroborate (9.75 g, 72.79 mmol). The reaction mixture was degassed with argon before K₂CO₃ (10 g, 72.36 mmol) and Pd(dppf)Cl₂·DCM (3.0 g, 3.67 mmol) were added and the reaction heated at 90° C. for 4 h. The reaction mixture was filtered through celite and diluted with EtOAc (100 mL×2) and water. The combined organic extracts were dried (Na₂SO₄) and concentrated in vacuo. This reaction was repeated 3 further times and the batches combined for purification. Purification by flash chromatography (SiO₂, 10 to 30% EtOAc in hexane) gave N-(5-fluoro-2-vinyl-4-pyridyl)carbamate (32 g, 82%) as a light yellow solid. ¹H NMR (400 MHz, DMSO-d₆) δ 10.17 (s, 1H), 8.43 (d, J=2.7 Hz, 1H), 8.02 (d, J=6.6 Hz, 1H), 7.46-7.33 (m, 5H), 6.76 (dd, J=17.4, 10.7 Hz, 1H), 6.08 (dd, J=17.4, 1.6 Hz, 1H), 5.42 (d, J=10.92 Hz, 1H), 5.22 (s, 2H) ppm.

### Step 3:

To a solution of benzyl N-(5-fluoro-2-vinyl-4-pyridyl)carbamate (9 g, 33.06 mmol) in acetone (90 mL) was added osmium tetroxide (4.2 mL of 4% w/v as solution in water, 0.66 mmol) and NMO (24 mL of 50% w/v, 102.44 mmol). The reaction was stirred at ambient temperature for 3 h then quenched with saturated aqueous Na₂S₂O₃ (50 mL) and the aqueous layer extracted with EtOAc (2×100 mL). The combined organic extracts were washed with brine (50 mL), dried (Na₂SO₄) and concentrated in vacuo. Purification by flash chromatography (SiO₂, 60 to 80% EtOAc in n-hexane) gave rac-benzyl N-[2-(1,2-dihydroxyethyl)-5-fluoro-4-pyridyl]carbamate (7.51 g, 74%) as an off-white solid. ¹H NMR (400 MHz, Chloroform-d) δ 10.08 (s, 1H), 8.36 (s, 1H), 8.09 (d, J=6.72 Hz, 1H), 7.46-7.33 (m, 5H), 5.44 (d, J=4.52 Hz, 1H), 5.21 (s, 2H), 4.66 (t, J=11.6 Hz, 1H), 4.53-4.52 (m, 1H), 3.66-3.61 (m, 1H), 3.47-3.41 (m, 1H) ppm; ESI-MS m/z calc. 306.1016, found 307.3 (M+1)⁺.

### Step 4:

To a solution of rac-benzyl N-[2-(1,2-dihydroxyethyl)-5-fluoro-4-pyridyl]carbamate (12 g, 39.18 mmol) in acetone (120 mL) was added 2,2-dimethoxypropane (11.011 g, 13 mL, 105.73 mmol) and PTSA (390 mg, 2.05 mmol). The reaction mixture was stirred at ambient temperature for 16 h and then heated at 80° C. for 2 h. The reaction mixture was quenched with saturated aqueous NaHCO₃ and the aqueous layer extracted with 5% MeOH in DCM (250 mL×4). The combined organic extracts were dried (Na₂SO₄) and concentrated in vacuo. This reaction was repeated and the two batches combined for purification. Purification by flash chromatography (SiO₂, 10 to 30% EtOAc in hexane) gave rac-N-[2-(2,2-dimethyl-1,3-dioxolan-4-yl)-5-fluoro-4-pyridyl]carbamate (25 g, 92%) as a colourless oil. ¹H NMR (400 MHz, DMSO-d₆) δ 10.19 (s, 1H), 8.40 (d, J=2.8 Hz, 1H), 8.17 (d, J=6.6 Hz, 1H), 7.46-7.36 (m, 5H), 5.22 (s, 2H), 5.06 (t, J=6.5 Hz, 1H), 4.34 (dd, J=8.3, 6.8 Hz, 1H), 3.82 (dd, J=8.3, 6.2 Hz, 1H), 1.44 (s, 3H), 1.40 (s, 3H) ppm.

### Step 5:

A solution of rac-benzyl N-[2-(2,2-dimethyl-1,3-dioxolan-4-yl)-5-fluoro-4-pyridyl]carbamate (9 g, 25.99 mmol) in EtOAc (5 mL) and EtOH (5 mL) was degassed with argon before Pd/C (900 mg, 10% w/w, 0.85 mmol) was added. The reaction was stirred under a balloon of hydrogen at ambient temperature for 16 h. the reaction mixture was filtered through celite and the filtrate concentrated in vacuo. Purification by flash chromatography (SiO₂, 40% EtOAc in n-hexane) gave rac-2-(2,2-dimethyl-1,3-dioxolan-4-yl)-5-fluoro-pyridin-4-amine (5.05 g, 91%) as a white solid. ¹H NMR (400 MHz, Chloroform-d) δ 7.99 (d, J=3.24 Hz, 1H), 6.84 (d, J=7.6 Hz, 1H), 6.24 (s, 2H), 4.90 (t, J=13.4 Hz, 1H), 4.27 (t, J=14.8 Hz, 1H), 3.74 (t, J=15 Hz, 1H), 1.42 (s, 3H), 1.37 (s, 3H) ppm; ESI-MS m/z calc. 212.0961, found 212.9 (M+1)⁺.

### Step 6:

chiral SFC separation. SFC Cellulose: Column: Chiralpak ID, 20×250 mm. Mobile phase: 6 to 15% MeOH (containing 20 mM Ammonia), 94 to 85% CO2. Flow: 100 mL/min

First eluting isomer: rel-2-[2,2-dimethyl-1,3-dioxolan-4-yl]-5-fluoro-pyridin-4-amine. ¹H NMR (500 MHz, DMSO-d₆) δ 8.00 (d, J=3.3 Hz, 1H), 6.85 (d, J=7.7 Hz, 1H), 6.23 (s, 2H), 4.91 (t, J=6.7 Hz, 1H), 4.28 (dd, J=8.1, 6.8 Hz, 1H), 3.75 (dd, J=8.2, 6.8 Hz, 1H), 1.43 (s, 3H), 1.38 (s, 3H) ppm.

Second eluting isomer: rel-2-[2,2-dimethyl-1,3-dioxolan-4-yl]-5-fluoro-pyridin-4-amine. ¹H NMR (500 MHz, DMSO-d₆) δ 8.00 (d, J=3.3 Hz, 1H), 6.85 (d, J=7.6 Hz, 1H), 6.23 (s, 2H), 4.91 (t, J=6.7 Hz, 1H), 4.28 (dd, J=8.2, 6.7 Hz, 1H), 3.75 (dd, J=8.2, 6.8 Hz, 1H), 1.43 (s, 3H), 1.38 (s, 3H) ppm.

### Intermediate N

### rac-6-(2,2-dimethyl-1,3-dioxolan-4-yl)pyridazin-4-amine

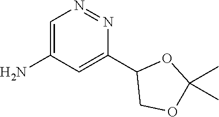

rac-6-(2,2-dimethyl-1,3-dioxolan-4-yl)pyridazin-4-amine was made using the same general method as Intermediate M, except 1:1 MeOH/EtOAc was used as the solvent system for Step 5, and using an alternative to Step 1 as described below:

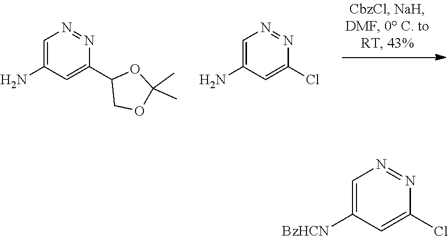

### Alternative Step 1:

To a solution of 6-chloropyridazin-4-amine (3 g, 23.16 mmol) in DMF (20 mL) at 0-5° C. was added NaH (2 g, 60% w/w, 50.01 mmol). The reaction was stirred at 0-5° C. for 30 min before benzyl chloroformate (10 mL of 50% w/v, 29.310 mmol) was added dropwise. The reaction was warmed to ambient temperature and stirred for 3 h then quenched with ice-cold water. The aqueous layer was extracted with 5% MeOH in DCM (2×250 mL) and the combined organic extracts were dried (Na₂SO₄) and concentrated in vacuo. Purification by flash chromatography (SiO₂, 20 to 30% EtOAc in hexane) gave benzyl N-(6-chloropyridazin-4-yl)carbamate (2.6 g, 43%) as a light yellow solid. ¹H NMR (400 MHz, DMSO-d₆) δ 10.86 (s, 1H), 9.12 (d, J=2.3 Hz, 1H), 7.85 (d, J=2.2 Hz, 1H), 7.46-7.35 (m, 5H), 5.23 (s, 2H) ppm.

### Intermediate O

### rac-2-(2,2-dimethyl-1,3-dioxolan-4-yl)-5-methylpyridin-4-amine

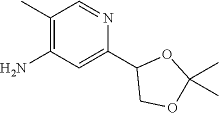

rac-2-(2,2-dimethyl-1,3-dioxolan-4-yl)-5-methylpyridin-4-amine was made using the same general method as Intermediate M, except EtOAc was used as the solvent for Step 5, and the following methylation step was used between Steps 4 and 5:

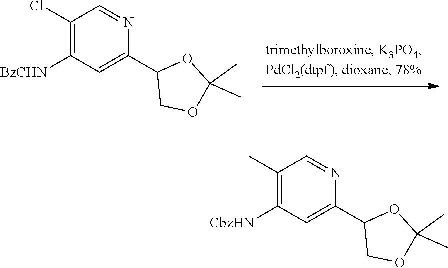

To a solution of rac-benzyl N-[5-chloro-2-(2,2-dimethyl-1,3-dioxolan-4-yl)-4-pyridyl]carbamate (1 g, 2.76 mmol) in 1,4 dioxane (10 mL) was added trimethylboroxine (350 mg, 2.79 mmol) followed by K₃PO₄ (1.5 g, 7.07 mmol). The flask was purged with argon before PdCl₂(dtbpf) (100 mg, 0.15 mmol) was added. The reaction was heated at 100° C. for 5 h, then cooled to ambient temperature and filtered through Celite, washing with EtOAc (2×50 mL). The filtrate was washed with water (50 mL) and brine (50 mL) and then dried (Na₂SO₄) and concentrated in vacuo. Purification by flash chromatography (SiO₂, 5% EtOAc in n-hexane) gave rac-benzyl N-[2-(2,2-dimethyl-1,3-dioxolan-4-yl)-5-methyl-4-pyridyl]carbamate (0.74 g, 78%) as an off-white gum. ¹H NMR (400 MHz, DMSO-d₆) δ 9.29 (s, 1H), 8.22 (s, 1H), 7.91 (s, 1H), 7.46-7.33 (m, 5H), 5.20 (s, 2H), 5.04 (t, J=13.1 Hz, 1H), 4.33 (t, J=14.9 Hz, 1H), 3.81 (q, J=6.5 Hz, 1H), 2.19 (s, 3H), 1.43 (s, 3H), 1.39 (s, 3H) ppm; ESI-MS m/z calc. 342.158, found 342.8 (M+1)⁺.

### Intermediate P

rel-2-(2,2-dimethyl-1,3-dioxolan-4-yl)pyridin-4-amine and

rel-2-(2,2-dimethyl-1,3-dioxolan-4-yl)pyridin-4-amine

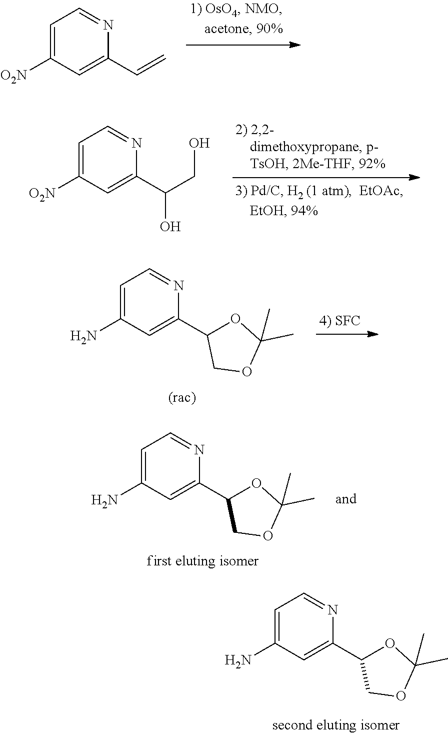

### Step 1:

To a solution of 4-nitro-2-vinyl-pyridine (18 g, 119.89 mmol) in acetone (180 mL) stirring at ambient temperature under a nitrogen atmosphere was added OsO₄ (15.5 mL of 4% w/v, 2.44 mmol) and NMO (85 mL of 50% w/v, 362.80 mmol). The reaction mixture was stirred for 4 h before being quenched by addition of saturated aqueous Na₂S₂O₃ (300 mL) and extracted with EtOAc (2×700 mL). The combined organic extracts were washed with brine (200 mL), dried (MgSO₄) and concentrated in vacuo. Purification by flash chromatography (SiO₂, 0 to 5% MeOH in DCM) gave rac-1-(4-nitro-2-pyridyl)ethane-1,2-diol (20 g, 90%) as a white solid. ¹H NMR (400 MHz, DMSO-d₆) δ 8.88 (d, J=5.5 Hz, 1H), 8.14 (s, 1H), 8.00 (d, J=2.68 Hz, 1H), 5.80 (d, J=5.2 Hz, 1H), 4.76 (dt, J=15.8, 5.5 Hz, 2H), 3.76 (dt, J=10.4, 4.8 Hz, 1H), 3.58 (dt, J=11.8, 6.1 Hz, 1H) ppm; ESI-MS m/z calc. 184.0484, found 185.0 (M+1)⁺.

### Step 2:

To a solution of rac-1-(4-nitro-2-pyridyl)ethane-1,2-diol (35 g, 190.06 mmol) in 2-methyltetrahydrofuran (735 mL) was added 2,2-dimethoxypropane (82 mL, 666.88 mmol) and p-TsOH (4.9 g, 28.455 mmol). The reaction was stirred at ambient temperature for 4 h then diluted with EtOAc (2×700 mL). The organic layer was washed with saturated aqueous NaHCO₃ (400 mL), dried (Na₂SO₄), filtered and concentrated in vacuo. Purification by flash chromatography (SiO₂, 10 to 20% EtOAc in hexane) gave rac-2-(2,2-dimethyl-1,3-dioxolan-4-yl)-4-nitro-pyridine (40 g, 92%) as a white solid. ¹H NMR (400 MHz, DMSO-d₆) δ 8.92 (d, J=5.12 Hz, 1H), 8.07 (s, 2H), 5.30 (t, J=6.4 Hz, 1H), 4.45 (t, J=7.96 Hz, 1H), 3.95-3.92 (m, 1H), 1.48 (s, 3H), 1.44 (s, 3H) ppm; ESI-MS m/z calc. 224.0797, found 224.8 (M+1)⁺.

### Step 3:

A solution of rac-2-(2,2-dimethyl-1,3-dioxolan-4-yl)-4-nitro-pyridine (20 g, 87.60 mmol) in EtOAc (200 mL) and EtOH (200 mL) was degassed with nitrogen gas for 20 min before Pd/C (4 g, 10% w/w, 3.76 mmol) was added. The reaction mixture was stirred under a balloon pressure of hydrogen for 16 h ambient temperature before being filtered through celite, washing with 1:1 EtOH/EtOAc. The filtrate was concentrated in vacuo. Purification by flash chromatography (SiO₂, 0 to 3% MeOH in DCM) gave rac-2-(2,2-dimethyl-1,3-dioxolan-4-yl)pyridin-4-amine (16 g, 94%) as a pale yellow solid. ¹H NMR (400 MHz, DMSO-d₆) δ 7.88 (d, J=5.52 Hz, 1H), 6.62 (s, 1H), 6.35 (d, J=3.64 Hz, 1H), 6.04 (s, 2H), 4.88 (t, J=6.68 Hz, 1H), 4.27 (t, J=7.2 Hz, 1H), 3.73 (t, J=7.2 Hz, 1H), 1.41 (s, 3H), 1.37 (s, 3H) ppm; ESI-MS m/z calc. 194.1055, found 195.2 (M+1)⁺.

### Step 4:

The enantiomers of rac-2-(2,2-dimethyl-1,3-dioxolan-4-yl)pyridin-4-amine were separated by chiral SFC separation. SFC Cellulose: Column: Chiralpak ID, 20×250 mm. Mobile phase: 22% MeOH (containing 20 mM Ammonia), 78% CO2. Flow: 100 mL/min

First eluting isomer (retention time=1.33 minutes): rel-2-[2,2-dimethyl-1,3-dioxolan-4-yl]pyridin-4-amine (1.17 g, 91%). ¹H NMR (500 MHz, DMSO-d₆) δ 7.90 (d, J=5.6 Hz, 1H), 6.63 (d, J=2.3 Hz, 1H), 6.36 (dd, J=5.6, 2.3 Hz, 1H), 6.05 (s, 2H), 4.89 (t, J=6.8 Hz, 1H), 4.28 (dd, J=8.1, 6.8 Hz, 1H), 3.74 (dd, J=8.1, 6.9 Hz, 1H), 1.43 (s, 3H), 1.38 (s, 3H) ppm; ESI-MS m/z calc. 194.105, found 195.2 (M+1)⁺; Retention time: 0.42 minutes.

Second eluting isomer (retention time=2.65 minutes): rel-2-[2,2-dimethyl-1,3-dioxolan-4-yl]pyridin-4-amine (1.18 mg, 91%). ¹H NMR (500 MHz, DMSO-d₆) δ 7.90 (d, J=5.5 Hz, 1H), 6.63 (d, J=2.3 Hz, 1H), 6.36 (dd, J=5.6, 2.3 Hz, 1H), 6.04 (s, 2H), 4.89 (t, J=6.8 Hz, 1H), 4.28 (dd, J=8.1, 6.8 Hz, 1H), 3.75 (dd, J=8.1, 6.9 Hz, 1H), 1.43 (s, 3H), 1.39 (s, 3H) ppm; ESI-MS m/z calc. 194.10553, found 195.6 (M+1)⁺; Retention time: 0.41 minutes.

### Intermediate O

### rac-2-(2,2,4-trimethyl-1,3-dioxolan-4-yl)pyridin-4-amine

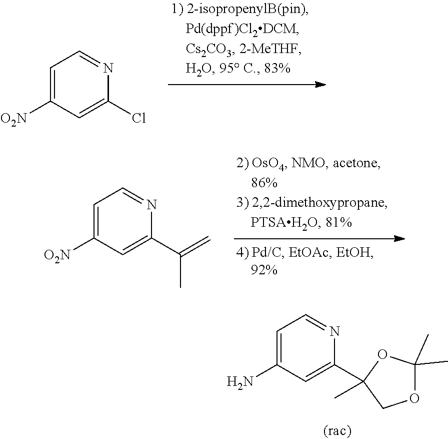

### Step 1:

A solution of 2-chloro-4-nitro-pyridine (40 g, 252.30 mmol), 2-isopropenyl-4,4,5,5-tetramethyl-1,3,2-dioxaborolane (55.160 g, 61.7 mL, 328.25 mmol) and Cs₂CO₃ (164.4 g, 504.58 mmol) in 2-MeTHF (800 mL) and water (40 mL) was degassed with nitrogen gas for 5 min before Pd(dppf)Cl₂·DCM (10.3 g, 12.613 mmol) was added. The reaction was heated at 95° C. for 16 h and then filtered through Celite. The filtrate was diluted with water (500 mL) and extracted with EtOAc (2×1 L). The combined organic extracts were dried (MgSO₄), filtered and concentrated in vacuo. Purification by flash chromatography (SiO₂, 10 to 20% EtOAc in hexane) gave 2-isopropenyl-4-nitro-pyridine (38 g, 83%) as a brown solid. ¹H NMR (400 MHz, DMSO-d₆) δ 8.92 (d, J=5.2 Hz, 1H), 8.23 (s, 1H), 8.00 (t, J=1.64 Hz, 1H), 6.13 (s, 1H), 5.51 (s, 1H), 2.20 (s, 3H) ppm.

### Step 2:

To a solution of 2-isopropenyl-4-nitro-pyridine (40 g, 243.66 mmol) in acetone (400 mL), was added OsO₄ (31 mL of 4% w/v, 4.88 mmol) and NMO (172 mL of 50% w/v, 734.13 mmol). The reaction was stirred at ambient temperature for 2 h before being diluted with water (500 mL) and extracted with EtOAc (3×500 mL). The combined organic layers were dried (MgSO₄) and concentrated in vacuo. Purification by flash chromatography (SiO₂, 0 to 5% MeO in DCM) gave rac-2-(4-nitro-2-pyridyl)propane-1,2-diol (43 g, 86%). ¹H NMR (400 MHz, DMSO-d₆) δ 8.84 (d, J=5.4 Hz, 1H), 8.29 (s, 1H), 7.94 (dd, J=5.4, 2.3 Hz, 1H), 5.50 (s, 1H), 4.71 (t, J=5.8 Hz, 1H), 3.65-3.55 (m, 2H), 1.41 (s, 3H) ppm; ESI-MS m/z calc. 198.0641, found 199.5 (M+1)⁺.

### Step 3:

To a solution of rac-2-(4-nitro-2-pyridyl)propane-1,2-diol (43 g, 216.98 mmol) in 2-methyltetrahydrofuran (900 mL) was added 2,2-dimethoxypropane (72.842 g, 86 mL, 699.41 mmol) and p-TSA monohydrate (6.2 g, 32.59 mmol). The reaction mixture was stirred at ambient temperature for 2 h then diluted with EtOAc (2 L) and washed with saturated aqueous NaHCO₃ (1 L) and brine (1 L). The organic layer was dried (Na₂SO₄) and concentrated in vacuo. Purification by flash chromatography (0 to 20% EtOAc in n-hexane) gave rac-4-nitro-2-(2,2,4-trimethyl-1,3-dioxolan-4-yl)pyridine (43 g, 81%) as a white solid. ¹H NMR (400 MHz, Chloroform-d) δ 8.93 (d, J=5.32 Hz, 1H), 8.18 (d, J=1.96 Hz, 1H), 8.05-8.03 (m, 1H), 4.30 (d, J=8.76 Hz, 1H), 4.11 (d, J=8.68 Hz, 1H), 1.56 (s, 3H), 1.48 (s, 3H), 1.36 (s, 3H) ppm.

### Step 4:

A solution of rac-4-nitro-2-(2,2,4-trimethyl-1,3-dioxolan-4-yl)pyridine (30 g, 125.92 mmol) in EtOAc (600 mL) and EtOH (600 mL) was degassed with argon gas for 20 min before Pd/C (5.2 g, 10% w/w, 4.8863 mmol) was added. The mixture was further degassed for 10 min then stirred under an atmosphere of hydrogen for 5 h. The mixture was filtered through Celite, washing with 1:1 EtOH/EtOAc (400 mL) and the filtrate concentrated in vacuo. Purification by flash chromatography (ammonia treated SiO₂, 2 to 5% MeOH in DCM) gave rac-2-(2,2,4-trimethyl-1,3-dioxolan-4-yl)pyridin-4-amine (24.2 g, 92%) as an off-white solid. ¹H NMR (400 MHz, DMSO-d₆) δ 7.89 (d, J=5.5 Hz, 1H), 6.74 (d, J=2.3 Hz, 1H), 6.31 (dd, J=5.5, 2.3 Hz, 1H), 6.00 (s, 2H), 4.16 (d, J=8.4 Hz, 1H), 3.92 (d, J=8.4 Hz, 1H), 1.42 (d, J=3.5 Hz, 6H), 1.29 (s, 3H) ppm; ESI-MS m/z calc. 208.1212, found 209.0 (M+1).

### Intermediate R

### rac-2-[1-[tert-butyl(dimethyl)silyl]oxy-2-methoxy-ethyl]pyridin-4-amine

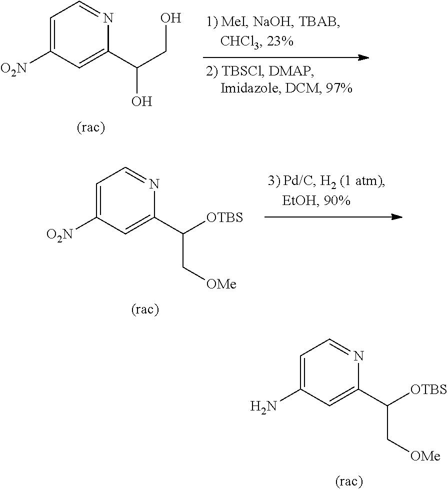 [SMILES: O=[N+]([O-])c1ccnc(C(O)CO)c1 ; COCC(O[Si](C)(C)C(C)(C)C)c1cc([N+](=O)[O-])ccn1 ; COCC(O[Si](C)(C)C(C)(C)C)c1cc(N)ccn1]

### Step 1:

To a solution of rac-1-(4-nitro-2-pyridyl)ethane-1,2-diol (2 g, 10.86 mmol) in chloroform (5 mL) was added NaOH (22 mL of 1 M, 22.00 mmol), methyl iodide (2.9640 g, 1.3 mL, 20.88 mmol) and TBAB (350 mg, 1.09 mmol). The reaction was stirred overnight at ambient temperature. The layers were separated and the aqueous layer extracted with DCM, and the combined organic layers were dried (Na₂SO₄) and concentrated in vacuo. Purification by flash chromatography (SiO₂, 50% EtOAc in hexane) gave rac-2-methoxy-1-(4-nitro-2-pyridyl)ethanol (490 mg, 23%) as a yellow solid.

### Step 2:

To a solution of rac-2-methoxy-1-(4-nitro-2-pyridyl)ethanol (150 mg, 0.76 mmol) in DCM (5 mL) was added imidazole (155 mg, 2.28 mmol), DMAP (9.2 mg, 0.075 mmol) and TBSCl (228 mg, 1.51 mmol). The reaction was stirred overnight at ambient temperature then diluted in water and DCM. The layers were separated and the aqueous layer extracted with DCM, and the combined organic layers were washed with brine, dried (Na₂SO₄) and concentrated in vacuo to give rac-tert-butyl-[2-methoxy-1-(4-nitro-2-pyridyl)ethoxy]-dimethyl-silane (230 mg, 97%) as a light yellow oil. ESI-MS m/z calc. 312.1505, found 313.0 (M+1)⁺.

### Step 3:

rac-Tert-butyl-[2-methoxy-1-(4-nitro-2-pyridyl)ethoxy]-dimethyl-silane (3.2 g, 10.24 mmol) was dissolved in EtOH (100 mL) and the solution degassed for five min. Pd/C (1.6 g, 10% w/w, 1.32 mmol) was added and the reaction stirred under a hydrogen atmosphere for 4 h. The reaction mixture was filtered through Celite and concentrated in vacuo. Purification by flash chromatography (ammonia treated SiO₂, 50 to 70% EtOAc in hexane) gave rac-2-[1-[tert-butyl(dimethyl)silyl]oxy-2-methoxy-ethyl]pyridin-4-amine (2.6 g, 90%) as a white solid. ¹H NMR (400 MHz, Chloroform-d) δ 8.12 (d, J=5.5 Hz, 1H), 6.78 (d, J=2.4 Hz, 1H), 6.38 (dd, J=5.5, 2.4 Hz, 1H), 4.84 (dd, J=6.9, 3.2 Hz, 1H), 4.11 (s, 2H), 3.63 (dd, J=10.3, 3.2 Hz, 1H), 3.50 (dd, J=10.3, 6.8 Hz, 1H), 3.34 (s, 3H), 0.90 (s, 9H), 0.09 (s, 3H), 0.03 (s, 3H) ppm; ESI-MS m/z calc. 282.1764, found 283.0 (M+1)⁺.

### Intermediate S

### rac-2-[2-[tert-butyl(dimethyl)silyl]oxy-1-methoxy-ethyl]pyridin-4-amine

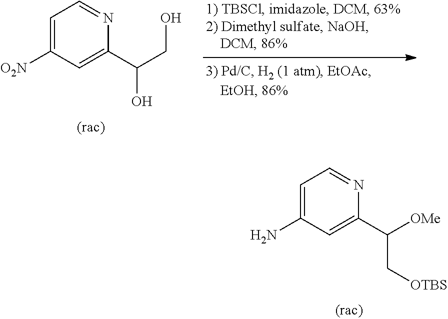 [SMILES: O=[N+]([O-])c1ccnc(C(O)CO)c1 ; COC(CO[Si](C)(C)C(C)(C)C)c1cc(N)ccn1]

### Step 1:

To a stirred solution of rac-1-(4-nitro-2-pyridyl)ethane-1,2-diol (10 g, 54.30 mmol) in DCM (250 mL) was added imidazole (9.8 g, 143.95 mmol) and tert-butyldimethylsilyl chloride (13 g, 86.25 mmol). The reaction mixture was stirred at ambient temperature for 3 h then diluted with water and the aqueous layer extracted with DCM (2×300 mL). The combined organic layers were washed with brine (100 mL), dried (MgSO₄) and concentrated in vacuo. Purification by flash chromatography (SiO₂, 5 to 15% EtOAc in hexane) gave rac-2-[tert-butyl(dimethyl)silyl]oxy-1-(4-nitro-2-pyridyl)ethanol (10.5 g, 63%) as a yellow liquid. ¹H NMR (400 MHz, DMSO-d₆) δ 8.89 (d, J=5.4 Hz, 1H), 8.14 (d, J=2.3 Hz, 1H), 8.01 (dd, J=5.4, 2.3 Hz, 1H), 5.81 (d, J=5.1 Hz, 1H), 4.80 (q, J=4.6 Hz, 1H), 3.92 (dd, J=10.5, 3.9 Hz, 1H), 3.80 (dd, J=10.5, 5.1 Hz, 1H), 0.75 (s, 9H), −0.08 (d, J=17.7 Hz, 6H) ppm; ESI-MS m/z calc. 298.1349, found 299.0 (M+1)⁺.

### Step 2:

To a solution of rac-2-[tert-butyl(dimethyl)silyl]oxy-1-(4-nitro-2-pyridyl)ethanol (10.5 g, 35.19 mmol) in DCM (105 mL) was added NaOH (71 mL of 1 M, 71.00 mmol) and TBAB (23 g, 71.35 mmol). The reaction mixture was stirred at ambient temperature for 15 min. Dimethyl sulphate (9.975 g, 7.5 mL, 79.08 mmol) was reaction mixture was stirred at ambient temperature for 16 h. The reaction mixture was diluted with water (200 mL), extracted with DCM (2×500 mL) and the combined organic extracts were dried (MgSO₄), filtered and concentrated in vacuo. Purification by flash chromatography (SiO₂, 5 to 10% EtOAc in hexane) gave rac-tert-butyl-[2-methoxy-2-(4-nitro-2-pyridyl)ethoxy]-dimethyl-silane (10 g, 86%) as a yellow liquid. ¹H NMR (400 MHz, DMSO-d₆) δ 8.94 (d, J=5.4 Hz, 1H), 8.06 (d, J=5.16 Hz, 1H), 8.02 (s, 1H), 4.53 (t, J=4.3 Hz, 1H), 3.99-3.95 (m, 1H), 3.84-3.80 (m, 1H), 3.34 (s, 3H), 0.75 (s, 9H), −0.09 (d, J=16.9 Hz, 6H) ppm; ESI-MS m/z calc. 312.1505, found 313.3 (M+1)⁺.

### Step 3:

To a solution of rac-tert-butyl-[2-methoxy-2-(4-nitro-2-pyridyl)ethoxy]-dimethyl-silane (6 g, 19.20 mmol) in EtOH (120 mL) and EtOAc (120 mL) was added Pd/C (3 g, 10% w/w, 2.82 mmol). The mixture was stirred under a hydrogen atmosphere for 16 h then filtered through Celite. The filtrate was evaporated in vacuo. Purification by flash chromatography (amine treated SiO₂, 2 to 5% MeOH in DCM) gave rac-2-[2-[tert-butyl(dimethyl)silyl]oxy-1-methoxy-ethyl]pyridin-4-amine (4.8 g, 86%). ¹H NMR (400 MHz, Chloroform-d) δ 8.17 (d, J=5.6 Hz, 1H), 6.65 (d, J=2.4 Hz, 1H), 6.41 (dd, J=5.6, 2.4 Hz, 1H), 4.24 (dd, J=6.5, 3.6 Hz, 1H), 4.11 (s, 2H), 3.89 (dd, J=10.9, 3.6 Hz, 1H), 3.77 (dd, J=10.9, 6.5 Hz, 1H), 3.38 (s, 3H), 0.84 (s, 9H), −0.01-−0.05 (m, 6H) ppm; ESI-MS m/z calc. 282.1764, found 283.0 (M+1)⁺.

### Intermediate T

### rac-tert-butyl N-[1-(4-amino-2-pyridyl)-2-[tert-butyl(dimethyl)silyl]oxy-ethyl]-N-methyl-carbamate

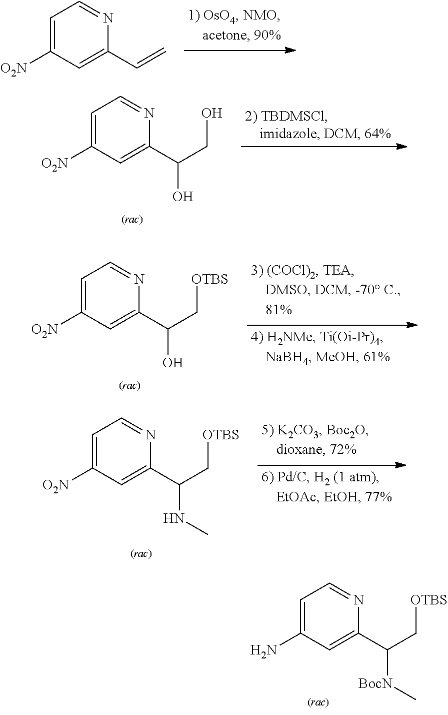

### Step 1:

To a solution of 4-nitro-2-vinyl-pyridine (18 g, 119.89 mmol) in acetone (180 mL) stirring at ambient temperature under a nitrogen atmosphere was added OSO₄ (15.5 mL of 4% w/v, 2.44 mmol) and NMO (85 mL of 50% w/v, 362.80 mmol). The reaction mixture was stirred for 4 h before being quenched by the addition of saturated aqueous Na₂S₂O₃ (300 mL) and extracted with EtOAc (2×700 mL). The combined organic layers were washed with brine (200 mL), dried (MgSO₄) and concentrated in vacuo. Purification by flash chromatography (SiO₂, 0 to 5% MeOH in DCM) gave rac-1-(4-nitro-2-pyridyl)ethane-1,2-diol (20 g, 90%) as a white solid. ¹H NMR (400 MHz, DMSO-d₆) δ 8.88 (d, J=5.5 Hz, 1H), 8.14 (s, 1H), 8.00 (d, J=2.68 Hz, 1H), 5.80 (d, J=5.2 Hz, 1H), 4.76 (dt, J=15.8, 5.5 Hz, 2H), 3.76 (dt, J=10.4, 4.8 Hz, 1H), 3.58 (dt, J=11.8, 6.1 Hz, 1H) ppm; ESI-MS m/z calc. 184.0484, found 185.0 (M+1)⁺.

### Step 2:

To a solution of rac-1-(4-nitro-2-pyridyl)ethane-1,2-diol (10 g, 54.30 mmol) in DCM (250 mL) was added imidazole (9.8 g, 143.95 mmol) and tert-butyldimethylsilyl chloride (13 g, 86.25 mmol). The reaction mixture was stirred at ambient temperature for 3 h, diluted with water and extracted with DCM (2×300 mL). The combined organic extracts were washed with brine (100 mL), dried (MgSO₄) and concentrated in vacuo. Purification by flash chromatography (SiO₂, 5 to 15% EtOAc in hexane) gave rac-2-[tert-butyl(dimethyl)silyl]oxy-1-(4-nitro-2-pyridyl)ethanol (10.5 g, 63%) as a yellow liquid. ¹H NMR (400 MHz, DMSO-d₆) δ 8.89 (d, J=5.4 Hz, 1H), 8.14 (d, J=2.3 Hz, 1H), 8.01 (dd, J=5.4, 2.3 Hz, 1H), 5.81 (d, J=5.1 Hz, 1H), 4.80 (q, J=4.6 Hz, 1H), 3.92 (dd, J=10.5, 3.9 Hz, 1H), 3.80 (dd, J=10.5, 5.1 Hz, 1H), 0.75 (s, 9H), −0.08 (d, J=17.7 Hz, 6H) ppm; ESI-MS m/z calc. 298.1349, found 299.0 (M+1).

### Step 3:

To a solution of oxalyl chloride (9.6030 g, 6.6 mL, 75.658 mmol) in dry DCM (90 mL) at -70° C. under nitrogen was added dropwise a solution of anhydrous DMSO (11 mL, 155 mmol) in DCM (30 mL). The mixture was stirred at −70° C. for 30 min. A solution of rac-2-[tert-butyl(dimethyl)silyl]oxy-1-(4-nitro-2-pyridyl)ethanol (15 g, 50.27 mmol) in DCM (60 mL) was added and the mixture was stirred at −70° C. for 90 min. NEt₃ (22.143 g, 30.5 mL, 218.83 mmol) was added and the reaction mixture was stirred at −70° C. for 90 min. Water (30 mL) was added and the mixture was allowed to warm to ambient temperature. The layers were separated and the aqueous layer was extracted with DCM (3×100 mL). The combined organic extracts were washed with 5% aqueous citric acid (3×200 mL), 5% aqueous Na₂CO₃ (3×200 mL) and brine (3×200 mL). The organic layer was dried (MgSO₄) and concentrated in vacuo to give rac-2-[tert-butyl(dimethyl)silyl]oxy-1-(4-nitro-2-pyridyl)ethanone (12 g, 81%) as a brown liquid. ¹H NMR (400 MHz, DMSO-d₆) δ 9.07 (d, J=5.2 Hz, 1H), 8.40 (dt, J=7.6, 2.2 Hz, 2H), 5.26 (s, 2H), 0.92 (s, 9H), 0.9 (s, 6H) ppm.

### Step 4:

To a solution of rac-2-[tert-butyl(dimethyl)silyl]oxy-1-(4-nitro-2-pyridyl)ethanone (12 g, 40.49 mmol) in MeOH (120 mL) at 0° C. was added methyl amine (41 mL of 2 M in MeOH, 82.00 mmol) and titanium(IV) isopropoxide (57.780 g, 60 mL, 203.30 mmol). The reaction mixture was warmed to ambient temperature and stirred for 30 min before being cooled to 0° C. NaBH₄ (3.1 g, 81.94 mmol) was added and the reaction mixture was stirred for 16 h. The reaction mixture was diluted with H₂O (200 mL), extracted with DCM (2×400 mL) and the combined organic extracts were washed with brine, dried (MgSO₄) and concentrated in vacuo. Purification by flash chromatography (SiO₂, 30 to 40% EtOAc in hexane) gave rac-2-[tert-butyl(dimethyl)silyl]oxy-N-methyl-1-(4-nitro-2-pyridyl)ethanamine (8 g, 61%) as a yellow liquid. ¹H NMR (400 MHz, DMSO-d₆) δ 8.91 (d, J=5.4 Hz, 1H), 8.13 (s, 1H), 8.01-8.00 (m, 1H), 3.88-3.86 (m, 1H), 3.82-3.73 (m, 2H), 2.23 (s, 3H), 0.75 (s, 9H), −0.10 (s, 6H) ppm; ESI-MS m/z calc. 311.1665, found 312.0 (M+1)⁺.

### Step 5:

To a solution of rac-2-[tert-butyl(dimethyl)silyl]oxy-N-methyl-1-(4-nitro-2-pyridyl)ethanamine (8 g, 25.69 mmol) in 1,4-dioxane (80 mL) was added K₂CO₃ (11 g, 79.59 mmol) and Boc₂0 (8.5500 g, 9 mL, 39.18 mmol). The reaction was heated at 90° C. for 3 h, then diluted with H₂O and extracted with DCM (2×300 mL). The combined organic extracts were washed with brine (200 mL), dried (MgSO₄) and concentrated in vacuo. Purification by flash chromatography (SiO₂, 5 to 15% EtOAc in hexane) gave rac-tert-butyl N-[2-[tert-butyl(dimethyl)silyl]oxy-1-(4-nitro-2-pyridyl)ethyl]-N-methyl-carbamate (8 g, 72%) as a yellow liquid. ¹H NMR (400 MHz, DMSO-d₆) δ 8.91 (d, J=5.3 Hz, 1H), 7.98 (d, J=5.4 Hz, 1H), 7.92 (s, 1H), 5.27 (t, J=5.9 Hz, 1H), 4.32-4.13 (m, 2H), 2.87 (s, 3H), 1.39 (s, 9H), 0.87 (s, 9H), 0.08 (s, 6H) ppm; ESI-MS m/z calc. 411.2189, found 412.0 (M+1)⁺.

### Step 6:

To a solution of rac-tert-butyl N-[2-[tert-butyl(dimethyl)silyl]oxy-1-(4-nitro-2-pyridyl)ethyl]-N-methyl-carbamate (7 g, 17.01 mmol) in EtOH (140 mL) and EtOAc (140 mL) was added Pd/C (2.1 g, 10% w/w, 1.9733 mmol). The reaction was stirred under a hydrogen atmosphere for 16 h. The reaction mixture was filtered through celite and the filtrate was concentrated in vacuo. Purification by flash chromatography (SiO₂, 30 to 40% EtOAc in hexane) gave rac-tert-butyl N-[1-(4-amino-2-pyridyl)-2-[tert-butyl(dimethyl)silyl]oxy-ethyl]-N-methyl-carbamate (5 g, 77%) as a white solid. H NMR (400 MHz, DMSO-d₆) δ 7.92 (d, J=5.5 Hz, 1H), 6.42-6.37 (m, 2H), 5.64 (s, 2H), 5.03 (t, J=5.1 Hz, 1H), 4.16-3.97 (m, 2H), 2.76 (s, 3H), 1.41 (s, 9H), 0.88 (s, 9H), 0.06 (s, 6H) ppm; ESI-MS m/z calc. 381.2448, found 382.0 (M+1)⁺.

### Intermediate U—‘Syn Diol’

### rac-2-[(4R,5S)-2,2,5-trimethyl-1,3-dioxolan-4-yl]pyridin-4-amine

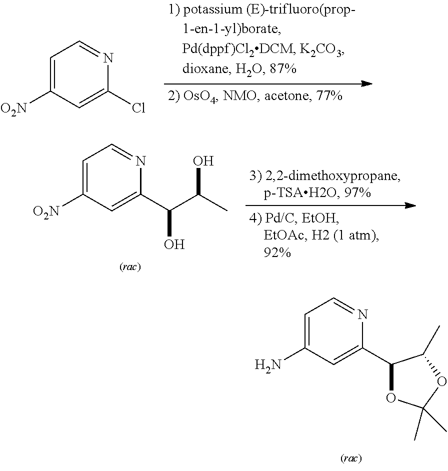

### Step 1:

To a solution of 2-chloro-4-nitro-pyridine (20 g, 126.15 mmol) in 1,4-dioxane (200 mL) and water (20 mL) was added potassium (E)-trifluoro(prop-1-en-1-yl)borate (25 g, 168.95 mmol) followed by K₂CO₃ (35 g, 253.25 mmol). The flask was purged with argon and Pd(dppf)Cl₂·DCM (5.2 g, 6.37 mmol) was added. The reaction was heated at 100° C. for 5 h then cooled to ambient temperature and filtered through celite. The filtrate was washed with water (200 mL), brine (200 mL), dried (Na₂SO₄) and concentrated in vacuo. Purification by flash chromatography (SiO₂, 5% EtOAc in n-hexane) gave 4-nitro-2-[(E)-prop-1-enyl]pyridine (18.52 g, 87%) as a yellow liquid. ¹H NMR (400 MHz, Chloroform-d) δ 8.77 (d, J=5.28 Hz, 1H), 7.90 (d, J=1.44 Hz, 1H), 7.78-7.76 (m, 1H), 6.97-6.88 (m, 1H), 6.61-6.57 (m, 1H), 1.98 (d, J=5.4 Hz, 3H) ppm; ESI-MS m/z calc. 164.0586, found 165.1 (M+1).

### Step 2:

To a solution of 4-nitro-2-[(E)-prop-1-enyl]pyridine (6.5 g, 39.60 mmol) in acetone (70 mL) was added osmium tetroxide (5.5 mL of 4% w/v, 0.87 mmol) and NMO (28 mL of 50% w/v, 119.51 mmol). The reaction was stirred at ambient temperature for 3 h then quenched by addition of aqueous saturated Na₂S₂O₃ solution (100 mL). The aqueous layer was extracted with EtOAc (2×250 mL) and the combined organic extracts were washed with brine (100 mL), dried (Na₂SO₄) and concentrated in vacuo. Purification by flash chromatography (SiO₂, 50 to 80% EtOAc in n-hexane) gave rac-(1R,2S)-1-(4-nitro-2-pyridyl)propane-1,2-diol (6.12 g, 77%) as an off-white solid. ¹H NMR (400 MHz, DMSO-d₆) δ 8.87 (d, J=5.32 Hz, 1H), 8.13 (s, 1H), 7.99-7.98 (m, 1H), 5.65 (d, J=5.96 Hz, 1H), 4.55-4.50 (m, 2H), 3.95-3.91 (m, 1H), 1.11 (d, J=6.36 Hz, 3H) ppm; ESI-MS m/z calc. 198.0641, found 199.0 (M+1)⁺.

### Step 3:

To a solution of rac-(1R,2S)-1-(4-nitro-2-pyridyl)propane-1,2-diol (6.1 g, 30.78 mmol) in 2-methyltetrahydrofuran (120 mL) was added 2,2-dimethoxypropane (6.80 g, 8 mL, 65.29 mmol) and p-TSA·H₂O (590 mg, 3.10 mmol). The reaction mixture was stirred at ambient temperature for 16 h. The reaction mixture was diluted with EtOAc (500 mL), washed with saturated aqueous NaHCO₃ (100 mL) and brine (100 mL), dried (Na₂SO₄) and concentrated in vacuo. Purification by flash chromatography (SiO₂, 10 to 15% EtOAc in n-hexane) gave rac-4-nitro-2-[(4R,5S)-2,2,5-trimethyl-1,3-dioxolan-4-yl]pyridine (7.12 g, 97%) as a light yellow liquid. ¹H NMR (400 MHz, Chloroform-d) δ 8.83 (d, J=5.32 Hz, 1H), 8.24 (d, J=1.88 Hz, 1H), 7.94-7.92 (m, 1H), 4.72 (d, J=8.24 Hz, 1H), 4.02-3.98 (m, 1H), 1.54 (d, J=9.52 Hz, 6H), 1.49 (d, J=6.04 Hz, 3H) ppm.

### Step 4:

To a solution of rac-4-nitro-2-[(4R,5S)-2,2,5-trimethyl-1,3-dioxolan-4-yl]pyridine (7.1 g, 29.80 mmol) in EtOAc (60 mL) and EtOH (60 mL) was added Pd/C (2 g, 10% w/w, 1.8793 mmol). The mixture was degassed with argon then stirred at ambient temperature under a hydrogen atmosphere for 36 h. The reaction mixture was filtered and concentrated in vacuo. Purification by flash chromatography (SiO₂, 0 to 5% MeOH in DCM) gave rac-2-[(4R,5S)-2,2,5-trimethyl-1,3-dioxolan-4-yl]pyridin-4-amine (5.75 g, 92%) as an off-white solid. ¹H NMR (400 MHz, DMSO-d₆) δ 7.91 (d, J=5.56 Hz, 1H), 6.64 (d, J=2 Hz, 1H), 6.38-6.36 (m, 1H), 6.03 (s, 2H), 4.31 (d, J=8.24 Hz, 1H), 3.91-3.84 (m, 1H), 1.40 (s, 6H), 1.30 (d, J=5.96 Hz, 3H) ppm; ESI-MS m/z calc. 208.1212, found 209.4 (M+1).

### Intermediate V—‘Anti Diol’

### rac-2-[(4R,5R)-2,2,5-trimethyl-1,3-dioxolan-4-yl]pyridin-4-amine

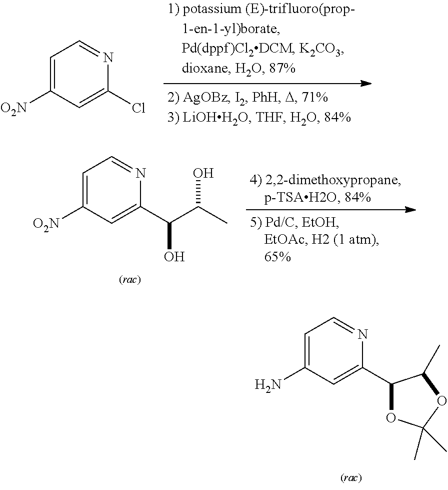

### Step 1:

To a solution of 2-chloro-4-nitro-pyridine (20 g, 126.15 mmol) in 1,4-dioxane (200 mL) and water (20 mL) was added potassium (E)-trifluoro(prop-1-en-1-yl)borate (25 g, 168.95 mmol) followed by K₂CO₃ (35 g, 253.25 mmol). The flask was purged with argon and Pd(dppf)Cl₂.DCM (5.2 g, 6.37 mmol) was added. The reaction was heated at 100° C. for 5 h then cooled to ambient temperature and filtered through Celite. The filtrate was washed with water (200 mL), brine (200 mL), dried (Na₂SO₄) and concentrated in vacuo. Purification by flash chromatography (SiO₂, 5% EtOAc in n-hexane) gave 4-nitro-2-[(E)-prop-1-enyl]pyridine (18.52 g, 87%) as a yellow liquid. ¹H NMR (400 MHz, Chloroform-d) δ 8.77 (d, J=5.28 Hz, 1H), 7.90 (d, J=1.44 Hz, 1H), 7.78-7.76 (m, 1H), 6.97-6.88 (m, 1H), 6.61-6.57 (m, 1H), 1.98 (d, J=5.4 Hz, 3H) ppm; ESI-MS m/z calc. 164.0586, found 165.1 (M+1)⁺.

### Step 2:

To a solution of 4-nitro-2-[(E)-prop-1-enyl]pyridine (12 g, 73.10 mmol) in benzene (200 mL) was added silver(I) benzoate (50 g, 218.36 mmol) and Iodine (30 g, 118.20 mmol) and the mixture was heated at reflux for 36 h. the reaction mixture was diluted with EtOAc (500 mL) and washed with saturated aqueous ammonia solution (200 mL), saturated aqueous NaHCO₃ solution (200 mL) and brine (100 mL). The organic layer was dried (Na₂SO₄) and concentrated in vacuo. Purification by flash chromatography (SiO₂, 5 to 8% EtOAc in n-hexane) gave rac-[(1R,2R)-2-benzoyloxy-1-methyl-2-(4-nitro-2-pyridyl)ethyl]benzoate (22.12 g, 71%) as a yellow liquid. ¹H NMR (400 MHz, Chloroform-d) δ 8.93 (d, J=5.24 Hz, 1H), 8.20 (s, 1H), 8.11 (d, J=7.76 Hz, 2H), 7.98-7.94 (m, 3H), 7.64-7.60 (m, 1H), 7.57-7.47 (m, 3H), 7.43-7.39 (m, 2H), 6.42 (d, J=3.8 Hz, 1H), 5.91-5.87 (m, 1H), 1.46 (d, J=6.48 Hz, 3H) ppm.

### Step 3:

To a solution of rac-[(1R,2R)-2-benzoyloxy-1-methyl-2-(4-nitro-2-pyridyl)ethyl]benzoate (22 g, 54.14 mmol) in THF (155 mL) and water (65 mL) was added LiOH·H₂O (4.5002 g, 107.24 mmol). The reaction was stirred at ambient temperature for 3 h then diluted with EtOAc (500 mL) and washed with water (100 mL) and brine (100 mL). The organic layer was dried (MgSO₄) and concentrated in vacuo. Purification by flash chromatography (SiO₂, 50 to 80% EtOAc in n-hexane) gave rac-(1R,2R)-1-(4-nitro-2-pyridyl)propane-1,2-diol (9.02 g, 84%) as an off-white solid. ¹H NMR (400 MHz, Chloroform-d) δ 8.85 (d, J=5.36 Hz, 1H), 8.17 (s, 1H), 7.98-7.97 (m, 1H), 4.82 (t, J=10.6 Hz, 1H), 4.13 (d, J=4.96 Hz, 1H), 3.63 (d, J=5.8 Hz, 1H), 2.54 (s, 1H), 1.14 (d, J=6.36 Hz, 3H) ppm; ESI-MS m/z calc. 198.0641, found 199.3 (M+1).

### Step 4:

To a solution of rac-(1R,2R)-1-(4-nitro-2-pyridyl)propane-1,2-diol (9 g, 45.41 mmol) in 2-methyltetrahydrofuran (200 mL) was added 2,2-dimethoxypropane (10.20 g, 12 mL, 97.94 mmol) and p-TSA·H₂O (920 mg, 4.84 mmol). The reaction mixture was stirred at ambient temperature for 16 h then diluted with EtOAc (500 mL). The organic layer was washed with saturated aqueous NaHCO₃ solution (100 mL) and brine (100 mL), dried (MgSO₄) and concentrated in vacuo. Purification by flash chromatography (SiO₂, 10 to 15% EtOAc in n-hexane) gave rac-4-nitro-2-[(4R,5R)-2,2,5-trimethyl-1,3-dioxolan-4-yl]pyridine (9.1 g, 84%) as a light yellow liquid. ¹H NMR (400 MHz, Chloroform-d) δ 8.84 (d, J=5.28 Hz, 1H), 8.20-8.19 (m, 1H), 7.92-7.90 (m, 1H), 5.37 (d, J=7.08 Hz, 1H), 4.79-4.72 (m, 1H), 1.70 (s, 3H), 1.50 (s, 3H), 0.81 (d, J=6.4 Hz, 3H) ppm.

### Step 5:

To a solution of rac-4-nitro-2-[(4R,5R)-2,2,5-trimethyl-1,3-dioxolan-4-yl]pyridine (9 g, 37.78 mmol) in EtOAc (80 mL) and EtOH (80 mL) was added Pd/C (4 g, 10% w/w, 3.76 mmol). The mixture was degassed with argon and stirred at ambient temperature under hydrogen in a Parr shaker for 16 h before being filtered through Celite and the filtrate concentrated in vacuo. Purification by flash chromatography (SiO₂, 0 to 5% MeOH in DCM) gave rac-2-[(4R,5R)-2,2,5-trimethyl-1,3-dioxolan-4-yl]pyridin-4-amine (5.16 g, 65%) as a brown liquid. ¹H NMR (400 MHz, DMSO-d₆) δ 7.89 (d, J=5.48 Hz, 1H), 6.59 (d, J=1.56 Hz, 1H), 6.34-6.33 (m, 1H), 6.01 (s, 2H), 4.97 (d, J=6.92 Hz, 1H), 4.56-4.49 (m, 1H), 1.52 (s, 3H), 1.36 (s, 3H), 0.71 (d, J=6.28 Hz, 3H) ppm; ESI-MS m/z calc. 208.1212, found 209.1 (M+1).

### Intermediate W

### rac-2-[3-[tert-butyl(dimethyl)silyl]oxytetrahydrofuran-3-yl]pyridin-4-amine

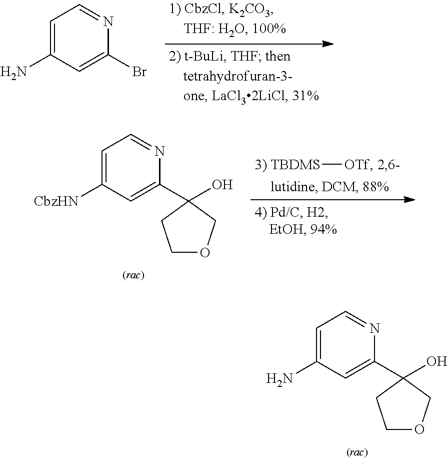

### Step 1:

To a mixture of 2-bromopyridin-4-amine (5 g, 28.90 mmol) in THF (50 mL) and H₂O (30 mL) was added K₂CO₃ (12 g, 86.83 mmol). The reaction was cooled to 0° C. before benzyl chloroformate (8.3 mL, 58.14 mmol) was added dropwise. The reaction mixture was stirred at ambient temperature for 19 h, then partitioned between EtOAc (100 mL) and saturated aqueous NaHCO₃ solution (100 mL). The mixture was stirred for 15 min and the layers were separated. The organic layer was washed with water (20 mL), brine (20 mL), dried (MgSO₄), filtered and concentrated in vacuo. The residue was dissolved in 1:1 MTBE:heptane (100 mL) and stirred for 10 min. The resultant solid was isolated by filtration to give benzyl N-(2-bromo-4-pyridyl)carbamate (9.3391 g, 100%) as an off-white solid. ¹H NMR (500 MHz, Chloroform-d) δ 8.25 (d, J=5.7 Hz, 1H), 7.51 (d, J=1.9 Hz, 1H), 7.44-7.37 (m, 5H), 7.23 (dd, J=5.7, 2.0 Hz, 1H), 6.91 (s, 1H), 5.25 (s, 2H) ppm.

### Step 2:

A mixture of tetrahydrofuran-3-one (2.3 g, 26.72 mmol) and LaCl₃·2LiCl (39.0 mL of 0.6 M in THF, 23.40 mmol) was stirred under nitrogen for 1 hour. In a separate flask, t-BuLi (41.5 mL of 1.7 M in pentanes, 70.55 mmol) was slowly added to a solution of benzyl N-(2-bromo-4-pyridyl)carbamate (7.2 g, 23.44 mmol) in THF (432 mL) at −78° C. and stirred for 15 min. The first solution was added via cannula to the second, and the mixture stirred at −78° C. for 2 h then allowed to warm to ambient temperature over 12 h. The reaction was cooled in an ice-bath and quenched by addition of NaHCO₃ (125 mL) then extracted with EtOAc (3×250 mL). The combined organic extracts were dried (Na₂SO₄), filtered and concentrated in vacuo. Purification by flash chromatography (220 g SiO₂, 0 to 100% EtOAc in hexanes) gave rac-benzyl N-[2-(3-hydroxytetrahydrofuran-3-yl)-4-pyridyl]carbamate (4.52 g, 31%). ESI-MS m/z calc. 314.1267, found 315.4 (M+1)⁺.

### Step 3:

To a solution of rac-benzyl N-[2-(3-hydroxytetrahydrofuran-3-yl)-4-pyridyl]carbamate (4.52 g in ˜50% purity, 7.19 mmol) in DCM (45 mL) was added 2,6-lutidine (4.60 g, 5 mL, 42.93 mmol) and the mixture cooled to 0° C. TBDMSOTf (6.90 g, 6 mL, 26.10 mmol) was added dropwise at 0° C. The reaction mixture was allowed to warm to ambient temperature and stirred for 1 hour. Saturated aqueous NaHCO₃ (60 mL) was added and the mixture was extracted with EtOAc (3×100 mL). The combined organic extracts were dried (Na₂SO₄), filtered and concentrated in vacuo. The residue was diluted in 2:1 DCM/EtOAc, filtered and the filtrate was concentrated in vacuo. Purification by flash chromatography (120 g SiO₂, 0 to 100% EtOAc in hexanes). The obtained oil was further purified by flash chromatography (220 g SiO₂, 0 to 40% EtOAc in hexanes) to give rac-N-[2-[3-[tert-butyl(dimethyl)silyl]oxytetrahydrofuran-3-yl]-4-pyridyl]carbamate (2.74 g, 88%) as a colorless oil. ¹H NMR (250 MHz, Chloroform-d) δ 8.41 (d, J=6 Hz, 1H), 7.54 (d, J=2.0 Hz, 1H), 7.46-7.33 (m, 6H), 6.81 (s, 1H), 5.23 (s, 2H), 4.15-3.93 (m, 4H), 2.69-2.51 (m, 1H), 2.31-2.13 (m, 1H), 0.94 (s, 9H), 0.14-−0.10 (m, 6H) ppm; ESI-MS m/z calc. 428.2131, found 429.3 (M+1);

### Step 4:

To a solution of rac-benzyl N-[2-[3-[tert-butyl(dimethyl)silyl]oxytetrahydrofuran-3-yl]-4-pyridyl]carbamate (2.7 g, 6.30 mmol) in EtOH (50 mL) was added Pd/C (644 mg, 10% w/w, 0.61 mmol). The mixture was cooled to 0° C. and vacuum degassed with hydrogen. The reaction was stirred under a hydrogen atmosphere for 14 h, then flushed with nitrogen. The mixture was diluted with EtOH (150 mL), filtered through Celite, washing with EtOH (2×150 mL) and the filtrate concentrated in vacuo to give rac-2-[3-[tert-butyl(dimethyl)silyl]oxytetrahydrofuran-3-yl]pyridin-4-amine (1.82 g, 94%) as a colorless oil that crystallized on standing. ¹H NMR (500 MHz, DMSO-d₆) δ 7.92 (d, J=5.5 Hz, 1H), 6.68 (d, J=2.2 Hz, 1H), 6.37 (dd, J=5.5, 2.2 Hz, 1H), 6.02 (s, 2H), 4.01-3.94 (m, 1H), 3.94-3.81 (m, 3H), 2.48-2.40 (m, 1H), 2.11-2.03 (m, 1H), 0.86 (s, 9H), 0.01-−0.17 (m, 6H) ppm; ESI-MS m/z calc. 294.1764, found 295.2 (M+1)⁺.

### Intermediate X

### 6-bromo-2-ethyl-3-fluoro-phenol

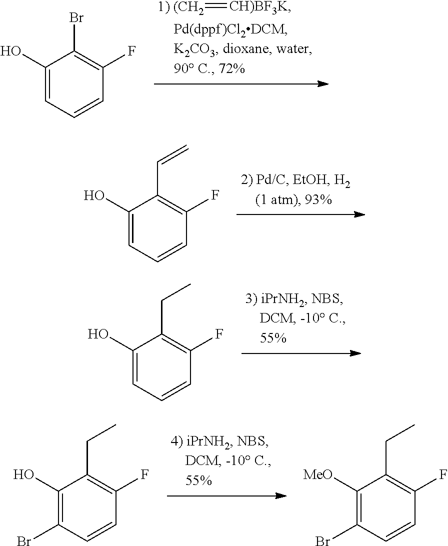

### Step 1:

To a stirred solution of 2-bromo-3-fluoro-phenol (25 g, 130.89 mmol) in a mixture of 1,4-dioxane (250 mL) and water (25 mL) was added potassium vinyl trifluoroborate (52 g, 388.20 mmol) and K₂CO₃ (55 g, 397.96 mmol). The mixture was degassed with nitrogen gas for 15 min before Pd(dppf)Cl₂·DCM (7.5 g, 9.18 mmol) was added and the reaction was heated at 90° C. for 16 h. The reaction mixture was filtered through celite and the filtrate diluted with water (300 mL). The aqueous layer was extracted with EtOAc (2×50 mL) and the combined organic extracts were dried (MgSO₄), filtered and evaporated in vacuo. Purification by flash chromatography (SiO₂, 0 to 1% EtOAc in hexane) gave 3-fluoro-2-vinyl-phenol (14.5 g, 72%) as a colourless liquid. ¹H NMR (400 MHz, Chloroform-d) δ 7.06 (q, J=8.16 Hz, 1H), 6.80-6.73 (m, 1H), 6.66-6.61 (m, 2H), 5.88 (d, J=18.04, 1H), 5.60 (d, J=11.7, 1H), 5.48 (s, 1H) ppm.

### Step 2:

To a solution of 3-fluoro-2-vinyl-phenol (14.5 g, 94.47 mmol) in EtOH (145 mL) that had been degassed with nitrogen was added Pd/C (2.9 g, 10% w/w, 2.39 mmol). The reaction mixture was stirred under a hydrogen atmosphere at ambient temperature for 6 h. The mixture was filtered through celite and the filtrate evaporated in vacuo. Purification by flash chromatography (SiO₂, 0-1% EtOAc in hexane) gave 2-ethyl-3-fluoro-phenol (13 g, 93%) as a colourless liquid. ¹H NMR (400 MHz, Chloroform-d) δ 7.0 (q, J=7.96 Hz, 1H), 6.62 (t, J=8.7 Hz, 1H), 6.54 (d, J=8.08 Hz, 1H), 4.90 (s, 1H), 2.66 (q, J=7.24 Hz, 2H), 1.17 (t, J=7.52 Hz, 3H) ppm.

### Step 3:

To a stirred solution of 2-ethyl-3-fluoro-phenol (13 g, 88.12 mmol) and isopropylamine (4.6920 g, 6.9 mL, 79.38 mmol) in DCM (274 mL) at −10° C. was added NBS (14 g, 78.66 mmol) portion-wise. The reaction was stirred at −10° C. for 15 min before being quenched by addition of 2 M HCl. The aqueous layer was extracted with DCM (500×2 mL) and the combined organic extracts were dried (Na₂SO₄) and concentrated in vacuo. Purification by flash chromatography (SiO₂, 0 to 1% EtOAc in hexane) gave 6-bromo-2-ethyl-3-fluoro-phenol (11 g, 55%) as a colourless liquid. ¹H NMR (400 MHz, Chloroform-d) δ 7.25-7.21 (q, J=5.8 Hz, 1H), 6.55 (t, J=8.72 Hz, 1H), 5.6 (d, J=1.64 Hz, 1H), 2.74 (q, J=7.08 Hz, 2H), 1.16 (t, J=7.4 Hz, 3H) ppm.

### Step 4:

To a mixture of 6-bromo-2-ethyl-3-fluoro-phenol (11 g, 48.21 mmol) and K₂CO₃ (16.5 g, 119.39 mmol) in DMF (110 mL) at 0° C. was added methyl iodide (6 mL, 96.38 mmol) dropwise. The reaction mixture was allowed to warm to ambient temperature and stirred for 12 h. The reaction mixture was diluted with ice-water (250 mL) and extracted with hexane (3×500 mL). The combined organic extracts were dried (MgSO₄), filtered and concentrated in vacuo. Purification by flash chromatography (SiO₂, 0 to 1% EtOAc in hexane) gave 1-bromo-3-ethyl-4-fluoro-2-methoxy-benzene (11 g, 94%) as a colourless liquid. ¹H NMR (400 MHz, Chloroform-d) δ 7.34-7.30 (m, 1H), 6.72 (t, J=8.84 Hz, 1H), 3.84 (s, 3H), 2.73-2.67 (m, 2H), 1.18 (t, J=7.52 Hz, 3H) ppm.

### Intermediate Y

### rac-((4S,5R)-2-(ethoxycarbonyl)-4,5-dimethyl-5-(trifluoromethyl)-4,5-dihydrofuran-3-yl)boronic acid

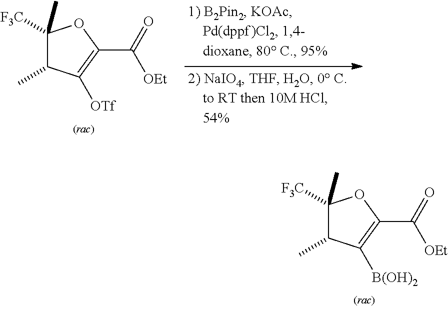 [SMILES: CCOC(=O)C1=C(OS(=O)(=O)C(F)(F)F)[C@H](C)[C@](C)(C(F)(F)F)O1 ; CCOC(=O)C1=C(B(O)O)[C@H](C)[C@](C)(C(F)(F)F)O1]

### Step 1 and 2

To a 3 neck 1 litre flask, flanked with a thermometer and air condenser, was added ethyl rac-(4R,5R)-4,5-dimethyl-5-(trifluoromethyl)-3-(((trifluoromethyl)sulfonyl)oxy)-4,5-dihydrofuran-2-carboxylate (42 g, 108.7 mmol) and 1,4-dioxane (500 mL). The mixture was flushed with nitrogen. KOAc (32 g, 326.1 mmol) was added followed by bis(pinacolato)diboron (32 g, 126.0 mmol). The reaction mixture was evacuated and back filled with nitrogen (×3). Pd(dppf)Cl₂ (4 g, 5.467 mmol) was added and the reaction mixture was heated at 60° C. initially. The reaction was heated at 80° C. under nitrogen for 20 h. The reaction mixture was then cooled to ambient temperature and diluted with ethyl acetate (300 mL) and water (100 mL). The mixture was filtered through a pad of celite, washing several times with ethyl acetate until no more product came off (5×100 ml). The aqueous layer from the filtrates was separated and extracted with ethyl acetate (2×100 mL). The combined organic extracts were dried and filtered using Whatman 1PS hydrophobic phase separator filter paper. The filtrate was concentrated in vacuo. Purification by flash chromatography (Florisil (magnesium silicate) pad, 100% heptane) gave ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (47 g, 95%) as a thick viscous yellow oil. ¹H NMR (500 MHz, Chloroform-d) δ 4.33-4.23 (m, 2H), 3.27-3.18 (m, 1H), 1.55 (d, J=1.1 Hz, 3H), 1.32 (s, 12H), 1.28 (d, J=2.3 Hz, 2H), 1.24 (s, 3H) ppm; ESI-MS m/z calc. 364.1669, found 365.3 (M+1)⁺; Retention time: 1.1 minutes.

NaIO₄ was added (50 g, 233.8 mmol) to a solution of ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (47 g) in a 1:2 mixture of water and THF (150 mL). The reaction mixture was stirred for 1 h. The reaction mixture was cooled down with an ice bath. 1 M HCl (60 mL) was added and reaction mixture was stirred for 60 min. The mixture was diluted with water (50 mL) and ethyl acetate (100 mL). A white solid was filtered and washed with EtOAc. The filtrate was collected and the phases separated. The organic layer was washed with sodium thiosulphate (3×50 ml), brine, dried (Na₂SO₄), filtered and concentrated in vacuo. The cream solid was triturated with cold heptane to give rac-((4S,5R)-2-(ethoxycarbonyl)-4,5-dimethyl-5-(trifluoromethyl)-4,5-dihydrofuran-3-yl)boronic acid (16.657 g, 54%). ¹H NMR (500 MHz, Chloroform-d) δ 6.84 (s, 2H), 4.38 (q, J=7.1 Hz, 2H), 3.18 (q, J=7.3 Hz, 1H), 1.51 (d, J=1.2 Hz, 3H), 1.39 (t, J=7.1 Hz, 3H), 1.32 (dq, J=7.2, 2.4 Hz, 3H) ppm; ESI-MS m/z calc. 282.088, found 281.2 (M−1)⁻; Retention time: 0.75 minutes.

### Intermediate Z

### 1-bromo-4-fluoro-2-methoxy-3-(methoxymethyl)benzene

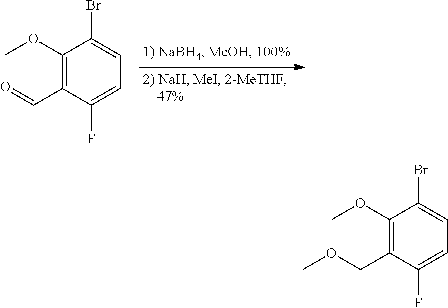

### Step 1:

To a solution of 3-bromo-6-fluoro-2-methoxy-benzaldehyde (2.93 g, 12.57 mmol) in MeOH (50 mL) at 0° C. was added NaBH₄ (449 mg, 11.87 mmol). The reaction mixture was stirred for 10 min and then quenched with 1 M HCl. The aqueous layer was extracted twice with EtOAc and the combined organic extracts were dried (MgSO₄) and concentrated in vacuo to give (3-bromo-6-fluoro-2-methoxy-phenyl)methanol (3.04 g, 103%). ¹H NMR (400 MHz, Methanol-d₄) δ 7.55 (dd, J=8.9, 6.1 Hz, 1H), 6.89 (t, J=8.9 Hz, 1H), 4.69 (d, J=1.9 Hz, 2H), 3.91 (s, 3H) ppm; ESI-MS m/z calc. 233.969, found 218.6 (M-F)⁺; Retention time: 0.65 minutes.

### Step 2:

To a solution of (3-bromo-6-fluoro-2-methoxy-phenyl)methanol (100 mg, 0.4254 mmol) in 2-MeTHF (2 mL) was added NaH (25 mg of 60% w/w, 0.6251 mmol). The reaction was stirred for 15 min followed by the addition of MeI (45 μL, 0.7228 mmol). The reaction mixture was stirred at ambient temperature for 4 h. Further MeI (45 μL, 0.7228 mmol) was added and the reaction stirred at ambient temperature overnight. A saturated aqueous solution of NH₄Cl was added followed by the addition water and EtOAc. The mixture was passed though Whatman phase separator paper and washed with EtOAc. The organic layer was dried (MgSO₄) and concentrated in vacuo. Purification by flash column chromatography (SiO₂, 0-50% EtOAc in heptane) gave 1-bromo-4-fluoro-2-methoxy-3-(methoxymethyl)benzene (50 mg, 47%). ¹H NMR (400 MHz, Chloroform-d) δ 7.49 (dd, J=8.9, 6.1 Hz, 1H), 6.80 (t, J=8.7 Hz, 1H), 4.53 (d, J=2.1 Hz, 2H), 3.91 (s, 3H), 3.41 (d, J=0.6 Hz, 3H) ppm; ESI-MS m/z calc. 247.98482, found 248.9 (M−1)⁻; Retention time: 0.84 minutes.

### Intermediate AA

### 4-Fluoro-2-methoxy-3-methylphenyl)boronic acid

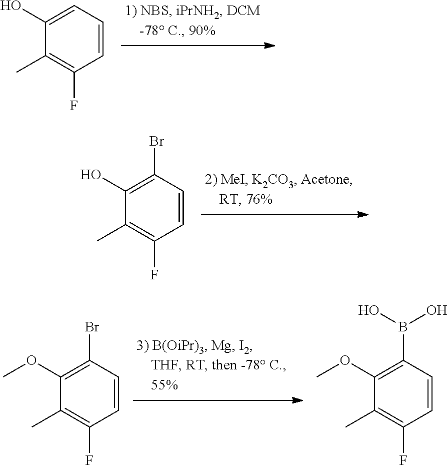

### Step 1:

To a solution of 3-fluoro-2-methyl-phenol (50 g, 396.42 mmol) in DCM (2.5 L) was added isopropylamine (23.460 g, 34.5 mL, 396.89 mmol) and the reaction mixture was cooled to −78° C. NBS (70 g, 393.29 mmol) was added portion wise over 2 h and the mixture was stirred for a further 30 min. The mixture was warmed up to 25° C. 2 N HCl (500 ml) was added and the mixture was stirred for 15 min. The organic layer was separated and concentrated in vacuo, keeping the water bath at 15° C. Hexane (500 ml) was added to the residue and the mixture was stirred for 10 min. The mixture was filtered and the filtrate was concentrated in vacuo, keeping the water bath at 15° C. to give 6-bromo-3-fluoro-2-methyl-phenol (73 g, 90%) as a light brown oil. ¹H NMR (400 MHz, Chloroform-d) δ 7.24-7.21 (m, 1H), 6.55 (t, J=8.8 Hz, 1H), 5.61 (s, 1H), 2.20 (s, 3H) ppm.

### Step 2:

To a stirred solution of 6-bromo-3-fluoro-2-methylphenol (40 g, 195.10 mmol) in acetone (400 mL) at ambient temperature was added potassium carbonate (135 g, 976.80 mmol). The reaction mixture was stirred at 25° C. for 10 min. Methyl iodide (39 g, 17.105 mL, 274.77 mmol) was added dropwise over 10 min and the mixture was stirred at 25° C. for 16 h. The reaction mixture was filtered and the solid residues washed with acetone (50 ml). The filtrate was concentrated at 15° C. in vacuo. Hexane (200 ml) was added and the mixture was stirred for 15 min. The solid was collected and washed with hexane (8 ml). The mother liquors were concentrated in vacuo at 15° C. Purification by distillation (520 mm Hg, 192-196° C.) gave 1-bromo-4-fluoro-2-methoxy-3-methylbenzene (32.4 g, 76%). ¹H NMR (400 MHz, Chloroform-d) δ 7.33-7.30 (m, 1H), 6.72 (t, J=8.7 Hz, 1H), 3.80 (s, 3H), 2.23 (s, 3H) ppm.

### Step 3:

Iodine (50 mg, 0.1970 mmol) was added at 25° C. to a stirred mixture of Mg turnings (5 g, 205.72 mmol) in THF (50 mL). The mixture was stirred until the reaction turned into a clear pale yellow colour. 1-bromo-4-fluoro-2-methoxy-3-methylbenzene (2.5 g, 11.4 mmol) was added dropwise at ambient temperature. When reaction initiation was observed, the remaining solution of 1-bromo-4-fluoro-2-methoxy-3-methylbenzene (22.5 g, 102.71 mmol) in THF (200 mL) was added dropwise. The mixture was stirred for 40 min. The reaction mixture was cooled down to −78° C. and triisopropylborate (64.385 g, 79 mL, 342.34 mmol) was added dropwise. The reaction mixture was warmed to ambient temperature and stirred for 16 h. The reaction mixture was quenched by addition of a 2 N aqueous solution of HCl (25 mL) and stirred for 15 min. The mixture was diluted with water (125 mL) and extracted with EtOAc (2×250 mL). The combined organic extracts were separated, washed with water (250 mL), dried (Na₂SO₄) and concentrated in vacuo. Hexane (25 mL) was added to the residue at 0° C. and the mixture was stirred for 5 min. The resulting solid was filtered, washed with 10 mL of chilled hexane and dried to give (4-fluoro-2-methoxy-3-methylphenyl)boronic acid (11.5 g, 55%). ¹H NMR (400 MHz, DMSO-d₆) δ 7.96 (br s, 2H), 7.32 (t, J=8.0 Hz, 1H), 6.88 (t, J=8.7 Hz, 1H), 3.75 (s, 3H), 2.11 (s, 3H) ppm.

### Intermediate AB

### 2-(3-(Difluoromethyl)-4-fluoro-2-methoxyphenyl)-4,4,5,5-tetramethyl-1,3,2-dioxaborolane

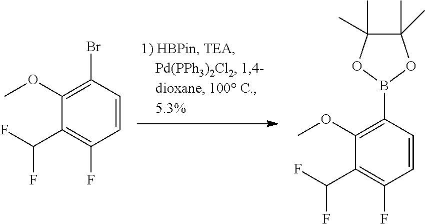 [SMILES: COc1c(Br)ccc(F)c1C(F)F ; COc1c(B2OC(C)(C)C(C)(C)O2)ccc(F)c1C(F)F]

### Step 1:

To a solution of 1-bromo-3-(difluoromethyl)-4-fluoro-2-methoxybenzene (1.60 g, 6.274 mmol) and Pd(PPh₃)₂Cl₂ (200 mg, 0.2849 mmol) in 1,4-dioxane (25 mL) was added 4,4,5,5-tetramethyl-1,3,2-dioxaborolane (1.6 mL, 11.03 mmol) and TEA (2.5 mL, 17.94 mmol). The mixture was degassed by bubbling nitrogen through for 5 min. The reaction mixture was heated at 100° C. in a sealed vial for 3 h and then concentrated in vacuo. Purification by flash chromatography (0 to 25% EtOAc in heptane) gave 2-[3-(difluoromethyl)-4-fluoro-2-methoxy-phenyl]-4,4,5,5-tetramethyl-1,3,2-dioxaborolane (1.15 g, 53%) as a white solid. ¹H NMR (400 MHz, Chloroform-d) δ 7.84 (ddt, J=8.2, 6.9, 1.2 Hz, 1H), 6.99 (td, J=53.9, 1.1 Hz, 1H), 6.91 (dd, J=9.7, 8.5 Hz, 1H), 3.90 (s, 3H), 1.36 (s, 12H) ppm; ESI-MS m/z calc. 302.1301, Retention time: 1.03 minutes.

### Intermediate AC

### 2-(2-ethoxy-3,4-difluorophenyl)-4,4,5,5-tetramethyl-1,3,2-dioxaborolane

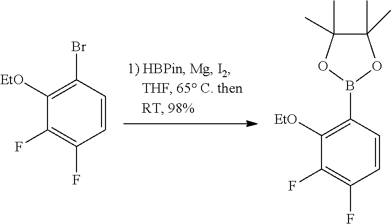 [SMILES: CCOc1c(Br)ccc(F)c1F ; CCOc1c(B2OC(C)(C)C(C)(C)O2)ccc(F)c1F]

### Step 1:

An oven dried 250 ml three necked flask was flanked with an air condenser, an additional funnel and a thermometer. Magnesium (1.8 g, 74.06 mmol) turnings were added. The flask was evacuated three times with vac/N₂ and then left under vacuum for 10 min while the flask was heated to 65° C. Using a nitrogen flushed needle, THF (35 mL) was added to the flask and the mixture was flushed once again with nitrogen. Iodine (5 mg, 0.01970 mmol) was added to the reaction. The mixture was stirred at 65° C. until the reaction turned into a clear pale yellow colour (˜30 min) and then heating was stopped. Pinacolborane (5.5 mL, 37.91 mmol) was added dropwise. A solution of 1-bromo-2-ethoxy-3,4-difluorobenzene (6.8 g, 28.69 mmol) in THF (35 mL) was added dropwise via additional funnel. The reaction mixture was left to cool overnight to ambient temperature. The reaction mixture was added carefully dropwise over 30 min to a stirred solution of 1 M HCl (50 ml) (vigorous effervescence observed) and left to stand for 1 h until all the Mg solids had dissolved. The mixture was diluted with TBME (100 mL). The aqueous layer was separated and extracted twice with TBME. The combined organic extracts were dried (MgSO₄), filtered and concentrated in vacuo to give 2-(2-ethoxy-3,4-difluorophenyl)-4,4,5,5-tetramethyl-1,3,2-dioxaborolane (8 g, 98%). ¹H NMR (400 MHz, Chloroform-d) δ 7.38 (ddd, J=8.6, 6.5, 2.2 Hz, 1H), 6.87 (ddd, J=9.6, 8.5, 6.6 Hz, 1H), 4.14 (qd, J=7.0, 1.0 Hz, 2H), 1.40 (td, J=7.0, 0.6 Hz, 3H), 1.34 (s, 12H) ppm; ESI-MS m/z calc. 284.13953, found 285.4 (M+1)⁺; Retention time: 1.12 minutes.

### Intermediate AD

### 2-(4-(benzyloxy)-3-fluoro-2-methoxyphenyl)acetic acid

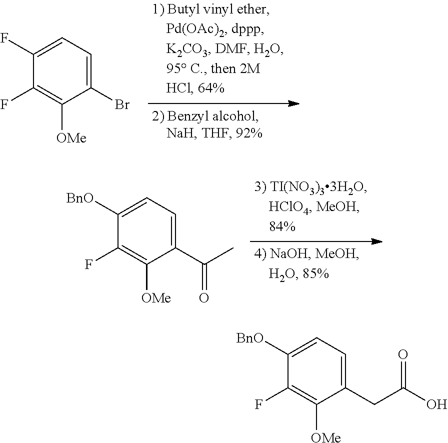 [SMILES: COc1c(Br)ccc(F)c1F ; COc1c(C(C)=O)ccc(OCc2ccccc2)c1F ; COc1c(CC(=O)O)ccc(OCc2ccccc2)c1F]

### Step 1:

A mixture of 1-bromo-3,4-difluoro-2-methoxybenzene (5 g, 22.42 mmol), butyl vinyl ether (9 mL, 66.49 mmol), K₂CO₃ (3.7372 g, 27.04 mmol), dppp (612.81 mg, 1.486 mmol), and Pd(OAc)₂ (151.96 mg, 0.677 mmol) in DMF (50 mL) and H₂O (5 mL) was heated at 95° C. under a nitrogen atmosphere overnight. 2 M HCl (80 mL, 160.0 mmol) was added at ambient temperature and the mixture was stirred for 30 min. The mixture was extracted with EtOAc (2×20 mL). The combined organic extracts were washed with NaHCO₃ (10 mL of saturated aqueous solution), then brine, dried (MgSO₄), filtered and concentrated in vacuo. Purification by flash chromatography (120 g SiO₂, 0 to 5% EtOAc in hexanes) gave 1-(3,4-difluoro-2-methoxyphenyl)ethan-1-one (2.687 g, 64%) as a yellow oil. ¹H NMR (400 MHz, Chloroform-d) δ 7.50 (ddd, J=9.0, 6.1, 2.3 Hz, 1H), 6.92 (td, J=9.0, 6.9 Hz, 1H), 4.08 (d, J=2.7 Hz, 3H), 2.60 (s, 3H) ppm. 1⁹F NMR (376 MHz, Chloroform-d) δ−129.21 (d, J=19.0 Hz), −153.39 (d, J=19.0 Hz) ppm.

### Step 2:

To a stirred suspension of sodium hydride (1.05 g, 60% w/w, 26.253 mmol) in DMF (40 mL) at ambient temperature was added a solution of benzyl alcohol (2.9 g, 26.818 mmol) in DMF (10 mL) and the mixture was stirred for 5 min. 1-(3,4-difluoro-2-methoxyphenyl)ethan-1-one (5 g, 26.859 mmol) was added and the reaction mixture was stirred at ambient temperature for 30 min. HCl (10 mL of 2 N aqueous solution) and brine (100 mL) were added and the mixture was extracted with EtOAc (100 mL then 50 mL). The combined organic extracts were washed with brine (2×20 mL), dried (Na₂SO₄), filtered and concentrated in vacuo. Purification by flash chromatography (SiO₂, 10 to 30% EtOAc in heptane) gave 1-(4-(benzyloxy)-3-fluoro-2-methoxyphenyl)ethan-1-one (5.03 g, 68%) as a yellow solid. ¹H NMR (400 MHz, Chloroform-d) δ 7.51 (dd, J=8.9, 2.1 Hz, 1H), 7.44-7.34 (m, 5H), 6.76 (dd, J=8.9, 7.1 Hz, 1H), 5.17 (s, 2H), 4.03 (d, J=2.3 Hz, 3H), 2.58 (s, 3H) ppm; ESI-MS m/z calc. 274.1005, found 273.02 (M−1)⁻; Retention time: 0.98 minutes.

### Step 3:

A solution of 1-(4-(benzyloxy)-3-fluoro-2-methoxyphenyl)ethan-1-one (14.8 g, 53.958 mmol) in MeOH (50 mL) was added dropwise to a stirred solution of Tl(NO₃)₃.3H₂O (24 g, 54.0 mmol) and perchloric acid (50 mL of 60% w/v in water, 298.63 mmol) in MeOH (200 mL). The reaction mixture was stirred at ambient temperature for 4.5 h. The reaction mixture was filtered, washing through with MeOH (2×50 mL). The filtrate was poured into water (1 L) and extracted with dichloromethane (2×200 mL). The combined organic extracts were washed with water (100 mL) and brine (50 mL), dried (Na₂SO₄), filtered and concentrated in vacuo to give methyl 2-(4-benzyloxy-3-fluoro-2-methoxy-phenyl)acetate (15.25 g, 84%) as a yellow oil. ¹H NMR (400 MHz, Chloroform-d) δ 7.44-7.32 (m, 5H), 6.84 (dd, J=8.7, 1.8 Hz, 1H), 6.67 (t, J=8.2 Hz, 1H), 5.11 (s, 2H), 3.93 (d, J=1.8 Hz, 3H), 3.69 (s, 3H), 3.58 (s, 2H) ppm; ESI-MS m/z calc. 304.1111, found 305.19 (M+1); Retention time: 2.44 minutes.

### Step 4:

Methyl 2-(4-benzyloxy-3-fluoro-2-methoxy-phenyl)acetate (15.2 g, 49.949 mmol) was added to a solution of sodium hydroxide (6 g, 150.01 mmol) in MeOH (30 mL) and water (10 mL). The reaction mixture was stirred at ambient temperature for 14 h and then diluted with 2 N sodium hydroxide solution (200 mL) and washed with dichloromethane (2×30 mL). The aqueous layer was acidified with 6 M hydrochloric acid (100 mL) and extracted with dichloromethane-isopropanol (9:1, 2×150 mL). The combined organic extracts were dried (Na₂SO₄), filtered and concentrated in vacuo to give 2-(4-(benzyloxy)-3-fluoro-2-methoxyphenyl)acetic acid (13.15 g, 85%) as an orange solid. ¹H NMR (400 MHz, Chloroform-d) δ 7.46-7.33 (m, 5H), 6.85 (dd, J=8.7, 1.8 Hz, 1H), 6.69 (t, J=8.0 Hz, 1H), 5.11 (s, 2H), 3.96 (d, J=2.3 Hz, 3H), 3.62 (s, 2H) ppm; OH acid not observed. ESI-MS m/z calc. 290.0954, found 289.0 (M−1)⁻; Retention time: 2.19 minutes.

### Intermediate AE

(R)-4,4,4-trifluoro-3-hydroxy-3-methylbutan-2-one

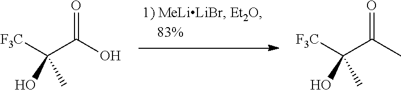

### Step 1:

A jacketed glass reactor, dried and placed under nitrogen atmosphere, was charged with (R)-3,3,3-trifluoro-2-hydroxy-2-methylpropanoic acid (1.0 kg, 6.3261 mol) and diethyl ether (10 L). Methyllithium lithium bromide complex (3.4 L of 1.5 M in Et₂O, 5.1000 mol) was added slowly with evolution of gas and heat formation. The reactor was cooled to maintain a temperature of approximately 16° C. Then methyllithium with lithium bromide (6.1 L of 2.2 M in Et₂O, 13.420 mol) was added slowly. After addition of a total of 2 equivalents, the evolution of gas stopped and the rate of addition was decreased. The mixture was stirred overnight at ambient temperature. The reaction mixture was cooled to 0° C. and transferred to an extraction flask carrying a mixture of water (6 L), ice (2 L) and brine (2 L). The mixture was neutralized by the addition of citric acid (1.6 kg, 960.96 mL, 8.3280 mol) and was stirred for 30 min. The aqueous phase was separated and extracted with diethyl ether (2×2.5 L). The combined organic extracts were concentrated in vacuo to approximately 2 L. The distillate was colored yellow and consisted of 0.8% w/w product. After further distillation only 25 g of product was recovered from the distillate. The distillation residue was further concentrated in a distillation setup with vigreux (30 cm height) at normal pressure. The distillation was continued at reduced pressure (770 mbar) and the pressure was gradually lowered (until 200 mbar) with the collection flask cooled in ice and a cold trap between pump and setup. Mixed fractions were collected until the distillation temperature reached 71° C. The major fraction (590 g) was then collected until the distillation temperature dropped below 70° C. The combined mixed fractions were poured in brine and extracted with diethyl ether (3×75 mL). The combined organic layers were dried (Na₂SO4), filtered and concentrated in a distillation setup at normal pressure. The product was distilled at reduced pressure (200 mbar) to give the product as a colourless oil (198 g). The collected mixed fractions were redistilled to afford more product (44.25 g). All portions of product were combined (857 g), dried by addition of potassium carbonate (52 g) and left standing for 6 h. The water level dropped below detectable level and the mixture was filtered over glass filter to give (R)-4,4,4-trifluoro-3-hydroxy-3-methylbutan-2-one (815 g, 83%) as a colourless oil (815 g). ¹H NMR (300 MHz, Chloroform-d) δ 4.33 (s, 1H), 2.40 (d, J=1.1 Hz, 3H), 1.57 (d, J=1.1 Hz, 3H) ppm. ¹⁹F NMR (282 MHz, Chloroform-d) δ−77.96 ppm.

### Intermediate AF

### rac-2-(2-tert-butoxy-1-fluoro-ethyl)-4-chloro-pyridine

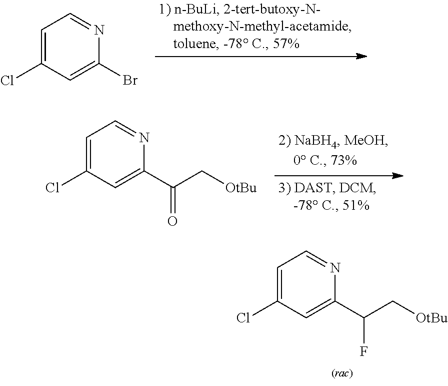

### Step 1:

To a solution of 2-bromo-4-chloro-pyridine (2.5 g, 12.991 mmol) in toluene (50 mL) at -78° C. was added n-BuLi (10 mL of 1.8 M, 18 mmol) and the reaction mixture was stirred at −78° C. for 15 min. A solution of 2-tert-butoxy-N-methoxy-N-methyl-acetamide (2.5 g, 14.267 mmol) in Toluene (12 mL) was added and the reaction mixture was stirred at −78° C. for 45 min. A saturated solution of NH₄Cl was added and the mixture was extracted with EtOAc (2×70 mL). The combined organic extracts were washed with water (40 mL), brine (20 mL), dried (Na₂SO₄) and concentrated in vacuo. Purification by flash chromatography (SiO₂, 0 to 15% EtOAc in heptane) gave 2-tert-butoxy-1-(4-chloro-2-pyridyl)ethanone (1.7 g, 57%) ¹H NMR (400 MHz, DMSO-d₆) δ 8.68 (d, J=5.2 Hz, 1H), 7.93 (d, J=2.0 Hz, 1H), 7.84 (dd, J=4.8 Hz, 1.6 Hz, 1H), 4.93 (s, 2H), 1.19 (s, 9H) ppm.

### Step 2:

To a stirred solution of 2-tert-butoxy-1-(4-chloro-2-pyridyl)ethanone (1.65 g, 9.5061 mmol) in MeOH (30 mL) at 0° C. was added sodium borohydride (800 mg, 21.146 mmol) portion-wise. The reaction mixture was stirred at ambient temperature for 2 h. The reaction mixture was concentrated in vacuo and extracted with ethyl acetate (2×50 mL). The combined organic extracts were concentrated in vacuo. Purification by flash chromatography (SiO₂, 20 to 50% EtOAc in heptane) gave rac-2-tert-butoxy-1-(4-chloro-2-pyridyl)ethanol (1.6 g, 73%) ¹H NMR (400 MHz, DMSO-d₆) δ 8.45 (d, J=5.2 Hz, 1H), 7.54 (s, 1H), 7.42-7.38 (m, 1H), 5.46 (d, J=5.2 Hz, 1H), 4.64-4.59 (m, 1H), 3.63-3.58 (m, 1H), 3.44-3.31 (m, 1H), 1.06 (s, 9H) ppm.

### Step 3:

To a solution of rac-2-tert-butoxy-1-(4-chloro-2-pyridyl)ethanol (1 g, 4.35 mmol) in DCM (20 mL) at −78° C. was added DAST (1.4640 g, 1.2 mL, 9.0825 mmol) drop-wise. The reaction mixture was stirred at −78° C. for 2 h. A saturated sodium bicarbonate solution was added and the mixture was extracted with DCM. The organic extract was dried (Na₂SO₄) and concentrated in vacuo. Purification by flash chromatography (SiO₂, 0 to 50% EtOAc in heptane) gave rac-2-(2-tert-butoxy-1-fluoro-ethyl)-4-chloro-pyridine (580 mg, 51%)¹H NMR (400 MHz, DMSO-d₆) δ 8.55 (d, J=5.2 Hz, 1H), 7.59-7.53 (m, 2H), 5.69-5.54 (m, 1H), 3.89-3.71 (m, 2H), 1.09 (s, 9H) ppm; ESI-MS m/z calc. 231.0826, found 232.0 (M+1)⁺; Retention time: 1.94 minutes.

### Intermediate AG

### 2-[[tert-butyl(dimethyl)silyl]oxymethyl]pyridin-4-amine

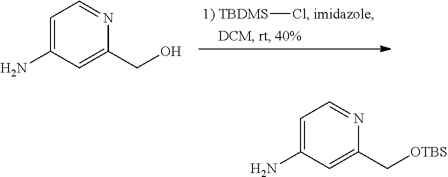

### Step 1:

To a solution of (4-amino-2-pyridyl)methanol (Hydrochloride salt) (1 g, 6.227 mmol) in DCM (15 mL) was added tert-butyl-chloro-dimethyl-silane (495 mg, 3.284 mmol) and imidazole (530 mg, 7.785 mmol). The reaction mixture was stirred at ambient temperature for 17.5 hours. Water (10 mL) was added and the layers separated. The aqueous layer was extracted with EtOAc (3×10 mL). The combined organic extracts were washed with brine (5 mL), dried (MgSO₄), filtered and concentrated in vacuo. Purification by flash chromatography (SiO₂, 0 to 50% EtOAc in heptane) gave 2-[[tert-butyl(dimethyl)silyl]oxymethyl]pyridin-4-amine (602.8 mg, 41%) as a colourless oil. ¹H NMR (500 MHz, Chloroform-d) δ 8.10 (d, J=5.6 Hz, 1H), 6.77 (d, J=1.8 Hz, 1H), 6.41 (dd, J=5.6, 2.4 Hz, 1H), 4.74 (t, J=0.7 Hz, 2H), 4.29 (s, 2H), 0.96 (s, 9H), 0.12 (s, 6H) ppm; ESI-MS m/z calc. 238.150, found 240.8 (M+1)⁺; Retention time: 0.81 minutes.

### Intermediate AH

(6R)-4,6-dimethylpiperazin-2-one

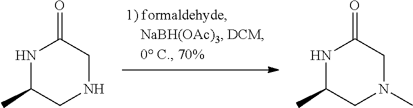

### Step 1:

To a suspension of (6R)-6-methylpiperazin-2-one (3.0 g, 26.28 mmol) in DCM (100 mL) at 0° C. was added formaldehyde (3.2 mL of 37% w/v, 39.43 mmol) and then NaBH(OAc)₃ (11.2 g, 52.84 mmol) in portions. The reaction mixture was stirred at ambient temperature overnight. The reaction mixture was partitioned between DCM and water. The pH of the aqueous layer was adjusted to -pH 9 by the addition of saturated NaHCO₃ solution. The aqueous layer was extracted with EtOAc (×2) and then extracted with 10% IPA/MeOH (˜1 L). The combined organic extracts were dried (Na₂SO₄), filtered and concentrated in vacuo. Purification by flash chromatography (SiO₂, 0 to 50% -[3:1 EtOAc:EtOH] in heptane) gave (6R)-4,6-dimethylpiperazin-2-one (2.51 g, 71%) ¹H NMR (500 MHz, DMSO-d₆) δ 7.74 (s, 1H), 3.46 (dddd, J=12.4, 7.8, 5.3, 3.0 Hz, 1H), 2.95 (dd, J=16.2, 1.4 Hz, 1H), 2.79-2.59 (m, 2H), 2.19 (s, 3H), 1.98 (dd, J=11.5, 8.0 Hz, 1H), 1.06 (d, J=6.3 Hz, 3H) ppm; ESI-MS m/z calc. 128.094, found 129.0 (M+1)⁺; Retention time: 0.2 minutes.

### Intermediate AI

(2R,3S,4S,5R)-3-[3,4-difluoro-2-(trideuteriomethoxy)phenyl]-4,5-dimethyl-5-(trifluoromethyl)tetrahydrofuran-2-carboxylic acid

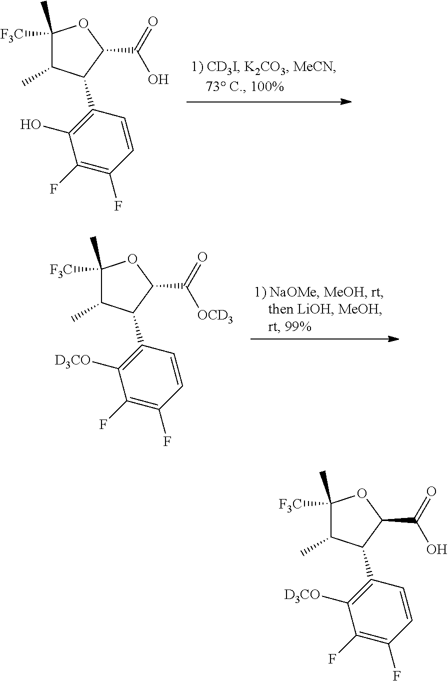

### Step 1:

To a mixture of methyl (2S,3S,4S,5R)-3-(3,4-difluoro-2-hydroxy-phenyl)-4,5-dimethyl-5-(trifluoromethyl)tetrahydrofuran-2-carboxylate (5.0 g, 13.62 mmol) and potassium carbonate (5.6 g, 40.52 mmol) in MeCN (50 mL) was added CD₃I (1.7 mL, 27.31 mmol). The reaction mixture was heated at 73° C. overnight. The reaction mixture was filtered and the filtrate was concentrated in vacuo. The residue was dissolved in TBME and washed with water and brine. The organic phase was dried (MgSO₄), filtered and concentrated in vacuo give trideuteriomethyl (2S,3S,4S,5R)-3-[3,4-difluoro-2-(trideuteriomethoxy)phenyl]-4,5-dimethyl-5-(trifluoromethyl)tetrahydrofuran-2-carboxylate (5.12 g, 100%). ¹H NMR (400 MHz, Chloroform-d) δ 7.18 (ddt, J=9.3, 5.9, 1.9 Hz, 1H), 6.86-6.75 (m, 1H), 4.89 (d, J=6.0 Hz, 1H), 4.25 (dd, J=8.6, 6.0 Hz, 1H), 2.90-2.77 (m, 1H), 1.54 (t, J=1.1 Hz, 3H), 0.88 (dt, J=7.6, 2.0 Hz, 3H) ppm. Retention time: 1.0 minutes.

### Step 2:

Sodium methoxide (0.36 mL of 25% w/v solution in MeOH, 1.666 mmol) was added to a solution of trideuteriomethyl (2S,3S,4S,5R)-3-[3,4-difluoro-2-(trideuteriomethoxy)phenyl]-4,5-dimethyl-5-(trifluoromethyl)tetrahydrofuran-2-carboxylate (5.12 g, 13.68 mmol) in THF (47 mL) at ambient temperature under nitrogen. The reaction mixture was stirred at ambient temperature for 2 h. MeOH (100 mL) and LiOH (10.5 mL of 2 M aqueous solution, 21 mmol) were added and the reaction mixture was stirred for 1 h. The reaction mixture was poured into 2 M HCl then extracted with TBME (2×30 ml). The combined organic extracts were washed with brine, dried (MgSO₄), filtered and concentrated in vacuo to give (2R,3S,4S,5R)-3-[3,4-difluoro-2-(trideuteriomethoxy)phenyl]-4,5-dimethyl-5-(trifluoromethyl)tetrahydrofuran-2-carboxylic acid (4.86 g, 99%). ¹H NMR (400 MHz, Chloroform-d) δ 9.35 (s, 1H), 6.93 (ddd, J=7.9, 5.5, 2.0 Hz, 1H), 6.85 (td, J=9.2, 7.3 Hz, 1H), 4.93 (d, J=10.4 Hz, 1H), 4.12 (dd, J=10.4, 8.0 Hz, 1H), 2.71 (p, J=7.6 Hz, 1H), 1.61 (d, J=1.1 Hz, 3H), 0.76 (dq, J=7.3, 2.3 Hz, 3H) ppm; ESI-MS m/z calc. 357.107, found 356.1 (M−1)⁻; Retention time: 0.58 minutes.

### Intermediate AJ

### 1-(4-bromopyridin-2-yl)-4-methylpiperazin-2-one

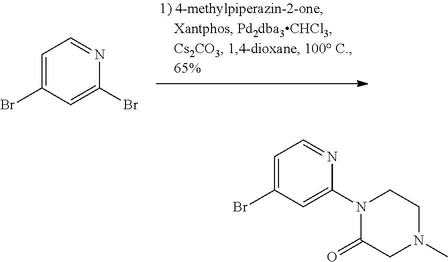

### Step 1:

A mixture of 2,4-dibromopyridine (1.073 g, 4.529 mmol), 4-methylpiperazin-2-one (571 mg, 5.002 mmol), Xantphos (263 mg, 0.4545 mmol) and cesium carbonate (1.71 g, 5.248 mmol) was suspended in 1,4-dioxane (12 mL). The reaction mixture was degassed and purged with nitrogen before addition of Pd₂dba₃·CHCl₃ (237 mg, 0.229 mmol). The vial was sealed and the mixture was heated at 90° C. for 18 h. After cooling down to ambient temperature, the reaction mixture was filtered through a Celite (5 g cartridge), rinsing with EtOAc. The mixture was concentrated in vacuo. The residue was redissolved in MeOH and loaded on to a SCX-2 cartridge (10 g). The column was flushed with MeOH (40 mL) before eluting the desired product using 2 M methanolic ammonia (50 mL). The basic eluent was concentrated in vacuo to give a brown oil. Purification by flash chromatography (24 g SiO₂, 50 to 100% EtOAc in heptane) gave 1-(4-bromopyridin-2-yl)-4-methylpiperazin-2-one (800 mg, 65%). ¹H NMR (400 MHz, DMSO-d₆) δ 8.36 (dd, J=5.3, 0.6 Hz, 1H), 8.19 (dd, J=1.7, 0.5 Hz, 1H), 7.51 (dd, J=5.3, 1.8 Hz, 1H), 3.91-3.83 (m, 2H), 3.21 (s, 2H), 2.77-2.70 (m, 2H), 2.29 (s, 3H) ppm. ESI-MS m/z calc. 269.0164, Retention time: 0.5 minutes.

The following intermediate was made using a method similar to that described in Intermediate AJ except that, tert-butyl 3-oxopiperazine-1-carboxylate was used in place of 4-methylpiperazin-2-one:

| | | | |
|---|---|---|---|
| Intermediate | | | |
| No. | Compound Name | LC/MS | NMR (shifts in ppm) |

| | | |
|---|---|---|
| AK | tert-butyl 4-(4- | ¹H NMR (400 MHz, |
| | bromopyridin-2- | DMSO-d₆) δ 8.37 (dd, J = |
| | yl)-3-oxopiperazine- | 5.4, 0.5 Hz, 1H), 8.18 |
| | 1-carboxylate | (dd, J = 1.8, 0.5 Hz, 1H), |
| | | 7.52 (dd, J = 5.3, 1.8 Hz, |
| | | 1H), 4.16 (s, 2H), 4.04 (t, |
| | | J = 5.5 Hz, 2H), 3.64 (t, |
| | | J = 5.5 Hz, 2H), 1.44 (s, |
| | | 9H) ppm. |

### Intermediate AL

### 4-bromo-N-methyl-N-((2-(trimethylsilyl)ethoxy)methyl)pyridine-2-sulfonamide

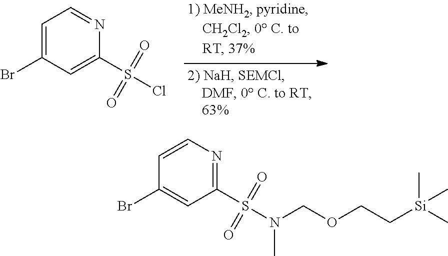 [SMILES: O=S(=O)(Cl)c1cc(Br)ccn1 ; CN(COCC[Si](C)(C)C)S(=O)(=O)c1cc(Br)ccn1]

### Step 1:

MeNH₂ (780 μL, 2 M in THF, 1.560 mmol) was added to a stirred solution of 4-bromopyridine-2-sulfonyl chloride (400 mg, 1.559 mmol) and pyridine (252 μL, 3.116 mmol) in DCM (1.6 mL) at 0° C. After complete addition, the ice bath was removed and the mixture was stirred at ambient temperature for 2 h. The mixture was filtered and the filtrates were diluted with DCM and brine. The aqueous phase was separated and extracted twice with DCM. The combined organic extracts were dried (MgSO₄), filtered and concentrated in vacuo. The residue was triturated twice with heptane. The solid was collected by filtration and dried to give 4-bromo-N-methylpyridine-2-sulfonamide (145 mg, 37%). ¹H NMR (500 MHz, Methanol-d₄) δ 8.55 (dd, J=5.2, 0.6 Hz, 1H), 8.15 (dd, J=1.9, 0.6 Hz, 1H), 7.84 (dd, J=5.2, 1.9 Hz, 1H), 2.68 (s, 3H) ppm; NH amine not observed. ESI-MS m/z calc. 249.94116, found 253.3 (M+1); 249.3 (M−1)⁻; Retention time: 0.51 minutes.

### Step 2:

NaH (60% in mineral oil) (15 mg, 0.375 mmol) was added to a stirred solution of 4-bromo-N-methylpyridine-2-sulfonamide (80 mg, 0.319 mmol) and SEMCl (62 μL, 0.350 mmol) in DMF (1.6 mL) at 0° C. After complete addition, the mixture was stirred at 0° C. for 30 min and for 1 h at ambient temperature. Further amounts of SEMCl (53 mg, 0.318 mmol) and sodium hydride (8 mg, 0.333 mmol) were added and the reaction mixture was stirred for 30 min. The mixture was partitioned between ethyl acetate (15 mL) and water (10 mL). The organic phase was separated and washed with brine (5 mL), dried (MgSO₄), filtered and concentrated in vacuo. Purification by flash chromatography (12 g SiO₂, 0 to 10% ethyl acetate in heptane) gave 4-bromo-N-methyl-N-((2-(trimethylsilyl)ethoxy)methyl)pyridine-2-sulfonamide (76 mg, 63%) as a colourless oil. ¹H NMR (500 MHz, Chloroform-d) δ 8.48 (dd, J=5.2, 0.6 Hz, 1H), 8.13 (dd, J=1.8, 0.6 Hz, 1H), 7.64 (dd, J=5.1, 1.8 Hz, 1H), 4.79 (s, 2H), 3.62-3.51 (m, 2H), 2.97 (s, 3H), 0.94-0.74 (m, 2H), 0.00 (s, 9H) ppm. ESI-MS m/z calc. 380.02255, found 265.3 (M+1)⁺; Retention time: 1.04 minutes.

The following intermediates were made using a method similar to that described in Intermediate AL except that, in Step 1, different pyridinesulfonyl chlorides and amines were respectively used as starting materials in place of 4-bromopyridine-2-sulfonyl chloride and methyl amine. Step 2 was omitted:

| Intermediate | | | |
|---|---|---|---|
| No. | Compound Name | LC/MS | NMR (shifts in ppm) |
| AM | 1-((5-bromopyridin- | ESI-MS m/z calc. | ¹H NMR (500 MHz, |
| | 3-yl)sulfonyl)-4- | 318.99902, found 321.7 | Chloroform-d) δ 8.91- |
| | methylpiperazine | (M + 1)⁺; Retention | 8.84 (m, 2H), 8.16 (t, J = |
| | | time: 0.61 minutes | 2.1 Hz, 1H), 3.15 (s, 4H), |
| | | | 2.55 (s, 4H), 2.32 (s, 3H) |
| | | | ppm. |
| AN | 4-bromo-N,N- | ESI-MS m/z calc. | ¹H NMR (500 MHz, |
| | dimethylpyridine- | 263.95682, found 265.3 | Methanol-d₄) δ 8.56 (dd, |
| | 2-sulfonamide | (M + 1)⁺; Retention | J = 5.2, 0.6 Hz, 1H), 8.13 |
| | | time: 0.65 minutes | (dd, J = 1.9, 0.6 Hz, 1H), |
| | | | 7.87 (dd, J = 5.2, 1.9 Hz, |
| | | | 1H), 2.90 (s, 6H) ppm. |
| AO | 5-bromo-N,N- | ESI-MS m/z calc. | ¹H NMR (500 MHz, |
| | dimethylpyridine- | 263.95682, found 267.3 | Chloroform-d) δ 8.83 (dd, |
| | 3-sulfonamide | (M + 1)⁺; Retention | J = 9.7, 2.0 Hz, 2H), 8.14 |
| | | time: 0.62 minutes | (t, J = 2.1 Hz, 1H), 2.73 |
| | | | (s, 6H) ppm. |

### Intermediate AP

(R)-1-(5-chloropyridazin-3-yl)-4,6-dimethylpiperazin-2-one

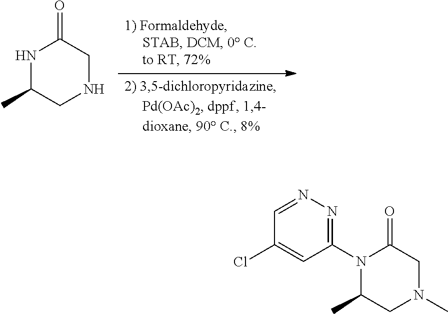

### Step 1:

STAB (6.33 g, 29.867 mmol) was added portionwise to a stirred solution of (R)-6-methylpiperazin-2-one hydrochloride (1 g, 6.640 mmol) and formaldehyde (2.4247 g, 2.2245 mL, 37% w/w in water, 29.879 mmol) in DCM (25 mL) at 0° C. The resulting mixture was stirred overnight at ambient temperature. The reaction mixture was quenched by addition of a saturated aqueous NaHCO₃ solution. The aqueous phase was separated and extracted with DCM. The combined organic extracts were washed with brine, dried (Na₂SO₄), filtered and concentrated in vacuo. Purification by flash chromatography (SiO₂, 0 to 10% methanolic ammonia (7 M solution) in EtOAc) gave (R)-4,6-dimethylpiperazin-2-one (740 mg, 72%) as a pale yellow gum. ¹H NMR (400 MHz, Methanol-d₄) δ 3.70-3.62 (m, 1H), 3.20 (dd, J=16.2, 1.7 Hz, 1H), 2.90-2.84 (m, 2H), 2.35 (s, 3H), 2.15 (dd, J=11.7, 8.6 Hz, 1H), 1.20 (d, J=6.0 Hz, 3H) ppm. ESI-MS m/z calc. 128.095, found 128.97 (M+1)⁺; Retention time: 0.46 minutes.

### Step 2:

3,5-Dichloropyridazine (100 mg, 0.671 mmol), K₃PO₄ (569.9 mg, 2.685 mmol), dppf (55.8 mg, 0.101 mmol) were successively added to a solution of (R)-4,6-dimethylpiperazin-2-one (108.8 mg, 0.705 mmol) in 1,4-dioxane (6 mL). The reaction mixture was degassed for 5 min before adding Pd(OAc)₂ (7.5 mg, 0.033 mmol) to the mixture. The resulting mixture was heated to 90° C. for 3 h. The reaction was cooled to ambient temperature and quenched by addition of water and a saturated aqueous NH₄Cl solution (15 mL). The aqueous phase was separated and extracted with EtOAc (30 mL). The organic extracts were washed with brine, dried (Na₂SO₄), filtered and concentrated in vacuo. Purification by flash chromatography (SiO₂, 33 to 100% ethyl acetate in heptane) gave (R)-1-(5-chloropyridazin-3-yl)-4,6-dimethylpiperazin-2-one (20 mg, 8%) as a brown gum. ¹H NMR (400 MHz, Methanol-d₄) δ 9.17 (t, J=1.3 Hz, 1H), 8.20 (d, J=2.5 Hz, 1H), 4.52-4.27 (m, 1H), 3.46 (d, J=17.1 Hz, 1H), 3.18 (d, J=17.8 Hz, 1H), 2.95-2.73 (m, 2H), 2.42 (s, 3H), 1.26 (d, J=6.1 Hz, 3H) ppm. ESI-MS m/z calc. 240.0778, found 241.12 (M+1)⁺; Retention time: 0.6 minutes.

### Intermediate AQ

### tert-butyl ((4-bromopyridin-2-yl)methyl)(1-methoxy-2-methylpropan-2-yl)carbamate

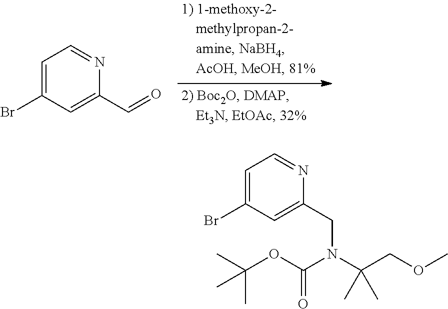 [SMILES: O=Cc1cc(Br)ccn1 ; COCC(C)(C)N(Cc1cc(Br)ccn1)C(=O)OC(C)(C)C]

### Step 1:

1-Methoxy-2-methylpropan-2-amine (378 μL, 2.961 mmol) was added to a stirred solution of 4-bromopicolinaldehyde (500 mg, 2.688 mmol) in MeOH (6.0 mL). One drop of AcOH was then added to the mixture, which was stirred for 180 min at ambient temperature. NaBH₄ (153 mg, 4.044 mmol) was added and the mixture was stirred at ambient temperature overnight. The reaction mixture was diluted with EtOAc (100 mL), washed with a saturated aqueous NaHCO₃ (50 mL) solution and brine (50 mL). The organic phase was passed through a phase separation cartridge, and concentrated in vacuo to give N-((4-bromopyridin-2-yl)methyl)-1-methoxy-2-methylpropan-2-amine (700 mg, 81%), which was used without further purification in the next step. ESI-MS m/z calc. 272.05243, found 275.1 (M+1)⁺; Retention time: 0.7 minutes.

### Step 2:

Boc₂0 (1.1 mL, 4.788 mmol) and DMAP (53 mg, 0.4338 mmol) were successively added to a stirred solution of N-((4-bromopyridin-2-yl)methyl)-1-methoxy-2-methylpropan-2-amine (595 mg, 2.178 mmol) and triethylamine (334 μL, 2.396 mmol) in EtOAc (10 mL). The reaction mixture was stirred at ambient temperature for 135 min. The reaction mixture was washed with water (10 mL), brine (10 mL), dried and filtered through a Whatman 1PS hydrophobic phase separator filter paper. The filtrates were concentrated in vacuo. Purification by flash chromatography (24 g SiO₂, 0 to 30% (3:1 EtOH/EtOAC+2% NH₄OH) in heptane) gave tert-butyl ((4-bromopyridin-2-yl)methyl)(1-methoxy-2-methylpropan-2-yl)carbamate (261 mg, 32%) as a yellow oil. ESI-MS m/z calc. 372.10486, found 375.2 (M+1)⁺; Retention time: 1.0 minutes.

The following intermediate was made using a method similar to that described in Intermediate AQ except that, in Step 1, (S)-pyrrolidin-2-ylmethanol was used in place of 1-methoxy-2-methylpropan-2-amine. Step 2 was omitted:

| | | | |
|---|---|---|---|
| Inter- | | | NMR |
| mediate | | | (shifts |
| No. | Compound Name | LC/MS | in ppm) |

| | | |
|---|---|---|
| AR | (S)-(1-((4-bromopyridin- | ESI-MS m/z calc. |
| | 2-yl)methyl)pyrrolidin- | 270.03677, found 273.1 |
| | 2-yl)methanol | (M + 1)⁺; Retention |
| | | time: 0.59 minutes |

### Intermediate AS

### 2-(6-chloropyrimidin-4-yl)propan-2-ol

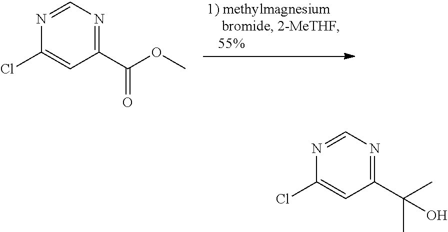

### Step 1:

Methylmagnesium bromide (3.3 mL, 3.2 M in 2-MeTHF, 10.560 mmol) was added to a stirred suspension of methyl 6-chloropyrimidine-4-carboxylate (1 g, 4.9349 mmol) in 2-MeTHF (8 mL) at −40° C. under argon. The reaction mixture was stirred at −40° C. for 30 min. The mixture was quenched by addition of 2 N hydrochloric acid (8 mL) and neutralised to pH 7 by addition of 2 N sodium hydroxide. The biphasic mixture was extracted with ethyl acetate (10 mL). The organic extracts were dried (Na₂SO₄), filtered and concentrated in vacuo. Purification by flash chromatography (12 g SiO₂, 0 to 100% ethyl acetate in heptane) gave 2-(6-chloropyrimidin-4-yl)propan-2-ol (550 mg, 55%) as a colourless oil. ¹H NMR (400 MHz, Chloroform-d) δ 8.91 (s, 1H), 7.53 (d, J=0.9 Hz, 1H), 1.56 (s, 6H) ppm; OH alcohol not observed. ESI-MS m/z calc. 172.0403, found 170.98 (M−1)⁻; Retention time: 0.48 minutes.

The following intermediate was made using a method similar to that described in Intermediate AS except that, 1-(5-bromopyrimidin-2-yl)ethan-1-one was used in place of methyl 6-chloropyrimidine-4-carboxylate as the starting material:

| Intermediate | | | |
|---|---|---|---|
| No. | Compound Name | LC/MS | NMR (shifts in ppm) |
| AT | 2-(5-bromopyrimidin-2- | ESI-MS m/z calc. | ¹H NMR (400 MHz, |
| | yl)propan-2-ol | 215.9898, found 199.05 | Chloroform-d) δ 8.73 (s, |
| | | (M − 17)⁺; Retention | 2H), 4.3-3.8 (brs, 1H), |
| | | time: 0.53 minutes | 1.55 (s, 6H) ppm. |

### Intermediate AU

### rac-5-bromo-2-((tetrahydrofuran-3-yl)methyl)pyridine

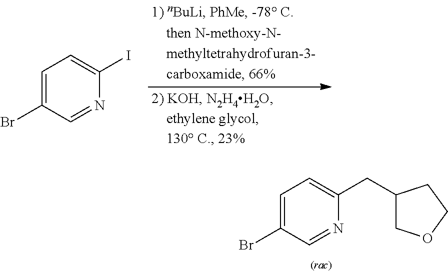 [SMILES: Brc1ccc(I)nc1 ; Brc1ccc(CC2CCOC2)nc1]

### Step 1:

ⁿBuLi (6.4 mL, 2 M solution, 12.800 mmol) was added to a stirred solution of 5-bromo-2-iodopyridine (3 g, 10.567 mmol) in toluene (60 mL) at −78° C. The reaction mixture was stirred for 1 h at at −78° C. A solution of rac-N-methoxy-N-methyltetrahydrofuran-3-carboxamide (2 g, 12.564 mmol) in toluene (10 mL) was added to the reaction mixture at −78° C. and the resulting solution was stirred for 30 min at −78° C. The mixture was quenched by addition of a saturated aqueous NH₄Cl solution (25 mL). The aqueous phase was separated and extracted with ethyl acetate. The combined organic extracts were concentrated in vacuo. Purification by flash chromatography (SiO₂, 3 to 25% ethyl acetate in hexanes) gave rac-(5-bromopyridin-2-yl)(tetrahydrofuran-3-yl)methanone (1.9 g, 66%) as a yellow solid. ESI-MS m/z calc. 254.9895, found 256.0 (M+1)⁺; Retention time: 1.81 minutes.

### Step 2:

KOH (5.5 g, 98.029 mmol) and hydrazine monohydrate (2.5 g, 49.940 mmol) were successively added to a stirred solution of rac-(5-bromopyridin-2-yl)(tetrahydrofuran-3-yl)methanone (5 g, 19.524 mmol) in ethylene glycol (30 mL). The reaction mixture was heated and stirred at 130° C. for 16 h. The mixture was partitioned between ethyl acetate (60 mL) and water (25 mL). The aqueous layer was separated and extracted with ethyl acetate (60 mL). The combined organic extracts were dried (MgSO₄), filtered and concentrated in vacuo. Purification by flash chromatography (SiO₂, 3 to 25% ethyl acetate in hexanes) gave rac-5-bromo-2-((tetrahydrofuran-3-yl)methyl)pyridine (1.2 g, 23%) as a light yellow oil. H NMR (400 MHz, Chloroform-d) δ 8.58 (d, J=2.24 Hz, 1H), 7.71 (dd, J=8.24, 2.36 Hz, 1H), 7.04 (d, J=8.28 Hz, 1H), 3.91-3.85 (m, 2H), 3.78-3.73 (m, 1H), 3.73-3.45 (m, 1H), 2.83 (d, J=2.08 Hz, 1H), 2.81 (s, 1H), 2.72-2.66 (m, 1H), 2.04-1.96 (m, 1H), 1.66-1.58 (m, 1H) ppm. ESI-MS m/z calc. 241.0102, found 242.0 (M+1)⁺; Retention time: 2.3 minutes.

### Intermediate AV

### 5-bromo-2-((tetrahydro-2H-pyran-4-yl)methyl)pyridine

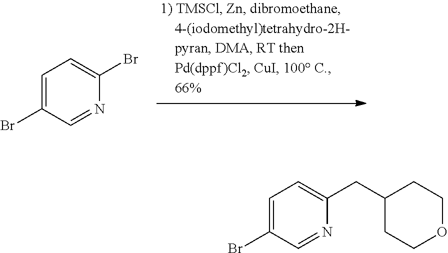 [SMILES: Brc1ccc(Br)nc1 ; Brc1ccc(CC2CCOCC2)nc1]

### Step 1:

TMSCl (256.80 mg, 0.3 mL, 2.364 mmol) and 1,2-dibromoethane (654.0 mg, 0.3 mL, 3.481 mmol) were successively added at ambient temperature to a stirred suspension of zinc dust (1.9 g, 29.056 mmol) freshly activated by heating with hot gun under high vacuum in DMA (5 mL). The reaction mixture was stirred under an argon atmosphere for 10 min. A solution of 4-(iodomethyl)tetrahydro-2H-pyran (3 g, 13.271 mmol) in DMA (5 mL) was added dropwise to the mixture. The reaction mixture was stirred for 2 h at ambient temperature. This resulting mixture was added via cannula to a stirred and degassed mixture of 2,5-dibromopyridine (1.6 g, 6.754 mmol), Pd(dppf)Cl₂ (1 g, 1.367 mmol) and CuI (520 mg, 2.730 mmol) in DMA (15 mL). The mixture was degassed again with argon and heated at 100° C. for 2 h. The reaction mixture was cooled down to ambient temperature, quenched by addition of a saturated aqueous NH₄Cl solution (50 mL) and passed over a thin bed of Celite, washing with ethyl acetate (200 mL). The filtrates were diluted with ethyl acetate (100 mL). The organic layer was separated and washed with water and brine, dried (Na₂SO₄), filtered and concentrated in vacuo. Purification by flash chromatography (SiO₂, 10 to 15% ethyl acetate in hexanes) gave 5-bromo-2-((tetrahydro-2H-pyran-4-yl)methyl)pyridine (870 mg, 26%) as a colorless oil. ¹H NMR (400 MHz, DMSO-d₆) δ 8.60 (d, J=1.52 Hz, 1H), 7.94-7.92 (m, 1H), 7.24 (d, J=8.24 Hz, 1H), 3.79 (d, J=9.12 Hz, 2H), 3.31-3.20 (m, 2H), 2.64 (d, J=7.08 Hz, 2H), 1.96-1.91 (m, 1H), 1.44 (d, J=12.0 Hz, 2H), 1.26-1.17 (m, 2H) ppm. ESI-MS m/z calc. 255.0259, found 256.0 (M+1)⁺; Retention time: 2.78 minutes.

### Intermediate AW

### 1-(4-bromopyridin-2-yl)-2-methylpropan-2-ol

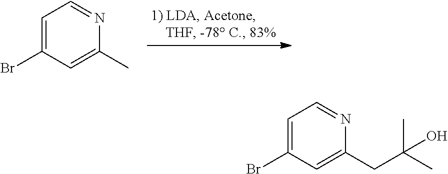

### Step 1:

LDA (3.5 mL, 2 M solution in THF, heptane and ethylbenzene, 7.0 mmol) was added dropwise to a stirred solution of 4-bromo-2-methyl-pyridine (801 mg, 4.656 mmol) in THF (10 mL) cooled to −78° C. The mixture was stirred at −78° C. for 15 min then acetone (690 μL, 9.397 mmol) was added dropwise. The reaction was stirred at −78° C. for 45 min before being quenched by addition of a saturated aqueous ammonium chloride solution. The aqueous layer was separated and extracted with DCM (3×10 mL). The combined organic layers were dried (MgSO₄), filtered and concentrated in vacuo. Purification by flash chromatography (24 g SiO₂, 0 to 100% EtOAc in heptane) gave 1-(4-bromopyridin-2-yl)-2-methylpropan-2-ol (891 mg, 83%). ESI-MS m/z calc. 229.01022, found 232.0 (M+1)⁺; Retention time: 0.59 minutes.

The following intermediate was made using a method similar to that described in Intermediate AW except that, 5-bromo-2-methylpyridine was used as starting material in place of 4-bromo-2-methyl-pyridine:

| | | | |
|---|---|---|---|
| Inter- | | | NMR |
| mediate | | | (shifts |
| No. | Compound Name | LC/MS | in ppm) |

| | | |
|---|---|---|
| AX | 1-(5-bromopyridin- | ESI-MS m/z calc. |
| | 2-yl)-2- | 229.01022, found 232.0 |
| | methylpropan-2-ol | (M + 1)⁺; Retention |
| | | time: 0.59 minutes |

### Intermediate AY

### rac-6-bromo-2,3-dihydrofuro[3,2-b]pyridin-3-ol

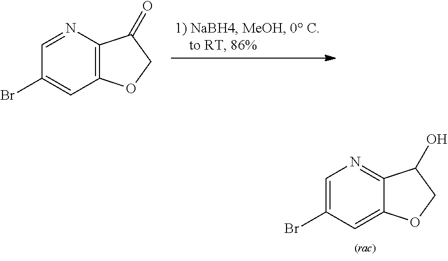 [SMILES: O=C1COc2cc(Br)cnc21 ; OC1COc2cc(Br)cnc21]

### Step 1:

6-Bromofuro[3,2-b]pyridin-3(2H)-one (Hydrochloride salt) (300 mg, 1.198 mmol) was suspended in MeOH (5.0 mL) and cooled to 0° C. NaBH₄ (113 mg, 2.987 mmol) was added portionwise, causing some effervescence. The reaction mixture was allowed to warm to ambient temperature and was stirred for 2 h. The mixture was quenched by pouring the solution over water (15 mL). The mixture was diluted with EtOAc (15 mL). The aqueous layer was separated and extracted with EtOAc (2×15 mL). The combined organic extracts were washed with brine (20 mL), dried (MgSO₄), filtered and concentrated in vacuo to give rac-6-bromo-2,3-dihydrofuro[3,2-b]pyridin-3-ol (223 mg, 86%) as an orange solid. ¹H NMR (500 MHz, DMSO-d₆) δ 8.21 (d, J=1.9 Hz, 1H), 7.62 (d, J=1.9 Hz, 1H), 5.95 (d, J=5.7 Hz, 1H), 5.16-5.12 (m, 1H), 4.68 (dd, J=10.4, 7.0 Hz, 1H), 4.36 (dd, J=10.4, 3.0 Hz, 1H) ppm. ESI-MS m/z calc. 214.95819, found 216.1 (M+1)⁺; Retention time: 1.57 minutes.

### Intermediate AZ

### tert-butyl rac-(1-(5-bromopyridin-2-yl)-2-methoxyethyl)(methyl)carbamate

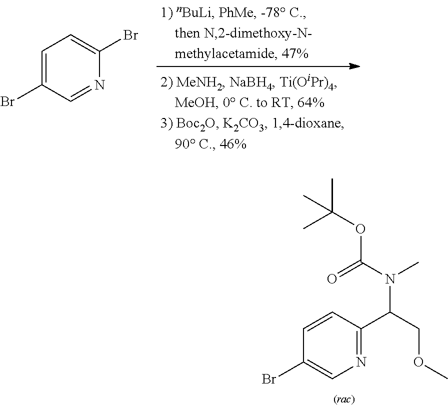 [SMILES: Brc1ccc(Br)nc1 ; COCC(c1ccc(Br)cn1)N(C)C(=O)OC(C)(C)C]

### Step 1:

ⁿBuLi (50 mL, 2 M solution, 100.0 mmol) was added to a stirred solution of 2,5-dibromopyridine (20 g, 84.427 mmol) in toluene (600 mL) at −78° C. The mixture was stirred for 45 min at −78° C. A solution of N,2-dimethoxy-N-methylacetamide (13.5 g, 101.39 mmol) in toluene (100 mL) was added to the reaction mixture at −78° C. The reaction was stirred for 30 min at −78° C. The mixture was quenched by addition of an aqueous saturated ammonium chloride solution (300 mL). The aqueous phase was separated and extracted with ethyl acetate (2×500 mL). The combined organic extracts were washed with brine (300 mL), dried (Na₂SO₄) and concentrated in vacuo. Purification by flash chromatography (SiO₂, 10 to 15% ethyl acetate in hexanes) gave 1-(5-bromopyridin-2-yl)-2-methoxyethan-1-one (9.1 g, 47%) as a light yellow solid. ¹H NMR (400 MHz, DMSO-d₆) δ 8.84 (s, 1H), 8.30-8.28 (m, 1H), 7.88 (d, J=8.3 Hz, 1H), 4.94 (s, 2H), 3.38 (s, 3H) ppm. ESI-MS m/z calc. 228.9738, found 200.0 (M−30)⁺; Retention time: 1.63 minutes.

### Step 2:

Methyl amine (81 mg, 2.608 mmol) and titanium isopropoxide (3.7 g, 13.018 mmol) were successively added to a stirred solution of 1-(5-bromopyridin-2-yl)-2-methoxyethan-1-one (600 mg, 2.608 mmol) in methanol (15 mL) at 0° C. The reaction mixture was stirred at ambient temperature for 1 h. NaBH₄ (296 mg, 7.824 mmol) was added at 0° C. and reaction mixture was slowly warmed up to ambient temperature with stirring for 16 h. The reaction mixture was concentrated in vacuo. Purification by flash chromatography (SiO₂, 5% methanol in ethyl acetate) gave rac-1-(5-bromopyridin-2-yl)-2-methoxy-N-methylethan-1-amine (410 mg, 64%) as a colourless oil. ¹H NMR (400 MHz, DMSO-d₆) δ 8.62 (d, J=2 Hz, 1H), 8 (dd, J=8.32 Hz, 2.24 Hz, 1H), 7.41 (d, J=8.36 Hz, 1H), 3.75-3.72 (m, 1H), 3.49-3.40 (m, 2H), 3.20 (s, 3H), 3.16 (s, 1H), 2.15 (s, 3H) ppm. ESI-MS m/z calc. 244.0211, found 245.06 (M+1)⁺; Retention time: 1.09 minutes.

### Step 3:

K₂CO₃ (812 mg, 5.875 mmol) and Boc₂0 (665 mg, 0.7 mL, 3.047 mmol) were successively added to a stirred solution of rac-1-(5-bromopyridin-2-yl)-2-methoxy-N-methylethan-1-amine (480 mg, 1.958 mmol) in 1,4-dioxane (4.8 mL) at ambient temperature. The reaction mixture was stirred at 90° C. for 3 h. The reaction mixture was diluted with H₂O and extracted with DCM (2×200 mL). The combined organic extracts were washed with brine (200 mL). The organic layer was dried (MgSO₄), filtered and concentrated in vacuo. Purification by flash chromatography (SiO₂, 5 to 15% ethyl acetate in hexanes) gave tert-butyl rac-(1-(5-bromopyridin-2-yl)-2-methoxyethyl)(methyl)carbamate (320 mg, 46%) as a colourless oil. ¹H NMR (400 MHz, DMSO-d₆) δ 8.65 (d, J=1.92 Hz, 1H), 8.04 (d, J=7.76 Hz, 1H), 7.25 (d, J=8.28 Hz, 1H), 5.38 (s, 0.5H), 5.17 (s, 0.5H), 3.88-3.79 (m, 2H), 3.31 (d, J=10.08 Hz, 3H), 2.73 (d, J=10.72 Hz, 3H), 1.39 (d, J=59.4 Hz, 9H) ppm. ESI-MS m/z calc. 344.0736, found 345.1 (M+1)⁺; Retention time: 2.82 minutes.

The following intermediate was made using a method similar to that described in Intermediate AZ except that, in Step 2, azetidine was used in place of methyl amine and the reaction was carried out in dichloroethane rather than methanol as the solvent. Step 3 was omitted:

| | | | |
|---|---|---|---|
| Inter- | | | NMR |
| mediate | | | (shifts |
| No. | Compound Name | LC/MS | in ppm) |

| | | |
|---|---|---|
| BA | rac-2-(1-(azetidin- | ESI-MS m/z calc. |
| | 1-y1)-2- | 270.0368, found 272.0 |
| | methoxyethyl)-5- | (M + 1)⁺; Retention |
| | bromopyridine | time: 2.23 minutes |

The following intermediates were made using a method similar to that described in Intermediate 10 except that, in Step 2, different amines were used in place of methyl amine:

| Intermediate | | | |
|---|---|---|---|
| No. | Compound Name | LC/MS | NMR (shifts in ppm) |
| BB | tert-butyl (1-(5-bromopyridin- | ESI-MS m/z calc. | ¹H NMR (400 MHz, |
| | 2-yl)-2-methoxyethyl)(2- | 388.0998, found 389.1 | DMSO-d₆) δ 8.61 (d, J = |
| | methoxyethyl)carbamate | (M + 1)⁺; Retention | 2.08 Hz, 1H), 7.99-7.96 |
| | | time: 2.84 minutes | (m, 1H), 7.31 (d, J = 8.4 |
| | | | Hz, 1H), 5.09 (t, J = 6.96 |
| | | | Hz, 1H), 4.03-3.99 (m, |
| | | | 1H), 3.89-3.84 (m, 1H), |
| | | | 3.89-3.37 (m, 4H), 3.32 |
| | | | (s, 3H), 3.21 (s, 3H), 1.36 |
| | | | (s, 9H) ppm. |
| BC | tert-butyl rac-(1-(5- | ESI-MS m/z calc. | ¹H NMR (400 MHz, |
| | bromopyridin-2-y1)-2- | 372.1049, found 373.1 | DMSO-d₆) δ 8.60 (d, J = |
| | methoxyethyl)(isopropyl)carbamate | (M + 1)⁺; Retention | 2.24 Hz, 1H), 8.02-7.96 |
| | | time: 3.09 minutes | (m, 1H), 7.34 (t, J = |
| | | | 11.04 Hz, 1H), 4.97- |
| | | | 4.94 (m, 1H), 4.09-4.05 |
| | | | (m, 1H), 3.86-3.82 (m, |
| | | | 2H), 3.34 (s, 3H), 1.36 |
| | | | (s, 9H), 1.28-1.05 (m, 6H) |
| | | | ppm. |

The following intermediate was made using a method similar to that described in Intermediate AZ except that the conditions of Step 1 were those described in Intermediate 11 Step 1. In Step 3, triethylamine was used in place of K₂CO₃ and the reaction was carried out at ambient temperature in methanol rather than 1,4-dioxane as the solvent:

| | | | |
|---|---|---|---|
| | | | NMR |
| Interme- | | | (shifts |
| diate No. | Compound Name | LC/MS | in ppm) |

| | | |
|---|---|---|
| BD | tert-butyl rac-(1-(4-chloro-5- | ESI-MS m/z calc. |
| | fluoropyridin-2-yl)-2- | 318.11465, found |
| | methoxyethyl)(methyl)carbamate | 263.1 |
| | | (M − 55)⁺; |
| | | Retention time: |
| | | 0.95 minutes |

### Intermediate BE

### tert-butyl rac-(1-(4-chloro-5-fluoropyridin-2-yl)-2-methoxyethyl)carbamate

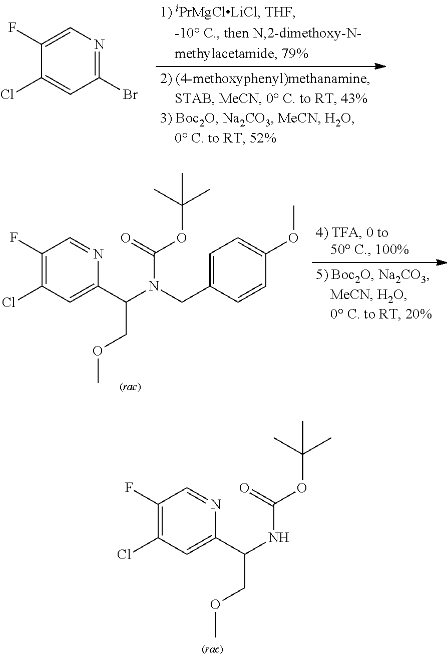 [SMILES: Fc1cnc(Br)cc1Cl ; COCC(c1cc(Cl)c(F)cn1)N(Cc1ccc(OC)cc1)C(=O)OC(C)(C)C ; COCC(NC(=O)OC(C)(C)C)c1cc(Cl)c(F)cn1]

### Step 1:

A solution of ⁱPrMgCl·LiCl (238 mL, 1.3 M in THF, 309.4 mmol) in THF (400 mL) was cooled down to −10° C. internally (acetone cardice bath). A solution of 2-bromo-4-chloro-5-fluoropyridine (50 g, 237.6 mmol) in THF (170 mL) was added at such a rate to keep the internal temperature below −5° C. The mixture was stirred between -7 and −5° C. for 30 min. The reaction mixture, a dark brown solution, was cooled back down to −10° C. internally. A solution of N,2-dimethoxy-N-methylacetamide (38.1 g, 271.8 mmol) in THF (150 mL) was added over 30 min at such a rate to keep the internal temperature below −8° C. After the end of addition, the cooling bath was removed and the mixture was warmed ambient temperature. The mixture was quenched by addition of a saturated aqueous NH₄Cl solution (250 mL), causing an exotherm to 27.8° C. Water (250 mL) was added to help the dissolution of salts. The organic phase was separated, dried (MgSO₄), filtered and concentrated in vacuo to give a brown solid. Purification by flash chromatography (750 g SiO₂, 0 to 20% EtOAc in heptane) 1-(4-chloro-5-fluoropyridin-2-yl)-2-methoxyethan-1-one (38.2 g, 79%) as a cream solid. ¹H NMR (400 MHz, DMSO-d₆) δ 8.84 (d, J=1.1 Hz, 1H), 8.18 (d, J=6.1 Hz, 1H), 4.93 (s, 2H), 3.38 (s, 3H) ppm. ESI-MS m/z calc. 203.01494, Retention time: 0.63 minutes.

### Step 2:

(4-Methoxyphenyl)methanamine (2.843 g, 2.7 mL, 20.725 mmol) was added to a stirred solution of 1-(4-chloro-5-fluoropyridin-2-yl)-2-methoxyethan-1-one (4 g, 19.647 mmol) in MeCN (40 mL) at ambient temperature under argon. The reaction mixture was stirred for 25 min. Sodium triacetoxyborohydride (12.5 g, 58.979 mmol) was added portionwise at 0° C. over 30 min and the reaction was stirred overnight at ambient temperature. The mixture was concentrated in vacuo. The residue was partitioned between ethyl acetate and water. The organic phase was separated, dried (Na₂SO₄), filtered and concentrated in vacuo to give rac-1-(4-chloro-5-fluoropyridin-2-yl)-2-methoxy-N-(4-methoxybenzyl)ethan-1-amine (6.3 g, 43%), which was used in the next step without further purification. ESI-MS m/z calc. 324.1041, found 325.0 (M+1)⁺; Retention time: 1.46 minutes.

### Step 3:

A solution of Na₂CO₃ (7.3 g, 68.876 mmol) in H₂O (8 mL) was added to a stirred solution of rac-1-(4-chloro-5-fluoropyridin-2-yl)-2-methoxy-N-(4-methoxybenzyl)ethan-1-amine (3.7 g, 11.392 mmol) in MeCN (25 mL) under argon. The reaction mixture was stirred for 15 min. Boc₂0 (5.035 g, 5.3 mL, 23.070 mmol) was added to the reaction mixture at 0° C. and the mixture was stirred for 16 h at ambient temperature. The mixture was partitioned between ethyl acetate (500 mL) and water. The organic phase was separated, washed with brine, dried (Na₂SO₄), filtered and concentrated in vacuo. Purification by flash chromatography (SiO₂, 10 to 30% ethyl acetate in hexanes) gave tert-butyl rac-(1-(4-chloro-5-fluoropyridin-2-yl)-2-methoxyethyl)(4-methoxybenzyl)carbamate (2.5 g, 52%) as a yellow oil. ¹H NMR (DMSO-d₆) δ 8.60 (s, 1H), 7.45-6. 79 (m, 5H), 5.39 (br s, 0.5H), 5.02 (br s, 0.5H), 4.33-3.92 (m, 4H), 3.70 (s, 3H), 3.18 (s, 3H), 1.29 (s, 9H) ppm.

### Step 4:

TFA (19.240 g, 13 mL, 168.74 mmol) was added to tert-butyl rac-(1-(4-chloro-5-fluoropyridin-2-yl)-2-methoxyethyl)(4-methoxybenzyl)carbamate (3.5 g, 8.237 mmol) at 0° C. under argon and the mixture was stirred for 10 min. The mixture was warmed to ambient temperature and heated at 50° C. for 120 h. The reaction mixture was concentrated in vacuo to give rac-1-(4-chloro-5-fluoropyridin-2-yl)-2-methoxyethan-1-amine (3.3 g, 100%) as brown oil, which was used without further purification in the next step. ESI-MS m/z calc. 204.0466, found 205.0 (M+1)⁺; Retention time: 1.19 minutes.

### Step 5:

A solution of Na₂CO₃ (10.3 g, 97.181 mmol) in H₂O (15 mL) was added to a stirred solution of rac-1-(4-chloro-5-fluoropyridin-2-yl)-2-methoxyethan-1-amine (3.3 g, 16.127 mmol) in MeCN (30 mL). The mixture was stirred for 15 min. Boc₂0 (7.125 g, 7.5 mL, 32.646 mmol) was added to the mixture at 0° C. and the resulting mixture was stirred for 16 h at ambient temperature. The reaction mixture was partitioned between ethyl acetate (600 mL) and water. The organic layer was separated, washed with brine, dried (Na₂SO₄), filtered and concentrated in vacuo. Purification by flash chromatography (SiO₂, 2 to 15% ethyl acetate in DCM) gave tert-butyl rac-(1-(4-chloro-5-fluoropyridin-2-yl)-2-methoxyethyl)carbamate (1.1 g, 20%) as a brown oil. ¹H NMR (DMSO-d₆) δ 8.65 (s, 1H), 7.64 (d, J=8 Hz, 1H), 7.36 (d, J=8 Hz, 1H), 4.81-4.77 (m, 1H), 3.53 (d, J=4 Hz, 2H), 3.22 (s, 3H), 1.37 (s, 9H) ppm.

The following intermediate was made using a method similar to that described in Intermediate BE except that, in Step 1, 2-(benzyloxy)-N-methoxy-N-methylacetamide was used in place of N,2-dimethoxy-N-methylacetamide and the reaction was carried out in toluene in place of THF. In Step 2, methyl amine was used in place of (4-methoxyphenyl)methanamine. In Step 3, triethylamine was used in place of Na₂CO₃ and the reaction was carried out at ambient temperature in methanol rather than 1,4-dioxane as the solvent. Steps 4 and 5 were omitted:

| Intermediate | | | |
|---|---|---|---|
| No. | Compound Name | LC/MS | NMR (shifts in ppm) |
| BF | tert-butyl rac-(2-(benzyloxy)-1- | ESI-MS m/z calc. | ¹H NMR (400 MHz, |
| | (4-chloro-5-fluoropyridin-2- | 394.1459, found 395.2 | DMSO-d₆) δ 8.66 (s, 1H), |
| | yl)ethyl)(methyl)carbamate | (M + 1)⁺; Retention time: | 7.56 (d, J = 4 Hz, 1H), |
| | | 2.76 | 7.36-7.26 (m, 5H), 5.46 |
| | | | (br s, 0.5 H), 5.20 (br s, |
| | | | 0.5 H), 4.60-4.50 (m, |
| | | | 2H), 3.99-3.88 (m, |
| | | | 2H), 2.78 (s, 1.5H), 2.73 |
| | | | (s, 1.5 H), 1.40 (s, 4.5H), |
| | | | 1.27 (s, 4.5H) ppm. |

### Intermediate BG

### tert-butyl rac-(1-(4-bromopyridin-2-yl)-2-methoxyethyl)(methyl)carbamate

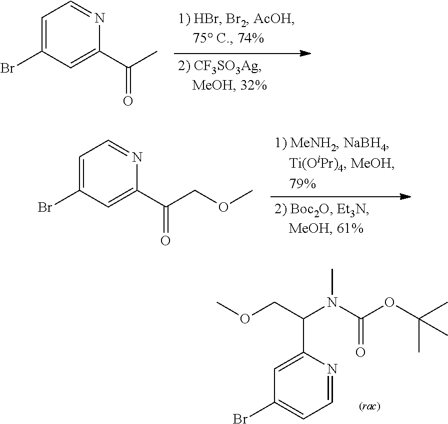

### Step 1:

Hydrogen bromide (715 μL, 33% w/w in acetic acid, 4.345 mmol) and Br₂ (190 μL, 3.688 mmol) were successively added slowly to a stirred solution of 1-(4-bromopyridin-2-yl)ethan-1-one (715 mg, 3.574 mmol) in AcOH (5 mL) at ambient temperature. The reaction mixture was heated at 75° C. for 45 min then cooled to 0° C. A saturated aqueous NaHCO₃ solution (30 mL) was added slowly with stirring followed by solid NaHCO₃ to adjust the pH to ˜7. The mixture was extracted with EtOAc (4×10 mL). The combined organic extracts were dried (MgSO₄), filtered and concentrated in vacuo to give 2-bromo-1-(4-bromopyridin-2-yl)ethan-1-one (1.132 g, 74%) as a brown oil (65% purity, contaminated with dibromo side product). ¹H NMR (500 MHz, Chloroform-d) δ 8.53 (dd, J=5.2, 0.6 Hz, 1H), 8.27 (dd, J=1.9, 0.6 Hz, 1H), 7.71 (dd, J=5.2, 1.9 Hz, 1H), 4.82 (s, 2H) ppm. ESI-MS m/z calc. 276.87378, found 280.2 (M+1)⁺; Retention time: 0.79 minutes.

### Step 2:

A mixture of 2-bromo-1-(4-bromopyridin-2-yl)ethan-1-one (4.75 g, 10.39 mmol) and silver triflate (7.50 g, 29.19 mmol) in MeOH (100 mL) was stirred for 16 h at ambient temperature. A further amount of silver triflate (2.5 g, 9.730 mmol) was added and the reaction mixture was stirred for 128h at ambient temperature. The MeOH was removed in vacuo and the residue was extracted with EtOAc (3×100 mL). The combined organic phases were dried (MgSO₄) and concentrated in vacuo. Purification by flash chromatography (40 g SiO₂, 0 to 100% EtOAC in heptane) gave 1-(4-bromopyridin-2-yl)-2-methoxyethan-1-one (762 mg, 32%) as an off white solid. ESI-MS m/z calc. 228.97385, found 230.1 (M+1)⁺; Retention time: 0.6 minutes.

### Step 3:

1-(4-Bromopyridin-2-yl)-2-methoxyethan-1-one (650 mg, 2.825 mmol) was added to a stirred solution of methylamine (4.3 mL, 2 M in MeOH, 8.6 mmol) and titanium isopropoxide (1.1 mL, 3.727 mmol) in MeOH (0.5 mL). The reaction mixture was stirred at ambient temperature for 5 h. NaBH₄ (128 mg, 3.383 mmol) was added and the stirring was continued overnight at ambient temperature. A further amount of sodium borohydride (50 mg, 1.322 mmol) was added and the reaction was stirred at ambient temperature for 40 h. Water (5 mL) was added to the mixture which was stirred for a further 7 h. The mixture was filtered through a pad of Celite, rinsing with water (10 mL) and EtOAc (10 mL). The aqueous phase was separated and 1M NaOH (10 mL) was added. The aqueous phase was extracted with EtOAc (2×30 mL). The combined organic extracts were dried (MgSO₄), filtered and concentrated in vacuo to give rac-1-(4-bromopyridin-2-yl)-2-methoxy-N-methylethan-1-amine (630 mg, 79%) as an orange oil. ESI-MS m/z calc. 244.02112, found 245.1 (M+1)⁺; Retention time: 0.54 minutes.

### Step 4:

Boc₂O (565 μL, 2.459 mmol) was added to a stirred solution of rac-1-(4-bromopyridin-2-yl)-2-methoxy-N-methylethan-1-amine (630 mg, 2.236 mmol) and triethylamine (343 μL, 2.461 mmol) in MeOH (15 mL). The reaction mixture was stirred at ambient temperature for 4 h 30. The reaction mixture was concentrated in vacuo. The residue was dissolved in EtOAc (10 mL), washed with water (10 mL) and brine (10 mL). The organic phases were passed through a phase separator cartridge and the filtrate was concentrated in vacuo. Purification by flash chromatography (12 g SiO₂, 0 to 22% EtOAc in heptane) gave tert-butyl rac-(1-(4-bromopyridin-2-yl)-2-methoxyethyl)(methyl)carbamate (498 mg, 61%). ESI-MS m/z calc. 344.07355, found 347.2 (M+1)⁺; Retention time: 0.9 minutes.

### Intermediate BH

### tert-butyl rac-(2-((tert-butyldimethylsilyl)oxy)-1-(4-chloro-5-fluoropyridin-2-yl)-2-methylpropyl)carbamate

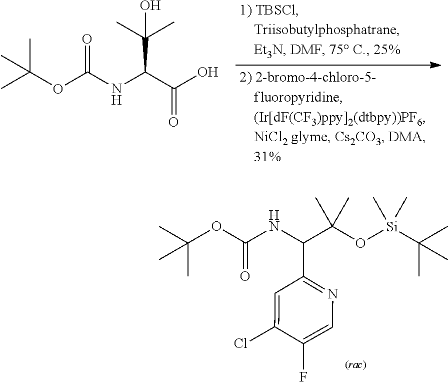 [SMILES: CC(C)(C)OC(=O)N[C@H](C(=O)O)C(C)(C)O ; CC(C)(C)OC(=O)NC(c1cc(Cl)c(F)cn1)C(C)(C)O[Si](C)(C)C(C)(C)C]

### Step 1:

TBSCl (526 μL, 2.827 mmol) was added to a solution of (S)-2-((tert-butoxycarbonyl)amino)-3-hydroxy-3-methylbutanoic acid (300 mg, 1.286 mmol), triisobutylEtOAcphosphatrane (91 μL, 0.2561 mmol) and triethylamine (537 μL, 3.853 mmol) in DMF (3 mL). The reaction mixture was heated to 75° C. for 48 h. The mixture was cooled to ambient temperature and 1 M HCl (7.7 mL, 7.700 mmol) was added with rapid stirring. The mixture was partitioned between MTBE (20 mL) and water (20 mL). The aqueous phase was separated and extracted with MTBE (10 mL). The combined organic extracts were washed with brine (1×10 mL), dried (MgSO₄) and concentrated in vacuo. Purification by flash chromatography (12 g SiO₂, 0 to 100% EtOAc in heptane) gave (S)-2-((tert-butoxycarbonyl)amino)-3-((tert-butyldimethylsilyl)oxy)-3-methylbutanoic acid (110 mg, 25%) as an oil. ESI-MS m/z calc. 347.2128, found 248.2 (M-Boc)⁺; Retention time: 0.56 minutes.

### Step 2:

In a 25 mL vial, a mixture of 2-bromo-4-chloro-5-fluoropyridine (400 mg, 1.901 mmol), NiCl₂ glyme (20 mg, 0.09102 mmol), 4-tert-butyl-2-(4-tert-butyl-2-pyridyl)pyridine (38 mg, 0.1416 mmol), (Ir[dF(CF₃)ppy]₂(dtbpy))PF₆ (42 mg, 0.037 mmol), cesium carbonate (928 mg, 2.848 mmol) and (S)-2-((tert-butoxycarbonyl)amino)-3-((tert-butyldimethylsilyl)oxy)-3-methylbutanoic acid (990 mg, 2.849 mmol) in DMA (15 mL) was flushed with nitrogen for 1 min and placed in a Penn OC Photoreactor M2 (100% LED, 500 rpm stirring rate) for 12 h. The reaction mixture was partitioned between ethyl acetate (30 mL) and water (30 mL). The aqueous layer was separated and extracted with EtOAc (50 mL). The combined organic extracts were washed with brine (20 mL), dried (Na₂SO₄), filtered and concentrated in vacuo. Purification by flash chromatography (SiO₂, 0 to 100% EtOAc in hexanes) gave tert-butyl rac-(2-((tert-butyldimethylsilyl)oxy)-1-(4-chloro-5-fluoropyridin-2-yl)-2-methylpropyl)carbamate (400 mg, 31%) as an oil. ESI-MS m/z calc. 432.2011, found 433.3 (M+1)⁺; Retention time: 1.35 minutes.

### Intermediate BI

### 7-bromo-2-(2-((triisopropylsilyl)oxy)ethyl)-[1,2,4]triazolo[4,3-a]pyridin-3(2H)-one

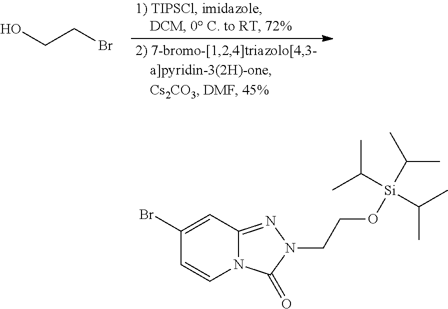

### Step 1:

Imidazole (1.1 g, 16.158 mmol) and triisopropylsilyl chloride (2.433 g, 2.7 mL, 12.618 mmol) were successively added to a stirred solution of 2-bromoethanol (1.3 g, 10.403 mmol) in DCM (20 mL) at ice cold temperature. The reaction mixture was stirred for 16 h at ambient temperature. Water (100 mL) was added to the mixture. The organic layer was separated, dried (MgSO₄), filtered and concentrated in vacuo. Purification by flash chromatography (SiO₂, 0 to 20% EtOAc in hexanes) gave (2-bromoethoxy)triisopropylsilane (2.1 g, 72%) as colourless oil. ¹H NMR (300 MHz, Chloroform-d) δ 3.99 (t, J=6.7 Hz, 2H), 3.44 (t, J=6.7 Hz, 2H), 1.21-1.01 (m, 21H) ppm.

### Step 2:

A suspension of 7-bromo-[1,2,4]triazolo[4,3-a]pyridin-3(2H)-one (700 mg, 3.271 mmol) and Cs2CO3 (3.8 g, 11.663 mmol) in DMF (10 mL) was stirred for 15 min at 0° C. (2-bromoethoxy)triisopropylsilane (1.1 g, 3.910 mmol) was added and the mixture was stirred for 24 h at ambient temperature. The mixture was quenched by addition of ice water. The aqueous phase was separated and extracted with ethyl acetate. The combined organic extracts were washed with brine and concentrated in vacuo. Purification by flash chromatography (SiO₂, 0 to 30% EtOAc in hexanes) gave 7-bromo-2-(2-((triisopropylsilyl)oxy)ethyl)-[1,2,4]triazolo[4,3-a]pyridin-3(2H)-one (610 mg, 45%) as a light yellow solid. ¹H NMR (400 MHz, DMSO-d₆) δ 7.81 (d, J=7.3 Hz, 1H), 7.66 (s, 1H), 6.70 (d,d, J=1.4 Hz, 7.3 Hz, 1H), 3.97 (s, 4H), 0.95-0.91 (m, 21H) ppm. ESI-MS m/z calc. 413.1134, found 414.0 (M+1)⁺; Retention time: 2.29 minutes.

The following intermediates were made using a method similar to that described in Intermediate BI except that, the order in which Steps 1 and 2 were carried out has been reversed. In the case of BJ, in Step 1, rac-(1S,2R)-2-((4-bromopyridin-2-yl)oxy)cyclohexan-1-ol and tert-butylchlorodimethylsilane were used as starting materials in place of 2-bromoethanol and triisopropylsilyl chloride and in Step 2, 4-bromo-2-fluoropyridine and rac-(1S,2R)-cyclohexane-1,2-diol were used as starting materials in place of 7-bromo-[1,2,4]triazolo[4,3-a]pyridin-3(2H)-one and (2-bromoethoxy)triisopropylsilane. In the case of BK, in Step 1, rac-(3S,4R)-4-((5-bromopyridin-3-yl)oxy)tetrahydrofuran-3-ol and tert-butylchlorodimethylsilane were used as starting materials in place of 2-bromoethanol and triisopropylsilyl chloride and the reaction was carried out in DMF as the solvent and in the presence of a catalytic amound of DMAP. In Step 2, 3-bromo-5-fluoropyridine and rac-(3R,4S)-tetrahydrofuran-3,4-diol were used as starting materials in place of 7-bromo-[1,2,4]triazolo[4,3-a]pyridin-3(2H)-one and (2-bromoethoxy)triisopropylsilane:

| Intermediate | | | |
|---|---|---|---|
| No. | Compound Name | LC/MS | NMR (shifts in ppm) |
| BJ | rac-4-bromo-2-(((1S,2R)-2- | ESI-MS m/z calc. | ¹H NMR (400 MHz, |
| | ((tert- | 385.1073, found 386.1 | DMSO-d₆) δ 8.04 (d, J = |
| | butyldimethylsilyl)oxy)cyclohexyl)oxy)pyridine | (M + 1)⁺; Retention time: | 5.4 Hz, 1H), 7.19-7.17 |
| | | 3.27 minutes | (m, 1H), 6.97 (s, 1H), |
| | | | 5.01 (d, J = 9.3 Hz, 1H), |
| | | | 4.13 (s, 1H), 1.84-1.81 |
| | | | (m, 1H), 1.70-1.62 (m, |
| | | | 3H), 1.57-1.51 (m, 2H), |
| | | | 1.35-1.33 (m, 2H), 0.83 |
| | | | (s, 9H), −0.075 (d, J = |
| | | | 14.6 Hz, 6H) ppm. |
| BK | rac-3-bromo-5-(((3R,4S)-4- | ESI-MS m/z calc. | ¹H NMR (400 MHz, |
| | ((tert- | 373.0709, found 374.0 | DMSO-d₆) δ 8.30 (d, 2.4 |
| | butyldimethylsilyl)oxy)tetrahydrofuran- | (M + 1)⁺; Retention time: | Hz, 1H), 8.24 (s, 1H), |
| | 3-yl)oxy)pyridine | 2.15 minutes | 7.75 (s, 1H), 5.09 (dd, J = |
| | | | 4.76 Hz, J = 9.2 Hz, 1H), |
| | | | 4.60 (dd, J = 5.8 Hz, J = |
| | | | 11.16 Hz, 1H), 4.09-4.05 |
| | | | (dd, J = 5.44 Hz, 1H), |
| | | | 3.94 (d, J = 6.04 Hz, |
| | | | J = 8.44 Hz, 1H), 3.81 |
| | | | (dd, J = 3.84 Hz, J = |
| | | | 9.8 Hz, 1H), 3.52 (dd, J = |
| | | | 6.28 Hz, J = 8.36 Hz, |
| | | | 1H), 0.67 (s, 9H), −0.02 |
| | | | (s, 3H), −0.12 (s, 3H) |
| | | | ppm. |

### Intermediate BL

### 7-bromo-3-(((tert-butyldimethylsilyl)oxy)methyl)-[1,2,4]triazolo[4,3-a]pyridine

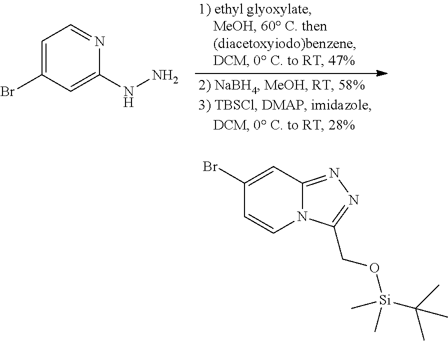

### Step 1:

Ethyl glyoxylate (10.5 mL, 50% w/v in toluene, 51.426 mmol) was added to a stirred solution of 4-bromo-2-hydrazinylpyridine (8 g, 42.548 mmol) in methanol (110 mL) and the mixture stirred at 60° C. for 2 h. The mixture was cooled down to ambient temperature and solvent was evaporated under reduced vacuum. The residue was dissolved in DCM (110 mL) and the mixture was cooled at 0° C. (Diacetoxyiodo)benzene (18 g, 55.884 mmol) added portionwise and the reaction mixture was stirred at ambient temperature for 12 h. Water (40 mL) was added. The organic layer was separated, dried (MgSO₄), filtered and concentrated in vacuo. Purification by flash chromatography (SiO₂, 20 to 75% EtOAc in hexanes) gave ethyl 7-bromo-[1,2,4]triazolo[4,3-a]pyridine-3-carboxylate (6.5 g, 47%) as a yellow solid. ¹H NMR (400 MHz, DMSO-d₆) δ 8.93 (d, J=7.36 Hz, 1H), 8.45 (s, 1H), 7.44-7.42 (m, 1H), 4.50-4.45 (m, 2H), 1.39 (t, J=8 Hz, 3H) ppm. ESI-MS m/z calc. 268.98, found 270.0 (M+1)⁺; Retention time: 1.58 minutes.

### Step 2:

NaBH₄ (3.4 g, 3.598 mL, 89.870 mmol) was added portionwise to a stirred solution of ethyl 7-bromo-[1,2,4]triazolo[4,3-a]pyridine-3-carboxylate (6 g, 22.215 mmol) in methanol (100 mL). The reaction mixture was stirred at ambient temperature for 16 h. The mixture was concentrated in vacuo and partitioned between DCM (50 mL) and water (20 mL). The organic layer was separated, dried (MgSO₄), filtered and concentrated in vacuo to give (7-bromo-[1,2,4]triazolo[4,3-a]pyridin-3-yl)methanol (3.2 g, 58%) as a yellow solid, which was used without further purification in the next step. ESI-MS m/z calc. 226.9694, found 228.2 (M+1)⁺; Retention time: 1.11 minutes.

### Step 3:

TBSCl (8 g, 53.078 mmol) and DMAP (320 mg, 2.619 mmol) were successively added to a stirred solution of (7-bromo-[1,2,4]triazolo[4,3-a]pyridin-3-yl)methanol (3 g, 13.155 mmol) and imidazole (2.7 g, 39.661 mmol) in DCM (50 mL) at 0° C. The reaction mixture was stirred for 16 h at ambient temperature. Water (15 mL) was added and the organic layer was separated, dried (MgSO₄), filtered and concentrated in vacuo. Purification by flash chromatography (SiO₂, 20 to 80% EtOAc in hexanes) gave 7-bromo-3-(((tert-butyldimethylsilyl)oxy)methyl)-[1,2,4]triazolo[4,3-a]pyridine (1.27 g, 28%) as a light yellow solid. ¹H NMR (400 MHz, DMSO-d₆) δ 8.32 (d, J=8 Hz, 1H), 8.19 (s, 1H), 7.21 (dd, J=1.76 Hz, 7.32 Hz, 1H), 5.19 (s, 2H), 0.81 (s, 9H), 0.04 (s, 6H) ppm. ESI-MS m/z calc. 341.0559, found 342.2 (M+1)⁺; Retention time: 3.24 minutes.

The following intermediate was made using a method similar to that described in Intermediate BL except that, in Step 1, 5-bromo-2-hydrazinylpyridine was used in place of 4-bromo-2-hydrazinylpyridine and the second part of the reaction was carried out in 1,4-dioxane in place of DCM:

| Intermediate | | | |
|---|---|---|---|
| No. | Compound Name | LC/MS | NMR (shifts in ppm) |
| BM | 6-bromo-3-(((tert- | ESI-MS m/z calc. | ¹H NMR (400 MHz, |
| | butyldimethylsilyl)oxy)methyl)- | 341.0559, found 344.17 | DMSO-d₆) δ 8.70 (s, 1H), |
| | [1,2,4]triazolo[4,3-a]pyridine | (M + 3)⁺; Retention time: | 7.82 (d, J = 9.64 Hz, 1H), |
| | | 2.08 minutes | 7.59-7.57 (m, 1H), 5.23 |
| | | | (s, 2H), 0.83 (s, 9H), 0.06 |
| | | | (s, 6H) ppm. |

### Intermediate BN

### rac-3-bromo-5-((tert-butyldimethylsilyl)oxy)-6,7-dihydro-5H-cyclopenta[b]pyridine

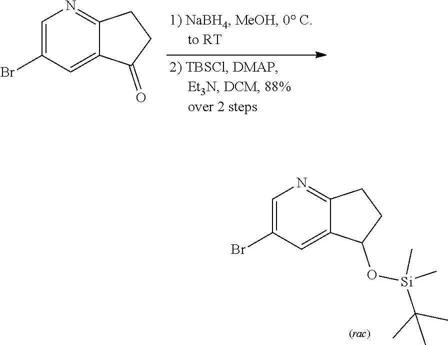 [SMILES: O=C1CCc2ncc(Br)cc21 ; CC(C)(C)[Si](C)(C)OC1CCc2ncc(Br)cc21]

### Step 1:

3-Bromo-6,7-dihydro-5H-cyclopenta[b]pyridin-5-one (200 mg, 0.9432 mmol) was suspended in MeOH (1.60 mL) and cooled to 0° C. NaBH₄ (43 mg, 1.137 mmol) was added portionwise causing effervescence. The reaction was allowed to warm to ambient temperature and was stir at ambient temperature for 1 h. The reaction mixture was poured over water (15 mL) and diluted with EtOAc (15 mL). The aqueous layer was separated and extracted with EtOAc (2×15 mL). The combined organic extracts washed with brine (20 mL), dried (MgSO₄), filtered and concentrated in vacuo to give rac-3-bromo-6,7-dihydro-5H-cyclopenta[b]pyridin-5-ol as a yellow oil, which was used without further purification in the next step. ESI-MS m/z calc. 212.97893, found 216.1 (M+1)⁺; 214.1 (M−1)⁻; Retention time: 0.46 minutes.

### Step 2:

TBSCl (285 mg, 1.891 mmol) and DMAP (22 mg, 0.1801 mmol) were successively added to a solution of rac-3-bromo-6,7-dihydro-5H-cyclopenta[b]pyridin-5-ol (201.9 mg, 0.943 mmol) and Et₃N (260 μL, 1.865 mmol) in DCM (4.0 mL). The reaction mixture was stirred at ambient temperature overnight. The mixture was diluted with DCM (20 mL) and poured over saturated aqueous NaHCO₃ (20 mL). The aqueous layer was separated and extracted with DCM (2×15 mL). The combined organic extracts were washed with brine (20 mL), dried (MgSO₄), filtered and concentrated in vacuo. Purification by flash chromatography (12 g SiO₂, 0 to 100% EtOAc in heptane) gave rac-3-bromo-5-((tert-butyldimethylsilyl)oxy)-6,7-dihydro-5H-cyclopenta[b]pyridine (273 mg, 88% over 2 steps) as a colourless oil. ¹H NMR (500 MHz, Chloroform-d) δ 8.48 (d, J=1.3 Hz, 1H), 7.65 (d, J=1.3 Hz, 1H), 5.24 (t, J=7.2 Hz, 1H), 3.04 (ddd, J=16.9, 9.1, 2.9 Hz, 1H), 2.88-2.80 (m, 1H), 2.53-2.46 (m, 1H), 2.03-1.95 (m, 1H), 0.95 (s, 9H), 0.18 (s, 3H), 0.16 (s, 3H) ppm. ESI-MS m/z calc. 327.0654, found 328.4 (M+1)⁺; Retention time: 4.02 minutes.

The following intermediate was made using a method similar to that described in Intermediate BN except that, Step 1 was omitted and rac-3-bromo-6,7-dihydro-5H-cyclopenta[b]pyridin-7-ol was used in place of rac-3-bromo-6,7-dihydro-5H-cyclopenta[b]pyridin-5-ol in Step 2:

| Intermediate | | | |
|---|---|---|---|
| No. | Compound Name | LC/MS | NMR (shifts in ppm) |
| BO | rac-3-bromo-7-((tert- | ESI-MS m/z calc. | ¹H NMR (500 MHz, |
| | butyldimethylsilyl)oxy)- | 327.0654, found 328.4 | Chloroform-d) δ 8.50-8.49 |
| | 6,7-dihydro-5H- | (M + 1)⁺; Retention time: | (m, 1H), 7.65-7.64 |
| | cyclopenta[b]pyridine | 4.15 minutes | (m, 1H), 5.10 (dd, J = 6.9, |
| | | | 4.9 Hz, 1H), 3.07-2.99 |
| | | | (m, 1H), 2.79-2.71 (m, |
| | | | 1H), 2.44-2.37 (m, 1H), |
| | | | 2.06-1.99 (m, 1H), 0.92 |
| | | | (s, 9H), 0.20 (s, 3H), 0.15 |
| | | | (s, 3H) ppm. |

### Intermediate BP

### rac-5-bromo-2-(2-(tert-butoxy)-1-fluoroethyl)pyridine

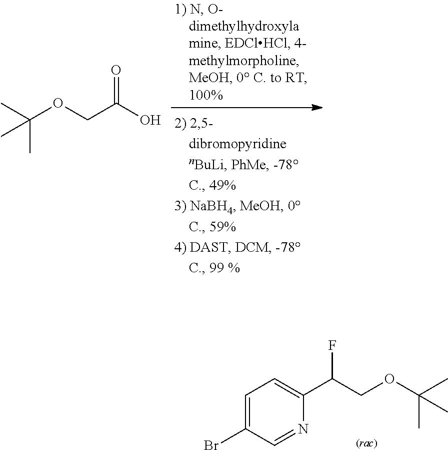

### Step 1:

EDCI hydrochloride (33 g, 172.14 mmol) was added in 5 portions over 30 min to a solution of 2-(tert-butoxy)acetic acid (15.00 g, 113.50 mmol), N,O-dimethylhydroxylamine hydrochloride(16.5 g, 169.15 mmol) and 4-methylmorpholine (18.400 g, 20 mL, 181.91 mmol) in DCM (210 mL) chilled in an ice-water bath (internal temperature 1.5° C.). At the end of the additions, the mixture was stirred 1 h in the ice-water bath, then the reaction mixture was allowed to slowly warm up to ambient temperature overnight. 2 M HCl (90 mL, 180.00 mmol) was added and the aqueous phase was extracted with DCM (210 mL). The combined organic extracts were washed with saturated aqueous NaHCO₃ (150 mL), H₂O (150 mL) and brine (150 mL). The combined organic extracts were dried, filtered and concentrated in vacuo to give 2-(tert-butoxy)-N-methoxy-N-methylacetamide (19.8 g, 100%) as a light brown oil. ESI-MS m/z calc. 175.1208, found 198.2 (M+1)⁺; Retention time: 1.39 minutes.

### Step 2:

ⁿBuLi (4.8 mL, 2 M solution, 9.6 mmol was added to a stirred solution of 2,5-dibromopyridine (1.5 g, 6.332 mmol) in toluene (20 mL) at −78° C. After stirring for 5 min, a solution of 2-(tert-butoxy)-N-methoxy-N-methylacetamide (1.6 g, 9.131 mmol) in toluene (5 mL) was added to the reaction mixture. The mixture was stirred at −78° C. for 45 min. The mixture was quenched by addition of a saturated NH₄Cl solution, extracted with EtOAc (3×50 mL). The combined organic extracts were washed with water (50 mL), brine (1×50 mL), dried (Na₂SO₄), filtered and concentrated in vacuo. Purification by flash chromatography (SiO₂, 5 to 10% EtOAc in hexanes) gave 1-(5-bromopyridin-2-yl)-2-(tert-butoxy)ethan-1-one (850 mg, 49%). ¹H NMR (400 MHz, DMSO-d₆) δ 8.84 (d, J=1.64 Hz, 1H), 8.29 (dd, J=1.68 Hz, J=8.24 Hz, 1H), 7.87 (d, J=8.36, 1H), 4.91 (s, 2H), 1.18 (s, 9H) ppm.

### Step 3:

Sodium borohydride (56 mg, 1.480 mmol) was added to a stirred solution of 1-(5-bromopyridin-2-yl)-2-(tert-butoxy)ethan-1-one (1.2 g, 4.410 mmol) in MeOH (10 mL) at 0° C. and the mixture was stirred at ambient temperature for 1 h. The reaction mixture was concentrated in vacuo. The residue was partitioned between water (10 mL) and EtOAc (50 mL). The aqueous phase was extracted further with EtOAc (2×50 mL). The combined organic extracts were washed with brine (1×50 mL), dried (Na₂SO₄), filtered and concentrated in vacuo. Purification by flash chromatography (SiO₂, 15% EtOAc in hexanes) gave rac-1-(5-bromopyridin-2-yl)-2-(tert-butoxy)ethan-1-ol (750 mg, 59%) as a white solid. ¹H NMR (400 MHz, DMSO-d₆) δ 8.61 (d, J=1.96 Hz, 1H), 8.02 (d, J=2.28 Hz, 5.6 Hz, 1H), 7.47 (d, J=8.4 Hz, 1H), 5.43 (d, J=5.12 Hz, 1H), 4.61 (dd, J=5.44, 11.2 Hz, 1H), 3.59 (dd, J=4.12 Hz, 6.46, 9.2 Hz, 1H), 3.42 (t, J=6.76 Hz, 1H), 1.06 (s, 9H) ppm. ESI-MS m/z calc. 273.0364, found 276.0 (M+1); Retention time: 3.24 minutes.

### Step 4:

DAST (132 mg, 0.819 mmol) was added to a stirred solution of rac-1-(5-bromopyridin-2-yl)-2-(tert-butoxy)ethan-1-ol (150 mg, 0.547 mmol) in DCM (5 mL) at −78° C. At the end of addition, the reaction mixture was stirred at ambient temperature for 1 h. The reaction mixture was quenched by addition of water and extracted with EtOAc (2×40 mL). The combined organic extracts were washed with saturated NaHCO₃ (1×25 mL), brine (1×25 mL), dried (Na₂SO₄), filtered and concentrated in vacuo to give a pale yellow sticky compound. Purification by flash chromatography (SiO₂, 2 to 4% EtOAc in hexanes) gave rac-5-bromo-2-(2-(tert-butoxy)-1-fluoroethyl)pyridine (150 mg, 99%) as a pale yellow oil. ¹H NMR (400 MHz, DMSO-d₆) δ 8.71 (d, J=1.36 Hz, 1H), 8.14 (dd, J=7.84 Hz, 8.32 Hz, 1H), 7.48 (d, J=8.36 Hz, 1H), 5.64-5.51 (m, 1H), 3.81-3.69 (m, 2H), 1.09 (s, 9H) ppm. ESI-MS m/z calc. 275.0321, found 3.6 (M+1)⁺; Retention time: 3.6 minutes.

The following intermediate was made using a method similar to that described in Intermediate BP except that, the conditions used for Step 2 where those described in Intermediate 11 Step 1, using 2-bromo-4-chloropyridine as the starting material in place of 2,5-dibromopyridine:

| Intermediate | | | |
|---|---|---|---|
| No. | Compound Name | LC/MS | NMR (shifts in ppm) |
| BQ | rac-2-(2-(tert-butoxy)-1- | ESI-MS m/z calc. | ¹H NMR (400 MHz, |
| | fluoroethyl)-4-chloropyridine | 231.0826, found 232.0 | DMSO-d₆) δ 8.55 (d, J = |
| | | (M + 1)⁺; Retention time: | 5.2 Hz, 1H), 7.59-7.53 |
| | | 1.94 minutes | (m, 2H), 5.69-5.54 (m, |
| | | | 1H), 3.89-3.71 (m, 2H), |
| | | | 1.09 (s, 9H) ppm. |

### Intermediate BR

### 2-(5-bromo-1-methyl-1H-imidazol-2-yl)ethan-1-ol

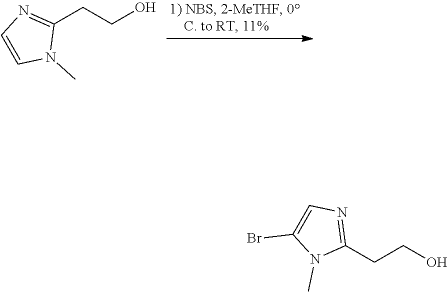

### Step 1:

NBS (673 mg, 3.781 mmol) was added portionwise over 5 min to a stirred solution of 2-(1-methyl-1H-imidazol-2-yl)ethan-1-ol (465 mg, 3.686 mmol) in 2-MeTHF (7 mL) cooled in an acetone-ice bath. The resulting suspension was warmed to ambient temperature and stirred for 18 h. The mixture was partitioned between saturated aqueous NaHCO₃ (30 mL) and EtOAc (40 mL). The organic phase was separated, washed with brine (35 mL), passed through a phase separator cartridge and the filtrate was concentrated in vacuo. Purification by flash chromatography (12 g SiO₂, 0 to 100% EtOAc in hexanes then, 100% 3:1 EtOAc:EtOH) gave 2-(5-bromo-1-methyl-1H-imidazol-2-yl)ethan-1-ol (81 mg, 11%) as a clear oil. H NMR (400 MHz, DMSO-d₆) δ 6.88 (s, 1H), 4.74 (t, J=5.4 Hz, 1H), 3.68 (td, J=6.8, 5.4 Hz, 2H), 3.52 (s, 3H), 2.82 (t, J=6.8 Hz, 2H) ppm.

### Intermediate BS

### tert-butyl rac-(1-(4-aminopyridin-2-yl)-2-methoxy-2-methylpropyl)(methyl)carbamate

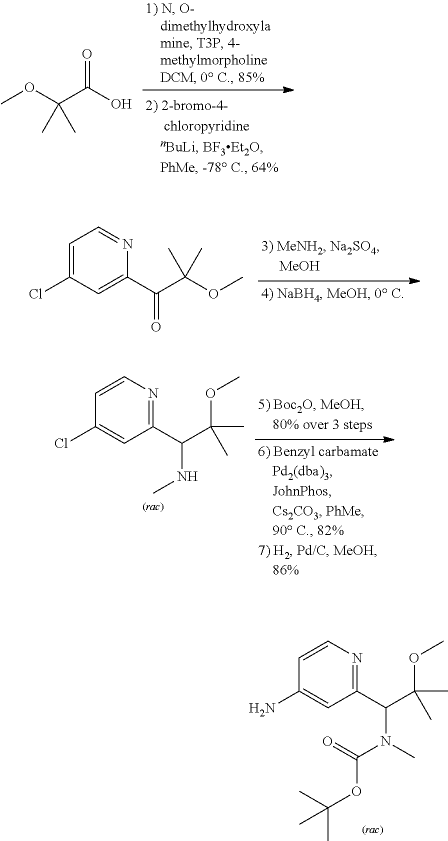

### Step 1:

4-Methylmorpholine (21.436 g, 23.3 mL, 211.93 mmol) was added dropwise to a stirred solution of 2-methoxy-2-methylpropanoic acid (12.5 g, 105.81 mmol) and N,O-dimethylhydroxylamine (12.4 g, 127.12 mmol) in anhydrous DCM (600 mL) at 0° C. under argon. The reaction mixture was stirred for 5 min at 0° C. before adding T3P (81 mL, 50% w/v solution in DCM, 127.29 mmol) dropwise over 5 min. The reaction mixture was then stirred at 0° C. for 2 h. The reaction was warmed up to ambient temperature and diluted with DCM (250 mL) and water (250 mL). The pH of the mixture was adjusted to pH 3-4 by addition of acetic acid. The aqueous solution was separated and extracted with DCM (3×300 mL). The combined organic extracts were washed with saturated NaHCO₃, dried (Na₂SO₄), filtered and concentrated in vacuo to give N,2-dimethoxy-N,2-dimethylpropanamide (15.6 g, 85%) as light yellow oil containing 10% DCM. The product was used in the next step without further purification. ¹H NMR (500 MHz, DMSO-d₆) δ 3.62 (s, 3H), 3.24 (s, 3H), 3.12 (s, 3H), 1.32 (s, 6H) ppm. ESI-MS m/z calc. 161.1052, found 162.7 (M+1)⁺; Retention time: 1.58 minutes.

### Step 2:

ⁿBuLi (30 mL, 2 M in hexanes, 60.0 mmol) was added to the stirred solution of 2-bromo-4-chloropyridine (7.5 g, 38.973 mmol) in toluene (100 mL) at −78° C. After stirring for 10 min, a solution of N,2-dimethoxy-N,2-dimethylpropanamide (7 g, 43.425 mmol) in toluene (20 mL) was added over 2 min and the reaction mixture was stirred for 5 min at −78° C. BF₃.Et₂O (2.76 g, 2.4 mL, 19.446 mmol) was added and the reaction mixture was stirred for 10 min at −78° C. The mixture was quenched by addition of a saturated NH₄Cl solution. The aqueous phase was separated and extracted with ethyl acetate (1×200 mL). The combined organic extracts were washed with brine, dried (Na₂SO₄), filtered and concentrated in vacuo. Purification by flash chromatography (SiO₂, 10 to 20% EtOAc in hexanes) gave 1-(4-chloropyridin-2-yl)-2-methoxy-2-methylpropan-1-one (5.8 g, 64%) as yellow oil. ¹H NMR (400 MHz, Chloroform-d) δ 8.61 (d, J=5.2 Hz, 1H), 8.03 (d, J=2 Hz, 1H), 7.46-7.44 (m, 1H), 3.26 (s, 3H), 1.68 (s, 6H) ppm. ESI-MS m/z calc. 213.0557, found 214.0 (M+1)⁺; Retention time: 1.78 minutes.

### Step 3:

Methanamine (6 mL, 9.8 M in MeOH, 58.8 mmol) and sodium sulfate (6 g, 42.241 mmol) were successively added to an oven dried microwave vial (25 mL) containing 1-(4-chloropyridin-2-yl)-2-methoxy-2-methylpropan-1-one (1 g, 4.680 mmol). The vial was sealed and heated in an oil bath at 80° C. for 4.5 h. The mixture was filtered to give 1-(4-chloropyridin-2-yl)-2-methoxy-N,2-dimethylpropan-1-imine (1.061 g) as a single geometric isomer (E/Z geometry not characterized), which was used without further purification in the next step. ¹H NMR (500 MHz, Chloroform-d) δ 8.57 (dd, J=5.4, 0.7 Hz, 1H), 7.28 (dd, J=5.4, 2.0 Hz, 1H), 7.14 (dd, J=2.0, 0.7 Hz, 1H), 3.25 (s, 3H), 3.01 (s, 3H), 1.39 (s, 6H) ppm.

### Step 4:

NaBH₄ (177 mg, 4.679 mmol) was added to the crude 1-(4-chloropyridin-2-yl)-2-methoxy-N,2-dimethylpropan-1-imine (6 mL, 0.78 M solution, 4.680 mmol) at ambient temperature. The mixture was diluted with methanol (15 mL) and stirred for 10 min at ambient temperature. The resulting rac-1-(4-chloropyridin-2-yl)-2-methoxy-N,2-dimethylpropan-1-amine (107 mg) was used directly in the next step without purification. ¹H NMR (500 MHz, Chloroform-d) δ 8.43 (dd, J=5.3, 0.6 Hz, 1H), 7.53 (d, J=2.1 Hz, 1H), 7.18 (dd, J=5.3, 2.1 Hz, 1H), 3.70 (d, J=4.3 Hz, 1H), 3.25 (s, 3H), 2.24 (d, J=6.4 Hz, 3H), 1.10 (d, J=1.5 Hz, 6H) ppm; NH amine not observed.

### Step 5:

Boc₂O (3.07 g, 14.067 mmol) was added to the crude solution of rac-1-(4-chloropyridin-2-yl)-2-methoxy-N,2-dimethylpropan-1-amine in MeOH (21 mL, 0.223 M solution, 4.6830 mmol) and the mixture stirred for 14 h at ambient temperature. Additional Boc₂0 (1.53 g, 7.010 mmol) was added and the stirring was continued for a further 30 min. The reaction mixture was concentrated in vacuo. The residue was dissolved in EtOAc (50 mL), washed with saturated NH₄Cl (2 mL) and water (1 mL), then NaHCO₃ (10 mL), dried (Na₂SO₄), filtered and concentrated in vacuo. Purification by flash chromatography (120 g SiO₂, 0 to 30% EtOAc in hexanes containing 1% Et₃N) gave tert-butyl rac-(1-(4-chloropyridin-2-yl)-2-methoxy-2-methylpropyl)(methyl)carbamate (1.251 g, 80% over 3 steps) as a colourless oil. ¹H NMR (500 MHz, Chloroform-d) δ 8.48 (d, J=5.2 Hz, 1H), 7.68 (s, 1H), 7.18 (dd, J=5.3, 2.0 Hz, 1H), 5.60-5.11 (m, 1H), 3.26 (s, 3H), 2.91 (s, 3H), 1.47 (s, 9H), 1.32 (s, 3H), 1.23 (s, 3H) ppm. ESI-MS m/z calc. 328.1554, found 329.4 (M+1)⁺; Retention time: 4.64 minutes.

### Step 6:

Dry, degassed toluene (20 mL) (pre-sparged with Argon for 1 h) was added to a flask containing benzyl carbamate (1.75 g, 11.577 mmol), Pd₂(dba)₃ (173 mg, 0.189 mmol), JohnPhos (117 mg, 0.392 mmol), tert-butyl rac-(1-(4-chloropyridin-2-yl)-2-methoxy-2-methylpropyl)(methyl)carbamate (1.25 g, 3.763 mmol) and Cs₂CO₃ (3.35 g, 10.282 mmol). The mixture was heated at 90° C. in an oil bath for 4 h. The reaction was cooled to ambient temperature, concentrated in vacuo, diluted with EtOAc (50 mL) and filtered through a pad of Celite, rinsing with EtOAc (50 mL). The filtrates were concentrated in vacuo. Purification by flash chromatography (120 g SiO₂, 0 to 50% EtOAc in hexanes containing 1% Et₃N) gave tert-butyl rac-(1-(4-(((benzyloxy)carbonyl)amino)pyridin-2-yl)-2-methoxy-2-methylpropyl)(methyl)carbamate (1.72 g, 82%). ESI-MS m/z calc. 443.242, found 444.6 (M+1)⁺; Retention time: 5.03 minutes.

### Step 7:

MeOH (15.75 mL) was added under N₂ to a flask containing tert-butyl rac-(1-(4-(((benzyloxy)carbonyl)amino)pyridin-2-yl)-2-methoxy-2-methylpropyl)(methyl)carbamate (1.05 g, 2.367 mmol) and Pd/C (wet 50% H₂O) (252 mg, 10% w/w, 0.237 mmol). The flask was evacuated and backfilled with hydrogen gas (3 x), then stirred under a hydrogen atmosphere (balloon) at ambient temperature for 30 min. The hydrogen balloon was removed and the atmosphere was replaced with inert gas (N₂). The reaction mixture was filtered through a pad of Celite and the filtrates were concentrated in vacuo to give tert-butyl rac-(1-(4-aminopyridin-2-yl)-2-methoxy-2-methylpropyl)(methyl)carbamate (673 mg, 86%) as an off-white foam. ¹H NMR (500 MHz, Chloroform-d) δ 8.20 (d, J=5.6 Hz, 1H), 6.87 (d, J=2.3 Hz, 1H), 6.41 (dd, J=5.6, 2.3 Hz, 1H), 5.45-4.99 (m, 1H), 4.07 (s, 2H), 3.24 (s, 3H), 2.93 (s, 3H), 1.47 (s, 9H), 1.31 (s, 3H), 1.26 (s, 3H) ppm. ESI-MS m/z calc. 309.2052, found 310.3 (M+1)⁺; Retention time: 1.81 minutes.

### Intermediate BT

### tert-butyl rac-(1-(4-amino-5-fluoropyridin-2-yl)-2-methoxy-2-methylpropyl)carbamate

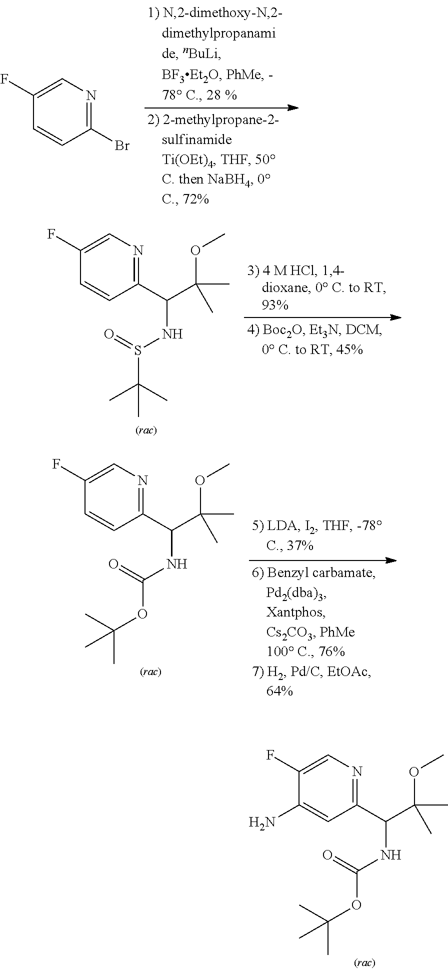 [SMILES: Fc1ccc(Br)nc1 ; COC(C)(C)C(NS(=O)C(C)(C)C)c1ccc(F)cn1 ; COC(C)(C)C(NC(=O)OC(C)(C)C)c1ccc(F)cn1 ; COC(C)(C)C(NC(=O)OC(C)(C)C)c1cc(N)c(F)cn1]

### Step 1:

ⁿBuLi (45 mL, 2 M in hexanes, 90.0 mmol) was added to a stirred solution of 2-bromo-5-fluoropyridine (10 g, 56.823 mmol) in toluene (100 mL) at −78° C. and the reaction mixture was stirred for 10 min. A solution of N,2-dimethoxy-N,2-dimethylpropanamide (10 g, 62.035 mmol) in toluene (40 mL) and BF₃.Et₂O (4.0250 g, 3.5 mL, 28.359 mmol) were successively added to the reaction mixture which was stirred at −78° C. for 10 min. The mixture was quenched by addition of a saturated aqueous ammonium chloride solution (100 mL). The aqueous phase was extracted with ethyl acetate (2×100 mL). The combined organic extracts were washed with brine (100 mL), dried (Na₂SO₄), filtered and concentrated in vacuo. Purification by flash chromatography (SiO₂, 5% EtOAc in hexanes) gave 1-(5-fluoropyridin-2-yl)-2-methoxy-2-methylpropan-1-one (3.12 g, 28%) as a brown oil. ¹H NMR (400 MHz, DMSO-d₆) δ 8.69 (d, J=2.8 Hz, 1H), 8.05-8.02 (m, 1H), 7.92-7.87 (m, 1H), 3.12 (s, 3H), 1.57 (s. 6H) ppm. ESI-MS m/z calc. 197.0852, found 198.1 (M+1)⁺; Retention time: 3.03 minutes.

### Step 2:

2-Methylpropane-2-sulfinamide (920 mg, 7.5907 mmol) and Ti(OEt)₄ (1.741 g, 1.6 mL, 7.631 mmol) were successively added to a stirred solution of 1-(5-fluoropyridin-2-yl)-2-methoxy-2-methylpropan-1-one (1 g, 5.071 mmol) in THF (10 mL) and the mixture was heated at 50° C. for 5 h. The mixture was cooled to 0° C. before adding sodium borohydride (670 mg, 17.710 mmol) and stirring for a further 1 h at 0° C. The mixture was quenched by addition of a saturated aqueous NaHCO₃ solution (20 mL), stirred for 10 min and filtered through a pad of Celite. The filtrates were concentrated in vacuo to give rac-N-(1-(5-fluoropyridin-2-yl)-2-methoxy-2-methylpropyl)-2-methylpropane-2-sulfinamide (1.1 g, 72%) as a brown oil. ESI-MS m/z calc. 302.1464, found 303.1 (M+1)⁺; Retention time: 3.14 minutes.

### Step 3:

HCl (10 mL, 4 M in 1,4-dioxane, 40.0 mmol) was slowly added at 0° C. to a stirred solution of rac-N-(1-(5-fluoropyridin-2-yl)-2-methoxy-2-methylpropyl)-2-methylpropane-2-sulfinamide (1 g, 3.307 mmol) in 1,4-dioxane (5 mL). The reaction mixture was stirred at ambient temperature for 2 h. The mixture was concentrated in vacuo to give rac-1-(5-fluoropyridin-2-yl)-2-methoxy-2-methylpropan-1-amine (Hydrochloride salt) (720 mg, 93%) as a brown oil. ESI-MS m/z calc. 198.1168, found 199.0 (M+1)⁺; Retention time: 2.03 minutes.

### Step 4:

Boc₂O (3.8 g, 4 mL, 17.411 mmol) was added to a stirred solution of rac-1-(5-fluoropyridin-2-yl)-2-methoxy-2-methylpropan-1-amine (Hydrochloride salt) (3.8 g, 16.191 mmol) and triethylamine (3.63 g, 5 mL, 35.873 mmol) in DCM (50 mL) at 0° C. and the mixture was stirred at ambient temperature for 3 h. The reaction mixture was concentrated in vacuo. Purification by flash chromatography (SiO₂, 10% EtOAc in hexanes) gave tert-butyl rac-(1-(5-fluoropyridin-2-yl)-2-methoxy-2-methylpropyl)carbamate (2.16 g, 45%) as a white solid. ¹H NMR (400 MHz, DMSO-d₆) δ 8.46 (d, J=2.9 Hz, 1H), 7.69-7.64 (m, 1H), 7.52-7.48 (m, 1H), 6.97 (d, J=9.4 Hz, 1H), 4.77 (d, J=9.7 Hz, 1H), 3.11 (s, 3H), 1.36 (s, 9H), 1.03 (s, 6H) ppm. ESI-MS m/z calc. 298.1693, found 242.9 (M−55)⁺; Retention time: 3.4 minutes.

### Step 5:

LDA (2.5 mL, 2 M solution in THF, heptane and ethylbenzene, 5.0 mmol) was slowly added at −78° C. to a stirred solution of tert-butyl rac-(1-(5-fluoropyridin-2-yl)-2-methoxy-2-methylpropyl) carbamate (1 g, 3.352 mmol) in THF (10 mL) and the reaction mixture was stirred for 1 h. A solution of iodine (1.1 g, 4.3340 mmol) in THF (5 mL) was slowly added at −78° C. and the reaction mixture was stirred for a further 30 min. The mixture was quenched by addition of a saturated aqueous ammonium chloride solution (10 mL), diluted with water (10 mL) and slowly allowed to warm to ambient temperature. The mixture was extracted with ethyl acetate (2×30 mL). The combined organic layers were washed with brine (20 mL), dried (Na₂SO₄), filtered and concentrated in vacuo. Purification by flash chromatography (SiO₂, 5% EtOAc in hexanes) gave tert-butyl rac-(1-(5-fluoro-4-iodopyridin-2-yl)-2-methoxy-2-methylpropyl)carbamate (530 mg, 37%) as a white solid. ¹H NMR (400 MHz, DMSO-d₆) δ 8.38 (s, 1H), 7.98 (s, 1H), 7.07 (d, J=8.9 Hz, 1H), 4.73 (d, J=9 Hz, 1H), 3.11 (s, 3H), 1.36 (s, 9H), 1.04 (d, J=10.4 Hz, 6H) ppm. ESI-MS m/z calc. 424.0659, found 369.23 (M−55)⁺; Retention time: 1.89 minutes.

### Step 6:

Benzyl carbamate (35 mg, 0.232 mmol) and cesium carbonate (150 mg, 0.460 mmol) were successively added to a stirred solution of tert-butyl rac-(1-(5-fluoro-4-iodopyridin-2-yl)-2-methoxy-2-methylpropyl)carbamate (100 mg, 0.236 mmol) in toluene (2 mL). The reaction mixture was purged with argon. Pd₂(dba)₃ (5 mg, 0.006 mmol) and Xantphos (5 mg, 0.009 mmol) were successively added and the reaction mixture was heated at 100° C. for 3 h. The mixture was cooled to ambient temperature, filtered through a pad of Celite and concentrated in vacuo to give tert-butyl rac-(1-(4-(((benzyloxy)carbonyl)amino)-5-fluoropyridin-2-yl)-2-methoxy-2-methylpropyl)carbamate (80 mg, 76%) as a brown solid. ESI-MS m/z calc. 447.2169, found 448.28 (M+1)⁺; Retention time: 1.93 minutes.

### Step 7:

Pd/C (100 mg, 10% w/w, 0.094 mmol) was added to a stirred solution of tert-butyl rac-(1-(4-(((benzyloxy)carbonyl)amino)-5-fluoropyridin-2-yl)-2-methoxy-2-methylpropyl)carbamate (400 mg, 0.894 mmol) in ethyl acetate (5 mL). The mixture was degassed with argon and stirred at ambient temperature under a hydrogen atmosphere (balloon) for 5 h. The mixture was filtered through a pad of Celite and the filtrates were concentrated in vacuo to give tert-butyl rac-(1-(4-amino-5-fluoropyridin-2-yl)-2-methoxy-2-methylpropyl)carbamate (180 mg, 64%) as a brown solid. ESI-MS m/z calc. 313.1802, found 258.22 (M−55)⁺; Retention time: 1.36 minutes.

### Intermediate BU

### tert-butyl rac-(2-(4-aminopyridin-2-yl)-1-methoxypropan-2-yl)carbamate

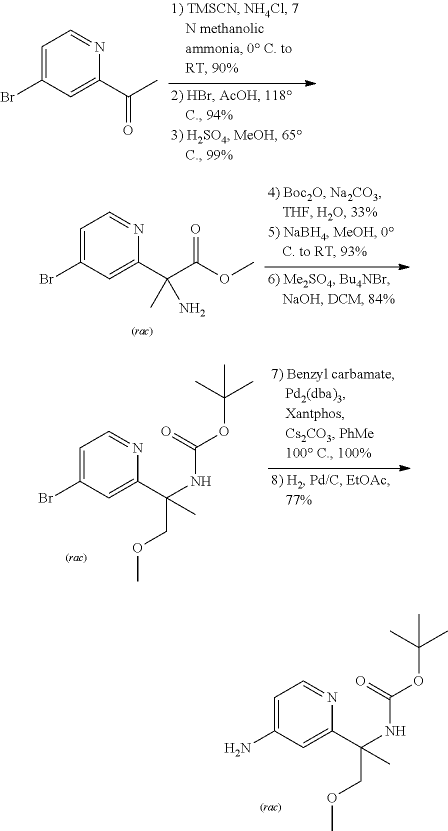

### Step 1:

Trimethylsilyl cyanide (15.860 g, 20 mL, 159.87 mmol) was added at 0° C. to a stirred solution of 1-(4-bromopyridin-2-yl)ethan-1-one (10 g, 49.992 mmol) and ammonium chloride (8.2 g, 5.3595 mL, 153.30 mmol) in 7 N methanolic ammonia (330 mL) and the mixture was stirred at ambient temperature for 24 h. The solvent was evaporated in vacuo and the residue was taken up in DCM (200 mL). The solid was filtered and the filtrate were concentrated in vacuo to give rac-2-amino-2-(4-bromopyridin-2-yl)propanenitrile (10.2 g, 90%). ESI-MS m/z calc. 224.9902, found 226.2 (M+1)⁺; Retention time: 2.62 minutes.

### Step 2:

HBr (149.00 g, 100 mL, 33% w/v in AcOH, 1.842 mol) was added to rac-2-amino-2-(4-bromopyridin-2-yl)propanenitrile (10 g, 44.233 mmol) and the mixture was heated to 118° C. for 12 h. The reaction mixture was cooled to ambient temperature. Ethyl acetate (60 mL) was added and the formed precipitate was filtered off and washed with ethyl acetate (90 mL). The solid was dried to give rac-2-amino-2-(4-bromopyridin-2-yl)propanamide (10.2 g, 94%). ESI-MS m/z calc. 243.0007, found 245.9 (M+1)⁺; Retention time: 1.59 minutes.

### Step 3:

H₂SO₄ (7.360 g, 4 mL, 75.041 mmol) was added to a stirred solution of rac-2-amino-2-(4-bromopyridin-2-yl)propanamide (8 g, 32.775 mmol) in MeOH (25 mL). The reaction mixture was heated at 65° C. for 24 h. The reaction mixture was concentrated in vacuo to give methyl rac-2-amino-2-(4-bromopyridin-2-yl)propanoate (8.4 g, 99%) as a brown oil. ¹H NMR (400 MHz, DMSO-d₆) δ 8.36 (d, J=5.24 Hz, 1H), 7.94 (d, J=1.24 Hz, 1H), 7.57-7.55 (m, 1H), 3.57 (s, 3H), 2.55 (s, 2H), 1.5 (s, 3H) ppm.

### Step 4:

Sodium carbonate (13.5 g, 127.37 mmol) was added to a stirred solution of methyl rac-2-amino-2-(4-bromopyridin-2-yl)propanoate (7 g, 27.017 mmol) in THF (42 mL) and H₂O (18 mL) and the pH was adjusted to 9-10. Boc₂0 (14.250 g, 15 mL, 65.293 mmol) was added and the mixture was stirred at ambient temperature for 24 h. The reaction mixture was extracted with ethyl acetate (300 mL). The organic phase was separated and concentrated in vacuo. Purification by flash chromatography (SiO₂, 10% EtOAc in hexanes) gave methyl rac-2-(4-bromopyridin-2-yl)-2-((tert-butoxycarbonyl)amino)propanoate (3.2 g, 33%) as a light yellow solid. ¹H NMR (400 MHz, DMSO-d₆) δ 8.43 (d, J=5.32 Hz, 1H), 7.73 (d, J=1.6 Hz, 1H), 7.66-7.64 (m, 1H), 7.49 (br s, 1H), 3.59 (s, 3H), 1.75 (s, 3H), 1.37 (s, 9H) ppm.

### Step 5:

Sodium borohydride (1.6 g, 42.292 mmol) was added at 0° C. to a stirred solution of methyl rac-2-(4-bromopyridin-2-yl)-2-((tert-butoxycarbonyl)amino)propanoate (3 g, 8.352 mmol) in methanol (30 mL) and the reaction mixture was stirred at ambient temperature for 12 h. The mixture was extracted with ethyl acetate (400 mL). The organic phase was separated and concentrated in vacuo. Purification by flash chromatography (SiO₂, 50% EtOAc in hexanes) gave tert-butyl rac-(2-(4-bromopyridin-2-yl)-1-hydroxypropan-2-yl)carbamate (2.6 g, 93%) as a colourless oil. ESI-MS m/z calc. 330.0579, found 275.0 (M−56)⁺; Retention time: 1.64 minutes.

### Step 6:

NaOH (16 mL, 1 M aqueous solution, 16. 0 mmol) and Bu₄NBr (5 g, 15.510 mmol) was added to a stirred solution of tert-butyl rac-(2-(4-bromopyridin-2-yl)-1-hydroxypropan-2-yl)carbamate (2.5 g, 7.5482 mmol) in DCM (25 mL) and the mixture was stirred for 15 min. Dimethyl sulphate (2.261 g, 1.7 mL, 17.926 mmol) was added and the reaction mixture was stirred for 16 h at ambient temperature. The mixture was diluted with water (500 mL) and extracted with DCM (700 mL). The organic layer was dried (MgSO₄), filtered and concentrated in vacuo. Purification by flash chromatography (SiO₂, 5 to 10% EtOAc in hexanes) gave tert-butyl rac-(2-(4-bromopyridin-2-yl)-1-methoxypropan-2-yl)carbamate (2.2 g, 84%) as a colourless oil. ¹H NMR (400 MHz, DMSO-d₆) δ 8.38 (d, J=5.24 Hz, 1H), 7.56 (d, J=1.36 Hz, 1H), 7.53-7.51 (m, 1H), 7.03 (br s, 1H), 3.68-3.64 (m, 2H), 3.20 (s, 3H), 1.50 (s, 3H), 1.34 (s, 9H) ppm.

### Step 7:

Benzyl carbamate (895 mg, 5.921 mmol) and cesium carbonate (3.8 g, 11.663 mmol) were successively added to a stirred solution of tert-butyl rac-(2-(4-bromopyridin-2-yl)-1-methoxypropan-2-yl)carbamate (2 g, 5.793 mmol) in toluene (40 mL) and the mixture was purged with argon. Pd₂(dba)₃ (105 mg, 0.115 mmol) and xantphos (100 mg, 0.173 mmol) were added and the reaction mixture was heated at 100° C. for 3 h. The mixture was cooled to ambient temperature, filtered through a pad of Celite and concentrated in vacuo. Purification by flash chromatography (SiO₂, 7 to 10% EtOAc in hexanes) gave tert-butyl rac-(2-(4-(((benzyloxy)carbonyl)amino)pyridin-2-yl)-1-methoxypropan-2-yl)carbamate (2.5 g, 100%) as an off-white solid. ¹H NMR (400 MHz, DMSO-d₆) δ 10.19 (s, 1H), 8.27 (d, J=5.48 Hz, 1H), 7.58 (s, 1H), 7.44-7.28 (m, 6H), 6.89 (br s, 1H), 5.17 (s, 2H), 3.69-3.63 (m, 2H), 3.19 (s, 3H), 1.46 (s, 3H), 1.35 (s, 9H) ppm. ESI-MS m/z calc. 415.2107, found 416.2 (M+1)⁺; Retention time: 3.46 minutes.

### Step 8:

Pd/C (750 mg, 7.048 mmol) was added to a stirred solution of tert-butyl rac-(2-(4-(((benzyloxy)carbonyl)amino)pyridin-2-yl)-1-methoxypropan-2-yl)carbamate (2.9 g, 6.980 mmol) in ethyl acetate (40 mL). The reaction mixture was degassed with Argon and stirred at ambient temperature under a hydrogen atmosphere (balloon) for 12 h. The mixture was filtered through a pad of Celite and concentrated in vacuo to give tert-butyl rac-(2-(4-aminopyridin-2-yl)-1-methoxypropan-2-yl)carbamate (1.51 g, 77%) as a colourless sticky oil. ¹H NMR (400 MHz, DMSO-d₆) δ 7.86 (d, J=5.52 Hz, 1H), 6.88 (br s, 1H), 6.5 (d, J=1.68 Hz, 1H), 6.32-6.30 (m, 1H), 5.91 (s, 2H), 3.69-3.67 (m, 1H), 3.60-3.58 (m, 1H), 3.15 (s, 3H), 1.40 (s, 3H), 1.34 (s, 9H) ppm. ESI-MS m/z calc. 281.1739, found 282.3 (M+1)⁺; Retention time: 1.25 minutes.

The following intermediate was made using a method similar to that described in Intermediate BU except that, in Step 1, 1-(5-fluoropyridin-2-yl)ethan-1-one was used as starting material in place of 1-(4-bromopyridin-2-yl)ethan-1-one. Step 4 was omitted. In Step 5, the reduction was carried out with LiBH₄ in place of NaBH₄ in THF rather than MeOH as the solvent. In Step 6, THF was used as the solvent in place of DCM. Steps 7 and 8 were omitted. The product of Step 6 was further reacted with iodine using the conditions described in Intermediate 20 Step 5. A final amination Step was carried out at 90° C. in the presence of a catalytic amount of CuI, an excess of ammonium hydroxide, K₂CO₃ as the base and DMSO as the solvent, conditions well known in the art:

| Intermediate | | | |
|---|---|---|---|
| No. | Compound Name | LC/MS | NMR (shifts in ppm) |
| BV | tert-butyl rac-(2-(4-amino-5- | ESI-MS m/z calc. | ¹H NMR (400 MHz, |
| | fluoropyridin-2-yl)-1- | 299.1645, found 300.0 | DMSO-d₆) δ 7.96 (d, J = |
| | methoxypropan-2-yl)carbamate | (M + 1)⁺; Retention time: | 2.9 Hz, 1H), 6.80 (br s, |
| | | 1.28 minutes | 1H), 6.74 (d, J = 7.4 Hz, |
| | | | 1H), 6.09 (s, 2H), 3.63 (q, |
| | | | J = 6.2 Hz, 2H), 3.19 (s, |
| | | | 3H), 1.42 (s, 3H), 1.23 (s, |
| | | | 9H) ppm. |

### Intermediate BW

### tert-butyl rac-(2-(4-aminopyridin-2-yl)-1-((tert-butyldimethylsilyl)oxy)propan-2-yl)carbamate

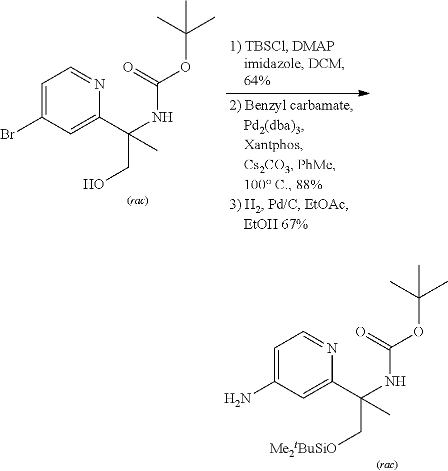

### Step 1:

DMAP (40 mg, 0.3274 mmol) was added to a stirred solution of tert-butyl rac-(2-(4-bromopyridin-2-yl)-1-hydroxypropan-2-yl)carbamate (500 mg, 1.510 mmol) (Product of Intermediate 21, Step 5), imidazole (310 mg, 4.554 mmol) and TBSCl (1.2 g, 7.962 mmol) in DCM (10 mL) and the mixture was stirred at ambient temperature for 16 h. The reaction mixture was quenched by addition of water. The aqueous phase was separated and extracted with DCM (60 mL). The combined organic extracts were dried (MgSO₄), filtered and concentrated in vacuo. Purification by flash chromatography (SiO₂, 7% EtOAc in hexanes) gave tert-butyl rac-(2-(4-bromopyridin-2-yl)-1-((tert-butyldimethylsilyl)oxy)propan-2-yl)carbamate (430 mg, 64%) as a colourless oil. ESI-MS m/z calc. 444.1444, found 364.0 (M−99)⁺; Retention time: 2.89 minutes.

### Step 2:

Benzyl carbamate (140 mg, 0.9262 mmol) and cesium carbonate (600 mg, 1.8415 mmol) were successively added to a stirred solution of tert-butyl rac-(2-(4-bromopyridin-2-yl)-1-((tert-butyldimethylsilyl)oxy)propan-2-yl)carbamate (400 mg, 0.898 mmol) in toluene (8 mL) and the mixture was purged with Argon. Pd₂(dba)₃ (17 mg, 0.019 mmol) and xantphos (16 mg, 0.028 mmol) were successively added and the reaction mixture was heated at 100° C. for 3 h. The mixture was cooled to ambient temperature, filtered through a pad of Celite and concentrated in vacuo. Purification by flash chromatography (SiO₂, 10% EtOAc in hexanes) gave tert-butyl rac-(2-(4-(((benzyloxy)carbonyl)amino)pyridin-2-yl)-1-((tert-butyldimethylsilyl)oxy)propan-2-yl)carbamate (420 mg, 88%) as an off-white solid. ESI-MS m/z calc. 515.2815, found 516.0 (M+1)⁺; Retention time: 2.65 minutes.

### Step 3:

Pd/C (83 mg, 0.078 mmol) was added to a stirred solution of tert-butyl rac-(2-(4-(((benzyloxy)carbonyl)amino)pyridin-2-yl)-1-((tert-butyldimethylsilyl)oxy)propan-2-yl)carbamate (400 mg, 0.776 mmol) in a mixture of ethyl acetate (5 mL) and EtOH (5 mL). The reaction mixture was degassed with Argon and stirred at ambient temperature under a hydrogen atmosphere (balloon) for 12 h. The mixture was filtered through a pad of Celite and concentrated in vacuo to give tert-butyl rac-(2-(4-aminopyridin-2-yl)-1-((tert-butyldimethylsilyl)oxy)propan-2-yl)carbamate (210 mg, 67%) as a white solid. ¹H NMR (400 MHz, DMSO-d₆) δ 7.88 (d, J=5.48 Hz, 1H), 6.85 (br s, 1H), 6.52 (s, 1H), 6.33 (d, J=3.72 Hz, 1H), 5.92 (br s, 2H), 4.04-3.86 (m, 2H), 1.41 (s, 3H), 1.37 (s, 9H), 0.78 (s, 9H), −0.04-−0.12 (m, 6H) ppm. ESI-MS m/z calc. 381.2448, found 381.8 (M+1)⁺; Retention time: 1.74 minutes.

The following intermediate was made using a method similar to that described in Intermediate BW except that an iodination Step, using the conditions described in Intermediate 20 Step 5, was introduced between Step 1 and Step 2. The starting material of Step 1 was prepared using a method similar to that described in Intermediate 21 except that in Step 1, 1-(5-fluoropyridin-2-yl)ethan-1-one was used as starting material in place of 1-(4-bromopyridin-2-yl)ethan-1-one. Step 4 was omitted. In Step 5, the reduction was carried out with LiBH₄ in place of NaBH₄ in THF rather than MeOH as the solvent. Steps 6 to 8 were omitted:

| Intermediate | | | |
|---|---|---|---|
| No. | Compound Name | LC/MS | NMR (shifts in ppm) |
| BX | tert-butyl rac-(2-(4-amino-5- | ESI-MS m/z calc. | ¹H NMR (400 MHz, |
| | fluoropyridin-2-yl)-1-((tert- | 399.2353, found 400.0 | DMSO-d₆) δ 7.96 (d, J = |
| | butyldimethylsilyl)oxy)propan- | (M + 1)⁺; Retention time: | 2.8 Hz, 1H), 6.74-6.72 |
| | 2-yl)carbamate | 1.06 minutes | (m, 2H), 6.08 (s, 2H), |
| | | | 3.88 (s, 2H), 1.38 (s, 3H), |
| | | | 1.35 (s, 9H), 0.79 (s, |
| | | | 9H), −0.07-−0.09 (m, |
| | | | 6H) ppm. |

### Intermediate BY

### 1-((2-(trimethylsilyl)ethoxy)methyl)-1H-1,2,3-triazol-4-amine

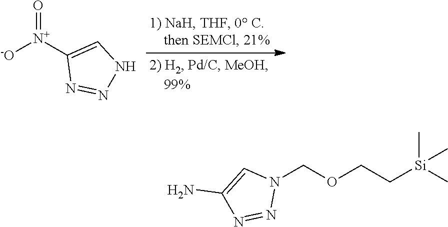

### Step 1:

Sodium hydride (2.279 g of 60% w/w, 56.99 mmol) was added portionwise over 30 min to a stirred solution of 4-nitro-1H-1,2,3-triazole (5 g, 43.84 mmol) in THF (230 mL) cooled in an ice bath. The reaction mixture was stirred at 0° C. for 30 min then, SEMCl (7.674 g, 8.146 mL, 46.03 mmol) was added. The ice bath was removed and the mixture was stirred at ambient temperature for 15 h. The mixture was cooled in an ice bath and was carefully quenched by addition of water. The mixture was extracted with EtOAc (3 x). The combined organic extracts were washed with water, brine, dried (MgSO₄), filtered and concentrated in vacuo to give a brown oil. Purification by flash chromatography (SiO₂, 5% EtOAc in hexanes) gave 4-nitro-1-((2-(trimethylsilyl)ethoxy)methyl)-1H-1,2,3-triazole (6.16 g, 21%) as a pale yellow oil and as the only regioisomer isolated (lower spot by TLC, 5% EtOAc in hexanes). ¹H NMR (500 MHz, DMSO-d₆) δ 8.83 (s, 1H), 5.86 (s, 2H), 3.71 (t, J=10 Hz, 2H), 0.91 (t, J=10 Hz, 2H), 0.00 (s, 9H) ppm.

### Step 2:

A mixture of 4-nitro-1-((2-(trimethylsilyl)ethoxy)methyl)-1H-1,2,3-triazole (1.2 g, 4.912 mmol) and Pd on C (wet, Degussa) (522.7 mg, 10% w/w, 0.491 mmol) in MeOH (21 mL) was stirred under a hydrogen atmosphere (balloon) for 18 h. The reaction mixture was filtered through a pad of Celite, washing with MeOH. The mother liquors were concentrated in vacuo to give 1-((2-(trimethylsilyl)ethoxy)methyl)-1H-1,2,3-triazol-4-amine (1.04 g, 99%) as a sand colour oil, which solidified on standing. ¹H NMR (500 MHz, DMSO-d₆) δ 7.22 (s, 1H), 5.52 (s, 2H), 4.80 (s, 2H), 3.55 (t, J=10 Hz, 2H), 0.88 (t, J=10 Hz, 2H), 0.00 (s, 9H) ppm.

### Intermediate BZ

### rac-2-(2-((tert-butyldimethylsilyl)oxy)-1-(dimethylamino)ethyl)pyridin-4-amine

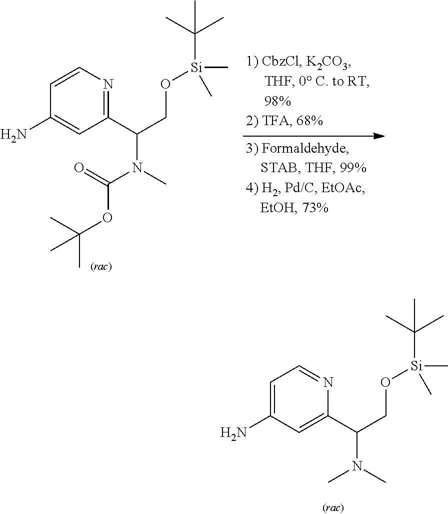 [SMILES: CN(C(=O)OC(C)(C)C)C(CO[Si](C)(C)C(C)(C)C)c1cc(N)ccn1 ; CN(C)C(CO[Si](C)(C)C(C)(C)C)c1cc(N)ccn1]

### Step 1:

Benzyl carbamate (330 μL, 2.312 mmol) was added dropwise to a stirred solution of tert-butyl rac-(1-(4-aminopyridin-2-yl)-2-((tert-butyldimethylsilyl)oxy)ethyl)(methyl)carbamate (570 mg, 1.494 mmol) (Intermediate T) and 2 M K₂CO₃ (1.7 mL, 3.4 mmol) in THF (20 mL) at 0° C. The reaction mixture was warmed to ambient temperature and stirred for 150 min. The mixture was diluted with water (100 mL) and extracted with EtOAc (50 mL). The aqueous layer was separated and extracted further with EtOAc (50 mL). The combined organic extracts were dried (MgSO₄), filtered and concentrated in vacuo. Purification by flash chromatography (SiO₂, 0 to 40% EtOAc in heptane) gave tert-butyl rac-(1-(4-(((benzyloxy)carbonyl)amino)pyridin-2-yl)-2-((tert-butyldimethylsilyl)oxy)ethyl)(methyl)carbamate (782 mg, 98%). ESI-MS m/z calc. 515.28156, found 516.8 (M+1); Retention time: 1.27 minutes.

### Step 2:

A mixture of tert-butyl rac-(1-(4-(((benzyloxy)carbonyl)amino)pyridin-2-yl)-2-((tert-butyldimethylsilyl)oxy)ethyl)(methyl)carbamate (530 mg, 1.028 mmol) and TFA (3 mL) was stirred at ambient temperature for 5 min. The mixture was concentrated in vacuo. The residue was dissolved in MeOH (3 mL) and loaded onto an SCX cartridge (5 g). The cartridge was washed with MeOH (20 mL) and the compound was released by washing the cartridge with 2 M methanolic ammonia (20 mL). The ammonia wash was concentrated in vacuo. Purification by flash chromatography (24 g SiO₂, 0 to 100% (3:1) EtOH/EtOAc containing 2% NH₄OH in heptane) gave benzyl rac-(2-(2-((tert-butyldimethylsilyl)oxy)-1-(methylamino)ethyl)pyridin-4-yl)carbamate (290 mg, 68%) as a white solid. ESI-MS m/z calc. 415.22913, found 416.4 (M+1)⁺; 414.7 (M−1)⁻; Retention time: 1.02 minutes.

### Step 3:

Formaldehyde (2.244 mL, 37% aqueous solution, 81.46 mmol) and sodium triacetoxyborohydride (336.2 mg, 1.594 mmol) were successively added to a solution of benzyl rac-(2-(2-((tert-butyldimethylsilyl)oxy)-1-(methylamino)ethyl)pyridin-4-yl)carbamate (290 mg, 0.698 mmol) in THF (5 mL) and the reaction mixture was stirred at ambient temperature for 3 h. The mixture was concentrated under a stream of nitrogen. EtOAc (10 mL) was added and the mixture was washed with a saturated aqueous NaHCO₃ solution (10 mL) and brine (10 mL). The organic phase was dried by passing through a phase separation cartridge. The liquors were concentrated in vacuo to give benzyl rac-(2-(2-((tert-butyldimethylsilyl)oxy)-1-(dimethylamino)ethyl)pyridin-4-yl)carbamate (304 mg, 99%). ESI-MS m/z calc. 429.24478, found 428.7 (M−1)⁻; Retention time: 1.08 minutes.

### Step 4:

A suspension of benzyl rac-(2-(2-((tert-butyldimethylsilyl)oxy)-1-(dimethylamino)ethyl)pyridin-4-yl)carbamate (305 mg, 0.696 mmol) and palladium on carbon (100 mg of 10% w/w, 0.094 mmol) in EtOH (10 mL) was evacuated and refilled with nitrogen (×3), then hydrogen (×3) and stirred at ambient temperature for 1 h. The hydrogen atmosphere was replaced by N₂ and the mixture was filtered through a pre-wetted Celite cartridge, rinsing with MeOH (15 mL). The filtrates were concentrated in vacuo. Purification by flash chromatography (12 g SiO₂, 0 to 100% (3:1) EtOH/EtOAc containing 2% NH₄0H in heptane) gave rac-2-(2-((tert-butyldimethylsilyl)oxy)-1-(dimethylamino)ethyl)pyridin-4-amine (151 mg, 73%). ESI-MS m/z calc. 295.20798, found 294.5 (M−1)⁻; Retention time: 0.85 minutes.

### Intermediate CA

### 2-(ethylthio)pyridin-4-amine

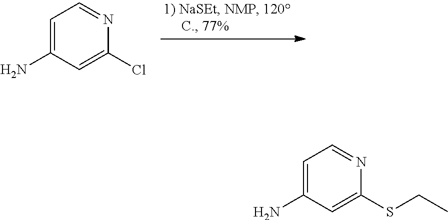

### Step 1:

A stirred mixture of 2-chloropyridin-4-amine (2 g, 15.56 mmol) and sodium ethanethiolate (3.15 g, 29.96 mmol) in NMP (20 mL) was heated at 120° C. for 65 h. The reaction was cooled to ambient temperature and partitioned between water and EtOAc. The layers were separated and the aqueous phase was extracted with EtOAc (×2). The combined organic extracts were washed with water (×2), brine (×2), dried (MgSO₄), filtered and concentrated in vacuo. Purification by flash chromatography (180 g SiO₂, 0 to 100% EtOAc in hexanes) gave 2-(ethylthio)pyridin-4-amine (1.847 g, 77%) as a pale yellow solid. ¹H NMR (400 MHz, DMSO-d₆) δ 7.84 (d, J=5.6 Hz, 1H), 6.33 (d, J=1.9 Hz, 1H), 6.24 (dd, J=5.7, 2.1 Hz, 1H), 5.98 (s, 2H), 3.00 (q, J=7.3 Hz, 2H), 1.25 (t, J=7.3 Hz, 3H) ppm. ESI-MS m/z calc. 154.05647, found 155.1 (M+1)⁺; Retention time: 0.54 minutes.

### Intermediate CB

### 1-bromo-4-(difluoromethyl)-3-fluoro-2-methoxybenzene

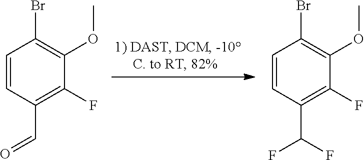

### Step 1:

DAST (24.4 g, 20 mL, 151.38 mmol) was added at −10° C. to a stirred solution of 4-bromo-2-fluoro-3-methoxybenzaldehyde (14 g, 60.077 mmol) in DCM (75 mL). The reaction mixture was stirred at ambient temperature for 16 h. The mixture was quenched with a mixture of ice cold water and solid NaHCO₃. The aqueous phase was separated and extracted with DCM (250 mL). The combined organic extracts were washed with water (50 mL), dried (Na₂SO₄), filtered and concentrated in vacuo. Purification by flash chromatography (SiO₂, 20 to 30% EtOAc in hexanes) gave 1-bromo-4-(difluoromethyl)-3-fluoro-2-methoxybenzene (12.6 g, 82%) as a green oil. ¹H NMR (400 MHz, DMSO-d₆) δ 7.62 (d, J=8.4 Hz, 1H), 7.34-7.07 (m, J=107.8 Hz, 2H), 3.91 (s, 3H) ppm.

### Intermediate CC

(3aR,6aS)-5-(2-chloroethyl)hexahydro-1H-furo[3,4-c]pyrrole

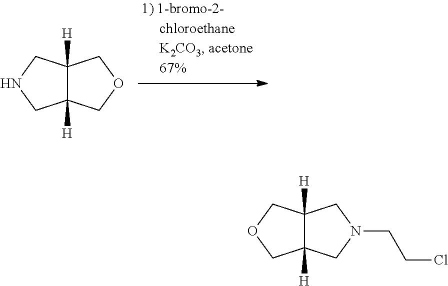 [SMILES: C1NC[C@H]2COC[C@@H]12 ; ClCCN1C[C@H]2COC[C@H]2C1]

### Step 1:

1-Bromo-2-chloroethane (1.35 mL, 16.29 mmol) was added to a stirred mixture of (3aR,6aS)-hexahydro-1H-furo[3,4-c]pyrrole (900 mg, 7.954 mmol) and K₂CO₃ (1.67 g, 12.08 mmol) in acetone (16 mL). The reaction mixture was stirred at ambient temperature for 48 h. The reaction mixture was filtered and concentrated in vacuo. Purification by flash chromatography (SiO₂, 0 to 100% (3:1) EtOAc/EtOH containing 0.5% NH₄OH in heptane) gave (3aR,6aS)-5-(2-chloroethyl)hexahydro-1H-furo[3,4-c]pyrrole (929 mg, 67%) as a yellow oil. ¹H NMR (500 MHz, Chloroform-d) δ 3.77-3.73 (m, 2H), 3.59-3.55 (m, 4H), 2.81 (d, J=5.1 Hz, 4H), 2.77 (t, J=6.9 Hz, 2H), 2.35 (d, J=5.1 Hz, 2H) ppm.

### Intermediate CD

(1r,3r)-3-((tert-butyldimethylsilyl)oxy)cyclobutyl methanesulfonate

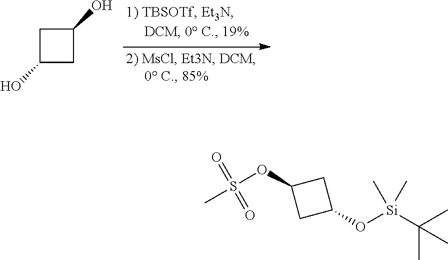 [SMILES: O[C@H]1C[C@H](O)C1 ; CC(C)(C)[Si](C)(C)O[C@H]1C[C@H](OS(C)(=O)=O)C1]

### Step 1:

tert-Butyldimethylsilyl trifluoromethanesulfonate (1.3 mL, 5.661 mmol) was added dropwise in 2 portions to a stirred suspension of (lr,3r)-cyclobutane-1,3-diol (500 mg, 5.675 mmol) and Et₃N (1.35 mL, 9.686 mmol) in DCM (15 mL) at 0° C. under nitrogen. The reaction mixture was stirred for 1 h. Further amounts of tert-butyldimethylsilyl trifluoromethanesulfonate (0.5 mL, 2.177 mmol; then 0.25 mL, 1.089 mmol) were added and the reaction mixture was stirred at 0° C. under nitrogen for 1 h then for 30 min. The mixture was quenched by addition of water (15 mL), diluted with DCM (5 mL) and stirred vigorously under nitrogen at 0° C. for 30 min. The organic phases were passed through a phase separator cartridge and the filtrate was concentrated in vacuo. Purification by flash chromatography (24 g SiO₂, 0 to 40% EtOAc in hexanes) gave (1r,3r)-3-((tert-butyldimethylsilyl)oxy)cyclobutan-1-ol (218 mg, 19%) as a colourless oil. ¹H NMR (500 MHz, Chloroform-d) δ 4.58 (p, J=6.2 Hz, 1H), 4.53-4.46 (m, 1H), 2.22 (dd, J=6.2, 5.0 Hz, 4H), 0.88 (s, 9H), 0.03 (s, 6H) ppm; OH alcohol not observed.

### Step 2:

MsCl (160 μL, 2.067 mmol) was added to a stirred solution of (1r,3r)-3-((tert-butyldimethylsilyl)oxy)cyclobutan-1-ol (218 mg, 1.077 mmol) and Et₃N (450 μL, 3.229 mmol) in DCM (9 mL) at 0° C. The reaction mixture was slowly warmed to ambient temperature under nitrogen over 16 h. The mixture was diluted with water (15 mL) and DCM (5 mL) and stirred vigorously for 30 min. The organic phase was passed through a phase separator cartridge and the filtrate was concentrated in vacuo to give (1r,3r)-3-((tert-butyldimethylsilyl)oxy)cyclobutyl methanesulfonate (302 mg, 85%) as a pale yellow oil. ¹H NMR (500 MHz, Chloroform-d) δ 5.18 (tt, J=7.2, 3.8 Hz, 1H), 4.57 (tt, J=7.0, 5.0 Hz, 1H), 2.98 (s, 3H), 2.63-2.53 (m, 2H), 2.39 (dddd, J=13.9, 6.9, 3.4, 1.2 Hz, 2H), 0.88 (s, 9H), 0.04 (s, 6H) ppm.

### Intermediate CE

(2-iodoethoxy)triisopropylsilane

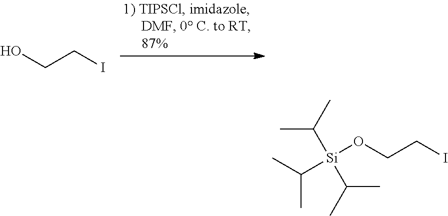

### Step 1:

TIPSCl (16.0 mL, 75.52 mmol) was added dropwise over 10 min to a stirred solution of 2-iodoethanol (10 g, 58.15 mmol) and imidazole (5.2 g, 76.38 mmol) in DMF (30 mL) cooled in an ice bath. The mixture was stirred in the ice bath for 1 h then at ambient temperature for a further 1 h 45 at which time a suspension had formed. The mixture was partitioned between water (40 mL) and MTBE (20 mL). The organic layer was separated and filtered through a silica gel pad washing through with heptane. The filtrate was concentrated in vacuo to give (2-iodoethoxy)triisopropylsilane (22.05 g, 87%) as a pale yellow oil. ¹H NMR (500 MHz, DMSO-d₆) δ 3.88 (t, J=6.1 Hz, 2H), 3.32 (t, J=6.1 Hz, 2H), 1.16-0.87 (m, 21H) ppm.

The following intermediate was made using a method similar to that described in Intermediate CE except that tert-butylchlorodimethylsilane was used in place of TIPSCl:

| | | | |
|---|---|---|---|
| Intermediate | | | |
| No. | Compound Name | LC/MS | NMR (shifts in ppm) |

| | | |
|---|---|---|
| CF | ((1r,3r)-3- | ¹H NMR (500 MHz, |
| | bromocyclobutoxy)(tert- | Chloroform-d) δ |
| | butyl)dimethylsilane | 4.79-4.71 (m, 1H), |
| | | 4.44 (tt, J = 7.3, 3.5 |
| | | Hz, 1H), 2.70-2.55 (m, |
| | | 4H), 0.88 (s, 9H), 0.04 |
| | | (s, 6H) ppm. |

The following intermediate was made using a method similar to that described in Intermediate 29 except that tert-butylchlorodimethylsilane was used in place of TIPSCl and the reaction was carried out at ambient temperature:

| | | | |
|---|---|---|---|
| Intermediate | | | |
| No. | Compound Name | LC/MS | NMR (shifts in ppm) |

| | | |
|---|---|---|
| CG | 2-(((tert- | ¹H NMR (400 MHz, |
| | butyldimethylsilyl)oxy)methyl)pyridin- | DMSO-d₆) δ 7.86 (d, J = |
| | 4-amine | 5.5 Hz, 1H), 6.60-6.53 |
| | | (m, 1H), 6.30 (dd, J = 5.6, |
| | | 2.3 Hz, 1H), 5.99 (s, 2H), |
| | | 4.52 (s, 2H), 0.92 (s, 9H), |
| | | 0.08 (s, 6H) ppm. |

### Intermediate CH

(R)-2-(2-oxa-6-azaspiro[3.3]heptan-6-yl)propan-1-ol

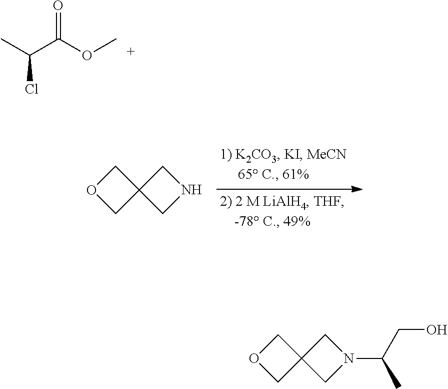

### Step 1:

Potassium carbonate (11 g, 79.591 mmol) was added to a suspension of 2-oxa-6-azaspiro[3.3]heptane (4 g, 40.351 mmol), methyl (S)-2-chloropropanoate (4.95 g, 40.392 mmol) and potassium iodide (0.5 g, 3.0120 mmol) in acetonitrile (50 mL). The mixture was stirred at 65° C. for 15 h. The reaction mixture was partitioned between ethyl acetate (100 mL) and water (25 mL). The organic layer was separated and washed with brine (20 mL), dried (Na₂SO₄), filtered and concentrated in vacuo to give methyl (R)-2-(2-oxa-6-azaspiro[3.3]heptan-6-yl)propanoate (5.1 g, 61%) as a yellow oil. ¹H NMR (300 MHz, Chloroform-d) δ 4.79-4.71 (m, 4H), 3.70 (s, 3H), 3.44 (d, J=7.2 Hz, 4H), 2.99 (q, J=6.9 Hz, 1H), 1.18 (d, J=6.9 Hz, 3H) ppm.

### Step 2:

2 M LiAlH₄ (7.5 mL, 2 M in THF, 15.0 mmol) was added slowly to a solution of methyl (R)-2-(2-oxa-6-azaspiro[3.3]heptan-6-yl)propanoate (1.5 g, 7.289 mmol) in THF (10 mL) at −78° C. The reaction was stirred at −78° C. for 1 h. The mixture was quenched at −78° C. by addition of sodium sulfate decahydrate. The mixture was warmed up to ambient temperature over 6 h and filtered through a pad of Celite, rinsing with ethyl acetate (30 mL). The filtrate was concentrated in vacuo to give (R)-2-(2-oxa-6-azaspiro[3.3]heptan-6-yl)propan-1-ol (620 mg, 49%) as a colourless oil, which solidified on standing. ¹H NMR (400 MHz, Chloroform-d) δ 4.74 (d, J=11.4 Hz, 4H), 3.49 (dd, J=10.8, 3.9 Hz, 1H), 3.40-3.31 (m, 5H), 2.32-2.25 (m, 1H), 2.08-1.90 (br s, 1H), 0.94 (d, J=6.4 Hz, 3H) ppm.

The following intermediate was made using a method similar to that described in Intermediate CH except that methyl (R)-2-chloropropanoate was used in place of methyl (S)-2-chloropropanoate in Step 1:

| | | | |
|---|---|---|---|
| Intermediate | | | |
| No. | Compound Name | LC/MS | NMR (shifts in ppm) |

| | | |
|---|---|---|
| CI | (S)-2-(2-oxa-6- | ¹H NMR (400 MHz, |
| | azaspiro[3.3]heptan- | Chloroform-d) δ 4.74 (d, |
| | 6-yl)propan-1-ol | J = 11.7 Hz, 4H), 3.50 |
| | | (dd, J = 11.0, 4.1 Hz, |
| | | 1H), 3.40-3.31 (m, 5H), |
| | | 2.32-2.25 (m, 1H), |
| | | 2.05-2.01 (br s, 1H), 0.94 |
| | | (d, J = 6.4 Hz, 3H) ppm. |

### Intermediate CJ

(S)-1-methoxypropan-2-yl 4-methylbenzenesulfonate

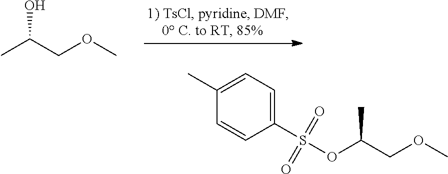

### Step 1:

TsCl was added portionwise to a stirred solution of (S)-1-methoxypropan-2-ol in pyridine (1 mL) cooled in an ice bath. The reaction mixture was warmed gradually to ambient temperature and stirred overnight. The mixture was partitioned between 1 M HCl (10 mL) and DCM (10 mL). The organic phase was separated and washed with a saturated sodium bicarbonate solution (25 mL), passed through a phase separator cartridge and the filtrate was concentrated in vacuo to give (5)-1-methoxypropan-2-yl 4-methylbenzenesulfonate (231 mg, 85%). ¹H NMR (500 MHz, DMSO-d₆) δ 7.84-7.75 (m, 2H), 7.53-7.44 (m, 2H), 4.67 (qdd, J=6.4, 5.8, 3.7 Hz, 1H), 3.41-3.29 (m, 2H), 3.14 (s, 3H), 2.43 (d, J=0.7 Hz, 3H), 1.15 (d, J=6.5 Hz, 3H) ppm.

### Intermediate CK

### 3-bromo-6-(difluoromethyl)-2-methoxypyridine

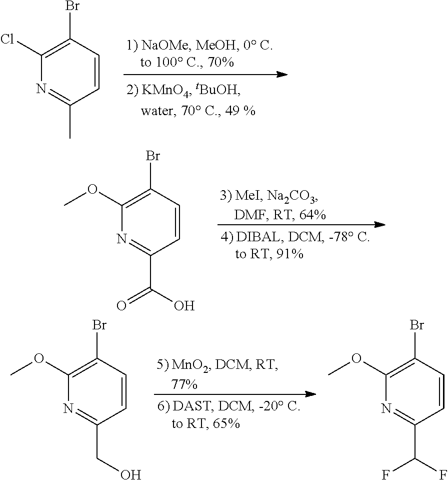

### Step 1:

Sodium methoxide (20 mL, 25% w/v solution in MeOH, 92.552 mmol) was added at 0° C. to a stirred solution of 3-bromo-2-chloro-6-methylpyridine (8 g, 38.747 mmol) in MeOH (50 mL) in a sealed tube. The reaction mixture was heated at 100° C. for 16 h. The reaction mixture was concentrated in vacuo. The residue was diluted with water (100 mL) and extracted with EtOAc (3×100 mL). The combined organic layers were dried over Na₂SO₄, filtered, and concentrated in vacuo to give 3-bromo-2-methoxy-6-methylpyridine (5.5 g, 70%) as a colourless oil. ¹H NMR (400 MHz, DMSO-d₆) δ 7.82 (d, J=7.7 Hz, 1H), 6.76 (d, J=7.7 Hz, 1H), 3.88 (s, 3H), 2.35 (s, 3H) ppm. ESI-MS m/z calc. 200.9789, found 202.01 (M+1)⁺; Retention time: 1.69 minutes.

### Step 2:

KMnO₄ (13 g, 82.261 mmol) was added at ambient temperature to a stirred solution of 3-bromo-2-methoxy-6-methylpyridine (5.5 g, 27.221 mmol) in tert-butanol (150 mL) and water (300 mL). The reaction mixture was heated at 70° C. for 16 h. The reaction mixture was quenched by addition of a 1M aqueous solution of HCl (80 mL). The resulting mixture was stirred for 30 min, filtered and extracted with EtOAc (2×100 mL). The mother liquors were extracted with EtOAc (2×50 mL). The combined organic layers were washed with a 0.5 N aqueous solution of NaOH (2×100 mL). The aqueous layer was collected, acidified by addition of a 12N aqueous solution of HCl and extracted with DCM (2×100 mL). The combined organic extracts were washed with brine, dried over Na₂SO₄, filtered, and concentrated in vacuo to give 5-bromo-6-methoxypyridine-2-carboxylic acid (3.1 g, 49%) as white solid. ¹H NMR (400 MHz, DMSO-d₆) δ 13.27 (br s, 1H), 8.19 (d, J=7.9 Hz, 1H), 7.57 (d, J=7.9 Hz, 1H), 3.98 (s, 3H) ppm. ESI-MS m/z calc. 230.9531, found 232.0 (M+1)⁺; Retention time: 1.34 minutes.

### Step 3:

Sodium carbonate (1.5 g, 14.153 mmol) was added to a stirred solution of 5-bromo-6-methoxypyridine-2-carboxylic acid (3 g, 12.929 mmol) in DMF (40 mL). Methyl iodide (3.8760 g, 1.7 mL, 27.308 mmol) was added and the mixture was stirred at ambient temperature for 16 h. The reaction mixture was quenched by addition of ice-cold water (50 mL). The aqueous phase was extracted with ethyl acetate (3×50 mL). The combined organic layers were washed with water (2×100 mL), brine (50 mL), dried over Na₂SO₄, filtered, and concentrated in vacuo to give methyl 5-bromo-6-methoxypyridine-2-carboxylate (2.02 g, 64%) as an off-white solid. ¹H NMR (400 MHz, DMSO-d₆) δ 8.22 (d, J=7.8 Hz, 1H), 7.59 (d, J=7.8 Hz, 1H), 3.98 (s, 3H), 3.87 (s, 3H) ppm. ESI-MS m/z calc. 244.9688, found 246.1 (M+1)⁺; Retention time: 3.21 minutes.

### Step 4:

Diisobutylaluminum hydride (14 mL, 25% w/v solution in toluene, 24.610 mmol) was added at −78° C. to a stirred solution of methyl 5-bromo-6-methoxypyridine-2-carboxylate (2 g, 8.128 mmol) in DCM (80 mL). The reaction mixture was stirred at ambient temperature for 1 h. The reaction mixture was quenched by addition of a saturated aqueous solution of sodium tartrate (50 mL). The mixture was stirred for 30 min then extracted with DCM (3×100 mL). The combined organic layers were dried over Na₂SO₄, filtered, and concentrated in vacuo to give (5-bromo-6-methoxy-2-pyridyl)methanol (1.62 g, 91%) as a white solid. ¹H NMR (400 MHz, DMSO-d₆) δ 7.99 (d, J=7.8 Hz, 1H), 7.00 (d, J=7.6 Hz, 1H), 5.45 (t, J=11.8 Hz, 1H), 4.45 (d, J=5.9 Hz, 2H), 3.89 (s, 3H) ppm. ESI-MS m/z calc. 216.9738, found 218.0 (M+1)⁺; Retention time: 2.93 minutes.

### Step 5:

MnO₂ (8 g, 92.021 mmol) was added to a stirred solution of (5-bromo-6-methoxy-2-pyridyl)methanol (1.6 g, 7.3378 mmol) in DCM (80 mL). The reaction mixture was stirred at ambient temperature for 16 h. The reaction mixture was filtered and concentrated in vacuo to give 5-bromo-6-methoxypyridine-2-carbaldehyde (1.22 g, 77%) as off-white solid. ¹H NMR (400 MHz, DMSO-d₆) δ 9.88 (s, 1H), 8.29 (d, J=7.7 Hz, 1H), 7.49 (d, J=7.7 Hz, 1H), 4.03 (s, 3H) ppm.

### Step 6:

DAST (1.9740 g, 1.5 mL, 12.246 mmol) was slowly added at −20° C. to a stirred solution of 5-bromo-6-methoxypyridine-2-carbaldehyde (1.2 g, 5.5547 mmol) in DCM (30.000 mL). The reaction mixture was stirred at ambient temperature for 16 h. The reaction mixture was quenched by addition of ice-water. The pH of the solution was adjusted to 8-10 by addition of solid sodium hydrogen carbonate. The organic phase was collected, washed with water and brine, dried over Na₂SO₄, filtered, and concentrated in vacuo. Purification by flash chromatography (SiO₂, 100% hexanes) gave 3-bromo-6-(difluoromethyl)-2-methoxypyridine (900 mg, 65%) as a pale yellow oil. ¹H NMR (400 MHz, DMSO-d₆) δ 8.22 (d, J=7.8 Hz, 1H), 7.23 (d, J=7.7 Hz, 1H), 7.03-6.75 (m, 1H), 3.96 (s, 3H) ppm.

### Intermediate CL

### 3-methyl-1-(methylsulfonyl)-1H-pyrazol-4-amine

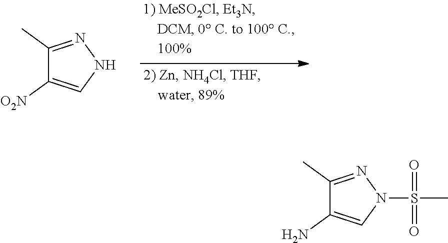

### Step 1:

Methanesulfonyl chloride (66.6 g, 45 mL, 581.4 mmol) was added dropwise over 1 h to a stirred solution of 3-methyl-4-nitro-1H-pyrazole (50 g, 385.52 mmol) and Et₃N (79.86 g, 110 mL, 789.21 mmol) in DCM (1.5 L) at 0° C. The reaction mixture was stirred at ambient temperature overnight. The mixture was quenched by addition of a saturated ammonium chloride solution (1 L). The aqueous was separated and extracted with DCM (3×500 mL). The combined organic extracts were washed with brine, dried (Na₂SO₄), filtered and concentrated in vacuo to give 3-methyl-1-(methylsulfonyl)-4-nitro-1H-pyrazole (82 g, 100%) as brown solid. ¹H NMR (500 MHz, Chloroform-d) δ 8.74 (s, 1H), 3.45 (s, 3H), 2.64 (d, J=0.5 Hz, 3H) ppm.

### Step 2:

Zn (64 g, 8.972 mL, 978.74 mmol) was added portionwise over 30 min to a mixture of 3-methyl-1-(methylsulfonyl)-4-nitro-1H-pyrazole (55 g, 254.64 mmol) and ammonium chloride (52 g, 33.987 mL, 972.12 mmol) in a mixture of THF (1 L) and water (500 mL). The reaction mixture was stirred overnight at ambient temperature. The mixture was filtered through a pad of Celite, washing with ethyl acetate (3×500 mL). The aqueous layer was separated and extracted with ethyl acetate (2×500 mL). The combined organic extracts were washed with brine, dried (Na₂SO₄), filtered and concentrated in vacuo. The resulting yellow solid was triturated with a mixture of ethyl acetate and hexanes (1:1). The solid was filtered and dried to give 3-methyl-1-(methylsulfonyl)-1H-pyrazol-4-amine (41.6 g, 89%) as a yellow solid. ¹H NMR (500 MHz, DMSO-d₆) δ 7.23 (s, 1H), 4.39 (s, 2H), 3.23 (s, 3H), 2.13 (s, 3H) ppm.

### Intermediate CM

### rac-2-methyl-5-(tetrahydrofuran-3-yl)pyridin-3-amine

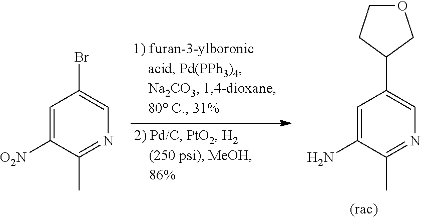

### Step 1:

5-Bromo-2-methyl-3-nitropyridine (5 g, 23.0 mmol) and furan-3-ylboronic acid (2.85 g, 25.3 mmol) were dissolved in 1,4-dioxane (75 mL) and purged with nitrogen for several minutes. During the purge, a 2 M sodium carbonate solution (20 mL) was added, followed by Pd(PPh₃)₄(2.7 g, 2.34 mmol). The flask was sealed and the reaction was heated in a sand bath to 80° C. for 3 h. The reaction mixture was cooled and the solvent was removed in vacuo. The residue was partitioned between water and dichloromethane and the organic phase was washed with brine, dried (Na₂SO₄), filtered and concentrated in vacuo. Purification by flash chromatography (SiO₂, 0 to 100% dichloromethane containing 10% methanol in dichloromethane; then SiO₂, 0 to 100% dichloromethane containing 5% methanol in dichloromethane) gave 5-(furan-3-yl)-2-methyl-3-nitropyridine (1.8 g, 31%) as a white solid. ¹H NMR (400 MHz, DMSO-d₆) δ 9.08 (d, J=2.0 Hz, 1H), 8.62 (d, J=2.1 Hz, 1H), 8.48 (s, 1H), 7.84 (t, J=1.7 Hz, 1H), 7.25-7.10 (m, 1H), 2.73 (s, 3H) ppm.

### Step 2:

5-(Furan-3-yl)-2-methyl-3-nitropyridine (1.8 g, 8.82 mmol) was dissolved in methanol (100 mL) treated with 10% Pd/C (500 mg) and PtO₂ (500 mg). The mixture was placed in a steel bomb and subjected to 250 psi hydrogen for 12 h. The pressure at the end of the reaction was reduced by -75-80 psi during the course of the reaction. The crude mixture of product and starting material was isolated. New portions of catalyst were added, and the hydrogenation was continued at 250 psi of hydrogen. A 50 psi reduction in pressure was noted from the initial pressure. The reaction mixture was filtered through a pad of Celite, rinsing with methanol. The filtrates were concentrated in vacuo. The residue was azeotroped with chloroform to give rac-2-methyl-5-(tetrahydrofuran-3-yl)pyridin-3-amine (1.7 g, 86%), which contained -10% chloroform. ¹H NMR (400 MHz, Chloroform-d) δ 7.84 (d, J=1.8 Hz, 1H), 6.85 (d, J=1.8 Hz, 1H), 4.90 (s, 2H), 4.17-3.98 (m, 2H), 3.98-3.81 (m, 1H), 3.71 (dd, J=8.5, 6.8 Hz, 1H), 3.40-3.25 (m, 1H), 2.48-2.28 (m, 1H), 2.41 (s, 3H), 1.95 (dq, J=12.4, 7.8 Hz, 1H) ppm.

### Intermediate CN

### 1-((1s,3s)-3-fluorocyclobutyl)-1H-1,2,4-triazol-3-amine

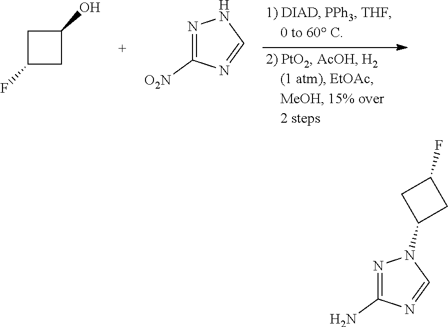 [SMILES: O[C@H]1C[C@H](F)C1 ; O=[N+]([O-])c1nc[nH]n1 ; Nc1ncn([C@H]2C[C@@H](F)C2)n1]

### Step 1:

DIAD (1.773 g, 1.726 mL, 8.767 mmol) was added over 20 min to a stirred mixture of triphenylphosphane (2.299 g, 8.767 mmol), 3-nitro-1H-1,2,4-triazole (1 g, 8.767 mmol) and 3-fluorocyclobutanol (877.5 mg, 9.740 mmol) in THF (6.5 mL) cooled in an ice bath. The ice bath was removed and the reaction mixture was heated at 60° C. for 5 h 30 min. The mixture was concentrated in vacuo. Purification by flash chromatography (80 g SiO₂, 10 to 50% EtOAc in heptane) gave 1-((1s,3s)-3-fluorocyclobutyl)-3-nitro-1H-1,2,4-triazole (816 mg) which contained some 3-nitro-1H-1,2,4-triazole and which was used without further purification in the next step. ¹H NMR (400 MHz, DMSO-d₆) δ 8.97 (s, 1H), 5.05 (dp, J=56.3, 6.6 Hz, 1H), 4.75 (qd, J=8.9, 1.8 Hz, 1H), 3.03 (tdd, J=14.0, 8.6, 5.1 Hz, 2H), 2.81-2.66 (m, 2H) ppm. ESI-MS m/z calc. 186.0553, found 187.12 (M+1)⁺; Retention time: 0.61 minutes.

### Step 2:

A mixture of 1-((1s,3s)-3-fluorocyclobutyl)-3-nitro-1H-1,2,4-triazole (816 mg, 4.384 mmol) and PtO₂ (80 mg, 0.3523 mmol) in MeOH (5 mL), AcOH (1 mL) and EtOAc (1 mL) was stirred overnight under a hydrogen atmosphere. The reaction mixture was concentrated in vacuo. Purification by column chromatography (40 g SiO₂, 0 to 20% MeOH in DCM) gave 1-((1s,3s)-3-fluorocyclobutyl)-1H-1,2,4-triazol-3-amine (105 mg, 15% over 2 steps) as a white solid. ¹H NMR (400 MHz, DMSO-d₆) δ 8.01 (s, 1H), 5.33 (s, 2H), 5.07-4.82 (m, 1H), 4.38-4.25 (m, 1H), 2.90-2.74 (m, 2H), 2.72-2.55 (m, 2H) ppm. ESI-MS m/z calc. 156.08113, found 157.06 (M+1)⁺; Retention time: 0.28 minutes.

### Intermediate CO

### 3-methyl-[1,2,3]triazolo[1,5-a]pyridin-5-amine

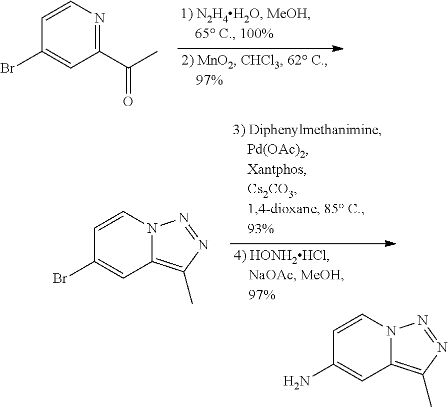

### Step 1:

Hydrazine monohydrate (4.59 g, 4.5 mL, 59.598 mmol) was added to a stirred solution of 1-(4-bromopyridin-2-yl)ethan-1-one (10 g, 49.992 mmol) in methanol (140 mL) and the reaction mixture was heated at 65° C. for 3 h. The mixture was cooled to ambient temperature, diluted with dichloromethane (50 mL), dried (Na₂SO₄), filtered and concentrated in vacuo to give 4-bromo-2-(1-hydrazineylideneethyl)pyridine (10.66 g, 100%) as beige solid, which was used without further purification in the next step. ¹H NMR (300 MHz, Chloroform-d) δ 8.34 (d, J=5.3 Hz, 1H), 8.14 (d, J=2.1 Hz, 1H), 7.33 (dd, J=5.0, 1.8 Hz, 1H), 5.62 (br s, 2H), 2.23 (s, 3H) ppm. ESI-MS m/z calc. 212.9902, found 214.1 (M+1)⁺; Retention time: 1.08 minutes.

### Step 2:

Manganese dioxide (activated) (12 g, 117.33 mmol) was added to a stirred solution of 4-bromo-2-(1-hydrazineylideneethyl)pyridine (10.66 g, 49.798 mmol) in chloroform (100 mL) and reaction was heated at 62° C. overnight. The mixture was cooled to ambient temperature, filtered through a pad of Celite, rinsing with dichloromethane. The mother liquors were concentrated in vacuo to give 5-bromo-3-methyl-[1,2,3]triazolo[1,5-a]pyridine (10.28 g, 97%) as a beige solid. ¹H NMR (300 MHz, Chloroform-d) δ 8.51 (d, J=7.6 Hz, 1H), 7.84-7.76 (m, 1H), 6.99 (dd, J=7.3, 1.8 Hz, 1H), 2.59 (s, 3H) ppm. ESI-MS m/z calc. 210.9745, found 212.0 (M+1)⁺; Retention time: 2.07 minutes.

### Step 3:

Nitrogen was bubbled through 1,4-dioxane (75 mL) nitrogen for 15 min. Cesium carbonate (12.3 g, 37.751 mmol), 5-bromo-3-methyl-[1,2,3]triazolo[1,5-a]pyridine (4 g, 18.845 mmol), xantphos (1.30 g, 2.2467 mmol), diphenylmethanimine (4.4280 g, 4.1 mL, 24.433 mmol) and palladium acetate (230 mg, 1.0245 mmol) were successively added to the flask. The reaction mixture was heated at 85° C. overnight. The mixture was cooled to ambient temperature, filtered through a pad of Celite, rinsing with ethyl acetate. The mother liquors were concentrated in vacuo. Purification by column chromatography (SiO₂, 0 to 40% EtOAc in heptane) gave N-(3-methyl-[1,2,3]triazolo[1,5-a]pyridin-5-yl)-1,1-diphenylmethanimine (5.49 g, 93%) as yellow solid. ¹H NMR (300 MHz, Chloroform-d) δ 8.38 (d, J=7.3 Hz, 1H), 7.77 (d, J=7.3 Hz, 2H), 7.56-7.40 (m, 3H), 7.36-7.28 (m, 3H), 7.16 (d, J=6.2 Hz, 2H), 6.82 (d, J=1.2 Hz, 1H), 6.40 (dd, J=7.5, 1.9 Hz, 1H), 2.47 (s, 3H) ppm. ESI-MS m/z calc. 312.1375, found 313.2 (M+1)⁺; Retention time: 2.04 minutes.

### Step 4:

Sodium acetate (3.84 g, 46.810 mmol) and hydroxylamine hydrochloride (2.52 g, 36.264 mmol were successively added to a solution ofN-(3-methyl-[1,2,3]triazolo[1,5-a]pyridin-5-yl)-1,1-diphenylmethanimine (5.49 g, 17.575 mmol) in methanol (175 mL). The reaction mixture was stirred at ambient temperature for 1 h. The pH of the solution was adjusted to 7-8 by addition of aqueous ammonium hydroxide. Silica gel was added and reaction mixture was concentrated in vacuo. Purification by column chromatography (SiO₂, 50 to 100% EtOAc in heptane) gave 3-methyl-[1,2,3]triazolo[1,5-a]pyridin-5-amine (2.52 g, 97%) as a pink solid. ¹H NMR (300 MHz, DMSO-d₆) δ 8.56 (d, J=7.0 Hz, 1H), 6.52 (dd, J=7.6, 2.3 Hz, 1H), 6.42 (dd, J=2.3, 0.9 Hz, 1H), 5.89 (s, 2H), 2.31 (s, 3H) ppm. ESI-MS m/z calc. 148.0749, found 149.2 (M+1)⁺; Retention time: 0.92 minutes.

The following intermediate was made using a method similar to that described in Intermediate CO except that Steps 1 and 2 were omitted. 6-Bromo-1-methyl-1H-[1,2,3]triazolo[4,5-b]pyridine, starting material of Step 3, was prepared at ambient temperature in 11% yield by methylation of 6-bromo-1H-[1,2,3]triazolo[4,5-b]pyridine in the presence of methyl iodide, using sodium bis(trimethylsilyl)amide (1 M solution in THF) as the base and DMSO as the solvent:

| Intermediate | | | |
|---|---|---|---|
| No. | Compound Name | LC/MS | NMR (shifts in ppm) |
| CP | 1-methyl-1H- | ESI-MS m/z calc. | ¹H NMR (400 MHz, |
| | [1,2,3]triazolo[4,5-b]pyridin-6- | 149.0701, found 150.2 | DMSO-d₆) δ 8.12 (d, J = |
| | amine | (M + 1)⁺; Retention time: | 2.3 Hz, 1H), 6.94 (d, J = |
| | | 0.52 minutes | 2.6 Hz, 1H), 5.95 (s, 2H), |
| | | | 4.10 (s, 3H) ppm. |

The following intermediate was made using a method similar to that described in Intermediate CO except that Steps 1 and 2 were omitted. 6-bromo-[1,2,3]triazolo[1,5-a]pyridine was used as the starting material of Step 3 in place of 5-bromo-3-methyl-[2,3]triazolo[1,5-a]pyridine;

| Intermediate | | | |
|---|---|---|---|
| No. | Compound Name | LC/MS | NMR (shifts in ppm) |
| CQ | [1,2,3]triazolo[1,5-a]pyridin- | ESI-MS m/z calc. | ¹H NMR (400 MHz, |
| | 6-amine | 134.0592, found 134.99 | DMSO-d₆) δ 8.07 (s, 1H), |
| | | (M + 1)⁺; Retention time: | 7.91 (d, J = 0.9 Hz, 1H), |
| | | 0.3 minutes | 7.65 (d, J = 9.6 Hz, 1H), |
| | | | 6.95 (dd, J = 9.2, 1.8 Hz, |
| | | | 1H), 5.42 (s, 2H) ppm. |

### Intermediate CR

### 4-amino-3-methyl-1H-pyrazole-1-carboxamide

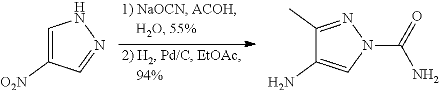

### Step 1:

A solution of sodium cyanate (7.68 g, 118.14 mmol) in water (25 mL) was added to a mixture of 3-methyl-4-nitro-1H-pyrazole (10 g, 78.678 mmol) in acetic acid (150 mL) and water (25 mL). The reaction mixture was stirred at 25° C. for 18 h. A further amount of sodium cyanate (5.11 g, 78.607 mmol) was added and the solution was stirred at ambient temperature for 18 h. The mixture was diluted with water and the solid formed was filtered. The solid was partitioned between ethyl acetate (100 mL) and water (100 mL). The aqueous phase was separated and extracted with ethyl acetate (2×100 mL). The combined organic extracts were washed with brine (100 mL), dried (Na₂SO₄), filtered and concentrated in vacuo to give 3-methyl-4-nitro-1H-pyrazole-1-carboxamide (7.43 g, 55%) as a white solid. ¹H NMR (300 MHz, Chloroform-d) δ 8.88 (s, 1H), 7.08 (br s, 1H), 5.58 (br s, 1H), 2.58 (s, 3H) ppm.

### Step 2:

Palladium on carbon (1.15 g, 0.5403 mmol) was added to a solution of 3-methyl-4-nitro-1H-pyrazole-1-carboxamide (7.43 g, 43.237 mmol) in ethyl acetate (200 mL) under nitrogen. Hydrogen was bubbled through the solution for 5 min and the reaction mixture was stirred under a hydrogen atmosphere (balloon) overnight at ambient temperature. The mixture was filtered on Celite, washed with ethyl acetate (2×50 mL). Palladium on carbon (1.15 g, 0.5403 mmol) was added to the solution under a nitrogen atmosphere. Hydrogen was bubbled through the solution for 5 min. The mixture was stirred under a hydrogen atmosphere for 18 h. The mixture was filtered on a pad of Celite, washing with ethyl acetate (2×50 mL). The mother liquors were concentrated in vacuo to give 4-amino-3-methyl-1H-pyrazole-1-carboxamide (5.77 g, 94%) as a pink solid. ¹H NMR (300 MHz, Chloroform-d) δ 7.61 (s, 1H), 6.86 (br s, 1H), 5.06 (br s, 1H), 2.98 (br s, 2H), 2.20 (s, 3H) ppm. ESI-MS m/z calc. 140.0698, found 141.2 (M+1)⁺; Retention time: 0.27 minutes.

### Intermediate CS

### rac-2-(1-((tert-butyldimethylsilyl)oxy)-2-methoxyethyl)pyridin-4-amine

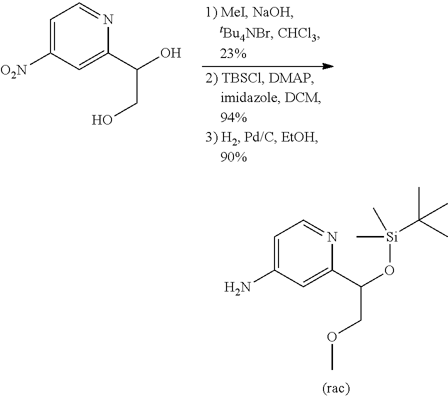 [SMILES: O=[N+]([O-])c1ccnc(C(O)CO)c1 ; COCC(O[Si](C)(C)C(C)(C)C)c1cc(N)ccn1]

### Step 1:

NaOH (22 mL, 1 M solution in water, 22.0 mmol), methyl iodide (2.964 g, 1.3 mL, 20.882 mmol) and ^{t}Bu₄NBr (350 mg, 1.0857 mmol) were successively added to a stirred solution of 1-(4-nitropyridin-2-yl)ethane-1,2-diol (2 g, 10.861 mmol) in chloroform (5 mL). The reaction mixture was stirred overnight at ambient temperature. The aqueous phase was separated and extracted with DCM. The combined organic extracts were dried (Na₂SO₄), filtered and concentrated in vacuo. Purification by column chromatography (SiO₂, 50% EtOAc in hexanes) gave rac-2-methoxy-1-(4-nitropyridin-2-yl)ethan-1-ol (490 mg, 23%) as a yellow solid. ¹H NMR (400 MHz, Chloroform-d) δ 8.83 (d, J=5.32 Hz, 1H), 8.23 (d, J=1.76 Hz, 1H), 7.93-7.92 (m, 1H), 5.03 (s, 1H), 4.13-4.07 (m, 1H), 3.78-3.74 (m, 1H), 3.68 (s, 1H), 3.39 (s, 3H) ppm.

### Step 2:

TBSCl (600 mg, 3.981 mmol) was added to a stirred solution of rac-2-methoxy-1-(4-nitropyridin-2-yl)ethan-1-ol (400 mg, 2.018 mmol), imidazole (400 mg, 5.876 mmol) and DMAP (25 mg, 0.205 mmol) in DCM (13 mL) and the mixture was stirred at ambient temperature overnight. The mixture was partitioned between water and DCM. The aqueous layer was separated and extracted with DCM. The combined organic extracts were washed with brine and water, dried (Na₂SO₄), filtered and concentrated in vacuo. Purification by column chromatography (SiO₂, 10% EtOAc in hexanes) gave rac-2-(1-((tert-butyldimethylsilyl)oxy)-2-methoxyethyl)-4-nitropyridine (595 mg, 94%) as a colourless oil. ¹H NMR (400 MHz, Chloroform-d) δ 8.81 (d, J=5.4 Hz, 1H), 8.29 (d, J=2.3 Hz, 1H), 7.88 (dd, J=5.4, 2.2 Hz, 1H), 5.06 (dd, J=6.0, 3.7 Hz, 1H), 3.69 (dd, J=10.2, 3.8 Hz, 1H), 3.61 (dd, J=10.1, 6.0 Hz, 1H), 3.35 (s, 3H), 0.93 (s, 9H), 0.13 (s, 3H), 0.01 (s, 3H) ppm.

### Step 3:

A solution of rac-2-(1-((tert-Butyldimethylsilyl)oxy)-2-methoxyethyl)-4-nitropyridine (3.2 g, 10.242 mmol) in ethanol (100 mL) was degassed for 5 min. Pd/C (1.6 g, 10% w/w, 1.317 mmol) was added and the reaction mixture was stirred for 4 h under a hydrogen atmosphere (balloon). The mixture was filtered through a pad of Celite. The mother liquors were concentrated in vacuo. Purification by column chromatography (SiO₂, 50 to 70% EtOAc in hexanes) gave rac-2-(1-((tert-butyldimethylsilyl)oxy)-2-methoxyethyl)pyridin-4-amine (2.6 g, 90%) as a white solid. ¹H NMR (400 MHz, Chloroform-d) δ 8.12 (d, J=5.5 Hz, 1H), 6.78 (d, J=2.4 Hz, 1H), 6.38 (dd, J=5.5, 2.4 Hz, 1H), 4.84 (dd, J=6.9, 3.2 Hz, 1H), 4.11 (s, 2H), 3.63 (dd, J=10.3, 3.2 Hz, 1H), 3.50 (dd, J=10.3, 6.8 Hz, 1H), 3.34 (s, 3H), 0.90 (s, 9H), 0.09 (s, 3H), 0.03 (s, 3H) ppm. ESI-MS m/z calc. 282.1764, found 283.0 (M+1)⁺; Retention time: 1.42 minutes.

### Intermediate CT

(R)-1-(5-aminopyridin-3-yl)-4,6-dimethylpiperazin-2-one

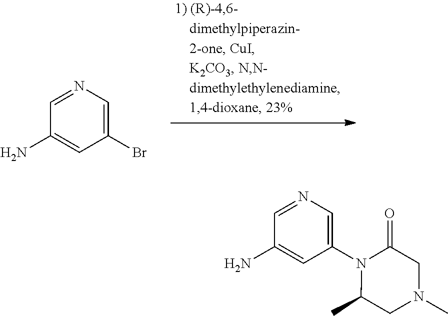 [SMILES: Nc1cncc(Br)c1 ; C[C@@H]1CN(C)CC(=O)N1c1cncc(N)c1]

### Step 1:

A stirred suspension of 5-bromopyridin-3-amine (100 mg, 0.493 mmol), (R)-4,6-dimethylpiperazin-2-one (70 mg, 0.546 mmol), copper iodide (14 mg, 0.074 mmol) and N,N′-dimethylethylenediamine (10 mg, 0.113 mmol) in dry 1,4-dioxane (2 mL) was heated at 100° C. for 16 h. The reaction mixture was filtered and concentrated in vacuo. Purification by column chromatography (12 g SiO₂, 0 to 30% methanol containing 5% ammonia in EtOAc) gave (R)-1-(5-aminopyridin-3-yl)-4,6-dimethylpiperazin-2-one (38 mg, 23%) as an oil. ¹H NMR (400 MHz, Chloroform-d) δ 8.01 (d, J=2.7 Hz, 1H), 7.87 (d, J=2.3 Hz, 1H), 6.85 (t, J=2.3 Hz, 1H), 4.02-3.94 (m, 2H), 3.84 (brs, 1H), 3.21 (m, 2H), 2.89 (dd, 1H), 2.50 (dd, 1H), 2.37 (s, 3H), 1.12 (dd, J=6.4, 2.3 Hz, 3H) ppm. ESI-MS m/z calc. 220.1324, found 221.14 (M+1)⁺; Retention time: 0.66 minutes.

### Intermediate CU

### 2-(morpholinomethyl)pyridin-4-amine

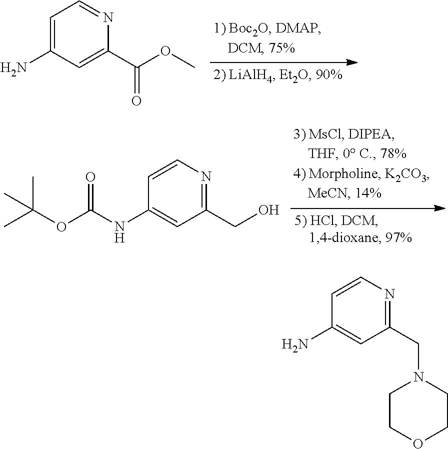 [SMILES: COC(=O)c1cc(N)ccn1 ; CC(C)(C)OC(=O)Nc1ccnc(CO)c1 ; Nc1ccnc(CN2CCOCC2)c1]

### Step 1:

Boc₂O (3.16 g, 14.479 mmol) and DMAP (160 mg, 1.310 mmol) were successively added to a solution of methyl 4-aminopicolinate (2 g, 13.145 mmol) in DCM (20 mL). The reaction mixture was stirred at ambient temperature for 16 h. The mixture was washed with water (20 mL), and the aqueous phase was extracted with DCM (20 mL). The combined organic extracts were dried (MgSO₄), filtered and concentrated in vacuo. Purification by flash chromatography (SiO₂, 50% EtOAc in heptane) gave methyl 4-((tert-butoxycarbonyl)amino)picolinate (2.51 g, 75%) as a white solid. ¹H NMR (400 MHz, Chloroform-d) δ 8.55 (d, J=5.5 Hz, 1H), 8.04 (d, J=2.3 Hz, 1H), 7.73 (q, J=2.6 Hz, 1H), 7.13 (s, 1H), 3.99 (s, 3H), 1.52 (s, 9H) ppm. ESI-MS m/z calc. 252.111, found 251.01 (M−1)⁻; Retention time: 0.71 minutes.

### Step 2:

A solution of LiAlH₄ (6.3 mL, 1 M in THF, 6.3 mmol) was added over 10 min to a solution of methyl 4-((tert-butoxycarbonyl)amino)picolinate (795 mg, 3.14 mmol) in diethyl ether (30 mL) under a nitrogen atmosphere. The reaction mixture was stirred for 2 h. Water (1 mL) and 2 N NaOH (0.24 mL) were carefully added and the reaction mixture was stirred for 30 min. Na₂SO₄ was added and the mixture was stirred for 30 min before filtering through a pad of Celite. The filtrates were concentrated in vacuo to give tert-butyl (2-(hydroxymethyl)pyridin-4-yl)carbamate (649 mg, 90%). H NMR (300 MHz, Chloroform-d) δ 8.36 (d, J=5.4 Hz, 1H), 7.38-7.32 (m, 1H), 7.13 (dd, J=5.6 Hz, 2.1 Hz, 1H), 6.78 (br s, 1H), 4.69 (s, 2H), 1.52 (s, 9H) ppm; OH alcohol not observed. ESI-MS m/z calc. 224.1161, found 225.2 (M+1)⁺; Retention time: 1.38 minutes.

### Step 3:

Mesyl chloride (444 mg, 0.3 mL, 3.87 mmol) was added over 5 min to a solution of tert-butyl (2-(hydroxymethyl)pyridin-4-yl)carbamate (649 mg, 2.81 mmol) and DIPEA (1.11 g, 1.5 mL, 8.61 mmol) in THF (5 mL) at 0° C. The reaction mixture was stirred at 0° C. for 2 h. A saturated aqueous NaHCO₃ solution (50 mL) was added and the mixture was extracted with EtOAc (2×50 mL). The combined organic extracts were dried (MgSO₄), filtered and concentrated in vacuo to afford (4-((tert-butoxycarbonyl)amino)pyridin-2-yl)methyl methanesulfonate (887 mg, 78%). ¹H NMR (300 MHz, Chloroform-d) δ 8.42 (d, J=5.6 Hz, 1H), 7.45-7.41 (m, 1H), 7.38-7.32 (m, 1H), 6.76 (br s, 1H), 5.26 (s, 2H), 3.08 (s, 3H), 1.52 (s, 9H) ppm. ESI-MS m/z calc. 302.09, found 303.1 (M+1)⁺; Retention time: 1.57 minutes.

### Step 4:

Morpholine (2.99 g, 3 mL, 34.4 mmol) and K₂CO₃ (6.7 g, 48.47 mmol) were successively added to a solution of (4-((tert-butoxycarbonyl)amino)pyridin-2-yl)methyl methanesulfonate (5 g, 16.537 mmol) in MeCN (90 mL). The mixture was stirred at ambient temperature for 18 h. Water (500 mL) was added and the mixture was extracted with EtOAc (3×200 mL). The combined organic extracts were dried (Na₂SO₄), filtered and concentrated in vacuo. Purification by flash chromatography (40 g SiO₂, 50 to 100% EtOAc in heptane) gave tert-butyl (2-(morpholinomethyl)pyridin-4-yl)carbamate (706 mg, 14%) as a yellow solid. ¹H NMR (300 MHz, Chloroform-d) δ 8.38 (d, J=5.6 Hz, 1H), 7.36 (d, J=2.2 Hz, 1H), 7.27-7.22 (m, 1H), 6.67 (br s, 1H), 3.76-3.69 (m 4H), 3.59 (s, 2H), 2.56-2.46 (m, 4H), 1.52 (s, 9H) ppm. ESI-MS m/z calc. 293.1739, found 294.2 (M+1)⁺; Retention time: 1.47 minutes.

### Step 5:

HCl (8 mL, 4 M in 1,4-dioxane, 32 mmol) was added to a solution of tert-butyl (2-(morpholinomethyl)pyridin-4-yl)carbamate (706 mg, 2.334 mmol) in DCM (15 mL). The reaction mixture was stirred for 18 h. The mixture was concentrated in vacuo to give 2-(morpholinomethyl)pyridin-4-amine (Hydrochloride salt (×2)) (618 mg, 97%) as a brown gum. ¹H NMR (400 MHz, DMSO-d₆) δ 8.36 (br s, 2H), 8.15 (d, J=7.0 Hz, 1H), 7.03 (d, J=2.1 Hz, 1H), 6.82 (dd, J=7.0, 2.3 Hz, 1H), 4.42 (br s, 2H), 4.18-3.72 (m, 6H), 3.18 (br s, 4H) ppm. ESI-MS m/z calc. 193.1215, found 194.2 (M+1)⁺; Retention time: 0.31 minutes.

The following intermediates were made using a method similar to that described in Intermediate CU, except that in Step 4, 3,3-difluoropyrrolidine (Hydrochloride salt) and pyrrolidine were respectively used as the amine instead of morpholine:

| Intermediate | | | |
|---|---|---|---|
| No. | Compound Name | LC/MS | NMR (shifts in ppm) |
| CV | 2-[(3,3- | ESI-MS m/z calc. | ¹H NMR (300 MHz, DMSO-d₆) δ |
| | difluoropyrrolidin-1- | 213.1078, found | 13.44 (s, 1H), 8.15 (s, 2H), 8.03 |
| | yl)methyl]pyridin-4- | 214.2 (M + 1)⁺; | (d, J = 6.8 Hz, 1H), 6.81 (d, J = |
| | amine (Hydrochloride | Retention time: 0.65 | 2.3 Hz, 1H), 6.74 (dd, J = 6.9, 2.5 |
| | salt (×2)) | minutes | Hz, 1H), 5.40 (br s, 1H), 3.92 (s, |
| | | | 2H), 3.26-3.11 (m, 2H), 2.95 (t, |
| | | | J = 6.6 Hz, 2H), 2.35 (tt, J = |
| | | | 14.8, 7.1 Hz, 2H) ppm. |
| CW | 2-(pyrrolidin-1- | ESI-MS m/z calc. | ¹H NMR (300 MHz, DMSO-d₆) δ |
| | ylmethyl)pyridin-4- | 177.1266, found | 11.47 (br s, 1H), 8.33 (br s, 2H), |
| | amine (2 Hydrochloride | 178.2 (M + 1)⁺; | 8.16 (d, J = 7.0 Hz, 1H), 7.04 (d, |
| | salt) | Retention time: 0.25 | J = 2.3 Hz, 1H), 6.81 (dd, J = 7.0, |
| | | minutes | 2.3 Hz, 1H), 4.51 (s, 2H), 4.00 |
| | | | (br s, 1H), 3.57-3.33 (m, 2H), |
| | | | 3.28-3.06 (m, 2H), 2.10-1.86 (m, |
| | | | 4H) ppm. |

### Intermediate CX

### tert-butyl (S)-3-((4-aminopyridin-2-yl)methyl)morpholine-4-carboxylate

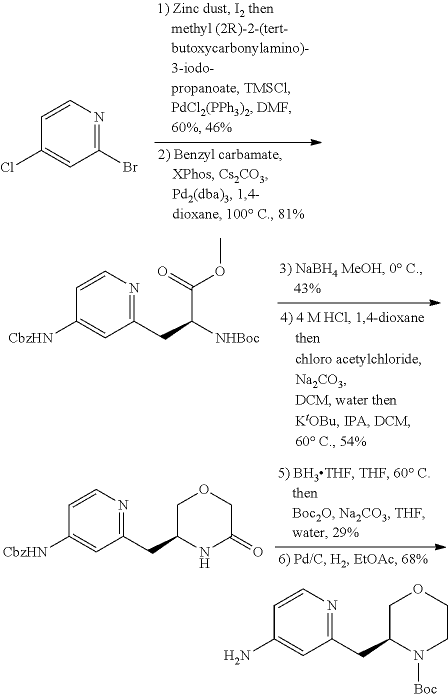 [SMILES: Clc1ccnc(Br)c1 ; COC(=O)[C@H](Cc1cc(NC(=O)OCc2ccccc2)ccn1)NC(=O)OC(C)(C)C ; O=C1COC[C@H](Cc2cc(NC(=O)OCc3ccccc3)ccn2)N1 ; CC(C)(C)OC(=O)N1CCOC[C@@H]1Cc1cc(N)ccn1]

### Step 1:

Iodine (1 g, 3.94 mmol) was added under nitrogen to a flask containing dried zinc dust (10 g, 152.93 mmol) (dried by heating under vacuo). The flask was then heated with heat gun while under high vacuum for 10 min. The mixture was cooled to 0° C. before addition of TMSCl (428 mg, 0.5 mL, 3.93 mmol). A solution of methyl (2R)-2-(tert-butoxycarbonylamino)-3-iodo-propanoate (8.6 g, 26.129 mmol) in degassed DMF (25 mL) was added dropwise to the mixture. The reaction was stirred at ambient temperature for 90 min. A solution of 2-bromo-4-chloro-pyridine (5 g, 25.982 mmol) and PdCl₂(PPh₃)₂(1.82 g, 2.59 mmol) in degassed DMF (25 mL) was then added and the mixture was heated at 60° C. for 2 h. The reaction mixture was quenched by addition of water (500 mL) and extracted with EtOAc (2×200 mL). The combined organic extracts were dried (MgSO₄) and concentrated in vacuo. Purification by flash chromatography (40 g SiO₂, 10 to 20% EtOAc in heptane) gave methyl (S)-2-((tert-butoxycarbonyl)amino)-3-(4-chloropyridin-2-yl)propanoate (3.8 g, 46%). ¹H NMR (400 MHz, DMSO-d₆) δ 8.47 (d, J=5.0 Hz, 1H), 7.46-7.38 (m, 2H), 7.29 (d, J=8.16 Hz, 1H), 4.48-4.43 (m, 1H), 3.61 (s, 3H), 3.17-3.12 (m, 1H), 3.05-2.99 (m, 1H), 1.31 (s, 9H) ppm. ESI-MS m/z calc. 314.1033, found 315.31 (M+1)⁺; Retention time: 1.78 minutes.

### Step 2:

A mixture of benzyl carbamate (870 mg, 5.755 mmol), methyl (S)-2-((tert-butoxycarbonyl)amino)-3-(4-chloropyridin-2-yl)propanoate (1.5 g, 4.765 mmol) and Cs₂CO₃ (3.1 g, 9.514 mmol) in 1,4-dioxane was degassed for 30 min with nitrogen. XPhos (340 mg, 0.7132 mmol) and Pd₂(dba)₃ (440 mg, 0.481 mmol) were successively added under a nitrogen atmosphere. The reaction mixture was heated at 100° C. for 3 h. The mixture was filtered through a pad of Celite and concentrated in vacuo. Purification by flash chromatography (SiO₂, 50 to 70% EtOAc in hexanes) gave methyl (S)-3-(4-(((benzyloxy)carbonyl)amino)pyridin-2-yl)-2-((tert-butoxycarbonyl)amino)propanoate (1.7 g, 81%) as a yellow solid. ¹H NMR (400 MHz, DMSO-d₆) δ 10.21 (s, 1H), 8.29 (d, J=5.52 Hz, 1H), 7.44-7.33 (m, 6H), 7.28 (d, J=5.48 Hz, 1H), 7.21 (d, J=8.12 Hz, 1H), 5.18 (s, 2H), 4.48-4.42 (m, 1H), 3.57 (d, J=7.76 Hz, 3H), 3.06-3.01 (m, 1H), 2.96-2.91 (m, 1H), 1.32 (s, 9H) ppm. ESI-MS m/z calc. 429.19, found 430.3 (M+1)⁺; Retention time: 1.98 minutes.

### Step 3:

NaBH₄ (800 mg, 21.146 mmol) was added portionwise to a solution of methyl (S)-3-(4-(((benzyloxy)carbonyl)amino)pyridin-2-yl)-2-((tert-butoxycarbonyl)amino)propanoate (3 g, 6.98 mmol) in MeOH (30 mL) at 0° C. The reaction mixture was stirred at ambient temperature for 18 h. Water (50 mL) was added, and the mixture was extracted with EtOAc (2×50 mL). The combined organic extracts were dried (MgSO₄), filtered and concentrated in vacuo. Purification by flash chromatography (40 g SiO₂, 50 to 70% EtOAc in heptane) gave tert-butyl (S)-(1-(4-(((benzyloxy)carbonyl)amino)pyridin-2-yl)-3-hydroxypropan-2-yl)carbamate (1.2 g, 43%). ESI-MS m/z calc. 401.1951, found 402.43 (M+1)⁺; Retention time: 1.55 minutes.

### Step 4:

HCl (5 mL, 4 M in 1,4-dioxane, 20 mmol) was added to a solution of tert-butyl (S)-(1-(4-(((benzyloxy)carbonyl)amino)pyridin-2-yl)-3-hydroxypropan-2-yl)carbamate (1.1 g, 2.91 mmol) in MeOH (20 mL). The reaction mixture was stirred overnight at ambient temperature and concentrated in vacuo. Na₂CO₃ (310 mg, 2.92 mmol) and chloro acetylchloride (0.3 mL, 3.77 mmol) were successively added to a solution of the residue in mixture of DCM (20 mL) and water (20 mL). The reaction mixture was stirred at ambient temperature for 1 h, The mixture was diluted with water (100 mL) and extracted with DCM (2×50 mL). The combined organic extracts were dried (MgSO₄), filtered and concentrated in vacuo. K^{t}OBu (980 mg, 8.733 mmol) was added to a solution of the residue in a mixture of IPA (10 mL) and DCM (10 mL). The reaction mixture was heated at 60° C. for 2 h. The mixture was concentrated in vacuo, diluted with water (50 mL) and extracted with DCM (3×30 mL). The combined organic extracts were dried (MgSO₄), filtered and concentrated in vacuo. Purification by flash chromatography (40 g SiO₂, 2% MeOH in DCM) gave benzyl (S)-(2-((5-oxomorpholin-3-yl)methyl)pyridin-4-yl)carbamate (550 mg, 54%). ESI-MS m/z calc. 341.1376, found 342.39 (M+1)⁺; Retention time: 1.41 minutes.

### Step 5:

BH₃·THF (9 mL, 1 M solution in THF, 9 mmol) was added to a stirred solution of benzyl (S)-(2-((5-oxomorpholin-3-yl)methyl)pyridin-4-yl)carbamate (500 mg, 1.46 mmol) in THF (20 mL). The reaction mixture was heated at 60° C. for 2 h before quenching with MeOH (5 mL) and heating again at 70° C. overnight. The reaction mixture was concentrated in vacuo. Na₂CO₃ (470 mg, 4.435 mmol) and Boc₂0 (475 mg, 0.5 mL, 2.176 mmol) were successively added ta a mixture of the residue dissolved in a DCM (10 mL) and water (10 mL). The reaction mixture was stirred for 2 h. The mixture was diluted with water (50 mL) and extracted with DCM (2×50 mL). The combined organic extracts were dried (MgSO₄), filtered and concentrated in vacuo. Purification by flash chromatography (SiO₂, 30% to 50% EtOAc in hexanes) gave tert-butyl (S)-3-((4-(((benzyloxy)carbonyl)amino)pyridin-2-yl)methyl)morpholine-4-carboxylate (180 mg, 29%). ESI-MS m/z calc. 427.2107, found 428.47 (M+1)⁺; Retention time: 1.52 minutes.

### Step 6:

Pd/C (50 mg, 20% w/w, 0.094 mmol) was added to a degassed solution of tert-butyl (S)-3-((4-(((benzyloxy)carbonyl)amino)pyridin-2-yl)methyl)morpholine-4-carboxylate (160 mg, 0.374 mmol) in EtOAc (5 mL). The reaction mixture was stirred under a hydrogen atmosphere at ambient temperature for 18 h. The mixture was filtered through a pad of Celite and concentrated in vacuo to give tert-butyl (S)-3-((4-aminopyridin-2-yl)methyl)morpholine-4-carboxylate (102 mg, 68%) as a white sticky solid. ESI-MS m/z calc. 293.1739, found 294.21 (M+1)⁺; Retention time: 1.71 minutes.

The following intermediate was made using a method similar to that described in Intermediate CX, except that Step 2 was carried out between Steps 5 and 6:

| Intermediate | | | |
|---|---|---|---|
| No. | Compound Name | LC/MS | NMR (shifts in ppm) |
| CY | tert-butyl (R)-3-((4- | ESI-MS m/z calc. | ¹H NMR (400 MHz, DMSO-d₆) δ |
| | aminopyridin-2- | 293.1739, found | 7.88 (d, J = 5.56 Hz, 1H), 6.31 (s, |
| | yl)methyl)morpholine- | 294.21 (M + 1)⁺; | 2H), 5.92 (s, 2H), 4.16-4.13 (m, |
| | 4-carboxylate | Retention time: 1.72 | 1H), 3.82-3.80 (m, 1H), 3.65-3.62 |
| | | minutes | (m, 2H), 3.40-3.37 (m, 2H), |
| | | | 3.20-3.14(m, 1H), 2.80-2.66(m, |
| | | | 2H), 1.27(s, 9H) ppm. |

### Intermediate CZ

### tert-butyl 4-((5-aminopyridin-2-yl)methyl)piperidine-1-carboxylate

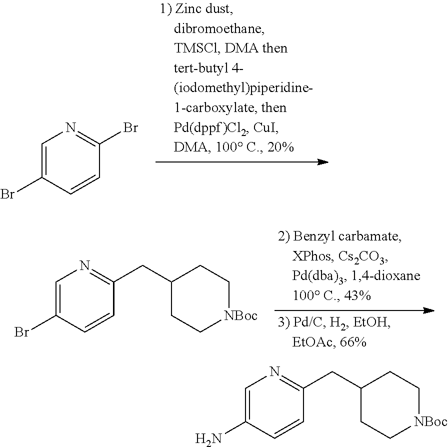 [SMILES: Brc1ccc(Br)nc1 ; CC(C)(C)OC(=O)N1CCC(Cc2ccc(Br)cn2)CC1 ; CC(C)(C)OC(=O)N1CCC(Cc2ccc(N)cn2)CC1]

### Step 1:

TMSCl (171.20 mg, 0.2 mL, 1.576 mmol) and 1,2-dibromoethane (436 mg, 0.2 mL, 2.321 mmol) were added to a solution of activated Zinc powder (1 g, 15.293 mmol) in DMA (4 mL). The reaction mixture was stirred at ambient temperature for 10 min under an argon atmosphere. A solution of tert-butyl 4-(iodomethyl)piperidine-1-carboxylate (3.2 g, 9.841 mmol) in DMA (10 mL) was added and the reaction mixture was stirred at ambient temperature for 1 h. The resulting mixture was then added into a degassed mixture of 2,5-dibromopyridine (1.2 g, 5.066 mmol), Pd(dppf)Cl₂ (360 mg, 0.492 mmol) and CuI (100 mg, 0.525 mmol) in DMA (10 mL). The reaction was heated at 100° C. for 2 h. The mixture was filtered through a pad of Celite, washing with EtOAc. The filtrates were collected and washed with brine and water, dried (Na₂SO₄) and concentrated in vacuo. Purification by flash chromatography (40 g SiO₂, 10 to 15% EtOAc in hexanes) gave tert-butyl 4-((5-bromopyridin-2-yl)methyl)piperidine-1-carboxylate (700 mg, 20%). ¹H NMR (400 MHz, DMSO-d₆) δ 8.60 (d, J=2.12 Hz, 1H), 7.95-7.92 (m, 1H), 7.24 (d, J=8.2 Hz, 1H), 3.90-3.87 (m, 2H), 2.64-2.62 (m, 4H), 1.87-1.86 (m, 1H), 1.50 (d, J=12.84 Hz, 2H), 1.38 (s, 9H), 1.08-1.00 (m, 2H) ppm. ESI-MS m/z calc. 354.0943, found 355.1 (M+1)⁺; Retention time: 3.17 minutes.

### Step 2:

Benzyl carbamate (460 mg, 3.043 mmol), Cs₂CO₃ (1.6 g, 4.91 mmol), Pd₂(dba)₃ (185 mg, 0.202 mmol) and XPhos (190 mg, 0.398 mmol) were successively added to a solution of tert-butyl 4-((5-bromopyridin-2-yl)methyl)piperidine-1-carboxylate (700 mg, 1.97 mmol) in 1,4-dioxane (20 mL) under a nitrogen atmosphere. The reaction mixture was heated at 100° C. overnight. The mixture was filtered through a pad of Celite. The mixture was partitioned between EtOAc (250 mL) and water (80 mL). The organic phase was separated and concentrated in vacuo. Purification by flash chromatography (SiO₂, 45 to 50% EtOAc in hexanes) gave tert-butyl 4-((5-(((benzyloxy)carbonyl)amino)pyridin-2-yl)methyl)piperidine-1-carboxylate (360 mg, 43%) as a light yellow solid. ¹H NMR (400 MHz, DMSO-d₆) δ 9.88 (s, 1H), 8.53 (s, 1H), 7.77 (d, J=6.8 Hz, 1H), 7.41-7.34 (m, 5H), 7.15 (d, J=8.48 Hz, 1H), 5.16 (s, 2H), 3.90-3.86 (m, 2H), 2.63-2.56 (m, 4H), 1.84-1.83 (m, 1H), 1.52-1.45 (m, 2H), 1.37 (s, 9H), 0.88-0.81 (m, 2H) ppm. ESI-MS m/z calc. 425.2315, found 426.3 (M+1)⁺; Retention time: 3.04 minutes.

### Step 3:

Pd/C (175 mg, 10% w/w, 0.164 mmol) was added to a degassed solution of tert-butyl 4-((5-(((benzyloxy)carbonyl)amino)pyridin-2-yl)methyl)piperidine-1-carboxylate (350 mg, 0.822 mmol) in a mixture of EtOH (8 mL) and EtOAc (8 mL). The reaction mixture was stirred under a hydrogen atmosphere at ambient temperature for 18 h. The mixture was filtered through a pad of Celite. The filtrates were collected and concentrated in vacuo. Purification by flash chromatography (SiO₂, 25 to 30% EtOAc in hexanes) gave tert-butyl 4-((5-aminopyridin-2-yl)methyl)piperidine-1-carboxylate (160 mg, 66%). ¹H NMR (400 MHz, DMSO-d₆) δ 7.84 (s, 1H), 6.83 (s, 2H), 5.03 (s, 2H), 3.87 (d, J=10.6 Hz, 2H), 2.69-2.62 (m, 2H), 2.44-2.42 (m, 2H), 1.77 (s, 1H), 1.51-1.48 (m, 2H), 1.37 (s, 9H), 1.08-0.86 (m, 2H) ppm. ESI-MS m/z calc. 291.1947, found 292.28 (M+1)⁺; Retention time: 1.47 minutes.

### Intermediate DA

### tert-butyl (1-(6-aminopyrimidin-4-yl)-2-methoxyethyl)carbamate

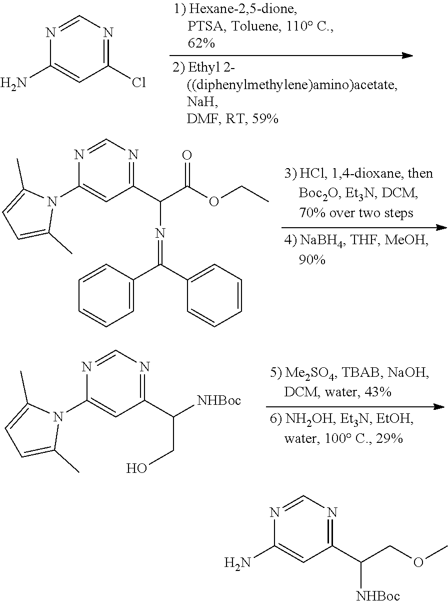

### Step 1:

Hexane-2,5-dione (18 g, 18.5 mL, 157.7 mmol) and PTSA (1.5 g, 7.88 mmol) were successively added to a solution of 6-chloropyrimidin-4-amine (10 g, 77.19 mmol) in toluene (200 mL). The reaction mixture, flanked with a Dean-Stark apparatus, was heated to reflux overnight and concentrated in vacuo. The material was dissolved with EtOAc (500 mL) and washed with water (200 mL). The organic layer was separated, dried (Na₂SO₄) and concentrated in vacuo. Purification by flash chromatography (40 g SiO₂, 10 to 15% EtOAc in hexanes) gave 4-chloro-6-(2,5-dimethyl-1H-pyrrol-1-yl)pyrimidine (10 g, 62%). ¹H NMR (400 MHz, DMSO-d₆) δ 9.05 (s, 1H),7.83 (s, 1H), 5.91 (s, 2H), 2.21 (s, 6H) ppm. ESI-MS m/z calc. 207.05, found 208.31 (M+1); Retention time: 1.894 minutes.

### Step 2:

NaH (3.89 g, 60% w/w, 97.26 mmol) was added to a solution of ethyl 2-((diphenylmethylene)amino)acetate (13 g, 48.63 mmol) in DMF (210 mL) at 0° C. The reaction mixture was stirred at ambient temperature for 30 min. A solution of 4-chloro-6-(2,5-dimethyl-1H-pyrrol-1-yl)pyrimidine (10 g, 48.15 mmol) in DMF (30 mL) was added dropwise. The mixture was stirred at ambient temperature for 1 h and partitioned between EtOAc (500 mL) and water (200 mL). The organic extract was separated, dried (MgSO₄) and concentrated in vacuo. Purification by flash chromatography (80 g SiO₂, 30% EtOAc in hexanes) gave ethyl 2-(6-(2,5-dimethyl-1H-pyrrol-1-yl)pyrimidin-4-yl)-2-((diphenylmethylene)amino)acetate (12.5 g, 59%). ¹H NMR (400 MHz, DMSO-d₆) δ 9.10 (s, 1H), 7.68 (s, 1H), 7.64-7.62 (m, 2H), 7.56-7.55 (m, 3H), 7.50-7.48 (m, 1H), 7.45-7.41 (m, 2H), 7.23-7.21 (m, 2H), 5.92 (s, 2H), 5.25 (s, 1H), 4.10 (q, J=7.0 Hz, 2H), 2.21 (s, 6H), 1.12 (t, J=7.0 Hz, 3H) ppm. ESI-MS m/z calc. 438.20, found 439.42 (M+1)⁺; Retention time: 2.03 minutes.

### Step 3:

4 M HCl (14.5 mL, solution in 1,4-dioxane, 58 mmol) was added to a solution of ethyl 2-(6-(2,5-dimethyl-1H-pyrrol-1-yl)pyrimidin-4-yl)-2-((diphenylmethylene)amino)acetate (12.5 g, 28.5 mmol) in 1,4-dioxane (250 mL) at 0° C. The reaction mixture was stirred at ambient temperature for 1 h and concentrated in vacuo. Et₃N (14 mL, 100.44 mmol) and Boc₂0 (12.35 g, 56.587 mmol) were successively added to a solution of the residue in DCM (200 mL) at 0° C. The reaction mixture was stirred at ambient temperature for 4 h. The mixture was washed with water (200 mL). The aqueous phase was extracted with DCM (400 mL). The combined organic extracts were concentrated in vacuo. Purification by flash chromatography (80 g SiO₂, 10% EtOAc in hexanes) gave ethyl 2-((tert-butoxycarbonyl)amino)-2-(6-(2,5-dimethyl-1H-pyrrol-1-yl)pyrimidin-4-yl)acetate (7.5 g, 70%). ¹H NMR (400 MHz, DMSO-d₆) δ 9.14 (s, 1H), 7.83-7.81 (m, 1H), 7.61 (s, 1H), 5.91 (s, 2H), 5.45 (d, J=8.4 Hz, 1H), 4.17-4.12 (m, 2H), 2.18 (s, 6H), 1.37 (s, 9H), 1.15 (t, J=7.08 Hz, 3H) ppm. ESI-MS m/z calc. 374.1954, found 375.41 (M+1)⁺; Retention time: 2.02 minutes.

### Step 4:

NaBH₄ (760 mg, 20.089 mmol) was added portionwise to a solution of ethyl 2-((tert-butoxycarbonyl)amino)-2-(6-(2,5-dimethyl-1H-pyrrol-1-yl)pyrimidin-4-yl)acetate (7.5 g, 20.03 mmol) in THF (100 mL) and MeOH (20 mL) at 0° C. The reaction mixture was stirred at ambient temperature for 4 h. The mixture was concentrated in vacuo and partitioned between EtOAc (300 mL) and water (100 mL). The organic extract was separated and concentrated in vacuo. Purification by flash chromatography (80 g SiO₂, 40% EtOAc in hexanes) gave tert-butyl (1-(6-(2,5-dimethyl-1H-pyrrol-1-yl)pyrimidin-4-yl)-2-hydroxyethyl)carbamate (6 g, 90%). ¹H NMR (400 MHz, DMSO-d₆) δ 9.11 (s, 1H), 7.40 (s, 1H), 7.32 (d, J=8 Hz, 1H), 5.89 (s, 2H), 4.91 (t, J=5.96 Hz, 1H), 4.66-4.64 (m, 1H), 3.75-3.66 (m, 2H), 2.16 (s, 6H), 1.37 (s, 9H) ppm. ESI-MS m/z calc. 332.1848, found 333.45 (M+1)⁺; Retention time: 1.69 minutes.

### Step 5:

TBAB (400 mg, 1.241 mmol) and 1 M NaOH (1.5 mL, 1.5 mmol) were successively added to a stirred solution of tert-butyl (1-(6-(2,5-dimethyl-1H-pyrrol-1-yl)pyrimidin-4-yl)-2-hydroxyethyl)carbamate (140 mg, 0.406 mmol) in DCM (5 mL). The reaction mixture was stirred for 10 min. Me₂SO₄ (332.5 mg, 0.25 mL, 2.63 mmol) was added and the reaction mixture was stirred at ambient temperature for 16 h. The mixture was partitioned between DCM (20 mL) and water (10 mL). The organic extracts were separated and concentrated in vacuo. Purification by flash chromatography (SiO₂, 10% EtOAc in hexanes) gave tert-butyl (1-(6-(2,5-dimethyl-1H-pyrrol-1-yl)pyrimidin-4-yl)-2-methoxyethyl)carbamate (60 mg, 43%). ¹H NMR (400 MHz, DMSO-d₆) δ 9.12 (s, 1H), 7.52 (d, J=7.48 Hz, 1H), 7.43 (s, 1H), 5.90 (s, 2H), 4.82-4.81 (m, 1H), 3.63-3.61 (m, 2H), 3.25 (s, 3H), 2.21 (s, 6H), 1.37 (s, 9H) ppm. ESI-MS m/z calc. 346.2005, found 347.41 (M+1)⁺; Retention time: 1.939 minutes.

### Step 6:

Et₃N (544.5 mg, 0.75 mL, 5.381 mmol) and NH₂OH·HCl (2 g, 28.781 mmol) were successively added to a solution of tert-butyl (1-(6-(2,5-dimethyl-1H-pyrrol-1-yl)pyrimidin-4-yl)-2-methoxyethyl)carbamate (600 mg, 1.73 mmol) in a mixture of EtOH (15 mL) and H₂O (3.75 mL). The reaction mixture was heated at 100° C. for 16 h and then quenched by pouring into an ice-cold 1 M HCl solution (20 mL). The mixture was extracted with EtOAc (50 mL). The pH of the aqueous layer was adjusted to pH 9-10 by the addition of 6 M NaOH and extracted with EtOAc (100 mL). The organic extract was dried (Na₂SO₄) and concentrated in vacuo to give tert-butyl (1-(6-aminopyrimidin-4-yl)-2-methoxyethyl)carbamate (148 mg, 29%). ¹H NMR (400 MHz, DMSO-d₆) δ 8.25 (s, 1H), 7.21 (d, J=7.72 Hz, 1H), 6.85 (s, 2H), 6.36 (s, 1H), 4.48-4.47 (m, 1H), 3.58-3.54 (m, 1H), 3.49-3.40 (m, 1H), 3.23 (s, 3H), 1.38 (s, 9H) ppm. ESI-MS m/z calc. 268.1535, found 269.2 (M+1)⁺; Retention time: 1.698 minutes.

### Intermediate DB

### 6-(2-((tert-butyldimethylsilyl)oxy)propan-2-yl)pyridazin-4-amine

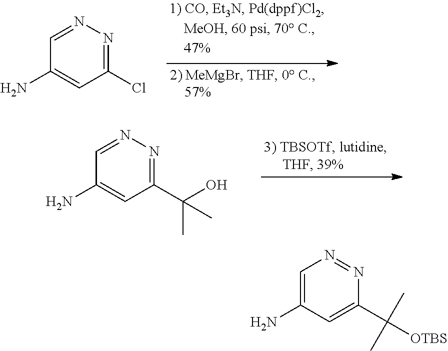 [SMILES: Nc1cnnc(Cl)c1 ; CC(C)(O)c1cc(N)cnn1 ; CC(C)(O[Si](C)(C)C(C)(C)C)c1cc(N)cnn1]

### Step 1:

In a stainless-steel pressure vessel, Et₃N (159.72 g, 220 mL, 1.578 mol) and Pd(dppf)Cl₂ (26 g, 35.533 mmol) were successively added to a solution of 6-chloropyridazin-4-amine (50 g, 385.96 mmol) in MeOH (750 mL). A CO cylinder was fitted. The reaction mixture was purged three times with carbon monoxide and the CO gas pressure was set to 60 psi. The mixture was heated at 70° C. for 60 h. The reaction mixture was filtered through a pad of Celite, washing with MeOH. The filtrate was concentrated in vacuo. Purification by flash chromatography (3×330 g SiO₂, 10% MeOH containing 3% NH₄OH in DCM) gave methyl 5-aminopyridazine-3-carboxylate (46 g, 47%). ¹H NMR (400 MHz, DMSO-d₆) δ 8.67-8.63 (m, 1H), 7.20-7.16 (m, 1H), 6.80 (s, 2H), 3.88 (s, 3H) ppm. ESI-MS m/z calc. 153.0538, found 154.4 (M+1)⁺; Retention time: 0.81 minutes.

### Step 2:

A suspension of methyl 5-aminopyridazine-3-carboxylate (500 mg, 1.633 mmol) in THF (40 mL) was sonicated for 45 minutes under nitrogen and then transferred onto a 3 M solution of MeMgBr (11 mL, solution in Et₂O, 33 mmol) at 0° C. The reaction mixture was stirred at 0° C. for 30 min and then at ambient temperature for 1.5 h. The mixture was quenched at 0° C. by slow addition of a saturated aqueous NaHCO₃ solution (10 mL) (exothermic and vigorous gas evolution). The reaction mixture was decanted from the solids and the solids were further washed with EtOAc (2×25 mL). The combined organic extracts were filtered and concentrated in vacuo to give 2-(5-aminopyridazin-3-yl)propan-2-ol (357 mg, 57%) as an orange crystalline solid. ¹H NMR (250 MHz, Methanol-d₄) δ 8.39 (d, J=2.8 Hz, 1H), 7.02 (d, J=2.6 Hz, 1H), 1.55 (s, 6H) ppm; NH₂ amine and OH alcohol not observed.

### Step 3:

2,6-Lutidine (0.117 mL, 1.01 mmol) and TBSOTf (253 mg, 0.22 mL, 0.95 mmol) were successively added to a suspension of 2-(5-aminopyridazin-3-yl)propan-2-ol (100 mg, 0.43 mmol) in THF (1 mL) at 0° C. The reaction mixture was stirred at 0° C. for 10 min then at ambient temperature for 2 h. The mixture was concentrated in vacuo, diluted with MTBE (5 mL) and washed with water (4 mL). The organic extracts were dried (Na₂SO₄), filtered and concentrated in vacuo. Purification by flash chromatography (12 g SiO₂, 2.5% MeOH containing 0.05 M NH₃ in DCM) gave 6-[1-[tert-butyl(dimethyl)silyl]oxy-1-methyl-ethyl]pyridazin-4-amine (48.7 mg, 39%) as an off white solid. ¹H NMR (400 MHz, DMSO-d₆) δ 8.37 (d, J=2.7 Hz, 1H), 6.79 (d, J=2.7 Hz, 1H), 6.33 (s, 2H), 1.53 (s, 6H), 0.90 (s, 9H), 0.07 (s, 6H) ppm. ESI-MS m/z calc. 267.1767, found 268.2 (M+1)⁺; Retention time: 1.98 minutes.

### Intermediate DC

### 2-(3-((tert-butyldimethylsilyl)oxy)tetrahydrofuran-3-yl)pyridin-4-amine

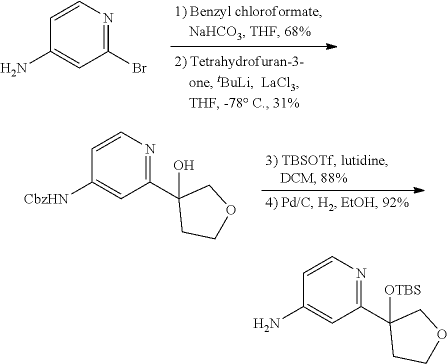 [SMILES: Nc1ccnc(Br)c1 ; O=C(Nc1ccnc(C2(O)CCOC2)c1)OCc1ccccc1 ; CC(C)(C)[Si](C)(C)OC1(c2cc(N)ccn2)CCOC1]

### Step 1:

Benzyl chloroformate (1.2 g, 1 mL, 50% w/w, 3.51 mmol) was added over 5 min to a solution of the 2-bromopyridin-4-amine (500 mg, 2.89 mmol) and NaHCO₃ (490 mg, 5.83 mmol) in water (10 mL) and acetone (25 mL) at 0° C. The reaction mixture was stirred at 0° C. for 1 h, and then at ambient temperature overnight. The mixture was concentrated in vacuo and the residue was partitioned between water and EtOAc. The organic extract was separated and washed with a saturated aqueous NaHCO₃ solution and brine, dried (Na₂SO₄) and concentrated in vacuo. Purification by flash chromatography (12 g SiO₂, 10% EtOAc in hexanes) gave benzyl (2-bromopyridin-4-yl)carbamate (600 mg, 68%). ¹H NMR (400 MHz, DMSO-d₆) δ 10.49 (s, 1H), 8.19 (d, J=5.7 Hz, 1H), 7.70 (d, J=1.9 Hz, 1H), 7.47-7.28 (m, 6H), 5.19 (s, 2H) ppm. ESI-MS m/z calc. 306.00, found 307.0 (M+1)⁺; Retention time: 1.88 minutes.

### Step 2:

A solution of tetrahydrofuran-3-one (2.3 g, 26.71 mmol) in lanthanum trichloride lithium chloride complex (39 mL, 0.6 M in THF, 23.4 mmol) was stirred under nitrogen for 1 h. In a separate flask, ^{t}BuLi (41.5 mL, 1.7 M in pentanes, 70.55 mmol) was slowly added to a solution of benzyl (2-bromopyridin-4-yl)carbamate (7.2 g, 23.442 mmol) in THF (432 mL) at −78° C. The reaction mixture was stirred at −78° C. for 15 min. The prepared ketone-LaCl₃·2LiCl solution in THF was then added via cannula. The reaction mixture was stirred at −78° C. for 15 min and then allowed to warm to ambient temperature. The mixture was cooled to 0° C. before addition of a NaHCO₃ solution (125 mL). The mixture was extracted with EtOAc (3×250 mL). The combined organic extracts were dried (Na₂SO₄), filtered and concentrated in vacuo. Purification by flash chromatography (SiO₂, 10% EtOAc in hexanes) gave benzyl (2-(3-hydroxytetrahydrofuran-3-yl)pyridin-4-yl)carbamate (4.52 g, 31%). ESI-MS m/z calc. 314.1267, found 315.4 (M+1)⁺; Retention time: 2.96 minutes.

### Step 3:

2,6-Lutidine (5 mL, 42.929 mmol) and TBDMSOTf (6 mL, 26.103 mmol) were successively added to a solution of benzyl (2-(3-hydroxytetrahydrofuran-3-yl)pyridin-4-yl)carbamate (4.52 g, 7.190 mmol) in DCM (45 mL) at 0° C. The reaction mixture was stirred at ambient temperature for 1 h. The mixture was cooled to 0° C. and a solution of NaHCO₃ (125 mL) was added. The mixture was extracted with EtOAc (3×250 mL). The combined organic extracts were dried (Na₂SO₄), filtered and concentrated in vacuo. Purification by flash chromatography (SiO₂, 0 to 40% EtOAc in hexanes) gave benzyl (2-(3-((tert-butyldimethylsilyl)oxy)tetrahydrofuran-3-yl)pyridin-4-yl)carbamate (2.74 g, 88%) as a colourless oil. ¹H NMR (400 MHz, Chloroform-d) δ 8.41 (d, J=6 Hz, 1H), 7.54 (d, J=2.0 Hz, 1H), 7.46-7.33 (m, 6H), 6.81 (s, 1H), 5.23 (s, 2H), 4.15-3.93 (m, 4H), 2.69-2.51 (m, 1H), 2.31-2.13 (m, 1H), 0.94 (s, 9H), 0.14-−0.10 (m, 6H) ppm. ESI-MS m/z calc. 428.2131, found 429.3 (M+1)⁺; Retention time: 5.52 minutes.

### Step 4:

Pd-C(680.1 mg, 10% w/w, 0.639 mmol) was added to a solution of benzyl (2-(3-((tert-butyldimethylsilyl)oxy)tetrahydrofuran-3-yl)pyridin-4-yl)carbamate (2.74 g, 6.39 mmol) in EtOH (45 mL) under a nitrogen atmosphere. The reaction mixture was stirred under a hydrogen atmosphere at ambient temperature for 14 h, filtered through a Celite pad and concentrated in vacuo to give 2-(3-((tert-butyldimethylsilyl)oxy)tetrahydrofuran-3-yl)pyridin-4-amine (1.81 g, 92%) as a waxy white solid. ¹H NMR (400 MHz, DMSO-d₆) δ 7.93 (d, J=6 Hz, 1H), 6.68 (d, J=2 Hz, 1H), 6.37 (dd, J=6, 2 Hz, 1H), 6.02 (s, 2H), 4.01-3.87 (m, 3H), 3.87-3.82 (m, 1H), 2.48-2.39 (m, 1H), 2.13-2.02 (m, 1H), 0.87 (s, 9H), −0.07 (m, 6H) ppm. ESI-MS m/z calc. 294.1764, found 295.2 (M+1)⁺; Retention time: 1.84 minutes.

### Intermediate DD

### 6-(2-((tert-butyldimethylsilyl)oxy)-1-fluoropropan-2-yl)pyridin-3-amine

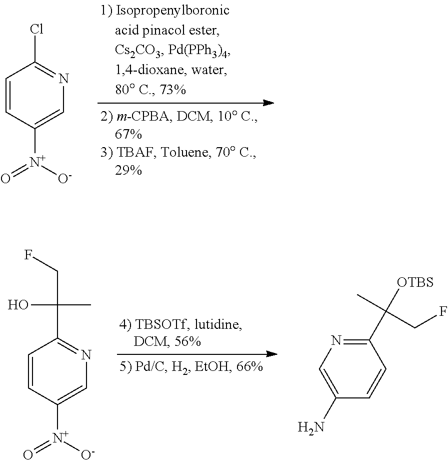 [SMILES: O=[N+]([O-])c1ccc(Cl)nc1 ; CC(O)(CF)c1ccc([N+](=O)[O-])cn1 ; CC(CF)(O[Si](C)(C)C(C)(C)C)c1ccc(N)cn1]

### Step 1:

Isopropenylboronic acid pinacol ester (6.5 g, 38.68 mmol), Pd(PPh₃)₄ (2 g, 1.73 mmol) and Cs₂CO₃ (30 g, 92.07 mmol) were successively added to a solution of 2-chloro-5-nitropyridine (5 g, 31.53 mmol) in 1,4-dioxane (40 mL) and water (4 mL). The reaction mixture was heated at 80° C. for 14 h and then filtered and concentrated in vacuo. Purification by flash chromatography (SiO₂, 5% EtOAc in hexanes) gave 5-nitro-2-(prop-1-en-2-yl)pyridine (3.8 g, 73%). ¹H NMR (400 MHz, DMSO-d₆) δ 9.34 (s, 1H), 8.56 (dd, J=8, 3 Hz, 1H), 7.92 (d, J=8 Hz, 1H), 6.17 (s, 1H), 5.59 (s, 1H), 2.20 (s, 3H) ppm. ESI-MS m/z calc. 164.0586, found 165.1 (M+1)⁺; Retention time: 2.41 minutes.

### Step 2:

m-CPBA (4 g, 23.18 mmol) was added portionwise to a solution of 5-nitro-2-(prop-1-en-2-yl)pyridine (2.5 g, 15.229 mmol) in DCM (50 mL) at 0° C. The reaction mixture was stirred at 5 to 10° C. for 9 h. A saturated NaHCO₃ solution was added and the mixture was extracted with DCM (2×100 mL). The combined organic extracts were dried (Na₂SO₄), filtered and concentrated in vacuo. Purification by flash chromatography (SiO₂, 20% EtOAc in hexanes) gave 2-(2-methyloxiran-2-yl)-5-nitropyridine (1.9 g, 67%). ¹H NMR (400 MHz, DMSO-d₆) δ 9.34 (s, 1H), 8.58-8.56 (m, 1H), 7.56 (d, J=8 Hz, 1H), 3.17 (s, 1H), 3.00 (s, 1H), 1.74 (s, 3H) ppm. ESI-MS m/z calc. 180.05, found 181.1 (M+1)⁺; Retention time: 1.98 minutes.

### Step 3:

1 M TBAF (10 mL, solution in THF, 10 mmol) was added to a solution of 2-(2-methyloxiran-2-yl)-5-nitropyridine (500 mg, 2.77 mmol) in toluene (10 mL). The reaction mixture was heated at 70° C. for 12 h. The mixture was concentrated in vacuo. Purification by flash chromatography (SiO₂, 20% EtOAc in hexanes) gave 1-fluoro-2-(5-nitropyridin-2-yl)propan-2-ol (160 mg, 29%). ¹H NMR (400 MHz, DMSO-d₆) δ 9.32 (s, 1H), 8.63-8.61 (m, 1H), 7.96 (d, J=8 Hz, 1H), 6.02 (s, 1H), 4.61 (t, J=48 Hz, 1H), 4.59 (t, J=48 Hz, 1H), 1.43 (s, 3H) ppm.

### Step 4:

TBSOTf (0.6 mL, 2.61 mmol) was added dropwise to a solution of 1-fluoro-2-(5-nitropyridin-2-yl)propan-2-ol (250 mg, 1.249 mmol) and 2,6-lutidine (0.6 mL, 5.181 mmol) in DCM (10 mL) at 0° C. The reaction mixture was stirred at ambient temperature for 18 h. The reaction mixture was quenched with water and extracted with DCM (2×50 mL). The combined organic extracts were dried (Na₂SO₄), filtered and concentrated in vacuo. Purification by flash chromatography (SiO₂, 5 to 10% EtOAc in hexanes) gave 2-(2-((tert-butyldimethylsilyl)oxy)-1-fluoropropan-2-yl)-5-nitropyridine (220 mg, 56%). ¹H NMR (400 MHz, DMSO-d₆) δ 9.34 (s, 1H), 8.70-8.67 (m, 1H), 7.91 (d, J=8 Hz, 1H), 4.78-4.54 (m, 2H), 1.55 (s, 3H), 0.95 (s, 9H), 0.13 (s, 6H) ppm.

### Step 5:

Pd/C (80 mg, 10% w/w, 0.075 mmol) was added to a solution of 2-(2-((tert-butyldimethylsilyl)oxy)-1-fluoropropan-2-yl)-5-nitropyridine (200 mg, 0.636 mmol) in EtOH (45 mL) under a nitrogen atmosphere. The reaction mixture was stirred under a hydrogen atmosphere at ambient temperature for 14 h, filtered through a Celite pad and concentrated in vacuo. Purification by flash chromatography (SiO₂, 20% EtOAc in hexanes) gave 6-(2-((tert-butyldimethylsilyl)oxy)-1-fluoropropan-2-yl)pyridin-3-amine (120 mg, 66%). ¹H NMR (400 MHz, DMSO-d₆) δ 7.85 (s, 1H), 7.27 (d, J=8 Hz, 1H), 6.93-6.91 (m, 1H), 5.26 (br s, 2H), 4.55-4.47 (m, 1H), 4.42-4.35 (m, 1H), 1.49 (s, 3H), 0.88 (s, 9H), 0.006 (s, 3H), −0.024 (s, 3H) ppm.

### Intermediate DE

### 2-(2-((tert-butyldimethylsilyl)oxy)-1-fluoropropan-2-yl)pyridin-4-amine

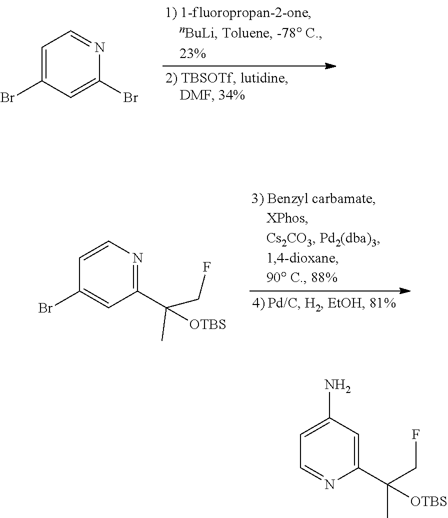 [SMILES: Brc1ccnc(Br)c1 ; CC(CF)(O[Si](C)(C)C(C)(C)C)c1cc(Br)ccn1 ; CC(CF)(O[Si](C)(C)C(C)(C)C)c1cc(N)ccn1]

### Step 1:

ⁿBuLi (7.59 mL, 2 M solution in hexanes, 15.197 mmol) was added to a solution of 2,4-dibromopyridine (3 g, 12.664 mmol) in toluene (25 mL) at −78° C. The mixture was stirred at −78° C. for 15 min. A solution of 1-fluoropropan-2-one (1.156 g, 1.1 mL, 15.19 mmol) in toluene (10 mL) was added and the mixture was stirred at −78° C. for 45 min. A saturated solution of NH₄Cl was added and the mixture was extracted with EtOAc (3×60 mL). The combined organic extracts were washed with water (1×40 mL), brine (1×40 mL), dried (Na₂SO₄) and concentrated in vacuo. Purification by flash chromatography (SiO₂, 20% EtOAc in hexanes) gave 2-(4-bromopyridin-2-yl)-1-fluoropropan-2-ol (700 mg, 23%). ¹H NMR (400 MHz, DMSO-d₆) δ 8.43 (d, J=5.2 Hz, 1H), 7.86 (d, J=1.8 Hz, 1H), 7.58 (dd, J=1.9 Hz, 5.28 Hz, 1H), 5.81 (s, 1H), 4.65-4.40 (m, 2H), 3.30 (d, J=2.2 Hz, 3H) ppm. ESI-MS m/z calc. 232.9852, found 236.0 (M+1)⁺; Retention time: 1.5 minutes.

### Step 2:

TBSOTf (5 mL, 22.698 mmol) and 2,6 lutidine (1.08 mL, 9.332 mmol) were successively added to a solution of 2-(4-bromopyridin-2-yl)-1-fluoropropan-2-ol (700 mg, 2.99 mmol) in DMF (5 mL). The reaction mixture was stirred at ambient temperature for 2 days. Water was added and the mixture was extracted with EtOAc (2×50 mL). The combined organic extracts were washed with water (3×25 mL), brine (1×25 mL), dried (Na₂SO₄), filtered and concentrated in vacuo. Purification by flash chromatography (SiO₂, 5% EtOAc in hexanes) gave 4-bromo-2-(2-((tert-butyldimethylsilyl)oxy)-1-fluoropropan-2-yl)pyridine (350 mg, 34%). ¹H NMR (400 MHz, DMSO-d₆) δ 8.45 (d, J=5.2 Hz, 1H), 7.83 (d, J=1.4 Hz, 1H), 7.62 (dd, J=1.68 Hz, 1H), 4.66-4.48 (m, 2H), 1.52 (d, J=1.84 Hz, 3H), 0.93 (s, 9H), 0.12 (d, J=10.36 Hz, 6H) ppm. ESI-MS m/z calc. 347.0716, found 350.0 (M+1)⁺; Retention time: 2.15 minutes.

### Step 3:

Benzyl carbamate (249 mg, 1.647 mmol) and Cs₂CO₃ (640 mg, 1.9643 mmol) were successively added to a solution of 4-bromo-2-(2-((tert-butyldimethylsilyl)oxy)-1-fluoropropan-2-yl)pyridine (350 mg, 1.005 mmol) in 1,4-dioxane (10 mL). The mixture was degassed with argon for 2 min before addition of Pd₂(dba)₃ (65 mg, 0.07 mmol) and XPhos (72 mg, 0.151 mmol). The reaction mixture was heated at 90° C. for 1.5 h. The reaction mixture was filtered and washed with water (10 mL), brine (40 mL), dried (Na₂SO₄) and concentrated in vacuo. Purification by flash chromatography (SiO₂, 20% EtOAc in hexanes) gave benzyl (2-(2-((tert-butyldimethylsilyl)oxy)-1-fluoropropan-2-yl)pyridin-4-yl)carbamate (370 mg, 88%). ¹H NMR (400 MHz, DMSO-d₆) δ 10.32 (s, 1H), 8.33 (d, J=5.44 Hz, 1H), 7.82 (s, 1H), 7.45-7.33 (m, 6H), 5.17 (s, 2H), 4.62 (d, J=5.48 Hz, 1H), 4.50 (d, J=6.32 Hz, 1H), 1.48 (s, 3H), 0.93 (s, 9H), 0.10 (s, 6H) ppm. ESI-MS m/z calc. 418.2088, found 419.0 (M+1)⁺; Retention time: 2.15 minutes.

### Step 4:

Pd/C (80 mg, 10% w/w, 0.075 mmol) was added to a solution of benzyl (2-(2-((tert-butyldimethylsilyl)oxy)-1-fluoropropan-2-yl)pyridin-4-yl)carbamate (370 mg, 0.884 mmol) in EtOH (10 mL) under a nitrogen atmosphere. The reaction mixture was stirred under a hydrogen atmosphere at ambient temperature for 3 h before being filtered through a pad of Celite and concentrated in vacuo to give 2-(2-((tert-butyldimethylsilyl)oxy)-1-fluoropropan-2-yl)pyridin-4-amine (205 mg, 81%) as a brown solid. ¹H NMR (400 MHz, DMSO-d₆) δ 7.91 (d, J=6.6 Hz, 1H), 6.75 (d, J=2.0 Hz, 1H), 6.35 (dd, J=2.1 Hz, 5.5 Hz, 1H), 6.02 (s, 2H), 4.55 (d, J=1.7 Hz, 1H), 4.43 (d, J=1.1 Hz, 1H), 1.45 (d, J=1.6 Hz, 3H), 0.92 (s, 9H), 0.07 (s, 6H) ppm. ESI-MS m/z calc. 284.172, found 285.0 (M+1)⁺; Retention time: 3.36 minutes.

The following intermediate was made using a method similar to that described in Intermediate DE, except that 4,6-dibromopyrimidine was used as starting material for Step 1:

| Intermediate | | | |
|---|---|---|---|
| No. | Compound Name | LC/MS | NMR (shifts in ppm) |
| DF | 6-(2-((tert- | ESI-MS m/z calc. | ¹H NMR (400 MHz, DMSO-d₆) δ |
| | butyldimethylsilyl)oxy)- | 270.1564, found | 8.28 (s, 1H), 6.91 (br s, 2H), 6.64 |
| | 1-fluoropropan-2- | 271.0 (M + 1)⁺; | (s, 1H), 4.61-4.39 (m, 2H), 1.41 |
| | y1)pyrimidin-4-amine | Retention time: | (s, 3H), 0.93 (s, 9H), 0.12 (s, 6H) |
| | | 2.01 minutes | ppm. |

### Intermediate DG

### 2-(1-((tert-butyldimethylsilyl)oxy)-2-fluoroethyl)pyridin-4-amine

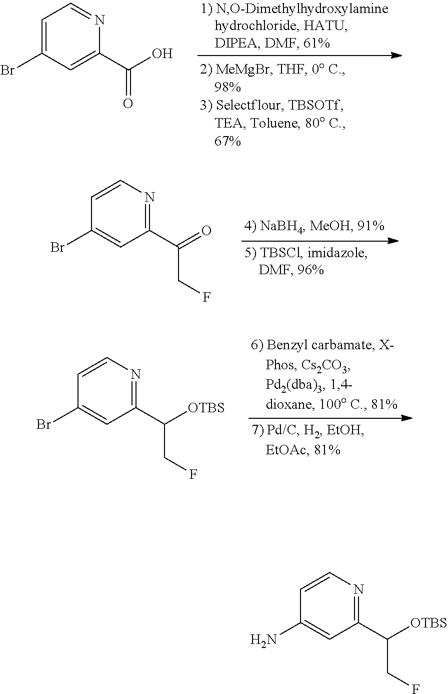 [SMILES: O=C(O)c1cc(Br)ccn1 ; O=C(CF)c1cc(Br)ccn1 ; CC(C)(C)[Si](C)(C)OC(CF)c1cc(Br)ccn1 ; CC(C)(C)[Si](C)(C)OC(CF)c1cc(N)ccn1]

### Step 1:

HATU (12.5 g, 32.875 mmol) was added to a solution of 4-bromopicolinic acid (5 g, 24.752 mmol) and DIPEA (22 mL, 126.3 mmol) in DMF (50 mL). The reaction mixture was stirred at ambient temperature for 30 min. N,O-dimethylhydroxylamine hydrochloride (3 g, 30.755 mmol) was then added and the mixture was stirred at ambient temperature for 24 h. The mixture was partitioned between water (200 mL) and EtOAc (200 mL). The organic phase was separated and concentrated in vacuo. Purification by flash chromatography (SiO₂, 10% EtOAc in hexanes) gave 4-bromo-N-methoxy-N-methylpicolinamide (4 g, 61%). ¹H NMR (400 MHz, DMSO-d₆) δ 8.49 (d, J=5.28 Hz, 1H), 7.86 (s, 1H), 7.79 (d, J=3.8 Hz, 1H), 3.64 (s, 3H), 3.27 (s, 3H) ppm. ESI-MS m/z calc. 243.9847, found 244.6 (M+1)⁺; Retention time: 2.43 minutes.

### Step 2:

MeMgBr (18.5 mL, 3 M solution in Et₂O, 55.5 mmol) was added dropwise to a solution of 4-bromo-N-methoxy-N-methylpicolinamide (9 g, 36.724 mmol) in THF (100 mL) at 0° C. The reaction mixture was stirred at 0° C. for 2 h. A cold aqueous NH₄Cl solution (250 mL) was added and the reaction mixture was extracted with EtOAc (500 mL). The organic extract was separated and concentrated in vacuo. Purification by flash chromatography (SiO₂, 20% EtOAc in hexanes) gave 1-(4-bromopyridin-2-yl)ethan-1-one (7.2 g, 98%). ¹H NMR (400 MHz, DMSO-d₆) δ 8.62 (d, J=5.2 Hz, 1H), 8.06 (s, 1H), 7.96-7.94 (m, 1H), 2.63 (s, 3H) ppm.

### Step 3:

TBSOTf (598 mg, 0.52 mL, 2.262 mmol) was added dropwise to a solution of 1-(4-bromopyridin-2-yl)ethan-1-one (300 mg, 1.5 mmol) and Et₃N (0.45 mL, 3.22 mmol) in toluene (5 mL). The reaction mixture was heated to 80° C. for 2 h. The upper toluene phase was separated and concentrated in vacuo. The residue was dissolved in MeCN (5 mL) and Selectfluor (535 mg, 1.510 mmol) was added. The reaction mixture was stirred at ambient temperature for 1 h and concentrated in vacuo. The residue was partitioned between water and EtOAc (2×20 mL). The organic extract was dried (Na₂SO₄) and concentrated in vacuo. Purification by flash chromatography (SiO₂, 0 to 10% EtOAc in hexanes) gave 1-(4-bromopyridin-2-yl)-2-fluoroethan-1-one (220 mg, 67%). ¹H NMR (400 MHz, DMSO-d₆) δ 8.59 (d, J=5.2 Hz, 1H), 8.12 (s, 1H), 8.03-7.99 (m, 1H), 5.91 (d, J=46.8 Hz, 2H) ppm.

### Step 4:

NaBH₄ (1.2 g, 1.27 mL, 31.719 mmol) was added to a solution of 1-(4-bromopyridin-2-yl)-2-fluoroethan-1-one (3.5 g, 16.05 mmol) in MeOH (60 mL) at 0° C. The reaction mixture was stirred at ambient temperature for 3 h. The mixture was concentrated in vacuo and partitioned between water (100 mL) and EtOAc (200 mL). The organic extract was separated, dried (Na₂SO₄) and concentrated in vacuo. Purification by flash chromatography (SiO₂, 0 to 50% EtOAc in hexanes) gave 1-(4-bromopyridin-2-yl)-2-fluoroethan-1-ol (3.2 g, 91%). ¹H NMR (400 MHz, DMSO-d₆) δ 8.42 (d, J=5.2 Hz, 1H), 7.75 (s, 1H), 7.62-7.58 (m, 1H), 6.0 (d, J=5.2 Hz, 1H), 4.89-4.81 (m, 1H), 4.74-4.51 (m, 2H) ppm.

### Step 5:

TBSCl (4.1 g, 27.202 mmol) was added portionwise to a solution of 1-(4-bromopyridin-2-yl)-2-fluoroethan-1-ol (3.5 g, 15.9 mmol) and imidazole (4.0 g, 58.757 mmol) in DMF (20 mL). The reaction mixture was stirred at ambient temperature for 2 h, then partitioned between ice cooled water (80 mL) and EtOAc (250 mL). The organic extract was separated, dried (Na₂SO₄) and concentrated in vacuo. Purification by flash chromatography (SiO₂, 0 to 10% EtOAc in hexanes) gave 4-bromo-2-(1-((tert-butyldimethylsilyl)oxy)-2-fluoroethyl)pyridine (5.1 g, 96%) as pale yellow oil. ¹H NMR (400 MHz, DMSO-d₆) δ 8.44 (d, J=5.6 Hz, 1H), 7.69 (s, 1H), 7.65-7.61 (m, 1H), 5.08-5.01 (m, 1H), 4.71-4.43 (m, 2H), 0.88 (s, 9H), 0.10 (s, 3H), 0.02 (s, 3H) ppm.

### Step 6:

Cs₂CO₃ (8.5 g, 26.088 mmol) and benzyl carbamate (3.3 g, 21.831 mmol) were successively added to a solution of 4-bromo-2-(1-((tert-butyldimethylsilyl)oxy)-2-fluoroethyl)pyridine (4.8 g, 14.358 mmol) in 1,4-dioxane (120 mL). The mixture was degassed with argon. Xantphos (335 mg, 0.579 mmol) and Pd₂(dba)₃ (165 mg, 0.287 mmol) were added and the reaction mixture was heated at 100° C. for 3 h. The mixture was filtered through a pad of Celite. The filtrates were partitioned between water and EtOAc. The organic extract was separated and concentrated in vacuo. Purification by flash chromatography (SiO₂, 0 to 50% EtOAc in hexanes) gave benzyl (2-(1-((tert-butyldimethylsilyl)oxy)-2-fluoroethyl)pyridin-4-yl)carbamate (4.7 g, 81%) as a colourless oil. ¹H NMR (400 MHz, DMSO-d₆) δ 10.33 (s, 1H), 8.32 (d, J=5.6 Hz, 1H), 7.67 (s, 1H), 7.68-7.33 (m, 6H), 5.17 (s, 2H), 4.99-4.91 (m, 1H), 4.69-4.33 (m, 2H), 0.90 (s, 9H), 0.07 (s, 3H), 0.04 (s, 3H) ppm.

### Step 7:

Pd/C (500 mg, 10% w/w, 0.075 mmol) was added to a solution of benzyl (2-(1-((tert-butyldimethylsilyl)oxy)-2-fluoroethyl)pyridin-4-yl)carbamate (4.6 g, 11.371 mmol) in EtOH (30 mL) and EtOAc (30 mL) under a nitrogen atmosphere. The reaction mixture was stirred under a hydrogen atmosphere (60 psi) in the parr shaker for 3 h, filtered through a pad of Celite and concentrated in vacuo. Purification by flash chromatography (SiO₂, 0 to 40% EtOAc in hexanes) gave 2-(1-((tert-butyldimethylsilyl)oxy)-2-fluoroethyl)pyridin-4-amine (2.5 g, 81%) as white solid. ¹H NMR (400 MHz, DMSO-d₆) δ 7.89 (d, J=5.6 Hz, 1H), 6.64 (s, 1H), 6.36-6.32 (m, 1H), 6.06 (s, 2H), 4.77-4.74 (m, 1H), 4.63-4.24 (m, 2H), 0.89 (s, 9H), 0.06 (s, 3H), 0.03 (s, 3H) ppm. ESI-MS m/z calc. 270.1564, found 271.0 (M+1)⁺; Retention time: 2.01 minutes.

### Intermediate DH

### 5-fluoro-2-(2,2,3,3,9,9,10,10-octamethyl-4,8-dioxa-3,9-disilaundecan-5-yl)pyridin-4-amine

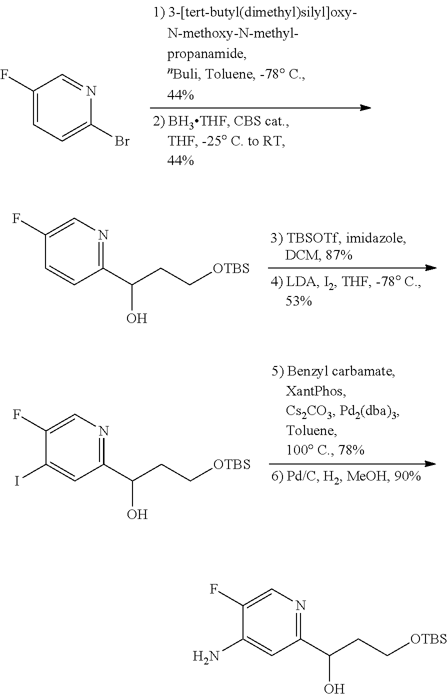 [SMILES: Fc1ccc(Br)nc1 ; CC(C)(C)[Si](C)(C)OCCC(O)c1ccc(F)cn1 ; CC(C)(C)[Si](C)(C)OCCC(O)c1cc(I)c(F)cn1 ; CC(C)(C)[Si](C)(C)OCCC(O)c1cc(N)c(F)cn1]

### Step 1:

ⁿBuLi (7.5 mL, 1.81 M in hexane, 13.575 mmol) was added dropwise to a solution of 2-bromo-5-fluoro-pyridine (2 g, 11.365 mmol) in toluene (60 mL) at −78° C. The reaction mixture was stirred for 45 min at −78° C. A solution of 3-[tert-butyl(dimethyl)silyl]oxy-N-methoxy-N-methyl-propanamide (3.5 g, 14.147 mmol) in toluene (5 mL) was added dropwise and the mixture was stirred for 45 min at −78° C. An aqueous NH₄Cl solution (100 mL) was added slowly and the mixture was extracted with EtOAc (200 mL). The organic extracts were dried (Na₂SO₄) and concentrated in vacuo. Purification by flash chromatography (SiO₂, 5% EtOAc in hexanes) gave 3-((tert-butyldimethylsilyl)oxy)-1-(5-fluoropyridin-2-yl)propan-1-one (1.5 g, 44%). ¹H NMR (400 MHz, DMSO-d₆) δ 8.73 (s, 1H), 8.04 (d, J=4.52 Hz, 1H), 7.92 (t, J=8.72 Hz, 1H), 3.99 (t, J=6.0 Hz, 2H), 3.28 (t, J=5.92 Hz, 2H), 0.79 (s, 9H), -0.01 (s, 6H) ppm. ESI-MS m/z calc. 283.1404, found 284.2 (M+1)⁺; Retention time: 2.25 minutes.

### Step 2:

1 M BH₃ (0.78 mL, solution in THF, 0.78 mmol) was added to a 1 M solution of (3aR)-1-methyl-3,3-diphenyl-3a,4,5,6-tetrahydropyrrolo[1,2-c][1,3,2]oxazaborole (0.07 mL, solution in toluene, 0.07 mmol) in THF (4 mL). The reaction mixture was cooled to −25° C. and a solution of 3-((tert-butyldimethylsilyl)oxy)-1-(5-fluoropyridin-2-yl)propan-1-one (200 mg, 0.706 mmol) in THF (2 mL) was added. The reaction mixture was warmed to ambient temperature and stirred for 3 h. The reaction mixture was concentrated in vacuo. Purification by flash chromatography (SiO₂, 5 to 10% EtOAc in hexanes) gave 3-((tert-butyldimethylsilyl)oxy)-1-(5-fluoropyridin-2-yl)propan-1-ol (90 mg, 44%). ¹H NMR (400 MHz, DMSO-d₆) δ 8.46 (s, 1H), 7.71-7.66 (m, 1H), 7.55-7.53 (m, 1H), 5.39 (d, J=5.12 Hz, 1H), 4.69 (t, J=4.36, 1H), 3.79-3.73 (m, 1H), 3.67-3.62 (m, 1H), 1.95-1.89 (m, 1H), 1.77-1.70 (m, 1H), 0.85 (s, 9H), 0.02 (s, 6H) ppm. ESI-MS m/z calc. 285.156, found 285.9 (M+1)⁺; Retention time: 1.86 minutes.

### Step 3:

TBSCl (872.5 mg, 5.7888 mmol) and imidazole (790.3 mg, 11.609 mmol) were successively added to a solution of 3-((tert-butyldimethylsilyl)oxy)-1-(5-fluoropyridin-2-yl)propan-1-ol (1.1 g, 3.8538 mmol) in DCM (27 mL). The reaction mixture was stirred at ambient temperature overnight. Water (30 mL) was added and the mixture was extracted with DCM (3×25 mL). The combined organic extracts were washed with water (30 mL), brine (30 mL), dried (Na₂SO₄) and concentrated in vacuo. Purification by flash chromatography (SiO₂, 15% EtOAc in hexanes) gave 5-fluoro-2-(2,2,3,3,9,9,10,10-octamethyl-4,8-dioxa-3,9-disilaundecan-5-yl)pyridine (1.35 g, 87%) as yellow oil. ¹H NMR (250 MHz, Chloroform-d) δ 8.35 (d, J=2.8 Hz, 1H), 7.42 (ddt, J=11.1, 8.4, 3.8 Hz, 2H), 4.91 (t, J=6.2 Hz, 1H), 3.82-3.55 (m, 2H), 1.91 (p, J=6.6 Hz, 2H), 0.88 (d, J=4.3 Hz, 18H), 0.10-0 (m, 12H) ppm. ESI-MS m/z calc. 399.2425, found 400.5 (M+1)⁺; Retention time: 4.85 minutes.

### Step 4:

ⁿBuLi (0.35 mL, 2.5 M in hexanes, 0.8750 mmol) was added to a solution of diisopropylamine (128 μL, 0.913 mmol) in THF (4 mL) at −78° C. The reaction mixture was stirred at 0° C. for 30 min and cooled to −78° C. A solution of 5-fluoro-2-(2,2,3,3,9,9,10,10-octamethyl-4,8-dioxa-3,9-disilaundecan-5-yl)pyridine (300 mg, 0.75 mmol) in THF (1.5 mL) was added dropwise and the reaction mixture was stirred at −78° C. for 30 min, and then at −50° C. for 1 h. The reaction mixture was cooled to -78° C., and a solution of I₂ (224.8 mg, 0.8857 mmol) in THF (1.5 mL) was added. The reaction mixture was stirred at −78° C. for 30 min, then at ambient temperature overnight. The mixture was poured into a saturated NaHCO₃ solution (15 mL) and extracted with Et₂O (3×15 mL). The combined organic extracts were washed with water (20 mL), brine (20 mL), dried (Na₂SO₄), filtered and concentrated in vacuo. Purification by flash chromatography (24 g SiO₂, 0 to 100% DCM in hexanes) gave 5-fluoro-4-iodo-2-(2,2,3,3,9,9,10,10-octamethyl-4,8-dioxa-3,9-disilaundecan-5-yl)pyridine (216.5 mg, 53%) as light yellow oil. ¹H NMR (400 MHz, DMSO-d₆) δ 8.21 (s, 1H), 7.88 (d, J=5.0 Hz, 1H), 4.87 (dd, J=7.6, 4.7 Hz, 1H), 3.83-3.53 (m, 2H), 1.90 (p, J=7.1, 7.1, 6.9, 6.9 Hz, 2H), 0.89 (d, J=8.6 Hz, 18H), 0.14-−0.15 (m, 12H) ppm. ESI-MS m/z calc. 525.1392, found 526.7 (M+1)⁺; Retention time: 5.17 minutes.

### Step 5:

Benzyl carbamate (31.2 mg, 0.206 mmol), Cs₂CO₃ (80.7 mg, 0.247 mmol), Pd₂(dba)₃ (7.1 mg, 0.008 mmol) and XantPhos (8.8 mg, 0.015 mmol) were successively added to a solution of 5-fluoro-4-iodo-2-(2,2,3,3,9,9,10,10-octamethyl-4,8-dioxa-3,9-disilaundecan-5-yl)pyridine (100.1 mg, 0.19 mmol) in toluene (1.5 mL). The reaction mixture was degassed with nitrogen and heated at 100° C. under microwave irradiations for 12 h. The reaction mixture was filtered through Celite and concentrated in vacuo. Purification by flash chromatography (12 g SiO₂, 0 to 25% EtOAc in hexanes) gave benzyl (5-fluoro-2-(2,2,3,3,9,9,10,10-octamethyl-4,8-dioxa-3,9-disilaundecan-5-yl)pyridin-4-yl)carbamate (81.8 mg, 78%) as light yellow oil. ¹H NMR (250 MHz, Chloroform-d) δ 8.33-8.22 (m, 2H), 7.46-7.35 (m, 5H), 7.09 (s, 1H), 5.25 (s, 2H), 4.85 (dt, J=5.1, 2.8 Hz, 1H), 3.82-3.59 (m, 2H), 1.91 (m, 2H), 0.92 (s, 9H), 0.88 (s, 9H), 0.08-−0.10 (m, 12H) ppm. ESI-MS m/z calc. 548.2902, found 549.7 (M+1)⁺; Retention time: 4.33 minutes.

### Step 6:

Pd/C (1.85 g, 10% w/w, 1.7384 mmol) was added to a solution of benzyl (5-fluoro-2-(2,2,3,3,9,9,10,10-octamethyl-4,8-dioxa-3,9-disilaundecan-5-yl)pyridin-4-yl)carbamate (6.42 g, 11.697 mmol) in MeOH (110 mL) under a nitrogen atmosphere. The reaction mixture was stirred under a hydrogen atmosphere for 1.5 h, filtered through a pad of Celite and concentrated in vacuo. Purification by flash chromatography (120 g SiO₂, 0 to 25% EtOAc in hexanes) gave 5-fluoro-2-(2,2,3,3,9,9,10,10-octamethyl-4,8-dioxa-3,9-disilaundecan-5-yl)pyridin-4-amine (4.72 g, 90%) as light yellow solid. ¹H NMR (400 MHz, DMSO-d₆) δ 7.95 (d, J=3.4 Hz, 1H), 6.76 (d, J=7.7 Hz, 1H), 6.13 (s, 2H), 4.66 (dd, J=8.2, 4.0 Hz, 1H), 3.69 (q, J=8.1, 8.1, 7.8 Hz, 1H), 3.62-3.54 (m, 1H), 1.82-1.65 (m, 2H), 0.86 (s, 18H), −0.05 (m, 12H) ppm. ESI-MS m/z calc. 414.2534, found 415.6 (M+1)⁺; Retention time: 3.12 minutes.

### Intermediate DI

### 2-(2-((tert-butyldiphenylsilyl)oxy)ethyl)pyrimidin-5-amine

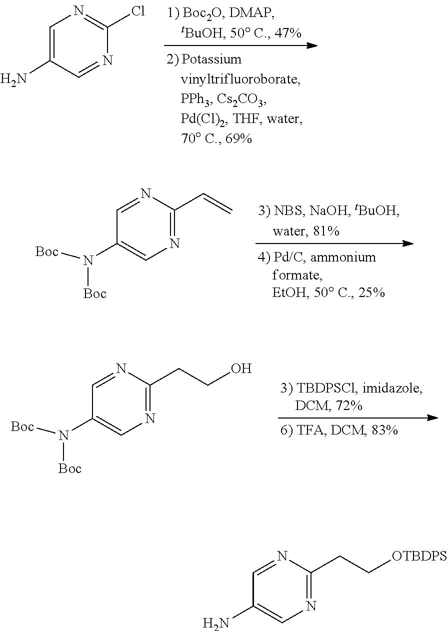

### Step 1:

A mixture of 2-chloropyrimidin-5-amine (10 g, 77.191 mmol), Boc₂0 (51 g, 53.684 mL, 233.68 mmol) and DMAP (555 mg, 4.543 mmol) in ^{t}BuOH (200 mL) was heated at 50° C. for 8 h. After cooling to ambient temperature, hexanes (500 mL) were added and the mixture was filtered. The filtered cake was washed with hexanes and dried to give tert-butyl (tert-butoxycarbonyl)(2-chloropyrimidin-5-yl)carbamate (17.1 g, 47%) as a light brown solid. ¹H NMR (250 MHz, Chloroform-d) δ 8.47 (s, 2H), 1.46 (s, 18H) ppm. ESI-MS m/z calc. 329.1142, found 330.0 (M+1)⁺; Retention time: 3.21 minutes.

### Step 2:

Potassium vinyltrifluoroborate (593 mg, 4.42 mmol) and triphenylphosphine (78 mg, 0.068 mL, 0.297 mmol) were successively added to a suspension of tert-butyl (tert-butoxycarbonyl)(2-chloropyrimidin-5-yl)carbamate (990 mg, 2.852 mmol) and Cs₂CO₃ (2.4 g, 7.366 mmol) in a mixture of water (2 mL) and THF (20 mL). Nitrogen was bubbled through the mixture and then PdCl₂ (26 mg, 0.146 mmol) was added. The reaction mixture was heated at 70° C. for 36 h. The mixture was diluted with water and extracted with EtOAc (2×70 mL). The combined organic extracts were dried (Na₂SO₄), filtered and concentrated in vacuo. Purification by flash chromatography (80 g SiO₂, 0 to 30% EtOAc in Hexanes) gave tert-butyl (tert-butoxycarbonyl)(2-vinylpyrimidin-5-yl)carbamate (633 mg, 69%) as a white solid. ¹H NMR (300 MHz, Chloroform-d) δ 8.50 (s, 2H), 6.95-6.84 (m, 1H), 6.67-6.58 (m, 1H), 5.77 (dd, J=10.5 Hz, 1.7 Hz, 1H), 1.43 (s, 18H) ppm. ESI-MS m/z calc. 321.1689, found 322.2 (M+1)⁺; Retention time: 2.03 minutes.

### Step 3:

NBS (333 mg, 1.871 mmol) was added portionwise to a solution of tert-butyl (tert-butoxycarbonyl)(2-vinylpyrimidin-5-yl)carbamate (500 mg, 1.501 mmol) in a mixture of ^{t}BuOH (3 mL) and water (9 mL). The reaction mixture was stirred at ambient temperature for 1 h and 45 min. 2 M NaOH (2.3 mL, 4.6 mmol) was added and the reaction mixture was stirred for 45 min. The mixture was extracted with EtOAc (2×30 mL). The combined organic extracts were dried (Na₂SO₄), filtered and concentrated in vacuo to give tert-butyl N-tert-butoxycarbonyl-N-[2-(oxiran-2-yl)pyrimidin-5-yl]carbamate (554 mg, 81%). ¹H NMR (300 MHz, Chloroform-d) δ 8.53 (s, 2H), 4.16-4.10 (m, 1H), 3.27-3.17 (m, 2H), 1.43 (s, 18H) ppm. ESI-MS m/z calc. 337.1638, found 338.2 (M+1)⁺; Retention time: 1.89 minutes.

### Step 4:

Pd/C (258 mg, 0.1212 mmol, 50% wet) and ammonium formate (355 mg, 5.629 mmol) were successively added to a solution of tert-butyl N-tert-butoxycarbonyl-N-[2-(oxiran-2-yl)pyrimidin-5-yl]carbamate (554 mg, 1.215 mmol) in EtOH (10 mL). The reaction mixture was heated at 50° C. for 2 h. The mixture was filtered and concentrated in vacuo. Purification by flash chromatography (80 g SiO₂, 0 to 50% EtOAc in heptane) gave tert-butyl (tert-butoxycarbonyl)(2-(2-hydroxyethyl)pyrimidin-5-yl)carbamate (103 mg, 25%) as a white solid. ¹H NMR (300 MHz, Chloroform-d) δ 8.49 (s, 2H), 4.08 (t, J=5.2 Hz, 2H), 3.25 (t, J=5.2 Hz, 2H), 1.45 (s, 18H) ppm; OH alcohol not observed. ESI-MS m/z calc. 339.1794, found 340.2 (M+1)⁺; Retention time: 1.75 minutes.

### Step 5:

TBDPSCl (1.9 mL, 7.307 mmol) was added to a solution of tert-butyl (tert-butoxycarbonyl)(2-(2-hydroxyethyl)pyrimidin-5-yl)carbamate (1.9 g, 5.598 mmol) and imidazole (438 mg, 6.434 mmol) in DCM (95 mL). The reaction mixture was stirred at ambient temperature for 18 h. Additional imidazole (191 mg, 2.8 mmol) and TBDPSCl (0.5 mL, 1.923 mmol) were added and the reaction was stirred for a further 2 h. Additional TBDPSCl (0.5 mL, 1.923 mmol) was added and the reaction was stirred for another 18 h. The mixture was washed with water (150 mL) and brine (100 mL). The organic phase was collected, dried (Na₂SO₄), filtered and concentrated in vacuo. Purification by flash chromatography (80 g SiO₂, 0 to 25% EtOAc in hexanes) gave tert-butyl (tert-butoxycarbonyl)(2-(2-((tert-butyldiphenylsilyl)oxy)ethyl)pyrimidin-5-yl)carbamate (2.317 g, 72%) as a white solid. ¹H NMR (300 MHz, Chloroform-d) δ 8.47 (s, 2H), 7.67-7.60 (m, 4H), 7.43-7.38 (m, 6H), 4.19 (t, J=6.9 Hz, 2H), 3.25 (t, J=6.5 Hz, 2H), 1.40 (s, 18H), 0.97 (s, 9H) ppm. ESI-MS m/z calc. 577.2972; Retention time 2.77 minutes.

### Step 6:

Step 6 was carried out according to General Method I to give 2-(2-((tert-butyldiphenylsilyl)oxy)ethyl)pyrimidin-5-amine (1.25 g, 83%) as a clear oil. ¹H NMR (300 MHz, Chloroform-d) δ 8.12 (s, 2H), 7.67-7.55 (m, 4H), 7.45-7.29 (m, 6H), 4.11 (t, J=6.9 Hz, 2H), 3.56 (br s, 2H), 3.14 (t, J=6.8 Hz, 2H), 0.98 (s, 9H) ppm. ESI-MS m/z calc. 377.1923, found 378.2 (M+1)⁺; Retention time: 3.32 minutes.

### Intermediate DJ

(R)-1-(2-((tert-butyldimethylsilyl)oxy)-3-methoxypropyl)-3-methyl-1H-pyrazol-4-amine

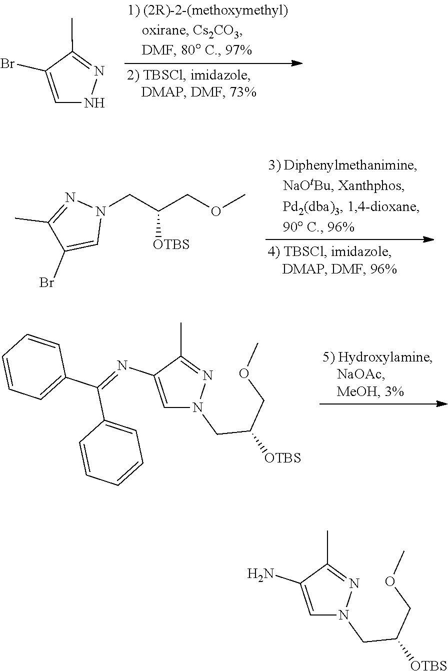

### Step 1:

Cs₂CO₃ (12 g, 36.83 mmol) was added to a solution of 4-bromo-3-methyl-1H-pyrazole (5 g, 31.05 mmol) in DMF (50 mL). The mixture was stirred at ambient temperature for 15 min. (2R)-2-(methoxymethyl)oxirane (3 mL, 33.437 mmol) was added and the reaction mixture was heated at 80° C. for 6 h. Ice cold water was added and the mixture was extracted with EtOAc (250 mL). The organic extract was dried (Na₂SO₄) and concentrated in vacuo. Purification by flash chromatography (SiO₂, 20 to 40% EtOAc in hexane) gave (R)-1-(4-bromo-3-methyl-1H-pyrazol-1-yl)-3-methoxypropan-2-ol and (R)-1-(4-bromo-5-methyl-1H-pyrazol-1-yl)-3-methoxypropan-2-ol (7.5 g, 97%). ESI-MS m/z calc. 248.016, found 250.0 (M+1)⁺; Retention time: 2.47 minutes.

### Step 2:

TBSCl (13.5 g, 89.569 mmol) and DMAP (85 mg, 0.696 mmol) were successively added to a solution containing a mixture of (R)-1-(4-bromo-3-methyl-1H-pyrazol-1-yl)-3-methoxypropan-2-ol and (R)-1-(4-bromo-5-methyl-1H-pyrazol-1-yl)-3-methoxypropan-2-ol (7.5 g, 30.108 mmol) and imidazole (10.5 g, 154.24 mmol) in DMF (50 mL). The reaction mixture was stirred at ambient temperature for 2 h. Ice cold water was added and the mixture was extracted with EtOAc (250 mL). The organic extract was dried (Na₂SO₄) and concentrated in vacuo. Purification by flash chromatography (SiO₂, 0 to 10% EtOAc in hexanes) gave (R)-4-bromo-1-(2-((tert-butyldimethylsilyl)oxy)-3-methoxypropyl)-3-methyl-1H-pyrazole and (R)-4-bromo-1-(2-((tert-butyldimethylsilyl)oxy)-3-methoxypropyl)-5-methyl-1H-pyrazole (8 g, 73%) as a light yellow oil and as a mixture of regioisomers. ESI-MS m/z calc. 362.1025, found 363.0 (M+1)⁺; Retention time: 2.35 minutes.

### Step 3:

NaO^{t}Bu (2.2 g, 22.892 mmol) and diphenylmethanimine (1.5 g, 1.388 mL, 8.276 mmol) were successively added to a solution of (R)-4-bromo-1-(2-((tert-butyldimethylsilyl)oxy)-3-methoxypropyl)-3-methyl-1H-pyrazole and (R)-4-bromo-1-(2-((tert-butyldimethylsilyl)oxy)-3-methoxypropyl)-5-methyl-1H-pyrazole (2.7 g, 7.43 mmol) in 1,4-dioxane (30 mL). The reaction mixture was degassed for 5 min before addition of Xantphos (430 mg, 0.7432 mmol) and tris(dibenzylideneacetone)dipalladium(0) (340 mg, 0.3713 mmol). The reaction mixture was heated at 90° C. for 4 h. The mixture was partitioned between water and EtOAc (250 mL). The organic phase was separated, dried (Na₂SO₄) and concentrated in vacuo. Purification by flash chromatography (SiO₂, 20 to 40% EtOAc in hexanes) gave a mixture of (R)-1-(4-((diphenylmethylene)amino)-3-methyl-1H-pyrazol-1-yl)-3-methoxypropan-2-ol and (R)-1-(4-((diphenylmethylene)amino)-5-methyl-1H-pyrazol-1-yl)-3-methoxypropan-2-ol (2.5 g, 96%). ESI-MS m/z calc. 349.179, found 350.0 (M+1)⁺; Retention time: 1.88 minutes.

### Step 4:

TBSCl (8.2 g, 54.405 mmol) and DMAP (55 mg, 0.4502 mmol) were added to a solution of a mixture of (R)-1-(4-((diphenylmethylene)amino)-3-methyl-1H-pyrazol-1-yl)-3-methoxypropan-2-ol and (R)-1-(4-((diphenylmethylene)amino)-5-methyl-1H-pyrazol-1-yl)-3-methoxypropan-2-ol (6 g, 13.496 mmol) and imidazole (6 g, 88.135 mmol) in DMF (60 mL) under an argon atmosphere. The reaction mixture was stirred at ambient temperature for 2 h. Ice cold water was added and the mixture was extracted with EtOAc (250 mL). The organic extract was dried (Na₂SO₄) and concentrated in vacuo. Purification by flash chromatography (SiO₂, 0 to 10% EtOAc in hexanes) gave a mixture of (R)—N-(1-(2-((tert-butyldimethylsilyl)oxy)-3-methoxypropyl)-3-methyl-1H-pyrazol-4-yl)-1,1-diphenylmethanimine and (R)—N-(1-(2-((tert-butyldimethylsilyl)oxy)-3-methoxypropyl)-5-methyl-1H-pyrazol-4-yl)-1,1-diphenylmethanimine (6 g, 96%) as a yellow oil. ESI-MS m/z calc. 463.2655, found 464.0 (M+1)⁺; Retention time: 2.25 minutes.

### Step 5:

Sodium acetate (4 g, 48.76 mmol) and hydroxylamine (Hydrochloride salt) (2.7 g, 38.85 mmol) were successively added to a solution of (R)—N-(1-(2-((tert-butyldimethylsilyl)oxy)-3-methoxypropyl)-3-methyl-1H-pyrazol-4-yl)-1,1-diphenylmethanimine and (R)—N-(1-(2-((tert-butyldimethylsilyl)oxy)-3-methoxypropyl)-5-methyl-1H-pyrazol-4-yl)-1,1-diphenylmethanimine (6 g, 12.94 mmol) in MeOH (60 mL). The reaction mixture was stirred at ambient temperature for 1.5 h. The mixture was concentrated in vacuo. A mixture of a saturated aqueous NaHCO₃ solution (100 mL) and water (100 mL) was added and the mixture was extracted with EtOAc (500 mL). The organic extracts were collected, dried (Na₂SO₄) and concentrated in vacuo. Purification by flash chromatography (SiO₂, 50 to 100% EtOAc in hexanes) gave (R)-1-(2-((tert-butyldimethylsilyl)oxy)-3-methoxypropyl)-3-methyl-1H-pyrazol-4-amine (100 mg, 3%) and (R)-1-(2-((tert-butyldimethylsilyl)oxy)-3-methoxypropyl)-5-methyl-1H-pyrazol-4-amine (3.2 g, 82%). (R)-1-(2-((tert-butyldimethylsilyl)oxy)-3-methoxypropyl)-3-methyl-1H-pyrazol-4-amine: ¹H NMR (400 MHz, DMSO-d₆) δ 6.88 (s, 1H), 4.05-4.05 (m, 1H), 3.90 (dd, J=13.8, 4.36 Hz, 1H), 3.78-3.73 (m, 1H), 3.55 (s, 2H), 3.25 (s, 3H), 3.20 (t, J=5.8 Hz, 2H), 1.97 (s, 3H), 0.81 (s, 9H), −0.04 (s, 3H), −0.14 (s, 3H) ppm; ESI-MS m/z calc. 299.2029, found 300.0 (M+1)⁺; Retention time: 1.36 minutes. (R)-1-(2-((tert-butyldimethylsilyl)oxy)-3-methoxypropyl)-5-methyl-1H-pyrazol-4-amine: ESI-MS m/z calc. 299.2029, found 300.0 (M+1); Retention time: 2.01 minutes.

The following intermediates were made using a method similar to that described in Intermediate DJ, except that (2S)-2-(methoxymethyl)oxirane was used as starting material for Step 1:

| Intermediate | | | |
|---|---|---|---|
| No. | Compound Name | LC/MS | NMR (shifts in ppm) |
| DK | (S)-1-(2-((tert- | ESI-MS m/z calc. | ¹H NMR (400 MHz, DMSO-d₆) δ |
| | butyldimethylsilyl)oxy)- | 299.2029, found | 6.92 (s, 1H), 4.13-4.05 (m, 1H), |
| | 3-methoxypropyl)-3- | 300.3 (M + 1)⁺; | 3.94 (dd, J = 13.8, 4.6 Hz, 1H), |
| | methyl-1H-pyrazol-4- | Retention time: | 3.81 (dd, J = 13.8, 7.1 Hz, 1H), |
| | amine | 2.79 minutes | 3.57 (s, 2H), 3.29 (s, 3H), 3.26 |
| | | | (dd, J = 8.2, 5.1 Hz, 1H), 2.02 |
| | | | (s, 3H), 0.85 (s, 10H), 0.00 (s, |
| | | | 3H), −0.09 (s, 3H) ppm. |
| DL | (S)-1-(2-((tert- | ESI-MS m/z calc. | ¹H NMR (400 MHz, DMSO-d₆) δ |
| | butyldimethylsilyl)oxy)- | 299.2029, found | 6.96 (s, 1H), 4.17-4.13 (m, 1H), |
| | 3-methoxypropyl)-5- | 300.3 (M + 1)⁺; | 3.99 (dd, J = 14.1, 4.5 Hz, 1H), |
| | methyl-1H-pyrazol-4- | Retention time: | 3.89 (dd, J = 14.1, 7.8 Hz, 1H), |
| | amine | 2.84 minutes | 3.54 (s, 2H), 3.36-3.30 (m, 2H),, |
| | | | 3.33 (s, 3H), 2.13 (s, 3H), 0.86 |
| | | | (s, 9H), 0.00 (s, 3H), −0.15 |
| | | | (s, 3H) ppm. |

### Intermediate DM

### 6-(((tert-butyldimethylsilyl)oxy)methyl)pyrimidin-4-amine

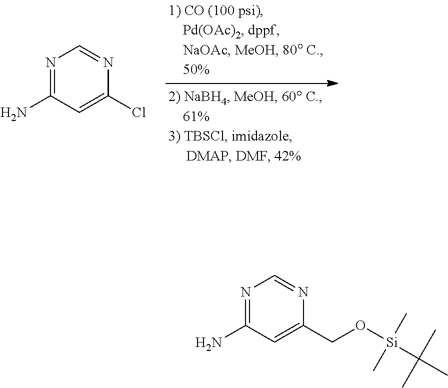 [SMILES: Nc1cc(Cl)ncn1 ; CC(C)(C)[Si](C)(C)OCc1cc(N)ncn1]

### Step 1:

NaOAc (17 g, 207.23 mmol) and dppf (4.3 g, 7.756 mmol) were successively added to a solution of 6-chloropyrimidin-4-amine (10 g, 77.191 mmol) in MeOH (450 mL). The reaction mixture was degassed for 10 min with argon. Pd(OAc)₂ (1 g, 4.45 mmol) was added and the reaction mixture was heated at 80° C. for 16 h under a carbon monoxide atmosphere (100 psi). The reaction mixture was filtered through a pad of Celite and washed with MeOH (500 mL). The filtrates were concentrated in vacuo. Purification by flash chromatography (SiO₂, 20% MeOH in EtOAc) gave methyl 6-aminopyrimidine-4-carboxylate (7 g, 50%). ¹H NMR (400 MHz, DMSO-d₆) δ 8.44 (s, 1H), 7.27 (s, 2H), 7.03 (s, 1H), 3.83 (s, 3H) ppm. ESI-MS m/z calc. 153.0538, found 154.1 (M+1); Retention time: 1.03 minutes.

### Step 2:

NaBH₄ (8.5 g, 8.994 mL, 224.67 mmol) was added portionwise to a solution of methyl 6-aminopyrimidine-4-carboxylate (7 g, 45.71 mmol) in MeOH (200 mL) at 0° C. The reaction mixture was heated to reflux for 2 h. The mixture was concentrated in vacuo. Purification by flash chromatography (SiO₂, 20% MeOH in EtOAc) gave (6-aminopyrimidin-4-yl)methanol (3.5 g, 61%). ¹H NMR (400 MHz, DMSO-d₆) δ 8.20 (s, 1H), 6.77 (s, 2H), 6.52 (s, 1H), 5.32 (t, J=5.4, 1H), 4.29 (d, J=5.68, 2H) ppm.

### Step 3:

TBSCl (8.4 g, 55.732 mmol) was added portionwise to a solution of (6-aminopyrimidin-4-yl)methanol (3.5 g, 27.971 mmol), Et₃N (11.708 mL, 84 mmol) and DMAP (342 mg, 2.79 mmol) in DMF (50 mL) at 0° C. The reaction mixture was stirred at ambient temperature for 2 h. Ice cold water was added and the mixture was extracted with EtOAc (250 mL). The organic extract was dried (Na₂SO₄) and concentrated in vacuo. Purification by flash chromatography (SiO₂, 20 to 30% EtOAc in hexanes) gave 6-(((tert-butyldimethylsilyl)oxy)methyl)pyrimidin-4-amine (2.83 g, 42%) as an off-white solid. ¹H NMR (400 MHz, DMSO-d₆) δ 8.21 (s, 1H), 6.83 (s, 2H), 6.50 (s, 1H), 4.48 (s, 2H), 0.92 (s, 9H), 0.09 (s, 6H) ppm. ESI-MS m/z calc. 239.1454, found 240.2 (M+1)⁺; Retention time: 1.94 minutes.

### Intermediate DN

### 3-((4-aminopyridin-2-yl)oxy)-1-methylpyrrolidin-2-one

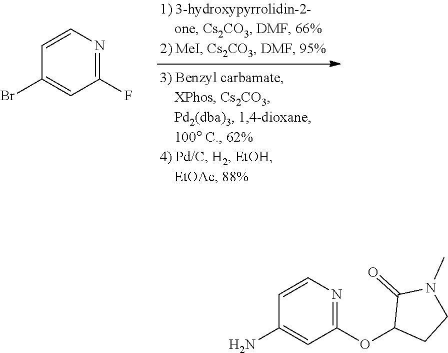

### Step 1:

Cs₂CO₃ (23.1 g, 70.898 mmol) was added to a solution of 3-hydroxypyrrolidin-2-one (3.3 g, 32.64 mmol) in DMF (100 mL) at 0° C. The reaction mixture was stirred at ambient temperature for 1 h. A solution of 4-bromo-2-fluoropyridine (5 g, 28.411 mmol) in DMF (30 mL) was added dropwise and the mixture was stirred at ambient temperature. Water was added and the mixture was extracted with EtOAc (2×150 mL). The combined organic extracts were washed with brine and concentrated in vacuo. Purification by flash chromatography (SiO₂, 50% EtOAc in hexanes) gave 3-((4-bromopyridin-2-yl)oxy)pyrrolidin-2-one (4.8 g, 66%) as a white solid. ¹H NMR (400 MHz, DMSO-d₆) δ 8.07 (d, J=5.4 Hz, 2H), 7.26 (q, J=1.4 Hz, 5.4 Hz, 1H), 7.19 (s, 1H), 5.53 (t, J=8.16 Hz, 1H), 3.32-3.20 (m, 2H), 2.59-2.50 (m, 1H), 2.0-1.90 (m, 1H) ppm. ESI-MS m/z calc. 255.9847, found 257.0 (M+1)⁺; Retention time: 2.97 minutes.

### Step 2:

MeI (5.7 g, 2.5 mL, 40.158 mmol) and Cs₂CO₃ (16.1 g, 49.414 mmol) were successively added to a solution of 3-((4-bromopyridin-2-yl)oxy)pyrrolidin-2-one (3.5 g, 13.614 mmol) in DMF (35 mL) at 0° C. The reaction mixture was stirred at ambient temperature for 16 h. Water was added and the mixture was extracted with EtOAc (2×150 mL). The combined organic extracts were washed with cold water (3×50 mL), brine (50 mL) and concentrated in vacuo to give 3-((4-bromopyridin-2-yl)oxy)-1-methylpyrrolidin-2-one (3.5 g, 95%) as light yellow solid. ¹H NMR (400 MHz, DMSO-d₆) δ 8.07 (d, J=5.4 Hz, 1H), 7.27 (q, J=1.3 Hz, 5.4 Hz, 1H), 7.18 (d, J=1.16 Hz, 1H), 5.58 (t, J=7.84 Hz, 1H), 3.70-3.29 (m, 2H), 2.73 (s, 3H), 2.59-2.49 (m, 1H), 1.94-1.87 (m, 1H) ppm. ESI-MS m/z calc. 270.0004, found 271.0 (M+1)⁺; Retention time: 1.54 minutes.

### Step 3:

A suspension of 3-((4-bromopyridin-2-yl)oxy)-1-methylpyrrolidin-2-one (3 g, 11.760 mmol), benzyl carbamate (2.3 g, 15.215 mmol) and Cs₂CO₃ (7.6 g, 23.326 mmol) in 1,4-dioxane (25 mL) was degassed with argon for 5 min. Pd₂(dba)₃ (861 mg, 0.9402 mmol) and XPhos (896 mg, 1.8795 mmol) were successively added and the reaction mixture was heated at 90° C. for 90 min. The reaction mixture was filtered and washed with water (50 mL), brine, (50 mL), dried (Na₂SO₄), and concentrated in vacuo. Purification by flash chromatography (SiO₂, 5% MeOH in DCM) gave benzyl (2-((1-methyl-2-oxopyrrolidin-3-yl)oxy)pyridin-4-yl)carbamate (2.5 g, 62%) as a light brown solid. ¹H NMR (400 MHz, DMSO-d₆) δ 10.2 (s, 1H), 7.97 (d, J=1.7 Hz, 1H), 7.42-7.34 (m, 5H), 7.07 (d, J=4.2 Hz, 1H), 6.92 (s, 1H), 5.52 (t, J=7.64 Hz, 1H), 5.17 (s, 2H), 3.36-3.29 (m, 2H), 2.77 (s, 3H), 2.48 (br s, 1H), 1.89-1.82 (m, 1H) ppm. ESI-MS m/z calc. 341.1376, found 342.0 (M+1)⁺; Retention time: 2.96 minutes.

### Step 4:

A solution of benzyl (2-((1-methyl-2-oxopyrrolidin-3-yl)oxy)pyridin-4-yl)carbamate (2.5 g, 7.324 mmol) in a mixture of EtOH (150 mL) and EtOAc (150 mL) was degassed with argon for 2 min. Pd/C (500 mg, 0.47 mmol) was added and the reaction mixture was stirred under a hydrogen atmosphere for 16 h. The mixture was filtered through a pad of Celite and concentrated in vacuo. The residue was triturated with diethyl ether to give 3-((4-aminopyridin-2-yl)oxy)-1-methylpyrrolidin-2-one (1.35 g, 88%) as a white solid. ¹H NMR (400 MHz, DMSO-d₆) δ 7.59 (d, J=5.7 Hz, 1H), 6.18 (q, J=1.6 Hz, 5.8 Hz, 1H), 5.95 (br s, 2H), 5.82 (d, J=1.5 Hz, 1H), 5.44 (t, J=7.8 Hz, 1H), 3.70-3.26 (m, 2H), 2.76 (s, 3H), 2.52-2.44 (m, 1H), 1.07-1.05 (m, 1H) ppm. ESI-MS m/z calc. 207.1008, found 208.0 (M+1)⁺; Retention time: 1.74 minutes.

The following intermediate was made using a method similar to that described in Intermediate DN, except that tetrahydro-2H-pyran-4-ol was used as the starting material in Step 1 and Step 2 was omitted:

| Intermediate | | | |
|---|---|---|---|
| No. | Compound Name | LC/MS | NMR (shifts in ppm) |
| DO | 2-((tetrahydro- | ESI-MS m/z calc. | ¹H NMR (400 MHz, DMSO-d₆) δ |
| | 2H-pyran-4- | 194.1055, found | 7.59 (d, J = 5.7 Hz, 1H), 6.13 |
| | yl)oxy)pyridin- | 195.0 (M + 1)⁺; | (d, J = 5.8 Hz, 1H), 5.87 (s, |
| | 4-amine | Retention time: | 2H), 5.77 (s, 1H), 5.07-5.01 (m, |
| | | 1.88 minutes | 1H), 3.84-3.79 (m, 2H), 3.47-3.41 |
| | | | (m, 2H), 1.93-1.89 (m, 2H), 1.58- |
| | | | 1.49 (m, 2H) ppm. |

The following intermediate was made using a method similar to that described in Intermediate DN, except that tetrahydrofuran-3,4-diol was used as the starting material in Step 1. Step 2 was replaced by an O-TBS protection Step, which was carried out at ambient temperature using an excess of both imidazole and TBSCl in DMF as the solvent. In Step 4, only EtOH was used as the solvent of reaction:

| Intermediate | | | |
|---|---|---|---|
| No. | Compound Name | LC/MS | NMR (shifts in ppm) |
| DP | 2-((4-((tert- | ESI-MS m/z calc. | ¹H NMR (400 MHz, DMSO-d₆) δ |
| | butyldimethylsilyl)oxy)tet- | 310.1713, found | 7.57 (d, J = 8 Hz, 1H), 6.14 (dd, |
| | rahydrofuran-3- | 311.42 (M + 1)⁺; | J = 8, 3 Hz, 1H), 5.90 (s, 2H), |
| | yl)oxy)pyridin-4-amine | Retention time: | 5.81 (s, 1H), 5.26-5.22 (m, 1H), |
| | | 1.50 minutes | 4.47-4.43 (m, 1H), 4.03-3.99 (m, |
| | | | 1H), 3.92-3.88 (m, 1H), 3.63- |
| | | | 3.59 (m, 1H), 3.56-3.52 (m, 1H), |
| | | | 0.80 (s, 9H), −0.01 (s, 3H), −0.11 |
| | | | (s, 3H) ppm. |

### Intermediate DQ

### 2-((4-((tert-butyldimethylsilyl)oxy)tetrahydrofuran-3-yl)oxy)pyrimidin-4-amine

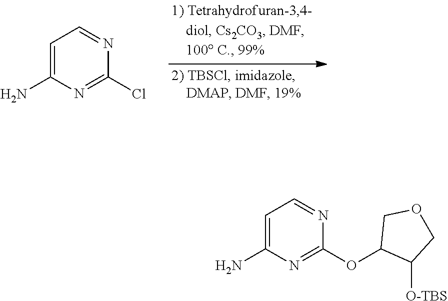

### Step 1:

Tetrahydrofuran-3,4-diol (4.4 g, 42.265 mmol) was added to a suspension of 2-chloropyrimidin-4-amine (5 g, 38.59 mmol) and Cs₂CO₃ (25.2 g, 77.344 mmol) in DMF (200 mL). The reaction mixture was heated at 100° C. for 18 h. The mixture was diluted with water (1000 mL) and extracted with 10% IPA in DCM (3×200 mL). The combined organic extracts were dried (MgSO₄), filtered and concentrated in vacuo to give 4-((4-aminopyrimidin-2-yl)oxy)tetrahydrofuran-3-ol (7.5 g, 99%). ESI-MS m/z calc. 197.08, found 198.36 (M+1)⁺; Retention time: 0.19 minutes.

### Step 2:

Imidazole (7.7 g, 113.11 mmol), DMAP (460 mg, 3.7653 mmol) and TBSCl (11.5 g, 76.299 mmol) were successively added to a solution of 4-((4-aminopyrimidin-2-yl)oxy)tetrahydrofuran-3-ol (7.5 g, 38.034 mmol) in DMF (100 mL). The reaction mixture was heated at 60° C. for 2 h. The mixture was diluted with water (500 mL) and extracted with EtOAc (2×200 mL). The combined organic extracts were dried (MgSO₄), filtered and concentrated in vacuo. Purification by flash chromatography (SiO₂, 30 to 50% EtOAc in hexanes) gave 2-((4-((tert-butyldimethylsilyl)oxy)tetrahydrofuran-3-yl)oxy)pyrimidin-4-amine (2.3 g, 19%) as white solid. ¹H NMR (400 MHz, DMSO-d₆) δ 7.82 (d, J=5.72 Hz, 1H), 6.79 (s, 2H), 6.05 (d, J=5.76 Hz, 1H), 5.26-5.22 (m, 1H), 4.51-4.48 (m, 1H), 4.05-4.01 (m, 1H), 3.92-3.89 (m, 1H), 3.69-3.66 (m, 1H), 3.54-3.51 (m, 1H), 0.75 (s, 9H), −0.01 (s, 3H), −0.13 (s, 3H) ppm. ESI-MS m/z calc. 311.1665, found 312.26 (M+1)⁺; Retention time: 1.54 minutes.

### Intermediate DR

### 1-(4-aminopyridin-2-yl)-4-methylpiperazin-2-one

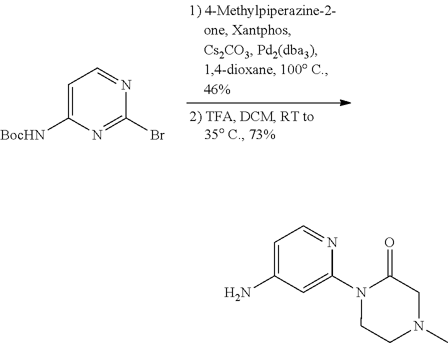

### Step 1:

A mixture of tert-butyl (2-bromopyridin-4-yl)carbamate (1.03 g, 3.77 mmol), 4-methylpiperazin-2-one (820 mg, 7.184 mmol), Pd₂(dba)₃ (181 mg, 0.198 mmol), xantphos (292 mg, 0.505 mmol) and Cs₂CO₃ (2.50 g, 7.673 mmol) were put under nitrogen (×3 vacuum/refill). 1,4-Dioxane (20 mL) was added to the mixture and the suspension was degassed by bubbling nitrogen through. The reaction mixture was heated at 100° C. for 24 h before increasing the temperature to 115° C. for a further 16 h. The mixture was cooled to ambient temperature and partitioned between water and EtOAc. The aqueous layer was separated, extracted with EtOAc (×4). The combined organic extracts were dried (MgSO₄), filtered and concentrated in vacuo. Purification by flash chromatography (SiO₂, 0 to 100% 3:1 EtOAc:EtOH containing 2% NH₄OH in heptane) gave tert-butyl (2-(4-methyl-2-oxopiperazin-1-yl)pyridin-4-yl)carbamate (530 mg, 46%) as an orange oil. ¹H NMR (500 MHz, Chloroform-d) δ 8.25 (d, J=5.7 Hz, 1H), 7.76 (d, J=2.0 Hz, 1H), 7.46 (dd, J=5.7, 2.0 Hz, 1H), 6.93 (s, 1H), 4.11-4.07 (m, 2H), 3.40 (s, 2H), 2.90 (s, 2H), 2.48 (s, 3H), 1.51 (s, 9H) ppm. ESI-MS m/z calc. 306.1692, found 307.3 (M+1)⁺; Retention time: 0.59 minutes.

### Step 2:

TFA (2 mL, 25.96 mmol) was added to a solution of tert-butyl (2-(4-methyl-2-oxopiperazin-1-yl)pyridin-4-yl)carbamate (530 mg, 1.73 mmol) in DCM (10 mL) and the reaction mixture was stirred at ambient temperature for 1 h before heating at 35° C. for 5 h. The mixture was concentrated in vacuo and azeotroped with DCM (×3) to give an orange oil. The oil was dissolved in a mixture of MeOH (3 mL) and water (2 mL) and loaded onto an SCX cartridge (5 g). The cartridge was washed with MeOH (25 mL) and the compound was released by washing the cartridge with 2 M methanolic ammonia (20 mL). The ammonia wash was concentrated in vacuo to give 1-(4-aminopyridin-2-yl)-4-methylpiperazin-2-one (260 mg, 73%) as an orange solid. ¹H NMR (500 MHz, DMSO-d₆) δ 7.85 (d, J=5.6 Hz, 1H), 6.89 (d, J=2.0 Hz, 1H), 6.34 (dd, J=5.6, 2.1 Hz, 1H), 6.06 (s, 2H), 3.75 (dd, J=6.3, 4.7 Hz, 2H), 3.10 (s, 2H), 2.66 (dd, J=6.3, 4.8 Hz, 2H), 2.25 (s, 3H) ppm. ESI-MS m/z calc. 206.11676, found 207.2 (M+1)⁺; Retention time: 0.22 minutes.

### Intermediate DS

### 3-methyl-1-(tetrahydro-2H-pyran-4-yl)-1H-pyrazol-4-amine

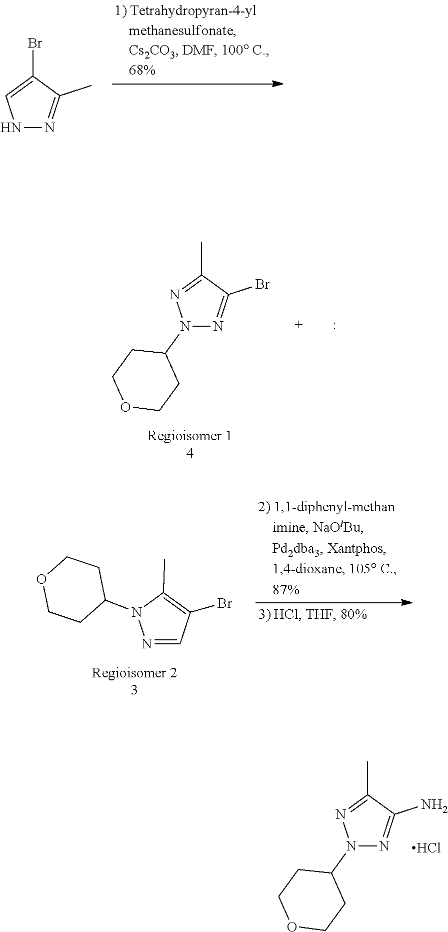

### Step 1:

In a round bottom flask, equipped with a reflux condenser, Cs₂CO₃ (2.43 g, 7.458 mmol) was added to a mixture of 4-bromo-3-methyl-1H-pyrazole (1.21 g, 7.516 mmol) and tetrahydropyran-4-yl methanesulfonate (2.03 g, 11.264 mmol) in DMF (40 mL) under an argon atmosphere. The mixture was heated at 100° C. for 23 h. Additional tetrahydropyran-4-yl methanesulfonate (0.475 g, 2.636 mmol) and Cs₂CO₃ (0.967 g, 2.968 mmol) were added and the mixture was heated at 100° C. for a further 12 h. The reaction mixture was concentrated in vacuo to remove the majority of DMF. The residue was diluted with water (30 mL) and extracted with DCM (40 mL×3). The combined organic extracts were dried (Na₂SO₄), filtered, and concentrated in vacuo. Purification by flash chromatography (40 g SiO₂, 0 to 15% EtOAc in hexanes) gave the regioisomers 4-bromo-3-methyl-1-(tetrahydro-2H-pyran-4-yl)-1H-pyrazole (779 mg, 40%) and 4-bromo-5-methyl-1-(tetrahydro-2H-pyran-4-yl)-1H-pyrazole (541 mg, 28%). 4-Bromo-3-methyl-1-(tetrahydro-2H-pyran-4-yl)-1H-pyrazole: ESI-MS m/z calc. 244.0211, found 245.2 (M+1)⁺; Retention time: 3.52 minutes. 4-Bromo-5-methyl-1-(tetrahydro-2H-pyran-4-yl)-1H-pyrazole: ESI-MS m/z calc. 244.0211, found 245.2 (M+1); Retention time: 3.52 minutes.

### Step 2:

A round bottom flask equipped with a reflux condenser was charged with 4-bromo-3-methyl-1-(tetrahydro-2H-pyran-4-yl)-1H-pyrazole (0.779 g, 3.178 mmol) and flushed with argon. 1,1-Diphenyl-methanimine (1.134 g, 1.05 mL, 6.257 mmol), xantphos (302 mg, 0.522 mmol), Pd₂(dba)₃ (247 mg, 0.27 mmol) and 1,4-dioxane (13.5 mL) were added followed by NaO^{t}Bu (1.52 g, 15.816 mmol). The system was flushed with argon. The mixture was heated at 105° C. for 2 h and 20 min. The mixture was cooled to ambient temperature, diluted with EtOAc (500 mL) and filtered through a pad of Celite. Purification by flash chromatography (40 g SiO₂, 30% EtOAc containing 1% Et₃N in hexanes containing 1% Et₃N) gave N-(3-methyl-1-(tetrahydro-2H-pyran-4-yl)-1H-pyrazol-4-yl)-1,1-diphenylmethanimine (1.01 g, 87%). ¹H NMR (500 MHz, Chloroform-d) δ 7.83-7.77 (m, 2H), 7.50 (qd, J=4.9, 1.8 Hz, 3H), 7.42-7.33 (m, 3H), 7.21 (dd, J=7.3, 2.3 Hz, 2H), 5.77 (s, 1H), 4.05-3.94 (m, 3H), 3.41 (td, J=11.9, 2.0 Hz, 2H), 2.40 (s, 3H), 1.86 (ddd, J=12.7, 4.4, 2.1 Hz, 2H), 1.68 (qd, J=12.2, 4.5 Hz, 2H) ppm. ESI-MS m/z calc. 345.1841, found 346.3 (M+1)⁺; Retention time: 4.21 minutes.

### Step 3:

A solution of N-(3-methyl-1-(tetrahydro-2H-pyran-4-yl)-1H-pyrazol-4-yl)-1,1-diphenylmethanimine (335 mg, 0.970 mmol) in THF (6.7 mL) was treated with 2 M HCl (2.4 mL, 4.8 mmol) at ambient temperature and left stirring for 2 h. The reaction mixture was concentrated in vacuo. The crude residue was partitioned between a mixture of hexanes (10 mL) and EtOAc (10 mL) and 2 M HCl (1 mL). The aqueous phase was separated and extracted twice with a mixture of hexanes (5 mL) and EtOAc (5 mL). The combined organic extracts were concentrated to in vacuo to give 3-methyl-1-(tetrahydro-2H-pyran-4-yl)-1H-pyrazol-4-amine (Hydrochloric Acid) (193 mg, 80%), as a light yellow solid. ¹H NMR (500 MHz, DMSO-d₆) δ 10.19 (br s, 3H), 7.82 (s, 1H), 4.46-4.23 (m, 1H), 3.92 (dt, J=11.7, 3.4 Hz, 2H), 3.52-3.28 (m, 2H), 2.18 (s, 3H), 1.94-1.73 (m, 4H) ppm. ESI-MS m/z calc. 181.1215, found 181.9 (M+1)⁺; Retention time: 0.42 minutes.

The following intermediate was made using a method similar to that described in Intermediate DS except that, in Step 2, 4-bromo-5-methyl-1-(tetrahydro-2H-pyran-4-yl)-1H-pyrazole (regioisomer 2 from Step 1) was used as starting material in place of 4-bromo-3-methyl-1-(tetrahydro-2H-pyran-4-yl)-1H-pyrazole (regioisomer 1 from Step 1):

| Intermediate | | | |
|---|---|---|---|
| No. | Compound Name | LC/MS | NMR (shifts in ppm) |
| DT | 5-methyl-1-(tetrahydro- | ESI-MS m/z calc. | ¹H NMR(500 MHz, |
| | 2H-pyran-4-y1)-1H- | 181.1215, found | DMSO-d₆) δ 10.11 (m, |
| | pyrazol-4-amine | 181.9 (M + 1)⁺; | 3H), 7.48 (s, 1H), 4.39 (tt, |
| | (Hydrochloric Acid) | Retention time: | J = 11.4, 4.2 Hz, 1H), |
| | | 0.42 minutes. | 4.01-3.87 (m, 2H), 3.46 |
| | | | (td, J = 12.0, 2.0 Hz, 2H), |
| | | | 2.32 (s, 3H), 1.97 (qd, J = |
| | | | 12.3, 4.6 Hz, 2H), 1.75 |
| | | | (ddd, J = 12.5, 4.3, 2.0 |
| | | | Hz, 2H) ppm. |

The following intermediates were made using a method similar to that described in Intermediate DS except that, in Step 1, 3-bromooxetane was used as the starting material in place of tetrahydropyran-4-yl methanesulfonate and the reaction was carried out in the presence of an excess of KI. The regioisomers were not separated at this point. In Step 2, the regioisomers were formed in 1:2 ratio and separated. The conditions of Step 3 were those described in Intermediate 36 Step 4:

| Intermediate | | | |
|---|---|---|---|
| No. | Compound Name | LC/MS | NMR (shifts in ppm) |
| DU | 3-methyl-1-(oxetan- | ESI-MS m/z calc. | ¹H NMR (500 MHz, |
| | 3-y1)-1H-pyrazol- | 153.0902, found | DMSO-d₆) δ 7.08 (s, 1H), |
| | 4-amine | 154.0 (M + 1)⁺; | 5.27 (tt, J = 7.7, 6.5 Hz, |
| | | Retention time: | 1H), 4.83-4.75 (m, 4H), |
| | | 0.24 minutes | 3.74 (s, 2H), 2.03 (s, 3H) |
| | | | ppm. |
| DV | 5-methyl-1-(oxetan- | ESI-MS m/z calc. | |
| | 3-y1)-1H-pyrazol- | 153.0902, found | |
| | 4-amine | 154.5 (M + 1)⁺; | |
| | | Retention time: | |
| | | 0.71 minutes | |

### Intermediate DW

### 3-methylimidazo[1,5-a]pyridin-6-amine

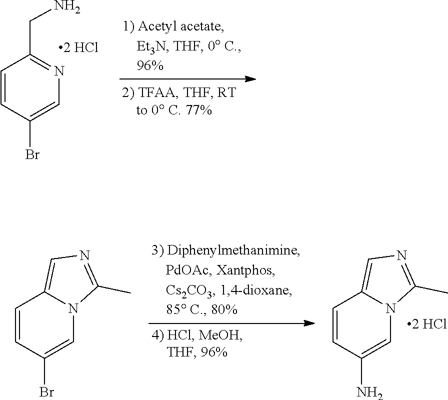

### Step 1:

A solution of (5-bromopyridin-2-yl)methanamine (6.5 g, 34.057 mmol) and Et₃N (4.5012 g, 6.2 mL, 44.483 mmol) in THF (70 mL) was stirred at ambient temperature for 5 min then cooled to 0° C. Acetyl acetate (3.803 g, 3.7 mL, 35.391 mmol) was added slowly. The reaction mixture was warmed up to ambient temperature and stirred overnight. MeOH (30 mL) was added, and the mixture was stirred for 5 min then concentrated in vacuo. The residue was dissolved in DCM (400 mL) and washed with water (4×30 mL) and brine (80 mL), dried (Na₂SO₄), filtered and concentrated in vacuo to give N-((4-bromopyridin-2-yl)methyl)acetamide (7.49 g, 96%) as an off-white solid. ¹H NMR (300 MHz, Chloroform-d) δ 8.59 (d, J=2.3 Hz, 1H), 7.78 (dd, J=8.5, 2.3 Hz, 1H), 7.18 (d, J=8.2 Hz, 1H), 6.59 (br s, 1H), 4.51 (d, J=5.0 Hz, 2H), 2.07 (s, 3H) ppm. ESI-MS m/z calc. 227.9898, found 229.1 (M+1)⁺; Retention time: 1.32 minutes.

### Step 2:

A solution of N-((4-bromopyridin-2-yl)methyl)acetamide (7.45 g, 32.522 mmol) and Et₃N (3.92 g, 5.4 mL, 38.743 mmol) in THF (90 mL) was stirred at ambient temperature for 5 min, then cooled to 0° C. TFAA (7.404 g, 4.9 mL, 35.251 mmol) was added dropwise. The reaction mixture was warmed up to ambient temperature, then stirred for an additional 30 min. The white solid was filtered and treated with 1 N aqueous NaOH solution (80 mL) and DCM (100 mL). The heterogeneous biphasic mixture was vigorously stirred for 5 min and the layers were separated. The aqueous layer was extracted with DCM (2×90 mL). The combined organic layers were washed with water (2×50 mL), brine (100 mL) dried (Na₂SO₄), filtered and concentrated in vacuo to give 6-bromo-3-methylimidazo[1,5-a]pyridine (5.280 g, 77%) as pale yellow solid. ¹H NMR (300 MHz, Chloroform-d) δ 7.83 (s, 1H), 7.35 (s, 1H), 7.31 (d, J=9.4 Hz, 1H), 6.69 (d, J=9.4 Hz, 1H), 2.63 (s, 3H) ppm. ESI-MS m/z calc. 209.9793, found 211.1 (M+1)⁺; Retention time: 0.78 minutes.

### Step 3:

A mixture of 6-bromo-3-methyl-imidazo[1,5-a]pyridine (3.74 g, 17.702 mmol) and diphenylmethanimine (3.996 g, 3.7 mL, 22.049 mmol) in 1,4-dioxane (75 mL) was degassed by bubbling nitrogen through the solution for 15 min. Cs₂CO₃ (11.5 g, 35.296 mmol), xantphos (1.1 g, 1.901 mmol) and PdOAc (220 mg, 0.98 mmol) were successively added and the resulting mixture was stirred overnight at 85° C. The reaction was cooled to ambient temperature and EtOAc (75 mL) was added. The mixture was filtered, and the filtrate was concentrated in vacuo. Purification by flash chromatography (120 g SiO₂, 40 to 100% EtOAc in heptane) gave N-(3-methylimidazo[1,5-a]pyridin-6-yl)-1,1-diphenylmethanimine (4.41 g, 80%) as a yellow solid. ¹H NMR (300 MHz, Chloroform-d) δ 7.79-7.69 (m, 2H), 7.54-7.46 (m, 1H), 7.46-7.38 (m, 2H), 7.38-7.29 (m, 3H), 7.24-7.12 (m, 5H), 6.21 (dd, J=9.4, 1.5 Hz, 1H), 2.48 (s, 3H) ppm. ESI-MS m/z calc. 311.1422, found 312.2 (M+1)⁺; Retention time: 1.74 minutes.

### Step 4:

To a solution of N-(3-methylimidazo[1,5-a]pyridin-6-yl)-1,1-diphenylmethanimine (4.41 g, 14.163 mmol) in THF (20 mL) and MeOH (50 mL) at ambient temperature was added dropwise 3 M HCl (14 mL, solution in MeOH, 42 mmol). The reaction mixture was stirred at ambient temperature for 40 min then the solvents were removed in vacuo (35° C.). The residue was sonicated and triturated in diethyl ether (100 mL). The solid was filtered to give 3-methylimidazo[1,5-a]pyridin-6-amine (2 Hydrochloric acid) (3.017 g, 96%) as an off-white solid. ¹H NMR (300 MHz, DMSO-d₆) δ 7.87 (s, 1H), 7.61 (d, J=10.0 Hz, 1H), 7.33 (s, 1H), 6.88 (dd, J=9.7, 1.5 Hz, 1H), 2.68 (s, 3H) ppm; NH₂ amine and HCl not observed. ESI-MS m/z calc. 147.0796, found 148.2 (M+1)⁺; Retention time: 0.36 minutes.

The following intermediate was made using a method similar to that described in Intermediate DW except that, in Step 1, (4-bromopyridin-2-yl)methanamine was used as the starting material in place of (5-bromopyridin-2-yl)methanamine. In Step 2, DCM was used as the solvent in place of THF and in Step 4, 1,4-dioxane was used as solvent in place of THF:

| Intermediate | | | |
|---|---|---|---|
| No. | Compound Name | LC/MS | NMR (shifts in ppm) |
| DX | 3-methylimidazo[1,5- | ESI-MS m/z calc. | ¹H NMR (300 MHz, |
| | a]pyridin-7-amine | 147.0796, found | DMSO-d₆) δ 8.18 (d, J = |
| | (Hydrochloride salt) | 166.3 (M + 19)⁺; | 7.6 Hz, 1H), 7.34 (s, 1H), |
| | | Retention time: | 7.08-6.46 (m, 4H), 6.36 |
| | | 0.29 minutes. | (d, J = 1.8 Hz, 1H), |
| | | | 2.74 (s, 3H) ppm. |

### Intermediate DY

### tert-butyl (3-(4-aminopyridin-2-yl)oxetan-3-yl)carbamate

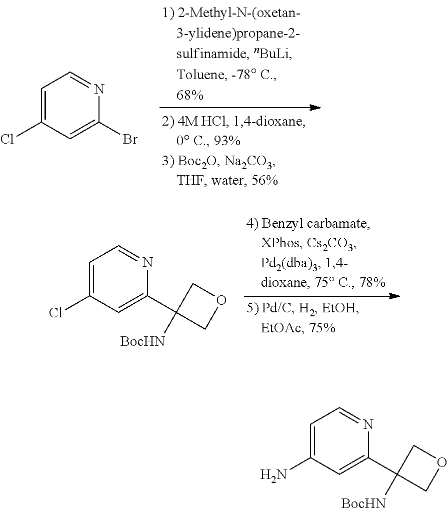 [SMILES: Clc1ccnc(Br)c1 ; CC(C)(C)OC(=O)NC1(c2cc(Cl)ccn2)COC1 ; CC(C)(C)OC(=O)NC1(c2cc(N)ccn2)COC1]

### Step 1:

ⁿBuLi (24 mL, 2.3 M solution in hexanes, 55.2 mmol) was added dropwise to a solution of 2-bromo-4-chloro-pyridine (10 g, 51.964 mmol) in toluene (100 mL) at −78° C. and the reaction mixture was stirred at −78° C. for 2 h. A solution of 2-methyl-N-(oxetan-3-ylidene)propane-2-sulfinamide (11 g, 62.767 mmol) in toluene (60 mL) was added dropwise over 15 min and the mixture was stirred at −78° C. for 15 min. The reaction mixture was slowly warmed to ambient temperature and stirred for 2 h. The reaction mixture was quenched with a saturated NH₄Cl solution (100 mL) and extracted with EtOAc (3×150 mL). The combined organic extracts were washed with water, brine and concentrated in vacuo. Purification by flash chromatography (SiO₂, 50% EtOAc in hexanes) gave N-(3-(4-chloropyridin-2-yl)oxetan-3-yl)-2-methylpropane-2-sulfinamide (11 g, 68%). ¹H NMR (400 MHz, DMSO-d₆) δ 8.61 (d, J=5.32 Hz, 1H), 7.71 (d, J=1.7 Hz, 1H), 7.52-7.51 (m, 1H), 6.50 (s, 1H), 5.06 (d, J=6.2 Hz, 1H), 4.93 (d, J=6.3 Hz, 1H), 4.88 (d, J=6.3 Hz, 1H), 4.77 (d, J=6.2 Hz, 1H), 1.16 (s, 9H) ppm. ESI-MS m/z calc. 288.0699, found 289.0 (M+1)⁺; Retention time: 2.75 minutes.

### Step 2:

4 M HCl (25 mL, solution in 1,4-dioxane, 100 mmol) was added dropwise to a solution of N-(3-(4-chloropyridin-2-yl)oxetan-3-yl)-2-methylpropane-2-sulfinamide (10 g, 34.627 mmol) in 1,4-dioxane (100 mL) at 0° C. The reaction mixture was stirred at ambient temperature for 3 h and then concentrated in vacuo. The crude was triturated with Et₂O (3×50 mL) to give 3-(4-chloropyridin-2-yl)oxetan-3-amine (Hydrochloric Acid) (7.5 g, 93%). ¹H NMR (400 MHz, DMSO-d₆) δ 9.39 (br s, 2H), 8.66 (d, J=5.36 Hz, 1H), 8.05 (s, 1H), 7.67-7.65 (m, 1H), 5.44 (br s, 1H), 4.97-4.90 (m, 4H) ppm. ESI-MS m/z calc. 184.0403, found 185.0 (M+1)⁺; Retention time: 1.84 minutes.

### Step 3:

2 M Na₂CO₃ (265 mL, 530 mmol) was added to a stirred solution of 3-(4-chloropyridin-2-yl)oxetan-3-amine (16 g, 86.663 mmol) in THF (200 mL) and pH was adjusted to 10-11. Boc₂0 (57 g, 60 mL, 261.17 mmol) was added dropwise and the reaction mixture was stirred at ambient temperature overnight. The reaction mixture was quenched with water and extracted with EtOAc. The organic extracts were dried (MgSO₄), filtered and concentrated in vacuo. Purification by flash chromatography gave tert-butyl (3-(4-chloropyridin-2-yl)oxetan-3-yl)carbamate (14 g, 56%) as an off white solid. ¹H NMR (400 MHz, Chloroform-d) δ 8.50 (d, J=4.12 Hz, 1H), 7.70 (s, 1H), 6.03 (s, 1H), 5.17 (s, 2H), 4.89 (s, 2H), 1.45 (s, 9H) ppm; NH amine not observed. ESI-MS m/z calc. 284.0928, found 285.0 (M+1)⁺; Retention time: 1.57 minutes.

### Step 4:

Benzyl carbamate (2.5 g, 16.538 mmol) and XPhos (893 mg, 1.873 mmol) were successively added to a solution of tert-butyl (3-(4-chloropyridin-2-yl)oxetan-3-yl)carbamate (3.5 g, 12.292 mmol) in 1,4-dioxane (35 mL) and the mixture was degassed with argon for 5 min. Cs₂CO₃ (7 g, 21.484 mmol) and Pd₂(dba)₃ (875 mg, 0.956 mmol) were added and the mixture was heated at 75° C. for 4 h. The reaction mixture was filtered through a pad of Celite and concentrated in vacuo. Purification by flash chromatography gave tert-butyl (3-(4-(((benzyloxy)carbonyl)amino)pyridin-2-yl)oxetan-3-yl)carbamate (4 g, 78%) as off white solid. ¹H NMR (400 MHz, DMSO-d₆) δ 10.25 (s, 1H), 8.41 (d, J=5.44 Hz, 1H), 8.12 (s, 1H), 7.55 (s, 1H) 7.41-7.31 (m, 5H), 5.14 (s, 2H), 4.82 (d, p=5.92 Hz, 2H), 4.62 (d, J=5.92 Hz 2H), 1.37 (s, 9H) ppm. ESI-MS m/z calc. 399.1794, found 400.0 (M+1)⁺; Retention time: 3.18 minutes.

### Step 5:

A solution of tert-butyl (3-(4-(((benzyloxy)carbonyl)amino)pyridin-2-yl)oxetan-3-yl)carbamate (700 mg, 1.75 mmol) in EtOH (15 mL) and EtOAc (15 mL) was degassed with argon. Pd/C (95 mg, 0.4698 mmol) was added and the reaction mixture was stirred under a hydrogen atmosphere for 16 h. The mixture was filtered through a pad of Celite and the filtrates were concentrated in vacuo. Purification by flash chromatography (SiO₂, 900 EtOAc in hexanes) gave tert-butyl (3-(4-aminopyridin-2-yl)oxetan-3-yl)carbamate (350 mg, 75) as a white solid. H NMR (400 μMHz, DMSO-d₆) δ 8.02-7.95 (m, 2H), 6.50 (s, 1H), 6.36 (t, J=1.52 Hz, 1H), 6.03 (s, 2H), 4.81 (d, (=5.7 Hz, 2H), 4.63 (d, J=4.84 Hz, 2H), 1.40 (s, 9H) ppm. ESI-MS m/z calc. 265.1426, found 266.0 (M+1)⁺; Retention time: 2.7 minutes. 0015631 The following intermediates were made using a method similar to that described in Intermediate DY, except that different starting materials were used in Step 1:

| Intermediate | | | |
|---|---|---|---|
| No. | Compound Name | LC/MS | NMR (shifts in ppm) |
| DZ | tert-butyl (3-(4-amino- | ESI-MS m/z calc. | ¹H NMR (400 MHz, DMSO-d₆) δ |
| | 5-fluoropyridin-2- | 283.1332, found | 8.10 (s, 1H), 8.01 (s, 1H), 6.72- |
| | yl)oxetan-3- | 284.16 (M + 1)⁺; | 6.71 (m, 1H), 6.21 (s, 2H), 4.85- |
| | yl)carbamate | Retention time: | 4.80 (m, 2H), 4.64-4.61 (m, 2H), |
| | | 1.15 minutes | 1.39 (s, 9H) ppm. |
| EA | tert-butyl (3-(4- | ESI-MS m/z calc. | ¹H NMR (400 MHz, DMSO-d₆) δ |
| | aminopyridin-2- | 279.1583, found | 7.89 (d, J = 5.5 Hz, 1H), 7.41 (s, |
| | yl)tetrahydrofuran-3- | 280.1 (M + 1)⁺; | 1H), 6.52 (s, 1H) , 6.30 (d, J = 4 |
| | yl)carbamate | Retention time: | Hz, 1H), 5.95 (s , 2H) , 4.06-3.94 |
| | | 1.97 minutes | (m, 2H), 3.84 (t, J = 7.28 Hz, 2H), |
| | | | 2.34-2.25 (m, 2H), 1.38 (s, 9H) |
| | | | ppm. |
| EB | tert-butyl (3-(4-amino- | ESI-MS m/z calc. | ¹H NMR (400 MHz, DMSO-d₆) δ |
| | 5-fluoropyridin-2- | 297.1489, found | 7.98 (s, 1H), 7.47 (s, 1H), 6.73 (d, |
| | yl)tetrahydrofuran-3- | 298.11 (M + 1)⁺; | J = 4 Hz, 1H), 6.13 (s, 2H), 4.02- |
| | yl)carbamate | Retention time: | 3.96 (m, 2H), 3.85-3.81 (m, 2H), |
| | | 1.17 minutes | 2.32-2.25 (m, 2H), 1.37 (s, 9H) |
| | | | ppm. |
| EC | tert-butyl (3-(5- | ESI-MS m/z calc. | ¹H NMR (400 MHz, DMSO-d₆) δ |
| | aminopyridin-2- | 265.1426, found | 7.96-7.95 (m, 2H), 7.0 (d, J = |
| | yl)oxetan-3- | 266.2 (M + 1)⁺; | 8.44 Hz, 1H), 6.90 (s, 1H) 5.25 (s, |
| | yl)carbamate | Retention time: | 2H), 4.80 (d, J = 5.76 Hz, 2H), |
| | | 0.69 minutes | 4.64 (d, J = 5.24 Hz, 2H), 1.38 (s, |
| | | | 9H) ppm. |

### Intermediate ED

### tert-butyl (3-(4-aminopyrimidin-2-yl)oxetan-3-yl)carbamate

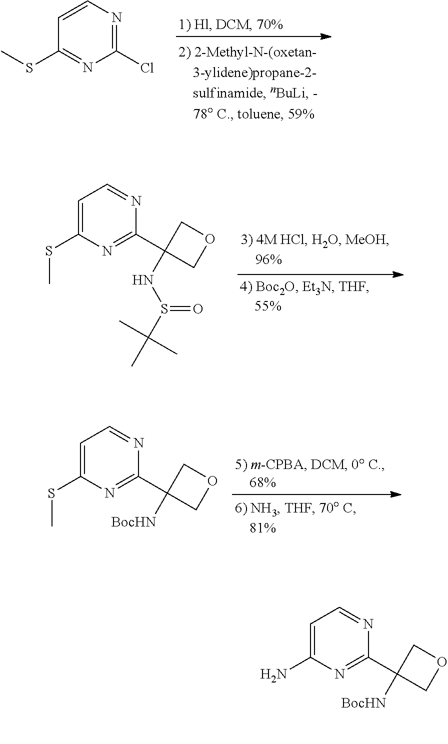

### Step 1:

HI (17 mL, 57% w/v solution in water, 75.755 mmol) was added to a stirred solution of 2-chloro-4-methylthiopyrimidine (10 g, 62.257 mmol) in DCM at ambient temperature (100 mL) and the mixture was stirred for 18 h. The reaction mixture was filtered and the solid was partitioned between a saturated NaHCO₃ solution and ethyl acetate (400 mL). The organic extract was separated, dried (Na₂SO₄) and concentrated in vacuo. Purification by flash chromatography (SiO₂, 5 to 10% EtOAc in hexanes) gave 2-iodo-4-methylsulfanyl-pyrimidine (11 g, 70%). ¹H NMR (400 MHz, DMSO-d₆) δ 8.18 (d, J=5.48 Hz, 1H), 7.47 (d, J=5.52 Hz, 1H), 2.50 (s, 3H) ppm.

### Step 2:

ⁿBuLi (11 mL, 2 M solution in hexanes, 22 mmol) was added dropwise to a stirred solution of 2-iodo-4-methylsulfanyl-pyrimidine (5 g, 19.835 mmol) in toluene (50 mL) at −78° C. The reaction mixture was stirred at −78° C. for 30 min. A solution of 2-methyl-N-(oxetan-3-ylidene)propane-2-sulfinamide (5 g, 28.531 mmol) in toluene (10 mL) was added and the mixture was stirred for 10 min at -78° C. The mixture was quenched by addition of saturated NH₄Cl solution (100 mL). The mixture was extracted with EtOAc (200 mL×2). The combined organic extracts were dried (Na₂SO₄), filtered and concentrated in vacuo. Purification by flash chromatography (SiO₂, 50 to 90% EtOAc in hexanes) gave 2-methyl-N-(3-(4-(methylthio)pyrimidin-2-yl)oxetan-3-yl)propane-2-sulfinamide (3.5 g, 59%). ¹H NMR (400 MHz, DMSO-d₆) δ 8.53 (d, J=5.4 Hz, 1H), 7.40 (d, J=5.5 Hz, 1H), 6.29 (s, 1H), 5.13 (d, J=6.2 Hz, 1H), 4.96 (d, J=6.4 Hz, 1H), 4.91 (d, J=6.3 Hz, 1H), 4.81 (d, J=6.3 Hz, 1H) 2.56 (s, 3H), 1.11 (s, 9H) ppm. ESI-MS m/z calc. 301.09, found 302.0 (M+1)⁺; Retention time: 2.53 minutes.

### Step 3:

6 M HCl (10 mL, 60 mmol) was added dropwise to a solution of 2-methyl-N-(3-(4-(methylthio)pyrimidin-2-yl)oxetan-3-yl)propane-2-sulfinamide (3.5 g, 11.611 mmol) in MeOH (20 mL) at 0° C. The reaction mixture was stirred at ambient temperature for 1 h. A saturated NH₄Cl solution (100 mL) was added and the mixture was extracted with DCM (2×200 mL). The combined organic extracts were dried (Na₂SO₄), filtered and concentrated in vacuo to give 3-(4-(methylthio)pyrimidin-2-yl)oxetan-3-amine (2.2 g, 96%). ¹H NMR (400 MHz, DMSO-d₆) δ 8.51 (d, J=5.44 Hz, 1H), 7.36 (d, J=5.52 Hz, 1H), 4.93 (d, J=5.72 Hz, 2H), 4.56 (d, J=5.64 Hz, 2H), 3.72 (s, 2H), 2.57 (s, 3H) ppm. ESI-MS m/z calc. 197.062, found 198.0 (M+1)⁺; Retention time: 1.54 minutes.

### Step 4:

Et₃N (0.6 mL, 4.305 mmol) was added to a stirred solution of 3-(4-(methylthio)pyrimidin-2-yl)oxetan-3-amine (300 mg, 1.521 mmol) in THF (5 mL) and the reaction mixture was stirred for 5 min. Boc₂0 (665 mg, 3.047 mmol) was added and the reaction was stirred at ambient temperature for 2 h. A saturated NH₄Cl solution (100 mL) was added and the mixture was extracted with EtOAc (2×200 mL). The combined organic extracts were dried (Na₂SO₄), filtered and concentrated in vacuo. Purification by flash chromatography (SiO₂, 30 to 50% EtOAc in hexanes) gave tert-butyl (3-(4-(methylthio)pyrimidin-2-yl)oxetan-3-yl)carbamate (250 mg, 55%). ¹H NMR (400 MHz, DMSO-d₆) δ 8.53 (d, J=5.16 Hz, 1H), 8.01 (s, 1H), 7.34 (d, J=5.2 Hz, 1H), 4.93 (d, J=6 Hz, 2H), 4.83 (d, J=5.4 Hz, 2H), 2.56 (s, 3H), 1.37-1.06 (m, 9H) ppm. ESI-MS m/z calc. 297.1147, found 298.0 (M+1)⁺; Retention time: 2.92 minutes.

### Step 5:

m-CPBA (840 mg, 4.867 mmol) was added to a stirred solution of tert-butyl (3-(4-(methylthio)pyrimidin-2-yl)oxetan-3-yl)carbamate (1.4 g, 4.7 mmol) in DCM (25 mL) at 0 to 5° C. The reaction mixture was stirred at 0 to 5° C. for 1 h. A saturated NH₄Cl solution (100 mL) was added and the mixture was extracted with DCM (2×200 mL). The combined organic extracts were dried (Na₂SO₄), filtered and concentrated in vacuo. Purification by flash chromatography (SiO₂, 2 to 5% MeOH in EtOAc) gave tert-butyl (3-(4-(methylsulfinyl)pyrimidin-2-yl)oxetan-3-yl)carbamate (1 g, 68%). ¹H NMR (400 MHz, DMSO-d₆) δ 9.17 (d, J=4.8 Hz, 1H), 8.15 (s, 1H), 7.92 (d, J=4.84 Hz, 1H), 5.01-4.88 (m, 2H), 4.73-4.70 (m, 2H), 2.86 (s, 3H), 1.37-1.06 (m, 9H) ppm. ESI-MS m/z calc. 313.109, found 314.0 (M+1)⁺; Retention time: 2.14 minutes.

### Step 6:

A solution of tert-butyl (3-(4-(methylsulfinyl)pyrimidin-2-yl)oxetan-3-yl)carbamate (1 g, 3.191 mmol) in THF (30 mL) at 0° C. was purged the NH₃ gas for 5 min and then heated at 70° C. for 24 h. The reaction mixture was concentrated in vacuo. Purification by flash chromatography (SiO₂, 5 to 10% EtOAc in MeOH) gave tert-butyl (3-(4-aminopyrimidin-2-yl)oxetan-3-yl)carbamate (700 mg, 81%) as white solid. ¹H NMR (400 MHz, DMSO-d₆) δ 8.07 (d, J=5.28 Hz, 1H), 7.70-7.45 (m, 1H), 6.84 (s, 2H), 6.30 (d, J=5.72 Hz, 1H) 4.87 (d, J=5.28 Hz, 2H), 4.66-4.55 (m, 2H), 1.37-1.12 (m, 9H) ppm. ESI-MS m/z calc. 266.1379, found 267.0 (M+1)⁺; Retention time: 1.15 minutes.

### Intermediate EE

### tert-butyl 3-(4-aminopyridin-2-yl)morpholine-4-carboxylate

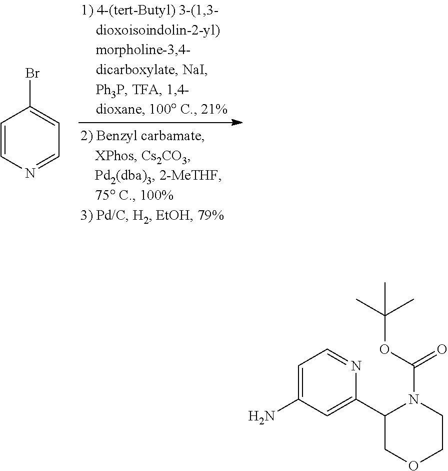 [SMILES: Brc1ccncc1 ; CC(C)(C)OC(=O)N1CCOCC1c1cc(N)ccn1]

### Step 1:

An oven dried vial (pre-dried in drying pistol at 100° C., 10 mbar for 30 min) was charged with 4-(tert-butyl) 3-(1,3-dioxoisoindolin-2-yl) morpholine-3,4-dicarboxylate (1.16 g, 2.959 mmol), NaI (31 mg, 0.206 mmol) and Ph₃P (108 mg, 0.41 mmol). The vial was sealed, evacuated and backfilled with argon (×3). 1,4-Dioxane (15 mL), 4-bromopyridine (324 mg, 2.051 mmol) and TFA (160 μL, 2.077 mmol) were successively added via syringe and the mixture was irradiated in a Merck photoreactor 2 for 22 h. The reaction mixture was poured onto water (100 mL) and extracted with EOAc (3×50 mL). The combined organic phases were dried (MgSO₄), filtered and concentrated in vacuo. Purification by flash chromatography (SiO₂, 3:1 EtOH:EtOAC containing 2% NH₄OH in heptane) gave tert-butyl 3-(4-bromopyridin-2-yl)morpholine-4-carboxylate (165 mg, 21%). ESI-MS m/z calc. 342.0579, found 287.4 (M-^{t}Bu+1)⁺; Retention time: 0.86 minutes.

### Step 2:

A reaction vial was charged with tert-butyl 3-(4-bromopyridin-2-yl)morpholine-4-carboxylate (165 mg, 0.452 mmol), benzyl carbamate (82 mg, 0.543 mmol), Pd₂(dba)₃ (4.1 mg, 0.004 mmol), XPhos (8.6 mg, 0.018 mmol), Cs₂CO₃ (207 mg, 0.635 mmol) and 2-MeTHF (2.5 mL). The mixture was flushed with nitrogen and heated at 80° C. for 16 h. Further amounts of benzyl carbamate (40 mg, 0.264 mmol), Pd₂(dba)₃ (4.1 mg, 0.004 mmol) and XPhos (8.6 mg, 0.018 mmol) were added and the reaction mixture heated at 80° C. for 21 h. The mixture was washed with water (2 mL) and the aqueous phase was extracted with EOAc (3 mL). The combined organic extracts were dried (MgSO₄), filtered and concentrated in vacuo. Purification by flash chromatography (SiO₂, 10 to 100% EtOAc in heptane) gave tert-butyl 3-(4-(((benzyloxy)carbonyl)amino)pyridin-2-yl)morpholine-4-carboxylate (209 mg, 100%). ESI-MS m/z calc. 413.195, found 414.7 (M+1)⁺; 412.6 (M−1)⁻; Retention time: 0.88 minutes.

### Step 3:

A solution of tert-butyl 3-(4-(((benzyloxy)carbonyl)amino)pyridin-2-yl)morpholine-4-carboxylate (209 mg, 0.45 mmol) in EtOH (7.5 mL) was degassed with nitrogen. Pd/C (67 mg of 10% w/w, 0.063 mmol) was added and the reaction mixture was stirred under a hydrogen atmosphere for 2 h. The mixture was filtered through a pad of Celite and the filtrates were concentrated in vacuo. Purification by flash chromatography (SiO₂, 0 to 80% of 3:1 EtOH:EtOAc containing 2% NH₄OH in heptane) gave tert-butyl 3-(4-aminopyridin-2-yl)morpholine-4-carboxylate (99.5 mg, 79%) as a colourless oil. ESI-MS m/z calc. 279.1583, found 280.6 (M+1)⁺; Retention time: 0.51 minutes.

### Intermediate EF

### 5-fluoro-2-vinyl-pyridin-4-amine

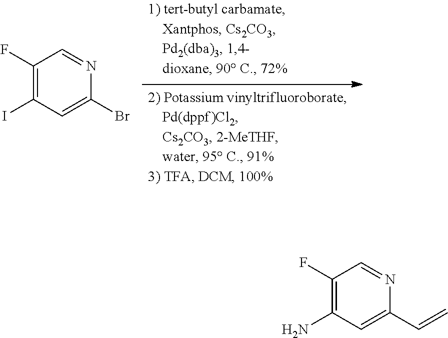

### Step 1:

tert-Butyl carbamate (4 g, 34.15 mmol), 2-bromo-5-fluoro-4-iodo-pyridine (10 g, 33.13 mmol), Cs₂CO₃ (21.6 g, 66.29 mmol), xantphos (147 mg, 0.254 mmol) and Pd₂(dba)₃ (156 mg, 0.1704 mmol) were placed in reaction vessel under nitrogen. 1,4-Dioxane (75 mL) was added and the mixture was degassed for 5 min. The reaction mixture was heated at 90° C. for 3 days. The reaction mixture was partitioned between EtOAc and water. The organic extracts were dried (MgSO₄), filtered and concentrated in vacuo. Purification by flash chromatography (220 g SiO₂, 0 to 70% EtOAc in hexanes) gave tert-butyl (2-bromo-5-fluoropyridin-4-yl)carbamate (6.9 g, 72%) as a white solid. ¹H NMR (400 MHz, Chloroform-d) δ 8.35 (d, J=5.8 Hz, 1H), 8.14 (d, J=2.2 Hz, 1H), 6.94 (s, 1H), 1.56 (s, 9H) ppm. ESI-MS m/z calc. 290.006, found 291.3 (M+1)⁺; Retention time: 2.13 minutes.

### Step 2:

Pd(dppf)Cl₂·DCM (520 mg, 0.636 mmol) and Cs₂CO₃ (18 g, 55.25 mmol) were successively added to a solution of tert-butyl (2-bromo-5-fluoropyridin-4-yl)carbamate (7700 mg, 26.45 mmol) and potassium vinyltrifluoroborate (5 g, 37.33 mmol) in a mixture of 2-MeTHF (150 mL) and water (8 mL). The reaction mixture was heated at 95° C. for 18 h. The reaction mixture was diluted with water (100 mL) and extracted with EtOAc (3×100 mL). The combined organic extracts were dried (MgSO₄), filtered and concentrated in vacuo. Purification by flash chromatography (SiO₂, 0 to 100% EtOAc in heptane) gave tert-butyl (5-fluoro-2-vinylpyridin-4-yl)carbamate (5750 mg, 91%) as an white solid. ¹H NMR (400 MHz, DMSO-d₆) δ 9.65 (d, J=1.6 Hz, 1H), 8.40 (d, J=2.8 Hz, 1H), 7.98 (d, J=6.6 Hz, 1H), 6.76 (dd, J=17.4, 10.7 Hz, 1H), 6.08 (dd, J=17.4, 1.6 Hz, 1H), 5.42 (ddd, J=10.7, 1.6, 0.7 Hz, 1H), 1.50 (s, 9H) ppm. ESI-MS m/z calc. 238.11, found 183.8 (M−56)⁺; Retention time: 1.36 minutes.

### Step 3:

A Boc deprotection Step of tert-butyl (5-fluoro-2-vinylpyridin-4-yl)carbamate was carried out according to General Method I to give 5-fluoro-2-vinylpyridin-4-amine (Trifluoroacetate salt) (2.4 g, 100%). ¹H NMR (400 MHz, Chloroform-d) δ 8.26 (dd, J=5.3, 2.1 Hz, 1H), 7.05 (dd, J=7.6, 1.0 Hz, 1H), 6.73 (dd, J=17.6, 11.2 Hz, 1H), 6.26 (d, J=17.6 Hz, 1H), 6.09 (s, 2H), 5.85 (d, J=11.2 Hz, 1H) ppm. ESI-MS m/z calc. 138.059, found 139.1 (M+1)⁺; Retention time: 0.39 minutes.

### Intermediate EG

### tert-butyl 2-(4-amino-5-fluoropyridin-2-yl)morpholine-4-carboxylate

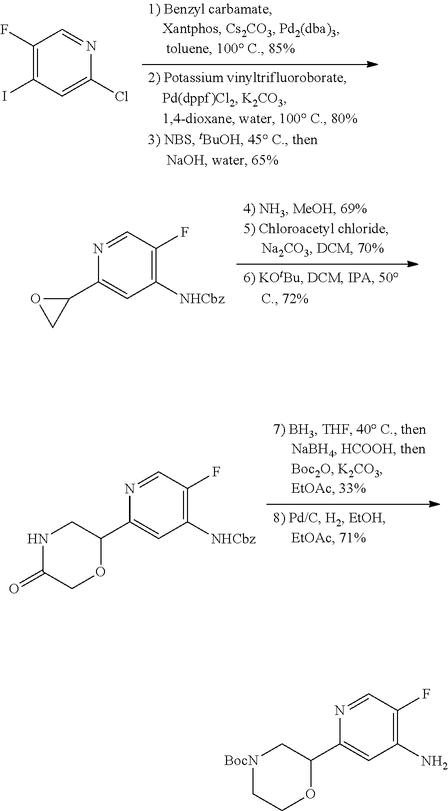 [SMILES: Fc1cnc(Cl)cc1I ; O=C(Nc1cc(C2CO2)ncc1F)OCc1ccccc1 ; O=C1COC(c2cc(NC(=O)OCc3ccccc3)c(F)cn2)CN1 ; CC(C)(C)OC(=O)N1CCOC(c2cc(N)c(F)cn2)C1]

### Step 1:

Benzyl carbamate (9.4 g, 62.185 mmol) and Cs₂CO₃ (38 g, 116.63 mmol) were successively added to a stirred solution of 2-chloro-5-fluoro-4-iodo-pyridine (15 g, 58.268 mmol) in toluene (375 mL) and the mixture was purged with argon. Pd₂(dba)₃ (1.07 g, 1.169 mmol) and Xantphos (1.02 g, 1.762 mmol) were added and the reaction mixture was heated at 100° C. for 5 h. The reaction mixture was filtered through a pad of Celite and washed with EtOAc (500 mL). The filtrates were washed with water (150 mL), brine (150 mL), dried (MgSO₄), filtered and concentrated in vacuo. Purification by flash chromatography (SiO₂, 5 to 10% EtOAc in hexanes) gave benzyl (2-chloro-5-fluoropyridin-4-yl)carbamate (15.5 g, 85%) as a white solid. ¹H NMR (400 MHz, Chloroform-d) δ 8.20 (d, J=5.6 Hz, 1H), 8.13 (s, 1H), 7.38 (d, J=12.5 Hz, 5H), 7.11 (br s, 1H), 5.24 (s, 2H) ppm. ESI-MS m/z calc. 280.04, found 281.0 (M+1)⁺; Retention time: 3.47 minutes.

### Step 2:

Pd(dppf)Cl₂·DCM (4.5 g, 5.510 mmol) and K₂CO₃ (20 g, 144.71 mmol) were successively added to a solution of benzyl (2-chloro-5-fluoropyridin-4-yl)carbamate (15.5 g, 55.223 mmol) and potassium vinyltrifluoroborate (15 g, 111.98 mmol) in a mixture of 1,4-dioxane (160 mL) and water (16 mL). The reaction mixture was purged with argon and heated at 100° C. for 5 h. The reaction mixture was filtered through a pad of Celite and washed with EtOAc (500 mL). The filtrates were washed with water (100 mL), brine (100 mL), dried (MgSO₄), filtered and concentrated in vacuo. Purification by flash chromatography (SiO₂, 10 to 15% EtOAc in hexanes) gave benzyl (5-fluoro-2-vinylpyridin-4-yl)carbamate (13 g, 80%) as a white solid. ¹H NMR (400 MHz, Chloroform-d) δ 8.31 (s, 1H), 8.21 (s, 1H), 7.40 (s, 5H), 7.08 (brs, 1H), 6.78-6.71 (m, 1H), 6.12 (d, J=17.6 Hz, 1H), 5.44 (d, J=10.4 Hz, 1H), 5.24 (s, 2H) ppm. ESI-MS m/z calc. 272.0961, found 273.0 (M+1)⁺; Retention time: 3.4 minutes.

### Step 3:

The epoxide formation Step 3 was carried out according to General Method T Step 1 to give benzyl (5-fluoro-2-(oxiran-2-yl)pyridin-4-yl)carbamate (130 mg, 65%). ¹H NMR (400 MHz, DMSO-d₆) δ 10.21 (s, 1H), 8.42 (d, J=2.64 Hz, 1H), 7.88 (d, J=6.48 Hz, 1H), 7.46-7.35 (m, 5H), 5.21 (s, 2H), 3.96-3.85 (m, 1H), 3.11 (t, J=5.28 Hz, 1H), 2.88 (dd, J=5.72, 2.32 Hz, 1H) ppm. ESI-MS m/z calc. 288.091, found 289.0 (M+1)⁺; Retention time: 1.75 minutes.

### Step 4:

A solution of benzyl (5-fluoro-2-(oxiran-2-yl)pyridin-4-yl)carbamate (2.2 g, 7.6316 mmol) in MeOH (35 mL) at 0° C. was saturated with NH₃ by purging with NH₃ gas for 10 min. The reaction mixture was sealed and stirred at ambient temperature for 24 h. The mixture was concentrated in vacuo. Purification by flash chromatography (SiO₂, 10% MeOH containing 2% NH₄OH in DCM) gave benzyl (2-(2-amino-1-hydroxyethyl)-5-fluoropyridin-4-yl)carbamate (1.6 g, 69%). ¹H NMR (400 MHz, DMSO-d₆) δ 8.38-8.33 (m, 1H), 8.14-7.98 (m, 1H), 7.47-7.33 (m, 5H), 5.49 (br s, 1H), 5.21 (s, 2H), 4.48 (dd, J=7.6, 3.8 Hz, 1H), 2.86-2.84 (m, 1H), 2.62-2.59 (m, 1H) ppm; NH and NH₂ amines not observed.

### Step 5:

2 M Na₂CO₃ (9.2 mL, 18.4 mmol) and chloroacetyl chloride (0.5 mL, 6.286 mmol) were successively added to a solution of benzyl (2-(2-amino-1-hydroxyethyl)-5-fluoropyridin-4-yl)carbamate (2.3 g, 4.89 mmol) in DCM (25 mL) at 0 to 5° C. and the reaction mixture was stirred at ambient temperature for 1 h. The mixture was diluted with water (100 mL) and extracted with DCM (500 mL). The organic extracts were dried (MgSO₄), filtered and concentrated in vacuo. Purification by flash chromatography (SiO₂, 30 to 50% EtOAc in heptane) gave benzyl (2-(2-(2-chloroacetamido)-1-hydroxyethyl)-5-fluoropyridin-4-yl)carbamate (1.3 g, 70%). ¹H NMR (400 MHz, DMSO-d₆) δ 10.12 (s, 1H), 8.38 (s, 1H), 8.20 (t, J=5.44 Hz, 1H), 8.13 (d, J=6 Hz, 1H), 7.46-7.35 (m, 5H), 5.75 (d, J=4.44 Hz, 1H), 5.22 (s, 2H), 4.62-4.60 (m, 1H), 4.05 (s, 2H), 3.55-3.52 (m, 1H), 3.19-3.16 (m, 1H) ppm.

### Step 6:

KO^{t}Bu (580 mg, 5.16 mmol) was added to a solution of benzyl (2-(2-(2-chloroacetamido)-1-hydroxyethyl)-5-fluoropyridin-4-yl)carbamate (1.3 g, 3.4 mmol) in a mixture of DCM (15 mL) and IPA (15 mL) at 0 to 5° C. The reaction mixture was heated at 50° C. for 16 h. The mixture was diluted with water (100 mL) and extracted with EtOAc (500 mL). The organic extracts were dried (MgSO₄), filtered and concentrated in vacuo. Purification by flash chromatography (SiO₂, 50 to 80% EtOAc in heptane) gave benzyl (5-fluoro-2-(5-oxomorpholin-2-yl)pyridin-4-yl)carbamate (850 mg, 72%) as white solid. ¹H NMR (400 MHz, DMSO-d₆) δ 10.22 (s, 1H), 8.43 (s, 1H), 8.16-8.14 (m, 2H), 7.46-7.35 (m, 5H), 5.22 (s, 2H), 4.82-4.79 (m, 1H), 4.23-4.19 (m, 2H), 3.56-3.53 (m, 1H), 3.32-3.28 (m, 1H) ppm.

### Step 7:

BH₃·THF (32 mL, 1 M solution in THF, 32 mmol) was added dropwise to a stirred solution of benzyl (5-fluoro-2-(5-oxomorpholin-2-yl)pyridin-4-yl)carbamate (1.8 g, 5.21 mmol) in THF (20 mL) at 0° C. under an argon atmosphere. The reaction mixture was heated at 40° C. for 30 min. The mixture was quenched by addition of MeOH and concentrated in vacuo. The residue was dissolved in HCOOH (20 mL) at 0° C. and NaBH₄ (610 mg, 16.124 mmol) was added. The reaction mixture was heated at ambient temperature for 1 h. The mixture was diluted with a saturated NaHCO₃ solution and extracted with EtOAc (500 mL). The organic extracts were dried (MgSO₄) and concentrated in vacuo. K₂CO₃ (2.2 g, 15.91 mmol) and Boc₂0 (3.42 g, 3.6 mL, 15.67 mmol) were successively added to the residue redissolved in a mixture of EtOAc (20 mL) and H₂O (10 mL). The reaction mixture was stirred at ambient temperature for 16 h. The mixture was diluted saturated NaHCO₃ solution and extracted with EtOAc (500 mL). The organic extracts were dried (MgSO₄) and concentrated in vacuo. Purification by flash chromatography (SiO₂, 50 to 80% EtOAc in heptane) gave tert-butyl 2-(4-(((benzyloxy)carbonyl)amino)-5-fluoropyridin-2-yl)morpholine-4-carboxylate (750 mg, 33%) as colourless oil. ¹H NMR (400 MHz, DMSO-d₆) δ 10.19 (s, 1H), 8.42 (d, J=2.52 Hz, 1H), 8.12 (d, J=6.64 Hz, 1H), 7.46-7.33 (m, 5H), 5.21 (s, 2H), 4.40-4.37 (m, 1H), 4.19-4.17 (m, 1H), 4.02-3.99 (m, 1H), 3.81 (br d, J=12.64 Hz, 1H), 3.63-3.57 (m, 1H), 2.95 (s, 1H), 2.74 (br s, 1H), 1.42 (s, 9H) ppm.

### Step 8:

Pd/C (50 mg, 10% w/w, 0.0412 mmol) was added to a degassed solution of tert-butyl 2-(4-(((benzyloxy)carbonyl)amino)-5-fluoropyridin-2-yl)morpholine-4-carboxylate (300 mg, 0.695 mmol) in a mixture of EtOAc (7 mL) and EtOH (7 mL). The reaction mixture was stirred under a hydrogen atmosphere for 4 h, filtered through a pad of Celite and concentrated in vacuo. Purification by flash chromatography (SiO₂, 20 to 30% EtOAc in hexanes) gave tert-butyl 2-(4-amino-5-fluoropyridin-2-yl)morpholine-4-carboxylate (150 mg, 71%) as an off-white solid. ¹H NMR (400 MHz, DMSO-d₆) δ 7.99 (d, J=3.36 Hz, 1H), 6.80 (d, J=7.76 Hz, 1H), 6.19 (s, 2H), 4.21-4.17 (m, 1H), 4.11 (br d, J=11.04 Hz, 1H), 3.92 (br d, J=11.48 Hz, 1H), 3.77 (br d, J=12.8 Hz, 1H), 3.56-3.50 (m, 1H), 2.88 (br s, 1H), 2.67 (br s, 1H), 1.39 (s, 9H) ppm. ESI-MS m/z calc. 297.1489, found 298.0 (M+1)⁺; Retention time: 1.64 minutes.

### Intermediate EH

### 1-(4-amino-5-fluoro-2-pyridyl)-2-(dimethylamino)ethanol

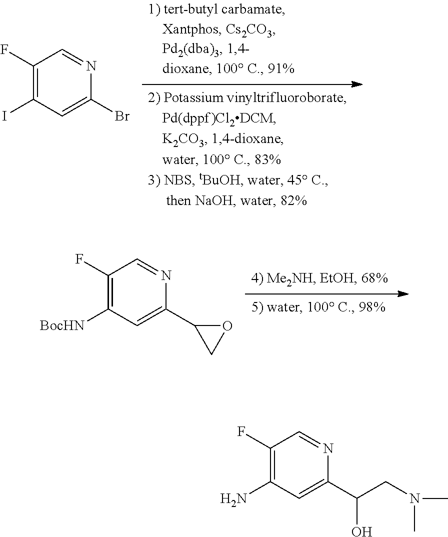 [SMILES: Fc1cnc(Br)cc1I ; CC(C)(C)OC(=O)Nc1cc(C2CO2)ncc1F ; CN(C)CC(O)c1cc(N)c(F)cn1]

### Steps 1, 2 and 3:

Steps 1 to 3 were carried out in a similar fashion to that described in Intermediate 62 using 2-bromo-5-fluoro-4-iodopyridine as the starting material to yield tert-butyl N-[5-fluoro-2-(oxiran-2-yl)-4-pyridyl]carbamate (874 mg, 82% in Step 3). ¹H NMR (400 MHz, Chloroform-d) δ 8.32 (d, J=2.4 Hz, 1H), 8.09 (d, J=6.4 Hz, 1H), 6.94 (s, 1H), 3.96 (dd, J=4.1, 2.5 Hz, 1H), 3.15 (dd, J=5.8, 4.1 Hz, 1H), 3.00 (dd, J=5.8, 2.5 Hz, 1H), 1.56 (s, 9H) ppm. ESI-MS m/z calc. 254.106, found 253.4 (M−1)⁻; Retention time: 1.59 minutes.

### Step 4:

To a solution of tert-butyl N-[5-fluoro-2-(oxiran-2-yl)-4-pyridyl]carbamate (75 mg, 0.295 mmol) in EtOH (0.5 mL) was added dimethylamine (750 μL of 40% w/w, 5.922 mmol). The reaction mixture was stirred at ambient temperature overnight. The mixture was concentrated in vacuo. Purification by flash chromatography (SiO₂, 0 to 25% EtOAc in EtOH containing 0.5% NH₄0H) gave tert-butyl N-[2-[2-(dimethylamino)-1-hydroxy-ethyl]-5-fluoro-4-pyridyl]carbamate (60.4 mg, 68%) as a yellow oil. ¹H NMR (500 MHz, Chloroform-d) δ 8.30 (d, J=6.6 Hz, 1H), 8.27 (d, J=2.3 Hz, 1H), 6.90 (s, 1H), 4.77 (dd, J=10.0, 3.7 Hz, 1H), 2.65 (dd, J=12.4, 3.7 Hz, 1H), 2.58 (dd, J=12.4, 10.0 Hz, 1H), 2.40 (s, 6H), 1.54 (s, 9H) ppm. ESI-MS m/z calc. 299.16452, found 299.9 (M+1)⁺; 298.0 (M−1)⁻; Retention time: 0.86 minutes.

### Step 5:

tert-Butyl N-[2-[2-(dimethylamino)-1-hydroxy-ethyl]-5-fluoro-4-pyridyl]carbamate (48.5 mg, 0.162 mmol) was dissolved in water (2 mL) and heated at 100° C. for 30 min. The reaction mixture was diluted with MeOH (5 mL) and concentrated in vacuo. The residue was dissolved with MeOH (5 mL), dried (MgSO₄) and concentration in vacuo to give 1-(4-amino-5-fluoro-2-pyridyl)-2-(dimethylamino)ethanol (31.5 mg, 98%) as a white solid. ESI-MS m/z calc. 199.112, found 199.8 (M+1)⁺; Retention time: 0.34 minutes.

The following intermediate was made using a method similar to that described in Intermediate EH, except that morpholine was used in Step 4:

| | | | |
|---|---|---|---|
| Inter- | | | NMR |
| mediate | | | (shifts |
| No. | Compound Name | LC/MS | in ppm) |

| | | |
|---|---|---|
| EI | 2-(1-((tert- | ESI-MS m/z calc. |
| | butyldimethylsilyl)oxy)- | 355.20914, found |
| | 2-morpholinoethyl)-5- | 357.0 (M + 1)⁺; |
| | fluoropyridin-4-amine | Retention time: |
| | | 1.07 minutes |

### Intermediate EJ

### tert-butyl (S)-3-(((tert-butyldimethylsilyl)oxy)methyl)-5-oxopiperazine-1-carboxylate

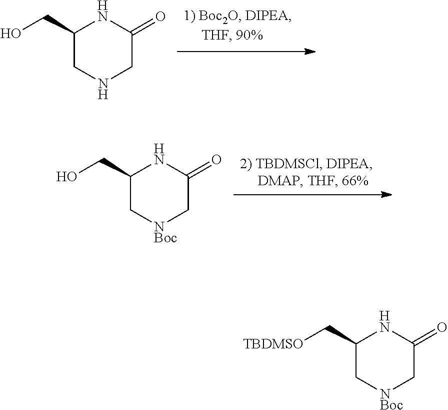 [SMILES: O=C1CNC[C@@H](CO)N1 ; CC(C)(C)OC(=O)N1CC(=O)N[C@H](CO)C1 ; CC(C)(C)OC(=O)N1CC(=O)N[C@H](CO[Si](C)(C)C(C)(C)C)C1]

### Step 1:

DIPEA (1.4 mL, 8.038 mmol) and Boc₂0 (1.0 mL, 4.353 mmol) were successively added to a stirred suspension of (S)-6-(hydroxymethyl)piperazin-2-one (500 mg, 3.842 mmol) in THF (10 mL) under N₂ at ambient temperature. The reaction mixture was stirred overnight and partitioned between EtOAc and a saturated NH₄Cl solution. The aqueous phase was separated and extracted with EtOAc. The combined organic extracts were washed with brine, dried (Na₂SO₄), filtered and concentrated in vacuo. Purification by flash chromatography (40 g SiO₂, 0 to 100% 3:1 EtOAc:EtOH in heptane) gave tert-butyl (S)-3-(hydroxymethyl)-5-oxopiperazine-1-carboxylate (798 mg, 90%) as a colourless oil which solidified on standing. ¹H NMR (500 MHz, DMSO-d₆) δ 7.94 (s, 1H), 4.88 (t, J=5.1 Hz, 1H), 3.82 (s, 2H), 3.66-3.34 (m, 3H), 3.30-3.12 (m, 2H), 1.42 (s, 9H) ppm. ESI-MS m/z calc. 230.127, found 231.0 (M+1)⁺; 229.0 (M−1)⁻; Retention time: 0.45 minutes.

### Step 2:

tert-Butylchlorodimethylsilane (630 mg, 4.180 mmol), DIPEA (1.2 mL, 6.889 mmol) and DMAP (42 mg, 0.344 mmol) were successively added to a solution of tert-butyl (S)-3-(hydroxymethyl)-5-oxopiperazine-1-carboxylate (798 mg, 3.466 mmol) in THF (8 mL) at ambient temperature under a N₂ atmosphere. The reaction mixture was stirred overnight then partitioned between EtOAc and water. The aqueous phase was separated and extracted with EtOAc. The combined organic extracts were washed with brine, dried (Na₂SO₄), filtered and concentrated in vacuo. Purification by flash chromatography (40 g SiO₂, 0 to 100% EtOAc in heptane) gave tert-butyl (S)-3-(((tert-butyldimethylsilyl)oxy)methyl)-5-oxopiperazine-1-carboxylate (793 mg, 66%) as a colourless oil which solidified on standing. ¹H NMR (500 MHz, DMSO-d₆) δ 7.93 (s, 1H), 3.82 (d, J=17.9 Hz, 1H), 3.69 (s, 1H), 3.59 (dd, J=13.3, 4.6 Hz, 1H), 3.50-3.45 (m, 1H), 3.43-3.33 (m, 2H), 1.36 (s, 9H), 0.82 (s, 9H), −0.00 (s, 6H) ppm. ESI-MS m/z calc. 344.213, found 345.0 (M+1)⁺; 343.0 (M−1)⁻; Retention time: 0.98 minutes.

The following intermediate was made using a method similar to that described in Intermediate EJ except that, in Step 1, (R)-6-(hydroxymethyl)piperazin-2-one was used as starting materials in place of (S)-6-(hydroxymethyl)piperazin-2-one:

| | | | |
|---|---|---|---|
| Inter- | | | NMR |
| mediate | | | (shifts |
| No. | Compound Name | LC/MS | in ppm) |

| | | |
|---|---|---|
| EK | tert-butyl (R)-3-(((tert- | ESI-MS m/z calc. |
| | butyldimethylsilyl)oxy)methyl)- | 344.213, found |
| | 5-oxopiperazine-1-carboxylate | 345.0 (M + 1)⁺; |
| | | 343.0(M − 1)⁻; |
| | | Retention time: |
| | | 1.0 minutes |

The following intermediates were made using a method similar to that described in Intermediate EJ except that, in Step 1, different starting materials were used as the starting material in place of (S)-6-(hydroxymethyl)piperazin-2-one and the reaction was carried out in 2-MeTHF in place of THF. Step 2 was omitted:

| Intermediate | | | |
|---|---|---|---|
| No. | Compound Name | LC/MS | NMR (shifts in ppm) |
| EL | tert-butyl methyl(2- | ESI-MS m/z calc. | ¹H NMR (500 MHz, |
| | oxopyrrolidin-3-yl)carbamate | 214.131, found | DMSO-d₆) δ 7.81 (d, J = |
| | | 215.0 (M − Boc)⁻; | 18.8 Hz, 1H), 4.62 (d, J = |
| | | Retention time: | 10.2 Hz, 0.5H), 4.32 (s, |
| | | 0.48 minutes | 0.5H), 3.23-3.09 (m, |
| | | | 2H), 2.68 (d, J = 12.7 Hz, |
| | | | 3H), 2.27-2.08 (m, 1H), |
| | | | 2.08-1.89 (m, 1H), 1.58- |
| | | | 1.27 (m, 9H) ppm. |
| EM | tert-butyl methyl(5- | ESI-MS m/z calc. | ¹H NMR (500 MHz, |
| | oxopyrrolidin-3-yl)carbamate | 214.132, found | DMSO-d₆) δ 7.64 (s, 1H), |
| | | 215.0 (M + 1)+; | 4.73 (s, 1H), 3.45 (ddd, |
| | | Retention time: | J = 10.2, 8.2, 0.9 Hz, 1H), |
| | | 0.47 minutes. | 3.13 (dd, J = 10.2, 5.0 Hz, |
| | | | 1H), 2.72 (s, 3H), 2.38 |
| | | | (dd, J = 17.1, 9.4 Hz, |
| | | | 1H), 2.20 (dd, J = 17.1, |
| | | | 6.1 Hz, 1H), 1.40 (s, 9H) |
| | | | ppm. |

### Intermediate EN

(S)-4,6-dimethylpiperazin-2-one

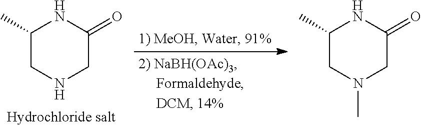

### Step 1:

(S)-6-methylpiperazin-2-one (hydrochloride salt) (459 mg, 3.048 mmol) was dissolved in a mixture of MeOH (3 mL) and water (2 mL) and loaded onto an SCX cartridge (5 g). The cartridge was washed with MeOH (25 mL) and the compound was released by washing the cartridge with 2 M methanolic ammonia (20 mL). The ammonia wash was concentrated in vacuo to give (S)-6-methylpiperazin-2-one (0.331 g, 91%) as an off-white solid. ¹H NMR (400 MHz, DMSO-d₆) δ 7.57 (s, 1H), 3.06 (dd, J=25.8, 17.3 Hz, 2H), 2.85 (dd, J=12.8, 4.1 Hz, 1H), 2.27 (dd, J=12.9, 8.1 Hz, 1H), 0.98 (d, J=6.4 Hz, 3H) ppm; NH amine and CH not observed.

### Step 2:

A N-methylation step was carried out according to General Method K, except that (S)-6-methylpiperazin-2-one was used as starting material and the reaction was carried out in DCM as the solvent. Purification by flash chromatography (12 g SiO₂, 0 to 10% MeOH in DCM) gave (S)-4,6-dimethylpiperazin-2-one (53.5 mg, 14%) as a pale yellow oil. ¹H NMR (400 MHz, Chloroform-d) δ 5.96 (s, 1H), 3.73-3.64 (m, 1H), 3.26 (dd, J=16.5, 1.4 Hz, 1H), 2.86-2.77 (m, 2H), 2.32 (s, 3H), 2.09 (dd, J=11.7, 8.7 Hz, 1H), 1.17 (d, J=6.4 Hz, 3H) ppm.

The following intermediates were made using a method similar to that described in Intermediate EN except that, Step 1 was omitted and, in Step 2, different starting materials were used in place of (S)-6-methylpiperazin-2-one:

| Intermediate | | | |
|---|---|---|---|
| No. | Compound Name | LC/MS | NMR (shifts in ppm) |
| EO | (R)-4,5-dimethylpiperazin-2- | | ¹H NMR (300 MHz, |
| | one | | Chloroform-d) δ 6.06 (br |
| | | | s, 1H), 3.39 (d, J = 16.8 |
| | | | Hz, 1H), 3.26 (dt, J = |
| | | | 11.5, 3.6 Hz, 1H), 3.15- |
| | | | 3.06 (m, 1H), 2.97 (d, J = |
| | | | 16.8 Hz, 1H), 2.59-2.48 |
| | | | (m, 1H), 2.29 (s, 3H), |
| | | | 1.13 (d, J = 6.4 Hz, 3H) |
| | | | ppm. |
| EP | (R)-4,6-dimethylpiperazin-2- | ESI-MS m/z calc. | ¹H NMR (500 MHz, |
| | one | 128.09496, found | DMSO-d₆) δ 7.74 (s, 1H), |
| | | 129.0 (M + 1)⁺; | 3.46 (dddd, J = 12.4, 7.8, |
| | | Retention time: | 5.3, 3.0 Hz, 1H), 2.95 |
| | | 0.2 minutes | (dd, J = 16.2, 1.4 Hz, |
| | | | 1H), 2.79-2.59 (m, 2H), |
| | | | 2.19 (s, 3H), 1.98 (dd, J = |
| | | | 11.5, 8.0 Hz, 1H), 1.06 |
| | | | (d, J = 6.3 Hz, 3H) ppm. |
| EQ | (R)-3,4-dimethylpiperazin-2- | | ¹H NMR (400 MHz, |
| | one | | Chloroform-d) δ 6.44 (br |
| | | | s, 1H), 3.45 (td, J = 10.8, |
| | | | 4.4 Hz, 1H), 3.27-3.19 |
| | | | (m, 1H), 2.93-2.80 (m, |
| | | | 2H), 2.56-2.50 (m, 1H), |
| | | | 2.37 (s, 3H), 1.38 (d, J = |
| | | | 6.9 Hz, 3H) ppm. |

### Intermediate ER

### tert-butyl (5)-2-methyl-5-oxopiperazine-1-carboxylate

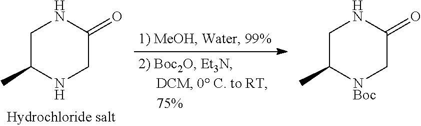

### Step 1:

(S)-5-Methylpiperazin-2-one hydrochloride (250 mg, 1.660 mmol) was dissolved in a mixture of MeOH (3 mL) and water (2 mL) and loaded onto an SCX cartridge (5 g). The cartridge was washed with MeOH (25 mL) and the compound was released by washing the cartridge with 2 M methanolic ammonia (20 mL). The ammonia wash was concentrated in vacuo to give (S)-5-methylpiperazin-2-one (198 mg, 99%) as a thick yellow oil. ¹H NMR (400 MHz, DMSO-d₆) δ 7.93 (s, 1H), 7.40 (br s, 1H), 3.44 (d, J=16.9 Hz, 1H), 3.37 (d, J=16.9 Hz, 1H), 3.23-3.15 (m, 2H), 3.03-2.97 (m, 1H), 1.13 (d, J=6.2 Hz, 3H) ppm.

### Step 2:

Boc₂O (170 mg, 0.179 mL, 0.779 mmol) and Et₃N (181.50 mg, 0.25 mL, 1.794 mmol) were successively added to an ice-cold solution of (S)-5-methylpiperazin-2-one (100 mg, 0.832 mmol) in DCM (1.6 mL). The reaction mixture was stirred for 1 h in the ice bath and then at ambient temperature for a further 21 h. The mixture was diluted with DCM (7.5 mL), washed with water (2×4 mL), 1 M HCl (6 mL), water (5 mL), saturated NaHCO₃ (5 mL), dried (Na₂SO₄), filtered and concentrated in vacuo to give tert-butyl (S)-2-methyl-5-oxopiperazine-1-carboxylate (140 mg, 75%) as a white solid. ¹H NMR (400 MHz, Methanol-d₄) δ 4.38-4.32 (m, 1H), 4.15 (d, J=18.3 Hz, 1H), 3.70 (d, J=18.5 Hz, 1H), 3.49 (dd, J=12.7, 4.5 Hz, 1H), 3.10 (dd, J=12.7, 1.9 Hz, 1H), 1.46 (s, 9H), 1.20 (d, J=6.6 Hz, 3H) ppm; NH amide not observed.

The following intermediate was made using a method similar to that described in Intermediate ER except that, in Step 1, (R)-5-methylpiperazin-2-one hydrochloride salt was used as starting materials in place of (5)-5-methylpiperazin-2-one hydrochloride:

| | | | |
|---|---|---|---|
| Intermediate | | | |
| No. | Compound Name | LC/MS | NMR (shifts in ppm) |

| | | |
|---|---|---|
| ES | tert-butyl (R)- | ¹H NMR (300 MHz, |
| | 2-methyl-5- | Methanol-d₄) δ 4.42- |
| | oxopiperazine- | 4.31 (m, 1H), 4.16 (d, J = |
| | 1-carboxylate | 18.4 Hz, 1H), 3.70 (d, J = |
| | | 18.4 Hz, 1H), 3.50 (dd, J = |
| | | 12.8, 4.4 Hz, 1H), 3.10 |
| | | (dd, J = 12.7, 2.1 Hz, |
| | | 1H), 1.46 (s, 9H), 1.21 (d, |
| | | J = 6.7 Hz, 3H) ppm; NH |
| | | amine not observed. |

The following intermediates were made using a method similar to that described in Intermediate ER except that, Step 1 was omitted and, in Step 2, different starting materials were used in place of (5)-5-methylpiperazin-2-one:

| | | | |
|---|---|---|---|
| Intermediate | | | |
| No. | Compound Name | LC/MS | NMR (shifts in ppm) |

| | | |
|---|---|---|
| ET | tert-butyl (R)- | ¹H NMR (400 MHz, |
| | 2-methyl-3- | Chloroform-d) δ 6.15 (s, |
| | oxopiperazine- | 1H), 4.58 (s, 1H), 4.12 (s, |
| | 1-carboxylate | 1H), 3.45 (td, J = 11.6, |
| | | 4.2 Hz, 1H), 3.24 (qt, J = |
| | | 6.0, 2.0 Hz, 1H), 3.18- |
| | | 3.11 (m, 1H), 1.47 (s, |
| | | 9H), 1.42 (d, J = 7.1 Hz, |
| | | 3H) ppm. |
| EU | tert-butyl (S)- | ¹H NMR (400 MHz, |
| | 2-methyl-3- | Chloroform-d) δ 6.13 (s, |
| | oxopiperazine- | 1H), 4.59 (s, 1H), 4.12 (s, |
| | 1-carboxylate | 1H), 3.45 (td, J = 11.6, |
| | | 4.1 Hz, 1H), 3.24 (qt, J = |
| | | 5.9, 2.0 Hz, 1H), 3.15 (t, |
| | | J = 11.8 Hz, 1H), 1.48- |
| | | 1.45 (m, 9H), 1.42 (d, J = |
| | | 7.1 Hz, 3H) ppm. |
| EV | tert-butyl (R)- | ¹H NMR (400 MHz, |
| | 3-methyl-5- | Methanol-d₄) δ 4.03 (d, |
| | oxopiperazine- | J = 18.3 Hz, 1H), 3.91 (d, |
| | 1-carboxylate | J = 18.3 Hz, 1H), 3.74 (dd, |
| | | J = 13.3, 3.7 Hz, 1H), |
| | | 3.60-3.52 (m, 1H), 3.22- |
| | | 3.08 (m, 1H), 1.46 (s, |
| | | 9H), 1.16 (d, J = 6.4 Hz, |
| | | 3H) ppm; NH amide not |
| | | observed. |
| EW | tert-butyl (S)- | ¹H NMR (400 MHz, |
| | 3-methyl-5- | Methanol-d₄) 8 4.03 (d, |
| | oxopiperazine- | J = 18.3 Hz, 1H), 3.91 (d, |
| | 1-carboxylate | J = 18.3 Hz, 1H), 3.74 (dd, |
| | | J = 13.5, 3.2 Hz, 1H), |
| | | 3.60-3.52 (m, 1H), 3.15 |
| | | (br s, 1H), 1.46 (s, 9H), |
| | | 1.16 (d, J = 6.4 Hz, 3H) |
| | | ppm; NH amide not |
| | | observed. |

### Intermediate EX

(S)-4,5-dimethylpiperazin-2-one

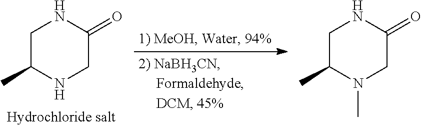

### Step 1:

(S)-5-Methylpiperazin-2-one hydrochloride (250 mg, 1.66 mmol) was dissolved in a mixture of MeOH (2 mL) and water (2 mL) and loaded onto an SCX cartridge (5 g). The cartridge was washed with MeOH (25 mL) and the compound was released by washing the cartridge with 2 M methanolic ammonia (20 mL). The ammonia wash was concentrated in vacuo to give (S)-5-methylpiperazin-2-one (187 mg, 94%) as an off-white solid. ¹H NMR (400 MHz, DMSO-d₆) δ 7.55 (s, 1H), 3.17 (dd, J=19.9, 17.2 Hz, 2H), 3.10-3.06 (m, 1H), 2.86-2.76 (m, 2H), 2.57 (br s, 1H), 1.00 (d, J=6.0 Hz, 3H) ppm.

### Step 2:

NaBH₃CN (88 mg, 1.4 mmol) was added to a solution of (S)-5-methylpiperazin-2-one (125 mg, 1.073 mmol) and formaldehyde (0.110 mL, 37% w/w solution in water, 1.481 mmol) in MeOH (3 mL). The reaction mixture was stirred at ambient temperature for 18 before being concentrated in vacuo. The residue was partitioned between 15% IPA in DCM (15 mL) and a saturated aqueous NaHCO₃ solution (10 mL). The aqueous layer was separated and extracted with 15% IPA in DCM (2×15 mL). The combined organic extracts were dried (Na₂SO₄), filtered and concentrated in vacuo to give a yellow oil. Purification by flash chromatography (12 g, SiO₂, 0 to 15% MeOH in DCM) gave (S)-4,5-dimethylpiperazin-2-one (65 mg, 45%) as a pale yellow oil. ¹H NMR (400 MHz, Chloroform-d) δ 6.17 (s, 1H), 3.40-3.35 (d, J=16.8 Hz, 1H), 3.25 (dt, J=11.5, 3.6 Hz, 1H), 3.13-3.07 (m, 1H), 2.95 (d, J=16.8 Hz, 1H), 2.58-2.48 (m, 1H), 2.28 (s, 3H), 1.12 (d, J=6.4 Hz, 3H) ppm.

The following intermediate was made using a method similar to that described in Intermediate EX except that, Step 1 was omitted. In Step 2, (S)-3-methylpiperazin-2-one was used as the starting material in place of (S)-5-methylpiperazin-2-one and the reaction was carried out in MeOH in place of DCM:

| | | | |
|---|---|---|---|
| Inter- | | | |
| mediate | | | |
| No. | Compound Name | LC/MS | NMR (shifts in ppm) |

| | | |
|---|---|---|
| EY | (S)-3,4-dimethylpiperazin- | ¹H NMR (400 MHz, |
| | 2-one | Chloroform-d) δ 6.14 (s, |
| | | 1H), 3.47 (td, J = 10.8, |
| | | 4.4 Hz, 1H), 3.27-3.21 |
| | | (m, 1H), 2.96-2.83 (m, |
| | | 2H), 2.58-2.51 (m, 1H), |
| | | 2.38 (s, 3H), 1.39 (d, J = |
| | | 6.9 Hz, 3H) ppm. |

### Intermediate EZ

### tert-butyl methyl(2-(methylamino)-2-oxoethyl)carbamate

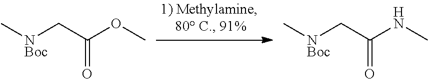

### Step 1:

A stirred solution of methyl N-(tert-butoxycarbonyl)-N-methylglycinate (460 mg, 2.263 mmol) in methylamine (3 mL, 33% w/v solution in ethanol, 31.877 mmol) was heated at 80° C. under microwave irradiations for 1 h. The reaction mixture was concentrated in vacuo, then azeotroped with acetonitrile (5 mL) to give tert-butyl methyl(2-(methylamino)-2-oxoethyl)carbamate (440 mg, 91%) as a white solid. ¹H NMR (300 MHz, Chloroform-d) δ 6.12 (br s, 1H), 3.85 (s, 2H), 2.94-2.89 (m, 3H), 2.83 (d, J=4.8 Hz, 3H), 1.43 (d, J=17.0 Hz, 9H) ppm. ESI-MS m/z calc. 202.1317, found 103.1 (M−99)⁻, Retention time: 0.64 minutes.

### Intermediate FA

### tert-butyl (2-amino-2-oxoethyl)(methyl)carbamate

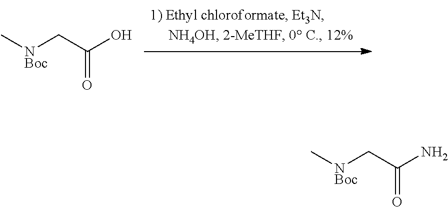

### Step 1:

Ethyl chloroformate (627 mg, 0.55 mL, 5.778 mmol) was added to an ice-cold solution of N-(tert-butoxycarbonyl)-N-methylglycine (1 g, 5.285 mmol) and Et₃N (653.4 mg, 0.9 mL, 6.457 mmol) in 2-MeTHF (20 mL) and the solution was stirred for 20 min. NH₄OH (0.65 mL, 28% w/v solution in water, 10.687 mmol) was added and the reaction mixture was stirred at ambient temperature overnight. The mixture was washed with 10% citric acid solution (20 mL) and a saturated sodium bicarbonate solution (20 mL), dried (Na₂SO₄) and concentrated in vacuo to give a white solid (200 mg). Purification by flash chromatography (12 g SiO₂, 0 to 10% MeOH in EtOAc) gave tert-butyl (2-amino-2-oxoethyl)(methyl)carbamate (132 mg, 12%) as a white solid. ¹H NMR (400 MHz, Chloroform-d) δ 6.14 (s, 2H), 3.82 (s, 2H), 2.91 (s, 3H), 1.43 (s, 9H) ppm. ESI-MS m/z calc. 188.1161, found 89.0 (M−99)⁻; Retention time: 0.58 minutes.

### Intermediate FB

### tert-butyl (1S,4S)-6-oxo-2,5-diazabicyclo[2.2.1]heptane-2-carboxylate

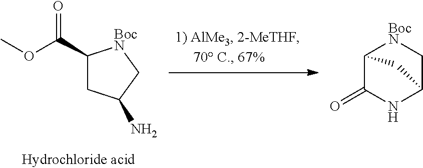 [SMILES: COC(=O)[C@@H]1C[C@H](N)CN1C(=O)OC(C)(C)C ; CC(C)(C)OC(=O)N1C[C@@H]2C[C@H]1C(=O)N2]

### Step 1:

Trimethylaluminium (1.8 mL, 2 M solution in heptane, 3.6 mmol) was added to a stirred suspension of 1-(tert-butyl) 2-methyl (2S,4S)-4-aminopyrrolidine-1,2-dicarboxylate hydrochloride (504 mg, 1.795 mmol) in 2-MeTHF (15 mL). The reaction mixture was stirred at ambient temperature for 20 min to give a clear solution. An additional amount of trimethylaluminium (0.9 mL, 2 M solution in heptane, 1.8 mmol) was added and the reaction mixture was stirred at 70° C. for 30 min. The reaction was quenched by addition of a 10% citric acid solution (15 mL) and extracted with EtOAc (15 mL). The organic layer was separated, dried (Na₂SO₄) and concentrated in vacuo to give tert-butyl (1S,4S)-6-oxo-2,5-diazabicyclo[2.2.1]heptane-2-carboxylate (279 mg, 67%) as a white solid. H NMR (400 MHz, Chloroform-d) δ 5.53 (br s, 1H), 4.38 (br s, 1H), 4.10 (s, 1H), 3.46 (dd, J=9.8, 1.6 Hz, 1H), 3.26 (s, 1H), 2.02 (d, J=9.2 Hz, 1H), 1.84 (d, J=8.2 Hz, 1H), 1.45 (s, 9H) ppm. ESI-MS m/z calc. 212.1161, found 157.0 (M−55)⁻; Retention time: 0.66 minutes.

### Intermediate FC

### tert-butyl (1R,4R)-6-oxo-2,5-diazabicyclo[2.2.1]heptane-2-carboxylate

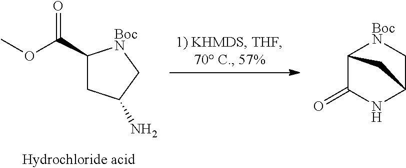 [SMILES: COC(=O)[C@@H]1C[C@@H](N)CN1C(=O)OC(C)(C)C ; CC(C)(C)OC(=O)N1C[C@H]2C[C@@H]1C(=O)N2]

### Step 1:

KHMDS (0.6 mL, 1 M solution in THF, 0.6 mmol) was added to a suspension of 1-(tert-butyl) 2-methyl (2S,4R)-4-aminopyrrolidine-1,2-dicarboxylate hydrochloride (50 mg, 0.178 mmol) in THF (3 mL). The solution was stirred at 70° C. for 30 min then at ambient temperature overnight. The mixture was concentrated in vacuo. The residue was dissolved in EtOAc (15 mL), washed with 1 M HCl (2×5 mL) and brine (5 mL), dried (Na₂SO₄), filtered and concentrated in vacuo to give tert-butyl (1R,4R)-6-oxo-2,5-diazabicyclo[2.2.1]heptane-2-carboxylate (24 mg, 57%) as a light brown solid. ¹H NMR (400 MHz, Chloroform-d) δ 6.30-5.82 (m, 1H), 4.62-4.31 (m, 1H), 4.10 (s, 1H), 3.45 (dd, J=9.6, 1.4 Hz, 1H), 3.29-3.00 (m, 1H), 2.03-1.99 (m, 1H), 1.88-1.75 (m, 1H), 1.46 (s, 9H) ppm. ESI-MS m/z calc. 212.1161, found 157.1 (M−55)⁺; Retention time: 0.68 minutes.

### Intermediate FD

### N-(4-aminopyridin-2-yl)-N-methylacetamide

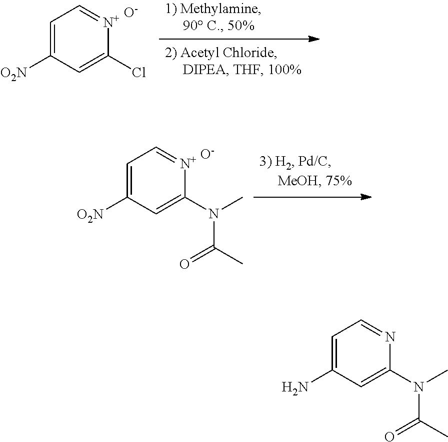

### Step 1:

2-Chloro-4-nitropyridine 1-oxide (300 mg, 1.719 mmol) was combined with methylamine (3 mL, 2 M solution in MeOH, 6 mmol) and the mixture stirred at 90° C. for 20 min under microwave irradiations. The mixture was concentrated in vacuo. Purification by reverse phase HPLC (C18 X-bridge column, MeCN in H₂O with 0.1% ammonium hydroxide) gave 2-(methylamino)-4-nitropyridine 1-oxide (144 mg, 50%) as a bright orange solid. ¹H NMR (500 MHz, DMSO-d₆) δ 8.34 (dd, J=6.5, 0.9 Hz, 1H), 7.70 (d, J=5.6 Hz, 1H), 7.40 (d, J=6.7 Hz, 2H), 2.95 (d, J=5.1 Hz, 3H) ppm. ESI-MS m/z calc. 169.04874, found 170.0 (M+1)⁺; Retention time: 0.35 minutes.

### Step 2:

DIPEA (300 μL, 1.722 mmol) and acetyl chloride (100 μL, 1.406 mmol) were successively added to a stirred solution of 2-(Methylamino)-4-nitropyridine 1-oxide (144 mg, 0.851 mmol) in THF (3 mL) under N₂ at ambient temperature. The reaction mixture was stirred for 10 min at ambient temperature. The mixture was partitioned between EtOAc and water. The aqueous phase was separated and extracted with EOAc (×4). The combined organic extracts were dried (Na₂SO₄), filtered and concentrated in vacuo to give 2-(N-methylacetamido)-4-nitropyridine 1-oxide (179 mg, 100%) as a yellow oil. ¹H NMR (500 MHz, DMSO-d₆) δ 9.06-8.48 (m, 2H), 8.28 (dd, J=7.2, 3.3 Hz, 1H), 3.14 (s, 3H), 1.93 (s, 3H) ppm. ESI-MS m/z calc. 211.05931, found 212.0 (M+1)⁺; Retention time: 0.28 minutes.

### Step 3:

A solution of 2-(N-methylacetamido)-4-nitropyridine 1-oxide (179 mg, 0.848 mmol) in MeOH (5 mL) was de-gassed (×2 vacuum−N₂ cycles). Pd/C (100 mg of 10% w/w, 0.094 mmol) was added with stirring and the mixture was de-gassed (×2 vacuum−N₂ cycles). The N₂ atmosphere was replaced with H₂ (×3 vacuum−H₂ cycles) and the mixture was stirred for 90 min. An additional amount of Pd/C (100 mg of 10% w/w, 0.094 mmol) was added after de-gassing with N₂. The nitrogen atmosphere was replaced with a H₂ atmosphere (×w3 vacuum-H₂ cycles) and the mixture was stirred for 90 min. The mixture was placed under N₂ and was filtered. The filtrate was concentrated in vacuo to give N-(4-aminopyridin-2-yl)-N-methylacetamide (132 mg, 75%) as a pale brown oil. ¹H NMR (500 MHz, DMSO-d₆) δ 7.88 (d, J=5.7 Hz, 1H), 6.41 (d, J=5.5 Hz, 2H), 6.20 (s, 2H), 3.12 (s, 3H), 1.91 (s, 3H) ppm. ESI-MS m/z calc. 165.09021, found 166.0 (M+1)⁺; Retention time: 0.28 minutes.

### Intermediate FE

(4-amino-5-fluoropyridin-2-yl)methanol

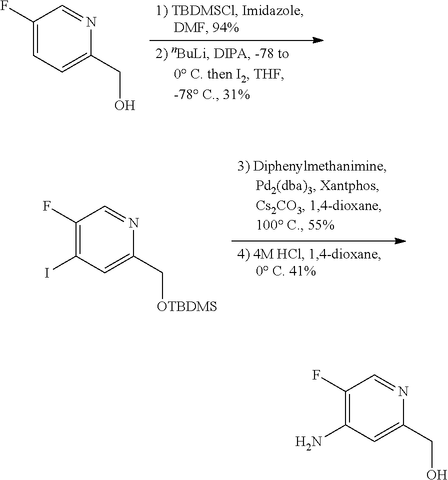 [SMILES: OCc1ccc(F)cn1 ; CC(C)(C)[Si](C)(C)OCc1cc(I)c(F)cn1 ; Nc1cc(CO)ncc1F]

### Step 1:

Imidazole (25.8 g, 378.98 mmol) and TBDMSCl (30.1 g, 199.71 mmol) were successively added to a solution of (5-fluoropyridin-2-yl)methanol (24.15 g, 189.98 mmol) in DMF (190 mL) and the solution was stirred at ambient temperature for 20 min. The mixture was quenched by addition of a saturated NH₄Cl solution (100 mL) and 0.1 M HCl (700 mL). The mixture was extracted with EtOAc (3×500 mL). The combined organic extracts were washed with water (3×1 L) and brine (300 mL), dried (Na₂SO₄), filtered, and concentrated in vacuo. Purification by flash chromatography (330 g SiO₂, 0 to 1% EtOAc in hexanes) gave 2-(((tert-butyldimethylsilyl)oxy)methyl)-5-fluoropyridine (43.16 g, 94%) as a colourless oil. ¹H NMR (250 MHz, DMSO-d₆) δ 8.49 (m, 1H), 7.75 (m, 1H), 7.49 (m, 1H), 4.75-4.73 (m, 2H), 0.91 (s, 9H), 0.09 (s, 6H) ppm. ESI-MS m/z calc. 241.1298, found 242.7 (M+1)⁺; Retention time: 6.31 minutes.

### Step 2:

ⁿBuLi (50 mL, 1.73 M solution in hexanes, 86.5 mmol) was slowly added to a stirred solution of DIPA (9.747 g, 13.5 mL, 96.324 mmol) in THF (150 mL) at −78° C. and the reaction mixture was stirred at 0° C. for 30 min. A solution of 2-(((tert-butyldimethylsilyl)oxy)methyl)-5-fluoropyridine (16 g, 66.286 mmol) in THF (150 mL) was slowly added at −78° C. and the mixture was stirred for 1 h at −78° C. A solution of I₂ (16.8 g, 66.192 mmol) in THF (150 mL) was added dropwise at −78° C. and the reaction mixture was stirred for 1 h at −78° C. The mixture was quenched by addition of an aqueous NH₄Cl solution (200 mL). The mixture was warmed to ambient temperature and extracted with EtOAc (2×250 mL). The combined organic extracts were washed with brine (100 mL), dried (Na₂SO₄), filtered and concentrated in vacuo. Purification by flash chromatography (SiO₂, 3 to 5% EtOAc in n-hexane) gave 2-(((tert-butyldimethylsilyl)oxy)methyl)-5-fluoro-4-iodopyridine (8.2 g, 31%) as an off-white solid. ¹H NMR (400 MHz, DMSO-d₆) δ 8.41 (s, 1H), 7.86 (d, J=5.04 Hz, 1H), 4.72 (s, 2H), 0.90 (s, 9H), 0.09 (s, 6H) ppm. ESI-MS m/z calc. 367.027, found 368.1 (M+1)⁺; Retention time: 2.68 minutes.

### Step 3:

Diphenylmethanimine (110 mg, 0.607 mmol) was added to a stirred suspension of 2-(((tert-butyldimethylsilyl)oxy)methyl)-5-fluoro-4-iodopyridine (200 mg, 0.545 mmol) and Cs₂CO₃ (532 mg, 1.633 mmol) in 1,4-dioxane (6 mL). The reaction mixture was degassed with N₂ before addition of xantphos (64 mg, 0.111 mmol) and Pd₂(dba)₃ (50 mg, 0.055 mmol). The reaction mixture was heated at 100° C. for 5 h. The reaction was cooled to ambient temperature. Water and sodium chloride were added and the mixture was extracted with EtOAc. The organic phase was dried (Na₂SO₄), filtered, and concentrated in vacuo. Purification by flash chromatography (SiO₂, 2 to 5% EtOAc in ⁿhexane) gave N-(2-(((tert-butyldimethylsilyl)oxy)methyl)-5-fluoropyridin-4-yl)-1,1-diphenylmethanimine (130 mg, 55%). ¹H NMR (400 MHz, DMSO-d₆) δ 8.26 (d, J=2.1 Hz, 1H), 7.70 (s, 2H), 7.51 (s, 3H), 7.40-7.35 (m, 3H), 7.16 (s, 2H), 6.83 (d, J=6.6 Hz, 1H), 4.56 (s, 2H), 0.86 (s, 9H), −0.06 (s, 6H) ppm. ESI-MS m/z calc. 420.2033, found 421.2 (M+1)⁺; Retention time: 2.41 minutes.

### Step 4:

4 M HCl (0.3 mL, solution in 1,4-dioxane, 1.2 mmol) was added to a stirred solution of a N-(2-(((tert-butyldimethylsilyl)oxy)methyl)-5-fluoropyridin-4-yl)-1,1-diphenylmethanimine (130 mg, 0.309 mmol) in 1,4-dioxane (2 mL) at 0° C. The reaction mixture was stirred at ambient temperature overnight. The mixture was quenched by addition of water. The organic phase was separated and washed with an aqueous saturated NaHCO₃ solution (0.6 mL). The aqueous phase was extracted with EtOAc (15 mL), dried (Na₂SO₄) and concentrated in vacuo. Purification by flash chromatography (SiO₂, 15 to 18% EtOAc in ⁿhexane) gave (4-amino-5-fluoropyridin-2-yl)methanol (18 mg, 41%). ¹H NMR (400 MHz, DMSO-d₆) δ 7.95 (d, J=3.2 Hz, 1H), 6.80 (d, J=7.7 Hz, 1H), 6.18 (s, 2H), 5.23 (t, J=5.9 Hz, 1H), 4.33 (d, J=5.64 Hz, 2H) ppm.

### Intermediate FF

### 3-methoxy-[1,2,4]triazolo[4,3-a]pyridin-7-amine

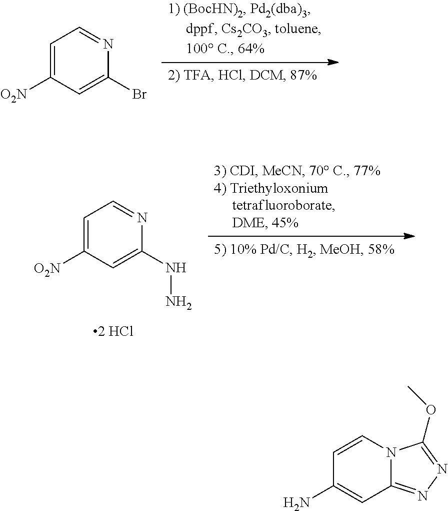

### Step 1:

A mixture of 2-bromo-4-nitro-pyridine (15.1 g, 74.386 mmol), di-tert-butyl hydrazine-1,2-dicarboxylate (17.4 g, 74.911 mmol), Pd₂(dba)₃ (5.5 g, 6.006 mmol), dppf (5 g, 9.019 mmol), and Cs₂CO₃ (24.4 g, 74.888 mmol) in toluene (200 mL) was stirred at 100° C. for 16 h. The mixture was cooled to ambient temperature, filtered, and the cake was rinsed with EtOAc (200 mL). The filtrate was concentrated in vacuo. Purification by flash chromatography (SiO₂, 0 to 40% EtOAc in hexanes) gave di-tert-butyl 1-(4-nitropyridin-2-yl)hydrazine-1,2-dicarboxylate (16.9 g, 64%) as brown sticky gum. ESI-MS m/z calc. 354.1539, found 355.7 (M+1)⁺; Retention time: 3.42 minutes.

### Step 2:

TFA (213.12 g, 144 mL, 1.869 mol) was added to a solution of di-tert-butyl 1-(4-nitropyridin-2-yl)hydrazine-1,2-dicarboxylate (34 g, 95.948 mmol) in DCM (500 mL) and the reaction mixture was stirred at ambient temperature for 70 min. 2 M HCl (950 mL, solution in diethyl ether, 1.9 mol) was added to the mixture followed by 250 mL of hexane. The formed precipitate was collected by filtration and dried in vacuo to give 2-hydrazineyl-4-nitropyridine (2 Hydrochloride salt) (21.1 g, 87%) as a yellow solid. ESI-MS m/z calc. 154.0491, found 155.2 (M+1)⁺; Retention time: 0.92 minutes.

### Step 3:

CDI (1.5 g, 9.251 mmol) was added to a stirred solution of 2-hydrazineyl-4-nitropyridine (1 g, 6.488 mmol) in MeCN (20 mL) and the reaction mixture was stirred at 70° C. for 16 h. The mixture was concentrated in vacuo and the residue was partitioned between water and EtOAc. The organic phase was separated and concentrated in vacuo. Purification by flash chromatography (SiO₂, 0 to 90% EtOAc in hexanes) gave 7-nitro-[1,2,4]triazolo[4,3-a]pyridin-3(2H)-one (900 mg, 77%) as a white solid. ¹H NMR (400 MHz, DMSO-d₆) δ 12.5 (s, 1H), 7.76 (d, J=7.3 Hz, 1H), 7.61 (s, 1H), 6.65 (d, J=7.1 Hz, 1H) ppm.

### Step 4:

Triethyloxonium tetrafluoroborate (3.7 g, 25.016 mmol) was added to a solution of 7-nitro-[1,2,4]triazolo[4,3-a]pyridin-3(2H)-one (3.01 g, 16.711 mmol) in 1,2-dimethoxyethane (165 mL) and the reaction mixture was stirred at ambient temperature overnight. The mixture was quenched by addition of an aqueous saturated NaHCO₃ solution (70 mL) and extracted with a 3:1 mixture of DCM and ⁱPrOH (9×70 mL). The combined organic extracts were dried (Na₂SO₄), filtered and concentrated in vacuo. Purification by flash chromatography (80 g SiO₂, 0 to 15% DCM in MeOH) gave 3-methoxy-7-nitro-[1,2,4]triazolo[4,3-a]pyridine (1.46 g, 45%) as red solid. ¹H NMR (250 MHz, DMSO-d₆) δ 8.89 (s, 1H), 8.26 (d, J=7.4 Hz, 1H), 7.46 (d, J=8.0 Hz, 1H), 3.96 (s, 3H) ppm. ESI-MS m/z calc. 194.044, found 195.7 (M+1)⁺; Retention time: 0.97 minutes.

### Step 5:

To a solution of 3-methoxy-7-nitro-[1,2,4]triazolo[4,3-a]pyridine (1.46 g, 7.52 mmol) in MeOH (115 mL) was added 10% Pd/C (405 mg, 3.806 mmol). The reaction mixture was degassed and placed under a hydrogen atmosphere (balloon). The reaction mixture was stirred at ambient temperature for 4 h. The mixture was filtered through a pad of Celite, washing with MeOH (50 mL). The filtrate was concentrated in vacuo. Purification by flash chromatography (SiO₂, 0 to 25% DCM in MeOH) gave 3-methoxy-[1,2,4]triazolo[4,3-a]pyridin-7-amine (749.4 mg, 58%) as pale orange solid. ¹H NMR (500 MHz, DMSO-d₆) δ 7.76 (dd, J=7.3, 0.6 Hz, 1H), 6.83 (s, 2H), 6.37 (dd, J=7.3, 1.9 Hz, 1H), 6.08 (dd, J=1.9, 0.7 Hz, 1H), 3.45 (s, 3H) ppm. ESI-MS m/z calc. 164.0698, found 165.0 (M+1)⁺; Retention time: 0.49 minutes.

### Example 34

### E-VIPR Assay Detecting and Measuring Na_{V} Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Na_{V} 1.8 with full channel activity were seeded into microtiter 384-well plates, pre-coated with matrigel, at a density of 25,000 cells per well. 2.5-5% KIR2.1 Bacmam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R210-01). Cells were expanded in vented cap cell culture flasks, with 90-95% humidity and 5% CO₂.

### Reagents and Stock Solutions:

100 mg/mL Pluronic F-127 (Sigma #P2443), in dry DMSO

Compound Plates: Corning 384-well Polypropylene Round Bottom #3656

Cell Plates: 384-well tissue culture treated plates (Greiner #781091-2B)

2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.

5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6(1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.

5 mM CC2-DMPE, a commercially available membrane-bound coumarin phospholipid FRET donor (ThermoFisher Scientific catalog number K1017, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl]ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.

Voltage Assay Background Suppression Compound (VABSC-1) is prepared in H₂O (89-363 mM, range used to maintain solubility)

Human Serum (HS, Millipore #S1P1-01KL, or Sigma SLBR5469V and SLBR5470V as a 50%/50% mixture, for 25% assay final concentration)
Bath 1 Buffer:
Sodium Chloride 160 mM (9.35 g/L), Potassium Chloride, 4.5 mM (0.335 g/L), Glucose 10 mM (1.8 g/L), Magnesium Chloride (Anhydrous) 1 mM (0.095 g/L), Calcium Chloride 2 mM (0.222 g/L), HEPES 10 mM (2.38 g/L) in water.
Na/TMA C₁ Bath 1 Buffer:
Sodium Chloride 96 mM (5.61 g/L), Potassium Chloride 4.5 mM (0.335 g/L), Tetramethylammonium (TMA)-Cl 64 mM (7.01 g/L), Glucose 10 mM (1.8 g/L), Magnesium Chloride (Anhydrous) 1 mM (0.095 g/L), Calcium Chloride 2 mM (0.222 g/L) HEPES 10 mM (2.38 g/L) in water.
Hexyl Dye Solution (2× concentration):
Bath 1 Buffer containing 0.5% P-cyclodextrin (made fresh prior to each use, Sigma #C4767), 8 μM CC2-DMPE and 2 μM DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath 1/p-Cyclodextrin.

Compound Loading Buffer (2× concentration): Na/TMA C₁ Bath1 Buffer containing HS (omitted in experiments run in the absence of human serum (HS)) 50%, VABSC-1 1 mM, BSA 0.2 mg/ml (in Bath-1), KCl 9 mM, DMSO 0.625%.

### Assay Protocol (7 Key Steps):

1) To reach the final concentration in each well, 375 nL of each compound was pre-spotted (in neat DMSO) into polypropylene compound plates at 240× desired final concentration from an intermediate stock concentration of 0.075 mM, in an 11 point dose response, 3-fold dilution, resulting in a top dose of 300 nM final concentration in the cell plate. Vehicle control (neat DMSO), and positive control (an established Na_{V}1.8 inhibitor, 25 μM final in assay in DMSO) were added manually to the outermost columns of each plate respectively. The compound plate was backfilled with 45 μL per well of Compound Loading Buffer resulting in a 240 fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.

2) Hexyl Dye Solution was prepared.

3) Cell plates were prepared. On the day of the assay, the media was aspirated, and the cells were washed three times with 80 μL of Bath-1 buffer, maintaining 25 μL residual volume in each well.

4) 25 μL per well of Hexyl Dye Solution was dispensed into the cell plates. The cells were incubated for 20 minutes at room temperature or ambient conditions in darkness.

5) 45 μL per well of Compound Loading Buffer was dispensed into compound plates.

6) The cell plates were washed three times with 80 μL per well of Bath-1 Buffer, leaving L of residual volume. Then 25 μL per well from compound plate was transferred to each cell plate. The mixture was incubated for 30 minutes at room temperature/ambient conditions.

7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

### Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 nm} )}{( {intensity}_{580 nm} )}$$

The data were further reduced (i.e. normalized) by calculating the initial (Rᵢ) and final (R_{f}) ratios. These were the average ratio values during part or all of the pre-stimulation period and during sample points during the stimulation period. The fluorescence ratio (R_{f}/Rᵢ) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the ratio response of the test compound (i.e. the maximum amplitude of the ratio response or number of action potential peaks, at the beginning of the pulse train in the presence of the test compound). Using this analysis protocol, dose response curves were plotted and IC₅₀ values were generated for various compounds of the present invention as reported below.

Compounds having a measured IC₅₀ value less than 0.01 μM in the E-VIPR Assay described above include: 1, 3, 8, 9*, 12, 14, 18, 19, 22, 23, 24, 26, 28, 30, 32, 36, 38, 42, 44, 46, 48, 50, 51, 54, 55, 60, 62, 64, 74, 78, 80, 82, 84, 85, 90, 92, 94, 96, 97, 98, 100, 101, 103, 104, 105, 107, 108, 110, 111, 112, 113, 119, 120, 121, 123, 125, 128, 129, 130, 131, 132, 134, 135, 137, 144, 145*, 148*, 151, 152, 153, 156, 161, 162, 168, 170, 173, 174, 176, 180, 183, 184, 188, 190, 196, 197, 199, 200, 201, 202, 203, 204, 205, 208, 209, 211, 216, 219, 220, 221, 222, 223, 226, 227, 230, 231, 232, 233, 234, 239, 240, 246, 248, 249*, 250, 251, 252, 253, 254, 255, 256, 257, 262, 265, 267, 311, 315, 316, 327, 328, 329, 330, 332, 333, 336, 340, 341, 344, 345, 346, 349, 352, 353, 354, 355, 358, 359, 360, 361, 362, 364, 365, 366, 368, 369, 370, 377, 378, 379, 384, 385, 386, 411, 412, 413, 418, 419, 423, 424, 425, 428, 429, 432, 439, 440, 449, 451, 454, 456, 458, 465, 469, 470, 474, 480, 485, 486, 487, 495, 503, 511, 512, 516, 517, 518, 519, 522, 523, 528, 530, 532, 534, 536, 537, 538, 540, 558, 564, 566, 567, 575, 576, 577, 579, 580, 584, 599, 601, 602, 621, 622, 625, 639, 641, 642, 643, 646, 647, 648, 651, 652, 654, 675, 676, 682, 684, and 733.

Compounds having a measured IC₅₀ value less than 0.1 μM and greater than or equal to 0.01 μM in the E-VIPR Assay described above include: 2, 5, 13*, 15, 16, 17, 20, 21, 34, 57, 65*, 68, 72, 76, 140*, 141*, 147*, 155, 158, 165, 167, 171, 179, 181, 182, 187, 189, 191, 192, 193, 194, 195, 198, 206*, 207*, 210, 213, 214, 215, 217, 218, 228, 229, 235, 236, 237, 238, 241, 242, 243*, 244, 245, 258, 259, 260, 261, 264, 268, 310, 312, 313, 314, 320, 324, 325, 331, 334, 337, 338, 339, 342, 343, 347, 348, 350, 351, 356, 357, 363, 367, 371, 372, 374, 376, 380, 381, 383, 387, 388, 389, 390, 395, 398, 399, 401, 402, 403, 405, 408, 410, 417, 422, 430, 431, 436, 437, 438, 442, 443, 446, 447, 453, 460, 464, 466, 467, 468, 471, 472, 473, 475, 476, 477, 478, 479, 481, 482, 483, 484, 488, 489, 490, 491, 492, 493, 494, 496, 497, 498, 499, 500, 501, 502, 504, 505, 506, 507, 508, 509, 510, 513, 514, 515, 520, 521, 524, 525, 526, 535, 539, 541, 544, 545, 548, 555, 557, 559, 560, 565, 568, 570, 571, 572, 574, 578, 585, 586, 587, 588, 589, 590, 591, 592, 593, 595, 596, 597, 598, 600, 605, 607, 608, 609, 610, 611, 614, 615, 617, 618, 619, 620, 623, 627, 628, 629, 630, 631, 632, 633, 634, 635, 636, 637, 638, 640, 644, 645, 650, 653, 655, 656, 658, 659, 660, 661, 662, 663, 664, 665, 666, 667, 668, 669, 670, 672, 677, 680, 681, 685*, 687*, 694*, 696*, 699*, 700*, 701, 702, 704, 706, 710, 711, and 732.

Compounds having a measured IC₅₀ value less than 1 μM and greater than or equal to 0.1 μM in the E-VIPR Assay described above include: 10, 39, 40, 45, 53, 56, 79, 70*, 81, 88*, 89,106,109, 114, 116, 117, 122, 124, 126, 127, 133, 149*, 150, 160, 163, 175, 212*, 224, 225, 247, 263, 270, 280, 281, 317, 373, 375, 382, 391, 392, 393, 394, 397, 400, 404, 409, 414, 415, 421, 426, 427, 433, 434, 435, 444, 450, 455, 462, 527, 529, 547, 549, 550, 556, 569, 573, 581, 582, 583, 594, 604, 606, 612, 613, 616, 624, 626, 649, 671, 673, 674, 678, 689*, 691*, 708*, 714*, 715*, 717*, 718, 720, 721*, 731, and 734.

Compounds having a measured IC₅₀ value greater than or equal to 1 μM in the E-VIPR Assay described above include: 4, 6*, 7*, 11, 25, 27*, 29, 31, 33, 35, 37, 41, 43, 47, 49*, 52, 58*, 59, 61, 63*, 66*,67*,69*,71,73,75,77,83*,86*,87*,91,93*,95*,99, 102, 115, 118, 136*, 138*, 139*, 142*, 143*, 146*, 154, 157, 159, 164, 169, 172, 177, 178, 185*, 186, 266, 269, 271, 272, 273, 274, 275, 276, 277, 278, 279, 282, 283, 284, 285, 286, 287, 288, 289, 290, 291, 292, 293, 294, 296, 297, 298, 299, 300, 301, 302, 303, 304, 305, 306, 307, 308, 309, 318, 319, 321, 322, 323, 335, 396, 406, 407, 420, 445, 448, 452, 457*, 459, 461, 463, 531*, 533*, 542, 543, 546, 551, 552, 553, 554, 561, 562, 563, 603, 657, 679, 683*,686*,688*,690*,692*,693*,695*,697*,698*,703*,705*,707*,709*,712*,713*,716*,719*, 722*, 723*, 724*, 725*, 726*, 727*, 728*, 729*, 730*, and 735.

An IC₅₀ value was not determined in the E-VIPR Assay described for Compounds 166, 295, 326, 416, and 441.

Compound numbers followed by “*” indicate that the assay was performed in the presence of human serum, as described above.

Many modifications and variations of the embodiments described herein may be made without departing from the scope, as is apparent to those skilled in the art. The specific embodiments described herein are offered by way of example only.

What is claimed is:

1. A compound of formula (I) 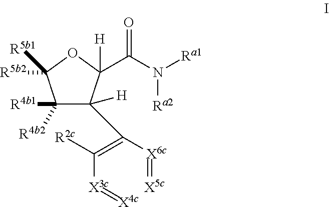
or a pharmaceutically acceptable salt thereof, wherein:
R^{a1} is —(C^{a′})₂)ₚ—R^{a″}, 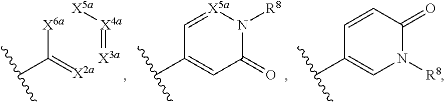
5-membered heteroaryl, 3-7 membered heterocycloalkyl, 9-10 membered aryl, or 9-10 membered heteroaryl, wherein said 5-membered heteroaryl, 3-7 membered heterocycloalkyl, 9-10 membered aryl, or 9-10 membered heteroaryl is optionally substituted by one or more R^{a3};
R^{a2} is H;
or R^{a1} and R^{a2} together with the nitrogen to which they are attached form a 3-10 membered heterocycloalkyl, wherein said 3-10 membered heterocycloalkyl is optionally substituted by one or more R^{a3};
each R^{a′} is independently H or methyl optionally substituted by OH, or two R^{a′} together with the atom or atoms to which they are attached form C₃-C₆ cycloalkyl, 3-7 membered heterocycloalkyl, or oxo;
R^{a″} is C₃-C₆ cycloalkyl, 3-7 membered heterocycloalkyl, 5-10 membered heteroaryl, phenyl, —NR⁹R¹⁰, —OR¹¹, or —CN, wherein said 5-10 membered heteroaryl, 3-7 membered heterocycloalkyl, or phenyl is optionally substituted by one or more R¹³;
each R^{a3} is independently halo, C₁-C₆ alkyl, C₁-C₆ haloalkyl, 3-7 membered heterocycloalkyl, —C(O)C₁-C₆ alkyl, —OR¹¹, —C(O)NR⁹R¹⁰, or —S(O)₂R⁷, wherein said C₁-C₆ alkyl, C₁-C₆ haloalkyl, 3-7 membered heterocycloalkyl or —C(O)C₁-C₆ alkyl is optionally substituted by one or more halo, —OR¹¹, —CN, or —NR⁹R¹⁰, or two R^{a3} attached to the same atom combine to form oxo, or two R^{a3} attached to adjacent atoms together with the atoms to which they are attached combine to form a fused 3-7 membered ring containing up to two heteroatoms selected from the group consisting of N, O, and S;
X^{2a} is N, N⁺—O—, or C—R^{2a};
X^{3a} is N, N⁺—O—, or C—R^{3a};
X^{4a} is N, N⁺—O—, or C—R^{4a};
X^{5a} is N, N⁺—O—, C—R^{5a}, or N⁺—(C₁-C₆ alkyl)Y⁻, wherein Y⁻ is a monovalent anion;
X^{6a} is N, N⁺—O—, or C—R^{6a};
R^{2a} is H, halo, C₁-C₆ alkyl, or C₁-C₆ haloalkyl;
R^{3a} is H, halo, C₁-C₆ alkyl, C₁-C₆ haloalkyl, C₁-C₆ alkoxy, 3-9 membered heterocycloalkyl, 5-membered heteroaryl, —CN, —OR¹¹, —COOH, —NR⁹C(O)C₁-C₆ alkyl, —S(O)₂R⁷, —S(O)(NR⁹)R⁷, —S(O)NR⁹R¹⁰, —S(O)R⁷, or —P(O)(C₁-C₆ alkyl)₂, wherein said C₁-C₆ alkyl, C₁-C₆ alkoxy, 3-9 membered heterocycloalkyl, 5-membered heteroaryl, or —NR⁹C(O)C₁-C₆ alkyl is optionally substituted by one or more R¹², C₃-C₆ cycloalkyl, —NR⁹R¹⁰, —OR¹¹, —CN, or 3-7 membered heterocycloalkyl optionally substituted by one or more R²;
R^{4a} is H, halo, C₁-C₆ alkyl, C₁-C₆ haloalkyl, C₂-C₆ alkynyl, C₁-C₆ alkoxy, 3-7 membered heterocycloalkyl, 5-6 membered heteroaryl, —CN, —C(O)NR⁹R¹⁰, —C(O)OH, —OR¹¹, —NR⁹R¹⁰, —NR⁹C(O)C₁-C₆ alkyl, —S—C₁-C₆ alkyl, —S(O)(NR⁹)R⁷, —S(O)NR⁹R¹⁰, or —P(O)(C₁-C₆ alkyl)₂, wherein said C₁-C₆ alkyl, C₁-C₆ alkoxy, 3-7 membered heterocycloalkyl, 5-6 membered heteroaryl, or C₂-C₆ alkynyl is optionally substituted by one or more halo, —OR¹¹, 3-7 membered heterocycloalkyl, —NR⁹R¹⁰, C₁-C₆ alkyl, or —S(O)₂R⁷;
R^{5a} is H, halo, C₁-C₆ alkyl, C₁-C₆ haloalkyl, or —S(O)₂R⁷;
R^{6a} is H, halo, C₁-C₆ alkyl, or C₁-C₆ haloalkyl;
or R^{3a} and R^{4a} together with the atoms to which they are attached form a ring of formula: 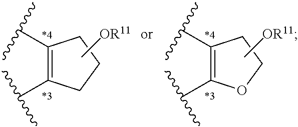
R⁷ is C₁-C₆ alkyl or 3-7 membered heterocycloalkyl, wherein said C₁-C₆ alkyl or 3-7 membered heterocycloalkyl is optionally substituted by one or more —OR¹⁰ or C₁-C₆ alkyl;
R⁸ is H or C₁-C₆ alkyl;
R⁹ and R¹⁰ are each independently H, C₁-C₆ alkyl, 3-7 membered heterocycloalkyl, C₃-C₆ cycloalkyl, —OH, —CN, or —S(O)₂R⁷, wherein said C₁-C₆ alkyl is optionally substituted by one or more —OR¹¹, or R⁹ and R¹⁰ together with the atom to which they are attached form a 37 membered heterocycloalkyl;
each R¹¹ is independently H, C₁-C₆ alkyl, C₁-C₆ haloalkyl, a 3-7 membered heterocycloalkyl optionally substituted with —OH, or a 3-7 membered cycloalkyl optionally substituted with —OH;
each R¹² is independently halo, C₁-C₆ alkyl, or —OR¹¹, or two R¹² together with the atom they are attached combine to form oxo;
each R¹³ is independently halo, C₁-C₆ alkyl, or —CONH₂, wherein said C₁-C₆ alkyl is optionally substituted by one or more —OR¹¹, or two R¹³ together with the atom they are attached combine to form oxo;
R^{4b1} and R^{4b2} are each independently H, C₁-C₆ alkyl, C₃-C₆ cycloalkyl, or C₁-C₆ haloalkyl;
R^{5b} and R^{5b2} are each independently H, C₁-C₆ alkyl, C₃-C₆ cycloalkyl, or C₁-C₆ haloalkyl;
X^{3c} is N or C—R^{3c};
X^{4c} is N or C—R^{4c};
X^{5c} is N or C—R^{5c};
X^{6c} is N or C—R^{6c};
R^{2c} is H, —OH, halo, C₁-C₆ alkyl, C₂-C₆ alkenyl, C₁-C₆ haloalkyl, C₁-C₆ alkoxy, C₁-C₆ haloalkoxy, -L¹-(C₁-C₆ alkylene)-OR⁵, -L¹-(C₁-C₆ alkenylene)-OR¹⁵, -L¹-(C₁-C₆ alkylene)-NR¹⁶R¹⁷, -L¹-(C₁-C₆ alkylene)-N═S(O)(C₁-C₃ alkyl)₂, or L¹-L2-R¹⁴;
R¹⁴ is C₃-C₆ cycloalkyl, 3-8 membered heterocycloalkyl, 5- or 6-membered heteroaryl, —C(O)O(C₁-C₆ alkyl), —COOH, or —C(O)NR¹⁶R¹⁷, wherein said C₃-C₆ cycloalkyl, 3-8 membered heterocycloalkyl or 5- or 6-membered heteroaryl is optionally substituted by one or more halo, —OH, C₁-C₆ alkyl, C₁-C₆ haloalkyl, C₁-C₆ alkoxy, or C₁-C₆ haloalkoxy;
R¹⁵ is H, C₁-C₆ alkyl, or C₁-C₆ haloalkyl:
R¹⁶ and R¹⁷ are each independently H, —OH, C₁-C₆ alkyl, or 3-7 membered heterocycloalkyl;
R^{3c} is H, halo, C₁-C₆ alkyl, C₁-C₆ haloalkyl, or -(C₁-C₆ alkylene)-(C₁-C₆ alkoxy);
R^{4c} is H, halo, C₁-C₆ alkyl, or C₁-C₆ haloalkyl;
R^{5c} is H, halo, C₁-C₆ alkyl, or C₁-C₆ haloalkyl; and
R^{6c} is H, halo, C₁-C₆ alkyl, C₁-C₆ haloalkyl, or C₁-C₆ alkoxy;
L¹ is a bond or O;
L² is a bond or C₁-C₆ alkylene; and
p is 1, 2, or 3;
provided that no more than two of X^{2a}, X^{3a}, X^{4a}, X^{5a}, and X^{6a} are N or N⁺—O;
provided that no more than one of X^{3c}, X^{4c}, X^{5c}, and X^{6c} is N; and
provided that R^{4a} is not CH(OH)—R^{4a′}, wherein when R^{4a′} is H or C₁-C₅ alkyl optionally substituted by one or more halo, —OR¹¹, 3-7 membered heterocycloalkyl, —NR⁹R¹⁰, C₁-C₆ alkyl, or —S(O)₂R⁷.

2. The compound of claim 1, wherein the compound has formula (I-A) 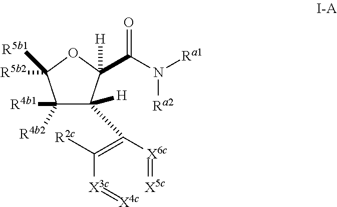
or a pharmaceutically acceptable salt thereof.

3. The compound of claim 1, wherein the compound has formula (I-A-1) 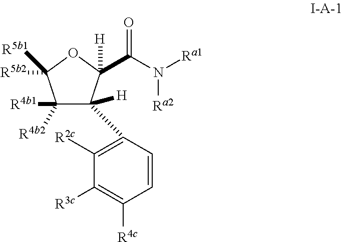
or a pharmaceutically acceptable salt thereof.

4. The compound of claim 1, wherein the compound has formula (I-B) 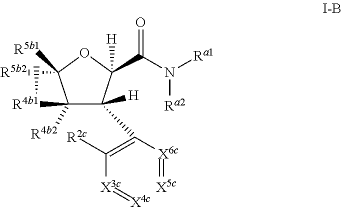
or a pharmaceutically acceptable salt thereof.

5. The compound of claim 1, wherein the compound has formula (I-B-1) 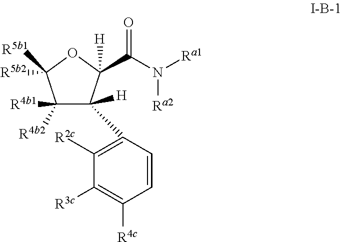
or a pharmaceutically acceptable salt thereof.

6. The compound of any one of claims 1-5, or the pharmaceutically acceptable salt thereof, wherein R^{a1} is 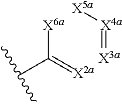
and R^{a2} is H.

7. The compound of any one of claims 1-5, or the pharmaceutically acceptable salt thereof, wherein R^{a1} is 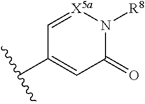
and R^{a2} is H.

8. The compound of any one of claims 1-5, or the pharmaceutically acceptable salt thereof, wherein R^{a1} is 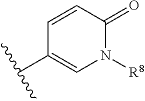
and R^{a2} is H.

9. The compound of any one of claims 1-5, or the pharmaceutically acceptable salt thereof, wherein R^{a1} is a 5-membered heteroaryl, a 9-10 membered aryl, or a 9-10 membered heteroaryl, wherein the 5-membered heteroaryl, 9-10 membered aryl, or 9-10 membered heteroaryl is optionally substituted by one or more R^{a3}; and R^{a2} is H.

10. The compound of claim 6, or the pharmaceutically acceptable salt thereof, wherein X^{2a} is C—R^{2a} and R^{2a} is H; X⁵a C—R^{5a} and R^{5a} is H; and X^{6a} is C—R^{6a} and R^{6a} is H.

11. The compound of any one of claims 1-6 or 10, or the pharmaceutically acceptable salt thereof, wherein X^{3a} is N or C-R^{3a}, wherein R^{3a} is —OR¹¹, —COOH, —S(O)₂R⁷, —S(O)(NR⁹)R⁷, —S(O)NR⁹R¹⁰, or —S(O)R⁷.

12. The compound of any one of claims 1-6, 10, or 11, or the pharmaceutically acceptable salt thereof, wherein X^{4a} is N.

13. The compound of any one of claims 1-5, 7, or 8, or the pharmaceutically acceptable salt thereof, wherein X^{5a} is C—R^{5a} and R^{5a} is H.

14. The compound of any one of claims 1-5 or 9, or the pharmaceutically acceptable salt thereof, wherein R^{a1} is a 5-membered heteroaryl or a 9-10 membered heteroaryl, wherein the 5-membered heteroaryl or 9-10 membered heteroaryl is optionally substituted by one or more R^{a3}; and R^{a2} is H.

15. The compound of any one of claims 1-6, 9-12, or 14, or the pharmaceutically acceptable salt thereof, wherein R⁷ is methyl; and R⁸ is H or methyl.

16. The compound of any one of claims 1-15, or the pharmaceutically acceptable salt thereof, wherein R^{2c} is CH₃ or OCH₃.

17. The compound of any one of claims 1-16, or a pharmaceutically acceptable salt thereof, wherein R^{3c} is halo, optionally F, or C₁-C₆ alkyl, optionally CH₃.

18. The compound of any one of claims 1-17, or a pharmaceutically acceptable salt thereof, wherein R^{4c} is halo, optionally F.

19. The compound of any one of claims 1-18, or a pharmaceutically acceptable salt thereof, wherein R^{5c} is H.

20. The compound of any one of claims 1-19, or a pharmaceutically acceptable salt thereof, wherein R^{6c} is H.

21. The compounds of any one of claims 1-20, or a pharmaceutically acceptable salt thereof, wherein one of R^{4b1} and R^{4b2} is H and one is methyl.

22. The compounds of any one of claims 1-20, or a pharmaceutically acceptable salt thereof, wherein one of R^{5b1} and R^{5b2} is methyl and one is trifluoromethyl.

23. A compound selected from Table A, or a pharmaceutically acceptable salt thereof.

24. The compound of any one of claims 1-23 in non-salt form.

25. A pharmaceutical composition comprising a therapeutically effective amount of the compound of any one of claims 1-23, or a pharmaceutically acceptable salt thereof, or the compound of claim 24 and one or more pharmaceutically acceptable carriers or vehicles.

26. A pharmaceutical composition comprising the compound of any one of claims 1-23, or a pharmaceutically acceptable salt thereof, or the compound of claim 24 and one or more pharmaceutically acceptable carriers or vehicles.

27. A method of inhibiting a voltage-gated sodium channel in a subject comprising administering to the subject the compound of any one of claims 1-23, or a pharmaceutically acceptable salt thereof, the compound of claim 24, or the pharmaceutical composition of claim 25 or 26.

28. The method of claim 27, wherein the voltage-gated sodium channel is Na_{V}1.8.

29. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of any one of claims 1-23, or a pharmaceutically acceptable salt thereof, the compound of claim 24, or the pharmaceutical composition of claim 25 or 26.

30. The method of claim 32, where the method comprises treating or lessening the severity in the subject of one or more of neuropathic pain, musculoskeletal pain preferably osteoarthritis pain, acute pain preferably acute post-operative pain, postsurgical pain, or visceral pain.

31. The method of claim 30, wherein the neuropathic pain comprises of one or more of post-herpetic neuralgia, small-fiber neuropathy, idiopathic small-fiber neuropathy, or diabetic neuropathy preferably diabetic peripheral neuropathy.

32. The method of claim 30, wherein the postsurgical pain comprises one or more of bunionectomy pain, abdominoplasty pain, or herniorrhaphy pain.

33. The method of any one of claims 27-32, wherein said subject is treated with one or more additional therapeutic agents administered concurrently with, prior to, or subsequent to treatment with the compound, pharmaceutically acceptable salt, or pharmaceutical composition.

34. Use of the compound of any one of claims 1-23, or a pharmaceutically acceptable salt thereof, the compound of claim 24, or the pharmaceutical composition of claim 25 or 26, as a medicament.
